(19) United States
(12) Patent Application Publication
(10) Pub. No.: US 2004/0067505 A1
(43) Pub. Date: Apr. 8, 2004

(54) Therapeutic polypeptides, nucleic acids encoding same, and methods of use

(76) Inventors: Enrique Alvarez, Clinton, CT (US); David Anderson, Branford, CT (US); Ferenc Boldog, North Haven, CT (US); Elina Catterton, Milford, CT (US); Shlomit R. Edinger, New Haven, CT (US); Elma Fernandes, Branford, CT (US); Valerie Gerlach, Branford, CT (US); Linda Gorman, Branford, CT (US); William Grosse, Branford, CT (US); Xiaojia (Sasha) Guo, Branford, CT (US); Weizhen Ji, Branford, CT (US); Ramesh Kekuda, Norwalk, CT (US); Li Li, Branford, CT (US); John R. MacDougall, Hamden, CT (US); Muralidhara Padigaru, Branford, CT (US); Meera Patturajan, Branford, CT (US); Jeffery D. Peterson, Brookfield, CT (US); Luca Rastelli, Guilford, CT (US); Richard Shimkets, Guilford, CT (US); Kimberly Spytek, Ellington, CT (US); David J. Stone, Guilford, CT (US); Corine A.M. Vernet, Branford, CT (US); Edward Voss, Wallingford, CT (US); Mei Zhong, Branford, CT (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/428,275

(22) Filed: May 1, 2003

Related U.S. Application Data
(63) Continuation-in-part of application No. 09/966,545, filed on Sep. 26, 2001.

(51) Int. Cl. C12Q 1/68; C07H 21/04; C07K 14/705; C12P 21/02; C12N 5/06; C07K 16/28

(52) U.S. Cl. 435/6; 435/69.1; 435/320.1; 435/325; 530/350; 530/388.22; 536/23.5

(57) ABSTRACT

The present invention provides novel isolated polynucleotides and small molecule target polypeptides encoded by the polynucleotides. Antibodies that immunospecifically bind to a novel small molecule target polypeptide or any derivative, variant, mutant or fragment of that polypeptide, polynucleotide or antibody are disclosed, as are methods in which the small molecule target polypeptide, polynucleotide and antibody are utilized in the detection and treatment of a broad range of pathological states. More specifically, the present invention discloses methods of using recombinantly expressed and/or endogenously expressed proteins in various screening procedures for the purpose of identifying therapeutic antibodies and therapeutic small molecules associated with diseases. The invention further discloses therapeutic, diagnostic and research methods for diagnosis, treatment, and prevention of disorders involving any one of these novel human nucleic acids and proteins.

### RELATED APPLICATIONS

This application is a continuation-in-part of U.S. Ser. No. 09/966,545, filed Sep. 26, 2001, which claims priority to U.S. Ser. No. 09/544,511, filed Apr. 6, 2000, which in turn claims priority to U.S. S No. 60/128,514, filed Apr. 9, 1999; and is a continuation-in-part of U.S. Ser. No. 09/569,269, filed May 11, 2000, which claims priority to U.S. S No. 60/134,315, filed May 14, 1999; and is a continuation-in-part of U.S. Ser. No. 09/619,252, filed Jul. 19, 2000, which claims priority to No. 60/144,722, filed Jul. 20, 1999; and is a continuation-in-part of U.S. Ser. No. 09/789,390, filed Feb. 23, 2001, which claims priority to U.S. S No. 60/185,548, filed Feb. 25, 2000; and is a continuation-in-part of U.S. Ser. No. 09/715,427, filed Nov. 16, 2000, which claims priority to U.S. S No. 60/233,798, filed Sep. 19, 2000; and this application claims the benefit of U.S. S No. 60/384,215, filed May 30, 2002; U.S. S No. 60/378,730 filed May 8, 2002; U.S. S No. 60/384,327, filed May 30, 2002; U.S. S No. 60/402,154, filed Aug. 6, 2002; U.S. S No. 60/406,129, filed Aug. 27, 2002; U.S. S No. 60/412,954, filed Sep. 23, 2002; U.S. S No. 60/384,352, filed May 30, 2002; U.S. S No. 60/402,205, filed Aug. 9, 2002; U.S. S No. 60/384,297, filed May 30, 2002; U.S. S No. 60/384,296, filed May 30, 2002; U.S. S No. 60/405,175, filed Aug. 22, 2002; U.S. S No. 60/402,204, filed Aug. 9, 2002; U.S. S No. 60/385,211, filed May 31, 2002; U.S. S No. 60/420,851, filed Oct. 24, 2002; U.S. S No. 60/384,044, filed May 29, 2002; U.S. S No. 60/383,075, filed May 24, 2002; U.S. S No. 60/393,333, filed Jul. 2, 2002; U.S. S No. 60/377,321, filed May 2, 2002; U.S. S No. 60/414,975, filed Sep. 30, 2002; U.S. S No. 60/416,661, filed Oct. 7, 2002, each of which is incorporated herein by reference in its entirety.

### FIELD OF THE INVENTION

The present invention relates to novel polypeptides, and the nucleic acids encoding them, having properties related to stimulation of biochemical or physiological responses in a cell, a tissue, an organ or an organism. More particularly, the novel polypeptides are gene products of novel genes, or are specified biologically active fragments or derivatives thereof. Methods of use encompass diagnostic and prognostic assay procedures as well as methods of treating diverse pathological conditions.

### BACKGROUND OF THE INVENTION

Eukaryotic cells are characterized by biochemical and physiological processes which under normal conditions are exquisitely balanced to achieve the preservation and propagation of the cells. When such cells are components of multicellular organisms such as vertebrates, or more particularly organisms such as mammals, the regulation of the biochemical and physiological processes involves intricate signaling pathways. Frequently, such signaling pathways involve extracellular signaling proteins, cellular receptors that bind the signaling proteins, and signal transducing components located within the cells.

Signaling proteins may be classified as endocrine effectors, paracrine effectors or autocrine effectors. Endocrine effectors are signaling molecules secreted by a given organ into the circulatory system, which are then transported to a distant target organ or tissue. The target cells include the receptors for the endocrine effector, and when the endocrine effector binds, a signaling cascade is induced. Paracrine effectors involve secreting cells and receptor cells in close proximity to each other, for example two different classes of cells in the same tissue or organ. One class of cells secretes the paracrine effector, which then reaches the second class of cells, for example by diffusion through the extracellular fluid. The second class of cells contains the receptors for the paracrine effector; binding of the effector results in induction of the signaling cascade that elicits the corresponding biochemical or physiological effect. Autocrine effectors are highly analogous to paracrine effectors, except that the same cell type that secretes the autocrine effector also contains the receptor. Thus the autocrine effector binds to receptors on the same cell, or on identical neighboring cells. The binding process then elicits the characteristic biochemical or physiological effect.

Signaling processes may elicit a variety of effects on cells and tissues including by way of nonlimiting example induction of cell or tissue proliferation, suppression of growth or proliferation, induction of differentiation or maturation of a cell or tissue, and suppression of differentiation or maturation of a cell or tissue.

Many pathological conditions involve dysregulation of expression of important effector proteins. In certain classes of pathologies the dysregulation is manifested as diminished or suppressed level of synthesis and secretion of protein effectors. In other classes of pathologies the dysregulation is manifested as increased or up-regulated level of synthesis and secretion of protein effectors. In a clinical setting a subject may be suspected of suffering from a condition brought on by altered or mis-regulated levels of a protein effector of interest. Therefore there is a need to assay for the level of the protein effector of interest in a biological sample from such a subject, and to compare the level with that characteristic of a nonpathological condition. There also is a need to provide the protein effector as a product of manufacture. Administration of the effector to a subject in need thereof is useful in treatment of the pathological condition. Accordingly, there is a need for a method of treatment of a pathological condition brought on by a diminished or suppressed levels of the protein effector of interest. In addition, there is a need for a method of treatment of a pathological condition brought on by a increased or up-regulated levels of the protein effector of interest.

Small molecule targets have been implicated in various disease states or pathologies. These targets may be proteins, and particularly enzymatic proteins, which are acted upon by small molecule drugs for the purpose of altering target function and achieving a desired result. Cellular, animal and clinical studies can be performed to elucidate the genetic contribution to the etiology and pathogenesis of conditions in which small molecule targets are implicated in a variety of physiologic, pharnacologic or native states. These studies utilize the core technologies at CuraGen Corporation to look at differential gene expression, protein-protein interactions, large-scale sequencing of expressed genes and the association of genetic variations such as, but not limited to, single nucleotide polymorphisms (SNPs) or splice variants in and between biological samples from experimental and control groups. The goal of such studies is to identify potential avenues for therapeutic intervention in order to prevent, treat the consequences or cure the conditions.

In order to treat diseases, pathologies and other abnormal states or conditions in which a mammalian organism has been diagnosed as being, or as being at risk for becoming, other than in a normal state or condition, it is important to identify new therapeutic agents. Such a procedure includes at least the steps of identifying a target component within an affected tissue or organ, and identifying a candidate therapeutic agent that modulates the functional attributes of the target. The target component may be any biological macromolecule implicated in the disease or pathology. Commonly the target is a polypeptide or protein with specific functional attributes. Other classes of macromolecule may be a nucleic acid, a polysaccharide, a lipid such as a complex lipid or a glycolipid; in addition a target may be a sub-cellular structure or extra-cellular structure that is comprised of more than one of these classes of macromolecule. Once such a target has been identified, it may be employed in a screening assay in order to identify favorable candidate therapeutic agents from among a large population of substances or compounds.

Antibodies are multichain proteins that bind specifically to a given antigen, and bind poorly, or not at all, to substances deemed not to be cognate antigens. Antibodies are comprised of two short chains termed light chains and two long chains termed heavy chains. These chains are constituted of immunoglobulin domains, of which generally there are two classes: one variable domain per chain, one constant domain in light chains, and three or more constant domains in heavy chains. The antigen-specific portion of the immunoglobulin molecules resides in the variable domains; the variable domains of one light chain and one heavy chain associate with each other to generate the antigen-binding moiety. Antibodies that bind immunospecifically to a cognate or target antigen bind with high affinities. Accordingly, they are useful in assaying specifically for the presence of the antigen in a sample. In addition, they have the potential of inactivating the activity of the antigen.

Therefore there is a need to assay for the level of a protein effector of interest in a biological sample from such a subject, and to compare this level with that characteristic of a nonpathological condition. In particular, there is a need for such an assay based on the use of an antibody that binds immunospecifically to the antigen. There further is a need to inhibit the activity of the protein effector in cases where a pathological condition arises from elevated or excessive levels of the effector based on the use of an antibody that binds immunospecifically to the effector. Thus, there is a need for the antibody as a product of manufacture. There further is a need for a method of treatment of a pathological condition brought on by an elevated or excessive level of the protein effector of interest based on administering the antibody to the subject.

### SUMMARY OF THE INVENTION

The invention is based in part upon the discovery of isolated polypeptides including amino acid sequences selected from mature forms of the amino acid sequences selected from the group consisting of SEQ ID NO:2n, wherein n is an integer between 1 and 174. The novel nucleic acids and polypeptides are referred to herein as NOV1a, NOV1b, NOV1c, NOV1d, NOV2a, NOV2b, NOV2c, NOV2d, NOV3a, NOV3b, etc. These nucleic acids and polypeptides, as well as derivatives, homologs, analogs and fragments thereof, will hereinafter be collectively designated as “NOVX” nucleic acid or polypeptide sequences.

The invention also is based in part upon variants of a mature form of the amino acid sequence selected from the group consisting of SEQ ID NO:2n, wherein n is an integer between 1 and 174, wherein any amino acid in the mature form is changed to a different amino acid, provided that no more than 15% of the amino acid residues in the sequence of the mature form are so changed. In another embodiment, the invention includes the amino acid sequences selected from the group consisting of SEQ ID NO:2n, wherein n is an integer between 1 and 174. In another embodiment, the invention also comprises variants of the amino acid sequence selected from the group consisting of SEQ ID NO:2n, wherein n is an integer between 1 and 174 wherein any amino acid specified in the chosen sequence is changed to a different amino acid, provided that no more than 15% of the amino acid residues in the sequence are so changed. The invention also involves fragments of any of the mature forms of the amino acid sequences selected from the group consisting of SEQ ID NO:2n, wherein n is an integer between 1 and 174, or any other amino acid sequence selected from this group. The invention also comprises fragments from these groups in which up to 15% of the residues are changed.

In another embodiment, the invention encompasses polypeptides that are naturally occurring allelic variants of the sequence selected from the group consisting of SEQ ID NO:2n, wherein n is an integer between 1 and 174. These allelic variants include amino acid sequences that are the translations of nucleic acid sequences differing by a single nucleotide from nucleic acid sequences selected from the group consisting of SEQ ID NOS: 2n−1, wherein n is an integer between 1 and 174. The variant polypeptide where any amino acid changed in the chosen sequence is changed to provide a conservative substitution.

In another embodiment, the invention comprises a pharmaceutical composition involving a polypeptide with an amino acid sequence selected from the group consisting of SEQ ID NO:2n, wherein n is an integer between 1 and 174 and a pharmaceutically acceptable carrier. In another embodiment, the invention involves a kit, including, in one or more containers, this pharmaceutical composition.

In another embodiment, the invention includes the use of a therapeutic in the manufacture of a medicament for treating a syndrome associated with a human disease, the disease being selected from a pathology associated with a polypeptide with an amino acid sequence selected from the group consisting of SEQ ID NO:2n, wherein n is an integer between 1 and 174 wherein said therapeutic is the polypeptide selected from this group.

In another embodiment, the invention comprises a method for determining the presence or amount of a polypeptide with an amino acid sequence selected from the group consisting of SEQ ID NO:2n, wherein n is an integer between 1 and 174 in a sample, the method involving providing the sample; introducing the sample to an antibody that binds immunospecifically to the polypeptide; and determining the presence or amount of antibody bound to the polypeptide, thereby determining the presence or amount of polypeptide in the sample.

In another embodiment, the invention includes a method for determining the presence of or predisposition to a disease associated with altered levels of a polypeptide with an amino acid sequence selected from the group consisting of SEQ ID NO:2n, wherein n is an integer between 1 and 174 in a first mammalian subject, the method involving measuring the level of expression of the polypeptide in a sample from the first mammalian subject; and comparing the amount of the polypeptide in this sample to the amount of the polypeptide present in a control sample from a second mammalian subject known not to have, or not to be predisposed to, the disease, wherein an alteration in the expression level of the polypeptide in the first subject as compared to the control sample indicates the presence of or predisposition to the disease.

In another embodiment, the invention involves a method of identifying an agent that binds to a polypeptide with an amino acid sequence selected from the group consisting of SEQ ID NO:2n, wherein n is an integer between 1 and 174, the method including introducing the polypeptide to the agent; and determining whether the agent binds to the polypeptide. The agent could be a cellular receptor or a downstream effector.

In another embodiment, the invention involves a method for identifying a potential therapeutic agent for use in treatment of a pathology, wherein the pathology is related to aberrant expression or aberrant physiological interactions of a polypeptide with an amino acid sequence selected from the group consisting of SEQ ID NO:2n, wherein n is an integer between 1 and 174, the method including providing a cell expressing the polypeptide of the invention and having a property or function ascribable to the polypeptide; contacting the cell with a composition comprising a candidate substance; and determining whether the substance alters the property or function ascribable to the polypeptide; whereby, if an alteration observed in the presence of the substance is not observed when the cell is contacted with a composition devoid of the substance, the substance is identified as a potential therapeutic agent.

In another embodiment, the invention involves a method for screening for a modulator of activity or of latency or predisposition to a pathology associated with a polypeptide having an amino acid sequence selected from the group consisting of SEQ ID NO:2n, wherein n is an integer between 1 and 174, the method including administering a test compound to a test animal at increased risk for a pathology associated with the polypeptide of the invention, wherein the test animal recombinantly expresses the polypeptide of the invention; measuring the activity of the polypeptide in the test animal after administering the test compound; and comparing the activity of the protein in the test animal with the activity of the polypeptide in a control animal not administered the polypeptide, wherein a change in the activity of the polypeptide in the test animal relative to the control animal indicates the test compound is a modulator of latency of, or predisposition to, a pathology associated with the polypeptide of the invention. The recombinant test animal could express a test protein transgene or express the transgene under the control of a promoter at an increased level relative to a wild-type test animal the promoter may or may not b the native gene promoter of the transgene.

In another embodiment, the invention involves a method for modulating the activity of a polypeptide with an amino acid sequence selected from the group consisting of SEQ ID NO:2n, wherein n is an integer between 1 and 174, the method including introducing a cell sample expressing the polypeptide with a compound that binds to the polypeptide in an amount sufficient to modulate the activity of the polypeptide.

In another embodiment, the invention involves a method of treating or preventing a pathology associated with a polypeptide with an amino acid sequence selected from the group consisting of SEQ ID NO:2n, wherein n is an integer between 1 and 174, the method including administering the polypeptide to a subject in which such treatment or prevention is desired in an amount sufficient to treat or prevent the pathology in the subject. The subject could be human.

In another embodiment, the invention involves a method of treating a pathological state in a mammal, the method including administering to the mammal a polypeptide in an amount that is sufficient to alleviate the pathological state, wherein the polypeptide is a polypeptide having an amino acid sequence at least 95% identical to a polypeptide having the amino acid sequence selected from the group consisting of SEQ ID NO:2n, wherein n is an integer between 1 and 174 or a biologically active fragment thereof.

In another embodiment, the invention involves an isolated nucleic acid molecule comprising a nucleic acid sequence encoding a polypeptide having an amino acid sequence selected from the group consisting of a mature form of the amino acid sequence given SEQ ID NO:2n, wherein n is an integer between 1 and 174; a variant of a mature form of the amino acid sequence selected from the group consisting of SEQ ID NO:2n, wherein n is an integer between 1 and 174 wherein any amino acid in the mature form of the chosen sequence is changed to a different amino acid, provided that no more than 15% of the amino acid residues in the sequence of the mature form are so changed; the amino acid sequence selected from the group consisting of SEQ ID NO:2n, wherein n is an integer between 1 and 174; a variant of the amino acid sequence selected from the group consisting of SEQ ID NO:2n, wherein n is an integer between 1 and 174, in which any amino acid specified in the chosen sequence is changed to a different amino acid, provided that no more than 15% of the amino acid residues in the sequence are so changed; a nucleic acid fragment encoding at least a portion of a polypeptide comprising the amino acid sequence selected from the group consisting of SEQ ID NO:2n, wherein n is an integer between 1 and 174 or any variant of the polypeptide wherein any amino acid of the chosen sequence is changed to a different amino acid, provided that no more than 10% of the amino acid residues in the sequence are so changed; and the complement of any of the nucleic acid molecules.

In another embodiment, the invention comprises an isolated nucleic acid molecule having a nucleic acid sequence encoding a polypeptide comprising an amino acid sequence selected from the group consisting of a mature form of the amino acid sequence given SEQ ID NO:2n, wherein n is an integer between 1 and 174, wherein the nucleic acid molecule comprises the nucleotide sequence of a naturally occurring allelic nucleic acid variant.

In another embodiment, the invention involves an isolated nucleic acid molecule including a nucleic acid sequence encoding a polypeptide having an amino acid sequence selected from the group consisting of a mature form of the amino acid sequence given SEQ ID NO:2n, wherein n is an integer between 1 and 174 that encodes a variant polypeptide, wherein the variant polypeptide has the polypeptide sequence of a naturally occurring polypeptide variant.

In another embodiment, the invention comprises an isolated nucleic acid molecule having a nucleic acid sequence encoding a polypeptide comprising an amino acid sequence selected from the group consisting of a mature form of the amino acid sequence given SEQ ID NO:2n, wherein n is an integer between 1 and 174, wherein the nucleic acid molecule differs by a single nucleotide from a nucleic acid sequence selected from the group consisting of SEQ ID NOS: 2n−1, wherein n is an integer between 1 and 174.

In another embodiment, the invention includes an isolated nucleic acid molecule having a nucleic acid sequence encoding a polypeptide including an amino acid sequence selected from the group consisting of a mature form of the amino acid sequence given SEQ ID NO:2n, wherein n is an integer between 1 and 174, wherein the nucleic acid molecule comprises a nucleotide sequence selected from the group consisting of the nucleotide sequence selected from the group consisting of SEQ ID NO:2n−1, wherein n is an integer between 1 and 174; a nucleotide sequence wherein one or more nucleotides in the nucleotide sequence selected from the group consisting of SEQ ID NO:2n−1, wherein n is an integer between 1 and 174 is changed from that selected from the group consisting of the chosen sequence to a different nucleotide provided that no more than 15% of the nucleotides are so changed; a nucleic acid fragment of the sequence selected from the group consisting of SEQ ID NO:2n−1, wherein n is an integer between 1 and 174; and a nucleic acid fragment wherein one or more nucleotides in the nucleotide sequence selected from the group consisting of SEQ ID NO:2n−1, wherein n is an integer between 1 and 174 is changed from that selected from the group consisting of the chosen sequence to a different nucleotide provided that no more than 15% of the nucleotides are so changed.

In another embodiment, the invention includes an isolated nucleic acid molecule having a nucleic acid sequence encoding a polypeptide including an amino acid sequence selected from the group consisting of a mature form of the amino acid sequence given SEQ ID NO:2n, wherein n is an integer between 1 and 174, wherein the nucleic acid molecule hybridizes under stringent conditions to the nucleotide sequence selected from the group consisting of SEQ ID NO:2n−1, wherein n is an integer between 1 and 174, or a complement of the nucleotide sequence.

In another embodiment, the invention includes an isolated nucleic acid molecule having a nucleic acid sequence encoding a polypeptide including an amino acid sequence selected from the group consisting of a mature form of the amino acid sequence given SEQ ID NO:2n, wherein n is an integer between 1 and 174, wherein the nucleic acid molecule has a nucleotide sequence in which any nucleotide specified in the coding sequence of the chosen nucleotide sequence is changed from that selected from the group consisting of the chosen sequence to a different nucleotide provided that no more than 15% of the nucleotides in the chosen coding sequence are so changed, an isolated second polynucleotide that is a complement of the first polynucleotide, or a fragment of any of them.

In another embodiment, the invention includes a vector involving the nucleic acid molecule having a nucleic acid sequence encoding a polypeptide including an amino acid sequence selected from the group-consisting of a mature form of the amino acid sequence given SEQ ID NO:2n, wherein n is an integer between 1 and 174. This vector can have a promoter operably linked to the nucleic acid molecule. This vector can be located within a cell.

In another embodiment, the invention involves a method for determining the presence or amount of a nucleic acid molecule having a nucleic acid sequence encoding a polypeptide including an amino acid sequence selected from the group consisting of a mature form of the amino acid sequence given SEQ ID NO:2n, wherein n is an integer between 1 and 174 in a sample, the method including providing the sample; introducing the sample to a probe that binds to the nucleic acid molecule; and determining the presence or amount of the probe bound to the nucleic acid molecule, thereby determining the presence or amount of the nucleic acid molecule in the sample. The presence or amount of the nucleic acid molecule is used as a marker for cell or tissue type. The cell type can be cancerous.

In another embodiment, the invention involves a method for determining the presence of or predisposition for a disease associated with altered levels of a nucleic acid molecule having a nucleic acid sequence encoding a polypeptide including an amino acid sequence selected from the group consisting of a mature form of the amino acid sequence given SEQ ID NO:2n, wherein n is an integer between 1 and 174 in a first mammalian subject, the method including measuring the amount of the nucleic acid in a sample from the first mammalian subject; and comparing the amount of the nucleic acid in the sample of step (a) to the amount of the nucleic acid present in a control sample from a second mammalian subject known not to have or not be predisposed to, the disease; wherein an alteration in the level of the nucleic acid in the first subject as compared to the control sample indicates the presence of or predisposition to the disease.

The invention further provides an antibody that binds immunospecifically to a NOVX polypeptide. The NOVX antibody may be monoclonal, humanized, or a fully human antibody. Preferably, the antibody has a dissociation constant for the binding of the NOVX polypeptide to the antibody less than 1×10⁻⁹ M. More preferably, the NOVX antibody neutralizes the activity of the NOVX polypeptide.

In a further aspect, the invention provides for the use of a therapeutic in the manufacture of a medicament for treating a syndrome associated with a human disease, associated with a NOVX polypeptide. Preferably the therapeutic is a NOVX antibody.

In yet a further aspect, the invention provides a method of treating or preventing a NOVX-associated disorder, a method of treating a pathological state in a mammal, and a method of treating or preventing a pathology associated with a polypeptide by administering a NOVX antibody to a subject in an amount sufficient to treat or prevent the disorder.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the following detailed description and claims.

### BRIEF DESCRIPTION OF THE DRAWINGS

FIG. 1 is a bar diagram showing the activation of 786-0 epithelial cell BrdU incorporation by CG51051-06 protein.

### DETAILED DESCRIPTION OF THE INVENTION

The present invention provides novel nucleotides and polypeptides encoded thereby. Included in the invention are the novel nucleic acid sequences, their encoded polypeptides, antibodies, and other related compounds. The sequences are collectively referred to herein as “NOVX nucleic acids” or “NOVX polynucleotides” and the corresponding encoded polypeptides are referred to as “NOVX polypeptides” or “NOVX proteins.” Unless indicated otherwise, “NOVX” is meant to refer to any of the novel sequences disclosed herein. Table A provides a summary of the NOVX nucleic acids and their encoded polypeptides.

TABLE A
SEQUENCES AND CORRESPONDING SEQ ID NUMBERS

| | | SEQ ID | SEQ ID | |
|---|---|---|---|---|
| | | NO | NO | |
| NOVX | Internal | (nucleic | (amino | |
| Assignment | Identification | acid) | acid) | Homology |
| NOV1a | CG103910-02 | 1 | 2 | Bone morphogenetic protein 7 |
| | | | | (Osteogenic protein 1) - Homo sapiens |
| NOV1b | CG103910-03 | 3 | 4 | Bone morphogenetic protein 7 |
| | | | | (Osteogenic protein 1) - Homo sapiens |
| NOV1c | CG103910-01 | 5 | 6 | Bone morphogenetic protein 7 |
| | | | | (Osteogenic protein 1) - Homo sapiens |
| NOV1d | CG103910-04 | 7 | 8 | Bone morphogenetic protein 7 |
| | | | | (Osteogenic protein 1) - Homo sapiens |
| NOV1e | 11382317 | 9 | 10 | Bone morphogenetic protein 7 |
| | | | | (Osteogenic protein 1) - Homo sapiens |
| NOV2a | CG106298-02 | 11 | 12 | Complement factor H-related protein 4 |
| | | | | precursor (FHR-4) - Homo sapiens |
| NOV2b | CG106298-01 | 13 | 14 | Complement factor H-related protein 4 |
| | | | | precursor (FHR-4) - Homo sapiens |
| NOV3a | CG110590-02 | 15 | 16 | Neuralin precursor (Ventroptin) - Homo |
| | | | | sapiens |
| NOV3b | CG110590-01 | 17 | 18 | Neuralin precursor (Ventroptin) - Homo |
| | | | | sapiens |
| NOV3c | 13382325 | 19 | 20 | Neuralin precursor (Ventroptin) - Homo |
| | | | | sapiens |
| NOV3d | 13382326 | 21 | 22 | Neuralin precursor (Ventroptin) - Homo |
| | | | | sapiens |
| NOV4a | CG114555-01 | 23 | 24 | Solute carrier family 2, facilitated |
| | | | | glucose transporter, member 9 (Glucose |
| | | | | transporter type 9) - Homo sapiens |
| NOV4b | 247847074 | 25 | 26 | Solute carrier family 2, facilitated |
| | | | | glucose transporter, member 9 (Glucose |
| | | | | transporter type 9) - Homo sapiens |
| NOV4c | 247847070 | 27 | 28 | Solute carrier family 2, facilitated |
| | | | | glucose transporter, member 9 (Glucose |
| | | | | transporter type 9) - Homo sapiens |
| NOV4d | 247847055 | 29 | 30 | Solute carrier family 2, facilitated |
| | | | | glucose transporter, member 9 (Glucose |
| | | | | transporter type 9) - Homo sapiens |
| NOV4e | 247847059 | 31 | 32 | Solute carrier family 2, facilitated |
| | | | | glucose transporter, member 9 (Glucose |
| | | | | transporter type 9) - Homo sapiens |
| NOV4f | 247847047 | 33 | 34 | Solute carrier family 2, facilitated |
| | | | | glucose transporter, member 9 (Glucose |
| | | | | transporter type 9) - Homo sapiens |
| NOV4g | CG114555-02 | 35 | 36 | Solute carrier family 2, facilitated |
| | | | | glucose transporter, member 9 (Glucose |
| | | | | transporter type 9) - Homo sapiens |
| NOV4h | CG114555-03 | 37 | 38 | Solute carrier family 2, facilitated |
| | | | | glucose transporter, member 9 (Glucose |
| | | | | transporter type 9) - Homo sapiens |
| NOV4i | CG114555-04 | 39 | 40 | Solute carrier family 2, facilitated |
| | | | | glucose transporter, member 9 (Glucose |
| | | | | transporter type 9) - Homo sapiens |
| NOV4j | 13379365 | 41 | 42 | Solute carrier family 2, facilitated |
| | | | | glucose transporter, member 9 (Glucose |
| | | | | transporter type 9) - Homo sapiens |
| NOV4k | 13379364 | 43 | 44 | Solute carrier family 2, facilitated |
| | | | | glucose transporter, member 9 (Glucose |
| | | | | transporter type 9) - Homo sapiens |
| NOV4l | 13379363 | 45 | 46 | Solute carrier family 2, facilitated |
| | | | | glucose transporter, member 9 (Glucose |
| | | | | transporter type 9) - Homo sapiens |
| NOV4m | 13379362 | 47 | 48 | Solute carrier family 2, facilitated |
| | | | | glucose transporter, member 9 (Glucose |
| | | | | transporter type 9) - Homo sapiens |
| NOV4n | 13379620 | 49 | 50 | Solute carrier family 2, facilitated |
| | | | | glucose transporter, member 9 (Glucose |
| | | | | transporter type 9) - Homo sapiens |
| NOV5a | CG181662-01 | 51 | 52 | Protein farnesyltransferase alpha subunit |
| | | | | (EC 2.5.1.-) (CAAX farnesyltransferase |
| | | | | alpha subunit) (RAS proteins |
| | | | | prenyltransferase alpha) (FTase-alpha) - |
| | | | | Homo sapiens |
| NOV5b | CG181662-02 | 53 | 54 | Protein farnesyltransferase alpha subunit |
| | | | | (EC 2.5.1.-) (CAAX farnesyltransferase |
| | | | | alpha subunit) (RAS proteins |
| | | | | prenyltransferase alpha) (FTase-alpha) - |
| | | | | Homo sapiens |
| NOV5c | 307686795 | 55 | 56 | Protein farnesyltransferase alpha subunit |
| | | | | (EC 2.5.1.-) (CAAX farnesyltransferase |
| | | | | alpha subunit) (RAS proteins |
| | | | | prenyltransferase alpha) (FTase-alpha) - |
| | | | | Homo sapiens |
| NOV5d | CG181662-03 | 57 | 58 | Protein farnesyltransferase alpha subunit |
| | | | | (EC 2.5.1.-) (CAAX farnesyltransferase |
| | | | | alpha subunit) (RAS proteins |
| | | | | prenyltransferase alpha) (FTase-alpha) - |
| | | | | Homo sapiens |
| NOV5e | CG181662-04 | 59 | 60 | Protein farnesyltransferase alpha subunit |
| | | | | (EC 2.5.1.-) (CAAX farnesyltransferase |
| | | | | alpha subunit) (RAS proteins |
| | | | | prenyltransferase alpha) (FTase-alpha) - |
| | | | | Homo sapiens |
| NOV5f | 13382357 | 61 | 62 | Protein farnesyltransferase alpha subunit |
| | | | | (EC 2.5.1.-) (CAAX farnesyltransferase |
| | | | | alpha subunit) (RAS proteins |
| | | | | prenyltransferase alpha) (FTase-alpha) - |
| | | | | Homo sapiens |
| NOV5g | 13377970 | 63 | 64 | Protein farnesyltransferase alpha subunit |
| | | | | (EC 2.5.1.-) (CAAX farnesyltransferase |
| | | | | alpha subunit) (RAS proteins |
| | | | | prenyltransferase alpha) (FTase-alpha) - |
| | | | | Homo sapiens |
| NOV5h | 13378241 | 65 | 66 | Protein farnesyltransferase alpha subunit |
| | | | | (EC 2.5.1.-) (CAAX farnesyltransferase |
| | | | | alpha subunit) (RAS proteins |
| | | | | prenyltransferase alpha) (FTase-alpha) - |
| | | | | Homo sapiens |
| NOV5i | 13377901 | 67 | 68 | Protein farnesyltransferase alpha subunit |
| | | | | (EC 2.5.1.-) (CAAX farnesyltransferase |
| | | | | alpha subunit) (RAS proteins |
| | | | | prenyltransferase alpha) (FTase-alpha) - |
| | | | | Homo sapiens |
| NOV5j | 13377900 | 69 | 70 | Protein farnesyltransferase alpha subunit |
| | | | | (EC 2.5.1.-) (CAAX farnesyltransferase |
| | | | | alpha subunit) (RAS proteins |
| | | | | prenyltransferase alpha) (FTase-alpha) - |
| | | | | Homo sapiens |
| NOV6a | CG182223-01 | 71 | 72 | Human neurotransmission-associated |
| | | | | protein NTRAN8 - Homo sapiens |
| NOV7a | CG183585-01 | 73 | 74 | Adult male liver tumor cDNA, RIKEN |
| | | | | full-length enriched library, |
| | | | | clone: C730027O17 product: hypothetical |
| | | | | protein, full insert sequence - Mus |
| | | | | musculus |
| NOV8a | CG183860-01 | 75 | 76 | Human secreted protein HNTNC20 - |
| | | | | Homo sapiens |
| NOV9a | CG184416-01 | 77 | 78 | MMP-23 (MIFR/FEMALYSIN) |
| | | | | (DJ283E3.2.1) (Matrix metalloproteinase |
| | | | | MMP21/22A (MIFR1)) (Matrix |
| | | | | metalloproteinase 23B) - Homo sapiens |
| NOV10a | CG185200-01 | 79 | 80 | Human secreted/transmembrane protein, |
| | | | | PRO 1377 - Homo sapiens |
| NOV10b | CG185200-02 | 81 | 82 | Human secreted/transmembrane protein, |
| | | | | PRO 1377 - Homo sapiens |
| NOV11a | CG50513-01 | 83 | 84 | central nervous system protein #236 - |
| | | | | Homo sapiens |
| NOV11b | 273654175 | 85 | 86 | central nervous system protein #236 - |
| | | | | Homo sapiens |
| NOV11c | CG50513-02 | 87 | 88 | central nervous system protein #236 - |
| | | | | Homo sapiens |
| NOV11d | CG50513-03 | 89 | 90 | central nervous system protein #236 - |
| | | | | Homo sapiens |
| NOV11e | CG50513-04 | 91 | 92 | central nervous system protein #236 - |
| | | | | Homo sapiens |
| NOV11f | CG50513-05 | 93 | 94 | central nervous system protein #236 - |
| | | | | Homo sapiens |
| NOV11g | CG50513-06 | 95 | 96 | central nervous system protein #236 - |
| | | | | Homo sapiens |
| NOV11h | CG50513-07 | 97 | 98 | central nervous system protein #236 - |
| | | | | Homo sapiens |
| NOV11i | 13376798 | 99 | 100 | central nervous system protein #236 - |
| | | | | Homo sapiens |
| NOV11j | 13376799 | 101 | 102 | central nervous system protein #236 - |
| | | | | Homo sapiens |
| NOV12a | CG50949-03 | 103 | 104 | Membrane-type mosaic serine protease - |
| | | | | Homo sapiens |
| NOV12b | 197192399 | 105 | 106 | Membrane-type mosaic serine protease - |
| | | | | Homo sapiens |
| NOV12c | 257499999 | 107 | 108 | Membrane-type mosaic serine protease - |
| | | | | Homo sapiens |
| NOV12d | 257450010 | 109 | 110 | Membrane-type mosaic serine protease - |
| | | | | Homo sapiens |
| NOV12e | 252417780 | 111 | 112 | Membrane-type mosaic serine protease - |
| | | | | Homo sapiens |
| NOV12f | 252417791 | 113 | 114 | Membrane-type mosaic serine protease - |
| | | | | Homo sapiens |
| NOV12g | 252417821 | 115 | 116 | Membrane-type mosaic serine protease - |
| | | | | Homo sapiens |
| NOV12h | 252417840 | 117 | 118 | Membrane-type mosaic serine protease - |
| | | | | Homo sapiens |
| NOV12i | 257474313 | 119 | 120 | Membrane-type mosaic serine protease - |
| | | | | Homo sapiens |
| NOV12j | 257474324 | 121 | 122 | Membrane-type mosaic serine protease - |
| | | | | Homo sapiens |
| NOV12k | CG50949-06 | 123 | 124 | Membrane-type mosaic serine protease - |
| | | | | Homo sapiens |
| NOV12l | 268669017 | 125 | 126 | Membrane-type mosaic serine protease - |
| | | | | Homo sapiens |
| NOV12m | CG50949-05 | 127 | 128 | Membrane-type mosaic serine protease - |
| | | | | Homo sapiens |
| NOV12n | 317431859 | 129 | 130 | Membrane-type mosaic serine protease - |
| | | | | Homo sapiens |
| NOV12o | CG50949-01 | 131 | 132 | Membrane-type mosaic serine protease - |
| | | | | Homo sapiens |
| NOV12p | CG50949-02 | 133 | 134 | Membrane-type mosaic serine protease - |
| | | | | Homo sapiens |
| NOV12q | CG50949-04 | 135 | 136 | Membrane-type mosaic serine protease - |
| | | | | Homo sapiens |
| NOV12r | CG50949-07 | 137 | 138 | Membrane-type mosaic serine protease - |
| | | | | Homo sapiens |
| NOV12s | 13374729 | 139 | 140 | Membrane-type mosaic serine protease - |
| | | | | Homo sapiens |
| NOV12t | 13374730 | 141 | 142 | Membrane-type mosaic serine protease - |
| | | | | Homo sapiens |
| NOV12u | 13374731 | 143 | 144 | Membrane-type mosaic serine protease - |
| | | | | Homo sapiens |
| NOV13a | CG51018-01 | 145 | 146 | Matrilin-2 precursor - Homo sapiens |
| NOV13b | 274051273 | 147 | 148 | Matrilin-2 precursor - Homo sapiens |
| NOV13c | 274051251 | 149 | 150 | Matrilin-2 precursor - Homo sapiens |
| NOV13d | 274051253 | 151 | 152 | Matrilin-2 precursor - Homo sapiens |
| NOV13e | 306562753 | 153 | 154 | Matrilin-2 precursor - Homo sapiens |
| NOV13f | CG51018-02 | 155 | 156 | Matrilin-2 precursor - Homo sapiens |
| NOV13g | CG51018-03 | 157 | 158 | Matrilin-2 precursor - Homo sapiens |
| NOV13h | 13374217 | 159 | 160 | Matrilin-2 precursor - Homo sapiens |
| NOV14a | CG51051-07 | 161 | 162 | Netrin-G1d - Mus musculus |
| NOV14b | CG51051-14 | 163 | 164 | Netrin-G1d - Mus musculus |
| NOV14c | 254537195 | 165 | 166 | Netrin-G1d - Mus musculus |
| NOV14d | 254537282 | 167 | 168 | Netrin-G1d - Mus musculus |
| NOV14e | CG51051-09 | 169 | 170 | Netrin-G1d - Mus musculus |
| NOV14f | 304965116 | 171 | 172 | Netrin-G1d - Mus musculus |
| NOV14g | 273711018 | 173 | 174 | Netrin-G1d - Mus musculus |
| NOV14h | 273711053 | 175 | 176 | Netrin-G1d - Mus musculus |
| NOV14i | 274051275 | 177 | 178 | Netrin-G1d - Mus musculus |
| NOV14j | CG51051-01 | 179 | 180 | Netrin-G1d - Mus musculus |
| NOV14k | CG51051-02 | 181 | 182 | Netrin-G1d - Mus musculus |
| NOV141 | CG51051-03 | 183 | 184 | Netrin-G1d - Mus musculus |
| NOV14m | CG51051-04 | 185 | 186 | Netrin-G1d - Mus musculus |
| NOV14n | CG51051-05 | 187 | 188 | Netrin-G1d - Mus musculus |
| NOV14o | CG51051-06 | 189 | 190 | Netrin-G1d - Mus musculus |
| NOV14p | CG51051-08 | 191 | 192 | Netrin-G1d - Mus musculus |
| NOV14q | CG51051-10 | 193 | 194 | Netrin-G1d - Mus musculus |
| NOV14r | CG51051-11 | 195 | 196 | Netrin-G1d - Mus musculus |
| NOV14s | CG51051-12 | 197 | 198 | Netrin-G1d - Mus musculus |
| NOV14t | CG51051-13 | 199 | 200 | Netrin-G1d - Mus musculus |
| NOV14u | CG51051-15 | 201 | 202 | Netrin-G1d - Mus musculus |
| NOV14v | CG51051-16 | 203 | 204 | Netrin-G1d - Mus musculus |
| NOV14w | 13380736 | 205 | 206 | Netrin-G1d - Mus musculus |
| NOV14x | 13380734 | 207 | 208 | Netrin-G1d - Mus musculus |
| NOV14y | 13382329 | 209 | 210 | Netrin-G1d - Mus musculus |
| NOV15a | CG52261-01 | 211 | 212 | Netrin-G1d - Mus musculus |
| NOV15b | 268667469 | 213 | 214 | Netrin-G1d - Mus musculus |
| NOV15c | CG52261-02 | 215 | 216 | Netrin-G1d - Mus musculus |
| NOV15d | 13382342 | 217 | 218 | Netrin-G1d - Mus musculus |
| NOV15e | 13382341 | 219 | 220 | Netrin-G1d - Mus musculus |
| NOV16a | CG52414-02 | 221 | 222 | Epidermal growth factor receptor-related |
| | | | | protein homolog - Mus musculus |
| NOV16b | 305262879 | 223 | 224 | Epidermal growth factor receptor-related |
| | | | | protein homolog - Mus musculus |
| NOV16c | 319073326 | 225 | 226 | Epidermal growth factor receptor-related |
| | | | | protein homolog - Mus musculus |
| NOV16d | CG52414-01 | 227 | 228 | Epidermal growth factor receptor-related |
| | | | | protein homolog - Mus musculus |
| NOV16e | CG52414-03 | 229 | 230 | Epidermal growth factor receptor-related |
| | | | | protein homolog - Mus musculus |
| NOV16f | 13379509 | 231 | 232 | Epidermal growth factor receptor-related |
| | | | | protein homolog - Mus musculus |
| NOV16g | 13381817 | 233 | 234 | Epidermal growth factor receptor-related |
| | | | | protein homolog - Mus musculus |
| NOV16h | 13382069 | 235 | 236 | Epidermal growth factor receptor-related |
| | | | | protein homolog - Mus musculus |
| NOV16i | 13381560 | 237 | 238 | Epidermal growth factor receptor-related |
| | | | | protein homolog - Mus musculus |
| NOV17a | CG52643-02 | 239 | 240 | Human follistatin-related protein |
| NOV17b | 259341359 | 241 | 242 | Human follistatin-related protein |
| NOV17c | 268824728 | 243 | 244 | Human follistatin-related protein |
| NOV17d | 268825987 | 245 | 246 | Human follistatin-related protein |
| NOV17e | 268825997 | 247 | 248 | Human follistatin-related protein |
| NOV17f | 275698334 | 249 | 250 | Human follistatin-related protein |
| NOV17g | CG52643-04 | 251 | 252 | Human follistatin-related protein |
| NOV17h | 301380586 | 253 | 254 | Human follistatin-related protein |
| NOV17i | 289087852 | 255 | 256 | Human follistatin-related protein |
| NOV17j | 289081920 | 257 | 258 | Human follistatin-related protein |
| NOV17k | 289098038 | 259 | 260 | Human follistatin-related protein |
| NOV17l | 311060818 | 261 | 262 | Human follistatin-related protein |
| NOV17m | 311885703 | 263 | 264 | Human follistatin-related protein |
| NOV17n | CG52643-01 | 265 | 266 | Human follistatin-related protein |
| NOV17o | CG52643-03 | 267 | 268 | Human follistatin-related protein |
| NOV17p | CG52643-05 | 269 | 270 | Human follistatin-related protein |
| NOV17q | CG52643-06 | 271 | 272 | Human follistatin-related protein |
| NOV17r | 13382322 | 273 | 274 | Human follistatin-related protein |
| NOV17s | 13382324 | 275 | 276 | Human follistatin-related protein |
| NOV17t | 13381678 | 277 | 278 | Human follistatin-related protein |
| NOV18a | CG53270-01 | 279 | 280 | Serine/threonine kinase FKSG81 (Testis- |
| | | | | specific serine/threonine kinase 1) - |
| | | | | Homo sapiens |
| NOV18b | 274089779 | 281 | 282 | Serine/threonine kinase FKSG81 (Testis- |
| | | | | specific serine/threonine kinase 1) - |
| | | | | Homo sapiens |
| NOV18c | CG53270-02 | 283 | 284 | Serine/threonine kinase FKSG81 (Testis- |
| | | | | specific serine/threonine kinase 1) - |
| | | | | Homo sapiens |
| NOV18d | 13382344 | 285 | 286 | Serine/threonine kinase FKSG81 (Testis- |
| | | | | specific serine/threonine kinase 1) - |
| | | | | Homo sapiens |
| NOV18e | 13382345 | 287 | 288 | Serine/threonine kinase FKSG81 (Testis- |
| | | | | specific serine/threonine kinase 1) - |
| | | | | Homo sapiens |
| NOV18f | 13376391 | 289 | 290 | Serine/threonine kinase FKSG81 (Testis- |
| | | | | specific serine/threonine kinase 1) - |
| | | | | Homo sapiens |
| NOV18g | 13376390 | 291 | 292 | Serine/threonine kinase FKSG81 (Testis- |
| | | | | specific serine/threonine kinase 1) - |
| | | | | Homo sapiens |
| NOV18h | 13376389 | 293 | 294 | Serine/threonine kinase FKSG81 (Testis- |
| | | | | specific serine/threonine kinase 1) - |
| | | | | Homo sapiens |
| NOV19a | CG54254-04 | 295 | 296 | Fibronectin leucine rich transmembrane |
| | | | | protein |
| NOV19b | 247846813 | 297 | 298 | Fibronectin leucine rich transmembrane |
| | | | | protein |
| NOV19c | 247846825 | 299 | 300 | Fibronectin leucine rich transmembrane |
| | | | | protein |
| NOV19d | 247846967 | 301 | 302 | Fibronectin leucine rich transmembrane |
| | | | | protein |
| NOV19e | 283841186 | 303 | 304 | Fibronectin leucine rich transmembrane |
| | | | | protein |
| NOV19f | CG54254-01 | 305 | 306 | Fibronectin leucine rich transmembrane |
| | | | | protein |
| NOV19g | CG54254-02 | 307 | 308 | Fibronectin leucine rich transmembrane |
| | | | | protein |
| NOV19h | CG54254-03 | 309 | 310 | Fibronectin leucine rich transmembrane |
| | | | | protein |
| NOV19i | CG54254-05 | 311 | 312 | Fibronectin leucine rich transmembrane |
| | | | | protein |
| NOV19j | CG54254-06 | 313 | 314 | Fibronectin leucine rich transmembrane |
| | | | | protein |
| NOV19k | CG54254-07 | 315 | 316 | Fibronectin leucine rich transmembrane |
| | | | | protein |
| NOV19l | 13375078 | 317 | 318 | Fibronectin leucine rich transmembrane |
| | | | | protein |
| NOV19m | 13376406 | 319 | 320 | Fibronectin leucine rich transmembrane |
| | | | | protein |
| NOV19n | 13375079 | 321 | 322 | Fibronectin leucine rich transmembrane |
| | | | | protein |
| NOV19o | 13376405 | 323 | 324 | Fibronectin leucine rich transmembrane |
| | | | | protein |
| NOV20a | CG96778-02 | 325 | 326 | Acyl-CoA dehydrogenase, medium-chain |
| | | | | specific, mitochondrial precursor (EC |
| | | | | 1.3.99.3) (MCAD) - Homo sapiens |
| NOV20b | CG96778-01 | 327 | 328 | Acyl-CoA dehydrogenase, medium-chain |
| | | | | specific, mitochondrial precursor (EC |
| | | | | 1.3.99.3) (MCAD) - Homo sapiens |
| NOV20c | 276657466 | 329 | 330 | Acyl-CoA dehydrogenase, medium-chain |
| | | | | specific, mitochondrial precursor (EC |
| | | | | 1.3.99.3) (MCAD) - Homo sapiens |
| NOV20d | 276657530 | 331 | 332 | Acyl-CoA dehydrogenase, medium-chain |
| | | | | specific, mitochondrial precursor (EC |
| | | | | 1.3.99.3) (MCAD) - Homo sapiens |
| NOV20e | 276657538 | 333 | 334 | Acyl-CoA dehydrogenase, medium-chain |
| | | | | specific, mitochondrial precursor (EC |
| | | | | 1.3.99.3) (MCAD) - Homo sapiens |
| NOV20f | 276657616 | 335 | 336 | Acyl-CoA dehydrogenase, medium-chain |
| | | | | specific, mitochondrial precursor (EC |
| | | | | 1.3.99.3) (MCAD) - Homo sapiens |
| NOV20g | CG96778-03 | 337 | 338 | Acyl-CoA dehydrogenase, medium-chain |
| | | | | specific, mitochondrial precursor (EC |
| | | | | 1.3.99.3) (MCAD) - Homo sapiens |
| NOV20h | 13382351 | 339 | 340 | Acyl-CoA dehydrogenase, medium-chain |
| | | | | specific, mitochondrial precursor (EC |
| | | | | 1.3.99.3) (MCAD) - Homo sapiens |
| NOV20i | 13382352 | 341 | 342 | Acyl-CoA dehydrogenase, medium-chain |
| | | | | specific, mitochondrial precursor (EC |
| | | | | 1.3.99.3) (MCAD) - Homo sapiens |
| NOV20J | 13382353 | 343 | 344 | Acyl-CoA dehydrogenase, medium-chain |
| | | | | specific, mitochondrial precursor (EC |
| | | | | 1.3.99.3) (MCAD) - Homo sapiens |
| NOV20k | 13382354 | 345 | 346 | Acyl-CoA dehydrogenase, medium-chain |
| | | | | specific, mitochondrial precursor (EC |
| | | | | 1.3.99.3) (MCAD) - Homo sapiens |
| NOV20l | 12252113 | 347 | 348 | Acyl-CoA dehydrogenase, medium-chain |
| | | | | specific, mitochondrial precursor (EC |
| | | | | 1.3.99.3) (MCAD) - Homo sapiens |

Table A indicates the homology of NOVX polypeptides to known protein families. Thus, the nucleic acids and polypeptides, antibodies and related compounds according to the invention corresponding to a NOVX as identified in column 1 of Table A will be useful in therapeutic and diagnostic applications implicated in, for example, pathologies and disorders associated with the known protein families identified in column 5 of Table A.

Pathologies, diseases, disorders and condition and the like that are associated with NOVX sequences include, but are not limited to: e.g., cardiomyopathy, atherosclerosis, hypertension, congenital heart defects, aortic stenosis, atrial septal defect (ASD), vascular calcification, fibrosis, atrioventricular (A-V) canal defect, ductus arteriosus, pulmonary stenosis, subaortic stenosis, ventricular septal defect (VSD), valve diseases, tuberous sclerosis, scleroderma, obesity, metabolic disturbances associated with obesity, transplantation, osteoarthritis, rheumatoid arthritis, osteochondrodysplasia, adrenoleukodystrophy, congenital adrenal hyperplasia, prostate cancer, diabetes, metabolic disorders, neoplasm; adenocarcinoma, lymphoma, uterus cancer, fertility, glomerulonephritis, hemophilia, hypercoagulation, idiopathic thrombocytopenic purpura, immunodeficiencies, psoriasis, skin disorders, graft versus host disease, AIDS, bronchial asthma, lupus, Crohn's disease; inflammatory bowel disease, ulcerative colitis, multiple sclerosis, treatment of Albright Hereditary Ostocodystrophy, infectious disease, anorexia, cancer-associated cachexia, cancer, neurodegenerative disorders, Alzheimer's Disease, Parkinson's Disorder, immune disorders, hematopoietic disorders, and the various dyslipidemias, schizophrenia, depression, asthma, emphysema, allergies, the metabolic syndrome X and wasting disorders associated with chronic diseases and various cancers, as well as conditions such as transplantation, neuroprotection, fertility, or regeneration (in vitro and in vivo).

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

Consistent with other known members of the family of proteins, identified in column 5 of Table A, the NOVX polypeptides of the present invention show homology to, and contain domains that are characteristic of, other members of such protein families. Details of the sequence relatedness and domain analysis for each NOVX are presented in Example A.

The NOVX nucleic acids and polypeptides can also be used to screen for molecules, which inhibit or enhance NOVX activity or function. Specifically, the nucleic acids and polypeptides according to the invention may be used as targets for the identification of small molecules that modulate or inhibit diseases associated with the protein families listed in Table A.

The NOVX nucleic acids and polypeptides are also useful for detecting specific cell types. Details of the expression analysis for each NOVX are presented in Example C. Accordingly, the NOVX nucleic acids, polypeptides, antibodies and related compounds according to the invention will have diagnostic and therapeutic applications in the detection of a variety of diseases with differential expression in normal vs. diseased tissues, e.g. detection of a variety of cancers.

Additional utilities for NOVX nucleic acids and polypeptides according to the invention are disclosed herein.

NOVX Clones

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

The NOVX genes and their corresponding encoded proteins are useful for preventing, treating or ameliorating medical conditions, e.g., by protein or gene therapy. Pathological conditions can be diagnosed by determining the amount of the new protein in a sample or by determining the presence of mutations in the new genes. Specific uses are described for each of the NOVX genes, based on the tissues in which they are most highly expressed. Uses include developing products for the diagnosis or treatment of a variety of diseases and disorders.

The NOVX nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) a biological defense weapon.

In one specific embodiment, the invention includes an isolated polypeptide comprising an amino acid sequence selected from the group consisting of: (a) a mature form of the amino acid sequence selected from the group consisting of SEQ ID NO: 2n, wherein n is an integer between 1 and 174; (b) a variant of a mature form of the amino acid sequence selected from the group consisting of SEQ ID NO: 2n, wherein n is an integer between 1 and 174, wherein any amino acid in the mature form is changed to a different amino acid, provided that no more than 15% of the amino acid residues in the sequence of the mature form are so changed; (c) an amino acid sequence selected from the group consisting of SEQ ID NO: 2n, wherein n is an integer between 1 and 174; (d) a variant of the amino acid sequence selected from the group consisting of SEQ ID NO:2n, wherein n is an integer between 1 and 174 wherein any amino acid specified in the chosen sequence is changed to a different amino acid, provided that no more than 15% of the amino acid residues in the sequence are so changed; and (e) a fragment of any of (a) through (d).

In another specific embodiment, the invention includes an isolated nucleic acid molecule comprising a nucleic acid sequence encoding a polypeptide comprising an amino acid sequence selected from the group consisting of: (a) a mature form of the amino acid sequence given SEQ ID NO: 2n, wherein n is an integer between 1 and 174; (b) a variant of a mature form of the amino acid sequence selected from the group consisting of SEQ ID NO: 2n, wherein n is an integer between 1 and 174 wherein any amino acid in the mature form of the chosen sequence is changed to a different amino acid, provided that no more than 15% of the amino acid residues in the sequence of the mature form are so changed; (c) the amino acid sequence selected from the group consisting of SEQ ID NO: 2n, wherein n is an integer between 1 and 174; (d) a variant of the amino acid sequence selected from the group consisting of SEQ ID NO: 2n, wherein n is an integer between 1 and 174, in which any amino acid specified in the chosen sequence is changed to a different amino acid, provided that no more than 15% of the amino acid residues in the sequence are so changed; (e) a nucleic acid fragment encoding at least a portion of a polypeptide comprising the amino acid sequence selected from the group consisting of SEQ ID NO: 2n, wherein n is an integer between 1 and 174 or any variant of said polypeptide wherein any amino acid of the chosen sequence is changed to a different amino acid, provided that no more than 10% of the amino acid residues in the sequence are so changed; and (f) the complement of any of said nucleic acid molecules.

In yet another specific embodiment, the invention includes an isolated nucleic acid molecule, wherein said nucleic acid molecule comprises a nucleotide sequence selected from the group consisting of: (a) the nucleotide sequence selected from the group consisting of SEQ ID NO: 2n−1, wherein n is an integer between 1 and 174; (b) a nucleotide sequence wherein one or more nucleotides in the nucleotide sequence selected from the group consisting of SEQ ID NO: 2n−1, wherein n is an integer between 1 and 174 is changed from that selected from the group consisting of the chosen sequence to a different nucleotide provided that no more than 15% of the nucleotides are so changed; (c) a nucleic acid fragment of the sequence selected from the group consisting of SEQ ID NO: 2n−1, wherein n is an integer between 1 and 174; and (d) a nucleic acid fragment wherein one or more nucleotides in the nucleotide sequence selected from the group consisting of SEQ ID NO: 2n−1, wherein n is an integer between 1 and 174 is changed from that selected from the group consisting of the chosen sequence to a different nucleotide provided that no more than 15% of the nucleotides are so changed.

NOVX Nucleic Acids and Polypeptides

One aspect of the invention pertains to isolated nucleic acid molecules that encode NOVX polypeptides or biologically active portions thereof. Also included in the invention are nucleic acid fragments sufficient for use as hybridization probes to identify NOVX-encoding nucleic acids (e.g., NOVX mRNAs) and fragments for use as PCR primers for the amplification and/or mutation of NOVX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

A NOVX nucleic acid can encode a mature NOVX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product encoded by the corresponding gene. Alternatively, it may be defined as the polypeptide, precursor or proprotein encoded by an ORF described herein. The product “mature” form arises, by way of nonlimiting example, as a result of one or more naturally occurring processing steps that may take place within the cell (e.g., host cell) in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristylation or phosphorylation. In general, a mature polypeptide or protein may result from the operation of only one of these processes, or a combination of any of them.

The term “probe”, as utilized herein, refers to nucleic acid sequences of variable length, preferably between at least about 10 nucleotides (nt), about 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single-stranded or double-stranded and designed to have specificity in PCR, membrane-based hybridization technologies, or ELISA-like technologies.

The term “isolated” nucleic acid molecule, as used herein, is a nucleic acid that is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated NOVX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material, or culture medium, or of chemical precursors or other chemicals.

A nucleic acid molecule of the invention, e.g., a nucleic acid molecule having the nucleotide sequence of SEQ ID NO:2n−1, wherein n is an integer between 1 and 174, or a complement of this nucleotide sequence, can be isolated using standard molecular biology techniques and the sequence information provided herein. Using all or a portion of the nucleic acid sequence of SEQ ID NO:2n−1, wherein n is an integer between 1 and 174, as a hybridization probe, NOVX molecules can be isolated using standard hybridization and cloning techniques (e.g., as described in Sambrook, et al., (eds.), MOLECULAR CLONING: A LABORATORY MANUAL 2^{nd}., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template with appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, e.g., using an automated DNA synthesizer.

As used herein, the term “oligonucleotide” refers to a series of linked nucleotide residues. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides of SEQ ID NO:2n−1, wherein n is an integer between 1 and 174, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NO:2n−1, wherein n is an integer between 1 and 174, or a portion of this nucleotide sequence (e.g., a fragment that can be used as a probe or primer or a fragment encoding a biologically-active portion of a NOVX polypeptide). A nucleic acid molecule that is complementary to the nucleotide sequence of SEQ ID NO:2n−1, wherein n is an integer between 1 and 174, is one that is sufficiently complementary to the nucleotide sequence of SEQ ID NO:2n−1, wherein n is an integer between 1 and 174, that it can hydrogen bond with few or no mismatches to the nucleotide sequence shown in SEQ ID NO:2n−1, wherein n is an integer between 1 and 174, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

A “fragment” provided herein is defined as a sequence of at least 6 (contiguous) nucleic acids or at least 4 (contiguous) amino acids, a length sufficient to allow for specific hybridization in the case of nucleic acids or for specific recognition of an epitope in the case of amino acids, and is at most some portion less than a full length sequence. Fragments may be derived from any contiguous portion of a nucleic acid or amino acid sequence of choice.

A full-length NOVX clone is identified as containing an ATG translation start codon and an in-frame stop codon. Any disclosed NOVX nucleotide sequence lacking an ATG start codon therefore encodes a truncated C-terminal fragment of the respective NOVX polypeptide, and requires that the corresponding full-length cDNA extend in the 5′ direction of the disclosed sequence. Any disclosed NOVX nucleotide sequence lacking an in-frame stop codon similarly encodes a truncated N-terminal fragment of the respective NOVX polypeptide, and requires that the corresponding full-length cDNA extend in the 3′ direction of the disclosed sequence.

A “derivative” is a nucleic acid sequence or amino acid sequence formed from the native compounds either directly, by modification or partial substitution. An “analog” is a nucleic acid sequence or amino acid sequence that has a structure similar to, but not identical to, the native compound, e.g. they differs from it in respect to certain components or side chains. Analogs may be synthetic or derived from a different evolutionary origin and may have a similar or opposite metabolic activity compared to wild type. A “homolog” is a nucleic acid sequence or amino acid sequence of a particular gene that is derived from different species.

Derivatives and analogs may be full length or other than full length. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences include those sequences coding for isoforms of NOVX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for a NOVX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human NOVX protein. Homologous nucleic acid sequences include those nucleic acid sequences that encode conservative amino acid substitutions (see below) in SEQ ID NO:2n−1 wherein n is an integer between 1 and 174, as well as a polypeptide possessing NOVX biological activity. Various biological activities of the NOVX proteins are described below.

A NOVX polypeptide is encoded by the open reading frame (“ORF”) of a NOVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bonafide cellular protein, a minimum size requirement is often set, e.g., a stretch of DNA that would encode a protein of 50 amino acids or more.

The nucleotide sequences determined from the cloning of the human NOVX genes allows for the generation of probes and primers designed for use in identifying and/or cloning NOVX homologues in other cell types, e.g. from other tissues, as well as NOVX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence of SEQ ID NO:2n−1, wherein n is an integer between 1 and 174; or an anti-sense strand nucleotide sequence of SEQ ID NO:2n−1, wherein n is an integer between 1 and 174; or of a naturally occurring mutant of SEQ ID NO:2n−1, wherein n is an integer between 1 and 174.

Probes based on the human NOVX nucleotide sequences can be used to detect transcripts or genomic sequences encoding the same or homologous proteins. In various embodiments, the probe has a detectable label attached, e.g. the label can be a radioisotope, a fluorescent compound, an enzyme, or an enzyme co-factor. Such probes can be used as a part of a diagnostic test kit for identifying cells or tissues which mis-express a NOVX protein, such as by measuring a level of a NOVX-encoding nucleic acid in a sample of cells from a subject e.g., detecting NOVX mRNA levels or determining whether a genomic NOVX gene has been mutated or deleted.

“A polypeptide having a biologically-active portion of a NOVX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of NOVX” can be prepared by isolating a portion of SEQ ID NO:2n−1, wherein n is an integer between 1 and 174, that encodes a polypeptide having a NOVX biological activity (the biological activities of the NOVX proteins are described below), expressing the encoded portion of NOVX protein (e.g., by recombinant expression in vitro) and assessing the activity of the encoded portion of NOVX.

NOVX Single Nucleotide Polymorphisms

Variant sequences are also included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, when a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern. Examples include alteration in temporal expression, physiological response regulation, cell type expression regulation, intensity of expression, and stability of transcribed message.

SeqCalling assemblies produced by the exon linking process were selected and extended using the following criteria. Genomic clones having regions with 98% identity to all or part of the initial or extended sequence were identified by BLASTN searches using the relevant sequence to query human genomic databases. The genomic clones that resulted were selected for further analysis because this identity indicates that these clones contain the genomic locus for these SeqCalling assemblies. These sequences were analyzed for putative coding regions as well as for similarity to the known DNA and protein sequences. Programs used for these analyses include Grail, Genscan, BLAST, HMMER, FASTA, Hybrid and other relevant programs.

Some additional genomic regions may have also been identified because selected SeqCalling assemblies map to those regions. Such SeqCalling sequences may have overlapped with regions defined by homology or exon prediction. They may also be included because the location of the fragment was in the vicinity of genomic regions identified by similarity or exon prediction that had been included in the original predicted sequence. The sequence so identified was manually assembled and then may have been extended using one or more additional sequences taken from CuraGen Corporation's human SeqCalling database. SeqCalling fragments suitable for inclusion were identified by the CuraTools™ program SeqExtend or by identifying SeqCalling fragments mapping to the appropriate regions of the genomic clones analyzed.

The regions defined by the procedures described above were then manually integrated and corrected for apparent inconsistencies that may have arisen, for example, from miscalled bases in the original fragments or from discrepancies between predicted exon junctions, EST locations and regions of sequence similarity, to derive the final sequence disclosed herein. When necessary, the process to identify and analyze SeqCalling assemblies and genomic clones was reiterated to derive the full length sequence (Alderborn et al., Determination of Single Nucleotide Polymorphisms by Real-time Pyrophosphate DNA Sequencing. Genome Research. 10 (8) 1249-1265, 2000).

Variants are reported individually but any combination of all or a select subset of variants are also included as contemplated NOVX embodiments of the invention.

NOVX Nucleic Acid and Polypeptide Variants

The invention further encompasses nucleic acid molecules that differ from the nucleotide sequences of SEQ ID NO:2n−1, wherein n is an integer between 1 and 174, due to degeneracy of the genetic code and thus encode the same NOVX proteins as that encoded by the nucleotide sequences of SEQ ID NO:2n−1, wherein n is an integer between 1 and 174. In another embodiment, an isolated nucleic acid molecule of the invention has a nucleotide sequence encoding a protein having an amino acid sequence of SEQ ID NO:2n, wherein n is an integer between 1 and 174.

In addition to the human NOVX nucleotide sequences of SEQ ID NO:2n−1, wherein n is an integer between 1 and 174, it will be appreciated by those skilled in the art that DNA sequence polymorphisms that lead to changes in the amino acid sequences of the NOVX polypeptides may exist within a population (e.g., the human population). Such genetic polymorphism in the NOVX genes may exist among individuals within a population due to natural allelic variation. As used herein, the terms “gene” and “recombinant gene” refer to nucleic acid molecules comprising an open reading frame (ORF) encoding a NOVX protein, preferably a vertebrate NOVX protein. Such natural allelic variations can typically result in 1-5% variance in the nucleotide sequence of the NOVX genes. Any and all such nucleotide variations and resulting amino acid polymorphisms in the NOVX polypeptides, which are the result of natural allelic variation and that do not alter the functional activity of the NOVX polypeptides, are intended to be within the scope of the invention.

Moreover, nucleic acid molecules encoding NOVX proteins from other species, and thus that have a nucleotide sequence that differs from a human SEQ ID NO:2n−1, wherein n is an integer between 1 and 174, are intended to be within the scope of the invention. Nucleic acid molecules corresponding to natural allelic variants and homologues of the NOVX cDNAs of the invention can be isolated based on their homology to the human NOVX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NO:2n−1, wherein n is an integer between 1 and 174. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least about 65% homologous to each other typically remain hybridized to each other.

Homologs (i.e., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to a sequence of SEQ ID NO:2n−1, wherein n is an integer between 1 and 174, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NO:2n−1, wherein n is an integer between 1 and 174, or fragments, analogs or derivatives thereof, under conditions of moderate stringency is provided. A non-limiting example of moderate stringency hybridization conditions are hybridization in 6×SSC, 5× Reinhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art. See, e.g., Ausubel, et al. (eds.), 1993, CURRENT PROTOCOLS IN MOLECULAR BIOLOGY, John Wiley & Sons, NY, and Krieger, 1990; GENE TRANSFER AND EXPRESSION, A LABORATORY MANUAL, Stockton Press, NY.

In a third embodiment, a nucleic acid that is hybridizable to the nucleic acid molecule comprising the nucleotide sequences of SEQ ID NO:2n−1, wherein n is an integer between 1 and 174, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981. Proc Natl Acad Sci USA 78: 6789-6792.

Conservative Mutations

In addition to naturally-occurring allelic variants of NOVX sequences that may exist in the population, the skilled artisan will further appreciate that changes can be introduced by mutation into the nucleotide sequences of SEQ ID NO:2n−1, wherein n is an integer between 1 and 174, thereby leading to changes in the amino acid sequences of the encoded NOVX protein, without altering the functional ability of that NOVX protein. For example, nucleotide substitutions leading to amino acid substitutions at “non-essential” amino acid residues can be made in the sequence of SEQ ID NO:2n, wherein n is an integer between 1 and 174. A “non-essential” amino acid residue is a residue that can be altered from the wild-type sequences of the NOVX proteins without altering their biological activity, whereas an “essential” amino acid residue is required for such biological activity. For example, amino acid residues that are conserved among the NOVX proteins of the invention are predicted to be particularly non-amenable to alteration. Amino acids for which conservative substitutions can be made are well-known within the art.

Another aspect of the invention pertains to nucleic acid molecules encoding NOVX proteins that contain changes in amino acid residues that are not essential for activity. Such NOVX proteins differ in amino acid sequence from SEQ ID NO:2n−1, wherein n is an integer between 1 and 174, yet retain biological activity. In one embodiment, the isolated nucleic acid molecule comprises a nucleotide sequence encoding a protein, wherein the protein comprises an amino acid sequence at least about 40% homologous to the amino acid sequences of SEQ ID NO:2n, wherein n is an integer between 1 and 174. Preferably, the protein encoded by the nucleic acid molecule is at least about 60% homologous to SEQ ID NO:2n, wherein n is an integer between 1 and 174; more preferably at least about 70% homologous to SEQ ID NO:2n, wherein n is an integer between 1 and 174; still more preferably at least about 80% homologous to SEQ ID NO:2n, wherein n is an integer between 1 and 174; even more preferably at least about 90% homologous to SEQ ID NO:2n, wherein n is an integer between 1 and 174; and most preferably at least about 95% homologous to SEQ ID NO:2n, wherein n is an integer between 1 and 174.

An isolated nucleic acid molecule encoding a NOVX protein homologous to the protein of SEQ ID NO:2n, wherein n is an integer between 1 and 174, can be created by introducing one or more nucleotide substitutions, additions or deletions into the nucleotide sequence of SEQ ID NO:2n−1, wherein n is an integer between 1 and 174, such that one or more amino acid substitutions, additions or deletions are introduced into the encoded protein.

Mutations can be introduced any one of SEQ ID NO:2n−1, wherein n is an integer between 1 and 174, by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the NOVX protein is replaced with another amino acid residue from the same side chain family. Alternatively, in another embodiment, mutations can be introduced randomly along all or part of a NOVX coding sequence, such as by saturation mutagenesis, and the resultant mutants can be screened for NOVX biological activity to identify mutants that retain activity. Following mutagenesis of a nucleic acid of SEQ ID NO:2n−1, wherein n is an integer between 1 and 174, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, HFY, wherein the letters within each group represent the single letter amino acid code.

In one embodiment, a mutant NOVX protein can be assayed for (i) the ability to form protein:protein interactions with other NOVX proteins, other cell-surface proteins, or biologically-active portions thereof, (ii) complex formation between a mutant NOVX protein and a NOVX ligand; or (iii) the ability of a mutant NOVX protein to bind to an intracellular target protein or biologically-active portion thereof; (e.g. avidin proteins).

In yet another embodiment, a mutant NOVX protein can be assayed for the ability to regulate a specific biological function (e.g., regulation of insulin release).

Interfering RNA

In one aspect of the invention, NOVX gene expression can be attenuated by RNA interference. One approach well-known in the art is short interfering RNA (siRNA) mediated gene silencing where expression products of a NOVX gene are targeted by specific double stranded NOVX derived siRNA nucleotide sequences that are complementary to at least a 19-25 nt long segment of the NOVX gene transcript, including the 5′ untranslated (UT) region, the ORF, or the 3′ UT region. See, e.g., PCT applications WO00/44895, WO99/32619, WO01/75164, WO01/92513, WO 01/29058, WO01/89304, WO02/16620, and WO02/29858, each incorporated by reference herein in their entirety. Targeted genes can be a NOVX gene, or an upstream or downstream modulator of the NOVX gene. Nonlimiting examples of upstream or downstream modulators of a NOVX gene include, e.g., a transcription factor that binds the NOVX gene promoter, a kinase or phosphatase that interacts with a NOVX polypeptide, and polypeptides involved in a NOVX regulatory pathway.

According to the methods of the present invention, NOVX gene expression is silenced using short interfering RNA. A NOVX polynucleotide according to the invention includes a siRNA polynucleotide. Such a NOVX siRNA can be obtained using a NOVX polynucleotide sequence, for example, by processing the NOVX ribopolynucleotide sequence in a cell-free system, such as but not limited to a Drosophila extract, or by transcription of recombinant double stranded NOVX RNA or by chemical synthesis of nucleotide sequences homologous to a NOVX sequence. See, e.g., Tuschl, Zamore, Lehmann, Bartel and Sharp (1999), Genes & Dev. 13: 3191-3197, incorporated herein by reference in its entirety. When synthesized, a typical 0.2 micromolar-scale RNA synthesis provides about 1 milligram of siRNA, which is sufficient for 1000 transfection experiments using a 24-well tissue culture plate format.

The most efficient silencing is generally observed with siRNA duplexes composed of a 21-nt sense strand and a 21-nt antisense strand, paired in a manner to have a 2-nt 3′ overhang. The sequence of the 2-nt 3′ overhang makes an additional small contribution to the specificity of siRNA target recognition. The contribution to specificity is localized to the unpaired nucleotide adjacent to the first paired bases. In one embodiment, the nucleotides in the 3′ overhang are ribonucleotides. In an alternative embodiment, the nucleotides in the 3′ overhang are deoxyribonucleotides. Using 2′-deoxyribonucleotides in the 3′ overhangs is as efficient as using ribonucleotides, but deoxyribonucleotides are often cheaper to synthesize and are most likely more nuclease resistant.

A contemplated recombinant expression vector of the invention comprises a NOVX DNA molecule cloned into an expression vector comprising operatively-linked regulatory sequences flanking the NOVX sequence in a manner that allows for expression (by transcription of the DNA molecule) of both strands. An RNA molecule that is antisense to NOVX mRNA is transcribed by a first promoter (e.g., a promoter sequence 3′ of the cloned DNA) and an RNA molecule that is the sense strand for the NOVX mRNA is transcribed by a second promoter (e.g., a promoter sequence 5′ of the cloned DNA). The sense and antisense strands may hybridize in vivo to generate siRNA constructs for silencing of the NOVX gene. Alternatively, two constructs can be utilized to create the sense and anti-sense strands of a siRNA construct. Finally, cloned DNA can encode a construct having secondary structure, wherein a single transcript has both the sense and complementary antisense sequences from the target gene or genes. In an example of this embodiment, a hairpin RNAi product is homologous to all or a portion of the target gene. In another example, a hairpin RNAi product is a siRNA. The regulatory sequences flanking the NOVX sequence may be identical or may be different, such that their expression may be modulated independently, or in a temporal or spatial manner.

In a specific embodiment, siRNAs are transcribed intracellularly by cloning the NOVX gene templates into a vector containing, e.g., a RNA pol III transcription unit from the smaller nuclear RNA (snRNA) U6 or the human RNase P RNA H1. One example of a vector system is the GeneSuppressor™ RNA Interference kit (commercially available from Imgenex). The U6 and H1 promoters are members of the type III class of Pol III promoters. The +1 nucleotide of the U6-like promoters is always guanosine, whereas the +1 for H1 promoters is adenosine. The termination signal for these promoters is defined by five consecutive thymidines. The transcript is typically cleaved after the second uridine. Cleavage at this position generates a 3′ UU overhang in the expressed siRNA, which is similar to the 3′ overhangs of synthetic siRNAs. Any sequence less than 400 nucleotides in length can be transcribed by these promoter, therefore they are ideally suited for the expression of around 21-nucleotide siRNAs in, e.g., an approximately 50-nucleotide RNA stem-loop transcript.

A siRNA vector appears to have an advantage over synthetic siRNAs where long term knock-down of expression is desired. Cells transfected with a siRNA expression vector would experience steady, long-term mRNA inhibition. In contrast, cells transfected with exogenous synthetic siRNAs typically recover from mRNA suppression within seven days or ten rounds of cell division. The long-term gene silencing ability of siRNA expression vectors may provide for applications in gene therapy.

In general, siRNAs are chopped from longer dsRNA by an ATP-dependent ribonuclease called DICER. DICER is a member of the RNase III family of double-stranded RNA-specific endonucleases. The siRNAs assemble with cellular proteins into an endonuclease complex. In vitro studies in Drosophila suggest that the siRNAs/protein complex (siRNP) is then transferred to a second enzyme complex, called an RNA-induced silencing complex (RISC), which contains an endoribonuclease that is distinct from DICER. RISC uses the sequence encoded by the antisense siRNA strand to find and destroy mRNAs of complementary sequence. The siRNA thus acts as a guide, restricting the ribonuclease to cleave only mRNAs complementary to one of the two siRNA strands.

A NOVX mRNA region to be targeted by siRNA is generally selected from a desired NOVX sequence beginning 50 to 100 nt downstream of the start codon. Alternatively, 5′ or 3′ UTRs and regions nearby the start codon can be used but are generally avoided, as these may be richer in regulatory protein binding sites. UTR-binding proteins and/or translation initiation complexes may interfere with binding of the siRNP or RISC endonuclease complex. An initial BLAST homology search for the selected siRNA sequence is done against an available nucleotide sequence library to ensure that only one gene is targeted. Specificity of target recognition by siRNA duplexes indicate that a single point mutation located in the paired region of an siRNA duplex is sufficient to abolish target mRNA degradation. See, Elbashir et al. 2001 EMBO J. 20(23):6877-88. Hence, consideration should be taken to accommodate SNPs, polymorphisms, allelic variants or species-specific variations when targeting a desired gene.

In one embodiment, a complete NOVX siRNA experiment includes the proper negative control. A negative control siRNA generally has the same nucleotide composition as the NOVX siRNA but lack significant sequence homology to the genome. Typically, one would scramble the nucleotide sequence of the NOVX siRNA and do a homology search to make sure it lacks homology to any other gene.

Two independent NOVX siRNA duplexes can be used to knock-down a target NOVX gene. This helps to control for specificity of the silencing effect. In addition, expression of two independent genes can be simultaneously knocked down by using equal concentrations of different NOVX siRNA duplexes, e.g., a NOVX siRNA and an siRNA for a regulator of a NOVX gene or polypeptide. Availability of siRNA-associating proteins is believed to be more limiting than target mRNA accessibility.

A targeted NOVX region is typically a sequence of two adenines (AA) and two thymidines (TT) divided by a spacer region of nineteen (N19) residues (e.g., AA(N19)TT). A desirable spacer region has a G/C-content of approximately 30% to 70%, and more preferably of about 50%. If the sequence AA(N19)TT is not present in the target sequence, an alternative target region would be AA(N21). The sequence of the NOVX sense siRNA corresponds to (N19)TT or N21, respectively. In the latter case, conversion of the 3′ end of the sense siRNA to TT can be performed if such a sequence does not naturally occur in the NOVX polynucleotide. The rationale for this sequence conversion is to generate a symmetric duplex with respect to the sequence composition of the sense and antisense 3′ overhangs. Symmetric 3′ overhangs may help to ensure that the siRNPs are formed with approximately equal ratios of sense and antisense target RNA-cleaving siRNPs. See, e.g., Elbashir, Lendeckel and Tuschl (2001). Genes & Dev. 15: 188-200, incorporated by reference herein in its entirely. The modification of the overhang of the sense sequence of the siRNA duplex is not expected to affect targeted mRNA recognition, as the antisense siRNA strand guides target recognition.

Alternatively, if the NOVX target mRNA does not contain a suitable AA(N21) sequence, one may search for the sequence NA(N21). Further, the sequence of the sense strand and antisense strand may still be synthesized as 5′ (N19)TT, as it is believed that the sequence of the 3′-most nucleotide of the antisense siRNA does not contribute to specificity. Unlike antisense or ribozyme technology, the secondary structure of the target mRNA does not appear to have a strong effect on silencing. See, Harborth, et al. (2001) J. Cell Science 114: 4557-4565, incorporated by reference in its entirety.

Transfection of NOVX siRNA duplexes can be achieved using standard nucleic acid transfection methods, for example, OLIGOFECTAMINE Reagent (commercially available from Invitrogen). An assay for NOVX gene silencing is generally performed approximately 2 days after transfection. No NOVX gene silencing has been observed in the absence of transfection reagent, allowing for a comparative analysis of the wild-type and silenced NOVX phenotypes. In a specific embodiment, for one well of a 24-well plate, approximately 0.84 μg of the siRNA duplex is generally sufficient. Cells are typically seeded the previous day, and are transfected at about 50% confluence. The choice of cell culture media and conditions are routine to those of skill in the art, and will vary with the choice of cell type. The efficiency of transfection may depend on the cell type, but also on the passage number and the confluency of the cells. The time and the manner of formation of siRNA-liposome complexes (e.g. inversion versus vortexing) are also critical. Low transfection efficiencies are the most frequent cause of unsuccessful NOVX silencing. The efficiency of transfection needs to be carefully examined for each new cell line to be used. Preferred cell are derived from a mammal, more preferably from a rodent such as a rat or mouse, and most preferably from a human. Where used for therapeutic treatment, the cells are preferentially autologous, although non-autologous cell sources are also contemplated as within the scope of the present invention.

For a control experiment, transfection of 0.84 μg single-stranded sense NOVX siRNA will have no effect on NOVX silencing, and 0.84 μg antisense siRNA has a weak silencing effect when compared to 0.84 μg of duplex siRNAs. Control experiments again allow for a comparative analysis of the wild-type and silenced NOVX phenotypes. To control for transfection efficiency, targeting of common proteins is typically performed, for example targeting of lamin A/C or transfection of a CMV-driven EGFP-expression plasmid (e.g. commercially available from Clontech). In the above example, a determination of the fraction of lamin A/C knockdown in cells is determined the next day by such techniques as immunofluorescence, Western blot, Northern blot or other similar assays for protein expression or gene expression. Lamin A/C monoclonal antibodies may be obtained from Santa Cruz Biotechnology.

Depending on the abundance and the half life (or turnover) of the targeted NOVX polynucleotide in a cell, a knock-down phenotype may become apparent after 1 to 3 days, or even later. In cases where no NOVX knock-down phenotype is observed, depletion of the NOVX polynucleotide may be observed by immunofluorescence or Western blotting. If the NOVX polynucleotide is still abundant after 3 days, cells need to be split and transferred to a fresh 24-well plate for re-transfection. If no knock-down of the targeted protein is observed, it may be desirable to analyze whether the target mRNA (NOVX or a NOVX upstream or downstream gene) was effectively destroyed by the transfected siRNA duplex. Two days after transfection, total RNA is prepared, reverse transcribed using a target-specific primer, and PCR-amplified with a primer pair covering at least one exon-exon junction in order to control for amplification of pre-mRNAs. RT/PCR of a non-targeted mRNA is also needed as control. Effective depletion of the mRNA yet undetectable reduction of target protein may indicate that a large reservoir of stable NOVX protein may exist in the cell. Multiple transfection in sufficiently long intervals may be necessary until the target protein is finally depleted to a point where a phenotype may become apparent. If multiple transfection steps are required, cells are split 2 to 3 days after transfection. The cells may be transfected immediately after splitting.

An inventive therapeutic method of the invention contemplates administering a NOVX siRNA construct as therapy to compensate for increased or aberrant NOVX expression or activity. The NOVX ribopolynucleotide is obtained and processed into siRNA fragments, or a NOVX siRNA is synthesized, as described above. The NOVX siRNA is administered to cells or tissues using known nucleic acid transfection techniques, as described above. A NOVX siRNA specific for a NOVX gene will decrease or knockdown NOVX transcription products, which will lead to reduced NOVX polypeptide production, resulting in reduced NOVX polypeptide activity in the cells or tissues.

The present invention also encompasses a method of treating a disease or condition associated with the presence of a NOVX protein in an individual comprising administering to the individual an RNAi construct that targets the mRNA of the protein (the mRNA that encodes the protein) for degradation. A specific RNAi construct includes a siRNA or a double stranded gene transcript that is processed into siRNAs. Upon treatment, the target protein is not produced or is not produced to the extent it would be in the absence of the treatment.

Where the NOVX gene function is not correlated with a known phenotype, a control sample of cells or tissues from healthy individuals provides a reference standard for determining NOVX expression levels. Expression levels are detected using the assays described, e.g., RT-PCR, Northern blotting, Western blotting, ELISA, and the like. A subject sample of cells or tissues is taken from a mammal, preferably a human subject, suffering from a disease state. The NOVX ribopolynucleotide is used to produce siRNA constructs, that are specific for the NOVX gene product. These cells or tissues are treated by administering NOVX siRNA's to the cells or tissues by methods described for the transfection of nucleic acids into a cell or tissue, and a change in NOVX polypeptide or polynucleotide expression is observed in the subject sample relative to the control sample, using the assays described. This NOVX gene knockdown approach provides a rapid method for determination of a NOVX minus (NOVX⁻) phenotype in the treated subject sample. The NOVX⁻ phenotype observed in the treated subject sample thus serves as a marker for monitoring the course of a disease state during treatment.

In specific embodiments, a NOVX siRNA is used in therapy. Methods for the generation and use of a NOVX siRNA are known to those skilled in the art. Example techniques are provided below.

Production of RNAs

Sense RNA (ssRNA) and antisense RNA (asRNA) of NOVX are produced using known methods such as transcription in RNA expression vectors. In the initial experiments, the sense and antisense RNA are about 500 bases in length each. The produced ssRNA and asRNA (0.5 μM) in 10 mM Tris-HCl (pH 7.5) with 20 mM NaCl were heated to 95° C. for 1 min then cooled and annealed at room temperature for 12 to 16 h. The RNAs are precipitated and resuspended in lysis buffer (below). To monitor annealing, RNAs are electrophoresed in a 2% agarose gel in TBE buffer and stained with ethidium bromide. See, e.g., Sambrook et al., Molecular Cloning. Cold Spring Harbor Laboratory Press, Plainview, N.Y. (1989).

Lysate Preparation

Untreated rabbit reticulocyte lysate (Ambion) are assembled according to the manufacturer's directions. dsRNA is incubated in the lysate at 30° C. for 10 min prior to the addition of mRNAs. Then NOVX mRNAs are added and the incubation continued for an additional 60 min. The molar ratio of double stranded RNA and mRNA is about 200:1. The NOVX mRNA is radiolabeled (using known techniques) and its stability is monitored by gel electrophoresis.

In a parallel experiment made with the same conditions, the double stranded RNA is internally radiolabeled with a ³²P-ATP. Reactions are stopped by the addition of 2× proteinase K buffer and deproteinized as described previously (Tuschl et al., Genes Dev., 13:3191-3197 (1999)). Products are analyzed by electrophoresis in 15% or 18% polyacrylamide sequencing gels using appropriate RNA standards. By monitoring the gels for radioactivity, the natural production of 10 to 25 nt RNAs from the double stranded RNA can be determined.

The band of double stranded RNA, about 21-23 bps, is eluded. The efficacy of these 21-23 mers for suppressing NOVX transcription is assayed in vitro using the same rabbit reticulocyte assay described above using 50 nanomolar of double stranded 21-23 mer for each assay. The sequence of these 21-23 mers is then determined using standard nucleic acid sequencing techniques.

RNA Preparation

21 nt RNAs, based on the sequence determined above, are chemically synthesized using Expedite RNA phosphoramidites and thymidine phosphoramidite (Proligo, Germany). Synthetic oligonucleotides are deprotected and gel-purified (Elbashir, Lendeckel, & Tuschl, Genes & Dev. 15, 188-200 (2001)), followed by Sep-Pak C18 cartridge (Waters, Milford, Mass., USA) purification (Tuschl, et al., Biochemistry, 32:11658-11668 (1993)).

These RNAs (20 IM) single strands are incubated in annealing buffer (100 mM potassium acetate, 30 mM HEPES-KOH at pH 7.4, 2 mM magnesium acetate) for 1 min at 90° C. followed by 1 h at 37° C.

Cell Culture

A cell culture known in the art to regularly express NOVX is propagated using standard conditions. 24 hours before transfection, at approx. 80% confluency, the cells are trypsinized and diluted 1:5 with fresh medium without antibiotics (1-3×105 cells/ml) and transferred to 24-well plates (500 ml/well). Transfection is performed using a commercially available lipofection kit and NOVX expression is monitored using standard techniques with positive and negative control. A positive control is cells that naturally express NOVX while a negative control is cells that do not express NOVX. Base-paired 21 and 22 nt siRNAs with overhanging 3′ ends mediate efficient sequence-specific mRNA degradation in lysates and in cell culture. Different concentrations of siRNAs are used. An efficient concentration for suppression in vitro in mammalian culture is between 25 nM to 100 nM final concentration. This indicates that siRNAs are effective at concentrations that are several orders of magnitude below the concentrations applied in conventional antisense or ribozyme gene targeting experiments.

The above method provides a way both for the deduction of NOVX siRNA sequence and the use of such siRNA for in vitro suppression. In vivo suppression may be performed using the same siRNA using well known in vivo transfection or gene therapy transfection techniques.

Antisense Nucleic Acids

Another aspect of the invention pertains to isolated antisense nucleic acid molecules that are hybridizable to or complementary to the nucleic acid molecule comprising the nucleotide sequence of SEQ ID NO:2n−1, wherein n is an integer between 1 and 174, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of a NOVX protein of SEQ ID NO:2n, wherein n is an integer between 1 and 174, or antisense nucleic acids complementary to a NOVX nucleic acid sequence of SEQ ID NO:2n−1, wherein n is an integer between 1 and 174, are additionally provided.

In one embodiment, an antisense nucleic acid molecule is antisense to a “coding region” of the coding strand of a nucleotide sequence encoding a NOVX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the NOVX protein. The term “noncoding region” refers to 5′ and 3′ sequences which flank the coding region that are not translated into amino acids (i.e., also referred to as 5′ and 3′ untranslated regions).

Given the coding strand sequences encoding the NOVX protein disclosed herein, antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-carboxymethylaminomethyl-2-thiouridine, 5-(carboxyhydroxylmethyl) uracil, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 5-methoxyuracil, 3-methylcytosine, 5-methylcytosine, N6-adenine, 7-methylguanine, 5-methylaminomethyluracil, 5-methoxyaminomethyl-2-thiouracil, 2-thiouracil, 4-thiouracil, beta-D-mannosylqueosine, 5′-methoxycarboxymethyluracil, 2-methylthio-N6-isopentenyladenine, uracil-5-oxyacetic acid (v), wybutoxosine, pseudouracil, queosine, 2-thiocytosine, 5-methyl-2-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a NOVX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An (x-anomeric nucleic acid molecule forms specific double-stranded hybrids with complementary RNA in which, contrary to the usual β-units, the strands run parallel to each other. See, e.g., Gaultier, et al., 1987. Nucl. Acids Res. 15: 6625-6641. The antisense nucleic acid molecule can also comprise a 2′-o-methylribonucleotide (See, e.g., Inoue, et al. 1987. Nucl. Acids Res. 15: 6131-6148) or a chimeric RNA-DNA analogue (See, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave NOVX mRNA transcripts to thereby inhibit translation of NOVX mRNA. A ribozyme having specificity for a NOVX-encoding nucleic acid can be designed based upon the nucleotide sequence of a NOVX cDNA disclosed herein (i.e., SEQ ID NO:2n−1, wherein n is an integer between 1 and 174). For example, a derivative of a Tetrahymena L-19 IVS RNA can be constructed in which the nucleotide sequence of the active site is complementary to the nucleotide sequence to be cleaved in a NOVX-encoding mRNA. See, e.g., U.S. Pat. No. 4,987,071 to Cech, et al. and U.S. Pat. No. 5,116,742 to Cech, et al. NOVX mRNA can also be used to select a catalytic RNA having a specific ribonuclease activity from a pool of RNA molecules. See, e.g., Bartel et al., (1993) Science 261:1411-1418.

Alternatively, NOVX gene expression can be inhibited by targeting nucleotide sequences complementary to the regulatory region of the NOVX nucleic acid (e.g., the NOVX promoter and/or enhancers) to form triple helical structures that prevent transcription of the NOVX gene in target cells. See, e.g., Helene, 1991. Anticancer Drug Des. 6: 569-84; Helene, et al. 1992. Ann. N.Y. Acad. Sci. 660: 27-36; Maher, 1992. Bioassays 14: 807-15.

In various embodiments, the NOVX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleotide bases are retained. The neutral backbone of PNAs has been shown to allow for specific hybridization to DNA and RNA under conditions of low ionic strength. The synthesis of PNA oligomer can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, for example, in the analysis of single base pair mutations in a gene (e.g., PNA directed PCR clamping; as artificial restriction enzymes when used in combination with other enzymes, e.g., S₁ nucleases (See, Hyrup, et al., 1996.supra); or as probes or primers for DNA sequence and hybridization (See, Hyrup, et al., 1996, supra; Perry-O'Keefe, et al., 1996. supra).

In another embodiment, PNAs of NOVX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOVX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleotide bases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g. 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

NOVX Polypeptides

A polypeptide according to the invention includes a polypeptide including the amino acid sequence of NOVX polypeptides whose sequences are provided in any one of SEQ ID NO:2n, wherein n is an integer between 1 and 174. The invention also includes a mutant or variant protein any of whose residues may be changed from the corresponding residues shown in any one of SEQ ID NO:2n, wherein n is an integer between 1 and 174, while still encoding a protein that maintains its NOVX activities and physiological functions, or a functional fragment thereof.

In general, a NOVX variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOVX proteins, and biologically-active portions thereof, or derivatives, fragments, analogs or homologs thereof. Also provided are polypeptide fragments suitable for use as immunogens to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, a NOVX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX proteins in which the protein is separated from cellular components of the cells from which it is isolated or recombinantly-produced. In one embodiment, the language “substantially free of cellular material” includes preparations of NOVX proteins having less than about 30% (by dry weight) of non-NOVX proteins (also referred to herein as a “contaminating protein”), more preferably less than about 20% of non-NOVX proteins, still more preferably less than about 10% of non-NOVX proteins, and most preferably less than about 5% of non-NOVX proteins. When the NOVX protein or biologically-active portion thereof is recombinantly-produced, it is also preferably substantially free of culture medium, i.e., culture medium represents less than about 20%, more preferably less than about 10%, and most preferably less than about 5% of the volume of the NOVX protein preparation.

The language “substantially free of chemical precursors or other chemicals” includes preparations of NOVX proteins in which the protein is separated from chemical precursors or other chemicals that are involved in the synthesis of the protein. In one embodiment, the language “substantially free of chemical precursors or other chemicals” includes preparations of NOVX proteins having less than about 30% (by dry weight) of chemical precursors or non-NOVX chemicals, more preferably less than about 20% chemical precursors or non-NOVX chemicals, still more preferably less than about 10% chemical precursors or non-NOVX chemicals, and most preferably less than about 5% chemical precursors or non-NOVX chemicals.

Biologically-active portions of NOVX proteins include peptides comprising amino acid sequences sufficiently homologous to or derived from the amino acid sequences of the NOVX proteins (e.g., the amino acid sequence of SEQ ID NO:2n, wherein n is an integer between 1 and 174) that include fewer amino acids than the full-length NOVX proteins, and exhibit at least one activity of a NOVX protein. Typically, biologically-active portions comprise a domain or motif with at least one activity of the NOVX protein. A biologically-active portion of a NOVX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native NOVX protein.

In an embodiment, the NOVX protein has an amino acid sequence of SEQ ID NO:2n, wherein n is an integer between 1 and 174. In other embodiments, the NOVX protein is substantially homologous to SEQ ID NO:2n, wherein n is an integer between 1 and 174, and retains the functional activity of the protein of SEQ ID NO:2n, wherein n is an integer between 1 and 174, yet differs in amino acid sequence due to natural allelic variation or mutagenesis, as described in detail, below. Accordingly, in another embodiment, the NOVX protein is a protein that comprises an amino acid sequence at least about 45% homologous to the amino acid sequence of SEQ ID NO:2n, wherein n is an integer between 1 and 174, and retains the functional activity of the NOVX proteins of SEQ ID NO:2n, wherein n is an integer between 1 and 174.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence of SEQ ID NO:2n−1, wherein n is an integer between 1 and 174.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins,

The invention also provides NOVX chimeric or fusion proteins. As used herein, a NOVX “chimeric protein” or “fusion protein” comprises a NOVX polypeptide operatively-linked to a non-NOVX polypeptide. An “NOVX polypeptide” refers to a polypeptide having an amino acid sequence corresponding to a NOVX protein of SEQ ID NO:2n, wherein n is an integer between 1 and 174, whereas a “non-NOVX polypeptide” refers to a polypeptide having an amino acid sequence corresponding to a protein that is not substantially homologous to the NOVX protein, e.g., a protein that is different from the NOVX protein and that is derived from the same or a different organism. Within a NOVX fusion protein the NOVX polypeptide can correspond to all or a portion of a NOVX protein. In one embodiment, a NOVX fusion protein comprises at least one biologically-active portion of a NOVX protein. In another embodiment, a NOVX fusion protein comprises at least two biologically-active portions of a NOVX protein. In yet another embodiment, a NOVX fusion protein comprises at least three biologically-active portions of a NOVX protein. Within the fusion protein, the term “operatively-linked” is intended to indicate that the NOVX polypeptide and the non-NOVX polypeptide are fused in-frame with one another. The non-NOVX polypeptide can be fused to the N-terminus or C-terminus of the NOVX polypeptide.

In one embodiment, the fusion protein is a GST-NOVX fusion protein in which the NOVX sequences are fused to the C-terminus of the GST (glutathione S-transferase) sequences. Such fusion proteins can facilitate the purification of recombinant NOVX polypeptides.

In another embodiment, the fusion protein is a NOVX protein containing a heterologous signal sequence at its N-terminus. In certain host cells (e.g., mammalian host cells), expression and/or secretion of NOVX can be increased through use of a heterologous signal sequence.

In yet another embodiment, the fusion protein is a NOVX-immunoglobulin fusion protein in which the NOVX sequences are fused to sequences derived from a member of the immunoglobulin protein family. The NOVX-immunoglobulin fusion proteins of the invention can be incorporated into pharmaceutical compositions and administered to a subject to inhibit an interaction between a NOVX ligand and a NOVX protein on the surface of a cell, to thereby suppress NOVX-mediated signal transduction in vivo. The NOVX-immunoglobulin fusion proteins can be used to affect the bioavailability of a NOVX cognate ligand. Inhibition of the NOVX ligand/NOVX interaction may be useful therapeutically for both the treatment of proliferative and differentiative disorders, as well as modulating (e.g. promoting or inhibiting) cell survival. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with a NOVX ligand.

A NOVX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). A NOVX-encoding nucleic acid can be cloned into such an expression vector such that the fusion moiety is linked in-frame to the NOVX protein.

NOVX Agonists and Antagonists

The invention also pertains to variants of the NOVX proteins that function as either NOVX agonists (i.e., mimetics) or as NOVX antagonists. Variants of the NOVX protein can be generated by mutagenesis (e.g., discrete point mutation or truncation of the NOVX protein). An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX proteins.

Variants of the NOVX proteins that function as either NOVX agonists (i.e., mimetics) or as NOVX antagonists can be identified by screening combinatorial libraries of mutants (e.g., truncation mutants) of the NOVX proteins for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins (e.g., for phage display) containing the set of NOVX sequences therein. There are a variety of methods which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes-allows for the provision, in one mixture, of all of the sequences encoding the desired set of potential NOVX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the NOVX protein coding sequences can be used to generate a variegated population of NOVX fragments for screening and subsequent selection of variants of a NOVX protein. In one embodiment, a library of coding sequence fragments can be generated by treating a double stranded PCR fragment of a NOVX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S₁ nuclease, and ligating the resulting fragment library into an expression vector. By this method, expression libraries can be derived which encodes N-terminal and internal fragments of various sizes of the NOVX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOVX variants. See, e.g., Arkin and Yourvan, 1992. Proc. Natl. Acad. Sci. USA 89: 7811-7815; Delgrave, et al., 1993. Protein Engineering 6:327-331.

Anti-NOVX Antibodies

Included in the invention are antibodies to NOVX proteins, or fragments of NOVX proteins. The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab}, and F_{(ab′)2} fragments, and an F_{ab} expression library. In general, antibody molecules obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated protein of the invention intended to serve as an antigen, or a portion or fragment thereof, can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein, such as an amino acid sequence of SEQ ID NO:2n, wherein n is an integer between 1 and 174, and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of NOVX that is located on the surface of the protein, e.g. a hydrophilic region. A hydrophobicity analysis of the human NOVX protein sequence will indicate which regions of a NOVX polypeptide are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each incorporated herein by reference in their entirety. Antibodies that are specific for one or more domains within an antigenic protein, or derivatives, fragments, analogs or homologs thereof, are also provided herein.

The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. A NOVX polypeptide or a fragment thereof comprises at least one antigenic epitope. An anti-NOVX antibody of the present invention is said to specifically bind to antigen NOVX when the equilibrium binding constant (K_{D}) is ≦1 μM, preferably ≦100 nM, more preferably ≦10 nM, and most preferably ≦100 pM to about 1 pM, as measured by assays including radioligand binding assays or similar assays known to skilled artisans.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human mycloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). It is an objective, especially important in therapeutic applications of monoclonal antibodies, to identify antibodies having a high degree of specificity and a high binding affinity for the target antigen.

After the desired hybridoma cells are identified, the clones can be subcloned by limiting dilution procedures and grown by standard methods (Goding, 1986). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or mycloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fe), typically that of a human immunoglobulin (Jones et al., 1986; Riechmann et al., 1988; and Presta, Curr. Op. Struct. Biol., 2:593-596 (1992)).

Human Antibodies

Fully human antibodies essentially relate to antibody molecules in which the entire sequence of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al, (Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of Fab expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab)2} fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobuiin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), dilsocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

Immunoliposomes

The antibodies disclosed herein can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem., 257: 286-288 (1982) via a disulfide-interchange reaction. A chemotherapeutic agent (such as Doxorubicin) is optionally contained within the liposome. See Gabizon et al., J. National Cancer Inst., 81(19): 1484 (1989).

Diagnostic Applications of Antibodies Directed Against the Proteins of the Invention

In one embodiment, methods for the screening of antibodies that possess the desired specificity include, but are not limited to, enzyme linked immunosorbent assay (ELISA) and other immunologically mediated techniques known within the art. In a specific embodiment, selection of antibodies that are specific to a particular domain of an NOVX protein is facilitated by generation of hybridomas that bind to the fragment of an NOVX protein possessing such a domain. Thus, antibodies that are specific for a desired domain within an NOVX protein, or derivatives, fragments, analogs or homologs thereof, are also provided herein.

Antibodies directed against a NOVX protein of the invention may be used in methods known within the art relating to the localization and/or quantitation of a NOVX protein (e.g., for use in measuring levels of the NOVX protein within appropriate physiological samples, for use in diagnostic methods, for use in imaging the protein, and the like). In a given embodiment, antibodies specific to a NOVX protein, or derivative, fragment, analog or homolog thereof, that contain the antibody derived antigen binding domain, are utilized as pharmacologically active compounds (referred to hereinafter as “Therapeutics”).

An antibody specific for a NOVX protein of the invention (e.g., a monoclonal antibody or a polyclonal antibody) can be used to isolate a NOVX polypeptide by standard techniques, such as immunoaffinity, chromatography or immunoprecipitation. An antibody to a NOVX polypeptide can facilitate the purification of a natural NOVX antigen from cells, or of a recombinantly produced NOVX antigen expressed in host cells. Moreover, such an anti-NOVX antibody can be used to detect the antigenic NOVX protein (e.g., in a cellular lysate or cell supernatant) in order to evaluate the abundance and pattern of expression of the antigenic NOVX protein. Antibodies directed against a NOVX protein can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Antibody Therapeutics

Antibodies of the invention, including polyclonal, monoclonal, humanized and fully human antibodies, may used as therapeutic agents. Such agents will generally be employed to treat or prevent a disease or pathology in a subject. An antibody preparation, preferably one having high specificity and high affinity for its target antigen, is administered to the subject and will generally have an effect due to its binding with the target. Such an effect may be one of two kinds, depending on the specific nature of the interaction between the given antibody molecule and the target antigen in question. In the first instance, administration of the antibody may abrogate or inhibit the binding of the target with an endogenous ligand to which it naturally binds. In this case, the antibody binds to the target and masks a binding site of the naturally occurring ligand, wherein the ligand serves as an effector molecule. Thus the receptor mediates a signal transduction pathway for which ligand is responsible.

Alternatively, the effect may be one in which the antibody elicits a physiological result by virtue of binding to an effector binding site on the target molecule. In this case the target, a receptor having an endogenous ligand which may be absent or defective in the disease or pathology, binds the antibody as a surrogate effector ligand, initiating a receptor-based signal transduction event by the receptor.

A therapeutically effective amount of an antibody of the invention relates generally to the amount needed to achieve a therapeutic objective. As noted above, this may be a binding interaction between the antibody and its target antigen that, in certain cases, interferes with the functioning of the target, and in other cases, promotes a physiological response. The amount required to be administered will furthermore depend on the binding affinity of the antibody for its specific antigen, and will also depend on the rate at which an administered antibody is depleted from the free volume other subject to which it is administered. Common ranges for therapeutically effective dosing of an antibody or antibody fragment of the invention may be, by way of nonlimiting example, from about 0.1 mg/kg body weight to about 50 mg/kg body weight. Common dosing frequencies may range, for example, from twice daily to once a week.

Pharmaceutical Compositions of Antibodies

Antibodies specifically binding a protein of the invention, as well as other molecules identified by the screening assays disclosed herein, can be administered for the treatment of various disorders in the form of pharmaceutical compositions. Principles and considerations involved in preparing such compositions, as well as guidance in the choice of components are provided, for example, in Remington: The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

If the antigenic protein is intracellular and whole antibodies are used as inhibitors, internalizing antibodies are preferred. However, liposomes can also be used to deliver the antibody, or an antibody fragment, into cells. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al., Proc. Natl. Acad. Sci. USA, 90: 7889-7893 (1993). The formulation herein can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

ELISA Assay

An agent for detecting an analyte protein is an antibody capable of binding to an analyte protein, preferably an antibody with a detectable label. Antibodies can be polyclonal, or more preferably, monoclonal. An intact antibody, or a fragment thereof (e.g., F_{ab} or F_{(ab)2}) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of the invention can be used to detect an analyte mRNA, protein, or genomic DNA in a biological sample in vitro as well as in vivo. For example, in vitro techniques for detection of an analyte mRNA include Northern hybridizations and in situ hybridizations. In vitro techniques for detection of an analyte protein include enzyme linked immunosorbent assays (ELISAs), Western blots, immunoprecipitations, and immunofluorescence. In vitro techniques for detection of an analyte genomic DNA include Southern hybridizations. Procedures for conducting immunoassays are described, for example in “ELISA: Theory and Practice: Methods in Molecular Biology”, Vol. 42, J. R. Crowther (Ed.) Human Press, Totowa, N.J., 1995; “Immunoassay”, E. Diamandis and T. Christopoulus, Academic Press, Inc., San Diego, Calif., 1996; and “Practice and Theory of Enzyme Immunoassays”, P. Tijssen, Elsevier Science Publishers, Amsterdam, 1985. Furthermore, in vivo techniques for detection of an analyte protein include introducing into a subject a labeled anti-an analyte protein antibody. For example, the antibody can be labeled with a radioactive marker whose presence and location in a subject can be detected by standard imaging techniques.

NOVX Recombinant Expression Vectors and Host Cells

Another aspect of the invention pertains to vectors, preferably expression vectors, containing a nucleic acid encoding a NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NOVX proteins, mutant forms of NOVX proteins, fusion proteins, etc.).

The recombinant expression vectors of the invention can be designed for expression of NOVX proteins in prokaryotic or eukaryotic cells. For example, NOVX proteins can be expressed in bacterial cells such as Escherichia coli, insect cells (using baculovirus expression vectors) yeast cells or mammalian cells. Suitable host cells arc discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the NOVX expression vector is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, NOVX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOVX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

Transgenic NOVX Animals

The host cells of the invention can also be used to produce non-human transgenic animals. For example, in one embodiment, a host cell of the invention is a fertilized oocyte or an embryonic stem cell into which NOVX protein-coding sequences have been introduced. Such host cells can then be used to create non-human transgenic animals in which exogenous NOVX sequences have been introduced into their genome or homologous recombinant animals in which endogenous NOVX sequences have been altered. Such animals are useful for studying the function and/or activity of NOVX protein and for identifying and/or evaluating modulators of NOVX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOVX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing NOVX-encoding nucleic acid into the male pronuclei of a fertilized oocyte (e.g., by microinjection, retroviral infection) and allowing the oocyte to develop in a pseudopregnant female foster animal. The human NOVX cDNA sequences, i.e., any one of SEQ ID NO:2n−1, wherein n is an integer between 1 and 174, can be introduced as a transgene into the genome of a non-human animal. Alternatively, a non-human homologue of the human NOVX gene, such as a mouse NOVX gene, can be isolated based on hybridization to the human NOVX cDNA (described further supra) and used as a transgene. Intronic sequences and polyadenylation signals can also be included in the transgene to increase the efficiency of expression of the transgene. A tissue-specific regulatory sequence(s) can be operably-linked to the NOVX transgene to direct expression of NOVX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the NOVX transgene in its genome and/or expression of NOVX mRNA in tissues or cells of the animals. A transgenic founder animal can then be used to breed additional animals carrying the transgene. Moreover, transgenic animals carrying a transgene-encoding NOVX protein can further be bred to other transgenic animals carrying other transgenes.

To create a homologous recombinant animal, a vector is prepared which contains at least a portion of a NOVX gene into which a deletion, addition or substitution has been introduced to thereby alter, e.g., functionally disrupt, the NOVX gene. The NOVX gene can be a human gene (e.g., the cDNA of any one of SEQ ID NO:2n−1, wherein n is an integer between 1 and 174), but more preferably, is a non-human homologue of a human NOVX gene. For example, a mouse homologue of human NOVX gene of SEQ ID NO:2n−1, wherein n is an integer between 1 and 174, can be used to construct a homologous recombination vector suitable for altering an endogenous NOVX gene in the mouse genome. In one embodiment, the vector is designed such that, upon homologous recombination, the endogenous NOVX gene is functionally disrupted (i.e., no longer encodes a functional protein; also referred to as a “knock out” vector).

Alternatively, the vector can be designed such that, upon homologous recombination, the endogenous NOVX gene is mutated or otherwise altered but still encodes functional protein (e.g., the upstream regulatory region can be altered to thereby alter the expression of the endogenous NOVX protein). In the homologous recombination vector, the altered portion of the NOVX gene is flanked at its 5′- and 3′-termini by additional nucleic acid of the NOVX gene to allow for homologous recombination to occur between the exogenous NOVX gene carried by the vector and an endogenous NOVX gene in an embryonic stem cell. The additional flanking NOVX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOVX gene has homologously-recombined with the endogenous NOVX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express NOVX protein (e.g., via a recombinant expression vector in a host cell in gene therapy applications), to detect NOVX mRNA (e.g., in a biological sample) or a genetic lesion in a NOVX gene, and to modulate NOVX activity, as described further, below. In addition, the NOVX proteins can be used to screen drugs or compounds that modulate the NOVX protein activity or expression as well as to treat disorders characterized by insufficient or excessive production of NOVX protein or production of NOVX protein forms that have decreased or aberrant activity compared to NOVX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease (possesses anti-microbial activity) and the various dyslipidemias. In addition, the anti-NOVX antibodies of the invention can be used to detect and isolate NOVX proteins and modulate NOVX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to NOVX proteins or have a stimulatory or inhibitory effect on, e.g., NOVX protein expression or NOVX protein activity. The invention also includes compounds identified in the screening assays described herein.

In one embodiment, the invention provides assays for screening candidate or test compounds which bind to or modulate the activity of the membrane-bound form of a NOVX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37:1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of NOVX protein, or a biologically-active portion thereof, on the cell surface is contacted with a test compound and the ability of the test compound to bind to a NOVX protein determined. The cell, for example, can of mammalian origin or a yeast cell. Determining the ability of the test compound to bind to the NOVX protein can be accomplished, for example, by coupling the test compound with a radioisotope or enzymatic label such that binding of the test compound to the NOVX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOVX protein, or a biologically-active portion thereof, on the cell surface with a known compound which binds NOVX to form an assay mixture, contacting the assay mixture with a test compound, and determining the ability of the test compound to interact with a NOVX protein, wherein determining the ability of the test compound to interact with a NOVX protein comprises determining the ability of the test compound to preferentially bind to NOVX protein or a biologically-active portion thereof as compared to the known compound.

In another embodiment, an assay is a cell-based assay comprising contacting a cell expressing a membrane-bound form of NOVX protein, or a biologically-active portion thereof, on the cell surface with a test compound and determining the ability of the test compound to modulate (e.g., stimulate or inhibit) the activity of the NOVX protein or biologically-active portion thereof. Determining the ability of the test compound to modulate the activity of NOVX or a biologically-active portion thereof can be accomplished, for example, by determining the ability of the NOVX protein to bind to or interact with a NOVX target molecule. As used herein, a “target molecule” is a molecule with which a NOVX protein binds or interacts in nature, for example, a molecule on the surface of a cell which expresses a NOVX interacting protein, a molecule on the surface of a second cell, a molecule in the extracellular milieu, a molecule associated with the internal surface of a cell membrane or a cytoplasmic molecule. A NOVX target molecule can be a non-NOVX molecule or a NOVX protein or polypeptide of the invention. In one embodiment, a NOVX target molecule is a component of a signal transduction pathway that facilitates transduction of an extracellular signal (e.g. a signal generated by binding of a compound to a membrane-bound NOVX molecule) through the cell membrane and into the cell. The target, for example, can be a second intercellular protein that has catalytic activity or a protein that facilitates the association of downstream signaling molecules with NOVX.

Determining the ability of the NOVX protein to bind to or interact with a NOVX target molecule can be accomplished by one of the methods described above for determining direct binding. In one embodiment, determining the ability of the NOVX protein to bind to or interact with a NOVX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising a NOVX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting a NOVX protein or biologically-active portion thereof with a test compound and determining the ability of the test compound to bind to the NOVX protein or biologically-active portion thereof. Binding of the test compound to the NOVX protein can be determined either directly or indirectly as described above. In one such embodiment, the assay comprises contacting the NOVX protein or biologically-active portion thereof with a known compound which binds NOVX to form an assay mixture, contacting the assay mixture with a test compound, and determining the ability of the test compound to interact with a NOVX protein, wherein determining the ability of the test compound to interact with a NOVX protein comprises determining the ability of the test compound to preferentially bind to NOVX or biologically-active portion thereof as compared to the known compound.

In still another embodiment, an assay is a cell-free assay comprising contacting NOVX protein or biologically-active portion thereof with a test compound and determining the ability of the test compound to modulate (e.g. stimulate or inhibit) the activity of the NOVX protein or biologically-active portion thereof. Determining the ability of the test compound to modulate the activity of NOVX can be accomplished, for example, by determining the ability of the NOVX protein to bind to a NOVX target molecule by one of the methods described above for determining direct binding. In an alternative embodiment, determining the ability of the test compound to modulate the activity of NOVX protein can be accomplished by determining the ability of the NOVX protein further modulate a NOVX target molecule. For example, the catalytic/enzymatic activity of the target molecule on an appropriate substrate can be determined as described, supra.

In yet another embodiment, the cell-free assay comprises contacting the NOVX protein or biologically-active portion thereof with a known compound which binds NOVX protein to form an assay mixture, contacting the assay mixture with a test compound, and determining the ability of the test compound to interact with a NOVX protein, wherein determining the ability of the test compound to interact with a NOVX protein comprises determining the ability of the NOVX protein to preferentially bind to or modulate the activity of a NOVX target molecule.

The cell-free assays of the invention are amenable to use of both the soluble form or the membrane-bound form of NOVX protein. In the case of cell-free assays comprising the membrane-bound form of NOVX protein, it may be desirable to utilize a solubilizing agent such that the membrane-bound form of NOVX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl-N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either NOVX protein or its target molecule to facilitate separation of complexed from uncomplexed forms of one or both of the proteins, as well as to accommodate automation of the assay. Binding of a test compound to NOVX protein, or interaction of NOVX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOVX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOVX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOVX protein binding or activity determined using standard techniques.

Other techniques for immobilizing proteins on matrices can also be used in the screening assays of the invention. For example, either the NOVX protein or its target molecule can be immobilized utilizing conjugation of biotin and streptavidin. Biotinylated NOVX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with NOVX protein or target molecules, but which do not interfere with binding of the NOVX protein to its target molecule, can be derivatized to the wells of the plate, and unbound target or NOVX protein trapped in the wells by antibody conjugation. Methods for detecting such complexes, in addition to those described above for the GST-immobilized complexes, include immunodetection of complexes using antibodies reactive with the NOVX protein or target molecule, as well as enzyme-linked assays that rely on detecting an enzymatic activity associated with the NOVX protein or target molecule.

In another embodiment, modulators of NOVX protein expression are identified in a method wherein a cell is contacted with a candidate compound and the expression of NOVX mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX mRNA or protein is greater (i.e., statistically significantly greater) in the presence of the candidate compound than in its absence, the candidate compound is identified as a stimulator of NOVX mRNA or protein expression. Alternatively, when expression of NOVX mRNA or protein is less (statistically significantly less) in the presence of the candidate compound than in its absence, the candidate compound is identified as an inhibitor of NOVX mRNA or protein expression. The level of NOVX mRNA or protein expression in the cells can be determined by methods described herein for detecting NOVX mRNA or protein.

In yet another aspect of the invention, the NOVX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with NOVX (“NOVX-binding proteins” or “NOVX-bp”) and modulate NOVX activity. Such NOVX-binding proteins are also involved in the propagation of signals by the NOVX proteins as, for example, upstream or downstream elements of the NOVX pathway.

The two-hybrid system is based on the modular nature of most transcription factors, which consist of separable DNA-binding and activation domains. Briefly, the assay utilizes two different DNA constructs. In one construct, the gene that codes for NOVX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a NOVX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOVX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the NOVX sequences of SEQ ID NO:2n−1, wherein n is an integer between 1 and 174, or fragments or derivatives thereof, can be used to map the location of the NOVX genes, respectively, on a chromosome. The mapping of the NOVX sequences to chromosomes is an important first step in correlating these sequences with genes associated with disease.

Briefly, NOVX genes can be mapped to chromosomes by preparing PCR primers (preferably 15-25 bp in length) from the NOVX sequences. Computer analysis of the NOVX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOVX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NOVX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g. in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the NOVX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The NOVX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the NOVX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOVX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If coding sequences, such as those of SEQ ID NO:2n−1, wherein n is an integer between 1 and 174, are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX activity, in the context of a biological sample (e.g., blood, serum, cells, tissue) to thereby determine whether an individual is afflicted with a disease or disorder, or is at risk of developing a disorder, associated with aberrant NOVX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with NOVX protein, nucleic acid expression or activity. For example, mutations in a NOVX gene can be assayed in a biological sample. Such assays can be used for prognostic or predictive purpose to thereby prophylactically treat an individual prior to the onset of a disorder characterized by or associated with NOVX protein, nucleic acid expression, or biological activity.

Another aspect of the invention provides methods for determining NOVX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.) Yet another aspect of the invention pertains to monitoring the influence of agents (e.g., drugs, compounds) on the expression or activity of NOVX in clinical trials.

These and other agents are described in further detail in the following sections.

Diagnostic Assays

An exemplary method for detecting the presence or absence of NOVX in a biological sample involves obtaining a biological sample from a test subject and contacting the biological sample with a compound or an agent capable of detecting NOVX protein or nucleic acid (e.g., mRNA, genomic DNA) that encodes NOVX protein such that the presence of NOVX is detected in the biological sample. An agent for detecting NOVX mRNA or genomic DNA is a labeled nucleic acid probe capable of hybridizing to NOVX mRNA or genomic DNA. The nucleic acid probe can be, for example, a full-length NOVX nucleic acid, such as the nucleic acid of SEQ ID NO:2n−1, wherein n is an integer between 1 and 174, or a portion thereof, such as an oligonucleotide of at least 15, 30, 50, 100, 250 or 500 nucleotides in length and sufficient to specifically hybridize under stringent conditions to NOVX mRNA or genomic DNA. Other suitable probes for use in the diagnostic assays of the invention are described herein.

An agent for detecting NOVX protein is an antibody capable of binding to NOVX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOVX mRNA, protein, or genomic DNA in a biological sample in vitro as well as in vivo. For example, in vitro techniques for detection of NOVX mRNA include Northern hybridizations and in situ hybridizations. In vitro techniques for detection of NOVX protein include enzyme linked immunosorbent assays (ELISAs), Western blots, immunoprecipitations, and immunofluorescence. In vitro techniques for detection of NOVX genomic DNA include Southern hybridizations. Furthermore, in vivo techniques for detection of NOVX protein include introducing into a subject a labeled anti-NOVX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting NOVX protein, mRNA, or genomic DNA, such that the presence of NOVX protein, mRNA or genomic DNA is detected in the biological sample, and comparing the presence of NOVX protein, mRNA or genomic DNA in the control sample with the presence of NOVX protein, mRNA or genomic DNA in the test sample.

The invention also encompasses kits for detecting the presence of NOVX in a biological sample. For example, the kit can comprise: a labeled compound or agent capable of detecting NOVX protein or mRNA in a biological sample; means for determining the amount of NOVX in the sample; and means for comparing the amount of NOVX in the sample with a standard. The compound or agent can be packaged in a suitable container. The kit can further comprise instructions for using the kit to detect NOVX protein or nucleic acid.

Prognostic Assays

The diagnostic methods described herein can furthermore be utilized to identify subjects having or at risk of developing a disease or disorder associated with aberrant NOVX expression or activity. For example, the assays described herein, such as the preceding diagnostic assays or the following assays, can be utilized to identify a subject having or at risk of developing a disorder associated with NOVX protein, nucleic acid expression or activity. Alternatively, the prognostic assays can be utilized to identify a subject having or at risk for developing a disease or disorder. Thus, the invention provides a method for identifying a disease or disorder associated with aberrant NOVX expression or activity in which a test sample is obtained from a subject and NOVX protein or nucleic acid (e.g., mRNA, genomic DNA) is detected, wherein the presence of NOVX protein or nucleic acid is diagnostic for a subject having or at risk of developing a disease or disorder associated with aberrant NOVX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOVX expression or activity. For example, such methods can be used to determine whether a subject can be effectively treated with an agent for a disorder. Thus, the invention provides methods for determining whether a subject can be effectively treated with an agent for a disorder associated with aberrant NOVX expression or activity in which a test sample is obtained and NOVX protein or nucleic acid is detected (e.g., wherein the presence of NOVX protein or nucleic acid is diagnostic for a subject that can be administered the agent to treat a disorder associated with aberrant NOVX expression or activity).

The methods of the invention can also be used to detect genetic lesions in a NOVX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding a NOVX-protein, or the misexpression of the NOVX gene. For example, such genetic lesions can be detected by ascertaining the existence of at least one of: (i) a deletion of one or more nucleotides from a NOVX gene; (ii) an addition of one or more nucleotides to a NOVX gene; (iii) a substitution of one or more nucleotides of a NOVX gene, (iv) a chromosomal rearrangement of a NOVX gene; (v) an alteration in the level of a messenger RNA transcript of a NOVX gene, (vi) aberrant modification of a NOVX gene, such as of the methylation pattern of the genomic DNA, (vii) the presence of a non-wild-type splicing pattern of a messenger RNA transcript of a NOVX gene, (viii) a non-wild-type level of a NOVX protein, (ix) allelic loss of a NOVX gene, and (x) inappropriate post-translational modification of a NOVX protein. As described herein, there are a large number of assay techniques known in the art which can be used for detecting lesions in a NOVX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOVX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to a NOVX gene under conditions such that hybridization and amplification of the NOVX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a NOVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOVX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in NOVX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOVX gene and detect mutations by comparing the sequence of the sample NOVX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the NOVX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type NOVX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on a NOVX sequence, e.g., a wild-type NOVX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in NOVX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control NOVX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a NOVX gene.

Furthermore, any cell type or tissue, preferably peripheral blood leukocytes, in which NOVX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on NOVX activity (e.g., NOVX gene expression), as identified by a screening assay described herein can be administered to individuals to treat (prophylactically or therapeutically) disorders. The disorders include but are not limited to, e.g., those diseases, disorders and conditions listed above, and more particularly include those diseases, disorders, or conditions associated with homologs of a NOVX protein, such as those summarized in Table A.

In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOVX protein, expression of NOVX nucleic acid, or mutation content of NOVX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome pregnancy zone protein precursor enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of NOVX protein, expression of NOVX nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a NOVX modulator, such as a modulator identified by one of the exemplary screening assays described herein.

Monitoring of Effects During Clinical Trials

Monitoring the influence of agents (e.g., drugs, compounds) on the expression or activity of NOVX (e.g., the ability to modulate aberrant cell proliferation and/or differentiation) can be applied not only in basic drug screening, but also in clinical trials. For example, the effectiveness of an agent determined by a screening assay as described herein to increase NOVX gene expression, protein levels, or upregulate NOVX activity, can be monitored in clinical trails of subjects exhibiting decreased NOVX gene expression, protein levels, or downregulated NOVX activity. Alternatively, the effectiveness of an agent determined by a screening assay to decrease NOVX gene expression, protein levels, or downregulate NOVX activity, can be monitored in clinical trails of subjects exhibiting increased NOVX gene expression, protein levels, or upregulated NOVX activity. In such clinical trials, the expression or activity of NOVX and, preferably, other genes that have been implicated in, for example, a cellular proliferation or immune disorder can be used as a “read out” or markers of the immune responsiveness of a particular cell.

By way of example, and not of limitation, genes, including NOVX, that are modulated in cells by treatment with an agent (e.g., compound, drug or small molecule) that modulates NOVX activity (e.g., identified in a screening assay as described herein) can be identified. Thus, to study the effect of agents on cellular proliferation disorders, for example, in a clinical trial, cells can be isolated and RNA prepared and analyzed for the levels of expression of NOVX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOVX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of a NOVX protein, mRNA, or genomic DNA in the preadministration sample; (iii) obtaining one or more post-administration samples from the subject; (iv) detecting the level of expression or activity of the NOVX protein, mRNA, or genomic DNA in the post-administration samples; (v) comparing the level of expression or activity of the NOVX protein, mRNA, or genomic DNA in the pre-administration sample with the NOVX protein, mRNA, or genomic DNA in the post administration sample or samples; and (vi) altering the administration of the agent to the subject accordingly. For example, increased administration of the agent may be desirable to increase the expression or activity of NOVX to higher levels than detected, i.e., to increase the effectiveness of the agent. Alternatively, decreased administration of the agent may be desirable to decrease expression or activity of NOVX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant NOVX expression or activity. The disorders include but are not limited to, e.g., those diseases, disorders and conditions listed above, and more particularly include those diseases, disorders, or conditions associated with homologs of a NOVX protein, such as those summarized in Table A.

These methods of treatment will be discussed more fully, below.

Diseases and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOVX expression or activity can be identified by, for example, any or a combination of diagnostic or prognostic assays as described herein. Administration of a prophylactic agent can occur prior to the manifestation of symptoms characteristic of the NOVX aberrancy, such that a disease or disorder is prevented or, alternatively, delayed in its progression. Depending upon the type of NOVX aberrancy, for example, a NOVX agonist or NOVX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOVX expression or activity for therapeutic purposes. The modulatory method of the invention involves contacting a cell with an agent that modulates one or more of the activities of NOVX protein activity associated with the cell. An agent that modulates NOVX protein activity can be an agent as described herein, such as a nucleic acid or a protein, a naturally-occurring cognate ligand of a NOVX protein, a peptide, a NOVX peptidomimetic, or other small molecule. In one embodiment, the agent stimulates one or more NOVX protein activity. Examples of such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of a NOVX protein or nucleic acid molecule. In one embodiment, the method involves administering an agent (e.g., an agent identified by a screening assay described herein), or combination of agents that modulates (e.g., up-regulates or down-regulates) NOVX expression or activity. In another embodiment, the method involves administering a NOVX protein or nucleic acid molecule as therapy to compensate for reduced or aberrant NOVX expression or activity.

Stimulation of NOVX activity is desirable in situations in which NOVX is abnormally downregulated and/or in which increased NOVX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The NOVX nucleic acids and proteins of the invention are useful in potential prophylactic and therapeutic applications implicated in a variety of disorders. The disorders include but are not limited to, e.g., those diseases, disorders and conditions listed above, and more particularly include those diseases, disorders, or conditions associated with homologs of a NOVX protein, such as those summarized in Table A.

As an example, a cDNA encoding the NOVX protein of the invention may be useful in gene therapy, and the protein may be useful when administered to a subject in need thereof. By way of non-limiting example, the compositions of the invention will have efficacy for treatment of patients suffering from diseases, disorders, conditions and the like, including but not limited to those listed herein.

Both the novel nucleic acid encoding the NOVX protein, and the NOVX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies, which immunospecifically-bind to the novel substances of the invention for use in therapeutic or diagnostic methods.

The invention will be further described in the following examples, which do not limit the scope of the invention described in the claims.

### EXAMPLES

### Example A

### Polynucleotide and Polypeptide Sequences, and Homology Data

### Example 1

The NOV1 clone was analyzed, and the nucleotide and encoded polypeptide sequences are shown in Table 1A.

TABLE 1A
NOV1 Sequence Analysis

NOV1a,
CG103910-02 SEQ ID NO:1 1224 bp
DNA Sequence ORF Start: ATG at 1 ORF Stop: TGA at 1041
ATGCACGTGCGCTCACTGCGAGCTGCGGCGCCGCACAGCTTCGTGGCGCTCTGGGCACCCCTGTTCCT
GCTGCGCTCCGCCCTGGCCGACTTCAGCCTGGACAACGAGGTGCACTCGAGCTTCATCCACCGGCGCC
TCCGCAGCCAGGAGCGGCGGGAGATGCAGCGCGAGATCCTCTCCATTTTGGGCTTGCCCCACCGCCCG
CGCCCGCACCTCCAGGGCAAGCACAACTCGGCACCCATGTTCATGCTGGACCTGTACAACGCCATGGC
GGTGGAGGAGGGCGGCGGGCCCGGCGGCCAGGGCTTCTCCTACCCCTACAAGGCCGTCTTCAGTACCC
AGGGCCCCCCTCTGGCCAGCCTGCAAGATAGCCATTTCCTCACCGACGCCGACATGGTCATGAGCTTC
GTCAACCTCGTGGAACATGACAAGGAATTCTTCCACCCACGCTACCACCATCGAGAGTTCCGGTTTGA
TCTTTCCAAGATCCCAGAAGGGGAAGCTGTCACGGCAGCCGAATTCCGGATCTACAAGGACTACATCC
GGGAACGCTTCGACAATGAGACGTTCCGGATCAGCGTTTATCAGGTGCTCCAGGAGCACTTGGGCAGG
GAATCGGATCTCTTCCTGCTCGACAGCCGTACCCTCTGGGCCTCGGAGGAGGGCTGGCTGGTGTTTGA
CATCACAGCCACCAGCAACCACTGGGTGGTCAATCCGCGGCACAACCTGGGCCTGCAGCTCTCGGTGG
AGACGCTGGATGGGCAGAGCATCAACCCCAAGTTGGCGGGCCTGATTGGGCGGCACGGGCCCCAGAAC
AAGCAGCCCTTCATGGTGGCTTTCTTCAAGGCCACGGAGGTCCACTTCCGCAGCATCCGGTCCACGGG
GAGCAAACAGCGCAGCCAGAACCGCTCCAAGACGCCCAAGAACCAGGAAGCCCTGCGGATGGCCAACG
TGGCAGGTCCACTTCATCAACCCGGAAACGGTGCCCAAGCCCTGCTGTGCGCCCACGCAGCTCAATGC
CATCTCCGTCCTCTACTTCGATGACAGCTCCAACGTCATCCTGAAGAAATACAGAAACATGGTGGTCC
GGGCCTGTGGCTGCCACTAGCTCCTCCGAGAATTCAGACCCTTTGGGGCCAAGTTTTTCTGGATCCTC
CATTGCTCGCCTTGGCCAGGAACCAGCAGACCAACTGCCTTTTGTGAGACCTTCCCCTCCCTATCCCC
NOV1a,
CG103910-02
Protein Sequence SEQ ID NO: 2 347 aa MW at 39545.6 kD
MHVRSLRAAAPHSFVALWAPLFLLRSALADFSLDNEVHSSFIHRRLRSQERREMQREILSILGLPHRP
RPHLQGKHNSAPMFMLDLYNAMAVEEGGGPGGQGFSYPYKAVFSTQGPPLASLQDSHFLTDADMVMSF
VNLVEHDKEFFHPRYHHREFRFDLSKIPEGEAVTAAEFRIYKDYIRERFDNETFRISVYQVLQEHLGR
ESDLFLLDSRTLWASEEGWLVFDITATSNHWVVNPRHNLGLQLSVETLDGQSINPKLAGLIGRHGPQN
KQPFMVAFFKATEVHFRSIRSTGSKQRSQNRSKTPKNQEALRMANVAGPLHQPGNGAQALLCAHAAQC
HLRPLLR
NOV1b,
CG103910-03 SEQ ID NO: 3 1226 bp
DNA Sequence ORF Start: ATG at 1 ORF Stop: IGA at 976
ATGCACGTGCGCTCACTGCGAGCTGCGGCGCCGCACAGCTTCGTGGCGCTCTGGGCACCCCTGTTCCT
GCTGCGCTCCGCCCTGGCCGACTTCAGCCTCGACAACGAGGTGCACTCGAGCTTCATCCACCGGCGCC
TCCGCAGCCAGGAGCGGCGGGAGATGCAGCGCGAGATCCTCTCCATTTTGGGCTTGCCCCACCGCCCG
CGCCCGCACCTCCAGGGCAAGCACAACTCGGCACCCATGTTCATGCTGGACCTGTACAACGCCATGGC
GGTGGAGGAGGGCGGCGGGCCCGGCGGCCAGGGCTTCTCCTACCCCTACAAGGCCGTCTTCAGTACCC
AGGGCCCCCCTCTGGCCAGCCTGCAAGATAGCCATTTCCTCACCGACGCCGACATGGTCATGAGCTTC
GTCAACCTCGTGGAACATGACAAGGAATTCTTCCACCCACGCTACCACCATCGAGAGTTCCGGTTTGA
TCTTTCCAAGATCCCAGAAGGGGAAGCTGTCACGGCAGCCGAATTCCGGATCTACAAGGACTACATCC
GGGAACGCTTCGACAATGAGACGTTCCGGATCAGCGTTTATCAGGTGCTCCAGGAGCACTTGGGCAGG
GAATCGGATCTCTTCCTGCTCGACAGCCGTACCCTCTGGGCCTCGGAGGAGGGCTGGCTGGTGTTTGA
CATCACAGCCACCAGCAACCACTGGGTGGTCAATCCGCGGCACAACCTGGGCCTGCAGCTCTCGGTGG
AGACGCTGGATGGGCAGAGCATCAACCCCAAGTTGGCGGGCCTGATTGGGCGGCACGGGCCCCAGAAC
AAGCAGCCCTTCATGGTGGCTTTCTTCAAGGCCACGGAGGTCCACTTCCGCAGCATCCGGTCCACGGG
GAGCAAACAGCGCAGCCAGAACCGCTCCAAGACGCCCAAGAACCAGGAAGCCCTGCGGATGGCCAACG
TGGCAGGACTGGATCATCGCGCCTGAAGGCTACGCCGCCTACTACTGTGAGGGGGAGTGTGCCTTCCC
TCTGAACTCCTACATGAACGCCACCAACCACGCCATCGTGCAGACGCTGGTCCACTTCATCAACCCGG
AAACGGTGCCCAAGCCCTGCTGTGCGCCCACGCAGCTCTATGCCATCTCCGTCCTCTACTTCGATGAC
AGTTCCAACGTCATCCTGAAGAAATACAGATACATGGTGGTCCGGGCCTGTGGCTGCCACTAGCTCCT
CC
NOV1b,
CG103910-03
Protein Sequence SEQ ID NO: 4 325 aa MW at 37269.9 kD
MHVRSLRAAAPHSFVALWAPLFLLRSALADFSLDNEVHSSFIHRRLRSQERREMQREILSILGLPHRP
RPHLQGKHNSAPMFMLDLYNAMAVEEGGGPGGQGFSYPYKAVFSTQGPPLASLQDSHFLTDADMVMSF
VNLVEHDKEFFHPRYHHREFRFDLSKIPEGEAVTAAEFRIYKDYIRERFDNETFRISVYQVLQEHLGR
ESDLFLLDSRTLWASEEGWLVFDITATSNHWVVNPRHNLGLQLSVETLDGQSINPKLAGLIGRHGPQN
KQPFMVAFFKATEVHFRSIRSTGSKQRSQNRSKTPKNQEALRMANVAGLDHRA
NOV1c,
CG103910-O1 SEQ ID NO: 5 1878 bp
DNA Sequence ORF Start: ATG at 123 ORF Stop: TAG at 1418
GGGCGCAGCGGGGCCCGTCTGCAGCAAGTGACCGACGGCCGGGACGGCCGCCTGCCCCCTCTGCCACC
TGGGGCGGTGCGGGCCCGGAGCCCGGAGCCCGGGTAGCGCGTAGAGCCGGCGCGATGCACGTGCGCTC
ACTGCGAGCTGCGGCGCCGCACAGCTTCGTGGCGCTCTGGGCACCCCTGTTCCTGCTGCGCTCCGCCC
TGGCCGACTTCAGCCTGGACAACGAGGTGCACTCGAGCTTCATCCACCGGCGCCTCCGCAGCCAGGAG
CGGCGGGAGATGCAGCGCGAGATCCTCTCCATTTTGGGCTTGCCCCACCGCCCGCGCCCGCACCTCCA
GGGCAAGCACAACTCGGCACCCATGTTCATGCTGGACCTGTACAACGCCATGGCGGTGGAGGAGGGCG
GCGGGCCCGGCGGCCAGGGCTTCTCCTACCCCTACAAGGCCGTCTTCAGTACCCAGGGCCCCCCTCTG
GCCAGCCTGCAAGATAGCCATTTCCTCACCGACGCCGACATGGTCATGAGCTTCGTCAACCTCGTGGA
ACATGACAAGGAATTCTTCCACCCACGCTACCACCATCGAGAGTTCCGGTTTGATCTTTCCAAGATCC
CAGAAGGGGAAGCTGTCACGGCAGCCGAATTCCGGATCTACAAGGACTACATCCGGGAACGCTTCGAC
AATGAGACGTTCCGGATCAGCGTTTATCAGGTGCTCCAGGAGCACTTGGGCAGGGAATCGGATCTCTT
CCTGCTCGACAGCCGTACCCTCTGGGCCTCGGAGGAGGGCTGGCTGGTGTTTGACATCACAGCCACCA
GCAACCACTGGGTGGTCAATCCGCGGCACAACCTGGGCCTGCAGCTCTCGGTGGAGACGCTGGATGGG
CAGAGCATCAACCCCAAGTTGGCGGGCCTGATTGGGCGGCACGGGCCCCAGAACAAGCAGCCCTTCAT
GGTGGCTTTCTTCAAGGCCACGGAGGTCCACTTCCGCAGCATCCGGTCCACGGGGAGCAAACAGCGCA
GCCAGAACCGCTCCAAGACGCCCAAGAACCAGGAAGCCCTGCGGATGGCCAACGTGGCAGAGAACAGC
AGCAGCGACCAGAGGCAGGCCTGTAAGAAGCACGAGCTGTATGTCAGCTTCCGAGACCTGGGCTGGCA
GGACTGGATCATCGCGCCTGAAGGCTACGCCGCCTACTACTGTGAGGGGGAGTGTGCCTTCCCTCTGA
ACTCCTACATGAACGCCACCAACCACGCCATCGTGCAGACGCTGGTCCACTTCATCAACCCGGAAACG
GTGCCCAAGCCCTGCTGTGCGCCCACGCAGCTCAATGCCATCTCCGTCCTCTACTTCGATGACAGCTC
CAACGTCATCCTGAAGAAATACAGAAACATGGTGGTCCGGGCCTGTGGCTGCCACTAGCTCCTCCGAG
AATTCAGACCCTTTGGGGCCAAGTTTTTCTGGATCCTCCATTGCTCGCCTTGGCCAGGAACCAGCAGA
CCAACTGCCTTTTGTGAGACCTTCCCCTCCCTATCCCCAACTTTAAAGGTGTGAGAGTATTAGGAAAC
ATGAGCAGCATATGGCTTTTGATCAGTTTTTCAGTGGCAGCATCCAATGAACAAGATCCTACAAGCTG
TGCAGGCAAAACCTAGCAGGAAAAAAAAACAACGCATAAAGAAAAATGGCCGGGCCAGGTCATTGGCT
GGGAAGTCTCAGCCATGCACGGACTCGTTTCCAGAGGTAATTATGAGCGCCTACCAGCCAGGCCACCC
AGCCGTGGGAGGAAGGGGGCGTGGCAAGGGGTGGGCACATTGGTGTCTGTGCGAAAGGAAAATTGACC
CGGAAGTTCCTGTAATAAATGTCACAATAAAACGAATGAATG
NOV1c,
CG103910-O1
Protein Sequence SEQ NO: 6 431 aa MW at 49312.4 kD
MHVRSLRAAAPHSFVALWAPLFLLRSALADFSLDNEVHSSFIHRRLRSQERREMQREILSILGLPHRP
RPHLQGKHNSAPMFMLDLYNAMAVEEGGGPGGQGFSYPYKAVFSTQGPPLASLQDSHFLTDADMVMSF
VNLVEHDKEFFHPRYHHREFRFDLSKIPEGEAVTAAEFRIYKDYIRERFDNETFRISVYQVLQEHLGR
ESDLFLLDSRTLWASEEGWLVFDITATSNHWVVNPRHNLGLQLSVETLDGQSINPKLAGLIGRHGPQN
KQPFMVAFFKATEVHFRSIRSTGSKQRSQNRSKTPKNQEALRMANVAENSSSDQRQACKKHELYVSFR
DLGWQDWIIAPEGYAAYYCEGECAFPLNSYMNATNHAIVQTLVHFINPETVPKPCCAPTQLNAISVLY
FDDSSNVILKKYRNMVVRACGCH
NOV1d,
CG103910-04 SEQ ID NO: 7 997 bp
DNA Sequence ORF Start: ATG at 14 ORF Stop: end of sequence
CACCGGATCCACCATGCACGTGCGCTCACTGCGAGCTGCGGCGCCGCACAGCTTCGTGGCGCTCTGGG
CACCCCTGTTCCTGCTGCGCTCCGCCCTGGCCGACTTCAGCCTGGACAACGAGGTGCACTCGAGCTTC
ATCCACCGGCGCCTCCGCAGCCAGGAGCGGCGGGAGATGCAGCGCGAGATCCTCTCCATTTTGGGCTT
GCCCCACCGCCCGCGCCCGCACCTCCAGGGCAAGCACAACTCGGCACCCATGTTCATGCTGGACCTGT
ACAACGCCATGGCGGTGGAGGAGGGCGGCGGGCCCGGCGGCCAGGGCTTCTCCTACCCCTACAAGGCC
GTCTTCAGTACCCAGGGCCCCCCTCTGGCCAGCCTGCAAGATAGCCATTTCCTCACCGACGCCGACAT
GGTCATGAGCTTCGTCAACCTCGTGGAACATGACAAGGAATTCTTCCACCCACGCTACCACCATCGAG
AGTTCCGGTTTGATCTTTCCAAGATCCCAGAAGGGGAAGCTGTCACGGCAGCCGAATTCCGGATCTAC
AAGGACTACATCCGGGAACGCTTCGACAATGAGACGTTCCGGATCAGCGTTTATCAGGTGCTCCAGGA
GCACTTGGGCAGGGAATCGGATCTCTTCCTGCTCGACAGCCGTACCCTCTGGGCCTCGGAGGAGGGCT
GGCTGGTGTTTGACATCACAGCCACCAGCAACCACTGGGTGGTCAATCCGCGGCACAACCTGGGCCTG
CAGCTCTCGGTGGAGACGCTGGATGGGCAGAGCATCAACCCCAAGTTGGCGGGCCTGATTGGGCGGCA
CGGGCCCCAGAACAAGCAGCCCTTCATGGTGGCTTTCTTCAAGGCCACGGAGGTCCACTTCCGCAGCA
TCCGGTCCACGGGGAGCAAACAGCGCAGCCAGAACCGCTCCAAGACGCCCAAGAACCAGGAAGCCCTG
CGGATGGCCAACGTGGCAGGACTGGATCATCGCGCC
NOV1d,
CG103910-04
Protein Sequence SEQ ID NO:8 325 aa MW at 37269.9 kD
MHVRSLRAAAPHSFVALWAPLFLLRSALADFSLDNEVHSSFIHRRLRSQERREMQREILSILGLPHRP
RPHLQGKHNSAPMFMLDLYNAMAVEEGGGPGGQGFSYPYKAVFSTQGPPLASLQDSHFLTDADMVMSF
VNLVEHDKEFFHPRYHHREFRFDLSKIPEGEAVTAAEFRIYKDYIRERFDNETFRISVYQVLQEHLGR
ESDLFLLDSRTLWASEEGWLVFDITATSNHWVVNPRHNLGLQLSVETLDGQSINPKLAGLIGRHGPQN
KQPFMVAFFKATEVHFRSIRSTGSKQRSQNRSKTPKNQEALRMANVAGLDHRA
NOV1e, 13382317
SNP CG103910-02 SEQ ID NO: 9 SNP at position 1193
DNA Sequence ORF Start: ATG at 1 ORF Stop: TGA at 1042
ATGCACGTGCGCTCACTGCGAGCTGCGGCGCCGCACAGCTTCGTGGCGCTCTGGGCACCCCTGTTCCTGC
TGCGCTCCGCCCTGGCCGACTTCAGCCTGGACAACGAGGTGCACTCGAGCTTCATCCACCGGCGCCTCCG
CAGCCAGGAGCGGCGGGAGATGCAGCGCGAGATCCTCTCCATTTTGGGCTTGCCCCACCGCCCGCGCCCG
CACCTCCAGGGCAAGCACAACTCGGCACCCATGTTCATGCTGGACCTGTACAACGCCATGGCGGTGGAGG
AGGGCGGCGGGCCCGGCGGCCAGGGCTTCTCCTACCCCTACAAGGCCGTCTTCAGTACCCAGGGCCCCCC
TCTGGCCAGCCTGCAAGATAGCCATTTCCTCACCGACGCCGACATGGTCATGAGCTTCGTCAACCTCGTG
GAACATGACAAGGAATTCTTCCACCCACGCTACCACCATCGAGAGTTCCGGTTTGATCTTTCCAAGATCC
CAGAAGGGGAAGCTGTCACGGCAGCCGAATTCCGGATCTACAAGGACTACATCCGGGAACGCTTCGACAA
TGAGACGTTCCGGATCAGCGTTTATCAGGTGCTCCAGGAGCACTTGGGCAGGGAATCGGATCTCTTCCTG
CTCGACAGCCGTACCCTCTGGGCCTCGGAGGAGGGCTGGCTGGTGTTTGACATCACAGCCACCAGCAACC
ACTGGGTGGTCAATCCGCGGCACAACCTGGGCCTGCAGCTCTCGGTGGAGACGCTGGATGGGCAGAGCAT
CAACCCCAAGTTGGCGGGCCTGATTGGGCGGCACGGGCCCCAGAACAAGCAGCCCTTCATGGTGGCTTTC
TTCAAGGCCACGGAGGTCCACTTCCGCAGCATCCGGTCCACGGGGAGCAAACAGCGCAGCCAGAACCGCT
CCAAGACGCCCAAGAACCAGGAAGCCCTGCGGATGGCCAACGTGGCAGGTCCACTTCATCAACCCGGAAA
CGGTGCCCAAGCCCTGCTGTGCGCCCACGCAGCTCAATGCCATCTCCGTCCTCTACTTCGATGACAGCTC
CAACGTCATCCTGAAGAAATACAGAAACATGGTGGTCCGGGCCTGTGGCTGCCACTAGCTCCTCCGAGAA
TTCAGACCCTTTGGGGCCAAGTTTTTCTGGATCCTCCATTGCTCGCCTTGGCCAGGAACCAGCAGACCAA
CTCCCTTTTGTGAGACCTTCCCCTCCCTATCCCC
NOV1e, 13382317
SNP CG103910-02 SNP: No change in Protein
Protein Sequence SEQ ID NO: 10 325 aa sequence
MHVRSLRAAAPHSFVALWAPLFLLRSALADFSLDNEVHSSFIHRRLRSQERREMQREILSILGLPHRP
RPHLQGKHNSAPMFMLDLYNAMAVEEGGGPGGQGFSYPYKAVFSTQGPPLASLQDSHFLTDADMVMSF
VNLVEHDKEFFHPRYHHREFRFDLSKIPEGEAVTAAEFRIYKDYIRERFDNETFRISVYQVLQEHLGR
ESDLFLLDSRTLWASEEGWLVFDITATSNHWVVNPRHNLGLQLSVETLDGQSINPKLAGLIGRHGPQN
KQPFMVAFFKATEVHFRSIRSTGSKQRSQNRSKTPKNQEALRMANVAGPLHQPGNGAQALLCAHAAQC
HLRPLLR

A ClustalW comparison of the above protein sequences yields the following sequence alignment shown in Table 1B.

TABLE 1B
Comparison of the NOV1 protein sequences.

| | |
|---|---|
| NOV1a | MHVRSLRAAAPHSFVALWAPLFLLRSALADFSLDNEVHSSFIHRRLRSQERREMQREILS |
| NOV1b | MHVRSLRAAAPHSFVALWAPLFLLRSALADFSLDNEVHSSFIHRRLRSQERREMQREILS |
| NOV1c | MHVRSLRAAAPHSFVALWAPLFLLRSALADFSLDNEVHSSFIHRRLRSQERREMQREILS |
| NOV1d | MHVRSLRAAAPHSFVALWAPLFLLRSALADFSLDNEVHSSFIHRRLRSQERREMQREILS |
| NOV1a | ILGLPHRPRPHLQGKHNSAPMFMLDLYNAMAVEEGGGPGGQGFSYPYKAVFSTQGPPLAS |
| NOV1b | ILGLPHRPRPHLQGKHNSAPMFMLDLYNAMAVEEGGGPGGQGFSYPYKAVFSTQGPPLAS |
| NOV1c | ILGLPHRPRPHLQGKHNSAPMFMLDLYNAMAVEEGGGPGGQGFSYPYKAVFSTQGPPLAS |
| NOV1d | ILGLPHRPRPHLQGKHNSAPMFMLDLYNAMAVEEGGGPGGQGFSYPYKAVFSTQGPPLAS |
| NOV1a | LQDSHFLTDADMVMSFVNLVEHDKEFFHPRYHHREFRFDLSKIPEGEAVTAAEFRIYKDY |
| NOV1b | LQDSHFLTDADMVMSFVNLVEHDKEFFHPRYHHREFRFDLSKIPEGEAVTAAEFRIYKDY |
| NOV1c | LQDSHFLTDADMVMSFVNLVEHDKEFFHPRYHHREFRFDLSKIPEGEAVTAAEFRIYKDY |
| NOV1d | LQDSHFLTDADMVMSFVNLVEHDKEFFHPRYHHREFRFDLSKIPEGEAVTAAEFRIYKDY |
| NOV1a | IRERFDNETFRISVYQVLQEHLGRESDLFLLDSRTLWASEEGWLVFDITATSNHWVVNPR |
| NOV1b | IRERFDNETFRISVYQVLQEHLGRESDLFLLDSRTLWASEEGWLVFDITATSNHWVVNPR |
| NOV1c | IRERFDNETFRISVYQVLQEHLGRESDLFLLDSRTLWASEEGWLVFDITATSNHWVVNPR |
| NOV1d | IRERFDNETFRISVYQVLQEHLGRESDLFLLDSRTLWASEEGWLVFDITATSNHWVVNPR |
| NOV1a | HNLGLQLSVETLDGQSINPKLAGLIGRHGPQNKQPFMVAFFKATEVHFRSIRSTGSKQRS |
| NOV1b | HNLGLQLSVETLDGQSINPKLAGLIGRHGPQNKQPFMVAFFKATEVHFRSIRSTGSKQRS |
| NOV1c | HNLGLQLSVETLDGQSINPKLAGLIGRHGPQNKQPFMVAFFKATEVHFRSIRSTGSKQRS |
| NOV1D | HNLGLQLSVETLDGQSINPKLAGLIGRHGPQNKQPFMVAFFKATEVHFRSIRSTGSKQRS |
| NOV1a | QNRSKTPKNQEALRMANVAG----PLHQPGN---------------------GAQALLCA |
| NOV1b | QNRSKTPKNQEALRMANVAG----LDHRA------------------------------- |
| NOV1c | QNRSKTPKNQEALRMANVAENSSSDQRQACKKHELYVSFRDLGWQDWIIAPEGYAAYYCE |
| NOV1d | QNRSKTPKNQEALRMANVAG---LDHRA-------------------------------- |
| NOV1a | HAAQCHLRPLLR------------------------------------------------ |
| NOV1b | ------------------------------------------------------------ |
| NOV1c | GECAFPLNSYMNATNHAIVQTLVHFINPETVPKPCCAPTQLNAISVLYFDDSSNVILKKY |
| NOV1d | ------------------------------------------------------------ |
| NOV1a | ----------- |
| NOV1b | ----------- |
| NOV1c | RNMVVRACGCH |
| NOV1d | ----------- |
| NOV1a | (SEQ ID NO: 2) |
| NOV1b | (SEQ ID NO: 4) |
| NOV1c | (SEQ ID NO: 6) |
| NOV1d | (SEQ ID NO: 8) |

Further analysis of the NOV1a protein yielded the following properties shown in Table 1C.

TABLE 1C
Protein Sequence Properties NOV1a

| SignalP | Cleavage site between residues 30 and 31 | | | |
|---|---|---|---|---|
| analysis: | | | | |
| PSORT II | PSG: a new signal peptide prediction method | | | |
| analysis: | N-region: length 7; pos. chg 2; neg. chg 0 | | | |
| | H-region: length 17; peak value 9.51 | | | |
| | PSG score: 5.11 | | | |
| | GvH: von Heijne's method for signal seq. recognition | | | |
| | GvH score (threshold: −2.1): 0.94 | | | |
| | possible cleavage site: between 29 and 30 | | | |
| | >>> Seems to have a cleavable signal peptide (1 to 29) | | | |
| | ALOM: Klein et al's method for TM region allocation | | | |
| | Init position for calculation: 30 | | | |
| | Tentative number of TMS(s) for the threshold 0.5: 0 | | | |
| | number of TMS(s) . . . fixed | | | |
| | PERIPHERAL Likelihood = 6.10 (at 124) | | | |
| | ALOM score: 6.10 (number of TMSs: 0) | | | |
| | MTOP: Prediction of membrane topology (Hartmann et al.) | | | |
| | Center position for calculation: 14 | | | |
| | Charge difference: −5.5 C(−1.5)-N(4.0) | | | |
| | N >= C: N-terminal side will be inside | | | |
| | MITDISC: discrimination of mitochondrial targeting seq | | | |
| | R content: | 3 | Hyd Moment (75): | 6.00 |
| | Hyd Moment(95): | 9.57 | G content: | 0 |
| | D/E content: | 1 | S/T content: | 3 |

Score: −0.96
Gavel: prediction of cleavage sites for mitochondrial preseq
R-2 motif at 35 LRS|AL
NUCDISC: discrimination of nuclear localization signals
pat4: none
pat7: none
bipartite: none
content of basic residues: 11.5%
NLS Score: −0.47
KDEL: ER retention motif in the C-terminus: none
ER Membrane Retention Signals:
XXRR-like motif in the N-terminus: HVRS
none
SK-L: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrpsines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 70.6
COIL: Lupas's algorithm to detect coiled-coil regions
total: 0 residues
Final Results (k = 9/23):
44.4%: extracellular, including cell wall
22.2%: Golgi
11.1%: vacuolar
11.1%: nuclear
11.1%: endoplasmic reticulum
>> prediction for CG103910-02 is exc (k = 9)

A search of the NOV1a protein against the Geneseq database, a proprietary database that contains sequences published in patents and patent publication, yielded several homologous proteins shown in Table 1D.

TABLE 1D
Geneseq Results for NOV1a

| | | NOV1a | Identities/ | |
|---|---|---|---|---|
| | | Residues/ | Similarities | |
| Geneseq | Protein/Organism/Length | Match | for the | Expect |
| Identifier | [Patent#, Date] | Residues | Matched Region | Value |
| ABU56730 | Lung cancer-associated polypeptide | 1 . . . 319 | 319/319 (100%) | 0.0 |
| | #323 - Unidentified, 431 aa. | 1 . . . 319 | 319/319 (100%) | |
| | [WO200286443-A2, 31 OCT. 2002] | | | |
| AAU97017 | Human osteogenic protein (OP-1) - | 1 . . . 319 | 319/319 (100%) | 0.0 |
| | Homo sapiens, 431 aa. | 1 . . . 319 | 319/319 (100%) | |
| | [US2002049159-A1, 25 APR. 2002] | | | |
| AAE25993 | Human osteogenic protein 1 (hOP- | 1 . . . 319 | 319/319 (100%) | 0.0 |
| | 1) - Homo sapiens, 431 aa. | 1 . . . 319 | 319/319 (100%) | |
| | [US6407060-B1, 18 JUN. 2002] | | | |
| ABB82416 | Human osteogenic protein-1 (OP-1) - | 1 . . . 319 | 319/319 (100%) | 0.0 |
| | Homo sapiens, 43 1 aa. | 1 . . . 319 | 319/319 (100%) | |
| | [WO200270029-A2, 12 SEP. 2002] | | | |
| AAB37614 | Human OP-1 - Homo sapiens, 431 | 1 . . . 319 | 319/319 (100%) | 0.0 |
| | aa. [WO200066620-A2, 09 NOV. | 1 . . . 319 | 319/319 (100%) | |
| | 2000] | | | |

In a BLAST search of public sequence databases, the NOV1a protein was found to have homology to the proteins shown in the BLASTP data in Table 1E.

TABLE 1E
Public BLASTP Results for NOV1a

| | | NOV1a | Identities/ | |
|---|---|---|---|---|
| Protein | | Residues/ | Similarities | |
| Accession | | Match | for the | Expect |
| Number | Protein/Organism/Length | Residues | Matched Portion | Value |
| Q9BTB3 | Similar to bone morphogenetic | 1 . . . 319 | 319/319 (100%) | 0.0 |
| | protein 7 (Osteogenic protein 1) - | 1 . . . 319 | 319/319 (100%) | |
| | Homo sapiens (Human), 412 aa. | | | |
| P18075 | Bone morphogenetic protein 7 | 1 . . . 319 | 319/319 (100%) | 0.0 |
| | precursor (BMP-7) (Osteogenic | 1 . . . 319 | 319/319 (100%) | |
| | protein 1) (OP-1) - Homo sapiens | | | |
| | (Human), 431 aa. | | | |
| P23359 | Bone morphogenetic protein 7 | 1 . . . 319 | 309/319 (96%) | e−180 |
| | precursor (BMP-7) (Osteogenic | 1 . . . 318 | 313/319 (97%) | |
| | protein 1) (OP-1) - Mus musculus | | | |
| | (Mouse), 430 aa. | | | |
| JQ1184 | osteogenic protein 1 precursor - | 1 . . . 319 | 308/319 (96%) | e−179 |
| | mouse, 430 aa. | 1 . . . 318 | 312/319 (97%) | |
| Q9I8T6 | Bone morphogenetic protein 7 - | 39 . . . 319 | 246/285 (86%) | e−143 |
| | Gallus gallus (Chicken), 398 aa | 2 . . . 286 | 264/285 (92%) | |
| | (fragment). | | | |

PFam analysis predicts that the NOV1a protein contains the domains shown in the Table 1F.

TABLE 1F
Domain Analysis of NOV1a

| | | Identities/ | |
|---|---|---|---|
| | NOV1a | Similarities | |
| | Match | for the | Expect |
| Pfam Domain | Region | Matched Region | Value |
| TGFb_propeptide | 37 . . . 281 | 104/269 (39%) | 3e−100 |
| | | 223/269 (83%) | |

### Example 2

The NOV2 clone was analyzed, and the nucleotide and encoded polypeptide sequences are shown in Table 2A.

TABLE 2A
NOV2 Sequence Analysis

NOV2a,
CG106298-02 SEQ ID NO: 11 1162 bp
DNA Sequence ORF Start: ATG at 19 ORF Stop: TGA at 832
CTACTGAGAATATCTAACATGTTGTTACTAATCAATGTCATTCTGACCTTGTGGGTTTCCTGTGCTAA
TGGACAAGTGAAACCTTGTGATTTTCCAGACATTAAACATGGAGGTCTATTTCATGAGAATATGCGTA
GACCATACTTTCCAGTAGCTGTAGGACAATCTTACTCCTATTACTGTGACCAAAATTTTGTGACTCCT
TCAGGAAGTTACTGGGATTACATTCACTGCACACAAGATGGGTGGTTGCCAACAGTCCCATGCCTCAG
AACATGCTCAAAATCAGATATAGAAATTGAAAATGGATTCATTTCTGAATCTTCCTCTATTTATATTT
TAAATAAAGAAATACAATATAAATGTAAACCAGGATATGCAACAGCAGATGGAAATTCTTCAGGTTCA
ATTACATGTTTGCAAAATGGATGGTCAGCACAACCAATTTGCATTAAATTTTGTGATATGCCTGTTTT
TGAGAATTCCAGAGCCAAGAGTAATGGCATGCGGTTTAAGCTCCATGACACATTGGACTACGAATGCT
ACGATGGATATGAAATCAGTTATGGAAACACCACAGGTTCCATAGTGTGTGGTGAAGATGGGTGGTCC
CATTTCCCAACATGTTATAATTCTTCAGAAAAGTGTGGGCCTCCTCCACCTATTAGCAATGGTGATAC
CACCTCCTTTCTACTAAAAGTGTATGTGCCACAGTCAAGAGTCGAGTACCAATGCCAGTCCTACTATG
AACTTCAGGGTTCTAATTATGTAACATGTAGTAATGGAGAGTGGTCGGAACCACCAAGATGCATACGT
ATCCACTTCTGCAGATGATCATGTCCAAGTTTGAGCTCCAAACTATGCAAGTGGCAAGACTGAAGAAG
AAATTAGTATCCTCAAATCAAAATAGTTTACAAGTATCTTCAAACTTGATTTCATAGAAAAGTGTTAG
GTTTCAGAGATAAATTCTGAGTCTCAAATTTGATTGAATGGGGAGATGGACACTCCTAAGATGGGTTT
CACAGCAAAAGCATTACCTCTTCTCACAATCAAGAACAGGAAAGGATTATAATTATCTGAAGTATAAG
ATCAGTTCCATGATACAAGCAAGACTTTCAGTCTTCAAAACTAAAGAAGCAAAGAGCATTCAAGCACA
GAATTC
NOV2a,
CG106298-02
Protein Sequence SEQ ID NO: 12 271 aa MW at 30635.1 kD
MLLLINVILTLWVSCANGQVKPCDFPDIKHGGLFHENMRRPYFPVAVGQSYSYYCDQNFVTPSGSYWD
YIHCTQDGWLPTVPCLRTCSKSDIEIENGFISESSSIYILNKEIQYKCKPGYATADGNSSGSITCLQN
GWSAQPICIKFCDMPVFENSRAKSNGMRFKLHDTLDYECYDGYEISYGNTTGSIVCGEDGWSHFPTCY
NSSEKCGPPPPI SNGDTTSFLLKVYVPQSRVEYQCQSYYELQGSNYVTCSNGEWSEPPRCIRIHFCR
NOV2b,
CG106298-01 SEQ ID NO: 13 12033 bp
DNA Sequence ORF Start: ATG at 78 ORF TAA at 1812
AATAATAATGAAAGATTTCAAACCCCAAACAGTGCAACTGAAACTTTTGCATTACTATACTACTGAGA
ATATCTAACATGTTGTTACTAATCAATGTCATTCTGACCTTGTGGGTTTCCTGTGCTAATGGACAAGA
AGTGAAACCTTGTGATTTTCCAGAAATTCAACATGGAGGTCTATATTATAAGAGTTTGCGTAGACTAT
ACTTTCCAGCAGCTGCAGGACAATCTTATTCCTATTACTGTGATCAAAATTTTGTGACTCCTTCAGGA
AGTTACTGGGATTACATTCATTGCACACAAGATGGTTGGTCACCAACGGTCCCATGCCTCAGAACATG
CTCAAAATCAGATGTAGAAATTGAAAATGGATTCATTTCTGAATCTTCCTCTATTTATATTTTAAATG
AAGAAACACAATATAATTGTAAACCAGGATATGCAACAGCAGATGGAAATTCTTCAGGATCAATTACA
TGTTTGCAAAATGGATGGTCAACACAACCAATTTGCATTAAATTTTGTGATATGCCTGTTTTTGAGAA
TTCCAGAGCCAAGAGTAATGGCATGTGGTTTAAGCTCCATGACACATTGGACTATGAATGCTATGATG
GATATGAAAGCAGTTATGGAAACACCACAGATTCCATAGTGTGTGGTGAAGATGGCTGGTCCCATTTG
CCAACATGCTATAATTCTTCAGAAAGCTGTGGGCCTCCTCCACCTATTAGCAATGGAGATACCACGTC
CTTCCCGCAAAAAGTGTATCTGCCATGGTCAAGAGTCGAGTACCAGTGCCAGTCCTACTATGAACTTC
AGGGTTCTAAATATGTAACATGTAGTAATGGAGACTGGTCAGAACCACCAAGATGCATATCAATGAAA
CCTTGTGAGTTTCCAGAAATTCAACATGGACATCTATATTATGAGAATACGCGTAGACCATACTTTCC
AGTAGCTACAGGACAATCTTACTCCTATTACTGTGACCAAAATTTTGTGACTCCTTCAGGAAGTTACT
GGGATTACATTCACTGCACACAAGATGGGTGGTTGCCAACAGTCCCATGCCTCAGAACATGCTCAAAA
TCAGATATAGAAATTGAAAATGGATTCATTTCTGAATCTTCCTCTATTTATATTTTAAATAAAGAAAT
ACAATATAAATGTAAACCAGGATATGCAACAGCAGATGGAAATTCTTCAGGTTCAATTACATGTTTGC
AAAATGGATGGTCAGCACAACCAATTTGCATTAAATTTTGTGATATGCCTGTTTTTGAGAATTCCAGA
GCCAAGAGTAATGGCATGCGGTTTAAGCTCCATGACACATTGGACTACGAATGCTACGATGGATATGA
AATCAGTTATGGAAACACCACAGGTTCCATAGTGTGTGGTGAAGATGGGTGGTCCCATTTCCCAACAT
GTTATAATTCTTCAGAAAAGTGTGGGCCTCCTCCACCTATTAGCAATGGTGATACCACCTCCTTTCTA
CTAAAAGTGTATGTGCCACAGTCAAGAGTCGAGTACCAATGCCAGTCCTACTATGAACTTCAGGGTTC
TAATTATGTAACATGTAGTAATGGAGAGTGGTCGGAACCACCAAGATGCATACATCCATGTATAATAA
CTGAAGAAAACATGAATAAAAATAACATACAGTTAAAAGGAAAAAGTGACATAAAATATTATGCAAAA
ACAGGGGATACCATTGAATTTATGTGTAAATTGGGATATAATGCGAATACATCAGTTCTATCATTTCA
AGCAGTGTGTAGGGAAGGCATAGTGGAATACCCCAGATGCGAATAAGGCAGCATTGTTACCCTAAATG
TATGTCCAACTTCCACTTCTCACTCTTATGGTCTCAAAGCTTGCAAAGATAGCTTCTGATATTGTTGT
AATTTCTACTTTATTTCAAAGAAAATTAATATAATAGTTTCAATTTGCAACTTAATATGTTCTCAAAA
ATATGTTAAAACAAACTAAATTATTGCTTATGCTTGTACTAAAATAATAAAAACTACCCTT
NOV2b,
CG106298-O1
Protein Sequence SEQ ID NO: 14 578 aa MW at 65309.0 kD
MLLLINVILTLWVSCANGQEVKPCDFPEIQHGGLYYKSLRRLYFPAAAGQSYSYYCDQNFVTPSGSYW
DYIHCTQDGWSPTVPCLRTCSKSDVEIENGFISESSSIYILNEETQYNCKPGYATADGNSSGSITCLQ
NGWSTQPICIKFCDMPVFENSRAKSNGMWFKLHDTLDYECYDGYESSYGNTTDSIVCGEDGWSHLPTC
YNSSESCGPPPPISNGDTTSFPQKVYLPWSRVEYQCQSYYELQGSKYVTCSNGDWSEPPRCISMKPCE
FPEIQHGHLYYENTRRPYFPVATGQSYSYYCDQNFVTPSGSYWDYIHCTQDGWLPTVPCLRTCSKSDI
EIENGFISESSSIYILNKEIQYKCKPGYATADGNSSGSITCLQNGWSAQPICIKFCDMPVFENSRAKS
NGMRFKLHDTLDYECYDGYEISYGNTTGSIVCGEDGWSHFPTCYNSSEKCGPPPPISNGDTTSFLLKV
YVPQSRVEYQCQSYYELQGSNYVTCSNGEWSEPPRCIHPCIITEENMNKNNIQLKGKSDIKYYAKTGD
TIEFMCKLGYNANTSVLSFQAVCREGIVEYPRCE

A ClustalW comparison of the above protein sequences yields the following sequence alignment shown in Table 2B.

TABLE 2B
Comparison of the NOV2 protein sequences.

| | |
|---|---|
| NOV2a | MLLLINVILTLWVSCANGQ-VKPCDFPDIKHGGLFHENMRRPYFPVAVGQSYSYYCDQNF |
| NOV2b | MLLLINVILTLWVSCANGQEVKPCDFPEIQHGGLYYKSLRRLYFPAAAGQSYSYYCDQNF |
| NOV2a | VTPSGSYWDYIHCTQDGWLPTVPCLRTCSKSDIEIENGFISESSSIYILNKEIQYKCKPG |
| NOV2b | VTPSGSYWDYIHCTQDGWSPTVPCLRTCSKSDVEIENGFISESSSIYILNEETQYNCKPG |
| NOV2a | YATADGNSSGSITCLQNGWSAQPICIKFCDMPVFENSRAKSNGMRFKLHDTLDYECYDGY |
| NOV2b | YATADGNSSGSITCLQNGWSTQPICIKFCDMPVFENSRAKSNGMWFKLHDTLDYECYDGY |
| NOV2a | EISYGNTTGSIVCGEDGWSHFPTCYNSSEKCGPPPPISNGDTTSFLLKVYVPQSRVEYQC |
| NOV2b | ESSYGNTTDSIVCGEDGWSHLPTCYNSSESCGPPPPISNGDTTSFPQKVYLPWSRVEYQC |
| NOV2a | QSYYELQGSNYVTCSNGEWSEPPRCIRIHFCR---------------------------- |
| NOV2b | QSYYELQGSKYVTCSNGDWSEPPRCISMKPCEFPEIQHGHLYYENTRRPYFPVATGQSYS |
| NOV2a | ------------------------------------------------------------ |
| NOV2b | YYCDQNFVTPSGSYWDYIHCTQDGWLPTVPCLRTCSKSDIEIENGFISESSSIYILNKEI |
| NOV2a | ------------------------------------------------------------ |
| NOV2b | QYKCKPGYATADGNSSGSITCLQNGWSAQPICIKFCDMPVFENSRAKSNGMRFKLHDTLD |
| NOV2a | ------------------------------------------------------------ |
| NOV2b | YECYDGYEISYGNTTGSIVCGEDGWSHFPTCYNSSEKCGPPPPISNGDTTSFLLKVYVPQ |
| NOV2a | ------------------------------------------------------------ |
| NOV2b | SRVEYQCQSYYELQGSNYVTCSNGEWSEPPRCIHPCIITEENMNKNNIQLKGKSDIKYYA |
| NOV2a | -------------------------------------- |
| NOV2b | KTGDTIEFMCKLGYNANTSVLSFQAVCREGIVEYPRCE |
| NOV2a | (SEQ ID NO: 12) |
| NOV2b | (SEQ ID NO: 14) |

Further analysis of the NOV2a protein yielded the following properties shown in Table 2C.

TABLE 2C
Protein Sequence Properties NOV2a

| SignalP | Cleavage site between residues 19 and 20 | | | |
|---|---|---|---|---|
| analysis: | | | | |
| PSORT II | PSG: a new signal peptide prediction method | | | |
| analysis: | N-region: length 0; pos. chg 0; neg. chg 0 | | | |
| | H-region: length 20; peak value 9.20 | | | |
| | PSG score: 4.80 | | | |
| | GvH: von Heijne's method for signal seq. recognition | | | |
| | GvH score (threshold: −2.1): 1.38 | | | |
| | possible cleavage site: between 18 and 19 | | | |
| | >>> Seems to have a cleavable signal peptide (1 to 18) | | | |
| | ALOM: Klein et al's method for TM region allocation | | | |
| | Init position for calculation: 19 | | | |
| | Tentative number of TMS(s) for the threshold 0.5: 0 | | | |
| | number of TMS(s) . . . fixed | | | |
| | PERIPHERAL Likelihood = 8.27 (at 137) | | | |
| | ALOM score: 8.27 (number of TMSs: 0) | | | |
| | MTOP: Prediction of membrane topology (Hartmann et al.) | | | |
| | Center position for calculation: 9 | | | |
| | Charge difference: 0.0 C(1.0)-N(1.0) | | | |
| | N >= C: N-terminal side will be inside | | | |
| | MITDISC: discrimination of mitochondrial targeting seq | | | |
| | R content: | 0 | Hyd Moment (75): | 3.43 |
| | Hyd Moment(95): | 4.91 | G content: | 1 |
| | D/E content: | 1 | S/T content: | 2 |

Score: −5.38
Gavel: prediction of cleavage sites for mitochondrial preseq
cleavage site motif not found
NUCDISC: discrimination of nuclear localization signals
pat4: none
pat7: none
bipartite: none
content of basic residues: 7.4%
NLS Score: −0.47
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: nuclear
Reliability: 76.7
COIL: Lupas's algorithm to detect coiled-coil regions
total: 0 residues
Final Results (k = 9/23):
44.4%: extracellular, including cell wall
33.3%: nuclear
22.2%: mitochondrial
>> prediction for CG106298-02 is exc (k = 9)

A search of the NOV2a protein against the Geneseq database, a proprietary database that contains sequences published in patents and patent publication, yielded several homologous proteins shown in Table 2D.

TABLE 2D
Geneseq Results for NOV2a

| | | NOV2a | Identities/ | |
|---|---|---|---|---|
| | | Residues/ | Similarities | |
| Geneseq | Protein/Organism/Length | Match | for the | Expect |
| Identifier | [Patent#, Date] | Residues | Matched Region | Value |
| AAY09065 | Human complement factor H | 12 . . . 265 | 241/263 (91%) | e−152 |
| | homolog protein - Homo sapiens, 578 | 251 . . . 513 | 250/263 (94%) | |
| | aa. [WO9918200-A1, 15 APR. 1999] | | | |
| ABU07436 | Protein differentially regulated in | 10 . . . 253 | 135/303 (44%) | 9e−68 |
| | prostate cancer #39 - Homo sapiens, | 312 . . . 611 | 171/303 (55%) | |
| | 1231 aa. [WO200281638-A2, 17 | | | |
| | OCT. 2002] | | | |
| AAB43738 | Human cancer associated protein | 1 . . . 265 | 108/270 (40%) | 2e−56 |
| | sequence SEQ ID NO: 1183 - Homo | 13 . . . 275 | 154/270 (57%) | |
| | sapiens, 342 aa. [WO200055350-A1, | | | |
| | 21 SEP. 2000] | | | |
| ABB80571 | Human sbg614126complfH protein | 1 . . . 265 | 113/269 (42%) | 2e−56 |
| | #2 - Homo sapiens, 327 aa. | 1 . . . 262 | 154/269 (57%) | |
| | [WO200222802-A1, 21 MAR. 2002] | | | |
| ABB80570 | Human sbg614126complfH protein | 23 . . . 265 | 102/247 (41%) | 5e−51 |
| | #1 - Homo sapiens, 364 aa. | 60 . . . 299 | 141/247 (56%) | |
| | [WO200222802-A1, 21 MAR. 2002] | | | |

In a BLAST search of public sequence databases, the NOV2a protein was found to have homology to the proteins shown in the BLASTP data in Table 2E.

TABLE 2E
Public BLASTP Results for NOV2a

| | | NOV2a | Identities/ | |
|---|---|---|---|---|
| Protein | | Residues/ | Similarities | |
| Accession | | Match | for the | Expect |
| Number | Protein/Organism/Length | Residues | Matched Portion | Value |
| Q92496 | Complement factor H-related protein | 1 . . . 265 | 255/266 (95%) | e−160 |
| | 4 precursor (FHR-4) - Homo sapiens | 1 . . . 266 | 262/266 (97%) | |
| | (Human), 331 aa. | | | |
| Q02985 | Complement factor H-related protein | 1 . . . 265 | 172/270 (63%) | e−101 |
| | 3 precursor (FHR-3) (H factor-like | 1 . . . 265 | 198/270 (72%) | |
| | protein 3) (DOWN16) - Homo | | | |
| | sapiens (Human), 330 aa. | | | |
| A45222 | complement factor H-related protein | 1 . . . 265 | 173/269 (64%) | e−101 |
| | DOWN16 precursor - human, 331 aa. | 1 . . . 266 | 196/269 (72%) | |
| Q8R018 | Hypothetical 58.1 kDa protein - Mus | 10 . . . 270 | 150/320 (46%) | 6e−81 |
| | musculus (Mouse), 509 aa. | 130 . . . 447 | 181/320 (55%) | |
| Q61407 | Complement factor H-related protein - | 10 . . . 270 | 150/320 (46%) | 6e−81 |
| | Mus musculus (Mouse), 452 aa | 73 . . . 390 | 181/320 (55%) | |
| | (fragment). | | | |

PFam analysis predicts that the NOV2a protein contains the domains shown in the Table 2F.

TABLE 2F
Domain Analysis of NOV2a

| | | Identities/ | |
|---|---|---|---|
| | NOV2a | Similarities | |
| Pfam | Match | for the | Expect |
| Domain | Region | Matched Region | Value |
| Sushi | 23 . . . 83 | 17/69 (25%) | 1.2e−08 |
| | | 47/69 (68%) | |
| Sushi | 87 . . . 144 | 22/68 (32%) | 2.2e−12 |
| | | 45/68 (66%) | |
| Sushi | 148 . . . 203 | 22/66 (33%) | 3.9e−08 |
| | | 44/66 (67%) | |
| Sushi | 210 . . . 264 | 23/65 (35%) | 5.4e−15 |
| | | 42/65 (65%) | |

### Example 3

The NOV3 clone was analyzed, and the nucleotide and encoded polypeptide sequences are shown in Table 3A.

TABLE 3A
NOV3 Sequence Analysis

NOV3a,
CG110590-02 SEQ ID NO: 15 1487 bp
DNA Sequence ORF Start: ATG at 112 ORF Stop: TGA at 1303
GGAGAAGGCCAGTGCCCAGGTTAGTGAGCAGTGCCCGGCGCCCGCTTCCCTCACCTCCTTTTCCAGCC
TTTGCACAGCTTGAAGGTTCTGTCACCTTTTGCAGTGGTCCAAATGAGAAAAAAGTGGAAAATGGGAG
GCATGAAATACATCTTTTCGTTGTTGTTCTTTCTTTTGCTAGAAGGAGGCAAAACAGAGCAAGTAAAA
CATTCAGAGACATATTGCATGTTTCAAGACAAGAAGTACAGAGTGGGTGAGAGATGGCATCCTTACCT
GGAACCTTATGGGTTGGTTTACTGCGTGAACTGCATCTGCTCAGAGAATGGGAATGTGCTTTGCAGCC
GAGTCAGATGTCCAAATGTTCATTGCCTTTCTCCTGTGCATATTCCTCATCTGTGCTGCCCTCGCTGC
CCAGACTCCTTACCCCCAGTGAACAATAAGGTGACCAGCAAGTCTTGCGAGTACAATGGGACAACTTA
CCAACATGGAGAGCTGTTCGTAGCTGAAGGGCTCTTTCAGAATCGGCAACCCAATCAATGCACCCAGT
GCAGCTGTTCGGAGGGAAACGTGTATTGTGGTCTCAAGACTTGCCCCAAATTAACCTGTGCCTTCCCA
GTCTCTGTTCCAGATTCCTGCTGCCGGGTATGCAGAGGAGATGGAGAACTGTCATGGGAACATTCTGA
TGGTGATATCTTCCGGCAACCTGCCAACAGAGAAGCAAGACATTCTTACCACCGCTCTCACTATGATC
CTCCACCAAGCCGACAGGCTGGAGGTCTGTCCCGCTTTCCTGGGGCCAGAAGTCACCGGGGAGCTCTT
ATGGATTCCCAGCAAGCATCAGGAACCATTGTGCAAATTGTCATCAATAACAAACACAAGCATGGACA
AGTGTGTGTTTCCAATGGAAAGACCTATTCTCATGGCGAGTCCTGGCACCCAAACCTCCGGGCATTTG
GCATTGTGGAGTGTGTGCTATGTACTTGTAATGTCACCAAGCAAGAGTGTAAGAAAATCCACTGCCCC
AATCGATACCCCTGCAAGTATCCTCAAAAAATAGACGGAAAATGCTGCAAGGTGTGTCCAGGTAAAAA
AGCAAAAGAACTTCCAGGCCAAAGCTTTGACAATAAAGGATACTTCTGCGGGGAAGAAACGATGCCTG
TGTATGAGTCTGTATTCATGGAGGATGGGGAGACAACCAGAAAAATAGCACTGGAGACTGAGAGACCA
CCTCAGGCATTCTCCAGCACTTCCATATTGAGAAGATCTCCAAGAGGATGTTTGAGGAGCTTCCTCAC
TTCAAGCTGGTGACCAGAACAACCCTGAGCCAGTGGAAGATCTTCACCGAAGGAGAAGCTCAGATCAG
CCAGATGTGTTCAAGTCGTGTATGCAGAACAGAGCTTGAAGATTTAGTCAAGGTTTTGTACCTGGAGA
GATCTGAAAAGGGCCACTGTTAGGCAAGACAGACAGTATTGGATAGGGTAAAGCAAGAA
NOV3a,
CG110590-02
Protein Sequence SEQ ID NO: 16 397 aa MW at 44841.9 kD
MRKKWKMGGMKYIFSLLFFLLLEGGKTEQVKHSETYCMFQDKKYRVGERWHPYLEPYGLVYCVNCICS
ENGNVLCSRVRCPNVHCLSPVHIPHLCCPRCPDSLPPVNNKVTSKSCEYNGTTYQHGELFVAEGLFQN
RQPNQCTQCSCSEGNVYCGLKTCPKLTCAFPVSVPDSCCRVCRGDGELSWEHSDGDIFRQPANREARH
SYHRSHYDPPPSRQAGGLSRFPGARSHRGALMDSQQASGTIVQIVINNKHKHGQVCVSNGKTYSHGES
WHPNLRAFGIVECVLCTCNVTKQECKKIHCPNRYPCKYPQKIDGKCCKVCPGKKAKELPGQSFDNKGY
FCGEETMPVYESVFMEDGETTRKIALETERPPQAFSSTSILRRSPRGCLRSFLTSSW
NOV3b,
CG110590-01 SEQ ID NO: 17 1440 bp
DNA Sequence ORF Start: ATG at 18 ORF Stop: TAG at 1374
TGAGAAAAAAGTGGAAAATGGGAGGCATGAAATACATCTTTTCGTTGTTGTTCTTTCTTTTGCTAGAA
GGAGGCAAAACAGAGCAAGTAAAACATTCAGAGACATATTGCATGTTTCAAGACAAGAAGTACAGAGT
GGGTGAGAGATGGCATCCTTACCTGGAACCTTATGGGTTGGTTTACTGCGTGAACTGCATCTGCTCAG
AGAATGGGAATGTGCTTTGCAGCCGAGTCAGATGTCCAAATGTTCATTGCCTTTCTCCTGTGCATATT
CCTCATCTGTGCTGCCCTCGCTGCCCAGAAGACTCCTTACCCCCAGTGAACAATAAGGTGACCAGCAA
GTCTTGCGAGTACAATGGGACAACTTACCAACATGGAGAGCTGTTCGTAGCTGAAGGGCTCTTTCAGA
ATCGGCAACCCAATCAATGCACCCAGTGCAGCTGTTCGGAGGGAAACGTGTATTGTGGTCTCAAGACT
TGCCCCAAATTAACCTGTGCCTTCCCAGTCTCTGTTCCAGATTCCTGCTGCCGGGTATGCAGAGGAGA
TGGAGAACTGTCATGGGAACATTCTGATGGTGATATCTTCCGGCAACCTGCCAACAGAGAAGCAAGAC
ATTCTTACCACCGCTCTCACTATGATCCTCCACCAAGCCGACAGGCTGGAGGTCTGTCCCGCTTTCCT
GGGGCCAGAAGTCACCGGGGAGCTCTTATGGATTCCCAGCAAGCATCAGGAACCATTGTGCAAATTGT
CATCAATAACAAACACAAGCATGGACAAGTGTGTGTTTCCAATGGAAAGACCTATTCTCATGGCGAGT
CCTGGCACCCAAACCTCCGGGCATTTGGCATTGTGGAGTGTGTGCTATGTACTTGTAATGTCACCAAG
CAAGAGTGTAAGAAAATCCACTGCCCCAATCGATACCCCTGCAAGTATCCTCAAAAAATAGACGGAAA
ATGCTGCAAGGTGTGTCCAGGTAAAAAAGCAAAAGAAGAACTTCCAGGCCAAAGCTTTGACAATAAAG
GCTACTTCTGCGGGGAAGAAACGATGCCTGTGTATGAGTCTGTATTCATGGAGGATGGGGAGACAACC
AGAAAAATAGCACTGGAGACTGAGAGACCACCTCAGGTAGAGGTCCACGTTTGGACTATTCGAAAGGG
CATTCTCCAGCACTTCCATATTGAGAAGATCTCCAAGAGGATGTTTGAGGAGCTTCCTCACTTCAAGC
TGGTGACCAGAACAACCCTGAGCCAGTGGAAGATCTTCACCGAAGGAGAAGCTCAGATCAGCCAGATG
TGTTCAAGTCGTGTATGCAGAACAGAGCTTGAAGATTTAGTCAAGGTTTTGTACCTGGAGAGATCTGA
AAAGGGCCACTGTTAGGCAAGACAGACAGTATTGGATAGGGTAAAGCAAGAAAACTCAAGCTGCAGCT
GGACTGCAGGCT
NOV3b,
CG110590-01
Protein Sequence SEQ ID NO: 18 452 aa MW at 51425.5 kD
MGGMKYI FSLLFFLLLEGGKTEQVKHSETYCMFQDKKYRVGERWHPYLEPYGLVYCVNCICSENGNVL
CSRVRCPNVHCLSPVHIPHLCCPRCPEDSLPPVNNKVTSKSCEYNGTTYQHGELFVAEGLFQNRQPNQ
CTQCSCSEGNVYCGLKTCPKLTCAFPVSVPDSCCRVCRGDGELSWEHSDGDIFRQPANREARHSYHRS
HYDPPPSRQAGGLSRFPGARSHRGALMDSQQASGTIVQIVINNKHKHGQVCVSNGKTYSHGESWHPNL
RAFGIVECVLCTCNVTKQECKKIHCPNRYPCKYPQKIDGKCCKVCPGKKAKEELPGQSFDNKGYFCGE
ETMPVYESVFMEDGETTRKIALETERPPQVEVHVWTIRKGILQHFHIEKISKRMFEELPHFKLVTRTT
LSQWKIFTEGEAQISQMCSSRVCRTELEDLVKVLYLERSEKGHC
NOV3c, 13382325 1487 bp, SNP: T/C
SNP CG110590-02 SEQ ID NO: 19 at position 454
DNA Sequence ORF Start: ATG at 112 ORF Stop: 1303
GGAGAAGGCCAGTGCCCAGGTTAGTGAGCAGTGCCCGGCGCCCGCTTCCCTCACCTCCTTTTCCAGCCTTT
GCACAGCTTGAAGGTTCTGTCACCTTTTGCAGTGGTCCAAATGAGAAAAAAGTGGAAAATGGGAGGCATGA
ACATATTGCATGTTTCAAGACAAGAAGTACAGAGTGGGTGAGAGATGGCATCCTTACCTGGAACCTTATGG
ACATATTGCATGTTTCAAGACAAGAAGTACAGAGTGGGTGAGAGATGGCATCCTTACCTGGAACCTTATGG
GTTGGTTTACTGCGTGAACTGCATCTGCTCAGAGAATGGGAATGTGCTTTGCAGCCGAGTCAGATGTCCAA
ATGTTCATTGCCTTTCTCCTGTGCATATTCCTCATCTGTGCTGCCCTCGCTGCCCAGACTCCTTACCCCCA
GTGAACAATAAGGTGACCAGCAAGTCTCGCGAGTACAATGGGACAACTTACCAACATGGAGAGCTGTTCGT
AGCTGAAGGGCTCTTTCAGAATCGGCAACCCAATCAATGCACCCAGTGCAGCTGTTCGGAGGGAAACGTGT
ATTGTGGTCTCAAGACTTGCCCCAAATTAACCTGTGCCTTCCCAGTCTCTGTTCCAGATTCCTGCTGCCGG
GTATGCAGAGGAGATGGAGAACTGTCATGGGAACATTCTGATGGTGATATCTTCCGGCAACCTGCCAACAG
AGAAGCAAGACATTCTTACCACCGCTCTCACTATGATCCTCCACCAAGCCGACAGGCTGGAGGTCTGTCCC
GCTTTCCTGGGGCCAGAAGTCACCGGGGAGCTCTTATGGATTCCCAGCAAGCATCAGGAACCATTGTGCAA
ATTGTCATCAATAACAAACACAAGCATGGACAAGTGTGTGTTTCCAATGGAAAGACCTATTCTCATGGCGA
GTCCTGGCACCCAAACCTCCGGGCATTTGGCATTGTGGAGTGTGTGCTATGTACTTGTAATGTCACCAAGC
AAGAGTGTAAGAAAATCCACTGCCCCAATCGATACCCCTGCAAGTATCCTCAAAAAATAGACGGAAAATGC
TGCAAGGTGTGTCCAGGTAAAAAAGCAAAAGAACTTCCAGGCCAAAGCTTTGACAATAAAGGATACTTCTG
CGGGGAAGAAACGATGCCTGTGTATGAGTCTGTATTCATGGAGGATGGGGAGACAACCAGAAAAATAGCAC
TGGAGACTGAGAGACCACCTCAGGCATTCTCCAGCACTTCCATATTGAGAAGATCTCCAAGAGGATGTTTG
AGGAGCTTCCTCACTTCAAGCTGGTGACCAGAACAACCCTGAGCCAGTGGAAGATCTTCACCGAAGGAGAA
GCTCAGATCAGCCAGATGTGTTCAAGTCGTGTATGCAGAACAGAGCTTGAAGATTTAGTCAAGGTTTTGTA
CCTGGAGAGATCTGAAAAGGGCCACTGTTAGGCAAGACAGACAGTATTGGATAGGGTAAAGCAAGAA
NOV3c, 13382325
SNP CG110590-02
Protein Sequence SEQ ID NO: 20 397 aa SNP: Cys to Arg at 115
MRKKWKMGGMKYIFSLLFFLLLEGGKTEQVKHSETYCMFQDKKYRVGERWHPYLEPYGLVYCVNCICSENG
NVLCSRVRCPNVHCLSPVHIPHLCCPRCPDSLPPVNNKVTSKSREYNGTTYQHGELFVAEGLFQNRQPNQC
TQCSCSEGNVYCGLKTCPKLTCAFPVSVPDSCCRVCRGDGELSWEHSDGDIFRQPANREARHSYHRSHYDP
PPSRQAGGLSRFPGARSHRGALMDSQQASGTIVQIVINNKHKHGQVCVSNGKTYSHGESWHPNLRAFGIVE
CVLCTCNVTKQECKKIHCPNRYPCKYPQKIDGKCCKVCPGKKAKELPGQSFDNKGYFCGEETMPVYESVFM
EDGETTRKIALETERPPQAFSSTSILRRSPRGCLRSFLTSSW
NOV3d, 13382326
SNP CG110590-02 SEQ ID NO: 21 1440 bp SNP: A/G at 756
DNA Sequence ORF Start: ATG at 112 ORF Stop: end of sequence
GGAGAAGGCCAGTGCCCAGGTTAGTGAGCAGTGCCCGGCGCCCGCTTCCCTCACCTCCTTTTCCAGCCTTT
GCACAGCTTGAAGGTTCTGTCACCTTTTGCAGTGGTCCAAATGAGAAAAAAGTGGAAAATGGGAGGCATGA
AATACATCTTTTCGTTGTTGTTCTTTCTTTTGCTAGAAGGAGGCAAAACAGAGCAAGTAAAACATTCAGAG
ACATATTGCATGTTTCAAGACAAGAAGTACAGAGTGGGTGAGAGATGGCATCCTTACCTGGAACCTTATGG
GTTGGTTTACTGCGTGAACTGCATCTGCTCAGAGAATGGGAATGTGCTTTGCAGCCGAGTCAGATGTCCAA
ATGTTCATTGCCTTTCTCCTGTGCATATTCCTCATCTGTGCTGCCCTCGCTGCCCAGACTCCTTACCCCCA
GTGAACAATAAGGTGACCAGCAAGTCTTGCGAGTACAATGGGACAACTTACCAACATGGAGAGCTGTTCGT
AGCTGAAGGGCTCTTTCAGAATCGGCAACCCAATCAATGCACCCAGTGCAGCTGTTCGGAGGGAAACGTGT
ATTGTGGTCTCAAGACTTGCCCCAAATTAACCTGTGCCTTCCCAGTCTCTGTTCCAGATTCCTGCTGCCGG
GTATGCAGAGGAGATGGAGAACTGTCATGGGAACATTCTGATGGTGATATCTTCCGGCAACCTGCCAACAG
AGAAGCAAGACATTCTTACCACCGCTCTCACTATGATCCTCCACCGAGCCGACAGGCTGGAGGTCTGTCCC
GCTTTCCTGGGGCCAGAAGTCACCGGGGAGCTCTTATGGATTCCCAGCAAGCATCAGGAACCATTGTGCAA
ATTGTCATCAATAACAAACACAAGCATGGACAAGTGTGTGTTTCCAATGGAAAGACCTATTCTCATGGCGA
GTCCTGGCACCCAAACCTCCGGGCATTTGGCATTGTGGAGTGTGTGCTATGTACTTGTAATGTCACCAAGC
AAGAGTGTAAGAAAATCCACTGCCCCAATCGATACCCCTGCAAGTATCCTCAAAAAATAGACGGAAAATGC
TGCAAGGTGTGTCCAGGTAAAAAAGCAAAAGAACTTCCAGGCCAAAGCTTTGACAATAAAGGATACTTCTG
CGGGGAAGAAACGATGCCTGTGTATGAGTCTGTATTCATGGAGGATGGGGAGACAACCAGAAAAATAGCAC
TGGAGACTGAGAGACCACCTCAGGCATTCTCCAGCACTTCCATATTGAGAAGATCTCCAAGAGGATGTTTG
AGGAGCTTCCTCACTTCAAGCTGGTGACCAGAACAACCCTGAGCCAGTGGAAGATCTTCACCGAAGGAGAA
GCTCAGATCAGCCAGATGTGTTCAAGTCGTGTATGCAGAACAGAGCTTGAAGATTTAGTCAAGGTTTTGTA
CCTGGAGAGATCTGAAAAGGGCCACTGTTAGGCAAGACAGACAGTATTGGATAGGGTAAAGCAAGAA
NOV3d, 13382326
SNP CG110590-02 SNP: No change in protein
Protein Sequence SEQ ID NO: 22 397 aa sequence
MRKKWKMGGMKYIFSLLFFLLLEGGKTEQVKHSETYCMFQDKKYRVGERWHPYLEPYGLVYCVNCICSENG
NVLCSRVRCPNVHCLSPVHIPHLCCPRCPDSLPPVNNKVTSKSCEYNGTTYQHGELFVAEGLFQNRQPNQC
TQCSCSEGNVYCGLKTCPKLTCAFPVSVPDSCCRVCRGDGELSWEHSDGDIFRQPANREARHSYHRSHYDP
PPSRQAGGLSRFPGARSHRGALMDSQQASGTIVQIVINNKHKHGQVCVSNGKTYSHGESWHPNLRAFGIVE
CVLCTCNVTKQECKKIHCPNRYPCKYPQKIDGKCCKVCPGKKAKELPGQSFDNKGYFCGEETMPVYESVFM
EDGETTRKIALETERPPQAFSSTSILRRSPRGCLRSFLTSSW

A ClustalW comparison of the above protein sequences yields the following sequence alignment shown in Table 3B.

TABLE 3B
Comparison of the NOV3 protein sequences.

| | |
|---|---|
| NOV3a | MRKKWKMGGMKYIFSLLFFLLLEGGKTEQVKHSETYCMFQDKKYRVGERWHPYLEPYGLV |
| NOV3b | ------MGGMKYIFSLLFFLLLEGGKTEQVKHSETYCMFQDKKYRVGERWHPYLEPYGLV |
| NOV3a | YCVNCICSENGNVLCSRVRCPNVHCLSPVHIPHLCCPRCP-DSLPPVNNKVTSKSCEYNG |
| NOV3b | YCVNCICSENGNVLCSRVRCPNVHCLSPVHIPHLCCPRCPEDSLPPVNNKVTSKSCEYNG |
| NOV3a | TTYQHGELFVAEGLFQNRQPNQCTQCSCSEGNVYCGLKTCPKLTCAFPVSVPDSCCRVCR |
| NOV3b | TTYQHGELFVAEGLFQNRQPNQCTQCSCSEGNVYCGLKTCPKLTCAFPVSVPDSCCRVCR |
| NOV3a | GDGELSWEHSDGDIFRQPANREARHSYHRSHYDPPPSRQAGGLSRFPGARSHRGALMDSQ |
| NOV3b | GDGELSWEHSDGDIFRQPANREARHSYHRSHYDPPPSRQAGGLSRFPGARSHRGALMDSQ |
| NOV3a | QASGTIVQIVINNKHKHGQVCVSNGKTYSHGESWHPNLRAFGIVECVLCTCNVTKQECKK |
| NOV3b | QASGTIVQIVINNKHKHGQVCVSNGKTYSHGESWHPNLRAFGIVECVLCTCNVTKQECKK |
| NOV3a | IHCPNRYPCKYPQKIDGKCCKVCPGKKAK-ELPGQSFDNKGYFCGEETMPVYESVFMEDG |
| NOV3b | IHCPNRYPCKYPQKIDGKCCKVCPGKKAKEELPGQSFDNKGYFCGEETMPVYESVFMEDG |
| NOV3a | ETTRKIALETERPP--------------QAFSSTSILRRS----PRGCLRS-FLTSSW-- |
| NOV3b | ETTRKIALETERPPQVEVHVWTIRKGILQHFHIEKISKRMFEELPHFKLVTRTTLSQWKI |
| NOV3a | -------------------------------------- |
| NOV3b | FTEGEAQISQMCSSRVCRTELEDLVKVLYLERSEKGHC |
| NOV3a | (SEQ ID NO: 16) |
| NOV3b | (SEQ ID NO: 18) |

Further analysis of the NOV3a protein yielded the following properties shown in Table 3C.

TABLE 3C
Protein Sequence Properties NOV3a

| SignalP | Cleavage site between residues 28 and 29 | | | |
|---|---|---|---|---|
| analysis: | | | | |
| PSORT II | PSG: a new signal peptide prediction method | | | |
| analysis: | N-region: length 11; pos. chg 5; neg. chg 0 | | | |
| | H-region: length 11; peak value 12.14 | | | |
| | PSG score: 7.74 | | | |
| | GvH: von Heijne's method for signal seq. recognition | | | |
| | GvH score (threshold: −2.1): −1.64 | | | |
| | possible cleavage site: between 27 and 28 | | | |
| | >>> Seems to have a cleavable signal peptide (1 to 27) | | | |
| | ALOM: Klein et al's method for TM region allocation | | | |
| | Init position for calculation: 28 | | | |
| | Tentative number of TMS(s) for the threshold 0.5: 0 | | | |
| | number of TMS(s) . . . fixed | | | |
| | PERIPHERAL Likelihood = 1.80 (at 277) | | | |
| | ALOM score: 1.80 (number of TMSs: 0) | | | |
| | MTOP: Prediction of membrane topology (Hartmann et al.) | | | |
| | Center position for calculation: 13 | | | |
| | Charge difference: −6.5 C(−0.5)-N(6.0) | | | |
| | N >= C: N-terminal side will be inside | | | |
| | MITDISC: discrimination of mitochondrial targeting seq | | | |
| | R content: | 1 | Hyd Moment (75): | 6.12 |
| | Hyd Moment(95): | 9.66 | G content: | 2 |
| | D/E content: | 1 | S/T content: | 1 |

Score: −4.32
Gavel: prediction of cleavage sites for mitochondrial preseq
R-2 motif at 12 MRK|KW
NUCDISC: discrimination of nuclear localization signals
pat4: none
pat7: PGKKAKE (4) at 323
bipartite: none
content of basic residues: 13.1%
NLS Score: −0.13
KDEL: ER retention motif in the C-terminus: none
ER Membrane Retention Signals:
XXRR-like motif in the N-terminus: RKKW
none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: nuclear
Reliability: 94.1
COIL: Lupas's algorithm to detect coiled-coil regions
total: 0 residues
Final Results (k = 9/23):
66.7%: extracellular, including cell wall
11.1%: mitochondrial
11.1%: vacuolar
11.1%: nuclear
>> prediction for CG110590-02 is exc (k = 9)

A search of the NOV3a protein against the Geneseq database, a proprietary database that contains sequences published in patents and patent publication, yielded several homologous proteins shown in Table 3D.

TABLE 3D
Geneseq Results for NOV3a

| | | NOV3a | Identities/ | |
|---|---|---|---|---|
| | | Residues/ | Similarities for | |
| Geneseq | Protein/Organism/Length [Patent | Match | the Matched | Expect |
| Identifier | #, Date] | Residues | Region | Value |
| AAY53035 | Human secreted protein clone | 1 . . . 373 | 373/374 (99%) | 0.0 |
| | dw665_4 protein sequence SEQ ID | 1 . . . 374 | 373/374 (99%) | |
| | NO: 76 - Homo sapiens, 457 aa. | | | |
| | [WO9957132-A1, 11 NOV. 1999] | | | |
| AAY82777 | Human chordin related protein (Clone | 1 . . . 373 | 373/374 (99%) | 0.0 |
| | dw665_4) - Homo sapiens, 457 aa. | 1 . . . 374 | 373/374 (99%) | |
| | [WO200009551-A1, 24 FEB. 2000] | | | |
| AAM39408 | Human polypeptide SEQ ID NO 2553 - | 1 . . . 373 | 371/375 (98%) | 0.0 |
| | Homo sapiens, 458 aa. | 1 . . . 375 | 372/375 (98%) | |
| | [WO200153312-A1, 26 JUL. 2001] | | | |
| AAB65027 | Gene #1 associated peptide #2 - | 1 . . . 373 | 368/374 (98%) | 0.0 |
| | Homo sapiens, 489 aa. | 37 . . . 406 | 369/374 (98%) | |
| | [WO200075375-A1, 14 DEC. 2000] | | | |
| AAB64993 | Human secreted protein #1 - Homo | 1 . . . 373 | 368/374 (98%) | 0.0 |
| | sapiens, 453 aa. [WO200075375-A1, | 1 . . . 370 | 369/374 (98%) | |
| | 14 DEC. 2000] | | | |

In a BLAST search of public sequence databases, the NOV3a protein was found to have homology to the proteins shown in the BLASTP data in Table 3E.

TABLE 3E
Public BLASTP Results for NOV3a

| | | NOV3a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9BU40 | Neuralin precursor (Ventroptin) - | 7 . . . 373 | 367/367 (100%) | 0.0 |
| | Homo sapiens (Human), 450 aa. | 1 . . . 367 | 367/367 (100%) | |
| CAC43868 | Sequence 7 from Patent | 7 . . . 373 | 367/369 (99%) | 0.0 |
| | WO0142465 precursor - Homo | 1 . . . 369 | 367/369 (99%) | |
| | sapiens (Human), 452 aa. | | | |
| CAC43869 | Sequence 11 from Patent | 7 . . . 373 | 362/368 (98%) | 0.0 |
| | WO0142465 precursor - Homo | 1 . . . 364 | 363/368 (98%) | |
| | sapiens (Human), 447 aa | | | |
| | (fragment). | | | |
| Q920C1 | Neuralin precursor (Ventroptin) - | 7 . . . 373 | 334/368 (90%) | 0.0 |
| | Mus musculus (Mouse), 447 aa. | 1 . . . 364 | 351/368 (94%) | |
| CAC43867 | Sequence 4 from Patent | 7 . . . 377 | 327/372 (87%) | 0.0 |
| | WO0142465 precursor - Rattus | 1 . . . 368 | 346/372 (92%) | |
| | norvegicus (Rat), 382 aa. | | | |

PFam analysis predicts that the NOV3a protein contains the domains shown in the Table 3F.

TABLE 3F
Domain Analysis of NOV3a

| | | Identities/ | |
|---|---|---|---|
| | NOV3a | Similarities | |
| Pfam | Match | for the | Expect |
| Domain | Region | Matched Region | Value |
| Vwc | 37 . . . 99 | 25/84 (30%) | 1.5e−10 |
| | | 39/84 (46%) | |
| Vwc | 115 . . . 178 | 26/90 (29%) | 8e−09 |
| | | 48/90 (53%) | |
| Vwc | 260 . . . 322 | 27/84 (32%) | 1.5e−11 |
| | | 41/84 (49%) | |

### Example 4

The NOV4 clone was analyzed, and the nucleotide and encoded polypeptide sequences are shown in Table 4A.

TABLE 4A
NOV4 Sequence Analysis

NOV4a,
CG114555-01 SEQ ID NO: 23 1710 bp
DNA Sequence ORF Start: ATG at 14 ORF Stop: TAA at 1534
GTCACTGAGACCCATGGCAAGGAAACAAAATAGGAATTCCAAGGAACTGGGCCTAGTTCCCCTCACAG
ATGACACCAGCCACGCCGGGCCTCCAGGGCCAGGGAGGGCACTGCTGGAGTGTGACCACCTGAGGAGT
GGGGTGCCAGGTGGAAGGAGAAGAAAGTACATCAAGGCCTTTTACAATGAGTCATGGGAAAGAAGGCA
TGGACGTCCAATAGACCCAGACACTCTGACTCTGCTCTGGTCTGTGACTGTGTCCATATTCGCCATCG
GTGGACTTGTGGGGACATTAATTGTGAAGATGATTGGAAAGGTTCTTGGGAGGAAGCACACTTTGCTG
GCCAATAATGGGTTTGCAATTTCTGCTGCATTGCTGATGGCCTGCTCGCTCCAGGCAGGAGCCTTTGA
AATGCTCATCGTGGGACGCTTCATCATGGGCATAGATGGAGGCGTCGCCCTCAGTGTGCTCCCCATGT
ACCTCAGTGAGATCTCACCCAAGGAGATCCGTGGCTCTCTGGGGCAGGTGACTGCCATCTTTATCTGC
ATTGGCGTGTTCACTGGGCAGCTTCTGGGCCTGCCCGAGCTGCTGGGAAAGGAGAGTACCTGGCCATA
CCTGTTTGGAGTGATTGTGGTCCCTGCCGTTGTCCAGCTGCTGAGCCTTCCCTTTCTCCCGGACAGCC
CACGCTACCTGCTCTTGGAGAAGCACAACGAGGCAAGAGCTGTGAAAGCCTTCCAAACGTTCTTGGGT
AAAGCAGACGTTTCCCAAGAGGTAGAGGAGGTCCTGGCTGAGAGCCGCGTGCAGAGGAGCATCCGCCT
GGTGTCCGTGCTGGAGCTGCTGAGAGCTCCCTACGTCCGCTGGCAGGTGGTCACCGTGATTGTCACCA
TGGCCTGCTACCAGCTCTGTGGCCTCAATGCAATTTGGTTCTATACCAACAGCATCTTTGGAAAAGCT
GGGATCCCTCTGGCAAAGATCCCATACGTCACCTTGAGTACAGGGGGCATCGAGACTTTGGCTGCCGT
CTTCTCTGGTTTGGTCATTGAGCACCTGGGACGGAGACCCCTCCTCATTGGTGGCTTTGGGCTCATGG
GCCTCTTCTTTGGGACCCTCACCATCACGCTGACCCTGCAGGACCACGCCCCCTGGGTCCCCTACCTG
AGTATCGTGGGCATTCTGGCCATCATCGCCTCTTTCTGCAGTGGGCCAGGTGGCATCCCGTTCATCTT
GACTGGTGAGTTCTTCCAGCAATCTCAGCGGCCGGCTGCCTTCATCATTGCAGGCACCGTCAACTGGC
TCTCCAACTTTGCTGTTGGGCTCCTCTTCCCATTCATTCAGAAAAGTCTGGACACCTACTGTTTCCTA
GTCTTTGCTACAATTTGTATCACAGGTGCTATCTACCTGTATTTTGTGCTGCCTGAGACCAAAAACAG
AACCTATGCAGAAATCAGCCAGGCATTTTCCAAAAGGAACAAAGCATACCCACCAGAAGAGAAAATCG
ACTCAGCTGTCACTGATGGTAAGATAAATGGAAGGCCTTAACAAGTTTCCTCCTCCACGTTGGACAAT
TATGTCAAAAACAGGATTGTCTACATGGATGATCTCACTTTTCAGGAAACTTAAAATTTACCCATTAT
TGGGAAGCTTAAATGAATTGAAGCTATGCAAGTCTTTTATATTATTAAATATTTAAAAGTAAACCTGT
ACTAATCTAA
NOV4a,
CG114555-01
Protein Sequence SEQ ID NO: 24 507 aa MW at 55327.3 kD
MARKQNRNSKELGLVPLTDDTSHAGPPGPGRALLECDHLRSGVPGGRRRKYIKAFYNESWERRHGRPI
DPDTLTLLWSVTVSIFAIGGLVGTLIVKMIGKVLGRKHTLLANNGFAISAALLMACSLQAGAFEMLIV
GRFIMGIDGGVALSVLPMYLSEISPKEIRGSLGQVTAIFICIGVFTGQLLGLPELLGKESTWPYLFGV
IVVPAVVQLLSLPFLPDSPRYLLLEKHNEARAVKAFQTFLGKADVSQEVEEVLAESRVQRSIRLVSVL
ELLRAPYVRWQVVTVIVTMACYQLCGLNAIWFYTNSIFGKAGIPLAKIPYVTLSTGGIETLAAVFSGL
VIEHLGRRPLLIGGFGLMGLFFGTLTITLTLQDHAPWVPYLSIVGILAIIASFCSGPGGIPFILTGEF
FQQSQRPAAFI IAGTVNWLSNFAVGLLFPFIQKSLDTYCFLVFATICITGAIYLYFVLPETKNRTYAE
ISQAFSKRNKAYPPEEKIDSAVTDGKINGRP
NOV4b,
247847074 SEQ ID NO: 25 1203 bp
DNA Sequence ORF Start at 1 ORF Stop: end of sequence
TTGTACAAAAAAGCAGGCTCCGCGGCCGCCCCCTTCACCGGTACCAGGAAGCACACTTTGCTGGCCAA
TAATGGGTTTGCAATTTCTGCTGCATTGCTGATGGCCTGCTCGCTCCAGGCAGGAGCCTTTGAAATGC
TCATCGTGGGACGTTTCATCATGGGCATAGATGGAGGCGTCGCCCTCAGTGTGCTCCCCATGTACCTC
AGTGAGATCTCACCCAAGGAGATCCGTGGCTCTCTGGGGCAGGTGACTGCCATCTTTATCTGCATTGG
CGTGTTCACTGGGCAGCTTCTGGGCCTGCCCGAGCTGCTGGGAAAGGAGAGTACCTGGCCATACCTGT
TTGGAGTGATTGTGGTCCCTGCCGTTGTCCAGCTGCTGAGCCTTCCCTTTCTCCCGGACAGCCCACGC
TACCTGCTCTTGGAGAAGCACAACGAGGCAAGAGCTGTGAAAGCCTTCCAAACGTTCTTGGGTAAAGC
AGACGTTTCCCAAGAGGTAGAGGAGGTCCTGGCTGAGAGCCGCGTGCAGAGGAGCATCCGCCTGGTGT
CCGTGCTGGAGCTGCTGAGAGCTCCCTACGTCCGCTGGCAGGTGGTCACCGTGATTGTCACCATGGCC
TGCTACCAGCTCTGTGGCCTCAATGCAATTTGGTTCTATACCAACAGCATCTTTGGAAAAGCTGGGAT
CCCTCCGGCAAAGATCCCATACGTCACCTTGAGTACAGGGGGCATCGAGACTTTGGCTGCCGTCTTCT
CTGGTTTGGTCATTGAGCACCTGGGACCGAGACCCCTCCTCATTGGTGGCTTTGGGCTCATGGGCCTC
TTCTTTGGGACCCTCACCATCACGCTGACCCTGCAGGACCACGCCCCCTGGGTCCCCTACCTGAGTAT
CGTGGGCATTCTGGCCATCATCGCCTCTTTCTGCAGTGGGCCAGGTGGCATCCCGTTCATCTTGACTG
GTGAGTTCTTCCAGCAATCTCAGCGGCCGGCTGCCTTCATCATTGCAGGCACCGTCAACTGGCTCTCC
AACTTTGCTGTTGGGCTCCTCTTCCCATTCATTCAGAAAAGTCTGGACACCTACTGTTTCCTAGTCTT
TGCTACAATTTGTATCACAGGTGCTATCTACCTGTATTTTGTGCTGCCTGAGACCAAAAAACAGAACCT
ATGCAGAAATCAGCCAGGCATTTCTCGAGGGCAAGGGTGGGCGCGCC
NOV4b,
247847074
Protein Sequence SEQ ID NO: 26 401 aa MW at 43391.7 kD
LYKKAGSAAAPFTGTRKHTLLANNGFAISAALLMACSLQAGAFEMLIVGRFIMGIDGGVALSVLPMYL
SEISPKEIRGSLGQVTAIFICIGVFTGQLLGLPELLGKESTWPYLFGVIVVPAVVQLLSLPFLPDSPR
YLLLEKHNEARAVKAFQTFLGKADVSQEVEEVLAESRVQRSIRLVSVLELLRAPYVRWQVVTVIVTMA
CYQLCGLNAIWFYTNSIFGKAGIPPAKIPYVTLSTGGIETLAAVFSGLVIEHLGRRPLLIGGFGLMGL
FFGTLTITLTLQDHAPWVPYLSIVGILAIIASFCSGPGGIPFILTGEFFQQSQRPAAFIIAGTVNWLS
NFAVGLLFPFIQKSLDTYCFLVFATICITGAIYLYFVLPETKNRTYAEISQAFLEGKGGRA
NOV4c,
247847070 SEQ ID NO: 27 1087 bp
DNA Sequence ORF Start: at 1 ORF Stop: end of sequence
GGCTCCGCGGCCGCCCCCTTCACCGGTACCAGGAAGCACACTTTGCTGGCCAATAATGGGTTTGCAA
TTTCTGCTGCATTGCTGATGGCCTGCTCGCTCCAGGCAGGAGCCTTTGAAATGCTCATCGTGGGACGT
TTCATCATGGGCATAGATGGAGGCGTCGCCCTCAGTGTGCTCCCCATGTACCTCAGTGAGATCTCACC
CAAGGAGATCCGTGGCTCTCTGGGGCAGGTGACTGCCATCTTTATCTGCATTGGCGTGTTCACTGGGC
AGCTTCTGGGCCTGCCCGAGCTGCTGGGAAAGGAGAGTACCTGGCCATACCTGTTTGGAGTGATTGTG
GTCCCTGCCGTTGTCCAGCTGCTGAGCCTTCCCTTTCTCCCGGACAGCCCACGCTACCTGCTCTTGGA
GAAGCACAACGAGGCAAGAGCTGTGAAAGCCTTCCAAACGTTCTTGGGTAAAGCAGACATTTCCCAAG
AGGTAGAGGAGGTCCTGGCTGAGAGCCGCGTGCAGAGGAGCATCCGCCTGGTGTCCGTGCTGGAGCTG
CTGAGAGCTCCCTACGTCCGCTGGCAGGTGGTCACCGTGATTGTCACCATGGCCTGCTACCAGCTCTG
TGGCCTCAATGCAATTTGGTTCTATACCAACAGCATCTTTGGAAAAGCTGGGATCCCTCCGGCAAAGA
TCCCATACGTCACCTTGAGTACAGGGGGCATCGAGACTTTGGCTGCCGTCTTCTCTGACCACGCCCCC
TGGGTCCCCTACCTGAGTATCGTGGGCATTCTGGCCATCATCGCCTCTTTCTGCAGTGGGCCAGGTGG
CATCCCGTTCATCTTGACTGGTGAGTTCTTCCAGCAATCTCAGCGGCCGGCTGCCTTCATCATTGCAG
GCACCGTCAACTGGCTCTCCAACTTTGCTGTTGGGCTCCTCTTCCCATTCATTCAGAAAAGTCTGGAC
ACCTACTGTTTCCTAGTCTTTGCTACAATTTGTATCACAGGTGCTATCTACCTGTATTTTGTGCTGCC
TGAGACCAAAAACAGAACCTATGCAGAAATCAGCCAGGCATTTCTCGAGGGCAAGGGTGGGCGCGCC
NOV4c,
247847070
Protein Sequence SEQ ID NO: 28 362 aa MW at 39164.5 kD
GSAAAPFTGTRKHTLLANNGFAISAALLMACSLQAGAFEMLIVGRFIMGIDGGVALSVLPMYLSEISP
KEIRGSLGQVTAIFICIGVFTGQLLGLPELLGKESTWPYLFGVIVVPAVVQLLSLPFLPDSPRYLLLE
KHNEARAVKAFQTFLGKADISQEVEEVLAESRVQRSIRLVSVLELLRAPYVRWQVVTVIVTMACYQLC
GLNAIWFYTNSIFGKAGIPPAKIPYVTLSTGGIETLAAVFSDHAPWVPYLSIVGILAIIASFCSGPGG
IPFILTGEFFQQSQRPAAFIIAGTVNWLSNFAVGLLFPFIQKSLDTYCFLVFATICITGAIYLYFVLP
ETKNRTYAEISQAFLEGKGGRA
NOV4d,
247847055 SEQ ID NO: 29 1189 bp
DNA Sequence ORF Start: at 1 ORF Stop: end of sequence
GGCTCCGCGGCCGCCCCCTTCACCGGTACCAGGAAGCACACTTTGCTGGCCAATAATGGGTTTGCAA
TTTCTGCTGCATTGCTGATGGCCTGCTCGCTCCAGGCAGGAGCCTTTGAAATGCTCATCGTGGGACGC
TTCATCATGGGCATAGATGGAGGCGTCGCCCTCAGTGTGCTCCCCATGTACCTCAGTGAGATCTCACC
CAAGGAGATCCGTGGCTCTCTGGGGCAGGTGACTGCCATCTTTATCTGCATTGGCGTGTTCACTGGGC
AGCTTCTGGGCCTGCCCGAGCTGCTGGGAAAGGAGAGTACCTGGCCATACCTGTTTGGAGTGATTGTG
GTCCCTGCCGTTGTCCAGCTGCTGAGCCTTCCCTTTCTCCCGGACAGCCCACGCTACCTGCTCTTGGA
GAAGCACAACGAGGCAAGAGCTGTGAAAGCCTTCCAAACGTTCTTGGGTAAAGCAGACGTTTCCCAAG
AGGTAGAGGAGGTCCTGGCTGAGAGCCACGTGCAGAGGAGCATCCGCCTGGTGTCCGTGCTGGAGCTG
CTGAGAGCTCCCTACGTCCGCTGGCAGGTGGTCACCGTGATTGTCACCATGGCCTGCTACCAGCTCTG
TGGCCTCAATGCAATTTGGTTCTATACCAACAGCATCTTTGGAAAAGCTGGGATCCCTCCGGCAAAGA
TCCCATACGTCACCTTGAGTACAGGGGGCATCGAGACTTTGGCTGCCGTCTTCTCTGGTTTGGTCATT
GAGCACCTGGGACGGAGACCCCTCCTCATTGGTGGCTTTGGGCTCATGGGCCTCTTCTTTGGGACCCT
CACCATCACGCTGACCCTGCAGGACCACGCCCCCTGGGTCCCCTACCTGAGTATCGTGGGCATTCTGG
CCATCATCGCCTCTTTCTGCAGTGGGCCAGGTGGCATCCCGTTCATCTTGACTGGTGAGTTCTTCCAG
CAATCTCAGCGGCCGGCTGCCTTCATCATTGCAGGCACCGTCAACTGGCTCTCCAACTTTGCTGTTGG
GCTCCTCTTCCCATTCATTCAGAAAAAGTCTGGACACCTACTGTTTCCTAGTCTTTGCTACAATTTGTA
TCACAGGTGCTATCTACCTGTATTTTGTGCTGCCTGAGACCAAAAACAGAACCTATGCAGAAATCAGC
CAGGCATTTCTCGAGGGCAAGGGTGGGCGCGCC
NOV4d,
247847055
Protein Sequence SEQ ID NO: 30 396 aa MW at 42768.9 kD
GSAAAPFTGTRKHTLLANNGFAISAALLMACSLQAGAFEMLIVGRFIMGIDGGVALSVLPMYLSEISP
KEIRGSLGQVTAI FICIGVFTGQLLGLPELLGKESTWPYLFGVIVVPAVVQLLSLPFLPDSPRYLLLE
KHNEARAVKAFQTFLGKADVSQEVEEVLAESHVQRSIRLVSVLELLRAPYVRWQVVTVIVTMACYQLC
GLNAIWFYTNSIFGKAGIPPAKIPYVTLSTGGIETLAAVFSGLVIEHLGRRPLLIGGFGLMGLFFGTL
TITLTLQDHAPWVPYLSIVGILAIIASFCSCPGGIPFILTGEFFQQSQRPAAFIIAGTVNWLSNFAVG
LLFPFIQKSLDTYCFLVFATICITGAIYLYFVLPETKNRTYAEISQAFLEGKGGRA
NOV4e,
247847059 SEQ ID NO: 31 1189 bp
DNA Sequence ORF Start: at 1 ORF Stop: end of sequence
GGCTCCGCGGCCGCCCCCTTCACCGGTACCAGGAAGCACACTTTGCTGGCCAATAATGGGTTTGCAA
TTTCTGCTGCATTGCTGATGGCCTGCTCGCTCCAGGCAGGAGCCTTTGAAATGCTCATCGTGGGACGC
TTCATCATGGGCATAGATGGAGGCGTCGCCCTCAGTGTGCTCCCCATGTACCTCAGTGAGATCTCACC
CAAGGAGATCCGTGGCTCTCTGGGGCAGGTGACTGCCATCTTTATCTGCATTGGCGTGTTCACTGGGC
AGCTTCTGGGCCTGCCCGAGCTGCTGGGAAAGGAGAGTACCTGGCCATACCTGTTTGGAGTGATTGTG
GTCCCTGCCGTTGTCCAGCTGCTGAGCCTTCCCTTTCTCCCGGACAGCCCACGCTACCTGCTCTTGGA
GAAGCACAACGAGGCAAGAGCTGTGAAAGCCTTCCAAACGTTCTTGGGTAAAGCAGACATTTCCCAAG
AGGTAGAGGAGGTCCTGGCTGAGAGCCGCGTGCAGAGGAGCATCCGCCTGGTGTCCGTGCTGGAGCTG
CTGAGAGCTCCCTACGTCCGCTGGCAGGTGGTCACCGTGATTGTCACCATGGCCTGCTACCAGCTCTG
TGGCCTCAATGCAATTTGGTTCTATACCAACAGCATCTTTGGAAAAGCTGGGATCCCTCCGGCAAAGA
TCCCATACGTCACCTTGAGTACAGGGGGCATCGAGACTTTGGCTGCCGTCTTCTCTGGTTTGGTCATT
GAGCACCTGGGACGGAGACCCCTCCTCATTGGTGGCTTTGGGCTCATGGGCCTCTTCTTTGGGACCCT
CACCATCACGCTGACCCTGCAGGACCACGCCCCCTGGGTCCCCTACCTGAGTATCGTGGGCATTCTGG
CCATCATCGCCTCTTTCTGCAGTGGGCCAGGTGGCATCCCGTTCATCTTGACTGGTGAGTTCTTCCAG
CAATCTCAGCGGCCGGCTGCCTTCATCATTGCAGGCACCGTCAACTGGCTCTCCAACTTTGCTGTTGG
GCTCCTCTTCCCATTCATTCAGAAAAGTCTGGACACCTACTGTTTCCTAGTCTTTGCTACAATTTGTA
TCACAGGTGCTATCTACCTGTATTTTGTGCTGCCTGAGACCAAAAACAGAACCTATGCAGAAATCAGC
CAGGCATTTCTCGAGGGCAAGGGTGGGCGCGCC
NOV4e,
247847059
Protein Sequence SEQ ID NO: 32 396 aa MW at 42801.9 kD
GSAAAPFTGTRKHTLLANNGFAISAALLMACSLQAGAFEMLIVGRFIMGIDGGVALSVLPMYLSEISP
KEIRGSLGQVTAIFICIGVFTGQLLGLPELLGKESTWPYLFGVIVVPAVVQLLSLPFLPDSPRYLLLE
KHNEARAVKAFQTFLGKADISQEVEEVLAESRVQRSIRLVSVLELLRAPYVRWQVVTVIVTMACYQLC
GLNAIWFYTNSIFGKAGIPPAKIPYVTLSTGGIETLAAVFSGLVIEHLGRRPLLIGGFGLMGLFFGTL
TITLTLQDHAPWVPYLSIVGILAIIASFCSGPGGIPFILTGEFFQQSQRPAAFIIAGTVNWLSNFAVG
LLFPFIQKSLDTYCFLVFATICITGAIYLYFVLPETKNRTYAEISQAFLEGKGGRA
NOV4f,
247847047 SEQ ID NO: 33 1189 bp
DNA Sequence ORF Start: at 1 ORF Stop: end of sequence
GGCTCCGCGGCCGCCCCCTTCACCGGTACCAGGAAGCACACTTTGCTGGCCAATAATGGGTTTGCAA
TTTCTGCTGCATTGCTGATGGCCTGCTCGCTCCAGGCAGGAGCCTTTGAAATGCTCATCGTGGGACGC
TTCATCATGGGCATAGATGGAGGCGTCGCCCTCAGTGTGCTCCCCATGTACCTCAGTGAGATCTCACC
CAAGGAGATCCGTGGCTCTCTGGGGCAGGTGACTGCCATCTTTATCTGCATTGGCGTGTTCACTGGGC
AGCTTCTGGGCCTGCCCGAGCTGCTGGGAAAGGAGAGTACCTGGCCATACCTGTTTGGAGTGATTGTG
GTCCCTGCCGTTGTCCAGCTGCTGAGCCTTCCCTTTCTCCCGGACAGCCCACGCTACCTGCTCTTGGA
GAAGCACAACGAGGCAAGAGCTGTGAAAGCCTTCCAAACGTTCTTGGGTAAAGCAGACGTTTCCCAAG
AGGTAGAGGAGGTCCTGGCTGAGAGCCGCGTGCAGAGGAGCATCCGCCTGGTGTCCGTGCTGGAGCTG
CTGAGAGCTCCCTACGTCCGCTGGCAGGTGGTCACCGTGATTGTCACCATGGCCTGCTACCAGCTCTG
TGGCCTCAATGCAATTTGGTTCTATACCAACAGCATCTTTGGAAAAGCTGGGATCCCTCTGGCAAAGA
TCCCATACGTCACCTTGAGTACAGGGGGCATCGAGACTTTGGCTGCCGTCTTCTCTGGTTTGGTCATT
GAGCACCTGGGACGGAGACCCCTCCTCATTGGTGGCTTTGGGCTCATGGGCCTCTTCTTTGGGACCCT
CACCATCACGCTGACCCTGCAGGACCACGCCCCCTGGGTCCCCTACCTGAGTATCGTGGGCATTCTGG
CCATCATCGCCTCTTTCTGCAGTGGGCCAGGTGGCATCCCGTTCATCTTGACTGGTGAGTTCTTCCAG
CAATCTCAGCGGCCGGCTGCCTTCATCATTGCAGGCACCGTCAACTGGCTCTCCAACTTTGCTGTTGG
GCTCCTCTTCCCATTCATTCAGAAAAGTCTGGACACCTACTGTTTCCTAGTCTTTGCTACAATTTGTA
TCACAGGTGCTATCTACCTGTATTTTGTGCTGCCTGAGACCAAAAACAGAACCTATGCAGAAATCAGC
CAGGCATTTCTCGAGGGCAAGGGTGGGCGCGCC
NOV4f,
247847047
Protein Sequence SEQ ID NO: 34 1396 aa MW at 42803.9 kD
GSAAAPFTGTRKHTLLANNGFAISAALLMACSLQAGAFEMLIVGRFIMGIDGGVALSVLPMYLSEISP
KEIRGSLGQVTAIFICIGVFTGQLLGLPELLGKESTWPYLFGVIVVPAVVQLLSLPFLPDSPRYLLLE
KHNEARAVKAFQTFLGKADVSQEVEEVLAESRVQRSIRLVSVLELLRAPYVRWQVVTVIVTMACYQLC
GLNAIWFYTNSIFGKAGIPLAKIPYVTLSTGGIETLAAVFSGLVIEHLGRRPLLIGGFGLMGLFFGTL
TITLTLQDHAPWVPYLSIVGILAIIASFCSGPGGIPFILTGEFFQQSQRPAAFIIAGTVNWLSNFAVG
LLFPFIQKSLDTYCFLVFATICITGAIYLYFVLPETKNRTYAEISQAFLEGKGGRA
NOV4g,
CG114555-02 SEQ ID NO: 35 1682 bp
DNA Sequence ORF Start: ATG at 14 ORF Stop: TAA at 1634
GTCACTGAGACCCATGGCAAGGAAACAAAATAGGAATTCCAAGGAACTGGGCCTAGTTCCCCTCACAG
ATGACACCAGCCACGCCAGGCCTCCAGGGCCAGGGAGGGCACTGCTGGAGTGTGTCCACCTGAGGAGT
GGGGTGCCAGGTGGAAGGAGAAGAAAGGACTGGTCCTGCTCGCTCCTCGTGGCCTCCCTCGCGGGCGC
CTTCGGCTCCCCCTTCCTCTACGGCTACAACCTGTCGGTGGTGAATGCCCCCACCCCGTACATCAAGG
CCTTTTACAATGAGTCATGGGAAAGAAGGCATGGACGTCCAATAGACCCAGACACTCTGACTCTGCTC
TGGTCTGTGACTGTGTCCATATTCGCCATCGGTGGACTTGTGGGGACATTAATTGTGAAGATGATTGG
AAAGGTTCTTGGGAGGAAGCACACTTTGCTGGCCAATAATGGGTTTGCAATTTCTGCTGCATTGCTGA
TGGCCTGCTCGCTCCAGGCAGGAGCCTTTGAGATGCTCATCGTGGGACGCTTCATCATGGGCATAGAT
GGAGGCGTCGCCCTCAGTGTGCTCCCCATGTACCTCAGTGAGATCTCACCCAAGGAGATCCGTGGCTC
TCTGGGGCAGGTGACTGCCATCTTTATCTGCATTGGCGTGTTCACTGGGCAGCTTCTGGGCCTGCCCG
AGCTGCTGGGAAAGGAGAGTACCTGGCCATACCTGTTTGGAGTGATTGTGGTCCCTGCCGTTGTCCAG
CTGCTGAGCCTTCCCTTTCTCCTGGACAGCCCACGCTACCTGCTCTTGGAGAAGCACAACGAGGCAAG
AGCTGTGAAAGCCTTCCAAACGTTCTTGGGTAAAGCAGACGTTTCCCAAGAGGTAGAGGAGGTCCTGG
CTGAGAGCCGCGTGCAGAGGAGCATCCGCCTGGTGTCCGTGCTGGAGCTGCTGAGAGCTCCCTACGTC
CGCTGGCAGGTGGTCACCGTGATTGTCACCATGGCCTGCTACCAGCTCTGTGGCCTCAATGCAATTTG
GTTCTATACCAACAGCATCTTTGGAAAAGCTGGGATCCCTCTGGCAAAGATCCCATACGTCACCTTGA
GTACAGGGGGCATCGAGACTTTGGCTGCCGTCTTCTCTGGTTTGGTCATTGAGCACCTGGGACGGAGA
CCCCTCCTCATTGGTGGCTTTGGGCTCATGGGCCTCTTCTTTGGGGCCCTCACCATCACGCTGACCCT
GCAGGACCACGCCCCCTGGGTCCCCTACCTGAGTATCGTGGGCATTCTGGCCATCATCGCCTCTTTCT
GCAGTGGGCCAGGTGGCATCCCGTTCATCTTGACTGGTGAGTTCTTCCAGCAATCTCAGCGGCCGGCT
GCCTTCATCATTGCAGGCACCGTCAACTGGCTCTCCAACTTTGCTGTTGGGCTCCTCTTCCCATTCAT
TCAGAAAAGTCTGGACACCTACTGTTTCCTAGTCTTTGCTACAATTTGTATCACAGGTGCTATCTACC
TGTATTTTGTGCTGCCTGAGACCAAAAACAGAACCTATGCAGAAATCAGCCAGGCATTTTCCAAAAGG
AACAAAGCATACCCACCAGAAGAGAAAATCGACTCAGCTGTCACTGATGGTAAGATAAATGGAAGGCC
TTAACAAGTTTCCTCCTCCACGTTGGACAATTATGTCAAAAACAGGATTG
NOV4g,
CG114555-02
Protein Sequence SEQ ID NO: 36 540 aa MW at 58796.3 kD
MARKQNRNSKELGLVPLTDDTSHARPPGPGRALLECVHLRSGVPGGRRRKDWSCSLLVASLAGAFGSP
FLYGYNLSVVNAPTPYIKAFYNESWERRHGRPIDPDTLTLLWSVTVSIFAIGGLVGTLIVKMIGKVLG
RKHTLLANNGFAISAALLMACSLQAGAFEMLIVGRFIMGIDGGVALSVLPMYLSEISPKEIRGSLGQV
TAIFICIGVFTGQLLGLPELLGKESTWPYLFGVIVVPAVVQLLSLPFLLDSPRYLLLEKHNEARAVKA
FQTFLGKADVSQEVEEVLAESRVQRSIRLVSVLELLRAPYVRWQVVTVIVTMACYQLCGLNAIWFYTN
SIFGKAGIPLAKIPYVTLSTGGIETLAAVFSGLVIEHLGRRPLLIGGFGLMGLFFGALTITLTLQDHA
PWVPYLSIVGILAIIASFCSGPGGIPFILTGEFFQQSQRPAAFIIAGTVNWLSNFAVGLLFPFIQKSL
DTYCFLVFATICITGAIYLYFVLPETKNRTYAEISQAFSKRNKAYPPEEKIDSAVTDGKINGRP
NOV4h,
CG114555-03 SEQ ID NO: 37 1757 bp
DNA Sequence ORF Start: ATG at 14 ORF Stop: TAA at 1709
GTCACTGAGACCCATGGCAAGGAAGCAAAATAGGAATTCCAAGGAACTGGGCCTAGTTCCCCTCACAG
ATGACACCAGCCACGCCGGGCCTCCAGGGCCAGGGAGGGCACTGCTGGAGTGTGACCACCTGAGGAGT
GGGGTGCCAGGTGGAAGGAGAAGAAAGCAGCCTCTACGGAGCACCTCCTCTGCAGCAGGCTCCTCAAC
AACATATGTGGCCAGTGCTGCTATTAAGATCCCATTTCACAGGTGGGCAAGCTTAGCCCCAGAAAAGT
CAAGTCACTTGCTCAGACTCCTACAGCTGAGGGGACTGGCCCTGGAGGTAAAGCTGATATCACTTGGC
TCAAAGCCCCAAAGCTCTATCTCGTGGCTGGTGGCACTAGAGGAGACAAACGAGATTGGCAGAGACTG
GTCCTGCTCGCTCCTCGTGGCCTCCCTCGCGGGCGCCTTCGGCTCCTCCTTCCTCTACGGCTACAACC
TGTCGGTGGTGAATGCCCCCACCCCGCACACTTTGCTGGCCAATAATGGGTTTGCAATTTCTGCTGCA
TTGCTGATGGCCTGCTCGCTCCAGGCAGGAGCCTTTGAAATGCTCATCGTGGGACGCTTCATCATGGG
CATAGATGGAGGCGTCGCCCTCAGTGTGCTCCCCATGTACCTCAGTGAGATCTCACCCAAGGAGATCC
GTGGCTCTCTGGGGCAGGTGACTGCCATCTTTATCTGCATTGGCGTGTTCACTGGGCAGCTTCTGGGC
CTGCCCGAGCTGCTGGGAAAGGAGAGTACCTGGCCATACCTGTTTGGAGTGATTGTGGTCCCTGCCGT
TGTCCAGCTGCTGAGCCTTCCCTTTCTCCTGGACAGCCCACGCTACCTGCTCTTGGAGAAGCACAACG
AGGCAAGAGCTGTGAAAGCCTTCCAAACGTTCTTGGGTAAAGCAGACGTTTCCCAAGAGGTAGAGGAG
GTCCTGGCTGAGAGCCGCGTGCAGAGGAGCATCCGCCTGGTGTCCGTGCTGGAGCTGCTGAGAGCTCC
CTACGTCCGCTGGCAGGTGGTCACCGTGATTGTCACCATGGCCTGCTACCAGCTCTGTGGCCTCAATG
CAATTTGGTTCTATACCAACAGCATCTTTGGAAAAGCTGGGATCCCTCTGGCAAAGATCCCATACGTC
ACCTTGAGTACAGGGGGCATCGAGACTTTGGCTGCCGTCTTCTCTGGTTTGGTCATTGAGCACCTGGG
ACGGAGACCCCTCCTCATTGGTGGCTTTGGGCTCATGGGCCTCTTCTTTGGGGCCCTCACCATCACGC
TGACCCTGCAGGACCACGCCCCCTGGGTCCCCTACCTGAGTATCGTGGGCATTCTGGCCATCATCGCC
TCTTTCTGCAGTGGGCCAGGTGGCATCCCGTTCATCTTGACTGGTGAGTTCTTCCAGCAATCTCAGCG
GCCGGCTGCCTTCATCATTGCAGGCACCGTCAACTGGCTCTCCAACTTTGCTGTTGGGCTCCTCTTCC
CATTCATTCAGAAAAGTCTGGACACCTACTGTTTCCTAGTCTTTGCTACAATTTGTATCACAGGTGCT
ATCTACCTGTATTTTGTGCTGCCTGAGACCAAAAAACAGAACCTATGCAGAAATCAGCCAGGCATTTTC
CAAAAGGAACAAAGCATACCCACCAGAAGAGAAAATCGACTCAGCTGTCACTGATGGTAAGATAAATG
GAAGGCCTTAACAAGTTTCCTCCTCCACGTTGGACAATTATGTCAAAAACAGGATTG
NOV4h,
CG114555-03
Protein Sequence SEQ ID NO: 38 565 aa MW at 61112.6 kD
MARKQNRNSKELGLVPLTDDTSHAGPPGPGRALLECDHLRSGVPGGRRRKQPLRSTSSAAGSSTTYVA
SAAIKIPFHRWASLAPEKSSHLLRLLQLRGLALEVKLISLGSKPQSSISWLVALEETNEIGRDWSCSL
LVASLAGAFGSSFLYGYNLSVVNAPTPHTLLANNGFAISAALLMACSLQAGAFEMLIVGRFIMGIDGG
VALSVLPMYLSEISPKEIRGSLGQVTAIFICIGVFTGQLLGLPELLGKESTWPYLFGVIVVPAVVQLL
SLPFLLDSPRYLLLEKHNEARAVKAFQTFLGKADVSQEVEEVLAESRVQRSIRLVSVLELLRAPYVRW
QVVTVIVTMACYQLCGLNAIWFYTNSIFGKAGIPLAKIPYVTLSTGGIETLAAVFSGLVIEHLGRRPL
LIGGFGLMGLFFGALTITLTLQDHAPWVPYLSIVGILAIIASFCSGPGGIPFILTGEFFQQSQRPAAF
IIAGTVNWLSNFAVGLLFPFIQKSLDTYCFLVFATICITGAIYLYFVLPETKNRTYAEISQAFSKRNK
AYPPEEKIDSAVTDGKINGRP
NOV4i,
CG114555-04 SEQ ID NO: 39 1502 bp
DNA Sequence ORF Start: ATG at 14 ORF Stop: TAA at 1454
GTCACTGAGACCCATGGCAAGGAAACAAAATAGGAATTCCAAGGAACTGGGCCTAGTTCCCCTCACAG
ATGACACCAGCCACGCCAGGCCTCCAGGGCCAGGGAGGGCACTGCTGGAGTGTGTCCACCTGAGGAGT
GGGGTGCCAGGTGGAAGGAGAAGAAAGGACTGGTCCTGCTCGCTCCTCGTGGCCTCCCTCGCGGGCGC
CTTCGGCTCCCCCTTCCTCTACGGCTACAACCTGTCGGTGGTGAATGCCCCCACCCCGTACATCAAGG
CCTTTTACAATCAGTCATGGGAAAGAAGGCATGGACGTCCAATAGACCCAGACACTCTGACTCTGCTC
TGGTCTGTGACTGTGTCCATATTCGCCATCGGTGGACTTGTGGGGACATTAATTGTGAAGATGATTGG
AAAGGTTCTTGGGAGGAAGCACACTTTGCTGGCCAATAATGGGTTTGCAATTTCTGCTGCATTGCTGA
TGGCCTGCTCGCTCCAGGCAGGAGCCTTTGAGATGCTCATCGTGGGACGCTTCATCATGGGCATAGAT
GGAGGCGTCGCCCTCAGTGTGCTCCCCATGTACCTCAGTGAGATCTCACCCAAGGAGATCCGTGGCTC
TCTGGGGCAGGTGACTGCCATCTTTATCTGCATTGGCGTGTTCACTGGGCAGCTTCTGGGCCTGCCCG
AGCTGCTGGGAAAGGAGAGTACCTGGCCATACCTGTTTGGAGTGATTGTGGTCCCTGCCGTTGTCCAG
CTGCTGAGCCTTCCCTTTCTCCTGGACAGCCCACGCTACCTGCTCTTGGAGAAGCACAACGAGGCAAG
AGCTGTGAAAGCCTTCCAAACGTTCTTGGGTAAAGCAGACGTTTCCCAAGAGGTAGAGGAGGTCCTGG
CTGAGAGCCGCGTGCAGAGGAGCATCCGCCTGGTGTCCGTGCTGGAGCTGCTGAGAGCTCCCTACGTC
CGCTGGCAGGTGGTCACCGTGATTGTCACCATGGCCTGCTACCAGCTCTGTGGCCTCAATGCAATTTG
GTTCTATACCAACAGCATCTTTGGAAAAGCTGGGATCCCTCTGGCAAAGATCCCATACGTCACCTTGA
GTACAGGGGGCATCGAGACTTTGGCTGCCGTCTTCTCTGGCATCCCGTTCATCTTGACTGGTGAGTTC
TTCCAGCAATCTCAGCGGCCGGCTGCCTTCATCATTGCAGGCACCGTCAACTGGCTCTCCAACTTTGC
TGTTGGGCTCCTCTTCCCATTCATTCAGAAAAGTCTGGACACCTACTGTTTCCTAGTCTTTGCTACAA
TTTGTATCACAGGTGCTATCTACCTGTATTTTGTGCTGCCTGAGACCAAAAACAGAACCTATGCAGAA
ATCAGCCAGGCATTTTCCAAAAGGAACAAAGCATACCCACCAGAAGAGAAAATCGACTCAGCTGTCAC
TGATGGTAAGATAAATGGAAGGCCTTAACAAGTTTCCTCCTCCACGTTGGACAATTATGTCAAAAACA
GGATTG
NOV4i,
CG114555-04
Protein Sequence SEQ ID NO: 40 480 aa MW at 52522.9 kD
MARKQNRNSKELGLVPLTDDTSHARPPGPGRALLECVHLRSGVPGGRRRKDWSCSLLVASLAGAFGSP
FLYGYNLSVVNAPTPYIKAFYNESWERRHGRPIDPDTLTLLWSVTVSIFAIGGLVGTLIVKMIGKVLG
RKHTLLANNGFAISAALLMACSLQAGAFEMLIVGRFIMGIDGGVALSVLPMYLSEISPKEIRGSLGQV
TAIFICIGVFTGQLLGLPELLGKESTWPYLFGVIVVPAVVQLLSLPFLLDSPRYLLLEKHNEARAVKA
FQTFLGKADVSQEVEEVLAESRVQRSIRLVSVLELLRAPYVRWQVVTVIVTMACYQLCGLNAIWFYTN
SIFGKAGIPLAKIPYVTLSTGGIETLAAVFSGIPFILTGEFFQQSQRPAAFIIAGTVNWLSNFAVGLL
FPFIQKSLDTYCFLVFATICITGAIYLYFVLPETKNRTYAEISQAFSKRNKAYPPEEKIDSAVTDGKI
NGRP
NOV4j,
13379365 SNP in
CG114555-01 SEQ ID NO: 41 SNP: G/A at position 86
DNA Sequence ORF Start: ATG at 14 ORF Stop: TAA at 1535
GTCACTGAGACCCATGGCAAGGAAACAAAATAGGAATTCCAAGGAACTGGGCCTAGTTCCCCTCACAGATGA
CACCAGCCACGCCAGGCCTCCAGGGCCAGGGAGGGCACTGCTGGAGTGTGACCACCTGAGGAGTGGGGTGCC
AGGTGGAAGGAGAAGAAAGTACATCAAGGCCTTTTACAATGAGTCATGGGAAAGAAGGCATGGACGTCCAAT
AGACCCAGACACTCTGACTCTGCTCTGGTCTGTGACTGTGTCCATATTCGCCATCGGTGGACTTGTGGGGAC
ATTAATTGTGAAGATGATTGGAAAGGTTCTTGGGAGGAAGCACACTTTGCTGGCCAATAATGGGTTTGCAAT
TTCTGCTGCATTGCTGATGGCCTGCTCGCTCCAGGCAGGAGCCTTTGAAATGCTCATCGTGGGACGCTTCAT
CATGGGCATAGATGGAGGCGTCGCCCTCAGTGTGCTCCCCATGTACCTCAGTGAGATCTCACCCAAGGAGAT
CCGTGGCTCTCTGGGGCAGGTGACTGCCATCTTTATCTGCATTGGCGTGTTCACTGGGCAGCTTCTGGGCCT
GCCCGAGCTGCTGGGAAAGGAGAGTACCTGGCCATACCTGTTTGGAGTGATTGTGGTCCCTGCCGTTGTCCA
GCTGCTGAGCCTTCCCTTTCTCCCGGACAGCCCACGCTACCTGCTCTTGGAGAAGCACAACGAGGCAAGAGC
TGTGAAAGCCTTCCAAACGTTCTTGGGTAAAGCAGACGTTTCCCAAGAGGTAGAGGAGGTCCTGGCTGAGAG
CCGCGTGCAGAGGAGCATCCGCCTGGTGTCCGTGCTGGAGCTGCTGAGAGCTCCCTACGTCCGCTGGCAGGT
GGTCACCGTGATTGTCACCATGGCCTGCTACCAGCTCTGTGGCCTCAATGCAATTTGGTTCTATACCAACAG
CATCTTTGGAAAAGCTGGGATCCCTCTGGCAAAGATCCCATACGTCACCTTGAGTACAGGGGGCATCGAGAC
TTTGGCTGCCGTCTTCTCTGGTTTGGTCATTGAGCACCTGGGACGGAGACCCCTCCTCATTGGTGGCTTTGG
GCTCATGGGCCTCTTCTTTGGGACCCTCACCATCACGCTGACCCTGCAGGACCACGCCCCCTGGGTCCCCTA
CCTGAGTATCGTGGGCATTCTGGCCATCATCGCCTCTTTCTGCAGTGGGCCAGGTGGCATCCCGTTCATCTT
GACTGGTGAGTTCTTCCAGCAATCTCAGCGGCCGGCTGCCTTCATCATTGCAGGCACCGTCAACTGGCTCTC
CAACTTTGCTGTTGGGCTCCTCTTCCCATTCATTCAGAAAAGTCTGGACACCTACTGTTTCCTA
GTCTTTGCTACAATTTGTATCACAGGTGCTATCTACCTGTATTTTGTGCTGCCTGAGACCAAAAACAGAACC
TATGCAGAAATCAGCCAGGCATTTTCCAAAAGGAACAAAGCATACCCACCAGAAGAGAAAATCGACTCAGCT
GTCACTGATGGTAAGATAAATGGAAGGCCTTAACAAGTTTCCTCCTCCACGTTGGACAATTATGTCAAAAAC
AGGATTGTCTACATGGATGATCTCACTTTTCAGGAAACTTAAAATTTACCCATTATTGGGAAGCTTAAATGA
ATTGAAGCTATGCAAGTCTTTTATATTATTAAATATTTAAAAGTAAACCTGTACTAATCTAA
NOV4j,
13379365 SNP in
CG114555-01 SNP: Gly to Arg
Protein Sequence SEQ ID NO: 42 507 aa at position 25
MARKQNRNSKELGLVPLTDDTSHARPPGPGRALLECDHLRSGVPGGRRRKYIKAFYNESWERRHGRPIDPDT
LTLLWSVTVSIFAIGGLVGTLIVKMIGKVLGRKHTLLANNGFAISAALLMACSLQAGAFEMLIVGRFIMGID
GGVALSVLPMYLSEISPKEIRGSLGQVTAIFICIGVFTGQLLGLPELLGKESTWPYLFGVIVVPAVVQLLSL
PFLPDSPRYLLLEKHNEARAVKAFQTFLGKADVSQEVEEVLAESRVQRSIRLVSVLELLRAPYVRWQVVTVI
VTMACYQLCGLNAIWFYTNSIFGKAGIPLAKIPYVTLSTGGIETLAAVFSGLVIEHLGRRPLLIGGFGLMGL
FFGTLTITLTLQDHAPWVPYLSIVGILAIIASFCSGPGGIPFILTGEFFQQSQRPAAFIIAGTVNWLSNFAV
GLLFPFIQKSLDTYCFLVFATICITGAIYLYFVLPETKNRTYAEISQAFSKRNKAYPPEEKIDSAVTDGKIN
GRP
NOV4k,
13379364 SNP in
CG114555-01 SEQ ID NO: 43 SNP: G/A at position 97
DNA Sequence ORF Start: ATG at 14 ORF Stop: TAA at 1535
GTCACTGAGACCCATGGCAAGGAAACAAAATAGGAATTCCAAGGAACTGGGCCTAGTTCCCCTCACAGATGA
CACCAGCCACGCCGGGCCTCCAGGACCAGGGAGGGCACTGCTGGAGTGTGACCACCTGAGGAGTGGGGTGCC
AGGTGGAAGGAGAAGAAAGTACATCAAGGCCTTTTACAATGAGTCATGGGAAAGAAGGCATGGACGTCCAAT
AGACCCAGACACTCTGACTCTGCTCTGGTCTGTGACTGTGTCCATATTCGCCATCGGTGGACTTGTGGGGAC
ATTAATTGTGAAGATGATTGGAAAGGTTCTTGGGAGGAAGCACACTTTGCTGGCCAATAATGGGTTTGCAAT
TTCTGCTGCATTGCTGATGGCCTGCTCGCTCCAGGCAGGAGCCTTTGAAATGCTCATCGTGGGACGCTTCAT
CATGGGCATAGATGGAGGCGTCGCCCTCAGTGTGCTCCCCATGTACCTCAGTGAGATCTCACCCAAGGAGAT
CCGTGGCTCTCTGGGGCAGGTGACTGCCATCTTTATCTGCATTGGCGTGTTCACTGGGCAGCTTCTGGGCCT
GCCCGAGCTGCTGGGAAAGGAGAGTACCTGGCCATACCTGTTTGGAGTGATTGTGGTCCCTGCCGTTGTCCA
GCTGCTGAGCCTTCCCTTTCTCCCGGACAGCCCACGCTACCTGCTCTTGGAGAAGCACAACGAGGCAAGAGC
TGTGAAAGCCTTCCAAACGTTCTTGGGTAAAGCAGACGTTTCCCAAGAGGTAGAGGAGGTCCTGGCTGAGAG
CCGCGTGCAGAGGAGCATCCGCCTGGTGTCCGTGCTGGAGCTGCTGAGAGCTCCCTACGTCCGCTGGCAGGT
GGTCACCGTGATTGTCACCATGGCCTGCTACCAGCTCTGTGGCCTCAATGCAATTTGGTTCTATACCAACAG
CATCTTTGGAAAAGCTGGGATCCCTCTGGCAAAGATCCCATACGTCACCTTGAGTACAGGGGGCATCGAGAC
TTTGGCTGCCGTCTTCTCTGGTTTGGTCATTGAGCACCTGGGACGGAGACCCCTCCTCATTGGTGGCTTTGG
GCTCATGGGCCTCTTCTTTGGGACCCTCACCATCACGCTGACCCTGCAGGACCACGCCCCCTGGGTCCCCTA
CCTGAGTATCGTGGGCATTCTGGCCATCATCGCCTCTTTCTGCAGTGGGCCAGGTGGCATCCCGTTCATCTT
GACTGGTGAGTTCTTCCAGCAATCTCAGCGGCCGGCTGCCTTCATCATTGCAGGCACCGTCAACTGGCTCTC
CAACTTTGCTGTTGGGCTCCTCTTCCCATTCATTCAGAAAAGTCTGGACACCTACTGTTTCCTAGTCTTTGC
TACAATTTGTATCACAGGTGCTATCTACCTGTATTTTGTGCTGCCTGAGACCAAAAACAGAACCTATGCAGA
AATCAGCCAGGCATTTTCCAAAAGGAACAAAGCATACCCACCAGAAGAGAAAATCGACTCAGCTGTCACTGA
TGGTAAGATAAATGGAAGGCCTTAACAAGTTTCCTCCTCCACGTTGGACAATTATGTCAAAAACAGGATTGT
CTACATGGATGATCTCACTTTTCAGGAAACTTAAAATTTACCCATTATTGGGAAGCTTAAATGAATTGAAGC
TATGCAAGTCTTTTATATTATTAAATATTTAAAAGTAAACCTGTACTAATCTAA
NOV4k,
13379364 SNP
CG114555-01 SNP: Gly to Gly
Protein Sequence SEQ ID NO: 44 507 aa at position 28
MARKQNRNSKELGLVPLTDDTSHAGPPGPGRALLECDHLRSGVPGGRRRKYIKAFYNESWERRHGRPIDPDT
LTLLWSVTVSIFAIGGLVGTLIVKMIGKVLGRKHTLLANNGFAISAALLMACSLQAGAFEMLIVGRFIMGID
GGVALSVLPMYLSEISPKEIRGSLGQVTAIFICIGVFTGQLLGLPELLGKESTWPYLFGVIVVPAVVQLLSL
PFLPDSPRYLLLEKHNEARAVKAFQTFLGKADVSQEVEEVLAESRVQRSIRLVSVLELLRAPYVRWQVVTVI
VTMACYQLCGLNAIWFYTNSIFGKAGIPLAKIPYVTLSTGGIETLAAVFSGLVIEHLGRRPLLIGGFGLMGL
FFGTLTITLTLQDHAPWVPYLSIVGILAIIASFCSGPGGIPFILTGEFFQQSQRPAAFIIAGTVNWLSNFAV
GLLFPFIQKSLDTYCFLVFATI CITGAIYLYFVLPETKNRTYAEISQAFSKRNKAYPPEEKIDSAVTDGKIN
GRP
NOV4l,
13379363 SNP SEQ ID NO: 45
CG114555-01 ORF Start: ATG SNP: A/G at position 289
DNA Sequence at position 14 ORF Stop: TAA at 1535
GTCACTGAGACCCATGGCAAGGAAACAAAATAGGAATTCCAAGGAACTGGGCCTAGTTCCCCTCACAGATGA
CACCAGCCACGCCGGGCCTCCAGGGCCAGGGAGGGCACTGCTGGAGTGTGACCACCTGAGGAGTGGGGTGCC
AGGTGGAAGGAGAAGAAAGTACATCAAGGCCTTTTACAATGAGTCATGGGAAAGAAGGCATGGACGTCCAAT
AGACCCAGACACTCTGACTCTGCTCTGGTCTGTGACTGTGTCCATATTCGCCATCGGTGGACTTGTGGGGAC
GTTAATTGTGAAGATGATTGGAAAGGTTCTTGGGAGGAAGCACACTTTGCTGGCCAATAATGGGTTTGCAAT
TTCTGCTGCATTGCTGATGGCCTGCTCGCTCCAGGCAGGAGCCTTTGAAATGCTCATCGTGGGACGCTTCAT
CATGGGCATAGATGGAGGCGTCGCCCTCAGTGTGCTCCCCATGTACCTCAGTGAGATCTCACCCAAGGAGAT
CCGTGGCTCTCTGGGGCAGGTGACTGCCATCTTTATCTGCATTGGCGTGTTCACTGGGCAGCTTCTGGGCCT
GCCCGAGCTGCTGGGAAAGGAGAGTACCTGGCCATACCTGTTTGGAGTGATTGTGGTCCCTGCCGTTGTCCA
GCTGCTGAGCCTTCCCTTTCTCCCGGACAGCCCACGCTACCTGCTCTTGGAGAAGCACAACGAGGCAAGAGC
TGTGAAAGCCTTCCAAACGTTCTTGGGTAAAGCAGACGTTTCCCAAGAGGTAGAGGAGGTCCTGGCTGAGAG
CCGCGTGCAGAGGAGCATCCGCCTGGTGTCCGTGCTGGAGCTGCTGAGAGCTCCCTACGTCCGCTGGCAGGT
GGTCACCGTGATTGTCACCATGGCCTGCTACCAGCTCTGTGGCCTCAATGCAATTTGGTTCTATACCAACAG
CATCTTTGGAAAAGCTGGGATCCCTCTGGCAAAGATCCCATACGTCACCTTGAGTACAGGGGGCATCGAGAC
TTTGGCTGCCGTCTTCTCTGGTTTGGTCATTGAGCACCTGGGACGGAGACCCCTCCTCATTGGTGGCTTTGG
GCTCATGGGCCTCTTCTTTGGGACCCTCACCATCACGCTGACCCTGCAGGACCACGCCCCCTGGGTCCCCTA
CCTGAGTATCGTGGGCATTCTGGCCATCATCGCCTCTTTCTGCAGTGGGCCAGGTGGCATCCCGTTCATCTT
GACTGGTGAGTTCTTCCAGCAATCTCAGCGGCCGGCTGCCTTCATCATTGCAGGCACCGTCAACTGGCTCTC
CAACTTTGCTGTTGGGCTCCTCTTCCCATTCATTCAGAAAAGTCTGGACACCTACTGTTTCCTAGTCTTTGC
TACAATTTGTATCACAGGTGCTATCTACCTGTATTTTGTGCTGCCTGAGACCAAAAACAGAACCTATGCAGA
AATCAGCCAGGCATTTTCCAAAAGGAACAAAGCATACCCACCAGAAGAGAAAATCGACTCAGCTGTCACTGA
TGGTAAGATAAATGGAAGGCCTTAACAAGTTTCCTCCTCCACGTTGGACAATTATGTCAAAAACAGGATTGT
CTACATGGATGATCTCACTTTTCAGGAAACTTAAAATTTACCCATTATTGGGAAGCTTAAATGAATTGAAGC
TATGCAAGTCTTTTATATTATTAAATATTTAAAAGTAAACCTGTACTAATCTAA
NOV4l,
13379363 SNP
CG114555-01 SNP: no change in the
Protein Sequence SEQ ID NO: 46 507 aa protein sequence
MARKQNRNSKELGLVPLTDDTSHAGPPGPGRALLECDHLRSGVPGGRRRKYIKAFYNESWERRHGRPIDPDT
LTLLWSVTVSIFAIGGLVGTLIVKMIGKVLGRKHTLLANNGFAISAALLMACSLQAGAFEMLIVGRFIMGID
GGVALSVLPMYLSEISPKEIRGSLGQVTAIFICIGVFTGQLLGLPELLGKESTWPYLFGVIVVPAVVQLLSL
PFLPDSPRYLLLEKHNEARAVKAFQTFLGKADVSQEVEEVLAESRVQRSIRLVSVLELLRAPYVRWQVVTVI
VTMACYQLCGLNAIWFYTNSIFGKAGIPLAKIPYVTLSTGGIETLAAVFSGLVIEHLGRRPLLIGGFGLMGL
FFGTLTITLTLQDHAPWVPYLSIVGILAIIASFCSGPGGIPFILTGEFFQQSQRPAAFIIAGTVNWLSNFAV
GLLFPFIQKSLDTYCFLVFATICITGAIYLYFVLPETKNRTYAEISQAFSKRNKAYPPEEKIDSAVTDGKIN
GRP
NOV4m,
13379362 SNP SEQ ID NO: 47
CG114555-01 ORF Start: ATG SNP: C/T at position 672
DNA Sequence at position 14 ORF Stop: TAA at 1535
GTCACTGAGACCCATGGCAAGGAAACAAAATAGGAATTCCAAGGAACTGGGCCTAGTTCCCCTCACAGATGA
CACCAGCCACGCCGGGCCTCCAGGGCCAGGGAGGGCACTGCTGGAGTGTGACCACCTGAGGAGTGGGGTGCC
AGGTGGAAGGAGAAGAAAGTACATCAAGGCCTTTTACAATGAGTCATGGGAAAGAAGGCATGGACGTCCAAT
AGACCCAGACACTCTGACTCTGCTCTGGTCTGTGACTGTGTCCATATTCGCCATCGGTGGACTTGTGGGGAC
ATTAATTGTGAAGATGATTGGAAAGGTTCTTGGGAGGAAGCACACTTTGCTGGCCAATAATGGGTTTGCAAT
TTCTGCTGCATTGCTGATGGCCTGCTCGCTCCAGGCAGGAGCCTTTGAAATGCTCATCGTGGGACGCTTCAT
CATGGGCATAGATGGAGGCGTCGCCCTCAGTGTGCTCCCCATGTACCTCAGTGAGATCTCACCCAAGGAGAT
CCGTGGCTCTCTGGGGCAGGTGACTGCCATCTTTATCTGCATTGGCGTGTTCACTGGGCAGCTTCTGGGCCT
GCCCGAGCTGCTGGGAAAGGAGAGTACCTGGCCATACCTGTTTGGAGTGATTGTGGTCCCTGCCGTTGTCCA
GCTGCTGAGCCTTCCCTTTCTCCTGGACAGCCCACGCTACCTGCTCTTGGAGAAGCACAACGAGGCAAGAGC
TGTGAAAGCCTTCCAAACGTTCTTGGGTAAAGCAGACGTTTCCCAAGAGGTAGAGGAGGTCCTGGCTGAGAG
CCGCGTGCAGAGGAGCATCCGCCTGGTGTCCGTGCTGGAGCTGCTGAGAGCTCCCTACGTCCGCTGGCAGGT
GGTCACCGTGATTGTCACCATGGCCTGCTACCAGCTCTGTGGCCTCAATGCAATTTGGTTCTATACCAACAG
CATCTTTGGAAAAGCTGGGATCCCTCTGGCAAAGATCCCATACGTCACCTTGAGTACAGGGGGCATCGAGAC
TTTGGCTGCCGTCTTCTCTGGTTTGGTCATTGAGCACCTGGGACGGAGACCCCTCCTCATTGGTGGCTTTGG
GCTCATGGGCCTCTTCTTTGGGACCCTCACCATCACGCTGACCCTGCAGGACCACGCCCCCTGGGTCCCCTA
CCTGAGTATCGTGGGCATTCTGGCCATCATCGCCTCTTTCTGCAGTGGGCCAGGTGGCATCCCGTTCATCTT
GACTGGTGAGTTCTTCCAGCAATCTCAGCGGCCGGCTGCCTTCATCATTGCAGGCACCGTCAACTGGCTCTC
CAACTTTGCTGTTGGGCTCCTCTTCCCATTCATTCAGAAAAGTCTGGACACCTACTGTTTCCTAGTCTTTGC
TACAATTTGTATCACAGGTGCTATCTACCTGTATTTTGTGCTGCCTGAGACCAAAAACAGAACCTATGCAGA
AATCAGCCAGGCATTTTCCAAAAGGAACAAAGCATACCCACCAGAAGAGAAAATCGACTCAGCTGTCACTGA
TGGTAAGATAAATGGAAGGCCTTAACAAGTTTCCTCCTCCACGTTGGACAATTATGTCAAAAACAGGATTGT
CTACATGGATGATCTCACTTTTCAGGAAACTTAAAATTTACCCATTATTGGGAAGCTTAAATGAATTGAAGC
TATGCAAGTCTTTTATATTATTAAATATTTAAAAGTAAACCTGTACTAATCTAA
NOV4m, 13379362
SNP CG114555-01 SNP: Pro to Leu
Protein Sequence SEQ ID NO: 48 507 aa at position 220
MARKQNRNSKELGLVPLTDDTSHAGPPGPGRALLECDHLRSGVPGGRRRKYIKAFYNESWERRHGRPIDPD
TLTLLWSVTVSIFAIGGLVGTLIVKMIGKVLGRKHTLLANNGFAISAALLMACSLQAGAFEMLIVGRFIMG
IDGGVALSVLPMYLSEISPKEIRGSLGQVTAIFICIGVFTGQLLGLPELLGKESTWPYLFGVIVVPAVVQL
LSLPFLLDSPRYLLLEKHNEARAVKAFQTFLGKADVSQEVEEVLAESRVQRSIRLVSVLELLRAPYVRWQV
VTVIVTMACYQLCGLNAIWFYTNSIFGKAGIPLAKIPYVTLSTGGIETLAAVFSGLVIEHLGRRPLLIGGF
GLMGLFFGTLTITLTLQDHAPWVPYLSIVGILAIIASFCSGPGGIPFILTGEFFQQSQRPAAFIIAGTVNW
LSNFAVGLLFPFIQKSLDTYCFLVFATICITGAIYLYFVLPETKNRTYAEISQAFSKRNKAYPPEEKIDSA
VTDGKINGRP
NOV4n,
13379620 SNP SEQ ID NO: 49
CG114555-01 ORF Start: ATG SNP: T/C at position 963
DNA Sequence at position 14 ORF Stop: TAA at 1535
GTCACTGAGACCCATGGCAAGGAAACAAAATAGGAATTCCAAGGAACTGGGCCTAGTTCCCCTCACAGATG
ACACCAGCCACGCCGGGCCTCCAGGGCCAGGGAGGGCACTGCTGGAGTGTGACCACCTGAGGAGTGGGGTG
CCAGGTGGAAGGAGAAGAAAGTACATCAAGGCCTTTTACAATGAGTCATGGGAAAGAAGGCATGGACGTCC
AATAGACCCAGACACTCTGACTCTGCTCTGGTCTGTGACTGTGTCCATATTCGCCATCGGTGGACTTGTGG
GGACATTAATTGTGAAGATGATTGGAAAGGTTCTTGGGAGGAAGCACACTTTGCTGGCCAATAATGGGTTT
GCAATTTCTGCTGCATTGCTGATGGCCTGCTCGCTCCAGGCAGGAGCCTTTGAAATGCTCATCGTGGGACG
CTTCATCATGGGCATAGATGGAGGCGTCGCCCTCAGTGTGCTCCCCATGTACCTCAGTGAGATCTCACCCA
AGGAGATCCGTGGCTCTCTGGGGCAGGTGACTGCCATCTTTATCTGCATTGGCGTGTTCACTGGGCAGCTT
CTGGGCCTGCCCGAGCTGCTGGGAAAGGAGAGTACCTGGCCATACCTGTTTGGAGTGATTGTGGTCCCTGC
CGTTGTCCAGCTGCTGAGCCTTCCCTTTCTCCCGGACAGCCCACGCTACCTGCTCTTGGAGAAGCACAACG
AGGCAAGAGCTGTGAAAGCCTTCCAAACGTTCTTCGGTAAAGCAGACGTTTCCCAAGAGGTAGAGGAGGTC
CTGGCTGAGAGCCGCGTGCAGAGGAGCATCCGCCTGGTGTCCGTGCTGGAGCTGCTGAGAGCTCCCTACGT
CCGCTGGCAGGTGGTCACCGTGATTGTCACCATGGCCTGCTACCAGCTCTGTGGCCTCAATGCAATTTGGT
TCTATACCAACAGCATCTTTGGAAAAGCTGGGATCCCTCCGGCAAAGATCCCATACGTCACCTTGAGTACA
GGGGGCATCGAGACTTTGGCTGCCGTCTTCTCTGGTTTGGTCATTGAGCACCTGGGACGGAGACCCCTCCT
CATTGGTGGCTTTGGGCTCATGGGCCTCTTCTTTGGGACCCTCACCATCACGCTGACCCTGCAGGACCACG
CCCCCTGGGTCCCCTACCTGAGTATCGTGGGCATTCTGGCCATCATCGCCTCTTTCTGCAGTGGGCCAGGT
GGCATCCCGTTCATCTTGACTGGTGAGTTCTTCCAGCAATCTCAGCGGCCGGCTGCCTTCATCATTGCAGG
CACCGTCAACTGGCTCTCCAACTTTGCTGTTGGGCTCCTCTTCCCATTCATTCAGAAAAGTCTGGACACCT
ACTGTTTCCTAGTCTTTGCTACAATTTGTATCACAGGTGCTATCTACCTGTATTTTGTGCTGCCTGAGACC
AAAAACAGAACCTATGCAGAAATCAGCCAGGCATTTTCCAAAAGGAACAAAGCATACCCACCAGAAGAGAA
AATCGACTCAGCTGTCACTGATGGTAAGATAAATGGAAGGCCTTAACAAGTTTCCTCCTCCACGTTGGACA
ATTATGTCAAAAACAGGATTGTCTACATGGATGATCTCACTTTTCAGGAAACTTAAAATTTACCCATTATT
GGGAAGCTTAAATGAATTGAAGCTATGCAAGTCTTTTATATTATTAAATATTTAAAAGTAAACCTGTACTA
ATCTAA
NOV4n,
13379620 SNP
CG114555-01 SNP: Leu to Pro
Protein Sequence SEQ ID NO: 50 507 aa at position 317
MARKQNRNSKELGLVPLTDDTSHAGPPGPGRALLECDHLRSGVPGGRRRKYIKAFYNESWE
RRHGRPIDPDTLTLLWSVTVSIFAIGGLVGTLIVKMIGKVLGRKHTLLANNGFAISAALLM
ACSLQAGAFEMLIVGRFIMGIDGGVALSVLPMYLSEISPKEIRGSLGQVTAIFICIGVFTG
QLLGLPELLGKESTWPYLFGVIVVPAVVQLLSLPFLPDSPRYLLLEKHNEARAVKAFQTFL
GKADVSQEVEEVLAESRVQRSIRLVSVLELLRAPYVRWQVVTVIVTMACYQLCGLNAIWFY
TNSIFGKAGIPPAKIPYVTLSTGGIETLAAVFSGLVIEHLGRRPLLIGGFGLMGLFFGTLT
ITLTLQDHAPWVPYLSIVGILAIIASFCSGPGGIPFILTGEFFQQSQRPAAFIIAGTVNWL
SNFAVGLLFPFIQKSLDTYCFLVFATICITGAIYLYFVLPETKNRTYAEISQAFSKRNKAY
PPEEKIDSAVTDGKINGRP

A ClustalW comparison of the above protein sequences yields the following sequence alignment shown in Table 4B.

TABLE 4B
Comparison of the NOV4 protein sequences.

| | |
|---|---|
| NOV4a | MARKQNRNSKELGLVPLTDDTSHAGPPGPGRALLECDHLRSGVPGGRRRK---------- |
| NOV4b | ------------------------------------------------------------ |
| NOV4c | ------------------------------------------------------------ |
| NOV4d | ------------------------------------------------------------ |
| NOV4e | ------------------------------------------------------------ |
| NOV4f | ------------------------------------------------------------ |
| NOV4g | MARKQNRNSKELGLVPLTDDTSHARPPGPGRALLECVHLRSGVPGGRRRKDW-------- |
| NOV4h | MARKQNRNSKELGLVPLTDDTSHAGPPGPGRALLECDHLRSGVPGGRRRKQPLRSTSSAA |
| NOV4i | MARKQNRNSKELGLVPLTDDTSHARPPGPGRALLECVHLRSGVPGGRRRKDW-------- |
| NOV4a | ------------------------------------------------YIKAFYNESWER |
| NOV4b | ------------------------------------------------------------ |
| NOV4c | ------------------------------------------------------------ |
| NOV4d | ------------------------------------------------------------ |
| NOV4e | ------------------------------------------------------------ |
| NOV4f | ------------------------------------------------------------ |
| NOV4g | --------SCSLLV-----ASLAGAFGSPFLYGYNLS----VVNAPTPYIKAFYNESWER |
| NOV4h | GSSTTYVASAAIKIPFHRWASLAPEKSSHLLRLLQLRGLALEVKLISLGSKPQSSISWLV |
| NOV4i | --------SCSLLV-----ASLAGAFGSPFLYGYNLS----VVNAPTPYIKAFYNESWER |
| NOV4a | RHGRPIDPDTLTLLWSVTVSIFAIGGLVGTLIVKMIGKVLGRKHTLLANNGFAISAALLM |
| NOV4b | --------------------------LYKKAGSAAAPFTGTRKHTLLANNGFAISAALLM |
| NOV4c | -------------------------------GSAAAPFTGTRKHTLLANNGFAISAALLM |
| NOV4d | -------------------------------GSAAAPFTGTRKHTLLANNGFAISAALLM |
| NOV4e | -------------------------------GSAAAPFTGTRKHTLLANNGFAISAALLM |
| N0V4f | -------------------------------GSAAAPFTGTRKHTLLANNGFAISAALLM |
| NOV4g | RHGRPIDPDTLTLLWSVTVSIFAIGGLVGTLIVKMIGKVLGRKHTLLANNGFAISAALLM |
| NOV4h | ALEETNEIGRDWSCSLLVASLAGAFGSSFLYGYNLSVVNAPTPHTLLANNGFAISAALLM |
| NOV4i | RHGRPIDPDTLTLLWSVTVSIFAIGGLVGTLIVKMIGKVLGRKHTLLANNGFAISAALLM |
| NOV4a | ACSLQAGAFEMLIVGRFIMGIDGGVALSVLPMYLSEISPKEIRGSLGQVTAIFICIGVFT |
| NOV4b | ACSLQAGAFEMLIVGRFIMGIDGGVALSVLPMYLSEISPKEIRGSLGQVTAIFICIGVFT |
| NOV4c | ACSLQAGAFEMLIVGRFIMGIDGGVALSVLPMYLSEISPKEIRGSLGQVTAIFICIGVFT |
| NOV4d | ACSLQAGAFEMLIVGRFIMGIDGGVALSVLPMYLSEISPKEIRGSLGQVTAIFICIGVFT |
| NOV4e | ACSLQAGAFEMLIVGRFIMGIDGGVALSVLPMYLSEISPKEIRGSLGQVTAIFICIGVFT |
| NOV4f | ACSLQAGAFEMLIVGRFIMGIDGGVALSVLPMYLSEISPKEIRGSLGQVTAIFICIGVFT |
| NOV4g | ACSLQAGAFEMLIVGRFIMGIDGGVALSVLPMYLSEISPKEIRGSLGQVTAIFICIGVFT |
| NOV4h | ACSLQAGAFEMLIVGRFIMGIDGGVALSVLPMYLSEISPKEIRGSLGQVTAIFICIGVFT |
| NOV4i | ACSLQAGAFEMLIVGRFIMGIDGGVALSVLPMYLSEISPKEIRGSLGQVTAIFICIGVFT |
| NOV4a | GQLLGLPELLGKESTWPYLFGVIVVPAVVQLLSLPFLPDSPRYLLLEKHNEARAVKAFQT |
| NOV4b | GQLLGLPELLGKESTWPYLFGVIVVPAVVQLLSLPFLPDSPRYLLLEKHNEARAVKAFQT |
| NOV4c | GQLLGLPELLGKESTWPYLFGVIVVPAVVQLLSLPFLPDSPRYLLLEKHNEARAVKAFQT |
| NOV4d | GQLLGLPELLGKESTWPYLFGVIVVPAVVQLLSLPFLPDSPRYLLLEKHNEARAVKAFQT |
| NOV4e | GQLLGLPELLGKESTWPYLFGVIVVPAVVQLLSLPFLPDSPRYLLLEKHNEARAVKAFQT |
| NOV4f | GQLLGLPELLGKESTWPYLFGVIVVPAVVQLLSLPFLPDSPRYLLLEKHNEARAVKAFQT |
| NOV4g | GQLLGLPELLGKESTWPYLFGVIVVPAVVQLLSLPFLLDSPRYLLLEKHNEARAVKAFQT |
| NOV4h | GQLLGLPELLGKESTWPYLFGVIVVPAVVQLLSLPFLLDSPRYLLLEKHNEARAVKAFQT |
| NOV4i | GQLLGLPELLGKESTWPYLFGVIVVPAVVQLLSLPFLLDSPRYLLLEKHNEARAVKAFQT |
| NOV4a | FLGKADVSQEVEEVLAESRVQRSIRLVSVLELLRAPYVRWQVVTVIVTMACYQLCGLNAI |
| NOV4b | FLGKADVSQEVEEVLAESRVQRSIRLVSVLELLRAPYVRWQVVTVIVTMACYQLCGLNAI |
| NOV4c | FLGKADVSQEVEEVLAESRVQRSIRLVSVLELLRAPYVRWQVVTVIVTMACYQLCGLNAI |
| NOV4d | FLGKADVSQEVEEVLAESRVQRSIRLVSVLELLRAPYVRWQVVTVIVTMACYQLCGLNAI |
| NOV4e | FLGKADVSQEVEEVLAESRVQRSIRLVSVLELLRAPYVRWQVVTVIVTMACYQLCGLNAI |
| NOV4f | FLGKADVSQEVEEVLAESRVQRSIRLVSVLELLRAPYVRWQVVTVIVTMACYQLCGLNAI |
| NOV4g | FLGKADVSQEVEEVLAESRVQRSIRLVSVLELLRAPYVRWQVVTVIVTMACYQLCGLNAI |
| NOV4h | FLGKADVSQEVEEVLAESRVQRSIRLVSVLELLRAPYVRWQVVTVIVTMACYQLCGLNAI |
| NOV4i | FLGKADVSQEVEEVLAESRVQRSIRLVSVLELLRAPYVRWQVVTVIVTMACYQLCGLNAI |
| NOV4a | WFYTNSIFGKAGIPLAKIPYVTLSTGGIETLAAVFSGLVIEHLGRRPLLIGGFGLMGLFF |
| NOV4b | WFYTNSIFGKAGIPPAKIPYVTLSTGGIETLAAVFSGLVIEHLGRRPLLIGGFGLMGLFF |
| NOV4c | WFYTNSIFGKAGIPPAKIPYVTLSTGGIETLAAVFS------------------------ |
| NOV4d | WFYTNSIFGKAGIPPAKIPYVTLSTGGIETLAAVFSGLVIEHLGRRPLLIGGFGLMGLFF |
| NOV4e | WFYTNSIFGKAGIPPAKIPYVTLSTGGIETLAAVFSGLVIEHLGRRPLLIGGFGLMGLFF |
| NOV4f | WFYTNSIFGKAGIPLAKIPYVTLSTGGIETLAAVFSGLVIEHLGRRPLLIGGFGLMGLFF |
| NOV4g | WFYTNSIFGKAGIPLAKIPYVTLSTGGIETLAAVFSGLVIEHLGRRPLLIGGFGLMGLFF |
| NOV4h | WFYTNSIFGKAGIPLAKIPYVTLSTGGIETLAAVFSGLVIEHLGRRPLLIGGFGLMGLFF |
| NOV4i | WFYTNSIFGKAGIPLAKIPYVTLSTGGIETLAAVFS------------------------ |
| NOV4a | GTLTITLTLQDHAPWVPYLSIVGILAIIASFCSGPGGIPFILTGEFFQQSQRPAAFIIAG |
| NOV4b | GTLTITLTLQDHAPWVPYLSIVGILAIIASFCSGPGGIPFILTGEFFQQSQRPAAFIIAG |
| NOV4c | ----------DHAPWVPYLSIVGILAIIASFCSGPGGIPFILTGEFFQQSQRPAAFIIAG |
| NOV4d | GTLTITLTLQDHAPWVPYLSIVGILAIIASFCSGPGGIPFILTGEFFQQSQRPAAFIIAG |
| NOV4e | GTLTITLTLQDHAPWVPYLSIVGILAIIASFCSGPGGIPFILTGEFFQQSQRPAAFIIAG |
| NOV4f | GTLTITLTLQDHAPWVPYLSIVGILAIIASFCSGPGGIPFILTGEFFQQSQRPAAFIIAG |
| NOV4g | GALTITLTLQDHAPWVPYLSIVGILAIIASFCSGPGGIPFILTGEFFQQSQRPAAFIIAG |
| NOV4h | GALTITLTLQDHAPWVPYLSIVGILAIIASFCSGPGGIPFILTGEFFQQSQRPAAFIIAG |
| NOV4i | ------------------------------------GIPFILTGEFFQQSQRPAAFIIAG |
| NOV4a | TVNWLSNFAVGLLFPFIQKSLDTYCFLVFATICITGAIYLYFVLPETKNRTYAEISQAFS |
| NOV4b | TVNWLSNFAVGLLFPFIQKSLDTYCFLVFATICITGAIYLYFVLPETKNRTYAEISQAFL |
| NOV4c | TVNWLSNFAVGLLFPFIQKSLDTYCFLVFATICITGAIYLYFVLPETKNRTYAEISQAFL |
| NOV4d | TVNWLSNFAVGLLFPFIQKSLDTYCFLVFATICITGAIYLYFVLPETKNRTYAEISQAFL |
| NOV4e | TVNWLSNFAVGLLFPFIQKSLDTYCFLVFATICITGAIYLYFVLPETKNRTYAEISQAFL |
| NOV4f | TVNWLSNFAVGLLFPFIQKSLDTYCFLVFATICITGAIYLYFVLPETKNRTYAEISQAFL |
| NOV4g | TVNWLSNFAVGLLFPFIQKSLDTYCFLVFATICITGAIYLYFVLPETKNRTYAEISQAFS |
| NOV4h | TVNWLSNFAVGLLFPFIQKSLDTYCFLVFATICITGAIYLYFVLPETKNRTYAEISQAFS |
| NOV4i | TVNWLSNFAVGLLFPFIQKSLDTYCFLVFATICITGAIYLYFVLPETKNRTYAEISQAFS |
| NOV4a | KRNKAYPPEEKIDSAVTDGKINGRP |
| NOV4b | E-----------------GKG-GRA |
| NOV4c | E-----------------GKG-GRA |
| NOV4d | E-----------------GKG-GRA |
| NOV4e | E-----------------GKG-GRA |
| NOV4f | E-----------------GKG-GRA |
| NOV4g | KRNKAYPPEEKIDSAVTDGKINGRP |
| NOV4h | KRNKAYPPEEKIDSAVTDGKINGRP |
| NOV4i | KRNKAYPPEEKIDSAVTDGKINGRP |
| NOV4a | (SEQ ID NO: 24) |
| NOV4b | (SEQ ID NO: 26) |
| NOV4c | (SEQ ID NO: 28) |
| NOV4d | (SEQ ID NO: 30) |
| NOV4e | (SEQ ID NO: 32) |
| NOV4f | (SEQ ID NO: 34) |
| NOV4g | (SEQ ID NO: 33) |
| NOV4h | (SEQ ID NO: 38) |
| NOV4i | (SEQ ID NO: 40) |

Further analysis of the NOV4a protein yielded the following properties shown in Table 4C.

TABLE 4C
Protein Sequence Properties NOV4a

| SignalP | No Known Signal Sequence Predicted | | | | |
|---|---|---|---|---|---|
| analysis: | | | | | |
| PSORT II | PSG: a new signal peptide prediction method | | | | |
| analysis: | N-region: length 11; pos. chg 4; neg. chg 1 | | | | |
| | H-region: length 7; peak value 1.99 | | | | |
| | PSG score: −2.41 | | | | |
| | GvH: von Heijne's method for signal seq. recognition | | | | |
| | GvH score (threshold: −2.1): −4.97 | | | | |
| | possible cleavage site: between 24 and 25 | | | | |
| | >>> Seems to have no N-terminal signal peptide | | | | |
| | ALOM: Klein et al's method for TM region allocation | | | | |
| | Init position for calculation: 1 | | | | |
| | Tentative number of TMS(s) for the threshold 0.5: 9 | | | | |
| | INTEGRAL | Likelihood = | −6.48 | Transmembrane | 79-95 |
| | INTEGRAL | Likelihood = | −1.75 | Transmembrane | 120-136 |
| | INTEGRAL | Likelihood = | 0.47 | Transmembrane | 140-156 |
| | INTEGRAL | Likelihood = | −3.40 | Transmembrane | 171-187 |
| | INTEGRAL | Likelihood = | −5.73 | Transmembrane | 200-216 |
| | INTEGRAL | Likelihood = | −0.32 | Transmembrane | 283-299 |
| | INTEGRAL | Likelihood = | −2.23 | Transmembrane | 351-367 |
| | INTEGRAL | Likelihood = | −5.89 | Transmembrane | 378-394 |
| | INTEGRAL | Likelihood = | −5.26 | Transmembrane | 449-465 |

| | | |
|---|---|---|
| PERIPHERAL | Likelihood = | 1.01 (at 419) |

| ALOM score: −6.48 (number of TMSs: 9) | | | |
|---|---|---|---|
| MTOP: Prediction of membrane topology (Hartmann et al.) | | | |
| Center position for calculation: 86 | | | |
| Charge difference: 5.0 C(4.5)-N(−0.5) | | | |
| C > N: C-terminal side will be inside | | | |
| >>> membrane topology: type 3b | | | |
| MITDISC: discrimination of mitochondrial targeting seq | | | |
| R content: | 2 | Hyd Moment(75): | 10.97 |
| Hyd Moment(95): | 13.25 | G content: | 1 |
| D/E content: | 2 | S/T content: | 2 |

Score: −3.59
Gavel: prediction of cleavage sites for mitochondrial preseq
R-2 motif at 17 NRN|SK
NUCDISC: discrimination of nuclear localization signals
pat4: RRRK (5) at 47
pat7: PGGRRRK (5) at 44
bipartite: none
content of basic residues: 9.1%
NLS Score: 0.27
KDEL: ER retention motif in the C-terminus: none
ER Membrane Retention Signals:
XXRR-like motif in the N-terminus: ARKQ
none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 94.1
COIL: Lupas's algorithm to detect coiled-coil regions
total: 0 residues
Final Results (k = 9/23):
66.7%: endoplasmic reticulum
11.1%: vacuolar
11.1%: mitochondrial
11.1%: Golgi
>> prediction for CG114555-01 is end (k = 9)

A search of the NOV4a protein against the Geneseq database, a proprietary database that contains sequences published in patents and patent publication, yielded several homologous proteins shown in Table 4D.

TABLE 4D
Geneseq Results for NOV4a

| | | NOV4a | Identities/ | |
|---|---|---|---|---|
| | | Residues/ | Similarities for | |
| Geneseq | Protein/Organism/Length [Patent | Match | the Matched | Expect |
| Identifier | #, Date] | Residues | Region | Value |
| AAM79422 | Human protein SEQ ID NO 3068 - | 1 . . . 507 | 506/540 (93%) | 0.0 |
| | Homo sapiens, 558 aa. | 19 . . . 558 | 506/540 (93%) | |
| | [WO200157190-A2, 09 AUG. 2001] | | | |
| ABB11910 | Human GLUT9 homologue, SEQ ID | 1 . . . 507 | 506/540 (93%) | 0.0 |
| | NO: 2280 - Homo sapiens, 558 aa. | 19 . . . 558 | 506/540 (93%) | |
| | [WO200157188-A2, 09 AUG. 2001] | | | |
| AAM41316 | Human polypeptide SEQ ID NO | 1 . . . 507 | 505/540 (93%) | 0.0 |
| | 6247 - Homo sapiens, 558 aa. | 19 . . . 558 | 505/540 (93%) | |
| | [WO200153312-A1, 26 JUL. 2001] | | | |
| AAE16788 | Human transporter and ion channel- | 1 . . . 504 | 500/537 (93%) | 0.0 |
| | 25 (TRICH-25) protein - Homo | 1 . . . 537 | 501/537 (93%) | |
| | sapiens, 537 aa. [WO200192304-A2, | | | |
| | 06 DEC. 2001] | | | |
| AAE14611 | Human glucose transporter protein | 1 . . . 500 | 498/533 (93%) | 0.0 |
| | GLUTX - Homo sapiens, 563 aa. | 1 . . . 533 | 498/533 (93%) | |
| | [US6346374-B1, 12 FEB. 2002] | | | |

In a BLAST search of public sequence databases, the NOV4a protein was found to have homology to the proteins shown in the BLASTP data in Table 4E.

TABLE 4E
Public BLASTP Results for NOV4a

| | | NOV4a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9NRM0 | Solute carrier family 2, facilitated | 1 . . . 507 | 506/540 (93%) | 0.0 |
| | glucose transporter, member 9 | 1 . . . 540 | 506/540 (93%) | |
| | (Glucose transporter type 9) - Homo | | | |
| | sapiens (Human), 540 aa. | | | |
| Q8WV30 | Similar to solute carrier family 2 | 51 . . . 507 | 457/457 (100%) | 0.0 |
| | (Facilitated glucose transporter), | 55 . . . 511 | 457/457 (100%) | |
| | member 9 - Homo sapiens (Human), | | | |
| | 511 aa. | | | |
| P22732 | Solute carrier family 2, facilitated | 52 . . . 494 | 202/446 (45%) | e−112 |
| | glucose transporter, member 5 | 46 . . . 491 | 291/446 (64%) | |
| | (Glucose transporter type 5, small | | | |
| | intestine) (Fructose transporter) - | | | |
| | Homo sapiens (Human), 501 aa. | | | |
| G02864 | fructose transporter - human, 481 aa. | 52 . . . 494 | 201/446 (45%) | e−111 |
| | | 26 . . . 471 | 290/446 (64%) | |
| Q8R1N7 | Similar to solute carrier family 2 | 50 . . . 493 | 201/447 (44%) | e−111 |
| | (Facilitated glucose transporter), | 43 . . . 489 | 290/447 (63%) | |
| | member 5 - Mus musculus (Mouse), | | | |
| | 501 aa. | | | |

PFam analysis predicts that the NOV4a protein contains the domains shown in the Table 4F.

TABLE 4F
Domain Analysis of NOV4a

| | | Identities/ | |
|---|---|---|---|
| | NOV4a | Similarities | |
| Pfam | Match | for the | Expect |
| Domain | Region | Matched Region | Value |
| sugar_tr | 33 . . . 481 | 150/488 (31%) | 9.1e−95 |
| | | 332/488 (68%) | |

### Example 5

The NOV5 clone was analyzed, and the nucleotide and encoded polypeptide sequences are shown in Table 5A.

TABLE 5A
NOV5 Sequence Analysis

NOV5a,
CG181662-01 SEQ ID NO: 51 1492 bp
DNA Sequence ORF Start: ATG at 4 ORF Stop: TAA at 940
GAGATGGCGGCCACCGAGGGGGTCGGGGAGGCTGCGCAAGGGGGCGAGCCCGGGCAGCCGGCGCAACC
CCCGCCCCAGCCGCACCCACCGCCGCCCCAGCAGCAGCACAAGGAAGAGATGGCGGCCGAGGCTGGGG
AAGCCGTGGCGTCCCCCATGGACGACGGGTTTGTGAGCCTGGACTCGCCCTCCTATGTCCTATACAGG
CATTTCCGGAGAGTTCTTTTGAAGTCACTTCAGAAGGATCTACATGAGGAAATGAACTACATCACTGC
AATAATTGAGGAGCAGCCCAAAAACTATCAAGTTTGGCATCATAGGCGAGTATTAGTGGAATGGCTAA
GAGATCCATCTCAGGAGCTTGAATTTATTGCTGATATTCTTAATCAGGATGCAAAGAATTATCATGCC
TGGCAGCATCGACAATGGGTTATTCAGGAATTTAAACTTTGGGATAATGAGCTGCAGTATGTGGACCA
ACTTCTGAAAGAGGATGTGAGAAATAACTCTGTCTGGAACCAAAGATACTTCGTTATTTCTAACACCA
CTGGCTACAATGATCGTGCTGTATTGGAGAGAGAAGTCCAATACACTCTGGAAATGATTAAACTAGTA
CCACATAATGAAAGTGCATGGAACTATTTGAAAGGGATTTTGCAGGATCGTGGTCTTTCCAAATATCC
TAATCTGTTAAATCAATTACTTGATTTACAACCAAGTCATAGTTCCCCCTACCTAATTGCCTTTCTTG
TGGATATCTATGAAGACATGCTAGAAAATCAGTGTGACAATAAGGAAGACATTCTTAATAAAGCATTA
GAGTTATGTGAAATCCTAGCTAAAGAAAAGGACACTATAAGAAAGGAATATTGGAGATACATTGGAAG
ATCCCTTCAAAGCAAACACAGCACAGAAAATGACTCACCAACAAATGTACAGCAATAACACCATCCAG
AAGAACTTGATGGAATGCTTTTATTTTTTATTAAGGGACCCTGCAGGAGTTTCACACGAGAGTGGTCC
TTCCCTTTGCCTGTGGTGTAAAAGTGCATCACACAGGTATTGCTTTTTAACAAGAACTGATGCTCCTT
GGGTGCTGCTGCTACTCAGACTAGCTCTAAGTAATGTGATTCTTCTAAAGCAAAGTCATTGGATGGGA
GGAGGAAGAAAAAGTCCCATAAAGGAACTTTTGTAGTCTTATCAACATATAATCTAATCCCTTAGCAT
CAGCTCCTCCCTCAGTGGTACATGCGTCAAGATTTGTAGCAGTAATAACTGCAGGTCACTTGTATGTA
ATGGATGTGAGGTAGCCGAAGTTTGGTTCAGTAAGCAGGGAATACAGTCGTTCCATCAGAGCTGGTCT
GCACACTCACATTATCTTGCTATCACTGTAACCAACTAATGCCAAAAGAACGGTTTTGTAATAAAATT
ATAGCTGTATCTAAAAAAAAAAAAAAAAAAAAAAAAAAAAAAAAAAAAAAAAAAAAAAAACAAA
NOV5a,
CG181662-01
Protein Sequence SEQ ID NO: 52 312 aa MW at 36492.6 kD
MAATEGVGEAAQGGEPGQPAQPPPQPHPPPPQQQHKEEMAAEAGEAVASPMDDGFVSLDSPSYVLYRH
FRRVLLKSLQKDLHEEMNYITAIIEEQPKNYQVWHHRRVLVEWLRDPSQELEFIADILNQDAKNYHAW
QHRQWVIQEFKLWDNELQYVDQLLKEDVRNNSVWNQRYFVISNTTGYNDRAVLEREVQYTLEMIKLVP
HNESAWNYLKGILQDRGLSKYPNLLNQLLDLQPSHSSPYLIAFLVDIYEDMLENQCDNKEDILNKALE
LCEILAKEKDTIRKEYWRYIGRSLQSKHSTENDSPTNVQQ
NOV5b,
CG181662-02 SEQ ID NO: 53 1487 bp
DNA Sequence ORF Start: ATG at 17 ORF Stop: TAA at 953
CGGCCGCGTCGACGAGATGGCGGCCACCGAGGGGGTCGGGGAGGCTGCGCAAGGGGGCGAGCCCGGGC
AGCCGGCGCAACCCCCGCCCCAGCCGCACCCACCGCCGCCCCAGCAGCAGCACAAGGAAGAGATGGCG
GCCGAGGCTGGGGAAGCCGTGGCGTCCCCCATGGACGACGGGTTTGTGAGCCTGGACTCGCCCTCCTA
TGTCCTATACAGGCATTTCCGGAGAGTTCTTTTGAAGTCACTTCAGAAGGATCTACATGAGGAAATGA
ACTACATCACTGCAATAATTGAGGAGCAGCCCAAAAACTATCAAGTTTGGCATCATAGGCGAGTATTA
GTGGAATGGCTAAGAGATCCATCTCAGGAGCTTGAATTTATTGCTGATATTCTTAATCAGGATGCAAA
GAATTATCATGCCTGGCAGCATCGACAATGGGTTATTCAGGAATTTAAACTTTGGGATAATGAGCTGC
AGTATGTGGACCAACTTCTGAAAGAGGATGTGAGAAATAACTCTGTCTGGAACCAAAGATACTTCGTT
ATTTCTAACACCACTGGCTACAATGATCGTGCTGTATTGGAGAGAGAAGTCCAATACACTCTGGAAAT
GATTAAACTAGTACCACATAATGAAAGTGCATGGAACTATTTGAAAGGGATTTTGCAGGATCGTGGTC
TTTCCAAATATCCTAATCTGTTAAATCAATTACTTGATTTACAACCAAGTCATAGTTCCCCCTACCTA
ATTGCCTTTCTTGTGGATATCTATGAAGACATGCTAGAAAATCAGTGTGACAATAAGGAAGACATTCT
TAATAAAGCATTAGAGTTATGTGAAATCCTAGCTAAAGAAAAGGACACTATAAGAAAGGAATATTGGA
GATACATTGGAAGATCCCTTCAAAGCAAACACAGCACAGAAAATGACTCACCAACAAATGTACAGCAA
TAACACCATCCAGAAGAACTTGATGGAATGCTTTTATTTTTTATTAAGGGACCCTGCAGGAGTTTCAC
ACGAGAGTGGTCCTTCCCTTTGCCTGTGGTGTAAAAGTGCATCACACAGGTATTGCTTTTTAACAAGA
ACTGATGCTCCTTGGGTGCTGCTGCTACTCAGACTAGCTCTAAGTAATGTGATTCTTCTAAAGCAAAG
TCATTGGATGGGAGGAGGAAGAAAAAGTCCCATAAAGGAACTTTTGTAGTCTTATCAACATATAATCT
AATCCCTTAGCATCAGCTCCTCCCTCAGTGGTACATGCGTCAAGATTTGTAGCAGTAATAACTGCACG
TCACTTGTATGTAATGGATGTGAGGTAGCCGAAGTTTGGTTCAGTAAGCAGGGAATACAGTCGTTCCA
TCAGAGCTGGTCTGCACACTCACATTATCTTGCTATCACTGTAACCAACTAATGCCAAAAGAACGGTT
TTGTAATAAAATTATAGCTGTATCTAAAAACAAAAAAAAAAAAAAAAAACCAAAAAAAT
NOV5b,
CG181662-02
Protein Sequence SEQ ID NO: 54 312 aa MW at 36492.6 kD
MAATEGVGEAAQGGEPGQPAQPPPQPHPPPPQQQHKEEMAAEAGEAVASPMDDGFVSLDSPSYVLYRH
FRRVLLKSLQKDLHEEMNYITAIIEEQPKNYQVWHHRRVLVEWLRDPSQELEFIADILNQDAKNYHAW
QHRQWVIQEFKLWDNELQYVDQLLKEDVRNNSVWNQRYFVISNTTGYNDRAVLEREVQYTLEMIKLVP
HNESAWNYLKGILQDRGLSKYPNLLNQLLDLQPSHSSPYLIAFLVDIYEDMLENQCDNKEDILNKALE
LCEILAKEKDTIRKEYWRYIGRSLQSKHSTENDSPTNVQQ
NOV5c,
307686795 SEQ ID NO: 55 1487 bp
DNA Sequence ORF Start: at 2 ORF Stop: TAA at 953
CGGCCGCGTCGACGAGATGGCGGCCACCGAGGGGGTCGGGGAGGCTGCGCAAGGGGGCGAGCCCGGGC
AGCCGGCGCAACCCCCGCCCCAGCCGCACCCACCGCCGCCCCAGCAGCAGCACAAGGAAGAGATGGCG
GCCGAGGCTGGGGAAGCCGTGGCGTCCCCCATGGACGACGGGTTTGTGAGCCTGGACTCGCCCTCCTA
TGTCCTATACAGGCATTTCCGGAGAGTTCTTTTGAAGTCACTTCAGAAGGATCTACATGAGGAAATGA
ACTACATCACTGCAATAATTGAGGAGCAGCCCAAAAACTATCAAGTTTGGCATCATAGGCGAGTATTA
GTGGAATGGCTAAGAGATCCATCTCAGGAGCTTGAATTTATTGCTGATATTCTTAATCAGGATGCAAA
GAATTATCATGCCTGGCAGCATCGACAATGGGTTATTCAGGAATTTAAACTTTGGGATAATGAGCTGC
AGTATGTGGACCAACTTCTGAAAGAGGATGTGAGAAATAACTCTGTCTGGAACCAAAGATACTTCGTT
ATTTCTAACACCACTGGCTACAATGATCGTGCTGTATTGGAGAGAGAAGTCCAATACACTCTGGAAAT
GATTAAACTAGTACCACATAATGAAAGTGCATGGAACTATTTGAAAGGGATTTTGCAGGATCGTGGTC
TTTCCAAATATCCTAATCTGTTAAATCAATTACTTGATTTACAACCAAGTCATAGTTCCCCCTACCTA
ATTGCCTTTCTTGTGGATATCTATGAAGACATGCTAGAAAATCAGTGTGACAATAAGGAAGACATTCT
TAATAAAGCATTAGAGTTATGTGAAATCCTAGCTAAAGAAAAGGACACTATAAGAAAGGAATATTGGA
GATACATTGGAAGATCCCTTCAAAGCAAACACAGCACAGAAAATGACTCACCAACAAATGTACAGCAA
TAACACCATCCAGAAGAACTTGATGGAATGCTTTTATTTTTTATTAAGGGACCCTGCAGGAGTTTCAC
ACGAGAGTGGTCCTTCCCTTTGCCTGTGGTGTAAAAGTGCATCACACAGGTATTGCTTTTTAACAAGA
ACTGATGCTCCTTGGGTGCTGCTGCTACTCAGACTAGCTCTAAGTAATGTGATTCTTCTAAAGCAAAG
TCATTGGATGGGAGGAGGAAGAAAAAGTCCCATAAAGGAACTTTTGTAGTCTTATCAACATATAATCT
AATCCCTTAGCATCAGCTCCTCCCTCAGTGGTACATGCGTCAAGATTTGTAGCAGTAATAACTGCAGG
TCACTTGTATGTAATGGATGTGAGGTAGCCGAAGTTTGGTTCAGTAAGCAGGGAATACAGTCGTTCCA
TCAGAGCTGGTCTGCACACTCACATTATCTTGCTATCACTGTAACCAACTAATGCCAAAAGAACGGTT
TTGTAATAAAATTATAGCTGTATCTAAAAACAAAAAAAAAAAAAAAAAACCAAAAAAAT
NOV5c,
307686795
Protein Sequence SEQ ID NO: 56 317 aa MW at 37049.2 kD
GRVDEMAATEGVGEAAQGGEPGQPAQPPPQPHPPPPQQQHKEEMAAEAGEAVASPMDDGFVSLDSPSY
VLYRHFRRVLLKSLQKDLHEEMNYITAIIEEQPKNYQVWHHRRVLVEWLRDPSQELEFIADILNQDAK
NYHAWQHRQWVIQEFKLWDNELQYVDQLLKEDVRNNSVWNQRYFVISNTTGYNDRAVLEREVQYTLEM
IKLVPHNESAWNYLKGILQDRGLSKYPNLLNQLLDLQPSHSSPYLIAFLVDIYEDMLENQCDNKEDIL
NKALELCEILAKEKDTIRKEYWRYIGRSLQSKHSTENDSPTNVQQ
NOV5d,
CG181662-03 SEQ ID NO: 57 1344 bp
DNA Sequence ORF Start: ATG at 17 ORF Stop: TAA at 1154
TCGGTCCGCAGCCGAGATGCCGGCCACCGAGGGGGTCGGGGAGGCTGCGCAAGGGGGCGAGCCCGGGC
AGCCGGCGCAACCCCCGCCCCAGCCGCACCCACCGCCGCCCCAGCAGCAGCACAAGGAAGAGATGGCG
GCCGAGGCTGGGGAAGCCGTGGCGTCCCCCATGGACGACGGGTTTGTGAGCCTGGACTCGCCCTCCTA
TGTCCTGTACAGGGACAGAGCAGAATGGGCTGATATAGATCCGGTGCCGCAGAATGATGGCCCCAATC
CCGTGGTCCAGATCATTTATAGTGACAAATTTAGAGATGTTTATGATTACTTCCGAGCTGTCCTGCAG
CGTGATGAAAGAAGTGAACGAGCTTTTAAGCTAACCCGGGATGCTATTGAGTTAAATGCAGCCAATTA
TACAGTGTGGCATTTCCGGAGAGTTCTTTTGAAGTCACTTCAGAAGGATCTACATGAGGAAATGAACT
ACATCACTGCAATAATTGAGGAGCAGCCCAAAAACTATCAAGTTTGGCATCATAGGCGAGTATTAGTG
GAATGGCTAAGAGATCCATCTCAGGAGCTTGAATTTATTGCTGATATTCTTAATCAGGATGCAAAGAA
TTATCATGCCTGGCAGCATCGACAATGGGTTATTCAGGAATTTAAACTTTGGGATAATGAGCTGCAGT
ATGTGGACCAACTTCTGAAAGAGGATGTGAGAAATAACTCTGTCTGGAACCAAAGATACTTCGTTATT
TCTAACACCACTGGCTACAATGATCGTGCTGTATTGGAGAGAGAAGTCCAATACACTCTGGAAATGAT
TAAACTAGTACCACATAATGAAAGTGCATGGAACTATTTGAAAGGGATTTTGCAGGATCGTGGTCTTT
CCAAATATCCTAATCTGTTAAATCAATTACTTGATTTACAACCAAGTCATAGTTCCCCCTACCTAATT
GCCTTTCTTGTGGATATCTATGAAGACATGCTAGAAAATCAGTGTGACAATAAGGAAGACATTCTTAA
TAAAGCATTAGAGTTATGTGAAATCCTAGCTAAAGAAAAGGACACTATAAGAAAGGAATATTGGAGAT
ACATTGGAAGATCCCTTCAAAGCAAACACAGCACAGAAAATGACTCACCAACAAATGTACAGCAATAA
CACCATCCAGAAGAACTTGATGGAATGCTTTTATTTTTTATTAAGGGACCCTGCAGGAGTTTCACACG
AGAGTTCCTTCCCTTTTGTGGTGTAAAAGTGCATCACACAGGTATTGCTTTTTACAGACTGATGCTCC
TTGGTGCTGCTGCATCTATCTCAGACTAGCTCTAGTATGTGATCTCTAAGCA
NOV5d,
CG181662-03
Protein Sequence SEQ ID NO: 58 379 aa MW at 44408.2 kD
MAATEGVGEAAQGGEPGQPAQPPPQPHPPPPQQQHKEEMAAEAGEAVASPMDDGFVSLDSPSYVLYRD
RAEWADIDPVPQNDGPNPVVQIIYSDKFRDVYDYFRAVLQRDERSERAFKLTRDAIELNAANYTVWHF
RRVLLKSLQKDLHEEMNYITAIIEEQPKNYQVWHHRRVLVEWLRDPSQELEFIADILNQDAKNYHAWQ
HRQWVIQEFKLWDNELQYVDQLLKEDVRNNSVWNQRYFVISNTTGYNDRAVLEREVQYTLEMIKLVPH
NESAWNYLKGILQDRGLSKYPNLLNQLLDLQPSHSSPYLIAFLVDIYEDMLENQCDNKEDILNKALEL
CEILAKEKDTIRKEYWRYIGRSLQSKHSTENDSPTNVQQ
NOV5e,
CG181662-04 SEQ ID NO: 59 1156 bp
DNA Sequence ORF Start: ATG at 11 ORF Stop: end of sequence
CACCGGATCCATGGCGGCCACCGAGGGGGTCGGGGAGGCTGCGCAAGGGGGCGAGCCCGGGCAGCCGG
CGCAACCCCCGCCCCAGCCGCACCCACCGCCGCCCCAGCAGCAGCACAAGGAAGAGATGGCGGCCGAG
GCTGGGGAAGCCGTGGCGTCCCCCATGGACGACGGGTTTGTGAGCCTGGACTCGCCCTCCTATGTCCT
GTACAGGGACAGAGCAGAATGGGCTGATATAGATCCGGTGCCGCAGAATGATGGCCCCAATCCCGTGG
TCCAGATCATTTATAGTGACAAATTTAGAGATGTTTATGATTACTTCCGAGCTGTCCTGCAGCGTGAT
GAAAGAAGTGAACGAGCTTTTAAGCTAACCCGGGATGCTATTGAGTTAAATGCAGCCAATTATACAGT
GTGGCATTTCCGGAGAGTTCTTTTGAAGTCACTTCAGAAGGATCTACATGAGGAAATGAACTACATCA
CTGCAATAATTGAGGAGCAGCCCAAAAACTATCAAGTTTGGCATCATAGGCGAGTATTAGTGGAATGG
CTAAGAGATCCATCTCAGGAGCTTGAATTTATTGCTGATATTCTTAATCAGGATGCAAAGAATTATCA
TGCCTGGCAGCATCGACAATGGGTTATTCAGGAATTTAAACTTTGGGATAATGAGCTGCAGTATGTGG
ACCAACTTCTGAAAGAGGATGTGAGAAATAACTCTGTCTGGAACCAAAGATACTTCGTTATTTCTAAC
ACCACTGGCTACAATGATCGTGCTGTATTGGAGAGAGAAGTCCAATACACTCTGGAAATGATTAAACT
AGTACCACATAATGAAAGTGCATGGAACTATTTGAAAGGGATTTTGCAGGATCGTGGTCTTTCCAAAT
ATCCTAATCTGTTAAATCAATTACTTGATTTACAACCAAGTCATAGTTCCCCCTACCTAATTGCCTTT
CTTGTGGATATCTATGAAGACATGCTAGAAAATCAGTGTGACAATAAGGAAGACATTCTTAATAAAGC
ATTAGAGTTATGTGAAATCCTAGCTAAAGAAAAGGACACTATAAGAAAGGAATATTGGAGATACATTG
GAAGATCCCTTCAAAGCAAACACAGCACAGAAAATGACTCACCAACAAATGTACAGCAA
NOV5e,
CG181662-04
Protein Sequence SEQ ID NO: 60 379 aa MW at 44408.2 kD
MAATEGVGEAAQGGEPGQPAQPPPQPHPPPPQQQHKEEMAAEAGEAVASPMDDGFVSLDSPSYVLYRD
RAEWADIDPVPQNDGPNPVVQIIYSDKFRDVYDYFRAVLQRDERSERAFKLTRDAIELNAANYTVWHF
RRVLLKSLQKDLHEEMNYITAIIEEQPKNYQVWHHRRVLVEWLRDPSQELEFIADILNQDAKNYHAWQ
HRQWVIQEFKLWDNELQYVDQLLKEDVRNNSVWNQRYFVISNTTGYNDRAVLEREVQYTLEMIKLVPN
NESAWNYLKGILQDRGLSKYPNLLNQLLDLQPSHSSPYLIAFLVDIYEDMLENQCDNKEDILNKALEL
CEILAKEKDTIRKEYWRYIGRSLQSKHSTENDSPTNVQQ
NOV5f,
13382357 SNP 1492 bp, SNP
CG181662-01 SEQ ID NO: 61 at position 310 C/T
DNA Sequence ORF Start: ATG at 4 ORF Stop: TAA at 940
GAGATGGCGGCCACCGAGGGGGTCGGGGAGGCTGCGCAAGGGGGCGAGCCCGGGCAGCCGGCGCAACCCC
CGCCCCAGCCGCACCCACCGCCGCCCCAGCAGCAGCACAAGGAAGAGATGGCGGCCGAGGCTGGGGAAGC
CGTGGCGTCCCCCATGGACGACGGGTTTGTGAGCCTGGACTCGCCCTCCTATGTCCTATACAGGCATTTC
CGGAGAGTTCTTTTGAAGTCACTTCAGAAGGATCTACATGAGGAAATGAACTACATCACTGCAATAATTG
AGGAGCAGCCCAAAAACTATCAAGTTTGGTATCATAGGCGAGTATTAGTGGAATGGCTAAGAGATCCATC
TCAGGAGCTTGAATTTATTGCTGATATTCTTAATCAGGATGCAAAGAATTATCATGCCTGGCAGCATCGA
CAATGGGTTATTCAGGAATTTAAACTTTGGGATAATGAGCTGCAGTATGTGGACCAACTTCTGAAAGAGG
ATGTGAGAAATAACTCTGTCTGGAACCAAAGATACTTCGTTATTTCTAACACCACTGGCTACAATGATCG
TGCTGTATTGGAGAGAGAAGTCCAATACACTCTGGAAATGATTAAACTAGTACCACATAATGAAAGTGCA
TGGAACTATTTGAAAGGGATTTTGCAGGATCGTGGTCTTTCCAAATATCCTAATCTGTTAAATCAATTAC
TTGATTTACAACCAAGTCATAGTTCCCCCTACCTAATTGCCTTTCTTGTGGATATCTATGAAGACATGCT
AGAAAATCAGTGTGACAATAAGGAAGACATTCTTAATAAAGCATTAGAGTTATGTGAAATCCTAGCTAAA
GAAAAGGACACTATAAGAAAGGAATATTGGAGATACATTGGAAGATCCCTTCAAAGCAAACACAGCACAG
AAAATGACTCACCAACAAATGTACAGCAATAACACCATCCAGAAGAACTTGATGGAATGCTTTTATTTTT
TATTAAGGGACCCTGCAGGAGTTTCACACGAGAGTGGTCCTTCCCTTTGCCTGTGGTGTAAAAGTGCATC
ACACAGGTATTGCTTTTTAACAAGAACTGATGCTCCTTGGGTGCTGCTGCTACTCAGACTAGCTCTAAGT
AATGTGATTCTTCTAAAGCAAAGTCATTGGATGGGAGGAGGAAGAAAAAGTCCCATAAAGGAACTTTTGT
AGTCTTATCAACATATAATCTAATCCCTTAGCATCAGCTCCTCCCTCAGTGGTACATGCGTCAAGATTTG
TAGCAGTAATAACTGCAGGTCACTTGTATGTAATGGATGTGAGGTAGCCGAAGTTTGGTTCAGTAAGCAG
GGAATACAGTCGTTCCATCAGAGCTGGTCTGCACACTCACATTATCTTGCTATCACTGTAACCAACTAAT
GCCAAAAGAACGGTTTTGTAATAAAATTATAGCTGTATCTAAAAAAAAAAAAAAAAAAAAAAAAAAAAAA
AAAAAAAAAAAAAAAAAACAAA
NOV5f,
13382357 SNP
CG181662-01 SNP: His to Tyr
Protein Sequence SEQ ID NO: 62 312 aa at position 103
MAATEGVGEAAQGGEPGQPAQPPPQPHPPPPQQQHKEEMAAEAGEAVASPMDDGFVSLDSPSYVLYRHFR
RVLLKSLQKDLHEEMNYITAIIEEQPKNYQVWYHRRVLVEWLRDPSQELEFIADILNQDAKNYHAWQHRQ
WVIQEFKLWDNELQYVDQLLKEDVRNNSVWNQRYFVISNTTGYNDRAVLEREVQYTLEMIKLVPHNESAW
NYLKGILQDRGLSKYPNLLNQLLDLQPSHSSPYLIAFLVDIYEDMLENQCDNKEDILNKALELCEILAKE
KDTIRKEYWRYIGRSLQSKHSTENDSPTNVQQ
NOV5g,
13377970 SNP 1492 bp, SNP
CG181662-01 SEQ ID NO: 63 at position 457 G/C

| | |
|---|---|
| DNA Sequence ORF Start ATG at 4 | ORF Stop: TAA at 940 |
| GAGATGGCGGCCACCGAGGGGGTCGGGGAGGCTGCGCAAGGGGGCGAGCCCGGGCAGCCGGCGCAACCCC | |
| CGCCCCAGCCGCACCCACCGCCGCCCCAGCAGCAGCACAAGGAAGAGATGGCGGCCGAGGCTGGGGAAGC | |
| CGTGGCGTCCCCCATGGACGACGGGTTTGTGAGCCTGGACTCGCCCTCCTATGTCCTATACAGGCATTTC | |
| CGGAGAGTTCTTTTGAAGTCACTTCAGAAGGATCTACATGAGGAAATGAACTACATCACTGCAATAATTG | |
| AGGAGCAGCCCAAAAACTATCAAGTTTGGCATCATAGGCGAGTATTAGTGGAATGGCTAAGAGATCCATC | |
| TCAGGAGCTTGAATTTATTGCTGATATTCTTAATCAGGATGCAAAGAATTATCATGCCTGGCAGCATCGA | |
| CAATGGGTTATTCAGGAATTTAAACTTTGGGATAATCAGCTGCAGTATGTGGACCAACTTCTGAAAGAGG | |
| ATGTGAGAAATAACTCTGTCTGGAACCAAAGATACTTCGTTATTTCTAACACCACTGGCTACAATGATCG | |
| TGCTGTATTGGAGAGAGAAGTCCAATACACTCTGGAAATGATTAAACTAGTACCACATAATGAAAGTGCA | |
| TGGAACTATTTGAAAGGGATTTTGCAGGATCGTGGTCTTTCCAAATATCCTAATCTGTTAAATCAATTAC | |
| TTGATTTACAACCAAGTCATAGTTCCCCCTACCTAATTGCCTTTCTTGTGGATATCTATGAAGACATGCT | |
| AGAAAATCAGTGTGACAATAAGGAAGACATTCTTAATAAAGCATTAGAGTTATGTGAAATCCTAGCTAAA | |
| GAAAAGGACACTATAAGAAAGGAATATTGGAGATACATTGGAAGATCCCTTCAAAGCAAACACAGCACAG | |
| AAAATGACTCACCAACAAATGTACAGCAATAACACCATCCAGAAGAACTTGATGCAATGCTTTTATTTTT | |
| TATTAAGGGACCCTGCAGGAGTTTCACACGAGAGTGGTCCTTCCCTTTGCCTGTGGTGTAAAAGTGCATC | |
| ACACAGGTATTGCTTTTTAACAAGAACTGATGCTCCTTGGGTGCTGCTGCTACTCAGACTAGCTCTAAGT | |
| AATGTGATTCTTCTAAAGCAAAGTCATTGGATGGGAGGAGGAAGAAAAAGTCCCATAAAGGAACTTTTGT | |
| AGTCTTATCAACATATAATCTAATCCCTTAGCATCAGCTCCTCCCTCAGTGGTACATGCGTCAAGATTTG | |
| TAGCAGTAATAACTGCAGGTCACTTGTATGTAATGGATGTGAGGTAGCCGAAGTTTGGTTCAGTAAGCAG | |
| GGAATACAGTCGTTCCATCAGAGCTGGTCTGCACACTCACATTATCTTGCTATCACTGTAACCAACTAAT | |
| GCCAAAAGAACGGTTTTGTAATAAAATTATAGCTGTATCTAAAAAAAAAAAAAAAAAAAAAAAAAAAAAA | |
| AAAAAAAAAAAAAAAAAACAAA | |
| NOV5g, | |
| 13377970 SNP | |
| CG181662-01 SNP: Glu to Gln | |
| Protein Sequence SEQ ID NO: 64 312 aa at position 152 | |
| MAATEGVGEAAQGGEPGQPAQPPPQPHPPPPQQQHKEEMAAEAGEAVASPMDDGFVSLDSPSYVLYRHFR | |
| RVLLKSLQKDLHEEMNYITAIIEEQPKNYQVWHHRRVLVEWLRDPSQELEFIADILNQDAKNYHAWQHRQ | |
| WVIQEFKLWDNQLQYVDQLLKEDVRNNSVWNQRYFVISNTTGYNDRAVLEREVQYTLEMIKLVPHNESAW | |
| NYLKGILQDRGLSKYPNLLNQLLDLQPSHSSPYLIAFLVDIYEDMLENQCDNKEDILNKALELCEILAKE | |
| KDTIRKEYWRYIGRSLQSKHSTENDSPTNVQQ | |
| NOV5h, | |
| 13378241 SNP 1492 bp, SNP | |
| CG181662-01 SEQ ID NO: 65 at position 729 C/A | |
| DNA Sequence ORF Start: ATG at 4 ORF Stop: TAA at 940 | |
| GAGATGGCGGCCACCGAGGGGGTCGGGGAGGCTGCGCAAGGGGGCGAGCCCGGGCAGCCGGCGCAACCCC | |
| CGCCCCAGCCGCACCCACCGCCGCCCCAGCAGCAGCACAAGGAAGAGATGGCGGCCGAGGCTGGGGAAGC | |
| CGTGGCGTCCCCCATGGACGACGGGTTTGTGAGCCTGGACTCGCCCTCCTATGTCCTATACAGGCATTTC | |
| CGGAGAGTTCTTTTGAAGTCACTTCAGAAGGATCTACATGAGGAAATGAACTACATCACTGCAATAATTG | |
| AGGAGCAGCCCAAAAACTATCAAGTTTGGCATCATAGGCGAGTATTAGTGGAATGGCTAAGAGATCCATC | |
| TCAGGAGCTTGAATTTATTGCTGATATTCTTAATCAGGATGCAAAGAATTATCATGCCTGGCAGCATCGA | |
| CAATGGGTTATTCAGGAATTTAAACTTTGGGATAATGAGCTGCAGTATGTGGACCAACTTCTGAAAGAGG | |
| ATGTGAGAAATAACTCTGTCTGGAACCAAAGATACTTCGTTATTTCTAACACCACTGGCTACAATGATCG | |
| TGCTGTATTGGAGAGAGAAGTCCAATACACTCTGGAAATGATTAAACTAGTACCACATAATGAAAGTGCA | |
| TGGAACTATTTGAAAGGGATTTTGCAGGATCGTGGTCTTTCCAAATATCCTAATCTGTTAAATCAATTAC | |
| TTGATTTACAACCAAGTCATAGTTCCCCATACCTAATTGCCTTTCTTGTGGATATCTATGAAGACATGCT | |
| AGAAAATCAGTGTGACAATAAGGAAGACATTCTTAATAAAGCATTAGAGTTATGTGAAATCCTAGCTAAA | |
| GAAAAGGACACTATAAGAAAGGAATATTGGAGATACATTGGAAGATCCCTTCAAAGCAAACACAGCACAG | |
| AAAATGACTCACCAACAAATGTACAGCAATAACACCATCCAGAAGAACTTGATGGAATGCTTTTATTTTT | |
| TATTAAGGGACCCTGCAGGAGTTTCACACGAGAGTGGTCCTTCCCTTTGCCTGTGGTGTAAAAGTGCATC | |
| ACACAGGTATTGCTTTTTAACAAGAACTGATGCTCCTTGGGTGCTGCTGCTACTCAGACTAGCTCTAAGT | |
| AATGTGATTCTTCTAAAGCAAAGTCATTGGATGGGAGGAGGAAGAAAAAGTCCCATAAAGGAACTTTTGT | |
| AGTCTTATCAACATATAATCTAATCCCTTAGCATCAGCTCCTCCCTCAGTGGTACATGCGTCAAGATTTG | |
| TAGCAGTAATAACTGCAGGTCACTTGTATGTAATGGATGTGAGGTAGCCGAAGTTTGGTTCAGTAAGCAG | |
| GGAATACAGTCGTTCCATCAGAGCTGGTCTGCACACTCACATTATCTTGCTATCACTGTAACCAACTAAT | |
| GCCAAAAGAACGGTTTTGTAATAAAATTATAGCTGTATCTAAAAAAAAAAAAAAAAAAAAAAAAAAAAAA | |
| AAAAAAAAAAAAAAAAAACAAA | |
| NOV5h, | |
| 13378241 SNP | |
| CG181662-01 SNP: no change in the | |
| Protein Sequence SEQ ID NO: 66 312 aa protein sequence | |
| MAATEGVGEAAQGGEPGQPAQPPPQPHPPPPQQQHKEEMAAEAGEAVASPMDDGFVSLDSPSYVLYRHFR | |
| RVLLKSLQKDLHEEMNYITAIIEEQPKNYQVWHHRRVLVEWLRDPSQELEFIADILNQDAKNYHAWQHRQ | |
| WVIQEFKLWDNELQYVDQLLKEDVRNNSVWNQRYFVISNTTGYNDRAVLEREVQYTLEMIKLVPHNESAW | |
| NYLKGILQDRGLSKYPNLLNQLLDLQPSHSSPYLIAFLVDIYEDMLENQCDNKEDILNKALELCEILAKE | |
| KDTIRKEYWRYIGRSLQSKHSTENDSPTNVQQ | |
| NOV5i, | |
| 13377901 SNP 1492 bp, SNP | |
| CG181662-01 SEQ ID NO: 67 at position | 1330 G/T |

DNA Sequence ORF Start: ATG at 4 ORF Stop: TAA at 940
GAGATGGCGGCCACCGAGGGGGTCGGGGAGGCTGCGCAAGGGGGCGAGCCCGGGCAGCCGGCGCAACCCC
CGCCCCAGCCGCACCCACCGCCGCCCCAGCAGCAGCACAAGGAAGAGATGGCGGCCGAGGCTGGGGAAGC
CGTGGCGTCCCCCATGGACGACGGGTTTGTGAGCCTGGACTCGCCCTCCTATGTCCTATACAGGCATTTC
CGGAGAGTTCTTTTGAAGTCACTTCAGAAGGATCTACATGAGGAAATGAACTACATCACTGCAATAATTG
AGGAGCAGCCCAAAAACTATCAAGTTTGGCATCATAGGCGAGTATTAGTGGAATGGCTAAGAGATCCATC
TCAGGAGCTTGAATTTATTGCTGATATTCTTAATCAGGATGCAAAGAATTATCATGCCTGGCAGCATCGA
CAATGGGTTATTCAGGAATTTAAACTTTGGGATAATGAGCTGCAGTATGTGGACCAACTTCTGAAAGAGG
ATGTGAGAAATAACTCTGTCTGGAACCAAAGATACTTCGTTATTTCTAACACCACTGGCTACAATGATCG
TGCTGTATTGGAGAGAGAAGTCCAATACACTCTGGAAATGATTAAACTAGTACCACATAATGAAAGTGCA
TGGAACTATTTGAAAGGGATTTTGCAGGATCGTGGTCTTTCCAAATATCCTAATCTGTTAAATCAATTAC
TTGATTTACAACCAAGTCATAGTTCCCCCTACCTAATTGCCTTTCTTGTGGATATCTATGAAGACATGCT
AGAAAATCAGTGTGACAATAAGGAAGACATTCTTAATAAAGCATTAGAGTTATGTGAAATCCTAGCTAAA
GAAAAGGACACTATAAGAAAGGAATATTGGAGATACATTGGAAGATCCCTTCAAAGCAAACACAGCACAG
AAAATGACTCACCAACAAATGTACAGCAATAACACCATCCAGAAGAACTTGATGGAATGCTTTTATTTTT
TATTAAGGGACCCTGCAGGAGTTTCACACGAGAGTGGTCCTTCCCTTTGCCTGTGGTGTAAAAGTGCATC
ACACAGGTATTGCTTTTTAACAAGAACTGATGCTCCTTGGGTGCTGCTGCTACTCAGACTAGCTCTAAGT
AATGTGATTCTTCTAAAGCAAAGTCATTGGATGGGAGGAGGAAGAAAAAGTCCCATAAAGGAACTTTTGT
AGTCTTATCAACATATAATCTAATCCCTTAGCATCAGCTCCTCCCTCAGTGGTACATGCGTCAAGATTTG
TAGCAGTAATAACTGCAGGTCACTTGTATGTAATGGATGTGAGGTAGCCGAAGTTTGGTTCAGTAAGCAT
GGAATACAGTCGTTCCATCAGAGCTGGTCTGCACACTCACATTATCTTGCTATCACTGTAACCAACTAAT
GCCAAAAGAACGGTTTTGTAATAAAATTATAGCTGTATCTAAAAAAAAAAAAAAAAAAAAAAAAAAAAAA
AAAAAAAAAAAAAAAAAACAAA
NOV5i,
13377901 SNP
CG181662-O1
Protein Sequence SEQ ID NO: 68 312 aa SNP: Not in coding region
MAATEGVGEAAQGGEPGQPAQPPPQPHPPPPQQQHKEEMAAEAGEAVASPMDDGFVSLDSPSYVLYRHF
RVLLKSLQKDLHEEMNYITAIIEEQPKNYQVWHHRRVLVEWLRDPSQELEFIADILNQDAKNYHAWQHQ
RQWVIQEFKLWDNELQYVDQLLKEDVRNNSVWNQRYFVISNTTGYNDRAVLEREVQYTLEMIKLVPHNE
SAWNYLKGILQDRGLSKYPNLLNQLLDLQPSHSSPYLIAFLVDIYEDMLENQCDNKEDILNKALELCEI
LAKEKDTIRKEYWRYIGRSLQSKHSTENDSPTNVQQ
NOV5j,
13377900 SNP 1492 bp, SNP
CG181662-O1 SEQ ID NO: 69 at position 1385 A/C
DNA Sequence ORF Start: ATG at 4 ORF Stop: TAA at 940
GAGATGGCGGCCACCGAGGGGGTCGGGGAGGCTGCGCAAGGGGGCGAGCCCGGGCAGCCGGCGCAACCC
CCGCCCCAGCCGCACCCACCGCCGCCCCAGCAGCAGCACAAGGAAGAGATGGCGGCCGAGGCTGGGGAA
GCCGTGGCGTCCCCCATGGACGACGGGTTTGTGAGCCTGGACTCGCCCTCCTATGTCCTATACAGGCAT
TTCCGGAGAGTTCTTTTGAAGTCACTTCAGAAGGATCTACATGAGGAAATGAACTACATCACTGCAATA
ATTGAGGAGCAGCCCAAAAACTATCAAGTTTGGCATCATAGGCGAGTATTAGTGGAATGGCTAAGAGAT
CCATCTCAGGAGCTTGAATTTATTGCTGATATTCTTAATCAGGATGCAAAGAATTATCATGCCTGGCAG
CATCGACAATGGGTTATTCAGGAATTTAAACTTTGGGATAATGAGCTGCAGTATGTGGACCAACTTCTG
AAAGAGGATGTGAGAAATAACTCTGTCTGGAACCAAAGATACTTCGTTATTTCTAACACCACTGGCTAC
AATGATCGTGCTGTATTGGAGAGAGAAGTCCAATACACTCTGGAAATGATTAAACTAGTACCACATAAT
GAAAGTGCATGGAACTATTTGAAAGGGATTTTGCAGGATCGTGGTCTTTCCAAATATCCTAATCTGTTA
AATCAATTACTTGATTTACAACCAAGTCATAGTTCCCCCTACCTAATTGCCTTTCTTGTGGATATCTAT
GAAGACATGCTAGAAAATCAGTGTGACAATAAGGAAGACATTCTTAATAAAGCATTAGAGTTATGTGAA
ATCCTAGCTAAAGAAAAGGACACTATAAGAAAGGAATATTGGAGATACATTGGAAGATCCCTTCAAAGC
AAACACAGCACAGAAAATGACTCACCAACAAATGTACAGCAATAACACCATCCAGAAGAACTTGATGGA
ATGCTTTTATTTTTTATTAAGGGACCCTGCAGGAGTTTCACACGAGAGTGGTCCTTCCCTTTGCCTGTG
GTGTAAAAGTGCATCACACAGGTATTGCTTTTTAACAAGAACTGATGCTCCTTGGGTGCTGCTGCTACT
CAGACTAGCTCTAAGTAATGTGATTCTTCTAAAGCAAAGTCATTGGATGGGAGGAGGAAGAAAAAGTCC
CATAAAGGAACTTTTGTAGTCTTATCAACATATAATCTAATCCCTTAGCATCAGCTCCTCCCTCAGTGG
TACATGCGTCAAGATTTGTAGCAGTAATAACTGCAGGTCACTTGTATGTAATGGATGTGAGGTAGCCGA
AGTTTGGTTCAGTAAGCAGGGAATACAGTCGTTCCATCAGAGCTGGTCTGCACACTCACATTATCTTGC
TATCCCTGTAACCAACTAATGCCAAAAGAACGGTTTTGTAATAAAATTATAGCTGTATCTAAAAAAAAA
AAAAAAAAAAAAAAAAAAAAAAAAAAAAAAAAAAAAAAACAAA
NOV5j,
13377900 SNP
CG181662-01
Protein Sequence SEQ ID NO: 70 312 aa SNP: Not in coding region
MAATEGVGEAAQGGEPGQPAQPPPQPHPPPPQQQHKEEMAAEAGEAVASPMDDGFVSLDSPSYVLYRHFR
RVLLKSLQKDLHEEMNYITAIIEEQPKNYQVWHHRRVLVEWLRDPSQELEFIADILNQDAKNYHAWQHRQ
WVIQEFKLWDNELQYVDQLLKEDVRNNSVWNQRYFVISNTTGYNDRAVLEREVQYTLEMIKLVPHNESAW
NYLKGILQDRGLSKYPNLLNQLLDLQPSHSSPYLIAFLVDIYEDMLENQCDNKEDILNKALELCEILAKE
KDTIRKEYWRYIGRSLQSKHSTENDSPTNVQQ

A ClustalW comparison of the above protein sequences yields the following sequence alignment shown in Table 5B.

TABLE 5B
Comparison of the NOV5 protein sequences.

| | |
|---|---|
| NOV5a | -----MAATEGVGEAAQGGEPGQPAQPPPQPHPPPPQQQHKEEMAAEAGEAVASPMDDGF |
| NOV5b | -----MAATEGVGEAAQGGEPGQPAQPPPQPHPPPPQQQHKEEMAAEACEAVASPMDDGF |
| NOV5c | GRVDEMAATEGVGEAAQGGEPGQPAQPPPQPHPPPPQQQHKEEMAAEAGEAVASPMDDCF |
| NOV5d | -----MAATEGVGEAAQGGEPGQPAQPPPQPHPPPPQQQHKEEMAAEAGEAVASPMDDGF |
| NOV5e | -----MAATEGVGEAAQGGEPGQPAQPPPQPHPPPPQQQHKEEMAAEAGEAVASPMDDGF |
| NOV5a | VSLDSPSYVLYR------------------------------------------------ |
| NOV5b | VSLDSPSYVLYR------------------------------------------------ |
| NOV5c | VSLDSPSYVLYR------------------------------------------------ |
| NOV5d | VSLDSPSYVLYRDRAEWADIDPVPQNDGPNPVVQIIYSDKFRDVYDYFRAVLQRDERSER |
| NOV5e | VSLDSPSYVLYRDRAEWADIDPVPQNDGPNPVVQIIYSDKFRDVYDYFRAVLQRDERSER |
| NOV5a | -------------------HFRRVLLKSLQKDLHEEMNYITAIIEEQPKNYQVWHHRRVL |
| NOV5b | -------------------HFRRVLLKSLQKDLHEEMNYITAIIEEQPKNYQVWHHRRVL |
| NOV5c | -------------------HFRRVLLKSLQKDLHEEMNYITAIIEEQPKNYQVWHHRRVL |
| NOV5d | AFKLTRDAIELNAANYTVWHFRRVLLKSLQKDLHEEMNYITAIIEEQPKNYQVWHHRRVL |
| NOV5e | AFKLTRDAIELNAANYTVWHFRRVLLKSLQKDLHEEMNYITAIIEEQPKNYQVWHHRRVL |
| NOV5a | VEWLRDPSQELEFIADILNQDAKNYHAWQHRQWVIQEFKLWDNELQYVDQLLKEDVRNNS |
| NOV5b | VEWLRDPSQELEFIADILNQDAKNYHAWQHRQWVIQEFKLWDNELQYVDQLLKEDVRNNS |
| NOV5c | VEWLRDPSQELEFIADILNQDAKNYHAWQHRQWVIQEFKLWDNELQYVDQLLKEDVRNNS |
| NOV5d | VEWLRDPSQELEFIADILNQDAKNYEAWQHRQWVIQEFKLWDNELQYVDQLLKEDVRNNS |
| NOV5e | VEWLRDPSQELEFIADILNQDAKNYEAWQHRQWVIQEFKLWDNELQYVDQLLKEDVRNNS |
| NOV5a | VWNQRYFVISNTTGYNDRAVLEREVQYTLEMTKLVPHNESAWNYLKGILQDRGLSKYPNL |
| NOV5b | VWNQRYFVISNTTGYNDRAVLEREVQYTLEMTKLVPHNESAWNYLKGILQDRGLSKYPNL |
| NOV5c | VWNQRYFVISNTTCYNDRAVLEREVQYTLEMIKLVPHNESAWNYLKGILQDRGLSKYPNL |
| NOV5d | VWNQRYFVISNTTGYNDRAVLEREVQYTLEMIKLVPHNESAWNYLKGILQDRGLSKYFNL |
| NOV5e | VWNQRYFVISNTTGYNDRAVLEREVQYTLEMIKLVPHNESAWNYLKGILQDRGLSKYPNL |
| NOV5a | LNQLLDLQPSHSSPYLIAFLVDIYEDMLENQCDNKEDILNKALELCEILAKEKDTIRKEY |
| NOV5b | LNQLLDLQPSHSSPYLIAFLVDIYEDMLENQCDNKEDILNKALELCEILAKEKDTIRKEY |
| NOV5c | LNQLLDLQPSHSSPYLIAFLVDIYEDMLENQCDNKEDILNKALELCEILAKEKDTIRKEY |
| NOV5d | LNQLLDLQPSHSSPYLIAFLVDIYEDMLENQCDNKEDILNKALELCETLAKEKDTTRKEY |
| NOV5e | LNQLLDLQPSHSSPYLIAFLVDIYEDMLENQCDNKEDILNKALELCEILAKEKDTIRKEY |
| NOV5a | WRYIGRSLQSKHSTENDSPTNVQQ |
| NOV5b | WRYIGRSLQSKHSTENDSPTNVQQ |
| NOV5c | WRYIGRSLQSKHSTENDSPTNVQQ |
| NOV5d | WRYIGRSLQSKHSTENDSPTNVQQ |
| NOV5e | WRYIGRSLQSKHSTENDSPTNVQQ |
| NOV5a | (SEQ ID NO: 52) |
| NOV5b | (SEQ ID NO: 54) |
| NOV5c | (SEQ ID NO: 56) |
| NOV5d | (SEQ ID NO: 58) |
| NOV5e | (SEQ ID NO: 60) |

Further analysis of the NOV5a protein yielded the following properties shown in Table 5C.

TABLE 5C
Protein Sequence Properties NOV5a

| SignalP | No Known Signal Sequence Predicted | | | |
|---|---|---|---|---|
| analysis: | | | | |
| PSORT II | PSG: a new signal peptide prediction method | | | |
| analysis: | N-region: length 9; pos. chg 0; neg. chg 2 | | | |
| | H-region: length 5; peak value 0.00 | | | |
| | PSG score: −4.40 | | | |
| | GvH: von Heijne's method for signal seq. recognition | | | |
| | GvH score (threshold: −2.1): −11.19 | | | |
| | possible cleavage site: between 13 and 14 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 0 | | | |
| | number of TMS(s) . . . fixed | | | |
| | PERIPHERAL Likelihood = 6.42 (at 240) | | | |
| | ALOM score: 6.42 (number of TMSs: 0) | | | |
| | MITDISC: discrimination of mitochondrial targeting seq | | | |
| | R content: | 0 | Hyd Moment(75): | 6.27 |
| | Hyd Moment(95): | 4.56 | G content: | 2 |
| | D/E content: | 2 | S/T content: | 1 |
| | Score: −7.86 | | | |

| Gavel: prediction of cleavage sites for mitochondrial preseq | |
|---|---|
| cleavage site motif not found | |
| NUCDISC: discrimination of nuclear localization signals | |
| pat4: none | |
| pat7: none | |
| bipartite: none | |
| content of basic residues: 9.9% | |
| NLS Score: −0.47 | |
| KDEL: ER retention motif in the C-terminus: none | |
| ER Membrane Retention Signals: none | |
| SKL: peroxisomal targeting signal in the C-terminus: none | |
| PTS2: 2nd peroxisomal targeting signal: none | |
| VAC: possible vacuolar targeting motif: none | |
| RNA-binding motif: none | |
| Actinin-type actin-binding motif: | |
| type 1: none | |
| type 2: none | |
| NMYR: N-myristoylation pattern: none | |
| Prenylation motif: none | |
| memYQRL: transport motif from cell surface to Golgi: none | |
| Tyrosines in the tail: none | |
| Dileucine motif in the tail: none | |
| checking 63 PROSITE DNA binding motifs: none | |
| checking 71 PROSITE ribosomal protein motifs: none | |
| checking 33 PROSITE prokaryotic DNA binding motifs: none | |
| NNCN: Reinhardt's method for Cytoplasmic/Nuclear discrimination | |
| Prediction: nuclear | |
| Reliability: 55.5 | |
| COIL: Lupas's algorithm to detect coiled-coil regions | |
| 249 D | 0.58 |
| 250 I | 0.58 |
| 251 Y | 0.82 |
| 252 E | 0.82 |
| 253 D | 0.93 |
| 254 M | 0.93 |
| 255 L | 0.97 |
| 256 E | 0.97 |
| 257 N | 0.97 |
| 258 Q | 0.97 |
| 259 C | 0.97 |
| 260 D | 0.97 |
| 261 N | 0.97 |
| 262 K | 0.97 |
| 263 E | 0.97 |
| 264 D | 0.97 |
| 265 I | 0.97 |
| 266 L | 0.97 |
| 267 N | 0.97 |
| 268 K | 0.97 |
| 269 A | 0.97 |
| 270 L | 0.97 |
| 271 E | 0.97 |
| 272 L | 0.97 |
| 273 C | 0.97 |
| 274 E | 0.97 |
| 275 I | 0.97 |
| 276 L | 0.97 |
| 277 A | 0.97 |
| 278 K | 0.97 |
| 279 E | 0.97 |
| 280 K | 0.97 |
| 281 D | 0.97 |
| 282 T | 0.97 |
| 283 I | 0.86 |
| 284 R | 0.70 |
| 285 K | 0.70 |
| 286 E | 0.70 |
| 287 Y | 0.70 |

```
total: 39 residues
Final Results (k = 9/23):
78.3%: nuclear
  8.7%: mitochondrial
  8.7%: cytoplasmic
  4.3%: peroxisomal
>> prediction for CG181662-01 is nuc (k = 23)
```

A search of the NOV5a protein against the Geneseq database, a proprietary database that contains sequences published in patents and patent publication, yielded several homologous proteins shown in Table 5D.

TABLE 5D
Geneseq Results for NOV5a

| | | NOV5a | Identities/ | |
|---|---|---|---|---|
| | | Residues/ | Similarities for | |
| Geneseq | Protein/Organism/Length [Patent | Match | the Matched | Expect |
| Identifier | #, Date] | Residues | Region | Value |
| AAB58384 | Lung cancer associated polypeptide | 1 . . . 312 | 278/380 (73%) | e−152 |
| | sequence SEQ ID 722 - Homo | 16 . . . 394 | 289/380 (75%) | |
| | sapiens, 394 aa. [WO200055180-A2, | | | |
| | 21 SEP. 2000] | | | |
| ABB08436 | Protein sequence 2 relative to the | 1 . . . 312 | 278/380 (73%) | e−152 |
| | farnesyltransferase of the invention - | 1 . . . 379 | 289/380 (75%) | |
| | Unidentified, 379 aa. [KR98075770- | | | |
| | A, 16 NOV. 1998] | | | |
| AAU77150 | Human geranylgeranyltransferase | 1 . . . 312 | 278/380 (73%) | e−152 |
| | type I related protein #2 - | 1 . . . 379 | 289/380 (75%) | |
| | Unidentified, 380 aa. [KR98075771- | | | |
| | A, 16 NOV. 1998] | | | |
| AAW04431 | Human farnesyl transferase enzyme | 1 . . . 312 | 278/380 (73%) | e−152 |
| | alpha subunit - Homo sapiens, 379 | 1 . . . 379 | 289/380 (75%) | |
| | aa. [WO9634113-A2, 31 OCT. 1996] | | | |
| AAR77841 | Human farnesyl protein transferase | 1 . . . 312 | 278/380 (73%) | e−152 |
| | alpha subunit - Homo sapiens, 379 | 1 . . . 379 | 289/380 (75%) | |
| | aa. [US5420245-A, 30 MAY 1995] | | | |

In a BLAST search of public sequence databases, the NOV5a protein was found to have homology to the proteins shown in the BLASTP data in Table 5E.

TABLE 5E
Public BLASTP Results for NOV5a

| | | NOV5a | Identities/ | |
|---|---|---|---|---|
| Protein | | Residues/ | Similarities for | |
| Accession | | Match | the Matched | Expect |
| Number | Protein/Organism/Length | Residues | Portion | Value |
| P49354 | Protein farnesyltransferase alpha | 1 . . . 312 | 278/380 (73%) | e−152 |
| | subunit (EC 2.5.1.-) (CAAX | 1 . . . 379 | 289/380 (75%) | |
| | farnesyltransferase alpha subunit) (RAS | | | |
| | proteins prenyltransferase alpha) | | | |
| | (FTase-alpha) - Homo sapiens | | | |
| | (Human), 379 aa. | | | |
| P29702 | Protein farnesyltransferase alpha | 56 . . . 312 | 242/257 (94%) | e−143 |
| | subunit (EC 2.5.1.-) (CAAX | 85 . . . 340 | 251/257 (97%) | |
| | farnesyltransferase alpha subunit) (RAS | | | |
| | proteins prenyltransferase alpha) | | | |
| | (FTase-alpha) - Bos taurus (Bovine), | | | |
| | 340 aa (fragment). | | | |
| Q04631 | Protein farnesyltransferase alpha | 1 . . . 310 | 258/378 (68%) | e−139 |
| | subunit (EC 2.5.1.-) (CAAX | 1 . . . 377 | 277/378 (73%) | |
| | farnesyltransferase alpha subunit) (RAS | | | |
| | proteins prenyltransferase alpha) | | | |
| | (FTase-alpha) - Rattus norvegicus | | | |
| | (Rat), 377 aa. | | | |
| Q61239 | Protein farnesyltransferase alpha | 1 . . . 310 | 256/378 (67%) | e−139 |
| | subunit (EC 2.5.1.-) (CAAX | 1 . . . 377 | 277/378 (72%) | |
| | farnesyltransferase alpha subunit) (RAS | | | |
| | proteins prenyltransferase alpha) | | | |
| | (FTase-alpha) - Mus musculus (Mouse), | | | |
| | 377 aa. | | | |
| Q921F7 | Similar to farnesyltransferase, CAAX | 1 . . . 310 | 255/378 (67%) | e−138 |
| | box, alpha - Mus musculus (Mouse), | 1 . . . 377 | 277/378 (72%) | |
| | 377 aa. | | | |

PFam analysis predicts that the NOV5a protein contains the domains shown in the Table 5F.

TABLE 5F
Domain Analysis of NOV5a

| | | Identities/ | |
|---|---|---|---|
| | NOV5a | Similarities for | |
| Pfam | Match | the Matched | Expect |
| Domain | Region | Region | Value |
| PPTA | 83 . . . 113 | 12/31 (39%) | 3.3e−11 |
| | | 28/31 (90%) | |
| PPTA | 117 . . . 147 | 12/31 (39%) | 4e−12 |
| | | 29/31 (94%) | |
| PPTA | 151 . . . 181 | 9/31 (29%) | 2.8e−09 |
| | | 29/31 (94%) | |
| PPTA | 191 . . . 221 | 15/31 (48%) | 1.7e−09 |
| | | 28/31 (90%) | |

### Example 6

The NOV6 clone was analyzed, and the nucleotide and encoded polypeptide sequences are shown in Table 6A.

TABLE 6A
NOV6 Sequence Analysis

NOV6a, CG182223-01 SEQ ID NO: 71 4683 bp
DNA Sequence ORF Start: ATG at 7 ORF Stop: TAA at 4588
GTCAAAATGAGTCTGCTGATGTTTACACAACTACTGCTCTGTGGATTTTTATATGTTCGGGTTGATGG
ATCGCGTCTTCGCCAGGAGGACTTTCCCCCGCGGATTGTGGAGCATCCTTCCGATGTCATCGTCTCTA
AGGGCGACCCCACGACTCTGAACTGCAAGGCGGAGGGCCGGCCAACGCCCACCATTGAGTGGTACAAA
GATGGGGAGCGAGTGGAGACTGACAAGCACCATCCCCGGTCCCACAGGATGCTTCTGCCCAGCGGATC
CTTATTCTTCTTGCGCATCGTGCACGGGCGCAGGAGTAAACCTGATGAAGGAAGCTACGTTTGTGTTG
CGAGGAACTATCTTGGTGAAGCAGTGAGTCGAAATGCGTCTCTGGAAGTGGCATTGTTACGAGATGAC
TTCCGACAAAACCCCACAGATGTTGTAGTGGCAGCTGGAGAGCCTGCAATCCTGGAGTGCCAGCCTCC
CCGGGGACACCCAGAACCCACCATCTACTGGAAAAAAGACAAAGTTCGAATTGATGACAAGGAAGAAA
GAATAAGTATCCGTGGTGGAAAACTGATGATCTCCAATACCAGGAAAAGTGATGCAGGGATGTATACT
TGTGTTGGTACCAATATGGTGGGAGAAAGGGACAGTGACCCAGCAGAGCTGACTGTCTTTGAACGACC
CACATTTCTCAGGAGGCCAATTAACCAGGTGGTACTGGAGGAAGAAGCTGTAGAATTTCGTTGTCAAG
TCCAAGGAGATCCTCAACCAACTGTGAGGTGGAAAAAGGATGATGCAGACTTGCCAAGAGGAAGGTAT
GACATCAAAGACGATTACACACTAAGAATTAAAAAGACCATGAGTACAGATGAAGGCACCTATATGTG
TATTGCTGAGAATCGGGTTGGAAAAATGGAAGCCTCTGCTACACTCACCGTCCGAGCTCCCCCACAGT
TTGTGGTTCGGCCAAGAGATCAGATTGTTGCTCAAGGTCGACAAGTGACATTTCCCTGTGAAACTAAA
GGAAACCCACAGCCAGCTGTTTTTTGGCAGAAAGAAGGCAGCCAGAACCTACTTTTCCCAAACCAACC
CCAGCAGCCCAACAGTAGATGCTCAGTGTCACCAACTGCAGACCTCACAATCACCAACATTCAACGTT
CCGACGCGGGTTACTACATCTGCCAGGCTTTAACTGTGGCAGGAAGCATTTTAGCAAAAGCTCAACTG
GAGGTTACTCATGTTTTGACAGATAGACCTCCACCTATAATTCTACAAGGCCCAGCCAACCAAACGCT
GGCAGTGGATCGTACAGCGTTACTGAAATGTAAAGCCACTGGTGATCCTCTTCCTGTAATTAGCTGGT
TAAAGGAGGGATTTACTTTTCCGGGTAGAGATCCAAGAGCAACAATTCAAGAGCAAGGCACACTGCAG
ATTAAGAATTTACGGATTTCTGATACTGGCACTTATACTTGTGTGGCTACAAGTTCAAGTGGAGAGAC
TTCCTGGAGTCCAGTGCTGGATGTGACAGAGTCTGGAGCAACAATCAGTAAAAACTATGATTTAAGTG
ACCTGCCAGGGCCACCATCCAAACCGCAGGTCACTGATGTTACTAAGAACAGTGTCACCTTGTCCTGG
CAGCCAGGTACCCCTGGAACCCTTCCAGCAAGTGCATATATCATTGAGGCTTTCAGCCAATCAGTCAG
CAACAGCTGGCAGACCGTGGCAAACCATGTAAAGACCACCCTCTATACTGTAAGAGGACTGCGGCCCA
ATACAATCTACTTATTCATGGTCAGAGCGATCAACCCCCAAGGTCTCAGTGACCCAAGTCCCATGTCA
GATCCTGTGCGCACACAAGATATCAGCCCACCAGCACAAGGAGTGGACCACACGCAAGTGCAGAAAGA
GCTAGGAGATGTCCTTGTCCGTCTTCATAATCCAGTTGTGCTGACTCCCACCACGGTTCAGGTCACAT
GGACGGTTGATCGCCAACCCCAGTTTATCCAAGGCTACCGAGTGATGTATCGTCAGACTTCAGGTCTG
CAGGCGACATCTTCGTGGCAGAATTTAGATGCCAAAGTCCCGACTGAACGAAGTGCTGTCTTAGTCAA
CCTGAAAAAGGGGGTGACTTATGAAATTAAAGTACGGCCATATTTTAATGAGTTCCAACGAATGGATA
GTGAATCTAAAACGGTTCGTACTACTGAAGAAGCCCCAAGTGCCCCACCACAGTCTGTCACTGTACTG
ACAGTTGGAAGCTACAATAGCACAAGTATTAGTCTTTCCTGGGATCCTCCTCCTCCAGATCACCAGAA
TGGAATTATCCAAGAATACAAGATCTGGTGTCTAGGAAATGAAACGCGATTCCATATCAACAAAACTG
TGGATGCAGCCATTCGGTCCGTAATAATTGGTGGATTATTCCCAGGTATTCAATACCGGGTAGAGGTT
GCAGCTAGTACCAGTGCAGGGGTTGGAGTAAAGAGTGAGCCACAGCCAATAATAATAGGGAGACGCAA
TGAAGTTGTCATTACTGAAAACAATAACAGCATAACTGAGCAAATCACTGATGTGGTGAAGCAACCAG
CCTTTATAGCTGGTATTGGTGGTGCCTGCTGGGTAATTCTGATGGGTTTTAGCATATGGTTGTATTGG
CGAAGAAAGAAGAGGAAGGGACTCAGTAATTATGCTTTTTCTTTTTTCATAGTTACGTTTCAAAGAGG
AGATGGAGGACTAATGAGCAATGGAAGCCGTCCAGGTCTTCTCAATGCTGGTGATCCCAGCTATCCAT
GGCTTGCTGATTCTTGGCCAGCCACGAGCTTGCCAGTAAATAATAGCAACAGTGGCCCAAATGAGATT
GGAAATTTTGGCCGTGGAGATGTGCTGCCACCAGTTCCAGGCCAAGGGGATAAAACAGCAACGATGCT
CTCAGATGGAGCCATTTATAGTAGCATTGACTTCACTACCAAAACCAGTTACAACAGTTCCAGCCAAA
TAACACAGGCTACCCCATATGCCACCACACAGATCTTGCATTCCAACAGCATACATGAATTGGCTGTC
GATCTGCCTGATCCACAATCGAAAAGCTCAATTCACCAAAAAACACATCTGATGGGATTTGGTTATTC
TCTACCTGATCAGAACAPAGGTAACAATTTACTTTACATTCCTGACTACCGATTGGCTGAGGGATTGT
CTAATAGAATGCCACACAACCAGTCTCAGGATTTCAGCACCACCAGCTCTCACAACAGCTCACAAACG
AGTGGCAGTCTTTCAGGTGGGAAAGGTGGAAAAAAGAAGAAAAATAAAAACTCTTCTAAACCACAGAA
AAACAATGCATCCACTTGGGCCAATGTCCCTCTACCTCCCCCCCCAGTCCAGCCCCTTCCTGGCACGG
AGCTGGAACACTATCCAGTGGAACAACAAGAAAATGGGTATGACAGTGATAGCTGGTGCCCACCATTG
CCAGTACAAACTTACTTACACCAAGCTCTGGAAGATGAACTGGAAGAAGATGATGATAGGGTCCCAAC
ACCTCCTGTTCGAGGCGTGCCTTCTTCTCCTGCTATCTCCTTTGGACAGCAGTCCACTGCAACTCTTA
CTCCATCCCCACGGGAAGAGATGCAACCCATGCTGCAGGCTCACCTGGATGAGTTGACAAGAGCCTAT
CAGTTTGATATAGCAAAACAAACATGGCACATTCAAAGCAATAATCAACCTCCACAGCCTCCAGTTCC
ACCGTTAGGTTATGTGTCTGGAGCCTTGATTTCTGATTTGGAAACGGATGTTGCAGATGATGATGCCG
ACGACGAAGAGGAAGCTTTAGAAATCCCCAGGCCCCTGAGACCACTGGACCAGACTCCTGGATCCAGC
ATGGACAATCTAGACAGCTCTGTGACAGGTAACGGAAGACCTCGACCTACCAGCCCATTTTCTACTGA
CAGTAACACCAGTGCAGCCCTGAGTCAAAGTCAGAGGCCTCGGCCCACTAAAAAACACAAGGGAGGGC
GGATGGACCAACAACCAGCATTCCCTCATCGAAGGGAAGGAATGACAGATGATCTTCCACCACCACCA
GATCCCCCGCCACGTCACGGTTTAAGGCAGCAAATAGGCCCGAGCCAGCAGGCTGGTAACGTGGAAAA
CTCAGCAGAGAGAAAAGGAAGCTCTCTAGAGAGACAACATGCATCCAGCTTAGAAGACACAAAGAGCT
CATTGGATTGTCCAGCTAGAACCTCCCTAGAGTGGCAGCGACAAACCCAGGAATGGATAAGCTCCACA
GAACGACAAGAAGATATACGGAAAGCCCCACACAAACAAGGTTTTTCAGAGGAGGCCTTGGTGCCCTA
TAGCAAGCCCAGTTTCCCATCTCCAGGTGGCCACAGCTCATCACGAACAGCTTCTTCTAAGGGATCCA
CTGGACCTAGGAAAACCGAGGTGTTGAGAGCAGGCCACCAGCCCAATGCCAGCGACCTTCTTGACATA
GGATATATGGGCTCCAACAGTCAAGGACAGTTTACAGGTGAATTATGTAAGTGCTTAGGTCATTTAAA
AGGCTATCGTGATTCAGAAAGAATCTTGGGTTAATAACATTGCCACATTAAACAAATTTCAGATTAAT
AGAAACTTGCTCTGTTACAAAAACAATCAATTGCAATTTTCAACAAGTTTGGTCATAA
NOV6a, CG182223-01
Protein Sequence SEQ ID NO: 72 1527 aa MW at 167842.2 kD
MSLLMFTQLLLCGFLYVRVDGSRLRQEDPPPRIVEHPSDVIVSKGEPTTLNCKAEGRPTPTIEWYKDG
ERVETDKDDPRSHRMLLPSGSLFFLRIVHGRRSKPDEGSYVCVARNYLGEAVSRNASLEVALLRDDFR
QNPTDVVVAAGEPAILECQPPRGHPEPTIYWKKDKVRIDDKEERISIRGGKLMISNTRKSDAGMYTCV
GTNMVGERDSDPAELTVFERPTFLRRPINQVVLEEEAVEFRCQVQGDPQPTVRWKKDDADLPRGRYDI
KDDYTLRIKKTMSTDECTYMCIAENRVGKMEASATLTVRAPPQFVVRPRDQIVAQGRTVTFPCETKGN
PQPAVFWQKEGSQNLLFPNQPQQPNSRCSVSPTGDLTITNIQRSDAGYYICQALTVAGSILAKAQLEV
TDVLTDRPPPIILQGPANQTLAVDGTALLKCKATGDPLPVISWLKEGFTFPGRDPRATIQEQGTLQIK
NLRISDTGTYTCVATSSSGETSWSAVLDVTESGATISKNYDLSDLPGPPSKPQVTDVTKNSVTLSWQP
GTPGTLPASAYIIEAFSQSVSNSWQTVANHVKTTLYTVRGLRPNTIYLFMVRAINPQGLSDPSPMSDP
VRTQDISPPAQGVDHRQVQKELGDVLVRLHNPVVLTPTTVQVTWTVDRQPQFIQGYRVMYRQTSGLQA
TSSWQNLDAKVPTERSAVLVNLKKGVTYEIKVRPYFNEFQCMDSESKTVRTTEEAPSAPPQSVTVLTV
GSYNSTSISVSWDPPPPDHQNGIIQEYKIWCLGNETRFHINKTVDAAIRSVIIGGLFPGIQYRVEVAA
STSAGVGVKSEPQPIIIGRRNEVVITENNNSITEQITDVVKQPAFIAGIGGACWVILMGFSIWLYWRR
KKRKGLSNYAFSFFIVTFQRGDGGLMSNGSRPGLLNAGDPSYPWLADSWPATSLPVNNSNSCPNEIGN
FGRGDVLPPVPGQGDKTATMLSDGAIYSSIDFTTKTSYNSSSQITQATPYATTQILHSNSIHELAVDL
PDPQWKSSIQQKTDLMGFGYSLPDQNKGNNLLYIPDYRLAEGLSNRMPHNQSQDFSTTSSHMSSERSG
SLSGGKGGKKKKNKNSSKPQKNNGSTWANVPLPPPPVQPLPGTELEHYAVEQQENGYDSDSWCPPLPV
QTYLHQGLEDELEEDDDRVPTPPVRGVASSPAISFGQQSTATLTPSPREEMQPMLQAHLDELTRAYQF
DIAKQTWHIQSNNQPPQPPVPPLGYVSGALISDLETDVADDDADDEEEALEIPRPLRALDQTPGSSMD
NLDSSVTGNGRPRPTSPFSTDSNTSAALSQSQRPRPTKKHKGGRMDQQPALPHRREGMTDDLPPPPDP
PPGQGLRQQIGPSQQAGNVENSAERKGSSLERQHASSLEDTKSSLDCPARTSLEWQRQTQEWISSTER
QEDIRKAPHKQGFSEEALVPYSKPSFPSPCGHSSSGTASSKGSTGPRKTEVLRAGHQRNASDLLDIGY
MGSNSQGQFTGELCKCLGHLKGYRDSERILG

Further analysis of the NOV6a protein yielded the following properties shown in Table 6B.

TABLE 6B
Protein Sequence Properties NOV6a

| SignalP | Cleavage site between residues 22 and 23 | | | | |
|---|---|---|---|---|---|
| analysis: | | | | | |
| PSORT II | PSG: a new signal peptide prediction method | | | | |
| analysis: | N-region: length 0; pos. chg 0; neg. chg 0 | | | | |
| | H-region: length 17; peak value 9.00 | | | | |
| | PSG score: 4.60 | | | | |
| | GvH: von Heijne's method for signal seq. recognition | | | | |
| | GvH score (threshold: −2.1): −3.73 | | | | |
| | possible cleavage site: between 15 and 16 | | | | |
| | >>> Seems to have no N-terminal signal peptide | | | | |
| | ALOM: Klein et al's method for TM region allocation | | | | |
| | Init position for calculation: 1 | | | | |
| | Tentative number of TMS(s) for the threshold 0.5: 2 | | | | |
| | INTEGRAL | Likelihood = | −2.81 | Transmembrane | 1-17 |
| | INTEGRAL | Likelihood = | −3.98 | Transmembrane | 860-876 |

| PERIPHERAL | Likelihood = | 1.01 (at 792) | |
|---|---|---|---|
| ALOM score: −3.98 (number of TMSs: 2) | | | |
| MTOP: Prediction of membrane topology (Hartmann et al.) | | | |
| Center position for calculation: 8 | | | |
| Charge difference: 0.0 C(1.0)-N(1.0) | | | |
| N >= C: N-terminal side will be inside | | | |
| >>> membrane topology: type 3a | | | |
| MITDISC: discrimination of mitochondrial targeting seq | | | |
| R content: | 1 | Hyd Moment (75): | 4.50 |
| Hyd Moment(95): | 2.47 | G content: | 1 |
| D/E content: | 1 | S/T content: | 2 |

```
Score: −4.61
Gavel: prediction of cleavage sites for mitochondrial preseq
R-2 motif at 28 VRV|DG
NUCDISC: discrimination of nuclear localization signals
pat4: RRKK (5) at 883
pat4: RKKR (5) at 884
pat4: KKRK (5) at 885
pat4: KKKK (5) at 1097
pat4: KKHK (3) at 1330
pat7: PTVRWKK (3) at 254
pat7: PTKKHKG (4) at 1328
bipartite: none
content of basic residues: 10.3%
NLS Score: 1.57
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: nuclear
Reliability: 94.1
COIL: Lupas's algorithm to detect coiled-coil regions
total: 0 residues
Final Results (k = 9/23):
39.1%: mitochondrial
34.8%: nuclear
17.4%: endoplasmic reticulum
  4.3%: cytoplasmic
  4.3%: peroxisomal
>> prediction for CG182223-01 is mit (k = 23)
```

A search of the NOV6a protein against the Geneseq database, a proprietary database that contains sequences published in patents and patent publication, yielded several homologous proteins shown in Table 6C.

TABLE 6C
Geneseq Results for NOV6a

| | | | Identities/ | |
|---|---|---|---|---|
| | | | Similarities for | |
| Geneseq | Protein/Organism/Length | NOV6a Residues/ | the Matched | Expect |
| Identifier | [Patent #, Date] | Match Residues | Region | Value |
| AAO19185 | Human neurotransmission- | 1 . . . 1509 | 1501/1520 (98%) | 0.0 |
| | associated protein NTRAN8 - | 1 . . . 1515 | 1501/1520 (98%) | |
| | Homo sapiens, 1515 aa. | | | |
| | [WO200266646-A2, 29 AUG. | | | |
| | 2002] | | | |
| AAO19179 | Human neurotransmission- | 1 . . . 1400 | 1356/1413 (95%) | 0.0 |
| | associated protein NTRAN2 - | 1 . . . 1405 | 1361/1413 (95%) | |
| | Homo sapiens, 1422 aa. | | | |
| | [WO200266646-A2, 29 AUG. | | | |
| | 2002] | | | |
| ABU04094 | Human expressed protein tag | 21 . . . 1495 | 819/1610 (50%) | 0.0 |
| | (EPT) #760 - Homo sapiens, 1651 | 58 . . . 1634 | 1040/1610 (63%) | |
| | aa. [WO200278524-A2, 10 OCT. | | | |
| | 2002] | | | |
| ABU04093 | Human expressed protein tag | 21 . . . 1495 | 819/1610 (50%) | 0.0 |
| | (EPT) #759 - Homo sapiens, 1651 | 58 . . . 1634 | 1040/1610 (63%) | |
| | aa. [WO200278524-A2, 10 OCT. | | | |
| | 2002] | | | |
| ABU04092 | Human expressed protein tag | 21 . . . 1495 | 819/1610 (50%) | 0.0 |
| | (EPT) #758 - Homo sapiens, 1651 | 58 . . . 1634 | 1040/1610 (63%) | |
| | aa. [WO200278524-A2, 10 OCT. | | | |
| | 2002] | | | |

In a BLAST search of public sequence databases, the NOV6a protein was found to have homology to the proteins shown in the BLASTP data in Table 6D.

TABLE 6D
Public BLASTP Results for NOV6a

| | | | Identities/ | |
|---|---|---|---|---|
| Protein | | | Similarities for | |
| Accession | | NOV6a Residues/ | the Matched | Expect |
| Number | Protein/Organism/Length | Match Residues | Portion | Value |
| Q9HCK4 | Hypothetical protein KIAA1568 - | 1 . . . 1400 | 1316/1408 (93%) | 0.0 |
| | Homo sapiens (Human), 1380 aa | 3 . . . 1361 | 1321/1408 (93%) | |
| | (fragment). | | | |
| Q90Z70 | Roundabout2 - Brachydanio rerio | 7 . . . 1509 | 1152/1525 (75%) | 0.0 |
| | (Zebrafish) (Danio rerio), 1513 aa. | 5 . . . 1513 | 1294/1525 (84%) | |
| Q9QZI3 | Robo2 - Rattus norvegicus (Rat), | 1 . . . 1053 | 974/1056 (92%) | 0.0 |
| | 1060 aa (fragment). | 1 . . . 1050 | 1001/1056 (94%) | |
| Q8UVD7 | Roundabout-1 - Xenopus laevis | 10 . . . 1495 | 826/1620 (50%) | 0.0 |
| | (African clawed frog), 1614 aa. | 10 . . . 1598 | 1060/1620 (64%) | |
| Q9Y6N7 | Roundabout 1 - Homo sapiens | 21 . . . 1495 | 819/1610 (50%) | 0.0 |
| | (Human), 1651 aa. | 58 . . . 1634 | 1040/1610 (63%) | |

PFam analysis predicts that the NOV6a protein contains the domains shown in the Table 6E.

TABLE 6E
Domain Analysis of NOV6a

| | | Identities/ | |
|---|---|---|---|
| | NOV6a | Similarities for | |
| Pfam | Match | the Matched | Expect |
| Domain | Region | Region | Value |
| ig | 45 . . . 112 | 17/71 (24%) | 6.2e−06 |
| | | 52/71 (73%) | |
| ig | 147 . . . 205 | 16/61 (26%) | 8.2e−06 |
| | | 42/61 (69%) | |
| ig | 239 . . . 295 | 17/60 (28%) | 1.6e−08 |
| | | 44/60 (73%) | |
| ig | 328 . . . 393 | 17/69 (25%) | 7.6e−09 |
| | | 51/69 (74%) | |
| ig | 432 . . . 490 | 17/62 (27%) | 1.8e−08 |
| | | 46/62 (74%) | |
| fn3 | 522 . . . 607 | 33/88 (38%) | 3.2e−17 |
| | | 64/88 (73%) | |
| fn3 | 638 . . . 724 | 24/90 (27%) | 0.0086 |
| | | 63/90 (70%) | |
| fn3 | 736 . . . 826 | 33/93 (35%) | 3.5e−14 |
| | | 64/93 (69%) | |

### Example 7

The NOV7 clone was analyzed, and the nucleotide and encoded polypeptide sequences are shown in Table 7A.

TABLE 7A
NOV7 Sequence Analysis

NOV7a, CG183585-01 SEQ ID NO: 73 1385 bp
DNA Sequence ORF Start: ATG at 145 ORF Stop: TAG at 1264
CTTGTATATATAATGGTAGACTGTAAAGGGTACCTTCCCCACCTGATATTCTGGAATGTCAGTTTGTA
GGTCGACATTGCGACTTCTTTTTCCTTAGCAGAGCCAAGCTCCATTCAGCTGGTTACCACTTTGTGGG
TGTCTTTAATGAAGCTTATAAATGGCAGGAAGCAAACATTCCCGTGGTTTGGCATGGATATTCGTGGA
ACCCTGGTTAAGTTGGTTTACTTTGAACCGAAGGATATCACGGCAGAAGAAGAGCAGGAAGAAGTGGA
GAACCTGAAGAGCATCCGGAACTATTTGACTTCTAATACTGCTTATGGGAAAACTGGGATCCGAGACG
TCCACCTGGAACTGAAAAACCTGACCATGTGTGGACGCAAAGGGAACCTGCACTTCATCCGCTTTCCC
AGCTGTGCCATGCACAGGTTCATTCAGATGCGCAGCGAGAAGAACTTCTCTAGCCTTCACACCACCCT
CTCTGCCACAGGAGGCGGGGCTTTCAAATTCGAAGACGACTTCAGAATGATTGCTGACCTGCAGCTCC
ATAAACTGGATGAACTGGACTGTCTGATTCAGGGCCTGCTTTATGTCGACTCTGTTGGCTTCAACGGC
AAGCCAGAATGTTACTATTTTGAAAATCCCACAAATCCTGAATTGTGTCAAAAAAAGCCGTACTGCCT
TGATAACCCATACCCTATGTTGCTGGTTAACATGGGCTCAGGTGTCAGCATTCTAGCCGTGTACTCCA
AGGACAACTATAAAAGAGTTACAGGCACCAGTCTTGGAGGTGGAACATTCCTAGGCCTATGTTGCTTG
CTGACTGGTTGTGAGACCTTTGAAGAAGCTCTGGAAATGGCAGCTAAAGGCGACAGCACCAATGTTGA
TAAACTGGTGAAGGACATTTACGGAGGAGACTATGAACGATTTGGCCTTCAAGGATCTGCTGTAGCAT
CAAGCTTGGGCAACATGATGAGTAAAGAAAAGCGAGATTCCATCAGCAAGGAAGACCTCGCCCGGCCC
ACATTGGTCACCATCACCAACAACATTGGCTCCATTGCTCGCATGTGTGCGTTGAATGAGAACATAGA
CAGAGTTGTGTTTGTTGGAAATTTTCTCAGAATCAATATGGTCTCCATGAAGCTGCTGGCATATGCCA
TGGATTTTTGGTCCAAACGACAACTGAAAGCTCTGTTTTTGGAACATCAGGGTTATTTTGGAGCCGTT
GGGGCACTGTTGGAACTGTTCAAAATGACTGATGATAAGTAGAGACGAGCAGTGGAGGAAACAGCCTC
CCATTATGGCAGATGAACCTGCTGG
NOV7a, CG183585-01
Protein Sequence SEQ ID NO: 74 373 aa MW at 41664.6 kD
MKLINGRKQTFPWFGMDIGGTLVKLVYFEPKDITAEEEQEEVENLKSIRKYLTSNTAYGKTGIRDVHL
ELKNLTMCGRKGNLHFIRFPSCAMHRFIQMGSEKNFSSLHTTLCATGGGAFKFEEDFRMIADLQLHKL
DELDCLIQGLLYVDSVGFNGKPECYYFENPTNPELCQKKPYCLDNPYPMLLVNMGSGVSILAVYSKDN
YKRVTGTSLGGGTFLGLCCLLTGCETFEEALEMAAKGDSTNVDKLVKDIYGGDYERFGLQGSAVASSL
GNMMSKEKRDSISKEDLARATLVTITNNIGSIARMCALNENIDRVVFVGNFLRINMVSMKLLAYAMDF
WSKGQLKALFLEHEGYFGAVGALLELFKMTDDK

Further analysis of the NOV7a protein yielded the following properties shown in Table 7B.

TABLE 7B
Protein Sequence Properties NOV7a

| SignalP | No Known Signal Sequence Predicted | | | |
|---|---|---|---|---|
| analysis: | | | | |
| PSORT II | PSG: a new signal peptide prediction method | | | |
| analysis: | N-region: length 8; pos. chg 3; neg. chg 0 | | | |
| | H-region: length 8; peak value 5.54 | | | |
| | PSG score: 1.14 | | | |
| | GvH: von Heijne's method for signal seq. recognition | | | |
| | GvH score (threshold: −2.1): −10.98 | | | |
| | possible cleavage site: between 59 and 60 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 1 | | | |
| | Number of TMS(s) for threshold 0.5: 0 | | | |
| | PERIPHERAL Likelihood = 0.95 (at 212) | | | |
| | ALOM score: 0.42 (number of TMSs: 0) | | | |
| | MTOP: Prediction of membrane topology (Hartmann et al.) | | | |
| | Center position for calculation: 6 | | | |
| | Charge difference: −2.0 C(1.0)-N(3.0) | | | |
| | N >= C: N-terminal side will be inside | | | |
| | MITDISC: discrimination of mitochondrial targeting seq | | | |
| | R content: | 1 | Hyd Moment(75): | 10.35 |
| | Hyd Moment(95): | 1.52 | G content: | 2 |
| | D/E content: | 1 | S/T content: | 1 |
| | Score: | −4.69 | | |

Gavel: prediction of cleavage sites for mitochondrial preseq
R-2 motif at 17 GRK|QT
NUCDISC: discrimination of nuclear localization signals
pat4: none
pat7: none
bipartite: none
content of basic residues: 11.3%
NLS Score: −0.47
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 94.1
COIL: Lupas's algorithm to detect coiled-coil regions
total: 0 residues
Final Results (k = 9/23):
60.9%: cytoplasmic
21.7%: nuclear
17.4%: mitochondrial
>> prediction for CG183585-01 is cyt (k = 23)

A search of the NOV7a protein against the Geneseq database, a proprietary database that contains sequences published in patents and patent publication, yielded several homologous proteins shown in Table 7C.

TABLE 7C
Geneseq Results for NOV7a

| | | | Identities/ | |
|---|---|---|---|---|
| | | | Similarities for | |
| Geneseq | Protein/Organism/Length | NOV7a Residues/ | the Matched | Expect |
| Identifier | [Patent #, Date] | Match Residues | Region | Value |
| AAE24134 | Human kinase (PKIN)-5 protein - | 1 . . . 373 | 371/373 (99%) | 0.0 |
| | Homo sapiens, 373 aa. | 1 . . . 373 | 372/373 (99%) | |
| | [WO200233099-A2, 25 APR. 2002] | | | |
| AAE21720 | Human PKIN-15 protein - Homo | 7 . . . 369 | 297/363 (81%) | e−178 |
| | sapiens, 447 aa. [WO200218557- | 84 . . . 446 | 332/363 (90%) | |
| | A2, 07 MAR. 2002] | | | |
| AAM40613 | Human polypeptide SEQ ID NO | 7 . . . 369 | 297/363 (81%) | e−178 |
| | 5544 - Homo sapiens, 460 aa. | 97 . . . 459 | 332/363 (90%) | |
| | [WO200153312-A1, 26 JUL. 2001] | | | |
| AAM38827 | Human polypeptide SEQ ID NO | 7 . . . 369 | 296/363 (81%) | e−178 |
| | 1972 - Homo sapiens, 447 aa. | 84 . . . 446 | 331/363 (90%) | |
| | [WO200153312-A1, 26 JUL. 2001] | | | |
| AAB94366 | Human protein sequence SEQ ID | 1 . . . 366 | 291/366 (79%) | e−173 |
| | NO: 14899 - Homo sapiens, 370 aa. | 1 . . . 366 | 330/366 (89%) | |
| | [EP1074617-A2, 07 FEB. 2001] | | | |

In a BLAST search of public sequence databases, the NOV7a protein was found to have homology to the proteins shown in the BLASTP data in Table 7D.

TABLE 7D
Public BLASTP Results for NOV7a

| | | | Identities/ | |
|---|---|---|---|---|
| Protein | | | Similarities for | |
| Accession | | NOV7a Residues/ | the Matched | Expect |
| Number | Protein/Organism/Length | Match Residues | Portion | Value |
| BAC34132 | Adult male liver tumor cDNA, RIKEN | 1 . . . 371 | 365/371 (98%) | 0.0 |
| | full-length enriched library, | 1 . . . 371 | 369/371 (99%) | |
| | clone: C730027O17 | | | |
| | product: hypothetical protein, full | | | |
| | insert sequence - Mus musculus | | | |
| | (Mouse), 373 aa. | | | |
| Q8TE04 | Pantothenate kinase 1 (EC 2.7.1.33) | 2 . . . 373 | 365/374 (97%) | 0.0 |
| | (Pantothenic acid kinase 1) (hPanK1) | 225 . . . 598 | 368/374 (97%) | |
| | (hPanK) - Homo sapiens (Human), | | | |
| | 598 aa. | | | |
| Q8K4K6 | Pantothenate kinase 1 (EC 2.7.1.33) | 2 . . . 371 | 359/372 (96%) | 0.0 |
| | (Pantothenic acid kinase 1) (mPank1) | 175 . . . 546 | 365/372 (97%) | |
| | (mPank) - Mus musculus (Mouse), | | | |
| | 548 aa. | | | |
| Q9BZ23 | Pantothenate kinase 2 (EC 2.7.1.33) | 7 . . . 369 | 297/363 (81%) | e−178 |
| | (Pantothenic acid kinase 2) (hPANK2) - | 207 . . . 569 | 332/363 (90%) | |
| | Homo sapiens (Human), 570 aa. | | | |
| Q9H999 | Pantothenate kinase 3 (EC 2.7.1.33) | 1 . . . 366 | 291/366 (79%) | e−173 |
| | (Pantothenic acid kinase 3) (hPanK3) - | 1 . . . 366 | 330/366 (89%) | |
| | Homo sapiens (Human), 370 aa. | | | |

PFam analysis predicts that the NOV7a protein contains the domains shown in the Table 7E.

TABLE 7E
Domain Analysis of NOV7a

| | | Identities/ | |
|---|---|---|---|
| | NOV7a | Similarities for | |
| Pfam | Match | the Matched | Expect |
| Domain | Region | Region | Value |
| Fumble | 12 . . . 367 | 196/401 (49%) | 2.3e−234 |
| | | 346/401 (86%) | |

### Example 8

The NOV8 clone was analyzed, and the nucleotide and encoded polypeptide sequences are shown in Table 8A.

TABLE 8A
NOV8 Sequence Analysis

NOV8a, CG183860-01 SEQ ID NO: 75 1858 bp
DNA Sequence ORF Start: ATG at 72 ORF Stop: TGA at 786
CAGGTAGCCCGGCTCAGCCCTTCGCTTTCCAGCTGCGTCCTGCTCCCGGCCGCCCAGGGAGCCCACTG
GCGATGAGGGCACTGCTGGCGCTTTGCCTTCTCCTTGGCTGGCTGCGCTGGGGCCCGGCGGGCGCCCA
GCAGTCCGGACAGTACTGCCACGGCTGGGTGGACGTGCAGGGCAACTACCACCAGGGCTTCCAGTGCC
CAGAGGACTTCGACACGCTGGACGCTACCATCTCCTGCGGCTCCTGCGCGCTCCGCTACTGTTGCGCC
GCGGTCGACGCCAGGCTCGAGCAGGGCGGCTGCACCAACGACCGCCGCGAACTGGAGCACCCAGGCAT
CACTGCGCAGCCTUTCTACGTCCCCTTTCTCATCGTCGCCTCCATCTTCATTGCGTTCATCATCCTGG
GCTCTGTAGTGGCTATTTATTGTTGCACCTGTTTGAGACCCAAGGAGCCCTCGCAGCAGCCAATCCGC
TTCTCACTCCGCAGCTATCAGACAGAGACCCTGCCCATGATCCTGACCTCCACCAGCCCCAGGGCACC
CTCCCGGCAGTCCAGCACAGCCACGAGCTCCAGCTCCACAGCCGGCTCCATCCGCAGGTTCTCCTTTG
CCAGGGCTGAGCCGGGCTGCCTGCTGCCCTCACCGCCCCCGCCATACACCACCAGCCACTCAATCCAC
CTGGCTCAGCCATCTGGTTTCCTGGTGTCACCCCAGTATTTCGCTTACCCCCTCCAGCAGGAGCCCCC
ACTGCCTGGGAAGAGCTGTCCAGACTTCAGTTCCAGTTGACACGCCCAGGCCATGAATCCACAACTCA
GTCAGATGGCACACAGGTGGAGCCCTGCTCCCATTGCCACATGCAATTCTGAGAAAATTTCCCTTGTA
ACTGATCAGTGTCATGGAGGAGCATGCTAGGAAAACACAGCACCTTCTAATTTGAAAGTTCCTGTCTC
CAATCACAGAAAGGCTAAACCAGAGAACTGTTTTCTGGTTTTGCAAACATGTGATCATTACATTTCAA
TCTATGCTACTTTTATTCAAAATATGCAGCAGTTTGACTTTAAAGTTGCAAACTGGCTAAAAACGTTT
TACTGGACATTCAGCTATATTGCTTAGAAAAGGGCTACATGTTTCTTTTTCATATAAGTTGTTCATTG
AGTTATGATAGGAATATATTCATAAATAAGCAAAGAAAAATACCTAATTGTAATTATCAAACGTTCAC
TTAAAAAAATTAACTATTAGGTAAACTTAAGCGGGCAGTGAAAAATCTATTTATGATTTCGGGAGTAA
CCTAACCATGAATAATATTAGCATAATGAGAACATTTACTTTTTAAATAAATAACTAAATTTTGTTTA
CAATATGAGTTTTTCCAGAATACAAGGTTTCAATAATCACATGAGGAGTTTAAAGTTTTAAATATATA
CTCAGACATTCATTGTAACACAGAGTGTATGTAAAATCATTTCCCCCACTCACTGGAGGGAGTATTTA
TTGCACACTTTTTGTTCAGCAACATTTAGTGTTTCACTGAAAGTTGGACAGTTGGGGCTTAAAACATT
TATTTGTAAAATGAGCTATGTTCAAATGTAAATATTTGTAAATTTATGTATTTACCACATTGACTGTA
CTAATTATTTAGTAGTCATACTGTAATTTTTATGTTAATAATAACTGGAGTTCAAAGTCTAGCTATTG
GTATAATCATCTAATATTATATATATCTCCAGTGCCCCTGAATTTTATGTTTGATCACTATATATTTG
GGCATATATCTTGTTGGATTAGAATAAATAAAACACTTTATATTTTCATGAACTCTAAAAAAAAAAAA
AAAAAAAAAAAAAAAAAAAGGG
NOV8a, CG183860-01
Protein Sequence SEQ ID NO: 76 238 aa Mw at 25860.1 kD
MRALLALCLLLGWLRWGPAGAQQSGEYCHGWVDVQGNYHEGFQCPEDFDTLDATICCGSCALRYCCAA
VDARLEQGGCTNDRRELEHPGITAQPVYVPFLIVGSIFIAFIILGSVVAIYCCTCLRPKEPSQQPIRF
SLRSYQTETLPMILTSTSPRAPSRQSSTATSSSSTGGSIRRFSFAPAEPGCLVPSPPPPYTTSHSIHL
AQPSGFLVSPQYFAYPLQQEPPLPGKSCPDFSSS

Further analysis of the NOV8a protein yielded the following properties shown in Table 8B.

TABLE 8B
Protein Sequence Properties NOV8a

| SignalP | Cleavage site between residues 22 and 23 | | | | |
|---|---|---|---|---|---|
| analysis: | | | | | |
| PSORT II | PSG: a new signal peptide prediction method | | | | |
| analysis: | N-region: length 2; pos. chg 1; neg. chg 0 | | | | |
| | H-region: length 12; peak value 10.66 | | | | |
| | PSG score: 6.26 | | | | |
| | GvH: von Heijne's method for signal seq. recognition | | | | |
| | GvH score (threshold: −2.1): 1.27 | | | | |
| | possible cleavage site: between 21 and 22 | | | | |
| | >>> Seems to have a cleavable signal peptide (1 to 21) | | | | |
| | ALOM: Klein et al's method for TM region allocation | | | | |
| | Init position for calculation: 22 | | | | |
| | Tentative number of TMS(s) for the threshold 0.5: 1 | | | | |
| | Number of TMS(s) for threshold 0.5: 1 | | | | |
| | INTEGRAL | Likelihood = | −11.04 | Transmembrane | 100-116 |

| PERIPHERAL | Likelihood = | 1.27 (at 53) | |
|---|---|---|---|
| ALOM score: −11.04 (number of TMSs: 1) | | | |
| MTOP: Prediction of membrane topology (Hartmann et al.) | | | |
| Center position for calculation: 10 | | | |
| Charge difference: −1.5 C(0.5)-N(2.0) | | | |
| N >= C: N-terminal side will be inside | | | |
| >>> membrane topology: type 1a (cytoplasmic tail 117 to 238) | | | |
| MITDISC: discrimination of mitochondrial targeting seq | | | |
| R content: | 2 | Hyd Moment(75): | 6.25 |
| Hyd Moment(95): | 8.60 | G content: | 4 |
| D/E content: | 1 | S/T content: | 1 |

Score: −4.46
Gavel: prediction of cleavage sites for mitochondrial preseq
R-2 motif at 25 LRW|GP
NUCDISC: discrimination of nuclear localization signals
pat4: none
pat7: none
bipartite: none
content of basic residues: 6.7%
NLS Score: −0.47
KDEL: ER retention motif in the C-terminus: none
ER Membrane Retention Signals:
XXRR-like motif in the N-terminus: RALL
none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: too long tail
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: nuclear
Reliability: 89
COIL: Lupas's algorithm to detect coiled-coil regions
total: 0 residues
Final Results (k = 9/23):
44.4%: extracellular, including cell wall
22.2%: Golgi
22.2%: endoplasmic reticulum
11.1%: plasma membrane
>> prediction for CG183860-01 is exc (k = 9)

A search of the NOV8a protein against the Geneseq database, a proprietary database that contains sequences published in patents and patent publication, yielded several homologous proteins shown in Table 8C.

TABLE 8C
Geneseq Results for NOV8a

| | | NOV8a | Identities/ | |
|---|---|---|---|---|
| | | Residues/ | Similarities for | |
| Geneseq | Protein/Organism/Length [Patent | Match | the Matched | Expect |
| Identifier | #, Date] | Residues | Region | Value |
| AAY86234 | Human secreted protein HNTNC20, | 1 . . . 195 | 179/195 (91%) | e−103 |
| | SEQ ID NO: 149 - Homo sapiens, 219 | 1 . . . 195 | 179/195 (91%) | |
| | aa. [WO9966041-A1, 23 DEC. 1999] | | | |
| ABU56619 | Lung cancer-associated polypeptide | 19 . . . 212 | 107/204 (52%) | 2e−51 |
| | #212 - Unidentified, 295 aa. | 29 . . . 226 | 136/204 (66%) | |
| | [WO200286443-A2, 31 OCT. 2002] | | | |
| ABB85001 | Human PRO28631 protein sequence | 19 . . . 212 | 107/204 (52%) | 2e−51 |
| | SEQ ID NO: 370 - Homo sapiens, 295 | 29 . . . 226 | 136/204 (66%) | |
| | aa. [WO200200690-A2, 03 JAN. | | | |
| | 2002] | | | |
| ABB95607 | Human angiogenesis related protein | 19 . . . 212 | 107/204 (52%) | 2e−51 |
| | PRO28631 SEQ ID NO: 370 - Homo | 29 . . . 226 | 136/204 (66%) | |
| | sapiens, 295 aa. [WO200208284-A2, | | | |
| | 31 JAN. 2002] | | | |
| ABG61896 | Prostate cancer-associated protein | 13 . . . 84 | 48/72 (66%) | 3e−25 |
| | #97 - Mammalia, 582 aa. | 243 . . . 314 | 56/72 (77%) | |
| | [WO200230268-A2, 18 APR. 2002] | | | |

In a BLAST search of public sequence databases, the NOV8a protein was found to have homology to the proteins shown in the BLASTP data in Table 8D.

TABLE 8D
Public BLASTP Results for NOV8a

| | | NOV8a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q8C5V3 | Hypothetical protein - Mus | 79 . . . 238 | 149/160 (93%) | 4e−83 |
| | musculus (Mouse), 160 aa | 1 . . . 160 | 153/160 (95%) | |
| | (fragment). | | | |
| Q96EQ5 | Hypothetical protein - Homo | 96 . . . 238 | 143/143 (100%) | 3e−79 |
| | sapiens (Human), 144 aa | 2 . . . 144 | 143/143 (100%) | |
| | (fragment). | | | |
| Q8QZV2 | Hypothetical protein - Mus | 2 . . . 212 | 114/221 (51%) | 1e−51 |
| | musculus (Mouse), 295 aa. | 15 . . . 226 | 142/221 (63%) | |
| Q8BN61 | Hypothetical protein - Mus | 2 . . . 212 | 113/221 (51%) | 1e−50 |
| | musculus (Mouse), 295 aa. | 15 . . . 226 | 141/221 (63%) | |
| CAC51150 | Sequence 26 from Patent | 24 . . . 196 | 44/183 (24%) | 6e−06 |
| | WO0149728 - Homo sapiens | 27 . . . 187 | 76/183 (41%) | |
| | (Human), 197 aa. | | | |

### Example 9

The NOV9 clone was analyzed, and the nucleotide and encoded polypeptide sequences are shown in Table 9A.

TABLE 9A
NOV9 Sequence Analysis

NOV9a, CG184416-01```SEQ ID NO: 77``````````` 1321 bp
DNA Sequence ORF Start: ATG at 39 ORF Stop: TGA at 1284
CTGCCCCATGCAGCCCTGAGCCCCACAGCAAGTCTGCCATGGGCCGCGGGGCCCGTGTCCCCTCGGAG
GCCCCGGGGGCAGGCGTCGAGCGCCGCTGGCTTGGAGCCGCGCTGGTCGCCCTGTGCCTCCTCCCCGC
GCTGGTGCTGCTGGCCCCGCTGGGGGCCCCGGCGGTGCCGGCCTGGAGCGCAGCGCAGGGAGACGTCG
CTGCGCTGGGCCTCTCGGCGGTCCCCCCCACCCGGGTCCCGGGCCCACTGGCCCCCCGCAGACGCCGC
TACACGCTGACTCCAGCCAGGCTGCGCTGCGACCACTTCAACCTCACCTACAGGATCCTCTCCTTCCC
GCGGAACCTGCTGAGCCCGCGGGAGACGCGGCGGGCCCTAGCTGCCGCCTTCCGCATGTGGAGCGACG
TGTCCCCCTTCAGCTTCCGCGAGGTGGCCCCCGAGCAGCCCAGCGACCTCCGGATAGGCTTCTACCCG
ATCAACCACACCCACTGCCTGGTCTCCGCGCTGCACCACTGCTTCGACCGCCCCACGGGGGAGCTGGC
CCACGCCTTCTTCCCCCCGCACGGCGGCATCCACTTCGACGACAGCGAGTACTGGGTCCTGGGCCCCA
CGCGCTACAGCTGGAAGAAAGGCGTGTGGCTCACGGACCTGGTGCACGTGGCGGCCCACGAGATCGGC
CACGCGCTGGGCCTGATGCACTCACAACACGGCCGGGCGCTCATGCACCTGAACGCCACGCTGCGCGG
CTGGAAGGCGTTGTCCCAGGACGAGCTGTGGGGCCTGCACCGGCTCTACGGTGAGTCCCTTTGTCGGG
CGGGAGGGCGCCCACCGGGCGGTCCTGAGCCAGGCGTGCTCCCCACGCTCCCGATAGGATGCCTCGAC
AGGCTGTTCGTGTGCGCGTCCTGGGCGCGGAGGCGCTTCTGCGACGCTCGCCGGCGGCTCATGAAGAG
GCTCTGCCCCAGCAGCTCCGACTTCTGCTACGAATTCCCCTTCCCCACGGTGGCCACCACCCCACCGC
CCCCCAGGACCAAAACCAGGCTGGTGCCCGAGGGCAGGAACGTGACCTTCCGCTCCGGCCAGAAGATC
CTCCACAAGAAAGGGAAAGTGTACTGGTACAAGGACCAGGAGCCCCTGGAGTTCTCCTACCCCGGCTA
CCTGGCCCTGGGCGAGGCGCACCTGAGCATCATCGCCAACGCCGTCAATGAGGGCACCTACACCTGCG
TGGTGCGCCGCCAGCAGCGCGTGCTGACCACCTACTCCTGGCGAGTCCGTGTGCGGGGCTGAGCCCGG
CTGATAAAGCACTTTCTCTCTGAAAAAAAAAA
NOV9a, CG184416-01
Protein Sequence SEQ ID NO: 78 415 aa MW 46304.0 kD
MGRGARVPSEAPGAGVERRWLGAALVALCLLPALVLLARLGAPAVPAWSAAQGDVAALGLSAVPPTRV
PGPLAPRRRRYTLTPARLRWDHFNLTYRILSFPRNLLSPRETRRALAAAFRMWSDVSPFSFREVAPEQ
PSDLRIGFYPINHTDCLVSALHHCFDGPTGELAHAFFPPHGGIHFDDSEYWVLGPTRYSWKKGVWLTD
LVHVAAHEIGHALGLMHSQHGRALMHLNATLRGWKALSQDELWGLHRLYGESLCRAGGRGPGGPEPGV
LPTLPIGCLDRLFVCASWARRGFCDARRRLMKRLCPSSCDFCYEFPFPTVATTPPPPRTKTRLVPEGR
NVTFRCGQKILHKKGKVYWYKDQEPLEFSYPGYLALGEAHLSIIANAVNEGTYTCVVRRQQRVLTTYS
WRVRVRG

Further analysis of the NOV9a protein yielded the following properties shown in Table 9B.

TABLE 9B
Protein Sequence Properties NOV9a

| SignalP | Cleavage site between residues 45 and 46 | | | | |
|---|---|---|---|---|---|
| analysis: | | | | | |
| PSORT II | PSG: a new signal peptide prediction method | | | | |
| analysis: | N-region: length 10; pos. chg 2; neg. chg 1 | | | | |
| | H-region: length 6; peak value −6.74 | | | | |
| | PSG score: −11.14 | | | | |
| | GvH: von Heijne's method for signal seq. recognition | | | | |
| | GvH score (threshold: −2.1): 1.31 | | | | |
| | possible cleavage site: between 37 and 38 | | | | |
| | >>> Seems to have no N-terminal signal peptide | | | | |
| | ALOM: Klein et al's method for TM region allocation | | | | |
| | Init position for calculation: 1 | | | | |
| | Tentative number of TMS(s) for the threshold 0.5: 1 | | | | |
| | Number of TMS(s) for threshold 0.5: 1 | | | | |
| | INTEGRAL | Likelihood = | −10.40 | Transmembrane | 21-37 |
| | PERIPHERAL | Likelihood = | 0.79 (at 272) | | |

| ALOM score: −10.40 (number of TMSs: 1) | | | |
|---|---|---|---|
| MTOP: Prediction of membrane topology (Hartmann et al.) | | | |
| Center position for calculation: 28 | | | |
| Charge difference: 0.0 C(1.0)-N(1.0) | | | |
| N >= C: N-terminal side will be inside | | | |
| >>> membrane topology: type 2 (cytoplasmic tail 1 to 21) | | | |
| MITDISC: discrimination of mitochondrial targeting seq | | | |
| R content: | 2 | Hyd Moment (75): | 4.37 |
| Hyd Moment (95): | 11.61 | G content: | 4 |
| D/E content: | 2 | S/T content: | 1 |

```
Score: −6.42
Gavel: prediction of cleavage sites for mitochondrial preseq
R-2 motif at 16 ARV|PS
NUCDISC: discrimination of nuclear localization signals
pat4: PRRR (4) at 74
pat4: RRRR (5) at 75
pat7: PLAPRRR (3) at 71
pat7: PRRRRYT (5) at 74
bipartite: RRQQRVLTTYSWRVRVR at 398
content of basic residues: 12.8%
NLS Score: 1.27
KDEL: ER retention motif in the C-terminus: none
ER Membrane Retention Signals:
XXRR-like motif in the N-terminus: GRGA
none
SKL: peroxisomal targeting signal in the C-terminus: none
PTS2: 2nd peroxisomal targeting signal: none
VAC: possible vacuolar targeting motif: found
TLPI at 275
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 94.1
COIL: Lupas's algorithm to detect coiled-coil regions
total: 0 residues
Final Results (k = 9/23):
39.1%: mitochondrial
30.4%: cytoplasmic
  8.7%: vacuolar
  8.7%: endoplasmic reticulum
  4.3%: Golgi
  4.3%: vesicles of secretory system
  4.3%: nuclear
>> prediction for CG184416-01 is mit (k = 23)
```

A search of the NOV9a protein against the Geneseq database, a proprietary database that contains sequences published in patents and patent publication, yielded several homologous proteins shown in Table 9C.

TABLE 9C
Geneseq Results for NOV9a

| | | NOV9a | Identities/ | |
|---|---|---|---|---|
| | | Residues/ | Similarities for | |
| Geneseq | Protein/Organism/Length [Patent | Match | the Matched | Expect |
| Identifier | #, Date] | Residues | Region | Value |
| ABG72777 | Human matrix metalloproteinase 23 | 1 . . . 415 | 390/415 (93%) | 0.0 |
| | (MMP23) protein - Homo sapiens, | 1 . . . 390 | 390/415 (93%) | |
| | 390 aa. [WO200285285-A2, 31 OCT. | | | |
| | 2002] | | | |
| AAB84622 | Amino acid sequence of matrix | 1 . . . 415 | 390/415 (93%) | 0.0 |
| | metalloproteinase-21 - Homo sapiens, | 1 . . . 390 | 390/415 (93%) | |
| | 390 aa. [WO200149309-A2, 12 JUL. | | | |
| | 2001] | | | |
| AAE10430 | Human matrix metalloprotinase-22P | 1 . . . 415 | 390/415 (93%) | 0.0 |
| | (MMP-22P) protein - Homo sapiens, | 1 . . . 390 | 390/415 (93%) | |
| | 390 aa. [WO200166766-A2, 13 SEP. | | | |
| | 2001] | | | |
| AAY78585 | Metalloprotease in the female | 1 . . . 415 | 390/415 (93%) | 0.0 |
| | reproductive tract protein sequence - | 1 . . . 390 | 390/415 (93%) | |
| | Homo sapiens, 390 aa. | | | |
| | [JP2000014387-A, 18 JAN. 2000] | | | |
| AAY78353 | Rat metalloproteinase protein | 1 . . . 414 | 327/417 (78%) | 0.0 |
| | sequence SEQ ID NO: 2 - Rattus | 1 . . . 390 | 344/417 (82%) | |
| | norvegicus, 391 aa. [JP2000014386- | | | |
| | A, 18 JAN. 2000] | | | |

In a BLAST search of public sequence databases, the NOV9a protein was found to have homology to the proteins shown in the BLASTP data in Table 9D.

TABLE 9D
Public BLASTP Results for NOV9a

| | | NOV9a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9UBR9 | MMP-23 (MIFR/FEMALYSIN) | 1 . . . 415 | 390/415 (93%) | 0.0 |
| | (DJ283E3.2.1) (Matrix | 1 . . . 390 | 390/415 (93%) | |
| | metalloproteinase MMP21/22A | | | |
| | (MIFR1)) (Matrix metalloproteinase | | | |
| | 23B) - Homo sapiens (Human), 390 | | | |
| | aa. | | | |
| O75900 | Metalloprotease mmp21/22A - Homo | 1 . . . 415 | 389/415 (93%) | 0.0 |
| | sapiens (Human), 390 aa. | 1 . . . 390 | 389/415 (93%) | |
| O88676 | cAMP metalloproteinase - Mus | 1 . . . 414 | 328/416 (78%) | 0.0 |
| | musculus (Mouse), 391 aa. | 1 . . . 390 | 345/416 (82%) | |
| O88272 | MIFR - Rattus norvegicus (Rat), 391 | 1 . . . 414 | 327/417 (78%) | 0.0 |
| | aa. | 1 . . . 390 | 344/417 (82%) | |
| O75894 | Metalloprotease isoform C | 149 . . . 398 | 250/250 (100%) | e−156 |
| | (Metalloprotease MMP21/22C) - | 1 . . . 250 | 250/250 (100%) | |
| | Homo sapiens (Human), 250 aa | | | |
| | (fragment). | | | |

PFam analysis predicts that the NOV9a protein contains the domains shown in the Table 9E.

TABLE 9E
Domain Analysis of NOV9a

| | | Identities/ | |
|---|---|---|---|
| | NOV9a | Similarities | |
| Pfam | Match | for the | Expect |
| Domain | Region | Matched Region | Value |
| Peptidase_M10 | 81 . . . 192 | 43/115 (37%) | 1.9e−23 |
| | | 74/115 (64%) | |
| ShTK | 279 . . . 315 | 16/44 (36%) | 3.4e−09 |
| | | 27/44 (61%) | |
| ig | 339 . . . 397 | 17/61 (28%) | 0.00051 |
| | | 39/61 (64%) | |

### Example 10

The NOV10 clone was analyzed, and the nucleotide and encoded polypeptide sequences are shown in Table 10A.

TABLE 10A
NOV10 Sequence Analysis

NOV10a, CG185200-01 SEQ ID NO: 79 2050 bp
DNA Sequence ORF Start: ATG at 66 ORF Stop: TAA at 918
AGCACCATTTAAAGCCACTGGGAAATTTGTTGTCTAGTGGTTGTGGGTCAATAAAGGAGGGCAGAATG
GATGATTTCATCTCCATTAGCCTGCTGTCTCTGGCTATGTTGGTGGGATGTTACGTGGCCGGAATCAT
TCCCTTGGCTGTTAATTTCTCAGAGGAACGACTGAAGCTGGTGACTGTTTTGCGTGCTGGCCTTCTCT
GTGGAACTGCTCTGGCAGTCATCGTGCCTGAAGGAGTACATGCCCTTTATGAAGATATTCTTGAGGGA
AAACACCACCAAGCAAGTGAAACACATAATGTGATTGCATCAGACAAAGCAGCAGAAAAATCAGTTGT
CCATGAACATGAGCACAGCCACGACCACACACAGCTGCATGCCTATATTGGTGTTTCCCTCGTTCTGG
GCTTCGTTTTCATGTTGCTGGTGGACCAGATTGGTAACTCCCATGTGCATTCTACTGACGCTGATGGT
GTTGCTTTGGGAGCAGCAGCATCTACTTCACAGACCAGTGTCCAGTTAATTGTGTTTGTGGCAATCAT
GCTACATAAGGCACCAGCTGCTTTTGGACTGGTTTCCTTCTTGATGCATGCTCGCTTAGAGCGGAATC
GAATCAGAAAGCACTTGCTGGTCTTTGCATTGGCAGCACCAGTTACGTCCATGCTGACATACTTAGGA
CTGAGTAAGAGCAGTAAAGAAGCCCTTTCAGAGGTGAACGCCACGGGAGTGGCCATGCTTTTCTCTGC
CGGGACATTTCTTTATGTTGCCACAGTACATGTCCTCCCTGAGGTGGGCGGAATAGCGCACAGCCACA
AGCCCGATGCCACGGGAGGGAGAGGCCTCAGCCGCCTGGAAGTGGCAGCCCTGGTTCTCGGTTGCCTC
ATCCCTCTCATCCTGTCAGTAGCACACCAGCATTAAATGTTCAAGGTCCAGCCTTGGTCCAGGGCCGT
TTGCCATCCAGTGAGAACACCCGGCACGTGACAGCTACTCACTTCCTCAGTCTCTTGTCTCACCTTGC
GCATCTCTACATGTATTCCTAGAGTCCAGAGGGGAGGTGAGGTTAAAACCTGAGTAATGGAAAAGCTT
TTAGAGTAGAAACACATTTACGTTGCAGTTAGCTATAGACATCCCATTGTGTTATCTTTTAAAAGGCC
CTTGACATTTTGCGTTTTAATATTTCTCTTAACCCTATTCTCAGGGAAGATGGAATTTAGTTTTAAGG
AAAAGAGGAGAACTTCATACTCACAATGAAATAGTGATTATGAAAATACAGTGTTCTGTAATTAAGCT
ATGTCTCTTTCTTCTTAGTTTAGAGGCTCTGCTACTTTATCCATTGATTTTTAACATGGTTCCCACCA
TGTAAGACTGGTGCTTTAGCATCTATGCCACATGCGTTGATGGAAGGTCATACCACCCACTCACTTAG
ATGCTAAAGGTGATTCTAGTTAATCTGGGATTAGGGTCAGGAAAATGATACCAACACACATTGAAAGC
TCTCTTTATACTCAAAAGAGATATCCATTGAAAAGGGATGTCTAGAGGGATTTAAACAGCTCCTTTGG
CACGTGCCTCTCTCAATCCAGCCTGCCATTCCATCAAATGGAGCAGGAGAGCTCGCACGAGCTTCTAA
AGAGGTGACTGGTATTTTGTAGCATTCCTTGTCAAGTTCTCCTTTGCAGAATACCTGTCTCCACATTC
CTAGAGAGGAGCCAAGTTCTAGTAGTTTCAGTTCTAGGCTTTCCTTCAAGAACAGTCAGATCACAAAG
TGTCTTTGGAAATTAAGGGATATTAAATTTTAAGTGATTTTTGGATGGTTATTGATATCTTTCTAGTA
GCTTTTTTTAAAAGACTACCAAAATGTATGGTTGTCCTTTTTTTTTGTTTTTTTTTTTTTTAATTATT
TCTCTTACCACATCAGCAATCCCTCTAGGGACCTAAATACTACGTCAGCTTTGGCGACACTGTGTCTT
CTCACATAACCACCTGTAGCAAGATGGATCATAAATGAGAAGTGTTTCCCTATTGATTTAAAGCTTAT
TGGAATCATG
NOV10a, CG185200-01
Protein Sequence SEQ ID NO: 80 284 aa MW at 29900.4 kD
MDDFISISLLSLANLVGCYVAGIIPLAVNFSEERLKLVTVLGAGLLCGTALAVIVPEGVHALYEDILE
CKHHQASETHNVIASDKAAEKSVVHEHEHSHDHTQLHAYIGVSLVLGFVFMLLVDQIGNSHVHSTDAD
GVALGAAASTSQTSVQLIVFVAIMLHKAPAAFGLVSFLMHAGLERNRIRKHLLVFALAAPVTSMVTYL
GLSKSSKEALSEVNATGVANLFSAGTFLYVATVHVLPEVGGICHSHKPDATGGRGLSRLEVAALVLGC
LIPLILSVGHQH
NOV10b, CG185200-02 SEQ ID NO:81 1120 bp
DNA Sequence ORF Start: ATG at 94 ORF Stop: TAA at 1015
GGAACCACCACACCTGTTTAAAGAACCTAAGCACCATTTAAAGCCACTGGAAATTTGTTGTCTAGTGG
TTGTGGGTGAATAAAGGAGGGCAGAATGGATGATTTCATCTCCATTAGCCTGCTGTCTCTGGCTATGT
TGGTGGGATGTTACGTCGCCGGAATCATTCCCTTGGCTGTTAATTTCTCAGAGGAACGACTGAAGCTG
GTGACTGTTTTGGGTGCTGCCCTTCTCTGTGGAACTGCTCTGGCAGTCATCGTGCCTGAAGGAGTACA
TGCCCTTTATGAAGATATTCTTGAGGGAAAACACCACCAAGCAAGTGAAACACATAATGTGATTGCAT
CAGACAAAGCAGCAGAAAAATCAGTTGTCCATGAACATGAGCACAGCCACGACCACACACAGCTGCAT
GCCTATATTGGTGTTTCCCTCGTTCTGGGCTTCGTTTTCATGTTGCTGGTGGACCAGATTGGTAACTC
CCATGTGCATTCTACTGACGATCCAGAAGCAGCAAGGTCTAGCAATTCCAAAATCACCACCACGCTGG
GTCTGGTTGTCCATGCTGCAGCTGATGGTGTTGCTTTGGGAGCAGCAGCATCTACTTCACAGACCAGT
GTCCAGTTAATTGTGTTTGTGGCAATCATGCTACATAAGGCACCAGCTGCTTTTGGACTGGTTTCCTT
CTTGATGCATGCTGGCTTAGAGCGGAATCGAATCAGAAAGCACTTGCTGGTCTTTGCATTGGCAGCAC
CAGTTATGTCCATGGTGACATACTTAGGACTGAGTAAGAGCAGTAAAGAAGCCCTTTCAGAGGTGAAC
GCCACGGGAGTGGCCATGCTTTTCTCTGCCGGGACATTTCTTTATGTTGCCACAGTACATGTCCTCCC
TGAGGTGGGCGGAATAGGGCACAGCCACAAGCCCGATGCCGCGGGAGGGAGAGGCCTCAGCCGCCTGG
AAGTGGCAGCCCTGGTTCTGGGTTGCCTCATCCCTCTCATCCTGTCAGTACGACACCAGCATTAAATG
TTCAAGGTCCAGCCTTGGTCCAUGGCCGTTTGCCATCCAGTGAGAACAGCCGGCACGTGACAGCTACT
CACTTCCTCAGTCTCTTGTCTCACCTAAGGCG
NOV10b, CG185200-02
Protein Sequence SEQ ID NO: 82 307 aa MW at 32221.0 kD
MDDFISISLLSLAMLVGCYVAGIIPLAVNFSEERLKLVTVLGAGLLCGTALAVIVPEGVHALYEDILE
GKHHQASETHNVIASDKAAEKSVVHEHEHSHDHTQLHAYIGVSLVLGFVFMLLVDQIGNSHVHSTDDP
EAARSSNSKITTTLGLVVHAAADGVALGAAASTSQTSVQLIVFVAIMLHKAPAAFGLVSFLMHAGLER
NRIRKHLLVFALAAPVMSMVTYLGLSKSSKEALSEVNATGVAMLFSAGTFLYVATVHVLPEVGGIGHS
HKPDAAGGRGLSRLEVAALVLGCLIPLILSVGHQH

A ClustalW comparison of the above protein sequences yields the following sequence alignment shown in Table 10B.

TABLE 10B
Comparison of the NOV10 protein sequences.

| | |
|---|---|
| NOV10a | MDDFISISLLSLAMLVGCYVAGIIPLAVNFSEERLKLVTVLGAGLLCGTALAVIVPEGVH |
| NOV10b | MDDFISISLLSLAMLVGCYVAGIIPLAVNFSEERLKLVTVLGAGLLCGTALAVIVPEGVH |
| NOV10a | ALYEDILEGKHHQASETHNVIASDKAAEKSVVHEHEHSHDHTQLHAYIGVSLVLGFVFML |
| NOV10b | ALYEDILEGKHHQASETHNVIASDKAAEKSVVHEHEHSHDHTQLHAYIGVSLVLGFVFML |
| NOV10a | LVDQIGNSHVHSTD-----------------------ADGVALGAAASTSQTSVQLIVFV |
| NOV10b | LVDQIGNSHVHSTDDPEAARSSNSKITTTLGLVVHAAADGVALGAAASTSQTSVQLIVFV |
| NOV10a | AIMLHKAPAAFGLVSFLMHAGLERNRIRKHLLVFALAAPVTSMVTYLGLSKSSKEALSEV |
| NOV10b | AIMLNKAPAAFGLVSFLMHAGLERNRIRKHLLVFALAAPVMSMVTYLGLSKSSKEALSEV |
| NOV10a | NATGVAMLFSAGTFLYVATVHVLPEVGGIGHSHKPDATGGRGLSRLEVAALVLGCLIPLI |
| NOV10b | NATGVANLFSAGTFLYVATVHVLPEVGGIGHSHKPDATGGRGLSRLEVAALVLGCLIPLI |
| NOV10a | LSVGHQH |
| NOV10b | LSVGHQH |
| NOV10a | (SEQ ID NO: 80) |
| NOV10b | (SEQ ID NO: 82) |

Further analysis of the NOV10a protein yielded the following properties shown in Table 10C.

TABLE 10C
Protein Sequence Properties NOV10a

| SignalP | Cleavage site between residues 62 and 63 | | | | |
|---|---|---|---|---|---|
| analysis: | | | | | |
| PSORT II | PSG: a new signal peptide prediction method | | | | |
| analysis: | N-region: length 3; pos. chg 0; neg. chg 2 | | | | |
| | H-region: length 28; peak value 0.00 | | | | |
| | PSG score: −4.40 | | | | |
| | GvH: von Heijne's method for signal seq. recognition | | | | |
| | GvH score (threshold: −2.1): −1.40 | | | | |
| | possible cleavage site: between 52 and 53 | | | | |
| | >>> Seems to have no N-terminal signal peptide | | | | |
| | ALOM: Klein et al's method for TM region allocation | | | | |
| | Init position for calculation: 1 | | | | |
| | Tentative number of TMS(s) for the threshold 0.5: 7 | | | | |
| | INTEGRAL | Likelihood = | −6.48 | Transmembrane | 12-28 |
| | INTEGRAL | Likelihood = | −5.68 | Transmembrane | 38-54 |
| | INTEGRAL | Likelihood = | −8.49 | Transmembrane | 106-122 |
| | INTEGRAL | Likelihood = | −1.97 | Transmembrane | 153-169 |
| | INTEGRAL | Likelihood = | −3.13 | Transmembrane | 188-204 |
| | INTEGRAL | Likelihood = | −1.01 | Transmembrane | 221-237 |
| | INTEGRAL | Likelihood = | −8.81 | Transmembrane | 265-281 |
| | PERIPHERAL | Likelihood = | 9.18 (at 135) | | |

| ALOM score: −8.81 (number of TMSs: 7) | | | |
|---|---|---|---|
| MTOP: Prediction of membrane topology (Hartmann et al.) | | | |
| Center position for calculation: 19 | | | |
| Charge difference: 1.0 C(0.0)-N(−1.0) | | | |
| C > N: C-terminal side will be inside | | | |
| >>> membrane topology: type 3b | | | |
| MITDISC: discrimination of mitochondrial targeting seq | | | |
| R content: | 0 | Hyd Moment (75): | 8.48 |
| Hyd Moment (95): | 7.98 | G content: | 0 |
| D/E content: | 2 | S/T content: | 0 |

Score: −6.50
Gavel: prediction of cleavage sites for mitochondrial preseq
cleavage site motif not found
NUCDISC: discrimination of nuclear localization signals
pat4: none
pat7: none
bipartite: none
content of basic residues: 5.3%
NLS Score: −0.47
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 94.1
COIL: Lupas's algorithm to detect coiled-coil regions
total: 0 residues
Final Results (k = 9/23):
55.6%: endoplasmic reticulum
11.1%: Golgi
11.1%: vacuolar
11.1%: vesicles of secretory system
11.1%: mitochondrial
>> prediction for CG185200-01 is end (k = 9)

A search of the NOV10a protein against the Geneseq database, a proprietary database that contains sequences published in patents and patent publication, yielded several homologous proteins shown in Table 10D.

TABLE 10D
Geneseq Results for NOV10a

| | | NOV10a | Identities/ | |
|---|---|---|---|---|
| | | Residues/ | Similarities for | |
| Geneseq | Protein/Organism/Length [Patent | Match | the Matched | Expect |
| Identifier | #, Date] | Residues | Region | Value |
| AAB93646 | Human protein sequence SEQ ID | 1 . . . 284 | 284/307 (92%) | e−154 |
| | NO: 13148 - Homo sapiens, 307 aa. | 1 . . . 307 | 284/307 (92%) | |
| | [EP1074617-A2, 07 FEB. 2001] | | | |
| ABU57061 | Human PRO polypeptide #131 - | 1 . . . 284 | 283/307 (92%) | e−153 |
| | Homo sapiens, 307 aa. | 1 . . . 307 | 283/307 (92%) | |
| | [US2003027280-A1, 06 FEB. 2003] | | | |
| ABU56066 | Human secreted/transmembrane | 1 . . . 284 | 283/307 (92%) | e−153 |
| | protein, PRO1377 - Homo sapiens, | 1 . . . 307 | 283/307 (92%) | |
| | 307 aa. [US2003022298-A1, 30 | | | |
| | JAN. 2003] | | | |
| ABU10640 | Human secreted/transmembrane | 1 . . . 284 | 283/307 (92%) | e−153 |
| | protein #131 - Homo sapiens, 307 aa. | 1 . . . 307 | 283/307 (92%) | |
| | [US2002127584-A1, 12 SEP. 2002] | | | |
| AAB66116 | Protein of the invention #28 - | 1 . . . 284 | 283/307 (92%) | e−153 |
| | Unidentified, 307 aa. | 1 . . . 307 | 283/307 (92%) | |
| | [WO200078961-A1, 28 DEC. 2000] | | | |

In a BLAST search of public sequence databases, the NOV10a protein was found to have homology to the proteins shown in the BLASTP data in Table 10E.

TABLE 10E
Public BLASTP Results for NOV10a

| | | NOV10a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9NUM3 | Hypothetical protein FLJ11274 - | 1 . . . 284 | 284/307 (92%) | e−154 |
| | Homo sapiens (Human), 307 aa. | 1 . . . 307 | 284/307 (92%) | |
| AAH47682 | Hypothetical protein - Homo | 1 . . . 284 | 283/307 (92%) | e−153 |
| | sapiens (Human), 307 aa. | 1 . . . 307 | 283/307 (92%) | |
| Q8BFU1 | CDNA FLJ11274 FIS - Mus | 1 . . . 284 | 266/308 (86%) | e−143 |
| | musculus (Mouse), 308 aa. | 1 . . . 308 | 275/308 (88%) | |
| Q95JP5 | Hypothetical 25.0 kDa protein - | 130 . . . 284 | 149/155 (96%) | 2e−76 |
| | Macaca fascicularis (Crab eating | 82 . . . 235 | 152/155 (97%) | |
| | macaque) (Cynomolgus monkey), | | | |
| | 235 aa. | | | |
| AAH44279 | Hypothetical protein - Xenopus | 1 . . . 281 | 154/308 (50%) | 7e−69 |
| | laevis (African clawed frog), 303 aa. | 1 . . . 299 | 197/308 (63%) | |

PFam analysis predicts that the NOV10a protein contains the domains shown in the Table 10F.

TABLE 10F
Domain Analysis of NOV10a

| | | Identities/ | |
|---|---|---|---|
| | NOV10a | Similarities | |
| Pfam | Match | for the | Expect |
| Domain | Region | Matched Region | Value |
| Zip | 4 . . . 279 | 78/407 (19%) | 2.1e−34 |
| | | 214/407 (53%) | |

### Example 11

The NOV11 clone was analyzed, and the nucleotide and encoded polypeptide sequences are shown in Table 11A.

TABLE 11A
NOV11 Sequence Analysis

NOV11a, CG50513-01 SEQ ID NO: 83 1598 bp
DNA Sequence ORF Start: at 1 ORF Stop: TGA at 1354
AAACAGCCACTTGTTTCATCCCACCTGGGCATTAGGTTGACTTCAAAGATGCCTCAGTTACTGCAAAA
CATTAATGGGATCATCGAGGCCTTCAGGCGCTATGCAAGGACGGAGGGCAACTGCACAGCGCTCACCC
GAGGGGAGCTGAAAACACTCTTGGAGCAAGAGTTTGCCGATGTGATTGTGAAACCCCACGATCCAGCA
ACTGTGGATGAGGTCCTGCGTCTGCTGGATGAAGACCACACAGGGACTGTGGAATTCAAGGAATTCCT
GGTCTTAGTGTTTAAAGTTGCCCAGGCCTGTTTCAAGACACTGAGCGAGAGTGCTGAGGGAGCCTGCG
GCTCTCAAGAGTCTGGAAGCCTCCACTCTGGGGCCTCGCAGGAGCTGGGCGAAGGACAGAGAAGTGGC
ACTGAAGTGGGAAGGGCGGGGAAAGGGCAGCATTATGAGGGGAGCAGCCACAGACAGAGCCAGCAGGG
TTCCAGAGGGCAGAACAGGCCTGGGGTTCAGACCCAGGGTCAGGCCACTGGCTCTGCGTGGGTCAGCA
GCTATGACAGGCAAGCTGAGTCCCAGAGCCAGGAAAGAATAAGCCCGCAGATACAACTCTCTGGGCAG
ACAGAGCAGACCCAGAAGCTGGAGAAGGCAAGAGGGACTCAGACAACAGAGATGAGGCCAGAGAGACA
GCCACAGACCAGGGAACAGGACAGAGCCCACCAGACAGGTGAGACTGTGACTGGATCTGGAACTCAGA
CCCAGGCACGTGCCACCCAGACTGTGGAGCAGGACAGCAGCCACCAGACAGGAAGCACCAGCACCCAG
ACACAGGAGTCCACCAATGGCCAGAACAGAGGGACTGAGATCCACGGTCAAGGCAGGAGCCAGACCAG
CCAGGCTGTGACAGGAGGACACACTCAGATACAGGCAGGGTCACACACCGAGACTGTGGAGCAGGACA
GAAGCCAAACTGTAAGCCACGGAGGGGCTAGAGAACAGGCACAGACCCAGACCCAGCCACGCAGTGGT
CAAAGATGGATGCAAGTGAGCAACCCTGAGGCAGGAGAGACAGTACCGGGAGCACAGCCCCAGACTGG
GGCAAGCACTGAGTCAGGAAGGCAGGACTGGAGCAGCACTCACCCAAGGCGCTGTGTGACAGAAGGGC
AGGGAGACAGACACCCCACAGTGGTTGGTGAGGAATGGGTTGATGACCACTCAAGGGAGACAGTGATC
CTCAGGCTGGACCAGGGCAACTTGCATACCAGTGTTTCCTCAGCACAGGGCCAGGATGCAGCCCAGTC
AGAAGAGAAGCGAGGCATCACAGCTAGAGAGCTGTATTCCTACTTGAGAAGCACCAAGCCATGACTTC
CCCGACTCCAATGTCCAGTACTGCAAGAAGACAGCTGGAGAGAGTTTGCCTTGTCCTGCATGGCCAAT
CCAGTCCGTGCATCCCTGGACATCAGCTCTTCATTATGCAGCTTCCCTTTTAGGTCTTTCTCAATGAG
ATAATTTCTGCAAGGAGCTTTCTATCCTGAACTCTTCTTTCTTACCTGCTTTGCGGTCCAGACCCTCT
CAGGAGCAGGAAGACTCAGAACAAGTCACCCCTT
NOV11a, CG50513-01
Protein Sequence SEQ ID NO: 84 451 aa MW at 48908.6 kD
KQPLVSSHLGIRLTSKMPQLLQNINGIIEAFRRYARTEGNCTALTRGELKRLLEQEFADVIVKPHDPA
TVDEVLRLLDEDHTGTVEFKEFLVLVFKVAQACFKTLSESAEGACGSQESGSLHSGASQELGEGQRSG
TEVGRAGKGQHYEGSSHRQSQQGSRGQNRPGVQTQGQATGSAWVSSYDRQAESQSQERISPQIQLSGQ
TEQTQKAGEGKRNQTTEMRPERQPQTREQDRAHQTGETVTGSGTQTQAGATQTVEQDSSHQTGSTSTQ
TQESTNGQNRGTEIHGQGRSQTSQAVTGGHTQIQAGSHTETVEQDRSQTVSHGGAREQGQTQTQPGSG
QRWMQVSNPEAGETVPGGQAQTGASTESGRQEWSSTHPRRCVTEGQGDRQPTVVGEEWVDDHSRETVI
LRLDQCNLHTSVSSAQGQDAAQSEEKRGITARELYSYLRSTKP
NOV11b, 273654175 SEQ ID NO: 85 151 bp
DNA Sequence ORF Start: at 2 ORF Stop: at End of Sequence
ACCGGATCCTTACTGCAAAACATTAATGGGATCATCGAGGCCTTCAGGCGCTATGCAAGGACGGAGG
GCAACTGCACAGCGCTACCCGAGGGGAGCTGAAAAGACTCTTGGAGCAAGAGTTTGCCGATGTGATT
GTGAAACTCGAGGGC
NOV11b, 273654175
Protein Sequence SEQ ID NO: 86 50 aa MW at 5608.3 kD
TGSLLQNINGIIEAFRRYARTEGNCTALTRCELKRLLEQEFADVIVKLEG
NOV11c, CG50513-02 SEQ ID NO: 87 1039 bp
DNA Sequence ORF Start: at 1 ORF Stop: end of sequence
GTCAATGACAGCTTGTGTGATATGGTCCACCGTCCTCCAGCCATGAGCCAGGCCTGTAACACAGAGCC
CTGTCCCCCCAGGTGGCATGTGGGCTCTTGGGGGCCCTGCTCAGCTACCTGTGGAGTTGGAATTCAGA
CCCGAGATGTGTACTGCCTGCACCCAGGGGAGACCCCTGCCCCTCCTGAGGAGTGCCGAGATGAAAAG
CCCCATGCTTTACAAGCATGCAATCAGTTTGACTGCCCTCCTGGCTGGCACATTGAAGAATGGCAGCA
GTGTTCCAGGACTTGTGGCGGGGGAACTCAGAACAGAAGAGTCACCTGTCGGCAGCTGCTAACGGATG
GCAGCTTTTTGAATCTCTCAGATGAATTGTGCCAAGGACCCAAGGCATCGTCTCACAAGTCCTGTGCC
AGGACAGACTGTCCTCCACATTTAGCTGTGGGAGACTGGTCGAAGTGTTCTGTCAGTTGTGGTGTTCC
AATCCAGAGAAGAAAGCAGGTGTGTCAAAGGCTGGCAGCCAAAGGTCGGCGCATCCCCCTCAGTGAGA
TGATGTGCAGGGATCTACCAGGGTTCCCTCTTGTAAGATCTTGCCAGATGCCTGAGTGCAGTAAAATC
AAATCAGAGATGAAGACAAAACTTGGTGAGCAGGGTCCGCAGATCCTCAGTGTCCAGAGAGTCTACAT
TCAGACAAGGGAAGAGAAGCGTATTAACCTGACCATTGGTAGCAGAGCCTATTTGCTGCCCAACACAT
CCGTGATTATTAAGTGCCCAGTGCGACGATTCCAGAAATCTCTGATCCAGTGGGAGAAGGATGGCCGT
TGCCTGCAGAACTCCAAACGGCTTGGCATCACCAAGTCAGGCTCACTAAAAATCCACGGTCTTGCTGC
CCCCGACATCGGCGTGTACCGGTGCATTGCAGGCTCTGCACAGGAAACAGGTGTGCTCAAGCTCATTG
GTACTGACAACCGGCTCATTGCACGCCCAACCCTCANGGAGCCTATCAGGGAATATCCTCGGATGGAC
CACAACGAAGCCAATAGTT
NOV11c, CG50513-02
Protein Sequence SEQ ID NO: 88 346 aa MW at 38248.6 kD
VNDSLCDMVHRPPAMSQACNTEPCPPRWHVGSWGPCSATCCVGIQTRDVYCLHPGETPAPPEECRDEK
PHALQACNQFDCPPGWHIEEWQQCSRTCGGGTQNRRVTCRQLLTDGSFLNLSDELCQGPKASSHKSCA
RTDCPPHLAVGDWSKCSVSCGVGIQRRKQVCQRLAAKGRRIPLSEMMCRDLPCFPLVRSCQMPECSKI
KSEMKTKLGEQGPQILSVQRWIQTREEKRINLTIGSRAYLLPNTSVIIKCPVRRFQKSLIQWEKDGR
CLQNSKRLGITKSGSLKIHGLAAPDIGVYRCIAGSAQETGVLKLIGTDNRLIARPTLXEPMREYPGMD
HNEANS
NOV11d, CG50513-03 SEQ ID NO: 89 6303 bp
DNA Sequence ORF Start: ATG at 425 ORF Stop: TAA at 4268
TATAATTATTAATAGAGACCTTTCAAAGGACAAATTCTGTGAAATAAAGTGGTTTTCTGAAGAGCCTA
CTAATAGGACAGTGTGTTAATATCACTAATAAGAGAGTAATGATTATAAAAAGGAATAAATTTATTGA
AATTGCAAGATACTTTTCTCCTTTGATTAATATACTGCTAGTTTAGTTTTCTACATTTTCAAATAGAA
CTGGGGAATTTGTGTCGTAGATATTCTTGACAACTAAAGAGATGGTGGCTGAATTTTTGGGAATGGTT
GATAACACTTGATATTTTTAGTTTCCAATTTGGAAGAGCTCTGTCTCTTGGGATGTCAAATATTATAT
TCGTCAATTAATGAATGTCTTAATTTATTATAGAAATGATATTCTCACAATGATTTCATTTGTAGTGA
TGGATTTAAAGAGATAATGCCCTATCACCACTTCCAACCTCTTCCTCGCTGGGAACATAATCCTTGGA
CTGCATGTTCCGTGTCCTCTGGAGGAGGGATTCAGAGACGGAGCTTTGTGTGTGTAGAGGAATCCATG
CATGGAGAGATATTGCAGGTGGAAGAATGGAAGTGCATGTACGCACCCAAACCCAAGGTTATGCAAAC
TTGTAATCTGTTTGATTGCCCCAAGTGGATTGCCATGGAGTGGTCTCAGTGCACAGTGACTTGTGGCC
GAGGGTTACGGTACCGGGTTGTTCTGTGTATTAACCACCGCGGAGAGCATGTTGGGGCCTGCAATCCA
CAACTGAAGTTACACATCAAAGAAGAATGTGTCATTCCCATCCCGTGTTATAAACCAAAAGAAAAAAG
TCCAGTGGAAGCAAAATTGCCTTGGCTGAAACAAGCACAAGAACTAGAAGAGACCAGAATAGCAACAG
AAGAACCAACGTTCATTCCAGAACCCTGGTCAGCCTGCAGTACCACGTGTGGGCCGGGTGTGCAGGTC
CGTGAGGTGAAGTGCCGTGTGCTCCTCACATTCACGCAGACTGAGACTGAGCTGCCCGAGGAAGAGTG
TGAACGCCCCAAGCTGCCCACCGAACGGCCCTGCCTCCTGGAAGCATGTGATGAGAGCCCGGCCTCCC
GAGAGCTAGACATCCCTCTCCCTGAGGACAGTGAGACGACTTACGACTGGGAGTACGCTGGGTTCACC
CCTTGCACAGCAACATGCGTGGGAGGCCATCAAGAACCCATAGCAGTGTGCTTACATATCCAGACCCA
GCAGACAGTCAATGACAGCTTGTGTCATATGGTCCACCGTCCTCCAGCCATGAGCCAGGCCTGTAACA
CACAGCCCTGTCCCCCCAGGTGGCATGTGGGCTCTTGGGGGCCCTCCTCAGCTACCTGTGGAGTTGGA
ATTCAGACCCGAGATGTGTACTGCCTGCACCCAGGGGAGACCCCTGCCCCTCCTGAGGAGTGCCGAGA
TGAAAAGCCCCATGCTTTACAAGCATGCAATCAGTTTGACTGCCCTCCTGGCTGGCACATTGAAGAAT
GGCAGCACTGTTCCAGGACTTGTGGCGGGGGAACTCAGAACAGAAGAGTCACCTGTCGGCAGCTGCTA
ACGGATGGCAGCTTTTTGAATCTCTCAGATGAATTGTGCCAAGGACCCAAGGCATCGTCTCACAAGTC
CTGTGCCAGGACAGACTGTCCTCCACATTTAGCTGTGGGAGACTGGTCGAAGTGTTCTGTCAGTTGTG
GTGTTGGAATCCAGAGAAGAAAGCAGGTGTGTCAAAGGCTGGCAGCCAAAGGTCGGCGCATCCCCCTC
AGTGAGATGATGTGCAGGGATCTACCAGGGTTCCCTCTTGTAAGATCTTGCCAGATGCCTGAGTGCAG
TAAAATCAAATCAGAGATGAAGACAAAACTTGGTGAGCAGGGTCCGCAGATCCTCAGTGTCCAGAGAG
TCTACATTCAGACAAGGGAAGAGAAGCCTATTAACCTGACCATTGGTAGCAGAGCCTATTTGCTGCCC
AACACATCCGTGATTATTAAGTGCCCCGTGCGACGATTCCAGAAATCTCTGATCCAGTGGGAGAAGGA
TGGCCGTTGCCTGCAGAACTCCAAACGGCTTGGCATCACCAAGTCAGGCTCACTAAAAATCCACGGTC
TTGCTGCCCCCGACATCGGCGTGTACCGGTGCATTGCAGGCTCTGCACAGGAAACAGTTGTGCTCAAG
CTCATTGGTACTGACAACCGGCTCATCGCACGCCCAGCCCTCAGGGAGCCTATGAGGGAATATCCTGG
GATGGACCACAGCGAAGCCAATAGTTTGGGAGTCACATGGCACAAAATGAGGCAAATGTGGAATAACA
AAAATGACCTTTATCTGCATGATGACCACATTAGTAACCAGCCTTTCTTGAGAGCTCTGTTAGGCCAC
TGCAGCAATTCTGCAGGAAGCACCAACTCCTGGGAGTTGAAGAATAAGCAGTTTGAAGCAGCAGTTAA
ACAAGGAGCATATACCATGGATACACCCCAGTTTGATGAGCTGATAAGAAACATGAGTCAGCTCATGG
AAACCGGAGAGGTCAGCGATGATCTTGCGTCCCAGCTGATATATCAGCTGGTGGCCGAATTAGCCAAG
GCACAGCCAACACACATGCAGTGGCGGGGCATCCAGGAAGAGACACCTCCTGCTGCTCAGCTCAGAGG
GGAAACAGGGAGTGTGTCCCAAAGCTCGCATGCAAAAAACTCAGGCAAGCTGACATTCAAGCCGAAAG
GACCTGTTCTCATGAGGCAAAGCCAACCTCCCTCAATTTCATTTAATAAAACAATAAATTCCAGGATT
GGAAATACAGTATACATTACAAAAAGGACAGAGGTCATCAATATACTGTGTGACCTTATTACCCCCAG
TGAGGCCACATATACATGGACCAAGGATGGAACCTTGTTACAGCCCTCAGTAAAAATAATTTTGGATG
GAACTGGGAAGATACAGATACAGAATCCTACAAGGAAAGAACAACGCATATATGAATGTTCTGTAGCT
AATCATCTTGGTTCAGATGTGGAAAGTTCTTCTGTGCTGTATGCAGAGGCACCTGTCATCTTGTCTGT
TGAAAGAAATATCACCAAACCAGAGCACAACCATCTGTCTGTTGTGGTTGGAGGCATCGTGGAGGCAG
CCCTTGGAGCAAACGTGACAATCCGATGTCCTGTAAAAGGTGTCCCTCAGCCTAATATAACTTGGTTG
AAGAGAGGAGGATCTCTGAGTGGCAATGTTTCCTTGCTTTTCAATGGATCCCTGTTGTTGCAGAATGT
TTCCCTTGAAAATGAAGGAACCTACGTCTGCATAGCCACCAATGCTCTTGGAAAGGCAGTGGCAACAT
CTGTACTCCACTTGCTGGAACGAAGATGGCCAGAGAGTAGAATCGTATTTCTGCAAGGACATAAAAAG
TACATTCTCCAGGCAACCAACACTAGAACCAACAGCAATGACCCAACAGGAGAACCCCCGCCTCAAGA
GCCTTTTTGGGAGCCTGGTAACTGGTCACATTGTTCTGCCACCTGTGGTCATTTGGGAGCCCGCATTC
AGAGACCCCAGTGTGTGATGGCCAATGGGCAGGAAGTGAGTGAGGCCCTGTGTGATCACCTCCAGAAG
CCACTGGCTGGGTTTGAGCCCTGTAACATCCGGGACTGCCCAGCGAGGTGGTTCACAAGTGTGTGGTC
ACAGTGCTCTGTGTCTTGCGGTGAAGGATACCACAGTCGGCAGGTGACGTGCAAGCGGACAAAAGCCA
ATGGAACTGTGCAGGTGGTGTCTCCAAGAGCATGTGCCCCTAAAGACCGCCCTCTGGGAACAAAACCA
TGTTTTGGTCATCCATGTGTTCAGTGGGAACCAGGGAACCGGTGTCCTGGACGTTGCATGGGCCGTGC
TGTGAGGATGCAGCAGCGTCACACAGCTTGTCAACACAACAGCTCTGACTCCAACTGTGATGACAGAA
AGAGACCCACCTTAAGAAGGAACTGCACATCAGGGGCCTGTGATGTGTGTTGGCACACAGGCCCTTGG
AAGCCCTGTACAGCACCCTGTGGCAGGGGTTTCCAGTCTCGGAAAGTCGACTGTATCCACACAAGGAG
TTGCAAACCTGTGGCCAAGAGACACTGTGTACAGAAAAAGAAACCAATTTCCTGGCGGCACTGTCTTG
GGCCCTCCTGTGATAGAGACTGCACAGACACAACTCACTACTGTATGTTTGTAAAACATCTTAATTTC
TGTTCTCTAGACCGCTACAAACAAAGGTGCTGCCAGTCATGTCAAGAGGGATAAACCTTTGGAGGGGT
CATGATGCTGCTGTGAAGATAAAAGTAGAATATAAAAGCTCTTTTCCCCATGTCGCTGATTCAAAAAC
ATGTATTTCTTAAAAGACTAGATTCTATGGATCAAACAGAGGTTGATGCAAAAACACCACTGTTAAGG
TGTAAAGTGAAATTTTCCAATGGTAGTTTTATATTCCAATTTTTTAAAATGATGTATTCAAGGATGAA
CAAAATACTATAGCATGCATCCCACTGCACTTGGGACCTCATCATGTCAGTTCAATCGAGAAATCACC
AAGATTATGAGTGCATCCTCACGTGCTGCCTCTTTCCTGTGATATGTAGACTAGCACAGAGTGGTACA
TCCTAAAAACTTGCGAAACACAGCAACCCATGACTTCCTCTTCTCTCAAGTTGCAGGTTTTCAACAGT
TTTATAAGGTATTTGCATTTTAGAAGCTCTGGCCAGTAGTTGTTAAGATGTTGGCATTAATGGCATTT
TCATAGATCCTTGGTTTAGTCTGTGAAAAAGAAACCATCTCTCTGGATAGGCTGTCACACTGACTGAC
CTAAGGGTTCATGGAAGCATGGCATCTTGTCCTTGCTTTTAGAACACCCATGGAAGAAAACACAGAGT
AGATATTGCTGTCATTTATACAACTACAGAAATTTATCTATGACCTAATGAGGCATCTCGGAAGTCAA
AGAAGAGGGAAAGTTAACCTTTTCTACTGATTTCGTAGTATATTCAGAGCTTTCTTTTAAGAGCTGTG
AATGAAACTTTTTCTAAGCACTATTCTATTGCACACAAACAGAAAACCAAAGCCTTATTAGACCTAAT
TTATGCATAAAGTAGTATTCCTGAGAACTTTATTTTGGAAAATTTATAAGAAAGTAATCCAAATAAGA
AACACGATAGTTGAAAATAATTTTTATAGTAAATAATTGTTTTGGGCTGATTTTTCAGTAAATCCAAA
GTGACTTAGGTTAGAAGTTACACTAAGGACCAGGGGTTGGAATCAGAATTTAGTTTAAGATTTGAGGA
AAAGGGTAAGGGTTAGTTTCAGTTTTAGGATTAGAGCTAGAATTGGGTTAGGTGAGAAAQAAAGTTAA
GGTTAAGGCTAGAGTTGTCTTTAAGGGTTAGGGTTAGGACCAGGTTAGGTCAGGGTTGGATTGGGTTT
AGATTGGGGCCAGTGCTGGTGTTAGTGATAGTGTCAGGATGGAGGTTAGGTTTGGAGTAAGCGTTGTT
GCTGAAGTGAGTTCAGGCTAGCATTAAATTCTAAGTTCTGAAGCTGATTTGGTTATGGGGTCTTTCCC
CTGTATACTACCAGTTGTGTCTTTAGATGGCACACAAGTCCAAATAAGTGGTCATACTTCTTTATTCA
GGGTCTCAGCTGCCTGTACACCTGCTGCCTACATCTTCTTGGCAACAAAGTTACCTGCCACAGGCTCT
GCTGAGCCTAGTTCCTGGTCAGTAATAACTGAACAGTGCATTTTGGCTTTGGATGTGTCTGTGGACAA
GCTTGCTGAGTTTCTCTACCATATTCTGAGCACACGGTCTCTTTTGTTCTAATTTCAGCTTCACTGAC
ACTGGGTTGAGCACTACTGTATGTGGAGGGTTTGGTGATTGGGAATGGATGGGCGACAGTGAGGAGGA
CACACCAGCCCATTAGTTGTTAATCATCAATCACATCTGATTGTTGAAGGTTATTAAATTAAAAGAAA
GATCATTTGTAACATACTCTTTGTATATATTTATTATATGAAAGGTGCAATATTTTATTTTGTACAGT
ATGTAATAAAGACATGGGACATATATTTTTCTTATTAACAAAATTTCATATTAAATTGCTTCACTTTG
TATTTAAAGTTAAAAGTTACTATTTTTCATTTGCTATTGTACTTTCATTGTTGTCATTCAATTGACAT
TCCTGTGTACTGTATTTTACTACTGTTTTTATAACATGAGAGTTAATGTTTCTGTTTCATGATCCTTA
TGTAATTCAGAAATAAATTTACTTTGATTATTCAGTGGCATCCTTAT
NOV11d, CG50513-03
Protein Sequence SEQ ID NO: 90 1281 aa MW at 142825.9 kD
MPYDHFQPLPRWEHNPWTACSVSCGGGIQRRSFVCVEESMHGEILQVEEWKCMYAPKPKVMQTCNLFD
CPKWIANEWSQCTVTCGRGLRYRVVLCINHRGEHVGGCNPQLKLHIKEECVIPIPCYKPKEKSPVEAK
LPWLKQAQELEETRIATEEPTFIPEPWSACSTTCGPGVQVREVKCRVLLTFTQTETELPEEECEGPKL
PTERPCLLEACDESPASRELDIPLPEDSETTYDWEYAGFTPCTATCVGGHQEAIAVCLHIQTQQTVND
SLCDMVHRPPAMSQACNTEPCPPRWHVGSWGPCSATCGVGIQTRDVYCLHPGETPAPPEECRDEKPHA
LQACNQFDCPPGWHIEEWQQCSRTCGGGTQNRRVTCRQLLTDGSFLNLSDELCQGPKASSHKSCARTD
CPPHLAVGDWSKCSVSCCVGIQRRKQVCQRLAAKGRRIPLSEMMCRDLPGFPLVRSCQMPECSKIKSE
MKTKLGEQGPQILSVQRVYIQTREEKRINLTIGSRAYLLPNTSVIIKCPVRRFQKSLIQWEKDGRCLQ
NSKRLGITKSGSLKIHGLAAPDIGVYRCIAGSAQETVVLKLIGTDNRLIARPALREPMREYPGMDHSE
ANSLGVTWHKMRQMWNNKNDLYLDDDHISNQPFLRALLGHCSNSAGSTNSWELKNKQFEAAVKQGAYS
MDTAQFDELIRNMSQLMETGEVSDDLASQLIYQLVAELAKAQPTHMQWRGIQEETPPAAQLRGETGSV
SQSSHAKNSGKLTFKPKGPVLMRQSQPPSISFNKTINSRIGNTVYITKRTEVINILCDLITPSEATYT
WTKDGTLLQPSVKIILDGTGKIQIQNPTRKEQGIYECSVANHLGSDVESSSVLYAEAPVILSVERNIT
KPEHNHLSVVVGQIVEAALGANVTIRCPVKGVPQPNITWLKRGGSLSGNVSLLFNGSLLLQNVSLENE
GTYVCIATNALGKAVATSVLHLLERRWPESRIVFLQGHKKYILQATNTRTNSNDPTGEPPPQEPFWEP
GNWSHCSATCGHLGARIQRPQCVMANGQEVSEALCDHLQKPLAGFEPCNIRDCPARWFTSVWSQCSVS
CGEGYHSRQVTCKRTKANGTVQVVSPRACAPKDRPLURKPCFGHPCVQWEPGNRCPGRCNGRAVRMQQ
RHTACQHNSSDSNCDDRKRPTLRRNCTSGACDVCWHTGPWKPCTAACGRGFQSRKVDCIHTRSCKPVA
KRHCVQKKKPISWRHCLGPSCDRDCTDTTHYCMFVKHLNLCSLDRYKQRCCQSCQEG
NOV11e, CG50513-04 SEQ ID NO: 91 7260 bp
DNA Sequence ORF Start: ATG at 136 ORF Stop: TAA at 5209
CGCACGAGGTGTTGACGGGCGGCTTCTGCCAACTTCTCCCCAGCGCGCGCCGAGCCCGCGCGGCCCCG
GGGCTGCACGTCCCAGATACTTCTGCGGCGCAAGGCTACAACTGAGACCCGGAGGAGACTAGACCCCA
TGGCTTCCTGGACGAGCCCCTGGTGGGTGCTGATAGGGATCGTCTTCATGCACTCTCCCCTCCCGCAG
ACCACAGCTGAGAAATCTCCTGGAGCCTATTTCCTTCCCGAGTTTGCACTTTCTCCTCAGGGAAGTTT
TCTGUAAGACACAACAGGGGAGCAGTTCCTCACTTATCGCTATGATGACCAGACCTCAAGAAACACTC
GTTCAGATGAAGACAAAGATCGCAACTGGGATGCTTGGGGCGACTGGAGTGACTGCTCCCGGACCTGT
GGGGGAGGAGCATCATATTCTCTGCGCAGATGTTTGACTGGAAGGAATTGTGAAGGGCACAACATTCG
GTACAAGACATGCAGCAATCATGACTGCCCTCCAGATGCAGAAGATTTCAGAGCCCAGCAGTGCTCAG
CCTACAATGATGTCCAGTATCAGGGGCATTACTATGAATGGCTTCCACGATATAATCATCCTGCTGCC
CCGTGTGCACTCAAGTGTCATGCACAAGGACAAAACTTCGTGGTUGAGCTGGCACCTAAGGTACTGGA
TGGAACTCGTTGCAACACGGACTCCTTGGACATGTGTATCAGTGGCATCTGTCAGGCAGTGGGCTGCG
ATCGGCAACTGGGAAGCAATGCCAAGGAGGACAACTGTGGAGTCTGTGCCGGCGATGGCTCCACCTGC
AGGCTTGTACGGGGACAATCAAAGTCACACGTTTCTCCTGAAAAAAGAGAAGAAAATGTAATTGCTGT
TCCTTTGGGAAGTCGAAGTGTGAGAATTACAGTGAAAGGACCTGCCCACCTCTTTATTGAATCAAAAA
CACTTCAAGGAAGCAAAGGAGAACACAGCTTTAACAGCCCCGCCGTCTTTGTCGTAGAAAACACAACA
GTGGAATTTCACAGGGGCTCCGACAGGCAAACTTTTAAGATTCCAGGACCTCTGATGGCTGATTTCAT
CTTCAAGACCAGGTACACTGCAGCCAAAGACAGCGTGGTTCAGTTCTTCTTTTACCAGCCCATCAGTC
ATCAGTGGACACAAACTGACTTCTTTCCCTGCACTGTGACGTGTGGAGGAGGTTATCAGCTCAATTCT
GCTGAATGTGTGGATATCCGCTTGAAGAGGGTAGTTCCTGACCATTATTGTCACTACTACCCTGAAAA
TGTAAAACCAAAACCAAAACTGAAGGAATGCACCATGGATCCCTGCCCATCAAGTGATGGATTTAAAG
AGATAATGCCCTATGACCACTTCCAACCTCTTCCTCGCTGGGAACATAATCCTTGGACTGCATGTTCC
GTGTCCTGTGGAGGAGGGATTCAGAGACGGAGCTTTGTGTGTGTAGAGGAATCCATGCATGGAGAGAT
ATTGCAGGTGGAAGAATGGAAGTGCATGTACGCACCCAAACCCAAGGTTATGCAAACTTGTAATCTGT
TTGATTGCCCCAAGTGGATTGCCATGGAGTGGTCTCAGTGCACAGTGACTTGTGGCCGAGGGTTACGG
TACCGGGTTGTTCTGTGTATTAACCACCGCGGAGAGCATGTTGGGGGCTGCAATCCACAACTGAAGTT
ACACATCAAAGAAGAATGTGTCATTCCCATCCCGTGTTATAAACCAAAAGAAAAAAGTCCAGTGGAAG
CAAAATTGCCTTGGCTGAAACAAGCACAAGAACTAGAAGACACCAGAATAGCAACAGAACAACCAACG
TTCATTCCAGAACCCTGGTCAGCCTGCAGTACCACGTGTGGGCCGGGTGTGCAGGTCCGTGAGGTCAA
GTGCCGTGTGCTCCTCACATTCACGCAGACTGAGACTGAGCTGCCCGAGGAAGAGTGTGAAGGCCCCA
AGCTGCCCACCGAACGGCCCTGCCTCCTGGAAGCATGTCATGAGAGCCCGGCCTCCCGAGAGCTAGAC
ATCCCTCTCCCTGAGGACAGTGAGACGACTTACGACTGGGAGTACGCTGGGTTCACCCCTTCCACAGC
AACATCCGTGGGACGCCATCAAGAAGCCATAGCAGTGTGCTTACATATCCAGACCCAGCAGACAGTCA
ATGACAGCTTGTCTGATATGGTCCACCGTCCTCCAGCCATGAGCCAGGCCTGTAACACAGAGCCCTGT
CCCCCCAGGTGGCATGTGGGCTCTTGGGGGCCCTGCTCAGCTACCTGTGGAGTTGGAATTCAGACCCG
AGATGTGTACTGCCTGCACCCAGGGGAGACCCCTGCCCCTCCTGAGGAGTGCCGAGATGAAAAGCCCC
ATGCTTTACAAGCATGCAATCAGTTTGACTGCCCTCCTGGCTGGCACATTGAAGAATGGCAGCAGTGT
TCCAGAACTTGTGGCGGGGGAACTCAGAACAGAAGAGTCACCTGTCGGCAGCTGCTAACGGATGGCAG
CTTTTTCAATCTCTCAGATGAATTGTGCCAAGGACCCAAGGCATCGTCTCACAAGTCCTGTGCCAGGA
CAGACTGTCCTCCACATTTAGCTGTGGGAGACTGGTCGAAGTGTTCTGTCAGTTGTGGTGTTGGAATC
CAGAGAAGAAAGCAGGTGTGTCAAAGGCTGGCAGCCAAAGGTCGGCGCATCCCCCTCAGTGAGATGAT
GTGCAGGGATCTACCAGGGTTCCCTCTTGTAAGATCTTGCCAGATCCCTGAGTGCAGTAAAATCAAAT
CAGAGATGAAGACAAAACTTGGTGAGCAGGGTCCGCAGATCCTCAGTGTCCAGAGAGTCTACATTCAG
ACAAGGGAAGAGAAGCGTATTAACCTGACCATTGGTAGCAGAGCCTATTTGCTGCCCAACACATCCGT
GATTATTAAGTGCCCCGTGCGACGATTCCAGAAATCTCTGATCCAGTGGGAGAAGGATGGCCGTTGCC
TGCAGAACTCCAAACGGCTTGGCATCACCAAGTCAGGCTCACTAAAAATCCACGGTCTTGCTGCCCCC
GACATCGGCGTGTACCGGTGCATTGCAGGCTCTGCACAGGAAACAGTTGTGCTCAAGCTCATTGGTAC
TGACAACCGGCTCATCGCACGCCCAGCCCTCAGGGAGCCTATGAGGGAATATCCTGGGATGGACCACA
GCGAAGCCAATAGTTTGGCAGTCACATGGCACAAAATGAGGCAAATGTGGAATAACAAAAATGACCTT
TATCTGGATGATGACCACATTAGTAACCAGCCTTTCTTGAGAGCTCTGTTAGGCCACTGCAGCAATTC
TGCAGGAAGCACCAACTCCTGGGAGTTGAAGAATAAGCAGTTTGAAGCAGCAGTTAAACAAGGAGCAT
ATAGCATGGATACAGCCCAGTTTGATGAGCTGATAAGAAACATGAGTCAGCTCATGGAAACCGGAGAG
GTCAGCGATGATCTTGCGTCCCAGCTGATATATCAGCTGGTGGCCGAATTAGCCAAGGCACAGCCAAC
ACACATGCAGTGGCGGGGCATCCAGGAAGAGACACCTCCTGCTGCTCAGCTCAGAGGGGAAACAGGGA
GTGTGTCCCAAAGCTCGCATGCAAAAAACTCAGGCAAGCTGACATTCAAGCCGAAAGGACCTGTTCTC
ATGAGGCAAAGCCAACCTCCCTCAATTTCATTTAATAAAACAATAAATTCCAGGATTGGAAATACAGT
ATACATTACAAAAAGGACAGAGGTCATCAATATACTGTGTGACCTTATTACCCCCAGTGAGGCCACAT
ATACATGGACCAAGGATGGAACCTTGTTACAGCCCTCAGTAAAAATAATTTTGGATGGAACTGGGAAG
ATACAGATACAGAATCCTACAAGGAAAGAACAAGGCATATATGAATGTTCTGTAGCTAATCATCTTGG
TTCAGATGTGGAAAGTTCTTCTGTGCTGTATGCAGAGCCACCTGTCATCTTGTCTGTTGAAAGAAATA
TCACCAAACCAGAGCACAACCATCTGTCTGTTGTGGTTGGAGGCATCGTGGAGGCAGCCCTTGGACCA
AACGTGACAATCCGATGTCCTGTAAAAGGTGTCCCTCAGCCTAATATAACTTGGTTGAAGAGAGGAGG
ATCTCTGAGTGGCAATGTTTCCTTGCTTTTCAATGGATCCCTGTTGTTGCAGAATGTTTCCCTTGAAA
ATGAAGGAACCTACGTCTGCATAGCCACCAATGCTCTTGGAAAGGCAGTGGCAACATCTGTATTCCAC
TTGCTGGAACGAAGATGGCCAGAGAGTAGAATCGTATTTCTGCAAGGACATAAAAAGTACATTCTCCA
GGCAACCAACACTAGAACCAACAGCAATGACCCAACAGGAGAACCCCCGCCTCAAGAGCCTTTTTGGC
AGCCTGGTAACTGGTCACATTGTTCTGCCACCTGTGGTCATTTGGGAGCCCGCATTCAGAGACCCCAG
TGTGTGATGGCCAATGGGCAGGAAGTGAGTGAGGCCCTGTGTGATCACCTCCAGAAGCCACTGGCTGG
GTTTGAGCCCTGTAACATCCGGGACTGCCCAGCGAGGTGGTTCACAAGTGTGTGGTCACAGTGCTCTG
TGTCTTGCGGTGAAGGATACCACAGTCOGCAGGTGACGTGCAAGCGGACAAAAGCCAATGGAACTGTG
CAGGTGGTGTCTCCAAGAGCATGTGCCCCTAAAGACCGGCCTCTGGGAAGAAAACCATGTTTTGGTCA
TCCATGTGTTCAGTGGGAACCAGGGAACCGGTGTCCTGGACGTTGCATGGGCCGTGCTGTGAGCATGC
AGCAGCGTCACACAGCTTGTCAACACAACAGCTCTGACTCCAACTGTGATGACAGAAAGAGACCCACC
TTAAGAAGGAACTGCACATCAGCCGCCTGTGATGTGTGTTGGCACACAGGCCCTTGGAAGCCCTGTAC
AGCAGCCTGTGGCAGGGGTTTCCAGTCTCGGAAAGTCGACTGTATCCACACAAGGAGTTGCAAACCTG
TGGCCAAGAGACACTGTGTACAGAAAAAGAAACCAATTTCCTGGCGGCACTGTCTTGGGCCCTCCTGT
GATAGAGACTGCACAGACACAACTCACTACTGTATGTTTGTAAAACATCTTAATTTGTGTTCTCTAGA
CCGCTACAAACAAAGGTGCTGCCAGTCATGTCAAGAGGGATAAACCTTTGGAGGGGTCATGATGCTGC
TGTGAAGATAAAAGTAGAATATAAAAGCTCTTTTCCCCATGTCGCTGATTCAAAAACATGTATTTCTT
AAAAGACTAGATTCTATGGATCAAACAGAGGTTGATGCAAAAACACCACTGTTAAGGTGTAAAGTGAA
ATTTTCCAATGGTAGTTTTATATTCCAATTTTTTAAAATGATGTATTCAAGGATGAACAAAATACTAT
AGCATGCATGCCACTGCACTTGGGACCTCATCATGTCAGTTGAATCGAGAAATCACCAAGATTATGAG
TGCATCCTCACGTGCTGCCTCTTTCCTGTGATATGTAGACTAGCACAGAGTGGTACATCCTAAAAACT
TGGGAAACACAGCAACCCATGACTTCCTCTTCTCTCAAGTTGCAGGTTTTCAACAGTTTTATAAGGTA
TTTGCATTTTAGAAGCTCTGGCCAGTAGTTGTTAAGATGTTGGCATTAATGCCATTTTCATAGATCCT
TGGTTTAGTCTGTGAAAAAGAAACCATCTCTCTGGATAGGCTGTCACACTGACTGACCTAAGGGTTCA
TGGAAGCATGGCATCTTGTCCTTGCTTTTAGAACACCCATGGAAGAAAACACAGAGTAGATATTGCTG
TCATTTATACAACTACAGAAATTTATCTATGACCTAATGAGGCATCTCGGAAGTCAAAGAAGAGGGAA
AGTTAACCTTTTCTACTGATTTCGTAGTATATTCAGAGCTTTCTTTTAAGAGCTGTGAATGAAACTTT
TTCTAAGCACTATTCTATTGCACACAAACAGAAAACCAAAGCCTTATTAGACCTAATTTATGCATAAA
GTACTATTCCTGAGAACTTTATTTTGGAAAATTTATAAGAAAGTAATCCAAATAAGAAACACGATAGT
TGAAAATAATTTTTATAGTAAATAATTGTTTTGGGCTGATTTTTCAGTAAATCCAAAGTGACTTAGGT
TAGAAGTTACACTAAGGACCAGGGGTTGGAATCAGAATTTAGTTTAAGATTTGAGGAAAAGGGTAAGG
GTTAGTTTCAGTTTTAGGATTAGAGCTAGAATTGGGTTAGGTGAGAAAGAAAGTTAAGGTTAAGGCTA
GAGTTGTCTTTAAGGGTTAGGGTTAGGACCAGGTTAGGTCAGGGTTGGATTGGGTTTAGATTGGGGCC
AGTGCTGGTGTTAGTGATAGTGTCAGGATGGAGGTTAGGTTTGGAGTAAGCGTTGTTGCTGAAGTGAG
TTCAGGCTAGCATTAAATTGTAAGTTCTGAAGCTGATTTCGTTATGGGGTCTTTCCCCTGTATACTAC
CAGTTGTGTCTTTAGATGGCACACAAGTCCAAATAAGTGGTCATACTTCTTTATTCAGGGTCTCAGCT
GCCTGTACACCTGCTGCCTACATCTTCTTGGCAACAAAGTTACCTGCCACAGGCTCTGCTGAGCCTAG
TTCCTGGTCAGTAATAACTGAACAGTGCATTTTGGCTTTGGATGTGTCTGTGGACAAGCTTGCTGAGT
TTCTCTACCATATTCTGAGCACACGGTCTCTTTTGTTCTAACTTCAGCTTCACTGACACTGGGTTGAG
CACTACTGTATGTGGAGGGTTTGGTGATTGGGAATGGATGGGGGACAGTGAGGAGGACACACCAGCCC
ATTAGTTGTTAATCATCAATCACATCTGATTGTTGAAGGTTATTAAATTAAAAGAAAGATCATTTGTA
ACATACTCTTTGTATATATTTATTATATGAAAGGTGCAATATTTTATTTTGTACAGTATGTAATAAAG
ACATGGGACATATATTTTTCTTATTAACAAAATTTCATATTAAATTGCTTCACTTTGTATTTAAAGTT
AAAAGTTACTATTTTTCATTTGCTATTGTACTTTCATTGTTGTCATTCAATTGACATTCCTGTGTACT
GTATTTTACTACTGTTTTTATAACATGAGAGTTAATGTTTCTGTTTCATGATCCTTATGTAATTCAGA
AATAAATTTACTTTGATTATTCAGTGGCATCCTTATAAAAAAAAAAAAAAAA
NOV11e, CG50513-04
Protein Sequence SEQ ID NO: 92 1691 aa MW at 188743.8 kD
NASWTSPWWVLIGMVFMHSPLPQTTAEKSPGAYFLPEFALSPQGSFLEDTTGEQFLTYRYDDQTSRNT
RSDEDKDGNWDAWGDWSDCSRTCGGGASYSLRRCLTGRNCEGQNIRYKTCSNHDCPPDAEDFRAQQCS
AYNDVQYQGHYYEWLPRYNDPAAPCALKCHAQGQNLVVELAPKVLDGTRCNTDSLDMCISUICQAVGC
DRQLGSNAKEDNCGVCACDGSTCRLVRGQSKSHVSPEKREENVIAVPLGSRSVRITVKGPAHLFIESK
TLQGSKGEHSFNSPGVFVVENTTVEFQRGSERQTFKIPGPLMADFIFKTRYTAAKDSVVQFFFYQPIS
HQWRQTDFFPCTVTCGGGYQLNSAECVDIRLKRVVPDHYCHYYPENVKPKPKLKECSMDPCPSSDGFK
EIMPYDHFQPLPRWEHNPWTACSVSCGGGIQRRSFVCVEESMHGEILQVEEWKCMYAPKPKVMQTCNL
FDCPKWIAMEWSQCTVTCGRGLRYRVVLCINHRGEHVGGCNPQLKLHIKEECVIPIPCYKPKEKSPVE
AKLPWLKQAQELEETRIATEEPTFIPEPWSACSTTCGPGVQVREVKCRVLLTFTQTETELPEEECEGP
KLPTERPCLLEACDESPASRELDIPLPEDSETTYDWEYAGFTPCTATCVGGHQEAIAVCLHIQTQQTV
NDSLCDMVHRPPANSQACNTEPCPPRWHVGSWGPCSATCGVGIQTRDVYCLHPGETPAPPEECRDEKP
HALQACNQFDCPPGWHIEEWQQCSRTCGGGTQNRRVTCRQLLTDGSFLNLSDELCQGPKASSHKSCAR
TDCPPHLAVGDWSKCSVSCGVGIQRRKQVCQRLAAKGRRIPLSEMMCRDLPGFPLVRSCQMPECSKIK
SEMKTKLGEQGPQILSVQRVYIQTREEKRINLTIGSRAYLLPNTSVIIKCPVRRFQKSLIQWEKDGRC
LQNSKRLGITKSGSLKIHGLAAPDIGVYRCIAGSAQETVVLKLIGTDNRLIARPALREPMREYPGMDH
SEANSLGVTWHKMRQMWNNKNDLYLDDDHISNQPFLRALLGHCSNSAGSTNSWELKNKQFEAAVKQGA
YSMDTAQFDELIRNMSQLMETGEVSDDLASQLIYQLVAELAKAQPTHMQWRGIQEETPPAAQLRGETG
SVSQSSHAKNSGKLTFKPKGPVLMRQSQPPSISFNKTINSRIGNTVYITKRTEVINILCDLITPSEAT
YTWTKDGTLLQPSVKIILDGTGKIQIQNPTRKEQGIYECSVANHLGSDVESSSVLYAEAPVILSVERN
ITKPEHNHLSVVVGGIVEAALGANVTIRCPVKGVPQPNITWLKRGGSLSGNVSLLFNGSLLLQNVSLE
NEGTYVCIATNALGKAVATSVFHLLERRWPESRIVFLQGHKKYILQATNTRTNSNDPTGEPPPQEPFW
EPGNWSHCSATCGHLGARIQRPQCVMANGQEVSEALCDHLQKPLAGFEPCNIRDCPARWFTSVWSQCS
VSCGEGYHSRQVTCKRTKANGTVQVVSPRACAPKDRPLGRKPCFGHPCVQWEPGNRCPGRCMGRAVRM
QQRHTACQHNSSDSNCDDRKRPTLRRNCTSGACDVCWHTGPWKPCTAACGRGFQSRKVDCIHTRSCKP
VAKRHCVQKKKPISWRHCLGPSCDRDCTDTTHYCMFVKHLNLCSLDRYKQRCCQSCQEG
NOV11f, CG50513-05 SEQ ID NO: 93 6294 bp
DNA Sequence ORF Start: ATG at 416 ORF Stop: TAA at 4259
TAATAGAGACCTTTCAAAGGACAAATTCTGTGAAATAAAGTGGTTTTCTGAAGAGCCTACTAATAGGA
CAGTGTGTTAATATCACTAATAAGAGAGTAATGATTATAAAAAGGAATAAATTTATTGAAATTGCAAC
ATACTTTTCTCCTTTGATTAATATACTGCTAGTTTACTTTTCTACATTTTCAAATAGAACTGGGGAAT
TTGTGTCGTAGATATTCTTGACAACTAAAGAGATGGTGGCTGAATTTTTGGGAATGGTTGATAACACT
TGATATTTTTAGTTTCCAATTTGGAAGAGCTCTGTCTCTTGGGATGTCAAATATTATATTCGTCAATT
AATCAATGTGTTAATTTATTATAGAAATGATATTCTCACAATGATTTCATTTGTAGTGATGGATTTAA
AGAGATAATGCCCTATGACCACTTCCAACCTCTTCCTCGCTGGGAACATAATCCTTGGACTGCATGTT
CCGTGTCCTGTGGAGGAGGGATTCAGAGACGGAGCTTTGTGTGTGTAGAGGAATCCATGCATCGAGAG
ATATTGCAGGTGGAAGAATGGAAGTGCATGTACGCACCCAAACCCAAGGTTATGCAAACTTGTAATCT
GTTTGATTGCCCCAAGTGGATTGCCATGGAGTGGTCTCAGTGCACAGTGACTTGTGUCCGAGGGTTAC
GGTACCGGGTTGTTCTGTGTATTAACCACCGCGGAGAGCATGTTGGGGGCTGCAATCCACAACTGAAG
TTACACATCAAAGAAGAATGTGTCATTCCCATCCCGTGTTATAAACCAAAAGAAAAAAGTCCAGTGGA
AGCAAAATTGCCTTGGCTGAAACAAGCACAAGAACTAGAAGAGACCAGAATAGCAACAGAAGAACCAA
CGTTCATTCCAGAACCCTGGTCAGCCTGCAGTACCACGTGTGGGCCGGGTGTGCAGGTCCGTGAGGTG
AAGTGCCGTGTGCTCCTCACATTCACGCAGACTGAGACTGAGCTGCCCGAGGAAGAGTGTGAAGGCCC
CAAGCTGCCCACCGAACGGCCCTGCCTCCTGGAAGCATGTGATGAGAGCCCGGCCTCCCGAGAGCTAG
ACATCCCTCTCCCTGAGGACAGTGAGACCACTTACGACTGGCAGTACGCTGGGTTCACCCCTTGCACA
GCAACATGCGTGGGAGGCCATCAAGAAGCCATAGCAGTGTGCTTACATATCCAGACCCAGCAGACAGT
CAATGACAGCTTGTGTGATATGGTCCACCGTCCTCCAGCCATGAGCCAGGCCTGTAACACAGAGCCCT
GTCCCCCCAGGTGGCATGTGGGCTCTTGGGGGCCCTGCTCACCTACCTGTGGAGTTGGAATTCAGACC
CGAGATGTGTACTGCCTGCACCCAGGGGAGACCCCTGCCCCTCCTGAGGAGTGCCGAGATGAAAAGCC
CCATGCTTTACAAGCATGCAATCAGTTTGACTGCCCTCCTGGCTGGCACATTGAAGAATGGCAGCAGT
GTTCCAGGACTTGTGGCGGGGGAACTCAGAACAGAACAGTCACCTGTCGGCAGCTGCTAACGGATGGC
AGCTTTTTGAATCTCTCAGATGAATTGTGCCAAGGACCCAAGGCATCGTCTCACAAGTCCTGTGCCAG
GACAGACTGTCCTCCACATTTAGCTGTGGGAGACTGGTCGAAGTGTTCTGTCAGTTGTGGTGTTGGAA
TCCAGAGAAGAAAGCAGGTGTGTCAAAGGCTGGCAGCCAAAGGTCGGCGCATCCCCCTCAGTGAGATG
ATGTGCAGGGATCTACCAGGGCTCCCTCTTGTAAGATCTTGCCAGATGCCTGAGTGCAGTAAAATCAA
ATCAGAGATGAAGACAAAACTTGGTGAGCAGGGTCCGCAGATCCTCAGTGTCCAGAGAGTCTACATTC
AGACAAGGGAAGAGAAGCGTATTAACCTGACCATTGGTAGCAGACCCTATTTGCTGCCCAACACATCC
GTGATTATTAAGTGCCCAGTGCGACGATTCCAGAAATCTCTGATCCAGTGGGAGAAGGATGGCCGTTG
CCTGCAGAACTCCAAACGGCTTGGCATCACCAAGTCAGGCTCACTAAAAATCCACGGTCTTGCTGCCC
CCGACATCGGCGTGTACCGGTGCATTGCAGGCTCTGCACAGGAAACAGTTGTGCTCAAGCTCATTGGT
ACTGACAACCGGCTCATCGCACGCCCAGCCCTCAGGGAGCCTATGAGGGAATATCCTGGGATGGACCA
CAGCGAAGCCAATAGTTTGGGAGTCACATGGCACAAAATGAGGCAAATGTGGAATAACAAAAATGACC
TTTATCTGGATGATGACCACATTAGTAACCAGCCTTTCTTGAGAGCTCTGTTAGGCCACTGCAGCAAT
TCTGCAGGAAGCACCAACTCCTGGGAGTTGAAGAATAAGCAGTTTGAAGCAGCAGTTAAACAAGGAGC
ATATAGCATGGATACAGCCCAGTTTGATGAGCTGATAAGAAACATGAGTCAGCTCATGGAAACCGGAG
AGGTCAGCGATGATCTTGCGTCCCAGCTGATATATCAGCTGGTGGCCGAATTAGCCAAGGCACAGCCA
ACACACATGCAGTGGCGGCGCATCCAGGAAGAGACACCTCCTGCTGCTCAGCTCAGAGGGGAAACAGG
GAGTGTGTCCCAAAGCTCGCATGCAAAAAACTCAGGCAAGCTGACATTCAAGCCGAAAGGACCTGTTC
TCATGAGGCAAAGCCAACCTCCCTCAATTTCATTTAATAAAACAATAAATTCCAGGATTGGAAATACA
GTATACATTACAAAAAGGACACAGGTCATCAATATACTGTGTGACCTTATTACCCCCAGTGAGGCCAC
ATATACATGGACCAAGGATGGAACCTTGTTACACCCCTCAGTAAAAATAATTTTGGATGGAACTGGGA
AGATACAGATACAGAATCCTACAAGGAAAGAACAAGGCATATATGAATGTTCTGTAGCTAATCATCTT
GGTTCAGATGTGGAAAGTTCTTCTGTGCTGTATGCAGAGGCACCTGTCATCTTGTCTGTTGAAAGAAA
TATCACCAAACCAGAGCACAACCATCTGTCTGTTGTGGTTGGAGGCATCGTGGAGGCAGCCCTTGGAG
CAAACCTGACAATCCGATGTCCTGTAAAAGGTGTCCCTCAGCCTAATATAACTTGGTTGAAGAGAGGA
GGATCTCTGAGTGGCAATGTTTCCTTGCTTTTCAATGGATCCCTGTTGTTGCAGAATGTTTCCCTTCA
AAATGAAGGAACCTACGTCTGCATAGCCACCAATGCTCTTGGAAAGGCAGTGGCAACATCTGTACTCC
ACTTGCTGGAACGAAGATGGCCAGAGAGTAGAATCGTATTTCTGCAAGGACATAAAAAGTACATTCTC
CAGGCAACCAACACTAGAACCAACAGCAATGACCCAACAGGAGAACCCCCGCCTCAAGAGCCTTTTTG
GGAGCCTGGTAACTGGTCACATTGTTCTCCCACCTGTGGTCATTTGGGAGCCCGCATTCAGAGACCCC
AGTGTGTGATCGCCAATGGGCAGGAAGTGAGTGAGGCCCTGTGTGATCACCTCCAGAAGCCACTGGCT
GGGTTTGAGCCCTGTAACATCCGGGACTGCCCAGCGAGGTGGTTCACAAGTGTGTGGTCACAGTGCTC
TGTGTCTTGCGGTGAAGGATACCACAGTCGGCAGGTGACGTGCAAGCGGACAAAAGCCAATGGAACTG
TGCAGGTGGTGTCTCCAAGAGCATGTGCCCCTAAAGACCGCCCTCTGGGAAGAAAACCATGTTTTGGT
CATCCATGTGTTCAGTGGGAACCAGGCAACCGGTGTCCTCGACGTTGCATGCGCCGTGCTGTGAGGAT
GCAGCAGCGTCACACAGCTTGTCAACACAACACCTCTGACTCCAACTGTGATGACAGAAAGAGACCCA
CCTTAAGAAGGAACTGCACATCAGGGGCCTGTGATGTGTGTTGGCACACAGGCCCTTGGAAGCCCTGT
ACAGCAGCCTGTGGCAGGGGTTTCCAGTCTCGGAAAGTCGACTGTATCCACACAAGGAGTTGCAAACC
TGTGGCCAAGAGACACTGTGTACAGAAAAAGAAACCAATTTCCTGGCGGCACTGTCTTGGGCCCTCCT
GTGATAGAGACTGCACAGACACAACTCACTACTGTATGTTTGTAAAACATCTTAATTTGTGTTCTCTA
GACCGCTACAAACAAAGGTGCTGCCAGTCATGTCAAGAGGGATAAACCTTTGGAGGGGTCATGATGCT
GCTGTCAAGATAAAAGTAGAATATAAAAGCTCTTTTCCCCATGTCGCTGATTCAAAAACATGTATTTC
TTAAAAGACTAGATTCTATGGATCAAACAGAGGTTGATGCAAAAACACCACTGTTAAGGTGTAAAGTG
AAATTTTCCAATGGTAGTTTTATATTCCAATTTTTTAAAATGATGTATTCAAGGATGAACAAAATACT
ATAGCATGCATGCCACTGCACTTGGGACCTCATCATGTCAGTTGAATCGAGAAATCACCAAGATTATG
AGTGCATCCTCACGTGCTGCCTCTTTCCTGTGATATGTAGACTAGCACAGAGTGGTACATCCTAAAAA
CTTGGGAAACACAGCAACCCATGACTTCCTCTTCTCTCAAGTTGCAGGTTTTCAACAGTTTTATAAGG
TATTTGCATTTTAGAAGCTCTGGCCAGTAGTTGTTAAGATGTTGGCATTAATGGCATTTTCATAGATC
CTTGGTTTAGTCTGTGAAAAAGAAACCATCTCTCTGGATAGGCTGTCACACTGACTGACCTAAGGGTT
CATGGAAGCATGGCATCTTGTCCTTGCTTTTAGAACACCCATGGAAGAAAACACAGAGTAGATATTGC
TGTCATTTATACAACTACAGAAATTTATCTATGACCTAATGACGCATCTCGGAAGTCAAAGAAGAGGG
AAAGTTAACCTTTTCTACTGATTTCGTAGTATATTCAGAGCTTTCTTTTAAGAGCTGTGAATGAAACT
TTTTCTAAGCACTATTCTATTGCACACAAACAGAAAACCAAAGCCTTATTAGACCTAATTTATGCATA
AAGTAGTATTCCTGAGAACTTTATTTTGGAAAATTTATAAGAAAGTAATCCAAATAAGAAACACGATA
GTTGAAAATAATTTTTATAGTAAATAATTGTTTTGGGCTGATTTTTCAGTAAATCCAAAGTGACTTAG
GTTAGAAGTTACACTAACGACCACGGGTTGGAATCAGAATTTAGTTTAAGATTTGAGGAAAAGGGTAA
GGGTTAGTTTCAGTTTTAGGATTAGAGCTAGAATTGGGTTAGGTGAGAAAGAAAGTTAAGGTTAAGGC
TAGAGTTGTCTTTAAGGGTTAGGCTTAGGACCAGGTTAGGTCAGGGTTGGATTGGGTTTAGATTGGCG
CCAGTGCTGGTGTTAGTGATAGTGTCAGGATGGAGGTTAGGTTTGGAGTAAGCGTTGTTGCTGAAGTG
AGTTCAGGCTAGCATTAAATTGTAAGTTCTGAAGCTGATTTGGTTATGGGGTCTTTCCCCTGTATACT
ACCAGTTGTGTCTTTAGATGGCACACAAGTCCAAATAAGTGGTCATACTTCTTTATTCAGGGTCTCAG
CTGCCTGTACACCTGCTGCCTACATCTTCTTGGCAACAAAGTTACCTGCCACAGGCTCTGCTGAGCCT
AGTTCCTGGTCAGTAATAACTGAACAGTGCATTTTGGCTTTGGATGTGTCTGTGGACAAGCTTGCTGA
GTTTCTCTACCATATTCTGAGCACACGGTCTCTTTTGTTCTAATTTCAGCTTCACTGACACTGGGTTG
AGCACTACTGTATGTGGAGGGTTTGGTGATTGGGAATGGATGGGGGACAGTGAGGAGGACACACCAGC
CCATTAGTTGTTAATCATCAATCACATCTGATTGTTGAAGGTTATTAAATTAAAAGAAAGATCATTTG
TAACATACTCTTTGTATATATTTATTATATGAAAGGTGCAATATTTTATTTTGTACAGTATGTAATAA
AGACATGGGACATATATTTTTCTTATTAACAAAATTTCATATTAAATTGCTTCACTTTGTATTTAAAG
TTAAAAGTTACTATTTTTCATTTGCTATTGTACTTTCATTGTTGTCATTCAATTGACATTCCTGTGTA
CTGTATTTTACTACTGTTTTTATAACATGAGAGTTAATGTTTCTGTTTCATGATCCTTATGTAATTCA
GAAATAAATTTACTTTGATTATTCAGTGGCATCCTTAT
NOV11f, CG50513-05
Protein Sequence SEQ ID NO: 94 1281 aa MW at 142791.9 kD
MPYDHFQPLPRWEHNPWTACSVSCGGGIQRRSFVCVEESMHGEILQVEEWKCMYAPKPKVMQTCNLFD
CPKWIAMEWSQCTVTCGRGLRYRVVLCINHRGEHVGGCNPQLKLHIKEECVIPIPCYKPKEKSPVEAK
LPWLKQAQELEETRIATEEPTFIPEPWSACSTTCGPGVQVREVKCRVLLTFTQTETELPEEECEGPKL
PTERPCLLEACDESPASRELDIPLPEDSETTYDWEYAGFTPCTATCVGGHQEAIAVCLHIQTQQTVND
SLCDMVHRPPANSQACNTEPCPPRWHVGSWGPCSATCGVGIQTRDVYCLHPCETPAPPEECRDEKPHA
LQACNQFDCPPGWHIEEWQQCSRTCGGGTQNRRVTCRQLLTDGSFLNLSDELCQGPKASSHKSCARTD
CPPHLAVGDWSKCSVSCGVGIQRRKQVCQRLAAKGRRIPLSEMMCRDLPGLPLVRSCQNPECSKIKSE
MKTKLGEQGPQILSVQRVYIQTREEKRINLTIGSRAYLLPNTSVIIKCPVRRFQKSLIQWEKDGRCLQ
NSKRLGITKSGSLKIHGLAAPDIGVYRCIAGSAQETVVLKLIGTDNRLIARPALREPMREYPGMDHSE
ANSLGVTWHKMRQMWNNKNDLYLDDDHISNQPFLRALLGHCSNSAGSTNSWELKNKQFEAAVKQGAYS
MDTAQFDELIRNMSQLMETGEVSDDLASQLIYQLVAELAKAQPTHMQWRGIQEETPPAAQLRGETGSV
SQSSHAKNSGKLTFKPKGPVLMRQSQPPSISFNKTINSRIGNTVYITKRTEVINILCDLITPSEATYT
WTKDGTLLQPSVKIILDGTGKIQIQNPTRKEQGIYECSVANHLGSDVESSSVLYAEAPVILSVERNIT
KPEHNHLSVVVGGIVEAALGANVTIRCPVKGVPQPNITWLKRGGSLSGNVSLLFNGSLLLQNVSLENE
GTYVCTATNALGKAVATSVLHLLERRWPESRIVFLQGHKKYILQATMTRTNSNDPTGEPPPQEPFWEP
GNWSHCSATCGHLGARIQRPQCVMANGQEVSEALCDHLQKPLAGFEPCNIRDCPARWFTSVWSQCSVS
CGEGYHSRQVTCKRTKANGTVQVVSPRACAPKDRPLGRKPCFGHPCVQWEPGNRCPGRCMGRAVRNQQ
RHTACQHNSSDSNCDDRKRPTLRRNCTSGACDVCWHTGPWKPCTAACGRGFQSRKVDCIHTRSCKPVA
KRHCVQKKKPISWRHCLGPSCDRDCTDTTHYCMFVKHLNLCSLDRYKQRCCQSCQEG
NOV11g, CG50513-06 SEQ ID NO: 95 2912 bp
DNA Sequence ORF Start: ATG at 98 ORF Stop: TAA at 2876
CAGCTTTAACAGCCCCGGCGTCTTTGTCGTAGAAAACACAACAGTGGAATTTTAGAGGGGCTCCGAGA
GGCAAACTTTTAAGATTCCAGGCCCTTTGATGGCTGATTTCATCTTCAAGACCAGGTACACTGCAGCC
AAAGACAGCGTGGTTCAGTTCTTCTTTTACCAGCCCATCAGTCATCAGTGGAGACAAACTGACTTCTT
TCCCTGCACTGTGACGTGTGGAGGAGGTTATCAGCTCAATTCTGCTGAATGTGTGGATATCCGCTTGA
AGAGGGTAGTTCCTGACCATTATTGTCACTACTACCCTGAAAATGTAAAACCAAAACCAAAACTGAAG
GAATGCAGCATGGATCCCTGCCCATCAAGTGATGGATTTAAAGAGATAATGCCCTATGACCACTTCCA
ACCTCTTCCTCGCTGGGAACATAATCCTTGGACTGCATGTTCCGTGTCCTGTGGAGGAGGGATTCAGA
GACGGAGCTTTGTGTGTGTAGAGGAATCCATGCATGGAGAGATATTGCAGGTGGAAGAATGGAAGTGC
ATGTACGCACCCAAACCCAAGGTTATGCAAACTTGTAATCTGTTTGATTGCCCCAAGTGGATTGCCAT
GGAGTGGTCTCAGTGCACAGTGACTTGTGGCCGAGGCTTACGCTACCGGGTTGTTCTGTOTATTAACC
ACCGCGGAGAGCATGTTGGGGCCTGCAATCCACAACTGAAGTTACACATCAAAGAAGAATGTGTCATT
CCCATCCCGTGTTATAAACCAAAAGAAAAAAGTCCAGTGGAAGCAAAATTGCCTTGGCTGAAACAAGC
ACAAGAACTAGAAGAGACCAGAATAGCAACAGAAGAACCAACGTTCATTCCAGAACCCTGGTCAGCCT
GCAGTACCACGTGTGGGCCGGGTGTGCAGGTCCGTGAGGTGAAGTGCCGTGTGCTCCTCACATTCACG
CAGACTGAGACTGAGCTGCCCGAGGAAGAGTGTCAAGGCCCCAAGCTGCCCACCGAACGGCCCTGCCT
CCTQGAAGCATGTGATGAGAGCCCGGCCTCCCGAGAGCTAGACATCCCTCTCCCTGAGGACAGTGAGA
CGACTTACGACTGGGAGTACGCTGGGTTCACCCCTTGCACAGCAACATGCGTGGGAGGCCATCAAGAA
GCCATAGCAGTGTGCTTACATATCCAGACCCAGCAGACAGTCAATGACAGCTTGTGTGATATGGTCCA
CCGTCCTCCAGCCATGAGCCAGGCCTGTAACACAGAGCCCTGTCCCCCCACGTGGCATGTGGGCTCTT
GGGGGCCCTGCTCAGCTACCTGTGGAGTTGGAATTCAGACCCGAGATGTGTACTGCCTGCACCCAGGG
GAGACCCCTGCCCCTCCTGAGGAGTGCCGAGATGAAAAGCCCCATGCTTTACAAGCATGCAATCAGTT
TGACTGCCCTCCTGGCTGGCACATTGAAGAATGGCAGCAGTGTTCCAGGACTTGTGGCGGGGGAACTC
AGAACAGAAGAGTCACCTGTCGGCAGCTCCTAACGGATGGCAGCTTTTTGAATCTCTCAGATGAATTG
TGCCAAGGACCCAAGGCATCGTCTCACAAGTCCTGTGCCAGGACAGACTGTCCTCCACATTTAGCTGT
GGGAGACTGGTCGAAGTGTTCTGTCAGTTGTGGTGTTGGAATCCAGAGAAGAAAGCAGGTGTGTCAAA
GGCTGGCAGCCAAAGGTCGGCGCATCCCCCTCAGTGAGATGATGTGCAGGGATCTACCAGGGTTCCCT
CTTGTAAGATCTTGCCAGATGCCTGAGTGCAGTAAAATCAAATCAGAGATGAAGACAAAACTTGGTGA
GCAGGGTCCGCAGATCCTCAGTGTCCAGAGAGTCTACATTCAGACAAGGGAAGAGAAGCGTATTAACC
TGACCATTGGTAGCAGAGCCTATTTGCTGCCCAACACATCCGTGATTATTAAGTGCCCAGTGCGACGA
TTCCAGAAATCTCTGATCCAGTGGGAGAAGGATGGCCGTTGCCTCCAGAACTCCAAACGGCTTGGCAT
CACCAAGTCAGGCTCACTAAAAATCCACGGTCTTGCTGCCCCCGACATCGGCGTGTACCGGTGCATTG
CAGGCTCTGCACAGGAAACAGTTGTGCTCAAGCTCATTGGTACTGACAACCGGCTCATCGCACGCCCA
GCCCTCAGGGAGCCTATGAGGGAATATCCTGGGATGGACCACAGCGAAGCCAATAGTTTGGGAGTCAC
ATGGCACAAAATGAGGCAAATGTGGAATAACAAAAATGACCTTTATCTGGATGATGACCACATTAGTA
ACCACCCTTTCTTGAGAGCTCTGTTAGGCCACTGCAGCAATTCTGCAGCAAGCACCAACTCCTGGGAG
TTGAAGAATAAGCAGTTTGAAGCAGCAGTTAAACAAGGAGCATATAGCATGGATACAGCCCAGTTTGA
TGAGCTGATAAGAAACATGAGTCAGCTCATGGAAACCGGAGAGGTCAGCGATGATCTTGCGTCCCAGC
TGATATATCAGCTGGTGGCCGAATTAGCCAAGGCACAGCCAACACACATGCAGTGGCGGGGCATCCAG
GAAGAGACACCTCCTGCTGCTCAGCTCAGAGGCGAAACAGGGAGTGTGTCCCAAAGCTCGCATGCAAA
AAACTCAGGCAAGCTGACATTCAAGCCGAAAGGACCTGTTCTCATGAGGCAAAGCCAACCTCCCTCAA
TTTCATTTAATAAAACAATAAATTCCAGGATTGGAAATACAGTATACATTACAAAAAGGACAGAGGTC
ATCAATATACTGTGTGACCTTATTACCCCCAGTGAGGCCACATATACATGGACCAAGGATGGAACCTT
GTTACAGCCCTCAGTAAAGTAAGTAAAATAAAAATGCAGTATTCATTTTTGCAAAA
NOV11g, CG50513-06
Protein Sequence SEQ ID NO: 96 926 aa MW at 104117.1 kD
MADFIFKTRYTAAKDSVVQFFFYQPISHQWRQTDFFPCTVTCGGGYQLMSAECVDIRLKRVVPDHYCH
YYPENVKPKPKLKECSMDPCPSSDGFKEEMPYDHFQPLPRWEHNPWTACSVSCGGGIQRRSFVCVEES
MHGEILQVEEWKCMYAPKPKVMQTCNLFDCPKWIAMEWSQCTVTCGRGLRYRVVLCINHRGEHVGCCN
PQLKLHIKEECVIPIPCYKPKEKSPVEAKLPWLKQAQELEETRIATEEPTFIPEPWSACSTTCGPGVQ
VREVKCRVLLTFTQTETELPEEECEGPKLPTERPCLLEACDESPASRELDIPLPEDSETTYDWEYAGF
TPCTATCVGCHQEAIAVCLHIQTQQTVNDSLCDMVHRPPAMSQACNTEPCPPRWHVGSWGPCSATCGV
GIQTRDVYCLHPGETPAPPEECRDEKPHALQACNQFDCPPGWHIEEWQQCSRTCGGGTQNRRVTCRQL
LTDGSFLNLSDELCQGPKASSHKSCARTDCPPHLAVGDWSKCSVSCGVGIQRRKQVCQRLAAKGRRIP
LSEMMCRDLPGFPLVRSCQMPECSKIKSEMKTKLGEQGPQILSVQRVYIQTREEKRINLTIGSRAYLL
PNTSVIIKCPVRRFQKSLIQWEKDGRCLQNSKRLCITKSGSLKIHGLAAPDIGVYRCIAGSAQETVVL
KLIGTDNRLIARPALREPMREYPGMDHSEANSLGVTWHKMRQMWNNKNDLYLDDDHISNQPFLRALLG
HCSNSAGSTNSWELKNKQFEAAVKQGAYSMDTAQFDELIRNMSQLMETGEVSDDLASQLIYQLVAELA
KAQPTHMQWRGIQEETPPAAQLRGETGSVSQSSHAKNSGKLTFKPKGPVLMRQSQPPSISFNKTINSR
IGNTVYITKRTEVINILCDLITPSEATYTWTKDGTLLQPSVK
NOV11h, CG50513-07 SEQ ID NO: 97 1377 bp
DNA Sequence ORF Start: at 1 ORF Stop: end of sequence
TGGGAACATAATCCTTGGACTGCATGTTCCGTGTCCTGTGGAGGAGGGATTCAGAGACGGAGCTTTGT
GTGTGTAGAGGAATCCATGCATGGAGAGATATTGCAGGTGGAAGAATGGAAGTGCATGTACGCACCCA
AACCCAAGGTTATGCAAACTTGTAATCTGTTTGATTGCCCCAACTGGATTGCCATGGAGTGGTCTCAG
TGCACAGTGACTTGTGGCCGAGGGTTACGGTACCGGGTTGTTCTGTGTATTAACCACCGCGGAGAGCA
TGTTGGGGGCTGCAATCCACAACTGAAGTTACACATCAAAGAAGAATGTGTCATTCCCATCCCGTGTT
ATAAACCAAAAGAAAAAAGTCCAGTGGAAGCAAAATTGCCTTGGCTGAAACAAGCACAAGAACTAGAA
GAGACCAGAATAGCAACAGAAGAACCAACGTTCATTCCAGAACCCTGGTCAGCCTGCAGTACCACGTG
TGGGCCGGGTGTGCAGGTCCGTGAGGTGAAGTGCCGTGTGCTCCTCACATTCACGCAGACCGAGACTG
AGCTGCCCGAGGAAGAGTGTGAAGGCCCCAAGCTGCCCACCGAACGCCCCTGCCTCCTGGAAGCATGT
GATGAGAGCCCGGCCTCCCGAGAGCTAGACATCCCTCTCCCTGAGGACAGTGAGACGACTTACGACTG
GGAGTACGCTGGGTTCACCCCTTGCACAGCAACATGCGTGGGAGGCCATCAAGAAGCCATAGCAGTGT
GCTTACATATCCAGACCCAGCAGACAGTCAATGACAGCTTGTGTGATATGGTCCACCGTCCTCCAGCC
ATGAGCCAGOCCTGTAACACAGAGCCCTGTCCCCCCAGGTGGCATGTGGGCTCTTGGGGGCCCTGCTC
AGCTACCTGTGGAGTTGGAATTCAGACCCGAGATGTGTACTGCCTGCACCCAGGGGAGACCCCTGCCC
CTCCTGAGGAGTGCCOAGATGAAAAQCCCCATGCTTTACAAGCATGCAATCAGTTTGACTGCCCTCCT
GGCTGGCACATTGAAGAATOGCAGCAGTGTTCCAGGACTTGTGGCGGGGGAACTCACAACAGAAGAGT
CACCTGTCGOCAGCTGCTAACGGATOGCAGCTTTTTGAATCTCTCAGATGAATTGTGCCAAGGACCCA
AGGCATCGTCTCACAAGTCCTGTGCCAGGACAGACTGTCCTCCACATTTAGCTGTGGGAGACTGGTCG
AAGTGTTCTOTCAGTTGTGGTGTTGGAATCCAGAGAAGAAAGCAGGTGTGTCAAAGGCTGGCAGCCAA
AGGTCGGCGCATCTCCCTCAGTGAGATGATGTGCAGGGATCTACCAGGGCTCCCTCTTGTAAGATCTT
GCCAGATGCCTGAGTGC
NOV11h, CG50513-07
Protein Sequence SEQ ID NO: 98 459 aa MW at 51217.0 kD
WEHNPWTACSVSCGGGIQRRSFVCVEESMNGEILQVEEWKCMYAPKPKVMQTCNLFDCPKWIAMEWSQ
CTVTCGRGLRYRVVLCINHRGEHVGGCNPQLKLHIKEECVIPIPCYKPKEKSPVEAKLPWLKQAQELE
ETRIATEEPTFIPEPWSACSTTCGPGVQVREVKCRVLLTFTQTETELPEEECEGPKLPTERPCLLEAC
DESPASRELDIPLPEDSETTYDWEYAGFTPCTATCVGGHQEAIAVCLHIQTQQTVNDSLCDMVHRPPA
MSQACNTEPCPPRWHVGSWGPCSATCGVGIQTRDVYCLHPGETPAPPEECRDEKPHALQACNQFDCPP
OWNIEEWQQCSRTCGGGTQNRRVTCRQLLTDGSFLNLSDELCQGPKASSHKSCARTDCPPHLAVGDWS
KCSVSCGVGIQRRKQVCQRLAAKGRRISLSEMMCRDLPGLPLVRSCQMPEC
NOV11i, 13376798 SNP
for CG50513-01 SEQ ID NO: 99 1598 bp SNP: position 58, T/A
DNA Sequence ORF Start: at 1 ORF Stop: TGA at 1354
AAACAGCCACTTGTTTCATCCCACCTGGGCATTAGGTTGACTTCAAAGATGCCTCAGATACTGCAAAAC
ATTAATOGGATCATCGAGGCCTTCAGGCGCTATGCAAGGACGGAGGGCAACTGCACAGCGCTCACCCGA
GGGGAGCTGAAAAGACTCTTGGAGCAAGAGTTTGCCGATGTGATTGTGAAACCCCACGATCCAGCAACT
GTGGATGAGGTCCTGCGTCTGCTGGATGAAGACCACACAGGGACTGTGGAATTCAAGGAATTCCTGGTC
TTAGTGTTTAAAGTTGCCCACGCCTGTTTCAAGACACTGAGCGAGAGTGCTGACGGAGCCTGCOGCTCT
CAAGAGTCTGGAAGCCTCCACTCTGGGGCCTCGCAGGAGCTGGGCGAAGGACAGAGAAGTGGCACTGAA
GTGGGAAGGGCGGGGAAAGGGCAGCATTATGAGGGGAGCAGCCACAGACAGAGCCAGCAGGGTTCCAGA
GGGCAGAACAGGCCTGGGGTTCAGACCCAGGGTCAGGCCACTGGCTCTGCGTGGGTCAGCAGCTATGAC
AGGCAAGCTGAGTCCCAGAGCCAGGAAAGAATAAGCCCGCAGATACAACTCTCTGGGCAGACAGAGCAG
ACCCAGAAAGCTGGAGAAGGCAAGAGGAATCACACAACAGAGATGAGGCCAGAGAGACAGCCACAGACC
AGGGAACAGGACAGAGCCCACCAGACAGGTGAGACTGTGACTGGATCTGGAACTCAGACCCAGGCAGGT
GCCACCCAGACTGTGGAGCAGGACAGCAGCCACCAGACAGGAAGCACCAGCACCCAGACACAGGAGTCC
ACCAATGGCCAGAACAGAGGGACTGAGATCCACGGTCAAGGCAGGAGCCAGACCAGCCAGGCTGTGACA
GGAGGACACACTCAGATACAGGCAGGGTCACACACCGAGACTGTGGAGCAGGACAGAAGCCAAACTGTA
AGCCACGGACGGGCTAGAGAACAGGGACAGACCCAGACGCAGCCAGGCAGTGGTCAAAGATGGATGCAA
GTGAGCAACCCTGAGGCAGGAGAGACAGTACCGGGAGGACAGGCCCAGACTGGGGCAAGCACTGAGTCA
GGAAGGCAGGAGTGGAGCAGCACTCACCCAAGGCGCTGTGTGACAGAAGGGCAGGGAGACAGACAGCCC
ACAGTGGTTGGTGAGGAATGGGTTGATGACCACTCAAGGGAGACAGTGATCCTCAGGCTGGACCAGGGC
AACTTGCATACCAGTGTTTCCTCAGCACAGGGCCAGGATGCAGCCCAGTCAGAAGAGAAGCGAGGCATC
ACAGCTAGAGAGCTGTATTCCTACTTGAGAAGCACCAAGCCATGACTTCCCCGACTCCAATGTCCAGTA
CTGGAAGAAGACAGCTGGAGAGAGTTTGGCTTGTCCTGCATGGCCAATCCAGTGGGTCCATCCCTGGAC
ATCAGCTCTTCATTATGCAGCTTCCCTTTTAGGTCTTTCTCAATGAGATAATTTCTGCAAGGAGCTTTC
TATCCTGAACTCTTCTTTCTTACCTGCTTTGCGGTGCAGACCCTCTCAGGAGCAGGAAGACTCAGAACA
AGTCACCCCTT
NOV11i, 13376798 SNP
for CG50513-01 SNP: Leu to Ile
Protein Sequence SEQ ID NO: 100 451 aa at position 20
KQPLVSSHLGIRLTSKMPQILQNINGIIEAFRRYARTEGNCTALTRGELKRLLEQEFADVIVKPHDPAT
VDEVLRLLDEDHTGTVEFKEFLVLVFKVAQACFKTLSESAEGACGSQESGSLHSGASQELGEGQRSGTE
VGRAGKGQHYEGSSHRQSQQGSRGQNRPGVQTQGQATGSAWVSSYDRQAESQSQERISPQIQLSGQTEQ
TQKAGEGKRNQTTEMRPERQPQTREQDRAHQTGETVTGSGTQTQAGATQTVEQDSSHQTGSTSTQTQES
TNOQNRGTEIHGQGRSQTSQAVTGGHTQIQAGSHTETVEQDRSQTVSHGGAREQGQTQTQPGSGQRWMQ
VSNPEAGETVPGGQAQTGASTESGRQEWSSTHPRRCVTEGQGDRQPTVVGEEWVDDHSRETVILRLDQG
NLHTSVSSAQGQDAAQSEEKRGITARELYSYLRSTKP
NOV11j, 13376799 SNP 1598 bp,
for CG50513-01 SEQ ID NO: 101 SNP: T/C at position 1516
DNA Sequence ORF Start: at 1 ORF Stop: TGA at 1354
AAACAGCCACTTGTTTCATCCCACCTGGGCATTAGGTTGACTTCAAAGATGCCTCAGTTACTGCAAAAC
ATTAATGGGATCATCGAGGCCTTCAGGCGCTATGCAAGGACGGAGGGCAACTGCACAGCGCTCACCCGA
GGGGAGCTGAAAAGACTCTTGGAGCAAGAGTTTGCCGATGTGATTGTGAAACCCCACGATCCAGCAACT
GTGGATGAGGTCCTGCGTCTGCTGGATGAAGACCACACACGGACTGTGGAATTCAAGGAATTCCTGGTC
TTAGTGTTTAAAGTTGCCCAGGCCTGTTTCAAGACACTGAGCGAGAGTGCTGAGGGAGCCTGCGGCTCT
CAAGAGTCTCGAAGCCTCCACTCTGGGGCCTCGCAGGAGCTGGGCGAAGGACAGAGAAGTGGCACTGAA
GTGGGAAGGGCGGGGAAAGGGCAGCATTATGAGGGGAGCAGCCACAGACAGAGCCAGCAGGGTTCCAGA
GGGCAGAACAGGCCTGGGGTTCAGACCCAGGGTCAGGCCACTGGCTCTGCGTGGGTCAGCAGCTATGAC
AGGCAAGCTGAGTCCCAGAGCCAGGAAAGAATAAGCCCGCAGATACAACTCTCTGGGCAGACAGAGCAG
ACCCAGAAAGCTGCAGAAGGCAAGAGGAATCAGACAACAGAGATGAGCCCACAGAGACAGCCACAGACC
AGGGAACAGGACAGAGCCCACCAGACAGGTGAGACTGTGACTGGATCTGGAACTCAGACCCAGGCAGGT
GCCACCCAGACTGTGGAGCAGGACAGCAGCCACCAGACAGGAAGCACCAGCACCCAGACACAGGAGTCC
ACCAATGGCCAGAACAGAGGGACTGAGATCCACGGTCAAGGCAGGAGCCAGACCAGCCAGGCTGTGACA
GGAGGACACACTCAGATACAGGCAGGGTCACACACCGAGACTGTGGAGCAGGACAGAAGCCAAACTGTA
AGCCACGGAGGGGCTAGAGAACAGGGACACACCCAGACGCAGCCAGGCAGTGGTCAAAGATGGATGCAA
GTGAGCAACCCTGAGGCAGGAGAGACAGTACCGGGAGGACAGGCCCAGACTGGGGCAAGCACTGAGTCA
GCAAGGCAGGAGTGGAGCAGCACTCACCCAAGCCGCTGTGTGACAGAAGGGCAGGGAGACAGACAGCCC
ACAGTGGTTGGTGAGGAATGGGTTGATGACCACTCAAGGGAGACAGTGATCCTCAGGCTGGACCAGGGC
AACTTGCATACCAGTGTTTCCTCAGCACAGGGCCAGGATGCAGCCCAGTCAGAACAGAAGCGAGGCATC
ACAGCTAGAGAGCTGTATTCCTACTTGACAAGCACCAAGCCATGACTTCCCCGACTCCAATGTCCAGTA
CTGGAAGAAGACAGCTGGAGAGAGTTTGGCTTGTCCTGCATGGCCAATCCAGTGGGTGCATCCCTGGAC
ATCACCTCTTCATTATGCAGCTTCCCTTTTAGGTCTTTCTCAATGAGATAATTTCTGCAAGGAGCTCTC
TATCCTGAACTCTTCTTTCTTACCTGCTTTGCGGTGCAGACCCTCTCAGGAGCAGGAAGACTCAGAACA
AGTCACCCCTT
NOV11j, 13376799 SNP
for CG50513-01
Protein Sequence SEQ ID NO: 102 451 aa SNP: not in coding region
KQPLVSSHLGIRLTSKMPQLLQNINGIIEAFRRYARTEGNCTALTRGELKRLLEQEFADVIVKPHDPAT
VDEVLRLLDEDHTGTVEFKEFLVLVFKVAQACFKTLSESAEGACGSQESGSLHSGASQELGEGQRSGTE
VGRAGKGQHYEGSSHRQSQQGSRGQNRPGVQTQGQATGSAWVSSYDRQAESQSQERISPQIQLSGQTEQ
TQKAGEGKRNQTTEMRPERQPQTREQDRAHQTGETVTGSGTQTQAGATQTVEQDSSHQTGSTSTQTQES
TNGQNRGTEIHGQGRSQTSQAVTGGHTQIQAGSHTETVEQDRSQTVSHGGAREQGQTQTQPGSGQRWMQ
VSNPEAGETVPGGQAQTGASTESGRQEWSSTHPRRCVTEGQGDRQPTVVGEEWVDDHSRETVILRLDQG
NLHTSVSSAQGQDAAQSEEKRGITARELYSYLRSTKP

A ClustalW comparison of the above protein sequences yields the following sequence alignment shown in Table 11B.

TABLE 11B
Comparison of the NOV11 protein sequences.

| | |
|---|---|
| NOV11a | ------------------------------------------------------------ |
| NOV11b | ------------------------------------------------------------ |
| NOV11c | ------------------------------------------------------------ |
| NOV11d | ------------------------------------------------------------ |
| NOV11e | MASWTSPWWVLIGMVFMHSPLPQTTAEKSPGAYFLPEFALSPQGSFLEDTTGEQFLTYRY |
| NOV11f | ------------------------------------------------------------ |
| NOV11g | ------------------------------------------------------------ |
| NOV11h | ------------------------------------------------------------ |
| NOV11a | ------------------------------------------------------------ |
| NOV11b | ------------------------------------------------------------ |
| NOV11c | ------------------------------------------------------------ |
| NOV11d | ------------------------------------------------------------ |
| NOV11e | DDQTSRNTRSDEDKDGNWDAWGDWSDCSRTCGGGASYSLRRCLTGRNCEGQNIRYKTCSN |
| NOV11f | ------------------------------------------------------------ |
| NOV11g | ------------------------------------------------------------ |
| NOV11h | ------------------------------------------------------------ |
| NOV11a | ------------------------------------------------------------ |
| NOV11b | ------------------------------------------------------------ |
| NOV11c | ------------------------------------------------------------ |
| NOV11d | ------------------------------------------------------------ |
| NOV11e | HDCPPDAEDFRAQQCSAYNDVQYQGHYYEWLPRYNDPAAPCALKCHAQGQNLVVELAPKV |
| NOV11f | ------------------------------------------------------------ |
| NOV11g | ------------------------------------------------------------ |
| NOV11h | ------------------------------------------------------------ |
| NOV11a | ------------------------------------------------------------ |
| NOV11b | ------------------------------------------------------------ |
| NOV11c | ------------------------------------------------------------ |
| NOV11d | ------------------------------------------------------------ |
| NOV11e | LDGTRCNTDSLDMCISCICQAVGCDRQLGSNAKEDNCGVCAGDGSTCRLVRGQSKSHVSP |
| NOV11f | ------------------------------------------------------------ |
| NOV11g | ------------------------------------------------------------ |
| NOV11h | ------------------------------------------------------------ |
| NOV11a | ------------------------------------------------------------ |
| NOV11b | ------------------------------------------------------------ |
| NOV11c | ------------------------------------------------------------ |
| NOV11d | ------------------------------------------------------------ |
| NOV11e | EKREENVIAVPLGSRSVRITVKGPAHLFIESKTLQGSKGEHSFNSPGVFVVENTTVEFQR |
| NOV11f | ------------------------------------------------------------ |
| NOV11g | ------------------------------------------------------------ |
| NOV11h | ------------------------------------------------------------ |
| NOV11a | ------------------------------------------------------------ |
| NOV11b | ------------------------------------------------------------ |
| NOV11c | ------------------------------------------------------------ |
| NOV11d | ------------------------------------------------------------ |
| NOV11e | GSERQTFKIPGPLMADFIFKTRYTAAKDSVVQFFFYQPISHQWRQTDFFPCTVTCGGGYQ |
| NOV11f | ------------------------------------------------------------ |
| NOV11g | -------------MADFIFKTRYTAAKDSVVQFFFYQPISHQWRQTDFFPCTVTCGGGYQ |
| NOV11h | ------------------------------------------------------------ |
| NOV11a | ------------------------------------------------------------ |
| NOV11b | ------------------------------------------------------------ |
| NOV11c | ------------------------------------------------------------ |
| NOV11d | --------------------------------------------------MPYDHFQPLP |
| NOV11e | LNSAECVDIRLKRVVPDHYCHYYPENVKPKPKLKECSMDPCPSSDGFKEIMPYDHFQPLP |
| NOV11f | --------------------------------------------------MPYDHFQPLP |
| NOV11g | LNSAECVDIRLKRVVPDHYCHYYPENVKPKPKLKECSMDPCPSSDGFKEIMPYDHFQPLP |
| NOV11h | ------------------------------------------------------------ |
| NOV11a | ------------------------------------------------------------ |
| NOV11b | ------------------------------------------------------------ |
| NOV11c | ------------------------------------------------------------ |
| NOV11d | RWEHNPWTACSVSCGGGIQRRSFVCVEESMHGETLQVEEWKCMYAPKPKVMQTCNLFDCP |
| NOV11e | RWEHNPWTACSVSCGGGIQRRSFVCVEESMHGEILQVEEWKCMYAPKPKVMQTCNLFDCP |
| NOV11f | RWEHNPWTACSVSCGGGIQRRSFVCVEESMHGEILQVEEWKCMYAPKPKVMQTCNLFDCP |
| NOV11g | RWENNPWTACSVSCGGGIQRRSFVCVEESMHGEILQVEEWKCMYAPKPKVMQTCNLFDCP |
| NOV11h | -WEHNPWTACSVSCGGGIQRRSFVCVEESMHGETLQVEEWKCMYAPKPKVMQTCNLFDCP |
| NOV11a | ------------------------------------------------------------ |
| NOV11b | ------------------------------------------------------------ |
| NOV11c | ------------------------------------------------------------ |
| NOV11d | KWIAMEWSQCTVTCGRGLRYRVVLCINHRGEHVGGCNPQLKLHIKEECVIPIPCYKPKEK |
| NOV11e | KWIAMEWSQCTVTCGRGLRYRVVLCINHRGEHVGGCNPQLKLHIKEECVIPIPCYKPKEK |
| NOV11f | KWIAMEWSQCTVTCGRGLRYRVVLCTNHRGEHVGGCNPQLKLHIKEECVIPIPCYKFKEK |
| NOV11g | KWIAMEWSQCTVTCGRGLRYRVVLCINHRGEHVGGCNPQLKLHIKEECVIPIPCYKPKEK |
| NOV11h | KWIANEWSQCTVTCGRGLRYRVVLCINHRGEHVGGCNPQLKLHIKEECVIPIPCYKPKEK |
| NOV11a | -----------KQPLVSSHLGIRLTSKMPQLLQNINGIIEAFRRYARTEGNCTALTRGEL |
| NOV11b | ---------------------------TGSLLQNINGIIEAFRRYARTEGNCTALTRGEL |
| NOV11c | ------------------------------------------------------------ |
| NOV11d | SPVEAKLPWLKQAQELEETRIATEEPTFIPEPWSACSTTCGPGVQVREVKCRVLLTFTQT |
| NOV11e | SPVEAKLPWLKQAQELEETRIATEEPTFIPEPWSACSTTCGPGVQVREVKCRVLLTFTQT |
| NOV11f | SPVEAKLPWLKQAQELEETRIATEEPTFIPEPWSACSTTCGPGVQVREVKCRVLLTFTQT |
| NOV11g | SPVEAKLPWLKQAQELEETRIATEEPTFIPEFWSACSTTCGPGVQVREVKCRVLLTFTQT |
| NOV11h | SPVEAKLPWLKQAQELEETRIATEEPTFIPEPWSACSTTCGPGVQVREVKCRVLLTFTQT |
| NOV11a | KRLLEQEFADVIVKPHDPATVDEVLRLLDEDHTGTVEFKEFLVLVFKVAQACFKTLSESA |
| NOV11b | KRLLEQEFADVIVKLEG------------------------------------------- |
| NOV11c | ------------------------------------------------------------ |
| NOV11d | ETELPEEECEGPKLPTERPCLLEACDESPASRELDIPLPEDSETTYDWEYAGFTPCTATC |
| NOV11e | ETELPEEECEGPKLPTERPCLLEACDESPASRELDIPLPEDSETTYDWEYAGFTPCTATC |
| NOV11f | ETELPEEECEGPKLPTERPCLLEACDESPASRELDIPLPEDSETTYDWEYAGFTPCTATC |
| NOV11g | ETELPEEECEGPKLPTERPCLLEACDESPASRELDIPLPEDSETTYDWEYAGFTPCTATC |
| NOV11h | ETELPEEECEGPKLPTERPCLLEACDESPASRELDILPLPEDSETTYDWEYAGFTFCTATC |
| NOV11a | EGACGSQESGSLNSGASQELGEGQRSGTEVGRAGKGQHYEGSSHRQSQQGSRGQNRPGVQ |
| NOV11b | ------------------------------------------------------------ |
| NOV11c | -------------------VNDSLCDMVHRPPANSQACNTEPCPPRWHVGSWGPCSATCG |
| NOV11d | VGGNQEAIAVCLHIQTQQTVNDSLCDMVNRPPANSQACNTEPCPPRWHVGSWGPCSATCG |
| NOV11e | VGGHQEAIAVCLHIQTQQTVNDSLCDMVNRPPANSQACNTEPCPPRWHVGSWGPCSATCG |
| NOV11f | VGGHQEAIAVCLNIQTQQTVNDSLCDMVHRPPANSQACNTEPCPPRWHVGSWGPCSATCG |
| NOV11g | VGGNQEAIAVCLHIQTQQTVNDSLCDMVHRPPANSQACNTEPCPPRWHVGSWGPCSATCG |
| NOV11h | VGGHQEAIAVCLHIQTQQTVNDSLCDMVHRPPANSQACNTEPCPPRWHVGSWGPCSATCG |
| NOV11a | TQGQATGSAWVSSYDRQAESQSQERISPQIQLSGQTEQTQKAGEGKRNQTTEMRPERQPQ |
| NOV11b | ------------------------------------------------------------ |
| NOV11c | VGIQTRDVYCLUPGETPAPPEECRDEKPHALQACNQFDCPPGWNIEEWQQCSRTCGGGTQ |
| NOV11d | VGIQTRDVYCLHPGETPAPPEECRDEKPHALQACNQFDCPPGWNIEEWQQCSRTCGGGTQ |
| NOV11e | VGIQTRDVYCLHPGETPAPPEECRDEKPHALQACNQFDCPPGWHIEEWQQCSRTCGGGTQ |
| NOV11f | VGIQTRDVYCLHPGETPAPPEECRDEKPNALQACNQFDCPPGWNIEEWQQCSRTCGCGTQ |
| NOV11g | VGIQTRDVYCLHPGETPAPPEECRDEKPHALQACNQFDCPPGWHIEEWGQCSRTCGGGTQ |
| NOV11h | VGIQTRDVYCLHPGETPAPPEECRDEKPHALQACNQFDCPPGWHIEEWQQCSRTCGGGTQ |
| NOV11a | TREQDRAHQTGETVTGSGTQTQAGATQTVEQDSSHQTGSTSTQTQESTNOQNRGTEIHGQ |
| NOV11b | ------------------------------------------------------------ |
| NOV11c | NR---RVTCRQLLTDGSFLNLSDELCQGPKASSHKSCARTDCPPNLAVGDWSKCSVSCGV |
| Nov11d | NR---RVTCRQLLTDGSFLNLSDELCQGPKASSHKSCARTDCPPHLAVGDWSKCSVSCGV |
| NOV11e | NR---RVTCRQLLTDGSFLNLSDELCQGPKASSNKSCARTDCPPHLAVGDWSKCSVSCGV |
| NOV11f | NR---RVTCRQLLTDGSFLNLSDELCQGPKASSNKSCARTDCPPNLAVGDWSKCSVSCGV |
| NOV11g | NR---RVTCRQLLTDGSFLNLSDELCQGPKASSHKSCARTDCPPHLAVGDWSKCSVSCGV |
| NOV11h | NR---RVTCRQLLTDGSFLNLSDELCQGPKASSHKSCARTDCPPHLAVGDWSKCSVSCGV |
| NOV11a | GRSQTSQAVTGGHTQIQAGSHTETVEQDRSQTVSHGGAREQGQTQTQPGSGQRWMQVSNP |
| NOV11b | ------------------------------------------------------------ |
| NOV11c | GIQRRKQVCQRLAAKGRRIPLSEMMCRDLPGFPLVRSCQMPECSKIKSEMKTKLGEQGPQ |
| NOV11d | GIQRRKQVCQRLAAKGRRIPLSEMMCRDLPGFPLVRSCQMPECSKIKSEMKTKLGEQGPQ |
| NOV11e | CIQRRKQVCQRLAAKGRRIPLSEMMCRDLPGFPLVRSCQMPECSKIKSEMKTKLGEQGPQ |
| NOV11f | GIQRRKQVCQRLAAKGRRIPLSEMMCRDLPGLPLVRSCQMPECSKIKSEMKTKLGEQGPQ |
| NOV11g | GIQRRKQVCQRLAAKGRRIPLSEMMCRDLPGFPLVRSCQMPECSKIKSEMKTKLGEQGPQ |
| NOV11h | GIQRRKQVCQRLAAKGRRISLSEMMCRDLPGLPLVRSCQMPEC----------------- |
| NOV11a | EAGETVPGGQAQTGASTESGRQEWSSTHPRRCVTEGQGDRQPTVVGEEWVDDHSRETVIL |
| NOV11b | ------------------------------------------------------------ |
| NOV11c | ILSVQRVYIQTREEKRINLTIGSRAYLLPNTSVIIKCPVRRFQKSLIQWEKDGRCLQNSK |
| NOV11d | ILSVQRVYIQTREEKRINLTIGSRAYLLPNTSVIIKCPVRRFQKSLIQWEKDGRCLQNSK |
| NOV11e | ILSVQRVYIQTREEKRINLTIGSRAYLLPNTSVIIKCPVRRFQKSLIQWEKDGRCLQNSK |
| NOV11f | ILSVQRVYIQTREEKRINLTIGSRAYLLPNTSVIIKCPVRRFQKSLIQWEKDGRCLQNSK |
| NOV11g | ILSVQRVYIQTREEKRINLTIGSRAYLLPNTSVIIKCPVRRFQKSLIQWEKDGRCLQNSK |
| NOV11h | ------------------------------------------------------------ |
| NOV11a | RLDQGNTLHTSVSSAQGQDAAQSEEKRGITARELYSYLRSTKP----------------- |
| NOV11b | ------------------------------------------------------------ |
| NOV11c | RLGITKSGSLKIHGLAAPDIGVYRCIAGSAQETGVLKLIGTDNRLIARPTLREPMREYPG |
| NOV11d | RLGITKSGSLKIHGLAAPDIGVYRCIAGSAQETVVLKLIGTDNRLIARPALREPMREYPG |
| NOV11e | RLGITKSGSLKIHGLAAPDIGVYRCTAGSAQETVVLKLIGTDNRLIARPALREPMREYPG |
| NOV11f | RLGITKSGSLKIHGLAAPDIGVYRCIAGSAQETVVLKLIGTDNRLIARPALREPMREYPG |
| NOV11g | RLGITKSGSLKIHGLAAPDIGVYRCIAGSAQETVVLKLIGTDNRLIARPALREPMREYPG |
| NOV11h | ------------------------------------------------------------ |
| NOV11a | ------------------------------------------------------------ |
| NOV11b | ------------------------------------------------------------ |
| NOV11c | MDHNEANS---------------------------------------------------- |
| NOV11d | MDHSEANSLGVTWHKMRQMWNNKNOLYLDDDHISNQPFLRALLGHCSNSAGSTNSWELKN |
| NOV11e | NDHSEANSLGVTWHKMRQMWNNKNDLYLDDDNISNQPFLRALLGNCSNSAGSTNSWELKN |
| NOV11f | MDHSEANSLGVTWHKMRQMWNNKNDLYLDDDHISNQPFLRALLGHCSNSAGSTNSWELKN |
| NOV11g | MDNSEANSLGVTWHKMRQMWNNKNDLYLDDDHISNQPFLRALLGHCSNSAGSTNSWELKN |
| NOV11h | ------------------------------------------------------------ |
| NOV11a | ------------------------------------------------------------ |
| NOV11b | ------------------------------------------------------------ |
| NOV11c | ------------------------------------------------------------ |
| NOV11d | KQFEAAVKQGAYSMDTAQFDELTRNMSQLMETGEVSDDLASQLIYQLVAELAKAQPTHMQ |
| NOV11e | KQFEAAVKQGAYSMDTAQFDELIRNMSQLMETGEVSDDLASQLIYQLVAELAKAQPTHMQ |
| NOV11f | KQFEAAVKQCAYSMDTAQFDELIRNMSQLMETGEVSDDLASQLIYQLVAELAKAQPTHMQ |
| NOV11g | KQFEAAVKQGAYSMDTAQFDELIRNMSQLMETGEVSDDLASQLIYQLVAELAKAQPTHMQ |
| NOV11h | ------------------------------------------------------------ |
| NOV11a | ------------------------------------------------------------ |
| NOV11b | ------------------------------------------------------------ |
| NOV11c | ------------------------------------------------------------ |
| NOV11d | WRGIQEETPPAAQLRGETGSVSQSSHAKNSGKLTFKPKGPVLMRQSQPPSTSFNKTINSR |
| NOV11e | WRGIQEETPPAAQLRGETGSVSQSSHAKNSGKLTFKPKGPVLMRQSQFPSISFNKTINSR |
| NOV11f | WRGIQEETPPAAQLRGETGSVSQSSHAKNSGKLTFKPKGPVLMRQSQPFSISFNKTINSR |
| NOV11g | WRGTQEETPPAAQLRGETGSVSQSSHAKNSGKLTFKPKGPVLMRQSQPPSISFNKTINSR |
| NOV11h | ------------------------------------------------------------ |
| NOV11a | ------------------------------------------------------------ |
| NOV11b | ------------------------------------------------------------ |
| NOV11c | ------------------------------------------------------------ |
| NOV11d | IGNTVYITKRTEVINILCDLITPSEATYTWTKDGTLLQPSVKIILDGTGKIQIQNPTRKE |
| NOV11e | IGNTVYITKRTEVINILCDLITPSEATYTWTKDGTLLQPSVKIILDGTGKIQIQNPTRKE |
| NOV11f | IGNTVYITKRTEVINILCDLITPSEATYTWTKDGTLLQPSVKIILDGTGKIQIQNPTRKE |
| NOV11g | IGNTVYITKRTEVINILCDLITPSEATYTWTKDGTLLQPSVK------------------ |
| NOV11h | ------------------------------------------------------------ |
| NOV11a | ------------------------------------------------------------ |
| NOV11b | ------------------------------------------------------------ |
| NOV11c | ------------------------------------------------------------ |
| NOV11d | QGIYECSVANHLGSDVESSSVLYAEAPVILSVERNITKPEHNHLSVVVGGIVEAALGANV |
| NOV11e | QGIYECSVANHLGSDVESSSVLYAEAPVILSVERNITKPEHNHLSVVVGGIVEAALGANV |
| NOV11f | QGIYECSVANHLGSDVESSSVLYAEAPVILSVERNITKPEHNHLSVVVGGIVEAALGANV |
| NOV11g | ------------------------------------------------------------ |
| NOV11h | ------------------------------------------------------------ |
| NOV11a | ------------------------------------------------------------ |
| NOV11b | ------------------------------------------------------------ |
| NOV11c | ------------------------------------------------------------ |
| NOV11d | TIRCPVKGVPQPNITWLKRGGSLSGNVSLLFNOSLLLQNVSLENEGTYVCIATNALGANV |
| NOV11e | TIRCPVKGVPQPNITWLKRGGSLSGNVSLLFNOSLLLQNVSLENEGTYVCIATNALGANV |
| NOV11f | TIRCPVKGVPQPNITWLKRGGSLSGNVSLLFNOSLLLQNVSLENEGTYVCIATNALGANV |
| NOV11g | ------------------------------------------------------------ |
| NOV11h | ------------------------------------------------------------ |
| NOV11a | ------------------------------------------------------------ |
| NOV11b | ------------------------------------------------------------ |
| NOV11c | ------------------------------------------------------------ |
| NOV11d | ATSVLHLLERRWPESRIVFLQGHKKYILQATNTRTNSNDPTGEPPPQEPFWEPGNWSHCS |
| NOV11e | ATSVFHLLERRWPESRIVFLQGHKKYILQATNTRTNSNDPTGEPPPQEPFWEPGNWSHCS |
| NOV11f | ATSVLHLLERRWPESRIVFLQGHKKYILQATNTRTNSNDPTGEPPPQEPFWEPGNWSHCS |
| NOV11g | ------------------------------------------------------------ |
| NOV11h | ------------------------------------------------------------ |
| NOV11a | ------------------------------------------------------------ |
| NOV11b | ------------------------------------------------------------ |
| NOV11c | ------------------------------------------------------------ |
| NOV11d | ATCGHLGARIQRPQCVMANOQEVSEALCDHLQKPLAGFEPCNIRDCPARWFTSVWSQCSV |
| NOV11e | ATCGHLGARTQRPQCVMANOQEVSEALCDHLQKPLAGFEPCNIRDCPARWFTSVWSQCSV |
| NOV11f | ATCGHLGARIQRPQCVMANOQEVSEALCDHLQKPLAGFEPCNIRDCPARWFTSVWSQCSV |
| NOV11g | ------------------------------------------------------------ |
| NOV11h | ------------------------------------------------------------ |
| NOV11a | ------------------------------------------------------------ |
| NOV11b | ------------------------------------------------------------ |
| NOV11c | ------------------------------------------------------------ |
| NOV11d | SCGEGYHSRQVTCKRTKANOTVQVVSPRACAPKDRPLGRKPCFGHPCVQWEPGNRCPGRC |
| NOV11e | SCGEGYHSRQVTCKRTKANOTVQVVSFRACAPKDRPLGRKPCFGHPCVQWEPGNRCPGRC |
| NOV11f | SCGEGYHSRQVTCKRTKANOTVQVVSPRACAPKDRPLGRKPCFGHPCVQWEPGNRCPGRC |
| NOV11g | ------------------------------------------------------------ |
| NOV11h | ------------------------------------------------------------ |
| NOV11a | ------------------------------------------------------------ |
| NOV11b | ------------------------------------------------------------ |
| NOV11c | ------------------------------------------------------------ |
| NOV11d | MGRAVRMQQRHTACQHNSSDSNCDDRKRPTLRRNCTSGACDVCWHTGPWKPCTAACGRGF |
| NOV11e | MGRAVRMQQRHTACQHNSSDSNCDDRKRPTLRRNCTSGACDVCWHTGPWKPCTAACGRGF |
| NOV11f | MGRAVRMQQRHTACQHNSSDSNCDDRKRPTLRRNCTSGACDVCWHTGPWKPCTAACGRGF |
| NOV11g | ------------------------------------------------------------ |
| NOV11h | ------------------------------------------------------------ |
| NOV11a | ------------------------------------------------------------ |
| NOV11b | ------------------------------------------------------------ |
| NOV11c | ------------------------------------------------------------ |
| NOV11d | QSRKVDCIHTRSCKPVAKRHCVQKKKPISWRHCLGPSCDRDCTDTTHYCMFVKHLNLCSL |
| NOV11e | QSRKVDCIHTRSCKPVAKRHCVQKKKPISWRHCLGPSCDRDCTDTTHYCMFVKHLNLCSL |
| NOV11f | QSRKVDCIHTRSCKPVAKRHCVQKKKPISWRHCLGPSCDRDCTDTTHYCMFVKHLNLCSL |
| NOV11g | ------------------------------------------------------------ |
| NOV11h | ------------------------------------------------------------ |
| NOV11a | -------------- |
| NOV11b | -------------- |
| NOV11c | -------------- |
| NOV11d | DRYKQRCCQSCQEG |
| NOV11e | DRYKQRCCQSCQEG |
| NOV11f | DRYKQRCCQSCQEG |
| NOV11g | -------------- |
| NOV11h | -------------- |
| NOV11a | (SEQ ID NO: 84) |
| NOV11b | (SEQ ID NO: 86) |
| NOV11c | (SEQ ID NO: 88) |
| NOV11d | (SEQ ID NO: 90) |
| NOV11e | (SEQ ID NO: 92) |
| NOV11f | (SEQ ID NO: 94) |
| NOV11g | (SEQ ID NO: 96) |
| NOV11h | (SEQ ID NO: 98) |

Further analysis of the NOV11a protein yielded the following properties shown in Table 11C.

TABLE 11C
Protein Sequence Properties NOV11a

| SignalP | No Known Signal Sequence Predicted | | | |
|---|---|---|---|---|
| analysis: | | | | |
| PSORT II | PSG: a new signal peptide prediction method | | | |
| analysis: | N-region: length 1; pos. chg 1; neg. chg 0 | | | |
| | H-region: length 10; peak value 5.49 | | | |
| | PSG score: 1.09 | | | |
| | GvH: von Heijne's method for signal seq. recognition | | | |
| | GvH score (threshold: −2.1): −10.00 | | | |
| | possible cleavage site: between 26 and 27 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 0 | | | |
| | number of TMS(s) . . . fixed | | | |
| | PERIPHERAL Likelihood = 0.63 (at 90) | | | |
| | ALOM score: 0.63 (number of TMSs: 0) | | | |
| | MTOP: Prediction of membrane topology (Hartmann et al.) | | | |
| | Center position for calculation: 6 | | | |
| | Charge difference: 0.5 C(2.5)-N(2.0) | | | |
| | C > N: C-terminal side will be inside | | | |
| | >>>Caution: Inconsistent mtop result with signal peptide | | | |
| | MITDISC: discrimination of mitochondrial targeting seq | | | |
| | R content | 1 | Hyd Moment(75): | 5.65 |
| | Hyd Moment(95): | 4.29 | G content: | 2 |
| | D/E content: | 1 | S/T content: | 4 |

Score: −4.11
Gavel: prediction of cleavage sites for mitochondrial preseq
R-3 motif at 35 FRRY|A
NUCDISC: discrimination of nuclear localization signals
pat4: none
pat7: none
bipartite: none
content of basic residues: 10.0%
NLS Score: −0.47
KDEL: ER retention motif in the C-terminus: none
ER Membrane Retention Signals:
KKXX-like motif in the C-terminus: RSTK
SKL: peroxisomal targeting signal in the C-terminus: none
PTS2: 2nd peroxisomal targeting signal: found
RLTSKMPQL at 12
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: nuclear
Reliability: 94.1
COIL: Lupas's algorithm to detect coiled-coil regions
total: 0 residues
Final Results (k = 9/23):
73.9%: nuclear
13.0%: mitochondrial
13.0%: cytoplasmic
>> prediction for CG50513-01 is nuc (k = 23)

A search of the NOV11a protein against the Geneseq database, a proprietary database that contains sequences published in patents and patent publication, yielded several homologous proteins shown in Table 11D.

TABLE 11D
Geneseq Results for NOV11a

| | | | Identities/ | |
|---|---|---|---|---|
| | | | Similarities for | |
| Geneseq | Protein/Organism/Length | NOV11a Residues/ | the Matched | Expect |
| Identifier | [Patent #, Date] | Match Residues | Region | Value |
| AAB20163 | Human protein SECP9 - Homo | 17 . . . 451 | 435/435 (100%) | 0.0 |
| | sapiens, 435 aa. [WO200105971-A2, | 1 . . . 435 | 435/435 (100%) | |
| | 25 JAN. 2001] | | | |
| AAB98668 | DRC1 protein sequence - | 17 . . . 451 | 429/495 (86%) | 0.0 |
| | Unidentified, 495 aa. [CN1283695- | 1 . . . 495 | 429/495 (86%) | |
| | A, 14 FEB. 2001] | | | |
| ABG09718 | Novel human diagnostic protein | 17 . . . 451 | 429/495 (86%) | 0.0 |
| | #9709 - Homo sapiens, 495 aa. | 1 . . . 495 | 429/495 (86%) | |
| | [WO200175067-A2, 11 OCT. 2001] | | | |
| AAU87326 | Novel central nervous system protein | 2 . . . 336 | 311/335 (92%) | e−177 |
| | #236 - Homo sapiens, 335 aa. | 1 . . . 331 | 317/335 (93%) | |
| | [WO200155318-A2, 02 AUG. 2001] | | | |
| AAU86940 | Human DNA repair and processing | 2 . . . 102 | 74/101 (73%) | 5e−31 |
| | protein, SEQ ID No 28 - Homo | 1 . . . 101 | 75/101 (73%) | |
| | sapiens, 104 aa. [WO200155305-A2, | | | |
| | 02 AUG. 2001] | | | |

In a BLAST search of public sequence databases, the NOV11a protein was found to have homology to the proteins shown in the BLASTP data in Table 11E.

TABLE 11E
Public BLASTP Results for NOV11a

| | | | Identities/ | |
|---|---|---|---|---|
| Protein | | | Similarities for | |
| Accession | | NOV11a Residues/ | the Matched | Expect |
| Number | Protein/Organism/Length | Match Residues | Portion | Value |
| CAC32430 | Sequence 17 from Patent | 17 . . . 451 | 435/435 (100%) | 0.0 |
| | WO0105971 - Homo sapiens | 1 . . . 435 | 435/435 (100%) | |
| | (Human), 435 aa. | | | |
| Q9UBG3 | Tumor related protein - Homo | 17 . . . 451 | 429/495 (86%) | 0.0 |
| | sapiens (Human), 495 aa. | 1 . . . 495 | 429/495 (86%) | |
| Q8N613 | Chromosome 1 open reading | 17 . . . 451 | 428/495 (86%) | 0.0 |
| | frame 10 - Homo sapiens | 1 . . . 495 | 428/495 (86%) | |
| | (Human), 495 aa. | | | |
| P97347 | Repetin - Mus musculus(Mouse), | 17 . . . 391 | 112/405 (27%) | 9e−27 |
| | 1130 aa. | 1 . . . 389 | 186/405 (45%) | |
| Q8VHD8 | Hornerin - Mus musculus | 17 . . . 439 | 100/431 (23%) | 1e−25 |
| | (Mouse), 2496 aa. | 1 . . . 404 | 179/431 (41%) | |

PFam analysis predicts that the NOV11a protein contains the domains shown in the Table 11F.

TABLE 11F
Domain Analysis of NOV11a

| | | Identities/ | |
|---|---|---|---|
| | NOV11a | Similarities for | |
| Pfam | Match | the Matched | Expect |
| Domain | Region | Region | Value |
| S_100 | 20 . . . 63 | 16/44 (36%) | 2.9e−09 |
| | | 34/44 (77%) | |
| efhand | 69 . . . 97 | 8/29 (28%) | 0.0061 |
| | | 23/29 (79%) | |

### Example 12

The NOV12 clone was analyzed, and the nucleotide and encoded polypeptide sequences are shown in Table 12A.

TABLE 12A
NOV12 Sequence Analysis

NOV 12a,
CG50949-03 SEQ ID NO: 103 12432 bp
DNA Sequence ORF Start: ATG at 112 ORF Stop: TAG at 1870
GGACACTGACATGGACTGAAGGAGTAGAAAACATGCCTGAGAAGCCAGGGGCCAAGATGGATCTTCTC
CTCGACATCAGCTAAGCCTGGAGGACTCTCCCCCTCAGAGACCATGGAGAGGGACAGCCACGGGAATG
CATCTCCAGCAAGAACACCTTCAGCTGGAGCATCTCCAGCCCAGGCATCTCCAGCTGGGACACCTCCA
GGCCGGGCATCTCCAGCCCAGGCATCTCCAGCCCAGGCATCTCCAGCTGGGACACCTCCGGGCCGGGC
ATCTCCAGCCCAGGCATCTCCAGCTGGTACACCTCCAGGCCGGGCATCTCCAGGCCGGGCATCTCCAG
CCCACGCATCTCCAGCCCAGGCATCTCCAGCCCGGGCATCTCCGGCTCTGGCATCACTTTCCACGTCC
TCATCCGGCACGTCATCATCCGCCAGGTCGGCCTCGGTGACAACCTCCCCAACCAGAGTGTACCTTGT
TAGAGCAACACCAGTGGGGGCTGTACCCATCCGATCATCTCCTGCCAGGTCAGCACCAGCAACCAGOG
CCACCAGGGAGAGCCCAGGTACGAGCCTGCCCAAGTTCACCTGGCGGGAGGGCCAGAAGCAGCTACCG
CTCATCGGGTGCGTGCTCCTCCTCATTCCCCTGGTGGTTTCGCTCATCATCCTCTTCCAGTTCTGGCA
GGGCCACACAGGGATCAGGTACAAGGAGCAGAGCGAGAGCTGTCCCAAGCACGCTGTTCGCTGTGACG
GGGTGGTGGACTGCAAGCTGAAGAGTGACGAGCTGGGCTGCGTGAGGTTTGACTGGGACAAGTCTCTG
CTTAAAATCTACTCTGGGTCCTCCCATCAGTGGCTTCCCATCTGTAGCAGCAACTGGAATGACTCCTA
CTCAGAGAAGACCTGCCAGCAGCTGGGTTTCGAGAGTGCTCACCGGACAACCGAGGTTGCCCACAGGG
ATTTTGCCAACAGCTTCTCAATCTTGAGATACAACTCCACCATCCAGGAAAGCCTCCACAGGTCTGAA
TGCCCTTCCCAGCGGTATATCTCCCTCCAGTGTTCCCACTGCGGACTGAGGGCCATGACCGGGCGGAT
CGTGGGAGGGGCGCTGGCCTCGGATAGCAAGTGGCCTTGGCAAGTGAGTCTGCACTTCGGCACCACCC
ACATCTGTGGAGGCACGCTCATTGACGCCCAGTGGGTGCTCACTGCCGCCCACTGCTTCTTCGTGACC
CGGGAGAAGGTCCTGGAGGGCTGCAAGGTGTACGCGGGCACCAGCAACCTGCACCAGTTGCCTGAGGC
AGCCTCCATTGCCGAGATCATCATCAACAGCAATTACACCGATGAGGAGGACGACTATGACATCGCCC
TCATGCGGCTGTCCAAGCCCCTGACCCTGTCCGCTCACATCCACCCTGCTTGCCTCCCCATGCATGGA
CAGACCTTTAGCCTCAATGAGACCTGCTGGATCACAGGCTTTGGCAACACCAGCGAGACAGATGACAA
GACATCCCCCTTCCTCCGGGAGGTGCAGGTCAATCTCATCGACTTCAAGAAATGCAATGACTACTTGG
TCTATGACAGTTACCTTACCCCAAGGATGATGTGTGCTGGGGACCTTCGTGGGGGCAGAGACTCCTGC
CAGGGAGACAGCGGGGGGCCTCTTGTCTGTGAGCAGAACAACCGCTGGTACCTGGCAGGTGTCACCAG
CTGGGGCACAGGCTGTGGCCAGAGAAACAAACCTGGTGTGTACACCAAAGTGACAGAAGTTCTTCCCT
GGATTTACAGCAAGATGGAGGTAAGATCCCTGCAGCAGGACACTGCACCCAGCAGCCTGGGAACTTCC
TCAGCTGGGGACCCTGGAGGAGCACCCAGGGTGTAGGCAGACGTCCCCTCAGCGTCCCCATATTCGGG
GGGTGTTCTGGACAGGGTCAAATGTGATGCCTGGGGTCAATCCCAGCTGTCTGTGTTTCTTTCCCTGC
TTTTCTTCCCTCAGAACAGAGCTCAGCGGCTTGAAAAAGGGTGGACCTACAGGCCAGGCAGGCAGTTG
CTGGGCAGATGTTCTCCCAGAAGTATTTTTTTGTGTAAGGTTGCAATGGACTTTGAAAACGTTTCAGT
TTCTGCAGAGGATTTTGTGATAGTCTTTGTTATCAAGCATTTATGCATGGGAATCCGCTCTTCATGGC
CTTTCCCAGCTCTGTTTGTTTTAGTCTTTTTGATTTTCTTTTTGTTGTTGTTGTTGTCTTTTTTTAAA
AACACAAGTGACTCCATTTTAACTCTGACAACTTTCACAGCTGTCACCAGAATGCTCCCTGAGAACTA
CCATTCTTTCCCTTTCCCACTTAATATTTCATCAGAACCTCACCACTATCATAAAAGAGTATATAAAG
TAATAAAATAATAAAAAGCGAAAAAAAAAAAAAAAAAAAAAAAAAAAAAA
NOV12a,
CG50949-03
Protein Sequence SEQ ID NO: 104 586 aa MW at 63152.3 kD
MERDSHGNASPARTPSAGASPAQASPAGTPPGRASPAQASPAQASPAGTPPGRASPAQASPAGTPPGR
ASPGRASPAQASPAQASPARASPALASLSRSSSGRSSSARSASVTTSPTRVYLVRATPVGAVPIRSSP
ARSAPATRATRESPGTSLPKFTWREGQKQLPLIGCVLLLIALVVSLIILFQFWQGHTGIRYKEQRESC
PKHAVRCDGVVDCKLKSDELGCVRFDWDKSLLKIYSGSSHQWLPICSSNWNDSYSEKTCQQLGFESAH
RTTEVAHRDFANSFSILRYHSTIQESLHRSECPSQRYISLQCSHCGLRANTGRIVGGALASDSKWPWQ
VSLHFGTTHICGGTLIDAQWVLTAAHCFFVTREKVLEGWKVYAGTSNLHQLPEAASIAEIIINSNYTD
EEDDYDIALMRLSKPLTLSAHIHPACLPMHGQTFSLNETCWITGFGKTRETDDKTSPFLREVQVNLID
FKKCNDYLVYDSYLTPRMMCAGDLRGGRDSCQGDSGGPLVCEQNNRWYLAGVTSWGTGCCQRNKPGVY
TKVTEVLPWIYSKMEVRSLQQDTAPSRLGTSSGCDPGGAPRV
NOV12b,
197192399 SEQ ID NO: 105 717 bp
DNA Sequence ORF Start: at 1 ORF Stop: end of sequence
TCCCTGTGGATTGGATCCATCGTGGGAGGGGCGCTGGCCTCGGATAGCAAGTGGCCTTGGCAAGTGAG
TCTGCACTTCGGTACCACCCACATCTGTGGAGGCACGCTCATTGACGCCCAGTGGGTGCTCACTCCCG
CCCACTGCTTCTTCGTGACCCGGGAGAAGGTCCTGGAGGGCTGGAAGGTGTACGCGGGCACCAGCAAC
CTGCACCAGTTGCCTGAGGCAGCCTCCATTGCCGAGATCATCATCAACAGCAATTACACCGATGAGGA
GGACGACTATGACATCGCCCTCATGCGGCTGTCCAAGCCCCTGACCCTGTCCGCTCACATCCACCCTG
CTTGCCTCCCCATGCATGGACAGACCTTTAGCCTCAATGAGACCTGCTGGATCACAGGCTTTGGCAAG
ACCAGGGAGACAGATGACAAGACATCCCCCTCCCTCCGGGAGGTGCAGGTCAATCTCATCGACTTCAA
GAAATGCAATGACTACTTGGTCTATCACAGTTACCTTACCCCAAGGATGATGTGTGCTGGGGACCTTC
GTGGGGGCAGAGACTCCTGCCAGGGAGACACCGGGGGGCCTCTTGTCTGTGAGCAGAACAACCGCTGG
TACCTGGCAGGTGTCACCAGCTGCGGCACACGCTGTGGCCAGAGAAACAAACCTGGTGTGTACACCAA
AGTCACAGAAGTTCTTCCCTGGATTTACAGCCTCGAG
NOV12b,
197192399
Protein Sequence SEQ ID NO: 106 239 aa MW at 26529.8 kD
SLWIGSIVGGALASDSKWPWQVSLHFGTTHICGGTLIDAQWVLTAAHCFFVTREKVLEGWKVYAGTSN
LHQLPEAASIAEIIINSNYTDEEDDYDIALMRLSKPLTLSAHIHPACLPMHGQTFSLNETCWITGFGK
TRETDDKTSPSLREVQVNLIDFKKCNDYLVYDSYLTPRMMCAGDLRGGRDSCQGDSGGPLVCEQNNRW
YLAGVTSWGTGCGQRNKPGVYTKVTEVLPWIYSLE
NOV12c,
257499999 SEQ ID NO: 107 717 bp
DNA Sequence ORF Start: at 1 ORF Stop: end of sequence
TCCCTGTGGATTGGATCCATCGTCGGAGGGGCGCTGGCCTCGGATAGCAAGTGGCCTTGGCAAGTGAG
TCTGCACTTCGGTACCACCCACATCTGTGGAGGCACGCTCATTGACGCCCAGTGGGTGCTCACTGCCG
CCCACTGCTTCTTCGTGACCCGGGAGAAGCTCCTGGAGGGCTGGAAGGTGTACGCGGGCACCAGCAAC
CTGCACCAGTTGCCTGAGGCAGCCTCCATTGCCGAGATCATCATCAACAGCAATTACACCGATGAGGA
GGACGACTATGACATCGCCCTCATGCGGCTGTCCAAGCCCCTGACCCTGTCCGCTCACATCCACCCTG
CTTGCCTCCCCATGCATGGACAGACCTTTAGCCTCAATGAGACCTGCTGGATCACAGGCTTTGGCAAG
ACCAGGGAGACAGATGACAAGACATCCCCCTCCCTCCGGGAGGTGCAGGTCAATCTCATCGACTTCAA
GAAATGCAATGACTACTTGGTCTATGACAGTTACCTTACCCCAAGGATGATGTGTGCTGGGGACCTTC
GTGGGGGCAGAGACTCCTGCCAGGGAGACAGCGGGGGGCCTCTTGTCTGTGAGCAGAACAACCCCTGG
TACCTGGCAGGTGTCACCAGCTGGGGCACAGGCTGTGGCCAGAGAAACAAACCTGGTGTGTACACCAA
AGTGACAGAAGTTCTTCCCTGGATTTACAGCCTCGAG
NOV12c,
257499999
Protein Sequence SEQ ID NO: 108 1239 aa MW at 26529.8 kD
SLWIGSLVGGALASDSKWPWQVSLHFGTTHICGGTLIDAQWVLTAAHCFFVTREKVLEGWKVYAGTSN
LUQLPEAASIAEIIINSNYTDEEDDYDIALMRLSKPLTLSAHIHPACLPMHGQTFSLNETCWITGFCK
TRETDDKTSPSLREVQVNLIDFKKCNDYLVYDSYLTPRMMCAGDLRGGRDSCQGDSGGPLVCEQNNRW
YLAGVTSWGTGCGQRNKPGVYTKVTEVLPWIYSLE
NOV12d,
257450010 SEQ ID NO: 109 1101 bp
DNA Sequence ORF Start: at 1 ORF Stop: end of sequence
GGATCCACAGGGATCAGGTACAAGGAGCAGAGGGAGAGCTGTCCCAAGCACGCTGTTCGCTGTGACGG
GGTGGTGGACTGCAAGCTGAAGAGTGACGAGCTGGGCTGCGTGAGGTTTGACTGGGACAAGTCTCTGC
TTAAAATCTACTCTGUGTCCTCCCATCAGTGGCTTCCCATCTGTAGCAGCAACTGGAATGACTCCTAC
TCAGAGAAGACCTGCCAGCAGCTGGGTTTCGAGAGGTCTGAATGCCCTTCCCAGCGGTATATCTCCCT
CCAGTGTTCCCACTGCGGACTGAGGGCCATGACCGGGCGGATCGTGGGAGGGGCGCTGGCCTCGGATA
GCAAGTGGCCTTGGCAAGTGAGTCTGCACTTCGGCACCACCCACATCTGTGGAGGCACGCTCATTGAC
GCCCAGTGGGTGCTCACTCCCGCCCACTGCTTCTTCGTGACCCGGGAGAAGGTCCTGGAGGGCTGGAA
GCTGTACCCGGGCACCAGCAACCTGCACCAGTTGCCTGAGGCAGCCTCCATTGCCCAGATCATCATCA
ACAGCAATTACACCGATGAGGAGGACGACTATGACATCGCCCTCATGCGGCTGTCCAAGCCCCTGACC
CTGTCCGCTCACATCCACCCTGCTTGCCTCCCCATGCATGGACAGACCTTTAGCCTCAATGAGACCTG
CTGGATCACAGGCTTTGGCAAGACCAGGGAGACAGATGACAAGACATCCCCCTTCCTCCGGGAGGTGC
AGGTCAATCTCATCGACTTCAAGAAATGCAATGACTACTTGGTCTATGACAGTTACCTTACCCCAAGG
ATGATGTGTGCTGGGGACCTTCGTGGGGGCAGAGACTCCTGCCAGGGAGACAGCGGGGGGCCTCTTGT
CTGTGAGCAGAACAACCGCTGGTACCTGGCAGGTGTCACCAGCTGGGGCACAGGCTGTGGCCAGAGAA
ACAAACCTGGTGTGTACACCAAAGTGACAGAAGTTCTTCCCTGGATTTACAGCAAGATGGAGGTAAGA
TCCCTGCAGCAGGACACTGCACCCAGCAGGCTGGGAACTTCCTCAGGTGGGGACCCTGGAGGAGCACC
CAGGGTGCTCGAG
NOV12d,
257450010
Protein Sequence SEQ ID NO: 110 367 aa MW at 40822.7 kD
GSTGIRYKEQRESCPKHAVRCDGVVDCKLKSDELGCVRFDWDKSLLKIYSGSSHQWLPICSSNWNDSY
SEKTCQQLGFERSECPSQRYISLQCSHCGLRAMTGRIVGGALASDSKWPWQVSLHFGTTHICGGTLID
AQWVLTAAHCFFVTREKVLEGWKVYAGTSNLHQLPEAASIAEIIINSNYTDEEDDYDIALMRLSKPLT
LSAHIHPACLPMHGQTFSLNETCWITGFGKTRETDDKTSPFLREVQVNLIDFKKCNDYLVYDSYLTPR
MMCAGDLRGGRDSCQGDSGGPLVCEQNNRWYLAGVTSWGTGCGQRNKPGVYTKVTEVLPWIYSKMEVR
SLQQDTAPSRLGTSSGGDPGGAPRVLE
NOV12e,
252417780 SEQ ID NO: 111 1203 bp
DNA Sequence ORF Start: at ORF Stop: end of sequence
ACCGGATCCCACACAGGGATCAGGTACAAGGAGCAGAGGGAGAGCTGTCCCAAGCACGCTGTTCGCT
GTGACGGGGTGGTGGACTGCAAGCTGAAGAGTGACGAGCTGGGCTGCGTGAGGTTTGACTGGGACAAG
TCTCTGCTTAAAATCTACTCTGGOTCCTCCCATCAGTGGCTTCCCATCTGTAGCAGCAACTGGAATGA
CTCCTACTCAGAGAAGACCTGCCAGCAGCTGGGTTTCGAGAGTGCTCACCGGACAACCGAGGTTGCCC
ACAGGGATTTTGCCAACAGCTTCTCAATCTTGAGATACAACTCCACCATCCAGGAAAGCCTCCACAGG
TCTGAATGCCCTTCCCAGCGGTATATCTCCCTCCAGTGTTCCCACTGCGGACTGAGGGCCATGACCGG
GCGGATCGTGGGAGGGGCGCTGCCCTCGGATAGCAAGTGGCCTTGGCAAGTGAGTCTGCACTTCGGCA
CCACCCACATCTGTGGAGGCACGCTCATTGACGCCCAGTGGGTGCTCACTGCCGCCCACTGCTTCTTC
GTGACCCGGGAGAAGGTCCTGGAGGGCTGGAAGGTGTACGCCGGCACCAGCAACCTGCACCAGTTGCC
TGAGGCAGCCTCCATTGCCGAGATCATCATCAACAGCAATTACACCGATGAGGAGGACGACTATGACA
TCGCCCTCATGCGGCTGTCCAAGCCCCTGACCCTGTCCGCTCACATCCACCCTGCTTGCCTCCCCATG
CATGGACAGACCTTTAGCCTCAATGAGACCTGCTGCATCACAGCCTTTGGCAAGACCAGGGAGACAGA
TGACAAGACATCCCCCTTCCTCCGGGAGCTGCAGGTCAATCTCATCGACTTCAAGAAATGCAATCACT
ACTTGGTCTATGACAGTTACCTTACCCCAAGGATGATGTGTCCTGGGGACCTTCGTGGGGGCAGAGAC
TCCTGCCAGGGAGACAGCGGGGGGCCTCTTGTCTGTGAGCAGAACAACCGCTGGTACCTGGCAGGTGT
CACCAGCTGGGGCACAGGCTGTGGCCAGAGAAACAAACCTGGTGTGTACACCAAAGTGACAGAAGTTC
TTCCCTGGATTTACAGCAAGATGGAGGTAAGATCCCTGCAGCAGGACACTGCACCCAGCAGGCTGGGA
ACTTCCTCAGCTGGGGACCCTCGAGGAGCACCCAGGGTGCTCGAGGGC
NOV 12e,
252417780
Protein Sequence SEQ ID NO: 112 401 aa MW at 44688.8 kD
TGSHTGIRYKEQRESCPKHAVRCDGVVDCKLKSDELGCVRFDWDKSLLKIYSGSSHQWLPICSSNWND
SYSEKTCQQLGFESAHRTTEVAHRDFANSFSILRYNSTIQESLHRSECPSQRYISLQCSNCGLRAMTG
RIVGGALASDSKWPWQVSLHFGTTHICGGTLIDAQWVLTAAHCFFVTREKVLEGWKVYAGTSNLHQLP
EAASIAEIIINSNYTDEEDDYDIALMRLSKPLTLSAHIHPACLPMHGQTFSLNETCWITGFGKTRETD
DKTSPFLREVQVNLIDFKKCNDYLVYDSYLTPRMMCAGDLRGGRDSCQGDSGGPLVCEQNNRWYLAGV
TSWGTGCGQRNKPGVYTKVTEVLPWIYSKMEVRSLQQDTAPSRLGTSSGGDPGGAPRVLEG
NOV12f,
252417791 SEQ ID NO: 113 1110 bp
DNA Sequence ORF Start: at 2 ORF Stop: end of sequence
ACCGGATCCCACACAGGGATCAGGTACAAGGAGCAGAGGGAGAGCTGTCCCAAGCACGCTGTTCGCT
GTGACGGGGTGGTGGACTGCAAGCTGAAGAGTGACGAGCTGGGCTGCGTGAGGTTTGACTGGGACAAG
TCTCTGCTTAAAATCTACTCTGGGTCCTCCCATCAGTGGCTTCCCATCTGTAGCACCAACTGGAATGA
CTCCTACTCAGAGAAGACCTGCCAGCAGCTGGGTTTCGAGAGGTCTGAATGCCCTTCCCAGCGGTATA
TCTCCCTCCAGTGTTCCCACTGCGGACTGAGGGCCATGACCGGGCGGATCGTGGGAGGGGCGCTGGCC
TCGGATAGCAAGTGGCCTTGGCAAGTGAGTCTGCACTTCGGCACCACCCACATCTGTGCAGGCACGCT
CATTGACCCCCAGTGGGTGCTCACTGCCGCCCACTGCTTCTTCGTGACCCGGGAGAAGGTCCTGGAGG
GCTGGAAGGTGTACGCGGGCACCAGCAACCTGCACCAGTTGCCTGAGCCAGCCTCCATTGCCGAGATC
ATCATCAACAGCAATTACACCGATGACGAGGACGACTATGACATCGCCCTCATGCGGCTGTCCAAGCC
CCTGACCCTGTCCGCTCACATCCACCCTGCTTGCCTCCCCATGCATGGACAGACCTTTAGCCTCAATG
AGACCTCCTGGATCACAGGCTTTGGCAAGACCAGGGAGACAGATGACAAGACATCCCCCTTCCTCCGG
GAGGTGCAGGTCAATCTCATCCACTTCAAGAAATGCAATGACTACTTGGTCTATGACAGTTACCTTAC
CCCAAGGATGATGTGTGCTGGGGACCTTCGTGGGGGCAGAGACTCCTGCCAGGGAGACAGCGGGGGGC
CTCTTGTCTGTGAGCAGAACAACCGCTGGTACCTGGCAGGTGTCACCAGCTGGGGCACAGGCTGTGGC
CAGAGAAACAAACCTGGTGTGTACACCAAAGTGACAGAAGTTCTTCCCTGCATTTACAGCAAGATGGA
GGTAAGATCCCTGCAGCAGCACACTGCACCCAGCAGGCTGGGAACTTCCTCAGGTGGGGACCCTGGAG
GAGCACCCAUGGTGCTCGAGGGC
NOV12f,
252417791
Protein Sequence SEQ ID NO: 114 370 aa MW at 41118.0 kD
TGSHTGIRYKEQRESCPKHAVRCDGVVDCKLKSDELGCVRFDWDKSLLKIYSGSSHQWLPICSSNWND
SYSEKTCQQLGFERSECPSQRYISLQCSHCGLRAMTGRIVGGALASKSKWPWQVSLHFGTTHICGGTL
IDAQWVLTAAHCFFVTREKVLEGWKVYAGTSNLHQLPEAASIAEIIINSNYTDEEDDYDIALMRLSKR
LTLSAHIHPACLPMHGQTFSLNETCWITGFGKTRETDDKTSPFLREVQVNLIDFKKCNDYLVYDSYLT
PRMNCAGDLRGGRDSCQGDSGGPLVCEQNNRWYLAGVTSWGTGCGQRNKPGVYTKVTEVLPWIYSKME
VRSLQQDTAPSRLGTSSGGDPGGAPRVLEG
NOV12g,
252417821 SEQ ID NO: 115 1203 bp
DNA Sequence ORF Start: at 2 ORF Stop: end of sequence
ACCGGATCCCACACAGGGATCAGGTACAAGGAGCAGAGGGAGAGCTGTCCCAAGCACGCTGTTCGCT
GTGACGGGGTGGTGGACTGCAAGCTGAAGAGTGACGAGCTGGGCTGCGTGAGGTTTGACTGGGACAAC
TCTCTGCTTAAAATCTACTCTGGGTCCTCCCATCAGTGGCTTCCCATCTGTAGCAGCAACTGGAATGA
CTCCTACTCAGAGAAGACCTGCCAGCAGCTGGGTTTCGAGAGTGCTCACCGGACAACCGACGTTGCCC
ACAGGGATTTTGCCAACAGCTTCTCAATCTTGAGATACAACTCCACCATCCAGGAAAGCCTCCACAGG
TCTGAATGCCCTTTCCAGCGGTATATCTCCCTCCAGTGTTCCCACTGCGGACTGAGGGCCATGACCGG
GCGGATCGTGGGAGGGGCGCTGGTCTCGGATAGCAAGTGGCCTTGGCAAGTGAGTCTGCACTTCGGCA
CCACCCACATCTGTGGAGGCACGCTCATTGACGCCCAGTGGGTGCTCACTGCCGCCCACTGCTTCTTC
GTGACCCGGCAGAAGGTCCTGGAGGGCTGGAAGGTGTACGCGGGCACCAGCAACCTGCACCAGTTGCC
TGAGGCAGCCTCCATTGCCGAGATCATCATCAACAGCAATTACACCGATGAGGAGGACGACTATGACA
TCGCCCTCATGCGGCTGTCCAACCCCCTGACCCTGTCCGCTCACATCCACCCTGCTTGCCTCCCCATG
CATGGACAGACCTTTAGCCTCAATGAGACCTGCTGGATCACAGGCTTTGGCAAGACCAGGGAGACACA
TGACAAGACATCCCCCTTCCTCCGGGAGGTGCAGGTCAATCTCATCGACTTCAAGAAATGCAATGACT
ACTTGGTCTATGACAGTTACCTTACCCCAAGGATGATGTGTGCTCGGGACCTTCGTGGGGGCAGAGAC
TCCTGCCAGGGAGACAGCGGGGGGCCTCTTGTCTGTGAGCAGAACAACCGCTGGTACCTGGCAGGTGT
CACCAGCTGGGGCACAGGCTCTGGCCAGAGAACAAACCTGGTGTGTACACCAAAGTGACAGAAGTTC
TTCCCTGGATTTACAGCAAGATGGAGGTAAAATCCCTGCAGCAGGACACTGCACCCAGCAGGCTGGGA
ACTTCCTCAGGTGGGGACCCTGGAGGAGCACCCAGGGTGCTCGAGGGC
NOV12g,
252417821
Protein Sequence SEQ ID NO: 116 401 aa MW at 44749.0 kD
TGSHTGIRYKEQRESCPKHAVRCDGVVDCKLKSDELGCVRFDWDKSLLKIYSGSSHQWLPICSSNWND
SYSEKTCQQLGFESAHRTTEVAHRDFANSFSILRYNSTIQESLHRSECPFQRYISLQCSHCGLRAMTG
RIVGGALVSDSKWPWQVSLHFGTTHICGGTLIDAQWVLTAAHCFFVTREKVLEGWKVYAGTSNLHQLP
EAASIAEIIINSNYTDEEDDYDIALMRLSKPLTLSAHIHPACLPMHGQTFSLNETCWITGFGKTRETD
DKTSPFLREVQVMLIDFKKCNDYLVYDSYLTPRMMCAGDLRGGRDSCQGDSGGPLVCEQNNRWYLAGV
TSWGTGCGQRNKPGVYTKVTEVLPWIYSKMEVKSLQQDTAPSRLGTSSGGDPGGAPRVLEG
NOV12h,
252417840 SEQ ID NO: 117 1203 bp
DNA Sequence ORF Start: at 1 ORF Stop: end of sequence
ACCGGATCCCACACAGGGATCAGGTACAAGGAGCAGAGGGAGAGCTGTCCCAAGCACGCTGTTCGCT
CTGACGGGGTGGTGGACTGCAAGCTGAAGAGTGACGAGCTGGGCTGCCTGAGGTTTGACTGGGACAAG
TCTCTGCTTAAAATCTACTCTGGGTCCTCCCATCAGTGGCTTCCCATCTGTAGCAGCAACAGCAATGA
CTCCTACTCAGAGAAGACCTGCCAGCAGCTGGGTTTCGAGAGTGCTCACCGGACAACCGAGGTTGCCC
ACAGGGATTTTCCCAACAGCTTCTCAATCTTGACATACAACTCCACCATCCAGGAAAGCCTCCACAGG
TCTGAATGCCCTTCCCAGCGGTATATCTCCCTCCAGTGTTCCCACTGCGGACTGAGGGCCATGACCGG
GCGGATCGTGGGAGGGGCGCTGGCCTCGGATAGCAAGTGGCCTTGGCAAGTGAGTCTGCACTTCGGCA
CCACCCACATCTGTGGAGGCACGCTCATTGACGCCCAGTGGGTGCTCACTGCCGCCCACTGCTTCTTC
GTGACCCGGGAGAAGGTCCTGGAGGGCTGGAAGGTGTACGCGGGCACCAGCAACCTGCACCAGTTGCC
TGACGCAGCCTCCATTGCCGAGATCATCATCAACAGCAATTACACCGATGAGGAGGACGACTATGACA
TCGCCCTCATGCGGCTGTCCAAGCCCCTGACCCTGTCCGCTCACATCCACCCTGCTTGCCTCCCCATG
CATGGACAGACCTTTAGCCTCAATGAGACCTGCTGGATCACAGGCTTTGGCAAGACCAGGGAGACAGA
TGACAACACATCCCCCTTCCTCCGGGAGGTGCAGGTCAATCTCATCGACTTCAAGAAATGCAATGACT
ACTTGGTCTATGACAGTTACCTTACCCCAAGGATGATGTGTGCTCGGGACCTTCGTGGGGGCAGAGAC
TCCTGCCAGGGAGACAGCGGGCGGCCTCTTGTCTGTGAGCAGAACAACCGCTGGTACCTGGCAGGTGT
CACCAGCTGGGGCACAGGCTGTGGCCAGAGAAACAAACCTGGTGTGTACACCAAAGTGACAGAAGTTC
TTCCCTGGATTTACAGCAAGATGGAGGTAAGATCCCTGCAGCAGGACACTGCACCCAGCAGGCTGGGA
ACTTCCTCAGGTGGGGACCCTGGAGGAGCACCCAGGGTGCTCGAGGGC
NOV12h,
252417840 SEQ ID NO: 118 401 aa MW at 44658,8 kD
TGSHTGIRYKEQRESCPKHAVRCDGVVDCKLKSDELGCVRFDWDKSLLKIYSGSSHQWLPICSSNRND
SYSEKTCQQLQFESAHRTTEVAHRDFANSFSILRYNSTIQESLHRSECPSQRYISLQCSHCGLRAMTG
RIVGGALASDSKWPWQVSLHFGTTHICGGTLIDAQWVLTAAHCFFVTREKVLEGWKVYAGTSNLHQLP
EAASLAEIIIUSNYTDEEDDYDIALMRLSKPLTLSAHIHPACLPMHGQTFSLNETCWITGFGKTRETD
DKTSPFLREVQVNLIDFKKCNDYLVYDSYLTPRMMCAGDLRGGRDSCQGDSGGPLVCEQNNRWYLAGV
TSWGTGCGQRNKPGVYTKVTEVLPWIYSKNEVRSLQQDTAPSRLGTSSGGDPGGAPRVLEG
NOW 12i,
257474313 SEQ ID NO: 19 1779 bp
DNA Sequence ORF Start: at 1 ORF Stop: end of sequence
ACCGGATCCACCATCGAGAGGGACAGCCACGGGAATGCATCTCCAGCAAGAACACCTTCAGCTGGAG
CATCTCCAGCCCAGGCATCTCCAGCTGGGACACCTCCAGGCCGGGCATCTCCAGCCCAGGCATCTCCA
GCCCAGGCATCTCCAGCTGGCACACCTCCGGGCCGGGCATCTCCAGCCCAGGCATCTCCAGCTGGTAC
ACCTCCAGGCCGGGCATCTCCAGGCCGGGCATCTCCAGCCCAGGCATCTCCAGCCCAGGCATCTCCAG
CCCGGGCATCTCCGGCTCTGGCATCACTTTCCAGGTCCTCATCCGGCAGGTCATCATCCGCCAGGTCA
GCCTCGGTGACAACCTCCCCAACCAGAGTGTACCTTGTTAGACCAACACCAGTGGGGGCTGTACCCAT
CCGATCATCTCCTGCCAGGTCAGCACCAGCAACCAGGGCCACCAGGGAGAGCCCAGGTACGAGCCTGC
CCAAGTTCACCTGGCGGGAGGGCCAGAAGCAGCTACCGCTCATCGGGTGCGTGCTCCTCCTCATTGCC
CTGGTGGTTTCGCTCATCATCCTCTTCCAGTTCTGGCAGGGCCACACAGGGATCAGGTACAAGGAGCA
GAGGGAGAGCTGTCCCAACCACGCTGTTCGCTGTGACGGGGTGGTGGACTGCAAGCTGAAGAGTGACG
AGCTGGGCTGCGTGAGGTTTGACTGGGACAAGTCTCTGCTTAAAATCTACTCTGGGTCCTCCCATCAG
TGGCTTCCCATCTGTAGCAGCAACTGGAATGACTCCTACTCAGAGAAGACCTGCCAGCAGCTGGGTTT
CGAGAGTGCTCACCGGACAACCGAGGTTGCCCACAGGGATTTTGCCAACAGCTTCTCAATCTTGAGAT
ACAACTCCACCATCCAGGAAAGCCTCCACAGGTCTGAATGCCCTTCCCAGCGGTATATCTCCCTCCAG
TGTTCCCACTGCGGACTGAGGGCCATGACCGGGCGGATCGTGGGAGGGGCGCTGGCCTCGGATAGCAA
GTGGCCTTGGCAAGTGAGTCTGCACTTCGGCACCACCCACATCTGTGGAGGCACGCTCATTGACGCCC
AGTGGGTGCTCACTGCCGCCCACTGCTTCTTCGTGACCCGGGAGAAGGTCCTGGAGGGCTGGAAGGTG
TACGCGGGCACCAGCAACCTGCACCAGTTGCCTGAGGCAGCCTCCATTGCCGAGATCATCATCAACAG
CAATTACACCGATGAGGAGGACGACTATGACATCGCCCTCATGCGGCTGTCCAAGCCCCTGACCCTGT
CCGCTCACATCCACCCTGCTTGCCTCCCCATGCATGGACAGACCTTTAGCCTCAATGAGACCTGCTGG
ATCACAGGCTTTGGCAAGACCAGGGAGACAGATGACAAGACATCCCCCTTCCTCCGGGAGGTGCAGGT
CAATCTCATCGACTTCAACAAATGCAATGACTACTTCGTCTATGACAGTTACCTTACCCCAAGGATGA
TGTGTGCTGGGGACCTTCGTGGGCGCAGAGACTCCTGCCAGGGAGACAGCGGGGGGCCTCTTGTCTGT
GAGCAGAACAACCGCTGGTACCTGGCAGGTGTCACCAGCTCGGGCACAGGCTGTGGCCAGAGAAACAA
ACCTGGTGTGTACACCAAAGTGACAGAAGTTCTTCCCTGGATTTACAGCAAGATGGAGGTAAGATCCC
TGCAGCAGGACACTGCACCCACCAGGCTGGGAACTTCCTCAGGTGGGGACCCTGGAGGAGCACCCAGG
GTGCTCGAGGGC
NOV12i,
257474313
Protein Sequence SEQ ID NO: 120 593 aa MW at 63798.0 kD
TGSTMERDSHGNASPARTPSAGASPAQASPAGTPPGRASPAQASPAQASPAGTPPURASPAQASPAGT
PPGRASPGRASPAQASPAQASPARASPALASLSRSSSGRSSSARSASVTTSPTRVYLVRATPVGAVPI
RSSPARSAPATRATRESPGTSLPKFTWREGQKQLPLIGCVLLLTALVVSLIILFQFWQGHTGIRYKEQ
RESCPKHAVRCDGVVDCKLKSDELGCVRFDWDKSLLKIYSGSSHQWLPICSSNWNDSYSEKTCQQLGF
ESAHRTTEVAHRDFANSFSILRYNSTIQESLHRSECPSQRYISLQCSHCGLRAMTGRIVGGALASDSK
WPWQVSLHFGTTHICGGTLIDAQWVLTAAHCFFVTREKVLECWKVYAGTSNLHQLPEAASIAEIIINS
NYTDEEDDYDIALMRLSKPLTLSAHIHPACLPMHGQTFSLNETCWITGFGKTRETDDKTSPFLREVQV
NLIDFKKCNDYLVYDSYLTPRMMCAGDLRGGRDSCQGDSGGPLVCEQNNRWYLAGVTSWGTGCGQRNK
PGVYTKVTEVLPWIYSKMEVRSLQQDTAPSRLGTSSGGDPGGAPRVLEG
NOV12j,
257474324 SEQ ID NO: 121 1626 bp
DNA Sequence ORF Start: at 1 ORF Stop: end of sequence
ACCGGATCCACCATGGAGAGGGACAGCCACGGGAATGCATCTCCAGCAAGAACACCTTCAGCTGGAG
CATCTCCAGCCCAGGCATCTCCAGCTGGGACACCTCCAGGCCGCGCATCTCCAGCCCAGGCATCTCCA
GCCCAGGCATCTCCAGCTGGGACACCTCCGGGCCGGGCATCTCCAGCCCAGGCATCTCCAGCTGGTAC
ACCTCCAGGCCGGGCATCTCCGGCTCTGGCATCACTTTCCAGGTCCTCATCCGGCAGGTCATCATCCG
CCAGGTCAGCCTCGGTGACAACCTCCCCAACCAGAGTGTACCTTGTTAGAGCAACACCAGTGGGGGCT
GTACCCATCCGATCATCTCCTGCCAGCTCAGCACCAGCAACCAGGGCCACCAGGGAGACCCCAGGTAC
GAGCCTGCCCAAGTTCACCTGGCGGGAGGGCCAGAAGCAGCTACCGCTCATCGGGTGCGTGCTCCTCC
TCATTGCCCTGGTGGTTTCGCTCATCATCCTCTTCCAGTTCTGGCAGGGCCACACAGGGATCAGGTAC
AACGAGCAGAGGGAGAGCTGTCCCAAGCACGCTGTTCGCTGTGACGGGGTGGTGGACTGCAAGCTGAA
GAGTGACGAGCTGGGCTGCGTGAGGTTTCACTGGGACAAGTCTCTGCTTAAAATCTACTCTGGGTCCT
CCCATCAGTGGCTTCCCATCTGTAGCAGCAACTGGAATGACTCCTACTCAGAGAAGACCTGCCAGCAG
CTGGGTTTCGAGAGGTCTGAATGCCCTTCCCAGCGGTATATCTCCCTCCAGTGTTCCCACTGCGGACT
GAGGGCCATGACCGGGCGGATCGTGGGAGGGGCGCTGGCCTCGGATAGCAAGTGGCCTTGGCAAGTGA
GTCTGCACTTCGGCACCACCCACATCTUTGGAGGCACGCTCATTGACGCCCAGTGGGTGCTCACTGCC
GCCCACTGCTTCTTCGTGACCCUGGAGAAGGTCCTGGACGGCTGGAAGGTGTACGCGGGCACCAGCAA
CCTGCACCAGTTGCCTGAGGCAGCCTCCATTGCCGAGATCATCATCAACAGCAATTACACCGATGAGG
AGGACGACTATGACATCGCCCTCATGCGGCTGTCCAAGCCCCTGACCCTGTCCGCTCACATCCACCCT
GCTTGCCTCCCCATGCATGGACAGACCTTTAGCCTCAATGAGACCTGCTGGATCACAGGCTTTGGCAA
GACCAGGGAGACAGATGACAAGACATCCCCCTTCCTCCGGGAGGTGCAGGTCAATCTCATCGACTTCA
AGAAATGCAATGACTACTTGGTCTATGACAGTTACCTTACCCCAAGGATGATGTGTGCTGGGGACCTT
CGTGGGGGCAGAGACTCCTGCCAGGGAGACAGCGGGGGGCCTCTTGTCTGTGACCAGAACAACCGCTG
GTACCTGCCAGGTGTCACCAGCTGGGGCACAGGCTGTGGCCAGAGAAACAAACCTGGTGTGTACACCA
AAGTGACAGAAGTTCTTCCCTGGATTTACAGCAAGATGGAGGTAAGATCCCTGCAGCAGGACACTGCA
CCCAGCAGGCTGGGAACTTCCTCAGGTGGGGACCCTGGAGGAGCACCCAGGGTGCTCGAGGGC
NOV12j,
257474324
Protein Sequence SEQ ID NO: 122 542 aa MW at 58367.2 kD
TGSTMERDSHGNASPARTPSAGASPAQASPAGTPPGRASPAQASPAQASPAGTPPGRASPAQASPAGT
PPGRASPALASLSRSSSCRSSSARSASVTTSPTRVYLVRATPVGAVPIRSSPARSAPATRATRESPGT
SLPKFTWREGQKQLPLIGCVLLLIALVVSLIILFQFWQGGTGIRYKEQRESCPKHAVRCDGVVDCKLK
SDELGCVRFDWDKSLLKIYSGSSHQWLPICSSNWNDSYSEKTCQQLGFERSECPSQRYISLQCSHCGL
RAMTGRIVGGALASDSKWPWQVSLHFGTTHICGGTLIDAQWVLTAAHCFFVTREKVLEGWKVYAGTSN
LHQLPEAASIAEIIINSNYTDEEDDYDIALMRLSKPLTLSAHIHPACLPMHGQTFSLNETCWITGFGK
TRETDDKTSPFLREVQVNLIDFKKCNDYLVYDSYLTPRMMCAGDLRGGRDSCQGDSGGPLVCEQNNRW
YLAGVTSWGTCCGQRNKPGVYTKVTEVLPWIYSKMEVRSLQQDTAPSRLGTSSGGDPGGAPRVLEG
NOV12k,
CG50949-06 SEQ ID NO: 123 1780 bp
DNA Sequence ORF Start: ATG at 14 ORF Stop: end of sequence
CACCGGATCCACCATGGAGAGGGACAGCCACCGGAATGCATCTCCAGCAAGAACACCTTCAGCTGGAG
CATCTCCAGCCCAGGCATCTCCAGCTGCGACACCTCCAGGCCGGGCATCTCCAGCCCAGGCATCTCCA
GCCCAGGCATCTCCAGCTGGGACACCTCCGGGCCGGGCATCTCCAGCCCAGGCATCTCCAGCTGGTAC
ACCTCCAGGCCGGGCATCTCCAGGCCGGGCATCTCCAGCCCAGGCATCTCCAGCCCAGGCATCTCCAG
CCCGGGCATCTCCGGCTCTGGCATCACTTTCCAGGTCCTCATCCGGCAGGTCATCATCCGCCAGGTCA
GCCTCGGTCACAACCTCCCCAACCAGAGTGTACCTTGTTAGAGCAACACCAGTGGGGGCTGTACCCAT
CCGATCATCTCCTGCCAGGTCAGCACCAGCAACCAGGGCCACCAGGGAGAGCCCAGGTACGAGCCTGC
CCAAGTTCACCTGGCGGGAGGGCCAGAAGCAGCTACCGCTCATCGGGTGCGTGCTCCTCCTCATTGCC
CTGGTGGTTTCGCTCATCATCCTCTTCCAGTTCTGCCAGGGCCACACAGGGATCAGGTACAAGGAGCA
GACGGAGAGCTGTCCCAAGCACGCTGTTCGCTGTGACGGGGTGGTGCACTGCAAGCTGAAGAGTGACG
AGCTGGGCTGCGTGAGGTTTGACTGGGACAAGTCTCTGCTTAAAATCTACTCTGGGTCCTCCCATCAG
TGGCTTCCCATCTGTAGCAGCAACTGGAATGACTCCTACTCAGAGAAGACCTGCCAGCAGCTGGGTTT
CGAGAGTGCTCACCGGACAACCGAGGTTGCCCACAGGGATTTTGCCAACAGCTTCTCAATCTTGAGAT
ACAACTCCACCATCCAGGAAAGCCTCCACAGGTCTGAATGCCCTTCCCAGCGGTATATCTCCCTCCAG
TGTTCCCACTGCGGACTGAGGGCCATGACCGGGCGGATCGTGGGAGGGOCGCTGGCCTCCGATAGCAA
GTGGCCTTGGCAAGTGAGTCTGCACTTCGGCACCACCCACATCTGTGGAGGCACGCTCATTGACGCCC
AGTGGGTGCTCACTGCCGCCCACTGCTTCTTCGTCACCCGGGAGAAGGTCCTGGAGGGCTGGAAGGTG
TACGCGGGCACCAGCAACCTGCACCAGTTGCCTGAGGCAGCCTCCATTGCCGAGATCATCATCAACAG
CAATTACACCGATGAGGAGGACGACTATGACATCGCCCTCATGCGGCTGTCCAAGCCCCTGACCCTGT
CCGCTCACATCCACCCTGCTTGCCTCCCCATGCATGGACAGACCTTTAGCCTCAATGAGACCTGCTGG
ATCACAGGCTTTGGCAAGACCAGGGAGACAGATGACAAGACATCCCCCTTCCTCCCGGAGGTGCAGGT
CAATCTCATCGACTTCAACAAATGCAATGACTACTTGGTCTATGACAGTTACCTTACCCCAAGGATGA
TGTGTGCTGGGGACCTTCGTGGGGGCAGAGACTCCTGCCAGGGAGACAGCGGGGGGCCTCTTGTCTGT
GAGCAGAACAACCGCTGGTACCTGGCACGTGTCACCACCTGGGGCACAGGCTGTGGCCAGAGAAACAA
ACCTGGTGTGTACACCAAAGTGACAGAAOTTCTTCCCTGGATTTACAGCAAGATGGAGGTAAGATCCC
TGCAGCAGGACACTGCACCCAGCAGGCTGGGAACTTCCTCAGGTGGGGACCCTGGAGGAGCACCCAGG
NOV12k,
CG50949-06
Protein Sequence SEQ ID NO: 124 586 aa MW at 63152.3 kD
MERDSHGNASPARTPSAGASPAQASPAGTPPCRASPAQASPAQASPAGTPPGRASPAQASPAGTPPGR
ASPGRASPAQASPAQASPARASPALASLSRSSSGRSSSARSASVTTSPTRVYLVRATPVGAVPIRSSP
ARSAPATRATRESPGTSLPKFTWREGQKQLPLIGCVLLLIALVVSLIILFQFWQGHTGIRYKEQRESC
PKHAVRCDGVVDCKLKSDELGCVRFDWDKSLLKIYSGSSHQWLPICSSNWNDSYSEKTCQQLGFESAH
RTTEVAHRDFANSFSILRYNSTIQESLHRSECPSQRYISLQCSHCGLRAMTGRIVGGALASDSKWPWQ
VSLHFGTTHICGGTLIDAQWVLTAAHCFFVTREKVLEGWKVYAGTSNLRQLPEAASIAEIIINSNYTD
EEDDYDIALMRLSKPLTLSAHIHPACLPMHGQTFSLNETCWITGFGKTRETDDKTSPFLREVQVNLID
FKKCNDYLVYDSYLTPRMMCAGDLRGGRDSCQGDSGGPLVCEQNNRWYLAGVTSWGTGCGQRNKPGVY
TKVTEVLPWIYSKMEVRSLQQDTAPSRLGTSSGGDPGGAPRV
NOV12l,
268669017 SEQ ID NO: 125 1218 bp
DNA Sequence ORF Start: at 1 ORF Stop: end of sequence
ACCGGATCCCAGTTCTGGCAGGGCCACACAGGGATCAGGTACAAGGAGCAGAGGGAGAGCTGTCCCA
AGCACGCTGTTCGCTGTGACGGGGTGGTGGACTGCAAGCTGAAGAGTGACGAGCTGGGCTGCGTGAGG
TTTGACTGGGACAAGTCTCTGCTTAAAATCTACTCTGCGTCCTCCCATCAGTGGCTTCCCATCTGTAG
CAGCAACTGGAATGACTCCTACTCAGAGAAGACCTGCCAGCAGCTGGGTTTCGAGAGTGCTCACCGGA
CAACCGAGGTTCCCCACAGGGATTTTGCCAACAGCTTCTCAATCTTGAGATACAACTCCACCATCCAG
GAAAGCCTCCACAGGTCTGAATGCCCTTCCCAGCGGTATATCTCCCTCCAGTGTTCCCACTGCGGACT
GAGGGCCATGACCGGGCGGATCGTGGCAGGGGCGCTGGCCTCGGATAGCAAGTGGCCTTGGCAAGTGA
GTCTGCACTTCGGCACCACCCACATCTGTGGAGGCACGCTCATTGACGCCCAGTGGGTGCTCACTGCC
GCCCACTGCTTCTTCGTGACCCGGGAGAAGGTCCTGGAGGGCTGGAAGGTGTACGCGGGCACCAGCAA
CCTGCACCAGTTGCCTGAGGCAGCCTCCATTGCCGAGATCATCATCAACAGCAATTACACCGATGAGG
AGGACGACTATGACATCGCCCTCATGCGGCTGTCCAAGCCCCTGACCCTGTCCGCTCACATCCACCCT
CCTTGCCTCCCCATGCATGGACAGACCTTTAGCCTCAATGAGACCTGCTGGATCACAGGCTTTGCCAA
GACCAGGGAGACAGATGACAAGACATCCCCCTTCCTCCGGGAGGTGCAGGTCAATCTCATCGACTTCA
AGAAATGCAATGACTACTTGGTCTATGACAGTTACCTTACCCCAAGGATGATGTGTGCTGGGGACCTT
CGTGGGGGCACAGACTCCTGCCAGGGAGACACCGGGGGGCCTCTTGTCTGTGAGCAGAACAACCGCTG
GTACCTGGCAGGTGTCACCACCTGGGGCACAGGCTGTGGCCAGAGAAACAAACCTGGTGTCTACACCA
AAGTGACAGAAGTTCTTCCCTGGATTTACAGCAAGATGGAGGTAAGATCCCTGCAGCACGACACTGCA
CCCAGCAGGCTGGGAACTTCCTCAGGTGGGGACCCTGGAGGAGCACCCAGGGTGCTCGAGGGC
NOV12l,
268669017
Protein Sequence SEQ ID NO: 126 406 aa MW at 45335.5 kD
TGSQFWQGHTGIRYKEQRESCPKHAVRCDGVVDCKLKSDELGCVRFDWDKSLLKIYSGSSHQWLPICS
SNWNDSYSEKTCQQLGFESAHRTTEVAHRDFANSFSILRYNSTIQESLHRSECPSQRYISLQCSHCGL
RAMTGRIVGGALASDSKWPWQVSLHFGTTHICGGTLIDAQWVLTAAHCFFVTREKVLEGWKVYAGTSN
LHQLPEAASIAEIIINSNYTDEEDDYDIALMRLSKPLTLSAHIHPACLPMHGQTFSLNETCWITGFGK
TRETDDKTSPFLREVQVNLIDFKKCNDYLVYDSYLTPRMMCAGDLRGGRDSCQGDSGGPLVCEQNNRW
YLACVTSWGTGCGQRNKPGVYTKVTEVLPWIYSKMEVRSLQQDTAPSRLGTSSGGDPGGAPRVLEG
NOV12m,
CG50949-05 SEQ ID NO: 127 2310 bp
DNA Sequence ORF Start: ATG at 88 ORF Stop: TAA at 1699
CGCCCGGGCAGGTTGAGAAGCCAGGGGCCAAGATGGATCTTCTCCTCGACATCAGCTAAGCCTGGAGG
ACTCTTCCCCTCAGAGACCATGGAGAGGGACAGCCACGGGAATGCATCTCCAGCAAGAACACCTTCAG
CTGGAGCATCTCCAGCCCAGGCATCTCCAGCTGGGACACCTCCAGGCCGGGCATCTCCAGCCCAGGCA
TCTCCAGCCCAGCCATCTCCAGCTGGGACACCTCCGGGCCGGGCATCTCCAGCCCAGGCATCTCCAGC
TGGTACACCTCCAGGCCGGGCATCTCCAGGCCGGGCATCTCCAGCCCAGGCATCTCCAGCCCAGGCAT
CTCCAGCCCAGGCATCTCCAGCCCGGGCATCTCCGGCTCTGGCATCACTTTCCAGGTCCTCATCCGGC
AGGTCATCATCCGCCAGGTCAGCCTCGGTGACAACCTCCCCAACCAGAGTCTACCTTGTTAGAGCAAC
ACCAGTGGGGCCTGTACCCATCCGATCATCTCCTGCCAGGTCAGCACCAGCAACCAGGGCCACCACGG
AGAGCCCAGTCCAGTTCTGGCAGGGCCACACAGGGATCAGGTACAAGGAGCAGAGGGAGACCTGTCCC
AAGCACGCTGTTCGCTGTGACGGGGTGGTGGACTGCAAGCTGAACAGTGACGAGCTGGGCTGCGTGAG
GTTTGACTGGGACAAGTCTCTGCTTAAAATCTACTCTGCGTCCTCCCATCAGTGGCTTCCCATCTGTA
GCAGCAACTGGAATGACTCCTACTCAGAGAAGACCTGCCAGCAGCTGGGTTTCGAGAGTGCTCACCGG
ACAACCGAGGTTGCCCACAGGGATTTTGCCAACAGCTTCTCAATCTTGAGATACAACTCCACCATCCA
GGAAAGCCTCCACAGGTCTGAATGCCCTTCCCAGCGGTATATCTCCCTCCAGTGTTCCCACTGCGCAC
TGAGGGCCATGACCGGGCGGATCGTGCGAGGGGCGCTGGCCTCGGATAGCAAGTGGCCTTGGCAAGTG
AGTCTGCACTTCGGCACCACCCACATCTGTGGAGGCACGCTCATTGACGCCCAGTCGGTGCTCACTGC
CGCCCACTGCTTCTTCGTGACCCGGGAGAAGGTCCTGGAGGGCTGGAAGGTGTACGCGGGCACCAGCA
ACCTGCACCAGTTGCCTGAGGCAGCCTCCATTGCCGAGATCATCATCAACAGCAATTACACCGATGAG
GAGGACGACTATGACATCGCCCTCATGCGGCTGTCCAAGCCCCTGACCCTGTCCGCTCACATCCACCC
TGCTTGCCTCCCCATGCATGGACAGACCTTTAGCCTCAATGAGACCTGCTGGATCACAGGCTTTGGCA
AGACCAGGGAGACAGATGACAAGACATCCCCCTTCCTCCGGGAGGTGCAGGTCAATCTCATCGACTTC
AAGAAATGCAATGACTACTTGGTCTATGACAGTTACCTTACCCCAAGGATGATGTGTGCTGGGGACCT
TCGTGGGGGCAGAGACTCCTGCCAGGGAGACAGCGGGGGGCCTCTTGTCTGTGAGCAGAACAACCGCT
GGTACCTGGCAGGTGTCACCAGCTGGGGCACAGGCTGTGGCCAGAGAAACAAACCTGGTGTGTACACC
AAAGTGACAGAAGTTCTTCCCTGGATTTACAGCAAGATGGAGAGCGAGGTGCGATTCAGAAAATCCTA
ACCAGCTGGCCTGCTGCTCTGCACAGCACCGGCTGCTGTGAAGACTCTGGCCATGGTGACTGGCCATG
TGTGGCATCATCTGGGCTAATGGCCACCGGCCACCATCAGACTCCCACCTCCACTGTCTGCTGCCTCT
GTGTGTGTGTGTGTGTGTGTGTGTGTGCATATGTGTGCATTGCCACTCTCCCAAGTTTTTCAGAAACC
AGCAGAGCTGTCAACTCTTCTCAAAATCCCAGGCTGGAAATTACCTGGAGACAACAGTTGAGTACCGT
GGATGTTCCTACAGGAGTGTCCATAGATGGATGGAGGAGGTGGAGCCCAGAGCCCAAGGAAGAGCTGG
GAATTCTTGCTTCTCTGACCCTCACTTACAGACTAGCCCAGTGTGGGCAGATGCCAGCGGCCCACGTG
GCGCCATTGCTGTCCTGGCATGGATCGTGGGTTTTGGTGGATGCAGCTTCCCAGGGCCTGGACCGTCT
TCGGTGAAAAGCTGCTCCCGTTGGCTTTATGAGCATCAAGTCCTCACCCAGACCCCCTGCTGGTGCCG
TGGATGTCACCAGTCGGACTGTGCTGTGGCTAACCAGGCTGACAACTGAGATGAGGATTCACTGTA
NOV 12m,
CG50949-05
Protein Sequence SEQ ID NO: 128 537 aa MW at 58084.3 kD
MERDSHGNASPARTPSAGASPAQASPAGTPPGRASPAQASPAQASPAGTPPGRASPAQASPAGTPPGR
ASPGRASPAQASPAQASPAQASPARASPALASLSRSSSGRSSSARSASVTTSPTRVYLVRATPVGAVP
IRSSPARSAPATRATRESPVQFWQGHTGIRYKEQRESCPKHAVRCDGVVDCKLKSDELGCVRFDWDKS
LLKIYSGSSUQWLPICSSNWNDSYSEKTCQQLGFESAHRTTEVAHRDFANSFSILRYNSTIQESLHRS
ECPSQRYISLQCSHCGLRAMTGRIVGGALASDSKWPWQVSLHFGTTHICGGTLTDAQWVLTAAHCFFV
TREKVLEGWKVYAGTSNLHQLPEAASIAEIIINSNYTDEEDDYDIALMRLSKPLTLSAHIHPACLPMH
GQTFSLNETCWITGFGKTRETDDKTSPFLREVQVNLIDFKKCNDYLVYDSYLTPRMMCAGDLRGGRDS
CQGDSGGPLVCEQNNRWYLAGVTSWGTGCGQRNKPGVYTKVTEVLPWIYSKMESEVRFRKS
NOV12n,
317431859 SEQ ID NO: 129 1707 bp
DNA Sequence ORF Start: at 1 ORF Stop: end of sequence
ACCGGATCCACCATGGAGAGGGACAGCCACGGGAATGCATCTCCAGCAAGAACACCTTCAGCTGGAG
CATCTCCAGCCCAGGCATCTCCAGCTGGGACACCTCCAGGCCGGGCATCTCCAGCCCAGGCATCTCCA
GCCCAGCCATCTCCAGCTGGGACACCTCCGGGCCGGGCATCTCCAGCCCAGGCATCTCCAGCTGGTAC
ACCTCCAGGCCGGGCATCTCCAGGCCGGGCATCTCCAGCCCAGGCATCTCCAGCCCGGGCATCTCCGG
CTCTGGCATCACTTTCCAGGTCCTCATCCGGCAGGTCATCATCCGCCAGGTCAGCCTCGGTGACAACC
TCCCCAACCAGAGTGTACCTTGTTAGAGCAACACCAGTGGGGGCTGTACCCATCCGATCATCTCCTGC
CAGGTCAGCACCAGCAACCAGGGCCACCAGGGAGAGCCCAGGTACGAGCCTGCCCAAGTTCACCTGGC
GGGAGGGCCAOAAGCAGCTACCGCTCATCGGGTGCGTGCTCCTCCTCATTGCCCTGGTGGTTTCGCTC
ATCATCCTCTTCCAGTTCTGGCAGGCCCACACAGGGATCAGGTACAAGGAGCAGAGGGAGAGCTGTCC
CAAGCACGCTGTTCGCTGTGACGGGGTGGTGGACTGCAAGCTGAAGAGTGACGAGCTGGGCTGCGTGA
GGTTTGACTGGGACAAGTCTCTGCTTAAAATCTACTCTGGGTCCTCCCATCAGTGGCTTCCCATCTGT
AGCAGCAACTGGAATGACTCCTACTCAGAGAAGACCTGCCAGCAGCTGGGTTTCGAGAGTGCTCACCG
GACAACCGAGGTTGCCCACAGGATTTTCCAACAGCTTCTCAATCTTGAGATACAACTCCACCATCC
AGGAAAGCCTCCACAGGTCTGAATGCCCTTCCCAGCGGTATATCTCCCTCCAGTGTTCCCACTGCGGA
CTGAGGGCCATGACCGGGCGGATCGTGGGAGGGGCGCTGGCCTCGGATAGCAAGTGGCCTTGGCAAGT
GAGTCTGCACTTCGGCACCACCCACATCTGTGGAGGCACGCTCATTGACGCCCAGTGGGTGCTCACTG
CCGCCCACTGCTTCTTCGTGACCCGGGAGAAGGTCCTGGAGGGCTGGAAGGTGTACGCCGGCACCAGC
AACCTGCACCAGTTGCCTGAGGCAGCCTCCATTGCCGAGATCATCATCAACAGCAATTACACCGATGA
GGAGGACGACTATGACATCGCCCTCATGCGGCTGTCCAAGCCCCTGACCCTGTCCGCTCACATCCACC
CTGCTTGCCTCCCCATGCATCGACAGACCTTTAGCCTCAATGAGACCTGCTGGATCACAGGCTTTGGC
AAGACCAGGGAGACAGATGACAAGACATCCCCCTTCCTCCGGGAGGTGCAGGTCAATCTCATCGACTT
CAAGAAATGCAATGACTACTTGGTCTATGACAGTTACCTTACCCCAAGGATGATGTGTGCTGGGGACC
TTCGTGGGGGCAGACACTCCTGCCAGGGAGACAGCGGGGGGCCTCTTGTCTGTGAGCAGAACAACCGC
TGGTACCTGGCAGGTGTCACCAGCTGGGGCACAGGCTGTGGCCAGAGAAACAAACCTGGTGTGTACAC
CAAAGTGACAGAAGTTCTTCCCTGCATTTACAGCAAGATGGAGACCGAGGTGCGATTCAGAAAATCCC
TCGAGGGC
NOV12n,
317431859
Protein Sequence SEQ ID NO: 130 569 aa MW at 61684.8 kD
TGSTMERDSHGNASPARTPSAGASPAQASPAGTPPGRASPAQASPAQASPAGTPPGRASPAQASPAGT
PPGRASPGRASPAQASPARASPALASLSRSSSGRSSSARSASVTTSPTRVYLVRATPVGAVPIRSSPA
RSAPATRATRESPGTSLPKFTWREGQKQLPLIGCVLLLIALVVSLIILFQFWQGHTGIRYKEQRESCP
KHAVRCDGVVDCKLKSDELGCVRFDWDKSLLKIYSGSSHQWLPICSSNWNDSYSEKTCQQLGFESAHR
TTEVAHRDFANSFSILRYNSTIQESLHRSECPSQRYISLQCSHCGLRAMTGRIVGGALASDSKWPWQV
SLHFGTTHICGGTLIDAQWVLTAAHCFFVTREKVLEGWKVYAGTSNLHQLPEAASIAEIIINSNYTDE
EDDYDIALMRLSKPLTLSAHIHPACLPMHGQTFSLNETCWITGFGKTRETDDKTSPFLREVQVNLIDF
KKCNDYLVYDSYLTPRMMCAGDLRGGRDSCQGDSGGPLVCEQNNRWYLAGVTSWGTGCGQRNKPGVYT
KVTEXVLPWIYSKMESEVRFRKSLEG
NOV12o,
CG50949-01 SEQ ID NO: 131 1314 bp
DNA Sequence ORF Start: ATG at 1 ORF Stop: TAA at 1264
ATGGAGAGCCCACGTACCAGCCTGCCCAAGTTCACCTGGCGGGAGGGCCAGAAGCAGCTACCGCTCAT
CGGGTGCGTGCTCCTCCTCATTGCCCTGGTGGTTTCGCTCATCATCCTCTTCCAGTTCTGGCAGGGCC
ACACAGGGATCAGGTACAAGGAGCAGAGGGAGAGCTGTCCCAAGCACGCTGTTCGCTGTGACGGGGTG
GTGGACTGCAAGCTGAAGAGTGACGAGCTGGGCTGCGTGAGGTTTCACTGGGACAAGTCTCTGCTTAA
AATCTACTCTGGGTCCTCCCATCAGTGGCTTCCCATCTGTAGCAGCAACTGGAATGACTCCTACTCAG
ACAAGACCTGCCAGCAGCTGGGTTTCGAGAGTGCTCACCGGACAACCGAGGTTGCCCACAGGGATTTT
GCCAACACCTTCTCAATCTTGAGATACAACTCCACCATCCAGGAAAGCCTCCACAGGTCTGAATGCCC
TTCCCAGCGGTATATCTCCCTCCAGTGTTCCCACTGCGGACTGAGGGCCATGACCGGGCGGATCGTGG
GAUGGGCGCTGGCCTCGGATAGCAAGTGGCCTTGCCAAGTCAGTCTGCACTTCGGCACCACCCACATC
TGTGGAGGCACGCTCATTGACGCCCAGTGGGTGCTCACTGCCGCCCACTGCTTCTTCGTGACCCGGGA
GAAGGTCCTGGAGGGCTGGAAGGTGTACGCGGGCACCAGCAACCTGCACCAGTTGCCTGAGGCAGCCT
CCATTGCCGAGATCATCATCAACAGCAATTACACCGATGAGGAGGACGACTATGACATCGCCCTCATG
CGGCTGTCCAAGCCCCTGACCCTGTCCGCTCACATCCACCCTGCTTGCCTCCCCATGCATGGACAGAC
CTTTAGCCTCAATGAGACCTGCTGGATCACAGGCTTTGGCAAGACCAGGGAGACAGATGACAAGACAT
CCCCCTTCCTCCGGGAGGTGCAGGTCAATCTCATCGACTTCAAGAAATGCAATGACTACTTGGTCTAT
GACAGTTACCTTACCCCAAGGATGATGTGTGCTGGGGACCTTCGTGGGGGCAGAGACTCCTGCCAGGG
AGACAGCGGGGGGCCTCTTGTCTGTGAGCAGAACAACCGCTGGTACCTGGCAGGTGTCACCAGCTGGG
GCACAGGCTGTGGCCAGAGAAACAAACCTGGTGTGTACACCAAAGTGACAGAAGTTCTTCCCTCGATT
TACAGCAAGATGGAGAGCGAGGTGCGATTCACAAAATCCTAACCAGCTGGCCTGCTGCTCTGCACAGC
ACCGGCTGCTGTGAAGACTCTG
NOV 12o,
CG50949-01
Protein Sequence SEQ ID NO: 132 421 aa MW at 47484.5 kD
MESPGTSLPKFTWREGQKQLPLIGCVLLLIALVVSLIILFQFWQGHTGIRYKEQRESCPKHAVRCDGV
VDCKLKSDELGCVRFDWDKSLLKIYSGSSHQWLPICSSNWNDSYSEKTCQQLGFESAHRTTEVAHRDF
ANSFSTLRYNSTIQESLHRSECPSQRYISLQCSHCGLRAMTGRIVGGALASDSKWPWQVSLHFGTTHI
CGCTLIDAQWVLTAAHCFFVTREKVLEGWKVYAGTSNLHQLPEAASIAEIIINSNYTDEEDDYDIALM
RLSKPLTLSAHIHPACLPMHGQTFSLNETCWITGFGKTRETDDKTSPFLREVQVNLIDFKKCNDYLVY
DSYLTPRMMCAGDLRGGRDSCQGDSGGPLVCEQNNRWYLAGVTSWGTGCGQRNKPGVYTKVTEVLPWI
YSKMESEVRFTKS
NOV12p,
CG50949-02 SEQ ID NO: 133 1146 bp
DNA Sequence ORF Start: at 1 ORF Stop: end of sequence
TTCCAGTTCTCGCAGGGCCACACAGGGATCAGGTACAAGGAGCAGAGGGAGAGCTGTCCCAAGCACGC
TGTTCGCTGTCACGGGGTGGTGGACTGCAAGCTGAAGAGTGACGAGCTGGGCTCCGTGAGGTTTGACT
GGGACAAGTCTCTGCTTAAAATCTACTCTGGGTCCTCCCATCAGTGGCTTCCCATCTGTAGCAGCAAC
TGGAATGACTCCTACTCAGAGAAGACCTGCCAGCAGCTGGGTTTCGAGAGTGCTCACCGGACAACCGA
GGTTGCCCACAGGGATTTTGCCAACAGCTTCTCAATCTTGAGATACAACTCCACCATCCAGGAAAGCC
TCCACAGGTCTGAATGCCCTTCCCAGCGGTATATCTCCCTCCAGTGTTCCCACTGCGGACTGAGGGCC
ATGACCGGGCGGATCGTGGGAGGGGCGCTGGCCTCGGATAGCAAGTGGCCTTGGCAAGTGAGTCTGCA
CTTCGGCACCACCCACATCTGTGGACGCACGCTCATTGACGCCCAUTGGGTGCTCACTGCCGCCCACT
GCTTCTTCGTGACCCGGGAGAAGGTCCTGGAGCGCTGGAAGGTGTACGCGGGCACCAGCAACCTGCAC
CAGTTGCCTGAGGCAGCCTCCATTGCCGAGATCATCATCAACAGCAATTACACCGATGAGGAGGACGA
CTATGACATCGCCCTCATGCGGCTGTCCAAGCCCCTGACCCTGTCCGCTCACATCCACCCTGCTTGCC
TCCCCATGCATGGACAGACCTTTAGCCTCAATGAGACCTGCTGGATCACAGGCTTTGGCAAGACCAGG
GAGACAGATGACAAGACATCCCCCTTCCTCCGGGAGGTGCAGGTCAATCTCATCGACTTCAAGAAATG
CAATGACTACTTGGTCTATGACAGTTACCTTACCCCAAGGATGATGTGTGCTGGGGACCTTCGTGGGG
GCAGAGACTCCTGCCAGGGAGACAGCGGGGGGCCTCTTGTCTGTGAGCAGAACAACCGCTGGTACCTG
GCAGGTGTCACCAGCTGGGGCACAGGCTGTGGCCAGAGAAACAAACCTGGTGTGTACACCAAAGTGAC
AGAAGTTCTTCCCTGGATTTACAGCAAGATGGAGAGCGACGTGCGATTCACAAAATCC
NOV12p,
CG50949-02
Protein Sequence SEQ ID NO: 134 382 aa MW at 43224.3 kD
FQFWQGHTGIRYKEQRESCPKHAVRCDGVVDCKLKSDELGCVRFDWDKSLLKIYSGSSHQWLPICSSN
WNDSYSEKTCQQLGFESARRTTEVAHRDFANSFSILRYNSTIQESLHRSECPSQRYISLQCSHCGLRA
MTGRIVGGALASDSKWPWQVSLHFGTTHICGGTLIDAQWVLTAAHCFPVTREKVLEGWKVYAGTSNLH
QLPEAASIAEIIINSNYTDEEDDYDIALMRLSKPLTLSAHIHPACLPMHGQTFSLNETCWITGFGKTR
ETDDKTSPFLREVQVNLIDFKKCNDYLVYDSYLTPRMMCAGDLRGGRDSCQGDSGGPLVCEQNNRWYL
AGVTSWGTGCGQRNKPGVYTKVTEVLPWIYSKMESEVRFTKS
NOV12q,
CG50949-04 SEQ ID NO: 135 762 bp
DNA Sequence ORF Start: at 1 ORF Stop: end of sequence
ATCGTGGGAGGGGCGCTGGCCTCGGATAGCAAGTGGCCTTGGCAAGTGAGTCTGCACTTCGGTACCAC
CCACATCTGTGGAGGCACGCTCATTGACGCCCAGTGGGTGCTCACTGCCGCCCACTGCTTCTTCGTGA
CCCGGGAGAAGGTCCTGGAGGGCTGGAAGGTGTACGCGGGCACCAGCAACCTGCACCAGTTGCCTGAG
GCAGCCTCCATTGCCGAGATCATCATCAACAGCAATTACACCGATGAGGAGGACGACTATGACATCGC
CCTCATGCGGCTGTCCAAGCCCCTGACCCTGTCCGCTCACATCCACCCTGCTTGCCTCCCCATGCATG
GACAGACCTTTAGCCTCAATGAGACCTGCTGGATCACAGGCTTTGGCAAGACCAGGGAGACAGATGAC
AAGACATCCCCCTCCCTCCGGGACGTGCAGGTCAATCTCATCGACTTCAAGAAATGCAATGACTACTT
GGTCTATGACAGTTACCTTACCCCAAGGATGATGTGTGCTGGGGACCTTCGTGGGGGCAGAGACTCCT
GCCAGCGAGACAGCGGGGGGCCTCTTGTCTGTGAGCAGAACAACCGCTGGTACCTGGCAGGTGTCACC
AGCTGGGGCACAGGCTGTGGCCAGAGAAACAAACCTGGTGTGTACACCAAAGTGACAGAAGTTCTTCC
CTGGATTTACAGC
NOV12q,
CG50949-04
Protein Sequence SEQ ID NO: 136 231 aa MW at 25643.8 kD
IVGGALASDSKWPWQVSLHFGTTHICGGTLIDAQWVLTAAHCFFVTREKVLEGWKVYAGTSNLHQLPE
AASIAEIIINSNYTDEEDDYDIALMRLSKPLTLSAHIHPACLPMHGQTFSLNETCWITGFGKTRETDD
KTSPSLREVQVNLIDFKKCNDYLVYDSYLTPRMMCAGDLRGGRDSCQGDSGGPLVCEQNNRWYLAGVT
SWGTGCGQRNKPGVYTKVTEVLPWIYS
NOV12r,
CG50949-07 SEQ ID NO: 137 1219 bp
DNA Sequence ORF Start: at 1 ORF Stop: end of sequence
ACCGGATCCCAGTTCTGGCAGGGCCACACAGGGATCAGGTACAAGGAGCAGAGGGAGAGCTGTCCCA
AGCACGCTGTTCGCTGTGACGGGGTGGTGGACTGCAAGCTGAAGAGTGACGAGCTGGGCTGCGTGAGG
TTTGACTGGGACAAGTCTCTGCTTAAAATCTACTCTGGGTCCTCCCATCAGTGGCTTCCCATCTGTAG
CAGCAACTGGAATGACTCCTACTCAGAGAAGACCTGCCAGCAGCTGGGTTTCGAGAGTGCTCACCGGA
CAACCGAGGTTGCCCACAGGGATTTTGCCAACAGCTTCTCAATCTTGAGATACAACTCCACCATCCAG
GAAAGCCTCCACAGGTCTGAATGCCCTTCCCAGCGGTATATCTCCCTCCAGTGTTCCCACTGCGGACT
GAGGGCCATGACCGGGCGGATCGTGGGAGGGGCGCTGGCCTCGGATAGCAAGTGGCCTTGGCAAGTGA
GTCTGCACTTCGGCACCACCCACATCTGTGGAGGCACGCTCATTGACGCCCAGTGGGTGCTCACTGCC
GCCCACTGCTTCTTCGTGACCCGGGAGAAGGTCCTGGAGGGCTGGAAGGTGTACGCGGGCACCAGCAA
CCTGCACCAGTTGCCTGAGGCAGCCTCCATTGCCCAGATCATCATCAACAGCAATTACACCGATGAGG
AGGACGACTATGACATCGCCCTCATGCGGCTGTCCAAGCCCCTGACCCTGTCCGCTCACATCCACCCT
GCTTGCCTCCCCATGCATGGACAGACCTTTAGCCTCAATGAGACCTGCTGGATCACAGGCTTTGGCAA
GACCAGGGAGACAGATGACAAGACATCCCCCTTCCTCCGGGAGGTGCAGGTCAATCTCATCGACTTCA
AGAAATGCAATGACTACTTGGTCTATGACAGTTACCTTACCCCAAGGATGATGTGTGCTGGGGACCTT
CGTGGGGGCAGAGACTCCTGCCAGGGAGACAGCGGGGCGCCTCTTGTCTCTGAGCAGAACAACCGCTG
GTACCTGGCAGGTGTCACCAGCTGGGGCACAGGCTGTGGCCAGAGAAACAAACCTGGTGTGTACACCA
AAGTGACAGAAGTTCTTCCCTGGATTTACAGCAAGATGGAGGTAAGATCCCTGCAGCAGGACACTGCA
CCCAGCAGGCTGGGAACTTCCTCAGGTGGGGACCCTGGAGGAGCACCCAGGGTGCTCGAGGGC
NOV 12r,
CG50949-07
Protein Sequence SEQ ID NO: 138 406 aa MW at 45335.5 kD
TGSQFWQGHTGIRYKEQRESCPKHAVRCDGVVDCKLKSDELGCVRFDWDKSLLKIYSGSSHQWLPICS
SNWNDSYSEKTCQQLGFESAHRTTEVAHRDFANSFSILRYNSTIQESLHRSECPSQRYISLQCSHCGL
RAMTGRIVGGALASDSKWPWQVSLHFGTTHICGGTLIDAQWVLTAAHCFFVTREKVLEGWKVYAGTSN
LHQLPEAASIAEIIINSNYTDEEDDYDIALMRLSKPLTLSAHIHPACLPMHGQTFSLNETCWITGFGK
TRETDDKTSPFLREVQVNLIDFKKCNDYLVYDSYLTPRMMCAGDLRGGRDSCQGDSGGPLVCEQNNRW
YLAGVTSWGTGCGQRNKPGVYTKVTEVLPWIYSKMEVRSLQQDTAPSRLGTSSGGDPGGAPRVLEG
NOV12s,
13374729 SNP SEQ ID NO: 139 2432 bp SNP:
for CG50949-03 ORF Start: ATG position 902 A/C
DNA Sequence at 112 ORF Stop: TAG at 1870
GGACACTGACATGGACTGAAGGAGTAGAAAACATGCCTGAGAAGCCAGGGGCCAAGATGGATCTTCTCCT
CGACATCAGCTAAGCCTGGAGGACTCTCCCCCTCAGAGACCATGGAGAGGGACAGCCACGGGAATGCATC
TCCAGCAAGAACACCTTCAGCTGGAGCATCTCCAGCCCAGGCATCTCCAGCTGGGACACCTCCAGGCCGG
GCATCTCCAGCCCAGGCATCTCCAGCCCAGGCATCTCCAGCTGGGACACCTCCGCGCCGGGCATCTCCAG
CCCAGGCATCTCCAGCTGGTACACCTCCAGGCCGGGCATCTCCAGGCCGGGCATCTCCAGCCCAGGCATC
TCCAGCCCAGGCATCTCCACCCCGGGCATCTCCGGCTCTGGCATCACTTTCCAGGTCCTCATCCGGCAGG
TCATCATCCGCCAGGTCGGCCTCGGTGACAACCTCCCCAACCAGAGTGTACCTTGTTAGAGCAACACCAG
TGGGGGCTGTACCCATCCGATCATCTCCTGCCAGGTCAGCACCAGCAACCAGGGCCACCAGGGAGAGCCC
AGGTACGAGCCTGCCCAAGTTCACCTGGCGGGAGGGCCAGAAGCAGCTACCGCTCATCGGGTGCGTGCTC
CTCCTCATTGCCCTGGTGGTTTCGCTCATCATCCTCTTCCAGTTCTGGCAGGGCCACACAGGGATCAGGT
ACAAGGAGCAGAGGGAGAGCTGTCCCAAGCACGCTGTTCGCTGTGACGGGGTGGTGGACTGCAAGCTGAA
GAGTGACGAGCTGGGCTGCGTGAGGTTTGACTGGGACAAGTCTCTGCTTAAAATCTACTCTGGGTCCTCC
CATCAGTGGCTTCCCATCTGTAGCAGCAACTGGAATCACTCCTACTCAGAGAAGACCTGCCCGCAGCTGG
GTTTCGAGAGTGCTCACCGGACAACCGAGGTTGCCCACAGGGATTTTGCCAACAGCTTCTCAATCTTGAG
ATACAACTCCACCATCCAGGAAAGCCTCCACAGGTCTGAATGCCCTTCCCAGCGGTATATCTCCCTCCAG
TGTTCCCACTGCGGACTGAGGGCCATGACCGGGCGGATCGTGGGAGGGGCGCTGGCCTCGGATAGCAAGT
GGCCTTGGCAAGTGAGTCTGCACTTCGGCACCACCCACATCTGTGGAGGCACGCTCATTGACGCCCAGTG
GGTGCTCACTGCCGCCCACTGCTTCTTCGTGACCCGGGAGAAGGTCCTGGAGGGCTGGAGGGTGTACGCG
GGCACCAGCAACCTGCACCAGTTGCCTGAGGCAGCCTCCATTGCCGAGATCATCATCAACAGCAATTACA
CCGATGAGGAGGACGACTATGACATCGCCCTCATGCGGCTGTCCAAGCCCCTGACCCTGTCCGCTCACAT
CCACCCTGCTTGCCTCCCCATGCATGGACAGACCTTTAGCCTCAATGAGACCTGCTGGATCACAGGCTTT
GGCAAGACCAGGCAGACACATGACAAGACATCCCCCTTCCTCCGGGAGGTGCAGGTCAATCTCATCGACT
TCAAGAAATGCAATGACTACTTGGTCTATGACAGTTACCTTACCCCAAGGATGATGTGTGCTGGGGACCT
TCGTGGGGGCAGAGACTCCTGCCACGGAGACAGCGGGGGGCCTCTTGTCTGTGAGCAGAACAACCGCTGG
TACCTGGCAGGTGTCACCAGCTGGGGCACAGGCTGTCGCCAGAGAAACAAACCTGGTGTGTACACCAAAG
TGACAGAAGTTCTTCCCTGGATTTACAGCAAGATGGAGGTAAGATCCCTGCAGCAGGACACTGCACCCAG
CAGGCTGGGAACTTCATCAGGTGGGGACCCTGGAGGAGCACCCAGGGTGTAGGCAGAGGTCCCCTCAGCG
TCCCCATATTCGGGGGGTGTTCTGGACAGGGTCAAATGTGATGCCTGGGGTCAATCCCAGCTGTCTGTGT
TTCTTTCCCTGCTTTTCTTCCCTCAGAACAGAGCTCAGCGGGTTGAAAAAGGGTGGACCTACAGGCCAGG
CAGGCAGTTGCTGGGCAGATGTTCTCCCAGAAGTATTTTTTTGTGTAAGGTTGCAATGGACTTTGAAAAC
GTTTCAGTTTCTGCAGAGGATTTTGTGATAGTCTTTGTTATCAAGCATTTATGCATGGGAATCCGCTCTT
CATGGCCTTTCCCAGCTCTGTTTGTTTTAGTCTTTTTGATTTTCTTTTTGTTGTTGTTGTTGTCTTTTTT
TAAAAACACAAGTGACTCCATTTTAACTCTGACAACTTTCACAGCTGTCACCAGAATGCTCCCTGAGAAC
TACCATTCTTTCCCTTTCCCACTTAAAATATTTCATCAGAACCTCACCACTATCATAAAAGAGTATAAAG
TAATAAAATAATAAAAAGCGAAAAAAAAAAAAAAAAAAAAAAAAAAAAAAA
NOV 12s,
13374729 SNP
for CG50949-03 SEQ ID NO: 140 586 aa SNP: Gln to Pro
Protein Sequence at position 264
MERDSHGNASPARTPSAGASPAQASPAGTPPGRASPAQASPAQASPAGTPPGRASPAQASPAGTPPGRAS
PGRASPAQASPAQASPARASPALASLSRSSSGRSSSARSASVTTSPTRVYLVPATPVGAVPIRSSPARSA
PATRATRESPGTSLPKFTWREGQKQLPLIGCVLLLIALVVSLILLFQFWQGHTGIRYKEQRESCPKHAVR
CDGVVDCKLKSDELGCVRFDWDKSLLKIYSGSSHQWLPICSSNWNDSYSEKTCPQLGFESAHRTTEVAHR
DFANSFSILRYNSTIQESLHRSECPSQRYISLQCSHCGLRAMTGRIVGGALASDSKWPWQVSLHFGTTHI
CGGTLIDAQWVLTAAHCFFVTREKVLEGWKVYAGTSNLHQLPEAASIAEIIINSNYTDEEDDYDIALMRL
SKPLTLSAHIHPACLPMHGQTFSLNETCWITGFGKTRETDDKTSPFLREVQVNLIDFKKCNDYLVYDSYL
TPRMMCAGDLRGGRDSCQGDSGGPLVCEQNNRWYLAGVTSWGTGCGQRNKPGVYTKVTEVLPWIYSKMEV
RSLQQDTAPSRLGTSSGGDPGGAPRV
NOV12t,
13374730 SNP SEQ ID NO: 141
for CG50949-03 ORF Start: ATG at 2432 bp, SNP: 1313 T/C
DNA Sequence 112 ORF Stop: TAG at 1870
CGACACTGACATGGACTGAAGGAGTAGAAAACATGCCTGAGAAGCCAGGGGCCAAGATGGATCTTCTCCT
CGACATCAGCTAAGCCTGGAGGACTCTCCCCCTCAGAGACCATGGAGAGGGACAGCCACGGGAATGCATC
TCCAGCAAGAACACCTTCAGCTGGAGCATCTCCAGCCCAGGCATCTCCAGCTGGGACACCTCCACGCCGG
GCATCTCCAGCCCAGGCATCTCCAGCCCAGGCATCTCCAGCTGGGACACCTCCGGGCCGGGCATCTCCAC
CCCAGGCATCTCCAGCTGGTACACCTCCAGGCCGGGCATCTCCAGGCCGGGCATCTCCAGCCCAGGCATC
TCCAGCCCAGGCATCTCCAGCCCGGGCATCTCCGGCTCTGGCATCACTTTCCAGGTCCTCATCCGGCACG
TCATCATCCGCCAGGTCGGCCTCGGTGACAACCTCCCCAACCAGAGTGTACCTTGTTAGAGCAACACCAG
TGGGGGCTGTACCCATCCGATCATCTCCTGCCAGGTCAGCACCAGCAACCAGCGCCACCAGGGACAGCCC
AGGTACGAGCCTGCCCAAGTTCACCTGGCCGGAGGCCCAGAAGCAGCTACCGCTCATCGGGTGCGTGCTC
CTCCTCATTGCCCTGGTGGTTTCGCTCATCATCCTCTTCCAGTTCTGGCAGGGCCACACAGGGATCAGGT
ACAAGGAGCAGAGGGAGAGCTGTCCCAAGCACGCTGTTCGCTGTGACGGGGTGGTCGACTGCAAGCTGAA
GAGTGACGAGCTCGGCTGCGTGAGGTTTGACTGGGACAAGTCTCTGCTTAAAATCTACTCTCGGTCCTCC
CATCAGTGGCTTCCCATCTGTAGCAGCAACTGGAATGACTCCTACTCAGAGAAGACCTGCCAGCAGCTGG
GTTTCGAGAGTGCTCACCGGACAACCGAGGTTGCCCACAGGGATTTTGCCAACAGCTTCTCAATCTTGAG
ATACAACTCCACCATCCAGGAAAGCCTCCACAGGTCTGAATGCCCTTCCCAGCGGTATATCTCCCTCCAG
TGTTCCCACTGCGGACTGAGGGCCATGACCGGGCGGATCGTGGGAGGGGCGCTGGCCTCGGATAGCAAGT
GGCCTTGGCAAGTGAGTCTGCACTTCGGCACCACCCACATCTGTGGAGGCACGCTCATTGACGCCCAGTG
GGTGCTCACTGCCGCCCACTGCTTCTTCGTGACCCGGGAGAAGGTCCTGGAGGGCTGGAAGGTGTACGCG
GGCACCAGCAACCTGCACCAGTTGCCTGAGGCAGCCTCCATTGCCGACATCACCATCAACAGCAATTACA
CCGATGAGGAGGACGACTATGACATCGCCCTCATGCGGCTGTCCAAGCCCCTGACCCTGTCCGCTCACAT
CCACCCTGCTTGCCTCCCCATGCATGGACACACCTTTAGCCTCAATGAGACCTGCTGGATCACAGGCTTT
GCCAAGACCAGGGAGACAGATGACAAGACATCCCCCTTCCTCCGGGAUGTGCAGGTCAATCTCATCGACT
TCAAGAAATGCAATGACTACTTGGTCTATGACAGTTACCTTACCCCAAGGATCATGTGTGCTGGGGACCT
TCGTGCGGGCAGAOACTCCTGCCAGGGAGACAGCGGGGGGCCTCTTGTCTGTGAGCAGAACAACCGCTGG
TACCTGGCAGGTGTCACCAGCTGGGGCACAGGCTGTGGCCAGAGAAACAAACCTGGTGTGTACACCAAAG
TGACAGAAGTTCTTCCCTGGATTTACAGCAAGATGGAGGTAAGATCCCTGCAGCAGGACACTGCACCCAG
CAGGCTGGGAACTTCCTCAGGTGGGGACCCTGOAGGACCACCCAGGGTGTAGGCAGAGGTCCCCTCAGCG
TCCCCATATTCGGCGGGTGTTCTGGACAGGGTCAAATGTGATGCCTGGGGTCAATCCCAGCTGTCTGTGT
TTCTTTCCCTGCTTTTCTTCCCTCAGAACAGAGCTCAGCGGGTTGAAAAAGGGTGGACCTACAGGCCACG
CAGGCAGTTGCTGGGCAGATGTTCTCCCAGAAGTATTTTTTTGTGTAAGGTTGCAATCGACTTTGAAAAC
GTTTCAGTTTCTGCAGAGGATTTTGTGATAGTCTTTGTTATCAAGCATTTATGCATGGGAATCCGCTCTT
CATGGCCTTTCCCAGCTCTGTTTGTTTTAGTCTTTTTGATTTTCTTTTTGTTGTTGTTGTTGTCTTTTTT
TAAAAACACAAGTGACTCCATTTTAACTCTGACAACTTTCACAGCTGTCACCAGAATGCTCCCTGAGAAC
TACCATTCTTTCCCTTTCCCACTTAAAATATTTCATCAGAACCTCACCACTATCATAAAAGAGTATAAAG
TAATAAAATAATAAAAAGCGAAAAAAAAAAAAAAAAAAAAAAAAAAAAAAAA
NOV12t,
13374730 SNP
for CG50949-03 SEQ ID NO: 142 586 aa SNP: Ile to Thr
Protein Sequence at position 401
MERDSHGNASPARTPSAGASPAQASPAGTPPGRASPAQASPAQASPAGTPPGRASPAQASPAGTPPCRAS
PGRASPAQASPAQASPAPASPALASLSRSSSGRSSSARSASVTTSPTRVYLVRATPVGAVPIRSSPARSA
PATRATRESPGTSLPKFTWREGQKQLPLIGCVLLLIALVVSLIILFQFWQGHTGIRYKEQRESCPKHAVR
CDGVVDCKLKSDELGCVRFDWDKSLLKIYSGSSHQWLPICSSNWNDSYSEKTCQQLGFESAHRTTEVAHR
DFANSFSILRYNSTIQESLHRSECPSQRYISLQCSHCGLRAMTGRIVGGALASDSKWPWQVSLHFGTTHI
CGGTLIDAQWVLTAAHCFFVTREKVLEGWKVYAGTSNLhQLPEAASIAEITINSNYTDEEDDYDIALMRL
SKPLTLSAHIHPACLPMHGQTFSLNETCWITGFGKTRETDDKTSPFLREVQVNLTDFKKCHDYLVYDSYL
TPRMMCAGDLRGGRDSCQGDSGGPLVCEQNNRWYLAGVTSWGTGCGQRNKPGVYTKVTEVLPWIYSKMEV
RSLQQDTAPSRLGTSSGGDPGGAPRV
NOV12u,
13374731 SNP
for CG50949-03 SEQ ID NO: 143 2432 bp SNP: 1350 T/G
DNA Sequence ORF Start: ATG at 112 ORF Stop: TAG at 1870
GGACACTGACATGGACTGAAGGAGTAGAAAACATGCCTGAGAAGCCAGGGGCCAAGATGGATCTTCTCCT
CGACATCAGCTAAGCCTGGAGGACTCTCCCCCTCAGAGACCATGGAGAGGGACAGCCACGGGAATGCATC
TCCAGCAAGAACACCTTCAGCTGGAGCATCTCCAGCCCAGGCATCTCCAGCTGGGACACCTCCAGGCCGG
GCATCTCCAGCCCAGGCATCTCCAGCCCAGGCATCTCCAGCTGGGACACCTCCGGGCCGGGCATCTCCAG
CCCAGGCATCTCCAGCTGGTACACCTCCAGGCCGGCCATCTCCAGGCCGGGCATCTCCAGCCCACGCATC
TCCAGCCCAGGCATCTCCAGCCCGGGCATCTCCGGCTCTGGCATCACTTTCCAGGTCCTCATCCGGCAGG
TCATCATCCGCCAGGTCGGCCTCGGTGACAACCTCCCCAACCAGAGTGTACCTTGTTAGAGCAACACCAG
TGGGGGCTGTACCCATCCGATCATCTCCTGCCAGGTCAGCACCAGCAACCAGGGCCACCAGGGAGAGCCC
AGGTACGAGCCTGCCCAAGTTCACCTGGCGGGAGGGCCAGAAGCAGCTACCGCTCATCGGGTGCGTGCTC
CTCCTCATTGCCCTGGTGGTTTCGCTCATCATCCTCTTCCAGTTCTOGCAGGGCCACACAGGGATCAGGT
ACAAGGAGCAGAGGGAGAGCTGTCCCAAOCACGCTGTTCGCTGTGACGGGGTGGTGGACTGCAAGCTGAA
GAGTGACGAGCTGGGCTGCGTGAGGTTTGACTGGGACAAGTCTCTGCTTAAAATCTACTCTGGGTCCTCC
CATCAGTGGCTTCCCATCTGTAGCAGCAACTGGAATGACTCCTACTCAGAGAAGACCTGCCAGCAGCTGG
GTTTCGAGAGTGCTCACCGGACAACCGAGGTTGCCCACAGGGATTTTGCCAACAGCTTCTCAATCTTGAG
ATACAACTCCACCATCCAGGAAAGCCTCCACAGGTCTGAATGCCCTTCCCAGCGGTATATCTCCCTCCAG
TGTTCCCACTGCGGACTGAGGGCCATGACCGGGCGGATCGTCGGAGGGGCGCTGGCCTCGGATAGCAAGT
GGCCTTGGCAAGTGAGTCTGCACTTCGGCACCACCCACATCTGTGGAGGCACGCTCATTGACGCCCAGTG
GGTGCTCACTGCCGCCCACTGCTTCTTCGTGACCCGGGAGAAGGTCCTGGAGGGCTGGAAGGTGTACGCG
GGCACCAGCAACCTGCACCAGTTGCCTGAGGCAGCCTCCATTGCCGAGATCATCATCAACAGCAATTACA
CCGATGAGGAGGACGACTAGGACATCGCCCTCATGCGOCTGTCCAAGCCCCTGACCCTGTCCGCTCACAT
CCACCCTGCTTCCCTCCCCATGCATGGACAGACCTTTAGCCTCAATGAGACCTGCTGGATCACAGGCTTT
GGCAAGACCAGGGAGACAGATGACAAGACATCCCCCTTCCTCCGGGAGGTGCAGGTCAATCTCATCGACT
TCAAGAAATGCAATGACTACTTGGTCTATGACAGTTACCTTACCCCAAGGATGATGTGTGCTGGGGACCT
TCGTGCGGGCAGAGACTCCTGCCAGGGAGACAGCGGGGGGCCTCTTGTCTGTGAGCAGAACAACCGCTGG
TACCTGGCAGGTGTCACCAGCTGGGGCACAGCCTGTGGCCAGAGAAACAAACCTGGTGTGTACACCAAAG
TGACAGAAGTTCTTCCCTGGATTTACAGCAAGATGGAGGTAAGATCCCTGCAGCAGGACACTGCACCCAG
CAGGCTGGGAACTTCCTCAGGTGGGGACCCTGGAGGAGCACCCAGGGTGTAGGCAGAGGTCCCCTCAGCG
TCCCCATATTCGGGGGGTGTTCTGGACAGGGTCAAATGTGATGCCTGGGGTCAATCCCAGCTGTCTGTGT
TTCTTTCCCTGCTTTTCTTCCCTCAGAACAGAGCTCAGCGGGTTGAAAAAGGGTGGACCTACAGGCCAGG
CAGGCAGTTGCTGGGCAGATGTTCTCCCAGAAGTATTTTTTTGTGTAAGG
TTGCAATGGACTTTGAAAACGTTTCAGTTTCTGCAGAGGATTTTGTGATAGTCTTTGTTATCAAGCATTT
ATGCATGGGAATCCGCTCTTCATGGCCTTTCCCAGCTCTGTTTGTTTTAGTCTTTTTGATTTTCTTTTTG
TTGTTGTTGTTGTCTTTTTTTAAAAACACAAGTGACTCCATTTTAACTCTGACAACTTTCACAGCTGTCA
CCAGAATGCTCCCTGAGAACTACCATTCTTTCCCTTTCCCACTTAAAATATTTCATCAGAACCTCACCAC
TATCATAAAAGAGTATAAAGTAATAAAATAATAAAAAGCGAAAAAAAAAAAAAAAAAAAAAAAAAAAAAA
AA
NOV12u,
13374731 SNP
for CG50949-03 SEQ ID NO: 144 586 aa SNP: Tyr to STOP
Protein Sequence at position 413
MERDSHGNASPARTPSAGASPAQASPAGTPPGRASPAQASPAQASPAGTPPGRASPAQASPAGTPPGRAS
PGRASPAQASPAQASPARASPALASLSRSSSGRSSSARSASVTTSPTRVYLVRATPVGAVPIRSSPARSA
PATRATRESPGTSLPKFTWREGQKQLPLIGCVLLLIALVVSLIILFQFWQGHTGIRYKEQRESCPKHAVR
CDGVVDCKLKSDELGCVRFDWDKSLLKIYSGSSHQWLPICSSNWNDSYSEKTCQQLGFESAHRTTEVAHR
DFANSFSTLRYNSTIQESLHRSECPSQRYISLQCSHCGLRAMTGRIVGGALASDSKWPWQVSLHFGTTHI
CGGTLIDAQWVLTAAHCFFVTREKVLEGWKVYAGTSWLHQLPEAASIAEIIINSNYTDEEDD*

A ClustalW comparison of the above protein sequences yields the following sequence alignment shown in Table 12B.

TABLE 12B
Comparison of the NOV12 protein sequences.

| | |
|---|---|
| NOV12a | ----MERDSHGNASPARTPSAGASPAQASPAGTPPGRASPAQASPAQASPAGTPPGRASP |
| NOV12b | ------------------------------------------------------------ |
| NOV12c | ------------------------------------------------------------ |
| NOV12d | ------------------------------------------------------------ |
| NOV12e | ------------------------------------------------------------ |
| NOV12f | ------------------------------------------------------------ |
| NOV12g | ------------------------------------------------------------ |
| NOV12h | ------------------------------------------------------------ |
| NOV12i | TGSTMERDSHGNASPARTPSAGASPAQASPAGTPPGRASPAQASPAQASPAGTPPGRASP |
| NOV12j | TGSTMERDSHGNASPARTPSAGASPAQASPAOTPPGRASPAQASPAQASPAGTPPGRASP |
| NOV12k | ----MERDSHGNASPARTPSAGASPAQASPAGTPPGRASPAQASPAQASPAGTPPGRASP |
| NOV12l | ------------------------------------------------------------ |
| NOV12m | ----MERDSHGNASPARTPSAGASPAQASPAGTPPGRASPAQASPAQASPAGTPPGRASP |
| NOV12n | TGSTMERDSHGNASPARTPSAGASPAQASPAGTPPGRASPAQASPAQASPAGTPPGRASP |
| NOV12o | ------------------------------------------------------------ |
| NOV12p | ------------------------------------------------------------ |
| NOV12q | ------------------------------------------------------------ |
| NOV12r | ------------------------------------------------------------ |
| NOV12a | AQASPAGTPPGRASPGRASPAQASPAQAS-----PARASPALASLSRSSSGRSSSARSAS |
| NOV12b | ------------------------------------------------------------ |
| NOV12c | ------------------------------------------------------------ |
| NOV12d | ------------------------------------------------------------ |
| NOV12e | ------------------------------------------------------------ |
| NOV12f | ------------------------------------------------------------ |
| NOV12g | ------------------------------------------------------------ |
| NOV12h | ------------------------------------------------------------ |
| NOV12i | AQASPAGTPPGRASPGRASPAQASPAQAS-----PARASPALASLSRSSSGRSSSARSAS |
| NOV12j | AQASPAGTPPGRASP-------------------------ALASLSRSSSGRSSSARSAS |
| NOV12k | AQASPAGTPPGRASPGRASPAQASPAQAS-----PARASPALASLSRSSSGRSSSARSAS |
| NOV12l | ------------------------------------------------------------ |
| NOV12m | AQASPAGTPPGRASPGRASPAQASPAQASPAQASPARASPALASLSRSSSGRSSSARSAS |
| NOV12n | AQASPAGTPPGRASPGRASPAQAS----------PARASPALASLSRSSSGRSSSARSAS |
| NOV12o | ------------------------------------------------------------ |
| NOV12p | ------------------------------------------------------------ |
| NOV12q | ------------------------------------------------------------ |
| NOV12r | ------------------------------------------------------------ |
| NOV12a | VTTSPTRVYLVRATPVGAVPIRSSPARSAPATRATRESPGTSLPKFTWREGQKQLPLIGC |
| NOV12b | ------------------------------------------------------------ |
| NOV12c | ------------------------------------------------------------ |
| NOV12d | ------------------------------------------------------------ |
| NOV12e | ------------------------------------------------------------ |
| NOV12f | ------------------------------------------------------------ |
| NOV12g | ------------------------------------------------------------ |
| NOV12h | ------------------------------------------------------------ |
| NOV12i | VTTSPTRVYLVRATPVGAVPIRSSPARSAPATRATRESPGTSLPKFTWREGQKQLPLIGC |
| NOV12j | VTTSFTRVYLVRATPVGAVFIRSSPARSAPATRATRESPGTSLPKFTWREGQKQLFLIGC |
| NOV12k | VTTSPTRVYLVRATPVGAVPIRSSPARSAPATRATRESPGTSLPKFTWREGQKQLPLIGC |
| NOV12l | ------------------------------------------------------------ |
| NOV12m | VTTSPTRVYLVRATPVGAVPIRSSPARSAPATRATRESP--------------------- |
| NOV12n | VTTSFTRVYLVRATPVGAVPIRSSPARSAPATRATRESPGTSLPKFTWREGQKQLPLIGC |
| NOV12o | -----------------------------------MESPGTSLPKFTWREGQKQLPLIGC |
| NOV12p | ------------------------------------------------------------ |
| NOV12q | ------------------------------------------------------------ |
| NOV12r | ------------------------------------------------------------ |
| NOV12a | VLLLIALVVSLIILFQFWQGHTGIRYKEQRESCPKHAVRCDGVVDCKLKSDELGCVRFDW |
| NOV12b | ------------------------------------------------------------ |
| NOV12c | ------------------------------------------------------------ |
| NOV12d | -------------------GSTGIRYKEQRESCPKHAVRCDGVVDCKLKSDELGCVRFDW |
| NOV12e | -----------------TGSHTGIRYKEQRESCPKHAVRCDGVVDCKLKSDELGCVRFDW |
| NOV12f | -----------------TGSHTGIRYKEQRESCPKHAVRCDGVVDCKLKSDELGCVRFDW |
| NOV12g | -----------------TGSHTGIRYKEQRESCPKHAVRCDGVVDCKLKSDELGCVRFDW |
| NOV12h | -----------------TGSHTGIRYKEQRESCPKHAVRCDGVVDCKLKSDELGCVRFDW |
| NOV12i | VLLLIALVVSLIILFQFWQGHTGIRYKEQRESCPKHAVRCDGVVDCKLKSDELGCVRFDW |
| NOV12j | VLLLIALVVSLIILFQFWQGHTGIRYKEQRESCPKHAVRCDGVVDCKLKSDELGCVRFDW |
| NOV12k | VLLLIALVVSLIILFQFWQGHTGIRYKEQRESCPKHAVRCDGVVDCKLKSDELGCVRFDW |
| NOV12l | ------------TGSQFWQGHTGIRYKEQRESCPKHAVRCDGVVDCKLKSDELCCVRFDW |
| NOV12m | --------------VQFWQGHTGIRYKEQRESCPKHAVRCDGVVDCKLKSDELGCVRFDW |
| NOV12n | VLLLIALVVSLIILFQFWQGHTGIRYKEQRESCPKHAVRCDGVVDCKLKSDELGCVRFDW |
| NOV12o | VLLLIALVVSLIILFQFWQGHTGIRYKEQRESCPKHAVRCDGVVDCKLKSDELGCVRFDW |
| NOV12p | --------------FQFWQGHTGIRYKEQRESCPKHAVRCDGVVDCKLKSDELGCVRFDW |
| NOV12q | ------------------------------------------------------------ |
| NOV12r | -------------TGSQFWQGHTGIRYKEQRESCPHAVRCDGVVDCKLKSDELGCVRFDW |
| NOV12a | DKSLLKIYSGSSHQWLPICSSNWNDSYSEKTCQQLGFESAHRTTEVAHRDFANSFSILRY |
| NOV12b | ------------------------------------------------------------ |
| NOV12c | ------------------------------------------------------------ |
| NOV12d | DKSLLKIYSGSSHQWLPICSSNWNDSYSEKTCQQLGFE---------------------- |
| NOV12e | DKSLLKIYSGSSHQWLPICSSNWNDSYSEKTCQQLGFESAHRTTEVAHRDFANSFSILRY |
| NOV12f | DKSLLKIYSGSSHQWLPICSSNWNDSYSEKTCQQLGFE---------------------- |
| NOV12g | DKSLLKIYSGSSHQWLPICSSNWNDSYSEKTCQQLGFESAHRTTEVAHRDFANSFSILRY |
| NOV12h | DKSLLKIYSGSSHQWLPICSSNWNDSYSEKTCQQLGFESAHRTTEVAHRDFANSFSILRY |
| NOV12i | DKSLLKIYSGSSHQWLPICSSNWNDSYSEKTCQQLGFESAHRTTEVAHRDFANSFSILRY |
| NOV12j | DKSLLKIYSGSSHQWLPICSSNWNDSYSEKTCQQLGFE---------------------- |
| NOV12k | DKSLLKIYSGSSHQWLPICSSNWNDSYSEKTCQQLGFESAHRTTEVAHRDFANSFSILRY |
| NOV12l | DKSLLKIYSGSSHQWLPICSSNWNDSYSEKTCQQLGFESAHRTTEVAHRDFANSFSILRY |
| NOV12m | DKSLLKIYSGSSHQWLPICSSNWNDSYSEKTCQQLGFESAHRTTEVAHRDFANSFSILRY |
| NOV12n | DKSLLKIYSGSSHQWLPICSSNWNDSYSEKTCQQLGFESAHRTTEVAHRDFANSFSILRY |
| NOV12o | DKSLLKIYSGSSHQWLPICSSNWNDSYSEKTCQQLGFESAHRTTEVAHRDFANSFSILRY |
| NOV12p | DKSLLKIYSGSSHQWLPICSSNWNDSYSEKTCQQLGFESAHRTTEVAHRDFANSFSILRY |
| NOV12q | ------------------------------------------------------------ |
| NOV12r | DKSLLKIYSGSSHQWLPICSSNWNDSYSEKTCQQLGFESAHRTTEVAHRDFANSFSILRY |
| NOV12a | NSTIQESLHRSECPSQRYISLQCSHCGLRAMTGRIVGGALASDSKWPWQVSLHFGTTHIC |
| NOV12b | ----------------------------SLWIGSIVGGALASDSKWPWQVSLHFGTTHIC |
| NOV12c | ----------------------------SLWIGSIVGGALASDSKWPWQVSLNFGTTHIC |
| NQV12d | ---------RSECPSQRYISLQCSHCGLRANTGRIVGGALASDSKWPWQVSLHFGTTHIC |
| NOV12e | NSTIQESLHRSECPSQRYISLQCSHCGLRANTGRIVGGALASDSKWPWQVSLHFGTTHIC |
| NOV12f | ---------RSECPSQRYISLQCSHCGLRAMTGRIVGGALASDSKWPWQVSLHFGTTHIC |
| NOV12g | NSTIQESLHRSECPFQRYISLQCSHCGLRAMTGRIVGGALVSDSKWPWQVSLHFGTTHIC |
| NOV12h | NSTTQESLHRSECPSQRYISLQCSHCGLRAMTGRIVGGALASDSKWPWQVSLHFGTTHIC |
| NOV12i | NSTIQESLHRSECPSQRYISLQCSHCGLRAMTGRIVGGALASDSKWPWQVSLHFGTTHIC |
| NOV12j | ---------RSECPSQRYISLQCSHCGLRAMTGRIVGGALASDSKWPWQVSLHFGTTHIC |
| NOV12k | NSTIQESLHRSECPSQRYISLQCSHCGLRAMTGRIVGCALASDSKWPWQVSLHFGTTHIC |
| NOV12l | NSTIQESLHRSECPSQRYISLQCSHCGLRAMTGRIVGGALASDSKWPWQVSLHFGTTHIC |
| NOV12m | NSTIQESLHRSECPSQRYISLQCSHCGLRAMTGRIVGGALASDSKWPWQVSLHFGTTHIC |
| NOV12n | NSTIQESLHRSECPSQRYISLQCSHCGLRAMTGRIVGGALASDSKWPWQVSLHFGTTHIC |
| NOV12o | NSTIQESLHRSECPSQRYISLQCSHCGLRAMTGRIVGGALASDSKWPWQVSLHFGTTHIC |
| NOV12p | NSTIQESLHRSECPSQRYISLQCSNCGLRAMTGRIVGGALASDSKWPWQVSLHFGTTHIC |
| NOV12q | ----------------------------------IVGGALASDSKWPWQVSLHFGTTHIC |
| NOV12r | NSTIQESLHRSECPSQRYISLQCSHCGLRAMTGRIVGGALASDSKWPWQVSLHFGTTHIC |
| NOV12a | GGTLIDAQWVLTAAHCFFVTREKVLEGWKVYAGTSNLHQLPEAASIAEIIINSNYTDEED |
| NOV12b | GGTLIDAQWVLTAAHCFFVTREKVLEGWKVYAGTSNLHQLPEAASIAEIIINSNYTDEED |
| NOV12c | GGTLIDAQWVLTAAHCFFVTREKVLEGWKVYAGTSNLHQLPEAASIAETIINSNYTDEED |
| NOV12d | GGTLIDAQWVLTAAHCFFVTREKVLEGWKVYAGTSNLHQLPEAASIAEIIINSNYTDEED |
| NOV12e | GGTLIDAQWVLTAAHCFFVTREKVLEGWKVYAGTSNLHQLPEAASIAEIIINSNYTDEED |
| NOV12f | GGTLIDAQWVLTAAHCFFVTREKVLEGWKVYAGTSNLHQLPEAASIAEIIINSNYTDEED |
| NOV12g | GGTLIDAQWVLTAAHCFFVTREKVLEGWKVYAGTSNLNQLPEAASIAEIIINSNYTDEED |
| NOV12h | GGTLIDAQWVLTAAHCFFVTREKVLEGWKVYAGTSNLHQLPEAASIAEIIINSNYTDEED |
| NOV12i | GGTLIDAQWVLTAAHCFFVTREKVLEGWKVYAGTSNLHQLPEAASIAEIIINSNYTDEED |
| NOV12j | GGTLIDAQWVLTAAHCFFVTREKVLEGWKVYAGTSNLHQLPEAASIAEIIINSNYTDEED |
| NOV12k | GGTLIDAQWVLTAAHCFFVTREKVLEGWKVYACTSNLNQLPEAASIAEIIINSNYTDEED |
| NOV12l | GGTLIDAQWVLTAAHCFFVTREKVLEGWKVYAGTSNLHQLPEAASIAEIIINSNYTDEED |
| NOV12m | GGTLIDAQWVLTAAHCFFVTREKVLEGWKVYAGTSNLHQLPEAASIAEIIINSNYTDEED |
| NOV12n | GGTLIDAQWVLTAAHCFFVTREKVLEGWKVYAGTSNLHQLPEAASIAEIIINSNYTDEED |
| NOV12o | GGTLIDAQWVLTAAHCFFVTREKVLEGWKVYAGTSNLHQLPEAASIAEIIINSNYTDEED |
| NOV12p | GGTLIDAQWVLTAAHCFFVTREKVLEGWKVYAGTSNLHQLPEAASIAEIIINSNYTDEED |
| NOV12q | GGTLIDAQWVLTAAHCFFVTREKVLEGWKVYAGTSNLHQLPEAASIAEIIINSNYTDEED |
| NOV12r | GGTLIDAQWVLTAAHCFFVTREKVLEGWKVYAGTSNLHQLPEAASIAEIIINSNYTDEED |
| NOV12a | DYDIALMRLSKPLTLSAHIHPACLPMHGQTFSLNETCWITGFGKTRETDDKTSPFLREVQ |
| NOV12b | DYDIALMRLSKPLTLSAHIHPACLPMHGQTFSLNETCWITGFGKTRETDDKTSPSLREVQ |
| NOV12c | DYDIALMRLSKPLTLSAHIHPACLPMHGQTFSLNETCWITGFGKTRETDDKTSPSLREVQ |
| NOV12d | DYDIALMRLSKPLTLSAHIHPACLPMHGQTFSLNETCWITGFGKTRETDDKTSPFLREVQ |
| NOV12e | DYDIALMRLSKPLTLSAHIHPACLPMHGQTFSLNETCWITGFGKTRETDDKTSPFLREVQ |
| NOV12f | DYDIALMRLSKPLTLSAHIHPACLPMHGQTFSLNETCWITGFGKTRETDDKTSPFLREVQ |
| NOV12g | DYDIALMRLSKPLTLSAHIHPACLPMNGQTFSLNETCWITGFGKTRETDDKTSPFLREVQ |
| NOV12h | DYDIALMRLSKPLTLSAHINPACLPMNGQTFSLNETCWITGFGKTRETDDKTSPFLREVQ |
| NOV12i | DYDIALMRLSKPLTLSAHIHPACLPMHGQTFSLNETCWITGFGKTRETDDKTSPFLREVQ |
| NOV12j | DYDIALMRLSKPLTLSABIHPACLPMHGQTFSLNETCWITGFGKTRETDDKTSPFLREVQ |
| NOV12k | DYDIALMRLSKPLTLSAHIHPACLPMHGQTFSLNETCWITGFGKTRETDDKTSPFLREVQ |
| NOV12l | DYDIALMRLSKPLTLSAHIHPACLPMNGQTFSLNETCWITGFGKTRETDDKTSPFLREVQ |
| NOV12m | DYOIALMRLSKPLTLSAHIHPACLPMHGQTFSLNETCWITGFGKTRETDDKTSPFLREVQ |
| NOV12n | DYDIALMRLSKPLTLSAHIHPACLPMHGQTFSLNETCWITGFGKTRETDDKTSPFLREVQ |
| NOV12o | DYDIALMRLSKPLTLSAHIHPACLPMHGQTFSLNETCWITGFGKTRETDDKTSPFLREVQ |
| NOV12p | DYDIALMRLSKPLTLSAHIHPACLPMHGQTFSLNETCWITGFGKTRETDDKTSPFLREVQ |
| NOV12q | DYDIALMRLSKPLTLSAHINPACLPMHGQTFSLNETCWITUFGKTRETDDKTSPSLREVQ |
| NOV12r | DYDIALMRLSKPLTLSAHIHPACLPMHGQTFSLNETCWITGFOKTRETDOKTSPFLREVQ |
| NOV12a | VNLIDFKKCNDYLVYDSYLTPRMMCAGDLRGGRDSCQGDSGGPLVCEQNNRWYLAGVTSW |
| NOV12b | VNLIDFKKCNDYLVYDSYLTPRNNCAGDLRGGRDSCQGDSGGPLVCEQNNRWYLAGVTSW |
| NOV12c | VNLIDFKKCNDYLVYDSYLTPRMMCAGDLRGGRDSCQGDSGGPLVCEQNNRWYLAGVTSW |
| NOV12d | VNLIDFKKCNDYLVYDSYLTPRMMCAGDLRGGRDSCQGDSGGPLVCEQNNRWYLAGVTSW |
| NOV12e | VNLIDFKKCNDYLVYDSYLTPRMMCAODLRGGRDSCQGDSGGPLVCEQNNRWYLAGVTSW |
| NOV12f | VNLIDFKKCNDYLVYDSYLTPRMMCAGDLRGGRDSCQGDSGGPLVCEQNNRWYLAGVTSW |
| NOV12g | VNLIDFKKCNDYLVYDSYLTPRMMCAGDLRGGRDSCQGDSGGPLVCEQNNRWYLAGVTSW |
| NOV12h | VNLIDFKKCNDYLVYDSYLTPRMMCAGDLRGGRDSCQGDSGGPLVCEQNNRWYLAGVTSW |
| NOV12i | VNLIDFKKCNDYLVYDSYLTPRMMCAGDLRGGRDSCQGDSGGPLVCEQNNRWYLAGVTSW |
| NOV12j | VNLIDFKKCNDYLVYDSYLTPRMMCAGDLRGGRDSCQGDSGGPLVCEQNNRWYLAGVTSW |
| NOV12k | VNLIDFKKCNDYLVYDSYLTPRMMCAGDLRGGRDSCQGDSGGPLVCEQNNRWYLAGVTSW |
| NOV12l | VNLIDFKKCNDYLVYDSYLTPRMMCAGDLRGGRDSCQGDSGGPLVCEQNNRWYLAGVTSW |
| NOV12m | VNLIDFKKCNDYLVYDSYLTPRMMCAGDLRGGRDSCQGDSGGPLVCEQNNRWYLAGVTSW |
| NOV12n | VNLIDFKKCNDYLVYDSYLTPRMMCAGDLRGGRDSCQGDSGGPLVCEQNNRWYLAGVTSW |
| NOV12o | VNLIDFKKCNDYLVYDSYLTPRMMCAGDLRGGRDSCQGDSGGPLVCEQNNRWYLAGVTSW |
| NOV12p | VNLIDFKKCNDYLVYDSYLTPRMMCAGDLRGGRDSCQGDSGGPLVCEQNNRWYLAGVTSW |
| NOV12q | VNLIDFKKCNDYLVYDSYLTPRMMCAGDLRGGRDSCQGDSGGPLVCEQNNRWYLAGVTSW |
| NOV12r | VNLIDFKKCNDYLVYDSYLTPRNMCAGDLRGGRDSCQGDSGGPLVCEQNNRWYLAGVTSW |
| NOV12a | GTGCGQRNKPGVYTKVTEVLPWIYSKMEVRSLQQDTAPSRLGTSSGGDPGGAPRV--- |
| NOV12b | GTGCOQRNKPGVYTKVTEVLPWIYSLE------------------------------- |
| NOV12c | GTGCGQRNKPGVYTKVTEVLPWIYSLE------------------------------- |
| NOV12d | GTGCGQRNKPGVYTKVTEVLFWIYSKMEVRSLQQDTAPSRLGTSSGGDPGGAPRVLE- |
| NOV12e | GTGCGQRNKPGVYTKVTEVLPWIYSKMEVRSLQQDTAPSRLGTSSGGDPGGAPRVLEG |
| NOV12f | GTGCGQRNKPGVYTKVTEVLPWIYSKMEVRSLQQDTAPSRLGTSSGGDPCGAFRVLEG |
| NOV12g | GTGCGQRNKPGVYTKVTEVLPWIYSKMEVKSLQQDTAPSRLGTSSOGDPGGAPRVLEG |
| NOV12h | GTGCGQRNKPGVYTKVTEVLPWIYSKMEVRSLQQDTAPSRLGTSSGODPGGAPRVLEG |
| NOV12i | GTGCGQRNKPGVYTKVTEVLPWIYSKMEVRSLQQDTAPSRLGTSSGGDPGGAPRVLEG |
| NOV12j | GTGCGQRNKPGVYTKVTEVLPWIYSKMEVRSLQQDTAPSRLGTSSGGDPGGAPRV--- |
| NOV12k | GTGCGQRNKPGVYTKVTEVLPWIYSKMEVRSLQQDTAPSRLGTSSGGDPGGAPRV--- |
| NOV12l | GTGCGQRNKPCVYTKVTEVLPWIYSKMEVRSLQQDTAPSRLGTSSGGDPGGAPRVLEG |
| NOV12m | GTGCOQRNKPGVYTKVTEVLPWIYSKMESEVRFTKS---------------------- |
| NOV12n | GTGCGQRNKPGVYTKVTEVLPWIYSKMESEVRFRKSLEG------------------- |
| NOV12o | GTGCGQRNKPGVYTKVTEVLPWIYSKMESEVRFTKS---------------------- |
| NOV12p | GTGCOQRNKPGVYTKVTEVLPWIYSKMESEVRFTKS---------------------- |
| NOV12q | GTGCGQRNKPGVYTKVTEVLPWIYS--------------------------------- |
| NOV12r | GTGCGQRNKPGVYTKVTEVLPWIYSKMEVRSLQQDTAPSRLGTSSGGDPGGAPRVLEG |
| NOV12a | (SEQ ID NO: 104) |
| NOV12b | (SEQ ID NO: 106) |
| NOV12c | (SEQ ID NO: 108) |
| NOV12d | (SEQ ID NO: 110) |
| NOV12e | (SEQ ID NO: 112) |
| NOV12f | (SEQ ID NO: 114) |
| NOV12g | (SEQ ID NO: 116) |
| NOV12h | (SEQ ID NO: 118) |
| NOV12i | (SEQ ID NO: 120) |
| NOV12j | (SEQ ID NO: 122) |
| NOV12k | (SEQ ID NO: 124) |
| NOV12l | (SEQ ID NO: 126) |
| NOV12m | (SEQ ID NO: 128) |
| NOV12n | (SEQ ID NO: 130) |
| NOV12o | (SEQ ID NO: 132) |
| NOV12p | (SEQ ID NO: 134) |
| NOV12q | (SEQ ID NO: 136) |
| NOV12r | (SEQ ID NO: 138) |

Further analysis of the NOV12a protein yielded the following properties shown in Table 12C.

TABLE 12C
Protein Sequence Properties NOV12a

| SignalP | No Known Signal Sequence Predicted | | | | |
|---|---|---|---|---|---|
| analysis: | | | | | |
| PSORT II | PSG: a new signal peptide prediction method | | | | |
| analysis: | N-region: length 4; pos. chg 1; neg. chg 2 | | | | |
| | H-region: length 8; peak value 0.00 | | | | |
| | PSG score: −4.40 | | | | |
| | GvH: von Heijne's method for signal seq. recognition | | | | |
| | GvH score (threshold: −2.1): −6.21 | | | | |
| | possible cleavage site: between 24 and 25 | | | | |
| | >>> Seems to have no N-terminal signal peptide | | | | |
| | ALOM: Klein et al's method for TM region allocation | | | | |
| | Init position for calculation: 1 | | | | |
| | Tentative number of TMS(s) for the threshold 0.5: 1 | | | | |
| | Number of TMS(s) for threshold 0.5: 1 | | | | |
| | INTEGRAL | Likelihood = | −15.71 | Transmembrane | 168-184 |

| PERIPHERAL | Likelihood = | 2.86 (at 354) | |
|---|---|---|---|
| ALOM score: −15.71 (number of TMSs: 1) | | | |
| MTOP: Prediction of membrane topology (Hartmann et al.) | | | |
| Center position for calculation: 175 | | | |
| Charge difference: 0.5 C (2.5)-N (2.0) | | | |
| C > N: C-terminal side will be inside | | | |
| >>> membrane topology: type 1b (cytoplasmic tail 168 to 586) | | | |
| MITDISC: discrimination of mitochondrial targeting seq | | | |
| R content: | 1 | Hyd Moment(75): | 10.42 |
| Hyd Moment(95): | 7.40 | G content: | 0 |
| D/E content: | 2 | S/T content: | 0 |

```
Score: −5.39
Gavel: prediction of cleavage sites for mitochondrial preseq
cleavage site motif not found
NUCDISC: discrimination of nuclear localization signals
pat4: none
pat7: none
bipartite: none
content of basic residues: 10.2%
NLS Score: −0.47
KDEL: ER retention motif in the C-terminus: none
ER Membrane Retention Signals:
XXRR-like motif in the N-terminus: ERDS
none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: too long tail
Dileucine motif in the tail: found
LL at 173
LL at 174
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: nuclear
Reliability: 94.1
COIL: Lupas's algorithm to detect coiled-coil regions
total: 0 residues
Final Results (k = 9/23):
69.6% nuclear
13.0% mitochondrial
  4.3% vacuolar
  4.3% plasma membrane
  4.3% cytoplasmic
  4.3% vesicles of secretory system
>> prediction for CG50949-03 is nuc (k = 23)
```

A search of the NOV12a protein against the Geneseq database, a proprietary database that contains sequences published in patents and patent publication, yielded several homologous proteins shown in Table 12D.

TABLE 12D
Geneseq Results for NOV12a

| | | | Identities/ | |
|---|---|---|---|---|
| | | | Similarities for | |
| Geneseq | Protein/Organism/Length | NOV12a Residues/ | the Matched | Expect |
| Identifier | [Patent #, Date] | Match Residues | Region | Value |
| AAE01944 | Human transmembrane serine | 1 . . . 568 | 556/568 (97%) | 0.0 |
| | protease (Endotheliase 2-L) protein - | 1 . . . 563 | 559/568 (97%) | |
| | Homo sapiens, 688 aa. | | | |
| | [WO200136604-A2, 25 MAY 2001] | | | |
| AAE17238 | Human transmembrane serine | 1 . . . 559 | 554/559 (99%) | 0.0 |
| | protease - Homo sapiens, 562 aa. | 1 . . . 554 | 554/559 (99%) | |
| | [WO200196538-A2, 20 DEC. 2001] | | | |
| AAE01943 | Human transmembrane serine | 1 . . . 559 | 554/559 (99%) | 0.0 |
| | protease (Endotheliase 2-S) protein - | 1 . . . 554 | 554/559 (99%) | |
| | Homo sapiens, 562 aa. | | | |
| | [WO200136604-A2, 25 MAY 2001] | | | |
| AAU82746 | Amino acid sequence of novel | 1 . . . 559 | 523/564 (92%) | 0.0 |
| | human protease #45 - Homo sapiens, | 1 . . . 529 | 523/564 (92%) | |
| | 537 aa. [WO200200860-A2, | | | |
| | 03 JAN. 2002] | | | |
| AAB85042 | Human SER6 protein sequence - | 148 . . . 559 | 412/412 (100%) | 0.0 |
| | Homo sapiens, 421 aa. | 2 . . . 413 | 412/412 (100%) | |
| | [WO200136645-A2, 25 MAY 2001] | | | |

In a BLAST search of public sequence databases, the NOV12a protein was found to have homology to the proteins shown in the BLASTP data in Table 12E.

TABLE 12E
Public BLASTP Results for NOV12a

| | | | Identities/ | |
|---|---|---|---|---|
| Protein | | | Similarities for | |
| Accession | | NOV12a Residues/ | the Matched | Expect |
| Number | Protein/Organism/Length | Match Residues | Portion | Value |
| Q9BYE2 | Membrane-type mosaic serine | 1 . . . 586 | 577/586 (98%) | 0.0 |
| | protease - Homo sapiens | 1 . . . 581 | 579/586 (98%) | |
| | (Human), 581 aa. | | | |
| CAC41221 | Sequence 5 from Patent | 1 . . . 568 | 556/568 (97%) | 0.0 |
| | WO0136604 - Homo sapiens | 1 . . . 563 | 559/568 (97%) | |
| | (Human), 688 aa. | | | |
| CAC41220 | Sequence 3 from Patent | 1 . . . 559 | 554/559 (99%) | 0.0 |
| | WO0136604 - Homo sapiens | 1 . . . 554 | 554/559 (99%) | |
| | (Human), 562 aa. | | | |
| AAO38062 | Transmembrane protease serine 6 - | 1 . . . 559 | 551/559 (98%) | 0.0 |
| | Homo sapiens (Human), 558 aa. | 1 . . . 554 | 553/559 (98%) | |
| Q9BYE1 | Mosaic serine protease - Homo | 1 . . . 559 | 520/564 (92%) | 0.0 |
| | sapiens (Human), 537 aa. | 1 . . . 529 | 521/564 (92%) | |

PFam analysis predicts that the NOV12a protein contains the domains shown in the Table 12F.

TABLE 12F
Domain Analysis of NOV12a

| | | Identities/ | |
|---|---|---|---|
| | NOV12a | Similarities for | |
| Pfam | Match | the Matched | Expect |
| Domain | Region | Region | Value |
| 1d1_recept_a | 202 . . . 228 | 10/43 (23%) | 0.45 |
| | | 17/43 (40%) | |
| SRCR | 240 . . . 315 | 17/116 (15%) | 0.16 |
| | | 52/116 (45%) | |
| trypsin | 326 . . . 554 | 98/266 (37%) | 6.8e−83 |
| | | 187/266 (70%) | |

### Example 13

The NOV13 clone was analyzed, and the nucleotide and encoded polypeptide sequences are shown in Table 13A.

TABLE 13A
NOV13 Sequence Analysis

NOV13a,
CG51018-01 SEQ ID NO: 145 3447 bp
DNA Sequence ORF Start: ATG at 55 ORF Stop: TGA at 2932
GGTAGCCGACGCGCCGGCCGGCGCGTGACCTTGCCCCTCTTGCTCGCCTTGAAAATGGAAAAGATGCT
CGCAGGCTGCTTTCTGCTGATCCTCGGACAGATCGTCCTCCTCCCTGCCGAGGCCAGGCAGCGGTCAC
GTGGGAGGTCCATCTCTAGGGGCAGACACCCTCGGACCCACCCGCAGACGGCCCTTCTGGAGAGTTCC
TGTGAGAACAAGCGGGCAGACCTGGTTTTCATCATTGACAGCTCTCGCAGTGTCAACACCCATGACTA
TGCAAAGGTCAAGGAGTTCATCGTGGACATCTTGCAATTCTTGGACATTGGTCCTGATGTCACCCGAG
TGGGCCTGCTCCAATATGGCAGCACTGTCAAGAATGAGTTCTCCCTCAAGACCTTCAAGAGGAAGTCC
GAGGTCGAGCGTGCTGTCAAGAGGATGCGGCATCTCTCCACGGGCACCATGACTGGGCTGGCCATCCA
GTATGCCCTGAACATCGCATTCTCAGAAGCAGAGGGGGCCCGGCCCCTGAGGGAGAATGTGCCACGGG
TCATAATGATCGTGACAGATGGGAGACCTCAGGACTCCGTGGCCGAGGTGGCTGCTAAGGCACGGGAC
ACGGGCATCCTAATCTTTGCCATTGGTGTGGGCCAGGTAGACTTCAACACCTTGAAGTCCATTGGGAG
TGAGCCCCATGAGGACCATGTCTTCCTTGTGGCCAATTTCAGCCAGATTGAGACGCTGACCTCCGTCT
TCCAGAAGAAGTTGTGCACGGCCCACATGTGCAGCACCCTGGAGCATAACTGTGCCCACTTCTGCATC
AACATCCCTGGCTCATACGTCTGCAGGTGCAAACAAGGCTACATTCTCAACTCGGATCAGACGACTTG
CAGAATCCAGGATCTGTGTGCCATGGAGGACCACAACTGTGAGCAGCTCTGTGTGAATGTGCCGGGCT
CCTTCGTCTGCCAGTGCTACAGTGGCTACGCCCTGGCTGAGGATGGGAAGAGGTGTGTGGCTGTGGAC
TACTGTGCCTCAGAAAACCACGGATGTGAACATGAGTGTGTAAATGCTGATGGCTCCTACCTTTGCCA
GTGCCATGAAGGATTTGCTCTTAACCCAGATGAAAAAACGTGCACAAAGATAGACTACTGTGCCTCAT
CTAATCATGGATGTCAGTACGAGTGTGTTAACACAGATGATTCCTATTCCTGCCACTGCCTGAAAGGC
TTTACCCTGAATCCAGATAAGAAAACCTGCAGAAGGATCAACTACTGTGCACTGAACAAACCGGGCTG
TGAGCATGAGTGCGTCAACATGGAGCAGAGCTACTACTGCCGCTGCCACCGTGGCTACACTCTGGACC
CCAATGGCAAACCCTGCAGCCGAGTGGACCACTGTGCACAGCAGGACCATGGCTGTGAGCAGCTGTGT
CTGAACACGGAGCATTCCTTCGTCTGCCAGTCCTCAGAAGGCTTCCTCATCAACGAGGACCTCAAGAC
CTGCTCCCGGGTGGATTACTGCCTGCTGAGTGACCATGGTTGTGAATACTCCTGTGTCAACATGGACA
GATCCTTTGCCTGTCAGTGTCCTGAGGGACACGTGCTCCGCAGCGATGGGAAGACGTGTGCAAAATTG
GACTCTTGTGCTCTGGGGGACCACGGTTGTGAACATTCGTGTGTAAGCAGTGAAGATTCGTTTGTGTG
CCAGTGCTTTGAAGGTTATATACTCCGTGAAGATGGAAAAACCTGCAGAAGGAAAGATGTCTGCCAAG
CTATAGACCATGGCTGTGAACACATTTGTGTGAACAGTGACGACTCATACACGTGCGAGTGCTTGGAG
GGATTCCGGCTCACTGAGGATGGGAAACGCTGCCGAATTTCCTCAGGGAAGGATGTCTGCAAATCAAC
CCACCATGGCTGCGAACACATTTGTGTTAATAATGGGAATTCCTACATCTGCAAATGCTCAGAGGGAT
TTGTTCTAGCTGAGGACGGAAGACGGTGCAAGAAATGCACTGAAGGCCCAATTGACCTGGTCTTTGTG
ATCGATGGATCCAAGAGTCTTGGAGAAGAGAATTTTGAGGTCGTGAAGCAGTTTGTCACTGGAATTAT
AGATTCCTTGACAATTTCCCCCAAAGCCGCTCGAGTGGGGCTGCTCCAGTATTCCACACAGGTCCACA
CAGAGTTCACTCTGAGAAACTTCAACTCAGCCAAAGACATGAAAAAAGCCGTGGCCCACATGAAATAC
ATGGGAAAGGGCTCTATGACTGGGCTGGCCCTGAAACACATGTTTGAGAGAAGTTTTACCCAAGGAGA
AGGGGCCACGCCCTTTTCCACAAGGGTGCCCAGACCAGCCATTGTGTTCACCGACGGACGGGCTCAGG
ATGACGTCTCCGAGTGGGCCAGTAAAGCCAAGGCCAATGGTATCACTATGTATGCTGTTGGGGTAGGA
AAAGCCATTGAGGAGGAACTACAAGAGATTGCCTCTGAGCCCACAAACAAGCATCTCTTCTATGCCGA
AGACTTCAGCACAATCGATGAGATAAGTGAAAAACTCAAGAAAGGCATCTGTGAAGCTCTAGAAGACT
CCGATGGAAGACAGGACTCTCCAGCAGGGGAACTGCCAAAAACGGTCCAACAGCCAACAGAAGCCCTT
CCAGTCACCATAAATATCCAAGACCTACTTTCCTGTTCTAATTTTGCAGTGCAACACAGATATCTGTT
TGAAGAAGACAATCTTTTACGGTCTACACAAAAGCTTTCCCATTCAACAAAACCTTCAGGAAGCCCTT
TGGAAGAAAAACACGATCAATGCAAATGTGAAAACCTTATAATGTTCCAGAACCTTGCAAACGAAGAA
GTAAGAAAATTAACACAGCGCTTAGAAGAAATGACACAGAGAATGGAAGCCCTGGAAAATCGCCTGAG
ATACAGATGAAGATTAGAAATCGCGACACATTTGTAGTCATTGTATCACGGATTACAATGAACGCAGT
GCAGAGCCCCAAAGCTCAGGCTATTGTTAAATCAATAATGTTGTGAAGTAAAACAATCAGTACTGAGA
AACCTGGTTTGCCACAGAACAAAGACAAGAAGTATACACTAACTTGTATAAATTTATCTAGGAAAAAA
ATCCTTCAGAATTCTAAGATGAATTTACCAGGTGAGAATGAATAAGCTATGCAAGGTATTTTGTAATA
TACTGTGGACACAACTTGCTTCTGCCTCATCCTGCCTTAGTGTGCAATCTCATTTGACTATACGATAA
AGTTTGCACAGTCTTACTTCTGTAGAACACTGGCCATAGGAAATGCTGTTTTTTTGTACTGGACTTTA
CCTTGATATATGTATATGGATGTATGCATAAAATCATAGGACATATGTACTTGTGGAACAAGTTGGAT
TTTTTATACAATATTAAAATTCACCACTTCAGAGAAAAGTAAAAAAA
NOV13a,
CG51018-01
Protein Sequence SEQ ID NO: 146 959 aa MW at 107109.9 kD
MEKMLAGCFLLILGQIVLLPAEARQRSRGRSISRGRHARTHPQTALLESSCENKRADLVFIIDSSRSV
NTHDYAKVKEFIVDILQFLDIGPDVTRVGLLQYGSTVKNEFSLKTFKRKSEVERAVKRMRHLSTGTMT
GLAIQYALNIAFSEAEGARPLRENVPRVIMIVTDGRPQDSVAEVAAKARDTGILIFAIGVGQVDFNTL
KSIGSEPHEDHVFLVANFSQIETLTSVFQKKLCTAHMCSTLEHNCAHFCINIPGSYVCRCKQGYILNS
DQTTCRIQDLCAMEDHNCEQLCVNVPGSFVCQCYSGYALAEDGKRCVAVDYCASENHGCEHECVNADG
SYLCQCHEGFALNPDEKTCTKIDYCASSNHGCQYECVNTDDSYSCHCLKGFTLNPDKKTCRRINYCAL
NKPGCEHECVNMEESYYCRCHRGYTLDPNGKPCSRVDHCAQQDHGCEQLCLNTEDSFVCQCSEGFLIN
EDLKTCSRVDYCLLSDHGCEYSCVNMDRSFACQCPEGHVLRSDGKTCAKLDSCALGDHGCEHSCVSSE
DSFVCQCFEGYILREDGKTCRRKDVCQAIDHGCEHICVNSDDSYTCECLEGFRLTEDGKRCRISSGKD
VCKSTHHGCEHICVNNGNSYICKCSEGFVLAEDGRRCKKCTEGPIDLVFVIDGSKSLGEENFEVVKQF
VTGIIDSLTISPKAARVGLLQYSTQVHTEFTLRNFNSAKDMKKAVAHMKYMGKGSMTGLALKHMFERS
FTQGEGARPFSTRVPRAATVFTDGRAQDDVSEWASKAKANGITMYAVGVGKAIEEELQEIASEPTNKH
LFYAEDFSTMDEISEKLKKGICEALEDSDGRQDSPAGELPKTVQQPTESEPVTINIQDLLSCSNFAVQ
HRYLFEEDNLLRSTQKLSHSTKPSGSPLEEKHDQCKCENLIMFQNLANEEVRKLTQRLEEMTQRMEAL
ENRLRYR
NOV13b,
274051273 SEQ ID NO: 147 1708 bp
DNA Sequence ORF Start: at 1 ORF Stop: TGA at 850
GCCCTCGAGGGATTTTCTGAATCGCACCTCGCTCTCCATCTTGCTGTAAATCCAGGGAAGAACTTCTG
TCACTTTGGTGTACACACCAGGTTTGTTTCTCTGGCCACAGCCTGTGCCCCAGCTGGTGACACCTGCC
AGGTACCAGCGGTTGTTCTGCTCACAGACAAGAGGCCCCCCGCTGTCTCCCTGGCAGGAGTCTCTGCC
CCCACGAAGGTCCCCAGCACACATCATCCTTGGGGTAAGGTAACTCTCATAGACCAAGTAGTCATTGC
ATTTCTTGAAGTCGATGACATTGACCTGCACCTCCCGGAGGAAGGGGGATGTCTTGTCATCTGTCTCC
CTGGTCTTGCCAAAGCCTGTGATCCAGCAGGTCTCATTGAGGCTAAAGGTCTGTCCATGCATGGGGAG
GCAAGCAGGGTGGATGTGAGCGGACAGGGTCAGGGGCTTGGACAGCCGCATGAGGGCGATGTCATAGT
CGTCCTCCTCATCGGTGTAATTGCTGTTGATGATGATCTCGGCAATGGAGGCTGCCTCAGGCAACTGG
TGCAGGTTGCTGGTGCCCGCGTACACCTTCCAGCCCTCCAGGACCTTCTCCCGGGTCACGAAGAAGCA
GTGGGCGGCAGTGAGCACCCACTGGGCGTCAATGAGCGTGCCTCCACAGATGTGCGTGGTGCCGAAGT
GCAGACTCACTTGCCAAGGCCACTTGCTATCCGAGGCCAGCGCCCCTCCCACGATCCGCCCGGTCATG
GCCCTCAGTCCGCAGTGGGAACACTGGAGGGAGATATACCGCTGGGAAGGGCATTCAGACCTGTGGAG
GCTTTCCTGGATGGTGGAGTTGTATCTCAAGATTGAGAAGCTGTTGGCAAAATCCCTGTGGGCAACCT
CGGTTGTCCGGTGAGCACTCTCGAAACCCAGCTGCTGGCAGGTCTTCTCTGAGTAGGAGTCATTCCAG
TTGCTGCTACAGATGGGAAGCCACTGATGGGAGGACCCAGAGTAGATTTTAAGCAGAGACTTGTCCCA
CTCAAACCTCACGCAGCCCAGCTCGTCACTCTTCAGCTTGCAGTCCACCACCCCGTCACAGCGAACAG
CGTGCTTGGGACAGCTCTCCCTCTGCTCCTTGTACCTGATCCCTGTGTGGCCCTGCCAGAACTGGAAG
AGGATGATGAGCGAAACCACCAGGGCAATGAGGAGGAGCACGCACCCGATGAGCGGTAGCTGCTTCTG
GCCCTCCCGCCAGGTGAACTTCGGCAGGCTCGTACCTGGCCTCTCCCTGGTGGCCCTGGTTGCTGGTG
CTGACCTGGCAGGAGATGATCGCATGGGTACAGCCCCCACTGGTCTTGCTCTAACAAGGTACACTCTG
GTTGGGGAGGTTGTCACCGAGGCTGACCTGGCGGATGATGACCTGCCGGATGAGGACCTGGAAAGTGA
TGCCAGAGCCGGAGATGCCCGGGCTGGAGATGCCTGGGCTGGAGATGCCCGGCCTGGAGATGCCCGCC
CTGGAGGTGTACCAGCTGGAGATGCCTGGGCTGGAGATGCCCGGCCCGGAGGTGTCCCACCTGGAGAT
GCCTGGGCTGGAGATGCCTGGGCTGGAGATGCCCGGCCTGGAGGTGTCCCAGCTGGAGATGCCTGGGC
TGGAGATGCTCCAGCTGAAGGTGTTCTTGCTGGAGATGCATTCCCGTGGCTGTCCCTCTCCATGGTGG
ATCCGGTG
NOV13b,
274051273
Protein Sequence SEQ ID NO: 148 283 aa MW at 29179.9 kD
ALEGFSESHLALHLAVNPGKNFCHFGVHTRFVSLATACAPAGDTCQVPAVVLLTDKRPPAVSLAGVSA
PTKVPSTHHPWGKVTVIDQVVIAFLEVDELDLHLPEEGGCLVICLPGLAKACDPAGLIEAKGLSMHGE
ASRVDVSGQGQGLGQPHEGDVIVVLLIGVIAVDDDLGNGGCLRQLVQVAGARVHLPALQDLLPGHEEA
VGGSEHPLGVNERASTDVGGAEVQTHLPRPLAIRGQRPSHDPPGHGPQSAVGTLEGDTPLGRAFRPVE
AFLDGGVVSQD
NOV13c,
274051251 SEQ ID NO: 149 2893 bp
DNA Sequence ORF Start: at 2 ORF Stop: end of sequence
CACCAGATCTCCCACCATGGAAAAGATGCTCGCAGGCTGCTTTCTGCTGATCCTCGGACAGATCGTCC
TCCTCCCTGCCGAGGCCAGGGAGCGGTCACGTGGGAGGTCCATCTCTAGCGGCAGACACGCTCGGACC
CACCCGCAGACGGCCCTTCTGGAGAGTTCCTGTGAGAACAAGCGGGCAGACCTGGTTTTCATCATTGA
CAGCTCTCGCAGTGTCAACACCCATGACTATGCAAAGGTCAAGGAGTTCATCGTGGACATCTTGCAAT
TCTTGGACATTGGTCCTGATGTCACCCGAGTGGGCCTGCTCCAATATGGCAGCACTGTCAAGAATGAG
TTCTCCCTCAAGACCTTCAAGAGGAAGTCCGAGGTGGAGCGTGCTGTCAAGAGGATGCGGCATCTGTC
CACGGGCACCATGACTGGGCTGGCCATCCAGTATGCCCTGAACATCGCATTCTCAGAAGCAGAGGGGG
CCCGGCCCCTGAGGGAGAATGTGCCACGGGTCATAATGATCGTGACAGATGGGAGACCTCAGGACTCC
GTGGCCGAGGTGGCTGCTAAGGCACGGGACACGGGCATCCTAATCTTTGCCATTGGTGTGGGCCAGGT
AGACTTCAACACCTTGAAGTCCATTGGGAGTGAGCCCCATGAGCACCATGTCTTCCTTGTGGCCAATT
TCAGCCAGATTGAGACGCTGACCTCCGTGTTCCACAAGAAGTTGTGCACGGCCCACATGTGCAGCACC
CTGGAGCATAACTCTGCCCACTTCTGCATCAACATCCCTGGCTCATACGTCTGCAGGTGCAAACAAGG
CTACATTCTCAACTCGGATCAGACGACTTGCAGAATCCAGGATCTGTGTGCCATGGAGGACCACAACT
GTGAGCAGCTCTGTGTGAATGTGCCGGGCTCCTTCGTCTGCCAGTGCTACAGTGGCTACGCCCTGGCT
GAGGATGGGAAGAGGTGTGTGGCTGTGGACTACTGTGCCTCAGAAAACCACGGATGTGAACATGAGTG
TGTAAATGCTGATGGCTCCTACCTTTGCCAGTGCCATGAAGGATTTGCTCTTAACCCAGATGAAAAAA
CGTGCACAAAGATAGACTACTGTGCCTCATCTAATCACGGATGTCAGCACGAGTGTGTTAACACAGAT
GATTCCTATTCCTGCCACTGCCTGAAAGGCTTTACCCTGAATCCAGATAAGAAAACCTGCAGAAGGAT
CAACTACTGTGCACTGAACAAACCGGGCTGTGAGCATGAGTGCGTCAACATGGAGGAGAGCTACTACT
GCCGCTGCCACCGTGGCTACACTCTGGACCCCAATGGCAAAACCTGCAGCCGAGTGGACCACTGTGCA
CACCAGGACCATGGCTGTGAGCAGCTGTGTCTGAACACGGAGGATTCCTTCGTCTGCCAGTGCTCAGA
AGGCTTCCTCATCAACGAGGACCTCAAGACCTGCTCCCGGGTGGATTACTGCCTGCTGAGTGACCATG
GTTGTGAATACTCCTGTGTCAACATGGACAGATCCTTTGCCTGTCAGTGTCCTGAGGGACACGTGCTC
CGCAGCGATGGGAAGACGTGTGCAAAATTGGACTCTTGTGCTCTGGGGGACCACGGTTGTGAACATTC
GTGTGTAAGCAGTGAAGATTCGTTTGTGTGCCAGTGCTTTGAAGGTTATATACTCCGTGAAGATGGAA
AAACCTGCAGAAGGAAAGATGTCTGCCAAGCTATAGACCATGGCTGTGAACACATTTGTGTGAACAGT
GACGACTCATACACGTGCGAGTCCTTGGAGGGATTCCGGCTCGCTGAGGATGGGAAACGCTGCCGAAG
GAAGGATGTCTGCAAATCAACCCACCATGGCTGCGAACACATTTGTGTTAATAATGGGAATTCCTACA
TCTGCAAATGCTCACAGGGATTTGTTCTAGCTGAGGACGGAAGACGGTGCAAGAAATGCACTGAAGGC
CCAATTGACCTGGTCTTTGTGATCGATGGATCCAAGAGTCTTGGAGAAGAGAATTTTGAGGTCGTGAA
GCAGTTTGTCACTGGGATTATAGATTCCTTGACAATTTCCCCCAAAGCCGCTCGAGTGGGGCTGCTCC
AGTATTCCACACAGGTCCACACAGAGTTCACTCTGAGAAACTTCAACTCAGCCAAAGACATGAAAAAA
GCCGTGGCCCACATGAAATACATGGGAAAGGGCTCTATGACTGGGCTGGCCCTGAAACACATGTTTGA
GAGAAGTTTTACCCAAGGAGAAGGGGCCAGGCCCCTTTCCACAAGGGTGCCCAGAGCAGCCATTGTGT
TCACCGACGGACGGGCTCAGGATGACGTCTCCGAGTGGGCCAGTAAAGCCAAGGCCAATGGTATCACT
ATCTATGCTGTTGGGGTAGGAAAAGCCATTGAGGAGGAACTACAAGAGATTGCCTCTGACCCCACAAA
CAAGCATCTCTTCTATGCCGAAGACTTCAGCACAATGGATGAGATAAGTGAAAAACTCAAGAAAGGCA
TCTGTGAAGCTCTAGAAGACTCCGATGGAAGACAGGACTCTCCAGCAGGGGAACTGCCAAAAACGGTC
CAACAGCCAACAGAATCTGAGCCAGTCACCATAAATATCCAAGACCTACTTTCCTGTTCTAATTTTGC
AGTGCAACACAGATATCTGTTTGAAGAAGACAATCTTTTACGGTCTACACAAAAGCTTTCCCATTCAA
CAAAACCTTCAGGAAGCCCTTTGGAAGAAAAACACGATCAATGCAAATGTGAAAACCTTATAATGTTC
CAGAACCTTGCAAACGAAGAAGTAAGAAAATTAACACAGCGCTTAGAAGAAATGACACAGAGAATGGA
AGCCCTGGAAAATCGCCTGAGATACAGAGTCGACGGC
NOV13c,
274051251
Protein Sequence SEQ ID NO: 150 964 aa MW at 107650.5 kD
TRSPTMEKMLAGCFLLILGQIVLLPAEARERSRGRSISRGRHARTHPQTALLESSCENKRADLVFIID
SSRSVNTHDYAKVKEFIVDILQFLDIGPDVTRVGLLQYGSTVKNEFSLKTFKRKSEVERAVKRMRHLS
TGTMTGLAIQYALNIAFSEAEGARPLRENVPRVIMIVTDGRPQDSVAEVAAKARDTGILIFAIGVGQV
DFNTLKSIGSEPHEDHVFLVANFSQIETLTSVFQKKLCTAHMCSTLEHNCAHFCINIPGSYVCRCKQG
YILNSDQTTCRIQDLCAMEDHNCEQLCVNVPCSFVCQCYSGYALAEDGKRCVAVDYCASENHGCEHEC
VNADGSYLCQCHEGFALNPDEKTCTKIDYCASSNHCCQHECVNTDDSYSCHCLKGFTLNPDKKTCRRI
NYCALNKPGCEHECVNMEESYYCRCHRGYTLDPNGKTCSRVDHCAQQDHGCEQLCLNTEDSFVCQCSE
GFLINEDLKTCSRVDYCLLSDHGCEYSCVNMDRSFACQCPEGHVLRSDGKTCAKLDSCALGDHGCEHS
CVSSEDSFVCQCFEGYILREDGKTCRRKDVCQAIDHGCEHICVNSDDSYTCECLEGFRLAEDGKRCRR
KDVCKSTHHCCEHICVNNGNSYICKCSEGFVLAEDGRRCKKCTEGPIDLVFVIDGSKSLGEENFEVVK
QFVTGIIDSLTISPKAARVGLLQYSTQVHTEFTLRNFNSAKDMKKAVAHMKYMGKGSMTGLALKHMFE
RSFTQGEGARPLSTRVPRAAIVFTDGRAQDDVSEWASKAKANGITMYAVGVGKAIEEELQEIASEPTN
KHLFYAEDFSTMDEISEKLKKGICEALEDSDGRQDSPAGELPKTVQQPTESEPVTINIQDLLSCSNFA
VQHRYLFEEDNLLRSTQKLSHSTKPSGSPLEEKHDQCKCENLIMFQNLANEEVRKLTQRLEEMTQRME
ALENRLRYRVDG
NOV13d,
274051253 SEQ ID NO: 151 2893 bp
DNA Sequence ORF Start: at 2 ORF Stop: end of sequence
CACCAGATCTCCCACCATCGAAAAGATGCTCGCAGGCTGCTTTCTGCTGATCCTCGGACAGATCGTCC
TCCTCCCTCCCGAGGCCAGGGAGCGGTCACGTGGGAGGTCCATCTCTAGGGCCAGACACGCTCGGACC
CACCCGCAGACGGCCCTTCTGGAGAGTTCCTGTGAGAACAAGCGGGCAGACCTGGTTTTCATCATTGA
CAGCTCTCGCAGTGTCAACACCCATGACTATGCAAAGGTCAACGAGTTCATCGTGGACATCTTGCAAT
TCTTGGACATTGGTCCTGATGTCACCCGAGTGGGCCTGCTCCAATATGGCAGCACTGTCAACAATGAG
TTCTCCCTCAAGACCTTCAAGAGGAAGTCCCAGGTGGAGCGTGCTGTCAAGAGGATGCGGCATCTGTC
CACGGCCACCATGACTGGGCTGGCCATCCAGTATGCCCTGAACATCGCATTCTCAGAAGCAGAGGGGG
CCCGGCCCCTGAGGGAGAATGTGCCACGGGTCATAATGATCGTGACAGATGGGAGACCTCAGGACTCC
GTGGCCGAGGTGGCTGCTAAGGCACGGGACACGCGCATCCTAATCTTTGCCATTGGTGTGGGCCAGGT
AGACTTCAACACCTTGAAGTCCATTGGGAGTGAGCCCCATGAGGACCATGTCTTCCTTGTGGCCAATT
TCAGCCAGATTGAGACGCTGACCTCCGTGTTCCAGAAGAAGTTCTGCACGGCCCACATGTGCAGCACC
CTGGAGCATAACTGTGCCCACTTCTGCATCAACATCCCTGGCTCATACGTCTGCACGTGCAAACAAGG
CTACATTCTCAACTCGGATCAGACGACTTGCAGAATCCAGGATCTGTGTGCCATGGAGGACCACAACT
GTGAGCAGCTCTGTGTGAATGTGCCGGGCTCCTTCGTCTGCCAGTGCTACAGTGGCTACGCCCTGGCT
GAGGATGGGAAGAGGTCTGTGGCTGTGGACTACTGTGCCTCAGAAAACCACGGATGTGAACATGAGTG
TGTAAATGCTGATGGCTCCTACCTTTGCCAGTGCCATGAAGGATTTGCTCTTAACCCAGATGAAAAAA
CGTGCACAAAGATAGACTACTGTGCCTCATCTAATCACGCATGTCAGCACGAGTGTGTTAACACAGAT
GATTCCTATTCCTGCCACTGCCTGAAAGCCTTTACCCTGAATCCAGATAAGAAAACCTGCAGAAGGAT
CAACTACTGTGCACTGAACAAACCCGGCTGTGAGCATGAGTGCGTCAACATGGAGGAGAGCTACTACT
GCCGCTCCCACCGTGGCTACACTCTGGACCCCAATGGCAAAACCTGCAGCCGAGTCGACCACTGTGCA
CAGCAGGACCATGGCTGTGAGCAGCTGTGTCTGAACACGGAGGATTCCTTCGTCTCCCAGTGCTCAGA
AGGCTTCCTCATCAACGAGGACCTCAAGACCTGCTCCCGGGTGGATTACTGCCTGCTGAGTGACCATG
GTTGTGAATACTCCTGTGTCAACATGGACAGATCCTTTGCCTGTCAGTGTCCTGAGGGACACGTGCTC
CCCAGCGATGGGAAGACGTGTGCAAAATTGGACTCTTGTGCTCTGGGGGACCACGGTTGTGAACATTC
GTGTGTAAGCAGTGAAGATTCGTTTGTGTGCCAGTCTTTGAAGGTTATATACTCCGTGAAGATGGAA
AAACCTGCAGAAGGAAAGATGTCTGCCAAGCTATAGACCATGGCTGTGAACACATTTGTGTGAACAGT
GACGACTCATACACGTGCGAGTGCTTGGAGGGATTCCGGCTCGCTGAGGATGGGAAACGCTGCCCAAG
GAAGGATGTCTGCAAATCAACCCACCATGGCTGCGAACACATTTGTGTTAATAATGGGAATTCCTACA
TCTGCAAATGCTCAGAGGGATTTGTTCTAGCTGAGGACCGAAGACGGTGCAAGAAATGCACTGAAGGC
CCAATTGACCTGGTCTTTGTGATCGATGGATCCAAGAGTCTTGGAGAAGAGAATTTTGAGGTCGTGAA
GCAGTTTGTCACTGGGATTATAGATTCCTTGACAATTTCCCCCAAAGCCGCTCGAGTGGGGCTGCTCC
AGTATTCCACACAGGTCCACACAGAGTTCACTCTGAGAAACTTCAACTCAGCCAAAGACATGAAAAAA
GCCGTGGCCCACATGAAATACATGGGAAAGCGCTCTATGACTGGGCTGGCCCTGAAACACATGTTTGA
GAGAAGTTTTACCCAAGGAGAAGGGGCCAGGCCCCTTTCCACAAGGGTGCCCAGAGCAGCCATTGTGT
TCACCGACGGACGGGCTCAGGATGACGTCTCCGAGTGGGCCAGTAAAGCCAAGGCCAATGGTATCACT
ATGTATGCTGTTGGGGTAGGAAAAGCCATTGAGGAGGAACTACAAGAGATTGCCTCTGAGCCCACAAA
CAAGCATCTCTTCTATGCCGAAGACTTCAGCACAATGGATGAGATAAGTGAAAAACTCAAGAAAGGCA
TCTGTGAAGCTCTAGAAGACTCCGATGGAAGACAGGACTCTCCAGCAGGGGAACTGCCAAAAACGGTC
CAACAGCCAACAGAATCTGAGCCAGTCACCATAAATATCCAAGACCTACTTTCCTGTTCTAATTTTGC
AGTGCAACACAGATATCTGTTTGAAGAAGACAATCTTTTACGGTCTACACAAAAGCTTTCCCATTCAA
CAAAACCTTCAGGAAGCCCTTTGCAAGAAAAACACGATCAATGCAAATGTGAAAACCTTATAATGTTC
CAGAACCTTGCAAACGAAGAAGTAAGAAAATTAACACAGCGCTTAGAAGAAATGACACAGAGAATGGA
AGCCCTGGAAAATCGCCTGAGATACAGAGTCCACGGC
NOV13d,
274051253
Protein Sequence SEQ ID NO: 152 964 aa MW at 107650.5 kD
TRSPTMEKMLAGCFLLILGQIVLLPAEARERSRGRSISRGRHARTHPQTALLESSCENKRADLVFIID
SSRSVNTHDYAKVKEFIVDILQFLDIGPDVTRVGLLQYGSTVKNEFSLKTFKRKSEVERAVKRMRHLS
TGTMTGLAIQYALNIAFSEAEGARPLRENVPRVINIVTDGRPQDSVAEVAAKARDTGILIFAIGVGQV
DFNTLKSIGSEPHEDHVFLVANFSQIETLTSVFQKKLCTAHMCSTLEHNCAHFCINIPGSYVCRCKQG
YILNSDQTTCRIQDLCAMEDHNCEQLCVNVPGSFVCQCYSGYALAEDGKRCVAVDYCASENHGCEHEC
VNADGSYLCQCHEGFALNPDEKTCTKIDYCASSNHGCQHECVNTDDSYSCHCLKGFTLNPDKKTCRRI
NYCALNKPGCEHECVNMEESYYCRCHRGYTLDPNGKTCSRVDHCAQQDHGCEQLCLNTEDSFVCQCSE
GFLTNEDLKTCSRVDYCLLSDHGCEYSCVNMDRSFACQCPEGHVLRSDGKTCAKLDSCALGDHGCEHS
CVSSEDSFVCQCFEGYILREDGKTCRRKDVCQAIDHGCEHICVNSDDSYTCECLEGFRLAEDGKRCRR
KDVCKSTHHGCEHICVNNGNSYICKCSEGFVLAEDGRRCKKCTEGPIDLVFVIDGSKSLGEENFEVVK
QFVTGIIDSLTISPKAARVGLLQYSTQVHTEFTLRNFNSAKDMKKAVANMKYMGKGSMTGLALKHMFE
RSFTQGEGARPLSTRVPRAAIVFTDGRAQDDVSEWASKAKANGITMYAVUVGKAIEEELQEIASEPTN
KHLFYAEDFSTNDEISEKLKKGICEALEDSDGRQDSPAGELPKTVQQPTESEPVTINIQDLLSCSNFA
VQHRYLFEEDNLLRSTQKLSHSTKPSGSPLEEKHDQCKCENLIMFQNLANEEVRKLTQRLEEMTQRME
ALENRLRYRVDG
NOV13e,
306562753 SEQ ID NO: 153 2836 bp
DNA Sequence ORF Start: at 2 ORF Stop: end of sequence
CACCAGATCTCCCACCATGGAAAAGATGCTCGCAGGCTGCTTTCTGCTGATCCTCGGACAGATCGTCC
TCCTCCCTGCCGAGGCCAGGGAGCGGTCACGTGCGAGGTCCATCTCTAGGGGCAGACACGCTCGGACC
CACCCGCAGACGGCCCTTCTGCAGAGTTCCTGTGAGAACAAGCGGGCAGACCTGGTTTTCATCATTGA
CAGCTCTCGCAGTGTCAACACCCATGACTATGCAAAGGTCAAGGAGTTCATCGTGGACATCTTGCAAT
TCTTGGACATTGGTCCTGATGTCACCCGAGTGGGCCTGCTCCAATATCGCAGCACTGTCAAGAATGAG
TTCTCCCTCAAGACCTTCAAGAGGAAGTCCGAGGTGGAGCGTGCTGTCAAGAGGATGCGGCATCTGTC
CACGGGCACCATGACCGGGCTGGCCATCCAGTATGCCCTGAACATCGCATTCTCAGAAGCAGAGGGGG
CCCGGCCCCTGAGGGAGAATGTGCCACGGGTCATAATGATCGTGACAGATGGGAGACCTCACGACTCC
GTGGCCGAGGTGGCTGCTAAGGCACGGGACACGGGCATCCTAATCTTTGCCATTGGTGTGGGCCAGGT
AGACTTCAACACCTTGAAGTCCATTGGGAGTGAGCCCCATGAGGACCATGTCTTCCTTGTGGCCAATT
TCAGCCAGATTGAGACGCTGACCTCCGTGTTCCAGAAGAAGTTGTGCACGGCCCACATGTGCAGCACC
CTGGAGCATAACTGTGCCCACTTCTGCATCAACATCCCTGGCTCATACGTCTGCAGGTGCAAACAAGG
CTACATTCTCAACTCGGATCAGACGACTTGCAGAATCCAGGATCTGTGTGCCATGGAGGACCACAACT
GTGAGCAGCTCTGTGTGAATGTGCCGGGCTCCTTCGTCTGCCAGTGCTACAGTGGCTACGCCCTGGCT
GAGGATGGGAAGAGGTGTGTGGCTGTGGACTACTGTGCCTCAGAAAACCACGGATGTGAACATGAGTG
TGTAAATGCTGATGGCTCCTACCTTTGCCAGTGCCATGAAGGATTTGCTCTTAACCCAGATGAAAAAA
CGTGCACAAAGATAGACTACTGTGCCTCATCTAATCACGGATGTCAGCACGAGTGTGTTAACACAGAT
GATTCCTATTCCTGCCACTCCCTGAAAGGCTTTACCCTGAATCCAGATAAGAAAACCTGCAGAAGGAT
CAACTACTCTGCACTGAACAAACCUGGCTGTGAGCATGAGTGCGTCAACATGGAGGAGAGCTACTACT
GCCGCTGCCACCGTGGCTACACTCTGGACCCCAATGGCAAAACCTGCAGCCGAGTGGACCACTGTGCA
CAGCAGGACCATGGCTGTGAGCAGCTGTGTCTGAACACGGAGGATTCCTTCGTCTGCCAGTGCTCAGA
AGGCTTCCTCATCAACGAGGACCTCAAGACCTGCTCCCGGGTGGATTACTGCCTGCTGAGTGACCATG
GTTGTGAATACTCCTGTGTCAACATGGACAGATCCTTTGCCTGTCAGTGTCCTGAGGGACACGTGCTC
CGCAGCGATGGGAAGACGTGTGCAAAATTGGACTCTTGTGCTCTGGGGGACCACGGTTGTGAACATTC
GTGTGTAAGCAGTGAAGATTCGTTTGTGTGCCAGTGCTTTGAAGGTTATATACTCCGTGAAGATGGAA
AAACCTGCAGAAGGAAAGATGTCTGCCAAGCTATAGACCATGGCTGTGAACACATTTGTGTGAACAGT
GACGACTCATACACGTGCGAGTGCTTGGAGGGATTCCGGCTCGCTGAGGATGGGPAACGCTGCCGAAG
CAAGGATGTCTGCAAATCAACCCACCATGGCTGCGAACACATTTGTGTTAATAATGGGAATTCCTACA
TCTGCAAATGCTCAGAGGGATTTGTTCTAGCTGAGGACGGAAGACGGTGCAAGAAATGCACTGAAGGC
CCAATTGACCTGGTCTTTGTGATCGATGGATCCAAGAGTCTTGGAGAAGAGAATTTTGAGGTCGTGAA
GCAGTTTGTCACTGGAATTATAGATTCCTTGACAATTTCCCCCAAAGCCGCTCGAGTGGGGCTGCTCC
AGTATTCCACACAGGTCCACACAGAGTTCACTCTGAGAAACTTCAACTCAGCCAAAGACATGAAAAAA
GCCGTGGCCCACATGAAATACATGGGAAAGGGCTCTATGACTGGGCTGGCCCTGAAACACATGTTTGA
GACAAGTTTTACCCAAGCAGAAGGGGCCAGGCCCCTTTCCACAAGGGTGCCCAGAGCAGCCATTGTGT
TCACCGACGGACCGGCTCAGGATGACGTCTCCGAGTGGGCCAGTAAAGCCAAGGCCAATGGTATCACT
ATGTATGCTGTTUGGGTAGGAAAAGCCATTGAGGAGCAACTACAAGAGATTGCCTCTGAGCCCACAAA
CAAGCATCTCTTCTATGCCGAAGACTTCAGCACAATGGATGAGATAAGTGAAAAACTCAAGAAAGGCA
TCTGTGAAGCTCTAGAAGACTCCGATGGAAGACAGGACTCTCCAGCAGGGGAACTGCCAAAAACGGTC
CAACAGCCAACAGTGCAACACAGATATCTGTTTGAAGAAGACAATCTTTTACGGTCTACACAAAAGCT
TTCCCATTCAACAAAACCTTCAGGAAGCCCTTTGGAAGAAAAACACGATCAATGCAAATGTGAAAACC
TTATAATGTTCCAGAACCTTGCAAACGAAGAAGTAAGAAAATTAACACAGCGCTTAGAAGAAATGACA
CAGAGAATGGAAGCCCTGGAAAAATCGCCTGAGATACAGAGTCGACGGC
NOV13e,
306562753
Protein Sequence SEQ ID NO: 154 945 aa MW at105588.3 kD
TRSPTMEKMLAGCFLLILGQIVLLPAEARERSRGRSISRGRHARTHPQTALLESSCENKRADLVFIID
SSRSVNTHDYAKVKEFIVDILQFLDIGPDVTRVGLLQYGSTVKNEFSLKTFKRKSEVERAVKRMRHLS
TGTMTGLAIQYALNIAFSEAEGARPLRENVPRVIMIVTDGRPQDSVAEVAAKARDTGILIFAIGVGQV
DFNTLKSIGSEPHEDHVFLVANFSQIETLTSVFQKKLCTAHMCSTLEHNCAHFCINIPGSYVCRCKQG
YILNSDQTTCRIQDLCAMEDHNCEQLCVNVPGSFVCQCYSGYALAEDGKRCVAVDYCASENHGCEHEC
V&ADGSYLCQCHEGFALNPDEKTCTKIDYCASSNHGCQHECVNTDDSYSCHCLKGFTLNPDKKTCRRI
NYCALNKPGCEHECVNMEESYYCRCHRGYTLDPNGKTCSRVDHCAQQDHGCEQLCLNTEDSFVCQCSE
GFLINEDLKTCSRVDYCLLSDHGCEYSCVNMDRSFACQCPEGHVLRSDGKTCAKLDSCALGDHGCEHS
CVSSEDSFVCQCFEGYILREDCKTCRRKDVCQAIDHGCEHICVNSDDSYTCECLEGFRLAEDGKRCRR
KDVCKSTHHGCEHICVNNGNSYICKCSEGFVLAEDGRRCKKCTEGPIDLVFVIDGSKSLGEENFEVVK
QFVTGIIDSLTISPKAARVGLLQYSTQVHTEFTLRNFNSAKDMKKAVAHMKYMGKGSMTGLALKHMFE
RSFTQGEGARPLSTRVPRAAIVFTDCRAQDDVSEWASKAKANGITMYAVGVGKAIEEELQELASEPTN
KHLFYAEDFSTMDEISEKLKKGICEALEDSDGRQDSPAGELPKTVQQPTVQHRYLFEEDNLLRSTQKL
SHSTKPSGSPLEEKHDQCKCENLIMFQNLANEEVRKLTQRLEEMTQRMEALENRLRYRVDG
NOV13f,
CG51018-02 SEQ ID NO: 155 2742 bp
DNA Sequence ORF Start: at 1 ORF Stop: end of sequence
AGGCAGCGGTCACGTGGGAGGTCCATCTCTAGGGGCAGACACGCTCGGACCCACCCGCAGACGGCCCT
TCTGCAGAGTTCCTGTGAGAACAAGCGGGCAGACCTGGTTTTCATCATTGACAGCTCTCGCAGTGTCA
ACACCCATGACTATGCAAAGGTCAAGGAGTTCATCGTGGACATCTTGCAATTCTTGGACATTGGTCCT
GATGTCACCCGAGTGGGCCTGCTCCAATATGGCAGCACTGTCAAGAATGAGTTCTCCCTCAAGACCTT
CAAGAGGAAGTCCGAGGTGGAGCGTGCTGTCAAGAGGATGCGGCATCTGTCCACGGGCACCATGACTG
GGCTGGCCATCCAGTATGCCCTGAACATCGCATTCTCAGAAGCAGAGGGGGCCCGGCCCCTGAGGGAG
AATGTGCCACGGGTCATAATGATCGTGACAGATGGGAGACCTCAGGACTCCGTGGCCGAGGTGGCTGC
TAAGGCACGGGACACGGGCATCCTAATCTTTGCCATTGGTGTGGGCCAGGTAGACTTCAACACCTTGA
AGTCCATTGGGAGTGAGCCCCATGAGGACCATGTCTTCCTTGTGGCCAATTTCAGCCAGATTGAGACG
CTGACCTCCGTGTTCCAGAAGAAGTTGTCCACGGCCCACATGTGCAGCACCCTGGAGCATAACTGTGC
CCACTTCTGCATCAACATCCCTGGCTCATACGTCTGCAGGTGCAAACAAGGCTACATTCTCAACTCTG
ATCAGACGACTTGCAGAATCCAGGATCTGTGTGCCATGGAGGACCACAACTGTGAGCACCTCTGTGTG
AATGTGCCGGGCTCCTTCGTCTGCCAGTGCTACAGTGGCTACGCCCTGGCTGAGGATGGGAAGAGGTG
TGTGGCTGTGGACTACTGTGCCTCAGAAAACCACGGATGTGAACATGAGTGTGTAAATGCTGATGGCT
CCTACCTTTGCCAGTGCCATGAAGGATTTGCTCTTAACCCAGATAAGAAAACGTGCACAAAGATAGAC
TACTGTGCCTCATCTAATCACGGATGTCAGCACGAGTGTGTTAACACAGATGATTCCTATTCCTGCCA
CTGTCTGAAAGGCTTTACCCTGAATCCAGATAAGAAAGCCTGCAGAAGGATCAACTACTGTGCACTGA
ACAAACCGGGCTGTGAGCATGAGTGCGTCAACATGGAGGAGAGCTACTACTCCCGCTGCCACCGTGGC
TACACTCTGGACCCCAATCGCAAAACCTGCAGCCGAGTGGACCACTGTGCACAGCAGCACCATGGCTG
TGAGCAGCTGTGTCTGAACACGGAGGATTCCTTCGTCTGCCAGTGCTCAGAAGGCTTCCTCATCAACG
AGGACCTCAAGACCTGCTCCCGGGTGGATTACTGCCTGCTGAGTGACCATGGTTGTGAATACTCCTGT
GTCAACATGGACAGATCCTTTGCCTGTCAGTGTCCTGAGGGACACGTGCTCCGCAGCGATGGGAAGAC
GTGTGCAAAATTGGACTCTTGTGCTCTGGGGGACCACGGTTGTGAACATTCGTGTGTAAGCAGTGAAG
ATTCGTTTGTGTGCCAGTGCTTTGAAGGTTATATACTCCGTGAAGATGGAAAAACCTGCAGAAGGAAA
GATGTCTGCCAAGCTATAGACCATGGCTGTCAACACATTTGTGTGAACAGTGACGACTCATACACGTG
CGAGTGCTTGGAGGGATTCCGCCTCGCTGAGGATGGGAAACGCTGCCGAAGGAAGGATGTCTGCAAAT
CAACCCACCATGGCTGCGAACACATTTGTGTTAATAATGGGAATTCCTACATCTGCAAATGCTCAGAG
GGATTTGTTCTAGCTGAGGACGGAAGACGGTGCAAGAAATGCACTGAAGGCCCAATTGACCTGGTCTT
TGTGATCGATGGATCCAAGAGTCTTGGAGAAGAGAATTTTGAGGTCGTGAAGCAGTTTGTCACTGGAA
TTATAGATTCCTTGACAATTTCCCCCAAAGCCGCTCGAGTGGCGCTGCTCCAGTATTCCACACAGGTC
CACACAGAGTTCACTCTGAGAAACTTCAACTCAGCCAAAGACATGAAAAAAGCCGTGGCCCACATGAA
ATACATGGGAAAGGGCTCTATGACTGGGCTGGCCCTGAAACACATCTTTGAGAGAAGTTTTACCCAAG
GAGAAGGGGCCACGCCCCTTTCCACAAGGGTGCCCAGAGCAGCCATTGTGTTCACCGACGGACGGGCT
CAGGATGACGTCTCCGAGTGGGCCAGTAAAGCCAAGGCCAATTGTATCACTATGTATGCTGTTGGGGT
AGGAAAAGCCATTGAGGAGGAACTACAAGAGATTGCCTCTGAGCCCACAAACAAGCATCTCTTCTATG
CCGAAGACTTCAGCACAATGGATGAGATAAGTGAAAAACTCAAGAAAGGCATCTGTGAAGCTCTAGAA
GACTCCGATGGAAGACAGGACTCTCCAGCAGGGGAACTGCCAAAAACGGTCCAACAGCCAACAGTGCA
ACACAGATATCTGTTTGAAGAAGACAATCTTTTACGGTCTACACAAAAGCTTTCCCATTCAACAAAAC
CTTCAGGAAGCCCTTTGGAAGAAAAACACGATCAATGCAAATGTGAAAACCTTATAATGTTCCAGAAC
CTTGCAAACGAAGAAGTAAGAAAATTAACACAGCGCTTAGAAGAAATGACACAGAGAATGGAAGCCCT
GGAAAATCGCCTGAGATACAGA
NOV13f,
CG51018-02
Protein Sequence SEQ ID NO: 156 914 aa MW at 102314.4 kD
RQRSRGRSISRGRHARTHPQTALLESSCENKRADLVFIIDSSRSVNTHDYAKVKEFIVDILQFLDIGP
DVTRVGLLQYGSTVKNEFSLKTFKRKSEVERAVKRMRHLSTGTMTGLAIQYALNIAFSEAEGARPLRE
NVPRVIMIVTDGRPQDSVAEVAAKARDTGILIFAIGVGQVDFNTLKSIGSEPHEDHVFLVANFSQIET
LTSVFQKKLCTAHMCSTLEHNCAHFCINIPGSYVCRCKQGYILNSDQTTCRIQDLCAMEDHNCEQLCV
NVPGSFVCQCYSGYALAEDGKRCVAVDYCASENHGCEHECVNADGSYLCQCHEGFALNPDKKTCTKID
YCASSNHGCQHECVNTDDSYSCHCLKGFTLNPDKKACRRINYCALNKPGCEHECVNMEESYYCRCHRG
YTLDPNGKTCSRVDHCAQQDHGCEQLCLNTEDSFVCQCSEGFLINEDLKTCSRVDYCLLSDHGCEYSC
VNMDRSFACQCPEGHVLRSDGKTCAKLDSCALGDHGCEHSCVSSEDSFVCQCFEGYILREDGKTCRRK
DVCQAIDHGCEHICVNSDDSYTCECLEGFRLAEDGKRCRRKDVCKSTHHGCEHICVNNGNSYICKCSE
GFVLAEDGRRCKKCTEGPIDLVFVIDGSKSLGEENFEVVKQFVTGIIDSLTISPKAARVGLLQYSTQV
HTEFTLRNFNSAKDMKKAVAHMKYMGKGSMTGLALKHIFERSFTQGEGARPLSTRVPRAAIVFTDGRA
QDDVSEWASKAKANCITMYAVGVGKAIEEELQEIASEPTNKHLFYAEDFSTMDEISEKLKKGICEALE
DSDGRQDSPAGELPKTVQQPTVQHRYLFEEDNLLRSTQKLSHSTKPSOSPLEEKHDQCKCENLIMFQN
LANEEVRKLTQRLEEMTQRMEALENRLRYR
NOV13g,
CG51018-03 SEQ ID NO: 157 2756 bp
DNA Sequence ORF Start: ATG at 3 ORF Stop: TGA at 2718
TGACCTTGCCCCTCTTGCTCGCCTTGAAAATGGAAAAGATGCTCGCAGGCTGCTTTCTGCTGATCCTC
GGACAGATCGTCCTCCTCCCTGCCGAGGCCAGGGAGCGGTCACGTGGGAGGTCCATCTCTAGGGGCAG
ACACGCTCGGACCCACCCGCAGACGGCCCTTCTGCAGAGTTCCTGTGAGAACATGCGGGCAGACCTGG
TTTTCATCATTGACAGCTCTCGCAGTGTCAACACCCATGACTATGCAAAGGTCAAGGAGTTCATCGTG
GACATCTTGCAATTCTTGGACATTGGTCCTGATGTCACCCGAGTGGGCCTGCTCCAATATGGCAGCAC
TGTCAAGAATGAGTTCTCCCTCAAGACCTTCAAGAGGAAGTCCGAGGTGGAGCGTGCTGTCAAGAGGA
TGCGCCATCTGTCCACGGGCACCATGACCGGGCTGGCCATCCAGTATGCCCTGAACATCGCATTCTCA
GAAGCAGAGGGGOCCCGGCCCCTGAGGGAGAATGTGCCACGGGTCATAATGATCGTGACAGATGGGAG
ACCTCAGGACTCCGTGGCCGAGGTCGCTGCTAAGGCACGGGACACGCGCATCCTAATCTTTGCCATTG
CTGTGGQCCAGGTAGACTTCAACACCTTGAAGTCCATTGGGAGTGAGCCCCATGAGGACCATGTCTTC
CTTGTGQCCAATTTCAGCCAGATTGAGACGCTCACCTCCGTGTTCCAGAAGAAGTTGTGCACGGCCCA
CATGTGCAGCACCCTGGAGCATAACTGTGCCCACTTCTGCATCAACATCCCTGGCTCATACGTCTGCA
GGTGCAAACAAGGCTACATTCTCAACTCGGATCAGACCACTTGCAGAATCCAGGATCTGTGTGCCATG
GAGGACCACAACTGTGAGCAGCTCTGTGTGAATGTGCCGGGCTCCTTCGTCTGCCAGTGCTACAGTGG
CTACGCCCTGGCTGAGGATGGGAAGAGGTGTGTGGCTGTGGACTACTGTGCCTCATCTAATCACGGAT
GTCAGCACGAGTGTGTTAACACAGATGATTCCTATTCCTGCCACTGCCTGAAAGGCTTTACCCTGAAT
CCAGATAAGAAAACCTGCAGAAGGATCAACTACTGTGCACTGAACAAACCGGGCTGTGAGCATGAGTG
CGTCAACATGGAGGAGAGCTACTACTCCCGCTCCCACCGTGGCTACACTCTGGACCCCAATGGCAAAA
CCTGCAGCCGAGTGGACCACTGTGCACAGCAGGACCATGGCTGTGAGCAGCTGTGTCTGAACACGGAG
GATTCCTTCGTCTGCCAGTGCTCAGAAGGCTTCCTCATCAACGAGGACCTCAAGACCTGCTCCCGGGT
GGATTACTGCCTGCTGAGTGACCATGGTTGTGAATACTCCTGTGTCAACATGGACAGATCCTTTGCCT
GTCAGTGTCCTGAGGGACACGTGCTCCGCACCGATGGGAAGACGTGTGCAAAATTGGACTCTTGTGCT
CTGGGGGACCACGGTTGTGAACATTCGTGTGTAAGCAGTGAAGATTCGTTTGTGTGCCAGTGCTTTGA
AGGTTATATACTCCGTGAAGATGGAAAAACCTGCAGAAGGAAAGATGTCTGCCAAGCTATAGACCATG
GCTGTGAACACATTTGTGTGAACAGTGATGACTCATACACGTGCGAGTGCTTGGAGGGATTCCGGCTC
GCTGAGGATGGGAAACGCTGCCGAAGGAAGGATGTCTGCAAATCAACCCACCATGGCTGCGAACACAT
TTGTGTTAATAATGGGAATTCCTACATCTGCAAATGCTCAGAGGGATTTGTTCTAGCTGAGGACGGAA
GACGGTGCAACAAATGCACTGAAGGCCCAATTGACCTGGTCTTTGTGATCGATGGATCCAACAGTCTT
GGAGAAGAGAATTTTGAGGTCGTGAACCAGTTTGTCACTGGAATTATAGATTCCTTGACAATTTCCCC
CAAAGCCGCTCGAGTGGGGCTGCTCCAGTATTCCACACAGGTCCACACAGAGTTCACTCTGAGAAACT
TCAACTCACCCAAACACATGAAAAAAGCCGTGGCCCACATGAAATACATGGGAAAGGGCTCTATGACT
GGGCTGGCCCTGAAACACATGTTTGAGAGAAGTTTTACCCAACGAGAAGGGGCCAGGCCCCTTTCCAC
AAGGGTGCCCAGACCAGCCATTGTGTTCACCGACGGACGGGCTCAGGATGACGTCTCCGAGTGGGCCA
GTAAAGCCAAGGCCAATGGTATCACTATGTATGCTGTTGGGGTAGGAAAAGCCATTGAGGAGGAACTA
CAAGAGATTGCCTCTGAGCCCACAAACAAGCATCTCTTCTATGCCGAAGACTTCAGCACAATGGATGA
GATAAGTGAAAAACTCAAGAAAGGCATCTGTGAAGCTCTAGAAGACTCCGATGGAAGACAGGACTCTC
CAGCAGGGGAACTGCCAAAAACQGTCCAACAGCCAACAGTGCAACACAGATATCTGTTTGAAGAAGAC
AATCTTTTACGGTCTACACAAAAGCTTTCCCATTCAACAAAACCTTCAGGAAGCCCTTTGGAAGAAAA
ACACGATCAATGCAAATGTGAAAACCTTATAATGTTCCAGAACCTTGCAAACGAAGAAGTAAGAAAAT
TAACACAGCGCTTAGAAGAAATGACACAGAGAATGGAAGCCCTGGAAAATCGCCTGAGATACAGATGA
AGATTAGAAATCGCGACACATTTGTAAAGGGCGAAT
NOV13g,
CG51018-03
Protein Sequence SEQ ID NO: 158 896 aa MW at 100259.6 kD
MEKMLAGCFLLILGQIVLLPAEARERSRGRSISRGRHARTHPQTALLESSCENMRADLVFIIDSSRSV
NTHDYAKVKEFIVDILQFLDIGPDVTRVGLLQYGSTVKNEFSLKTFKRKSEVERAVKRMRHLSTGTMT
GLAIQYALNIAFSEAEGARPLRENVPRVIMIVTDGRPQDSVAEVAAKARDTGILIFAIGVGQVDFNTL
KSIGSEPHEDHVFLVANFSQIETLTSVFQKKLCTAHMCSTLEHNCAHFCINIPGSYVCRCKQGYILNS
DQTTCRIQDLCAMEDHNCEQLCVNVPGSFVCQCYSGYALAEDGKRCVAVDYCASSNHGCQHECVNTDD
SYSCHCLKGFTLNPDKKTCRRINYCALNKPGCEHECVNMEESYYCRCHRGYTLDPNGKTCSRVDHCAQ
QDHGCEQLCLNTEDSFVCQCSEGFLINEDLKTCSRVDYCLLSDHGCEYSCVNMDRSFACQCPEGHVLR
SDGKTCAKLDSCALGDHGCEHSCVSSEDSFVCQCFEGYILREDGKTCRRKDVCQAIDHGCEHICVNSD
DSYTCECLEGFRLAEDGKRCRRKDVCKSTHHGCEHICVNNGNSYICKCSEGFVLAEDGRRCKKCTEGP
IDLVFVIDGSKSLGEENFEVVKQFVTGIIDSLTTSPKAARVGLLQYSTQVHTEFTLRNFNSAKDMKKA
VAHMKYMGKGSMTGLALKHMFERSFTQGEGARPLSTRVPRAAIVFTDGRAQDDVSEWASKAKANGITM
YAVGVGKAIEEELQEIASEPTNKHLFYAEDFSTMDEISEKLKKGICEALEDSDGRQDSPAGELPKTVQ
QPTVQHRYLFEEDNLLRSTQKLSHSTKPSGSPLEEKHDQCKCENLIMFQNLANEEVRKLTQRLEEMTQ
RMEALENRLRYR
NOV13h,
13374217 SNP
for CG51018-01 SEQ ID NO: 159 3447 bp SNP: 462 T/C
DNA Sequence ORF Start: ATG at 55 ORF Stop: end of sequence
GGTAGCCGACGCGCCGGCCGGCGCGTGACCTTGCCCCTCTTGCTCGCCTTGAAAATGGAAAAGATGCTCGC
AGGCTGCTTTCTGCTGATCCTCGGACAGATCGTCCTCCTCCCTGCCGAGGCCAGGCAGCGGTCACGTGGGA
GGTCCATCTCTAGGGGCAGACACGCTCGGACCCACCCGCAGACGGCCCTTCTGGAGAGTTCCTGTGAGAAC
AAGCGGCCAGACCTGGTTTTCATCATTGACAGCTCTCGCAGTGTCAACACCCATGACTATGCAAAGGTCAA
GGAGTTCATCGTGGACATCTTCCAATTCTTGGACATTGGTCCTGATGTCACCCGAGTGGGCCTGCTCCAAT
ATGGCAGCACTGTCAAGAATGAGTTCTCCCTCAAGACCTTCAAGAGGAAGTCCGAGGTGGAGCGTGCTGTC
AAGACGATGCGGCATCTGTCCACGGGCACCATGACCGGGCTGGCCATCCAGTATGCCCTGAACATCGCAAT
CTCAGAAGCAGAGGGGGCCCGGCCCCTGAGGGAGAATGTGCCACGGGTCATAATGATCGTCACAGATGGGA
GACCTCAGGACTCCGTGGCCGAGGTGGCTGCTAAGGCACGGGACACGGGCATCCTAATCTTTGCCATTGGT
GTGGGCCAGGTAGACTTCAACACCTTGAAGTCCATTGGGAGTGAGCCCCATGAGGACCATGTCTTCCTTGT
GGCCAATTTCAGCCAGATTGAGACGCTGACCTCCGTCTTCCAGAAGAAGTTGTGCACGGCCCACATCTGCA
GCACCCTGCAGCATAACTGTGCCCACTTCTGCATCAACATCCCTGGCTCATACGTCTGCAGGTGCAAACAA
GGCTACATTCTCAACTCGGATCAGACGACTTGCAGAATCCAGGATCTGTGTGCCATGGAGGACCACAACTG
TGAGCAGCTCTGTGTGAATGTGCCGGGCTCCTTCGTCTGCCAGTGCTACAGTGGCTACGCCCTGGCTGAGG
ATGGGAAGAGGTGTGTGGCTGTGGACTACTGTGCCTCAGAAAACCACGGATGTGAACATGAGTGTGTAAAT
GCTGATGGCTCCTACCTTTGCCAGTGCCATGAAGGATTTGCTCTTAACCCAGATGAAAAAACGTGCACAAA
GATAGACTACTGTGCCTCATCTAATCATGGATGTCAGTACGAGTCTGTTAACACAGATGATTCCTATTCCT
GCCACTGCCTGAAAGGCTTTACCCTGAATCCAGATAAGAAAACCTGCAGAAGGATCAACTACTGTGCACTG
AACAAACCGGGCTGTGAGCATGAGTGCGTCAACATGCAGGAGACCTACTACTGCCGCTGCCACCGTGGCTA
CACTCTGGACCCCAATGGCAAACCCTGCAGCCGAGTGGACCACTGTGCACAGCAGGACCATGGCTGTGAGC
AGCTGTGTCTGAACACGGAGGATTCCTTCGTCTGCCAGTGCTCAGAAGGCTTCCTCATCAACGAGGACCTC
AAGACCTGCTCCCGGGTGGATTACTGCCTGCTGACTGACCATGGTTGTGAATACTCCTGTGTCAACATGGA
CAGATCCTTTGCCTGTCAGTGTCCTGAGGGACACGTGCTCCGCAGCGATGGGAAGACGTGTGCAAAATTGG
ACTCTTGTGCTCTGGGGGACCACGGTTGTGAACATTCGTGTGTAAGCAGTGAAGATTCGTTTGTGTGCCAG
TGCTTTGAAGGTTATATACTCCGTGAAGATGGAAAAACCTGCAGAAGGAAAGATGTCTGCCAAGCTATAGA
CCATGGCTGTGAACACATTTGTGTGAACAGTGACGACTCATACACGTGCGAGTGCTTGGAGGGATTCCGGC
TCACTGAGGATGGGAAACGCTGCCGAATTTCCTCAGGGAAGGATGTCTGCAAATCAACCCACCATGGCTGC
GAACACATTTGTGTTAATAATGGGAATTCCTACATCTGCAAATGCTCAGAGGGATTTGTTCTAGCTGAGGA
CGGAAGACCGTGCAAGAAATGCACTGAAGGCCCAATTGACCTCGTCTTTGTGATCGATGGATCCAAGAGTC
TTGGAGAAGAGAATTTTGAGGTCGTGAAGCAGTTTGTCACTGGAATTATAGATTCCTTGACAATTTCCCCC
AAAGCCGCTCGAGTGGGGCTGCTCCAGTATTCCACACAGGTCCACACAGAGTTCACTCTGAGAAACTTCAA
CTCAGCCAAAGACATGAAAAAAGCCGTGGCCCACATGAAATACATGGGAAAGGGCTCTATGACTGGGCTGG
CCCTGAAACACATGTTTGAGAGAAGTTTTACCCAAGGAGAAGGGGCCAGGCCCTTTTCCACAAGGGTGCCC
AGAGCAGCCATTGTGTTCACCGACGCACGGGCTCAGGATGACGTCTCCGAGTGGGCCAGTAAAGCCAAGGC
CAATGGTATCACTATGTATGCTGTTGGGGTAGGAAAAGCCATTGAGGAGGAACTACAAGAGATTGCCTCTG
AGCCCACAAACAAGCATCTCTTCTATGCCGAAGACTTCAGCACAATGGATGAGATAAGTGAAAAACTCAAG
AAAGGCATCTGTGAAGCTCTAGAAGACTCCGATGGAAGACAGGACTCTCCAGCAGGGGAACTGCCAAAAAC
GGTCCAACAGCCAACAGAATCTGAGCCAGTCACCATAAATATCCAAGACCTACTTTCCTGTTCTAATTTTG
CAGTGCAACACAGATATCTGTTTGAAGAAGACAATCTTTTACGGTCTACACAAAAGCTTTCCCATTCAACA
AAACCTTCAGGAAGCCCTTTGGAAGAAAAACACGATCAATGCAAATGTGAAAACCTTATAATGTTCCAGAA
CCTTGCAAACGAAGAAGTAAGAAAATTAACACAGCCCTTAGAAGAAATGACACAGAGAATGGAAGCCCTGG
AAAATCGCCTGAGATACAGATGAAGATTAGAAATCGCGACACATTTGTAGTCATTGTATCACGGATTACAA
TGAACGCAGTGCAGAGCCCCAAAGCTCAGGCTATTGTTAAATCAATAATGTTGTGAAGTAAAACAATCAGT
ACTGAGAAACCTGGTTTGCCACAGAACAAAGACAAGAAGTATACACTAACTTGTATAAATTTATCTAGGAA
AAAAATCCTTCAGAATTCTAAGATGAATTTACCAGGTGAGAATGAATAAGCTATGCAAGGTATTTTGTAAT
ATACTGTGGACACAACTTGCTTCTGCCTCATCCTGCCTTAGTGTGCAATCTCATTTGACTATACGATAAAG
TTTCCACAGTCTTACTTCTGTAGAACACTGGCCATAGGAAATGCTGTTTTTTTGTACTGGACTTTACCTTG
ATATATGTATATGGATGTATGCATAAAATCATAGGACATATGTACTTGTGGAACAAGTTGGATTTTTTATA
CAATATTAAAATTCACCACTTCAGAGAAAAGTAAAAAAA
NOV13h,
13374217 SNP
for CG51018-01 SEQ ID NO: 160 959 aa SNP: no change in the
Protein Sequence protein sequence
MEKMLAGCFLLILGQIVLLPAEARQRSRGRSISRGRHARTHPQTALLESSCENKRADLVFIIDSSRSVNTH
DYAKVKEFIVDILQFLDIGPDVTRVGLLQYGSTVKNEFSLKTFSLKSEVERAVKRMRHLSTGTMTGLAIQY
ALNIAFSEAEGARPLRENVPRVIMIVTDGRPQDSVAEVAAKARDTGILIFAIGVGQVDFNTLKSIGSEPHE
DHVFLVANFSQIETLTSVFQKKLCTAHMCSTLEHNCAHFCINIPGSYVCRCKQGYILNSDQTTCRIQDLCA
MEDHNCEQLCVNVPGSFVCQCYSGYALAEDGKRCVAVDYCASENHGCEHECVNADGSYLCQCHEGFALNPD
EKTCTKIDYCASSNHGCQYECVNTDDSYSCHCLKGFTLNPDKKTCRRINYCALNKPGCEHECVNMEESYYC
RCHRGYTLDPNGKPCSRVDHCAQQDHGCEQLCLNTEDSFVCQCSEGFLINEDLKTCSRVDYCLLSDHGCEY
SCVNMDRSFACQCPEGHVLRSDGKTCAKLDSCALGDHGCEHSCVSSEDSFVCQCFEGYILREDGKTCRRKD
VCQAIDHGCEHICVNSDDSYTCECLEGFRLTEDGKRCRISSGKDVCKSTHHGCEHICVNNGNSYICKCSEG
FVLAEDGRRCKKCTEGPIDLVFVIDGSKSLGEENFEVVKQFVTGIIDSLTISPKAARVGLLQYSTQVHTEF
TLRNFNSAKDMKKAVAHMKYMGKGSMTGLALKHMFERSFTQGEGARPFSTRVPRAAIVFTDGRAQDDVSEW
ASKAKANGITMYAVGVGKAIEEELQEIASEPTNKHLFYAEDFSTMDEISEKLKKGICEALEDSDGRQDSPA
GELPKTVQQPTESEPVTINIQDLLSCSNFAVQHRYLFEEDNLLRSTQKLSHSTKPSGSPLEEKHDQCKCEN
LIMFQNLANEEVRKLTQRLEEMTQRMEALENRLRYR

A ClustalW comparison of the above protein sequences yields the following sequence alignment shown in Table 13B.

TABLE 13B
Comparison of the NOV13 protein sequences.

| | |
|---|---|
| NOV13a | -----MEKMLAGCFLLILGQIVLLPAEARQRSRGRSISRGRHARTHPQTALLESSCENKR |
| NOV13b | ------------------------------------------------------------ |
| NOV13c | TRSPTMEKMLAGCFLLILGQIVLLPAEARERSRGRSISRGRHARTHPQTALLESSCENKR |
| NOV13d | TRSPTMEKMLAGCFLLILGQIVLLPAEARERSRGRSISRGRHARTHPQTALLESSCENKR |
| NOV13e | TRSPTMEKMLAGCFLLILGQIVLLPAEARERSRGRSISRGRHARTHPQTALLESSCENKR |
| NOV13f | ----------------------------RQRSRGRSISRGRHARTHPQTALLESSCENKR |
| NOV13g | -----MEKMLAGCFLLILGQIVLLPAEARERSRGRSISRGRHARTHPQTALLESSCENMR |
| NOV13a | ADLVFIIDSSRSVNTHDYAKVKEFIVDILQFLDIGPDVTRVGLLQYGSTVKNEFSLKTFK |
| NOV13b | ------------------------------------------------------------ |
| NOV13c | ADLVFIIDSSRSVNTHDYAKVKEFIVDILQFLDIGPDVTRVGLLQYGSTVKNEFSLKTFK |
| NOV13d | ADLVFIIDSSRSVNTHDYAKVKEFIVDILQFLDIGPDVTRVGLLQYGSTVKNEFSLKTFK |
| NOV13e | ADLVFIIDSSRSVNTHDYAKVKEFIVDILQFLDIGPDVTRVGLLQYGSTVKNEFSLKTFK |
| NOV13f | ADLVFIIDSSRSVNTHDYAKVKEFIVDILQFLDIGPDVTRVGLLQYGSTVKNEFSLKTFK |
| NOV13g | ADLVFIIDSSRSVNTNDYAKVKEFIVDILQFLDIGPDVTRVGLLQYCSTVKNEFSLKTFK |
| NOV13a | RKSEVERAVKRMRHLSTGTMTGLAIQYALNIAFSEAEGARPLRENVPRVIMIVTDGRPQD |
| NOV13b | ------------------------------------------------------------ |
| NOV13c | RKSEVERAVKMRHLSTGTMTGLAIQYALNIAFSEAEGARPLRENVPRVIMIVTDGQRPQD |
| NOV13d | RKSEVERAVKRMRHLSTGTMTGLAIQYALNIAFSEAEGARPLRENVPRVIMIVTDGRPQD |
| NOV13e | RKSEVERAVKRMRHLSTGTMTGLAIQYALNIAFSEAEGARPLRENVPRVIMIVTDGRPQD |
| NOV13f | RKSEVERAVKRMRHLSTGTMTGLAIQYALNIAFSEAEGARPLRENVPRVIMIVTDGRPQD |
| NOV13g | RKSEVERAVKRMRHLSTGTMTGLAIQYALNIAFSEAEGARPLRENVPRVIMIVTDGRPQD |
| NOV13a | SVAEVAAKARDTGILIFAIGVGQVDFNTLKSIGSEPHEDHVFLVANFSQIETLTSVFQKK |
| NOV13b | ------------------------------------------------------------ |
| NOV13c | SVAEVAAKARDTGILIFAIGVGQVDFNTLKSIGSEPHEDHVFLVANFSQIETLTSVFQKK |
| NOV13d | SVAEVAAKARDTGILIFAIGVGQVDFNTLKSIGSEPHEDHVFLVANFSQIETLTSVFQKK |
| NOV13e | SVAEVAAKARDTGILIFAIGVGQVDFNTLKSIGSEPHEDHVFLVANFSQIETLTSVFQKK |
| NOV13f | SVAEVAAKARDTGILIFAIGVGQVDFNTLKSIGSEPHEDHVFLVANFSQIETLTSVFQKK |
| NOV13g | SVAEVAAKARDTGILIFAIGVGQVDFNTLKSIGSEPHEDHVFLVANFSQIETLTSVFQKK |
| NOV13a | LCTAHMCSTLEHNCAHFCINIPGSYVCRCKQGYILNSDQTTCRIQDLCAMEDHNCEQLCV |
| NOV13b | ------------------------------------------------------------ |
| NOV13c | LCTAHMCSTLEHNCAHFCINIPGSYVCRCKQGYILNSDQTTCRIQDLCAMEDHNCEQLCV |
| NOV13d | LCTABMCSTLEHNCAHFCINIPGSYVCRCKQGYILNSDQTTCRIQDLCAMEDNNCEQLCV |
| NOV13e | LCTAHMCSTLEHNCAHFCINIPGSYVCRCKQGYILNSDQTTCRIQDLCAMEDHNCEQLCV |
| NOV13f | LCTAHMCSTLEHNCAHFCINIPGSYVCRCKQGYILNSDQTTCRIQDLCAMEDHNCEQLCV |
| NOV13g | LCTAHMCSTLEHNCAHFCINIPGSYVCRCKQGYILNSDQTTCRIQDLCAMEDHNCEQLCV |
| NOV13a | NVPGSFVOQCYSGYALAEDGKRCVAVDYCASENHGCEHECVNADGSYLCQCHEOFALNPD |
| NOV13b | ------------------------------------------------------------ |
| NOV13c | NVPGSFVCQCYSGYALAEDGKRCVAVDYCASENHGCENECVNADGSYLCQCHEGFALNPD |
| NOV13d | NVPGSFVCQCYSGYALAEDGKRCVAVDYCASENHGCENECVNADGSYLCQCHEGFALNPD |
| NOV13e | NVPGSFVCQCYSGYALAEDGKRCVAVDYCASENHOCENECVNADGSYLCQCHEGFALNPD |
| NOV13f | NVPGSFVCQCYSGYALAEDOKRCVAVDYCASENHGCENECVNADGSYLCQCNEGFALNPD |
| NOV13g | NVPGSFVCQCYSGYALAEDGKRCVAVDYCASS---------------------------- |
| NOV13a | EKTCTKIDYCASSNHGCQYECVNTDDSYSCHCLKGFTLNPDKKTCRRINYCALNKPGCEH |
| NOV13b | ------------------------------------------------------------ |
| NOV13c | EKTCTKIDYCASSNHGCQNECVNTDDSYSCHCLKGFTLNPDKKTCRRINYCALNKPGCEH |
| NOV13d | EKTCTKIDYCASSNHGCQHECVNTDDSYSCHCLKGFTLNPDKKTCRRINYCALNKPGCEH |
| NOV13e | EKTCTKIDYCASSNHGCQHECVNTDDSYSCHCLKGFTLNPDKKTCRRINYCALNKPGCEH |
| NOV13f | KKTCTKIDYCASSNHGCQHECVNTDDSYSCHCLKGFTLNPDKKACRRINYCALNKPGCEN |
| NOV13g | -------------NHGCQHECVNTDDSYSCHCLKGFTLNPDKKTCRRINYCALNKPGCEN |
| NOV13a | ECVNMEESYYCRCHRGYTLDPNGKP-CSRVDHCAQQDHGCEQLCLNTEDSFVCQCSEGFL |
| NOV13b | ALEGFSESHLA-LH--LAVNP-GKNFCHFGVH--TRFVSLATACAPAGD--TCQVPAVVL |
| NOV13c | ECVNMEESYYCRCHRGYTLDPNGKT-CSRVDHCAQQDHGCEQLCLNTEDSFVCQCSEGFL |
| NOV13d | ECVNMEESYYCRCHRGYTLDPNGKT-CSRVDHCAQQDHGCEQLCLNTEDSFVCQCSEOFL |
| NOV13e | ECVNMEESYYCRCNRGYTLDPNGKT-CSRVDHCAQQDHGCEQLCLNTEDSFVCQCSEOFL |
| NOV13f | ECVNMEESYYCRCHRGYTLDPNGKT-CSRVDHCAQQDHGCEQLCLNTEDSFVCQCSEOFL |
| NOV13g | ECVNMEESYYCRCHRGYTLDPNGKT-CSRVDNCAQQDHGCEQLCLNTEDSFVCQCSEGFL |
| NOV13a | INEDLKTCSRVDYCLLSDHGCEYSCVNMDRSFACQCPEGHVLRSDGKTCAKLDSCALGDH |
| NOV13b | LTDKRP--PAVSLAGVSAP------TKVPSTHH---PWGKVTVIDQVVIAFLEVDEIDLH |
| NOV13c | INEDLKTCSRVDYCLLSDNGCEYSCVNMDRSFACQCPEGHVLRSOGKTCAKLDSCALGDH |
| NOV13d | INEDLKTCSRVDYCLLSDHGCEYSCVNMDRSFACQCPEGHVLRSDGKTCAKLDSCALGDN |
| NOV13e | INEDLKTCSRVDYCLLSDHOCEYSCVNMDRSFACQCPEGHVLRSDGKTCAKLDSCALGDH |
| NOV13f | INEDLKTCSRVDYCLLSDHGCEYSCVNMDRSFACQCPEGHVLRSDGKTCAKLDSCALGDH |
| NOV13g | INEDLKTCSRVDYCLLSDHGCEYSCVNMDRSFACQCPEGHVLRSDGKTCAKLDSCALGDH |
| NOV13a | G-CEHSCVSSEDSFVCQCFEGYILREDGKTCRRKOVCQAIDHGCENICVNSDDSYTCECL |
| NOV13b | LPEEGGCLVI-------CLPGLA-----KACDPAGLIEAKGLS-MHGEASRVD--VSGQG |
| NOV13C | G-CEHSCVSSEDSFVCQCFEGYILREDGKTCRRKDVCQAIDHGCEHICVNSDDSYTCECL |
| NOV13d | G-CEHSCVSSEDSFVCQCFEGYILREDGKTCRRKDVCQAIDHGCEHICVNSDDSYTCECL |
| NOV13e | G-CEHSCVSSEDSFVCQCFEGYILREDGKTCRRKDVCQAIDHGCEHICVNSDDSYTCECL |
| NOV13f | G-CEHSCVSSEDSFVCQCFEGYILREDGKTCRRKDVCQAIDHGCEHICVNSDDSYTCECL |
| NOV13g | G-CEHSCVSSEDSFVCQCFEGYILREDGKTCRRKDVCQAIDNGCEHICVNSDDSYTCECL |
| NOV13a | EGFRLTEDGKRCRISSGKDVCKSTHHGCEHICVNNGNSYICKCSEGFVLAEDGRRCKKCT |
| NOV13b | QGLGQPHEG---------DVIVVLLIGVIAVDDDLGNG---GCLRQLVQVA-GAR----V |
| NOV13c | EGFRLAEDGKRCRR---KDVCKSTHHGCEHICVNNGNSYICKCSEGFVLAEDGRRCKKCT |
| NOV13d | EGFRLAEDGKRCRR---KDVCKSTHHGCEHICVNNGNSYICKCSEGFVLAEDGRRCKKCT |
| NOV13e | EGFRLAEDGKRCRR---KDVCKSTHHGCENICVNNGNSYICKCSEGFVLAEDGRRCKKCT |
| NOV13f | EGFRLAEDOKRCRR---KDVCKSTHHGCEHICVNNGNSYICKCSEGFVLAEDGRRCKKCT |
| NOV13g | EGFRLAEDGKRCRR---KDVCKSTHHGCEHICVNNGNSYICKCSEGFVLAEDGRRCKKCT |
| NOV13a | EGPIDLVFVIDGSKSLGEENFEVVKQFVTGIIDSLTISPKA-ARVGLLQYSTQVHTEFTL |
| NOV13b | HLPA--LQDLLP----GRE--EAVG----GSEHPLGVNERASTDVGGAEV--QTHLPRPL |
| NOV13c | EGPIDLVFVIDGSKSLGEENFEVVKQFVTGIIDSLTISPKA-ARVGLLQYSTQVHTEFTL |
| NOV13d | EGPIDLVFVIDGSKSLGEENFEVVKQFVTGIIDSLTISPKA-ARVGLLQYSTQVHTEFTL |
| NOV13e | EGPIDLVFVIDGSKSLGEENFEVVKQFVTGIIDSLTISPKA-ARVGLLQYSTQVHTEFTL |
| NOV13f | EGPIDLVFVIDGSKSLGEENFEVVKQFVTGIIDSLTISPKA-ARVGLLQYSTQVHTEFTL |
| NOV13g | EGPIDLVFVTDGSKSLGEENFEVVKQFVTGIIDSLTISPKA-ARVGLLQYSTQVHTEFTL |
| NOV13a | RNFNSAKDMKKAVAHMKYMGKGSMTGLALKHMFERSFTQGEGARPFSTRVPRAAIVFTDG |
| NOV13b | AIRGQRP------SHDP---PG--------HOPQSAVGTLEGDIPLG-RAFRPVEAFLDG |
| NOV13c | RNFNSAKDMKKAVAHMKYMGKGSMTGLALKHMFERSFTQGEGARPLSTRVPRAAIVFTDG |
| NOV13d | RNFNSAKDMKKAVAHMKYMGKGSMTGLALKHMFERSFTQGEGARPLSTRVPRAAIVFTDG |
| NOV13e | RNFNSAKDMKKAVAHMKYMGKGSMTGLALKHMFERSFTQGEGARPLSTRVPRAAIVFTDG |
| NOV13f | RNFNSAKDMKKAVAHMKYMGKGSMTGLALKHIFERSFTQGEGARPLSTRVPRAAIVFTDG |
| NOV13g | RNFNSAKDMKKAVAHMKYMGKGSMTGLALKHMFERSFTQGEGARPLSTRVPRAAIVFTDG |
| NOV13a | --RAQDDVSEWASKAKANGITMYAVGVGKAIEEELQEIASEPTNKHLFYAEDFSTMDEIS |
| NOV13b | GVVSQD------------------------------------------------------ |
| NOV13c | --RAQDDVSEWASKAKANGITMYAVGVGKAIEEELQEIASEPTNKHLFYAEDFSTMDEIS |
| NOV13d | --RAQDDVSEWASKAKANGITMYAVGVGKAIEEELQEIASEPTNKHLFYAEDFSTMDEIS |
| NOV13e | --RAQDDVSEWASKAKANGITMYAVGVGKAIEEELQEIASEPTNKHLFYAEDFSTMDEIS |
| NOV13f | --RAQDDVSEWASKAKANCITMYAVGVGKAIEEELQEIASEPTNKHLFYAEDFSTMDEIS |
| NOV13g | --RAQDDVSEWASKAKANGITMYAVGVGKAIEEELQEIASEPTNKHLFYAEDFSTMDEIS |
| NOV13a | EKLKKGICEALEDSDGRQDSPAGELPKTVQQPTESEPVTINIQDLLSCSNFAVQHRYLFE |
| NOV13b | ------------------------------------------------------------ |
| NOV13c | EKLKKGICEALEDSDGRQDSPAGELPKTVQQPTESEPVTINIQDLLSCSNFAVQHRYLFE |
| NOV13d | EKLKKGICEALEDSDGRQDSPAGELPKTVQQPTESEPVTINIQDLLSCSNFAVQHRYLFE |
| NOV13e | EKLKKGICEALEDSDGRQDSPAGELPKTVQQPT-------------------VQHRYLFE |
| NOV13f | EKLKKGICEALEDSDGRQDSPAGELPKTVQQPT-------------------VQNRYLFE |
| NOV13g | EKLKKGICEALEDSDGRQDSPAGELPKTVQQPT-------------------VQHRYLFE |
| NOV13a | EDNLLRSTQKLSHSTKPSGSPLEEKHDQCKCENLIMFQNLANEEVRKLTQRLEEMTQRME |
| NOV13b | ------------------------------------------------------------ |
| NOV13c | EDNLLRSTQKLSHSTKPSGSPLEEKHDQCKCENLIMFQNLANEEVRKLTQRLEEMTQRME |
| NOV13d | EDNLLRSTQKLSHSTKPSGSPLEEKHDQCKCENLIMFQNLANEEVRKLTQRLEEMTQRME |
| NOV13e | EDNLLRSTQKLSHSTKPSGSPLEEKHDQCKCENLIMFQNLANEEVRKLTQRLEEMTQRME |
| NOV13f | EDNLLRSTQKLSHSTKPSGSPLEEKHDQCKCENLIMFQNLANEEVRKLTQRLEEMTQRME |
| NOV13g | EDNLLRSTQKLSHSTKPSGSPLEEKHDQCKCENLIMFQNLANEEVRKLTQRLEEMTQRME |
| NOV13a | ALENRLRYR--- |
| NOV13b | ------------ |
| NOV13c | ALENRLRYRVDG |
| NOV13d | ALENRLRYRVDG |
| NOV13e | ALENRLRYRVDG |
| NOV13f | ALENRLRYR--- |
| NOV13g | ALENRLRYR--- |
| NOV13a | (SEQ ID NO: 146) |
| NOV13b | (SEQ ID NO: 148) |
| NOV13c | (SEQ ID NO: 150) |
| NOV13d | (SEQ ID NO: 152) |
| NOV13e | (SEQ ID NO: 154) |
| NOV13f | (SEQ ID NO: 156) |
| NOV13g | (SEQ ID NO: 158) |

Further analysis of the NOV13a protein yielded the following properties shown in Table 13C.

TABLE 13C
Protein Sequence Properties NOV13a

| SignalP | Cleavage site between residues 24 and 25 | | | |
|---|---|---|---|---|
| analysis: | | | | |
| PSORT II | PSG: a new signal peptide prediction method | | | |
| analysis: | N-region: length 3; pos. chg 1; neg. chg 1 | | | |
| | H-region: length 18; peak value 10.51 | | | |
| | PSG score: 6.11 | | | |
| | GvH: von Heijne's method for signal seq. recognition | | | |
| | GvH score (threshold: −2.1): −0.88 | | | |
| | possible cleavage site: between 23 and 24 | | | |
| | >>> Seems to have a cleavable signal peptide (1 to 23) | | | |
| | ALOM: Klein et al's method for TM region allocation | | | |
| | Init position for calculation: 24 | | | |
| | Tentative number of TMS(s) for the threshold 0.5: 0 | | | |
| | number of TMS(s) . . . fixed | | | |
| | PERIPHERAL Likelihood = 1.27 (at 188) | | | |
| | ALOM score: 1.27 (number of TMSs: 0) | | | |
| | MTOP: Prediction of membrane topology (Hartmann et al.) | | | |
| | Center position for calculation: 11 | | | |
| | Charge difference: 4.0 C(5.0)-N(1.0) | | | |
| | C > N: C-terminal side will be inside | | | |
| | >>>Caution: Inconsistent mtop result with signal peptide | | | |
| | MITDISC: discrimination of mitochondrial targeting seq | | | |
| | R content: | 0 | Hyd Moment(75): | 8.86 |
| | Hyd Moment(95): | 9.72 | G content: | 2 |
| | D/E content: | 2 | S/T content: | 0 |

| Score: −7.20 | |
|---|---|
| Gavel: prediction of cleavage sites for mitochondrial preseq | |
| R-2 motif at 49 ART\|HP | |
| NUCDISC: discrimination of nuclear localization signals | |
| pat4: none | |
| pat7: none | |
| bipartite: none | |
| content of basic residues: 11.4% | |
| NLS Score: −0.47 | |
| KDEL: ER retention motif in the C-terminus: none | |
| ER Membrane Retention Signals: none | |
| SKL: peroxisomal targeting signal in the C-terminus: none | |
| PTS2: 2nd peroxisomal targeting signal: none | |
| VAC: possible vacuolar targeting motif: none | |
| RNA-binding motif: none | |
| Actinin-type actin-binding motif: | |
| type 1: none | |
| type 2: none | |
| NMYR: N-myristoylation pattern: none | |
| Prenylation motif: none | |
| memYQRL: transport motif from cell surface to Golgi: none | |
| Tyrosines in the tail: none | |
| Dileucine motif in the tail: none | |
| checking 63 PROSITE DNA binding motifs: none | |
| checking 71 PROSITE ribosomal protein motifs: none | |
| checking 33 PROSITE prokaryotic DNA binding motifs: none | |
| NNCN: Reinhardt's method for Cytoplasmic/Nuclear discrimination | |
| Prediction: nuclear | |
| Reliability: 76.7 | |
| COIL: Lupas's algorithm to detect coiled-coil regions | |
| 925 M | 0.82 |
| 926 F | 0.94 |
| 927 Q | 1.00 |
| 928 N | 1.00 |
| 929 L | 1.00 |
| 930 A | 1.00 |
| 931 N | 1.00 |
| 932 E | 1.00 |
| 933 E | 1.00 |
| 934 V | 1.00 |
| 935 R | 1.00 |
| 936 K | 1.00 |
| 937 L | 1.00 |
| 938 T | 1.00 |
| 939 Q | 1.00 |
| 940 R | 1.00 |
| 941 L | 1.00 |
| 942 E | 1.00 |
| 943 E | 1.00 |
| 944 M | 1.00 |
| 945 T | 1.00 |
| 946 Q | 1.00 |
| 947 R | 1.00 |
| 948 M | 1.00 |
| 949 E | 1.00 |
| 950 A | 1.00 |
| 951 L | 1.00 |
| 952 E | 1.00 |
| 953 N | 1.00 |
| 954 R | 1.00 |
| 955 L | 1.00 |
| 956 R | 1.00 |
| 957 Y | 1.00 |
| 958 R | 0.99 |

```
total: 34 residues
Final Results (k = 9/23):
60.9%: nuclear
17.4%: mitochondrial
  8.7%: cytoplasmic
  8.7%: extracellular, including cell wall
  4.3%: peroxisomal
>> prediction for CG51018-01 is nuc (k = 23)
```

A search of the NOV13a protein against the Geneseq database, a proprietary database that contains sequences published in patents and patent publication, yielded several homologous proteins shown in Table 13D.

TABLE 13D
Geneseq Results for NOV13a

| | | | Identities/ | |
|---|---|---|---|---|
| | | | Similarities for | |
| Geneseq | Protein/Organism/Length | NOV13a Residues/ | the Matched | Expect |
| Identifier | [Patent #, Date] | Match Residues | Region | Value |
| ABJ10549 | Human NOV4a protein - Homo | 1 . . . 959 | 959/959 (100%) | 0.0 |
| | sapiens, 977 aa. [WO200246408-A2, | 19 . . . 977 | 959/959 (100%) | |
| | 13 JUN. 2002] | | | |
| AAB20159 | Human protein SECP5 - Homo | 1 . . . 959 | 956/959 (99%) | 0.0 |
| | sapiens, 959 aa. [WO200105971-A2, | 1 . . . 959 | 958/959 (99%) | |
| | 25 JAN. 2001] | | | |
| AAE03877 | Human gene 3 encoded secreted | 1 . . . 959 | 929/959 (96%) | 0.0 |
| | protein fragment, SEQ ID NO: 127 - | 47 . . . 983 | 931/959 (96%) | |
| | Homo sapiens, 983 aa. | | | |
| | [WO200136440-A1, 25 MAY 2001] | | | |
| ABG64542 | Human albumin fusion protein #1217 - | 4 . . . 9S9 | 926/956 (96%) | 0.0 |
| | Homo sapiens, 934 aa. | 1 . . . 934 | 928/956 (96%) | |
| | [WO200177137-A1, 18 OCT. 2001] | | | |
| AAE03843 | Human gene 3 encoded secreted | 4 . . . 959 | 926/956 (96%) | 0.0 |
| | protein HOGDP46, SEQ ID NO: 89 - | 1 . . . 934 | 928/956 (96%) | |
| | Homo sapiens, 934 aa. | | | |
| | [WO200136440-A1, 25 MAY 2001] | | | |

In a BLAST search of public sequence databases, the NOV13a protein was found to have homology to the proteins shown in the BLASTP data in Table 13E.

TABLE 13E
BLASTP Results for NOV13a

| | | NOV13a | Identities/ | |
|---|---|---|---|---|
| Protein | | Residues/ | Similarities for | |
| Accession | | Match | the Matched | |
| Number | Protein/Organism/Length | Residues | Portion | Expect Value |
| CAC32426 | Sequence 9 from Patent | 1 . . . 959 | 956/959 (99%) | 0.0 |
| | WO0105971 - Homo sapiens | 1 . . . 959 | 958/959 (99%) | |
| | (Human), 959 aa. | | | |
| Q8NDE6 | Hypothetical protein - Homo | 1 . . . 959 | 950/959 (99%) | 0.0 |
| | sapiens (Human), 1016 aa | 61 . . . 1016 | 952/959 (99%) | |
| | (fragment). | | | |
| O00339 | Matrilin-2 precursor - Homo | 1 . . . 959 | 949/959 (98%) | 0.0 |
| | sapiens (Human), 956 aa. | 1 . . . 956 | 952/959 (98%) | |
| Q96FT5 | Matrilin 2 - Homo sapiens | 1 . . . 959 | 931/959 (97%) | 0.0 |
| | (Human), 937 aa. | 1 . . . 937 | 933/959 (97%) | |
| Q99K64 | Matrilin 2 - Mus musculus | 1 . . . 959 | 832/960 (86%) | 0.0 |
| | (Mouse), 956 aa. | 1 . . . 956 | 888/960 (91%) | |

PFam analysis predicts that the NOV13a protein contains the domains shown in the Table 13F.

TABLE 13F
Domain Analysis of NOV13a

| | | Identities/ | |
|---|---|---|---|
| | | Similarities for | |
| Pfam | NOV13a Match | the Matched | Expect |
| Domain | Region | Portion | Value |
| vwa | 57 . . . 232 | 80/197 (41%) | 4.3e−64 |
| | | 161/197 (82%) | |
| EGF | 242 . . . 277 | 13/47 (28%) | 1.2e−06 |
| | | 27/47 (57%) | |
| EGF | 283 . . . 318 | 17/47 (36%) | 9.1e−09 |
| | | 30/47 (64%) | |
| EGF | 324 . . . 359 | 15/47 (32%) | 8.8e−08 |
| | | 30/47 (64%) | |
| EGF | 365 . . . 400 | 15/47 (32%) | 7.7e−06 |
| | | 28/47 (60%) | |
| EGF | 406 . . . 441 | 16/47 (34%) | 6.2e−06 |
| | | 29/47 (62%) | |
| EGF | 447 . . . 482 | 11/47 (23%) | 1.1e−05 |
| | | 28/47 (60%) | |
| EGF | 488 . . . 523 | 13/47 (28%) | 2.9e−06 |
| | | 26/47 (55%) | |
| granulin | 488 . . . 523 | 11/44 (25%) | 0.82 |
| | | 22/44 (50%) | |
| TIL | 476 . . . 529 | 15/73 (21%) | 0.8 |
| | | 39/73 (53%) | |
| EGF | 529 . . . 564 | 13/47 (28%) | 2.1e−06 |
| | | 29/47 (62%) | |
| EGF | 570 . . . 605 | 16/47 (34%) | 2.4e−07 |
| | | 29/47 (62%) | |
| EGF | 614 . . . 649 | 13/47 (28%) | 4.8e−08 |
| | | 29/47 (62%) | |
| vwa | 658 . . . 833 | 83/197 (42%) | 1.6e−63 |
| | | 156/197 (79%) | |

### Example 14

The NOV14 clone was analyzed, and the nucleotide and encoded polypeptide sequences are shown in Table 14A.

TABLE 14A
NOV14 Sequence Analysis

NOV14a,
CG51051-07 SEQ ID NO: 161 1495bp
DNA Sequence ORF Start: ATG at 46 ORF Stop: TAG at 1486
TCAAGCTCTGCTTTAGTTTCCAAGAAGATTACAAAGAATTTAGAGATGTATTTGTCAAGATTCCTGTC
GATTCATGCCCTTTGGGTTACGGTGTCCTCAGTGATGCAGCCCTACCCTTTGGTTTGGGGACATTATG
ATTTGTGTAAGACTCAGATTTACACGGAAGAAGGGAAAGTTTGGGATTACATGGCCTGCCAGCCGGAA
TCCACGGACATGACAAAATATCTGAAAGTGAAACTCGATCCTCCGGATATTACCTGTGGAGACCCTCC
TGAGACGTTCTGTGCAATGGGCAATCCCTACATGTGCAATAATGAGTGTGATGCGAGTACCCCTGAGC
TGCCACACCCCCCTGAGCTGATGTTTGATTTTGAAGGAAGACATCCCTCCACATTTTGGCAGTCTGCC
ACTTGGAAGGAGTATCCCAAGCCTCTCCAGGTTAACATCACTCTGTCTTGGAGCAAAACCATTGAGCT
AACAGACAACATAGTTATTACCTTTGAATCTGGGCGTCCAGACCAAATGATCCTGGAGAAGTCTCTCG
ATTATGGACGAACATGGCAGCCCTATCAGTATTATGCCACAGACTGCTTAGATCCTTTTCACATGGAT
CCTAAATCCGTGAAGGATTTATCACAGCATACGGTCTTAGAAATCATTTGCACAGAAGAGTACTCAAC
AGGGTATACAACAAATAGCAAAATAATCCACTTTGAAATCAAAGACAGGTTCGCGTTTTTTGCTGGAC
CTCGCCTACGCAATATGGCTTCCCTCTACGGACAGCTGGATACAACCAAGAAACTCAGAGATTTCTTT
ACAGTCACACACCTGAGGATAACGCTGTTAAGACCAGCCGTTGGGGAAATATTTGTAGATGAGCTACA
CTTGGCACGCTACTTTTACGCGATCTCAGACATAAAGGTGCGAGGAAGGTGCAAGTGTAATCTCCATG
CCACTGTATGTGTGTATGACAACAGCAAATTGACATGCGAATGTGAGCACAACACTACAGGTCCAGAC
TGTGGGAAATGCAAGAAGAATTATCAGGGCCGACCTTGGAGTCCAGGCTCCTATCTCCCCATCCCCAA
AGGCACTGCAAATACCTGTATCCCCAGTATTTCCAGTATTGGTAATCCTCCAAAGTTTAATAGGATAT
GGCCGAATATTTCTTCCCTTGAGGTTTCTAACCCAAAACAAGTTGCTCCCAAATTAGCTTTGTCAACA
GTTTCTTCTGTTCAAGTTGCAAACCACAAGAGAGCGAATGTCTGCCACAACGAGCTCCTGCACTGCCA
GAACGGACGGACGTGCCACAACAACGTGCGCTGCCTGTGCCCGGCCGCATACACGGGCATCCTCTGCG
AGAAGCTGCGGTGCGAGGAGGCTGGCAGCTGCGGCTCCGACTCTGGCCAGGGCGCGCCCCCGCACGGC
TCCCCAGCGCTGCTGCTGCTGACCACGCTGCTGGGAACCGCCAGCCCCCTGGTGTTCTAGGTGTCAC
NOV14a,
CG51051-07
Protein Sequence SEQ ID NO: 162 480 aa Mw at 53945.0 kD
MYLSRFLSIHALWVTVSSVMQPYPLVWGHYDLCKTQIYTEEGKVWDYMACQPESTDMTKYLKVKLDPP
DITCGDPPETFCAMGNPYMCNNECDASTPELAHPPELMFDFEGRHPSTFWQSATWKEYPKPLQVNITL
SWSKTIELTDNIWETFESGRPDQMILEKSLDYGRTWQPYQYYATDCLDAFHMDPKSVKDLSQHTVLEI
ICTEEYSTGYTTNSKIIHFEIKDRFAFFAGPRLRNMASLYGQLDTTKKLRDFFTVTDLRIRLLRPAVG
EIFVPELHLARYFYAISDIKVRGRCKCNLHATVCVYDNSKLTCECEHNTTGPDCGKCKKNYQGRPWSP
GSYLPIPKGTANTCIPSISSIGNPPKFNRIWPNISSLEVSNPKQVAPKLALSTVSSVQVANHKRANVC
DNELLHCQNGGTCHNNVRCLCPAAYTGILCEKLRCEEAGSCGSDSGQGAPPHGSPALLLLTTLLGTAS
PLVF
NOV14b,
CG51051-14 SEQ ID NO: 163 1369 bp
DNA Sequence ORF Start: ATG at 46 ORF Stop: TAG at 1360
TCAAGCTCTGCTTTAGTTTCCAAGAAGATTACAAAGAATTTAGAGATGTATTTGTCAAGATTCCTGTC
GATTCATGCCCTTTGGGTTACGGTGTCCTCAGTGATGCAGCCCTACCCTTTGGTTTGGGGACATTATG
ATTTGTGTAAGACTCAGATTTACACGGAAGAAGGGAAAGTTTGGGATTACATGGCCTGCCAGCCGGAA
TCCACGGACATGACAAAATATCTGAAAGTGAAACTCGATCCTCCGGATATTACCTGTGGAGACCCTCC
TGAGACGTTCTGTGCAATGGGCAATCCCTACATGTGCAATAATGAGTGTGATGCGAGTACCCCTGAGC
TGGCACACCCCCCTGAGCTGATGTTTGATTTTGAAGGAAGACATCCCTCCACATTTTGGCAGTCTGCC
ACTTCGAAGGAGTATCCCAAGCCTCTCCAGGTTAACATCACTCTGTCTTGGAGCAAAACCATTGAGCT
AACAGACAACATAGTTATTACCTTTGAATCTGGGCGTCCAGACCAAATGATCCTGGAGAAGTCTCTCG
ATTATGGACGAACATGGCAGCCCTATCAGTATTATGCCACAGACTGCTTAGATGCTTTTCACATGGAT
CCTAAATCCGTGAAGGATTTATCACAGCATACGGTCTTAGAAATCATTTGCACAGAAGAGTACTCAAC
AGGGTATACAACAAATAGCAAAATAATCCACTTTGAAATCAAAGACAGGTTCGCGTTTTTTGCTGGAC
CTCGCCTACGCAATATGGCTTCCCTCTACGGACAGCTGGATACAACCAAGAAACTCAGAGATTTCTTT
ACAGTCACAGACCTGAGGATAAGGCTGTTAAGACCAGCCGTTGGGGAAATATTTGTAGATGAGCTACA
CTTGGCACGCTACTTTTACGCGATCTCAGACATAAAGGTGCGAGGAAGGTGCAAGTGTAATCTCCATG
CCACTGTATGTGTGTATGACAACAGCAAATTGACATGCGAATGTGAGCACAACACTACAGGTCCAGAC
TCTGGGAAATGCAAGAAGAATTATCAGGGCCGACCTTGGAGTCCAGGCTCCTATCTCCCCATCCCCAA
AGGCACTCCAAATACCTGTATCCCCAGTATTTCCAGTATTGGTACGAATGTCTGCGACAACGAGCTCC
TGCACTGCCAGAACGGAGGGACGTGCCACAACAACGTGCGCTGCCTGTGCCCGGCCGCATACACGAGC
ATCCTCTGCGAGAAGCTGCGGTGCGAGGAGGCTCGCAGCTGCCGCTCCGACTCTGGCCAGGGCGCGCC
CCCGCACGGCTCCCCAGCCCTGCTGCTGCTCACCACGCTGCTGGGAACCGCCAGCCCCCTGGTGTTCT
AGGTGTCAC
NOV14b,
CG51051-14
Protein Sequence SEQ ID NO: 164 438 aa MW at 49339.7 kD
MYLSRFLSIHALWVTVSSVMQPYPLVWGHYDLCKTQIYTEEGKVWDYMACQPESTDMTKYLKVKLDPP
DITCGDPPETFCAMGNPYMCNNECDASTPELAHPPELMFDFEGRHPSTFWQSATWKEYPKPLQVNITL
SWSKTIELTDNIVITFESGRPDQMILEKSLDYGRTWQPYQYYATDCLDAFHMDPKSVKDLSQHTVLEI
ICTEEYSTGYTTNSKIIHFEIKDRFAFFAGPRLRNMASLYGQLDTTKKLRDFFTVTDLRIRLLRPAVG
EIFVDELHLARYFYAISDIKVRGRCKCNLHATVCVYDNSKLTCECEHNTTGPDCGKCKKNYQGRPWSP
GSYLPIPKGTANTCIPSISSIGTNVCDNELLHCQNGGTCHNNVRCLCPAAYTGILCEKLRCEEAGSCG
SDSGQGAPPHGSPALLLLTTLLGTASPLVF
NOV14c,
254537195 SEQ ID NO: 165 1398 bp
DNA Sequence ORF Start: at 1 ORF Stop: end of sequence
AGATCTGTGATGCAGCCCTACCCTTTGGTTTGUGGACATTATGATTTGTGTAAGACTCAGATTTACAC
GGAAGAAGGGAAAGTTTGGGATTACATGGCCTGCCAGCCGGAATCCACGGACATGACAAAATATCTGA
AAGTCAAACTCCATCCTCCGGATATTACCTGTGGAGACCCTCCTGAGACGTTCTGTGCAATCGGCAAT
CCCTACATGTCCAATAATGAGTGTGATGCGAGTACCCCTGAGCTGGCACACCCCCCTGAGCTGATGTT
TGATTTTGAAGGAAGACATCCCTCCACATTTTGGCAGTCTGCCACTTGGAAGGAGTATCCCAAGCCTC
TCCAGGTTAACATCACTCTGTCTTGGAGCAAAACCATTGAGCTAACAGACAACATAGTTATTACCTTT
GAATCTGGGCGTCCAGACCAAATGATCCTGGAGAAGTCTCTCGATTATGGACGAACATGGCAGCCCTA
TCAGTATTATGCCACAGACTGCTTAGATGCTTTTCACATGGATCCTAAATCCGTGAAGGATTTATCAC
AGCATACGGTCTTAGAATCATTTGCACAGAAGAGTACTCAACAGCGTATACAACAAATAGCAAAAATA
ATCCACTTTGAAATCAAACACAGGTTCGCGTTTTTTGCTGGACCTCGCCTACGCAATATGGCTTCCCT
CTACGGACAGCTGGATACAACCAAGAAACTCAGAGATTTCTTTACAGTCACAGACCTGAGGATAAGGC
TGTTAACACCAGCCGTTCGGGAAATATTTGTAGATGAGCTACACTTGGCACGCTACTTTTACGCGATC
TCAGACATAAAGGTGCGAGGAAGGTGCAAGTGTAATCTCCATGCCACTGTATGTGTGTATCACAACAG
CAAATTGACATGCGAATGTGAGCACAACACTACAGGTCCAGACTGTGGGAAATGCAAGAAGAATTATC
AGGGCCGACCTTGGAGTCCAGGCTCCTATCTCCCCATCCCCAAAGGCACTGCAAATACCTGTATCCCC
AGTATTTCCAGTATTGGTAATCCTCCAAAGTTTAATAGGATATGGCCGAATATTTCTTCCCTTGAGGT
TTCTAACCCAAAACAAGTTGCTCCCAAATTAGCTTTGTCAACAGTTTCTTCTGTTCAAGTTGCAAACC
ACAAGAGAGCGAATGTCTGCGACAACGAGCTCCTGCACTGCCAGAACGGAGGGACGTGCCACAACAAC
GTGCGCTGCCTGTGCCCGGCCGCATACACGGGCATCCTCTGCGAGAAGCTGCGGTGCGAGGAGGCTGG
CAGCTGCGGCTCCGACTCTGGCCAGGGCGCGCCCCCGCACGGCTCCCCAGCGCTGCTGCTGCTGACCA
CGCTGCTGGGAACCGCCAGCCCCCTGGTGTTCCTCGAG
NOV14c,
254537195
Protein Sequence SEQ ID NO: 166 466 aa MW at 52338.1 kD
RSAAQPYPLVWGHYDLCKTQIYTEEGKVWDYMACQPESTDMTKYLKVKLDPPDITCGDPPETFCAAGN
PYMCNNECDASTPELAHPPELMFDFEGRHPSTFWQSATWKEYPKPLQVNITLSWSKTIELTDNIVITF
ESGRPDQMILEKSLDYGRTWQPYQYYATDCLDAFHMDPKSVKDLSQHTVLEIICTEEYSTGYTTNSKI
IHFEIKDRFAFFAGPRLRNMASLYGQLDTTKKLRDFFTVTDLRIRLLRPAVGEIFVDELHLARYFYAI
SDIKVRGRCKCNLHATVCVYDNSKLTCECEHNTTGPDCGKCKKNYQGRPWSPGSYLPIPKGTANTCIP
SISSIGNPPKFNRIWPNISSLEVSNPKQVAPKLALSTVSSVQVANNKRANVCDNELLHCQNGGTCHNN
VRCLCPAAYTGILCEKLRCEEAGSCGSDSGQGAPPHGSPALLLLTTLLGTASPLVFLE
NOV14d,
254537282 SEQ ID NO: 167 750 bp
DNA Sequence ORF Start: at 1 ORF Stop: end of sequence
AGATCTTGCCAGCCGGAATCCACCGACATGACAAAATATCTGAAAGTGAAACTCGATCCTCCGGATAT
TACCTGTGGAGACCCTCCTGAGACGTTCTGTGCAATGGGCAATCCCTACATGTGCAATAATGAGTGTG
ATGCCAGTACCCCTGAGCTGGCACACCCCCCTGAGCTGATGTTTGATTTTGAAGGAAGACATCCCTCC
ACATTTTGGCAGTCTGCCACTTGGAAGGAGTATCCCAAGCCTCTCCAGGTTAACATCACTCTGTCTTG
GAGCAAAACCATTGAGCTAACAGACAACATAGTTATTACCTTTGAATCTGGGCGTCCAGACCAAATGA
TCCTGGAGAAGTCTCTCGATTATGGACGAACATGGCAGCCCTATCAGTATTATGCCACAGACTGCTTA
GATGCTTTTCACATGGATCCTAAATCCGTGAAGGATTTATCACAGCATACGGTCTTAGAAATCATTTG
CACAGAAGAGTACTCAACAGGGTATACAACAAATAGCAAAATAATCCACTTTGAAATCAAAGACAGGT
TCGCGTTTTTTGCTGGACCTCGCCTACGCAATATGGCTTCCCTCTACGGACAGCTGGATACAACCAAG
AAACTCAGAGATTTCTTTACAGTCACAGACCTGAGGATAAGGCTGTTAAGACCAGCCGTTGGGGAAAT
ATTTGTAGATGAGCTACACTTGGCACGCTACTTTTACGCGATCTCAGACATAAAGGTGCGAGGACTCG
AG
NOV14d,
254537282
Protein Sequence SEQ ID NO: 168 250 aa MW at 28958.6 kD
RSCQPESTDMTKYLKVKLDPPDITCGDPPETFCAMGNPYMCNNECDASTPELAHPPELMFDFEGRHPS
TFWQSATWKEYPKPLQVNITLSWSKTIELTDNIVITFESGRPDQMILEKSLDYGRTWQPYQYYATDCL
DAFHMDPKSVKDLSQHTVLETICTEEYSTGYTTNSKIIHFEIKDRFAFFAGPRLRNMASLYGQLDTTK
KLRDFFTVTDLRIRLLRPAVGEIFVDELHLARYFYAISDIKVRGLE
NOV14e,
CG51051-09 SEQ ID NO: 169 1480 bp
DNA Sequence ORF Start ATG at 26 ORF Stop: end of sequence
AGGCTCCGCGGCCGCCCCCTTCACCATGTATTTGTCAAGATTCCTGTCGATTCATGCCCTTTGGGTTA
CGGTGTCCTCAGTGATGCAGCCCTACCCTTTGGTTTGCGGACATTATGATTTGTGTAAGACTCAGATT
TACACCGAAGAAGGGAAAGTTTGGGATTACATGGCCTGCCAGCCGGAATCCACGGACATGACAAAATA
TCTGAAAGTGAAACTCGATCCTCCGGATATTACCTGTGGAGACCCTCCTGAGACGTTCTGTGCAATGG
GCAATCCCTACATGTGCAATAATGAGTGTGATGCGAGTACCCCTGAGCTGGCACACCCCCCTGAGCTG
ATGTTTGATTTTGAAGGAAGACATCCCTCCACATTTTCGCAGTCTGCCACTTGGAAGGAGTATCCCAA
GCCTCTCCAGGTTAACATCACTCTGTCTTGGAGCAAAACCATTGAGCTAACAGACAACATAGTTATTA
CCTTTGAATCTGGGCGTCCAGACCAAATGATCCTGGAGAAGTCTCTCGATTATGGACGAACATGGCAG
CCCTATCAGTATTATGCCACAGACTGCTTAGATGCTTTTCACATGGATCCTAAATCCGTGAAGGATTT
ATCACAGCATACGGTCTTAGAAATCATTTGCACAGAAGAGTACTCAACAGGGTATACAACAAATACCA
AAATAATCCACTTTGAAATCAAAGACAGGTTCGCCTTTTTTGCTGGACCTCGCCTACGCAATATGGCT
TCCCTCTACGGACAGCTGGATACAACCAAGAAACTCAGAGATTTCTTTACAGTCACAGACCTGAGGAT
AAGGCTGTTAAGACCAGCCGTTGGGGAAATATTTGTAGATGAGCTACACTTGGCACGCTACTTTTACG
CGATCTCAGACATAAAGGTGCGAGGAAGGTGCAAGTGTAATCTCCATGCCACTGTATGTGTGTATGAC
AACAGCAAATTGACATGCGAATGTGAGCACAACACTACAGGTCCAGACTGTGGGAAATGCAAGAAGAA
TTATCAGGGCCGACCTTGGAGTCCAGGCTCCTATCTCCCCATCCCCAAAGGCACTGCAAATACCTGTA
TCCCCAGTATTTCCAGTATTGGTAATCCTCCAAAGTTTAATAGGATATGGCCGAATATTTCTTCCCTT
GAGGTTTCTAACCCAAAACAAGTTGCTCCCAAATTAGCTTTGTCAACAGTTTCTTCTGTTCAAGTTGC
AAACCACAAGAGAGCGAATGTCTGCGACAACGAGCTCCTGCACTGCCAGAACGGAGGGACGTGCCACA
ACAACGTGCGCTGCCTGTGCCCGGCCGCATACACGGGCATCCTCTGCGAGAAGCTGCQGTGCGAGGAG
GCTGGCAGCTGCGGCTCCGACTCTGGCCAGGGCGCGCCCCCGCACGGCTCCCCAGCGCTGCTGCTGCT
GACCACGCTGCTGGGAACCGCCAGCCCCCTGGTGTTC
NOV14e,
CG51051-09
Protein Sequence SEQ ID NO: 170 480 aa MW at 53945.0kD
MYLSRFLSIHALWVTVSSVMQPYPLVWGHYDLCKTQIYTEEGKVWDYMACQPESTDMTKYLKVKLDPP
DITCGDPPETFCAMGNPYMCNNECDASTPELAHPPELMFDFEGRHPSTFWQSATWKEYPKPLQAAITL
SWSKTIELTDNIVITFESGRPDQMILEKSLDYGRTWQPYQYYATDCLDAFHMDPKSVAALSQHTVLEI
ICTEEYSTGYTTNSKIIHFEIKDRFAFFAGPRLRNMASLYGQLDTTKKLRDFFTVTDLRIRLLRPAVG
EIFVDELNLARYFYAISDIKVRGRCKCNLUATVCVYDNSKLTCECEHNTTGPDCGKCKAAYQGRPWSP
GSYLPIPKGTANTCIPSISSTUNPPKFNRIWPNISSLEVSNPKQVAPKLALSTVSSVQVAAHKRANVC
DNELLHCQNCGTCHNNVRCLCPAAYTGILCEKLRCEEAGSCGSDSGQCAPPHGSPALLLLTTLLCTAS
PLVF
NOV14f,
304965116 SEQ ID NO: 171 1465 bp
DNA Sequence ORF Start: at 2 ORF Stop: end of sequence
CACCAGATCTCCCACCATGTATTTGTCAAGATTCCTGTCGATTCATGCCCTTTGGGTTACGGTGTCCT
CAGTGATGCAGCCCTACCCTTTGGTTTGGGGACATTATGATTTGTGTAAGACTCAGATTTACACGGAA
GAACGGAAAGTTTGGGATTACATGGCCTGCCAGCCGGAATCCACGGACATGACAAAATATCTGAAAGT
GAAACTCGATCCTCCCGATATTACCTGTGGAGACCCTCCTGACACGTTCTGTGCAATGGGCAATCCCT
ACATGTGCAATAATCAGTGTGATGCGAGTACCCCTGAGCTGGCACACCCCCCTGAGCTGATGTTTGAT
TTTGAAGGAAGACATCCCTCCACATTTTGGCAGTCTGCCACTTGGAAGGAGTATCCCAAGCCTCTCCA
GGTTAACATCACTCTGTCTTGGAGCAAAACCATTGAGCTAACAGACAACATAGTTATTACCTTTGAAT
CTGGGCGTCCAGACCAAATGATCCTGGAGAAGTCTCTCGATTATGGACGAACATGGCAGCCCTATCAG
TATTATGCCACAGACTGCTTAGATGCTTTTCACATGGATCCTAAATCCGTGAAGGATTTATCACAGCA
TACGGTCTTAGAAATCATTTGCACAGAAGAGTACTCAACAGGGTATACAACAAATAGCAAAATAATCC
ACTTTGAAATCAAAGACAGGTTCGCGTTTTTTGCTGGACCTCGCCTACGCAATATGGCTTCCCTCTAC
GGACAGCTGGATACAACCAAGAAACTCAGAGATTTCTTTACAGTCACAGACCTGAGGATAAGGCTGTT
AAGACCAGCCGTTGGGGAAATATTTGTAGATGAGCTACACTTGGCACGCTACTTTTACGCGATCTCAG
ACATAAAGGTGCGAGGAAGGTGCAAGTGTAATCTCCATGCCACTGTATGTGTGTATCACAACAGCAAA
TTGACATGCGAATGTGAGCACAACACTACAGGTCCAGACTGTGGGAAATGCAAGAAGAATTATCAGGG
CCGACCTTGGAGTCCAGGCTCCTATCTCCCCATCCCCAAAGGCACTGCAAATACCTGTATCCCCAGTA
TTTCCAGTATTGGTAATCCTCCAAAGTTTAATAGGATATGGCCGAATATTTCTTCCCTTGAGGTTTCT
AACCCAAAACAAGTTGCTCCCAAATTAGCTTTGTCAACAGTTTCTTCTGTTCAAGTTGCAAACCACAA
GAGAGCGAATGTCTGCGACAACGAGCTCCTGCACTGCCAGAACGGAGGGACGTGCCACAACAACGTGC
GCTGCCTGTGCCCGGCCGCATACACGGGCATCCTCTGCGAGAAGCTGCGGTGCGAGGAGGCTGGCAGC
TGCGGCTCCGACTCTGGCCAGGGCGCGCCCCCGCACGGCTCCCCAGCGCTGCTGCTGCTGACCACGCT
GCTGGGAACCGCCAGCCCCCTGGTGTTCCTCGAGGGC
NOV14f,
304965116
Protein Sequence SEQ ID NO: 172 488 aa MW at 54786.9 kD
TRSPTMYLSRFLSIHALWVTVSSVMQPYPLVWGHYDLCKTQIYTEEGKAADYAACQPESTDMTKYLKV
KLDPPDITCGDPPETFCAMGNPYMCNNECDASTPELAAPPELMFDFEGRHPSTFWQSATWKEYPKPLQ
VNITLSWSKTIELTDNIVITFESGRPDQMILEKSLDYGRTWQPYQYYATDCLDAFHMDPKSVKDLSQH
TVLEIICTEEYSTGYTTNSKIIHFEIKDRFAFFAGPRLRNAASLYGQLDTTKKLRDFFTVTDLRIRLL
RPAVGEIFVDELHLARYFYAISDIKVRGRCKCNLHATVCVYDNSKLTCECEHNTTGPDCGKCKAAYQG
RPWSPGSYLPIPKGTANTCIPSISSTGNPPKFNRIWPNISSLEVSNPKQVAPKLALSTVSSVQVAAHK
RANVCDNELLHCQNGGTCHNNVRCLCPAAYTGILCEAARCEEAGSCGSDSGQGAPPHGSPALLLLTTL
LGTASPLVFLEG
NOV14g,
273711018 SEQ ID NO: 173 2818 bp
DNA Sequence ORF Start: at 2 ORF Stop: end of sequence
CACCAGATCTAGGCAGCGGTCACGTGGGAGGTCCATCTCTAGGGGCAGACACGCTCGGACCCACCCGC
AGACGGCCCTTCTGGAGAGTTCCTGTGAGAACAAGCGGCCAGACCTGGTTTTCATCATTGACAGCTCT
CGCAGTGTCAACACCCATGACTATGCAAAGGTCAAGGAGTTCATCGTGGACATCTTGCAATTCTTGGA
CATTGGTCCTGATGTCACCCGAGTGGGCCTGCTCCAATATGGCAGCACTGTCAAGAATGAGTTCTCCC
TCAAGACCTTCAAGAGGAAGTCCGAGGTGGAGCGTGCTGTCAAGAGGATGCGGCATCTGTCCACGGGC
ACCATGACTGGGCTGGCCATCCAGTATGCCCTGAACATCGCATTCTCAGAAGCAGAGGGGGCCCGGCC
CCTGAGGGAGAATGTGCCACGGGTCATAATGATCGTGACAGATGGGAGACCTCAGGACTCCGTGGCCG
AGGTGGCTGCTAAGGCACGGGACACGGGCATCCTAATCTTTGCCATTGGTGTGGGCCAGGTAGACTTC
AACACCTTGAAGTCCATTGGGAGTGAGCCCCATGAGGACCATGTCTTCCTTGTGGCCAATTTCAGCCA
GATTGAGACGCTGACCTCCGTGTTCCAGAAGAAGTTGTGCACGGCCCACATGTGCAGCACCCTGGAGC
ATAACTGTGCCCACTTCTGCATCAACATCCCTGGCTCATACGTCTGCAGGTGCAAACAAGGCTACATT
CTCAACTCGGATCAGACGACTTGCAGAATCCACGATCTGTGTGCCATGGAGGACCACAACTGTGAGCA
GCTCTGTGTGAATGTGCCGGGCTCCTTCGTCTGCCAGTGCTACAGTGGCTACGCCCTGGCTGAGGATG
GGAAGAGGTGTGTGGCTGTGGACTACTGTGCCTCAGAAAACCACGGATGTGAACATGAGTGTGTAAAT
GCTGATGGCTCCTACCTTTGCCAGTGCCATGAAGGATTTGCTCTTAACCCAGATGAAAAAACGTGCAC
AAAGATAGACTACTGTGCCTCATCTAATCACGGATGTCAGCACGAGTGTGTTAACACAGATGATTCCT
ATTCCTCCCACTGCCTGAAAGGCTTTACCCTGAATCCAGATAAGAAAACCTGCAGAAGGATCAACTAC
TGTGCACTGAACAAACCCGGCTGTGAGCATGAGTGCGTCAACATGGAGGAGAGCTACTACTGCCGCTG
CCACCGTGGCTACACTCTGGACCCCAATGGCAAAACCTGCAGCCGAGTGGACCACTGTGCACAGCAGG
ACCATGGCTGTGAGCAGCTGTGTCTGAACACGGAGGATTCCTTCGTCTGCCAGTGCTCAGAAGGCTTC
CTCATCAACGAGGACCTCAAGACCTGCTCCCGGGTGGATTACTGCCTGCTGAGTGACCATGGTTGTGA
ATACTCCTCTGTCAACATGGACAGATCCTTTGCCTGTCAGTGTCCTGAGGGACACGTGCTCCGCAGCG
ATGGGAAGACGTGTGCAAAATTGGACTCTTGTGCTCTGGGGGACCACGGTTGTGAACATTCGTGTGTA
AGCAGTGAAGATTCGTTTGTCTGCCAGTGCTTTGAAGGTTATATACTCCGTGAAGATGGAAAAACCTG
CAGAAGGAAAGATGTCTGCCAAGCTATAGACCATGGCTGTGAACACATTTGTGTGAACAGTGACGACT
CATACACGTGCGAGTGCTTGGAGGGATTCCGGCTCGCTGAGGATGGGAAACGCTGCCGAAGGAAGGAT
GTCTGCAAATCAACCCACCATGGCTGCGAACACATTTGTGTTAATAATGGGAATTCCTACATCTGCAA
ATGCTCAGAGGGATTTGTTCTAGCTGAGGACGGAAGACGGTGCAAGAAATGCACTGAAGGCCCAATTG
ACCTGGTCTTTGTGATCGATGGATCCAAGAGTCTTGGAGAAGAGAATTTTGAGGTCGTGAAGCAGTTT
GTCACTGGAATTATAGATTCCTTGACAATTTCCCCCAAAGCCGCTCGAGTGGGGCTGCTCCAGTATTC
CACACAGGTCCACACAGAGTTCACTCTGAGAAACTTCAACTCAGCCAAAGACATGAAAAAAGCCGTGG
CCCACATGAAATACATGGGAAAGGGCTCTATGACTGGGCTGGCCCTGAAACACATGTTTGAGAGAAGT
TTTACCCAAGGAGAAGGGGCCAGGCCCCTTTCCACAAGGGTGCCCAGAGCAGCCATTGTGTTCACCGA
CGGACGGGCTCAGGATGACGTCTCCGAGTGGGCCAGTAGCCAAAAGGCCAATGGTATCACTATGTATG
CTGTTGGGGTAGGAAAAGCCATTGAGGAGGAACTACAAGAGATTGCCTCTGAGCCCACAAACAAGCAT
CTCTTCTATGCCGAAGACTTCAGCACAATGGATGAGATAAGTGAAAAACTCAAGAAAGGCATCTGTGA
AGCTCTAGAAGACTCCGATGGAAGACAGGACTCTCCAGCAGGGGAACTGCCAAAAACGGTCCAACAGC
CAACAGAATCTGAGCCAGTCACCATAAATATCCAAGACCTACTTTCCTGTTCTAATTTTGCAGTGCAA
CACAGATATCTGTTTGAAGAAGACAATCTTTTACGGTCTACACAAAAGCTTTCCCATTCAACAAAACC
TTCAGGAAGCCCTTTGGAAGAAAAACACGATCAATGCAAATGTGAAAACCTTATAATGTTCCAGAACC
TTGCAAACGAAGAAGTAAGAAAATTAACACAGCGCTTAGAAGAAATGACACAGAGAATGGAAGCCCTG
GAAAATCGCCTGAGATACAGAGTCGACGGC
NOV14g,
273711018
Protein Sequence SEQ ID NO: 174 939 aa MW at 104995.2 kD
TRSRQRSRGRSISRGRHARTHPQTALLESSCENKRADLVFIIDSSRSVNTHDYAKVKEFIVDILQFLD
IGPDVTRVGLLQYGSTVKNEFSLKTFKRKSEVERAVKRMRHLSTGTMTGLAIQYALNIAFSEAEGARP
LRENVPRVIMIVTDGRPQDSVAEVAAKAADTGILIFAIGVGQVDFNTLKSIGSEPHEDHVFLVAAFSQ
IETLTSVFQKKLCTAHMCSTLEHNCAHFCINIPGSYVCRCKQGYILNSDQTTCRIQDLCAAEDHNCEQ
LCVNVPGSFVCQCYSGYALAEDGKRCVAVDYCASENHGCEHECAAADGSYLCQCHEGFALNPDEKTCT
KIDYCASSNHGCQHECAATDDSYSCHCLKGFTLNPDKKTCRRINYCAANKPGCEHECAAMEESYYCRC
HRGYTLDPNGKTCSRVDHCAQQDHGCEQLCLNTEDSFVCQCSEGFLINEDLKTCSRVDYCLLSDHGCE
YSCVNMDRSFACQCPEGHVLRSDGKTCAKLDSCALGDHGCEHSCVSSEDSFVCQCFEGYILREDGKTC
RRKDVCQAIDHGCEHICVNSDDSYTCECLEGFRLAEDGKRCRRKDVCKSTHNGCEHICVNNGNSYTCK
CSEGFVLAEDGRRCKKCTEGPIDLVFVIDGSKSLGEENFEAAKQFAAGIIDSLTISPKAARVGLLQYS
TQVHTEFTLRNFNSAKDMKKAVAHMKYMGKGSMTGLALKHMFERSFTQGEGARPLSTRVPRAAIVFTD
GRAQDDVSEWASKAKANGITMYAVGVGKAIEEELQEIASEPTNKHLFYAEDFSTMDEISEKLKKGICE
ALEDSDGRQDSPAGELPKTVQQPTESEPVTINIQDLLSCSNFAVQHRYLFEEDNLLRSTQKLSHSTKP
SGSPLEEAADQCKCENLIMFQNLAAEEVRKLTQRLEEMTQAAEAAENRLRYRVDG
NOV 14h,
273711053 SEQ ID NO: 175 2347 bp
DNA Sequence ORF Start: at 2 ORF Stop: end of sequence
CACCAGATCTAGGCAGCGGTCACGTGGGAGCTCCATCTCTAGGGGCAGACACGCTCGACCCACCCGCG
AGACGGCCCTTCTGGAGAGTTCCTGTGAGAACAAGCGGGCAGACCTGGTTTTCATCATTGACAGCTCT
CGCAGTGTCAACACCCATGACTATGCAAAGGTCAAGGAGTTCATCGTGGACATCTTGCAATTCTTGGA
CATTGGTCCTGATGTCACCCGACTGGGCCTGCTCCAATATGGCAGCACTGTCAAGAATGAGTTCTCCC
TCAAGACCTTCAAGAGGAAGTCCGAGGTGGAGCGTGCTGTCAAGAGGATGCGGCATCTGTCCACGGGC
ACCATGACCGGGCTGGCCATCCAGTATCCCCTGAACATCGCATTCTCAGAAGCAGAGGGGGCCCGGCC
CCTGACGGAGAATGTGCCACGGGTCATAATGATCGTGACAGATGGGAGACCTCAGGACTCCGTGGCCG
AGGTGGCTGCTAAGGCACGGGACACCGGCATCCTAATCTTTGCCATTGGTGTGGGCCAGGTAGACCTC
AACACCTTGAAGTCCATTCGGAGTGAGCCCCATGAGGACCATGTCTTCCTTGTGGCCAATTTCAGCCA
GATTGAGACGCTGACCTCCGTGTTCCAGAAGAAGTTGTGCACGGCCCACATGTGCAGCACCCTGGAGC
ATAACTGTGCCCACTTCTGCATCAACATCCCTGGCTCATACGTCTGCAGGTGCAAACAAGGCTACATT
CTCAACTCGGATCAGACGACTTGCAGAATCCAGGATCTGTGTGCCATGGAGGACCACAACTGTGAGCA
GCTCTGTGTGAATGTGCCCGGCTCCTTCGTCTGCCAGTGCTACAGTGGCTACGCCCTGGCTGAGGATG
GGAAGAGGTGTGTGGCTGTGGACTACTGTGCCTCAGAAAACCACGGATGTGAACATGAGTGTGTAAAT
GCTGATGGCTCCTACCTTTGCCAGTGCCATCAAGGATTTGCTCTTAACCCAGATGAAAAAACGTGCAC
AAAGATAGACTACTGTGCCTCATCTAATCACGGATGTCAGCACGAGTGTGTTAACACAGATGATTCCT
ATTCCTGCCACTGCCTGAAAGGCTTTACCCTGAATCCAGATAAGAAAACCTGCAGAAGGATCAACTAC
TGTGCACTGAACAAACCGGGCTGTGAGCATGAGTGCGTCAACATGGAGGAGAGCTACTACTGCCGCTG
CCACCGTGGCTACACTCTGGACCCCAATGGCAAAACCTGCAGCCGAGTGGACCACTGTGCACAGCAGG
ACCATGGCTGTGAGCAGCTGTGTCTGAACACGGAGGATTCCTTCGTCTGCCAGTGCTCAGAAGGCTTC
CTCATCAACGAGGACCTCAAGACCTCCTCCCGGGTGGATTACTGCCTGCTGAGTGACCATGGTTGTGA
ATACTCCTGTGTCAACATGGACAGATCCTTTGCCTGTCAGTGTCCTGAGGGACACGTGCTCCGCAGCG
ATGGGAAGACGTGTGCAAAATTGGACTCTTGTGCTCTGGGGGACCACGGTTGTGAACCAGTGACGACT
AGCAGTGAAGATTCGTTTGTGTGCCAGTGCTTTGAAGGTTATATACTCCGTGAAGATGGAAAAACCTG
CAGAAGGAAAGATGTCTGCCAAGCTATAGACCATGGCTGTGAACACATTTGTGTGAACAGTGACGACT
CATACACGTGCGAGTGCTTGGAGGGATTCCGGCTCGCTGAGGATGGGAAACGCTGCCGAAGGAAGGAT
GTCTGCAAATCAACCCACCATGGCTGCGAACACATTTGTGTTAATAATGGGAATTCCTACATCTGCAA
ATGCTCAGAGGGATTTGTTCTAGCTGAGGACGGAAGACGGTGCAAGAGTATCACTATGTATGCTGTTG
GGGTAGGAAAAGCCATTGAGGAGGAACTACAAGAGATTGCCTCTGAGCCCACAAACAAGCATCTCTTC
TATGCCGAAGACTTCAGCACAATGGATGAGATAAGTGAAAAACTCAAGAAAGGCATCTGTGAAGCTCT
AGAAGACTCCGATGGAAGACAGGACTCTCCAGCAGGGGAACTGCCAAAAACGGTCCAACAGCCAACAG
TGCAACACAGATATCTGTTTGAAGAAGACAATCTTTTACGGTCTACACAAAAGCTTTCCCATTCAACA
AAACCTTCAGGAAGCCCTTTGGAAGAAAAACACGATCAATGCAAATGTGAAAACCTTATAATGTTCCA
GAACCTTGCAAACGAAGAAGTAAGAAAATTAACACAGCGCTTAGAAGAAATGACACAGAGAATGGAAG
CCCTGGAAAATCGCCTGAGATACAGAGTCGACGGC
NOV14h,
273711053
Protein Sequence SEQ ID NO: 176 782 aa MW at 87838.8 kD
TRSRQRSRGRSISRGRHARTHPQTALLESSCENKRADLVFIIDSSRSVNTHDYAKVKEFIVDILQFLD
IGPDVTRVGLLQYGSTVKNEFSLKTFKRKSEVERAVKRMRHLSTGTMTGLAIQYALNIAFSEAEGARP
LRENVPRVIMIVTDGRPQDSVAEVAAKARDTGILIFAIGVGQVDFNTLKSIGSEPHEDHVFLVANFSQ
IETLTSVFQKKLCTAHMCSTLEHNCAHFCINIPGSYVCRCKQGYILNSDQTTCRIQDLCAMEDHNCEQ
LCVNVPGSFVCQCYSGYALAEDGKRCVAVDYCASENHGCEHECVNADGSYLCQCHEGFALNPDEKTCT
KIDYCASSNHGCQHECVNTDDSYSCHCLKGFTLNPDKKTCRRINYCALNKPGCEHECVNMEESYYCRC
HRGYTLDPNGKTCSRVDHCAQQDHGCEQLCLNTEDSFVCQCSEGFLINEDLKTCSRVDYCLLSDHGCE
YSCVNMDRSFACQCPEGHVLRSDGKTCAKLDSCALGDHGCEHSCVSSEDSFVCQCFEGYILREDGKTC
RRKDVCQAIDHGCEHICVNSDDSYTCECLEGFRLAEDGKRCRRKDVCKSTHHGCEHICVNNGNSYICK
CSEGFVLAEDGRRCKSITMYAVGVGKAIEEELQEIASEPTNKHLFYAEDFSTMDEISEKLKKGICEAL
EDSDGRQDSPAGELPKTVQQPTVQHRYLFEEDNLLRSTQKLSHSTKPSGSPLEEKHDQCKCENLIMFQ
NLANEEVRKLTQRLEEMTQRMEALENRLRYRVDG
NOV14i,
274051275 SEQ ID NO: 177 2761 bp
DNA Sequence ORF Start: at 2 ORF Stop: end of sequence
CACCAGATCTAGGCAGCGGTCACGTGGGAGGTCCATCTCTAGCGGCAGACACGCTCGGACCCACCCGC
AGACGGCCCTTCTGGAGAGTTCCTGTGAGAACAAGCGGGCAGACCTGGTTTTCATCATTGACAGCTCT
CGCAGTGTCAACACCCATOACTATGCAAAGGTCAACGAGTTCATCGTGGACATCTTGCAATTCTTGGA
CATTGGTCCTGATGTCACCCGAGTGGGCCTGCTCCAATATGGCAGCACTGTCAAGAATGAGTTCTCCC
TCAAGACCTTCAAGAGGAAGTCCGAGGTGGAGCGTGCTGTCAAGAGGATGCGGCATCTGTCCACGGGC
ACCATGACCGGGCTGGCCATCCAGTATGCCCTGAACATCGCATTCTCAGAAGCAGAGGGGGCCCGGCC
CCTGAGGGAGAATGTGCCACGGGTCATAATCATCGTGACAGATGGGAGACCTCAGCACTCCGTGGCCG
AGGTGGCTGCTAAGGCACGGGACACGGGCATCCTAATCTTTGCCATTGGTGTGGGCCAGGTAGACTTC
AACACCTTGAAGTCCATTGGGAGTGAGCCCCATGAGGACCATGTCTTCCTTGTGGCCAATTTCAGCCA
GATTGAGACGCTGACCTCCGTGTTCCAGAAGAAGTTGTGCACGGCCCACATGTGCAGCACCCTGGAGC
ATAACTGTGCCCACTTCTGCATCAACATCCCTCGCTCATACGTCTGCAGGTCCAAACAAGGCTACATT
CTCAACTCGGATCAGACGACTTGCACAATCCAGGATCTGTGTGCCATGGAGGACCACAACTGTGAGCA
GCTCTGTGTGAATGTGCCGGGCTCCTTCGTCTGCCAGTGCTACAGTGGCTACGCCCTGGCTGAGGATG
GGAAGAGGTGTGTGGCTGTGGACTACTGTGCCTCAGAAAACCACGGATGTGAACATGAGTGTGTAAAT
GCTGATGGCTCCTACCTTTGCCAGTGCCATGAAGGATTTGCTCTTAACCCAGATGAAAAAACGTGCAC
AAAGATAGACTACTGTGCCTCATCTAATCACGGATGTCAGCACGAGTGTGTTAACACAGATGATTCCT
ATTCCTGCCACTGCCTGAAAGGCTTTACCCTGAATCCAGATAAGAAAACCTGCAGAAGGATCAACTAC
TGTGCACTGAACAAACCGGGCTGTGAGCATGAGTCCGTCAACATGGAGGAGAGCTACTACTGCCGCTG
CCACCGTGGCTACACTCTGGACCCCAATGGCAAAACCTGCAGCCGAGTGGACCACTGTGCACAGCAGG
ACCATGGCTGTGAGCAGCTGTGTCTGAACACGGAGGATTCCTTCGTCTGCCAGTGCTCAGAAGGCTTC
CTCATCAACGAGGACCTCAAGACCTGCTCCCGGGTGGATTACTGCCTGCTCAGTGACCATGGTTGTGA
ATACTCCTGTGTCAACATGGACAGATCCTTTGCCTGTCAGTGTCCTGAGGGACACGTGCTCCGCAGCG
ATGGGAAGACGTGTGCAAAATTGGACTCTTGTGCTCTGGGGGACCACGGTTGTGAACATTCGTGTGTA
AGCAGTGAAGATTCGTTTGTGTGCCAGTGCTTTGAAGGTTATATACTCCGTGAAGATGGAAAAACCTG
CAGAAGGAAAGATGTCTGCCAAGCTATAGACCATGGCTGTGAACACATTTGTGTGAACAGTGACGACT
CATACACGTGCGAGTGCTTGGAGGGATTCCGGCTCGCTGAGGATGGGAAACGCTGCCGAAGGAAGGAT
GTCTGCAAATCAACCCACCATGGCTGCGAACACATTTGTGTTAATAATGGGAATTCCTACATCTGCAA
ATGCTCAGAGCGATTTGTTCTAGCTGAGGACGGAAGACGGTGCAAGAAATGCACTGAAGGCCCAATTG
ACCTGGTCTTTGTGATCGATGGATCCAAGAGTCTTGGAGAAGAGAATTTTGAGGTCGTGAAGCAGTTT
GTCACTGGAATTATAGATTCCTTGACAATTTCCCCCAAAGCCGCTCGAGTGGGGCTGCTCCAGTATTC
CACACAGGTCCACACAGAGTTCACTCTGAGAAACTTCAACTCAGCCAAAGACATGAAAAAAGCCGTGG
CCCACATGAAATACATGGGAAAGGGCTCTATGACTGGGCTGGCCCTGAAACACATGTTTGAGAGAAGT
TTTACCCAAGGAGAAGGGGCCAGGCCCCTTTCCACAAGGGTGCCCAGAGCAGCCATTGTGTTCACCGA
CGCACGGGCTCAGGATGACGTCTCCGAGTGGGCCAGTAAAGCCAAGGCCAATGGTATCACTATGTATG
CTGTTGGGGTAGGAAAAGCCATTGAGGAGGAACTACAAGAGATTGCCTCTGAGCCCACAAACAAGCAT
CTCTTCTATGCCGAAGACTTCAGCACAATGGATGAGATAAGTGAAAAACTCAAGAAAGGCATCTGTGA
AGCTCTAGAAGACTCCGATGGAAGACAGGACTCTCCAGCAGGGGAACTGCCAAAAACGGTCCAACAGC
CAACAGTGCAACACAGATATCTGTTTGAAGAAGACAATCTTTTACGGTCTACACAAAACCTTTCCCAT
TCAACAAAACCTTCAGGAAGCCCTTTGGAAGAAAAACACGATCAATGCAAATGTGAAAACCTTATAAT
GTTCCAGAACCTTGCAAACGAAGAAGTAAGAAAATTAACACAGCGCTTAGAAGAAATGACACAGAGAA
TGGAAGCCCTGGAAAATCGCCTGAGATACAGAGTCGACGGC
NOV14i,
274051275
Protein Sequence SEQ ID NO: 178 1920 aa MW at 102933.0 kD
TRSRQRSRGRSISRGRHARTHPQTALLESSCENKRADLVFIIDSSRSVNTHDYAKVKEFIVDILQFLD
IGPDVTRVGLLQYGSTVKNEFSLKTFKRKSEVERAVKRMRHLSTGTMTGLAIQYALNIAFSEAEGARP
LRENVPRVIMIVTDGRPQDSVAEVAAKARDTGILIFAIGVGQVDFNTLKSIGSEPHEDHVFLVANFSQ
IETLTSVFQKKLCTAHMCSTLEHNCAHFCINIPGSYVCRCKQGYILNSDQTTCRIQDLCAMEDHNCEQ
LCVNVPGSFVCQCYSGYALAEDGKRCVAVDYCASENHGCEHECVNADGSYLCQCHEGFALNPDEKTCT
KIDYCASSNHGCQHECVNTDDSYSCHCLKGFTLNPDKKTCRRINYCALNKPGCEHECVNMEESYYCRC
HRGYTLDPNGKTCSRVDHCAQQDHGCEQLCLNTEDSFVCQCSEGFLINEDLKTCSRVDYCLLSDHGCE
YSCVNMDRSFACQCPEGHVLRSDCKTCAKLDSCALGDHGCEHSCVSSEDSFVCQCFEGYILREDGKTC
RRKDVCQAIDHGCEHICVNSDDSYTCECLEGFRLAEDGKRCRRKDVCKSTHHGCEHICVNNGNSYICK
CSEGFVLAEDGRRCKKCTEGPIDLVFVIDGSKSLGEENFEVVKQFVTGIIDSLTISPKAARVGLLQYS
TQVHTEFTLRNFNSAKDMKKAVAHMKYMGKGSMTGLALKHMFERSFTQGEGARPLSTRVPRAAIVFTD
GRAQDDVSEWASKAKANGITMYAVGVGKAIEEELQEIASEPTNKHLFYAEDFSTMDEISEKLKKGICE
ALEDSDGRQDSPACELPKTVQQPTVQHRYLFEEDNLLRSTQKLSHSTKPSGSPLEEKHDQCKCENLIM
FQNLANEEVRKLTQRLEEMTQRMEALENRLRYRVDG
NOV14j,
CG51051-01 SEQ ID NO: 179 1908 bp
DNA Sequence ORF Start: ATG at 230 ORF Stop: TAG at 1670
GGCTTCCACCAAAGTCCTCAATATACCTGAATACGCACAATATCTTAACTCTTCATATTTGGTTTTGG
GATCTGCTTTGAGGTCCCATCTTCATTTAAAAAAAAATACAGAGACCTACCTACCCGTACGCATACAT
ACATATGTGTATATATATGTAAACTAGACAAAGATCGCAGATCATAAAGCAAGCTCTGCTTTAGTTTC
CAAGAAGATTACAAAGAATTTAGACATGTATTTGTCAAGATTCCTGTCGATTCATGCCCTTTGGGTTA
CGGTGTCCTCAGTCATGCACCCCTACCCTTTGGTTTGGGCACATTATGATTTGTGTAAGACTCAGATT
TACACGGAAGAAGGGAAAGTTTGGGATTACATGGCCTGCCAGCCGGAATCCACGGACATGACAAAATA
TCTGAAAGTGAAACTCGATCCTCCGGATATTACCTGTGGAGACCCTCCTGAGACGTTCTGTGCAATGG
GCAATCCCTACATGTGCAATAATGAGTGTGATGCGAGTACCCCTGAGCTGGCACACCCCCCTGAGCTG
ATGTTTGATTTTGAAGGAAGACATCCCTCCACATTTTGGCAGTCTGCCACTTGGAAGGAGTATCCCAA
GCCTCTCCAGGTTAACATCACTCTGTCTTGGAGCAAAACCATTGAGCTAACAGACAACATAGTTATTA
CCTTTGAATCTGGGCGTCCAGACCAAATGATCCTGGAGAAGTCTCTCCATTATGGACGAACATGGCAG
CCCTATCAGTATTATGCCACAGACTGCTTAGATGCTTTTCACATCGATCCTAAATCCGTGAAGGATTT
ATCACAGCATACGGTCTTAGAAATCATTTGCACAGAAGAGTACTCAACAGGGTATACAACAAATAGCA
AAATAATCCACTTTGAAATCAAAGACAGGTTCGCGTTTTTTGCTGGACCTCGCCTACGCAATATGGCT
TCCCTCTACGGACAGCTGGATACAACCAAGAAACTCAGAGATTTCTTTACAGTCACAGACCTGAGGAT
AAGGCTGTTAAGACCAGCCGTTGGGGAAATATTTGTAGATGAGCTACACTTGGCACGCTACTTTTACG
CGATCTCAGACATAAAGGTGCGAGGAAGGTGCAAGTGTAATCTCCATGCCACTCTATGTGTGTATGAC
AACAGCAAATTGACATGCGAATGTGAGCACAACACTACAGGTCCAGACTGTGGGAAATGCAAGAAGAA
TTATCAGGGCCGACCTTGGAGTCCAGGCTCCTATCTCCCCATCCCCAAAGGCACTGCAAATACCTGTA
TCCCCAGTATTTCCAGTATTGGTAATCCTCCAAAGTTTAATAGGATATGGCCGAATATTTCTTCCCTT
GAGGTTTCTAACCCAAAACAAGTTGCTCCCAAATTAGCTTTGTCAACAGTTTCTTCTGTTCAAGTTGC
AAACCACAAGAGAGCGAATGTCTGCGACAACGAGCTCCTGCACTGCCAGAACGGAGGGACGTGCCACA
ACAACGTGCGCTGCCTGTGCCCGGCCGCATACACGGGCATCCTCTGCGAGAAGCTGCGGTGCGAGGAG
GCTGGCAGCTGCGGCTCCGACTCTGGCCAGGGCGCGCCCCCGCACGGCTCCCCAGCGCTGCTGCTGCT
GACCACGCTGCTGGGAACCGCCAGCCCCCTGGTGTTCTAGGTGTCACCTCCAGCCACACCGGACGGGC
CTGTGCCGTGGGGAAGCAGACACAACCCAAACATTTGCTACTAACATAGGAAACACACACATACAGAC
ACCCCCACTCAGACAGTGTACAAACTAAGAAGGCCTAACTGAACTAAGCCATATTTATCACCCGTGGA
CAGCACATCCGAGTCAGGACTGTTAATTTCTGACTCCAGAGGAGTTGCCAGCTGTTGATATTATCACT
GCAA
NOV14j,
CG51051-01
Protein Sequence SEQ ID NO: 180 480 aa MW at 53945.0 kD
MYLSRFLSIHALWVTVSSVMQPYPLVWGHYDLCKTQIYTEEGKVWDYMACQPESTDMTKYLKVKLDPP
DITCGDPPETFCANGNPYMCNNECDASTPELAHPPELMFDFEGRHPSTFWQSATWKEYPKPLQVNITL
SWSKTIELTDNIVITFESGRPDQMILEKSLDYGRTWQPYQYYATDCLDAFHMDPKSVKDLSQHTVLEI
ICTEEYSTGYTTNSKIIHFEIKDRFAFFAGPRLRNMASLYGQLDTTKKLRDFFTVTDLRIRLLRPAVG
EIFVDELHLARYFYAISDIKVRGRCKCNLHATVCVYDNSKLTCECEHNTTGPDCGKCKKNYQGRPWSP
GSYLPIPKGTANTCIPSISSIGNPPKFNRIWPNISSLEVSNPKQVAPKLALSTVSSVQVANHKRANVC
DNELLHCQNGGTCHNNVRCLCPAAYTGILCEKLRCEEAGSCGSDSGQGAPPHGSPALLLLTTLLGTAS
PLVF
NOV14k,
CG51051-02 SEQ ID NO: 181 1343 bp
DNA Sequence ORF Start: ATG at 18 ORF Stop: End of Sequence
ATGTATTTGTCAAGATTCCTGTCGATTCATGCCCTTTGGGTTACGGTGTCC
TCAGTGATGCAGCCCTACCCTTTGGTTTGGGGACATTATGATTTGTGTAAGACTCAGATTTACACGGA
AGAAGGGAAAGTTTGGGATTACATGGCCTGCCAGCCGGAATCCACGGACATGACAAAATATCTGAAAG
TGAAACTCGATCCTCCGGATATTACCTGTGGAGACCCTCCTGAGACGTTCTGTGCAATGGGCAATCCC
TACATGTGCAATAATGAGTGTGATGCGAGTACCCCTGAGCTGGCACACCCCCCTGAGCTGATGTTTGA
TTTTGAAGGAAGACATCCCTCCACATTTTGGCAGTCTGCCACTTGGAAGGAGTATCCCAAGCCTCTCC
AGGTTAACATCACTCTGTCTTGGAGCAAAACCATTGAGCTAACAGACAACATAGTTATTACCTTTGAA
TCTGGGCGTCCAGACCAAATGATCCTGGAGAAGTCTCTCGATTATGGACGAACATGGCAGCCCTATCA
GTATTATGCCACAGACTGCTTAGATGCTTTTCACATGGATCCTAAATCCGTGAAGGATTTATCACAGC
ATACGGTCTTAGAAATCATTTGCACAGAAGAGTACTCAACAGGGTATACAACAAATAGCAAAATAATC
CACTTTGAAATCAAAGACAGGTTCGCGTTTTTTGCTGGACCTCGCCTACGCAATATGGCTTCCCTCTA
CGGACAGCTGGATACAACCAAGAAACTCAGGGATTTCTTTACAGTCACAGACCTGAGGATAAGGCTGT
TAAGACCAGCCGTTGGGGAAATATTTGTAGATGAGCTACACTTGCCACGCTACTTTTACGCGATCTCA
GACATAAAGGTGCGAGGAACGTGCAAGTGTAATCTCCATGCCACTGTATGTGTGTATGACAACAGCAA
ATTGACATGCGAATGTGAGCACAACACTACAGGTCCAGACTGTGGGAAATGCAAGAAGAATTACCAGG
GCCGACCTTCGAGTCCAGGCTCCTATCTCCCCATCCCCAAAGGCGCTGCAAATACCTGTATCCCCAGT
ATTTCCAGTATTGGTAAGTGTTATTGTAACCCTTTGGGCTCAATCCATGATCGTTGTAATGGCTCAGG
ACTACGGCTGTCAACCGAATGTCTGCGACAACGAGCTCCTGCCACCATGTATTTGTCAAGATTCCTGT
CGATTCTATCTCTGGCCAGGGCGCGCCCCCGCACGGCTCCCTCGAGAAGGG
NOV14k,
CG51051-02

| | |
|---|---|
| Protein Sequence | SEQ ID NO: 182 442 aa MW at 50335.9 kD |

MYLSRFLSTHALWVTVSSVMQPYPLVWGHYDLCKTQIYTEEGKVWDYMACQPESTDMTKYLKVKLDPP
DITCGDPPETFCAMGNPYMCNNECDASTPELAAPPELMFDFEGRHPSTFWQSATWKEYPKPLQVNITL
SWSKTIELTDNIVITFESGRPDQMILEKSLDYGRTWQPYQYYATDCLDAFHMDPKSVKDLSQHTVLEI
ICTEEYSTGYTTNSKIIHFEIKDRFAFFAGPRLRNMASLYGQLDTTKAARDFFTAADLRIRLLRPAVG
EIFVDELHLARYPYAISDIAARGRCKCNLHATVCAADNSAATCECENNTTGPDCGKCKAAYQGRPWSP
GSYLPIPKGAANTCIPSISSIGKCYCNPLGSIHDRCNGSGFCECKTGTTGPKCDECLPGNSWHYGCQP
NVCDNELLPPCICQDSCRFYLWPGRAPARLPREG
NOV14l,
CG51051-03 SEQ ID NO: 183 1544 bp
DNA Sequence ORF Start: ATG at 230 ORF Stop: TAG at 1517
GGCTTCCACCAAAGTCCTCAATATACCTGAATACGCACAATATCTTAACTCTTCATATTTGGTTTTGG
GATCTGCTTTGAGGTCCCATCTTCATTTAAAAAAAAATACAGAGACCTACCTACCCGTACGCATACAT
ACATATGTGTATATATATGTAAACTAGACAAAGATCGCAGATCATAAAGCAAGCTCTGCTTTAGTTTC
CAAGAAGATTACAAAGAATTTAGAGATGTATTTGTCAAGATTCCTGTCGATTCATGCCCTTTGGGTTA
CGGTGTCCTCAGTGATGCAGCCCTACCCTTTGGTTTGGGGACATTATGATTTGTGTAAGACTCAGATT
TACACCGAAGAACGGAAAGTTTGGGATTACATGGCCTGCCAGCCGGAATCCACGGACATGACAAAATA
TCTGAAAGTGAAACTCGATCCTCCGCATATTACCTGTGCAGACCCTCCTGAGACGTTCTGTGCAATGG
GCAATCCCTACATGTGCAATAATGAGTGTGATGCGAGTACCCCTGAGCTGGCACACCCCCCTGAGCTG
ATGTTTGATTTTGAAGGAAGACATCCCTCCACATTTTGGCAGTCTGCCACTTGGAAGGAGTATCCCAA
GCCTCTCCAGGTTAACATCACTCTGTCTTGGAGCAAAACCATTGAGCTAACAGACAACATAGTTATTA
CCTTTGAATCTGGGCGTCCAGACCAAATGATCCTGGAGAAGTCTCTCGATTATGGACGAACATGGCAG
CCCTATCAGTATTATGCCACAGACTGCTTAGATGCTTTTCACATGGATCCTAAATCCGTGAAGGATTT
ATCACAGCATACGGTCTTAGAAATCATTTGCACAGAAGAGTACTCAACAGGGTATACAACAAATAGCA
AAATAATCCACTTTGAAATCAAAGACAGGTTCGCGTTTTTTGCTGGACCTCGCCTACGCAATATGGCT
TCCCTCTACGGACAGCTGGATACAACCAAGAAACTCAGAGATTTCTTTACAGTCACAGACCTGAGGAT
AAGGCTGTTAAGACCAGCCGTTGGCGAAATATTTGTAGATGAGCTACACTTGCCACGCTACTTTTACG
CGATCTCAGACATAAAGGCGCGAGGAAGGTGCAAGTGTAATCTCCATGCCACTGTATGTGTGTATGAC
AACAGCAAATTGACATGCGAATGTGAGCACAACACTACAGGTCCAGACTGTGGGAAATGCAAGAAGAA
TTATCAGGGCCGACCTTGGAGTCCAGGCTCCTATCTCCCCATCCCCAAAGGCACTGCAAATACCTCGA
ATGTCTGCGACAACGAGCTCCTGCACTGCCAGAACGCAGGGACCTGCCACAACAACGTGCGCTGCCTG
TGCCCGGCCGCATACACGGGCATCCTCTCCGAGAAGCTGCGGTCCGAGGAGGCTGGCAGCTGCGGCTC
CGACTCTGGTCAGGGCGCGCCCCCGCACGGCTCCCCAGCGCTGCTGCTGCTGACCACGCTGCTGGGAA
CCGCCAGCCCCCTGGTGTTTTAGGTGTCACCTCCAGCCACACCGGACG
MYLSRFLSIHALWVTVSSVMQPYPLVWGHYDLCKTQIYTEEGKVWDYMACQPESTDMTKYLKVKLDPP
DITCGDPPETFCAMGNPYMCNNECDASTPELAAPPELMFDFEGRHPSTFWQSATWKEYPKPLQVNITL
SWSKTIELTDNIVITFESGRPDQMILEKSLDYGRTWQPYQYYATDCLDAFHMDPKSVKDLSQHTVLEI
ICTEEYSTGYTTNSKIIHFEIKDRFAFFAGPRLRNMASLYGQLDTTKKLRDFFTVTDLRIRLLRPAVG
EIFVDELHLARYFYAISDIKARGRCKCNLBATVCVYDNSKLTCECEHNTTGPDCGKCKAAYQGRPWSP
GSYLPIPKGTAATSNVCDNELLHCQNGGTCHNAARCLCPAAYTGILCEKLRCEEAGSCGSDSGQGAPP
HGSPALLLLTTLLGTASPLVF
NOV14m,
CG51051-04 SEQ ID NO: 185 1771 bp
DNA Sequence ORF Start: ATG at 230 ORF Stop: TAG at 1544
GGCTTCCACCAAAGTCCTCAATATACCTGAATACGCACAATATCTTAACTCTTCATATTTGGTTTTGG
GATCTGCTTTGAGGTCCCATCTTCATTTAAAAAAAAATACAGAGACCTACCTACCCGTACGCATACAT
ACATATGTGTATATATATGTAAACTAGACAAAGATCGCAGATCATAAAGCAAGCTCTGCTTTAGTTTC
CAAGAAGATTACAAAGAATTTAGAGATGTATTTGTCAAGATTCCTGTCGATTCATGCCCTTTGGGTTA
CGGTGTCCTCAGTGATGCAGCCCTACCCTTTGGTTTGGGGACATTATGATTTGTGTAAGACTCAGATT
TACACGGAAGAAGGGAAAGTTTGGGATTACATGGCCTGCCAGCCGGAATCCACGGACATGACAAAATA
TCTGAAAGTGAAACTCGATCCTCCGGATATTACCTGTGGAGACCCTCCTGAGACGTTCTGTGCAATGG
GCAATCCCTACATGTGCAATAATGAGTGTGATGCGAGTACCCCTGAGCTGGCACACCCCCCTGAGCTG
ATGTTTGATTTTGAAGGAAGACATCCCTCCACATTTTCGCAGTCTGCCACTTGGAAGGAGTATCCCAA
GCCTCTCCAGGTTAACATCACTCTGTCTTGGAGCAAAACCATTGAGCTAACAGACAACATAGTTATTA
CCTTTGAATCTCGGCGTCCAGACCAAATGATCCTGGAAAAGTCTCTCGATTATGGACGAACATGGCAG
CCCTATCAGTATTATGCCACAGACTGCTTAGATGCTTTTCACATGGATCCTAAATCCGTGAAGGATTT
ATCACAGCATACGGTCTTAGAAATCATTTGCACAGAAGAGTACTCAACAGGGTATACAACAAATAGCA
AAATAATCCACTTTGAAATCAAACACAGGTTCGCGTTTTTTGCTGGACCTCGCCTACGCAATATGGCT
TCCCTCTACGGACAGCTGGATACAACCAAGAAACTCAGAGATTTCTTTACAGTCACAGACCTGAGGCT
AAGGCTGTTAAGACCAGCCGTTGGGGAAATATTTGTAGATGAGCTACACTTGGCACGCTACTTTTACG
CGATCTCAGACATAAAGGTGCGAGGAAGGTGCAAGTGTAATCTCCATGCCACTGTATGTGTGTATGAC
AACAGCAAATTGACATGCGAATGTGAGCACAACACTACAGGTCCAGACTGTGGGAAATGCAAGAAGAA
TTATCAGGGCCGACCTTGGAGTCCAGGCTCCTATCTCCCCATCCCCAAAAGGCACTGCAATACCTGTA
TCCCCACTATTTCCAGTATTGGTACGAATGTCTGCGACAACGAGCTCCTGCACTGCCACAACGGAGGG
ACGTGCCACAACAACGTGCGCTGCCTGTGCCCGGCCGCATACACGGGCATCCTCTGCGAGAAGCTGCG
GTGCGAGGAGGCTGGCAGCTGCGGCTCCGACTCTGGCCAGGCCGCGCCCCCGCACGGCTCCCCAGCGC
TGCTGCTGCTGACCACGCTGCTGGGAACCGCCAGCCCCCTGGTGTTCTAGGTGTCACCTCCAGCCACA
CCGGACGGGCCTGTGCCGTGGGGAAGCAGACACAACCCAAACATTTGCTACTAACATAGGAAACACAC
ACATACAGACACCCCCACTCAGACAGTGTACAAACTAAGAAGGCCTAACTGAACTAAGCCATATTTAT
CACCCGTGGACAGCACATCCGAGTCAAGACTGTTAATTTCTGACTCCAGAGGAGTTGGCAGCTGTTGA
TAT
NOV14n,
CG51051-05
Protein Sequence SEQ ID NO: 188 438 aa MW at 49339.7 kD
MYLSRFLSIHALWVTVSSVMQPYPLVWGHYDLCKTQIYTEEGKVWDYMACQPESTDMTKYLKVKLDPP
DITCGDPPETFCANGNPYMCNNECDASTPELAAPPELMFDFEGRHPSTFWQSATWKEYPKPLQVNITL
SWSKTIELTDNIVITFESGRPDQMILEKSLDYGRTWQPYQYYATDCLDAFHMDPKSVKDLSQHTVLEI
ICTEEYSTGYTTNSKIIHFEIKDRFAFFAGPRLRNMASLYGQLDTTKKLRDFFTAADLRIRLLRPAVG
EIFVDELHLARYFYAISDIKVRGRCKCNLHATVCVYDNSKLTCECEHNTTGPDCGKCKAAYQGRPWSP
GSYLPIPKGTANTCIPSISSIGTNVCDNELLHCQNGGTCHNAARCLCPAAYTGILCEKLRCEEAGSCG
SDSGQGAPPHGSPALLLLTTLLGTASPLVF
NOV14o,
CG51051-06 SEQ ID NO: 189 1290 bp
DNA Sequence ORF Start: ATG at 1 ORF Stop: end of sequence
ATGTATTTGTCAAGATTCCTGTCGATTCATGCCCTTTGGGCTACGGTGTCCTCAGTGATGCAGCCCTA
CCCTTTGGTTTGGGGACATTATGATTTGTGTAAGACTCAGATTTACACGGAAGAAGGGAAACTTTGGG
ATTACATGGCCTGCCAGCCGGAATCCACGCACATGACAAAAAATCTGAAAGTGAAACTCGATCCTCCG
GATATTACCTGTGGAGACCCTCCWGAGACGTTCTGTGCAATGGGCAATCCCTACATGTGCAATAATGA
GTGTGATGCGAGTACCCCTGAGCTGGCACACCCCCCTGAGCTGATGTTTGATTTTGAAGGAAGACATC
CCTCCACATTTTGGCAGTCTGCCACTTGGAAGGAGTATCCCAAGCCTCTCCAGGTTAACATCACTCTG
TCTTGGAGCAAAACCATTGAGCTAACAGACAACATAGTTATTACCTTTGAATCTGGGCGTCCAGACCA
AATCATCCTCGAGAAGTCTCTCGATTATGGACGAACATGGCAGCCCTATCAGTATTATGCCACAGACT
GCTTAGATGCTTTTCACATGGATCCTAAATCCGTGAAGGATTTATCACAGCATACGGTCTTAGAAATC
ATTTGCACAGAAGACTACTCAACAGGGTATACAACAAATAGCAAAATAATCCACTTTGAAATCAAAGA
CAGGTTCGCGTTTTTTGCTGGACCTCGCCTACGCAATATGGCTTCCCTCTACGGACAGCTGGATACAA
CCAAGAAACTCAGAGATTTCTTTACAGTCACAGACCTGAGGATAAGGCTGTTAAGACCAGCCGTTGGG
GAAATATTTGTAGATGAGCTACACTTGGCACGCTACTTTTACGCGATCTCAGACATAAAGGTGCGAGG
AAGGTGCAAGTGTAATCTCCATGCCACTGTATGTGTGTATGACAACAGCAAATTGACATGCGAATGTG
AGCACAACACTACAGGTCCAGACTGTGGGAAATGCAAGAAGAATTATCAGGGCCGACCTTGGAGTCCA
GGCTCCTATCTCCCCATCCCCAAAGGCACTGCAAATACCTGTATCCCCAGTATTTCCAGTATTGGTAC
GAATGTCTGCGACAACGAGCTCCTGCACTGCCAGAACGGAGGGACGTGCCACAACAACGTOCGCTGCC
TGTCCCCGGCCGCATACACGGGCATCCTCTGCGAGAAGCTGCGGTGCGAGGAGGCTGGCAGCTCCGGC
TCCGACTCTCAAGCCAGGGCGCGCCCCCCCACGGCTCCCTCGAGAAGGGCAATTCCACCACACTGGAC
NOV14o,
CG51051-06
Protein Sequence SEQ ID NO: 190 430 aa MW at 48548.6 kD
MYLSRFLSIHALWATVSSVMQPYPLVWGHYDLCKTQIYTEEGKVWDYMACQPESTDMTKYLKVKLDPP
DITCGDPPETFCAMGNPYMCNNECDASTPELAHPPELNFDFEGRHPSTFWQSATWKEYPKPLQAAITL
SWSKTIELTDNIVITFESGRPDQMILEKSLDYGRTWQPYQYYATDCLDAFHMDPKSVKDLSQHTVLEI
ICTEEYSTGYTTNSKIIHFEIKDRFAFFAGPRLRNMASLYGQLDTTKKLRDFFTVTDLRIRLLRPAVG
EIFVDELHLARYFYAISDIKVRGRCKCNLHATVCVYDNSKLTCECEHNTTGPDCGKCKKNYQGRPWSP
GSYLPIPKGTANTCIPSISSIGTNVCDNELLHCQNGGTCHNNVRCLCPAAYTGILCEKLRCEEAGSCG
SDSGQGAPPHGSLEKGNSTTLD
NOV14p,
CG51051-08 SEQ ID NO: 191 1837 bp
DNA Sequence ORF Start: ATG at 230 ORF Stop TAG at 1610
GGCTTCCACCAAAGTCCTCAATATACCTGAATACGCACAATATCTTAACTCTTCATATTTGGTTTTGG
GATCTGCTTTGAGGTCCCATCTTCATTTAAAAAAAAATACAGACACCTACCTACCCGTACGCATACAT
ACATATGTGTATATATATGTAAACTAGACAAAGATCGCAGATCATAAAGCAAGCTCTGCTTTAGTTTC
CAAGAAGATTACAAAGAATTTAGAGATGTATTTGTCAAGATTCCTGTCGATTCATGCCCTTTGGGTTA
CGGTGTCCTCAGTGATGCAGCCCTACCCTTTGGTTTGGGGACATTATGATTTGTGTAAGACTCAGATT
TACACGGAAGAAGGGAAAGTTTGGGATTACATGGCCTGCCAGCCGGAATCCACGGACATGACAAAATA
TCTGAAAGTGAAACTCGATCCTCCGGATATTACCTGTGGAGACCCTCCTGAGACGTTCTGTGCAATGG
GCAATCCCTACATGTGCAATAATGAGTGTGATGCGAGTACCCCTGAGCTGGCACACCCCCCTGAGCTG
ATGTTTGATTTTGAAGGAAGACATCCCTCCACATTTTGGCAGTCTGCCACTTGGAAGGAGTATCCCAA
GCCTCTCCACGTTAACATCACTCTGTCTTGCAGCAAAACCATTGAGCTAACAGACAACATAGTTATTA
CCTTTGAATCTGGGCGTCCAGACCAAATGATCCTGGAGAAGTCTCTCGATTATGGACGAACATGGCAG
CCCTATCAGTATTATGCCACAGACTGCTTAGATGCTTTTCACATGGATCCTAAATCCGTGAAGGATTT
ATCACAGCATACGGTCTTAGAAATCATTTGCACAGAAGAGTACTCAACAGGGTATACAACAAATAGCA
AAATAATCCACTTTGAAATCAAAGACAGGTTCGCGTTTTTTGCTGGACCTCGCCTACGCAATATGGCT
TCCCTCTACGGACAGCTGGATACAACCAAGAAACTCAGAGATTTCTTTACAGTCACAGACCTGAGGAT
AAGGCTGTTAAGACCAGCCGTTGGGGAAATATTTGTAGATGAGCTACACTTGGCACGCTACTTTTACG
CGATCTCAGACATAAAGGTGCGAGGAAGGTGCAAGTGTAATCTCCATGCCACTGTATGTGTGTATGAC
AACAGCAAATTGACATGCGAATGTGAGCACAACACTACAGGTCCAGACTGTGGGAAATGCAAGAAGAA
TTATCAGGGCCGACCTTGGAGTCCAGGCTCCTATCTCCCCATCCCCAAAGGCACTGCAAATACCTGTA
TCCCCAGTATTTCCAGTATTGGTAATCCTCCAAAGTTTAATAGGATATGGCCGAATATTTCTTCCCTT
GAGGTTTCTAACCCAAAACAAGCGAATGTCTGCGACAACGAGCTCCTGCACTGCCAGAACGGAGGGAC
GTGCCACAACAACCTGCGCTGCCTGTGCCCGGCCGCATACACGGGCATCCTCTGCGACAAGCTGCGGT
GCGAGGAGGCTGGCAGCTGCGGCTCCGACTCTGGCCAGGGCGCGCCCCCGCACGGCTCCCCAGCGCTG
CTGCTGCTGACCACGCTGCTGGGAACCGCCAGCCCCCTGGTGTTCTAGGTGTCACCTCCAGCCACACC
GGACGGGCCTGTGCCGTGGGGAAGCAGACACAACCCAAACATTTGCTACTAACATAGGAAACACACAC
ATACAGACACCCCCACTCAGACAGTGTACAAACTAAGAAGGCCTAACTGAACTAAGCCATATTTATCA
CCCGTGGACAGCACATCCGAGTCAAGACTGTTAATTTCTGACTCCAGAGGAGTTGGCAGCTGTTGATA
T
NOV14p,
CG51051-08
Protein Sequence SEQ ID NO: 192 460 aa 51857.6 kD
MYLSRFLSIHALWVTVSSVMQPYPLVWGHYDLCKTQIYTEEGKVWDYMACQPESTDMTKYLKVKLDPP
DITCGDPPETFCAMGNPYMCNNECDASTPELAHPPELMFDFEGRHPSTFWQSATWKEYPKPLQVNITL
SWSKTIELTDNIVITFESGRPDQNILEKSLDYGRTWQPYQYYATDCLDAFHMDPKSVAALSQHTVLEI
ICTEEYSTGYTTNSKIIHFEIKDRFAFFAGPRLRNMASLYGQLDTTKKLRDFFTVTDLRIRLLRPAVG
EIFVDELHLARYFYAISDIKVRGRCKCNLHATVCVYDNSKLTCECEHNTTGPDCGKCKAAYQGRPWSP
GSYLPIPKGTANTCIPSISSIGNPPKFNRIWPNISSLEVSNPKQAAVCDNELLHCQNGGTCHNNVRCL
CPAAYTGILCEKLRCEEAGSCGSDSGQGAPPHGSPALLLLTTLLGTASPLVF
NOV14q,
CG51051-10 SEQ ID NO: 193 426 bp
DNA Sequence ORF Start: at 29 ORF Stop: end of sequence
AGGCTCCGCGGCCGCCCCCTTCACCGTGATGCAGCCCTACCCTTTOGTTTGGGGACATTATGATTTGT
GTAGACTCAGATTTACACGGAAGAAGGGAAAAGTTTGGGATTACATGGCCTGCCAGCCGGAATCCACG
GACATGACAAAATATCTGAAAGTGAAACTCGATCCTCCGGATATTACCTGTGGAGACCCTCCTGAGAC
GTTCTGTGCAATGGGCAATCCCTACATGTGCAATAATGAGTGTGATGCGAGTACCCCTGAGCTGGCAC
ACCCCCCTGAGCTGATGTTTGATTTTGAAGGAAGACATCCCTCCACATTTTGGCAGTCTGCCACTTGG
AAGGAGTATCCCAAGCCTCTCCAGGTTAACATCACTCTGTCTTGGAGCAAAACCATTGAGCTAACAGA
CAACATAGTTATTACCTTTGAATCTGGGCGTCCAGACCAAATGATCCTGGAGAAGTCTCTCGATTATG
GACGAACATGGCAGCCCTATCAGTATTATGCCACAGACTGCTTAGATGCTTTTCACATGGATCCTAAA
TCCGTGAAGGATTTATCACAGCATACGGTCTTAGAAATCATTTGCACAGAAGAGTACTCAACAGGGTA
TACAACAAATAGCAAAATAATCCACTTTGAAATCAAAGACAGGTTCGCGTTTTTTGCTGGACCTCGCC
TACGCAATATGGCTTCCCTCTACGGACAGCTGGATACAACCAAGAAACTCAGAGATTTCTTTACAGTC
ACAGACCTGAGGATAAGGCTGTTAAGACCAGCCGTTGGGGAAATATTTGTAGATGAGCTACACTTGGC
ACGCTACTTTTACGCGATCTCAGACATAAAGGTGCGAGGAAGGTGCAAGTGTAATCTCCATGCCACTG
TATGTGTGTATGACAACAGCAAATTGACATGCGAATGTGAGCACAACACTACAGGTCCAGACTGTGGG
AAATGCAAGAAGAATTATCAGGGCCGACCTTGGAGTCCAGGCTCCTATCTCCCCATCCCCAAAGGCAC
TGCAAATACCTGTATCCCCAGTATTTCCAGTATTGGTAATCCTCCAAAGTTTAATAGGATATGGCCGA
ATATTTCTTCCCTTGAGGTTTCTAACCCAAAACAAGTTGCTCCCAAATTAGCTTTGTCAACAGTTTCT
TCTGTTCAAGTTGCAAACCACAAGAGACCGAATGTCTGCGACAACGAGCTCCTGCACTGCCAGAACGG
AGGGACGTGCCACAACAACGTGCGCTCCCTGTGCCCGGCCGCATACACGGGCATCCTCTGCGAGAAGC
TGCCGTGCGAGGAGGCTGGCAGCTGCCGCTCCGACTCTGGCCAGGGCGCGCCCCCGCACGGCTCCCCA
GCGCTGCTGCTGCTGACCACGCTGCTGGGAACCGCCAGCCCCCTGGTGTTCAAGGGTGGGCGCGCC
NOV14q,
CG51051-10
Protein Sequence SEQ ID NO: 194 462 aa MW at 51852.6 kD
VMQPYPLVWGHYDLCKTQIYTEEGKVWDYMACQPESTDMTKYLKVKLDPPDITCGDPPETFCAMGNPY
MCNNECDASTPELAHPPELMFDFEGRHPSTFWQSATWKEYPKPLQVNITLSWSKTIELTDNIVITFES
GRPDQMILEKSLDYGRTWQPYQYYATDCLDAFHMDPKSVKDLSQHTVLEIICTEEYSTGYTTNSKIIH
FEIKDRFAFFAGPRLRNMASLYGQLDTTKKLRDFFTVTDLRIRLLRPAVGEIFVDELHLARYFYAISD
IKVRGRCKCNLHATVCVYDNSKLTCECEHNTTGPDCGKCKKNYQGRPWSPGSYLPIPKGTANTCIPSI
SSIGNPPKFMRIWPNISSLEVSNPKQVAPKLALSTVSSVQVANHKRANVCDNELLHCQNGGTCHNNVR
CLCPAAYTGILCEKLRCEEAGSCGSDSGQGAPPHGSPALLLLTTLLGTASPLVF
NOV14r,
CG51051-11 SEQ ID NO: 195 778 bp
DNA Sequence ORF Start: at 2 ORF Stop: end of sequence
GGCTCCGCGGCCGCCCCCTTCACCTGCCAGCCGGAATCCACGGACATGACAAAATATCTGAAAGTGA
AACTCGATCCTCCGGATATTACCTGTGGAGACCCTCCTGAGACGTTCTGTGCAATGGGCAATCCCTAC
ATGTGCAATAATGAGTGTGATGCGAGTACCCCTGAGCTGGCACACCCCCCTGAGCTGATGTTTGATTT
TGAAGGAAGACATCCCTCCACATTTTGGCAGTCTGCCACTTGGAAGGAGTATCCCAAGCCTCTCCAGG
TTAACATCACTCTGTCTTGGAGCAAAACCATTGAGCTAACAGACAACATAGTTATTACCTTTGAATCT
GGGCGTCCAGACCAAATGATCCTGGAGAAGTCTCTCGATTATGGACGAACATGGCAGCCCTATCAGTA
TTATGCCACAGACTGCTTAGATGCTTTTCACATGGATCCTAAATCCGTGAAGGATTTATCACAGCATA
CGGTCTTAGAAATCATTTGCACAGAAGAGTACTCAACAGGGTATACAACAAATAGCAAAATAATCCAC
TTTGAAATCAAAGACAGGTTCGCGTTTTTTGCTGGACCTCGCCTACGCAATATGGCTTCCCTCTACGG
ACAGCTGGATACAACCAAGAAACTCAGAGATTTCTTTACAGTCACAGACCTGAGGATAAGGCTGTTAA
GACCAGCCGTTGGGGAAATATTTGTAGATGAGCTACACTTGGCACGCTACTTTTACGCGATCTCAGAC
ATAAAGGTGCGAGGAAAGGGTGGGCGCGCC
NOV14r,
CG51051-11
Protein Sequence SEQ ID NO: 196 1259 aa MW at 29645.3 kD
GSAAPFTCQPESTDMTKYLKVKLDPPDITCGDPPETFCAMGNPYMCNNECDASTPELAAAPPELMFDF
EGRHPSTFWQSATWKEYPKPLQVNITLSWSKTIELTDNIVITFESGRPDQMILEKSLDYGRTWQPYQY
YATDCLDAFHMDPKSVKDLSQUTVLEIICTEEYSTGYTTNSKIIHFEIKDRFAFFAGPRLRNMASLYG
QLDTTKKLRDFFTVTDLRIRLLRPAVGEIFVDELHLARYFYAISDIKVRGKGGRA
NOV14s,
CG51051-12 SEQ ID NO: 197 1452 bp
DNA Sequence ORF Start: ATG at 7 ORF Stop: end of sequence
AGATCTATGTATTTGTCAAGATTCCTGTCGATTCATGCCCTTTGGGTTACGGTGTCCTCAGTGATGCA
GCCCTACCCTTTGGTTTGGGGACATTATGATTTGTGTAAGACTCAGATTTACACGGAAGAAGGGAAAG
TTTGGGATTACATGGCCTGCCAGCCGGAATCCACGGACATGACAAATATCTGAAAAAGTGAACTCGAT
CCTCCGGATATTACCTGTGGAGACCCTCCTGAGACGTTCTGTGCAATGCGCAATCCCTACATGTGCAA
TAATGAGTGTGATGCGAGTACCCCTGAGCTGGCACACCCCCCTGAGCTGATGTTTGATTTTGAAGGAA
GACATCCCTCCACATTTTGGCAGTCTGCCACTTGGAAGGAGTATCCCAAGCCTCTCCAGGTTAACATC
ACTCTGTCTTGGAGCAAAACCATTGAGCTAACAGACAACATAGTTATTACCTTTGAATCTGGGCGTCC
AGACCAAATGATCCTGGAGAAGTCTCTCGATTATGGACGAACATGGCAGCCCTATCAGTATTATGCCA
CAGACTGCTTAGATGCTTTTCACATGGATCCTAAATCCGTGAAGGATTTATCACAGCATACGGTCTTA
GAAATCATTTGCACAGAAGAGTACTCAACAGGGTATACAACAAATAGCAAAATAATCCACTTTGAAAT
CAAAGACAGGTTCGCGTTTTTTGCTGGACCTCGCCTACGCAATATGGCTTCCCTCTACGGACAGCTGG
ATACAACCAAGAAACTCAGAGATTTCTTTACAGTCACAGACCTGAGGATAAGGCTGTTAAGACCAGCC
GTTGGGGAAATATTTGTAGATGAGCTACACTTGGCACGCTACTTTTACGCGATCTCAGACATAAAGGT
GCGAGGAAGGTGCAAGTGTAATCTCCATGCCACTGTATGTGTGTATGACAACAGCAAATTGACATGCG
AATGTGAGCACAACACTACAGGTCCAGACTGTGGGAAATGCAAGAAGAATTATCAGGGCCGACCTTGG
AGTCCAGGCTCCTATCTCCCCATCCCCAAAGGCACTGCAAATACCTGTATCCCCAGTATTTCCAGTAT
TGGTAATCCTCCAAAGTTTAATAGGATATGGCCGAATATTTCTTCCCTTGAGGTTTCTAACCCAAAAC
AAGTTGCTCCCAAATTAGCTTTGTCAACAGTTTCTTCTGTTCAAGTTGCAAACCACAAGAGAGCGAAT
GTCTGCGACAACGAGCTCCTGCACTGCCAGAACGGAGGGACGTGCCACAACAACGTGCGCTGCCTGTG
CCCGGCCGCATACACGGGCATCCTCTGCGAGAAGCTGCGGTGCGAGGAGGCTGGCAGCTGCGGCTCCG
ACTCTGGCCAGGGCGCGCCCCCGCACGGCTCCCCAGCGCTGCTGCTGCTGACCACGCTGCTGGGAACC
GCCAGCCCCCTGGTGTTC
NOV14s,
CG51051-12
Protein Sequence SEQ ID NO: 198 480 aa MW at 53945.0 kD
MYLSRFLSIHALWVTVSSVMQPYPLVWGHYDLCKTQIYTEEGKAADYAACQPESTDMTKYLAAKLDPP
DITCGDPPETFCAMGNPYMCNNECDASTPELAHPPELMFDFEGRHPSTFWQSATWKEYPKPLQVNITL
SWSKTIELTDNIVITFESGRPDQMILEKSLDYGRTWQPYQYYATDCLDAFHMDPKSVAALSQHTAAEI
ICTEEYSTGYTTNSKIIHFEIKDRFAFFAGPRLRNMASLYGQLDTTKKLRDFFTAADLRIRLLRPAVG
EIFVDELHLARYFYAISDIKVRGRCKCNLHATVCVYDNSKLTCECEHNTTGPDCGKCKKNYQGRPWSP
GSYLPIPKGTANTCIPSISSIGNPPKFNRIWPNISSLEVSNPKQVAPKLALSTVSSVQVANHKRANVC
DNELLHCQNGGTCHNNVRCLCPAAYTGILCEKLRCEEAGSCGSDSGQGAPPHGSPALLLLTTLLGTAS
PLVF
NOV14t,
CG51051-15 SEQ ID NO: 199 750 bp
DNA Sequence ORF Start: at 1 ORF Stop: end of sequence
TGCCAGCCGGAATCCACGGACATGACAAAATATCTGAAAGTGAAACTCGATCCTCCGGATAT
TACCTGTGGAGACCCTCCTGAGACGTTCTGTGCAATGGGCAATCCCTACATGTGCAATAATGAGTGTG
ATGCGAGTACCCCTGAGCTGGCACACCCCCCTGAGCTGATGTTTGATTTTGAAGGAAGACATCCCTCC
ACATTTTGGCAOTCTGCCACTTGGAAGGAGTATCCCAAGCCTCTCCAGGTTAACATCACTCTGTCTTG
GAGCAAAACCATTGAGCTAACAGACAACATAGTTATTACCTTTGAATCTGGGCGTCCAGACCAAATGA
TCCTGGAGAAGTCTCTCGATTATGGACGAACATGGCAGCCCTATCAGTATTATGCCACAGACTGCTTA
GATGCTTTTCACATGGATCCTAAATCCGTGAAGGATTTATCACAGCATACGGTCTTAGAAATCATTTG
CACAGAAGAGTACTCAACAGGGTATACAACAAATAGCAAAATAATCCACTTTGAAATCAAAGACAGGT
TCGCGTTTTTTGCTGGACCTCGCCTACGCAATATGGCTTCCCTCTACGGACAGCTGGATACAACCAAG
AAACTCAGAGATTTCTTTACAGTCACAGACCTGAGGATAAGGCTGTTAAGACCAGCCGTTGGGGAAAT
ATTTGTAGATGAGCTACACTTGGCACGCTACTTTTACGCGATCTCAGACATAAAGGTGCGAGGA
NOV14t,
CG51051-13
Protein Sequence SEQ ID NO: 200 246 aa MW at 28473.0 kD
CQPESTDMTKYLKVKLDPPDITCGDPPETFCANGNPYMCNNECDASTPELAHPPELMFDFEGRHPSTF
WQSATWKEYPKPLQVNITLSWSKTIELTDNIVITFESGRPDQMILEKSLDYGRTWQPYQYYATDCLDA
FHMDPKSVKDLSQHTVLEIICTEEYSTGYTTNSKIIHFEIKDRFAFFAGPRLRNMASLYGQLDTTKKL
RDFFTVTDLRIRLLRPAVGEIFVDELHLARYFYAISDIKVRG
NOV14u,
CG51051-15 SEQ ID NO: 201 1465 bp
DNA Sequence ORF Start: ATG at 17 ORF Stop: end of sequence
CACCAGATCTCCCACCATGTATTTGTCAAGATTCCTGTCGATTCATGCCCTTTGGGTTACGGTGTCCT
CAGTGATGCAGCCCTACCCTTTGGTTTGGGGACATTATGATTTGTGTAAGACTCAGATTTACACGGAA
GAAGGGAAAGTTTGGGATTACATGGCCTGCCAGCCGGAATCCACGGACATGACAAAATATCTGAAAGT
GAAACTCGATCCTCCGGATATTACCTGTGGAGACCCTCCTGAGACGTTCTGTGCAATGGGCAATCCCT
ACATGTGCAATAATGAGTGTGATGCGAGTACCCCTGAGCTGGCACACCCCCCTGAGCTGATGTTTGAT
TTTGAAGGAAGACATCCCTCCACATTTTGGCAGTCTGCCACTTGGAAGGAGTATCCCAAGCCTCTCCA
GGTTAACATCACTCTGTCTTGGAGCAAAACCATTGAGCTAACAGACAACATAGTTATTACCTTTGAAT
CTGGGCGTCCAGACCAAATGATCCTGGAGAAGTCTCTCGATTATGGACGAACATGGCAGCCCTATCAG
TATTATGCCACAGACTGCTTAGATGCTTTTCACATGGATCCTAAATCCGTGAAGGATTTATCACAGCA
TACGGTCTTAGAAATCATTTGCACAGAAGAGTACTCAACAGGGTATACAACAAATAGCAAAATAATCC
ACTTTGAAATCAAAGACAGGTTCGCGTTTTTTGCTGGACCTCGCCTACGCAATATGGCTTCCCTCTAC
CGACAGCTGGATACAACCAAGAAACTCAGAGATTTCTTTACAGTCACAGACCTGAGGATAAGGCTGTT
AAGACCAGCCGTTGGGGAAATATTTGTAGATGAGCTACACTTGGCACGCTACTTTTACGCGATCTCAG
ACATAAAGGTGCGAGGAAGGTGCAAGTGTAATCTCCATGCCACTGTATGTGTGTATGACAACAGCAAA
TTGACATCCGAATGTGAGCACAACACTACAGGTCCAGACTGTGGGAAATGCAAGAAGAATTATCAGGG
CCGACCTTGGAGTCCACGCTCCTATCTCCCCATCCCCAAAGGCACTGCAAATACCTGTATCCCCAGTA
TTTCCAGTATTGGTAATCCTCCAAAGTTTAATAGGATATGGCCGAATATTTCTTCCCTTGAGGTTTCT
AACCCAAAACAAGTTGCTCCCAAATTAGCTTTGTCAACAGTTTCTTCTGTTCAAGTTGCAAACCACAA
GAGAGCGAATGTCTGCGACAACGAGCTCCTGCACTGCCAGAACGGAGGGACGTGCCACAACAACGTGC
GCTGCCTGTGCCCGGCCGCATACACGGGCATCCTCTGCGAGAAGCTCCGGTGCGAGGAGGCTGGCAGC
TGCGGCTCCGACTCTGCCCAGGGCGCGCCCCCGCACGGCTCCCCAGCGCTUCTGCTGCTGACCACGCT
GCTGGGAACCGCCAGCCCCCTGGTGTTC
NOV14u,
CG51051-15
Protein Sequence SEQ ID NO: 202 480 aa MW at 53945.0 kD
MYLSRFLSIHALWVTVSSVMQPYPLVWGHYDLCKTQIYTEEGKVWDYMACQPESTDMTKYLKVKLDPP
DITCGDPPETFCAMGNPYMCNNECDASTPELAHPPELMFDFEGRHPSTFWQSATWKEYPKPLQVNITL
SWSKTIELTDNIVITFESGRPDQMILEKSLDYGRTWQPYQYYATDCLDAFHMDPKSVKDLSQHTVLEI
ICTEEYSTGYTTNSKIIHFEIKDRFAFFAGPRLRNMASLYGQLDTTKKLRDFFTVTDLRIRLLRPAVG
EIFVDELHLARYFYAISDIKVRGRCKCNLHATVCVYDNSKLTCECEHNTTGPDCGKCKKNYQGRPWSP
GSYLPIPKGTANTCIPSISSIGNPPKFNRIWPNISSLEVSNPKQVAPKLALSTVSSVQVANHKRANVC
DNELLHCQNGGTCHNNVRCLCPAAYTGILCEKLRCEEAGSCGSDSGQGAPPHGSPALLLLTTLLGTAS
PLVF
NOV14v,
CG51051-16 SEQ ID NO: 203 1467 bp
DNA Sequence ORF Start: ATG at 16 ORF Stop: TAG at 1456
CACCGCGGCCGCACCATGTATTTGTCAAGATTCCTGTCGATTCATGCCCTTTGGGTTACGGTGTCCTC
AGTGATGCAGCCCTACCCTTTGGTTTGGCCACATTATGATTTGTGTAAGACTCAGATTTACACGGAAG
AAGGGAAAGTTTGGGATTACATGGCCTGCCAGCCGGAATCCACGGACATGACAAAATATCTGAAAGTC
AAACTCGATCCTCCGGATATTACCTGTGOAGACCCTCCTGAGACGTTCTGTGCAATGGGCAATCCCTA
CATGTCCAATAATGAGTGTGATGCGAGTACCCCTGAGCTGGCACACCCCCCTGAGCTGATGTTTGATT
TTGAAGGAAGACATCCCTCCACATTTTGGCAGTCTGCCACTTGGAAGGAGTATCCCAAGCCTCTCCAG
GTTAACATCACTCTGTCTTGGAGCAAAACCATTGAGCTAACAGACAACATAGTTATTACCTTTGAATC
TGGGCGTCCAGACCAAATGATCCTGGACAAGTCTCTCGATTATGGACGAACATGGCAGCCCTATCAGT
ATTATGCCACAGACTGCTTAGATGCTTTTCACATGGATCCTAAATCCGTGAAGGATTTATCACAGCAT
ACGGTCTTAGAAATCATTTGCACAGAAGAGTACTCAACAGGGTATACAACAAATAGCAAAATAATCCA
CTTTGAAATCAAAGACAGGTTCGCGTTTTTTGCTGGACCTCGCCTACGCAATATGCCTTCCCTCTACG
GACAGCTGGATACAACCAAGAAACTCAGAGATTTCTTTACAGTCACAGACCTGAGCATAAGGCTGTTA
AGACCAGCCGTTGGGGAAATATTTGTAGATGAGCTACACTTGGCACGCTACTTTTACGCGATCTCAGA
CATAAAGGTGCGAGGAAGGTGCAAGTGTAATCTCCATGCCACTGTATGTGTGTATGACAACAGCAAAT
TGACATGCGAATGTGAGCACAACACTACAGGTCCAGACTGTGGGAAATGCAACAAGAATTATCAGGGC
CGACCTTGGAGTCCAGGCTCCTATCTCCCCATCCCCAAAGGCACTGCAAATACCTGTATCCCCAGTAT
TTCCAGTATTGGTAATCCTCCAAAGTTTAATAGGATATGGCCGAATATTTCTTCCCTTGAGGTTTCTA
ACCCAAAACAAGTTGCTCCCAAATTAGCTTTGTCAACAGTTTCTTCTGTTCAAGTTGCAAACCACAAG
AGAGCGAATGTCTGCGACAACGAGCTCCTGCACTGCCAGAACGGAGGGACGTGCCACAACAACGTGCG
CTGCCTGTGCCCGGCCGCATACACGGGCATCCTCTGCGAGAAGCTGCGGTGCGAGGAGGCTGGCAGCT
GCGGCTCCGACTCTGGCCACGGCGCGCCCCCGCACGGCTCCCCAGCGCTGCTGCTGCTGACCACGCTG
CTGGGAACCGCCAGCCCCCTGGTGTTCTAGGTCGACGGC
NOV14v,
CG51051-16
Protein Sequence SEQ ID NO: 204 480 aa MW at 53945.0 kD
MYLSRFLSIHALWVTVSSVMQPYPLVWGHYDLCKTQIYTEEGKVWDYMACQPESTDMTKYLKVKLDPP
DITCGDPPETFCAMGNPYMCNNECDASTPELAHPPELMFDFEGRHPSTFWQSATWKEYPKPLQVNITL
SWSKTIELTDNIVITFESGRPDQMILEKSLDYGRTWQPYQYYATDCLDAFHMDPKSVKDLSQHTVLEI
ICTEEYSTGYTTNSKIIHFEIKDRFAFFAGPRLRNMASLYGQLDTTKKLRDFFTVTDLRIRLLRPAVG
EIFVDELHLARYFYAISDIKVRGRCKCNLHATVCVYDNSKLTCECEHNTTGPDCGKCKKNYQGRPWSP
GEYLPIPKGTANTCIPSISSIGNPPKFNRIWPNISSLEVSNPKQVAPKLALSTVSSVQVANHKRANVC
DNELLHCQNGGTCHNNVRCLCPAAYTGILCEKLRCEEAGSCGSDSGQGAPPHGSPALLLLTTLLGTAS
PLVF
NOV14w,
13380736 SNP
for CG51051-07 SEQ ID NO: 205 1495 bp, SNP: 217 A/G
DNA Sequence ORF Start: ATG at 46 ORF Stop: TAG at 1486
TCAAGCTCTGCTTTAGTTTCCAAGAAGATTACAAAGAATTTAGAGATGTATTTGTCAAGATTCCTGTCGAT
TCATGCCCTTTGGGTTACGGTGTCCTCAGTGATGCAGCCCTACCCTTTGGTTTGGGGACATTATGATTTGT
GTAAGACTCAGATTTACACGGAAGAAGGGAAAGTTTGGGATTACATGGCCTGCCAGCCGGAATCCACGGAC
ATGGCAAAATATCTGAAAGTGAAACTCGATCCTCCGGATATTACCTGTGGAGACCCTCCTGAGACGTTCTG
TGCAATGGGCAATCCCTACATGTGCAATAATGAGTGTGATGCGAGTACCCCTGAGCTGGCACACCCCCCTG
AGCTGATGTTTGATTTTGAAGGAAGACATCCCTCCACATTTTGGCAGTCTGCCACTTGGAAGGAGTATCCC
AAGCCTCTCCAGGTTAACATCACTCTGTCTTGGAGCAAAACCATTGAGCTAACAGACAACATAGTTATTAC
CTTTGAATCTGGGCGTCCAGACCAAATGATCCTGGAGAAGTCTCTCGATTATGGACGAACATGGCAGCCCT
ATCAGTATTATGCCACAGACTGCTTAGATGCTTTTCACATGGATCCTAAATCCGTGAAGGATTTATCACAG
CATACGGTCTTAGAAATCATTTGCACAGAAGAGTACTCAACAGGGTATACAACAAATAGCAAAATAATCCA
CTTTGAAATCAAAGACAGGTTCGCGTTTTTTGCTGGACCTCGCCTACGCAATATGGCTTCCCTCTACGGAC
AGCTGGATACAACCAAGAAACTCAGAGATTTCTTTACAGTCACAGACCTGAGGATAAGGCTGTTAAGACCA
GCCGTTGGGGAAATATTTGTAGATGAGCTACACTTGGCACGCTACTTTTACGCGATCTCAGACATAAAGGT
GCGAGGAAGGTGCAAGTGTAATCTCCATGCCACTGTATGTGTGTATGACAACAGCAAATTGACATGCGAAT
GTGAGCACAACACTACAGGTCCAGACTGTGGGAAATGCAAGAAGAATTATCAGGGCCGACCTTGGAGTCCA
GGCTCCTATCTCCCCATCCCCAAAGGCACTGCAAATACCTGTATCCCCAGTATTTCCAGTATTGGTAATCC
TCCAAAGTTTAATAGGATATGGCCGAATATTTCTTCCCTTGAGGTTTCTAACCCAAAACAAGTTGCTCCCA
AATTAGCTTTGTCAACAGTTTCTTCTGTTCAAGTTGCAAACCACAAGAGAGCGAATGTCTGCGACAACGAG
CTCCTGCACTGCCAGAACGGAGGGACGTGCCACAACAACGTGCGCTGCCTGTGCCCGGCCGCATACACGGG
CATCCTCTGCGAGAAGCTGCGGTGCGAGGAGGCTGGCAGCTGCGGCTCCGACTCTGGCCAGGGCGCGCCCC
CCCACGGCTCCCCAGCCCTGCTGCTGCTGACCACGCTGCTGGGAACCGCCAGCCCCCTGGTGTTCTAGGTG
TCAC
NOV14w,
13380736 SNP
for CG51051-07 SEQ ID NO: 206 480 aa SNP: Thr to Ala
Protein Sequence at position 58
MYLSRFLSINALWVTVSSVMQPYPLVWGNYDLCKTQIYTEECKAADYAACQPESTDAAKYLKVKLDPPDIT
CGDPPETFCAAGNPYMCNNECDASTPELAAPPELMFDFEGRHPSTFWQSATWKEYPKPLQAAITLSWSKTI
ELTDNIVITFESGRPDQMILEKSLDYGRTWQPYQYYATDCLDAFHMDPKSVAALSQHTAAEIICTEEYSTG
YTTNSKHHFEIKDRFAFFAGPRLRNMASLYGQLDTTKKLRDFFTVAADLRIRLLRPAVGEIFVDELHLARY
FYAISDIAARGRCKCNLHATVCAADNSKLTCECEHNTTGPDCGKCKAAYQGRPWSPGSYLPIPKGTAATCI
PSISSIGNPPKFNRIWPNISSLEVSNPKQVAPKLALSTVSSVQVANHKRANVCDNELLHCQNGGTCHNNVR
CLCPAAYTGILCEKLRCEEAGSCGSDSGQGAPPHGSPALLLLTTLLGTASPLVF
NOV14x,
13380734 SNP
for CG51051007 SEQ ID NO: 207 1495 bp, SNP: 872 T/G
DNA Sequence ORF Start: ATG at 46 ORF Stop: TAG at 1486
TCAAGCTCTGCTTTAGTTTCCAAGAAGATTACAAAGAATTTAGAGATGTATTTGTCAAGATTCCTGTCGAT
TCATGCCCTTTGGGTTACGGTGTCCTCAGTGATGCAGCCCTACCCTTTGGTTTGGGGACATTATGATTTGT
GTAAGACTCAGATTTACACGGAAGAAGGGAAAGTTTGGGATTACATGGCCTGCCAGCCGGAATCCACGGAC
ATGACAAAATATCTGAAAGTGAAACTCGATCCTCCGGATATTACCTGTGGAGACCCTCCTGAGACGTTCTG
TGCAATGGGCAATCCCTACATGTGCAATAATGAGTGTGATGCGAGTACCCCTGAGCTGGCACACCCCCCTG
AGCTGATGTTTGATTTTGAAGGAAGACATCCCTCCACATTTTGGCAGTCTGCCACTTGGAAGGAGTATCCC
AAGCCTCTCCAGGTTAACATCACTCTGTCTTGGAGCAAAACCATTGAGCTAACAGACAACATAGTTATTAC
CTTTGAATCTGGGCGTCCAGACCAAATGATCCTGGAGAAGTCTCTCGATTATGGACGAACATGGCAGCCCT
ATCAGTATTATGCCACAGACTGCTTAGATGCTTTTCACATGGATCCTAAATCCGTGAAGGATTTATCACAG
CATACGGTCTTAGAAATCATTTGCACAGAAGAGTACTCAACAGGGTATACAACAAATAGCAAAATAATCCA
CTTTGAAATCAAAGACACGTTCGCGTTTTTTGCTGGACCTCGCCTACGCAATATGGCTTCCCTCTACGGAC
AGCTGGATACAACCAAGAAACTCAGAGATTTCTTTACAGTCACAGACCTGAGGATAAGGCTGTTAAGACCA
GCCGTTGGGGAAATATTTGGAGATGAGCTACACTTGGCACGCTACTTTTACGCGATCTCAGACATAAAGGT
GCGAGGAAGGTGCAAGTGTAATCTCCATGCCACTGTATGTGTGTATGACAACAGCAAATTGACATGCGAAT
GTGAGCACAACACTACAGGTCCAGACTGTGGGAAATGCAAGAAGAATTATCAGGGCCGACCTTGGAGTCCA
GGCTCCTATCTCCCCATCCCCAAAGCCACTGCAAATACCTGTATCCCCAGTATTTCCAGTATTGGTAATCC
TCCAAAGTTTAATAGGATATGGCCGAATATTTCTTCCCTTGAGGTTTCTAACCCAAAACAAGTTGCTCCCA
AATTAGCTTTGTCAACAGTTTCTTCTGTTCAAGTTGCAAACCACAAGAGACCGAATGTCTGCGACAACGAG
CTCCTGCACTGCCAGAACGGAGGGACGTGCCACAACAACGTGCGCTGCCTGTGCCCGGCCGCATACACGGG
CATCCTCTGCGAGAAGCTGCGGTGCGAGGAGGCTGGCAGCTGCGGCTCCGACTCTGGCCAGGGCGCGCCCC
CGCACGGCTCCCCAGCGCTGCTGCTGCTGACCACCCTGCTGGGAACCGCCAGCCCCCTGGTGTTCTAGGTG
TCAC
NOV14x,
13380734 SNP
for CG51051-07 SEQ ID NO: 208 480 aa SNP: Val to Gly
Protein Sequence at position 276
MYLSRFLSIHALWVTVSSVMQPYPLVWGHYDLCKTQIYTEEGKVWDYMACQPESTDMTKYLKVKLDPPDIT
CGDPPETFCAMGNPYMCNNECDASTPELAAPPELMFDFEGRMPSTFWQSATWKEYPKPLQVNITLSWSKTI
ELTDNIVITFESGRPDQMTLEKSLDYGRTWQPYQYYATDCLDAFHMDPKSVKDLSQHTVLEIICTEEYSTG
YTTNSKIIHFEIKDRFAFFAGPRLRNMASLYGQLDTTKKLRDFFTAADLRIRLLRPAVGEIFGDELHLARY
FYAISDIKVRGRCKCNLHATVCVYDNSKLTCECEHNTTGPDCGKCKAAYQGRPWSPGSYLPIPKGTANTCI
PSISSIGNPPKFNRIWPNISSLEVSNPKQVAPKLALSTVSSVQVANHKRANVCDNELLHCQNGGTCHNNVR
CLCPAAYTGILCEKLRCEEAGSCGSDSGQGAPPHGSPALLLLTTLLGTASPLVF
NOV14y,
13382329 SNP
for CG51051-07 SEQ ID NO: 209 1495 bp SNP: 1448 T/A
DNA Sequence ORF Start: ATG at 46 ORF Stop: TAG at 1486
TCAAGCTCTGCTTTAGTTTCCAAGAAGATTACAAAGAATTTAGAGATGTATTTGTCAAGATTCCTGTCGAG
TCATGCCCTTTGGGTTACGGTGTCCTCAGTGATGCAGCCCTACCCTTTGGTTTGGGGACATTATGATTTGT
GTAAGACTCAGATTTACACGGAAGAAGGGAAAGTTTGGGATTACATGGCCTGCCAGCCGGAATCCACGGAC
ATGACAAAATATCTGAAAGTGAAACTCGATCCTCCGGATATTACCTGTGGAGACCCTCCTGAGACGTTCTG
TGCAATGGGCAATCCCTACATGTGCAATAATGAGTGTGATGCGAGTACCCCTGAGCTGGCACACCCCCCTG
AGCTGATGTTTGATTTTGAAGGAAGACATCCCTCCACATTTTGGCAGTCTGCCACTTGGAAGGAGTATCCC
AAGCCTCTCCAGGTTAACATCACTCTGTCTTGGAGCAAAACCATTGAGCTAACAGACAACATAGTTATTAC
CTTTGAATCTGGGCGTCCAGACCAAATGATCCTGGAGAAGTCTCTCGATTATGGACGAACATGGCAGCCCT
ATCAGTATTATGCCACAGACTGCTTAGATGCTTTTCACATGGATCCTAAATCCGTGAAGGATTTATCACAG
CATACGGTCTTAGAAATCATTTGCACAGAAGAGTACTCAACAGGGTATACAACAAATAGCAAAATAATCCA
CTTTGAAATCAAAGACAGGTTCGCGTTTTTTGCTGGACCTCGCCTACGCAATATGGCTTCCCTCTACGGAC
AGCTGGATACAACCAAGAAACTCAGAGATTTCTTTACAGTCACAGACCTGAGGATAAGGCTGTTAAGACCA
GCCGTTGGGGAAATATTTGTAGATGAGCTACACTTGGCACGCTACTTTTACGCGATCTCAGACATAAAGGT
GCGAGGAAGGTGCAAGTGTAATCTCCATGCCACTGTATGTGTGTATGACAACAGCAAATTGACATGCGAAT
GTGAGCACAACACTACAGGTCCAGACTGTGGGAAATGCAAGAAGAATTATCAGGGCCGACCTTGGAGTCCA
GGCTCCTATCTCCCCATCCCCAAAGGCACTGCAAATACCTGTATCCCCAGTATTTCCAGTATTGGTAATCC
TCCAAAGTTTAATAGGATATGGCCGAATATTTCTTCCCTTCAGGTTTCTAACCCAAAACAAGTTGCTCCCA
AATTAGCTTTGTCAACACTTTCTTCTGTTCAAGTTGCAAACCACAAGAGAGCGAATGTCTGCGACAACGAG
CTCCTGCACTGCCAGAACGGAGGGACGTGCCACAACAACGTGCGCTGCCTGTGCCCGGCCGCATACACGGG
CATCCTCTCCGAGAAGCTGCGGTGCGAGGAGGCTGGCAGCTGCGGCTCCGACTCTGGCCAGGGCGCGCCCC
CGCACGGCTCCCCAGCGCTGCTGCTGCAGACCACGCTGCTGGGAACCGCCAGCCCCCTGGTGTTCTAGGTG
TCAC
NOV14y,
13382329 SNP
for CG51051-07 SEQ ID NO: 210 480 aa SNP: Leu to Gln
Protein Sequence at position 468
MYLSRFLSIHALWVTVSSAAQPYPLVWGHYDLCKTQIYTEEGKAADYAACQPESTDMTKYLKVKLDPPDIT
CGDPPETFCAMGNPYMCNNECDASTPELAAPPELMFDFEGRHPSTFWQSATWKEYPKPLQVNITLSWSKTI
ELTDNIVITFESGRPDQMILEKSLDYGRTWQPYQYYATDCLDAFHMDPKSVKDLSQHTVLEIICTEEYSTG
YTTNSKIIHFEIKDRFAFFAGPRLRNMASLYGQLDTTKKLRDFFTAADLRIRLLRPAVGEIFVDELHLARY
FYAISDIKVRGRCKCNLHATVCVYDNSKLTCECEHNTTGPDCGKCKKNYQGRPWSPGSYLPIPKGTANTCI
PSISSIGNPPKFNRIWPNISSLEVSNPKQVAPAAALSTVSSVQVAAHKAANVCDNELLHCQNGGTCHNNVR
CLCPAAYTGILCEKLRCEEAGSCGSDSGQGAPPHGSPAALLQTTLLGTASPLVF

A ClustalW comparison of the above protein sequences yields the following sequence alignment shown in Table 14B.

TABLE 14B
Comparison of the NOV14 protein sequences.

| | | |
|---|---|---|
| NOV14a | -----MYLSRFLSIHALWVTVSSVMQPYPLVWGHYDLCKTQIYTEEGKVWDYMACQPEST | |
| NOV14b | -----MYLSRFLSIHALWVTVSSVMQPYPLVWGHYDLCKTQIYTEEGKVWDYMACQPEST | |
| NOV14c | ---------------------RSVMQPYPLVWGHYDLCKTQIYTEEGKVWDYMACQPEST | |
| NOV14d | ----------------------------------------------------RSCQPEST | |
| NOV14e | -----MYLSRFLSIHALWVTVSSVMQPYPLVWGHYDLCKTQIYTEEGKVWDYMACQPEST | |
| NOV14f | TRSPTMYLSRFLSIHALWVTVSSVMQPYPLVWGHYDLCKTQIYTEEGKVWDYMACQPEST | |
| NOV14g | ------TRSRQRS-RGRSISRGRHARTHPQTALLESSCENK-RADLVFIID--SSR--SV | |
| NOV14h | ------TRSRQRS-RGRSISRGRHARTHPQTALLESSCENK-RADLVFIID--SSR--SV | |
| NOV14i | ------TRSRQRS-RGRSISRGRHARTHPQTALLESSCENK-RADLVFIID--SSR--SV | |
| NOV14j | -----MYLSRFLSIHALWVTVSSVMQPYPLVWGHYDLCKTQIYTEEGKVWDYMACQPEST | |
| NOV14k | -----MYLSRFLSIHALWVTVSSVMQPYPLVWGHYDLCKTQIYTEEGKVWDYMACQPEST | |
| NOV14l | -----MYLSRFLSIHALWVTVSSVMQPYPLVWGHYDLCKTQIYTEEGKVWDYMACQPEST | |
| NOV14m | -----MYLSRFLSIHALWVTVSSVMQPYPLVWGHYDLCKTQIYTEEGKVWDYMACQPEST | |
| NOV14n | -----MYLSRFLSIHALWVTVSSVMQPYPLVWGHYDLCKTQIYTEEGKVWDYMACQPEST | |
| NOV14o | -----MYLSRFLSIHALWVTVSSVMQPYPLVWGHYDLCKTQIYTEEGKVWDYMACQPEST | |
| NOV14p | -----MYLSRFLSIHALWVTVSSVMQPYPLVWGHYDLCKTQIYTEEGKVWDYMACQPEST | |
| NOV14q | -----------------------VMQPYPLVWGHYDLCKTQIYTEEGKVWDYMACQPEST | |
| NOV14r | ----------------------------------------------GSAAAPFTCQPEST | |
| NOV14s | -----MYLSRFLSIHALWVTVSSVMQPYPLVWGHYDLCKTQIYTEEGKVWDYMACQPEST | |
| NOV14t | ------------------------------------------------------CQPEST | |
| NOV14u | -----MYLSRFLSIHALWVTVSSVMQPYPLVWGHYDLCKTQIYTEEGKVWDYMACQPEST | |
| NOV14v | -----MYLSRFLSIHALWVTVSSVMQPYPLVWGHYDLCKTQIYTEEGKVWDYMACQPEST | |
| NOV14a | DMTKYLKVKLDPPDITCG-DP-PETFCAMGNPYMCNNECDASTPELAHPPELMFDFE-GR | |
| NOV14b | DMTKYLKVKLDPPDITCG-DP-PETFCAMGNPYMCNNECDASTPELAHPPELMFDFE-GR | |
| NOV14c | DMTKYLKVKLDPPDITCG-DP-PETFCAMGNPYMCNNECDASTPELAHPPELMFDFE-GR | |
| NOV14d | DMTKYLKVKLDPPDITCG-DP-PETFCAMGNPYMCNNECDASTPELAHPPELMFDFE-GR | |
| NOV14e | DMTKYLKVKLDPPDITCG-DP-PETFCAMGNPYMCNNECDASTPELAHPPELMFDFE-GR | |
| NOV14f | DMTKYLKVKLDPPDITCG-DP-PETFCAMGNPYMCNNECDASTPELAHPPELMFDFE-GR | |
| NOV14g | NTHDYAKVKEFIVDILQFLDIGPDVTRVGLLQYGSTVKNEFSLKTFKRKSEVERAVKRMR | |
| NOV14h | NTHDYAKVKEFIVDILQFLDIGPDVTRVGLLQYGSTVKNEFSLKTFKRKSEVERAVKRMR | |
| NOV14i | NTHDYAKVKEFIVDILQFLDIGPDVTRVGLLQYGSTVKNEFSLKTFKRKSEVERAVKRMR | |
| NOV14j | DMTKYLKVKLDPPDITCG-DP-PETFCAMGNPYMCNNECDASTPELAHPPELMFDFE-GR | |
| NOV14k | DMTKYLKVKLDPPDITCG-DP-PETFCAMGNPYMCNNECDASTPELAHPPELMFDFE-GR | |
| NOV14l | DMTKYLKVKLDPPDITCG-DP-PETFCAMGNPYMCNNECDASTPELAHPPELMFDFE-GR | |
| NOV14m | DMTKYLKVKLDPPDITCG-DP-PETFCAMGNPYMCNNECDASTPELAHPPELMFDFE-GR | |
| NOV14n | DMTKYLKVKLDPPDITCG-DP-PETFCAMGNPYMCNNECDASTPELAHPPELMFDFE-GR | |
| NOV14o | DMTKYLKVKLDPPDITCG-DP-PETFCAMGNPYMCNNECDASTPELAHPPELMFDFE-GR | |
| NOV14p | DMTKYLKVKLDPPDITCG-DP-PETFCAMGNPYMCNNECDASTPELAHPPELMFDFE-GR | |
| NOV14q | DMTKYLKVKLDPPDITCG-DP-PETFCAMGNPYMCNNECDASTPELAHPPELMFDFE-GR | |
| NOV14r | DMTKYLKVKLDPPDITCG-DP-PETFCAMGNPYMCNNECDASTPELAHPPELMFDFE-GR | |
| NOV14s | DMTKYLKVKLDPPDITCG-DP-PETFCAMGNPYMCNNECDASTPELAHPPELMFDFE-GR | |
| NOV14t | DMTKYLKVKLDPPDITCG-DP-PETFCAMGNPYMCNNECDASTPELAHPPELMFDFE-GR | |
| NOV14u | DMTKYLKVKLDPPDITCG-DP-PETFCAMGNPYMCNNECDASTPELAHPPELMFDFE-GR | |
| NOV14v | DMTKYLKVKLDPPDITCG-DP-PETFCAMGNPYMCNNECDASTPELAHPPELMFDFE-GR | |
| NOV14a | HPSTFWQSATWKEYPKPLQVNITLSWSK-TIELTDNI--VITFES-GRP-D--QMILEKS | |
| NOV14b | HPSTFWQSATWKEYPKPLQVNITLSWSK-TIELTDNI--VITFES-GRP-D--QMILEKS | |
| NOV14c | HPSTFWQSATWKEYPKPLQVNITLSWSK-TIELTDNI--VITFES-GRP-D--QMILEKS | |
| NOV14d | HPSTFWQSATWKEYPKPLQVNITLSWSK-TIELTDNI--VITFES-GRP-D--QMILEKS | |
| NOV14e | HPSTFWQSATWKEYPKPLQVNITLSWSK-TIELTDNI--VITFES-GRP-D--QMILEKS | |
| NOV14f | HPSTFWQSATWKEYPKPLQVNITLSWSK-TIELTDNI--VITFES-GRP-D--QMILEKS | |
| NOV14g | HLSTG--TMTG--LAIQYALNIAFSEAEGARPLRENVPRVIMIVTDGRPQDSVAEVAAKA | |
| NOV14h | HLSTG--TMTG--LAIQYALNIAFSEAEGARPLRENVPRVIMIVTDGRPQDSVAEVAAKA | |
| NOV14i | HLSTG--TMTG--LAIQYALNIAFSEAEGARPLRENVPRVIMIVTDGRPQDSVAEVAAKA | |
| NOV14j | HPSTFWQSATWKEYPKPLQVNITLSWSK-TIELTDNI--VITFES-GRP-D--QMILEKS | |
| NOV14k | HPSTFWQSATWKEYPKPLQVNITLSWSK-TIELTDNI--VITFES-GRP-D--QMILEKS | |
| NOV14l | HPSTFWQSATWKEYPKPLQVNITLSWSK-TIELTDNI--VITFES-GRP-D--QMILEKS | |
| NOV14m | HPSTFWQSATWKEYPKPLQVNITLSWSK-TIELTDNI--VITFES-GRP-D--QMILEKS | |
| NOV14n | HPSTFWQSATWKEYPKPLQVNITLSWSK-TIELTDNI--VITFES-GRP-D--QMILEKS | |
| NOV14o | HPSTFWQSATWKEYPKPLQVNITLSWSK-TIELTDNI--VITFES-GRP-D--QMILEKS | |
| NOV14p | HPSTFWQSATWKEYPKPLQVNITLSWSK-TIELTDNI--VITFES-GRP-D--QMILEKS | |
| NOV14q | HPSTFWQSATWKEYPKPLQVNITLSWSK-TIELTDNI--VITFES-GRP-D--QMILEKS | |
| NOV14r | HPSTFWQSATWKEYPKPLQVNITLSWSK-TIELTDNI--VITFES-GRP-D--QMILEKS | |
| NOV14s | HPSTFWQSATWKEYPKPLQVNITLSWSK-TIELTDNI--VITFES-GRP-D--QMILEKS | |
| NOV14t | HPSTFWQSATWKEYPKPLQVNITLSWSK-TIELTDNI--VITFES-GRP-D--QMILEKS | |
| NOV14u | HPSTFWQSATWKEYPKPLQVNITLSWSK-TIELTDNI--VITFES-GRP-D--QMILEKS | |
| NOV14v | HPSTFWQSATWKEYPKPLQVNITLSWSK-TIELTDNI--VITFES-GRP-D--QMILEKS | |
| NOV14a | LDYGRTWQPYQYYATDC--LDAF----HMDPKS-VKDLSQHTVLEIICTEEYSTGYTTNS | |
| NOV14b | LDYGRTWQPYQYYATDC--LDAF----HMDPKS-VKDLSQHTVLEIICTEEYSTGYTTNS | |
| NOV14c | LDYGRTWQPYQYYATDC--LDAF----HMDPKS-VKDLSQHTVLEIICTEEYSTGYTTNS | |
| NOV14d | LDYGRTWQPYQYYATDC--LDAF----HMDPKS-VKDLSQHTVLEIICTEEYSTGYTTNS | |
| NOV14e | LDYGRTWQPYQYYATDC--LDAF----HMDPKS-VKDLSQHTVLEIICTEEYSTGYTTNS | |
| NOV14f | LDYGRTWQPYQYYATDC--LDAF----HMDPKS-VKDLSQHTVLEIICTEEYSTGYTTNS | |
| NOV14g | RDTGILIFAIGVGQVDFNTLKSIGSEPHEDHVFLVANFSQIETLTSVFQKKLCTAH-MCS | |
| NOV14h | RDTGILIFAIGVGQVDFNTLKSIGSEPHEDHVFLVANFSQIETLTSVFQKKLCTAH-MCS | |
| NOV14i | RDTGILIFAIGVGQVDFNTLKSIGSEPHEDHVFLVANFSQIETLTSVFQKKLCTAH-MCS | |
| NOV14j | LDYGRTWQPYQYYATDC--LDAF----HMDPKS-VKDLSQHTVLEIICTEEYSTGYTTNS | |
| NOV14k | LDYGRTWQPYQYYATDC--LDAF----HMDPKS-VKDLSQHTVLEIICTEEYSTGYTTNS | |
| NOV14l | LDYGRTWQPYQYYATDC--LDAF----HMDPKS-VKDLSQHTVLEIICTEEYSTGYTTNS | |
| NOV14m | LDYGRTWQPYQYYATDC--LDAF----HMDPKS-VKDLSQHTVLEIICTEEYSTGYTTNS | |
| NOV14n | LDYGRTWQPYQYYATDC--LDAF----HMDPKS-VKDLSQHTVLEIICTEEYSTGYTTNS | |
| NOV14o | LDYGRTWQPYQYYATDC--LDAF----HMDPKS-VKDLSQHTVLEIICTEEYSTGYTTNS | |
| NOV14p | LDYGRTWQPYQYYATDC--LDAF----HMDPKS-VKDLSQHTVLEIICTEEYSTGYTTNS | |
| NOV14q | LDYGRTWQPYQYYATDC--LDAF----HMDPKS-VKDLSQHTVLEIICTEEYSTGYTTNS | |
| NOV14r | LDYGRTWQPYQYYATDC--LDAF----HMDPKS-VKDLSQHTVLEIICTEEYSTGYTTNS | |
| NOV14s | LDYGRTWQPYQYYATDC--LDAF----HMDPKS-VKDLSQHTVLEIICTEEYSTGYTTNS | |
| N0V14t | LDYGRTWQPYQYYATDC--LDAF----HMDPKS-VKDLSQHTVLEIICTEEYSTGYTTNS | |
| NOV14u | LDYGRTWQPYQYYATDC--LDAF----HMDPKS-VKDLSQHTVLEIICTEEYSTGYTTNS | |
| NOV14v | LDYGRTWQPYQYYATDC--LDAF----HMDPKS-VKDLSQHTVLEIICTEEYSTGYTTNS | |
| NOV14a | KIIHFEIKDRFAFFAGPRLPNMASLYGQLD-TTKKLRDFFTVTDLRIRLLRPAVGEIFVD | |
| NOV14b | KIIHFEIKDRFAFFAGPRLRNMASLYGQLD-TTKKLRDFFTVTDLRIRLLRPAVGEIFVD | |
| NOV14c | KIIHFEIKDRFAFFAGPRLRNMASLYGQLD-TTKKLRDFFTVTDLRIRLLRPAVGEIFVD | |
| NOV14d | KIIHFEIKDRFAFFAGPRLRNMASLYGQLD-TTKKLRDFFTVTDLRIRLLRPAVGEIFVD | |
| NOV14e | KIIHFEIKDRFAFFAGPRLRNMASLYGQLD-TTKKLRDFFTVTDLRIRLLRPAVGEIFVD | |
| NOV14f | KIIHFEIKDRFAFFAGPRLRNMASLYGQLD-TTKKLRDFFTVTDLRIRLLRPAVGEIFVD | |
| NOV14g | TLEHNCAHFCINIPGSYVCRCKQGYILNSDQTTCRIQDLCAMEDHNCEQLCVNVPGSFVC | |
| NOV14h | TLEHNCAHFCINIPGSYVCRCKQGYILNSDQTTCRIQDLCANEDGBCEQKCVBVOGSFVC | |
| NOV14i | TLEHNCAHFCINIPGSYVCRCKQGYILNSDQTTCRIQDLCAMEDHNCEQLCVNVPGSFVC | |
| NOV14j | KIIHFEIKDRFAFFAGPRLRNMASLYGQLD-TTKKLRDFFTVTDLRIRLLRPAVGEIFVD | |
| NOV14k | KIIHFEIKDRFAFFAGPRLRNMASLYGQLD-TTKKLRDFFTVTDLRIRLLRPAVGEIFVD | |
| NOV14l | KIIHFEIKDRFAFFAGPRLRNMASLYGQLD-TTKKLRDFFTVTDLRIRLLRPAVGEIFVD | |
| NOVl4m | KIIHFEIKDRFAFFAGPRLRNMASLYGQLD-TTKKLRDFFTVTDLRIRLLRPAVGEIFVD | |
| NOV14n | KIIHFEIKDRFAFFAGPRLRNMASLYGQLD-TTKKLRDFFTVTDLRIRLLRPAVGEIFVD | |
| NOV14o | KIIHFEIKDRFAFFAGPRLRNMASLYGQLD-TTKKLRDFFTVTDLRIRLLRPAVGEIFVD | |
| NOV14p | KIIHFEIKDRFAFFAGPRLRNMASLYGQLD-TTKKLRDFFTVTDLRIRLLRPAVGEIFVD | |
| NOV14q | KIIHFEIKDRFAFFAGPRLRNMASLYGQLD-TTKKLRDFFTVTDLRIRLLRPAVGEIFVD | |
| NOV14r | KIIHFEIKDRFAFFAGPRLRNMASLYGQLD-TTKKLRDFFTVTDLRIRLLRPAVGEIFVD | |
| NOV14s | KIIHFEIKDRFAFFAGPRLRNMASLYGQLD-TTKKLRDFFTVTDLRIRLLRPAVGEIFVD | |
| NOV14t | KIIHFEIKDRFAFFAGPRLRNMASLYGQLD-TTKKLRDFFTVTDLRIRLLRPAVGEIFVD | |
| NOV14u | KIIHFEIKDRFAFFAGPRLRNMASLYGQLD-TTKKLRDFFTVTDLRIRLLRPAVGEIFVD | |
| NOV14v | KIIHFEIKDRFAFFAGPRLRNMASLYGQLD-TTKKLRDFFTVTDLRIRLLRPAVGEIFVD | |
| NOV14a | ELHLARYFYAISDIK---VRGRCKCNLH--ATVCVYDNSKLTCEC-EHNTTGPD---CGK | |
| NOV14b | ELHLARYFYAISDIK---VRGRCKCNLH--ATVCVYDNSKLTCEC-EHNTTGPD---CGK | |
| NOV14c | ELHLARYFYAISDIK---VRGRCKCNLH--ATVCVYDNSKLTCEC-EHNTTGPD---CGK | |
| NOV14d | ELHLARYFYAISDIK---VRGLE------------------------------------- | |
| NOV14e | ELHLARYFYAISDIK---VRGRCKCNLH--ATVCVYDNSKLTCEC-EHNTTGPD---CGK | |
| NOV14f | ELHLARYFYAISDIK---VRGRCKCNLH--ATVCVYDNSKLTCEC-EHNTTGPD---CGK | |
| NOV14g | QC-YSGYALAE-DGKRCVAVDYCASENHGCEHECVNADGSYLCQCHEGFALNPDEKTCTK | |
| NOV14h | QC-YSGYALAE-DGKRCVAVDYCASENHGCEHECVNADGSYLCQCHEGFALNPDEKTCTK | |
| NOV14i | QC-YSGYALAE-DGKRCVAVDYCASENHGCEHECVNADGSYLCQCHEGFALNPDEKTCTK | |
| NOV14j | ELHLARYFYAISDIK---VRGRCKCNLH--ATVCVYDNSKLTCEC-EHNTTGPD---CGK | |
| NOV14k | ELHLARYFYAISDIK---VRGRCKCNLH--ATVCVYDNSKLTCEC-EHNTTGPD---CGK | |
| NOV14l | ELHLARYFYAISDIK---VRGRCKCNLH--ATVCVYDNSKLTCEC-EHNTTGPD---CGK | |
| NOV14m | ELHLARYFYAISDIK---VRGRCKCNLH--ATVCVYDNSKLTCEC-EHNTTGPD---CGK | |
| NOV14n | ELHLARYFYAISDIK---VRGRCKCNLH--ATVCVYDNSKLTCEC-EHNTTGPD---CGK | |
| NOV14o | ELHLARYFYAISDIK---VRGRCKCNLH--ATVCVYDNSKLTCEC-EHNTTGPD---CGK | |
| NOV14p | ELHLARYFYAISDIK---VRGRCKCNLH--ATVCVYDNSKLTCEC-EHNTTGPD---CGK | |
| NOV14q | ELHLARYFYAISDIK---VRGRCKCNLH--ATVCVYDNSKLTCEC-EHNTTGPD---CGK | |
| NOV14r | ELHLARYFYAISDIK---VRGKGGRA---------------------------------- | |
| NOV14s | ELHLARYFYAISDIK---VRGRCKCNLH--ATVCVYDNSKLTCEC-EHNTTGPD---CGK | |
| NOV14t | ELHLARYFYAISDIK---VRG--------------------------------------- | |
| NOV14u | ELHLARYFYAISDIK---VRGRCKCNLH--ATVCVYDNSKLTCEC-EHNTTGPD---CGK | |
| NOV14v | ELHLARYFYAISDIK---VRGRCKCNLH--ATVCVYDNSKLTCEC-EHNTTGPD---CGK | |
| NOV14a | ---CKKNYQG-RPW---SPGSY-LPIPKG-TANT----C--I--PSISSIG--------- | |
| NOV14b | ---CKKNYQG-RPW---SPGSY-LPIPKG-TANT----C--I--PSISSIGNPPKFNRIW | |
| NOV14c | ---CKKNYQG-RPW---SPGSY-LPIPKG-TANT----C--I--PSISSIGNPPKFNRIW | |
| NOV14d | ------------------------------------------------------------ | |
| NOV14e | ---CKKNYQG-RPW---SPGSY-LPIPKG-TANT----C--I--PSISSIGNPPKFNRIW | |
| NOV14f | ---CKKNYQG-RPW---SPGSY-LPIPKG-TANT----C--I--PSISSIGNPPKFNRIW | |
| NOV14g | IDYCASSNHGCQHECVNTDDSYSCHCLKGFTLNPDKKTCRRINYCALNKPGCEHECVNME | |
| NOV14h | IDYCASSNHGCQHECVNTDDSYSCHCLKGFTLNPDKKTCRRINYCALNKPGCEHECVNME | |
| NOV14i | IDYCASSNHGCQHECVNTDDSYSCHCLKGFTLNPDKKTCRRINYCALNKPGCEHECVNME | |
| NOV14j | ---CKKNYQG-RPW---SPGSY-LPIPKG-TANT----C--I--PSISSIGNPPKFNRIW | |
| NOV14k | ---CKKNYQG-RPW---SPGSY-LPIPKG-TANT----C--I--PSISSIG--------- | |
| NOV14l | ---CKKNYQG-RPW---SPGSY-LPIPKG-TAN--------------------------- | |
| NOV14m | ---CKKNYQG-RPW---SPGSY-LPIPKG-TANT----C--I--PSISSIG--------- | |
| NOV14n | ---CKKNYQG-RPW---SPGSY-LPIPKG-TANT----C--I--PSISSIG--------- | |
| NOV14o | ---CKKNYQG-RPW---SPGSY-LPIPKG-TANT----C--I--PSISSIG--------- | |
| NOV14p | ---CKKNYQG-RPW---SPGSY-LPIPKG-TANT----C--I--PSISSIGNPPKFNRIW | |
| NOV14q | ---CKKNYQG-RPW---SPGSY-LPIPKG-TANT----C--I--PSISSIGNPPKFNRIW | |
| NOV14r | ------------------------------------------------------------ | |
| NOV14s | ---CKKNYQG-RPW---SPGSY-LPIPKG-TANT----C--I--PSISSIGNPPKFNRIW | |
| NOV14t | ------------------------------------------------------------ | |
| NOV14u | ---CKKNYQG-RPW---SPGSY-LPIPKG-TANT----C--I--PSISSIGNPPKFNRIW | |
| NOV14v | ---CKKNYQG-RPW---SPGSY-LPIPKG-TANT----C--I--PSISSIGNPPKFNRIW | |
| NOV14a | PNISSLEVSNPKQVAPKLALSTVSSVQVANHKRANVCDN--ELLHCQNGG---------T | |
| NOV14b | PNISSLEVSNPKQVAPKLALSTVSSVQVANHKRANVCDN--ELLHCQNGG---------T | |
| NOV14c | PNISSLEVSNPKQVAPKLALSTVSSVQVANHKRANVCDN--ELLHCQNGG---------T | |
| NOV14d | ------------------------------------------------------------ | |
| NOV14e | PNISSLEVSNPKQVAPKLALSTVSSVQVANHKRANVCDN--ELLHCQNGG---------T | |
| NOV14f | PNISSLEVSNPKQVAPKLALSTVSSVQVANHKRANVCDN--ELLHCQNGG---------T | |
| NOV14g | ESYYCRCHRGYTLDPNOKTCSRVDHCAQQDHGCEQLCLNTEDSFVCQCSEGFLINEDLKT | |
| NOV14h | ESYYCRCHRCYTLDPNCKTCSRVDHCAQQDHGCEQLCLNTEDSFVCQCSEGFLINEDLKT | |
| NOV14i | ESYYCRCHRGYTLDPNGKTCSRVDHCAQQDHGCEQLCLNTEDSFVCQCSEGFLINEDLKT | |
| NOV14j | PNISSLEVSNPKQVAPKLALSTVSSVQVANHKRANVCDN--ELLHCQNGG---------T | |
| NOV14k | ----------------------------------KCYCN--PLGSIHD-----------R | |
| NOV14l | --------------------------------TSNVCDN--ELLHCQNGG---------T | |
| NOV14m | ---------------------------------TNVCDN--ELLHCQNGG---------T | |
| NOV14n | ---------------------------------TNVCDN--ELLHCQNGG---------T | |
| NOV14o | ---------------------------------TNVCDN--ELLHCQNGG---------T | |
| NOV14p | PNISSLEVSNPK--------------------QANVCDN--ELLHCQNGG---------T | |
| NOV14q | PNISSLEVSNPKQVAPKLALSTVSSVQVANHKRANVCDN--ELLHCQNGG---------T | |
| NOV14r | ------------------------------------------------------------ | |
| NOV14s | PNISSLEVSNPKQVAPKLALSTVSSVQVANHKRANVCDN--ELLHCQNGG---------T | |
| NOV14t | ------------------------------------------------------------ | |
| NOV14u | PNISSLEVSNPKQVAPKLALSTVSSVQVANHKRANVCDN--ELLHCQNGG---------T | |
| NOV14v | PNISSLEVSNPKQVAPKLALSTVSSVQVANHKRANVCDN--ELLHCQNGG---------T | |
| NOV14a | CHNNVRCLCPAAYTGILCE------KLRCEEAGSCGSD-------SGQGAPPHGSPALLL | |
| NOV14b | CHNNVRCLCPAAYTGILCE------KLRCEEAGSCGSD-------SGQGAPPHGSPALLL | |
| NOV14c | CHNNVRCLCPAAYTGILCE------KLRCEEAGSCGSD-------SGQGAPPHGSPALLL | |
| NOV14d | ------------------------------------------------------------ | |
| NOV14e | CHNNVRCLCPAAYTGILCE------KLRCEEAGSCGSD-------SGQGAPPHGSPALLL | |
| NOV14f | CHNNVRCLCPAAYTGILCE------KLRCEEAGSCGSD-------SGQGAPPHGSPALLL | |
| NOV14g | CSRVDYCLLSDHGCEYSCVNMDRSFACQCPEGHVLRSDGKTCAKLDSCALGDHGCEHSCV | |
| NOV14h | CSRVDYCLLSDHGCEYSCVNMDRSFACQCPEGHVLRSDGKTCAKLDSCALGDHGCEHSCV | |
| NOV14i | CSRVDYCLLSDHGCEYSCVNMDRSFACQCPEGHVLRSDGKTCAKLDSCALGDHGCEHSCV | |
| NOV14j | CHNNVRCLCPAAYTGILCE------KLRCEEAGSCGSD-------SGQGAPPHGSPALLL | |
| NOV14k | CNGSGFCECKTGTTGPKCD------ECLPGNSWHYGCQP---NVCDNELLPPCICQDSCR | |
| NOV14l | CHNNVRCLCPAAYTGILCE------KLRCEEAGSCGSD-------SGQGAPPHGSPALLL | |
| NOV14m | CHNNVRCLCPAAYTGILCE------KLRCEEAGSCGSD-------SGQGAPPHGSPALLL | |
| NOV14n | CHNNVRCLCPAAYTGILCE------KLRCEEAGSCGSD-------SGQGAPPHGSPALLL | |
| NOV14o | CHNNVRCLCPAAYTGILCE------KLRCEEAGSCGSD-------SGQGAPPHGSPALLL | |
| NOV14p | CHNNVRCLCPAAYTGILCE------KLRCEEAGSCGSD-------SGQGAPPHGSPALLL | |
| NOV14q | CHNNVRCLCPAAYTGILCE------KLRCEEAGSCGSD-------SGQGAPPHGSPALLL | |
| NOV14r | ------------------------------------------------------------ | |
| NOV14s | CHNNVRCLCPAAYTGILCE------KLRCEEAGSCGSD-------SGQGAPPHGSPALLL | |
| NOV14t | ------------------------------------------------------------ | |
| NOV14u | CHNNVRCLCPAAYTGILCE------KLRCEEAGSCGSD-------SGQGAPPHGSPALLL | |
| NOV14v | CHNNVRCLCPAAYTGILCE------KLRCEEAGSCGSD-------SGQGAPPHGSPALLL | |
| NOV14a | L---TTLLGTASPLVF-------------------------------------------- | |
| NOV14b | L---TTLLGTASPLVF-------------------------------------------- | |
| NOV14d | ------------------------------------------------------------ | |
| NOV14e | L---TTLLGTASPLVF-------------------------------------------- | |
| NOV14f | L---TTLLGTASPLVFLEG----------------------------------------- | |
| NOV14g | SSEDSFVCQCFEGYILREDGKTCRRKDVCQAIDHGCEHICVNSDDSYTCECLEGFRLAED | |
| NOV14h | SSEDSFVCQCFEGYILREDGKTCRRKDVCQAIDHGCEHICVNSDDSYTCECLEGFRLAED | |
| NOV14i | SSEDSFVCQCFEGYILREDGKTCRRKDVCQAIDHGCEHICVNSDDSYTCECLEGFRLAED | |
| NOV14j | L---TTLLGTASPLVF-------------------------------------------- | |
| NOV14k | F---YLWPGRAPARLPREG----------------------------------------- | |
| NOV14l | L---TTLLGTASPLVF-------------------------------------------- | |
| NOV14m | L---TTLLGTASPLVF-------------------------------------------- | |
| NOV14n | L---TTLLGTASPLVF-------------------------------------------- | |
| NOV14o | S---TTLD---------------------------------------------------- | |
| NOV14p | L---TTLLGTASPLVF-------------------------------------------- | |
| NOV14q | L---TTLLGTASPLVF-------------------------------------------- | |
| NOV14r | ------------------------------------------------------------ | |
| NOV14s | L---TTLLGTASPLVF-------------------------------------------- | |
| NOV14t | ------------------------------------------------------------ | |
| NOV14u | L---TTLLGTASPLVF-------------------------------------------- | |
| NOV14v | L---TTLLGTASPLVF | ------------------------------------------- |
| NOV14a | ------------------------------------------------------------ | |
| NOV14b | ------------------------------------------------------------ | |
| NOV14c | ------------------------------------------------------------ | |
| NOV14d | ------------------------------------------------------------ | |
| NOV14e | ------------------------------------------------------------ | |
| NOV14f | ------------------------------------------------------------ | |
| NOV14g | GKRCRRKDVCKSTHHGCEHICVNNGNSYICKCSEGFVLAEDGRRCKKCTEGPIDLVFVID | |
| NOV14h | GKRCRRKDVCKSTHHGCEHICVNNGNSYICKCSEGFVLAEDGRRCKKCTEGPIDLVFVID | |
| NOV14i | GKRCRRKDVCKSTHHGCEHICVNNGNSYICKCSEGFVLAEDGRRCKKCTEGPIDLVFVID | |
| NOV14j | ------------------------------------------------------------ | |
| NOV14k | ------------------------------------------------------------ | |
| NOV14l | ------------------------------------------------------------ | |
| NOV14m | ------------------------------------------------------------ | |
| NOV14n | ------------------------------------------------------------ | |
| NOV14o | ------------------------------------------------------------ | |
| NOV14p | ------------------------------------------------------------ | |
| NOV14q | ------------------------------------------------------------ | |
| NOV14r | ------------------------------------------------------------ | |
| NOV14s | ------------------------------------------------------------ | |
| NOV14t | ------------------------------------------------------------ | |
| NOV14u | ------------------------------------------------------------ | |
| NOV14v | ------------------------------------------------------------ | |
| NOV14a | ------------------------------------------------------------ | |
| NOV14b | ------------------------------------------------------------ | |
| NOV14c | ------------------------------------------------------------ | |
| NOV14d | ------------------------------------------------------------ | |
| NOV14e | ------------------------------------------------------------ | |
| NOV14f | ------------------------------------------------------------ | |
| NOV14g | GSKSLGEENFEVVKQFVTGIIDSLTISPKAARVGLLQYSTQVHTEFTLRNFNSAKDMKKA | |
| NOV14h | ------------------------------------------------------------ | |
| NOV14i | GSKSLGEENFEVVKQFVTGIIDSLTISPKAARVGLLQYSTQVHTEFTLRNFNSAKDMKKA | |
| NOV14j | ------------------------------------------------------------ | |
| NOV14k | ------------------------------------------------------------ | |
| NOV14l | ------------------------------------------------------------ | |
| NOV14m | ------------------------------------------------------------ | |
| NOV14n | ------------------------------------------------------------ | |
| NOV14o | ------------------------------------------------------------ | |
| NOV14p | ------------------------------------------------------------ | |
| NOV14q | ------------------------------------------------------------ | |
| NOV14r | ------------------------------------------------------------ | |
| NOV14s | ------------------------------------------------------------ | |
| NOV14t | ------------------------------------------------------------ | |
| NOV14u | ------------------------------------------------------------ | |
| NOV14v | ------------------------------------------------------------ | |
| NOV14a | ------------------------------------------------------------ | |
| NOV14b | ------------------------------------------------------------ | |
| NOV14c | ------------------------------------------------------------ | |
| NOV14d | ------------------------------------------------------------ | |
| NOV14e | ------------------------------------------------------------ | |
| NOV14f | ------------------------------------------------------------ | |
| NOV14g | VAHMKYMGKGSMTGLALKHMFERSFTQGEGARPLSTRVPRAAIVFTDGRAQDDVSEWASK | |
| NOV14h | ------------------------------------------------------------ | |
| NOV14i | VAHMKYMGKGSMTGLALKHMFERSFTQGEGARPLSTRVPRAAIVFTDGRAQDDVSEWASK | |
| NOV14j | ------------------------------------------------------------ | |
| NOV14k | ------------------------------------------------------------ | |
| NOV14l | ------------------------------------------------------------ | |
| NOV14m | ------------------------------------------------------------ | |
| NOV14n | ------------------------------------------------------------ | |
| NOV14o | ------------------------------------------------------------ | |
| NOV14p | ------------------------------------------------------------ | |
| NOV14q | ------------------------------------------------------------ | |
| NOV14r | ------------------------------------------------------------ | |
| NOV14s | ------------------------------------------------------------ | |
| NOV14t | ------------------------------------------------------------ | |
| NOV14u | ------------------------------------------------------------ | |
| NOV14v | ------------------------------------------------------------ | |
| NOV14a | ------------------------------------------------------------ | |
| NOV14b | ------------------------------------------------------------ | |
| NOV14c | ------------------------------------------------------------ | |
| NOV14d | ------------------------------------------------------------ | |
| NOV14e | ------------------------------------------------------------ | |
| NOV14f | ------------------------------------------------------------ | |
| NOV14g | AKANGITMYAVGVGKAIEEELQEIASEPTNKHLFYAEDFSTMDEISEKLKKGICEALEDS | |
| NOV14h | -RCKSITMYAVGVGKAIEEELQEIASEPTNKHLFYAEDFSTMDEISEKLKKGICEALEDS | |
| NOV14i | AKANGITMYAVGVGKAIEEELQEIASEPTNKHLFYAEDFSTMDEISEKLKKGICEALEDS | |
| NOV14j | ------------------------------------------------------------ | |
| NOV14k | ------------------------------------------------------------ | |
| NOV14l | ------------------------------------------------------------ | |
| NOV14m | ------------------------------------------------------------ | |
| NOV14n | ------------------------------------------------------------ | |
| NOV14o | ------------------------------------------------------------ | |
| NOV14p | ------------------------------------------------------------ | |
| NOV14q | ------------------------------------------------------------ | |
| NOV14r | ------------------------------------------------------------ | |
| NOV14s | ------------------------------------------------------------ | |
| NOV14t | ------------------------------------------------------------ | |
| NOV14u | ------------------------------------------------------------ | |
| NOV14v | ------------------------------------------------------------ | |
| NOV14a | ------------------------------------------------------------ | |
| NOV14b | ------------------------------------------------------------ | |
| NOV14c | ------------------------------------------------------------ | |
| NOV14d | ------------------------------------------------------------ | |
| NOV14e | ------------------------------------------------------------ | |
| NOV14f | ------------------------------------------------------------ | |
| NOV14g | DGRQDSPAGELPKTVQQPTESEPVTINIQDLLSCSNFAVQHRYLFEEDNLLRSTQKLSHS | |
| NOV14h | ------------------------------------------------------------ | |
| NOV14i | DGRQDSPAGELPKTVQQPTESEPVTINIQDLLSCSNFAVQHRYLFEEDNLLRSTQKLSHS | |
| NOV14j | ------------------------------------------------------------ | |
| NOV14k | ------------------------------------------------------------ | |
| NOV14l | ------------------------------------------------------------ | |
| NOV14m | ------------------------------------------------------------ | |
| NOV14n | ------------------------------------------------------------ | |
| NOV14o | ------------------------------------------------------------ | |
| NOV14p | ------------------------------------------------------------ | |
| NOV14q | ------------------------------------------------------------ | |
| NOV14r | ------------------------------------------------------------ | |
| NOV14s | ------------------------------------------------------------ | |
| NOV14t | ------------------------------------------------------------ | |
| NOV14u | ------------------------------------------------------------ | |
| NOV14v | ------------------------------------------------------------ | |
| NOV14a | ---------------------------------------------------------- | |
| NOV14b | ---------------------------------------------------------- | |
| NOV14c | ---------------------------------------------------------- | |
| NOV14d | ---------------------------------------------------------- | |
| NOV14e | ---------------------------------------------------------- | |
| NOV14f | ---------------------------------------------------------- | |
| NOV14g | TKPSGSPLEEKHDQCKCENLIMFQNLANEEVRKLTQRLEEMTQRMEALENRLRYRVDG | |
| NOV14h | TKPSGSPLEEKHDQCKCENLIMFQNLANEEVRKLTQRLEEMTQRMEALENRLRYRVDG | |
| NOV14i | TKPSGSPLEEKHDQCKCENLIMFQNLANEEVRKLTQRLEEMTQRMEALENRLRYRVDG | |
| NOV14j | ---------------------------------------------------------- | |
| NOV14k | ---------------------------------------------------------- | |
| NOV14l | ---------------------------------------------------------- | |
| NOV14m | ---------------------------------------------------------- | |
| NOV14n | ---------------------------------------------------------- | |
| NOV14o | ---------------------------------------------------------- | |
| NOV14p | ---------------------------------------------------------- | |
| NOV14q | ---------------------------------------------------------- | |
| NOV14r | ---------------------------------------------------------- | |
| NOV14s | ---------------------------------------------------------- | |
| NOV14t | ---------------------------------------------------------- | |
| NOV14u | ---------------------------------------------------------- | |
| NOV14v | ---------------------------------------------------------- | |
| NOV14a | (SEQ ID NO: 162) | |
| NOV14b | (SEQ ID NO: 164) | |
| NOV14c | (SEQ ID NO: 166) | |
| NOV14d | (SEQ ID NO: 168) | |
| NOV14e | (SEQ ID NO: 170) | |
| NOV14f | (SEQ ID NO: 172) | |
| NOV14g | (SEQ ID NO: 174) | |
| NOV14h | (SEQ ID NO: 176) | |
| NOV14i | (SEQ ID NO: 178) | |
| NOV14j | (SEQ ID NO: 180) | |
| NOV14k | (SEQ ID NO: 182) | |
| NOV14l | (SEQ ID NO: 184) | |
| NOV14m | (SEQ ID NO: 186) | |
| NOV14n | (SEQ ID NO: 188) | |
| NOV14o | (SEQ ID NO: 190) | |
| NOV14p | (SEQ ID NO: 192) | |
| NOV14q | (SEQ ID NO: 194) | |
| NOV14r | (SEQ ID NO: 196) | |
| NOV14s | (SEQ ID NO: 198) | |
| NOV14t | (SEQ ID NO: 200) | |
| NOV14u | (SEQ ID NO: 204) | |
| NOV14v | (SEQ ID NO: 206) | |

Further analysis of the NOV14a protein yielded the following properties shown in Table 14C.

TABLE 17C
Protein Sequence Properties NOV14a

| SignalP | Cleavage site between residues 19 and 20 | | | | |
|---|---|---|---|---|---|
| analysis: | | | | | |
| PSORT II | PSG: a new signal peptide prediction method | | | | |
| analysis: | N-region: length 5; pos. chg 1; neg. chg 0 | | | | |
| | H-region: length 25; peak value 8.10 | | | | |
| | PSG score: 3.70 | | | | |
| | GvH: von Heijne's method for signal seq. recognition | | | | |
| | GvH score (threshold: −2.1): −5.81 | | | | |
| | possible cleavage site: between 18 and 19 | | | | |
| | >>> Seems to have no N-terminal signal peptide | | | | |
| | ALOM: Klein et al's method for TM region allocation | | | | |
| | Init position for calculation: 1 | | | | |
| | Tentative number of TMS(s) for the threshold 0.5: 1 | | | | |
| | Number of TMS(s) for threshold 0.5: 1 | | | | |
| | INTEGRAL | Likelihood = | −2.87 | Transmembrane | 464-480 |
| | PERIPHERAL | Likelihood = | 3.34 (at 3) | | |

| ALOM score: −2.87 (number of TMSs: 1) | | | |
|---|---|---|---|
| MTOP: Prediction of membrane topology (Hartmann et al.) | | | |
| Center position for calculation: 471 | | | |
| Charge difference: 0.5 C(0.0) - N(−0.5) | | | |
| C > N: C-terminal side will be inside | | | |
| >>>Caution: Inconsistent mtop result with signal peptide | | | |
| >>>Single TMS is located near the C-terminus | | | |
| >>>membrane topology: type Nt (cytoplasmic tail 1 to 463) | | | |
| MITDISC: discrimination of mitochondrial targeting seq | | | |
| R content: | 1 | Hyd Moment (75): | 7.91 |
| Hyd Moment (95): | 7.87 | G content: | 1 |
| D/E content: | 1 | S/T content: | 5 |

```
Score: −2.60
Gavel: prediction of cleavage sites for mitochondrial preseq
R-2 motif at 15 SRF|LS
NUCDISC: discrimination of nuclear localization signals
pat4: none
pat7: none
bipartite: none
content of basic residues: 9.6%
NLS Score: −0.47
KDEL: ER retention motif in the C-terminus: none
ER Membrane Retention Signals:
XXRR-like motif in the N-terminus: YLSR
none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: too long tail
Dileucine motif in the tail: found
LL at 266
LL at 412
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: nuclear
Reliability: 70.6
COIL: Lupas's algorithm to detect coiled-coil regions
total: 0 residues
Final Results (k = 9/23):
30.4%: nuclear
21.7%: cytoplasmic
13.0%: Golgi
13.0%: mitochondrial
  8.7%: endoplasmic reticulum
  4.3%: peroxisomal
  4.3%: plasma membrane
  4.3%: vesicles of secretory system
>> prediction for CG51051-07 is nuc (k = 23)
```

A search of the NOV14a protein against the Geneseq database, a proprietary database that contains sequences published in patents and patent publication, yielded several homologous proteins shown in Table 14D.

TABLE 14D
Geneseq Results for NOV14a

| | | NOV14a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAB49651 | Human SEC3 protein sequence SEQ | 1 . . . 480 | 480/480 (100%) | 0.0 |
| | ID 6 - Homo sapiens, 480 aa. | 1 . . . 480 | 480/480 (100%) | |
| | [WO200070046-A2, 23 NOV. 2000] | | | |
| ABB05421 | Mouse membrane bound type netrin | 1 . . . 480 | 461/480 (96%) | 0.0 |
| | protein SEQ ID NO: 14 - Mus | 1 . . . 480 | 467/480 (97%) | |
| | musculus, 480 aa. [JP2001327289- | | | |
| | A, 27 NOV. 2001] | | | |
| AAB05422 | Mouse membrane bound type netrin | 1 . . . 480 | 441/480 (91%) | 0.0 |
| | protein SEQ ID NO: 16 - Mus | 1 . . . 480 | 447/480 (92%) | |
| | musculus, 460 aa. [JP2001327289- | | | |
| | A, 27 NOV. 2001] | | | |
| ABU07468 | Protein differentially regulated in | 1 . . . 363 | 362/363 (99%) | 0.0 |
| | prostate cancer #71 - Homo sapiens, | 1 . . . 363 | 363/363 (99%) | |
| | 364 aa. [WO200281638-A2, 17 | | | |
| | OCT. 2002] | | | |
| ABU07437 | Protein differentially regulated in | 1 . . . 363 | 362/363 (99%) | 0.0 |
| | prostate cancer #40 - Homo sapiens, | 1 . . . 363 | 363/363 (99%) | |
| | 364 aa. [WO200281638-A2, 17 | | | |
| | OCT. 2002] | | | |

In a BLAST search of public sequence databases, the NOV14a protein was found to have homology to the proteins shown in the BLASTP data in Table 14E.

TABLE 14E
Public BLASTP Results for NOV14a

| | | | Identities/ | |
|---|---|---|---|---|
| Protein | | NOV14a | Similarities for | |
| Accession | | Residues/ | the Matched | |
| Number | Protein/Organism/Length | Match Residues | Portion | Expect Value |
| CAC21786 | Sequence 5 from Patent | 1 . . . 480 | 480/480 (100%) | 0.0 |
| | WO0070046 - Homo sapiens | 1 . . . 480 | 480/480 (100%) | |
| | (Human), 480 aa. | | | |
| Q9ESR6 | Netrin-G1d - Mus musculus | 1 . . . 480 | 461/480 (96%) | 0.0 |
| | (Mouse), 480 aa. | 1 . . . 480 | 467/480 (97%) | |
| Q8R4F8 | Laminet-1D - Mus musculus | 1 . . . 480 | 460/480 (95%) | 0.0 |
| | domesticus (western European | 1 . . . 480 | 466/480 (96%) | |
| | house mouse), 480 aa. | | | |
| Q9ESR7 | Netrin-G1e (Netrin G1) - Mus | 1 . . . 480 | 441/480 (91%) | 0.0 |
| | musculus (Mouse), 460 aa. | 1 . . . 460 | 447/480 (92%) | |
| Q8R4F7 | Laminet-1E - Mus musculus | 1 . . . 480 | 440/480 (91%) | 0.0 |
| | domesticus (western European | 1 . . . 460 | 446/480 (92%) | |
| | house mouse), 460 aa. | | | |

PFam analysis predicts that the NOV14a protein contains the domains shown in the Table 14F.

TABLE 14F
Domain Analysis of NOV14a

| | | Identities/ | |
|---|---|---|---|
| Pfam | NOV14a Match | Similarities | Expect |
| Domain | Region | for the Matched Region | Value |
| laminin_Nterm | 50 . . . 295 | 73/286 (26%) | 1.7e−12 |
| | | 134/286 (47%) | |
| laminin_EGF | 297 . . . 341 | 19/59 (32%) | 1.5e−05 |
| | | 30/59 (51%) | |
| EGF | 408 . . . 438 | 15/47 (32%) | 0.55 |
| | | 21/47 (45%) | |

### Example 15

The NOV15 clone was analyzed, and the nucleotide and encoded polypeptide sequences are shown in Table 15A.

TABLE 15A
NOV15 Sequence Analysis

NOV15a,
CG52261-01 SEQ ID NO: 211 937 bp
DNA Sequence ORF Start: ATG at 317 ORF Stop: TAG at 644
TCAGTCCTGGTCCCTCCCCTTCTTGGGTTCCTCATCCTGCTCTTCTAAATGTCGAGATGCCTGCAGCA
GTTACGCTTATCTCTGGCCACTATCTCTGCTTTTATCTCCTTTCTTAAAAGTCTTCAATGTCTCTAGG
CTCGTGTGTAAAGTCCTCTATCTTCAGTTACTACACCCTTTTCACCTTCAAAATCCTATGCGCACCTC
AAACTCAGCAAGTGTTAACTGAATTAGTCATCTTTGCTGCCATCGGCTGCCAACCTCCACTGTGGCCT
ACTGTGTGTTTCAAAGATGGCTCCGGAAATTATTCCCGTCCCACATGCTCTTTTCCAACGTGACCCTG
CCATCCCCAATGACAGTGGGAGTCCAATCCTCTCCTCTTGAATCTGGGCTGGCTCTTAGGACTCTTGT
CACCAAAAGGATGTCGCAGAAGTGGCACTGTTCAACTTTTGAGGCTAGGCTGAAAAGACTGTACAGCT
TTCTCCTGGTTCTACTAGAAGGCTCCCCCCTACAGAAGCTCGCCTCTCTCAAACCCAGCAGCCGTGCC
AATGGCAGCCCAACGCACAGGAGAGGCTTGCATGTGCTTCAGTCACCAGCTCCAGATGAGCCCAGTTT
TCTGCTAACACTTCCCACCTGTCAGATGTGCTAGCGAGGGCACCTCCAGATGACTCCAGTCCTCACCC
AGCTGAGTCACCTGTCATTTGAATTCTTCCAGCTGAGGCTCCCAGACATTGTCAGACAGAGACAAGCC
ATCCACCATCTCTGTGCCCCGTCCAAACTCCTGACCCACGCAGTCCATAAGGAAGAGGTTCTATGCAA
CTAAGTTTGGGATGATGTGTTACACAGCAGTACCCACCACACCCAACAAAACCACCAGTGCTTCCTCG
CTCCCTCTGCCTAAGACATGTGTTTCTGCACATCCATTCACACAGCCAAGAAG
NOV15a,
CG52261-01
Protein Sequence SEQ ID NO: 212 109 aa MW at 12122.2 kD
MLFCNVTLPSPMTVGVQSSPLESGLALRTLVTKRMWQKWHCSTFEARLKRLYSFLLVLLECSPLQKLA
SLKPSSRANGSPTHRRGLHVLQSPAPDEPSFLVTLPTCQMC
NOV15b,
268667469 SEQ ID NO: 213 346 bp
DNA Sequence ORF Start: at 2 ORF Stop: end of sequence
CACCCGATCCATGCTCTTTTGCAACGTGACCCTGCCATCCCCAATGACAGTGGGAGTCCAATCCTCTC
CTCTTGAATCTGGGCTGGCTCTTAGGACTCTTGTCACCAAAAGGATGTGGCAGAAGTGGCACTGTTCA
ACTTTTCAGGCTAGGCTGAAAAGACTGTACAGCTTTCTCCTGGTTCTACTAGAAGGCTCCCCCCTACA
GAAGCTCGCCTCTCTCAAACCCAGCAGCCGTGCCAATGGCAGCCCAACGCACGGGAGAGGCTTGCATG
TGCTTCAGTCACCAGCTCCAGATGAGCCCAGTTTTCTGGTAACACTTCCCACCTGTCAGATGTGCCTC
NOV15b,
268667469
Protein Sequence SEQ ID NO: 214 115 aa MW at 12567.6 kD
TGSMLFCNVTLPSPMTVGVQSSPLESGLALRTLVTKRMWQKWHCSTFEARLKRLYSFLLVLLEGSPLQ
KLASLKPSSRANGSPTHGRGLHVLQSPAPDEPSFLVTLPTCQMCLEG
NOV15c,
CG52261-02 SEQ ID NO: 215 937 bp
DNA Sequence ORF Start: ATG at 317 ORF Stop: TAG at 644
TCAGTCCTGGTCCCTCCCCTTCTTGGGTTCCTCATCCTGCTCTTCTAAATGTCGAGATGCCTGCAGCA
GTTACGCTTATCTCTGGCCACTATCTCTGCTTTTATCTCCTTTCTTAAAAGTCTTCAATGTCTCTAGG
CTGGTGTGTAAAGTCCTCTATCTTCAGTTACTACACCCTTTTCACCTTCAAAATCCTATGCGCACCTC
AAACTCAGCAAGTGTTAACTGAATTAGTCATCTTTGCTGCCATCGGCTGCCAACCTCCACTGTGGCCT
ACTGTGTGTTTCAAAGATGCCTCCGGAAATTATTCCCGTCCCACATGCTCTTTTGCAACGTGACCCTG
CCATCCCCAATGACAGTGGGAGTCCAATCCTCTCCTCTTGAATCTGGGCTGGCTCTTAGGACTCTTGT
CACCAAAAGGATGTGGCAGAAGTGGCACTGTTCAACTTTTGAGGCTAGGCTGAAAAGACTGTACAGCT
TTCTCCTGGTTCTACTAGAAGGCTCCCCCCTACAGAAGCTCGCCTCTCTCAAACCCAGCAGCCGTGCC
AATGGCAGCCCAACGCACAGGAGAGGCTTGCATGTGCTTCAGTCACCAGCTCCAGATGAGCCCAGTTT
TCTGGTAACACTTCCCACCTGTCAGATGTGCTAGCGAGGGCACCTCCAGATGACTCCAGTCCTCAGCC
AGCTGAGTCACCTGTCATTTGAATTCTTCCACCTGAGGCTCCCAGACATTGTCACACAGAGACAAGCC
ATCCACCATCTCTGTGCCCCGTCCAAACTCCTGACCCACGCAGTCCATAAGGAAGAGGTTCTATGCAA
CTAAGTTTGCGATGATGTGTTACACAGCAGTACCCACCACACCCAACAAAACCACCAGTGCTTCCTGG
CTCCCTCTGCCTAAGACATGTGTTTCTGCACATCCATTCACACAGCCAAGAAG
NOV15c,
CG52261-02
Protein Sequence SEQ ID NO: 216 109 aa MW at 12122.2 kD
MLFCNVTLPSPMTVGVQSSPLESGLALRTLVTKRMWQKWHCSTFEARLKRLYSFLLVLLEGSPLQKLA
SLKPSSRANGSPTHRRGLHVLQSPAPDEPSFLVTLPTCQMC
NOV15d,
13382342 SNP
for CG52261-01 SEQ ID NO: 217 937 bp SNP: 347 C/T
DNA Sequence ORF Start: ATG at 317 ORF Stop: end of sequence
TCAGTCCTGGTCCCTCCCCTTCTTGGGTTCCTCATCCTGCTCTTCTAAATGTCGAGATGCCTGCAGCAGT
TACGCTTATCTCTGGCCACTATCTCTGCTTTTATCTCCTTTCTTAAAAGTCTTCAATGTCTCTAGGCTGG
TGTGTAAAGTCCTCTATCTTCAGTTACTACACCCTTTTCACCTTCAAAATCCTATGCGCACCTCAAACTC
AGCAAGTGTTAACTGAATTAGTCATCTTTGCTGCCATCGGCTGCCAACCTCCACTGTGGCCTACTGTGTG
TTTCAAAGATGGCTCCGGAAATTATTCCCGTCCCACATGCTCTTTTGCAACGTGACCCTGCCATCCTCAA
TGACAGTGGCAGTCCAATCCTCTCCTCTTGAATCTGGGCTGGCTCTTACCACTCTTGTCACCAAAAGGAT
GTGGCAGAAGTGGCACTGTTCAACTTTTGAGGCTAGGCTGAAAAGACTGTACAGCTTTCTCCTGGTTCTA
CTAGAAGGCTCCCCCCTACAGAAGCTCGCCTCTCTCAAACCCAGCAGCCGTGCCAATGGCAGCCCAACGC
ACAGGAGAGGCTTGCATGTGCTTCAGTCACCAGCTCCAGATGAGCCCAGTTTTCTGGTAACACTTCCCAC
CTGTCAGATGTGCTAGCGAGGGCACCTCCAGATGACTCCAGTCCTCAGCCAGCTGAGTCACCTGTCATTT
GAATTCTTCCAGCTGAGGCTCCCAGACATTGTCAGACACAGACAAGCCATCCACCATCTCTGTGCCCCGT
CCAAACTCCTGACCCACGCAGTCCATAAGGAAGAGGTTCTATGCAACTAAGTTTGGGATGATGTGTTACA
CAGCAGTACCCACCACACCCAACAAAACCACCAGTGCTTCCTGGCTCCCTCTGCCTAAGACATGTGTTTC
TGCACATCCATTCACACAGCCAAGAAG
NOV15d,
13382342 SNP
for CG52261-01 SNP: Pro to Ser
Protein Sequence SEQ ID NO: 218 109 aa at position 11
MLFCNVTLPSSMTVGVQSSPLESGLALRTLVTKRMWQKWHCSTFEARLKRLYSFLLVLLEGSPLQAAASL
KPSSRANGSPTHRRGLHVLQSPAPDEPSFLVTLPTCQMC
NOV15c,
13382341 SNP
for CG52261-01 SEQ ID NO: 219 937 bp SNP: 563 A/G
DNA Sequence ORF Start: ATG at 317 ORF Stop: end of sequence
TCAGTCCTGGTCCCTCCCCTTCTTGGGTTCCTCATCCTGCTCTTCTAAATGTCGAGATGCCTCCAGCAGT
TACGCTTATCTCTGGCCACTATCTCTGCTTTTATCTCCTTTCTTAAAAGTCTTCAATGTCTCTAGGCTGG
TGTGTAAAGTCCTCTATCTTCAGTTACTACACCCTTTTCACCTTCAAAATCCTATGCGCACCTCAAACTC
AGCAAGTGTTAACTGAATTAGTCATCTTTGCTGCCATCGGCTGCCAACCTCCACTGTGGCCTACTGTGTG
TTTCAAAGATGGCTCCGGAAATTATTCCCGTCCCACATGCTCTTTTGCAACGTGACCCTGCCATCCCCAA
TGACAGTGGGAGTCCAATCCTCTCCTCTTGAATCTGGGCTGGCTCTTAGGACTCTTGTCACCAAAAGGAT
GTGGCAGAAGTGGCACTGTTCAACTTTTGAGGCTAGGCTGAAAAGACTGTACAGCTTTCTCCTGGTTCTA
CTAGAAGGCTCCCCCCTACAGAAGCTCGCCTCTCTCAAACCCAGCAGCCGTGCCAATGGCAGCCCAACCC
ACGGGAGAGGCTTGCATGTGCTTCAGTCACCAGCTCCAGATGAGCCCAGTTTTCTGGTAACACTTCCCAC
CTGTCAGATGTGCTAGCGAGGGCACCTCCAGATGACTCCAGTCCTCAGCCAGCTGAGTCACCTGTCATTT
GAATTCTTCCACCTGAGGCTCCCACACATTGTCAGACAGAGACAAGCCATCCACCATCTCTGTGCCCCGT
CCAAACTCCTGACCCACGCAGTCCATAAGGAAGAGGTTCTATGCAACTAAGTTTGGGATGATGTGTTACA
CAGCAGTACCCACCACACCCAACAAAACCACCAGTGCTTCCTGGCTCCCTCTGCCTAAGACATGTGTTTC
TGCACATCCATTCACACAGCCAAGAAG
NOV15e,
13382341 SNP
for CG52261-01 SNP: Arg to Gly
Protein Sequence SEQ ID NO: 220 109 aa at position 83
MLFCNVTLPSPMTVGVQSSPLESGLALRTLVTKRMWQKWHCSTFEARLKRLYSFLLVLLEGSPLQKLASL
KPSSRANGSPTHGRGLHVLQSPAPDEPSFLVTLPTCQMC

A ClustalW comparison of the above protein sequences yields the following sequence alignment shown in Table 15B.

TABLE 15B
Comparison of the NOV15 protein sequences.

| | |
|---|---|
| NOV15a | ---MLFCNVTLPSPMTVGVQSSPLESGLALRTLVTKRMWQKWHCSTFEAALAALYSFLLV |
| NOV15b | TGSMLFCNVTLPSPMTVGVQSSPLESGLALRTLVTKAAWQKWHCSTFEARLKRLYSFLLV |
| NOV15c | ---MLFCNVTLPSPMTVGVQSSPLESGLALRTLVTKRMWQKWHCSTFEARLAALYSFLLV |
| NOV15a | LLEGSPLQKLASLKPSSRANGSPTHRRGLHVLQSPAPDEPSFLVTLPTCQMC--- |
| NOV15b | LLEGSPLQKLASLKPSSRANGSPTHGRGLHVLQSPAPDEPSFLAALPTCQMCLEG |
| NOV15c | LLEGSPLQKLASLKPSSRANGSPTHRRGLHVLQSPAPDEPSFLVTLPTCQMC--- |
| NOV15a | (SEQ ID NO: 212) |
| NOV15b | (SEQ ID NO: 214) |
| NOV15c | (SEQ ID NO: 216) |

Further analysis of the NOV15a protein yielded the following properties shown in Table 15C.

TABLE 15C
Protein Sequence Properties NOV15a

| SignalP | Cleavage site between residues 65 and 66 | | | |
|---|---|---|---|---|
| analysis: | | | | |
| PSORT II | PSG: a new signal peptide prediction method | | | |
| analysis: | N-region: length 0; pos. chg 0; neg. chg 0 | | | |
| | H-region: length 21; peak value 7.16 | | | |
| | PSG score: 2.76 | | | |
| | GvH: von Heijne's method for signal seq. recognition | | | |
| | GvH score (threshold: −2.1): −5.57 | | | |
| | possible cleavage site: between 18 and 19 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 0 | | | |
| | number of TMS(s) . . . fixed | | | |
| | PERIPHERAL | Likelihood = | 3.18 (at 54) | |
| | ALOM score: 3.18 (number of TMSs: 0) | | | |
| | MTOP: Prediction of membrane topology (Hartmann et al.) | | | |
| | Center position for calculation: 6 | | | |
| | Charge difference: 1.0 C(2.0)-N(1.0) | | | |
| | C > N: C-terminal side will be inside | | | |
| | >>>Caution: Inconsistent mtop result with signal peptide | | | |
| | MITDISC: discrimination of mitochondrial targeting seq | | | |
| | R content: | 0 | Hyd Moment (75): | 5.42 |
| | Hyd Moment (95): | 2.82 | G content: | 1 |
| | D/E content: | 1 | S/T content: | 5 |

```
Score: −4.44
Gavel: prediction of cleavage sites for mitochondrial preseq
R-3 motif at 53 KRLY|S
NUCDISC: discrimination of nuclear localization signals
pat4: none
pat7: none
bipartite: none
content of basic residues: 11.0%
NLS Score: −0.47
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: nuclear
Reliability: 89
COIL: Lupas's algorithm to detect coiled-coil regions
total: 0 residues
Final Results (k = 9/23):
65.2%: nuclear
21.7%: mitochondrial
  8.7%: cytoplasmic
  4.3%: peroxisomal
>> prediction for CG52261-01 is nuc (k = 23)
```

A search of the NOV15a protein against the Geneseq database, a proprietary database that contains sequences published in patents and patent publication, yielded several homologous proteins shown in Table 15D.

TABLE 15D
Geneseq Results for NOV15a

| | | NOV15a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAB85225 | Human secreted protein (SECX) | 1 . . . 109 | 109/109 (100%) | 2e−59 |
| | sequence (clone 3277237) - Homo | 1 . . . 109 | 109/109 (100%) | |
| | sapiens, 109 aa. [WO200144287-A2, | | | |
| | 21 JUN. 2001] | | | |
| AAB14303 | Human secreted protein encoded by | 1 . . . 109 | 109/109 (100%) | 2e−59 |
| | cDNA clone 3277237 - Homo | 1 . . . 109 | 109/109 (100%) | |
| | sapiens, 109 aa. [WO200037634-A2, | | | |
| | 29 JUN. 2000] | | | |
| AAB46696 | Fowlpox virus DNA polymerase | 2 . . . 57 | 15/56 (26%) | 3.9 |
| | protein fragment SEQ ID NO 5 - | 448 . . . 503 | 34/56 (59%) | |
| | Fowlpox virus, 874 aa. | | | |
| | [WO200075335-A2, 14 DEC. 2000] | | | |
| AAU41849 | Propionibacterium acnes | 10 . . . 40 | 14/34 (41%) | 3.9 |
| | immunogenic protein #2745 - | 33 . . . 66 | 20/34 (58%) | |
| | Propionibacterium acnes, 109 aa. | | | |
| | [WO200181581-A2, 01 NOV. 2001] | | | |
| AAO16328 | Human polyamine oxidases (PAO) | 52 . . . 97 | 17/50 (34%) | 6.7 |
| | isoform 4 - Homo sapiens, 532 aa. | 429 . . . 478 | 27/50 (54%) | |
| | [WO2002100884-A2, 19 DEC | | | |
| | 2002] | | | |

In a BLAST search of public sequence databases, the NOV15a protein was found to have homology to the proteins shown in the BLASTP data in Table 15E.

TABLE 15E
Public BLASTP Results for NOV15a

| | | NOV15a | Identities/ | |
|---|---|---|---|---|
| Protein | | Residues/ | Similarities for | |
| Accession | | Match | the Matched | |
| Number | Protein/Organism/Length | Residues | Portion | Expect Value |
| CAC50793 | Sequence 7 from Patent | 1 . . . 109 | 109/109 (100%) | 7e−59 |
| | WO0144287 - Homo sapiens | 1 . . . 109 | 109/109 (100%) | |
| | (Human), 109 aa. | | | |

### Example 16

The NOV16 clone was analyzed, and the nucleotide and encoded polypeptide sequences are shown in Table 16A.

TABLE 16A
NOV16 Sequence Analysis

NOV16a,
CG52414-02 SEQ ID NO: 221 3040 bp
DNA Sequence ORF Start: ATG at 338 ORF Stop: TGA at 2819
TTTGGGGCCGCAGGGAGGTTCCCAGACCAGAGGACTGTTGTTAGGTGATTGGCTGTGAACGCCCTCAG
GCCAGTGCCCCTCGCTGCTTGGCACTCGGAGATGCCTGATTAGCACCTTTAATCCCTTACCAATGAGG
CAGGTGGAATTGGCCCCATTTTACAGATGGGGAGACTGAGCCACCTGTCTGTCCAGCCACCCTTCCAC
AGACTGAGGCTTGACACCGGAGCATCTGTACAGAGCAAGGAGAAGACAAGAACATGCTCTAAAGCCCT
TCACAGCAAGACCCAGGAAGCCGCGGGCAAACTCAGACTCGAAGCCCTCCCACCTCCTGCCCACAATG
GCCTCTGCTGACAAGAATGGCGGGAGCGTGTCCTCTGTGTCCAGCAGCCGCCTGCAGAGCCGGAAGCC
ACCCAACCTCTCCATCACCATCCCGCCACCCGAGAAAGAGACCCAGGCCCCTGGCCAGCAGGACAGCA
TGCTGCCTGAGAGGAAGAACCCAGCCTACTTGAACAGCGTCAGCCTCCAGGAGCCACGCAGCCGATGG
CAGGAGAGTTCAGAGAAGCGCCCTGGCTTCCGCCGCCAGGCCTCACTGTCCCAGAGCATCCGCAAGGG
CGCAGCCCAGTGGTTTGGAGTCAGCGGCGACTGGGAGGGGCACCGGCAGCAGTGGCAGCGCCGCAGCC
TGCACCACTGCAGCATGCGCTACGGCCGCCTGAAGGCCTCGTGCCAGCGTGACCTGGAGCTCCCCAGC
CAGGAGGCACCGTCCTTCCAGGGCACTGAGTCCCCAAAGCCCTGCAAGATGCCCAAGATTGTGGATCC
GCTGGCCCGGGGCCGGCCCTTCCGCCACCCGGAGGAGATGGACAGGCCCCACGCCCCGCACCCACCGC
TGACCCCCGGAGTCCTGTCCCTCACCTCCTTCACCAGTGTCCGTTCTCGCTACTCCCACCTGCCACGC
CGCAAGAGAATGTCTGTGGCCCACATGAGCTTGCAAGCTGCCGCTGCCCTCCTCAAGGGGCGCTCGGT
GCTGGATGCCACCGGACAGCGGTGCCGGGTGGTCAAGCGCAGCTTTCCCTTCCCGAGCTTCCTGGACG
AGGATGTGGTCGATGGGGCAGACACGTTTGACTCCTCCTTTTTTAGTAAGGAAGAAATGAGCTCCATG
CCTGATGATGTCTTTGAGTCCCCCCCACTCTCTCCCAGCTACTTCCGAGGGATCCCACACTCAGCCTC
CCCTGTCTCCCCCGATGGGGTCCAAATCCCTCTGAAGGAGTATGGCCGAGCCCCAGTCCCCGCGCCCC
GGCCCGGCAAGCGCATCGCCTCCAAGGTGAAGCACTTTGCCTTTGATCGGAAGAAGCGGCACTACGGC
CTCGGCGTGGTGGGCAACTGGCTGAACCGCAGCTACCGCCGCAGCATCAGCAGCACTGTGCAGCGGCA
GCTGGAGAGCTTCGACAGCCACCGGCCCTACTTCACCTACTGGCTGACCTTCGTCCATGTCATCATCA
CGCTGCTGGTGATTTGCACGTATGGCATCGCACCCGTGGCCTTTGCCCAGCACGTCACCACCCAGCTG
GTGCTGCCGAACAAAGGTGTGTACGAGACCGTGAAGTACATCCAGCAGGAGAACTTCTGGGTTGGCCC
CAGCTCGATTGACCTGATCCACCTGGGGGCCAAGTTCTCACCCTGCATCCGCAAGGACGGGCAGATCG
AGCAGCTGGTGCTGCGCGAGCGAGACCTGGAGCGGGACTCAGGCTGCTGTGTCCAGAATGACCACTCC
GGATGCATCCAGACCCAGCGGAAGGACTGCTCGGACACTTTGGCCACTTTTGTCAAGTGGCAGGATGA
CACTGGGCCCCCCATGGACAAGTCTGATCTGGGCCAGAAGCGGACTTCGGGGGCTGTCTGCCACCAGG
ACCCCAGGACCTGCGAGGAGCCAGCCTCCAGCGGTGCCCACATCTGGCCCGATGACATCACTAAGTGG
CCCATCTGCACAGAGCAGGCCAGGAGCAACCACACAGGCTTCCTGCACATGGACTGCGAGATCAAGGG
CCGCCCCTGCTGCATCGGCACCAAGGGCAGCTGTGAGATCACCACCCGGGAATACTGTGAGTTCATGC
ACGGCTATTTCCATGAGGAAGCAACACTCTGCTCCCAGGTGCACTGCTTGGACAACGTGTGTGGGCTG
CTGCCCTTCCTCAACCCTGAGGTCCCAGATCAGTTCTACAGGCTCTGGCTGTCTCTCTTCCTACATGC
TGGGGTGGTGCACTGCCTCGTGTCTGTGGTCTTTCAAATGACCATCCTGAGGGACCTGGAGAAGCTGG
CCGGCTGCCACCGTATCGCCATCATCTTCATCCTCAGTGGCATCACAGGCAACCTCGCCAGTGCCATC
TTTCTCCCATACCGGGCAGAGGTAGGCCCGGCCGGCTCACAGTTCGGCCTCCTCGCCTGCCTCTTCGT
GGAGCTCTTCCAGAGCTGGCCGCTGCTGGAGAGGCCCTGGAAGGCCTTCCTCAACCTCTCGGCCATCG
TGCTCTTCCTGTTCATCTGTGGCCTCCTGCCCTGGATCGACAACATCGCCCACATCTTCGGCTTCCTC
AGTGGCCTGCTGCTGGCCTTCGCCTTCCTGCCCTACATCACCTTCGGCACCAGCGACAAGTACCGCAA
CCGGGCACTCATCCTGGTGTCACTGCTGGCCTTTGCCGGCCTCTTCGCCGCCCTCGTGCTGTGGCTGT
ACATCTACCCCATTAACTGGCCCTGGATCGAGCACCTCACCTGCTTCCCCTTCACCAGCCGCTTCTGC
GAGAAGTATGAGCTGGACCAGGTGCTCCACTGACCGCTGGGCCACACGGCTGCCCCTCAGCCCTGCTG
GAACAGGGTCTGCCTGCGAGGGCTGCCCTCTGCAGAGCGCTCTCTGTGTGCCAGAGAGCCAGAGACCC
AAGACAGGGCCCGGGCTCTGGACCTGGGTGCCCCCCTGCCAGGCGAGGCTGACTCCGCGTGACATAGA
TGGTTGGTTAAGGCGGGGTTTTTCCGGGCCGCGCCCCCCCCCTCTAAA
NOV16a,
CG52414-02
Protein Sequence SEQ ID NO: 222 827 aa MW at 93378.2 kD
MASADKNGGSVSSVSSSRLQSRKPPNLSITIPPPEKETQAPGEQDSMLPERKNPAYLKSVSLQEPRSR
WQESSEKRPGFRRQASLSQSIRKGAAQWFGVSGDWEGQRQQWQRRSLHNCSMRYGRLKASCQRDLELP
SQEAPSFQGTESPKPCKMPKIVDPLARGRAFRHPEEMDRPHAPHPPLTPGVLSLTSFTSAASGYSHLP
RRKRMSVAHMSLQAAAALLKGRSVLDATGQRCRVVKRSFAFPSFLEEDVVDGADTFDSSFFSKEEMSS
MPDDVFESPPLSASYFRGIPHSASPVSPDGVQIPLKEYGRAPVPGPRRGKRIASKVKHFAFDRKKRHY
GLGVVGNWLNRSYRRSISSTVQRQLESFDSHRPYFTYWLTFVHVILTLLVICTYGIAPVGFAQHVTTQ
LVLRNKGVYESVKYIQQENFWVGPSSIDLIHLGAKFSPCIRKDGQIEQLVLRERDLERDSGCCVQNDH
SGCIQTQRKDCSETLATFVKWQDDTGPPMDKSDLGQKRTSGAVCHQDPRTCEEPASSGAHIWPDDITK
WPICTEQARSNHTGFLHMDCEIKGRPCCIGTKGSCEITTREYCEFMHGYFHEEATLCSQVHCLDKVCG
LLPFLNPEVPDQFYRLWLSLFLHAGVVHCLVSVVFQMTILRDLEAAAGWHRIAIIFILSGTTGNLASA
IFLPYRAEVGPAGSQFGLLACLFVELFQSWPLLERPWKAFLNLSAIVLFLFICGLLPWIDNIAHIFGF
LSGLLLAFAFLPYITFGTSDKYRKRALILVSLLAFAGLFAALVLWLYIYPINWPWIEHLTCFPFTSRF
CEKYELDQVLH
NOV16b,
305262879 SEQ ID NO: 223 694 bp
DNA Sequence ORF Start: at 2 ORF Stop: end of sequence
CACCAGATCTCAGCACGTCACCACCCAGCTGGTGCTGCGGAACAAAGGTGTGTACGAGAGCGTGAAGT
ACATCCAGCAGGAGAACTTCTGGGTTGGCCCCAGCTCGATTGACCTGATCCACCTGGGGGCCAAGTTC
TCACCCTGCATCCGGAAGGACGGGCAGATCGAGCAGCTGGTCCTGCGCGAGCGAGACCTGGAGCGGGA
CTCAGGCTGCTGTGTCCAGAATGACCACTCCGGATGCATCCAGACCCAGCGGAAGGACTGCTCGGACA
CTTTGGCCACTTTTGTCAAGTGGCAGGATGACACTGGGCCCCCCATGGACAAGTCTGATCTGGGCCAG
AAGCGGACTTCGGGGGCTGTCTGCCACCAGGACCCCAGGACCTGCGAGGAGCCAGCCTCCAGCGGTGC
CCACATCTGGCCCGATGACATCACTAAGTGGCCGATCTGCACAGAGCAGGCCAGGAGCAACCACACAG
GCTTCCTGCACATGGACTGCGAGATCAAGGGCCGCCCCTGCTGCATCGGCACCAAGGGCAGCTGTGAG
ATCACCACCCGGGAATACTGTGAGTTCATGCACGGCTATTTCCATCAGGAAGCAACACTCTGCTCCCA
GGTGCACTGCTTGGACAAGGTGTGTGGGCTCCTGCCCTTCCTCAACCCTGAGGTCCCAGATCAGTTCT
ACAGGCTCGAGGGC
NOV16b,
305262879
Protein Sequence SEQ ID NO: 224 231 aa MW at 26183.3 kD
TRSQHVTTQLVLRNKGVYESVKYIQQENFWVGPSSIDLIHLGAKFSPCIRKDGQIEQLVLRERDLERD
SGCCVQNDHSGCIQTQRKDCSETLATFVKWQDDTGPPMDKSDLGQKRTSGAVCHQDPRTCEEPASSGA
HIWPDDITKWPICTEQARSNHTGFLHMDCEIKGRPCCIGTKGSCEITTREYCEFMHGYFHEEATLCSQ
VHCLDKVCGLLPFLNPEVPDQFYRLEG
NOV16c,
319073326 SEQ ID NO: 225 2506 bp
DNA Sequence ORF Start: at 2 ORF Stop: end of sequence
CACCAGATCTCCCACCATGGCCTCTCCTGACAAGAATGGCGGGAGCGTGTCCTCTGTGTCCAGCAGCC
GCCTGCAGAGCCGGAAGCCACCCAACCTCTCCATCACCATCCCGCCACCCGAGAAAGAGACCCAGGCC
CCTGGCGAGCAAGACAGCATGCTGCCTGAGAGGAAGAACCCAGCCTACTTGAAGAGCGTCAGCCTCCA
GGAGCCACGCAGCCCATGGCAGGAGAGTTCAGAGAAGCGCCCTGGCTTCCGCCGCCAGGCCTCACTGT
CCCAGAGCATCCGCAAGGGCGCAGCCCAGTGGTTTGGAGTCAGCGGCGACTGGGAGGGGCAGCGGCAG
CAGTGGCAGCGCCGCAGCCCGCACCACTGCAGCATGCGCTACGGCCGCCTGAAGGCCTCGTGCCAGCG
TGACCTGGAGCTCCCCAGCCAGGAGGCACCGTCCTTCCAGGGCACTGAGTCCCCAAAGCCCTGCAAGA
TGCCCAAGATTGTGGATCCGCTGCCCCGGGGCCGGGCCTTCCGCCACCCGGAGGAGATGGACAGGCCC
CACGCCCTGCACCCACCGCTGACCCCCGGAGTCCTGTCCCTCACCTCCTTCACCAGTGTCCGTTCTGG
CTAcTCCCACCTGCCACGCCGCAAGAGAATGTCTGTGGCCCACATGAGCTTGCAAGCTGCCGCTGCCC
TCCTCAAGGGGCGCTCGGTGCTGGATGCCACCGGACAGCGGTGCCGGATGGTCAAGCGCAGCTTTGCC
TTCCCGAGCTTCCTGGAGGAGGATGTGGTCGATGGGGCACACACGTTTGACTCCTCCTTTTTTAGTAA
GGAAGAAATGAGCTCCATGCCTGATGATGTCTTTGAGTCCCCCCCACTCTCTGCCAGCTACTTCCGAG
GGATCCCACACTCAGCCTCCCCTGTCTCCCCCGATGGGGTGCAAATCCCTCTGAAGGAGTATGGCCGA
GCCCCAGTCCCCGGGCCCCGGCGCGGTGAGCGCATCGCCTCCAAGGTGAAGCACTTTGCCTTTGATCG
GAAGAAGCGGCACTACGGCCTCGCCGTGGTGGGCAACTGGCTGAACCGCAGTTACCCCCGCAGCATCA
GCAGCACTGTGCACCGGCAGCTGGAGAGCTTCGACAGCCACCGGCCCTACTTCACCTACTGGCTGACC
TTCGTCCATCTCATCATCACGCTGCTGGTGATTTGCACGTATGGCATCGCACCCGTGGGCTTTGCCCA
GCACGTCACCACCCAGCTGGTGCTGCGGAACAAAGGTGTGTACGAGAGCGTGAAGTACATCCAGCAGG
AGAACTTCTGGGTTGGCCCCAGCTCGATTGACCTGATCCACCTGGGGGCCAAGTTCTCACCCTGCATC
CGGAACGACGGGCAGATCGAGCAGCTGGTGCTGCGCGAGCGAGACCTGGAGCGGGACTCACGCTGCTG
TGTCCAGAATGACCACTCCGGATGCATCCAGACCCAGCGGAAGGACTGCTCGGAGACTTTGGCCACTT
TTGTCAAGTGGCAGGATGACACTGGGCCCCCCATCCACAAGTCTGATCTGGGCCAGAAGCGGACTTCG
GGGGCTGTCTGCCACCAGGACCCCAGGACCTGCGAGGAGCCAGCCTCCAGCGGTGCCCACATCTGGCC
CGATGACATCACTAAGTGGCCGATCTGCACAGAGCAGGCCAGGAGCAACCACACAGGCTTCCTGCACA
TGGACTGCGAGATCAAGGGCCGCCCCTGCTGCATCGGCACCAAGGGCAGCTGTGAGATCACCACCCGG
GAATACTGTGAGTTCATGCACCGCTATTTCCATGAGGAAGCAACACTCTGCTCCCAGGTCCACTGCTT
GGACAAGGTGTGTGGGCTCCTCCCCTTCCTCAACCCTGAGGTCCCAGATCAGTTCTACACGCTCTGGC
TGTCTCTCTTCCTACATGCTGGCGTGGTGCACTGCCTCGTGTCTGTGGTCTTTCAAATGACCATCCTG
AGGGACCTGGACAAGCTGGCCGGCTGGCACCGTATCGCCATCATCTTCATCCTCAGTGGCATCACAGG
CAACCTCGCCAGTGCCATCTTTCTCCCATACCGGGCAGAGGTGGGCCCGGCCGGCTCACAGTTCGGCC
TCCTCGCCTGCCTCTTCGTGGAGCTCTTCCAGAGCTGGCCGCTGCTGGAGAGGCCCTGGAAGGCCTTC
CTCAACCTCTCGGCCATCGTGCTCTTCCTGTTCATCTGTGGCCTCCTGCCCTGGATCGACAACATCGC
CCACATCTTCGGCTTCCTCAGTGGCCTGCTGCTGGCCTTCGCCTTCCTGCCCTACATCACCTTCGGCA
CCAGCGACAAGTACCGCAAGCGGGCACTCATCCTGGTGTCACTGCTGGCCTTTGCCGGCCTCTTCGCC
GCCCTCGTGCTGTGGCTGTACATCTACCCCATTAACTGGCCCTGGATCGAGCACCTCACCTGCTTCCC
CTTCACCACCCGCTTCTGCGAGAAGTATGACCTGGACCAGGTGCTGCACCTCGAGGGC
NOV16c,
319073326
Protein Sequence SEQ ID NO: 226 835 aa MW at 94253.1 kD
TRSPTMASADKNGGSVSSVSSSRLQSRKPPNLSITIPPPEKETQAPGEQDSMLPERKNPAYLKSVSLQ
EPRSRWQESSEKRPGFRRQASLSQSIRKGAAQWFGVSGDWEGQRQQWQRRSPHHCSMRYGRLKASCQR
DLELPSQEAPSFQGTESPKPCKMPKIVDPLARGRAFRHPEEMDRPHALHPPLTPGVLSLTSFTSVRSG
YSHLPRRKRMSVAHMSLQAAAALLKGRSVLDATGQRCRMVKRSFAFPSFLEEDVVDGADTFDSSFFSK
EEMSSMPDDVFESPPLSASYFRGIPHSASPVSPDGVQIPLKEYCRAPVPGPRRGERIASKVKHFAFDR
KKRHYGLGVVGNWLNRSYRRSISSTVQRQLESFDSHRPYFTYWLTFVHVIITLLVTCTYGIAPVGFAQ
HVTTQLVLRNKGVYESVKYIQQENFWVGPSSIDLIHLGAKFSPCIRKDGQIEQLVLRERDLERDSGCC
VQNDHSGCIQTQRKDCSETLATFVKWQDDTGPPMDKSDLGQKRTSGAVCHQDPRTCEEPASSGAHIWP
DDITKWPICTEQARSNHTGFLHMDCEIKGRPCCIGTKGSCEITTREYCEFMHGYFHEEATLCSQVHCL
DKVCGLLPFLNPEVPDQFYRLWLSLFLHAGVVHCLVSVVFQMTILRDLEKLAGWHRIAIIFILSGITG
NLASAIFLPYRAEVGPAGSQFGLLACLFVELFQSWPLLERPWKAFLNLSAIVLFLFICGLLPWIDNIA
HIFGFLSGLLLAFAFLPYITFGTSDKYRKRALILVSLLAFAGLFAALVLWLYIYPINWPWIEHLTCFP
FTSRFCEKYELDQVLHLEG
NOV16d,
CG52414-01 SEQ ID NO: 227 2596 bp
DNA Sequence ORF Start: at 289 ORF Stop: TGA at 2413
TCAATTGACTTGATATGATTTATTATTTTTACTACTTATAAGAATGCAAATAAGTTCTCCTTAGTTTT
TTTCTTGGAGAAAGTCTGACATGTGAGGCACAGATGAGTTATTAAAGGCAGATGACTTTCCAGCCTTG
TCTTAAATGTTCCATTCTTTACCTTAGAAATTATTTAAATTTGTGTCCTGTCCCAGAGCATCCGCAAG
GGCGCAGCCCAGTGGTTTGGAGTCAGCGGCGACTGGGAGGGGCAGCGGCAGCAGTGGCAGCGCCGCAG
CCTGCACCACTGCAGCATGCGCTACGGCCGCCTGAAGGCCTCGTGCCAGCGTGACCTGGAGCTCCCCA
GCCAGGAGGCACCGTCCTTCCAGGGCACTGAGTCCCCAAAGCCCTGCAAGATGCCCAAGATTGTGGAT
CCGCTGGCCCGGGGCCGGGCCTTCCGCCACCCGGAGGAGATGGACAGGCCCCACGCCCTGCACCCACC
GCTGACCCCCGGAGTCCTGTCCCTCACCTCCTTCACCAGTGTCCGTTCTGGCTACTCCCACCTGCCAC
GCCGCAAGAGAATGTCTGTGGCCCACATGAGCTTGCAAGCTGCCGCTGCCCTCCTCAAGGGGCGCTCG
GTGCTGGATGCCACCGGACAGCGGTGCCGGGTGGTCAAGCGCAGCTTTGCCTTCCCGAGCTTCCTGGA
GGAGCATGTGGTCGATGGGGCAGACACCTTTGACTCCTCCTTTTTTAGTAAGGAAGAAATGAGCTCCA
TGCCTGATGATGTCTTTGAGTCCCCCCCACTCTCTGCCAGCTACTTCCGAGGGATCCCACACTCAGCC
TCCCCTGTCTCCCCCGATGGGGTGCAAATCCCTCTGAAGGAGTATGGCCGAGCCCCAGTCCCCGGGCC
CCGGCGCGGCAAGCGCATCGCCTCCAAGGTGAAGCACTTTGCCTTTGATCGGAAGAAGCGGCACTACG
GCCTCGGCGTGGTGGGCAACTGGCTGAACCGCAGCTACCGCCGCAGCATCAGCAGCACTGTGCAGCGG
CAGCTGGAGAGCTTCGACAGCCACCGGCCCTACTTCACCTACTGGCTGACCTTCGTCCATGTCATCAT
CACGCTGCTGGTGATTTGCACGTATCGCATCGCACCCGTGGGCTTTGCCCAGCACGTCACCACCCAGC
TGGTGCTGCGGAACAAAGGTGTGTACGAGAGCGTGAAGTACATCCAGCAGGAGAACTTCTGGGTTGGC
CCCAGCTCGATTGACCTGATCCACCTGGGGGCCAAGTTCTCACCCTGCATCCGGAAGGACGGGCAGAT
CGAGCAGCTGGTGCTGCGCGAGCGAGACCTGGAGCGGCACTCAGGCTGCTGTGTCCAGAATGACCACT
CCGGCTGCATCCAGACCCAGCGGAAGGACTGCTCGGAGACTTTGGCCACTTTTGTCAAGTGGCAGGAT
GACACTGGGCCCCCCATGGACAAGTCTGATCTGGGCCAGAAGCGGACTTCGGGGGCTGTCTGCCACCA
GGACCCCAGGACCTGCGAGGAGCCAGCCTCCAGCGGTGCCCACATCTGGCCCGATGACATCACTAAGT
GGCCGATCTGCACAGAGCAGGCCAGGAGCAACCACACAGGCTTCCTGCACATGGACTGCGAGATCAAG
GGCCGCCCCTGCTGCATCGGCACCAAGGGCAGCTGTGAGATCACCACCCGGGAATACTGTGAGTTCAT
GCACGGCTATTTCCATGAGGAACCAACACTCTGCTCCCAGGTGCACTGCTTGGACAAGGTGTGTGGGC
TGCTGCCCTTCCTCAACCCTCACCTCCCAGATCAGTTCTACAGGCTCTGGCTGTCTCTCTTCCTACAT
GCTGGCGTGGTGCACTGCCTCGTGTCTGTGGTCTTTCAAATGACCATCCTCAGGGACCTGGAGAAGCT
GGCCGGCTGGCACCGTATCGCCATCATCTTCATCCTCAGTGGCATCACAGGCAACCTCGCCAGTACCA
TCTTTCTCCCATACCGCGCAGAGGTGGGCCCGGCCGGCTCACAGTTCGGCCTCCTCCCCTGCCTCTTC
GTGGAGCTCTTCCAGAGCTGGCCGCTGCTGGAGAGGCCCTGGAAGGCCTTCCTCAACCTCTCGACCAT
CGTGCTCTTCCTGTTCATCTGTCGCCTCCTGCCCTGGATCGACAACATCGCCCACATCTTCGGCTTCC
TCAGTGCCCTGCTGCTGGCCTTCGCCTTCCTGCCCTACATCACCTTCGGCACCAGCGACAAGTACCGC
AAGCGGGCACTCATCCTGGTGTCACTGCTGGCCTTTGCCGGCCTCTTCGCCGCCCTCGTGCTGTGGCT
GTACATCTACCCCATTAACTGGCCCTGGATCGAGCACCTCACCTGCTTCCCCTTCACCAGCCGCTTCT
GCGAGAAGTATGAGCTGGACCAGGTGCTGCACTGACCGCTGGGCCACACGGCTGCCCCTCAGCCCTGC
TGGAACAGGGTCTGCCTGCGAGGGCTGCCCTCTGCAGAGCGCTCTCTGTGTGCCAGAGAGCCAGAGAC
CCAAGACAGGGCCCGGGCTCTGGACCTGGGTGCCCCCCTCCCAGGCGAGGCTGACTCCGCGTGAGATG
GTTGGTTAAGGC
NOV16d,
CG52414-01
Protein Sequence SEQ ID NO: 228 708 aa MW at 80098.6 kD
MRYGRLKASCQRDLELPSQEAPSFQGTESPKPCKMPKIVDPLARGRAFRHPEEMDRPHALHPPLTPGV
LSLTSFTSVRSGYSHLPRRKRMSVAHMSLQAAAALLKGRSVLDATGQRCRVVKRSFAFPSFLEEDVVD
GADTFDSSFFSKEEMSSMPDDVFESPPLSASYFRGIPHSASPVSPDGVQIPLKEYGRAPVPGPRRGKR
IASKVKKFAFDRKKRHYGLGVVGNWLNRSYRRSISSTVQRQLESFDSHRPYFTYWLTFVHVIITLLVI
CTYGIAPVGFAQHVTTQLVLRNKGVYESVKYIQQENFWVGPSSIDLIHLGAKFSPCIRKDGQIEQLVL
RERDLERDSGCCVQNDHSGCIQTQRKDCSETLATFVKWQDDTGPPMDKSDLGQKRTSCAVCHQDPRTC
EEPASSGAHIWPDDITKWPICTEQARSNHTGFLHMDCEIKGRPCCIGTKGSCEITTREYCEFMHGYFH
EEATLCSQVHCLDKVCGLLPFLNPEVPDQFYRLWLSLFLHAGVVHCLVSVVFQMTILRDLEKLAGWHR
TATIFILSGITGNLASTIFLPYRAEVGPAGSQFGLLACLFVELFQSWPLLERPWKAFLNLSTIVLFLF
ICGLLPWIDNIAHIFGFLSGLLLAFAFLPYITFGTSDKYRKRALTLVSLLAFAGLFAALVLWLYIYPI
NWPWTEHLTCFPFTSRFCEKYELDQVLH
NOV16e,
CG52414-03 SEQ ID NO: 229 2516 bp
DNA Sequence ORF Start: ATG at 17 ORF Stop: end of sequence
CACCAGATCTCCCACCATGGCCTCTGCTGACAAGAATGGCGGGAGCGTGTCCTCTGTGTCCAGCAGCC
GCCTGCAGAGCCGGAAGCCACCCAACCTCTCCATCACCATCCCGCCACCCGAGAAAGAGACCCAGGCC
CCTGGCGAGCAGGACACCATGCTGCCTGAGAGGAAGAACCCAGCCTACTTGAAGAGCGTCAGCCTCCA
GGAGCCACGCACCCGATGGCAGGAGAGTTCAGAGAAGCGCCCTGGCTTCCGCCGCCAGGCCTCACTGT
CCCAGAGCATCCGCAAGGGCGCAGCCCAGTGGTTTGCAGTCAGCGGCGACTGGGAGGGGCAGCGGCAG
CAGTGGCAGCGCCGCAGCCTGCACCACTGCAGCATGCGCTACGGCCGCCTGAAGGCCTCGTGCCAGCG
TGACCTGGAGCTCCCCAGCCAGGAGGCACCGTCCTTCCAGGGCACTGAGTCCCCAAAGCCCTGCAAGA
TGCCCAAGATTGTGGATCCGCTGGCCCGGGGCCGGGCCTTCCGCCACCCGGAGGAGATGGACAGGCCC
CACGCCCCGCACCCACCGCTGACCCCCGGAGTCCTGTCCCTCACCTCCTTCACCAGTGTCCGTTCTGG
CTACTCCCACCTGCCACGCCGCAAGAGAATGTCTGTGGCCCACATGAGCTTGCAAGCTGCCGCTGCCC
TCCTCAAGGGGCGCTCGGTGCTGGATGCCACCGGACAGCGGTGCCGGGTGGTCAAGCGCAGCTTTGCC
TTCCCGAGCTTCCTGGAGGAGGATGTGGTCGATGGGGCAGACACGTTTGACTCCTCCTTTTTTAGTAA
GGAAGAAATGAGCTCCATGCCTGATGATGTCTTTGAGTCCCCCCCACTCTCTGCCAGCTACTTCCGAG
GGATCCCACACTCAGCCTCCCCTGTCTCCCCCGATGGGGTGCAAATCCCTCTGAAGGAGTATGGCCGA
GCCCCAGTCCCCGGGCCCCGGCGCGGCAAGCGCATCGCCTCCAAGGTGAAGCACTTTGCCTTTGATCG
CAAGAAGCGGCACTACGGCCTCGGCGTGGTGGGCAACTGGCTGAACCGCAGCTACCGCCGCAGCATCA
GCAGCACTGTGCAGCGGCAGCTGGAGAGCTTCCACAGCCACCGGCCCTACTTCACCTACTGGCTGACC
TTCGTCCATGTCATCATCACGCTGCTGGTGATTTGCACGTATGGCATCGCACCCGTGGGCTTTGCCCA
GCACGTCACCACCCAGCTGGTGCTGCGGAACAAAGGTGTGTACGAGAGCGTGAAGTACATCCAGCAGG
AGAACTTCTGGGTTGGCCCCAGCTCGATTGACCTGATCCACCTGGGGGCCAAGTTCTCACCCTGCATC
CGGAAGGACGGGCAGATCGAGCAGCTGGTGCTGCGCGAGCGAGACCTGGAGCGGGACTCAGGCTGCTG
TGTCCAGAATGACCACTCCGGATGCATCCAGACCCAGCGGAAGGACTGCTCGGAGACTTTGGCCACTT
TTGTCAAGTGGCAGGATGACACTGGGCCCCCCATGGACAAGTCTGATCTGGGCCAGAACCGGACTTCG
GGCGCTGTCTGCCACCAGGACCCCAGGACCTGCGAGGAGCCAGCCTCCAGCGGTGCCCACATCTGGCC
CGATGACATCACTAAGTGGCCGATCTGCACAGAGCACCCCAGGAGCAACCACACAGGCTTCCTGCACA
TGGACTGCGAGATCAAGGGCCGCCCCTGCTGCATCGGCACCAAGGGCAGCTGTGAGATCACCACCCGG
GAATACTGTGAGTTCATGCACGGCTATTTCCATGAGGAAGCAACACTCTGCTCCCAGGTGCACTGCTT
GGACAAGGTCTGTGGGCTGCTGCCCTTCCTCAACCCTGAGGTCCCAGATCAGTTCTACAGGCTCTGGC
TGTCTCTCTTCCTACATGCTGGCGTGGTGCACTGCCTCGTGTCTGTGGTCTTTCAAATGACCATCCTG
AGGGACCTGGAGAAGCTGGCCGGCTGGCACCGTATCGCCATCATCTTCATCCTCAGTCGCATCACAGG
CAACCTCGCCAGTGCCATCTTTCTCCCATACCGGGCAGAGGTGGGCCCGGCCGGCTCACAGTTCGGCC
TCCTCGCCTGCCTCTTCGTGGAGCTCTTCCAGAGCTGGCCGCTGCTGGAGAGGCCCTGGAAGGCCTTC
CTCAACCTCTCGGCCATCGTGCTCTTCCTGTTCATCTGTGGCCTCCTGCCCTGGATCGACAACATCGC
CCACATCTTCGGCTTCCTCAGTGGCCTGCTGCTGGCCTTCGCCTTCCTGCCCTACATCACCTTCGGCA
CCAGCGACAAGTACCGCAAGCGGGCACTCATCCTGGTGTCACTGCTGGCCTTTGCCGGCCTCTTCGCC
GCCCTCGTGCTGTGGCTGTACATCTACCCCATTAACTGCCCCTGGATCGAGCACCTCACCTGCTTCCC
CTTCACCAGCCGCTTCTGCGAGAAGTATGAGCTGGACCAGGTGCTGCAC
NOV16e,
CG52414-03
Protein Sequence SEQ ID NO: 230 827 aa MW at 93378.2 kD
MASADKNGGSVSSVSSSRLQSRKPPNLSITTPPPEKETQAPGEQDSMLPERKNPAYLKSVSLQEPRSR
WQESSEKRPGFRRQASLSQSIRKGAAQWFGVSGDWEGQRQQWQRRSLHHCSMRYGRLAASCQRDLELP
SQEAPSFQGTESPKPCKMPKIVDPLARGRAFRHPEEMDRPHAPHPPLTPGVLSLTSFTSAASGYSHLP
RRKRMSVAHMSLQAAAALLKGRSVLDATGQRCRVVKRSFAFPSFLEEDVNDGADTFDSSFFSKEEMSS
MPDDVFESPPLSASYFRGIPHSASPVSPDCVQIPLKEYGRAPVPGPRRGKRIASKVKHFAFDRKKRHY
GLGVVGNWLNRSYRRSISSTVQRQLESFDSHRPYFTYWLTFAAVIITLLVICTYGIAPVGFAQNVTTQ
LVLRNKGVYESVKYIQQENFWVGPSSIDLIHLGAKFSPCIRKDGQIEQLVLRERDLERDSGCCVQNDH
SGCIQTQRKDCSETLATFVKWQDDTGPPMDKSDLGQKRTSGAVCHQDPRTCEEPASSGAAIWPDDITK
WPICTEQAASNHTGFLHMDCEIKGRPCCIGTKGSCEITTREYCEFMHGYFHEEATLCSQAACLDAACG
LLPFLNPEVPDQFYRLWLSLFLHAGVVHCLVSAAFQMTILRDLEKLACWHRIAIIFILSGITGNLASA
IFLPYAAEVGPAGSQFGLLACLFVELFQSWPLLERPWAAFLNLSAIAAFLFICGLLPWIDNIAAIFGF
LSGLLLAFAFLPYITFGTSDKYRKRALILVSLLAFAGLFAALVLWLYIYPINWPWIEHLTCFPFTSRF
CEKYELDQVLH
NOV16f,
13379509 SNP
for CG52414-02 SEQ ID NO: 231 3040 bp SNP: 873 C/T
DNA Sequence ORF Start: ATG at 338 ORF Stop: TGA at 2819
TTTGGGGCCGCAGGGAGGTTCCCAGACCAGAGGACTGTTGTTAGGTGATTGGCTGTGAACGCCCTGAGGCC
AGTGCCCCTCGCTGCTTGGCACTCGGAGATGCCTGATTAGCACCTTTAATCCCTTACCAATGAGGCACCTG
GAATTGGCCCCATTTTACAGATGGGGAGACTGAGCCACCTGTCTGTCCAGCCACCCTTCCACAGACTGAGG
CTTGACACCGGAGCATCTGTACAGAGCAAGGAGAAGACAAGAACATGCTCTAAAGCCCTTCACAGCAAGAC
CCAGGAAGCCGCGGGCAAACTCAGACTCGAAGCCCTCCCACCTCCTGCCCACAATGGCCTCTGCTGACAAG
AATGGCGGGAGCGTGTCCTCTGTGTCCAGCAGCCGCCTGCAGAGCCGGAAGCCACCCAACCTCTCCATCAC
CATCCCGCCACCCGAGAAAGAGACCCAGGCCCCTGGCGAGCACGACAGCATGCTGCCTGAGAGCAAGAACC
CAGCCTACTTGAAGAGCGTCAGCCTCCAGGAGCCACGCACCCGATGGCACGAGAGTTCAGAGAAGCGCCCT
GGCTTCCGCCGCCAGGCCTCACTGTCCCAGAGCATCCGCAAGGGCGCAGCCCAGTGGTTTGGAGTCAGCGG
CGACTGGGAGGGGCAGCGGCAGCAGTGGCAGCGCCGCAGCCTGCACCACTGCAGCATGCGCTACGGCCGCC
TGAAGGCCTCGTGCCAGCGTGACCTGGAGCTCCCCAGCCAGGAGGCACCGTCCTTCCAGGGCACTGAGTCC
CCAAAGCCCTGCAAGATGCCCAAGATTGTGGATCCGCTGGCCCGGGGCCGGGCCTTCCGCCACCCGGAGGA
GATGGACAGGCCCCACGCCCTGCACCCACCGCTGACCCCCGGAGTCCTGTCCCTCACCTCCTTCACCAGTG
TCCGTTCTGGCTACTCCCACCTGCCACGCCGCAAGAGAATGTCTGTGGCCCACATGAGCTTGCAAGCTGCC
GCTGCCCTCCTCAAGGGGCGCTCGGTGCTGGATGCCACCGGACAGCGGTGCCCGGTGGTCAAGCGCAGCTT
TGCCTTCCCGAGCTTCCTGGAGGAGGATGTGGTCGATGGGGCAGACACGTTTGACTCCTCCTTTTTTAGTA
AGGAAGAAATGAGCTCCATGCCTGATGATGTCTTTGAGTCCCCCCCACTCTCTGCCAGCTACTTCCGAGGG
ATCCCACACTCAGCCTCCCCTGTCTCCCCCGATGGGGTGCAAATCCCTCTGAAGGAGTATGGCCGAGCCCC
AGTCCCCGGGCCCCGGCGCGGCAAGCGCATCGCCTCCAAGGTGAAGCACTTTGCCTTTGATCGGAAGAAGC
GGCACTACGGCCTCGGCGTGGTGGGCAACTGGCTGAACCGCAGCTACCGCCGCAGCATCAGCAGCACTGTG
CAGCGGCAGCTGGAGAGCTTCGACAGCCACCGGCCCTACTTCACCTACTGGCTGACCTTCGTCCATGTCAT
CATCACGCTGCTGGTGATTTGCACCTATGGCATCGCACCCGTGGGCTTTGCCCAGCACGTCACCACCCAGC
TGGTGCTGCGGAACAAAGGTGTGTACGAGAGCGTGAAGTACATCCAGCAGGAGAACTTCTGGGTTGGCCCC
AGCTCGATTGACCTGATCCACCTGGGGGCCAAGTTCTCACCCTGCATCCGGAAGGACGGGCAGATCGACCA
GCTGGTGCTGCGCGAGCGAGACCTGGAGCGGGACTCAGGCTGCTGTGTCCAGAATGACCACTCCGGATGCA
TCCAGACCCAGCGGAAGGACTGCTCGGAGACTTTGGCCACTTTTGTCAAGTGGCAGGATGACACTGGGCCC
CCCATGGACAAGTCTGATCTGGGCCAGAAGCGGACTTCGGGGGCTGTCTGCCACCAGGACCCCAGGACCTG
CGAGGAGCCAGCCTCCAGCGGTGCCCACATCTGGCCCGATGACATCACTAAGTGGCCGATCTGCACAGAGC
AGGCCAGGAGCAACCACACAGGCTTCCTGCACATGGACTGCGAGATCAAGGGCCGCCCCTGCTGCATCGGC
ACCAAGGGCAGCTGTGAGATCACCACCCGGGAATACTGTGAGTTCATGCACGGCTATTTCCATGAGGAAGC
AACACTCTGCTCCCAGGTGCACTGCTTGGACAAGGTGTGTGGGCTGCTGCCCTTCCTCAACCCTGAGGTCC
CAGATCAGTTCTACAGGCTCTGGCTGTCTCTCTTCCTACATGCTGGGGTGGTGCACTGCCTCGTGTCTGTG
GTCTTTCAAATGACCATCCTGAGGGACCTGGAGAAGCTGGCCGGCTGGCACCGTATCGCCATCATCTTCAT
CCTCAGTGGCATCACAGGCAACCTCGCCAGTGCCATCTTTCTCCCATACCGGGCAGAGGTAGGCCCGGCCG
GCTCACAGTTCCGCCTCCTCGCCTGCCTCTTCGTGGAGCTCTTCCAGAGCTGGCCGCTGCTGGAGAGGCCC
TGGAAGCCCTTCCTCAACCTCTCGGCCATCGTGCTCTTCCTGTTCATCTGTGGCCTCCTGCCCTGGATCGA
CAACATCCCCCACATCTTCGCCTTCCTCAGTGGCCTGCTGCTGGCCTTCGCCTTCCTGCCCTACATCACCT
TCGGCACCAGCGACAAGTACCGCAACCGGGCACTCATCCTGGTGTCACTGCTGGCCTTTGCCGGCCTCTTC
GCCGCCCTCGTGCTGTGGCTGTACATCTACCCCATTAACTGGCCCTGGATCGAGCACCTCACCTGCTTCCC
CTTCACCAGCCGCTTCTGCGAGAAGTATGAGCTGGACCAGGTGCTGCACTGACCGCTGGGCCACACGCCTG
CCCCTCAGCCCTGCTGGAACAGGGTCTGCCTGCGAGGCCTGCCCTCTGCAGAGCGCTCTCTGTGTGCCAGA
GAGCCAGAGACCCAACACAGGGCCCGGCCTCTGGACCTGGGTGCCCCCCTGCCAGGCGAGGCTGACTCCGC
GTGAGATAGATGGTTGGTTAAGGCGGGGTTTTTCCGGGCCGCGCCCCCCCCCTCTAAA
NOV16f,
13379509 SNP
for CG52414-02 SNP to Leu
Protein Sequence SEQ ID NO: 232 827 aa at position 179
MASADKNGGSVSSVSSSRLQSRKPPNLSITIPPPEKETQAPGEQDSMLPERKNPAYLKSVSLQEPRSRWQE
SSEKRPGFRRQASLSQSIRKGAAQWFGVSGDWEGQRQQWQRRSLHHCSMRYGRLKASCQRDLELPSQEAPS
FQGTESPKPCKMPKIVDPLARGRAFRHPEEMDRPHALHPPLTPGVLSLTSFTSVRSGYSHLPRRKRMSVAH
MSLQAAAALLKGRSVLDATGQRCRVVKRSFAFPSFLEEDVVDGADTFDSSFFSKEEMSSMPDDVFESPPLS
ASYFRGIPHSASPVSPDGVQIPLKEYGRAPVPGPRRGKRIASKVKHFAFDRKKRHYGLGVVGNWLNRSYRR
SISSTVQRQLESFDSHRPYFTYWLTFVHVIITLLVICTYGIAPVGFAQHVTTQLVLRNKGVYESVKYIQQE
NFWVGPSSIDLIHLGAKFSPCIRKDGQIEQLVLRERDLERDSGCCVQNDHSGCIQTQRKDCSETLATFVKW
QDDTGPPMDKSDLGQKRTSGAVCHQDPRTCEEPASSGAHIWPDDITKWPICTEQARSNHTGFLHMDCEIKG
RPCCIGTKGSCEITTREYCEFMHGYFHEEATLCSQVHCLDKVCGLLPFLNPEVPDQFYRLWLSLFLHAGVV
HCLVSVVFQMTILRDLEKLAGWHRIAIIFILSGITGNLASAIFLPYRAEVGPACSQFGLLACLFVELFQSW
PLLERPWKAFLNLSAIVLFLFICGLLPWIDNIAHIFGFLSGLLLAFAFLPYITFGTSDKYRKRALILVSLL
AFAGLFAALVLWLYIYPINWPWIEHLTCFPFTSRFCEKYELDQVLH
NOV16g,
13381817 SNP
for CG52414-02 SEQ ID NO: 233 3040 bp SNP: 971 G/A
DNA Sequence ORF Start: ATG at 338 ORF Stop: TGA at 2819
TTTGGGGCCGCAGGGAGGTTCCCAGACCAGACCACTGTTGTTAGGTGATTGGCTGTGAACGCCCTGAGGCC
AGTGCCCCTCGCTGCTTGGCACTCGGAGATGCCTGATTAGCACCTTTAATCCCTTACCAATGAGGCAGGTG
GAATTGGCCCCATTTTACAGATGGGGAGACTGAGCCACCTGTCTGTCCAGCCACCCTTCCACAGACTGAGG
CTTGACACCGGAGCATCTGTACAGAGCAAGGAGAAGACAAGAACATGCTCTAAAGCCCTTCACAGCAAGAC
CCAGGAAGCCGCGGGCAAACTCAGACTCGAAGCCCTCCCACCTCCTGCCCACAATGGCCTCTGCTGACAAG
AATGGCGCGAGCCTGTCCTCTGTGTCCAGCAGCCGCCTGCAGAGCCGGAAGCCACCCAACCTCTCCATCAC
CATCCCGCCACCCGAGAAAGAGACCCAGGCCCCTGGCGAGCAGGACAGCATGCTGCCTGAGACGAAGAACC
CAGCCTACTTGAAGAGCGTCAGCCTCCAGGAGCCACGCAGCCGATGGCAGGAGAGTTCAGAGAAGCGCCCT
GGCTTCCGCCGCCAGGCCTCACTGTCCCAGAGCATCCGCAAGGGCGCAGCCCAGTGCTTTGGAGTCAGCGG
CGACTGGGAGGGGCAGCGGCAGCAGTGGCAGCGCCGCAGCCTGCACCACTGCAGCATGCGCTACGGCCGCC
TGAAGGCCTCGTGCCAGCGTGACCTGGACCTCCCCAGCCACGAGGCACCGTCCTTCCAGGGCACTGAGTCC
CCAAAGCCCTGCAAGATGCCCAAGATTGTGGATCCGCTGGCCCGGGCCCCGCCCTTCCGCCACCCGGAGGA
GATGGACAGGCCCCACGCCCCGCACCCACCGCTGACCCCCGGAGTCCTGTCCCTCACCTCCTTCACCAGTG
TCCGTTCTGGCTACTCCCACCTGCCACGCCGCAAGAGAATGTCTCTGACCCACATGAGCTTGCAAGCTGCC
GCTGCCCTCCTCAAGGGGCGCTCGGTGCTGGATCCCACCGGACAGCGGTGCCGGGTGGTCAAGCGCAGCTT
TGCCTTCCCGAGCTTCCTGGAGGAGGATGTGGTCGATGGGCCACACACGTTTGACTCCTCCTTTTTTAGTA
AGGAAGAAATGAGCTCCATGCCTGATGATGTCTTTGAGTCCCCCCCACTCTCTGCCAGCTACTTCCGAGGG
ATCCCACACTCAGCCTCCCCTGTCTCCCCCGATGGGCTCCAAATCCCTCTGAAGGAGTATGGCCGAGCCCC
AGTCCCCGGGCCCCGCCGCGGCAAGCGCATCGCCTCCAAGGTGAAGCACTTTGCCTTTGATCGGAAGAAGC
GGCACTACGGCCTCGGCGTGGTGGGCAACTGGCTGAACCGCAGCTACCGCCGCAGCATCAGCAGCACTGTG
CAGCGGCAGCTGGAGAGCTTCGACAGCCACCGGCCCTACTTCACCTACTGGCTGACCTTCGTCCATGTCAT
CATCACGCTGCTGGTGATTTGCACGTATGGCATCGCACCCGTGGGCTTTGCCCAGCACGTCACCACCCAGC
TGGTGCTGCGGAACAAAGGTGTGTACGAGAGCGTGAAGTACATCCAGCAGGAGAACTTCTGGGTTGGCCCC
AGCTCGATTGACCTGATCCACCTGGGGGCCAAGTTCTCACCCTGCATCCGGAAGGACGGGCACATCGAGCA
GCTGGTGCTGCGCGAGCGAGACCTGGAGCGGGACTCAGGCTGCTGTGTCCAGAATGACCACTCCGGATGCA
TCCAGACCCAGCGGAAGGACTGCTCGGAGACTTTGGCCACTTTTGTCAAGTGGCAGGATGACACTGGGCCC
CCCATGGACAAGTCTCATCTGGGCCAGAAGCGGACTTCGGGGGCTGTCTGCCACCAGGACCCCAGGACCTC
CGAGCAGCCAGCCTCCAGCGGTGCCCACATCTGGCCCGATGACATCACTAAGTGGCCGATCTGCACAGAGC
AGGCCAGGAGCAACCACACAGGCTTCCTGCACATGGACTGCGAGATCAAGGGCCGCCCCTGCTGCATCGGC
ACCAAGGGCAGCTGTGAGATCACCACCCGGGAATACTGTGAGTTCATCCACGGCTATTTCCATGAGGAAGC
AACACTCTGCTCCCAGGTGCACTCCTTGGACAACGTGTGTGGGCTGCTGCCCTTCCTCAACCCTGAGGTCC
CAGATCAGTTCTACAGGCTCTGGCTGTCTCTCTTCCTACATGCTGGGGTGGTGCACTGCCTCGTGTCTGTG
GTCTTTCAAATGACCATCCTGAGGGACCTGGAGAAGCTGGCCGGCTGGCACCGTATCGCCATCATCTTCAT
CCTCAGTGGCATCACAGGCAACCTCGCCAGTGCCATCTTTCTCCCATACCGGGCAGAGGTAGGCCCGGCCG
GCTCACAGTTCGGCCTCCTCGCCTGCCTCTTCGTGGAGCTCTTCCAGAGCTGGCCGCTGCTGGAGAGGCCC
TCGAAGGCCTTCCTCAACCTCTCGGCCATCGTGCTCTTCCTGTTCATCTGTGGCCTCCTGCCCTGGATCGA
CAACATCGCCCACATCTTCGGCTTCCTCAGTGGCCTGCTGCTGGCCTTCGCCTTCCTGCCCTACATCACCT
TCGGCACCAGCGACAAGTACCGCAAGCGGGCACTCATCCTGGTGTCACTGCTGGCCTTTGCCGGCCTCTTC
GCCGCCCTCGTGCTGTGGCTGTACATCTACCCCATTAACTGGCCCTGGATCGAGCACCTCACCTGCTTCCC
CTTCACCAGCCGCTTCTGCGAGAAGTATGAGCTGGACCAGGTGCTGCACTGACCGCTGGGCCACACGGCTG
CCCCTCAGCCCTGCTGGAACAGGGTCTGCCTGCGAGGGCTGCCCTCTGCAGAGCGCTCTCTGTGTGCCAGA
GAGCCAGAGACCCAAGACAGGGCCCGGGCTCTGGACCTGGGTGCCCCCCTGCCAGGCGAGGCTGACTCCGC
GTGAGATAGATGGTTGGTTAAGGCGGGGTTTTTCCGGGCCGCGCCCCCCCCCTCTAA
NOV16g,
13381817 SNP
for CG52414-02 SNP: Ala to Thr
Protein Sequence SEQ ID NO: 234 827 aa at position 212
MASADKNGGSVSSVSSSRLQSRKPPNLSITIPPPEKETQAPGEQDSMLPERKNPAYLKSVSLQEPRSRWQE
SSEKRPGFRRQASLSQSIRKGAAQWFGVSGDWEGQRQQWQRRSLHHCSMRYGRLAASCQRDLELPSQEAPS
FQGTESPKPCKMPKIVDPLARGRAFRHPEEMDRPHAPHPPLTPGVLSLTSFTSVRSGYSHLPRRKRMSVTH
MSLQAAAALLKGRSVLDATGQRCRVVKRSFAFPSFLEEDAADGAATFDSSFFSKEEMSSMPDDVFESPPLS
ASYFRGIPHSASPVSPDGVQIPLKEYGRAPVPGPRRGKRIASKVKHFAFDRKKRHYGLGVVGNWLNRSYRR
SISSTVQRQLESFDSHRPYFTYWLTFVHVIITLLVICTYGIAPVGFAQHVTTQLVLRNKGAAESVKYIQQE
NFWVGPSSIDLIHLGAKFSPCIRKDGQIEQLVLRERDLERDSGCCVQNDHSGCIQTQRKDCSETAATFVKW
QDDTGPPMDKSDLGQKRTSGAVCHQDPRTCEEPASSGAAIWPDDITKWPICTEQARSNHTGFLHMDCEIKG
RPCCIGTKGSCEITTREYCEFMHGYFHEEATLCSQVHCLDKVCGLLPFLNPEVPDQFYRLWLSLFLHAGAA
HCLVSVVFQMTILRDLEKLAGWHRIAIIFILSGITGNLASAIFLPYRAEVGPAGSQFGLLACLFVELFQSW
PLLERPWKAFLNLSAIVLFLFICGLLPWIDNIAHIFGFLSGLLLAFAFLPYITFGTSDKYRKRALILVSLL
AFAGLFAALVLWLYIYPINWPWIEHLTCFPFTSRFCEKYELDQVLH
NOV16h,
13382069 SNP
for CG52414-02 SEQ ID NO: 235 3040 bp SNP: 1247 C/T
DNA Sequence ORF Start: ATG at 338 ORF Stop: TAA at 1247
TTTGGGGCCGCAGGGAGGTTCCCAGACCAGAGGACTGTTGTTAGGTGATTGGCTGTGAACGCCCTGAGGCC
AGTGCCCCTCGCTGCTTGGCACTCGGAGATGCCTGATTAGCACCTTTAATCCCTTACCAATGAGGCAGGTG
GAATTGGCCCCATTTTACAGATGGGGAGACTGAGCCACCTGTCTGTCCAGCCACCCTTCCACAGACTGAGG
CTTGACACCGGAGCATCTGTACAGAGCAAGGAGAAGACAAGAACATGCTCTAAAGCCCTTCACAGCAAGAC
CCAGGAAGCCGCGGGCAAACTCAGACTCGAAGCCCTCCCACCTCCTGCCCACAATGGCCTCTGCTGACAAG
AATGGCGGGAGCGTGTCCTCTGTGTCCAGCAGCCGCCTGCAGAGCCGGAAGCCACCCAACCTCTCCATCAC
CATCCCGCCACCCGAGAAAGAGACCCAGGCCCCTGGCGAGCAGGACAGCATGCTGCCTGAGAGGAAGAACC
CAGCCTACTTGAAGAGCGTCAGCCTCCAGGAGCCACGCAGCCGATGGCAGGAGAGTTCAGAGAAGCGCCCT
GGCTTCCGCCGCCAGGCCTCACTGTCCCAGAGCATCCGCAAGGGCGCAGCCCAGTGGTTTGGAGTCAGCGG
CGACTGGGAGGGGCAGCGGCAGCAGTGGCAGCGCCGCAGCCTGCACCACTGCAGCATGCGCTACGGCCGCC
TCAAGGCCTCGTGCCAGCGTGACCTGGAGCTCCCCAGCCAGGAGGCACCGTCCTTCCAGGGCACTGAGTCC
CCAAAGCCCTGCAAGATGCCCAAGATTGTGGATCCGCTGGCCCGGGGCCGGGCCTTCCGCCACCCGGAGGA
CATGGACAGCCCCCACGCCCCGCACCCACCGCTGACCCCCGGAGTCCTGTCCCTCACCTCCTTCACCAGTG
TCCGTTCTGGCTACTCCCACCTGCCACGCCGCAAGAGAATGTCTGTGGCCCACATGAGCTTGCAAGCTGCC
GCTGCCCTCCTCAAGGGGCGCTCGGTGCTGGATGCCACCGGACAGCGGTGCCGGGTGGTCAAGCGCAGCTT
TGCCTTCCCGAGCTTCCTGGAGGAGGATGTGGTCGATGGGGCAGACACGTTTGACTCCTCCTTTTTTAGTA
AGGAAGAAATGAGCTCCATGCCTGATGATGTCTTTGAGTCCCCCCCACTCTCTGCCAGCTACTTCCGAGGG
ATCCCACACTCAGCCTCCCCTGTCTCCCCCGATGGGGTGTAAATCCCTCTGAAGGAGTATGGCCGAGCCCC
AGTCCCCGGGCCCCGGCGCGGCAAGCGCATCGCCTCCAAGGTGAAGCACTTTGCCTTTGATCGGAAGAAGC
GGCACTACGGCCTCGGCGTGGTGGGCAACTGGCTGAACCGCAGCTACCGCCGCAGCATCAGCAGCACTGTG
CAGCGGCAGCTGGAGAGCTTCGACAGCCACCGGCCCTACTTCACCTACTGGCTGACCTTCGTCCATGTCAT
CATCACGCTCCTGGTGATTTGCACGTATGGCATCGCACCCGTGGGCTTTGCCCAGCACGTCACCACCCAGC
TGGTGCTGCGGAACAAAGGTGTGTACGAGAGCGTGAAGTACATCCAGCAGGAGAACTTCTGGGTTGGCCCC
AGCTCGATTGACCTGATCCACCTGGGGGCCAAGTTCTCACCCTGCATCCGGAAGGACGGGCAGATCGAGCA
GCTGGTGCTGCGCCACCGAGACCTGGAGCGGGACTCAGGCTGCTGTGTCCAGAATGACCACTCCGGATGCA
TCCAGACCCAGCGGAAGGACTGCTCGGAGACTTTGGCCACTTTTGTCAAGTGGCAGGATGACACTGGGCCC
CCCATGGACAAGTCTCATCTGGGCCAGAAGCGGACTTCGGGGGCTGTCTGCCACCAGGACCCCAGGACCTG
CGAGGAGCCAGCCTCCAGCGGTCCCCACATCTGGCCCGATGACATCACTAAGTGGCCGATCTGCACAGAGC
AGGCCAGGAGCAACCACACAGGCTTCCTGCACATGGACTCCGAGATCAAGGGCCGCCCCTGCTGCATCGGC
ACCAAGGGCAGCTGTGAGATCACCACCCGGGAATACTGTGAGTTCATGCACGGCTATTTCCATCAGGAAGC
AACACTCTGCTCCCAGGTGCACTGCTTGGACAAGGTGTGTGGGCTGCTGCCCTTCCTCAACCCTGAGGTCC
CAGATCAGTTCTACAGGCTCTGGCTGTCTCTCTTCCTACATGCTGGGGTGGTGCACTGCCTCGTGTCTGTG
GTCTTTCAAATGACCATCCTGAGGGACCTCGAGAAGCTGGCCGGCTGGCACCGTATCGCCATCATCTTCAT
CCTCAGTGGCATCACAGGCAACCTCGCCAGTGCCATCTTTCTCCCATACCGGGCAGAGGTAGGCCCGGCCG
GCTCACAGTTCGGCCTCCTCGCCTCCCTCTTCGTGGAGCTCTTCCAGAGCTGGCCGCTGCTGGACAGGCCC
TCGAAGGCCTTCCTCAACCTCTCGGCCATCGTGCTCTTCCTGTTCATCTGTGGCCTCCTGCCCTCGATCGA
CAACATCGCCCACATCTTCGGCTTCCTCAGTGGCCTGCTGCTGGCCTTCGCCTTCCTGCCCTACATCACCT
TCGGCACCAGCGACAAGTACCGCAAGCGGGCACTCATCCTGGTGTCACTGCTGGCCTTTGCCGGCCTCTTC
GCCGCCCTCGTGCTGTGGCTGTACATCTACCCCATTAACTGGCCCTGGATCGAGCACCTCACCTGCTTCCC
CTTCACCAGCCGCTTCTGCCAGAAGTATGAGCTGGACCAGGTGCTGCACTCACCGCTGGGCCACACGGCTG
CCCCTCAGCCCTGCTGGAACAGGGTCTGCCTGCGAGGGCTGCCCTCTGCAGAGCGCTCTCTGTGTGCCAGA
GAGCCAGAGACCCAAGACAGGGCCCGGGCTCTGGACCTGGGTGCCCCCCTGCCAGGCGAGGCTGACTCCGC
GTGAGATAGATCGTTGGTTAAGGCGGGGTTTTTCCGGGCCGCGCCCCCCCCCTCTAAA
NOV16h,
13382069 SNP
for CG52414-02 SNP: Gln to STOP
Protein Sequence SEQ ID NO: 236 827 aa at position 304
MASADKNGGSVSSVSSSRLQSRKPPNLSITIPPPEKETQAPGEQDSMLPERKNPAYLKSVSLQEPRSRWQE
SSEKRPGFRRQASLSQSIRKGAAQWFGVSGDWEGQRQQWQRRSLHHCSMRYGRLKASCQRDLELPSQEAPS
FQGTESPKPCKMPKIVDPLARGRAFRHPEEMDRPHAPHPPLTPGVLSLTSFTSVRSGYSHLPRRKRNSVAH
MSLQAAAALLKGRSVLDATGQRCRVVKRSFAFPSFLEEDVVDGADTFDSSFFSKEEMSSMPDDVFESPPLS
ASYFRGIPHSASPVSPDGV*
NOV16i,
13382069 SNP
for CG52414-02 SEQ ID NO: 237 3040 bp SNP: 2493 C/T
DNA Sequence ORF Start: ATG at 338 ORF Stop: TGA at 2819
TTTGGGGCCGCAGGGAGGTTCCCAGACCAGAGGACTGTTGTTAGUTGATTGGCTGTGAACGCCCTGAGGCC
AGTGCCCCTCGCTGCTTGCCACTCGGAGATGCCTGATTACCACCTTTAATCCCTTACCAATCAGGCAGGTG
GAATTGGCCCCATTTTACAGATGGGGAGACTGAGCCACCTGTCTGTCCAGCCACCCTTCCACAGACTGAGG
CTTGACACCGGAGCATCTGTACAGAGCAAGGAGAAGACAAGAACATGCTCTAAAGCCCTTCACAGCAAGAC
CCAGGAAGCCGCGGGCAAACTCAGACTCGAAGCCCTCCCACCTCCTGCCCACAATGGCCTCTGCTGACAAG
AATGGCGGGAGCGTGTCCTCTGTGTCCAGCAGCCGCCTGCAGAGCCGGAAGCCACCCAACCTCTCCATCAC
CATCCCGCCACCCGAGAAAGAGACCCAGGCCCCTGGCGAGCAGGACAGCATGCTGCCTGAGAGGAAGAACC
CAGCCTACTTGAAGAGCGTCAGCCTCCAGCAGCCACGCAGCCGATGGCAGGACAGTTCAGAGAAGCGCCCT
GGCTTCCGCCGCCAGGCCTCACTGTCCCACAGCATCCGCAAGGGCGCAGCCCAGTGGTTTGGAGTCAGCGG
CGACTCGGAGGGGCAGCGGCAGCAGTGGCAGCGCCGCAGCCTGCACCACTGCAGCATGCGCTACGGCCGCC
TGAAGGCCTCGTGCCAGCGTGACCTGGAGCTCCCCAGCCAGGAGGCACCGTCCTTCCAGGGCACTGAGTCC
CCAAAGCCCTGCAAGATGCCCAAGATTGTGGATCCGCTGGCCCGGGGCCGGGCCTTCCGCCACCCGGAGGA
GATGGACAGGCCCCACCCCCCGCACCCACCGCTGACCCCCGGAGTCCTGTCCCTCACCTCCTTCACCAGTG
TCCGTTCTGGCTACTCCCACCTGCCACGCCGCAAGACAATGTCTGTGGCCCACATCAGCTTGCAAGCTGCC
GCTGCCCTCCTCAAGGGGCGCTCGGTGCTGGATGCCACCGGACAGCGGTGCCGGGTGGTCAAGCGCAGCTT
TGCCTTCCCGAGCTTCCTGGAGGAGGATGTGGTCGATGGGGCAGACACGTTTGACTCCTCCTTTTTTAGTA
AGGAAGAAATGAGCTCCATGCCTGATGATGTCTTTGAGTCCCCCCCACTCTCTGCCAGCTACTTCCGAGGG
ATCCCACACTCAGCCTCCCCTGTCTCCCCCGATGGGGTGCAAATCCCTCTGAAGGAGTATGGCCGAGCCCC
AGTCCCCGGGCCCCGGCGCGGCAAGCGCATCGCCTCCAAGGTGAAGCACTTTGCCTTTGATCGGAAGAAGC
GGCACTACGGCCTCGGCGTGGTGGGCAACTGGCTGAACCGCAGCTACCGCCGCAGCATCAGCAGCACTGTG
CAGCGGCAGCTGGAGAGCTTCGACAGCCACCGGCCCTACTTCACCTACTGGCTGACCTTCGTCCATGTCAT
CATCACGCTGCTGGTGATTTGCACGTATGGCATCGCACCCGTGGGCTTTGCCCAGCACGTCACCACCCAGC
TGGTGCTGCGGAACAAAGGTGTGTACGAGAGCGTGAAGTACATCCAGCAGGAGAACTTCTGGGTTGGCCCC
AGCTCGATTCACCTUATCCACCTGGGGGCCAAGTTCTCACCCTGCATCCGGAAGGACGGGCAGATCGACCA
GCTGGTGCTGCGCGAGCGAGACCTGGAGCGGGACTCAGGCTGCTGTGTCCAGAATGACCACTCCGGATGCA
TCCAGACCCAGCGGAAGGACTGCTCGGAGACTTTGGCCACTTTTGTCAAGTGGCAGGATGACACTGGGCCC
CCCATGGACAAGTCTGATCTGGGCCAGAAGCGGACTTCGGGGGCTGTCTGCCACCAGGACCCCAGGACCTG
CGAGGAGCCAGCCTCCAGCGGTGCCCACATCTGGCCCGATGACATCACTAAGTGGCCGATCTGCACAGAGC
AGGCCAGGAGCAACCACACAGGCTTCCTGCACATGGACTGCGAGATCAAGCCCCGCCCCTGCTGCATCGGC
ACCAAGGGCAGCTGTGAGATCACCACCCGGGAATACTGTGAGTTCATGCACGGCTATTTCCATGAGGAAGC
AACACTCTGCTCCCAGGTGCACTGCTTGGACAAGGTGTGTGGGCTGCTGCCCTTCCTCAACCCTGAGGTCC
CAGATCAGTTCTACAGGCTCTGGCTGTCTCTCTTCCTACATGCTGGGGTGGTGCACTGCCTCGTGTCTGTG
GTCTTTCAAATGACCATCCTGAGGGACCTGGAGAAGCTGGCCGGCTGGCACCGTATCGCCATCATCTTCAT
CCTCAGTGGCATCACAGCCAACCTCGCCAGTGCCATCTTTCTcCCATACCGCGCAGAGGTAGGCCCCGCCG
GCTCACAGTTCGCCCTCCTCGCCTGCCTCTTCGTGGAGCTCTTCCAGACCTGGCCGCTGCTGGAGAGGCCC
TGGAACGTCTTCCTCAACCTCTCGCCCATCGTCCTCTTCCTGTTCATCTGTGGCCTCCTGCCCTGGATCGA
CAACATCGCCCACATCTTCGGCTTCCTCAGTGGCCTGCTGCTGGCCTTCGCCTTCCTGCCCTACATCACCT
TCGGCACCAGCGACAAGTACCGCAAGCGGGCACTCATCCTGGTGTCACTGCTGGCCTTTGCCGGCCTCTTC
GCCGCCCTCGTCCTGTGGCTGTACATCTACCCCATTAACTGGCCCTGGATCGAGCACCTCACCTGCTTCCC
CTTCACCAGCCGCTTCTGCGAGAAGTATGAGCTGGACCAGGTGCTGCACTGACCGCTGGGCCACACGGCTG
CCCCTCAGCCCTGCTGGAACAGGGTCTGCCTGCGAGGGCTCCCCTCTGCAGAGCGCTCTCTGTGTGCCAGA
GAGCCAGAGACCCAAGACAGGGCCCGGGCTCTGGACCTGGGTGCCCCCCTGCCACGCGAGCCTGACTCCGC
GTGAGATAGATGGTTGGTTAAGGCGGGGTTTTTCCGGGCCGCGCCCCCCCCCTCTAAA
NOV16i,
13381560 SNP
for CG52414-02 SNP: Ala to Val
Protein Sequence SEQ ID NO: 238 827 aa at position 719
MASADKNGGSVSSVSSSRLQSRKPPNLSITIPPPEKETQAPGEQDSMLPERKNPAYLKSVSLQEPRSRWQE
SSEKRPGFRRQASLSQSIRKGAAQWFGVSGDWEGQRQQWQRRSLHHCSMRYGRLKASCQRDLELPSQEAPS
FQGTESPKPCKMPKIVDPLARGRAFRHPEEMDRPHAPHPPLTPGVLSLTSFTSVRSGYSHLPRRKRMSVAH
MSLQAAAALLKCRSVLDATGQRCRVVKRSFAFPSFLEEDVVDGADTFDSSFFSKEEMSSMPDDVFESPPLS
ASYFRGIPHSASPVSPDGVQIPLKEYGRAPVPGPRRGKRIASKVKHFAFDRKKRHYGLGVVGNWLNRSYRR
SISSTVQRQLESFDSHRPYFTYWLTFVHVIITLLVICTYGIAPVGFAQHVTTQLVLRNKGVYESVKYIQQE
NFWVGPSSIDLIHLGAKFSPCTRKDGQTEQLVLRERDLERDSGCCVQNDHSGCIQTQRKDCSETLATFVKW
QDDTGPPMDKSDLGQKRTSGAVCHQDPRTCEEPASSGAHIWPDDITKWPICTEQARSNHTGFLHMDCEIKG
RPCCIGTKGSCEITTREYCEFMHGYFHEEATLCSQVHCLDKVCGLLPFLNPEVPDQFYRLWLSLFLHAGVV
HCLVSVVFQMTILRDLEKLAGWHRIAIIFILSGITGNLASAIFLPYRAEVGPAGSQFGLLACLFVELFQSW
PLLERPWKVFLNLSAIVLFLFICGLLPWIDNIAHIFGFLSGLLLAFAFLPYITFGTSDKYRKRALILVSLL
AFAGLFAALVLWLYIYPINWPWIEHLTCFPFTSRFCEKYELDQVLH

A ClustalW comparison of the above protein sequences yields the following sequence alignment shown in Table 16B.

TABLE 16B
Comparison of the NOV16 protein sequences.

| | |
|---|---|
| NOV16a | -----MASADKNGGSVSSVSSSRLQSRKPPNLSITIPPPEKETQAPGEQDSMLPERKNPA |
| NOV16b | ------------------------------------------------------------ |
| NOV16c | TRSPTMASADKNGGSVSSVSSSRLQSRKPPNLSITIPPPEKETQAPGEQDSMLPERKNPA |
| NOV16d | ------------------------------------------------------------ |
| NOV16e | -----MASADKNGGSVSSVSSSRLQSRKPPNLSITIPPPEKETQAPGEQDSMLPERKNPA |
| NOV16a | YLKSVSLQEPRSRWQESSEKRPGFRRQASLSQSIRKGAAQWFGVSGDWEGQRQQWQRRSL |
| NOV16b | ------------------------------------------------------------ |
| NOV16c | YLKSVSLQEPRSRWQESSEKRPGFRRQASLSQSIRKGAAQWFGVSGDWEGQRQQWQRRSL |
| NOV16d | ------------------------------------------------------------ |
| NOV16e | YLKSVSLQEPRSRWQESSEKRPGFRRQASLSQSIRKGAAQWFGVSGDWEGQRQQWQRRSL |
| NOV16a | HHCSMRYCRLKASCQRDLELPSQEAPSFQGTESPKPCKMPKIVDPLARGRAFRHPEEMDR |
| NOV16b | ------------------------------------------------------------ |
| NOV1Gc | HHCSMRYCRLKASCQRDLELPSQEAPSFQGTESPKPCKMPKIVDPLARGRAFRHPEEMDR |
| NOV16d | ----MRYGRLKASCQRDLELPSQEAPSFQGTESPKPCKMPKIVDPLARGRAFRHPEEMDR |
| NOV16e | HHCSMRYCRLKASCQRDLELPSQEAPSFQGTESPKPCKMPKIVDPLARGRAFRHPEEMDR |
| NOV16a | PHAPHPPLTPCVLSLTSFTSVRSGYSHLPRRKRMSVAHMSLQAAAALLKGRSVLDATGQR |
| NOV16b | ------------------------------------------------------------ |
| NOV16c | PHAPHPPLTPCVLSLTSFTSVRSGYSHLPRRKRMSVAHMSLQAAAALLKGRSVLDATGQR |
| NOV16d | PHAPHPPLTPCVLSLTSFTSVRSGYSHLPRRKRMSVAHMSLQAAAALLKGRSVLDATGQR |
| NOV16e | PHAPHPPLTPCVLSLTSFTSVRSGYSHLPRRKRMSVAHMSLQAAAALLKGRSVLDATGQR |
| NOV16a | CRVVKRSFAFPSFLEEDVVDGADTFDSSFFSKEEMSSMPDDVFESPPLSASYFRGIPNSA |
| NOV16b | ------------------------------------------------------------ |
| NOV16c | CRMVKRSFAFPSFLEEDVVDGADTFDSSFFSKEEMSSMPDDVFESPPLSASYFRGIPHSA |
| NOV16d | CRVVKRSFAFPSFLEEDVVDGADTFDSSFFSKEEMSSMPDDVFESPPLSASYFRGIPHSA |
| NOV16e | CRVVKRSFAFPSFLEEDVVDGADTFDSSFFSKEEMSSMPDDVFESPPLSASYFRGIPHSA |
| NOV16a | SPVSPOGVQIPLKEYGRAPVPGPRRGKRIASKVKHFAFDRKKRHYGLGVVGNWLNRSYRR |
| NOV16b | ------------------------------------------------------------ |
| NOV16c | SPVSPDGVQIPLKEYGRAPVPGPRRGERIASKVKHFAFDRKKRHYGLGVVGNWLNRSYRR |
| NOV16d | SPVSPDGVQIPLKEYGRAPVPGPRRGKRIASKVKHFAFDRKKRNYGLGVVGNWLNRSYRR |
| NOV16e | SFVSPDGVQIPLKEYGRAPVPGPRRGKRIASKVKNFAFDRKKRHYGLGVVGNWLNRSYRR |
| NOV16a | SISSTVQRQLESFDSHRPYFTYWLTFVHVIITLLVICTYGIAPVGFAQHAATQLVLRNKG |
| NOV16b | --------------------------------------------TRSQHVTTQLVLRNKG |
| NOV16c | SISSTVQRQLESFDSNRPYFTYWLTFVHVIITLLVICTYGIAPVGFAQHAATQLVLRNKG |
| NOV16d | SISSTVQRQLESFDSHRPYFTYWLTFVHVIITLLVICTYGIAPVGFAQNVTTQLVLRNKG |
| NOV16e | SISSTVQRQLESFDSHRPYFTYWLTFVHVIITLLVICTYGIAPVGFAQHAATQLVLRNKG |
| NOV16a | VYESVKYIQQENFWVGPSSIDLIHLGAKFSPCIRKDGQIEQLVLRERDLERDSGCCVQND |
| NOV16b | VYESVKYIQQENFWVGPSSIDLIHLGAKFSPCIRKDGQIEQLVLRERDLERDSGCCVQND |
| NOV16c | VYESVKYIQQENFWVGPSSIDLIHLGAKFSPCIRKDGQIEQLVLREROLERDSGCCVQND |
| NOV16d | VYESVKYIQQENFWVGPSSIDLIHLOAKFSPCIRKDGQIEQLVLRERDLERDSGCCVQND |
| NOV16e | VYESVKYIQQENFWVGPSSIDLIHLGAKFSPCIRKDGQIEQLVLRERDLERDSGCCVQND |
| NOV16a | HSGCIQTQRKDCSETLATFVKWQDDTGPFMDKSDLGQKRTSGAVCHQDPRTCEEPASSGA |
| NOV16b | HSSCIQTQRKDCSETLATFVKWQDDTGPPMDKSDLGQKRTSGAVCHQDPRTCEEPASSGA |
| NOV16c | HSGCIQTQRKDCSETLATFVKWQODTGPPMDKSDLGQKRTSGAVCNQOPRTCEEPASSGA |
| NOV16d | HSGCIQTQRKDCSETLATFVKWQDDTGPPMDKSDLGQKRTSGAVCHQDPRTCEEPASSGA |
| NOV16e | HSGCIQTQRKDCSETLATFVKWQDDTGPPMDKSDLGQKRTSGAVCHQDPRTCEEPASSGA |
| NOV16a | HIWPDDITKWFICTEQARSNHTGFLHMDCEIKGRPCCIGTKGSCEITTREYCEFMHCYFH |
| NOV16b | HIWPDDITKWPICTEQARSNHTGFLHMDCEIKGRPCCIGTKGSCEITTREYCEFMHGYFH |
| NOV16c | HIWPDDITKWPICTEQARSNHTGFLHMDCEIKGRPCCIGTKGSCEITTREYCEFMHGYFH |
| NOV16d | HIWPDDITKWPICTEQARSNHTGFLHMDCEIKCRPCCIGTKGSCEITTREYCEFMHGYFH |
| NOV16e | HIWPDDITKWPICTEQARSNHTCFLHMDCEIKGRPCCIGTKGSCEITTREYCEFMHGYFH |
| NOV16a | EEATLCSQVHCLDKVCGLLPFLNPEVPDQFYRLWLSLFLHAGVVHCLVSVVFQMTILRDL |
| NOV16b | EEATLCSQVHCLDKVCGLLPFLNPEVPDQFYRLEG------------------------- |
| NOV16c | EEATLCSQVHCLDKVCGLLPFLNPEVPDQFYRLWLSLFLHAGVVHCLVSVVFQMTILRDL |
| NOV16d | EEATLCSQVHCLDKVCGLLPFLNPEVPDQFYRLWLSLFLHAGVVHCLVSVVFQMTILRDL |
| NOV16e | EEATLCSQVHCLDKVCGLLPFLNPEVPDQFYRLWLSLFLHAGVVHCLVSVVFQMTILRDL |
| NOV16a | EKLAGWHRIAIIFILSGITCNLASAIFLFYRAEVGPAGSQFGLLACLFVELFQSWPLLER |
| NOV16b | ------------------------------------------------------------ |
| NOV16c | EKLAGWHRIAIIFILSGITGNLASAIFLPYRAEVGPACSQFGLLACLFVELFQSWPLLER |
| NOV16d | EKLACWHRIAIIFILSGITGNLASTIFLPYRAEVGPAGSQFGLLACLFVELFQSWPLLER |
| NOV16e | EKLACWHRIAIIFILSGITGNLASAIFLPYRAEVGPAGSQFGLLACLFVELFQSWPLLER |
| NOV16a | PWKAFLNLSAIVLFLFICGLLPWIDNIAHIFGFLSGLLLAFAFLPYITFGTSDKYRKRAL |
| NOV16b | ------------------------------------------------------------ |
| NOV16c | PWKAFLNLSAIVLFLFICCLLPWIDNIAHIFGFLSGLLLAFAFLPYITFGTSDKYRKRAL |
| NOV16d | PWKAFLNLSTIVLFLFICGLLPWIDNIAHIFGFLSGLLLAFAFLPYITFGTSDKYRKRAL |
| NOV16e | PWKAFLNLSAIVLFLFICGLLPWIDNIAHIFGFLSGLLLAFAFLPYITFGTSDKYRKRAL |
| NOV16a | ILVSLLAFAGLFAALVLWLYIYPINWPWIEHLTCFPFTSRFCEKYELDQVLH--- |
| NOV16b | ------------------------------------------------------- |
| NOV16c | ILVSLLAFAGLFAALVLWLYIYPINWPWIEHLTCFPFTSRFCEKYELDQVLHLEG |
| NOV16d | ILVSLLAFACLFAALVLWLYIYPINWPWIEHLTCFPFTSRFCEKYELDQVLH--- |
| NOV16e | ILVSLLAFAGLFAALVLWLYIYPINWPWIEHLTCFPFTSRFCEKYELDQVLH--- |
| NOV16a | (SEQ ID NO: 222) |
| NOV16b | (SEQ ID NO: 224) |
| NOV16c | (SEQ ID NO: 226) |
| NOV16d | (SEQ ID NO: 228) |
| NOV16e | (SEQ ID NO: 230) |

Further analysis of the NOV16a protein yielded the following properties shown in Table 16C.

TABLE 16C
Protein Sequence Properties NOV16a

| SignalP | No Known Signal Sequence Predicted | | | | |
|---|---|---|---|---|---|
| analysis: | | | | | |
| PSORT II | PSG: a new signal peptide prediction method | | | | |
| analysis: | N-region: length 6; pos. chg 1; neg. chg 1 | | | | |
| | H-region: length 11; peak value 5.03 | | | | |
| | PSG score: 0.62 | | | | |
| | GvH: von Heijne's method for signal seq. recognition | | | | |
| | GvH score (threshold: −2.1): −7.64 | | | | |
| | possible cleavage site: between 21 and 22 | | | | |
| | >>> Seems to have no N-terminal signal peptide | | | | |
| | ALOM: Klein et al's method for TM region allocation | | | | |
| | Init position for calculation: 1 | | | | |
| | Tentative number of TMS(s) for the threshold 0.5: 7 | | | | |
| | INTEGRAL | Likelihood = | −6.42 | Transmembrane | 381-397 |
| | INTEGRAL | Likelihood = | −4.25 | Transmembrane | 630-646 |
| | INTEGRAL | Likelihood = | −3.08 | Transmembrane | 666-682 |
| | INTEGRAL | Likelihood = | 0.37 | Transmembrane | 697-713 |
| | INTEGRAL | Likelihood = | −9.08 | Transmembrane | 720-736 |
| | INTEGRAL | Likelihood = | −4.83 | Transmembrane | 742-758 |
| | INTEGRAL | Likelihood = | −10.83 | Transmembrane | 775-791 |
| | PERIPHERAL | Likelihood = | 5.25 (at 600) | | |

| ALOM score: −10.83 (number of TMSs: 7) | | | |
|---|---|---|---|
| MTOP: Prediction of membrane topology (Hartmann et al.) | | | |
| Center position for calculation: 388 | | | |
| Charge difference: 0.5 C(1.5)-N(1.0) | | | |
| C > N: C-terminal side will be inside | | | |
| >>>Caution: Inconsistent mtop result with signal peptide | | | |
| >>>membrane topology: type 3b | | | |
| MITDISC: discrimination of mitochondrial targeting seq | | | |
| R content: | 2 | Hyd Moment (75): | 6.30 |
| Hyd Moment (95): | 5.24 | G content: | 2 |
| D/E content: | 2 | S/T content: | 10 |

Score: −3.23
Gavel: prediction of cleavage sites for mitochondrial preseq
R-2 motif at 32 SRK|PP
NUCDISC: discrimination of nuclear localization signals
pat4: PRRK (4) at 204
pat4: RRKR (5) at 205
pat4: RKKR (5) at 335
pat4: KKRH (3) at 336
pat7: PRRKRMS (5) at 204
pat7: PGPRRGK (3) at 316
pat7: PRRGKRI (5) at 318
bipartite: KRIASKVKHFAFDRKKR at 322
content of basic residues: 11.6%
NLS Score: 2.37
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 70.6
COIL: Lupas's algorithm to detect coiled-coil regions
total: 0 residues
Final Results (k = 9/23):
55.6%: endoplasmic reticulum
11.1%: vacuolar
11.1%: mitochondrial
11.1%: vesicles of secretory system
11.1%: Golgi
>> prediction for CG52414-02 is end (k = 9)

A search of the NOV16a protein against the Geneseq database, a proprietary database that contains sequences published in patents and patent publication, yielded several homologous proteins shown in Table 16D.

TABLE 16D
Geneseq Results for NOV16a

| | | NOV16a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAB61148 | Human NOV17 protein - Homo | 120 . . . 827 | 705/708 (99%) | 0.0 |
| | sapiens, 708 aa. [WO200075321-A2, | 1 . . . 708 | 705/708 (99%) | |
| | 14 DEC. 2000] | | | |
| AAB61147 | Human NOV16 protein - Homo | 120 . . . 604 | 484/485 (99%) | 0.0 |
| | sapiens, 578 aa. [WO200075321-A2, | 1 . . . 485 | 484/485 (99%) | |
| | 14 DEC. 2000] | | | |
| ABG64458 | Human albumin fusion protein #1133 - | 498 . . . 827 | 328/330 (99%) | 0.0 |
| | Homo sapiens, 349 aa. | 20 . . . 349 | 330/330 (99%) | |
| | [WO200177137-A1, 18 OCT. 2001] | | | |
| AAE03323 | Human gene 7 encoded secreted | 498 . . . 827 | 328/330 (99%) | 0.0 |
| | protein HCRNC80, SEQ ID NO: 97 - | 20 . . . 349 | 330/330 (99%) | |
| | Homo sapiens, 349 aa. | | | |
| | [WO200134800-A1, 17 MAY 2001] | | | |
| ABB90342 | Human polypeptide SEQ ID NO 2718 - | 505 . . . 827 | 322/323 (99%) | 0.0 |
| | Homo sapiens, 323 aa. | 1 . . . 323 | 323/323 (99%) | |
| | [WO200190304-A2, 29 NOV. 2001] | | | |

In a BLAST search of public sequence databases, the NOV16a protein was found to have homology to the proteins shown in the BLASTP data in Table 16E.

TABLE 16E
Public BLASTP Results for NOV16a

| | | NOV16a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q8BJ70 | Epidermal growth factor receptor- | 1 . . . 827 | 759/830 (91%) | 0.0 |
| | related protein homolog - Mus | 1 . . . 827 | 790/830 (94%) | |
| | musculus (Mouse), 827 aa. | | | |
| CAC22528 | Sequence 33 from Patent | 120 . . . 827 | 705/708 (99%) | 0.0 |
| | WO0075321 - Homo sapiens | 1 . . . 708 | 705/708 (99%) | |
| | (Human), 708 aa. | | | |
| Q9H6E9 | Hypothetical protein FLJ22341 - | 209 . . . 827 | 619/619 (100%) | 0.0 |
| | Homo sapiens (Human), 619 aa. | 1 . . . 619 | 619/619 (100%) | |
| BAB84860 | FLJ00080 protein - Homo sapiens | 80 . . . 689 | 603/613 (98%) | 0.0 |
| | (Human), 716 aa (fragment). | 52 . . . 664 | 605/613 (98%) | |
| Q8K2I7 | Similar to hypothetical protein | 222 . . . 827 | 564/608 (92%) | 0.0 |
| | FLJ22341 - Mus musculus | 1 . . . 607 | 585/608 (95%) | |
| | (Mouse), 607 aa (fragment). | | | |

PFam analysis predicts that the NOV16a protein contains the domains shown in the Table 16F.

TABLE 16F
Domain Analysis of NOV16a

| | | Identities/ | |
|---|---|---|---|
| | | Similarities | |
| Pfam | NOV16a | for the | Expect |
| Domain | Match Region | Matched Region | Value |
| Rhomboid | 619 . . . 763 | 56/171 (33%) | 7.9e−44 |
| | | 127/171 (74%) | |

### Example 17

The NOV17 clone was analyzed, and the nucleotide and encoded polypeptide sequences are shown in Table 17A.

TABLE 17A
NOV17 Sequence Analysis

NOV17a,
CG52643-02 SEQ ID NO: 239 5573 bp
DNA Sequence ORF Start: ATG at 408 ORF Stop: TGA at 2934
GGACAGGGCTGCATTGCTGTTGCTCACTGACCTTCTTTTATGCTGCCCTTTGGTTCAGAATGGCACAT
CATTCCTCGTTTTTGGCCCTCCAGCTGAACACCTGTTCTCTGTGCCACTGACTCCTCTTTCCATAGGG
ACATCATACAACAGTCGCCTTTATCTGAGGTTGTGCAAAGAGGGATGGAGGAGAAAACAATGGAGAAT
CCCTGGCAGATTTCCCCAGGACGAGAGAAGGATATCCAATTGCTCATCAGGGAAGGTGCTAGGTCTCC
CAGCCAGACGCCCTCAGAGGCCGGTGTCAAGTCTCCCTCACCTCTGTGATGTGAAGTCAGCTCGTTCA
TGACCTCCGCAGGCAGAGGGTCAGAGGGGCAGATGGACCACTCCTGGCCTGATCAAGACTCATCAAAA
TGAAACCAGGAGGCTTTTGGCTGCATCTCACACTGCTCGGAGCCTCCCTGCCGGCTCCGCTGGGATGG
ATGGACCCAGGAACCAGCAGAGCCCCGGATCTGGGTGTGGGGGAGTCACAGGCACAGGAGCCCAGAAG
CTTTGAAGTCACAAGAAGAGAAGGGCTTTCCAGCCACAACGAGCTGCTGGCCTCCTGCGGGAAGAAGT
TCTGCACCCGAGGGAGCCGGTGCGTGCTCAGCAGGAAGACAGGGGAGCCCGAATGCCAGTGCCTGGAG
GCATGCAGGCCCAGCTACGTGCCTGTGTGCGGCTCTGATGGGAGGTTTTATGAAAACCACTGTAAGCT
CCACCGTGCTGCTTGCCTCCTGGGAAAGAGGATCACCGTCATCCACAGCAAGGACTGTTTCCTCAAAG
GTGACACGTGCACCATGGCCGGCTACGCCCGCTTGAAGAATGTCCTTCTGGCACTCCAGACCCGTCTG
CAGCCACTCCAAGAAGGAGACAGCAGACAAGACCCTGCCTCCCAGAAGCGCCTCCTGGTGGAATCTCT
GTTCAGGGACTTAGATGCAGATGGCAATGGCCACCTCAGCAGCTCCGAACTGGCTCAGCATGTCCTGA
AGAAGCAGGACCTGGATGAAGACTTACTTGGTTGCTCACCAGGTGACCTCCTCCGATTTGACGATTAC
AACAGTGACAGCTCCCTGACCCTCCGCGAGTTCTACATGGCCTTCCAAGTGGTTCAGCTCAGCCTCGC
CCCCGAGGACAGGGTCAGTGTGACCACACTCACCCTGGGCCTGAGCACAGTGCTGACCTGCGCCGTCC
ATGGAGACCTGAGGCCACCAAATCATCTGGAGCGCAACGGGCTCACCCTGAACTTCCTGGACTTGGAA
GACATCAATGACTTTGGAGAGGATGATTCCCTGTACATCACCAAGGTGACCACCATCCACATGGGCAA
TTACACCTGCCATGCTTCCGGCCACGAGCAGCTGTTCCAGACCCACGTCCTGCAGGTGAATGTGCCGC
CAGTCATCCGTGTCTATCCAGAGAGCCAGGCACAGGAGCCTGGAGTGGCAGCCAGCCTAAGATCCCAT
GCTGAGGCCATTCCCATGCCCAGAATCACTTGGCTGAAAAACGGCGTGGATGTCTCAACTCAGATGTC
CAAACAGCTCTCCCTTTTAGCCAATGGGAGCGAACTCCACATCAGCAGTGTTCGGTATGAAGACACAC
GGGCATACACCTGCATTGCCAAAAATGAAGTGGGTGTGGATGAAGATATCTCCTCGCTCTTCATTGAA
GACTCAGCTAGAAAGACCCTTGCAAACATCCTGTGGCGAGAGGAAGGCCTCAGCGTGGGAAACATGTT
CTATGTCTTCTCCCACGACGCTATCATCGTCATCCATCCTGTGGACTGTGAGATCCAGAGGCACCTCA
AACCCACGGAAAACATTTTCATGAGCTATGAAGAAATCTGTCCTCAAAGAGAAAAAAATGCAACCCAG
CCCTGCCAGTGGGTATCTGCAGTCAATGTCCGGAACCGGTACATCTATGTGGCCCAGCCAGCACTGAG
CAGAGTCCTTGTCGTCGACATCCAAGCCCAGAAAGTCCTACAGTCCATAGGTGTGGACCCTCTGCCGG
CTAAGCTGTCCTATGACAAGTCACATGACCAAGTGTGGGTCCTCAGCTGGGCGGACGTGCACAAGTCC
CGACCAAGTCTCCAGGTGATCACAGAAGCCAGCACCGGCCAGAGCCAGCACCTCATCCGCACACCCTT
TGCAGGAGTGGATGATTTCTTCATTCCCCCAACAAACCTCATCATCAACCACATCAGGTTTGGCTTCA
TCTTCAACAAGTCTGATCCTGCAGTCCACAAGGTGGACCTGGAAACAATGATGCCCCTCAAGACCATC
GGCCTGCACCACCATGGCTGCGTGCCCCAGGCCATGGCACACACCCACCTGGGCGGCTACTTCTTCAT
CCAGTGCCGACAGGACAGCCCCGCCTCTGCTGCCCGACAGCTGCTCGTTGACAGTGTCACAGACTCTG
TGCTTGGCCCCAATGGTGATGTAACAGGCACCCCACACACATCCCCCGACGGGCGCTTCATAGTCAGT
GCTGCAGCTGACAGCCCCTGGCTGCACGTGCAGGAGATCACAGTGCGGGGCGAGATCCAGACCCTGTA
TGACCTGCAAATAAACTCGGGCATCTCAGACTTGGCCTTCCACCGCTCCTTCACTGAAAGCAATCAAT
ACAACATCTACGCGGCTCTGCACACGGAGCCGGACCTGCTGTTCCTGGAGCTGTCCACGGGGAAGGTG
GGCATGCTGAAGAACTTAAAGGAGCCACCCGCAGGGCCAGCTCAGCCCTGGGGGGGTACCCACAGAAT
CATGAGGGACAGTGGGCTGTTTGGACAGTACCTCCTCACACCAGCCCGAGAGTCACTGTTCCTCATCA
ATGGGAGACAAAACACGCTGCGGTGTGAGGTGTCAGGTATAAAGGGGGGGACCACAGTGGTGTGGGTG
GGTGAGGTATGAAGGGCCCAGAGCAGAGCCCTGGGCCAAGGAACACCCCCTAGTCCTGACACTGCAGC
CTCAAGCAGGTACGCTGTACATTTTTACAGACAAAAGCAAAAACCTGTACTCGCTTTGTGGTTCAACA
CTGGTCTCCTTGCAAGTTTCCTAGTATAAGGTATGCGCTGCTACCAAGATTGGGGTTTTTTCGTTAGG
AAGTATGATTTATGCCTTGAGCTACGATGAGAACATATGCTGCTGTGTAAAGGGATCATTTCTGTGCC
AAGCTGCACACCGAGTGACCTGGGGACATCATGGAACCAAGGGATCCTGCTCTCCAAGCAGACACCTC
TGTCAGTTGCCTTCACATAGTCATTGTCCCTTACTGCCAGACCCAGCCAGACTTTGCCCTGACGGAGT
CGCCCGGAAGCAGAGGCCGACCAGGAGCAGGGGCCTCCCTCCCGAACTGAAAGCCCATCCGTCCTCGC
GTGGGACCGCATCTTCTCCCTCGCACCTGCTTCTTCCTTTTCTTTCCATTTGACTTGCTGTAAGCCTG
AGGGAGAGCCAACAAGACTTACTGCATCTTGGGGGATGGGCAAATCACTCACTTTATTTTGGAAATTT
TTGATTAAAAAAAAATTTTATAATCTCAAATGCTAGTAAGCAGAAAGATGCTCTCCCAGGTCCAACTA
TATCCTTCCCTGCCTTAGGCCGAGTCTCGGGGGTGGTCACAACCCCACATCCCACAGCCAGAAAGAAC
AATGGTCATCTGAGAATACTGGCCCTGTCGACTATTGCCACCCTGCTTCTCCAAGAGCAGACCAGGCC
ACCTCATCCGTAAGGACTCGGTTCTGTGTTGGGACCCCAAAAAACCAGAACAAGTTCTGTGTGCCTCC
TTTCACCACAGAAGGGAGACATCTCATTAGTCAGGTCTGGTACCCCAGATTCAGGGCAGACTGGGCTT
CCCTGGCAAGGTATGGGTGGCCTCCAGGCTCAATGCAGAAACCCCAAGGACACGAGTGGGGCCAGGTG
AGTTCCTGAAGCTATACCTTTTCAAAACAGATTTTGTTTTCCTACCTGTGGCCCATCCACTCCTCTCT
GGTACCCCATCCCCGCATCAGCACTGCAGAGAGAACACATTTCGGCGAGGGTTTTCTTACCCACATTC
CCCAATCAATACACACACACTCCAGAACCCAGAACAGAAGGCCACAGGCTGGCACTACTGCATTCTCC
TTATGTGTCTCAGGCTGTGGTGACTCTCACATGGGCATCGAAGAAGTACAACCCACATAGCCCTCTGG
AGACCGCCTAGATCAGAGACTCAGCAAAAACAGGCTCGCCTTCCCTCTCCCACATATGAGTGGAACTT
ACATGTGTCCTGCTTTCAATGATCATTTTGCAAGCCACACGGGTTGGGAGAGGTGGTCTCACCACAGA
CGTCTTTCCTAATTTGUCCACCTTCACCTACTGACATGACCAGCATTTTCCTTTGCCATTAAGGAATG
AACTCTTTCAAGGAGAGGAAACCCTAGACTCTGTGTCACTCTCAACACACACAGCTCCTTTCACTCCT
GCCTGACTGCCAAGCCACCTGCATCCCCCGCCCCAGATCTCATGAGATCAATCACTTGTATGTCTCAC
GCAACTTCGTCCACCAAACGCCTGTCCCCTGTAACTCCTAGGGGTGCGCCTAGACAGGTACGTCTGTT
TTTTATTTTAAAAGATATGCTATGTAGATATAAGTTGAGGAAGCTCACCTCAAAAGCCTAGAATGCAG
TTTCACAGTACCTGCGATGCATGGATGACCCATCTCACCCCTTTTTTTTTCCTGCCTCAATATCTTGA
TATGTTATGTTTACTCCCAATCTCCCATTTTTACCACTAAAATTCTCCAACTTTCATAAACTTTTTTT
TGGAAAAATTTCCATTGTATCAGCCCCTGACAGAAAAAGGATCTCTGAGCCTAAAGGAGCAAAAGTCC
CACCAACTACCAGACCAGAACACGAGCCCCTCTGGGCAGCAGGATTCCTAAGTCAAAGACCAGTTTGA
CCCAAACTGGCCTTTTAAAATAATCAGGAGTGACAGAGTCAACTTCTGCAGCACCTGCTTCTCCCCCA
CTGTCCCTTCCATCTTGGAATGTGTCTAAAAAAGCATAGCTGCCCTTTGCTGTCCTCAGAGTGCATTT
CCTGGAGACGGCAGGCTTAGGTCTCACTGACAGCATGCCAGACACAACTGAATCGAACCAGGCCTGAA
GCCTAGGTCAGGGTTTCAGGAGTCCAGCCCCAGGAGGCAAAGTCACCAATGCAGGGAGCTAAATGCCT
TTTGGCAGGAAAACCAATAGAGTTGGTTGGGTGGGGAGTCAGGGGTGGGAGGAGAAGGAGGAAGAGGA
GGAACGCCAGACTGGCCTGCCCTTTCTCCCATACTTCACCCCAGCAGAGGTTCATGGGACACAGTTGG
AAAGCCACTGGGAGGAAATGCCTCACTACAGGGGGGCCTCCTGTAGCAAGCCCAGCCGGTAATCCTCC
TAATGAACCCACAAGGTCAATTCACAACTGATATCTTAGCTATTAAAGAAGTACTGACTTTACCAAAA
GAATCATCAAGAAAGCTATTTATATAAACCCCCTCAGTCATTTTGAAATAAAATTAATTTTACAA
NOV17a,
CG52643-02
Protein Sequence SEQ ID NO: 240 842 aa MW at 93094.8 kD
MKPGGFWLHLTLLGASLPAALGWMDPGTSRGPDVGVGESQAEEPRSFEVTRREGLSSHNELLASCGKK
FCSRGSRCVLSRKTGEPECQCLEACRPSYVPVCGSDGRFYENHCKLHRAACLLCKRITVIHSKDCFLK
GDTCTMACYARLKNVLLALQTRLQPLQECDSRQDPASQKRLLVESLFRDLDADGNGHLSSSELAQHVL
KKQDLDEDLLGCSPGDLLRFDDYNSDSSLTLREFYMAFQVVQLSLAPEDRVSVTTVTVGLSTVLTCAV
HGDLRPPIIWKRNGLTLNFLDLEDINDFGEDDSLYITKVTTIHMGNYTCHASGHEQLFQTHVLQVNVP
PVIRVYPESQAQEPGVAASLRCNAEGIPMPRITWLKNGVDVSTQMSKQLSLLANGSELHISSVRYEDT
GAYTCIAKNEVGVDEDISSLFIEDSARKTLANILWREEGLSVGNMFYVFSDDGIIVIHPVDCEIQRHL
KPTEKIFMSYEEICPQREKNATQPCQWVSAVNVRNRYIYVAQPALSRVLVVDIQAQKVLQSIGVDPLP
AKLSYDKSHDQVWVLSWGDVHKSRPSLQVITEASTGQSQHLIRTPFAGVDDFFIPPTNLIINHIRFGF
IFNKSDPAVHKVDLETMMPLKTIGLHHHGCVPQAMAHTHLGGYFFIQCRQDSPASAARQLLVDSVTDS
VLGPNGDVTGTPHTSPDGRFIVSAAADSPWLNVQEITVRGEIQTLYDLQINSCISDLAFQRSFTESNQ
YNIYAALHTEPDLLFLELSTGKVGMLKNLKEPPAGPAQPWGCTHRIMRDSGLFGQYLLTPARESLFLI
NGRQNTLRCEVSCIKGGTTVVWVGEV
NOV17b,
259341359 SEQ ID NO: 241 2538 bp
DNA Sequence ORF Start: at 1 ORF Stop: end of sequence
CTCGAGATGAAACCAGGAGGCTTTTCGCTGCATCTCACACTGCTCGGAGCCTCCCTGCCGGCTGCGCT
GGGATGGATGGACCCAGGAACCAGCAGAGGCCCGGATGTGGGTGTGCGGGAGTCACAGGCAGAGGAGC
CCAGAAGCTTTGAAGTCACAAGAAGAGAAGGGCTTTCCAGCCACAACGAGCTGCTGGCCTCCTGCGGG
AAGAAGTTCTGCAGCCGAGGGAGCCGGTGCGTGCTCAGCAGGAAGACAGGGGAGCCCGAATGCCAGTG
CCTGGAGGCATGCAGGCCCAGCTACGTCCCTCTGTGCGGCTCTGATGGGAGGTTTTATGAAAACCACT
GTAAGCTCCACCGTGCTGCTTCCCTCCTGGGAAAGAGGATCACCGTCATCCACAGCAAGGACTGTTTC
CTCAAAGGTGACACGTGCACCATGGCCGGCTACGCCCGCTTGAAGAATGTCCTTCTGGCACTCCAGAC
CCGTCTGCAGCCACTCCAAGAAGGAGACAGCAGACAAGACCCTGCCTCCCAGAAGCGCCTCCTGGTCG
AATCTCTGTTCAGGGACTTAGATGCAGATGGCAATGGCCACCTCAGCAGCTCCGAACTGCCTCAGCAT
GTGCTGAAGAAGCAGGACCTGGATGAAGACTTACTTGGTTGCTCACCAGGTGACCTCCTCCGATTTGA
CGATTACAACAGTGACAGCTCCCTGACCCTCCGCGAGTTCTACATGGCCTTCCAAGTGGTTCAGCTCA
GCCTCGCCCCCGAGGACAGGGTCAGTGTGACCACAGTGACCGTGGGGCTGAGCACAGTGCTGACCTGC
GCCGTCCATGCAGACCTGAGGCCACCAATCATCTGGAAGCGCAACGGGCTCACCCTGAACTTCCTGGA
CTTGGAAGACATCAATGACTTTGGAGAGGATGATTCCCTGTACATCACCAAGGTGACCACCATCCACA
TGGCCAATTACACCTGCCATGCTTCCGGCCACGAGCAGCTGTTCCAGACCCACGTCCTGCAGGTGAAT
GTGCCGCCAGTCATCCGTGTCTATCCAGAGAGCCAGGCACAGGAGCCTGGAGTGGCAGCCAGCCTAAG
ATGCCATGCTGAGGGCATTCCCATGCCCAGAATCACTTGGCTGAAAAACGGCGTGGATGTCTCAACTC
AGATGTCCAAACAGCTCTCCCTTTTAGCCAATGGGAGCGAACTCCACATCAGCAGTGTTCGGTATGAA
GACACAGGGGCATACACCTGCATTGCCAAAAATGAAGTGGGTGTGGATGAAGATATCTCCTCGCTCTT
CATTGAAGACTCAGCTAGAAAGACCCTTGCAAACATCCTGTGGCGAGAGGAAGGCCTCAGCGTCGGAA
ACATGTTCTATGTCTTCTCCGACGACGGTATCATCGTCATCCATCCTGTGGACTGTGACATCCACAGG
CACCTCAAACCCACGGAAAAGATTTTCATGAGCTATGAAGAAATCTGTCCTCAAAGAGAAAAAAATGC
AACCCAGCCCTGCCAGTGGGTATCTGCAGTCAATGTCCGGAACCGGTACATCTATGTGGCCCAGCCAG
CACTGAGCAGAGTCCTTGTGGTCGACATCCAAGCCCACAAAGTCCTACAGTCCATAGGTGTGGACCCT
CTGCCGGCTAAGCTGTCCTATGACAAGTCACATGACCAAGTGTGCGTCCTGAGCTGGGGGGACGTGCA
CAAGTCCCGACCAAGTCTCCAGGTCATCACAGAAGCCAGCACCGGCCAGAGCCAGCACCTCATCCGCA
CACCCTTTGCAGGAGTGGATGATTTCTTCATTCCCCCAACAAACCTCATCATCAACCACATCAGGTTT
GGCTTCATCTTCAACAAGTCTGATCCTGCAGTCCACAAGGTGGACCTGGAAACAATGATGCCCCTCAA
GACCATCGGCCTGCACCACCATGGCTGCGTGCCCCAGGCCATGGCACACACCCACCTGGGCGGCTACT
TCTTCATCCAGTGCCGACAGGACAGCCCCGCCTCTGCTGCCCGACAGCTGCTCGTTGACAGTGTCACA
GACTCTGTGCTTGGCCCCAATGGTGATGTAACAGGCACCCCACACACATCCCCCCACGGGCGCTTCAT
AGTCAGTGCTGCAGCTGACAGCCCCTGCCTGCACGTGCAGGAGATCACAGTGCGGGGCGAGATCCAGA
CCCTGTATGACCTGCAAATAAACTCGGGCATCTCAGACTTGGCCTTCCAGCGCTCCTTCACTGAAAGC
AATCAATACAACATCTACGCGGCTCTGCACACCGAGCCGGACCTGCTGTTCCTGGAGCTGTCCACGGG
GAAGGTGGGCATGCTGAAGAACTTAAAGGAGCCACCCGCAGGGCCAGCTCAGCCCTGCGGGGGTACCC
ACAGAATCATGAGGGACAGTGGGCTGTTTGGACAGTACCTCCTCACACCAGCCCGAGAGTCACTGTTC
CTCATCAATGGGAGACAAAACACGCTGCGGTGTGAGGTGTCAGGTATAAAGGGGCCGACCACAGTGGT
GTCGGTGGGTGAGGTACTCGAG
NOV17b,
259341359
Protein Sequence SEQ ID NO: 242 846 aa MW at 93579.4 kD
LEMKPGGFWLHLTLLGASLPAALGWMDPGTSRGPDVGVGESQAEEPRSFEVTRREGLSSHNELLASCG
KKFCSRGSRCVLSRKTGEPECQCLEACRPSYVPVCGSDGRFYENHCKLHRAACLLGKRITVIHSKDCF
LKGDTCTMAGYARLKNVLLALQTRLQPLQECDSRQDPASQKRLLVESLFRDLDADGNGHLSSSELAQH
VLKKQDLDEDLLGCSPGDLLRFDDYNSDSSLTLREFYMAFQVVQLSLAPEDRVSVTTVTVGLSTVLTC
AVHGDLRPPIIWKRNGLTLNFLDLEDINDFGEDDSLYITKVTTIHMGNYTCHASGHEQLFQTHVLQVN
VPPVIRVYPESQAQEPGVAASLRCHAEGIPMPRITWLKNGVDVSTQMSKQLSLLANGSELHISSVRYE
DTGAYTCIAKNEVGVDEDISSLFIEDSARKTLANILWREEGLSVGNMFYVFSDDGIIVIHPVDCEIQR
HLKPTEKIFMSYEEICPQREKNATQPCQWVSAVNVRNRYIYVAQPALSRVLVVDIQAQKVLQSIGVDP
LPAKLSYDKSHDQVWVLSWGDVHKSRPSLQVITEASTGQSQHLIRTPFAGVDDFFIPPTNLIINHIRF
GFIFNKSDPAVHKVDLETMMPLKTIGLHHHGCVPQAMAHTHLGGYFFIQCRQDSPASAARQLLVDSVT
DSVLGPNGDVTGTPHTSPDGRFIVSAAADSPWLHVQEITVRGEIQTLYDLQINSGISDLAFQRSFTES
NQYNIYAALHTEPDLLFLELSTGKVGMLKNLKEPPAGPAQPWGGTHRIMRDSGLFGQYLLTPARESLF
LINGRQNTLRCEVSGIKGGTTVVWVGEVLE
NOV17c,
268824728 SEQ ID NO: 243 2511 bp
DNA Sequence ORF Start: at 1 ORF Stop: end of sequence
CTCGACATGAAACCAGGAGGCTTTTGGCTGCATCTCACACTGCTCGGAGCCTCCCTGCCGGCTCCGCT
GGGATGGATGGACCCAGGAACCAGCAGAGGCCCGGATGTGGGTGTGGGGGAGTCACAGGCAGAGGAGC
CCAGAAGCTTTGAAGTCACAAGAAGAGAAGGGCTTTCCAGCCACAACGAGCTGCTGGCCTCCTGCGGG
AAGAAGTTCTGCAGCCGAGGGAGCCGGTGCGTGCTCAGCAGGAAGACAGGCGAGCCCGAATGCCAGTG
CCTGGAGGCATGCAGGCCCAGCTACGTGCCTGTGTGCGGCTCTGATGGGACGTTTTATGAAAACCACT
GTAAGCTCCACCGTGCTGCTTGCCTCCTGGGAAAGAGGATCACCGTCATCCACAGCAAGGACTGTTTC
CTCAAAGGTGACACGTGCACCATTGCCGGCTACGCCCGCTTGAAGAATGTCCTTCTGGCACTCCAGAC
CCGTCTGCAGCCACTCCAAGAAGGAGACAGCAGACAAGACCCTGCCTCCCAGAAGCGCCTCCTGGTGG
AATCTCTGTTCAGGGACTTAGATGCAGATGGCAATGGCCACCTCAGCAGCTCCGAACTGGCTCAGCAT
GTGCTGAAGAAGCACGACCTGGATGAAGACTTACTTGGTTGCTCACCAGGTGACCTCCTCCGATTTGA
CGATTACAACAGTGACAGCTCCCTGACCCTCCGCGAGTTCTACATGGCCTTCCAAGTGGTTCAGCTCA
GCCTCGCCCCCGAGGACAGGGTCAGTGTGACCACAGTGACCGTCGCGCTGAGCACAGTGCTGACCTGC
GCCGTCCATGGAGACCTGAGGCCACCAATCATCTGGAAGCGCAACGCGCTCACCCTGAACTTCCTGGA
CTTGGAAGACATCAATGACTTTGGAGAGGATGATTCCCTGTACATCACCAAGGTGACCACCATCCACA
TGGGCAATTACACCTGCCATGCTTCCGGCCACGAGCAGCTGTTCCAGACCCACGTCCTGCAGGTGAAT
GTGCCGCCAGTCATCCGTGTCTATCCAGAGAGCCAGGCACAGGAGCCTGGAGTGGCAGCCAGCCTAAG
ATGCCATGCTGAGGCCATTCCCATGCCCAGAATCACTTGGCTGAAAAACGGCGTGGATGTCTCAACTC
AGATGTCCAAACAGCTCTCCCTTTTACCCAATGGGAGCGAACTCCACATCAGCAGTGTTCGGTATGAA
GACACAGGGGCATACACCTGCATTGCCAAAAATGAAGTGGGTGTGGATGAAGATATCTCCTCGCTCTT
CATTGAAGACTCAGCTAGAAAGACCCGCCTCAGCGTGGGAAACATGTTCTATGTCTTCTCCGACGACG
GTATCATCGTCATCCATCCTGTGGACTGTGAGATCCAGAGGCACCTCAAACCCACGGAAAAGATTTTC
ATGAGCTATGAAGAAATCTGTCCTCAAAGAGAAAAAAATGCAACCCAGCCCTGCCAGTGGGTATCTGC
AGTCAATGTCCGGAACCGGTACATCTATGTGGCCCAGCCACCACTGAGCAGAGTCCTTGTGGTCGACA
TCCAAGCCCAGAAAGTCCTACAGTCCATAGGTGTGGACCCTCTCCCGGCTAACCTGTCCTATGACAAG
TCACATGACCAAGTGTGGGTCCTGAGCTGGGGGGACGTGCACAAGTCCCGACCAAGTCTCCAGGTGAT
CACAGAAGCCAGCACCGGCCAGAGCCAGCACCTCATCCGCACACCCTTTGCAGGAGTCGATGATTTCT
TCATTCCCCCAACAAACCTCATCATCAACCACATCAGGTTTGGCTTCATCTTCAACAAGTCTGATCCT
GCAGTCCACAAGGTGGACCTGGAAACAATGATGCCCCTCAAGACCATCGGCCTGCACCACCATGGCTG
CCTCCCCCAGGCCATGGCACACACCCACCTGGGCGGCTACTTCTTCATCCAGTCCCGACAGGACAGCC
CCUCCTCTGCTGCCCCACAGCTGCTCGTTGACAGTGTCACAGACTCTGTCCTTGGCCCCAATGGTGAT
GTAACAGGCACCCCACACACATCCCCCGACGGGCGCTTCATAGTCAGTGCTGCAGCTGACAGCCCCTG
GCTGCACGTGCAGGAGATCACAGTGCGGGGCGAGATCCAGACCCTGTATGACCTGCAAATAAACTCGG
GCATCTCAGACTTGGCCTTCCAGCGCTCCTTCACTGAAAGCAATCAATACAACATCTACGCGGCTCTG
CACATGGAGCCCGACCTGCTGTTCCTGGAGCTGTCCACGGGGAAGGTGCGCATGCTGAAGAACTTAAA
GGACCCACCCGCAGGGCCAGCTCAGCCCTGGGGGGGTACCCACAGAATCATGAGGGACAGTGGGCTGT
TTGGACAGTACCTCCTCACACCAGCCCGAGAGTCACTGTTCCTCATCAATGGGAGACAAAACACGCTG
CGGTGTGAGGTGTCAGGTATAAAGGGGGGGACCACAGTGGTGTGGGTGGGTGAGGTACTCGAG
NOV17c,
268824728
Protein Sequence SEQ ID NO: 244 837 aa MW at 92565.3 kD
LEMKPGGFWLHLTLLGASLPAALGWMDPGTSRGPDVGVGESQAEEPRSFEVTRREGLSSHNELLASCG
KKFCSRGSRCVLSRKTGEPECQCLEACRPSYVPVCGSDGRFYENHCKLHRAACLLGKRITVIHSKDCF
LKGDTCTIAGYARLKNVLLALQTRLQPLQEGDSRQDPASQKRLLVESLFRDLDADGNGHLSSSELAQH
VLKKQDLDEDLLGCSPGDLLRFDDYNSDSSLTLREFYMAFQVVQLSLAPEDRVSVTTVTVGLSTVLTC
AVHGDLRPPIIWKRNGLTLNFLDLEDINDFGEDDSLYITKVTTIHMGNYTCHASGHEQLFQTHVLQVN
VPPVIRVYPESQAQEPGVAASLRCHAEGIPMPRITWLKNGVDVSTQMSKQLSLLANGSELHISSVRYE
DTGAYTCILAKNEVGVDEDISSLFIEDSARKTRLSVGNMFYVFSDDGHVIHPVDCEIQRHLKPTEKIF
MSYEEICPQREKNATQPCQWVSAVNVRNRYIYVAQPALSRVLVVDIQAQKVLQSIGVDPLPAKLSYDK
SHDQVWVLSWGDVHKSRPSLQVITEASTGQSQHLIRTPFAGVDDFFIPPTNLIINHIRFGFIFNKSDP
AVHKVDLETMMPLKTIGLHHHGCVPQAMAHTHLGGYFFIQCRQDSPASAARQLLVDSVTDSVLGPNGD
VTGTPHTSPDGRFIVSAAADSPWLHVQEITVRGEIQTLYDLQINSGISDLAFQRSFTESNQYNIYAAL
HMEPDLLFLELSTGKVGMLKNLKEPPAGPAQPWGGTHRIMRDSGLFGQYLLTPARESLFLINGRQNTL
RCEVSGIKGGTTVVWVGEVLE
NOV17d,
268825987 SEQ ID NO: 245 2439 bp
DNA Sequence ORF Start at 1 ORF Stop: end of sequence
CTCGAGATGAAACCAGCAGGCTTTTGGCTGCATCTCACACTGCTCGGAGCCTCCCTGCCGGCTGCGCT
GGGATGGATGGACCCAGGAACCAGCAGAGGCCCGGATGTGGGTGTGGGGGAGTCACAGGCAGAGGACC
CCAGAAGCTTTGAAGTCACAAGAAGAGAAGGGCTTTCCAGCCACAACGAGCTGCTGGCCTCCTGCGGG
AAGAAGTTCTGCAGCCGAGGGAGCCGGTGCGTGCTCAGCAGGAAGACAGGGGAGCCCGAATGCCTGGG
AAAGAGGATCACCGTCATCCACAGCAAGGACTGTTTCCTCAAAGGTGACACGTGCACCATGGCCGGCT
ACGCCCCCTTGAAGAATGTCCTTCTGGCACTCCAGACCCGTCTGCAGCCACTCCAAGAAGGAGACAGC
AGACAAGACCCTGCCTCCCAGAAGCGCCTCCTGGTGGAATCTCTGTTCACGCACTTAGATGCAGATGG
CAATGGCCACCTCAGCAGCTCCGAACTGGCTCAGCATGTGCTGAAGAAGCAGGACCTGGATGAAGACT
TACTTGGTTGCTCACCAGGTGACCTCCTCCGATTTGACGATTACAACAGTGACAGCTCCCTGACCCTC
CGCGAGTTCTACATGGCCTTCCAAGTGGTTCAGCTCAGCCTCGCCCCCGAGGACAGGGTCAGTGTGAC
CACAGTGACCGTGGGGCTGAGCACAGTGCTGACCTGCGCCGTCCATGGAGACCTGAGGCCACCAATCA
TCTGGAGCGCAACGGGCTCACCCTGAACTTCCTGGACTTGGAAGACATCAATGACTTTGCAAGAGGAT
GATTCCCTGTACATCACCAAGGTGACCACCATCCACATGGGCAATTACACCTGCCATGCTTCCGGCCA
CGAGCAGCTGTTCCAGACCCACGTCCTGCAGGTGAATGTGCCGCCAGTCATCCGTGTCTATCCAGAGA
GCCAGGCACAGGAGCCTGGAGTGGCAGCCAGCCTAAGATGCCATGCTGAGGGCATTCCCATGCCCAGA
ATCACTTGGCTGAAAAACGGCGTGGATGTCTCAACTCAGATGTCCAAACAGCTCTCCCTTTTAGCCAA
TGGGAGCGAACTCCACATCAGCAGTGTTCGGTATGAAGACACAGGGGCATACACCTGCATTGCCAAAA
ATGAAGTGGGTGTGGATGAAGATATCTCCTCGCTCTTCATTGAAGACTCAGCTAGAAAGACCCTTGCA
AACATCCTGTGGCGAGAGGAAGGCCTCAGCGTGGGAAACATGTTCTATGTCTTCTCCGACGACGGTAT
CATCGTCATCCATCCTGTCGACTGTGAGATCCAGAGGCACCTCAAACCCACGGAAAAGATTTTCATGA
GCTATGAAGAAATCTGTCCTCAAAGAGAAAAAAATGCAACCCAGCCCTGCCAGTGGGTATCTGCAGTC
AATGTCCGGAACCGGTACATCTATGTGGCCCACCCAGCACTGAGCAGAGTCCTTGTGGTCGACATCCA
AGCCCAGAAAGTCCTACAGTCCATAGGTGTGGACCCTCTGCCGGCTAAGCTGTCCTATGACAAGTCAC
ATGACCAAGTGTGGGTCCTGAGCTGGGGGGACGTGCACAAGTCCCGACCAAGTCTCCAGGTGATCACA
GAAGCCAGCACCGGCCAGAGCCAGCACCTCATCCGCACACCCTTTGCAGGAGTGGATGATTTCTTCAT
TCCCCCAACAAACCTCATCATCAACCACATCAGGTTTGGCTTCATCTTCAACAAGTCTGATCCTGCAG
TCCACAAGGTGCACCTGGAAACAATGATGCCCCTCAAGACCATCGGCCTGCACCACCATGGCTGCGTG
CCCCAGGCCATGGCACACACCCACCTGGGCGGCTACTTCTTCATCCAGTGCCGACAGGACAGCCCCGC
CTCTGCTGCCCGACAGCTGCTCGTTGACAGTGTCACAGACTCTGTGCTTGGCCCCAATGGTGATGTAA
CAGGCACCCCACACACATCCCCCGACGGGCGCTTCATAGTCAGTGCTGCAGCTCACAGCCCCTGGCTG
CACGTGCAGGAGATCACAGTGCGGGGCGAGATCCAGACCCTGTATGACCTGCAAATAAACTCGGGCAT
CTCAGACTTGGCCTTCCAGCGCTCCTTCACTGAAAGCAATCAATACAACATCTACGCGGCTCTGCACA
TGGAGCCGGACCTGCTGTTCCTGGAGCTGTCCACGGGGAAGGTGGGCATGCTGAAGAACTTAAAGGAG
CCACCCGCAGGGCCAGCTCAGCCCTGGGGGGGTACCCACAGAATCATGAGGGACAGTGGGCTGTTTGG
ACAGTACCTCCTCACACCAGCCCGAGAGTCACTGTTCCTCATCAATGGGAGACAAAACACGCTGCGGT
GTGAGGTGTCAGGTATAAAGGGGGGGACCACAGTGGTGTGGGTGGGTGAGGTACTCGAG
NOV17d,
268825987
Protein Sequence SEQ ID NO: 246 813 aa MW at 89900.2 kD
LEMKPGGFWLHLTLLGASLPAALGWMDPGTSRGPDVCVGESQAEEPRSFEVTRREGLSSHNELLASCG
KKFCSRGSRCVLSRKTGEPECLGKRITVIHSKDCFLKGDTCTMAGYARLKNVLLALQTRLQPLQEGDS
RQDPASQKRLLVESLFRDLDADGNGHLSSSELAQHVLKKQDLDEDLLGCSPGDLLRFDDYNSDSSLTL
REFYMAFQVVQLSLAPEDRVSVTTVTVGLSTVLTCAVHGDLRPPIIWKRNGLTLNFLDLEDINDFGED
DSLYITKVTTIHMGNYTCHASGHEQLFQTHVLQVNVPPVIRVYPESQAQEPGVAASLRCHAEGIPMPR
ITWLKNGVDVSTQMSKQLSLLANGSELHISSVRYEDTGAYTCIAKNEVGVDEDISSLFIEDSARKTLA
NILWREEGLSVGNMFYVFSDDGIIVIHPVDCEIQRHLKPTEKIFMSYEEICPQREKNATQPCQWVSAV
NVRNRYIYVAQPALSRVLVVDIQAQKVLQSIGVDPLPAKLSYDKSHDQVWVLSWCDVHKSRPSLQVIT
EASTGQSQHLIRTPFAGVDDFFIPPTNLIINHIRFGFIFNKSDPAVHKVDLETMMPLKTIGLHHHGCV
PQAMAHTHLGGYFFIQCRQDSPASAARQLLVDSVTDSVLGPNGDVTGTPHTSPDGRFIVSAAADSPWL
HVQEITVRGEIQTLYDLQINSGISDLAFQRSFTESNQYNIYAALHMEPDLLFLELSTGKVGMLKNLKE
PPAGPAQPWGGTHRIMRDSGLFGQYLLTPARESLFLINGRQNTLRCEVSGIKGGTTVVWVGEVLE
NOV17e,
268825997 SEQ ID NO: 247 2472 bp
DNA Sequence ORF Start: at 1 ORF Stop: end of sequence
CTCGAGTGGATGGACCCAGGAACCAGCAGAGGCCCGGATGTGCGTCTCGGGGAGTCACAGGCAGAGGA
GCCCAGAAGCTTTGAAGTCACAAGAAGAGAAGGGCTTTCCAGCCACAACGAGCTGCTGGCCTCCTGCG
GGAAGAAGTTCTGCAGCCGAGGGAGCCGGTGCGTGCTCAGCAGGAAGACAGGGGAGCCCGAATGCCAG
TGCCTGGAGGCATGCAGGCCCAGCTACGTCCCTGTGTGCGCCTCTGATGGGAGGTTTTATGAAAACCA
CTGTAAGCTCCACCGTCCTGCTTGCCTCCTGGGAAAGAGGATCACCGTCATCCACAGCAAGGACTGTT
TCCTCAAAGGTGACACGTGCACCATGGCCGGCTACGCCCGCTTGAAGAATGTCCTTCTGGCACTCCAG
ACCCGTCTGCAGCCACTCCAAGAAGGAGACAGCAGACAAGACCCTGCCTCCCAGAAGCGCCTCCTGGT
GCAATCTCTGTTCAGGGACTTAGATGCAGATGGCAATGGCCACCTCAGCAGCTCCGAACTGGCTCAGC
ATGTGCTGAAGAAGCAGGACCTGGATGAAGACTTACTTGGTTGCTCACCAGGTGACCTCCTCCGATTT
GACGATTACAACAGTGACAGCTCCCTGACCCTCCGCGAGTTCTACATGGCCTTCCAAGTGGTTCAGCT
CAGCCTCGCCCCCGAGGACAGGGTCAGTGTGACCACAGTCACCGTGGGGCTGAGCACAGTGCTGACCT
GCGCCGTCCATGGAGACCTGAGGCCACCAATCATCTGGAAGCGCAACGGGCTCACCCTGAACTTCCTG
GACTTGGAAGACATCAATGACTTTGGAGAGGATGATTCCCTGTACATCACCAAGGTGACCACCATCCA
CATGGGCAATTACACCTGCCATCCTTCCGGCCACGAGCAGCTGTTCCAGACCCACGTCCTGCAGGTGA
ATGTGCCGCCAGTCATCCGTGTCTATCCAGAGAGCCAGGCACAGGAGCCTGGAGTGGCAGCCAGCCTA
AGATGCCATGCTGAGGGCATTCCCATGCCCAGAATCACTTGGCTGAAAAACGGCGTGGATGTCTCAAC
TCAGATGTCCAAACAGCTCTCCCTTTTAGCCAATGGGAGCGAACTCCACATCAGCAGTGTTCGGTATG
AAGACACAGGGGCATACACCTGCATTGCCAAAAATGAAGTGGGTGTGGATGAAGATATCTCCTCGCTC
TTCATTGAAGACTCAGCTAGAAAGACCCTTGCAAACATCCTGTGGCGAGAGGAAGGCCTCAGCGTGGG
AAACATGTTCTATGTCTTCTCCGACGACGGTATCATCGTCATCCATCCTGTGGACTGTGAGATCCAGA
GGCACCTCAAACCCACGGAAAAGATTTTCATGAGCTATGAAGAAATCTGTCCTCAAAGAGAAAAAAAT
GCAACCCAGCCCTGCCAGTGGGTATCTGCAGTCAATGTCCGGAACCGGTACATCTATGTGGCCCAGCC
AGCACTGAGCAGAGTCCTTGTGGTCGACATCCAAGCCCAGAAAGTCCTACAGTCCATAGGTGTGGACC
CTCTGCCGGCTAAGCTGTCCTATGACAAGTCACATGACCAAGTGTGGGTCCTGAGCTGGGGGGACGTG
CACAAGTCCCGACCAAGTCTCCAGCTGATCACAGAAGCCAGCACCGGCCAGAGCCAGCACCTCATCCG
CACACCCTTTGCAGGAGTGGATGATTTCTTCATTCCCCCAACAAACCTCATCATCAACCACATCAGGT
TTGGCTTCATCTTCAACAAGTCTGATCCTACAGTCCACAAGGTGGACCTCGAAACAATGATGCCCCTC
AAGACCATCGGCCTGCACCACCATGGCTGCGTGCCCCAGGCCATGGCACACACCCACCTGGGCGGCTA
CTTCTTCATCCAGTGCCGACAGGACAGCCCCGCCTCTGCTGCCCGACAGCTGCTCGTTGACAGTGTCA
CAGACTCTGTGCTTGGCCCCAATGGTGATGTAACAGGCACCCCACACACATCCCCCGACGGGCGCTTC
ATAGTCAGTGCTGCAGCTGACAGCCCCTGGCTGCACGTGCAGGAGATCACAGTGCGGGGCGAGATCCA
GACCCTCTATGACCTGCAAATAAACTCGGGCATCTCAGACTTGGCCTTCCAGCGCTCCTTCACTGAAA
GCAATCAATACAACATCTACGCGGCTCTGCACACGGAGCCCGACCTGCTGTTCCTGGAGCTGTCCACG
GGGAACGTGGGCATGCTGAAGAACTTAAAGGAGCCACCCGCAGGGCCAGCTCAGCCCTGGGGGGGTAC
CCACAGAATCATGAGGGACAGTGGGCTGTTTGGACAGTACCTCCTCACACCAGCCCGAGAGTCACTGT
TCCTCATCAATGGGAGACAAAACACGCTGCGGTGTGAGCTGTCAGGTATAAAGGGGGGGACCACAGTG
GTGTGGGTGGGTGAGGTACTCGAG
NOV17e,
268825997
Protein Sequence SEQ ID NO: 248 824 aa MW at 91376.7 kD
LEWMDPGTSRGPDVGVGESQAEEPRSFEVTRREGLSSHNELLASCGKKFCSRGSRCVLSRKTGEPECQ
CLEACRPSYVPVCGSDGRFYENHCKLHRAACLLGKRITVIHSKDCFLKGDTCTMAGYARLKNVLLALQ
TRLQPLQEGDSRQDPASQKRLLVESLFRDLDADGNGHLSSSELAQHVLKKQDLDEDLLGCSPGDLLRF
DDYNSDSSLTLREFYMAFQVVQLSLAPEDRVSVTTVTVGLSTVLTCAVHGDLRPPIIWKRNGLTLNFL
DLEDINDFGEDDSLYITKVTTIHMGNYTCHASGHEQLFQTHVLQVNVPPVIRVYPESQAQEPGVAASL
RCHAEGIPMPRITWLKNGVDVSTQMSKQLSLLANGSELHISSVRYEDTGAYTCIAKNEVGVDEDISSL
FIEDSARKTLANILWREEGLSVGNMFYVFSDDGIIVIHPVDCEIQRHLKPTEKIFMSYEEICPQREKN
ATQPCQWVSAVNVRNRYIYVAQPALSRVLVVDIQAQKVLQSIGVDPLPAKLSYDKSHDQVWVLSWGDV
HKSRPSLQVITEASTGQSQHLIRTPFAGVDDFFIPPTNLIINHIRFGFIFNKSDPTVHKVDLETMMPL
KTIGLHHHGCVPQAMAHTHLGGYFFIQCRQDSPASAARQLLVDSVTDSVLGPNGDVTGTPHTSPDGRF
IVSAAADSPWLHVQEITVRGEIQTLYDLQINSGISDLAFQRSFTESNQYNIYAALHTEPDLLFLELST
GKVGMLKNLKEPPAGPAQPWGGTHRIMRDSGLFGQYLLTPARESLFLINGRQNTLRCEVSGIKGGTTV
VWVGEVLE
NOV17f,
275698334 SEQ ID NO: 249 2538 bp
DNA Sequence ORF Start: at 1 ORF Stop: end of sequence
CTCGAGATGAAACCAGGAGGCTTTTGGCTGCATCTCACACTGCTCGGAGCCTCCCTGCCGGCTGCGCT
GGGATGGATGCACCCAGGAACCAGCAGAGGCCCGGATGTGGGTGTGGGCGAGTCACAGGCAGAGGAGC
CCAGAAGCTTTGAAGTCACAAGAAGAGAAGGGCTTTCCAGCCACAACGAGCTGCTGGCCTCCTGCGGG
AAGAAGTTCTGCAGCCGAGGGAGCCGGTGCGTGCTCACCAGGAAGACAGGGGAGCCCGAATGCCAGTG
CCTGGAGGCATGCAGGCCCAGCTACGTGCCTGTGTGCGGCTCTGATGGGAGGTTTTATGAAAACCACT
CTAAGCTCCACCGTGCTGCTTGCCTCCTGGGAAAGAGGATCACCGTCATCCACAGCAAGGACTGTTTC
CTCAAAGGTGACACGTGCACCATGGCCGGCTACGCCCGCTTGAAGAATGTCCTTCTGGCACTCCAGAC
CCGTCTGCAGCCACTCCAAGAAGGACACAGCAGACAAGACCCTGCCTCCCAGAAGCGCCTCCTGGTGG
AATCTCTGTTCAGGGACTTAGATGCAGATGGCAATGGCCACCTCAGCAGCTCCGAACTGGCTCAGCAT
GTGCTGAAGAAGCAGGACCTGGATGAAGACTTACTTGGTTGCTCACCAGGTGACCTCCTCCGATTTGA
CGATTACAACAGTGACAGCTCCCTGACCCTCCGCGAGTTCTACATGGCCTTCCAAGTGGTTCAGCTCA
GCCTCGCCCCCGAGGACAGGGTCAGTGTGACCACAGTGACCCTGGGGCTGAGCACAGTGCTGACCTGC
GCCGTCCATGGAGACCTGAGGCCACCAATCATCTGCAAGCGCAACGGGCTCACCCTGAACTTCCTGGA
CTTGGAAGACATCAATGACTTTGGAGAGGATGATTCCCTGTACATCACCAAGGTGACCACCATCCACA
TGGGCAATTACACCTGCCATGCTTCCGGCCACGAGCAGCTGTTCCAGACCCACGTCCTGCAGGTGAAT
GTGCCGCCAGTCATCCGTGTCTATCCAGAGAGCCAGGCACAGGAGCCTGGAGTGGCAGCCAGCCTAAG
ATGCCATGCTGAGGGCATTCCCATGCCCAGAATCACTTGGCTGAAAAACGGCGTGGATGTCTCAACTC
AGATGTCCAAACAGCTCTCCCTTTTAGCCAATGGGAGCGAACTCCACATCAGCAGTGTTCGGTATGAA
GACACAGGGGCATACACCTGCATTGCCAAAAATGAAGTGGGTGTGGATGAAGATATCTCCTCGCTCTT
CATTGAAGACTCAGCTAGAAAGACCCTTGCAAACATCCTGTGGCCAGAGGAAGGCCTCAGCGTGGGAA
ACATGTTCTATGTCTTCTCCGACGACGGTATCATCGTCATCCATCCTGTGGACTGTGAGATCCAGAGG
CACCTCAAACCCACGGAAAAGATTTTCATGAGCTATGAAGAAATCTGTCCTCAAAGAGAAAAAAATGC
AACCCAGCCCTCCCAGTGGGTATCTGCAGTCAATGTCCGGAACCGGTACATCTATGTGGCCCAGCCAG
CACTGAGCAGAGTCCTTGTGGTCGACATCCAAGCCCAGAAAGTCCTACAGTCCATAGGTGTGGACCCT
CTGCCGGCTAACCTGTCCTATGACAAGTCACATGACCAAGTGTGGGTCCTGAGCTGGGGGGACGTGCA
CAAGTCCCGACCAAGTCTCCAGGTGATCACAGAAGCCAGCACCGGCCAGAGCCAGCACCTCATCCGCA
CACCCTTTGCAGGAGTGGATGATTTCTTCATTCCCCCAACAAACCTCATCATCAACCACATCAGGTTT
GGCTTCATCTTCAACAAGTCTGATCCTGCAGTCCACAAGGTGGACCTGGAAACAATGATGCCCCTCAA
GACCATCGGCCTGCACCACCATGGCTGCGTGCCCCAGGCCATGGCACACACCCACCTGGGCGGCTACT
TCTTCATCCAGTGCCGACAGGACAGCCCCGCCTCTGCTGCCCGACAGCTGCTCGTTGACAGTGTCACA
GACTCTGTGCTTGGCCCCAATGGTGATGTAACAGGCACCCCACACACATCCCCCGACGGGCCCTTCAT
AGTCAGTGCTGCAGCTGACAGCCCCTGGCTGCACGTGCAGGAGATCACAGTGCGGGGCGAGATCCAGA
CCCTGTATGACCTGCAAATAAACTCGGGCATCTCAGACTTGGCCTTCCAGCGCTCCTTCACTGAAAGC
AATCAATACAACATCTACGCGGCTCTGCACATGGAGCCGGACCTGCTGTTCCTCGAGCTGTCCACGGG
GAAGGTGGGCATGCTGAAGAACTTAAAGGAGCCACCCGCAGGGCCAGCTCAGCCCTGGGGGGGTACCC
ACAGAATCATGAGGGACAGTGGGCTGTTTGGACAGTACCTCCTCACACCAGCCCGAGAGTCACTGTTC
CTCATCAATGGGACACAAAACACGCTGCGGTGTGAGGTGTCAGGTATAAACGGGGGGACCACAGTGGT
GTGGGTGGGTGAGGTACTCGAG
NOV17f,
275698334
Protein Sequence SEQ ID NO: 250 846 aa MW at 93609.5 kD
LEMKPGGFWLHLTLLGASLPAALGWMDPGTSRGPDVGVGESQAEEPRSFEVTRREGLSSHNELLASCG
KKFCSRGSRCVLSRKTGEPECQCLEACRPSYVPVCGSDGRFYENHCKLHRAACLLGKRITVIHSAACF
LKGDTCTMAGYARLKNVLLALQTRLQPLQEGDSRQDPASQKRLLVESLFRDLDADGNGHLSSSELAQH
VLKKQDLDEDLLGCSPGDLLRFDDYNSDSSLTLREFYMAFQVVQLSLAPEDRVSVTTVTVGLSTVLTC
AVHGDLRPPIIWKRNGLTLNFLDLEDINDFGEDDSLYITKVTTIHMGNYTCHASGHEQLFQTHVLQVN
VPPVIRVYPESQAQEPGVAASLRCHAEGIPMPRITWLKNGVDVSTQMSKQLSLLANGSELHISSVRYE
DTGAYTCIAKNEVGVDEDISSLFIEDSARKTLANILWREEGLSVGNMFYVFSDDGIIVIHPVDCEIQR
HLKPTEKIFMSYEEICPQREKNATQPCQWVSAVNVRNRYIYVAQPALSRVLVVDIQAQKVLQSIGVDP
LPAKLSYDKSNDQVWVLSWGDVHKSRPSLQVITEASTGQSQHLIRTPFAGVDDFFIPPTNLIINHIRF
GFIFNKSDPAVHKVDLETMMPLKTIGLHHHGCVPQAMAHTHLGGYFFIQCRQDSPASAARQLLVDSVT
DSVLGPNGDVTGTPHTSPDGRFIVSAAADSPWLHVQEITVRCEIQTLYDLQINSGISDLAFQRSFTES
NQYNIYAALHMEPDLLFLELSTGKVGMLKNLKEPPAGPAQPWGGTHRIMRDSGLFGQYLLTPARESLF
LINGRQNTLRCEVSGIKGGTTVVWVGEVLE
NOV17g,
CG52643-04 SEQ ID NO: 251 2538 bp
DNA Sequence ORF Start: ATG at 7 ORF Stop: end of sequence
CTCGAGATGAAACCAGGAGGCTTTTGGCTGCATCTCACACTGCTCGGAGCCTCCCTGCCGGCTGCCCT
GGGATCGATGGACCCAGGAACCAGCAGAGGCCCGGATGTGGGTGTGGGGGAGTCACAGGCAGAGGAGC
CCAGAAGCTTTGAAGTCACAAGAAGAGAAGGGCTTTCCAGCCACAACGAGCTGCTGGCCTCCTGCGCG
AAGAAGTTCTGCAGCCGAGGGAGCCGGTGCGTGCTCAGCAGGAAGACAGGCGAGCCCGAATGCCAGTG
CCTCGAGGCATGCAGGCCCAGCTACGTGCCTGTGTGCGGCTCTGATGCCAGGTTTTATGAAAACCACT
CTAAGCTCCACCGTGCTGCTTGCCTCCTGGGAAAGAGGATCACCGTCATCCACAGCAAGGACTGTTTC
CTCAAAGGTGACACGTGCACCATGGCCGGCTACGCCCGCTTGAAGAATGTCCTTCTGGCACTCCAGAC
CCGTCTGCAGCCACTCCAAGAAGGAGACAGCAGACAAGACCCTGCCTCCCAGAAGCGCCTCCTGGTGG
AATCTCTGTTCAGGCACTTAGATGCAGATGGCAATGGCCACCTCAGCAGCTCCGAACTGGCTCAGCAT
GTGCTGAAGAAGCAGGACCTGGATGAAGACTTACTTCCTTGCTCACCAGGTGACCTCCTCCGATTTGA
CGATTACAACAGTGACAGCTCCCTGACCCTCCGCGAGTTCTACATGGCCTTCCAAGTGGTTCAGCTCA
GCCTCGCCCCCCAGGACAGGGTCAGTGTGACCACAGTGACCGTGGGGCTGAGCACAGTGCTGACCTGC
GCCGTCCATGGAGACCTGAGGCCACCAATCATCTGGAAGCGCAACGGGCTCACCCTGAACTTCCTGGA
CTTGGAAGACATCAATGACTTTGGAGAGCATGATTCCCTGTACATCACCAAGGTGACCACCATCCACA
TGGGCAATTACACCTGCCATGCTTCCGGCCACCAGCAGCTGTTCCAGACCCACGTCCTGCAGGTGAAT
GTGCCGCCAGTCATCCGTGTCTATCCAGAGAGCCAGGCACAGGAGCCTGGAGTGGCAGCCAGCCTAAG
ATGCCATGCTGAGGGCATTCCCATGCCCAGAATCACTTGGCTGAAAAACGGCGTGGATGTCTCAACTC
AGATGTCCAAACAGCTCTCCCTTTTAGCCAATGGGAGCGAACTCCACATCAGCAGTGTTCGGTATGAA
GACACAGGGGCATACACCTGCATTCCCAAAAATGAAGTGGGTCTCGATGAAGATATCTCCTCGCTCTT
CATTGAAGACTCAGCTAGAAAGACCCTTGCAAACATCCTGTGGCGAGAGGAAGGCCTCAGCGTGGGAA
ACATGTTCTATGTCTTCTCCGACGACGGTATCATCGTCATCCATCCTGTGGACTGTGAGATCCAGAGG
CACCTCAAACCCACGGAAAGATTTTCATGAGCTATGAAGAAATCTGTCCTCAAGAGAAAAAAAAATGC
AACCCAGCCCTGCCAGTGGGTATCTGCAGTCAATGTCCGGAACCGGTACATCTATGTGGCCCAGCCAG
CACTGAGCAGAGTCCTTGTGGTCGACATCCAAGCCCAGAAAGTCCTACAGTCCATAGGTGTGGACCCT
CTGCCGGCTAAGCTGTCCTATGACAAGTCACATGACCAAGTGTGGGTCCTGAGCTGGGGGGACCTGCA
CAAGTCCCGACCAAGTCTCCAGGTGATCACAGAAGCCAGCACCGGCCAGAGCCAGCACCTCATCCGCA
CACCCTTTGCAGGAGTGGATGATTTCTTCATTCCCCCAACAAACCTCATCATCAACCACATCAGGTTT
GGCTTCATCTTCACAGTCTGATCCTGCAGTCCACAAGGTCGACCTGGATAAACAATGATGCCCCTCAA
GACCATCGGCCTGCACCACCATGGCTGCGTGCCCCAGGCCATGGCACACACCCACCTGGGCGGCTACT
TCTTCATCCAGTGCCGACAGGACAGCCCCGCCTCTGCTGCCCGACAGCTGCTCGTTGACAGTGTCACA
GACTCTCTGCTTGGCCCCAATGGTGATGTAACAGGCACCCCACACACATCCCCCGACGGGCGCTTCAT
AGTCAGTGCTGCAGCTGACAGCCCCTGGCTGCACGTGCAGGAGATCACAGTGCGCGGCGAGATCCAGA
CCCTGTATGACCTGCAAATAAACTCGGGCATCTCAGACTTGGCCTTCCACCAGCTCCTTCACTGAAGC
AATCAATACAACATCTACGCGGCTCTGCACACGGAGCCGGACCTCCTGTTCCTGGAGCTGTCCACGGG
GAAGGTGGGCATGCTGAAGAACTTAAAGGAGCCACCCGCAGGGCCAGCTCAGCCCTGGGGGGGTACCC
ACAGAATCATGAGGGACAGTGGGCTCTTTGGACAGTACCTCCTCACACCAGCCCGAGAGTCACTGTTC
CTCATCAATGGCAGACAAAACACGCTGCGGTGTGAGGTGTCAGGTATAAAGGGGGGGACCACAGTGGT
GTGGGTGGGTGAGGTA
NOV17g,
CG52643-04
Protein Sequence SEQ ID NO: 252 842 aa MW at 93094.8 kD
MKPGGFWLHLTLLGASLPAALGWMDPGTSRGPDVGVGESQAEEPRSFEVTRREGLSSHNELLASCGKK
FCSRGSRCVLSRKTGEPECQCLEACRPSYVPVCGSDGRFYENHCKLHRAACLLGKRITVIHSKDCFLK
GDTCTMAGYARLKNVLLALQTRLQPLQEGDSRQDPASQKRLLVESLFRDLDADGNGHLSSSELAQHVL
KKQDLDEDLLGCSPGDLLRFDDYNSDSSLTLREFYMAFQVVQLSLAPEDRVSVTTVTVGLSTVLTCAV
HGDLRPPIIWKRNGLTLNFLDLEDINDFGEDDSLYITKVTTIHMGMYTCHASGHEQLFQTHAAQAAVP
PVIRVYPESQAQEPGVAASLRCHAEGIPMPRITWLKNGVDVSTQMSKQLSLLANGSELHISSVRYEDT
GAYTCIAKNEVGVDEDISSLFIEDSARKTLANILWREEGLSVGNMFYVFSDDGIIVIHPVDCEIQRHL
KPTEKIFMSYEEICPQREKNATQPCQWVSAVNVRNRYIYVAQPALSRVLVVDIQAQKVLQSIGVDPLP
AKLSYDKSHDQVWVLSWGDVHKSRPSLQVITEASTGQSQHLIRTPFAGVDDFFIPPTNLIINHIRFGF
IFNKSDPAVHKVDLETMMPLKTIGLHHHGCVPQAMAHTHLGGYFFIQCRQDSPASAARQLLVDSVTDS
VLGPNGDVTGTPHTSPDGRFIVSAAADSPWLHVQEITVRGEIQTLYDLQINSGISDLAFQRSFTESNQ
YNIYAALHTEPDLLFLELSTGKVGMLKNLKEPPAGPAQPWGGTHRIMRDSGLFGQYLLTPAAESLELI
NGRQNTLRCEVSGIKGGTTVVWVGEV
NOV17h,
301380586 SEQ ID NO: 253 2548 bp
DNA Sequence ORF Start: at 2 ORF Stop: end of sequence
CACCGGATCCACCATGAAACCAGGAGGCTTTTGGCTGCATCTCACACTGCTCGGAGCCTCCCTGCCGG
CTGCGCTGGGATGGATGGACCCAGGAACCAGCAGAGGCCCGGATGTGGGTGTGGGGGAGTCACAGGCA
GAGGAGCCCAGAAGCTTTGAAGTCACAAGAAGAGAAGGGCTTTCCAGCCACAACGAGCTGCTGGCCTC
CTGCGGGAAGAAGTTCTGCAGCCGAGGGAGCCGGTGCGTGCTCAGCAGGAAGACACGGGAGCCCGAAT
GCCAGTGCCTGGAGGCATGCAGGCCCAGCTACGTGCCTGTGTGCGGCTCTGATGCCAGGTTTTATGAA
AACCACTGTAAGCTCCACCGTGCTGCTTGCCTCCTGGGAAAGAGGATCACCGTCATCCACAGCAAGGA
CTCTTTCCTCAAAGGTGACACGTGCACCATGGCCGGCTACGCCCGCTTGAAGAATGTCCTTCTGGCAC
TCCAGACCCGTCTGCAGCCACTCCAAGAAGGAGACAGCAGACAAGACCCTGCCTCCCAGAAGCGCCTC
CTGGTGGAATCTCTGTTCAGGCACTTAGATCCAGATGGCAATGGCCACCTCAGCAGCTCCGAACTGGC
TCACCATGTGCTGAAGAAGCAGGACCTGGATGAACACTTACTTGGTTGCTCACCAGGTGACCTCCTCC
GATTTGACGATTACAACAGTCACAGCTCCCTGACCCTCCGCGAGTTCTACATGGCCTTCCAAGTGGTT
CAGCTCAGCCTCGCCCCCGAGGACAGGGTCAGTGTGACCACAGTGACCGTGGGGCTGAGCACAGTGCT
GACCTGCGCCGTCCATGCAGACCTGAGGCCACCAATCATCTGGAAGCGCAACGGGCTCACCCTGAACT
TCCTGGACTTGGAAGACATCAATGACTTTGGACAGGATGATTCCCTGTACATCACCAAGGTGACCACC
ATCCACATGGGCAATTACACCTGCCATGCTTCCGCCCACGAGCAGCTGTTCCAGACCCACGTCCTGCA
GGTGAATGTGCCGCCAGTCATCCGTGTCTATCCAGACAGCCAGGCACAGGAGCCTCGAGTGGCAGCCA
GCCTAAGATGCCATGCTGAGGGCATTCCCATGCCCAGAATCACTTGGCTGAAAAACGGCGTGGATGTC
TCAACTCAGATGTCCAAACAGCTCTCCCTTTTAGCCAATGGGAGCGAACTCCACATCAGCAGTGTTCG
GTATGAAGACACAGGGGCATACACCTGCATTGCCAAAAATGAAGTGGGTGTGGATGAAGATATCTCCT
CGCTCTTCATTGAAGACTCAGCTAGAAAGACCCTTGCAAACATCCTGTGGCGAGAGGAAGGCCTCAGC
GTGGGAAACATGTTCTATGTCTTCTCCGACGACGGTATCATCGTCATCCATCCTGTGGACTGTGAGAT
CCAGAGGCACCTCAAACCCACGGAAAAGATTTTCATCAGCTATGAAGAAATCTGTCCTCAAAGAGAAA
AAAATCCAACCCACCCCTGCCAGTGGGTATCTGCAGTCAATGTCCGGAACCGGTACATCTATGTGGCC
CAGCCAGCACTGAGCAGAGTCCTTGTGGTCGACATCCAAGCCCAGAAAGTCCTACAGTCCATAGGTGT
GGACCCTCTGCCGGCTAAGCTGTCCTATGACAAGTCACATGACCAAGTGTGGGTCCTGAGCTGGGGGG
ACGTGCACAAGTCCCGACCAAGTCTCCAGGTGATCACAGAAGCCAGCACCGGCCAGAGCCAGCACCTC
ATCCGCACACCCTTTGCAGGAGTGGATGATTTCTTCATTCCCCCAACAAACCTCATCATCAACCACAT
CAGGTTTGGCTTCATCTTCAACAAGTCTGATCCTGCAGTCCACAAGGTGGACCTGGAAACAATGATGC
CCCTCAAGACCATCGGCCTGCACCACCATGGCTGCGTGCCCCAGGCCATGGCACACACCCACCTGGGC
GGCTACTTCTTCATCCAGTGCCGACAGGACAGCCCCGCCTCTGCTGCCCGACAGCTGCTCGTTGACAG
TGTCACAGACTCTGTGCTTGGCCCCAATGGTCATCTAACAGGCACCCCACACACATCCCCCGACGGGC
GCTTCATAGTCAGTGCTGCAGCTGACAGCCCCTGGCTGCACGTGCAGGAGATCACAGTGCGGGGCGAG
ATCCAGACCCTGTATGACCTGCAAATAAACTCGGGCATCTCAGACTTGGCCTTCCAGCGCTCCTTCAC
TGAAAGCAATCAATACAACATCTACGCGGCTCTGCACACGGAGCCGGACCTGCTGTTCCTGGAGCTGT
CCACGGGGAAGGTGGGCATCCTCAACAACTTAAAGGAGCCACCCGCAGGGCCAGCTCAGCCCTGGGGG
GCTACCCACAGAATCATGAGGGACAGTGGGCTGTTTGGACAGTACCTCCTCACACCAGCCCGAGAGTC
ACTGTTCCTCATCAATGGGAGACAAAACACGCTGCGGTGTGAGGTGTCAGGTATAAAGGGGGGGACCA
CAGTGGTGTGGGTGGGTGAGGTAGAATTCGGC
NOV17h,
301380586
Protein Sequence SEQ ID NO: 254 849 aa MW at 93774.5 kD
TGSTMKPGGFWLHLTLLGASLPAALGWMDPGTSRGPDVGVGESQAEEPRSFEVTRREGLSSHNELLAS
CGKKFCSRGSRCVLSRKTGEPECQCLEACRPSYVPVCGSDGRFYENHCKLHRAACLLGKRITVIHSKD
CFLKGDTCTMAGYARLKNVLLALQTRLQPLQEGDSRQDPASQKRLLVESLFRDLDADGNGHLSSSELA
QHVLKKQDLDEDLLGCSPGDLLRFDDYNSDSSLTLREFYMAFQVVQLSLAPEDRVSVTTVTVGLSTVL
TCAVHGDLRPPIIWKRNGLTLNFLDLEDINDFGEDDSLYITKVTTIHMGNYTCHASGHEQLFQTHVLQ
VNVPPVIRVYPESQAQEPGVAASLRCHAEGIPMPRITWLKNGVDVSTQMSKQLSLLANGSELHISSVR
YEDTGAYTCIAKNEVGVDEDISSLFIEDSARKTLANILWREECLSVGNNFYVFSDDGIIVIHPVDCEI
QRHLKPTEKIFMSYEEICPQREKNATQPCQWVSAVNVRNRYIYVAQPALSRVLVVDIQAQKVLQSIGV
DPLPAKLSYDKSHDQVWVLSWGDVHKSRPSLQVITEASTGQSQHLIRTPFAGVDDFFIPPTNLIINHI
RFGFIFNKSDPAVHKVDLETMMPLKTIGLHHHGCVPQAMAHTHLGGYFFIQCRQDSPASAARQLLVDS
VTDSVLGPNGDVTGTPHTSPDGRFIVSAAADSPWLHVQEITVRGEIQTLYDLQINSGISDLAFQRSFT
ESNQYNIYAALHTEPDLLFLELSTGKVGMLKNLKEPPAGPAQPWGGTHRIMRDSGLFGQYLLTPARES
LELTNGRQNTLRCEVSGIKGGTTVVWVGEVEFG
NOV17i,
289087852 SEQ ID NO: 255 1842 bp
DNA Sequence ORF Start: at 1 ORF Stop: end of sequence
CTCGAGGGGCTGAGCACAGTGCTGACCTGCGCCGTCCATGGAGACCTGAGGCCACCAATCATCTGGAA
GCGCAACGGGCTCACCCTGAACTTCCTGGACTTGGAAGACATCAATGACTTTGGAGAGGATGATTCCC
TGTACATCACCAAGGTGACCACCATCCACATGGGCAATTACACCTGCCATGCTTCCGGCCACGAGCAG
CTGTTCCAGACCCACGTCCTGCAGGTGAATGTGCCGCCAGTCATCCGTGTCTATCCAGAGAGCCAGGC
ACAGGAGCCTGGAGTGGCAGCCAGCCTAAGATGCCATGCTGAGGGCATTCCCATGCCCAGAATCACTT
GGCTGAAAAACGGCGTGGATGTCTCAACTCAGATGTCCAAACAGCTCTCCCTTTTAGCCAATGGGAGC
GAACTCCACATCAGCAGTGTTCGGTATGAAGACACAGGGGCATACACCTGCATTGCCAAAAATGAAGT
GGGTGTGGATGAAGATATCTCCTCGCTCTTCATTGAAGACTCAGCTAGAAAGACCCTTGCAAACATCC
TGTGGCGAGAGGAAGATGCATCCACGTGGCCGGTTTCTTGTGTGTTCAATGCTGCGTGTGACCCTGCC
CAGGGGCCGACTGCTTGGAGGGCATGCCCATTCCATTTGCTCCTCCCAGGCCTCAGCGTGGGAAACAT
GTTCTATGTCTTCTCCGACGACGGTATCATCGTCATCCATCCTGTGGACTGTGAGATCCAGAGGCACC
TCAAACCCACGGAAAAGATTTTCATGAGCTATGAAGAAATCTGTCCTCAAAGAGAAAAAAATGCAACC
CAGCCCTGCCAGTGGGTATCTGCAGTCAATGTCCGGAACCGGTACATCTATGTGGCCCAGCCAGCACT
CAGCAGAGTCCTTGTGGTCCACATCCAAGCCCAGAAAGTCCTACAGTCCATAGGTGTGGACCCTCTGC
CGGCTAAGCTGTCCTATGGCAAGTCACATGACCAAGTGTGGGTCCTCAGCTGGGGGGACGTGCACAAG
TCCCGACCAAGTCTCCAGGTGATCACAGAAGCCAGCACCGGCCAGAGCCAGCACCTCATCCGCACACC
CTTTGCAGGAGTCGATGATTTCTTCATTCCCCCAACAAACCTCATCATCAACCACATCAGGTTTGGCT
TCATCTTCAACAAGTCTGATCCTGCAGTCCACAAGCTGGACCTGGAAACAATGATGCCCCTCAAGACC
ATCGGCCTCCACCACCATGGCTGCGTGCCCCAGGCCATGGCACACACCCACCTGGGCGGCTACTTCTT
CATCCAGTGCCGACAGGACAGCCCCGCCTCTCCTGCCCGACAGCTGCTCGTTGACAGTGTCACAGACT
CTGTGCTTGGCCCCAATGGCGATGTAACAGGCACCCCACACACATCCCCCGACGGGCGCTTCATAGTC
AGTGCTGCAGCTGACAGCCCCTGGCTGCACGTGCAGGAGATCACAGTGCGGGGCGAGATCCAGACCCT
GTATGACCTGCAAATAAACTCGGGCATCTCAGACTTGGCCTTCCAGCGCTCCTTCACTGAAAGCAATC
AATACAACATCTACGCGGCTCTGCACACGGAGCCGGACCTGCTGTTCCTGGAGCTGTCCACGCGGAAG
GTGGGCATGCTGAAGAACTTAAAGGAGCCACCCGCAGGGCCAGCTCAGCCCTGGGGGGGTACCCACAG
AATCATGAGGGACAGTGGGCTGTTTGGACAGTACCTCCTCACACCAGCCCGAGAGTCACTGTTCCTCA
TCAATGGGAGACAAAACACGCTGCGGTGTGAGGTGTCAGGTATAAAGGGGGGGACCACAGTGGTGTGG
CTCGAG
NOV17i,
289087852
Protein Sequence SEQ ID NO: 256 614 aa MW at 67956.6 kD
LEGLSTVLTCAVHGDLRPPIIWKRNGLTLNFLDLEDINDFGEDDSLYITKVTTIHMGNYTCHASGHEQ
LFQTHVLQVNVPPVIRVYPESQAQEPGVAASLRCHAEGIPMPRITWLKNGVDVSTQMSKQLSLLANGS
ELHISSVRYEDTGAYTCIAKNEVGVDEDISSLFIEDSARKTLANILWREEDASTWPVSCVFNAACDPA
QGPTAWRACPFHLLLPGLSVGNMFYVFSDDGIIVIHPVDCEIQRHLKPTEKIFMSYEEICPQREAAAT
QPCQWVSAVNVRNRYIYVAQPALSRVLVVDIQAQKVLQSIGVDPLPAKLSYGKSHDQVWVLSWGDVHK
SRPSLQVITEASTGQSQHLILRTPFAGVDDFFIPPTNLHNHIRFGFIFNKSDPAVHKVDLETMMPLKT
IGLHHHGCVPQAMAHTHLGGYFFIQCRQDSPASAARQLLVDSVTDSVLGPNGDVTGTPHTSPDGRFIV
SAAADSPWLHVQEITVRGEIQTLYDLQINSGISDLAFQRSFTESNQYNIYAALHTEPDLLFLELSTCK
VGMLKNLKEPPAGPAQPWGGTHRIMRDSGLFGQYLLTPARESLFLINGRQNTLRCEVSGIKGCTTVVW
LE
NOV17j,
289081920 SEQ ID NO: 257 1713 bp
DNA Sequence ORF Start: at 1 ORF Stop: end of sequence
CTCGAGGGGCTCAGCACAGTGCTGACCTGCGCCGTCCATGGAGACCTGAGGCCACCAATCATCTGGAA
GCGCAACGGGCTCACCCTGAACTTCCTGGACTTGGAAGACATCAATGACTTTGGAGAGGATGATTCCC
TGTACATCACCAAGGTGACCACCATCCACATGGGCAATTACACCTGCCATGCTTCCGGCCACGAGCAG
CTGTTCCAGACCCACGTCCTGCAGGTGAATGTGCCGCCAGTCATCCGTGTCTATCCAGAGAGCCAGGC
ACAGGAGCCTGGAGTGGCAGCCAGCCTAAGATGCCATGCTGAGGGCATTCCCATGCCCAGAATCACTT
GGCTGAAAAACGGCGTGGATGTCTCAACTCAGATGTCCAAACAGCTCTCCCTTTTAGCCAATGGGAGC
GAACTCCACATCAGCAGTGTTCGGTATGAAGACACAGGGGCATACACCTGCATTGCCAAAAATGAAGT
GGGTGTGGATGAAGATATCTCCTCGCTCTTCATTGAAGACTCAGCTAGAAAGACCCGCCTCAGCGTGG
GAAACATGTTCTATGTCTTCTCCGACGACGGTATCATCGTCATCCATCCTGTGGACTGTGAGATCCAG
AGGCACCTCAAACCCACGGAAAAGATTTTCATGAGCTATGAAGAAATCTGTCCTCAAAGAGAAAAAAA
TGCAACCCAGCCCTGCCAGTGGGTATCTGCAGTCAATGTCCGGAACCGGTACATCTATGTGGCCCAGC
CAGCACTGAGCAGAGTCCTTGTGGTCGACATCCAAGCCCAGAAAGTCCTACAGTCCATAGGTGTGGAC
CCTCTGCCGGCTAAGCTGTCCTATGACAAGTCACATGACCAAGTGTGGGTCCTGAGCTGGGGGGACGT
GCACAAGTCCCGACCAAGTCTCCAGGTGATCACAGAAGCCAGCACCGGCCAGAGCCAGCACCTCATCC
GCACACCCTTTGCAGGAGTGGATGATTTCTTCATTCCCCCAACAAACCTCATCATCAACCACATCAGG
TTTGGCTTCATCTTCAACAAGTCTGATCCTGCAGTCCACAAGGTGGACCTGGAAACAATGATGCCCCT
CAAGACCATCGGCCTGCACCACCATGGCTGCGTGCCCCAGGCCATGGCACACACCCACCTGGGCGGCT
ACTTCTTCATCCAGTGCCGACAGGACAGCCCCGCCTCTGCTGCCCGACAGCTGCTCGTTGACAGTGTC
ACAGACTCTGTGCTTGGCCCCAATGGTGATGTAACAGGCACCCCACACACATCCCCCGACGGGCGCTT
CATAGTCAGTGCTGCAGCTGACAGCCCCTGGCTGCACGTGCAGGAGATCACAGTGCGGGGCGAGATCC
AGACCCTGTATGACCTGCAAATAAACTCGGGCATCTCAGACTTGGCCTTCCAGCGCTCCTTCACTGAA
AGCAATCAATACAACATCTACGCGGCTCTGCACACGGAGCCGGACCTGCTGTTCCTGGAGCTGTCCAC
GGGGAAGGTGGGCATGCTGAAGAACTTAAAGGAGCCACCCGCAGGGCCAGCTCAGCCCTGGGGGGGTA
CCCACAGAATCATGAGGGACAGTGGGCTGTTTGGACAGTACCTCCTCACACCAGCCCGAGAGTCACTG
TTCCTCATCAATGGGAGACAAAACACGCTGCGGTGTGACGTGTCAGGTATAAGGGGGGGACCACAGT
AAGGTGTGGCTCGAG
NOV17j,
289081920
Protein Sequence SEQ ID NO: 258 571 aa MW at 63363.4 kD
LEGLSTVLTCAVHGDLRPPIIWKRNGLTLNFLDLEDINDFGEDDSLYITKVTTIHMGNYTCHASGHEQ
LFQTHVLQVNVPPVIRVYPESQAQEPGVAASLRCHAEGIPMPRITWLKNGVDVSTQMSKQLSLLANGS
ELHISSVRYEDTGAYTCIAKNEVGVDEDISSLFIEDSARKTRLSVGNMFYSFVDDGIIVIHPVDCEIQ
RHLKPTEKIFMSYEEICPQREKNATQPCQWVSAVNVRNRYILYVAQPALSRVLVDIQAQKVLQSIGVD
PLPAKLSYDKSHDQVWVLSWGDVHKSRPSLQVITEASTGQSQHLIPTPFAGVDDFFIPPTNLIINHIR
FGFIFNKSDPAVHKVDLETMMPLKTICLHHHGCVPQAMAHTHLGGYFFIQCRQDSPASAARQLLVDSV
TDSVLGPNGDVTGTPHTSPDGRFIVSAAADSPWLHVQEITVRGEIQTLYDLQINSGISDLAFQRSFTE
SNQYNIYAALHTEPDLLFLELSTGKVGMLKNLKEPPAGPAQPWGGTHRIMRDSGLFGQYLLTPARESL
FLINGRQNTLRCEVSGIKGGTTVVWLE
NOV17k,
289098038 SEQ ID NO: 259 1740 bp
DNA Sequence ORF Start: at 1 ORF Stop: end of sequence
CTCGAGGGGCTGAGCACAGTGCTGACCTGCGCCGTCCATGGAGACCTGAGGCCACCAATCATCTCGAA
GCGCAACCGCCTCACCCTGAACTTCCTGGACTTGGAAGACATCAATGACTTTGGAGAGGATGATTCCC
TGTACATCACCAACGTCACCACCATCCACATGGGCAATTACACCTGCCATGCTTCCGGCCACGAGCAG
CTGTTCCAGACCCACGTCCTGCACGTGAATGTGCCGCCAGTCATCCGTGTCTATCCAGAGAGCCAGGC
ACAGGAGCCTGGAGTGGCAGCCACCCTAAGATGCCATGCTGAGGGCATTCCCATGCCCAGAATCACTT
GGCTGAAAAACGGCGTGGATGTCTCAACTCAGATGTCCAAACAGCTCTCCCTTTTAGCCAATGGGAGC
GAACTCCACATCAGCAGTGTTCGGTATGAAGACACAGCGGCATACACCTGCATTGCCAAAAATGAAGT
GGGTGTGGATGAAGATATCTCCTCGCTCTTCATTGAAGACTCAGCTAGAAAGACCCTTGCAAACATCC
TGTGGCGAGAGGAAGGCCTCAGCGTGGGAAACATGTTCTATGTCTTCTCCGACGACGGTATCATCGTC
ATCCATCCTGTGGACTGTGAGATCCACAGGCACCTCAAACCCACGGAAAAGATTTTCATGAGCTATGA
AGAAATCTGTCCTCAAAGAGAAAAAAATGCAACCCAGCCCTGCCAGTGGGTATCTGCACTCAATGTCC
GGAACCGGTACATCTATGTGGCCCAGCCAGCACTGAGCAGAGTCCTTGTGGTCGACATCCAAGCCCAG
AAAGTCCTACAGTCCATAGGTGTGGACCCTCTGCCGGCTAAGCTGTCCTATGACAAGTCACATGACCA
AGTGTGGGTCCTGAGCTGGGGGGACGTGCACAAGTCCCGACCAAGTCTCCAGGTGATCACAGAAGCCA
GCACCGGCCAGAGCCAGCACCTCATCCGCACACCCTTTGCAGGAGTGGATGATTTCTTCATTCCCCCA
ACAAACCTCATCATCAACCACATCAGGTTTGGCTTCATCTTCAACAAGTCTGATCCTGCAGTCCACAA
GGTGGACCTGGAAACAATGATCCCCCTCAAGACCATCCGCCTGCACCACCATGCCTGCGTCCCCCACC
CCATCGCACACACCCACCTGCGCCGCTACTTCTTCATCCAGTCCCGACAGGACAGCCCCGCCTCTGCT
GCCCGACAGCTGCTCGTTGACAGTGTCACAGACTCTGTGCTTGGCCCCAATGGTGATGTAACAGGCAC
CCCACACACATCCCCCGACCGGCGCTTCATAGTCAGTGCTGCAGCTGACACCCCCTGGCTGCACGTGC
AGGAGATCACAGTGCGGGGCGAGATCCAGACCCTGTATGACCTGCAAATAAACTCGGGCATCTCAGAC
TTGGCCTTCCAGCGCTCCTTCACTGAAAGCAATCAATACAACATCTACGCGGCTCTGCACACGGAGCC
GGACCTGCTGTTCCTGGAGCTGTCCACGGGGAAGGTGGGCATGCTGAAGAACTTAAAGGAGCCACCCG
CAGGGCCAGCTCAGCCCTGGGGGGGTACCCACAGAATCATGAGGGACAGTGGGCTGTTTGGACAGTAC
CTCCTCACACCAGCCCGAGAGTCACTGTTCCTCATCAATGGGAGACAAAACACGCTGCGGTGTGAGGT
GTCAGGTATAAAGGGGGGGACCACAGTGGTGTGGCTCGAG
NOV17k,
289098038
Protein Sequence SEQ ID NO: 260 580 aa MW at 64389.6 kD
LEGLSTVLTCAVHGDLRPPIIWKRNGLTLNFLDLEDINDFGEDDSLYITKVTTIHMGNYTCHASGHEQ
LFQTHVLQVNVPPVIRVYPESQAQEPGVAASLRCHAEGIPMPRITWLKNGVDVSTQMSKQLSLLANGS
ELHISSVRYEDTGAYTCIAKNEVGVDEDISSLFIEDSARKTLANILWREEGLSVGNMFYVFSDDGIIV
IHPVDCEIQRHLKPTEKIFMSYEEICPQREKNATQPCQWVSAVNVRNRYIYVAQPALSRVLVVDIQAQ
KVLQSIGVDPLPAKLSYDKSHDQVWVLSWGDVHKSRPSLQVITEASTGQSQHLIRTPFAGVDDFFIPP
TNLIINHIRFIFIFNKSDPAVHKVDLETMMPLKTIGLHHHGCVPQAMAHTHLGGYFFTQCRQDSPASA
ARQLLVDSVTDSVLGPNGDVTGTPHTSPDGRFIVSAAADSPWLHVQEITVRGEIQTLYDLQINSGISD
LAFQRSFTESNQYNIYAALHTEPDLLFLELSTGKVGMLKNLKEPPAGPAQPWGGTHRIMRDSGLFGQY
LLTPARESLFLINGRQNTLRCEVSGIKGGTTVVWLE
NOV17l,
311060818 SEQ ID NO: 261 2508 bp
DNA Sequence ORF Start: at 13 ORF Stop: end of sequence
GCCAGGTGATGATATCTCAGATTCGCCTTCACCGGATCCTGGATCCACCCAGGAACCAGCAGAGGCCC
GGATGTGGGTGTGGGGGAGTCACAGGCAGAGGAGCCCAGAAGCTTTGAAGTCACAAGAAGAGAAGGGC
TTTCCAGCCACAACGAGCTGCTGGCCTCCTGCGGGAAGAAGTTCTGCAGCCGAGGGAGCCGGTGCGTG
CTCAGCAGGAAGACACCGGAGCCCCAATGCCAGTGCCTGGAGGCATGCAGGCCCAGCTACGTCCCTGT
GTGCGGCTCTGATGGGAGGTTTTATGAAAACCACTGTAAGCTCCACCGTGCTGCTTGCCTCCTGCGAA
AGAGGATCACCGTCATCCACAGCAACGACTGTTTCCTCAAAGGTGACACGTGCACCATGGCCGGCTAC
GCCCGCTTGAAGAATGTCCTTCTGGCACTCCAGACCCGTCTGCAGCCACTCCAAGAAGGAGACAGCAG
ACAAGACCCTGCCTCCCAGAAGCGCCTCCTGGTGGAATCTCTGTTCAGGGACTTAGATGCAGATGGCA
ATGGCCACCTCAGCACCTCCGAACTGGCTCAGCATGTGCTGAAGAAGCAGGACCTGGATGAAGACTTA
CTTGGTTCCTCACCACGTGACCTCCTCCCATTTGACGATTACAACAGTGACAGCTCCCTGACCCTCCG
CCAGTTCTACATGGCCTTCCAAGTGGTTCAGCTCAGCCTCGCCCCCGACAACAGCGTCAGTGTGACCA
CAGTGACCGTGGGGCTGAGCACAGTGCTCACCTGCGCCGTCCATGGAGACCTGAGGCCACCAATCATC
TCGAAGCGCAACGGGCTCACCCTGAACTTCCTGGACTTCGAAGACATCAATGACTTTGGAGAGGATGA
TTCCCTGTACATCACCAAGGTGACCACCATCCACATGGGCAATTACACCTGCCATGCTTCCGGCCACG
AGCAGCTGTTCCAGACCCACGTCCTGCAGGTGAATGTGCCGCCAGTCATCCGTGTCTATCCAGAGAGC
CAGGCACAGGAGCCTGGAGTGGCAGCCAGCCTAAGATGCCATGCTGAGCGCATTCCCATGCCCAGAAT
CACTTGGCTGAAAAACGGCGTGGATGTCTCAACTCAGATGTCCAAACAGCTCTCCCTTTTAGCCAATG
GGAGCGAACTCCACATCAGCAGTGTTCGGTATGAAGACACAGGGGCATACACCTGCATTGCCAAAAAT
GAAGTGGGTGTGGATGAAGATATCTCCTCGCTCTTCATTGAAGACTCAGCTAGAAAGACCCTTGCAAA
CATCCTGTGGCGAGAGGAAGGCCTCAGCGTGGGAAACATGTTCTATGTCTTCTCCGACGACGCTATCA
TCGTCATCCATCCTGTGGACTGTGAGATCCAGAGGCACCTCAAACCCACGGAAAAGATTTTCATGAGC
TATGAAGAAATCTGTCCTCAAAGAGAAAAAAATGCAACCCAGCCCTCCCAGTGGGTATCTGCAGTCAA
TGTCCGGAACCGGTACATCTATGTGGCCCAGCCAGCACTGAGCAGAGTCCTTGTGGTCGACATCCAAG
CCCAGAAAGTCCTACAGTCCATAGGTGTGGACCCTCTGCCGGCTAAGCTGTCCTATGACAAGTCACAT
GACCAAGTGTGGGTCCTGAGCTGGGGGGACGTGCACAAGTCCCGACCAAGTCTCCAGGTGATCACAGA
AGCCAGCACCGGCCAGAGCCAGCACCTCATCCGCACACCCTTTGCAGGAGTGGATGATTTCTTCATTC
CCCCAACAAACCTCATCATCAACCACATCAGGTTTGGCTTCATCTTCAACAAGTCTGATCCTGCAGTC
CACAAGGTGGACCTGGAAACAATGATGCCCCTCAAGACCATCGGCCTGCACCACCATGGCTGCGTGCC
CCAGGCCATGGCACACACCCACCTGGGCGGCTACTTCTTCATCCAGTGCCCACAGGACAGCCCCGCCT
CTGCTGCCCGACAGCTGCTCGTTGACAGTGTCACAGACTCTGTGCTTGGCCCCAATGGTGATGTAACA
GGCACCCCACACACATCCCCCGACGGGCGCTTCATAGTCAGTGCTGCAGCTGACAGCCCCTGGCTGCA
CGTGCAGGAGATCACAGTGCGGGGCGAGATCCAGACCCTGTATGACCTGCAAATAAACTCGGGCATCT
CAGACTTGGCCTTCCAGCGCTCCTTCACTGAAAGCAATCAATACAACATCTACGCGGCTCTGCACACG
GAGCCGGACCTGCTGTTCCTGGAGCTGTCCACGGGGAAGGTGGGCATGCTGAAGAACTTAAAGGAGCC
ACCCGCAGGGCCAGCTCAGCCCCGGGGGCGTACCCACAGAATCATGAGGGACAGTGGGCTGTTTGGAC
AGTACCTCCTCACACCAGCCCGAGAGTCACTGTTCCTCATCAATGGGAGACAAAACACCCTGCGGTGT
GAGGTGTCACGTATAAAGGGGGGGACCACAGTGGTGTGGGTGGGTGAGGTAGAATTCGCC
NOV17l,
311060818
Protein Sequence SEQ ID NO: 262 832 aa MW at 92208.6 kD
YLRFAFTGSWMDPGTSRGPDVGVGESQAEEPRSFEVTRREGLSSHNELLASCGKKFCSRGSRCVLSRK
TGEPECQCLEACRPSYVPVCGSDGRFYENHCKLHRAACLLGKRITVIHSKDCFLKGDTCTMAGYARLK
NVLLALQTRLQPLQEGDSRQDPASQKRLLVESLFRDLDADGNGHLSSSELAQHVLKKQDLDEDLLGCS
PGDLLRFDDYNSDSSLTLREFYMAFQVVQLSLAPEDRVSVTTVTVGLSTVLTCAVHGDLRPPIIWKRN
GLTLNFLDLEDINDFGEDDSLYITKVTTIHMGNYTCHASGHEQLFQTHVLQVNVPPVIRVYPESQAQE
PGVAASLRCHAECIPMPRITWLKNGVDVSTQMSKQLSLLANGSELHISSVRYEDTGAYTCIAKNEVGV
DEDISSLFIEDSARKTLANILWREEGLSVGNMFYVFSDDGILVIHPVDCEIQRHLKPTEKIFMSYEEI
CPQREKNATQPCQWVSAVNVRNRYIYVAQPALSRVLVVDIQAQKVLQSIGVDPLPAKLSYDKSHDQVW
VLSWGDVHKSRPSLQVITEASTGQSQHLIRTPFAGVDDFFIPPTNLIINHIRFGFIFNKSDPAVHKVD
LETMMPLKTIGLHHHGCVPQAMAHTHLGGYFFIQCRQDSPASAARQLLVDSVTDSVLGPNGDVTGTPH
TSPDGRFIVSAAADSPWLHVQEITVRGEIQTLYDLQINSGISDLAFQRSFTESNQYNIYAALHTEPDL
LFLELSTGKVGMLKNLKEPPAGPAQPRGGTHRIMRDSGLFGQYLLTPARESLFLINGRQNTLRCEVSC
IKGGTTVVWVGEVEFC
NOV17m,
311885703 SEQ ID NO: 263 2479 bp
DNA Sequence ORF Start: at 2 ORF Stop: end of sequence
CACCGGATCCTGGATGGACCCAGCAACCAGCAGAGGCCCGGATGTGGGTGTGGGGGAGTCACAGGCAG
AGGAGCCCAGAAGCTTTGAAGTCACAAGAAGAGAAGGGCTTTCCAGCCACAACGAGCTGCTGGCCTCC
TGCGGGAAGAAGTTCTGCAGCCGAGGGAGCCGGTGCGTGCTCAGCAGGAAGACAGGGGAGCCCGAATG
CCAGTGCCTCGAGGCATGCAGGCCCAGCTACGTGCCTGTGTGCGGCTCTGATGGGAGGTTTTATCAAA
ACCACTGTAAGCTCCACCGTGCTGCTTGCCTCCTGGGAAAGAGGATCACCGTCATCCACAGCAAGGAC
TGTTTCCTCAAAGGTGACACGTGCACCATGGCCGGCTACGCCCGCTTGAAGAATGTCCTTCTGGCACT
CCAGACCCGTCTGCAGCCACTCCAAGAAGGAGACAGCAGACAAGACCCTGCCTCCCAGAAGCGCCTCC
TGGTGGAATCTCTGTTCAGGGACTTAGATGCAGATGGCAATCCCCACCTCAGCAGCTCCGAACTGGCT
CAGCATGTGCTGAAGAAGCAGGACCTCGATGAACACTTACTTGGTTGCTCACCAGGTGACCTCCTCCG
ATTTGACGATTACAACAGTGACAGCTCCCTGACCCTCCGCGAGTTCTACATGGCCTTCCAAGTGGTTC
AGCTCAGCCTCGCCCCCGAGGACAGGGCCAGTGTGACCACAGTGACCGTCGCGCTGAGCACAGTGCTG
ACCTGCGCCGTCCATGGAGACCTGAGGCCACCAATCATCTGGAAGCGCAACGGGCTCACCCTGAACTT
CCTGGACTTGGAAGACATCAATGACTTTGCAGAGGATGATTCCCTGTACATCACCAAGGTGACCACCA
TCCACATCGGCAATTACACCTCCCATGCTTCCGGCCACGAGCAGCTGTTCCAGACCCACGTCCTGCAG
GTGAATGTGCCGCCAGTCATCCGTGTCTATCCAGAGAGCCAGGCACAGGAGCCTGGAGTGGCAGCCAG
CCTAAGATGCCATGCTGAGGGCATTCCCATGCCCAGAATCACTTGGCTGAAAAACGGCGTGGATGTCT
CAACTCAGATGTCCAAACAGCTCTCCCTTTTAGCCAATGGGAGCGAACTCCACATCAGCAGTGTTCGG
TATGAAGACACACGGGCATACACCTGCATTGCCAAAAATGAAGTGGGTGTGGATGAAGATATCTCCTC
GCTCTTCATTCAAGACTCAGCTAGAAACACCCTTGCAAACATCCTGTGGCGAGAGGAAGGCCTCAGCG
TGGGAAACATGTTCTATGTCTTCTCCGACCACGGTATCATCGTCATCCATCCTGTGGACTGTGAGATC
CAGAGGCACCTCAAACCCACGGAAAAGATTTTCATGAGCTATGAAGAAATCTGTCCTCAAAGAGAAAA
AAATGCAACCCAGCCCTGCCAGTGGGTATCTGCAGTCAATGTCCGGAACCGGTACATCTATGTGGCCC
AGCCAGCACTGAGCAGAGTCCTTGTGGTCGACATCCAAGCCCAGAAAGTCCTACAGTCCATAGGTGTG
GACCCTCTGCCGGCTAAGCTGTCCTATGACAAGTCACATGACCAAGTGTGGGTCCTGAGCTGGGGGGA
CGTGCACAAGTCCCGACCAAGTCTCCAGGTGATCACAGAAGCCAGCACCCGCCAGAGCCAGCACCTCA
TCCGCACACCCTTTGCAGGAGTGGATGATTTCTTCATTCCCCCAAAAACAACCTCATCATCCCACATC
AGGTTTGGCTTCATCTTCAACAAGTCTGATCCTGCAGTCCACAAAAAGGTGGACCTGGAACAGATGCC
CCTCAAGACCATCGGCCTGCACCACCATGGCTGCGTGCCCCAGGCCATGGCACACACCCACCTGGGCG
GCTACTTCTTCATCCAGTGCCGACAGGACAGCCCCGCCTCTGCTGCCCGACAGCTGCTCGTTGACAGT
GTCACAGACTCTGTGCTTGGCCCCAATGGTGATGTAACAGGCACCCCACACACATCCCCCGACCGGCG
CTTCATAGTCAGTGCTGCAGCTGACAGCCCCTGGCTGCACGTGCAGGAGATCACAGTGCCGGGCGAGA
TCCAGACCCTGTATGACCTGCAAATAAACTCGGGCATCTCAGACTTGGCCTTCCAGCGCTCCTTCACT
GAAAGCAATCAATACAACATCTACGCGGCTCTGCACACGGAGCCGGACCTGCTGTTCCTGGAGCTGTC
CACGGGGAAGGTGGGCATGCTGAAGAACTTAAAGGAGCCACCCGCAGGCCCAGCTCAGCCCTGGGGCG
GTACCCACAGAATCATGAGGGACAGTGGGCTGTTTGGACAGTACCTCCTCACACCAGCCCGAGAGTCA
CTGTTCCTCATCAATGGGAGACAAAACACGCTGCGGTGTGAGGTGTCAGGTATAAAGGGGGGGACCAC
AGTGGTGTGGGTCGGTGAGGTAGAATTCGGC
NOV17m,
311885703
Protein Sequence SEQ ID NO: 264 826 aa MW at 91412.7 kD
TGSWMDPGTSRGPDVGVCESQAEEPRSFEVTRREGLSSHNELLASCGKKFCSRGSRCVLSRKTGEPEC
QCLEACRPSYVPVCGSDGRFYENHCKLHRAACLLCKRITVIHSKLCFLKGDTCTMAGYARLKNVLLAL
QTRLQPLQEGDSRQDPASQKRLLVESLFRDLDADGNGHLSSSELAQHVLKKQDLDEDLLGCSPGDLLR
FDDYNSDSSLTLREFYMAFQVVQLSLAPEDRASVTTVTVGLSTVLTCAVHGDLRPPIIWKRNGLTLNF
LDLEDINDFGEDDSLYITKVTTIHMGNYTdHASGHEQLFQTHVLQVNVPPVIRVYPESQAQEPGVAAS
LRCHAEGIPMPRITWLKIAAGVDVSTQMSKQLSLLANGSELHISSVRYEDTGAYTCIAKNEVGVDISS
LFIEDSARKTLANILWREEGLSVGNMFYVFSDDGIIVIHPVDCEIQRHLKPTEKIFMSYEEICPQREK
NATQPCQWVSAVNVRNRYIYVAQPALSRVLVVDIQAQKVLQSIGVDPLPAKLSYDKSHDQVWVLSWGD
VHKSRPSLQVITEASTGQSQHLIRTPFAGVDDFFIPPTNLIINHIRFGFIFNKSDPAVHKVDLETMMP
LKTIGLHHHGCVPQANAHTHLCCYFFIQCRQDSPASAARQLLVDSVTDSVLGPNGDVTGTPHTSPDGR
FIVSAAADSPWLHVQEITVRGEIQTLYDLQINSGISDLAFQRSFTESNQYNIYAALHTEPDLLFLELS
TGKVGMLKNLKEPPAGPAQPWGGTHRIMRDSGLFGQYLLTPARESLFLINCRQNTLRCEVSGIKGGTT
VVWVGEVEFG
NOV17n,
CG52643-01 SEQ ID NO: 265 1689 bp
DNA Sequence ORF Start: ATG at 199 ORF Stop: TGA at 1147
TAGAATTCAGCGGCCGCTTAATTCTAGAACGAATGCCAGTGCCTGGAGGCATGCAGGCCCAGCTACGT
GCCTGTGTGCGGCTCTGATGGGAGGTTTTATGAAAACCACTGTAAGCTCCACCGTGCTGCTTGCCTCC
TGCGAAGAGGATCACCGTCATCCACAGCAAGGACTGTTTCCTCAAAAGGTGACACGTGCACCATGGCC
GGCTACGCCCGCTTGAAGAATGTCCTTCTGGCACTCCAGACCCGTCTGCAGCCACTCCAAGAAGGAGA
CAGCAGACAAGACCCTCCCTCCCAGAAGCGCCTCCTGGTGGAATCTCTGTTCAGGGACTTAGATGCAG
ATGGCATGGCCACCTCAGCAGCTCCGAACTGGCTCACCATGTGCTGAAGAAAGCAGGACCTGGATGAA
GACTTACTTGGTTGCTCACCAGGTGACCTCCTCCGATTTGACGATTACAACAGTGACAGCTCCCTGAC
CCTCCGCGAGTTCTACATGGCCTTCCAAGTGGTTCAGCTCAGCCTCGCCCCCGAGGACAGGGTCAGTG
TGACCACAGTGACCGTGGGGCTGACCACAGTGCTGACCTGCGCCGTCCATGGAGACCTAACGCCACCA
ATCATCTGGAAGCGCAACGGGCTCACCCTGAACTTCCTGGACTTGGAAGACATCAATGACTTTGGAGA
GGATGATTCCCTGTACATCACCAAGGTCACCACCATCCACATGGGCAATTACACCTGCCATGCTTCCG
GCCACGAGCAGCTCTTCCAGACCCACGTCCTGCAGGTGAATGTGCCGCCAGTCATCCGTGTCTATCCA
GAGAGCCAGGCACAGGAGCCTGGAGTGGCAGCCAGCCTAAGATGCCATGCTGAGGGCATTCCCATGCC
CAGAATCACTTGGCTGAAAAACGGCGTGGATGTCTCAACTCAGATGTCCAAACAGCTCTCCCTTTTAG
CCAATGGGAGCGAACTCCACATCAGCAGTGTTCGGTATGAAGACACAGGGGCATACACCTGCATTGCC
AAAAATGAAGTGGGTGTGGATGAAGATATCTCCTCGCTCTTCATTGAAGACTCAGCTAGAAAGACCCT
TGCAAACATCCTGTGGCGAGAGGAAGGTACCAACCTTCATTGTTTTGCGTCATGCCTGTGATCACGTG
TGTTTGGTTCTATGATGGGCCGTCTTTCCATGATCTGCCACCAGCTTTCCCACACAAAGCAGCCCTAT
GGGAGCAGGAAGTCAATGTCAAATTCAAGTGGCATATGCATTGAATCAAATTTAAAATGTACTCCTGT
CTTTAATGAGAAATTTTTAAATCCAAAGCTTTCATTAAAAGTGGCTTGTAACCTCTGCTGAAGCAGAA
CAGTTGGTAAGGGTTCCTGGTCAGATCTGGGCCTTAAACTTTTTTCCAGTAGCTGACTGGTGTTGGGT
TTAGTGTTTTGCCTATCTTGTGTGGTTTTAAAAAGACAAAACAAGTTGTAGATCTCTACTAGATAGTC
ACTGTACCTTAAATATGCTTTGATTGAGGAAAACCCGAGGAAAAAGCTGCCATGATTTCTGCCAATGT
ATATTTTTAAATGTATAGATGTTTAGAAACATATTTATCAAGCASATCTTTAGTAAGTTGAGCCATAT
GAAGTTGCCATTTTTGTGCATCAAAGTGGTCTAAGATTGACAATTTCATATGGCTGA
NOV17n,
CG52643-01
Protein Sequence SEQ ID NO: 266 316 aa MW at 35059.2 kD
MAGYARLKNVLLALQTRLQPLQEGDSRQDPASQKRLLVESLFRDLDADGNGHLSSSELAQHVLKKQDL
DEDLLGCSPGDLLRFDDYNSDSSLTLREFYMAFQVVQLSLAPEDRVSVTTVTVGLSTVLTCAVHGDLR
PPIIWKRNGLTLNFLDLEDINDFGEDDSLYITKVTTIHMGNYTCHASGHEQLFQTHVLQVNVPPVIRV
YPESQAQEPGVAASLRCHAEGIPMPRITWLKNGVDVSTQMSKQLSLLANGSELHISSVRYEDTGAYTC
IAKNEVGVDEDISSLFIEDSARKTLANILWREEGTAANCFASCL
NOV17o,
CG52643-03 SEQ ID NO: 267 1914 bp
DNA Sequence ORF Start: at 12 ORF Stop: end of sequence
TGGATGGACCCAGGAACCAGCAGAGGCCCGGATGTGGGTGTGGAGGAGTCACAGGCA
GAGGAGCCCAGAAGCTTTGAAGTCACAAGAAGAGAAGGGCTTTCCAGCCACAACGAGCTGCTGGCCTC
CTGCGGGAAGAAGTTCTGCAGCCGAGGGAGCCGGTGCGTGCTCAGCAGGAAGACAGGGGAGCCCCAAT
GCCAGTGCCTGGAGGCATGCAGGCCCAGCTACGTGCCTGTGTGCGGCTCTGATGGGAGGTTTTATGAA
AACCACTGTAAGCTCCACCGTGCTGCTTGCCTCCTGGGAAAGAGGATCGCCGTCATCCACAGCAAGGA
CTGTTTCCTCAAAGGTGACACGTGCACCATGGGCGGCTACGCCCGCTTGAAGAATGTCCTTCTGGCAC
TCCAGACCCGTCTGCAGCCACTCCAAGAAGGAGACAGCAGACAAGACCCTGCCTCCCAGAAGCGCCTC
CTGGTGGAATCTCTGTTCAGGCACTTAGATGCAGATGGCAATGGCCACCTCAGCAGCTCCGAACTGGC
TCAGCATGTGCTGAAGAAGCAGGACCTGGATGAAGACTTACTTGGTTGCTCACCAGGTGACCTCCTCC
GATTTGACGATTACAACAGTGACAGCTCCCTGACCCTCCGCGAGTTCTACATGGCCTTCCAAGTGGTT
CACCTCAGCCTCGCCCCCGAGGACAGGGTCAGTGTGACCACAGTGACCGTGGGCCTGAGCACAGTGCC
GACCTGCGCCGTCCATGGAGACCTGAGGCCACCAATCATCTGGAACCGCAACGGGCTCACCCTGAACT
TCCTGGACTTGGAAGACATCAATGGGAGACAAAACACGCTGCGGTGTGAGGTGTCAGGTATAAAGGGG
CGGACCACAGTGGTGTGGGTGGGTGACGTA
NOV17o,
CG52643-03
Protein Sequence SEQ ID NO: 268 301 aa MW at 33218.2 kD
WMDPGTSRGPDVGVEESQAEEPRSFEVTRREGLSSHNELLASCGKKFCSRGSRCAASRKTGEPECQCL
EACRPSYVPVCGSDGRFYENHCKLHRAACLLGKRIAVIHSKDCFLKGDTCTMGGYARLKNVLLALQTR
LQPLQEGDSRQDPASQKRLLVESLFRDLDADGNGHLSSSELAQHVLKKQDLDEDLLGCSPGDLLRFDD
YNSDSSLTLREFYMAFQVVQLSLAPEDRVSVTTVTVGLSTVPTCAVHGDLRPPIIWKRNGLTLNFLDL
EDINGRQNTLRCEVSGIKGGTTVVWVGEV
NOV17p,
CG52643-05 SEQ ID NO: 269 2548 bp
DNA Sequence ORF Start: ATG at 14 ORF Stop: end of sequence
CACCGGATCCACCATGAAACCAGGAGGCTTTTGGCTGCATCTCACACTGCTCGGAGCCTCCCTGCCGG
CTGCGCTGGGATGGATGGACCCAGGAACCAGCAGAGGCCCGGATGTGGGTGTGGGGGAGTCACAGGCA
GAGGAGCCCAGAAGCTTTGAAGTCACAAGAAGAGAAGGGCTTTCCAGCCACAACGAGCTGCTGGCCTC
CTGCGGGAAGAAGTTCTGCAGCCGAGGGAGCCGGTGCGTGCTCAGCAGGAAGACAGGGGAGCCCGAAT
GCCAGTGCCTGGAGGCATGCAGGCCCAGCTACGTGCCTGTGTGCGGCTCTGATGGGAGGTTTTATGAA
AACCACTGTAAGCTCCACCGTCCTGCTTCCCTCCTGGGAAAGAGGATCACCGTCATCCACAGCAAAGA
CTGTTTCCTCAAAGGTGACACGTGCACCATGGCCGGCTACGCCCGCTTGAAGAATGTCCTTCTGGCAC
TCCAGACCCGTCTGCAGCCACTCCAAGAAGGAGACAGCAGACAAGACCCTGCCTCCCAGAAGCGCCTC
CTGGTGGAATCTCTGTTCAGGGACTTAGATGCAGATGGCAATGGCCACCTCAGCAGCTCCGAACTCGC
TCAGCATGTGCTGAAGAACCAGGACCTGGATGAAGACTTACTTGGTTGCTCACCAGGTGACCTCCTCC
CATTTGACGATTACAACAGTGACAGCTCCCTGACCCTCCGCGAGTTCTACATGGCCTTCCAAGTGGTT
CAGCTCAGCCTCGCCCCCGAGGACAGGGTCAGTGTGACCACAGTGACCGTGGGGCTGAGCACAGTGCT
GACCTGCGCCGTCCATGGAGACCTGAGGCCACCAATCATCTGGAAGCGCAACGGGCTCACCCTGAACT
TCCTGGACTTGGAAGACATCAATGACTTTGGAGAGGATGATTCCCTGTACATCACCAAGGTGACCACC
ATCCACATGGGCAATTACACCTGCCATGCTTCCGGCCACGAGCAGCTGTTCCACACCCACCTCCTGCA
GGTGAATGTGCCGCCAGTCATCCGTGTCTATCCAGAGAGCCAGGCACAGGAGCCTGGAGTGGCAGCCA
GCCTAAGATGCCATGCTGAGGGCATTCCCATGCCCAGAATCACTTGGCTGAAAAACGGCGTGGATGTC
TCAACTCAGATGTCCAAACAGCTCTCCCTTTTAGCCAATGGGAGCGAACTCCACATCAGCAGTGTTCC
GTATGAAGACACAGGGGCATACACCTGCATTGCCAAAAATGAAGTGGGTGTGGATGAAGATATCTCCT
CGCTCTTCATTGAAGACTCAGCTAGAAGACCCTTGCAAAACATCCTGTGGCGAGAGGAAGGCCTCAGC
GTGGGAAACATGTTCTATGTCTTCTCCGACGACGCTATCATCGTCATCCATCCTGTGGACTGTGAGAT
CCAGAGGCACCTCAAACCCACGGAAAAGATTTTCATGAGCTATGAAGAAATCTGTCCTCAAAGAGAAA
AAAATGCAACCCAGCCCTGCCAGTGGGTATCTGCAGTCAATGTCCGGAACCGGTACATCTATGTGGCC
CAGCCAGCACTGAGCAGAGTCCTTGTGGTCGACATCCAAGCCCAGAAAGTCCTACAGTCCATAGGTGT
GGACCCTCTGCCGGCTAAGCTGTCCTATGACAAGTCACATGACCAAGTGTGGGTCCTGAGCTGGGGGG
ACGTGCACAAGTCCCGACCAAGTCTCCAGGTGATCACAGAAGCCAGCACCGGCCAGAGCCAGCACCTC
ATCCGCACACCCTTTGCAGGAGTGGATGATTTCTTCATTCCCCCAAACAACCTCATCATCAACCACAT
CAGGTTTGGCTTCATCTTCAACAGTCTGATCCTGCAGTCCACAAGGTGGACCTGGAACAAAATGATGC
CCCTCAAGACCATCGGCCTGCACCACCATGGCTGCGTGCCCCAGGCCATGGCACACACCCACCTGGGC
GGCTACTTCTTCATCCAGTGCCGACAGGACAGCCCCGCCTCTCCTGCCCGACAGCTGCTCGTTGACAG
TGTCACAGACTCTGTGCTTGGCCCCAATGGTGATGTAACAGGCACCCCACACACATCCCCCGACGGCC
GCTTCATAGTCAGTGCTGCAGCTGACAGCCCCTGGCTGCACGTGCACGAGATCACAGTGCGGGGCGAG
ATCCAGACCCTGTATGACCTGCAAATAAACTCGGGCATCTCAGACTTGGCCTTCCAGCGCTCCTTCAC
TGAAAGCAATCAATACAACATCTACGCGGCTCTGCACACGGAGCCGGACCTGCTGTTCCTGGAGCTGT
CCACCGGGAAGGTGGGCATGCTGAAGAACTTAAAGGAGCCACCCGCAGGGCCAGCTCACCCCTGGGGG
GGTACCCACAGAATCATGAGGGACAGTGGGCTGTTTGCACAGTACCTCCTCACACCAGCCCGAGAGTC
ACTGTTCCTCATCAATGGGAGACAAAACACGCTGCGGTGTGAGGTGTCAGGTATAAAGGGGGGGACCA
CAGTGGTGTGGGTGGGTGAGGTA
NOV17p,
CG52643-05
Protein Sequence SEQ ID NO: 270 842 aa MW at 93094.8 kD
MKPGGFWLHLTLLGASLPAALGWMDPGTSRGPDVGVGESQAEEPRSFEVTRREGLSSHNELLASCGKK
FCSRGSRCVLSRKTGEPECQCLEACRPSYVPVCGSDGRFYENHCKLHRAACLLGKRITVIHSKDCFLK
GDTCTMAGYARLKNVLLALQTRLQPLQEGDSRQDPASQKRLLVESLFRDLDADGNGHLSSSELAQHVL
KKQDLDEDLLGCSPCDLLRFDDYNSDSSLTLREFYMAFQVVQLSLAPEDRVSVTTVTVGLSTVLTCAV
HGDLRPPIIWKRNGLTLNFLDLEDINDFGEDDSLYITKVTTIHMGNYTCHASGHEQLFQTHVLQVNVP
PVIRVYPESQAQEPGVAASLRCHAEGIPMPRITWLKNGVDVSTQMSKQLSLLANGSELIHSSVRYEDT
GAYTCIAKNEVGVDEDISSLFIEDSARKTLANILWREEGLSVGNMFYVFSDDGIIVIHPVDCEIQRHL
KPTEKIFMSYEEICPQREKNATQPCQWVSAVNVRNRYIYVAQPALSRVLVVDIQAQKVLQSIGVDPLP
AKLSYDKSHDQVWVLSWGDVHKSRPSLQVITEASTGQSQHLIRTPFAVGVDDFFIPPTNLHNHIRFGF
IFNKSDPAVIHKVDLETMMPLKTIGLHHHGCVPQAMAHTHLGGYFFIQCRQDSPASAARQLVDSVTDS
VLGPNGDVTGTPHTSPDCRFIVSAAADSPWLHVQEITVRGEIQTLYDLQINSGISDLAFQRSFTESNQ
YNIYAALHTEPDLLFLELSTGKVCMLKNLKEPPAGPAQPWGGTHRIMRDSGLFGQYLLTPARESLFLI
NGRQNTLRCEVSGIKGGTTVVWVGEV
NOV17q,
CG52643-06 SEQ ID NO: 271 2460 bp
DNA Sequence ORF Start: at 1 ORF Stop: end of sequence
TGGATGGACCCAGGAACCAGCAGAGGCCC
GGATGTGGGTGTGGGGGAGTCACAGGCAGAGGAGCCCAGAAGCTTTGAAGTCACAAGAAGAGAAGGGC
TTTCCAGCCACAACGAGCTGCTGGCCTCCTGCGGGAAGAAGTTCTGCAGCCGAGCGAGCCGGTGCGTG
CTCAGCAGGAAGACAGGGGAGCCCGAATGCCAGTGCCTGGAGGCATGCAGGCCCAGCTACGTGCCTGT
GTGCGGCTCTGATGGGAGGTTTTATGAAAACCACTGTAAGCTCCACCGTCCTGCTTGCCTCCTGGGAA
AGAGGATCACCGTCATCCACAGCAAGGACTGTTTCCTCAAAGGTGACACGTGCACCATGGCCGCCTAC
GCCCGCTTGAAGAATCTCCTTCTGGCACTCCAGACCCGTCTGCAGCCACTCCAAGAAGGAGACAGCAG
ACAAGACCCTGCCTCCCAGAAGCGCCTCCTGGTGGAATCTCTGTTCAGGGACTTAGATGCAGATGGCA
ATGGCCACCTCAGCAGCTCCGAACTGGCTCAGCATGTGCTGAAGAAGCAGGACCTGGATGAAGACTTA
CTTGGTTGCTCACCAGGTGACCTCCTCCGATTTGACGATTACAACAGTGACAGCTCCCTGACCCTCCG
CGAGTTCTACATGGCCTTCCAAGTGGTTCAGCTCAGCCTCGCCCCCGAGGACAGGGTCAGTGTGACCA
CAGTGACCGTGGGGCTGAGCACAGTGCTGACCTGCGCCGTCCATCGAGACCTGAGGCCACCAATCATC
TGGAAGCGCAACGCGCTCACCCTGAACTTCCTGGACTTGGAAGACATCAATGACTTTGGAGAGGATGA
TTCCCTGTACATCACCAAGGTGACCACCATCCACATGGGCAATTACACCTGCCATGCTTCCGGCCACG
AGCAGCTGTTCCAGACCCACGTCCTGCAGGTGAATGTGCCGCCAGTCATCCGTGTCTATCCAGAGAGC
CAGGCACAGGAGCCTGGAGTGGCAGCCAGCCTAAGATGCCATGCTGAGGGCATTCCCATGCCCAGAAT
CACTTGGCTGAAAAACGGCGTGGATGTCTCAACTCAGATGTCCAAACAGCTCTCCCTTTTAGCCAATG
GGAGCGAACTCCACATCAGCAGTGTTCGGTATGAAGACACAGGuGCATACACCTGCATTGCCAAAAAT
GAAGTGGGTGTGGATGAAGATATCTCCTCGCTCTTCATTGAAGACTCAGCTAGAAAGACCCTTGCAAA
CATCCTGTGGCCAGAGGAAGGCCTCAGCGTGGGAAACATGTTCTATGTCTTCTCCGACGACGGTATCA
TCGTCATCCATCCTGTGGACTGTGAGATCCAGAGGCACCTCAAACCCACGGAAAAGATTTTCATGAGC
TATGAAGAAATCTGTCCTCAAAGAGAAAAAAATGCAACCCAGCCCTGCCAGTGGGTATCTGCAGTCAA
TGTCCGGAACCGGTACATCTATGTGGCCCAGCCAGCACTGAGCAGAGTCCTTGTGGTCGACATCCAAG
CCCAGAAAGTCCTACAGTCCATAGGTGTGGACCCTCTGCCGGCTAAGCTGTCCTATGACAAGTCACAT
GACCAAGTGTGGGTCCTGACCTGGGGGGACGTGCACAAGTCCCGACCAAGTCTCCAGGTGATCACAGA
AGCCAGCACCGGCCAGAGCCAGCACCTCATCCGCACACCCTTTGCAGGAGTGGATGATTTCTTCATTC
CCCCAACAAACCTCATCATCAACCACATCAGGTTTGGCTTCATCTTCAACAAGTCTGATCCTGCAGTC
CACAAGGTGGACCTGGAAACAATGATGCCCCTCAAGACCATCGGCCTGCACCACCATGGCTGCGTGCC
CCAGGCCATGGCACACACCCACCTGGGCGGCTACTTCTTCATCCAGTGCCGACAGCACAGCCCCGCCT
CTGCTGCCCGACAGCTGCTCGTTGACAGTGTCACAGACTCTGTGCTTGGCCCCAATGGTGATGTAACA
GGCACCCCACACACATCCCCCGACGGGCGCTTCATAGTCAGTGCTGCAGCTGACAGCCCCTGGCTGCA
CGTGCAGGAGATCACAGTGCGGGGCGAGATCCAGACCCTGTATGACCTGCAAATAAACTCGGGCATCT
CAGACTTGGCCTTCCAGCGCTCCTTCACTGAAAGCAATCAATACAACATCTACGCGGCTCTGCACACG
GAGCCGGACCTGCTGTTCCTGGAGCTGTCCACGGGGAAGGTGGGCATGCTGAAGAACTTAAAGGAGCC
ACCCGCAGGCCCAGCTCAGCCCCGGGGGGGTACCCACAGAATCATGAGGGACAGTGGGCTGTTTGGAC
AGTACCTCCTCACACCAGCCCGAGAGTCACTGTTCCTCATCAATGGGAGACAAAACACGCTGCGGTGT
GAGGTGTCAGGTATAAAGGGGGGGACCACAGTGGTGTGGGTGGGTGAGGTA
NOV17q,
CG52643-06
Protein Sequence SEQ ID NO: 272 820 aa MW at 90832.1 kD
WMDPGTSRGPDVGVGESQAEEPRSFEVTRREGLSSHNELLASCGKKFCSRGSRCVLSRKTGEPECQCL
EACRPSYVPVCGSDGRFYENHCKLNAAACLLGKRITVIHSAACFLKCDTCTAAGYARLAAVLLALQTR
LQPLQEGDSRQDPASQKRLLVESLFRDLDADGNGHLSSSELAQHVLKKQDLDEDLLGCSPGDLLRFDD
YNSDSSLTLREFYMAFQVVQLSLAPEDRVSVTTVTVGLSTVLTCAAAGDLRPPIIWKRNGLTLNFLDL
EDTNDFGEDDSLYITKVTTIHMGNYTCHASGHEQLFQTHVLQVNVPPVIRVYPESQAQEPGVAASLRC
HAEGIPMPRITWLKNGVDVSTQMSKQLSLLANGSELHISSVRYEDTGAYTCIAKNEVGVDEDISSLFI
EDSARKTLANTLWREEGLSVGNMFYVFSDDGIIVIHPVDCEIQRHLKPTEKIFMSYEEICPQREKNAT
QPCQWVSAVNVRNRYIYVAQPALSRVLVVDIQAQKVLQSIGVDPLPAKLSYDKSHDQVWVLSWGDVHK
SRPSLQVITEASTGQSQHLIRTPFAGVDDFFIPPTNLIINHIRFGFIFNKSDPAVHKVDLETMMPLKT
IGLHHHGCVPQANAHTHLGGYFFIQCRQDSPASAARQLLVDSVTDSVLGPNGDVTGTPHTSPDGRFIV
SAAADSPWLHVQEITVRGEIQTLYDLQINSGISDLAFQRSFTESNQYNTYAALHTEPDLLFLELSTGK
VCMLKNLKEPPAGPAQPRGGTHRIMRDSGLFGQYLLTPARESLFLINGRQNTLRCEVSGIKGGTTVVW
VGEV
NOV17r,
13382322 SNP
for CG52643-02 SEQ ID NO: 273 5573 bp SNP: 517 G/A
DNA Sequence ORF Start: ATG at 408 ORF Stop: TGA at 2934
GGAGAGGGCTGCATTCCTGTTGCTCACTGACCTTCTTTTATGCTGGCCTTTGGTTCAGAATGGCACATCA
TTCCTCGTTTTTGGCCCTCCAGCTGAACACCTGTTCTCTGTGGCACTGACTCCTCTTTCCATAGGGACAT
CATACAACAGTCGCCTTTATCTGAGGTTGTGCAAAGAGGGATGGAGGAGAAAACAATGGAGAATCCCTGG
CAGATTTCCCCAGGACGAGAGAAGGATATCCAATTGCTCATCAGGGAAGGTGCTAGGTCTCCCACCCAGA
CGCCCTCAGAGGCCGGTGTCAAGTCTCCCTCACCTCTGTGATGTGAAGTCAGCTCGTTCATGACCTGGGC
AGGCAGAGGGTCAGAGGGGCAGATGGAGCACTCCTCGCCTGATGAAGACTCATCAAAATGAAACCAGGAG
GCTTTTGGCTGCATCTCACACTGCTCGGACCCTCCCTGCCGGCTGCGCTGGGATGGATGGACCCAGGAAC
CAGCAGAGGCCCGGATGTGGGTGTGGAGGAGTCACAGGCAGAGGAGCCCACAAGCTTTGAACTCACAAGA
AGAGAAGGGCTTTCCAGCCACAACGACCTGCTGGCCTCCTGCGGGAAGAAGTTCTGCAGCCGAGGGAGCC
GGTGCGTGCTCAGCAGGAAGACAGGGGAGCCCGAATGCCAGTGCCTGGAGGCATGCAGGCCCAGCTACGT
GCCTGTGTGCGGCTCTGATGGGAGGTTTTATGAAAACCACTGTAAGCTCCACCGTGCTGCTTGCCTCCTG
GGAAAGAGGATCACCGTCATCCACAGCAAGGACTGTTTCCTCAAAGGTGACACGTGCACCATGGCCGGCT
ACGCCCGCTTGAAGAATGTCCTTCTGGCACTCCAGACCCGTCTGCAGCCACTCCAAGAAGGAGACAGCAG
ACAAGACCCTGCCTCCCAGAAGCGCCTCCTGGTGGAATCTCTGTTCAGGGACTTAGATGCAGATGGCAAT
GGCCACCTCACCAGCTCCGAACTGGCTCAGCATGTGCTGAAGAAGCAGGACCTGGATGAAGACTTACTTG
GTTGCTCACCAGGTGACCTCCTCCGATTTGACGATTACAACAGTGACAGCTCCCTGACCCTCCGCGAGTT
CTACATGGCCTTCCAAGTGGTTCAGCTCAGCCTCGCCCCCGAGGACAGGGTCAGTGTGACCACAGTGACC
GTGGGGCTGAGCACAGTGCTGACCTGCGCCGTCCATGGAGACCTGAGGCCACCAATCATCTGGAAGCGCA
ACGGGCTCACCCTGAACTTCCTGGACTTGGAAGACATCAATGACTTTGGAGAGGATGATTCCCTGTACAT
CACCAAGGTGACCACCATCCACATGGGCAATTACACCTGCCATGCTTCCGGCCACGAGCAGCTGTTCCAG
ACCCACGTCCTGCAGGTGAATGTGCCGCCAGTCATCCGTGTCTATCCAGAGAGCCAGGCACAGCAGCCTG
GAGTGGCAGCCAGCCTAAGATGCCATGCTGAGGGCATTCCCATGCCCAGAATCACTTGGCTGAAAAACGG
CGTGGATGTCTCAACTCAGATGTCCAAACAGCTCTCCCTTTTAGCCAATGGGAGCGAACTCCACATCAGC
AGTGTTCGGTATGAAGACACAGGGGCATACACCTGCATTGCCAAAAATGAAGTGGGTGTGGATGAAGATA
TCTCCTCGCTCTTCATTGAAGACTCAGCTAGAAAGACCCTTGCAAACATCCTGTGGCGAGAGCAAGGCCT
CAGCGTGGGAAACATGTTCTATGTCTTCTCCGACGACGGTATCATCGTCATCCATCCTGTGGACTGTGAG
ATCCAGAGGCACCTCAAACCCACGGAAAAGATTTTCATGAGCTATGAAGAAATCTGTCCTCAAAGAGAAA
AAAATGCAACCCAGCCCTGCCAGTGGGTATCTGCAGTCAATGTCCGGAACCGGTACATCTATGTGGCCCA
GCCAGCACTGAGCAGAGTCCTTGTGGTCGACATCCAAGCCCAGAAAGTCCTACAGTCCATAGGTGTGGAC
CCTCTGCCGGCTAAGCTGTCCTATCACAAGTCACATGACCAAGTGTGGGTCCTGAGCTGGGGGGACGTGC
ACAAGTCCCGACCAAGTCTCCAGGTGATCACAGAAGCCAGCACCGGCCAGAGCCAGCACCTCATCCGCAC
ACCCTTTGCAGGAGTGGATGATTTCTTCATTCCCCCAACAAACCTCATCATCAACCACATCAGGTTTGGC
TTCATCTTCAACAAGTCTGATCCTGCAGTCCACAAGGTGGACCTGGAAACAATGATGCCCCTCAAGACCA
TCGGCCTGCACCACCATGGCTGCGTGCCCCAGGCCATGGCACACACCCACCTGGGCGGCTACTTCTTCAT
CCAGTGCCGACAGGACAGCCCCGCCTCTGCTGCCCGACAGCTGCTCGTTGACAGTGTCACAGACTCTGTG
CTTGGCCCCAATGGTGATGTAACAGGCACCCCACACACATCCCCCGACGGGCGCTTCATAGTCAGTGCTG
CAGCTGACAGCCCCTCCCTGCACGTGCAGGAGATCACAGTGCGGGCCGAGATCCAGACCCTGTATGACCT
GCAAATAAACTCGGGCATCTCAGACTTGGCCTTCCAGCGCTCCTTCACTGAAAGCAATCAATACAACATC
TACGCGGCTCTGCACACGGAGCCGGACCTGCTGTTCCTGGAGCTGTCCACGGGGAAGGTGGGCATGCTGA
AGAACTTAAAGGAGCCACCCGCAGGGCCAGCTCAGCCCTGGGGGGGTACCCACAGAATCATGAGGGACAG
TGGGCTGTTTGGACAGTACCTCCTCACACCACCCCGAGAGTCACTGTTCCTCATCAATGCGAGACAAAAC
ACGCTGCGGTGTGAGGTGTCAGGTATAAAGGGGGGGACCACACTGGTGTGGGTGGGTGAGGTATGAAGGG
CCCAGAGCAGAGCCCTGGGCCAAGGAACACCCCCTAGTCCTGACACTGCAGCCTCAAGCAGGTACGCTGT
ACATTTTTACAGACAAAAGCAAAAACCTGTACTCGCTTTGTGGTTCAACACTGGTCTCCTTGCAAGTTTC
CTAGTATAAGGTATGCGCTGCTACCAAGATTGGGGTTTTTTCCTTAGGAAGTATGATTTATGCCTTGAGC
TACCATGAGAACATATGCTGCTGTGTAAAGGGATCATTTCTGTGCCAAGCTGCACACCGAGTGACCTCGC
GACATCATGGAACCAAGGGATCCTGCTCTCCAAGCAGACACCTCTGTCAGTTGCCTTCACATAGTCATTG
TCCCTTACTGCCAGACCCAGCCAGACTTTGCCCTGACGGAGTGGCCCCGAAGCAGAGGCCGACCAGGAGC
AGGGGCCTCCCTCCCGAACTGAAAGCCCATCCGTCCTCGCGTGGGACCGCATCTTCTCCCTCCCAGCTGC
TTCTTGCTTTTCTTTCCATTTGACTTGCTGTAAGCCTGAGGGAGAGCCAACAAGACTTACTGCATCTTGG
GGGATGGGGAAATCACTCACTTTATTTTGGAAATTTTTGATTAAAAAAAAAATTTTATAATCTCAATGCT
AGTAAGCAGAAAGATCCTCTCCGAGGTCCAACTATATCCTTCCCTGCCTTAGGCCCAGTCTCGGGGGTGG
TCACAACCCCACATCCCACAGCCAGAAAGAACAATGGTCATCTGAGAATACTGGCCCTGTCGACTATTGC
CACCCTGCTTCTCCAAGAGCAGACCAGGCCACCTCATCCGTAAGGACTCGGTTCTGTGTTGGGACCCCAA
AAAACCAGAACAAGTTCTGTGTGCCTCCTTTCAGCACAGAAGGGACACATCTCATTAGTCAGGTCTGCTA
CCCCAGATTCAGGGCAGACTGGGCTTGCCTGGCAAGGTATGGGTGGCCTCCAGGCTCAATGCAGAAACCC
CAAGGACACGAGTGGGGCCAGGTGAGTTCCTGAAGCTATACCTTTTCAAAACAGATTTTGTTTTCCTACC
TGTGGCCCATCCACTCCTCTCTGGTACCCCATCCCCGCATCAGCACTCCAGAGAGAACACATTTCGGCGA
GGGTTTTCTTACCCACATTCCCCAATCAATACACACACACTGCACAACCCAGAACAGAAGGCCACAGGCT
GGCACTACTGCATTCTCCTTATGTGTCTCAGGCTGTGGTGACTCTCACATGGGCATCGAAGAAGTACAAC
CCACATAGCCCTCTGGAGACCGCCTAGATCAGAGACTCAGCAAAAACAGGCTCGCCTTCCCTCTCCCACA
TATGAGTGGAACTTACATGTGTCCTGGTTTGAATGATCATTTTGCAAGCCACACGGGTTCGGAGAGGTGG
TCTCACCACAGACGTCTTTGCTAATTTGGCCACCTTCACCTACTGACATGACCAGCATTTTCCTTTGCCA
TTAAGGAATGAACTCTTTCAAGGAGAGGAAACCCTAGACTCTGTGTCACTCTCAACACACACAGCTCCTT
TCACTCCTGCCTGACTGCCAAGCCACCTGCATCCCCCGCCCCAGATCTCATGAGATCAATCACTTGTATG
TCTCACGCAACTTGGTCCACCAAACGCCTGTCCCCTGTAACTCCTAGGGGTGCGCCTAGACAGGTACGTC
TGTTTTTTATTTTAAAAGATATGCTATGTAGATATAAGTTGAGGAAGCTCACCTCAAAAGCCTAAAATGC
AGTTTCACAGTAGCTGGGATGCATGGATGACCCATCTCACCCCTTTTTTTTTCCTGCCTCAATATCTTGA
TATGTTATGTTTACTCCCAATCTCCCATTTTTACCACTAAAATTCTCCAACTTTCATAAACTTTTTTTTG
GAAAAATTTCCATTGTATCAGCCCCTGACAGAAAAAGGATCTCTGAGCCTAAAGGAGGAAAAGTCCCACC
AACTACCAGACCAGAACACGAGCCCCTCTGGGCAGCAGGATTCCTAAGTCAAAGACCAGTTTGACCCAAA
CTCGCCTTTTAAAATAATCAGGAGTGACAGAGTCAACTTCTGCAGCACCTGCTTCTCCCCCACTGTCCCT
TCCATCTTGGAATGTGTCTAAAAAAGCATAGCTGCCCTTTGCTGTCCTCAGAGTGCATTTCCTGGAGACG
GCAGGCTTAGGTCTCACTGACAGCATGCCAGACACAACTGAATCGAAGCAGGCCTGAAGCCTAGGTCAGG
GTTTCAGGAGTCCAGCCCCAGGAGGCAAAGTCACCAATGCAGGGAGGTAAATGCCTTTTGGCAGGAAAAC
CAATAGAGTTGGTTGGGTGGGGAGTCAGGGGTGGGAGGAGAAGGAGGAAGAGGAGGAAGGCCAGACTGGC
CTGCCCTTTCTCCCATACTTCACCCCAGCAGAGGTTCATGGGACACAGTTGGAAAGCCACTGGGAGGAAA
TGCCTCACTACAGGGGGGCCTCCTGTAGCAAGCCCAGCCGGTAATCCTCCTAATGAACCCACAAGGTCAA
TTCACAACTGATATCTTAGCTATTAAAGAAGTACTGACTTTACCAAAAAAGAATCATCAAGA
AAGCTATTTATATAAACCCCCTCAGTCATTTTGAAATAAAATTAATTTTACAA
NOV17r,
13382322 SNP
for CG52643-02 SNP: Gly to Glu
Protein Sequence SEQ ID NO: 274 842 aa at position 37
MKPGGFWLHLTLLGASLPAALGWMDPGTSRGPDVGVEESQAEEPRSFEVTRREGLSSHNELLASCGKKFC
SRGSRCVLSRKTGEPECQCLEACRPSYVPVCGSDGRFYENHCKLHRAACLLGKRITVIHSKDCFLKGDTC
TMAGYARLKNVLLALQTRLQPLQEGDSRQDPASQKRLLVESLFRDLDADGNGHLSSSELAQHVLKKQDLD
EDLLGCSPGDLLRFDDYNSDSSLTLREFYAAFQAAQLSLAPEDRVSVTTVTVGLSTAATCAAAGDLRPPI
IWKRNGLTLNFLDLEDINDFGEDDSLYITKVTTIHMGNYTCHASGHEQLFQTHVLQVNVPPVIRVYPESQ
AQEPGVAASLRCHAEGIPMPRITWLKNGVDVSTQMSKQLSLLANCSELHISSVRYEDTGAYTCIAKNEVG
VDEDISSLFIEDSARKTLANILWREEGLSVGNMFYVFSDDGIIVIHPVDCEIQRHLKPTEKIFMSYEEIC
PQREKNATQPCQWVSAVNVRNRYIYVAQPALSRVLVVDIQAQKVLQSIGVDPLPAKLSYDKSHDQVWVLS
WGDVHKSRPSLQVITEASTGQSQHLIRTPFAGVDDFFIPPTNLIINHIRFGFIFNKSDPAVHKVDLETMM
PLKTIGLHHHGCVPQAMAHTHLGGYFFIQCRQDSPASAARQLLVDSVTDSVLGPNGDVTGTPHTSPDGRF
IVSAAADSPWLHVQEITVRGEIQTLYDLQINSGISDLAFQRSFTESNQYNIYAALHTEPDLLFLELSTGK
VGMLKNLKEPPAGPAQPWGGTHRIMRDSGLFGQYLLTPARESLFLINGRQNTLRCEVSGIKGGTTVVWVG
EV
NOV17s,
13382324 SNP
for CG52643-02 SEQ ID NO: 275 5573 bp SNP: 880 G/A
DNA Sequence ORF Start: ATG at 408 ORF Stop: end of sequence
GGAGAGGGCTGCATTGCTGTTGCTCACTGACCTTCTTTTATGCTGGCCTTTGGTTCAGAATGGCACATCA
TTCCTCGTTTTTGGCCCTCCAGCTGAACACCTGTTCTCTGTGGCACTGACTCCTCTTTCCATAGGGACAT
CATACAACAGTCGCCTTTATCTGAGGTTGTGCAAAGAGGGATGGAGGAGAAAACAATGGAGAATCCCTGG
CAGATTTCCCCAGGACGAGAGAAGGATATCCAATTGCTCATCAGGGAAGGTGCTAGGTCTCCCAGCCAGA
CGCCCTCAGAGGCCGGTGTCAAGTCTCCCTCACCTCTGTGATGTGAAGTCAGCTCGTTCATGACCTGGGC
AGGCAGAGGGTCAGAGGGGCAGATGGAGCACTCCTGGCCTGATGAAGACTCATCAAAATGAAACCAGGAG
GCTTTTGGCTGCATCTCACACTGCTCGGAGCCTCCCTGCCGGCTGCGCTGGGATGGATGGACCCAGGAAC
CAGCAGAGGCCCGGATGTGGGTGTGGGGGAGTCACAGCCAGAGGAGCCCAGAAGCTTTGAAGTCACAAGA
AGAGAAGGGCTTTCCAGCCACAACGACCTGCTGGCCTCCTGCCGGAAGAAGTTCTGCAGCCGACGGAGCC
GGTGCGTGCTCAGCAGGAAGACACGGGAGCCCGAATGCCAGTGCCTGGACGCATGCAGGCCCAGCTACGT
GCCTGTGTGCGGCTCTGATGGGAGGTTTTATGAAAACCACTGTAAGCTCCACCGTGCTGCTTGCCTCCTG
GGAAAGAGGATCACCGTCATCCACAGCAAGGACTGTTTCCTCAAACGTGACACGTGCACCATGGCCGGCT
ACGCCCGCTTGAAGAATGTCCTTCTGGCACTCCAGACCCATCTGCAGCCACTCCAAGAAGGAGACAGCAG
ACAAGACCCTGCCTCCCAGAAGCGCCTCCTGGTGGAATCTCTGTTCAGGGACTTAGATGCAGATGGCAAT
GGCCACCTCAGCAGCTCCGAACTGGCTCAGCATGTGCTGAACAAGCAGGACCTGCATGAAGACTTACTTG
GTTGCTCACCAGGTGACCTCCTCCGATTTGACGATTACAACACTGACAGCTCCCTGACCCTCCGCGAGTT
CTACATGGCCTTCCAAGTGGTTCAGCTCAGCCTCGCCCCCGAGGACAGGGTCAGTGTGACCACAGTGACC
GTGGGGCTGAGCACACTGCTGACCTGCCCCGTCCATGGAGACCTGAGGCCACCAATCATCTGGAAGCGCA
ACCGGCTCACCCTGAACTTCCTGGACTTGGAAGACATCAATGACTTTGGAGAGGATGATTCCCTGTACAT
CACCAAGGTGACCACCATCCACATGGGCAATTACACCTGCCATGCTTCCGGCCACGAGCAGCTGTTCCAG
ACCCACGTCCTGCAGGTGAATGTGCCGCCAGTCATCCGTGTCTATCCAGAGAGCCAGGCACAGGAGCCTG
GAGTGGCAGCCAGCCTAAGATGCCATGCTGAGGGCATTCCCATGCCCAGAATCACTTGGCTGAAAAACGG
CGTGGATGTCTCAACTCAGATGTCCAAACAGCTCTCCCTTTTAGCCAATGGGAGCCAACTCCACATCAGC
AGTGTTCGGTATGAAGACACAGGGGCATACACCTGCATTGCCAAAAATGAAGTGGGTGTGGATGAAGATA
TCTCCTCGCTCTTCATTGAAGACTCAGCTAGAAAGACCCTTGCAAACATCCTGTGGCGAGAGGAAGGCCT
CAGCGTGGGAAACATGTTCTATGTCTTCTCCGACGACGGTATCATCGTCATCCATCCTGTGGACTGTGAG
ATCCAGAGGCACCTCAAACCCACGGAAAAGATTTTCATGAGCTATGAAGAAATCTGTCCTCAAAGAGAAA
AAAATGCAACCCAGCCCTGCCAGTGGGTATCTGCAGTCAATGTCCGGAACCGGTACATCTATGTGGCCCA
GCCAGCACTGAGCAGAGTCCTTGTGGTCGACATCCAAGCCCAGAAAGTCCTACAGTCCATAGGTGTGGAC
CCTCTGCCGGCTAAGCTGTCCTATGACAAGTCACATGACCAAGTGTGGGTCCTGAGCTGGGGCGACGTGC
ACAAGTCCCGACCAAGTCTCCAGGTGATCACAGAAGCCAGCACCGGCCAGAGCCAGCACCTCATCCGCAC
ACCCTTTGCAGGAGTGGATGATTTCTTCATTCCCCCAACAAACCTCATCATCAACCACATCAGGTTTGGC
TTCATCTTCAACAAGTCTGATCCTGCAGTCCACAAGGTGGACCTGGAAACAATGATGCCCCTCAAGACCA
TCGGCCTGCACCACCATGGCTGCGTGCCCCAGGCCATGGCACACACCCACCTGGGCGGCTACTTCTTCAT
CCAGTGCCGACAGGACAGCCCCGCCTCTGCTGCCCGACAGCTCCTCGTTGACAGTGTCACAGACTCTGTG
CTTGGCCCCAATGGTGATGTAACAGGCACCCCACACACATCCCCCGACGGGCGCTTCATAGTCAGTGCTG
CAGCTGACAGCCCCTGGCTGCACGTGCAGCAGATCACAGTGCGGGGCGAGATCCAGACCCTGTATGACCT
GCAAATAAACTCGGGCATCTCAGACTTGGCCTTCCAGCGCTCCTTCACTGAAAGCAATCAATACAACATC
TACGCGGCTCTGCACACGGAGCCGGACCTGCTGTTCCTGGAGCTGTCCACGGGGAAGGTGGGCATGCTGA
AGAACTTAAAGGAGCCACCCGCAGGGCCAGCTCAGCCCTGGGGGGGTACCCACAGAATCATGAGGGACAG
TGGGCTGTTTGGACAGTACCTCCTCACACCAGCCCGAGAGTCACTGTTCCTCATCAATGGGAGACAAAAC
ACGCTGCGCTGTGAGGTGTCAGGTATAAAGGGGGCGACCACAGTGGTGTGGGTGGGTGAGGTATGAAGGG
CCCAGAGCAGAGCCCTGGGCCAAGGAACACCCCCTAGTCCTGACACTGCAGCCTCAAGCAGGTACGCTGT
ACATTTTTACAGACAAAAGCAAAAACCTGT
ACTCGCTTTGTGGTTCAACACTGGTCTCCTTGCAAGTTTCCTAGTATAAGGTATGCGCTGCTACCAAGAT
TGGGGTTTTTTCGTTAGGAAGTATGATTTATGCCTTGAGCTACGATGAGAACATATGCTGCTGTGTAAAG
GCATCATTTCTGTGCCAAGCTGCACACCGAGTGACCTGGGGACATCATGGAACCAAGGGATCCTGCTCTC
CAAGCAGACACCTCTGTCAGTTGCCTTCACATAGTCATTGTCCCTTACTGCCAGACCCAGCCAGACTTTG
CCCTGACGGAGTGGCCCGGAGCAGAGAACCGACCAGGAGCAGGGGCCTCCCTCCCGAACTGAAAGCCCAT
CCGTCCTCGCGTGGGACCGCATCTTCTCCCTCGCAGCTGCTTCTTGCTTTTCTTTCCATTTGACTTGCTG
TAAGCCTGAGCGAGAGCCAACAAGACTTACTGCATCTTGGGGGATGGGGAAATCACTCACTTTATTTTGG
AAATTTTTGATTAAAAAAATTTTATAATCTCAAATGCTAGTAAGCAGAAAAAGATGCTCTCCGAGGTCCA
ACTATATCCTTCCCTGCCTTAGGCCGAGTCTCGGGGGTGGTCACAACCCCACATCCCACAGCCAGAAAGA
ACAATGGTCATCTGAGAATACTGGCCCTGTCGACTATTGCCACCCTGCTTCTCCAAGAGCAGACCAGGCC
ACCTCATCCGTAAGGACTCGGTTCTGTGTTGGGACCCCAAAAAACCAGAACAAGTTCTGTGTGCCTCCTT
TCAGCACAGAAGGGAGACATCTCATTAGTCAGGTCTGGTACCCCAGATTCAGGGCAGACTGGGCTTGCCT
GGCAAGGTATGGGTGGCCTCCAGGCTCAATGCAGAAACCCCAAGGACACGAGTGGGGCCAGGTGAGTTCC
TGAAGCTATACCTTTTCAAAACAGATTTTGTTTTCCTACCTGTGGCCCATCCACTCCTCTCTGGTACCCC
ATCCCCGCATCAGCACTGCAGAGAGAACACATTTCGGCGAGGGTTTTCTTACCCACATTCCCCAATCAAT
ACACACACACTGCAGAACCCAGAACAGAAGGCCACAGGCTGGCACTACTGCATTCTCCTTATGTGTCTCA
GGCTGTGGTGACTCTCACATGGGCATCGAAGAAGTACAACCCACATAGCCCTCTGGAGACCGCCTAGATC
ACAGACTCAGCAAAAACAGGCTCGCCTTCCCTCTCCCACATATGAGTGGAACTTACATGTGTCCTGGTTT
GAATGATCATTTTGCAAGCCACACGGGTTGGGAGAGGTGGTCTCACCACAGACGTCTTTGCTAATTTGGC
CACCTTCACCTACTGACATGACCAGGATTTTCCTTTGCCATTAAGGAATGAACTCTTTCAAGGAGAGGAA
ACCCTAGACTCTGTGTCACTCTCAACACACACAGCTCCTTTCACTCCTGCCTGACTGCCAAGCCACCTGC
ATCCCCCGCCCCAGATCTCATGAGATCAATCACTTGTATGTCTCACGCAACTTGGTCCACCAAACGCCTG
TCCCCTGTAACTCCTAGGGGTGCGCCTAGACAGGTACGTCTGTTTTTTATTTTAAAAGATATGCTATGTA
GATATAAGTTGAGGAAGCTCACCTCAAAAGCCTAGAATGCAGTTTCACAGTAGCTGGGATGCATGGATGA
CCCATCTCACCCCTTTTTTTTTCCTGCCTCAATATCTTGATATGTTATGTTTACTCCCAATCTCCCATTT
TTACCACTAAAATTCTCCAACTTTCATAAACTTTTTTTTGGAAAAATTTCCATTGTATCAGCCCCTGACA
GAAAAAGGATCTCTGAGCCTAAAGGAGGAAAAGTCCCACCAACTACCAGACCAGAACACGAGCCCCTCTG
GGCAGCAGGATTCCTAAGTCAAAGACCAGTTTGACCCAAACTGGCCTTTTAAAATAATCAGGAGTGACAG
AGTCAACTTCTGCAGCACCTGCTTCTCCCCCACTGTCCCTTCCATCTTGGAATGTGTCTAAAAAAGCATA
GCTCCCCTTTGCTGTCCTCAGAGTGCATTTCCTCGAGACGGCAGGCTTAGGTCTCACTGACAGCATGCCA
GACACAACTGAATCGAAGCAGGCCTGAAGCCTAGGTCAGGGTTTCAGGAGTCCAGCCCCAGGAGGCAAAG
TCACCAATGCAGGGAGGTAAATGCCTTTTGGCAGGAAAACCAATAGAGTTGCTTGGGTGGGGAGTCAGGG
GTGGGAGGAGAAGGAGGAAGAGGAGGAAGGCCAGACTGGCCTGCCCTTTCTCCCATACTTCACCCCAGCA
CAGGTTCATGGGACACAGTTGGAAAAGCCACTGGGAGGAATGCCTCACTACAGGGGGGCCTCCTGTAGCA
AGCCCAGCCGGTAATCCTCCTAATGAACCCACAAGGTCAATTCACAACTGATATCTTAGCTATTAAACAA
GTACTGACTTTACCAAAAGAATCATCAAGAAAGCTATTTATATAAACCCCCTCAGTCATTTTGAAATAAA
ATTAATTTTACAA
NOV17s,
13382324 SNP
for CG52643-02 SNP: Arg to His
Protein Sequence SEQ ID NO: 276 842 aa at position 158
MKPGGFWLHLTLLGASLPAALGWMDPGTSRGPDVGVGESQAEEPRSFEAARREGLSSHNELLASCGKKFC
SRGSRCVLSRKTGEPECQCLEACRPSYVPVCGSDGRFYENHCKLHRAACLLGKRITVIHSKDCFLKGDTC
TMAGYARLKNVLLALQTHLQPLQEGDSRQDPASQAALLVESLFRDLDADGNGNLSSSELAQHVLKKQDLD
EDLLGCSPGDLLRFDDYNSDSSLTLREFYMAFQVVQLSLAPEDRVSVTTVTVGLSTVLTCAVHGDLRPPI
IWKRNGLTLNFLDLEDINDFGEDDSLYITKAATIHMGNYTCHASGHEQLFQTHAAQAAVPPVIRVYPESQ
AQEPGVAASLRCHAEGIPMPRITWLKNGVDVSTQMSKQLSLLANGSELHISSVRYEDTGAYTCIAKNEVG
VDEDISSLFIEDSARKTLANILWREECLSVGNMFYVFSDDGIIVIHPVDCEIQRHLKPTEKIFMSYEEIC
PQREKNATQPCQWVSAVNVRNRYIYVAQPALSRVLAADIQAQKVLQSIGVDPLPAKLSYDKSHDQVWVLS
WGDVHKSRPSLQVITEASTGQSQHLIRTPFAGAADFFIPPTNLIINHIRFGFIFNKSDPAVHKVDLETMM
PLKTIGLHHHGCVPQAMAHTHLGGYFFIQCRQDSPASAARQLLVDSVTDSVLGPNGDAAGTPHTSPDGRF
IVSAAADSPWLHVQEITVRGEIQTLYDLQINSGISDLAFQRSFTESNQYNIYAALHTEPDLLFLELSTGK
VGMLKNLKEPPAGPAQPWGGTHRIMRDSGLFGQYLLTPARESLFLINGRQNTLRCEVSGIKGGTTVVWVG
EV
NOV17t,
13381678 SNP
for CG52643-02 SEQ ID NO: 277 5573 bp SNP: 2677 C/T
DNA Sequence ORF Start: ATG at 408 ORF Stop: end of sequence
CGAGAGGGCTCCATTGCTGTTGCTCACTGACCTTCTTTTATCCTGGCCTTTGGTTCAGAATGGCACATCA
TTCCTCGTTTTTGGCCCTCCAGCTGAACACCTGTTCTCTGTGGCACTGACTCCTCTTTCCATAGGGACAT
CATACAACAGTCGCCTTTATCTGAGGTTGTGCAAAAAGAGGGATGGAGGAGAACAATGGAGAATCCCTGG
CAGATTTCCCCAGGACGAGAGAAGGATATCCAATTGCTCATCAGGGAAGGTGCTAGGTCTCCCAGCCAGA
CGCCCTCAGAGGCCGGTGTCAACTCTCCCTCACCTCTGTCATGTGAAGTCAGCTCGTTCATGACCTGGGC
AGGCAGAGGGTCAGAGGGGCAGATGGAGCACTCCTGGCCTGATGAAGACTCATCAAAATGAAACCAGGAG
GCTTTTGGCTGCATCTCACACTGCTCGGAGCCTCCCTGCCGGCTGCGCTGGGATGGATGG
ACCCAGUAACCAGCAGAGGCCCGGATGTGGGTGTGGGGGAGTCACAGGCAGACGAGCCCAGAAGCTTTGA
AGTCACAAGA
AGAGAAGGGCTTTCCAGCCACAACGAGCTGCTGGCCTCCTGCGGGAAGAAGTTCTGCAGCCGAGGGAGCC
GGTGCGTGCTCAGCAGGAAGACAGGGGAGCCCGAATCCCAGTGCCTGGAGGCATGCAGGCCCAGCTACGT
GCCTGTGTGCGGCTCTGATGGGAGGTTTTATGAAAACCACTGTAAGCTCCACCGTGCTGCTTGCCTCCTG
GGAAAGAGGATCACCGTCATCCACAGCAAGGACTGTTTCCTCAAAGGTGACACGTGCACCATGGCCGGCT
ACGCCCGCTTGAAGAATGTCCTTCTGGCACTCCAGACCCGTCTGCAGCCACTCCAAGAAGGAGACACCAG
ACAAGACCCTGCCTCCCAGAAGCGCCTCCTGGTGGAATCTCTGTTCAGGGACTTAGATGCAGATCGCAAT
GCCCACCTCAGCAGCTCCGAACTCGCTCAGCATGTGCTGAAGAAGCAGGACCTGGATGAAGACTTACTTG
GTTGCTCACCAGGTGACCTCCTCCGATTTGACGATTACAACAGTGACAGCTCCCTGACCCTCCGCGAGTT
CTACATGGCCTTCCAAGTGGTTCAGCTCACCCTCGCCCCCGAGGACAGGGTCAGTGTGACCACAGTGACC
GTGGGGCTGAGCACAGTGCTGACCTGCGCCGTCCATGGAGACCTGAGGCCACCAATCATCTGGAAGCGCA
ACGGGCTCACCCTGAACTTCCTGGACTTGGAAGACATCAATGACTTTGGAGAGGATGATTCCCTGTACAT
CACCAAGGTGACCACCATCCACATGGGCAATTACACCTGCCATGCTTCCGGCCACGAGCAGCTGTTCCAG
ACCCACGTCCTGCAGGTGAATGTGCCGCCAGTCATCCGTGTCTATCCAGAGAGCCAGGCACAGGAGCCTG
GAGTGGCAGCCAGCCTAAGATGCCATGCTGAGGGCATTCCCATGCCCAGAATCACTTGGCTGAAAAACGG
CGTGGATGTCTCAACTCAGATGTCCAAACAGCTCTCCCTTTTAGCCAATGGGAGCGAACTCCACATCAGC
AGTGTTCCGTATGAAGACACAGGGGCATACACCTGCATTGCCAAAAATGAAGTGGGTGTGGATGAAGATA
TCTCCTCGCTCTTCATTGAAGACTCAGCTAGAAAGACCCTTGCAAACATCCTGTCGCGAGAGGAAGGCCT
CAGCGTGGGAAACATGTTCTATGTCTTCTCCGACGACGGTATCATCGTCATCCATCCTGTGGACTGTGAG
ATCCAGAGGCACCTCAAACCCACGGAAAAGATTTTCATGAGCTATGAAGAAATCTGTCCTCAAAGAGAAA
AAAATGCAACCCAGCCCTGCCAGTGGGTATCTGCAGTCAATGTCCGGAACCGGTACATCTATGTGGCCCA
GCCAGCACTGAGCAGAGTCCTTGTGGTCCACATCCAAGCCCAGAAAGTCCTACAGTCCATAGGTGTGGAC
CCTCTGCCCGCTAAGCTGTCCTATGACAAGTCACATGACCAACTGTGGGTCCTGAGCTGGGGGGACGTGC
ACAAGTCCCGACCAAGTCTCCAGGTGATCACAGAAGCCAGCACCGGCCAGAGCCAGCACCTCATCCGCAC
ACCCTTTGCAGGAGTGGATGATTTCTTCATTCCCCCAACAAACCTCATCATCAACCACATCAGGTTTGGC
TTCATCTTCAACAAGTCTGATCCTGCACTCCACAAGGTGGACCTGGAAACAATGATGCCCCTCAAGACCA
TCGGCCTGCACCACCATGGCTGCGTGCCCCAGGCCATGGCACACACCCACCTGGGCGGCTACTTCTTCAT
CCAGTGCCGACAGCACAGCCCCGCCTCTGCTGCCCGACAGCTGCTCGTTGACAGTGTCACAGACTCTGTG
CTTGGCCCCAATGGTGATGTAACAGGCACCCCACACACATCCCCCGACGGGCGCTTCATAGTCAGTGCTG
CAGCTGACAGCCCCTGGCTGCACGTCCAGGAGATCACAGTGCGGGGCGAGATCCAGACCCTGTATGACCT
GCAAATAAACTCGGGCATCTCAGACTTGGCCTTCCAGCGCTCCTTCACTGAAAGCAATCAATACAACATC
TACGCGGCTCTGCACATGGAGCCCGACCTGCTGTTCCTGGAGCTGTCCACGGGGAAGGTGGGCATGCTGA
AGAACTTAAAGGAGCCACCCGCAGGGCCAGCTCAGCCCTGGGGGGGTACCCACAGAATCATGAGGGACAG
TGCGCTGTTTGGACAGTACCTCCTCACACCAGCCCGAGAGTCACTGTTCCTCATCAATGGGAGACAAAAC
ACGCTGCGGTGTGAGGTGTCAGGTATAAAGGGGGGGACCACAGTGGTGTGGGTGGGTGAGGTATGAAGGG
CCCAGAGCAGAGCCCTGGGCCAAGGAACACCCCCTAGTCCTGACACTGCAGCCTCAAGCAGGTACGCTGT
ACATTTTTACAGACAAAAGCAAAACCTGTACTCGCTTTGTGGTTCAACACTGGTCTCCTTGCAAAGTTTC
CTAGTATAAGGTATGCGCTGCTACCAAGATTGGGGTTTTTTCGTTAGGAAGTATGATTTATGCCTTGAGC
TACGATGAGAACATATGCTGCTGTGTAAAGGGATCATTTCTGTGCCAAGCTGCACACCGAGTGACCTGGG
CACATCATGGAACCAAGGGATCCTGCTCTCCAAGCAGACACCTCTGTCAGTTGCCTTCACATAGTCATTG
TCCCTTACTGCCAGACCCAGCCAGACTTTGCCCTGACGGAGTGGCCCGGAAGCAGAGGCCGACCAGGAGC
AGGGGCCTCCCTCCCGAACTGAAAGGCCATCCGTCCTCGCGTGGGACCGCATCTTCTCCCTCGCAGCTGC
TTCTTGCTTTTCTTTCCATTTGACTTGCTGTAAGCCTGAGGGAGAGCCAACAAGACTTACTGCATCTTGG
GGGATGGGGAAATCACTCACTTTATTTTGGAAATTTTTGATTAAAAAAATTTTATAATCTCAAAAATGCT
AGTAAGCAGAAAGATGCTCTCCGAGGTCCAACTATATCCTTCCCTGCCTTAGGCCCAGTCTCGGGGGTGG
TCACAACCCCACATCCCACAGCCAGAAAGAACAATGGTCATCTGAGAATACTGGCCCTGTCGACTATTGC
CACCCTGCTTCTCCAAGAGCAGACCAGGCCACCTCATCCGTAAGGACTCGGTTCTGTGTTGGGACCCCAA
AAAACCAGAACAAGTTCTGTGTGCCTCCTTTCAGCACAGAAGGGAGACATCTCATTAGTCAGGTCTGGTA
CCCCAGATTCAGGGCAGACTGGGCTTGCCTGGCAAGGTATGGGTGGCCTCCAGGCTCAATGCAGAAACCC
CAAGGACACGAGTGGGGCCAGGTGAGTTCCTGAAGCTATACCTTTTCAAAACAGATTTTGTTTTCCTACC
TGTGGCCCATCCACTCCTCTCTGGTACCCCATCCCCGCATCAGCACTGCAGAGAGAACACATTTCGGCGA
GGGTTTTCTTACCCACATTCCCCAATCAATACACACACACTGCAGAACCCAGAACAGAAGGCCACAGGCT
GGCACTACTGCATTCTCCTTATGTGTCTCAGGCTGTGGTGACTCTCACATGGGCATCGAAGAAGTACAAC
CCACATAGCCCTCTGGAGACCGCCTAGATCAGAGACTCAGCAAAAACAGGCTCGCCTTCCCTCTCCCACA
TATGAGTGGAACTTACATGTGTCCTGGTTTGAATGATCATTTTGCAAGCCACACGGGTTGGGAGAGGTGG
TCTCACCACAGACGTCTTTGCTAATTTGGCCACCTTCACCTACTGACATGACCAGGATTTTCCTTTGCCA
TTAAGGAATGAACTCTTTCAAGGAGAGGAAACCCTAGACTCTGTGTCACTCTCAACACACACAGCTCCTT
TCACTCCTGCCTGACTGCCAAUCCACCTGCATCCCCCGCCCCAGATCTCATGAGATCAATCACTTGTATG
TCTCACGCAACTTGGTCCACCAAACGCCTGTCCCCTGTAACTCCTAGGGGTGCGCCTAGACAGGTACGTC
TGTTTTTTATTTTAAAAGATATGCTATGTAGATATAAGTTGAGGAAGCTCACCTCAAAAGCCTAGAATGC
AGTTTCACAGTAGCTGGGATGCATGGATGACCCATCTCACCCCTTTTTTTTTCCTGCCTCAATATCTTGA
TATGTTATGTTTACTCCCAATCTCCCATTTTTACCACTAAAATTCTCCAACTTTCATAAACTTTTTTTTG
GAAAAATTTCCATTGTATCAGCCCCTGACACAAAAAGGATCTCTGAGCCTAAAGGAGGAAAAGTCCCACC
AACTACCAGACCAGAACACGAGCCCCTCTGGGCAGCAGGATTCCTAAGTCAAAGACCAGTTTGACCCAAA
CTGGCCTTTTAAAATAATCAGGAGTGACAGAGTCAACTTCTGCAGCACCTGCTTCTCCCCCACTGTCCCT
TCCATCTTGGAATGTGTCTAAAAAAGCATAGCTGCCCTTTGCTGTCCTCAGAGTGCATTTCCTGGAGACG
GCAGGCTTAGGTCTCACTGACAGCATGCCAGACACAACTGAATCGAAGCAGGCCTGAAGCCTAGGTCAGG
GTTTCAGGAGTCCAGCCCCAGGAGGCAAAGTCACCAATGCAGGGAGGTAAATGCCTTTTGGCAGGAAAAC
CAATAGAGTTGGTTGGGTGGGGAGTCAGGGGTGGGAGGAGAAGGAGGAAGAGGAGGAAGGCCAGACTGGC
CTGCCCTTTCTCCCATACTTCACCCCAGCAGAGGTTCATGGGACACAGTTGGAAAGCCACTGGGAGGAAA
TGCCTCACTACAGGGGGGCCTCCTGTAGCAAGCCCAGCCGGTATCCTCCTAATGAACCCACAAGGTCAA
TTCACAACTGATATAATTAGCTATTAAAGAAGTACTGACTTTACCAACAATCATCAAGAAAGCTATTTA
TATAAACCCCCTCAGTCATTTTGAAATAAAATTAATTTTACAA
NOV17t,
13381678 SNP
for CG52643-02 SNP: Thr to Met
Protein Sequence SEQ ID NO: 278 842 aa at position 757
MKPGGFWLHLTLLGASLPAALGWMDPGTSRGPDVGVGESQAEEPRSFEVTRREGLSSHNELLASCGKKF
CSRGSRCVLSRKTGEPECQCLEACRPSYVPVCGSDGRFYENHCKLHRAACLLGKRITVIHSKDCFLKCDT
CTMAGYARLKNVLLALQTRLQPLQEGDSRQDPASQKRLLVESLFRDLDADGNGHLSSSELAQHVLKKQDL
DEDLLGCSPGDLLRFDDYNSDSSLTLREFYMAFQVVQLSLAPEDRVSVTTVTVGLSTVLTCAVHGDLRPP
IIWKRNGLTLNFLDLEDINDFGEDDSLYITKVTTIHMGNYTCHASGHEQLFQTHVLQVNVPPVIRVYPES
QAQEPGVAASLRCHAEGIPMPRITWLKNGVDVSTQMSKQLSLLANGSELHISSVRYEDTCAYTCIAKNEV
CVDEDILSSLFIEDSARKTLANILWREEGLSVGNMFYVFSDDGHVIHPVDCEIQRHLKPTEKIFMSYEEI
CPQREKNATQPCQWVSAVNVRNRYIYVAQPALSRVLVVDIQAQKVLQSIGVDPLPAKLSYDKSHDQVWVL
SWGDVHKSRPSLQVITEASTGQSQHLIRTPFAGVDDFFTPPTNLIINHIRFGFIFNKSDPAVHKVDLETM
MPLKTIGLHHHGCVPQAMAHTHLGGYFFIQCRQDSPASAARQLLVDSVTDSVLGPNGDVTGTPHTSPDGR
FIVSAAADSPWLHVQEITVRGELQTLYDLQINSGISDLAFQRSFTESNQYNIYAALHMEPDLLFLELSTG
KVGMLKWLKEPPAGPAQPWGGTHRIMRDSGLFGQYLLTPARESLFLINGRQNTLRCEVSGIKGGTTVVWV
GEV

A ClustalW comparison of the above protein sequences yields the following sequence alignment shown in Table 17B.

TABLE 17B
Comparison of the NOV17 protein sequences.

| | |
|---|---|
| NOV17a | ------------------------------------------------------------ |
| NOV17b | ------------------------------------------------------------ |
| NOV17c | ------------------------------------------------------------ |
| NOV17d LEMKPGGFWLHLTLLGASLPAALGWMDPGTSRGPDVGVGESQAEEPRSFEVTRREGLSSH | |
| NOV17e | ------------------------------------------------------------ |
| NOV17f | ------------------------------------------------------------ |
| NOV17g | ------------------------------------------------------------ |
| NOV17h | ------------------------------------------------------------ |
| NOV17i | ------------------------------------------------------------ |
| NOV17j | ------------------------------------------------------------ |
| NOV17k | ------------------------------------------------------------ |
| NOV17l | ------------------------------------------------------------ |
| NOV17m | ------------------------------------------------------------ |
| NOV17n | ------------------------------------------------------------ |
| NOV17o | ------------------------------------------------------------ |
| NOV17p | ------------------------------------------------------------ |
| NOV17q | ------------------------------------------------------------ |
| NOV17a | ------------------------------------------------------------ |
| NOV17b | ------------------------------------------------------------ |
| NOV17c | ------------------------------------------------------------ |
| NOV17d | NELLASCGKKFCSRGSRCVLSRKTGEPECLGKRITVIHSKDCFLKGDTCTMAGYARLKNV |
| NOV17e | ------------------------------------------------------------ |
| NOV17f | ------------------------------------------------------------ |
| NOV17g | ------------------------------------------------------------ |
| NOV17h | ------------------------------------------------------------ |
| NOV17i | ------------------------------------------------------------ |
| NOV17j | ------------------------------------------------------------ |
| NOV17k | ------------------------------------------------------------ |
| NOV17l | ------------------------------------------------------------ |
| NOV17m | ------------------------------------------------------------ |
| NOV17n | ------------------------------------------------------------ |
| NOV17o | ------------------------------------------------------------ |
| NOV17p | ------------------------------------------------------------ |
| NOV17q | ------------------------------------------------------------ |
| NOV17a | ------------------------------------------------------------ |
| NOV17b | ------------------------------------------------------------ |
| NOV17c | ------------------------------------------------------------ |
| NOV17d | LLALQTRLQPLQEGDSRQDPASQKRLLVESLFRDLDADGNGHLSSSELAQHVLKKQDLDE |
| NOV17e | ------------------------------------------------------------ |
| NOV17f | ------------------------------------------------------------ |
| NOV17g | ------------------------------------------------------------ |
| NOV17h | ------------------------------------------------------------ |
| NOV17i | ------------------------------------------------------------ |
| NOV17j | ------------------------------------------------------------ |
| NOV17k | ------------------------------------------------------------ |
| NOV17l | ------------------------------------------------------------ |
| NOV17m | ------------------------------------------------------------ |
| NOV17n | ------------------------------------------------------------ |
| NOV17o | ------------------------------------------------------------ |
| NOV17p | ------------------------------------------------------------ |
| NOV17q | ------------------------------------------------------------ |
| NOV17a | ------------------------------------------------MKPGGFWLHLTL |
| NOV17b | ----------------------------------------------LEMKPGGFWLHLTL |
| NOV17c | ----------------------------------------------LEMKPGGFWLHLTL |
| NOV17d | DLLGCSPGDLLRFDDYNSDSSLTLREFYMAFQVVQLSLAPEDRVSVTTVGLSTVLTCA |
| NOV17e | ------------------------------------------------------------ |
| NOV17f | ----------------------------------------------LEMKPGGFWLHLTL |
| NOV17g | ------------------------------------------------MKPGGFWLHLTL |
| NOV17h | --------------------------------------------TGSTMKPGGFWLHLTL |
| NOV17i | -------------------------------------------------LEGLSTVLTCA |
| NOV17j | -------------------------------------------------LEGLSTVLTCA |
| NOV17k | -------------------------------------------------LEGLSTVLTCA |
| NOV17l | ------------------------------------------------------------ |
| NOV17m | ------------------------------------------------------------ |
| NOV17n | ------------------------------------------------------------ |
| NOV17o | ------------------------------------------------------------ |
| NOV17p | ------------------------------------------------MKPGGFWLHLTL |
| NOV17q | ------------------------------------------------------------ |
| NOV17a | LGASLPAALGWMDPGTSRGPDVGVGESQAEEPRSFEVTRREGLSSHNELLASCGKKFCSR |
| NOV17b | LGASLPAALGWMDPGTSRGPDVGVGESQAEEPRSFEVTRREGLSSHNELLASCGKKFCSR |
| NOV17c | LGASLPAALCWMDPGTSRGPDVGVCESQAEEPRSFEVTRREGLSSHNELLASCGKKFCSR |
| NOV17d | VHGDLRPPIIWKRNGLTLNFLDLEDINDFGEDDSLYITKVTTIHMCNYTCHASGHEQLFQ |
| NOV17e | ------LEWMDPGTSRGPDVGVGESQAEEPRSFEVTRREGLSSHNELLASCGKKFCSR |
| NOV17f | LGASLPAALGWMDPGTSRGPDVGVGESQAEEFRSFEVTRREGLSSHNELLASCGKKFCSR |
| NOV17g | LGASLPAALGWMDPGTSRGPDVOVGESQAEEPRSFEVTRREGLSSHNELLASCGKKFCSR |
| NOV17h | LGASLPAALGWMDPGTSRGPDVCVGESQAEEPRSFEVTRREGLSSHNELLASCGKKFCSR |
| NOV17i | VHGDLRFPIIWKRNGLTLNFLDLEDINDFGEDDSLYITKVTTIHMGNYTCHASGHEQLFQ |
| NOV17j | VHGDLRPPIIWKPNGLTLNFLDLEDINDFGEDDSLYITKVTTIHMGNYTCHASGHEQLFQ |
| NOV17k | VHGDLRPPIIWKRNGLTLNFLDLEDINDFGEDDSLYITKVTTIHMGNYTCHASGHEQLFQ |
| NOV17l | -YLRFAFTGSWMDPGTSRGPDVGVGESQAEEPRSFEVTRRECLSSHNELLASCGKKFCSR |
| NOV17m | -------TGSWMDPGTSRGPDVGVGESQAEEPRSFEVTRREGLSSHNELLASCGKKFCSR |
| NOV17n | ------------------------------------------------------------ |
| NOV17o | ----------WMDPGTSRGPDVGVEESQAEEPRSFEVTRREGLSSHNELLASCGKKFCSR |
| NOV17p | LGASLPAALGWMDPGTSRGPDVGVGESQAEEPRSFEVTRRECLSSHNELLASCGKKFCSR |
| NOV17q | ----------WMDPGTSRGPDVOVGESQAEEPRSFEVTRREGLSSHNELLASCCKKFCSR |
| NOV17a | ----------GSRCVLSRKTGEP--------ECQCLEACRPSYVPVCGSDGRFYENHCKL |
| NOV17b | ----------GSRCVLSRKTGEP--------ECQCLEACRPSYVPVCGSDGRFYENHCKL |
| NOV17c | ----------GSRCVLSRKTCEP--------ECQCLEACRPSYVPVCGSDGRFYENHCKL |
| NOV17d | THVLQVNVPPVIRVYPESQAQEPGVAASLRCHAEGIPMPRITWLKNGVDVSTQMSKQLSL |
| NOV17e | ----------GSRCVLSRKTGEP--------ECQCLEACRPSYVPVCGSDGRFYENHCKL |
| NOV17f | ----------GSRCVLSRKTGEP--------ECQCLEACRPSYVPVCGSDGRFYENHCKL |
| NOV17g | ----------GSRCVLSRKTCEP--------ECQCLEACRPSYVPVCGSDGRFYENHCKL |
| NOV17h | ----------GSRCVLSRKTOEP--------ECQCLEACRPSYVPVCGSDGRFYENHCKL |
| NOV17i | THVLQVNVPPVIRVYPESQAQEPGVAASLRCHAEGIPMPRITWLKNGVOVSTQMSKQLSL |
| NOV17j | THVLQVNVPPVIRVYPESQAQEPGVAASLRCHAEGIPMPRITWLKNGVDVSTQMSKQLSL |
| NOV17k | THVLQVNVPPVIRVYPESQAQEPGVAASLRCHAEGIPMPRITWLKNGVDVSTQMSKQLSL |
| NOV17l | ----------GSRCVLSRKTGEP--------ECQCLEACRPSYVPVCGSDGRFYENHCKL |
| NOV17m | ----------GSRCVLSRKTGEP--------ECQCLEACRPSYVPVCGSOGRFYENHCKL |
| NOV17n | ------------------------------------------------------------ |
| NOV17o | ----------GSRCVLSRKTGEP--------ECQCLEACRPSYVPVCGSDGRFYENHCKL |
| NOV17p | ----------GSRCVLSRKTGEP--------ECQCLEACRPSYVPVCGSDGRFYENHCKL |
| NOV17q | ----------GSRCVLSRKTGEP--------ECQCLEACRPSYVPVCGSDGRFYENHCKL |
| NOV17a | HRAACLLGKRITVIHSKDCFLKGDTCTMAGYARLKNVLLALQTRLQPLQ----EGDSRQ- |
| NOV17b | HRAACLLGKRITVIHSKDCFLKGDTCTMAGYARLKNVLLALQTRLQPLQ----EGDSRQ- |
| NOV17c | HRAACLLGKRITVIHSKDCFLKGDTCTIAGYARLKNVLLALQTRLQPLQ----EGDSRQ- |
| NOV17d | LANGSELHISSVRYEDTGAYTCIAKNEVGVDEDISSLFIEDSARKTLANILWREE----- |
| NOV17e | HRAACLLGKRITVIHSKDCFLKGDTCTMAGYARLKNVLLALQTRLQFLQ----EGDSRQ- |
| NOV17f | HRAACLLGKRITVIHSKDCFLKGDTCTMAGYARLKNVLLALQTRLQPLQ----EGDSRQ- |
| NOV17g | HRAACLLGKRITVIHSKDCFLKGDTCTMAGYARLKNVLLALQTRLQPLQ----EGDSRQ- |
| NOV17h | HRAACLLGKRITVIHSKDCFLKGDTCTMAGYARLKNVLLALQTRLQPLQ----EGDSRQ- |
| NOV17i | LANGSELHISSVRYEDTGAYTCIAKNEVGVDEDISSLFIEDSARKTLANILWREEDASTW |
| NOV17j | LANGSELHISSVRYEDTGAYTCIAKNEVGVDEDISSLFIEDSARKTR------------- |
| NOV17k | LANGSELHISSVRYEDTGAYTCIAKNEVGVDEDISSLFIEDSARKTLANILWREE----- |
| NOV17l | HRAACLLGKRITVIHSKDCFLKGDTCTMAGYARLKNVLLALQTRLQPLQ----EGDSRQ- |
| NOV17m | HRAACLLGKRITVINSKDCFLKGDTCTMAGYARLKNVLLALQTRLQPLQ----EGDSRQ- |
| NOV17n | ---------------------------MAGYARLKNVLLALQTRLQPLQ----EGDSRQ- |
| NOV17o | HRAACLLGKRIAVIHSKDCFLKGDTCTMGGYARLKNVLLALQTRLQFLQ----EGDSRQ- |
| NOV17p | HRAACLLGKRITVIHSKDCFLKGDTCTMAGYARLKNVLLALQTRLQPLQ----EGDSRQ- |
| NOV17q | HRAACLLGKRITVIHSKDCFLKGDTCTMAGYARLKNVLLALQTRPQPLQ----EGDSRQ- |
| NOV17a | ----------DPASQK---R----------LLVESLFRDLDAD--GNGHLSSSELAQHVL |
| NOV17b | ----------DPASQK---R----------LLVESLFROLDAD--GNGHLSSSELAQHVL |
| NOV17c | ----------DPASQK---R----------LLVESLFRDLDAD--GNGHLSSSELAQHVL |
| NOV17d | -----------------------------GLSVGNMFYVFSDOGIIVIHPVDCEIQRHLK |
| NOV17e | ----------DPASQK---R----------LLVESLFRDLDAD--GNGHLSSSELAQHVL |
| NOV17f | ----------DPASQK---R----------LLVESLFRDLDAD--GNCHLSSSELAQHVL |
| NOV17g | ----------DPASQK---R----------LLVESLFRDLDAD--GNGHLSSSELAQHVL |
| NOV17h | ----------DPASQK---R----------LLVESLFRDLDAD--GNGHLSSSELAQHVL |
| NOV17i | PVSCVFNAACDPAQGPTAWRACPFHLLLPGLSVGNMFYVFSDDGIIVIHPVDCEIQRHLK |
| NOV17j | ------------------------------LSVGNMFYVFSDDGIIVIHPVDCEIQRHLK |
| NOV17k | -----------------------------GLSVGNMFYVFSDDCTIVIHPVDCEIQRHLK |
| NOV17l | ----------DPASQK---R----------LLVESLFRDLDAD--GNGHLSSSELAQHVL |
| NOV17m | ----------DPASQK---R----------LLVESLFRDLDAD--GNGHLSSSELAQHVL |
| NOV17n | ----------DPASQK---R----------LLVESLFRDLDAD--GNGHLSSSELAQHVL |
| NOV17o | ----------DPASQK---R----------LLVESLFRDLDAD--GNGHLSSSELAQHVL |
| NOV17p | ----------DPASQK---R----------LLVESLFRDLDAD--GNGHLSSSELAQHVL |
| NOV17q | ----------DPASQK---R----------LLVESLFRDLDAD--GNGHLSSSELAQHVL |
| NOV17a | KKQDLDEDLLGCSP---GDLLRFDDYNSDSSLTLREFYMAFQVVQLSLAPEDRVSVTTVT |
| NOV17b | KKQDLDEDLLGCSP---GDLLRFDDYNSDSSLTLREFYMAFQVVQLSLAPEDRVSVTTVT |
| NOV17c | KKQDLDEDLLGCSP---GDLLRFDDYNSDSSLTLREFYMAFQVVQLSLAPEDRVSVTTVT |
| NOV17d | PTEKIFMSYEEICPQREKNATQPCQWVSAVNVRNRYTYVAQPALSRVLVVDIQAQKVLQS |
| NOV17e | KKQDLDEDLLGCSP---GDLLRFDDYNSESSLTLREFYMAFQVVQLSLAPEDRVSVTTVT |
| NOV17f | KKQDLDEDLLGCSP---GDLLRFDDYNSDSSLTLREFYMAFQVVQLSLAPEDRVSVTTVT |
| NOV17g | KKQDLDEDLLGCSP---GDLLRFDDYNSDSSLTLREFYMAFQVVQLSLAPEDRVSVTTVT |
| NOV17h | KKQDLDEDLLGCSP---GDLLRFDDYNSDSSLTLREFYMAFQVVQLSLAPEDRVSVTTVT |
| NOV17i | PTEKTFMSYEEICPQREKNATQPCQWVSAVNVRNRYIYVAQPALSRVLVVDIQAQKVLQS |
| NOV17j | PTEKIFMSYEEICPQREKNATQPCQWVSAVNVRNRYIYVAQPALSRVLVVDIQAQKVLQS |
| NOV17k | PTEKIFMSYEEICPQREKNATQPCQWVSAVNVRNRYIYVAQPALSRVLVVDIQAQKVLQS |
| NOV17l | KKQDLDEDLLGCSP---GDLLRFDDYNSDSSLTLREFYMAFQVVQLSLAPEDRVSVTTVT |
| NOV17m | KKQDLDEDLLGCSP---GDLLRFDDYNSDSSLTLREFYMAFQVVQLSLAPEDRASVTTCT |
| NOV17n | KKQDLDEDLLGCSP---GDLLRFDDYNSDSSLTLREFYMAFQVVQLSLAPEDRVSVTTCT |
| NOV17o | KKQDLDEDLLGCSP---GDLLRFDDYNSDSSLTLREFYMAFQVVQLSLAPEDRVSVTTCT |
| NOV17p | KKQDLDEDLLGCSP---GDLLRFDDYNSDSSLTLREFYMAFQVVQLSLAPEDRVSVTTCT |
| NOV17q | KRQDLDEDLLOCSP---GDLLRFDDYNSDSSLTLREFYMAFQVVQLSLAPEDRVSVTTCT |
| NOV17a | VGLSTVLTCAVHGDLRPPIIWKRNGLTLN-FLDLEDINDFGEDDSLYITKVTTIHMGNYT |
| NOV17b | VGLSTVLTCAVHGDLRPPIIWKRNGLTLN-FLDLEDINDFGEDDSLYITKVTTIHMGNYT |
| NOV17c | VGLSTVLTCAVHGDLRPPIIWKRNGLTLN-FLDLEDINDFGEDDSLYITKVTTIHMGNYT |
| NOV17d | IGVDPLPAKLSYDKSHDQVWVLSWODVHKSRPSLQVITEASTGQSQHLIRTPFAGVDDFF |
| NOV17e | VGLSTVLTCAVHGDLRPPIIWKRNGLTLN-FLDLEDINDFGEDDSLYITKVTTIHMGNYT |
| NOV17f | VGLSTVLTCAVHGDLRPPIIWKRNGLTLN-FLDLEDINDFGEDDSLYITKVTTIHMGNYT |
| NOV17g | VGLSTVLTCAVHGDLRPPIIWKRNGLTLN-FLDLEDINDFGEDDSLYITKVTTIHMGNYT |
| NOV17h | VGLSTVLTCAVHGDLRPPIIWKRNOLTLN-FLDLEDINDFGEDDSLYITKVTTIHMGNYT |
| NOV17i | IGVDPLPAKLSYGKSHDQVWVLSWGDVHKSRPSLQVITEASTGQSQHLIRTPFAGVDDFF |
| NOV17j | IGVDPLPAKLSYDKSHDQVWVLSWGDVHKSRPSLQVITEASTGQSQNLIRTPFAGVDDFF |
| NOV17k | IGVDPLPAKLSYDKSHDQVWVLSWGDVHKSRPSLQVITEASTGQSQNLIRTPFAGVDDFF |
| N0V17l | VGLSTVLTCAVHGDLRPPIIWKRNGLTLN-FLDLEDINDFGED----------------- |
| NOV17m | VGLSTVLTCAVHGDLRPPIIWKRNGLTLN-FLDLEDINDFOEDDSLYITKVTTIHMGNYT |
| NOV17n | VGLSTVLTCAVHGDLRPPIIWKRNGLTLN-FLDLEDINDFGEDDSLYITKVTTIHMGNYT |
| NOV17o | VCLSTVPTCAVHGDLRPPIIWKRNGLTLN-FLDLEDINGR-------------------- |
| NOV17p | VGLSTVLTCAVHGDLRPPIIWKRNGLTLN-FLDLEDINDFGEDDSLYTTKVTTIHMGNYT |
| NOV17q | VGLSTVLTCAVHGDLRPPIIWKRNGLTLN-FLDLEDINDFGEDDSLYITKVTTIHMGNYT |
| NOV17a | CHASGHEQLFQTHVLQVNVPPVIRVYPESQAQEPGVAASLRCHAEGIPMPRITWLKNGVD |
| NOV17b | CHASGHEQLFQTHVLQVNVPPVIRVYPESQAQEPGVAASLRCHAEGIPMPRITWLKNGVD |
| NOV17c | CHASGHEQLFQTHVLQVNVPPVIRVYPESQAQEPGVAASLRCHAEGIPMPRITWLKNGVD |
| NOV17d | IPPTNLIINHIRFGFIFNKSDPAVHKVDLETNMPLKTIGLHNHGCVPQAMAHTHLGGYFF |
| NOV17e | CHASGHEQLFQTHVLQVNVPPVIRVYPESQAQEPGVAASLRCHAEGIPMPRITWLKNGVD |
| NOV17f | CHASGHEQLFQTHVLQVNVPPVIRWPESQAQEPGVAASLRCHAEGIPMPRITWLKNGVD |
| NOV17g | CHASGHEQLFQTHVLQVNVPPVIRVYPESQAQEPGVAASLRCHAEGIPMPRITWLKNGVD |
| NOV17h | CHASGNEQLFQTHVLQVNVPPVIRWPESQAQEPGVAASLRCHAEGIPMPRITWLKNGVD |
| NOV17i | IPPTNLIINHIRFGFIFNKSDPAVNKVDLETMMPLKTIGLHHHGCVPQAMATHLGGYFF |
| NOV17j | IPPTNLIINHIRFGFIFNKSDPAVHKVDLETMMPLKTIGLHHHGCVPQAMATHLGGYFF |
| NOV17k | IPPTNLIINHIRFGFIFNKSDPAVHKVDLETMMPLKTIGLHHHGCVPQAMAHTHLGGYFF |
| NOV17l | ------------------------------------------------------------ |
| NOV17m | CHASGHEQLFQTHVLQVNVPPVIRVYPESQAQEPGVAASLRCHAEGIPMPRITWLKNGVD |
| NOV17n | CHASGUEQLFQTHVLQVNVPPVIRVYPESQAQEPGVAASLRCHAEGIPMPRITWLKNGVD |
| NOV17o | ------------------------------------------------------------ |
| NOV17p | CHASGHEQLFQTHVLQVNVPPVIRVYPESQAQEPGVAASLRCHAEGIPMPRITWLKNGVD |
| NOV17q | CHASGHEQLFQTHVLQVNVPPVIRVYPESQAQEPGVAASLRCHAEGIPMPRITWLKNGVD |
| NOV17a | VSTQMSKQLSLLANGSELHISSVRYEDTGAYTCIAKNEVGVDEDISSLFIEDSARKTLAN |
| NOV17b | VSTQMSKQLSLLANGSELHISSVRYEDTGAYTCIAKNEVGVDEDISSLFIEDSARKTLAN |
| NOV17c | VSTQMSKQLSLLANGSELHISSVRYEDTGAYTCIAKNEVGVDEDISSLFIEDSARKTRLS |
| NOV17d | IQCRQDSPASAARQLLVDSVTDSVLG---------------------------------- |
| NOV17e | VSTQMSKQLSLLANGSE------------------------------------------- |
| NOV17f | VSTQMSKQLSLLANGSELHISSVRYEDTGAYTCIAKNEVGVDEDISSLFIEDSARKTLAN |
| NOV17g | VSTQMSKQLSLLANGSE------------------------------------------- |
| NOV17h | VSTQMSKQLSLLANGSE------------------------------------------- |
| NOV17i | IQCRQDSPASAARQLLVDSVTDSVLG---------------------------------- |
| NOV17j | IQCRQDSPASAARQLLVDSVTDSVLG---------------------------------- |
| NOV17k | IQCRQDSPASAARQLLVDSVTDSVLG---------------------------------- |
| NOV17l | ------------------------------------------------------------ |
| NOV17m | VSTQMSKQLSLLANGSELHISSVRYEDTGAYTCIAKNEVGVDEDISSLFIEDSARKTLAN |
| NOV17n | VSTQMSKQLSLLANGSE------------------------------------------- |
| NOV17o | ------------------------------------------------------------ |
| NOV17p | VSTQMSKQLSLLANGSELHISSVRYEDTGAYTCIAKNEVGVDEDISSLFIEDSARKTLAN |
| NOV17q | VSTQMSKQLSLLANGSELHISSVRYEDTGAYTCIAKNEVGVDEDISSLFIEDSARKTLAN |
| NOV17a | ILWREEGLSVGNMFYVFSDDGIIVIHPVDCEIQRHLKPTEKIFMSYEEICPQREKNATQP |
| NOV17b | ILWREEGLSVGNMFYVFSDDGIIVIHPVDCEIQRHLKPTEKIFMSYEEICPQREKNATQP |
| NOV17c | VGNMFYVFS---------DDGIIVIHPVDCETQRHLKPTEKIFMSYEEICPQREKNATQP |
| NOV17d | ------------------------------------------------------------ |
| NOV17e | ------------------------------------------------------------ |
| NOV17f | ILWREEGLSVGNMFYVFSDDGIIVIHPVDCEIQRHLKPTEKIFMSYEEICPQREKNATQP |
| NOV17g | ------------------------------------------------------------ |
| NOV17h | ------------------------------------------------------------ |
| NOV17i | ------------------------------------------------------------ |
| NOV17j | ------------------------------------------------------------ |
| NOV17k | ------------------------------------------------------------ |
| NOV17l | ------------------------------------------------------------ |
| NOV17m | ILWREEGLSVGNMFYVFSDDGIIVIHPVDCEIQRHLKPTEKIFMSYEEICPQREKNATQP |
| NOV17n | ------------------------------------------------------------ |
| NOV17o | ------------------------------------------------------------ |
| NOV17p | ILWREEGLSVGNMFYVFSDDGIIVIHPVDCEIQRHLKPTEKIFMSYEEICPQREKNATQP |
| NOV17q | ILWREEGLSVGNMFYVFSDDGIIVIHPVDCEIQRHLKPTEKIFMSYEEICPQREKNATQP |
| NOV17a | CQWVSAVNVRNRYIYVAQPALSRVLVVDIQAQKVLQSIGVDPLPAKLSYDKSHDQVWVLS |
| NOV17b | CQWVSAVNVRNRYIYVAQPALSRVLVVDIQAQKVLQSIGVDPLPAKLSYDKSHDQVWVLS |
| NOV17c | CQWVSAVNVRNRYIYVAQPALSRVLVVDIQAQKVLQSIGVDPLPAKLSYDKSHDQVWVLS |
| NOV17d | ------------------------------------------------------------ |
| NOV17e | ------------------------------------------------------------ |
| NOV17f | CQWVSAVNVRNRYIYVAQPALSRVLVVDIQAQKVLQSIGVDPLPAKLSYDKSHDQVWVLS |
| NOV17g | ------------------------------------------------------------ |
| NOV17h | ------------------------------------------------------------ |
| NOV17i | ------------------------------------------------------------ |
| NOV17j | ------------------------------------------------------------ |
| NOV17k | ------------------------------------------------------------ |
| N0V17l | ------------------------------------------------------------ |
| NOV17m | CQWVSAVNVRNRYIYVAQPALSRVLVVDIQAQKVLQSIGVDPLPAKLSYDKSHDQVWVLS |
| NOV17n | ------------------------------------------------------------ |
| NOV17o | ------------------------------------------------------------ |
| NOV17p | CQWVSAVNVRNRYIYVAQPALSRVLVVDIQAQKVLQSIGVDPLPAKLSYDKSHDQVWVLS |
| NOV17q | CQWVSAVNVRNRYIYVAQPALSRVLVVDIQAQKVLQSIGVDPLPAKLSYDKSHDQVWVLS |
| NOV17a | WGDVHKSRPSLQVITEASTGQSQHLIRTPFAGVDDFFIPPTNLIINHIRFGFIFNKSDPA |
| NOV17b | WGDVHKSRPSLQVITEASTGQSQHLIRTPFAGVDDFFIPPTNLIINHIRFGFIFNKSDPA |
| NOV17c | WGDVHKSRPSLQVITEASTGQSQHLIRTPFACVDDFFIPPTNLIINHIRFGFIFNKSDPA |
| NOV17d | ------------------------------------------------------------ |
| NOV17e | ------------------------------------------------------------ |
| NOV17f | WGDVHKSRPSLQVITEASTGQSQHLIRTPFAGVDDFFIPPTNLIINHIRFGFIFNKSDPA |
| NOV17g | ------------------------------------------------------------ |
| NOV17h | ------------------------------------------------------------ |
| NOV17i | ------------------------------------------------------------ |
| NOV17j | ------------------------------------------------------------ |
| NOV17k | ------------------------------------------------------------ |
| NOV17l | ------------------------------------------------------------ |
| NOV17m | WGDVHKSRFSLQVITEASTGQSQHLIRTPFAGVDDFFIPPTNLIINHIRFGFIFNKSDPA |
| NOV17n | ------------------------------------------------------------ |
| NOV17o | ------------------------------------------------------------ |
| NOV17p | WGDVHKSRPSLQVITEASTGQSQHLIRTPFAGVDDFFIPPTNLIINNIRFGFIFNKSDPA |
| NOV17q | WGDVHKSRPSLQVITEASTGQSQHLILTPFAGVDDFFIPPTNLIINNIRFGFIFNKSDPA |
| NOV17a | VHKVDLETMMPLKTIGLHHHGCVPQANAHTHLGGYFPIQCRQDSPASAARQLLVDSVTDS |
| NOV17b | VHKVDLETMMPLKTIGLHHHGCVPQANAHTHLGGYFFIQCRQDSPASAARQLLVDSVTDS |
| NOVl7c | VRKVDLETMNPLKTIGLHHHGCVPQANAHTHLGGYFFIQCRQDSPASAARQLLVDSVTDS |
| NOV17d | ------------------------------------------------------------ |
| NOV17e | ------------------------------------------------------------ |
| NOV17f | VNKVDLETMMPLKTIGLHHHGCVPQANAHTHLGGYFFIQCRQDSPASAARQLLVDSVTDS |
| NOV17g | ------------------------------------------------------------ |
| NOV17h | ------------------------------------------------------------ |
| NOV17i | ------------------------------------------------------------ |
| NOV17j | ------------------------------------------------------------ |
| NOV17k | ------------------------------------------------------------ |
| NOV17l | ------------------------------------------------------------ |
| NOV17m | VHKVDLETMMPLKTIGLHHHGCVPQANAHTHLGGYFFIQCRQDSPASAARQLLVDSVTDS |
| NOV17n | ------------------------------------------------------------ |
| NOV17o | ------------------------------------------------------------ |
| NOV17p | VHKVDLETMMPLKTIGLHHHGCVPQANAHTHLGGYFFIQCRQDSPASAARQLLVDSVTDS |
| NOV17q | VHKVDLETMMPLKTIGLHHHGCVPQANAHTHLGGYFFIQCRQDSPASAARQLLVDSVTDS |
| NOV17a | VLGPNGDVTGTPHTSPDGRFIVSAAADSPWLHVQEITVRGEIQTLYDLQINSGISDLAFQ |
| NOV17b | VLGPNGDVTGTPHTSPDGRFIVSAAADSPWLHVQEITVROEIQTLYDLQINSGISDLAFQ |
| NOV17c | VLGPNGDVTGTPHTSPOGRPIVSAAADSPWLHVQEITVRGEIQTLYDLQINSGISDLAFQ |
| NOV17d | ---PNGDVTGTPHTSPDORFIVSAAADSPWLHVQEITVRGEIQTLYDLQINSGISDLAFQ |
| NOV17e | ------------------------------------------------------------ |
| NOV17f | VLGPNGDVTGTPHTSPDGRFIVSAAADSPWLHVQEITVRGEIQTLYDLQINSGISDLAFQ |
| NOV17g | ------------------------------------------------------------ |
| NOV17h | ------------------------------------------------------------ |
| NOV17i | ---PNGDVTGTPHTSPDGRFIVSAAADSPWLHVQEITVRGEIQTLYDLQINSGISDLAFQ |
| NOV17j | ---PNGDVTGTPHTSPDGRFIVSAAADSPWLHVQEITVRGEIQTLYDLQINSGISDLAFQ |
| NOV17k | ---PNGDVTGTPHTSPDGRFIVSAAAOSPWLHVQEITVRGEIQTLYDLQINSGISDLAFQ |
| NOV17l | ------------------------------------------------------------ |
| NOV17m | VLGPNGDVTGTPHTSPDGRFIVSAAADSPWLHVQEITVRGEIQTLYDLQINSGISDLAFQ |
| NOV17n | ------------------------------------------------------------ |
| NOV17o | ------------------------------------------------------------ |
| NOV17p | VLGPNGDVTGTPHTSPDORFIVSAAADSPWLHVQEITVRGEIQTLYDLQINSGISDLAFQ |
| NOV17q | VLGPNGDVTGTPHTSPDGRFIVSAAADSPWLHVQEITVRGEIQTLYDLQINSGISDLAFQ |
| NOV17a | RSFTESNQYNIYAALHTEPDLLFLELSTGKVGMLKNLKEPPAGPAQPWGGTHRIMRDSGL |
| NOV17b | RSFTESNQYNIYAALHTEPDLLFLELSTGKVGMLKNLKEPPAGPAQPWGGTHRIMRDSGL |
| NOV17c | RSFTESNQYNIYAALHTEPDLLFLELSTGKVGMLKNLKEPPAGPAQPWGGTNRIMRDSGL |
| NOV17d | RSFTESNQYNIYAALHNEPDLLFLELSTGKVGMLKNLKEPPAGPAQPWGOTHRIMRDSGL |
| NOV17e | ----------------------------------------------------LHISSVRY |
| NOV17f | RSFTESNQYNIYAALNMEPDLLFLELSTGKVGMLKNLKEPPAGPAQPWGGTHRIMRDSGL |
| NOV17g | ----------------------------------------------------LHISSVRY |
| NOV17h | ----------------------------------------------------LHISSVRY |
| NOV17i | RSFTESNQYNIYAALHTEPDLLFLELSTGKVGMLKNLKEPPAGPAQPWGGTHRIMRDSGL |
| NOV17j | RSFTESNQYNIYAALHTEPDLLFLELSTGKVGMLKNLKEPPAGPAQPWGGTNRIMRDSGL |
| NOV17k | RSFTESNQYNIYAALHTEPDLLFLELSTGKVGMLKNLKEPPAGPAQPWGGTHRIMRDSGL |
| NOV17l | ------------------------------------------------------------ |
| NOV17m | RSFTESNQYNIYAALHTEPDLLFLELSTGKVGMLKNLKEPPAGPAQPWGGTHRIMRDSGL |
| NOV17n | ----------------------------------------------------LHISSVRY |
| NOV17o | ------------------------------------------------------------ |
| NOV17p | RSFTESNQYNIYAALHTEPDLLFLELSTGKVGMLKNLKEPPAGPAQPWGGTHRIMRDSGL |
| NOV17q | RSFTESNQYNIYAALHTEPDLLFLELSTGKVGMLKNLKEPPAGPAQPRGGTHRIMRDSGL |
| NOV17a | FGQYLLTPARESLFLINGRQNTLRCEVSGIKGGTTVVWVGEV------------------ |
| NOV17b | FGQYLLTPARESLFLINGRQNTLRCEVSGIKGGTTVVWVGEVLE---------------- |
| NOV17c | FGQYLLTPARESLFLINGRQNTLRCEVSGIKGGTTVVWVGEVLE---------------- |
| NOV17d | FGQYLLTFARESLFLINGRQNTLRCEVSGIKGGTTVVWVGEVLE---------------- |
| NOV17e | EDTOAYTCIAKNEVGVDEDISSLFIEDSARKTLANILWREEGLSVGNMFYVFSDDGIIVI |
| NOV17f | FGQYLLTPARESLFLINGRQNTLRCEVSGIKGGTTVTVVWVGEVLE-------------- |
| NOV17g | EDTGAYTCIAKNEVOVDEDISSLFIEDSARKTLANILWREEGLSVGNMFYVFSDDGIIVI |
| NOV17h | EDTGAYTCIAKNEVGVDEDISSLFIEDSARKTLANILWREEGLSVGNMFYVFSDDGIIVI |
| NOV17i | FGQYLLTPARESLFLINGRQNTLRCEVSGIKGGTTVVWLE-------------------- |
| NOV17j | FGQYLLTPARESLFLINGRQNTLRCEVSCIKGGTTVVWLE-------------------- |
| NOV17k | FGQYLLTPARESLFLINGRQNTLRCEVSGIKOGTTVVWLE-------------------- |
| N0V17l | -------------------DSLYITKVTTIHMGNYTCHASGHEQLFQTHVLQVNVPPVIR |
| NOV17m | FGQYLLTPARESLFLINGRQNTLRCEVSGIKGGTTVVWVGEVEFG--------------- |
| NOV17n | EDTGAYTCIAKNEVGVDEDISSLFIEDSARKTLANILWREEGTKLHCFASCL-------- |
| NOV17o | -------------------QNTLRCEVSGIKGGTTVVWVGEV------------------ |
| NOV17p | FGQYLLTFARESLFLINCRQNTLRCEVSGIKGGTTVVWVGEV------------------ |
| NOV17q | FGQYLLTPARESLFLINGRQNTLRCEVSGIKGGTTVVWVGEV------------------ |
| NOV17a | ------------------------------------------------------------ |
| NOV17b | ------------------------------------------------------------ |
| NOV17c | ------------------------------------------------------------ |
| NOV17d | ------------------------------------------------------------ |
| NOV17e | ------------------------------------------------------------ |
| NOV17f | ------------------------------------------------------------ |
| NOV17g | HPVDCEIQRHLKPTEKIFMSYEEICPQREKNATQPCQWVSAVRNRYIYVAQPALSRVL |
| NOV17h | HPVDCEIQRHLKFTEKIFMSYEEICPQREKNATQPCQWVSAVRNRYIYVAQPALSRVL |
| NOV17i | ---------------------------------------------------------- |
| NOV17j | ---------------------------------------------------------- |
| NOV17k | ---------------------------------------------------------- |
| NOV17l | WPESQAQEFGVAASLRCHAEGIPMPRITWLKNGVDVSTQMSKQLSLLANGSELHISSVR |
| NOV17m | ----------------------------------------------------------- |
| NOV17n | ---------------------------------------------------------- |
| NOV17o | ---------------------------------------------------------- |
| NOV17p | ---------------------------------------------------------- |
| NOV17q | ---------------------------------------------------------- |
| NOV17a | ------------------------------------------------------------ |
| NOV17b | ------------------------------------------------------------ |
| NOV17c | ------------------------------------------------------------ |
| NOV17d | ------------------------------------------------------------ |
| NOV17e | VVDIQAQKVLQSIGVDPLPAKLSYDKSHDQVWVLSWGDVHKSRPSLQVITEASTGQSQHL |
| NOV17f | ------------------------------------------------------------ |
| NOV17g | VVDIQAQKVLQSIGVDPLPAKLSYDKSHDQVWVLSWGDVHKSRPSLQVITEASTGQSQHL |
| NOV17h | VVDIQAQKVLQSIGVDPLPAKLSYDKSHDQVWVLSWGDVHKSRPSLQVITEASTGQSQHL |
| NOV17i | ------------------------------------------------------------ |
| NOV17j | ------------------------------------------------------------ |
| NOV17k | ------------------------------------------------------------ |
| NOV17l | YEDTGAYTCIAKNEVGVDEDISSLFIEDSARKTLANILWREEGLSVGNMFYVFSDDGIIV |
| NOV17m | ------------------------------------------------------------ |
| NOV17n | ------------------------------------------------------------ |
| NOV17o | ------------------------------------------------------------ |
| NOV17p | ------------------------------------------------------------ |
| NOV17q | ------------------------------------------------------------ |
| NOV17a | ------------------------------------------------------------ |
| NOV17b | ------------------------------------------------------------ |
| NOV17c | ------------------------------------------------------------ |
| NOV17d | ------------------------------------------------------------ |
| NOV17e | IRTPFAGVDDFFIPPTNLIINHIRFGFIFNKSDPTVNKVDLETMMPLKTIGLHHNGCVPQ |
| NOV17f | ------------------------------------------------------------ |
| NOV17g | IRTPFAGVDDFFIPPTNLIINHIRFGFIFNKSDPAVHKVDLETMMPLKTIGLHHHGCVPQ |
| NOV17h | IRTPFAGVDDFFIPPTNLIINHIRFGFIFNKSDPAVHKVDLETMMPLKTIGLHHHGCVPQ |
| NOV17i | ------------------------------------------------------------ |
| NOV17j | ------------------------------------------------------------ |
| NOV17k | ------------------------------------------------------------ |
| NOV17l | IHPVDCEIQRHLKPTEKIFNSYEEICPQREKNATQPCQWVSAVNVRNRYIYVAQPALSRV |
| NOV17m | ------------------------------------------------------------ |
| NOV17n | ------------------------------------------------------------ |
| NOV17o | ------------------------------------------------------------ |
| NOV17p | ------------------------------------------------------------ |
| NOV17q | ------------------------------------------------------------ |
| NOV17a | ------------------------------------------------------------ |
| NOV17b | ------------------------------------------------------------ |
| NOV17c | ------------------------------------------------------------ |
| NOV17d | ------------------------------------------------------------ |
| NOV17e | AMAHTHLGGYFFIQCRQDSPASAARQLLVDSVTDSVLGPNGDVTGTPHTSPDGRFIVSAA |
| NOV17f | ------------------------------------------------------------ |
| NOV17g | AMAHTHLOGYFFIQCRQDSPASAARQLLVDSVTDSVLGPNGDVTCTPHTSPDGRFIVSAA |
| NOV17h | AMAHTHLGGYFFIQCRQDSPASAARQLLVDSVTDSVLGPNGDVTGTPHTSPDGRFIVSAA |
| NOV17i | ------------------------------------------------------------ |
| NOV17j | ------------------------------------------------------------ |
| NOV17k | ------------------------------------------------------------ |
| NOV17l | LVVDIQAQKVLQSIGVDPLPAKLSYDKSHDQVWVLSWGDVHKSRPSLQVITEASTGQSQH |
| NOV17m | ------------------------------------------------------------ |
| NOV17n | ------------------------------------------------------------ |
| NOV17o | ------------------------------------------------------------ |
| NOV17p | ------------------------------------------------------------ |
| NOV17q | ------------------------------------------------------------ |
| NOV17a | ------------------------------------------------------------ |
| NOV17b | ------------------------------------------------------------ |
| NOV17c | ------------------------------------------------------------ |
| NOV17d | ------------------------------------------------------------ |
| NOV17e | ADSPWLHVQEITVRCEIQTLYDLQINSGISDLAFQRSFTESNQYNIYAALHTEPDLLFLE |
| NOV17f | ------------------------------------------------------------ |
| NOV17g | ADSPWLHVQEITVRGEIQTLYDLQINSGISDLAFQRSFTESNQYNIYAALHTEPDLLFLE |
| NOV17h | ADSPWLHVQEITVRGEIQTLYDLQINSGISDLAFQRSFTESNQYNIYAALHTEPDLLFLE |
| NOV17i | ------------------------------------------------------------ |
| NOV17j | ------------------------------------------------------------ |
| NOV17k | ------------------------------------------------------------ |
| NOV17l | LIRTPFAGVDDFFIPPTNLIINHIRFGFIFNKSEPAVHKVDLETMAPLKTIGLHHHGCVP |
| NOV17m | ------------------------------------------------------------ |
| NOV17n | ------------------------------------------------------------ |
| NOV17o | ------------------------------------------------------------ |
| NOV17p | ------------------------------------------------------------ |
| NOV17q | ------------------------------------------------------------ |
| NOV17a | ------------------------------------------------------------ |
| NOV17b | ------------------------------------------------------------ |
| NOV17c | ------------------------------------------------------------ |
| NOV17d | ------------------------------------------------------------ |
| NOV17e | LSTGKVGMLKNLKEPPAGPAQPWGGTHRIMRDSGLFGQYLLTPARESLFLINGRQNTLRC |
| NOV17f | ------------------------------------------------------------ |
| NOV17g | LSTGKVGMLKNLKEPPAGPAQPWGGTHRIMRDSGLFGQYLLTPARESLFLINGRQNTLRC |
| NOV17h | LSTGKVGMLKNLKEPPAGPAQPWGCTHRIMRDSGLFGQYLLTPARESLFLINGRQNTLRC |
| NOV17i | ------------------------------------------------------------ |
| NOV17j | ------------------------------------------------------------ |
| NOV17k | ------------------------------------------------------------ |
| NOV17l | QAMARTULGGYFFIQCRQDSPASAARQLLVDSVTDSVLGPNGDVTGTPHTSPDGRFIVSA |
| NOV17m | ------------------------------------------------------------ |
| NOV17n | ------------------------------------------------------------ |
| NOV17o | ------------------------------------------------------------ |
| NOV17p | ------------------------------------------------------------ |
| NOV17q | ------------------------------------------------------------ |
| NOV17a | ------------------------------------------------------------ |
| NOV17b | ------------------------------------------------------------ |
| NOV17c | ------------------------------------------------------------ |
| NOV17d | ------------------------------------------------------------ |
| NOV17e | EVSGIKGGTTVVWVGEVLE----------------------------------------- |
| NOV17f | ------------------------------------------------------------ |
| NOV17g | EVSGIKGGTTVVWVGEV------------------------------------------- |
| NOV17h | EVSGIKCGTTVVWVGEVEFG---------------------------------------- |
| NOV17i | ------------------------------------------------------------ |
| NOV17j | ------------------------------------------------------------ |
| NOV17k | ------------------------------------------------------------ |
| NOV17l | AADSPWLHVQEITVRGEIQTLYDLQINSGISDLAFQRSFTESNQYNIYAALHTEPDLLFL |
| NOV17m | ------------------------------------------------------------ |
| NOV17n | ------------------------------------------------------------ |
| NOV17o | ------------------------------------------------------------ |
| NOV17p | ------------------------------------------------------------ |
| NOV17q | ------------------------------------------------------------ |
| NOV17a | ------------------------------------------------------------ |
| NOV17b | ------------------------------------------------------------ |
| NOV17c | ------------------------------------------------------------ |
| NOV17d | ------------------------------------------------------------ |
| NOV17e | ------------------------------------------------------------ |
| NOV17f | ------------------------------------------------------------ |
| NOV17g | ------------------------------------------------------------ |
| NOV17h | ------------------------------------------------------------ |
| NOV17i | ------------------------------------------------------------ |
| NOV17j | ------------------------------------------------------------ |
| NOV17k | ------------------------------------------------------------ |
| NOV17l | ELSTGKVGMLKNLKEPPAGPAQPRCGTHRIMRDSGLFGQYLLTPARESLFLINGRQNTLR |
| NOV17m | ------------------------------------------------------------ |
| NOV17n | ------------------------------------------------------------ |
| NOV17o | ------------------------------------------------------------ |
| NOV17p | ------------------------------------------------------------ |
| NOV17q | ------------------------------------------------------------ |
| NOV17a | --------------------- |
| NOV17b | --------------------- |
| NOV17c | --------------------- |
| NOV17d | --------------------- |
| NOV17e | --------------------- |
| NOV17f | --------------------- |
| NOV17g | --------------------- |
| NOV17h | --------------------- |
| NOV17i | --------------------- |
| NOV17j | --------------------- |
| NOV17k | --------------------- |
| NOV17l | CEVSGIKGGTTVVWVGEVEFG |
| NOV17m | --------------------- |
| NOV17n | --------------------- |
| NOV17o | --------------------- |
| NOV17p | --------------------- |
| NOV17q | --------------------- |
| NOV17a | (SEQ ID NO: 240) |
| NOV17b | (SEQ ID NO: 242) |
| NOV17c | (SEQ ID NO: 244) |
| NOV17d | (SEQ ID NO: 246) |
| NOV17e | (SEQ ID NO: 248) |
| NOV17f | (SEQ ID NO: 250) |
| NOV17g | (SEQ ID NO: 252) |
| NOV17h | (SEQ ID NO: 254) |
| NOV17i | (SEQ ID NO: 256) |
| NOV17j | (SEQ ID NO: 258) |
| NOV17k | (SEQ ID NO: 260) |
| N0V17l | (SEQ ID NO: 262) |
| NOV17m | (SEQ ID NO: 264) |
| NOV17n | (SEQ ID NO: 266) |
| NOV17o | (SEQ ID NO: 268) |
| NOV17p | (SEQ ID NO: 270) |
| NOV17q | (SEQ ID NO: 272) |

Further analysis of the NOV17a protein yielded the following properties shown in Table 17C.

TABLE 17C
Protein Sequence Properties NOV17a

| SignalP | Cleavage site between residues 23 and 24 | | | |
|---|---|---|---|---|
| analysis: | | | | |
| PSORT II | PSG: a new signal peptide prediction method | | | |
| analysis: | N-region: length 2; pos. chg 1; neg. chg 0 | | | |
| | H-region: length 22; peak value 8.15 | | | |
| | PSG score: 3.75 | | | |
| | GvH: von Heijne's method for signal seq. recognition | | | |
| | GvH score (threshold: −2.1): 2.14 | | | |
| | possible cleavage site: between 22 and 23 | | | |
| | >>> Seems to have a cleavable signal peptide (1 to 22) | | | |
| | ALOM: Klein et al's method for TM region allocation | | | |
| | Init position for calculation: 23 | | | |
| | Tentative number of TMS(s) for the threshold 0.5: 1 | | | |
| | Number of TMS(s) for threshold 0.5: 0 | | | |
| | PERIPHERAL | Likelihood = | 3.39 (at 513) | |
| | ALOM score: −0.27 (number of TMSs: 0) | | | |
| | MTOP: Prediction of membrane topology (Hartmann et al.) | | | |
| | Center position for calculation: 11 | | | |
| | Charge difference: −4.5 C(−2.0)-N(2.5) | | | |
| | N >= C: N-terminal side will be inside | | | |
| | MITDISC: discrimination of mitochondrial targeting seq | | | |
| | R content: | 0 | Hyd Moment(75): | 3.60 |
| | Hyd Moment(95): | 5.25 | G content: | 4 |
| | D/E content: | 1 | S/T content: | 2 |

Score: −6.75
Gavel: prediction of cleavage sites for mitochondrial preseq
cleavage site motif not found
NUCDISC: discrimination of nuclear localization signals
pat4: none
pat7: none
bipartite: none
content of basic residues: 9.1%
NLS Score: −0.47
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 76.7
COIL: Lupas's algorithm to detect coiled-coil regions
total: 0 residues
Final Results (k = 9/23):
33.3%: extracellular, including cell wall
22.2%: vacuolar
22.2%: mitochondrial
22.2%: endoplasmic reticulum
>> prediction for CG52643-02 is exc (k = 9)

A search of the NOV17a protein against the Geneseq database, a proprietary database that contains sequences published in patents and patent publication, yielded several homologous proteins shown in Table 17D.

TABLE 17D
Geneseq Results for NOV17a

| | | NOV17a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAB82300 | Human follistatin-related protein | 1 . . . 842 | 842/842 (100%) | 0.0 |
| | zfsta4 - Homo sapiens, 842 aa. | 1 . . . 842 | 842/842 (100%) | |
| | [WO200132871-A2, 10 MAY | | | |
| | 2001] | | | |
| AAB19727 | Human SECX Clone 4324229-2 | 1 . . . 842 | 842/842 (100%) | 0.0 |
| | encoded protein - Homo sapiens, | 1 . . . 842 | 842/842 (100%) | |
| | 842 aa. [WO200061754-A2, 19 | | | |
| | OCT. 2000] | | | |
| AAM79505 | Human protein SEQ ID NO 3151 - | 5 . . . 842 | 837/838 (99%) | 0.0 |
| | Homo sapiens, 838 aa. | 1 . . . 838 | 837/838 (99%) | |
| | [WO200157190-A2, 09 AUG. | | | |
| | 2001] | | | |
| AAM78521 | Human protein SEQ ID NO 1183 - | 24 . . . 842 | 819/819 (100%) | 0.0 |
| | Homo sapiens, 819 aa. | 1 . . . 819 | 819/819 (100%) | |
| | [WO200157190-A2, 09 AUG. | | | |
| | 2001] | | | |
| AAU08678 | Human FCTR2 polypeptide | 54 . . . 842 | 788/789 (99%) | 0.0 |
| | sequence - Homo sapiens, 815 aa. | 27 . . . 815 | 788/789 (99%) | |
| | [WO200166747-A2, 13 SEP. 2001] | | | |

In a BLAST search of public sequence databases, the NOV17a protein was found to have homology to the proteins shown in the BLASTP data in Table 17E.

TABLE 17E
Public BLASTP Results for NOV17a

| | | NOV17a | Identities/ | |
|---|---|---|---|---|
| Protein | | Residues/ | Similarities for | |
| Accession | | Match | the Matched | Expect |
| Number | Protein/Organism/Length | Residues | Portion | Value |
| CAC39534 | Sequence 1 from Patent | 1 . . . 842 | 842/842 (100%) | 0.0 |
| | WO0132871 - Homo sapiens | 1 . . . 842 | 842/842 (100%) | |
| | (Human), 842 aa. | | | |
| CAC88673 | Sequence 3 from Patent | 54 . . . 842 | 788/789 (99%) | 0.0 |
| | WO0166747 - Homo sapiens | 27 . . . 815 | 788/789 (99%) | |
| | (Human), 815 aa. | | | |
| Q9UPU1 | Hypothetical protein KIAA1061 - | 150 . . . 842 | 693/693 (100%) | 0.0 |
| | Homo sapiens (Human), 693 aa | 1 . . . 693 | 693/693 (100%) | |
| | (fragment). | | | |
| Q8TBU0 | Similar to KIAA1061 protein - | 1 . . . 603 | 601/603 (99%) | 0.0 |
| | Homo sapiens (Human), 605 aa. | 1 . . . 603 | 602/603 (99%) | |
| Q8N475 | Hypothetical protein | 42 . . . 841 | 470/806 (58%) | 0.0 |
| | DKFZp566D234 - Homo sapiens | 42 . . . 846 | 617/806 (76%) | |
| | (Human), 847 aa. | | | |

PFam analysis predicts that the NOV17a protein contains the domains shown in the Table 17F.

TABLE 17F
Domain Analysis of NOV17a

| | | Identities/ | |
|---|---|---|---|
| | | Similarities | |
| Pfam | NOV17a Match | for the | Expect |
| Domain | Region | Matched Region | Value |
| kazal | 89 . . . 133 | 21/61 (34%) | 3.4e−09 |
| | | 30/61 (49%) | |
| efhand | 178 . . . 206 | 8/29 (28%) | 0.065 |
| | | 21/29 (72%) | |
| ig | 263 . . . 323 | 22/64 (34%) | 0.0056 |
| | | 40/64 (62%) | |
| ig | 355 . . . 415 | 21/64 (33%) | 2e−08 |
| | | 46/64 (72%) | |

### Example 18

The NOV18 clone was analyzed, and the nucleotide and encoded polypeptide sequences are shown in Table 18A.

TABLE 18A
NOV18 Sequence Analysis

| | | |
|---|---|---|
| NOV18a, CG53270-01 | SEQ ID NO: 279 | 1140 bp |
| DNA Sequence | ORF Start: ATG at 21 | ORF Stop: TGA at 1122 |

CACTGGGCATTCCTGGCACCATGCATGACGCTGCTGTCCTCAAGCGACGAGGCTACCTCCTGGGGATA
AATTTAGGAGAGGGCTCCTATGCAAAAGTAAAATCTGCTTACTCTGAGCGCCTGAAGTTCAATGTGGC
GATCAAGATCATCGACCGCAAGAAGGCCCCCGCAGACTTCTTGGAGAAATTCCTTCCCCGGGAAATTG
AGATTCTGGCCATGTTAAACCACTGCTCCATCATTAAGACCTACGAGATCTTTGAGACATCACATGGC
AAGGTCTACATCGTCATGGAGCTCGCGGTCCAGGGCGACCTCCTCGAGTTAATCAAAACCCGGGGAGC
CCTGCATGAGGACGAAGCTCGCAAGAAGTTCCACCAGCTTTCCTTGGCCATCAAGTACTGCCACGACC
TGGACGTCGTCCACCGGGACCTCAGTGTGACAACCTTCTCCTTGACAAGGACTTCAACATCAAGCTG
TCCGACTTCAGCTTCTCCAAGCGCTGCCTGCGGGATGACAGTGGTCGCATGGCATTAAGCAAGACCTT
CTGTGGGTCACCACCGTATGCGGCCCCAGAGGTGCTGCAGGGCATTCCCTACCAGCCCAAGGTGTACG
ACATCTGGAGCCTAGGCGTGATCCTCTACATCATGGTCTGCGGCTCCATGCCCTACGACGACTCCAAC
ATCAAGAAGATGCTGCGTATCCAGAAGGAGCACCGCGTCAACTTCCCACGCTCCAAGCACCTGACAGG
CGAGTGCAAGGACCTCATCTACCACATGCTGCAGCCCGACGTCAACCGGCGGCTCCACATCGACGAGA
TCCTCAGCCACTCCTGGATGCAGCCCAAGGCACGGGGATCTCCCTCTGTGGCCATCAACAAGGAGGGG
GAGAGTTCCCGGCGAACTGAACCCTTGTGGACCCCCGAACCTGGCTCTGACAAGAAGTCTGCCACCAA
GCTGCAGCCTGAGGGAGAGGCACAGCCCCAGGCACAGCCTGAGACAAAACCCGACGCGACAGCAATGC
AAATGTCCAGGCAGTCGGAGATCCTGGGTTTCCCCAGCAAGCCGTCGACTATGGAGACAGAGGAAGGG
CCCCCCCAACAGCCTCCAGAGACGCGGGCCCAGTGAGCTTCTTGCGGCCCAG

| | | | |
|---|---|---|---|
| NOV18a, CG53270-01 | SEQ ID NO: 280 | 367 aa | MW at 41617.4 kD |
| Protein Sequence | | | |

MDDAAVLKRRGYLLGINLGEGSYAKSAYSERLKFNVAIKIIDRKKAPAPFLEKFLPREIEILAMLN
HCSIIKTYEIFETSHGKVYIVMELAVQGDLLELIKTRGALHEDERKKFHQLSLAIKYCHDLDVHRD
LKCDNLLLDKDFNIKLSDFSFSKRCLRDDSGRMALSKTFCGSPAYAAPEVLQGIPYQPKVYDIWSLGV
ILYIMVCGSMPYDDSNIKKMLRIQKEHRVNFPRSKHLTGECKDLIYHMLQPDNRRLHIDEILSHCWM
QPKARGSPSVAINKEGESSRGTEPLWTPEPGSDKKSATKLEPEGEAQPQAQPETKPEGTAQMSRQSE
ILGFPSKPSTMETEEGPPQQPPETRAQ

| | | |
|---|---|---|
| NOV18b, 274089779 | SEQ ID NO: 281 | 802 bp |
| DNA Sequence | ORE Start: at 2 | ORF Stop: end of sequence |

CACCGGATCCTACCTCCTGGGGATAATTTAGGAGAGGGCTCCTATGCGTAAAATCTGCTTACT
CTGAGCGCCTGAGTTCAATGTGGCGATCAAGATCATCGACCGCGAAGGCCCCCGCAGACTTCTTG
GAGAAATTCCTTCCCCGGGAAATTGAGATTCTGGCCATGTTAAACCACTGCTCCATCATTAAGACCTA
CGAGATCTTTGAGACATCACATGGCAAGGTCTACATCGTCATGGAGCTCGCGGTCCAGGGCGACCTCC
TCGAGTTAATCAAACCCGGGGAGCCCTGCATGAGGACGAACCTCGCAAGAAGTTCCACCAGCTTTCC
TTGGCCATCAAGTACTGCCACGACCTGCACGTCGTCCACCGGCACCTCAAGTGTGACAACCTTCTCCT
TGACAAGGACTTCAACATCAAGCTCTCCGACTTCAGCTTCTCCAAGCGCTGCCTGCGGGATGACAGTG
GTCGAATGGCATTAAGCAAGACCTTCTGTGGGTCACCAGCGTATGCGGCCCCAGAGGTGCTGCAGGGC
ATTCCCTACCAGCCCAAGGTGTACGACATCTGGAGCCTAGGCGTGATCCTCTACATCATGGTCTGCCG
CTCCATGCCCTACGACGACTCcACATCAAGAGATCCTGCGTATCCAGAAGGAGCACCGCGTCAAT
TCCCACGCTCCAAGCACCTGACAGGCGAGTGCAAGGACCTCATCTACCACATGCTGCAGCCCGACGTC
AACCGGCGGCTCCACATCGACGAGATCCTCAGCCACTGCTGGATGGGTACCGGC

| | | | |
|---|---|---|---|
| NOV18b, 274089779 | SEQ ID NO: 282 | 267 aa | MW at 30670.4 kD |
| Protein Sequence | | | |

TGSYLLGINLGEGSYAKVKSAYSERLKFNVAIKIIDRKKAPADFLEKFLPREIEILAMLNHCSIIKTY
EIFETSHGKVYIVMELAVQGDLLELIKTRGALHEDEARKKFHQLSLAIKYCHDLDVVHRDLKCDNLLL
DKDFNIKLSDFSFSKRCLRDDSGRMALSKTFCGSPAYAAPEVLQGIPYQPKVYDIWSLGVILYIMVCG
SMPYDDSNIKKMLRIQKEHRVNFPRSKHLTGECKDLIYHMLQPDVNRRLHIDEILSHCWMCTG

| | | |
|---|---|---|
| NOV18c, CG53270-02 | SEQ ID NO: 283 | 1132 bp |
| DNA Sequence | ORF Start: ATG at 15 | ORF Stop: TGA at 1116 |

GCATTCCTGGCACCATGGATGACGCTGCTGTCCTCAAGCGACGAGGCTACCTCCTGGGGATTTTA
GGAGAGGGCTCCTATGCAAAAGTAAAATCTGCTTACTCTGAGCGCCTGGTTCAAGTGGCGATCAA
GATCATCGACCGCAAGAAGGCCCCCGCAGACTTCTTGGAGAAATTCCTTCCCCGGGAAATTGAGATTC
TGGCCATGTTAAACCACTGCTCCATCATTAAGACCTACGAGATCTTTGAGACATCACATGGCAAGTC
TACATCGTCATCGAGCTCGCGGTCCAGGGCGACCTCCTCGAGTTAATCACCCGGGGAGCCCTCCA
TGAGGACGAAGCTCGCAAGAAGTTCCACCAGCTTTCCTTGGCCATCAAGTACTGCCACGACCTGGACG
TCGTCCACCGGGACCTCAAGTGTGACAACCTTCTCCTTGACAAGGACTTCAACATCAAGCTGTCCGAC
TTCACCTTCTCCAAGCGCTGCCTGCGGGATGACAGTGGTCGAATGGCATTAAGCAAGACCTTCTGTGG
GTCACCAGCGTATGCGGCCCCAGAGGTGCTGCAGGGCATTCCCTACCAGCCCAAGGTGTACGACATCT
GGAGCCTAGGCGTGATCCTCTACATCATGGTCTGCGGCTCCATGCCCTACGACGACTCCAACATCAAG
AAGATGCTGCGTATCCAGAAGGAGCACCGCGTCAACTTCCCACGCTCCAAGCACCTGACAGGCGAGTG
CAAGGACCTCATCTACCACATGCTGCAGCCCGACGTCAACCGGCGGCTCCACATCGACGAGATCCTCA
GCCACTGCTGGATGCAGCCCAAGGCACGGGGATCTCCCTCTGTGGCCATCAACAAGGAGGGGGAGAGT
TCCCGGGGAACTGAACCCTTGTCGACCCCCGACCTGGCTCTGACAAGAGTCTGCCACCAAGCTGGA
GCCTGAGGGAGAGCCACAGCCCCAGGCACAGCCTGAGACAAAACCCGAGGGGACAGCAATGCAAATGT
CCAGGCAGTCGGAGATCCTGGGTTTCCCCAGCAAGCCGTCGACTATGGAGACAGAGGAAGGGCCCCCC
CAACAGCCTCCAGAGACGCGGGCCCAGTGAGCTTCTTGCGGCCC

| | | | |
|---|---|---|---|
| NOV 18c, CG53270-02 | SEQ ID NO: 284 | a367 aa | MW at 41617.4 kD |
| Protein Sequence | | | |

MDDAAVLKRRGYLLGINLGEGSYAKVKSAYSERLKFNVAIKIIDRKKAPADFLEKFLPREIEILAMLN
HCSIIKTYEIFETSHGKVYIVMELAVQGDLLELIKTRGALHEDEARKKFHQLSLAIKYCHDLDVVHRD
LKCDNLLLDKDFNIKLSDFSFSKRCLRDDSGRMALSKTFCGSPAYAAPEVLQGIPYQPKVYDIWSLGV
ILYIMVCGSMPYDDSNIKKMLRIQKEHRVNFPRSKHLTGECKDLIYHMLQPDVNRRLHIDEILSHCWM
QPKARGSPSVAINKEGESSRGTEPLWTPEPCSDKKSATKLEPEGEAQPQAQPETKPEGTAMQMSRQSE
ILGFPSKPSTMETEEGPPQQPPETRAQ

| | | |
|---|---|---|
| NOV18d, 13382344 SNP for | SEQ ID NO: | 1140 bp SNP: 89 T/C |
| cg53270-01 | 285 | |
| DNA Sequence | ORF Start | ORF Stop: end of sequence |
| | ATG at 21 | |

CACTGGGCATTCCTGGCACCATGGATGACGCTGCTGTCCTCAAGCGACGAGGCTACCTCCTGGGGATAAAT
TTAGGAGAGCGCTCCTACGCAAAGTAAAATCTGCTTACTCTGAGCGCCTGAAGTTCAATGTGGCGATCAA
GATCATCGACCGCAAGAGGCCCCCGCAGACTTCTTGGAGAATTCCTTCCCCGGGAATTGAGATTCTGG
CCATGTTAAACCACTGCTCCATCATTAAGACCTACGAGATCTTTGAGACATCACATGGCAAGGTCTACATC
GTCATGGAGCTCGCGGTCCAGGGCGACCTCCTCGAGTTAATCAAACCCGGGGAGCCCTGCATGAGGACGA
AGCTCGCAAGAAGTTCCACCAGCTTTCCTTGGCCATCAAGTACTGCCACGACCTGGACGTCGTCCACCGGG
CGCTGCCTGCGGGATGACAGTGGTCGAATGGCATTAAGCAAGACCTTCTGTGGGTCACCAGCGTATGCGGC
CCCAGAGGTGCTGCAGGGCATTCCCTACCAGCCCAAGGTGTACGACATCTGGAGCCTAGGCGTGATCCTCT
ACATCATGGTCTGCGGCTCCATGCCCTACGACGACTCCAACATCAAGAAGATGCTGCGTATCCAGAAGGAG
CACCGCGTCAACTTCCCACGCTCCAAGCACCTGACAGGCGAGTGCAAGGACCTCATCTACCACATGCTGCA
GCCCGACGTCAACCGGCGGCTCCACATCGACGAGATCCTCAGCCACTGCTGGATGCAGCCCAAGGCACGGG
GATCTCCCTCTGTGGCCATcACAGCAGGGGGAGAGTTCCCGGGGAACTGAACCCTTGTGGACCCCCGAA
CCTGGCTCTGACAAGAAGTCTGCCACCAAGCTGGAGCCTGAGGGAGAGGCACAGCCCCAGGCACAGCCTGA
GACAAAACCCGAGGGGACAGCAATGCAAATGTCCAGGCAGTCGGAGATCCTGGGTTTCCCCAGCAAGCCGT
CGACTATGGAGACAGAGGAAGGGCCCCCCAACAGCCTCCAGAGACGCGGGCCCAGTGAGCTTCTTGCGGC
CCAG

| | | | |
|---|---|---|---|
| NOV18d, 13382344 SNP | SEQ ID NO: | 367 aa | SNP: no change in protein |
| for | 286 | | sequence |
| CG53270-01 | | | |
| Protein Sequence | | | |

MDDAAVLKRRGYLLGINLGEGSYAKVKSAYSERLKFNVAIKIIDRKKAPADFLEKFLPREIEILAMLNHCS
IIKTYEIFETSHGKYIVMELAVQGDLLELIKTRGALHEDEARKKFHQLSLAIKYCHDLDVVHRDLKCDNL
LLDKDFNIKLSDFSFSKRCLRDDSGRMALSKTFCGSPAYAAPEVLQGIPYQPKVYDIWSLGVILYIMVCGS
MPYDDSNIKKNLRIQKEHRVNFPRSKHLTGECKDLIYHMLQPDVNRRLHIDEILSHCWMQPKARGSPSVAI
NKEGESSRGTEPLWTPEPGSDKKSATKLEPEGEAQPQAQPETKPEGTAMQMSRQSEILGFPSKPSTMETEE
GPPQQPPETRAQ

| | | |
|---|---|---|
| NOV18e, 13382345 SNP for | SEQ ID NO: 287 | 1140 bp SNP: 95 A/G |
| CG53270-01 | | |
| DNA Sequence | ORF Stop: TGA at 1122 | |

CACTGGGCATTCCTGGCACCATGGATGACGCTGCTGTCCTCAAGCGACGACGCTACCTCCTGGGGATAAAT
TTAGGAGAGGGCTCCTATGCAAAAGGTTCTGCTTACTCTGAGCGCCTGAAGTTCAAATGTGGCGATCAAA
GATCATCGACCGCAAGAAGGCCCCCGCAGACTTCTTGGAGAATTCCTTCCCCGGGAAATTGAGATTCTGG
CCATGTTAAAAACCACTGCTCCATCATTAAGACCTACGAGATCTTTGAGACATCACATGGCAAGGTCTACATC
GTCATGGAGCTCGCGGTCCAGGGCGACCTCCTCGAGTTAATCAACCCGGGGAGCCCTGCATGAGGACGA
AGCTCGCAAGAAGTTCCACCAGCTTTCCTTGGCCATCAAGTACTGCCACGACCTGGACGTCGTCCACCGGG
CGCTGCCTGCGGGATGACAGTGGTCGAATGGCATTAAGCAAGACCTTCTGTGGGTCACCAGCGTATGCGGC
CCCAGAGGTGCTGCAGGGCATTCCCTACCAGCCCAAGGTGTACGACATCTGGAGCCTAGGCGTGATCCTCT
ACATCATGGTCTGCGGCTCCATGCCCTACGACGACTCCAACATCAAGAAGATGCTGCGTATCCAGAAGGAG
CACCGCGTCAACTTCCCACCCTCCAAGCACCTGACAGGCGAGTGCAAGGACCTCATCTACCACATGCTGCA
GCCCGACGTCAACCGGCGGCTCCACATCGACGAGATCCTCAGCCACTCCTGGATGCAGCCCAAGGCACGGG
GATCTCCCTCTGTGGCCATCAACAAGGAGGGGGAGAGTTCCCGGGGAACTGAACCCTTGTGGACCCCCGAA
CCTGGCTCTGACAAGAAGTCTGCCACCAAAGCTGGAGCCTGAGGGAGAGGCACAGCCCCAGGCACAGCCTGA
GACAAACCCGAGGGGACAGCAATGCAAATGTCCAGGCAGTCGGAGATCCTGGGTTTCCCCAGCAAAGCCGT
CGACTATGGAGACAGAGGAAGGGCCCCCCCAACAGCCTCCAGAGACGCGGGCCCAGTGAGCTTCTTGCGGC
CCAG

| | | | |
|---|---|---|---|
| NOV18e, 13382345 SNP | SEQ ID NO: | 367 aa | SNP: no change in protein |
| for | 288 | sequence | |
| CG53270-01 | | | |
| Protein Sequence | | | |

MDDAAVLKRRGYLLGINLGEGSYAKSAYSERLFNVAIKIIDRKKAPADFLEKEPREIERILAMLNNHCS
IIKTYETFETSHGKVYIVMELAVQGDLLELIKTRGALHEDEARKKFHQLSLATKYCHDLDVVHRDLKCDNL
MPYDDSNIKKMLRIQKEHRVNFRPSKHLTGECKDLIYHLMLQPDVNRRHIDEILSHCWMQPKARGSPSVAI
NKEGESSRGTEPLWTPEPGSDKKSATKLEPEGEAPQAQPTKPEGTAMWQMSRQSEILGFPSKPSTMETEE
GPPQQPPETRAQ

| | | |
|---|---|---|
| NOV18f, 13376391 SNP for | SEQ ID NO: 289 | 1140 bp SNP: 310 A/G |
| CG53270-01 | ORF Start: ATG at 21 | ORF Stop: TGA at 1122 |
| DNA Sequence | | |

CACTGGGCATTCCTGGCACCATGGATGACGCTGCTGTCCTCAAGCGACGAGGCTACCTCCTGGGGATAAAT
TTAGGAGAGGGCTCCTATGCAAAGTTCTGCTTACTCTGAGCGCCTGAAGTTCAATGTGGCGATCAA
GATCATCGACCGCAAGAGGCCCCCGCAGACTTCTTGGAGAATTCCTTCCCCGGGAATTGAGATTCTGG
CCATGTTAAACCACTGCTCCATCATTAAGACCTACGAGATCTTTGAGACATCACATGGCAAGGTCTACATC
GTCATGGAGCTCGCGGTCCAGGGCGGCCTCCTCGAGTTAATCAACCCGGGGAGCCCTGCATGACGACGA
AGCTCGCAAGAAGTTCCACCAGCTTTCCTTGGCCATCAAGTACTGCCACGACCTGGACGTCGTCCACCGGG
CGCTGCCTGCGGGATGACAGTGGTCGAATGGCATTAAGCAAGACCTTCTGTGGGTCACCAGCGTATGCGGC
CCCAGAGGTGCTGCAGGGCATTCCCTACCAGCCCTAAGGTGTACGACATCTGGAGCCTAGGCGTGATCCTCT
CACCGCGTCAACTTCCCACCCTCCAAGCACCTGACAGGCGAGTGCAAGGACCTCATCTACCACATGCTGCA
GCCCGACGTCAACCGGCGGCTCCACATCGACGAGATCCTCAGCCACTGCTGGATGCAGCCCTAAGGCACGGG
CCTGGCTCTGACAAGAAGTCTGCCACCAAGCTGGAGCCTGAGGGAGAGGCACACCCCCAGGCACAGCCTGA
GACAAACCCGAGGGGACAGCAATGCAAATGTCCAGGCAGTCGGAGATCCTGGGTTTCCCCAGCAAGCCGT
CGACTATGGAGACAGAGGAACGGCCCCCCCAACAGCCTCCAGAGACGCGGGCCCAGTGAGCTTCTTGCGGC
CCAG

| | | | |
|---|---|---|---|
| NOV18f, 13376391 SNP for | SEQ ID NO: | 367 aa | SNP: Asp to Gly at position 97 |
| CG53270-01 | 290 | | |
| Protein Sequence | | | |

MDDAAVLKRRGYLLGINLGEGSYAKVKSAYSERLKFBVAIKIIDRKKAPADFLEKFLPREIEILANLNHCS
IIKTYETFETSHGKVYIVMELAVQGGLLELIKTRGALHEDEARKKFHQLSLAIKYCHDLDVVHRDLKCDNL
LLDKDFNISDFSFSCLRDDSGRDIALSKTFCGSPAYPEVLQGIPYQPKVYDIWSLGVILYIMVCGS
MPYDDSNIKKNLRIQKEHRVNFPRSKHLTGECKDLIYHMLQPDVNRRLHIDEILSHCWMQPKARGSPSVAI
NKEGESSRGTEPLWTPEPGSDKKSATKLEPEGEAQPQAPETKPEGTAMQMSRQSEILGFSKPSTMETEE
GPPQQPPETRAQ

| | | |
|---|---|---|
| NOV18g, 13376390 SNP for | SEQ ID NO: 291 | 1140 bp SNP: 978 C/T |
| CG5327001 | ORF Start: ATG at 21 | ORF Stop: TGA at 1122 |
| DNA Sequence | | |

CACTUGGCATTCCTGGCACCATGGATGACGCTGCTGTCCTCAAGCGACGAGGCTACCTCCTGGGGATAAAT
TTAGGAGAGGGCTCCTATGCAAAAGTAAAATCTGCTTACTCTGAGCGCCTGAAGTTCAATGTGGCGATCAA
GATCATCGACCGCAAGAAGGCCCCCGCAGACTTCTTGGAGAAATTCCTTCCCCGGGAAATTGAGATTCTGG
CCATGTTAAACCACTGCTCCATCATTAAGACCTACGAGATCTTTGAGACATCACATGGCAAGGTCTACATC
GTCATGGAGCTCGCGGTCCAGGGCGACCTCCTCGAGTTAATCAAAACCCGGGGAGCCCTGCATGAGGACGA
AGCTCGCAAGAAGTTCCACCAGCTTTCCTTCGCCATCAAGTACTGCCACGACCTGGACGTCGTCCACCGGG
ACCTCAAGTGTGACAACCTTCTCCTTGACAAGGACTTCAACATCAAGCTGTCCGACTTCAGCTTCTCCAAG
CGCTGCCTGCGGGATGACAGTGGTCGAATGGCATTAAGCAAGACCTTCTGTGGGTCACCAGCGTATGCGGC
CCCAGAGGTGCTGCAGGGCATTCCCTACCAGCCCAAGGTGTACGACATCTGGAGCCTAGGCGTGATCCTCT
ACATCATGGTCTGCGGCTCCATGCCCTACGACGACTCCAACATCAACAAGATGCTGCGTATCCAGAAGGAG
CACCGCGTCAACTTCCCACGCTCCAAGCACCTGACAGGCGAGTGCAAGGACCTCATCTACCACATGCTGCA
GCCCGACGTCAACCCGCGGCTCCACATCGACGAGATCCTCAGCCACTGCTGGATGCAGCCCAAGGCACGGG
GATCTCCCTCTGTGGCCATCACAGGAGGGGGAGAGTTCCCGGGGAACTGAACCCTTGTGGACCCCCGAA
CCTGGCTCTGACAAGAAGTCTGCCACCAAGCTGGAGCCTGAGGGAGAGGCACAGTCCCAGGCACAGCCTGA
GACAAAACCCGAGGGGACAGCAATGCAAATGTCCAGGCAGTCGGAGATCCTGGGTTTCCCCAGCAAGCCGT
CGACTATGGAGACAGAGGAAGGGCCCCCCCAACAGCCTCCAGAGACGCGGGCCCAGTGAGCTTCTTGCGGC
CCAG

| | | | |
|---|---|---|---|
| NOV18g, 13376390 SNP for | SEQ ID NO: | 367 aa | SNP: Pro to Ser at position 320 |
| CG53270-01 | 292 | | |
| Protein Sequence | | | |

MDDAAVLKRRGYLLGINLGEGSYAKVKSAYSERLKFNVAIKIIDRKKAPADFLEKFLPREIEILANLNHCS
IIKTYEIFETSHGKVYIVMELAVQGDLLELIKTRGALHEDEARKKFHQLSLAIKYCHDLDVVHRDLKCDNL
LLDKDFNIKLSDFSFSKRCLRDDSGRMALSKTFCGSPAYAAPEVQGIPYQPKVYDIWSLGVILYIMVCGS
MPYDDSNIKKMLRIQKEHRVNFPRSKHLTGECKDLIYHMLQPDVNRRLHIDEILSHCWMQPKARCSPSVAI
NKEGESSRGTEPLWTPEPGSDKKSATKLEPEGEAQSQAQPETKPEGTAMQMSRQSEILGFPSKPSTMETEE
GPPQQPPETRAQ

| | | |
|---|---|---|
| NOV18h, 13376389 SNP for | SEQ ID NO: 293 | 1140 bp SNP: 996 A/G |
| CG53270-01 | ORF Start: ATG at 21 | ORF Stop: TGA at 1122 |
| DNA Sequence | | |

CACTGGGCATTCCTGGCACCATGGATGACGCTGCTGTCCTCAAGCGACGAGGCTACCTCCTGGGGATAAAT
TTAGGAGAG
GGCTCCTATGCAAAGTAAAATCTGCTTACTCTGAGCGCCTGAGTTCAATGTGGCGATCAAGATCATCGA
CCGCAAGAAGGCCCCCGCAGACTTCTTGGAGAATTCCTTCCCCGGCAATTGAGATTCTGGCCATGTTAA
ACCACTGCTCCATCATTAAGACCTACGAGATCTTTGAGACATCACATGGCAAGGTCTACATCGTCATGGAG
CTCGCGGTCCAGGGCGACCTCCTCGAGTTAATCAACCCGGGGAGCCCTGCATGAGGACGAAGCTCGCAA
GAAGTTCCACCAGCTTTCCTTGGCCATCAAGTACTGCCACGACCTGGACGTCGTCCACCGGGACCTCAAGT
GTGACAACCTTCTCCTTGACAAGGACTTCAACATCAAGCTGTCCGACTTCAGCTTCTCCAAGCGCTGCCTG
CGGGATGACAGTGGTCGAATGGCATTAAAAGCAAGACCTTCTGTGGGTCACCAGCGTATGCGGCCCCAGAGGT
GCTGCAGGCCATTCCCTACCAGCCCAAGGTGTACGACATCTGGAGCCTAGGCGTGATCCTCTACATCATGG
TCTCCGGCTCCATGCCCTACGACGACTCCAACATCAAGAAAGATGCTGCGTATCCAGAAGGAGCACCGCGTC
AACTTCCCACGCTCCAAGCACCTGACAGGCGAGTGCAAGGACCTCATCTACCACATGCTGCAGCCCGACGT
CAACCGGCGGCTCCACATCGACGAGATCCTCAGCCACTGCTGGATGCAGCCCAAGGCACGCGGATCTCCCT
CTGTGGCCATCACAAGGAGGGGGAGAGTTCCCGGGGAACTGAACCCTTGTGGACCCCCGAACCTGGCTCT
GACAAGAAGTCTGCCACCAAGCTGGAGCCTGAGGGAGAGGCACAGCCCCAGGCACAGACTGAGGCAAAACC
CGAGGGGACAGCAATGCAAATGTCCAGGCACTCGGAGATCCTGGCTTTCCCCAGCAAGCCGTCGACTATGG
AGACAGAGGAAGGGCCCCCCCAACAGCCTCCAGAGACGCGCGCCCAGTGAGCTTCTTGCGGCCCAG

| | | | |
|---|---|---|---|
| NOV18h, 13376389 SNP for | SEQ ID NO: | 367 aa | SNP: Thr to Ala at position 326 |
| CG53270-01 | 294 | | |
| Protein Sequence | | | |

MDDAAVLKRRGYLLGINLGEGSYAKVKSAYSERLKFNVAIKIIDRKKAPADFLEKFLPREIEILAMLNHCS
IIKTYEIFETSHGKVYIVMELAVQGDLLELIKTRGALHEDEARKKFHQLSLAIKYCHDLDVThRDLKCDNL
LLDKDFNIKLSDFSFSKRCLRDDSGRMALSKTFCGSPAYAAPEVLQGIPYQPKVYDIWSLGVILYIMVCGS
MPYDDSMIKKMLRIQKEHRVNFPRSKHLTGECKDLIYHMLQPDVNRRLHIDEILSHCWMQPKARGSPSVAI
NKEGESSRGTEPLWTPEPGSDKKSATKLEPEGEAQPQAQPEAKPEGTANQMSRQSEILGFPSKPSTMETEE
GPPQQPPETRAQ

A ClustalW comparison of the above protein sequences yields the following sequence alignment shown in Table 18B.

TABLE 18B
Comparison of the NOV18 protein sequences.

| | |
|---|---|
| NOV18a | MDDAAVLKRRGYLLGINLGEGSYAKVKSAYSERLKFNVAIKIIDRKKAPADFLEKFLPRE |
| NOV18b | --------TGSYLLGINLGEGSYAKVKSAYSERLKFNVAIKIIDRKKAPADFLEKFLPRE |
| NOV18c | MDDAAVLKRRGYLLGINLGEGSYAKVKSAYSERLKFNVAIKIIDRKKAPADFLEKFLPRE |
| NOV18a | IEILANLNHCSIIKTYEIFETSNGKVYIVMELAVQGDLLELIKTRGALHEDEARkKFHQL |
| NOV18b | IEILAMLNHCSIIKTYEIFETSHGKVYIVMELAVQGDLLELIKTRGALHEDEARKKFHQL |
| NOV18c | IEILAMLNHCSIIKTYEIFETSHGKVYTVMELAVQGDLLELIKTRGALHEDEARKKFHQL |
| NOV18a | SLAIKYCHDLDVVHRDLKCDNLLLDKDFNIKLSDFSFSKRCLRDDSGRMALSKTFCGSPA |
| NOV18b | SLAIKYCHDLDVThRDLKCDNLLLOKDFNIKLSDFSFSKRCLRDDSGRMALSKTFCGSPA |
| NOV18c | SLAIKYCHDLDVVHRDLKCDNLLLDKDFNIKLSDFSFSKRCLRDDSGRMAlSKTFCGSPA |
| NOV18a | YAPEVLQGIPYQPKVYDIWSLGVILYIMVCGSMPYDDSNIKKMLRIQKEHRVNFPRSKH |
| NOV18b | YAPEVLQGIPYQPKVYDIWSLGVILYIMVCGSMPYDDSNIKKMLRTQKEHRVNFPRSKH |
| NOV18c | YAPEVLQGIPYQPKVYDIWSLGVILYIMVCGSMPYDDSNIKKMLRIQKEHRVNFPRSKH |
| NOV18a | LTGECKDLIYHMLQPDVNRRLHIDEILSNCWMQPKARGSPSVAINKEGESSRGTEPLWTP |
| NOV18b | LTGECKDLIYHMLQPDVNRRLHIDEILSHCWMGTG------------------------- |
| NOV18c | LTGECKDLIYHMLQPDVNRRLHIDEILSHCWMQPKARGSPSVAINKEGESSRGTEPLWTP |
| NOV18a | EPGSDKKSATKLEPEGEAQPQAQPETKPEGTAMQMSRQSEILGFPSKPSTMETEEGPPQQ |
| NOV18b | ------------------------------------------------------------ |
| NOV18c | EPOSDKKSATKLEPEGEAQPQAQPETKPEGTANQMSRQSEILGFPSKPSTMETEEGPPQQ |
| NOV18a | PFETPAQ |
| NOV18b | ------- |
| NOV18c | PPETRAQ |
| NOV18a | (SEQ ID NO: 280) |
| NOV18b | (SEQ ID NO: 282) |
| NOV18c | (SEQ ID NO: 284) |

Further analysis of the NOV18a protein yielded the following properties shown in Table 18C.

TABLE 18C
Protein Sequence Properties NOV18a

| SignalP | No Known Signal Sequence Predicted | | | |
|---|---|---|---|---|
| analysis: | | | | |
| PSORT II | PSG: a new signal peptide prediction method | | | |
| analysis: | N-region: length 10; pos. chg 3; neg. chg 2 | | | |
| | H-region: length 9; peak value 4.19 | | | |
| | PSG score: −0.21 | | | |
| | GvH: von Heijne's method for signal seq. recognition | | | |
| | GvH score (threshold: −2.1): −5.57 | | | |
| | possible cleavage site: between 24 and 25 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 1 | | | |
| | Number of TMS(s) for threshold 0.5: 0 | | | |
| | PERIPHERAL | Likelihood = | 1.11 (at 86) | |
| | ALOM score: −1.44 (number of TMSs: 0) | | | |
| | MITDISC: discrimination of mitochondrial targeting seq | | | |
| | R content: | 0 | Hyd Moment(75): | 8.48 |
| | Hyd Moment(95): | 7.98 | G content: | 0 |
| | D/E content: | 2 | S/T content: | 0 |

Score: −6.50
Gavel: prediction of cleavage sites for mitochondrial preseq
cleavage site motif not found
NUCDISC: discrimination of nuclear localization signals
pat4: none
pat7: none
bipartite: none
content of basic residues: 14.2%
NLS Score: −0.47
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: nuclear
Reliability: 76.7
COIL: Lupas's algorithm to detect coiled-coil regions
total: 0 residues
Final Results (k = 9/23):
60.9%: nuclear
17.4%: cytoplasmic
13.0%: peroxisomal
8.7%: mitochondrial
>> prediction for CG53270-01 is nuc (k = 9)

A search of the NOV18a protein against the Geneseq database, a proprietary database that contains sequences published in patents and patent publication, yielded several homologous proteins shown in Table 18D.

TABLE 18D
Geneseq Results for NOV18a

| | | NOV18a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAM47929 | Human htssk-1 SEQ ID NO 2 - | 1 . . . 367 | 367/367 (100%) | 0.0 |
| | Homo sapiens, 367 aa. | 1 . . . 367 | 367/367 (100%) | |
| | [WO200183768-A2, 08 NOV. | | | |
| | 2001] | | | |
| ABB05005 | Human kinase protein SEQ ID | 1 . . . 367 | 367/367 (100%) | 0.0 |
| | NO: 2 - Homo sapiens, 367 aa. | 1 . . . 367 | 367/367 (100%) | |
| | [WO200190328-A2, 29 NOV. | | | |
| | 2001] | | | |
| AAG78493 | Human 53070 protein kinase - | 1 . . . 367 | 367/367 (100%) | 0.0 |
| | Homo sapiens, 367 aa. | 1 . . . 367 | 367/367 (100%) | |
| | [WO200196544-A2, 20 DEC. 2001] | | | |
| ABP60983 | Novel human protein. SEQ ID 70 - | 1 . . . 367 | 367/367 (100%) | 0.0 |
| | Homo sapiens, 367 aa. | 1 . . . 367 | 367/367 (100%) | |
| | [WO200250105-A1, 27 JUN. 2002] | | | |
| ABG30415 | Human testis specific kinase | 1 . . . 367 | 367/367 (100%) | 0.0 |
| | (TSSK) 1 protein - Homo sapiens, | 1 . . . 367 | 367/367 (100%) | |
| | 367 aa. [WO200238732-A2, 16 | | | |
| | MAY 2002] | | | |

In a BLAST search of public sequence databases, the NOV18a protein was found to have homology to the proteins shown in the BLASTP data in Table 18E.

TABLE 18E
Public BLASTP Results for NOV18a

| | | NOV18a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9BXA7 | Serine/threonine kinase FKSG81 | 1 . . . 367 | 367/367 (100%) | 0.0 |
| | (Testis-specific serine/threonine | 1 . . . 367 | 367/367 (100%) | |
| | kinase 1) - Homo sapiens (Human), | | | |
| | 367 aa. | | | |
| Q61241 | Serine/threonine kinase - Mus | 1 . . . 364 | 307/364 (84%) | e−176 |
| | musculus (Mouse), 364 aa. | 1 . . . 362 | 328/364 (89%) | |
| Q8IY55 | Hypothetical protein - Homo sapiens | 1 . . . 352 | 241/353 (68%) | e−136 |
| | (Human), 358 aa. | 1 . . . 334 | 282/353 (79%) | |
| Q96PF2 | Testis specific serine/threonine | 1 . . . 352 | 241/353 (68%) | e−136 |
| | kinase 2 - Homo sapiens (Human), | 1 . . . 334 | 281/353 (79%) | |
| | 358 aa. | | | |
| O54863 | Protein kinase - Mus musculus | 1 . . . 340 | 246/347 (70%) | e−136 |
| | (Mouse), 357 aa. | 1 . . . 344 | 277/347 (78%) | |

PFam analysis predicts that the NOV18a protein contains the domains shown in the Table 18F.

TABLE 18F
Domain Analysis of NOV18a

| | | Identities/ | |
|---|---|---|---|
| | NOV18a | Similarities | |
| Pfam | Match | for the | Expect |
| Domain | Region | Matched Region | Value |
| pkinase | 12 . . . 272 | 94/304 (31%) | 5.5e−73 |
| | | 204/304 (67%) | |

### Example 19

The NOV19 clone was analyzed, and the nucleotide and encoded polypeptide sequences are shown in Table 19A.

TABLE 19A
NOV19 Sequence Analysis

| | | |
|---|---|---|
| NOV19a, CG54254-04 | SEQ ID NO: 295 | 2040 bp |
| DNA Sequence | ORF Start: ATG at 1 | ORE Stop: TGA at 2023 |

ATGGTGGTGGCACACCCCACCGCCACTGCCACCACCACGCCCACTGCCACTGTCACGGCCACCGTTGT
GATGACCACCGCCACCATGGACCTGCGGGACTGGCTGTTCCTCTGCTACGGGCTCATCGCCTTCCTGA
CGGAGGTCATCGACAGCACCACCTGCCCCTCGGTGTGCCGCTGCGACAACGGCTTCATCTACTGCAAC
GACCGGGGACTCACATCCATCCCCGCAGATATCCCTUATGACGCCACCACCCTCTATCTGCAGAACAA
CCAGATCAACAACGCTGGCATCCCCCACGACCTCAAGACCAAGGTCAACGTGCAGGTCATCTACCTAT
ACGAGAATGACCTGGATGAGTTCCCCATCAACCTGCCCCGCTCCCTCCGGGAGCTGCACCTGCAGG
AACAATGTGCGCACCATTGCCAGGGACTCGCTGGCCCGCATCCCGCTGCTGGAGAAGCTGCACCTGGA
TGACAACTCCGTGTCCACCGTCAGCATTGAGGAGGACGCCTTCGCCGACAGCAACAGCTCAAGCTGC
TCTTCCTGAGCCGGAACCACCTGAGCAGCATCCCCTCGGGGCTGCCGCACACGCTGGAGGAGCTGCGG
CTGGATGACAACCGCATCTCCACCATCCCGCTGCATGCCTTCAAGGGCCTCAACAGCCTGCGGCGCCT
GGTGCTGGACGGTAACCTGCTGGCCAACCAGCGCATCGCCGACGACACCTTCAGCCGCCTACAGAACC
TCACAGAGCTCTCGCTGGTGCGCAATTCGCTGGCCGCGCCACCCCTCAACCTGCCCAGCGCCCACCTG
CAGAACTCTACCTGCAGGACAATGCCATCAGCCACATCCCCTACAACACGCTGGCCAACATGCGTCA
GCTGGAGCGGCTGGACCTGTCCAACAACAACCTGACCACGCTGCCCCGCGGCCTGTTCGACGACCTGG
GGAACCTGGCCCAGCTGCTGCTCAGGAACAACCCTTGGTTTTGTGGCTGCAACCTCATGTGGCTGCGG
GACTGGGTGAAGGCACGGGCGGCCGTGGTCAACGTGCGGGGCCTCATGTGCCAGGGCCCTGAGAGGT
CCGGGGCATGGCCATCAAGGACATTACCAGCCAGATGGACGAGTGTTTTGAGACGGCGCCGCACCGCG
GCGTGGCCAATGCGGCTGCCAAGACCACGGCCAGCAACCACGCCTCTGCCACCACGCCCCAGGGTTCC
CTGTTTACCCTCAAGGCCAAAAGGCCAGGCCTGCGCCTCCCCGACTCCAACATTGACTACCCCATGGC
CACGGGTGATGGCCCCAAGACCCTGGCCATCCACGTGAAGGCCCTGACGGCAGACTCCATCCGCATCA
CGTGGAAGGCCACGCTCCCCGCCTCCTCTTTCCGGCTCAGTTGGCTGCGCCTGGGCCACAGCCCAGCC
GTGGGCTCCATCACGGAGACCTTGGTGCAGGGGGACAAGACAGAGTACCTGCTGACAGCCCTGGAGCC
CAAGTCCACCTACATCATCTGCATGGTCACCATGGAGACCAGCAATGCCTACGTAGCTGATGAGACAC
CCGTGTGTGCCAAGGCAGAGACAGCCGACAGCTATGGCCCTACCACCACACTCAACCAGGAGCAGAAC
GCTGGCCCCATGGCGAGCCTGCCCCTGGCGGGCATCATCGGCGGGGCAGTGGCTCTGGTCTTCCTCTT
CCTGGTCCTGCGGGCCATCTGCTGGTACGTGCACCAGGCTGGCGAGCTGCTGACCCGGGAGAGG
ACAACCGGGGCAGCAGGGAAAGGATGACTATATGGAGTCAGGGACCAAGAAGGATAACTCCATCCTG
GAATCCGCGCCCCTGGGCTGCAGATGCTGCCCATCAACCCGTACCGCGCCAAAGAGGAGTACGTGGT
CCACACTATCTTCCCCTCCAACGGCAGCAGCCTCTGCAAGGCCACACACACCATTGGCTATGGCACCA
CGCGGGGCTACCGGGACGGCGGCATCCCCGACATAGACTACTCCTACACATGATGCCCGCCCACCCGG

| | | | |
|---|---|---|---|
| NOV 19a, CG54254-04 | SEQ ID NO: 296 | 674 aa | MW at 74087.4 kD |
| Protein Sequence | | | |

MVVAHPTATATTTPTATVTATVVMTTATMDLRDWLFLCYGLIAFLTEVIDSTTCPSVCRCDNGFIYC
DRGLTSIPADIPDDATTLYLQNNQINNAGIPQDLKTKVNVQVIYLYENDLDEFPINLPRSLRELHLQD
NNVRTIARDSLARIPLLEKLHLDDNSVSTVSIEEDAFADSKQLKLLFLSLVRNHLSSIPSGLPHTLEELR
LDDNRISTIPLHAFKGLNSLRRLVLDGNLLANQRIADDTFSRLQNLTELSLVRNSLPPLNLPSAHL
QKLYLQDNAISHIPYNTLAKMRELERLDLSNNNLTTLPRGLFDDLGNLAQLLLRNNPWFCGCNLMWLR
DWVKARAAVVNVRGLMCQGPEKVRGMAIKDITSEMDECFETGPQGGVANAAAKTTASNHASATTPQGS
LFTLKAKPGLRLPDSNIDYPMATGDGAKTLAIHVKALTADSIRITWKATLPASSFRLSWLRLGHSPA
VGSITETLVQGDKTEYLLTALEPKSTYIICMVTMETSNAYVADETPVCAKAETADSYGPTTTLNQEQN
AGPMASLPLAGIIGGAVALVFLFLVLGAICWYVHQAGELLTRERAYNRGSREKDDYMESGTKKDNSIL
EIRGPGLQMLPINPYRAKEEYVVHTIFPSNGSSLCKATHTIGYGTTRGYRDGGIPDIDYSYT

| | | |
|---|---|---|
| NOV19b, 247846813 | SEQ ID NO: 297 | 1933 bp |
| DNA Sequence | ORF Start: at 2 | ORF Stop: end of sequence |

AGGCTCCGCGGCCGCCCCCTTCACCGGATCCATCGACAGCACCACCTGCCCCTCGGTGTGCCGCTGCG
ACAACGGCTTCATCTACTGCAACCACCGGGGACTCACATCCATCCCCGCAGATATCCCTGATGACGCC
CAACGTGCAGGTCATCTACCTATACGAGAATGACCTGGATGAGTTCCCCATCAACCTGCCCCGCTCCC
TCCGGGAGCTGCACCTGCAGGACAACAATGTGCGCACCATTGCCAGGGACTCGCTGGCCCGCATCCCG
CTGCTGGAGAAGCTGCACCTGGATGACAACTCCGTGTCCACCGTCAGCATTGAGGAGGACGCCTTCGC
CGACAGCAAACAGCTCAAGCTGCTCTTCCTGAGCCGGAACCACCTGAGCAGCATCCCCTCGGGGCTGC
CGCACACGCTGGAGGAGCTGCGGCTGGATCACAACCGCATCTCCACCATCCCGCTGCATGCCTTCAAG
GGCCTCAACAGCCTGCGGCGCCTGGTGCTGGACGGTAACCTGCTGGCCAACCAGCGCATCGCCGACGA
CACCTTCAGCCGCCTACAGAACCTCACAGACCTCTCGCTGGTGCGCAATTCGCTGGCCGCGCCACCCC
TCAACCTGCCCAGCGCCCACCTGCAGAAGCTCTACCTGCAGGACAATGCCATCAGCCACATCCCCTAC
AACACGCTGGCCAAGATGCGTGAGCTGGAGCGGCTGGACCTGTCCAACAACAACCTGACCACGCTGCC
CCGCGGCCTGTTCGACGACCTGGGGAACCTGGCCCAGCTGCTGCTCAGGAACAACCCTTGGTTTTGTG
GCTGCAACCTCATGTGGCTGCGGGACTGGGTGAAGGCACGGGCGGCCGTGGTCAACGTGCGGGGCCTC
ATGTGCCAGGGCCCTGAGAAGGTCCGGGGCATGGCCATCAAGGACATTACCAGCGAGATGGACGAGTG
TTTTGAGACGGGGCCGCAGGGCGCCGTGGCCAATGCGGCTGCCAAGACCACGGCCAGCAACCACGCCT
CTGCCACCACGCCCCAGGGTTCCCTGTTTACCCTCAAGGCCAAAAGGCCAGGGCTGCGCCTCCCCGAC
TCCAACATTGACTACCCCATGGCCACGGGTGATGGCGCCAAGACCCTGGCCATCCACGTGAAGGCCCT
GACGGCAGACTCCATCCGCATCACGTGGAAGGCCACGCTCCCCGCCTCCTCTTTCCGGCTCAGTTGGC
TGCGCCTGGGCCACAGCCCAGCCGTGGGCTCCATCACGGAGACCTTGGTGCAGGGGGACAAGACAGAG
TACCTGCTGACAGCCCTGGAGCCCAAGTCCACCTACATCATCTGCATGGTCACCATGGAGACCAGCAA
TGCCTACGTAGCTGATGAGACACCTGTGTGTGCCAAGGCAGAGACAGCCGACAGCTATGGCCCTACCA
CCACACTCAACCAGGAGCAGAACGCTGGCCCCATGGCGAGCCTGCCCCTGGCGGGCATCATCGGCGGG
GCAGTGGCTCTGGTCTTCCTCTTCCTGGTCCTGGGGGCCATCTGCTGGTACGTGCACCAGGCTGGCGA
GCTCCTGACCCGGGACAGGGCCTACAACCCGGGCAGCAGGAAAAGGATGACTATATGGAGTCAGGGA
CCAAGAAGGATAACTCCATCCTGGAAATCCGCGGCCCTGGGCTGCAGATGCTGCCCATCAACCCGTAC
CGCGCCAAAGAGGAGTACGTGGTCCACACTATCTTCCCCTCCAACGGCAGCAGCCTCTGCAAGGCCAC
ACACACCATTGGCTATGGCACCACGCGGGGCTACCGCGACGGCGGCATCCCCGACATAGACTACTCCT
ACACACTCGAGGGCAAGGGTGGGCGCGCC

| | | | |
|---|---|---|---|
| NOV19b, 247846813 | SEQ ID NO: 298 | 644 aa | MW at 70572.3 kD |
| Protein Sequence | | | |

GSAAAPFTGSIDSTTCPSVCRCDNGFIYCNDRGLTSIPADIPDDATTLYLQNNQINNAGIPQDLKTKV
NVQVIYLYENDLDEFPINLPRSLRELHLQDNNVRTIARDSLARIPLLEKLHLDDNSVSTVSIEEDAFA
DSKQLKLLFLSRNHLSSIPSGLPHTLEELRLDDNRISTIPLHAFKGLNSLRRLVLDGNLLANQRIADD
TFSRLQNLTELSLVRNSLAAPPLNLPSAHLQKLYLQDNAISHIPYNTLAKMRELERLDLSNNNLTTLP
RGLFDDLGNLAQLLLRNNPWFCGCNLMWLRDWVKARAAVNVRGLMCQGPEKVRGMAIKDITSEMDEC
FETGPQGGVANAAAKTTASNHASATTPQGSLFTLKAKRPGLRLPDSNIDYPMATGDGAKTLAIHVKAL
TADSIRITWKATLPASSFRLSWLRLGHSPAVGSITETLVQGDKTEYLLTALEPKSTYIICMVTMETSN
AYVADETPVCAKAETADSYGPTTTLNQEQNAGPMASLPLAGIIGGAVALVFLFLVLGAICWYVHQAGE
LLTRERAYNRGSRKKDDYMESGTKKDNSILEIRGPGLQMLPINPYRAKEEYVVHTIFPSNGSSLCKAT
HTIGYGTTRGYRDGGIPDIDYSYTLEGKGGRA

| | | |
|---|---|---|
| NOV19c, 247846825 | SEQ ID NO: 299 | 785 bp |
| DNA Sequence | ORF Start: at 2 | ORF Stop: 784 |

AGGCTCCGCGGCCGCCCCCTTCACCGGATCCGACGCCACCACCCTCTATCTGCAGAACAACCACATCA
GACCTGGATGAGTTCCCCATCAACCTGCCCCGCTCCCTCCGGGAGCTGCACCTGCAGGACAACAATGT
GCGCACCATTGCCACGGACTCGCTGCCCCGCATCCCGCTGCTGGAGAAGCTGCACCTGGATGACAACT
CCGTGTCCACCGTCAGCATTGAGGAGGACGCCTTCGCCGACAGCAACAGCTCAAGCTGCTCTTCCTG
AGCCGGAACCACCTGAGCAGCATCCCCTCGGGGCTGCCGCACACGCTGGAGGAGCTGCGGCTGGATGA
CAACCGCATCTCCACCATCCCGCTGCATGCCTTCAAGGGCCTCAACAGCCTGCGCCGCCTGGTGCTCG
ACGGTAACCTGCTGGCCAACCAGCGCATCGCCCACGACACCTTCAGCCGCCTACAGAACCTCACAGAG
CTCTCGCTGGTGCGCAATTCGCTGGCCGCGCCACCCCTCAACCTGCCCAGCGCCCACCTGCAGAACT
CTACCTGCAGGACAATGCCATCAGCCACATCCCCTACAACACGCTGGCCAAGATGCGTGAGCTGGAGC
GGCTGGACCTGTCCAACAACAACCTGACCACGCTGCCCCGCGGCCTGTTCGACGACCTGGGGCTCCAG
GGCAAGGGTGGGCGCGCCGACCCAGCTTTCTTGTACA

| | | | |
|---|---|---|---|
| NOV 19c, 247846825 | SEQ ID NO: 300 | 261 aa | MW at 29146.5 kD |
| Protein Sequence | | | |

GSAAAPFTGSDATTLYLQNNQINNAGIPQDLKTKVNVQVIYLYENDLDEFPINLPRSLRELHLQDNNV
RTTARDSLARIPLLEKLHLDDNSVSTVSIEEDAFADSKQLLFLSRNHLSSIPSGLPHTLEELRLDD
NRISTIPLHAFKGLNSLRRLVLDGNLLANQRIADDTFSRLQNLTELSLVRNSLPPLNLPSAHLQKL
YLQDNAISHIPYNTLAKMRELERLDLSNNNLTTLPRGLFDDLGLEGKGGRADPAFLY

| | | |
|---|---|---|
| NOV19d, 247846967 | SEQ ID NO: 301 | 1880 bp |
| DNA Sequence | | |
| | ORF Start: at 2 | ORE Stop: 1879 |

ACGCTCCGCGGCCGCCCCCTTCACCGGATCCGACGCCACCACCCTCTATCTGCAGAACAACCAGATCA
GACCTGGATGAGTTCCCCATCAACCTGCCCCGCTCCCTCCGGGAGCTGCACCTGCAGGACAACAATGT
GCGCACCATTGCCAGGGACTCGCTGGCCCGCATCCCGCTGCTGGAGAAGCTGCACCTGGATGACAACT
CCGTGTCCACCGTCAGCATTGAGGAGGACGCCTTCGCCGACAGCAACAGCTCAAGCTGCTCTTCCTG
AGCCGGAACCACCTGAGCAGCATCCCCTCGCGGCTGCCGCACACGCTGGAGGAGCTGCGGCTGGATGA
CAACCGCATCTCCACCATCCCGCTGCATGCCTTCAAGGGCCTCAACAGCCTGCGGCGCCTGGTGCTGG
ACGGTAACCTGCTGGCCAACCAGCGCATCGCCGACGACACCTTCAGCCGCCTACAGAACCTCACAGAG
CTCTCGCTGGTGCGCAATTCGCTGGCCGCGCCACCCCTCAACCTGCCCAGCGCCCACCTGCAGAAGCT
CTACCTGCAGGACAATGCCATCAGCCACATCCCCTACAACACGCTGGCCAAGATGCGTGAGCTGGAGC
GGCTGGACCTGTCCAACAACAACCTGACCACCCTGCCCCGCGGCCTGTTCGACGACCTGGGGAACCTG
GCCCAGCTGCTGCTCAGGAACAACCCTTGGTTTTGTGGCTGCAACCTCATGTGGCTGCGGGACTGGGT
GAGGCACGGGCGGCCGTGGTCAACGTGCGGGGCCTCATGTGCCAGGGCCCTGAGAAGGTCCGGGGCA
TGGCCATCAAGGACATTACCAGCGAGATGGACGAGTGTTTTGAGACGGGGCCGCAGGGCGGCGTCGCC
AATGCGGCTGCCAAGACCACGGCCAGCAACCACGCCTCTGCCACCACGCCCCAGGGTTCCCTGTTTAC
CCTCAACGCCAAAAGGCCAGGGCTCCGCCTCCCCGACTCCAACATTGACTACCCCATGGCCACGGGTG
ATGGCGCCAAGACCCTGGCCATCCACGTGAAGGCCCTGACGGCAGACTCCATCCGCATCACGTGGAAG
GCCACGCTCCCCGCCTCCTCTTTCCGGCTCAGTTGGCTGCGCCTGGGCCACAGCCCACCCGTGGGCTC
CATCACGGAGACCTTGGTGCAGGGGGACAAAGACAGAGTACCTGCTGACAGCCCTGGAGCCCAAGTCCA
CCTACATCATCTCCATGGTCACCATGGAGACCAGCAATGCCTACGTAGCTGATGAGACACCCGTGTGT
GCCAAGCAGAGACAGCCGACAGCTATGGCCCTACCACCACACTCAACCAGGAGCAGAACGCTGGCCC
CATGGCGAGCCTGCCCCTGGCGGGCATCATCGGCGGGGCAGTGGCTCTGGTCTTCCTCTTCCTGGTCC
TGGGGGCCATCTGCTGGTACGTGCACCAGGCTGGCGAGCTGCTGACCCGGGAGAGGGCCTACAACCGG
CGCCCCTGGGCTGCAGATGCTGCCCATCAACCCGTACCGCGCCAAGAGGAGTACGTGGTCCACACTA
TCTTCCCCTCCAACGGCAGCAGCCTCTGCAAGGCCACACACACCATTGGCTATGGCACCACGCGGGGC
TACCGGGACGGCGGCATCCCCGACATACACTACTCCTACACACTCGAGGGCAAGGGTGGGCGCCCCGA
CCCAGCTTTCTTGTACACAGCTGGCATTATAAGAAGCCATTGCT

| | | | |
|---|---|---|---|
| NOV19d, 247846967 | SEQ ID NO: 302 | 626 aa | MW at 68699.3 kD |
| Protein Sequence | | | |

GSAAAPFTGSDATTLYLQNNQINNAGIPQDLKTKVNVQVIYLYENDLDEFPINLPRSLRELHLQDNNV
YLQDNAISnIPYNTLAKMRELERLDLSNNNLTTLPRGLFDDLGNLAQLLLRNNPWFCGCNLMWLRDWV
KARAAVVNVRGLMCQGPEKVRGMAIKDITSEMDECFETGPQGGVANAAAKTTASNHASATTPQGSLFT
LKAKRPGLRLPDSNIDYPMATGDGAKTLAIHVKALTADSIRITWKATLPASSFRLSWLRLGHSPAVOS
ITETLVQGDKTEYLLTALEPKSTYIICMVTMETSNAYVADETPVCAKAETADSYGPTTTLNQEQNAGP
MASLPLAGIIGGAVALVFLVIGAICWYVHQAGELLTRERAYNRGSRKKDDYMESGTKKNSILEIR
GPCLQMLPINPYRAKEEYVVHTIFPSNGSSLCKATHTIGYGTTRCYRDGGIPDIDYSYTLEGKGGRPD
PAFLYTAGIIRSHC

| | | |
|---|---|---|
| NOV19e, 283841186 | SEQ ID NO: 303 | 2041 bp |
| DNA Sequence | ORF Start: at 2 | ORE Stop: end of sequence |

CACCGGATCCATGGTGGTGGCACACCCCACCGCCACTGCCACCACCACGCCCACTGCCACTGTCACGG
CCACCGTTGTCATGACCACGGCCACCATGGACCTGCGGGACTGGCTGTTCCTCTGCTACGCGCTCATC
GCCTTCCTGACGGAGGTCATCGACAGCACCACCTGCCCCTCGGTGTGCCGCTCCGACAACGGCTTCAT
ATCTACCTATACGAGAATGACCTGGATGAGTTCCCCATCAACCTGCCCCGCTCCCTCCGGGAGCTGCA
CCTGCAGGACAACAATGTGCGCACCATTGCCAGGGACTCGCTGGCCCGCATCCCGCTGCTGGAGAAGC
TGCACCTGGATGACAACTCCGTGTCCACCGTCAGCATTGAGGAGGACGCCTTCGCCGACAGCAACAG
CTCAAGCTGCTCTTCCTGAGCCGGAACCACCTGAGCAGCATCCCCTCGGGGCTGCCGCACACGCTGGA
GGAGCTGCGGCTGGATGACAACCGCATCTCCACCATCCCGCTGCATGCCTTCAAGGGCCTCAACAGCC
TGCGGCGCCTGGTGCTGGACGGTAACCTGCTGGCCAACCAGCGCATCGCCGACGACACCTTCAGCCGC
CTACAGAACCTCACAGAGCTCTCGCTGGTGCGCAATTCGCTGGCCGCGCCACCCCTCAACCTGCCCAG
CGCCCACCTGCAGAAGCTCTACCTGCAGGACAATGCCATCAGCCACATCCCCTACAACACGCTGGCCA
AGATGCGTGAGCTGGAGCGGCTGGACCTGTCCAACAACAACCTGACCACGCTGCCCCGCGGCCTGTTC
GACGACCTGGGGAACCTGGCCCAGCTGCTGCTCAGGAACAACCCTTGGTTTTGTGGCTGCAACCTCAT
CTGGCTGCGGGACTGGGTGAAGGCACGGGCGGCCGTGGTCAACGTGCGGGGCCTCATGTGCCAGGGCC
CTGAGAAGGTCCGGGGCATGGCCATCAAGGACATTACCAGCGAGATGGACGAGTGTTTTGAGACGGGG
CCGCAGGGCGGCGTGGCCAATGCGGCTGCCAACACCACGGCCAGCAACCACGCCTCTGCCACCACGCC
CCAGGGTTCCCTGTTTACCCTCAAGGCCAAAAGGCCAGGGCTGCGCCTCCCCGACTCCAACATTGACT
ACCCCATGGCCACGGGTGATGGCGCCAAGACCCTGGCCATCCACGTGAACCCCCTGACGGCAGACTCC
ATCCGCATCACGTGGAAGGCCACGCTCCCCGCCTCCTCTTTCCGGCTCAGTTGGCTGCCCCTGGGCCA
CAGCCCAGCCGTGGGCTCCATCACGGAGACCTTGGTGCAGGGGGACAAGACAGAGTACCTGCTGACAG
CCCTGGAGCCCAAGTCCACCTACATCATCTGCATGGTCACCATGGAGACCAGCAATGCCTACGTAGCT
GATGAGACACCCGTGTGTGCCAAGGCAGAGACAGCCGACAGCTATGGCCCTACCACCACACTCAACCA
GGAGCAGAACGCTGGCCCCATGGCGAGCCTGCCCCTGGCGGGCATCATCGGCGGGGCAGTGGCTCTGG
TCTTCCTCTTCCTGGTCCTGGGGGCCATCTGCTGGTACGTGCACCAGGCTGGCGAGCTGCTGACCCGG
GAGAGCCCCTACAACCGGGGCAGCAGGAAAAAGGATGACTATATGGAGTCAGGGACCAAGAAGGATAA
CTCCATCCTGGAAATCCGCCGCCCTGGGCTGCAGATGCTGCCCATCAACCCGTACCGCGCCAAAGAGG
AGTACGTGGTCCACACTATCTTCCCCTCCAACGGCAGCAGCCTCTGCAAGGCCACACACACCATTGGC
TATGGCACCACGCGGcIGCTACCGGGACGGCGGCATCCCCGACATAGACTACTCCTACACACTCGAGGG
C

| | | | |
|---|---|---|---|
| NOV19e, 283841186 | SEQ ID NO: 304 | 680 aa | MW at 74631.1 kD |
| Protein Sequence | | | |

TGSMVVAHPTATATTTPTATVTATVVMTTATMDLRDWLFLCYGLIAFLTEVIDSTTCPSVCRCDNGFI
YCNDRGLTSIPAlMPDDATTLYLQNNQINNAGIPQDLKTKVNVQVIYLYENDLDEFPINLPRSLRELH
LQDNNVRTIARDSLARIPLLEKLHLDDNSVSTVSIEEDAFADSKQLKLLFLSRIJHLSSIPSGLPHTLE
ELRLDDNRISTIPLHAFKGLNSLRRLVLDGNLLANQRIADDTFSRLQNLTELSLVRNSLAAPPLNLPS
AHLQKLYLQDNAISHIPYNTLAKMRELERLDLSNNNLTTLPRGLFDDLGNLAQLLLRNNPWFCGCNLM
WLRDWVKARAAVVNVRGLMCQGPEKVRGMAIKDITSEMDECFETGPQGGVANAAAKTTASNHASATTP
QGSLFTLKAKRPGLRLPDSNIDYPMATGDGAKTLAIHVKALTADSIRITWKATLPASSFRLSWLRLGH
SPAVGSITETLVQGDKTEYLLTALEPKSTYIICMVTMETSNAYVADETPVCAKAETADSYGPTTTLNQ
EQNAGPMASLPLAGIIGGAVALVFLFLVLCAILCWYVHQAGELLTRERAYNRGSRKKDDYMESGTKKDN
SILEIRGPGLQMLPINPYRAKEEYVVHTIFPSNGSSLCKATIGYGTTRGYRDGGIPDIDYSYTLEG

| | | |
|---|---|---|
| NOV19f, CG54254-01 | SEQ ID NO: 305 | 2025 bp |
| DNA Sequence | ORF Start: ATG at 1 | ORF Stop: TGA at 2023 |

ATGGTGGTCCCACACCCCACCGCCACTGCCACCACCACGCCCACTGCCACTGTCACGGCCACCGTTGT
GATGACCACGGCCACCATGGACCTGCGGGACTGGCTGTTCCTCTGCTACGGGCTCATCGCCTTCCTGA
CGGAGGTCATCGACAGCACCACCTGCCCCTCGGTGTGCCGCTGCGACAACGGCTTCATCTACTGCAAC
GACCGGGGACTCACATCCATCCCCGCAGATATCCCTGATGATGCCACCACCCTCTACCTGCAGAACAA
CCACATCAACAACGCCGGCATCCCCCAGGACCTCAAGACCAAGGTCAACGTGCAGGTCATCTACCTAT
ACGAGAATCACCTGGATGAGTTCCCCATCAACCTGCCCCGCTCCCTCCGGGAGCTGCACCTGCAGGAC
AACAATGTGCGCACCATTGCCAGGGACTCGCTGGCCCGCATCCCGCTGCTGGAGAAGCTCCACCTGGA
TGACAACTCCGTGTCCACCGTCAGCATTGAGGAGGACGCCTTCGCCGACAGCAAACACCTCAAGCTGC
TCTTCCTGAGCCGGAACCACCTGAGCAGCATCCCCTCGGGGCTGCCGCACACGCTGGAGGAGCTGCGG
CTGGATGACAACCGCATCTCCACCATCCCGCTGCATGCCTTCAAGGGCCTCAACAGCCTGCGGCGCCT
GGTGCTGGACGGTAACCTGCTGGCCAACCAGCGCATCGCCGACGACACCTTCAGCCGCCTACAGAACC
TCACAGAGCTCTCGCTGGTGCGCAATTCGCTGGCCGCGCCACCCCTCAACCTGCCCAGCGCCCACCTG
CAGAAGCTCTACCTGCAGGACAATGCCATCAGCCACATCCCCTACAACACGCTGGCCAAGATGCGTGA
GCTGGAGCGGCTGGACCTGTCCAACAACAACCTGACCACGCTGCCCCGCGGCCTGTTCGACGACCTGG
GGAACCTGGCCCACCTCCTUCTCAGGAACAACCCTTGGTTTTGTGGCTGCAACCTCATGTGGCTGCGG
GACTGGGTGAAGGCACGGGCGGCCGTGGTCAACGTGCGGGGCCTCATGTGCCAGGGCCCTGAGAAGGT
CCCGGCCATGCCCATCAAGGACATTACCAGCGAGATGGACGAGTGTTTTGAGACGGGGCCGCAGGGCG
GCGTGGCCAATGCGGCTGCCAAGACCACGGCCAGCAACCACGCCTCTGCCACCACGCCCCAGGGTTCC
CTGTTTACCCTCAAGGCCAAAAGGCCAGGGCTGCGCCTCCCCGACTCCAACATTGACTACCCCATGGC
CACGGGTGATGGCGCCAAGACCCTGGCCATCCACGTGAAGGCCCTGACGGCAGACTCCATCCGCATCA
CGTGGAAGGCCACGCTCCCCGCCTCCTCTTTCCGGCTCAGTTGGCTGCGCCTGGGCCACAGCCCAGCC
GTGGGCTCCATCACGGAGACCTTGGTGCAGGGGGACAAGACAGAGTACCTCCTGACAGCCCTGGAGCC
CAAGTCCACCTACATCATCTGCATGGTCACCATGGAGACCAGCAATGCCTACCTAGCTGATGAGACAC
CCGTGTGTGCCAAGGCAGAGACAGCCGACAGCTATGGCCCTACCACCACACTCAACCAGGAGCAGAAC
GCTGGCCCCATGGCGAGCCTGCCCCTCCCGGCCATCATCGGCGGGGCAGTGGCTCTGGTCTTCCTCTT
CCTGGTCCTCCGGGGCCATCTGCTGGTACGTGCACCAGGCTGGCGAGCTGCTGACCCGGGAGAGGGCCT
ACAACCGGGGCAGCAGGAAAAAGGATGACTATATGGAGTCAGGGACCAAGAAGGATAACTCCATCCTG
GAAATCCGCCCCCCTGGGCTGCAGATGCTCCCCATCAACCCGTACCGCGCCAAAGAGGAGTACGTGGT
CCACACTATCTTCCCCTCCAACGGCAGCAGCCTCTGCAAGGCCACACACACCATTGGCTACGGCACCA
CGCGGGGCTACCGGGACGGCGGCATCCCCGACATAGACTACTCCTACACATGA

| | | | |
|---|---|---|---|
| NOV 19f, CG54254-01 | SEQ ID NO: 306 | 674 aa | 1MW at 74086.5 kD |
| Protein Sequence | | | |

MVVAHPTATATTTPTATVTATVVMTTATMDLRDWLFLCYGLIAFLTEVIDSTTCPSVCRCDNGFIYCN
DRGLTSIPADIPDDATTLYLQNNQINNAGILPQDLKTKVNVQVIYLYENDLDEPPINLPRSLRELHLQD
NNVRTIARDSLARIFLLEKLHLDDNSVSTVSIEEDAFADSKQLKLLFLSRNHLSSIPSGLPHTLEELR
LDDNRTSTIPLHAFKGLNSLRRLVLDGNLLANQRIADDTFSRLQNLTELSLVRNSLAAPPLNLPSANL
QKLYLQDNAISNIPYNTLAKMRELERLDLSNNNLTTLPRGLFDDLGNLAQLLLRNNPWFCGCNLMWLR
DWVKARAAVVNVRGLMCQGPEKVRGMAIKDITSEMDECFETGPQGGVANAAAKTTASNHASATTPQGS
LFTLKAKRPGLRLPDSNIDYPMATGDGAKTLAIHVKALTADSIRITWKATLPASSFRLSWLRLGHSPA
VGSTTETLVQGDKTEYLLTALEPKSTYIICMVTMETSNAYVADETPVCAKAETADSYGPTTTLNQEQN
AGPMASLPLAGIIGGAVALVFLFLVLGAICWYVHQAGELLTRENAYNRGSRKKDDYMESGTKKDNSIL
EIRGPGLQMLPINPYRAKEEYVVHTIFPSNGSSLCKATHTIGYGTTRGYRDGGIPDIDYSYT

| | | |
|---|---|---|
| NOV19g, CG54254-02 | SEQ ID NO: 307 | 1995 bp |
| DNA Sequence | ORF Start: ATG at | jORF Stop: TGA at 1993 |

ATGGTGGTGGCACACCCCACCGCCACTGCCACCACCACGCCCACTGCCACTGTCACGGCCACCGTTGT
GATGACCACGGCCACCATGGACCTGCGGGACTGGCTGTTCCTCTGCTACGGGCTCATCGCCTTCCTGA
CGGAGGTCATCGACAGCACCACCTCCCCCTCGGTGTGCCGCTGCGACAACGGCTTCATCTACTGCAAC
GACCGGGGACTCACATCCATCCCCGCAGATATCCCTGATGATGCCACCACCCTCTACCTGCACAACAA
CCAGATCAACAACGCCGGCATCCCCCAGGACCTCAAGACCAAGGTCAACGTGCAGGTCATCTACCTAT
ACGAGAATGACCTGGATGAGTTCCCCATCAACCTGCCCCGCTCCCTCCGGGAGCTGCACCTGCAGGAC
AACAATGTGCGCACCATTGCCAGGGACTCGCTGGCCCGCATCCCGCTGCTGGAGAAGCTGCACCTGGA
TGACAACTCCGTGTCCACCGTCAGCATTGAGGAGGACGCCTTCGCCGACAGCAAACAGCTCAAGCTGC
TCTTCCTGAGCCGGAACCACCTGAGCAGCATCCCCTCGGGGCTGCCGCACACGCTGGAGGAGCTGCGG
CTGGATGACAACCGCATCTCCACCATCCCGCTGCATGCCTTCAAGGCCCTCAACAGCCTGCGGCGCCT
GGTGCTGGACGGTAACCTGCTGGCCAACCAGCGCATCGCCGACGACACCTTCAGCCGCCTACAGAACC
TCACAGAGCTCTCGCTGGTGCGCAATTCGCTGGCCGCGCCACCCCTCTACCTGCAGGACAATGCCATC
AGCCACATCCCCTACAACACGCTGGCCAAGATGCGTGAGCTGGAGCGGCTGGACCTGTCCAACAACAA
CCTGACCACGCTGCCCCGCGGCCTGTTCGACGACCTGGGGAACCTGGCCCAGCTGCTGCTCAGGAACA
ACCCTTGGTTTTGTGGCTGCAACCTCATGTGGCTGCGGGACTGGGTGAAGGCACGGGCGGCCGTGGTC
AACGTGCGGGGCCTCATGTGCCAGGGCCCTGAGAAGGTCCGGGGCATGGCCATCAAGGACATTACCAG
CGAGGTGGAGAGTGTTTTGAGACGGGCGCCGCAGGGCGGCGTGGCCAATGCGGCTGCCAAGACCACGG
CCAGCAACCACGCCTCTGCCACCACGCCCCAGGGTTCCCTGTTTACCCTCAAGGCCAAAAGGCCAGGG
CTGCGCCTCCCCGACTCCAACATTGACTACCCCATGGCCACGGGTGATGGCGCCAAGACCCTGGCCAT
CCACGTGAAGGCCCTGACGGCAGACTCCATCCGCATCACGTGGAAGGCCACGCTCCCCGCCTCCTCTT
TCCGGCTCAGTTGGCTCCGCCTGGGCCACAGCCCAGCCGTGGGCTCCATCACGGAGACCTTGGTGCAG
GGGGACAAGACAGACTACCTGCTGACAGCCCTGGAGCCCAAGTCCACCTACATCATCTGCATGGTCAC
CATGGAGACCAGCAATGCCTACGTAGCTGATGAGACACCCGTGTGTGCCAAGGCAGAGACAGCCGACA
GCTATGGCCCTACCACCACACTCAACCAGGAGCAGAACGCTGGCCCCATGGCGAGCCTGCCCCTGGCG
CGCATCATCGGCGGGGCAGTGGCTCTGGTCTTCCTCTTCCTGGTCCTGGGGGCCATCTGCTCCTACGT
GCACCAGGCTGGCGAGCTGCTGACCCGGGAGACGGCCTACAACCGGGGCAGCAGGAAAAAGGATGACT
ATATGGAGTCAGGGACCAAGAACGATAACTCCATCCTGGAAATCCGCGGCCCTGCCCTGCAGATGCTG
CCCATCAACCCGTACCGCGCCAAACAAGAGTACGTGGTCCACACTATCTTCCCCTCCAACGGCAGCAG
CCTCTGCAAGGCCACACACACCATTGGCTACGGCACCACGCGGGGCTACCGGGACGGCGGCATCCCCG
ACATAGACTACTCCTACACATGA

| | | | |
|---|---|---|---|
| NOV19g, CG54254-02 | SEQ ID NO: 308 | 664 aa | 1MW at 72982.3 kD |
| Protein Sequence | | | |

MVVAHPTATATTTPTATVTATVVMTTATMDLRDWLFLCYGLTAFLTEVIDSTTCPSVCRCDNGFIYCN
DRGLTSIPADIPDDATTLYLQNNQINNAGIPQDLKTKVNVQVIYLYENDLDEFPINLPRSLRELHLQD
NNVRTIARDSLARIPLLEKLHLDDNSVSTVSIEEDAFADSKQLKLLFLSRNHLSSIPSGLPHTLEELR
LDDNRISTIPLHAFKGLNSLRRLVLDGNLLANQRIADDTFSRLQNLTELSLVRNSLAAPPLYLQDNAI
SLITPYNTLAKMRELERLDLSNNNLTTLPRGLFDDLGNLAQLLLRNNPWFCGCNLMWLRDWVKARAAVV
NVRGLMCQCPEKVRGMAIKDITSEVESVLRRAPQGGVANAAAKTTASNHASATTPQGSLFTLKAKRPG
LRLPDSNIDYPMATGDGAKTLAIHVKALTADSIRITWKATLPASSFRLSWLRLGHSPAVGSITETLVQ
GDKTEYLLTALEFKSTYIICMVTMETSNAYVADETPVCAKAETADSYGPTTTLNQEQNAGPMASLPLA
GIIGGAVALVFLFLVLGAICWYVHQAGELLTRERAYNRGSRKKDDYMESGTKKDNSILEIRGPGLQML
PINPYRAKEEYVVHTIFPSNGSSLCKATHTIGYGTTRGYRDGGIPDIDYSYT

| | | |
|---|---|---|
| NOV19h, CG54254-03 | SEQ ID NO: 309 | 1485 bp |
| DNA Sequence | ORF Start: at 1 | ORF Stop: end of sequence |

ACCACCTGCCCCTCGGTGTGCCGCTGCGACAACGGCTTCATCTACTGCAACGACCGGGGACTCACATC
CATCCCCGCAGATATCCCTGATGATGCCACCACCCTCTACCTGCAGAACAACCAGATCAACAACCCCG
GCATCCCCCAGGACCTCAAGACCAAGGTCAACGTGCAGGTCATCTACCTATACGAGAATGACCTGGAT
GAGTTCCCCATCAACCTGCCCCGCTCCCTCCGGGAGCTGCACCTGCAGGACAACAATGTGCGCACCAT
TGCCAGGGACTCGCTGGCCCGCATCCCGCTGCTGGAGAAGCTGCACCTGGATGACAACTCCGTGTCCA
CCGTCAGCATTGAGGAGGACGCCTTCGCCGACAGCAAACAGCTCAAGCTGCTCTTCCTGAGCCGGAAC
CACCTGAGCAGCATCCCCTCGGGGCTGCCCCACACGCTGGAGGAGCTGCGGCTGGATGACAACCGCAT
CTCCACCATCCCGCTGCATGCCTTCAAGGGCCTCAACAGCCTGCGGCGCCTGGTGCTGGACGGTAACC
TGCTGGCCAACCAGCGCATCGCCGACGACACCTTCAGCCGCCTACACAACCTCACAGAGCTCTCGCTG
GTGCGCAATTCGCTGGCCGCGCCACCCCTCAACCTGCCCAGCGCCCACCTGCAGAAGCTCTACCTGCA
GGACAATGCCATCAGCCACATCCCCTACAACACGCTGGCCAAGATGCGTGAGCTGGAGCGGCTGGACC
TGTCCAACAACAACCTGACCACGCTGCCCCGCGGCCTGTTCGACGACCTGGGGAACCTGGCCCAGCTG
CTCCTCAGGAACAACCCTTGGTTTTGTGGCTGCAACCTCATGTGGCTGCGGGACTGGGTGCCGCACG
GGCGGCCCTGGTCAACGTGCGGGGCCTCATGTGCCAGGGCCCTGAGAAGGTCCGGGGCATGGCCATCA
AGGACATTACCAGCGAGATGGACGAGTGTTTTGAGACGGGGCCGCAGGGCGGCGTGGCCAATGCGGCT
GCCAAGACCACGGCCAGCAACCACCCCTCTGCCACCACGCCCCAGGGTTCCCTGTTTACCCTCAAGGC
CAAAAGGCCAGGGCTGCGCCTCCCCCACTCCAACATTGACTACCCCATGGCCACGGGTGATGGCGCCA
AGACCCTGGCCATCCACGTGAACGCCCTGACGGCAGACTCCATCCGCATCACGTGGAAGGCCACGCTC
CCCGCCTCCTCTTTCCGGCTCAGTTGGCTCCGCCTGGGCCACAGCCCAGCCGTGGGCTCCATCACGGA
GACCTTGGTGCAGGGGGACAAGACACAGTACCTGCTGACAGCCCTGGAGCCCAAGTCCACCTACATCA
TCTGCATGGTCACCATGGAGACCAGCAATGCCTACGTAGCTCATGAGACACCCGTGTGTGCCAAGGCA
GAGACAGCCGACAGCTATGGCCCTACCACCACACTCAACCAGGAGCAGAACGCTGGC

| | | | |
|---|---|---|---|
| NOV19h, CG54254-03 | SEQ ID NO: 310 | 495 aa | MW at 54572.3 kD |
| Protein Sequence | | | |

TTCPSVCRCDNGFIYCNDRGLTSIPADIPDDATTLYLQNNQINNAGIPQDLKTKVNVQVIYLYENDLD
EFPINLPRSLRELHLQDNNVRTIARDSLARIPLLEKLHLDDNSVSTVSIEEDAFADSKQLKLLFLSRN
HLSSIPSGLPHTLEELRLDDNRISTIPLIHAFKGLNSLRRLVLDGNLLANQRIADDTFSRLQNLTELSL
VRNSLAAPPLNLPSAHLQKLYLQDNAISHIPYNTLAKMRELERLDLSNNNLTTLPRGLFDDLGNLAQL
LLRNNPWFCGCNLMWLRDWVKARAAVVNVRGLMCQGPEKVRGMAIKDITSEMDECFETGPQGGVANAA
AKTTASNHASATTPQGSLFTLKAKRPGLRLPDSNIDYPMATGDGAKTLAIHVKALTADSIRITWKATL
PASSFRLSWLRLGHSPAVGSITETLVQGDKTEYLLTALEPKSTYIICMVTMETSNAYVADETPVCAKA
ETADSYGPTTTLNQEQNAG

| | | |
|---|---|---|
| NOV19i, CG54254-05 | SEQ ID NO: 311 | 2041 bp |
| DNA Sequence | ORF Start: ATG at 11 | ORF Stop: end of sequence |

CACCGGATCCATGGTGGTGGCACACCCCACCGCCACTGCCACCACCACCCCCACTGCCACTGTCACGG
CCACCGTTGTGATCACCACGGCCACCATGGACCTGCGGGACTGGCTGTTCCTCTGCTACGGGCTCATC
GCCTTCCTGACGGAGGTCATCGACAGCACCACCTGCCCCTCGGTGTGCCGCTGCGACAACGGCTTCAT
CTACTGCAACGACCGGGGACTCACATCCATCCCCGCAGATATCCCTGATGACGCCACCACCCTCTACC
TGCAGAACAACCAGATCAACAACGCTGGCATCCCCCAGGACCTCAAGACCAAGGTCAACGTGCAGGTC
ATCTACCTATACGAGAATGACCTGGATGAGTTCCCCATCAACCTGCCCCGCTCCCTCCGGGAGCTGCA
CCTGCAGGACAACAATGTGCGCACCATTGCCAGGGACTCGCTGCCCCGCATCCCGCTGCTGGAGAAGC
TGCACCTGGATGACAACTCCGTGTCCACCGTCAGCATTGAGGAGGACGCCTTCGCCGACAGCAAACAG
CTCAAGCTGCTCTTCCTGAGCCGGAACCACCTGAGCAGCATCCCCTCGGGGCTGCCGCACACGCTGGA
GGAGCTGCCCGCTGGATGACAACCGCATCTCCACCATCCCGCTGCATGCCTTCAAGGGCCTCAACAGCC
TGCCCCCCCTGGTCCTGGACGGTAACCTGCTGGCCAACCAGCGCATCGCCGACGACACCTTCAGCCGC
CTACAGAACCTCACAGAGCTCTCGCTGGTGCGCAATTCGCTGGCCGCGCCACCCCTCAACCTGCCCAG
CGCCCACCTGCAGAAGCTTTACCTGCAGGACAATGCCATCAGCCACATCCCCTACAACACGCTGGCCA
AGATGCGTGAGCTGGAGCGGCTGGACCTGTCCAACAACAACCTGACCACGCTGCCCCGCGGCCTGTTC
GACGACCTGGGGAACCTGGCCCAGCTGCTGCTCACGAACAACCCTTGGTTTTGTGGCTGCAACCTCAT
GTGGCTGCGGGACTGGGTGAAGGCACGGGCGGCCGTGGTCAACGTGCGGGGCCTCATGTGCCAGGGCC
CTGAGAAGGTCCGGGGCATGGCCATCAAGGACATTACCAGCCAGATGGACGAGTGTTTTGAGACGGGG
CCGCAGGGCGGCGTGGCCAATGCGGCTGCCAAGACCACGGCCAGCAACCACGCCTCTGCCACCACGCC
CCAGGGTTCCCTGTTTACCCTCAAGGCCAAAAGGCCAGGGCTGCGCCTCCCCGACTCCAACATTGACT
ACCCCATGGCCACGGGTGATGGCGCCAAGACCCTGGCCATCCACGTGAAGGCCCTGACGGCAGACTCC
ATCCGCATCACGTGGAAGGCCACGCTCCCCGCCTCCTCTTTCCGGCTCAGTTGGCTGCGCCTGGGCCA
CAGCCCAGCCGTGGGCTCCATCACGGAGACCTTGGTGCAGGGGGACAAGACAGAGTACCTGCTGACAG
CCCTGGAGCCCAAGTCCACCTACATCATCTGCATGGTCACCATGGAGACCAGCAATGCCTACGTAGCT
GATGAGACACCCGTGTGTGCCAAGCCAGAGACAGCCGACAGCTATGGCCCTACCACCACACTCCCCCA
GGAGCAGAACGCTGGCCCCATGGCGAGCCTGCCCCTGGCGGGCATCATCGGCGGGGCAGTGGCTCTGG
TCTTCCTCTTCCTGGTCCTGGGGGCCATCTGCTGGTACGTGCACCAGGCTGGCGAGCTGCTGACCCGG
GAGAGGGCCTACAACCGGGGCAGCAGGAAAAGGATGACTATATGGAGTCACGGACCAAGAAAGATAA
CTCCATCCTGGAATCCGCCCCCCTGCGCTGCAGATGCTGCCCATCAACCCGTACCGCCCCGAGG
AGTACGTGGTCCACACTATCTTCCCCTCCAACGGCAGCAGCCTCTGCAAGGCCACACACACCATTGGC
TATGGCACCACGCGGGGCTACCGGGACGGCGGCATCCCCGACATAGACTACTCCTACACA
C

| | | | |
|---|---|---|---|
| NOV19i, CG54254-05 | SEQ ID NO: 312 | 674 aa | MW at 74086.5 kD |
| Protein Sequence | | | |

MVVAHIPTATATTTPTATVTATVVMTTATMDLRDWLFLCYGLIAFLTEVIDSTTCPSVCRCDNGFIYCN
DRGLTSIPADIPDDATTLYLQNNQINNAGIPQDLKTKVNVQVIYLYENDLDEFPINLPRSLRELHLQD
NNVRTIARDSLARIPLLEKLHLDDNSVSTVSIEEDAFADSKQLKLLFLSRNHLSSIPSGLPHTLEELR
LDDNRISTIPLHAFKGLNSLRRLVLDGNLLANQRIADDTFSRLQNLTELSLVRNSLAAPPLNLPSAHL
QKLYLQDNAISIPYNTLAKMRELERLDLSNNNLTTLPRGLFDDLGNLAQLLLRNNPWFCGCNLMWLR
DWVKARAAVVNVRGLMCQGPEKVRGMAIKDITSEMDECFETGPQGGVANAAAKTTASNHASATTPQGS
LFTLKAKRPGLRLPDSNIDYPMATGDGAKTLAIHVKALTADSIRITWKATLPASSFRLSWLRLGHSPA
VGSITETLVQGDKTEYLLTALEPKSTYIICMVTMETSNAYVADETPVCAKAETADSYGPTTTLNQEQN
AGPMASLPLAGIIGGAVALVFLFLVLGAICWYVIHQAGELLTRERAYNRGSRKKDDYMESGTKKDNSIL
EIRGPGLQMLPINPYRAKEEYVVHTIFPSNGSSLCKATHTIGYGTTRGYRDGGIPDIDYST

| | | |
|---|---|---|
| NOV19j, CG54254-06 | SEQ ID NO: 313 | 2039 bp |
| DNA Sequence | ORF Start: at 1 | ORF Stop: TAG at 2020 |

ACCGCCACTGCCACCACTACGCCCACTGCCACTGTCACGGCCACCGTTGTGATGACCACGCCCACCAT
GGACCTGCGGGACTGGCTGTTCCTCTGCTACGGGCTCATCGCCTTCCTGACGGAGGTCATCGACACCA
CCACCTGCCCCTCGGTGTGCCGCTGCGACAACGGCTTCATCTACTGCAACGACCGGGGACTCACATCC
ATCCCCGCAGATATCCCTGATGACGCCACCACCCTCTACCTGCAGAACAACCAGATCAACAACGCCGG
CATCCCCCAGGACCTCAAGACCAAGGTCAACGTGCAGGTCATCTACCTATACGAGAATGACCTGGATG
AGTTCCCCATCAACCTGCCCCGCCCCCTCCGGGAGCTGCACCTGCAGGACAACAATGTGCGCACCATT
GCCAGGGACTCGCTGGCCCGCATCCCGCTGCTGGAGAAGCTGCACCTGCATGACAACTCCGTGTCCAC
CGTCAGCATTGAGGAGGACGCCTTCGCCGACAGCAAACAGCTCAAGCTGCTCTTCCTGAcACCGGAACC
ACCTGAGCAGCATCCCCTCGGGGCTGCCGCACACGCTGGAGGAGCTGCGGCTGGATGACAACCGCATC
TCCACCATCCCGCTGCATGCCTTCAAGGGCCTCAACAGCCTGCGGCGCCTGGTGCTGGACGGTAACCT
GCTGGCCAACCAGCGCATCGCCGACGACACCTTCAGCCGCCTACAGAACCTCACAGAGCTCTCGCTGG
TGCGCAATTCGCTGGCCGCGCCACCCCTCAACCTGCCCAGCGCCCACCTGCAGAAGCTCTACCTGCAG
GACAATGCCATCAGCCACATCCCCTACAACACGCTGGCCAAGATGCGTGAGCTGGAGCGGCTGGACCT
GTCCAACAACAACCTGACCACGCTGCCCCGCGGCCTGTTCGACGACCTGGGGAACCTGGCCCAGCTGC
TGCTCAGGAACAACCCTTGGTTTTGTGGCTGCAACCTCATGTGGCTGCGGGACTGGGTGAAGGCACGG
GCGGCCGTGGTCAACGTGCGGGGCCTCATGTGCCAGGGCCCTGAGAAGGTCCGGGGCATGGCCATCAA
GGACATTACCAGCGAGATGGACGAGTGTTTTGAGACGGGGCCGCAGGGCGGCGTGGCCAATGCGGCTG
CCAAGACCACGGCCAGCAACCACGCCACTGCCACCACGCCCCAGGGTTCCCTGTTTACCCTCAAGGCC
AAAACGCCAGGGCTGCGCCTCCCCCACTCCAACATTGACTACCCCATGGCCACGGGTGATGGCGCCAA
GACCCTGCCCATCCACGTCAAGGCCCTGACGGCAGACTCCATCCGCATCACGTGGAAGGCCACGCTCC
CCGCCTCCTCTTTCCGGCTCAGTTGGCTGCGCCTGGGCCACAGCCCAGCCGTGGGCTCCATCACGGAC
ACCTTGGTGCAGGGGGACAAGACAGAGTACCTGCTGACAGCCCTGGAGCCCAAGTCCACCTACATCAT
CTGCATGGTCACCATGGAGACCAGCAATGCCTATGTAGCTGATGAGACACCCGTGTGTGCCAAGGCAG
AGACAGCCGACAGCTATGGCCCTACCACCACACTCAACCAGGAGCAGAACGCTGGCCCCATGGCGAGC
CTGCCCCTGGCGGGCATCATCGGCGGGGCAGTGGCTCTGGTCTTCCTCTTCCTGGTCCTGGGGGCCAT
CTGCTGGTACGTGCACCAGGCTGGCGAGCTGCTGACCCGGGAGAGGGCCTACAACCGGGGCAGCAGGA
AAAAGGATGACTATATGGAGTCAGGGACCAAGAAGGATAACTCCATCCTGGAAATCCGCGGCCCTGGG
CTCCAGATGCTGCCCATCAACCCGTACCGCGCCAAAGAGGAGTACGTGGTCCACACTATCTTCCCCTC
CAACGGCAGCAGCCTCTGCAAGGCCACACACACCATTGGCTACGGCACCACGCGGGGCTACCGCCACT
GCCACCACCTCCCCGACATAGACTACTCCTACACACGATCCCCGACATAGACTACTCCTACACATGA

| | | | |
|---|---|---|---|
| NOV19j, CG54254-06 | SEQ ID NO: 314 | 673 aa | MW at 74202.6 kD |
| Protein Sequence | | | |

TATATTTPTATVTATVVMTTATMDLRDWLFLCYCLIAFLTEVIDSTTCPSVCRCDNGFIYCNDRGLTS
IPADIPDDATTLYLQNNQINNAGIPQDLKTKVNVQVIYLYENDLDEFPINLPRPLRELHLQDNNVRTI
ARDSLARIPLLEKLHLDDNSVSTVSIEEDAFADSKQLKLLFLSRNHLSSIPSGLPHTLEELRLDDNRI
STIPLHAFKGLNSLRRLVLDGNLLANQRIADDTFSRLQNLTELSLVRNSLAAPPLNLPSAHLQKLYLQ
DNAISHIPYNTLAKNRELERLDLSNNNLTTLPRGLFDDLGNLAQLLLRNNPWFCCCNLMWLRDWVKAR
AAVVNVRGLMCQCPEKVRGMAIKDITSEMDECFETGPQGGVANAAAKTTASNHATATTPQGSLFTLKA
KRPGLRLPDSNIDYPMATGDGAKTLAIHVKALTADSIRITWKATLPASSFRLSWLRLGHSPAVGSITE
TLVQGDKTEYLLTALEPKSTYIICMVTMETSNAYVADETPVCAKAETADSYGPTTTLNQEQNAGPMAS
LPLAGIIGGAVALVFLFLVLGAICWYVHQAGELLTRERAYNRGSRKKDDYMESGTKKDNSILEIRCPG
LQMLPINPYRAKEEYVVHTIFPSNGSSLCKATHTIGYGTTRGYRHCHHLPDIDYSYTRSPT

| | | |
|---|---|---|
| NOV19k, CG54254-07 | SEQ ID NO: 315 | 2049 bp |
| DNA Sequence | ORF Start: ATG at 16 | ORF Stop: TAG at 2038 |

CACCGCGGCCGCACCATGGTGGTGGCACACCCCACCGCCACTGCCACCACCACGCCCACTGCCACTGT
CACGGCCACCGTTGTGATGACCACGGCCACCATGGACCTGCGGGACTGGCTGTTCCTCTGCTACGGGC
TCATCCCCTTCCTGACGGAGGTCATCGACAGCACCACCTGCCCCTCGGTGTGCCGCTGCGACAACGGC
TTCATCTACTGCAACGACCGGGGACTCACATCCATCCCCGCACATATCCCTGATGACGCCACCACCCT
CTACCTGCAGAACAACCAGATCAACAACGCTGGCATCCCCCACGACCTCAACACCAAGGTCAACGTGC
AGGTCATCTACCTATACGAGAATGACCTGGATGAGTTCCCCATCAACCTGCCCCGCTCCCTCCGGGAG
CTGCACCTGCAGGACAACAATGTGCGCACCATTGCCAGGGACTCGCTGGCCCGCATCCCGCTGCTGGA
GAAGCTGCACCTGGATGACAACTCCGTGTCCACCGTCAGCATTGAGGAGGACGCCTTCGCCGACACCA
AACAGCTCAAGCTGCTCTTCCTGAGCCGGAACCACCTGAGCAGCATCCCCTCGGGGCTGCCCCACACG
CTGGAGGAGCTGCGGCTGGATGACAACCGCATCTCCACCATCCCGCTGCATGCCTTCAAGGGCCTCAA
CAGCCTGCGGCGCCTCGTGCTGGACGGTAACCTGCTGGCCAACCAGCGCATCGCCGACGACACCTTCA
GCCGCCTACAGAACCTCACAGAGCTCTCGCTGGTGCGCAATTCCCTGGCCGCGCCACCCCTCAACCTG
CCCAGCGCCCACCTGCAGAAGCTCTACCTGCAGGACAATGCCATCAGCCACATCCCCTACAACACGCT
GGCCAAGATGCGTGAGCTGGAGCGGCTGGACCTGTCCAACAACAACCTGACCACGCTGCCCCGCGGCC
TGTTCGACGACCTGGGGAACCTGGCCCAGCTGCTGCTCAGGAACAACCCTTGGTTTTGTGGCTGCAAC
CTCATGTGGCTGCGGGACTGGGTGAAGGCACGGGCGGCCGTGGTCAACGTGCGGGGCCTCATGTGCCA
GGGCCCTGAGAAGGTCCGGGGCATGGCCATCAAGGACATTACCAGCGAGATGGACGAGTGTTTTCAGA
CGGGGCCGCAGGGCGGCGTGGCCAATGCGGCTGCCAAGACCACGGCCAGCAACCACGCCTCTCCCACC
ACGCCCCAGGGTTCCCTGTTTACCCTCAAGGCCAAAAGGCCAGGGCTGCGCCTCCCCGACTCCAACAT
TGACTACCCCATGCCCACGGGTGATGGCGCCAAGACCCTGGCCATCCACCTGAAGGCCCTGACGGCAG
ACTCCATCCGCATCACGTGGAAGGCCACGCTCCCCGCCTCCTCTTTCCGGCTCAGTTGGCTGCGCCTG
GGCCAATCCCAGCCGTGGGCTCCATCACGGAGACCTTGGTGCAGGGGGACAAGACAGAGTACCTGCT
GACAGCCCTGGAGCCCAAGTCCACCTACATCATCTGCATGGTCACCATGGAGACCAGCAATGCCTACG
TAGCTGATGAGACACCCGTGTGTGCCAAGGCAGAGACAGCCGACAGCTATGGCCCTACCACCACACTC
AACCAGGAGCAGAACGCTGGCCCCATGGCGAGCCTGCCCCTGGCGGGCATCATCGGCGGGGCAGTGGC
TCTCGTCTTCCTCTTCCTGGTCCTGGGGGCCATCTGCTCGTACGTGCACCAGGCTGGCGAGCTGCTGA
CCCGGAGAGGGCCTACAACCGGGGCAGCAGGAAAAAGGATGACTATATGGAGTCAGGGACCAAGAAG
GATAACTCCATCCTGGAAATCCGCGGCCCTGGGCTCCAGATGCTGCCCATCAACCCGTACCGCGCCAA
AGAGGAGTACGTGGTCCACACTATCTTCCCCTCCAACGGCAGCAGCCTCTGCAAGGCCACACACACCA
TTGGCTATGGCACCACGCGGGGCTACCGGGACGGCGGCATCCCCGACATAGACTACTCCTACACATAG
GTCGACGGC

| | | | |
|---|---|---|---|
| NOV19k, CG54254-07 | SEQ ID NO: 316 | 674 aa | MW at 74086.5 kD |
| Protein Sequence | | | |

MVVAHPTATATTTPTATVTATVVMTTATMDLRDWLFLCYGLIAFLTEVIDSTTCPSVCRCDNGFIYCN
DRGLTSIPADIPDDATTLYLQNNQINNAGIPQDLKTKVNVQVIYLYENDLDEFPINLPRSLRELHLQD
NNVRTIARDSLARIPLLEKLIHLDDNSVSTVSIEEDAFADSKQLKLLFLSRNHLSSIPSGLPHTLEELR
LDDNRISTIPLHAFKGLNSLRRLVLDGNLLANQRIADDTFSRLQNLTELSLVRNSLAAPPLNLPSAHIL
QKLYLQDNAISIIPYNTLAKMRELERLDLSNNNLTTLPRGLFDDLGNLAQLLLRNNPWFCGCNLMWLR
DWVKARAAVVNVRGLMCQGPEKVRGMAIKDITSEMDECFETGPQGGVANAAAKTTASNHASATTPQGS
LFTLKAKRPGLRLPDSNIDYPMATGDGAKTLAIHVKALTADSIRITWKATLPASSFRLSWLRLGHSPA
VGSITETLVQGDKTEYLLTALEPKSTYIICMVTMETSNAYVADETPVCAKAETADSYGPTTTLNQEQN
AGPMASLPLAGIIGGAVALVFLFLVLGAICWYVHQAGELLTRERAYNRGSRKKDDYMESGTKKDNSIL
EIRGPGLQMLPINPYRAKEEYVVHTIFPSNGSSLCKATHTIGYGTTRGYRDGGIPDIDYSYT

| | | |
|---|---|---|
| NOV19l, 13375078 SNP for | SEQ ID NO: | 2040 bp SNP: 34 A/G |
| CG54254-04 | 317 | |
| DNA Sequence | ORF Start: | ORF Stop: IGA at 2023 |
| | ATG at 1 | |

ATGGTGGTGGCACACCCCACCGCCACTGCCACCGCCACCCCCACTGCCACTGTCACGGCCACCGTTGTGA
TGACCACGCCCACCATGGACCTGCGGGACTGGCTGTTCCTCTGCTACGGGCTCATCGCCTTCCTGACGGA
GGTCATCGACAGCACCACCTGCCCCTCGGTGTGCCGCTGCGACAACGGCTTCATCTACTGCAACGACCGG
GGACTCACATCCATCCCCGCAGATATCCCTGATGACGCCACCACCCTCTATCTGCAGAACAACCAGATCA
ACAACGCTGGCATCCCCCAGGACCTCAAGACCAAGGTCAACGTGCAGGTCATCTACCTATACGAGAATGA
CCTGGATGAGTTCCCCATCAACCTGCCCCGCTCCCTCCGGGAGCTGCACCTGCAGGACAACAATGTGCGC
ACCATTGCCAGGGACTCGCTGGCCCGCATCCCGCTGCTGGAGAAGCTGCACCTGGATGACAACTCCGTCT
CCACCCTCAGCATTGAGGAGGACGCCTTCGCCGACACCAAACAGCTCAAGCTGCTCTTCCTGAGCCGGAA
CCACCTGAGCAGCATCCCCTCGGGGCTGCCGCACACGCTGGAGGAGCTGCAGCTGAAATGACAACCGCATC
TCCACCATCCCGCTGCATGCCTTCAAGGGCCTCAACAGCCTGCGGCGCCTGGTGCTGGACGGTAACCTGC
TGCCCAACCAGCGCATCGCCGACGACACCTTCAGCCGCCTACAGAACCTCACAGAGCTCTCGCTGGTGCG
CAATTCGCTGGCCGCGCCACCCCTCAACCTGCCCAGCGCCCACCTGCAGAAACTCTACCTGCAGTGCAAT
GCCATCAGCCACATCCCCTACAACACGCTGGCCAAGATGCGTGAGCTGGAGCGGCTCGACCTGTCCAACA
ACAACCTGACCACGCTGCCCCGCGGCCTGTTCGACGACCTGGGGAACCTGCCCCAGCTGCTGCTCAGGAA
CAACCCTTGGTTTTGTGGCTGCAACCTCATGTGGCTGCGGGACTGGGTGAAGGCACGGGCGGCCGTGGTC
AACGTGCGGGGCCTCATGTGCCAGGGCCCTGAGAAGGTCCGGGGCATGGCCATCAAGGACATTACCAGCG
AGATGGACGAGTGTTTTCAGACGGGGCCGCAGGGCGGCGTGGCCAATGCGGCTGCCAAGACCACGGCCAG
CAACCACGCCTCTGCCACCACGCCCCAGGGTTCCCTGTTTACCCTCAAGGCCAAAAGGCCAGGGCTGCGC
CTCCCCGACTCCAACATTGACTACCCCATGGCCACGGGTGATGCCGCCAAGACCCTGGCCATCCACGTGA
AGGCCCTGACGGCAGACTCCATCCGCATCACGTGGAACGCCACGCTCCCCGCCTCCTCTTTCCGGCTCAG
TTGGCTCCGCCTGGGCCACAGCCCAGCCGTGGGCTCCATCACGGAGACCTTGGTGCAGGGGGACAAGACA
CAGTACCTGCTGACAGCCCTGGAGCCCAAGTCCACCTACATCATCTGCATGGTCACCATGGAGACCAGCA
ATGCCTACGTAGCTGATGAGACACCCCTGTGTGCCAAGGCAGAGACAGCCGACAGCTATGGCCCTACCAC
CACACTCAACCAGGAGCAGAACGCTGGCCCCATGGCGAGCCTGCCCCTGGCGGGCATCATCGGCGGGGCA
GTGGCTCTGGTCTTCCTCTTCCTGGTCCTGGGGGCCATCTGCTGGTACGTGCACCAGGCTGGCGAGCTGC
TGACCCGGGAGAGGGCCTACAACCGGGGCAGCAGGGAAAAGGATGACTATATGGAGTCAGGGACCAAGAA
GGATAACTCCATCCTCGAAATCCGCGGCCCTGGGCTGCAGATGCTGCCCATCAACCCGTACCGCGCCAAA
GAGGAGTACGTCCTCCACACTATCTTCCCCTCCAACGGCACCACCCTCTGCAAGGCCACACACACCATTG
GCTATGGCACCACGCGGGGCTACCGGGACGGCGCCATCCCCGACATAGACTACTCCTACACATGATGCCC
GCCCACCCGG

| | | | |
|---|---|---|---|
| NOV 19l, 13375078 SNP for | SEQ ID NO: | 674 aa | SNP: Thr to Ala at position 12 |
| CG54254-04 | 318 | | |
| Protein Sequence | | | |

MVVAHPTATATATPTATVTATVVMTTATMDLRDWLFLCYGLIAFLTEVIDSTTCPSVCRCDNGFIYCNDR
GLTSTPADIPDDATTLYLQNNQINNAGIPQDLKTKVNVQVIYLYENDLDEFPINLPRSLRELHLQDNNVR
TIARDSLARIPLLEKLHLDDNSVSTVSIEEDAFADSKQLKLLFLSRNHLSSIPSGLPHTLEELRLDDNRI
STIPLHAFKGLNSLRRLVLDGNLLANQRIADDTFSRLQNLTELSLVRNSLAAPPLNLPSAHLQKLYLQDN
AISHIPYNTLAKMRELERLDLSNNNLTTLPRGLFDDLGNLAQLLLRNNPWFCGCMLMWLRDWVKARAAVV
NVRCLMCQGPEKVRGMAIKDITSEMDECFETCPQGGVANAAAKTTASNHASATTPQGSLFTLKAKRPGLR
LPDSNIDYPMATGDGAKTLAIHVKALTADSIRITWKATLPASSFRLSWLRLGHSPAVGSITETLVQGDKT
EYLLTALEPKSTYIICMVTMETSNAYVADETPVCAKAETADSYGPTTTLNQEQNAGPMASLPLAGIIGGA
VALVFLFLVLGAICWYVHQAGELLTRERAYNRGSREKDDYMESGTKKDNSILEIRGPGLQMLPINPYRAK
EEYVVHTIFPSNGSSLCKATHTIGYGTTRGYRDGGIPDIDYSYT

| | | |
|---|---|---|
| NOV19m, 13376406 SNP for | SEQ ID NO: 319 | 2040 bp SNP: 47 C/T |
| CG5425404 | ORF Start: ATG at 1 | ORF Stop: TGA at 2023 |
| DNA Sequence | | |

ATGGTGGTGGCACACCCCACCGCCACTGCCACCACCACGCCCACTGTCACTGTCACGGCCACCGTTGTGAT
GACCACGGCCACCATGGACCTGCGGGACTGGCTGTTCCTCTGCTACGGGCTCATCGCCTTCCTGACGGAGG
TCATCGACAGCACCACCTGCCCCTCGGTGTGCCGCTGCGACAACGGCTTCATCTACTGCAACGACCGGGGA
CTCACATCCATCCCCGCAGATATCCCTGATGACGCCACCACCCTCTATCTGCAGAACAACCAGATCAACAA
CGCTGGCATCCCCCAGGACCTCAAGACCAAGGTCAACGTGCAGGTCATCTACCTATACGAGAATGACCTGG
ATGAGTTCCCCATCAACCTGCCCCGCTCCCTCCGGGAGCTGCACCTGCAGGACAACAATGTGCGCACCATT
GCCAGGGACTCGCTGGCCCGCATCCCGCTGCTGGAGAAGCTGCACCTGGATGACAACTCCGTGTCCACCGT
CAGCATTGAGGAGGACGCCTTCGCCGACAGCAAACAGCTCAAGCTGCTCTTCCTGAGCCGGAA
CCACCTGAGCAGCATCCCCTCGGGGCTGCCGCACACGCTGGAGGAGCTGCGGCTGGATGACAACCGCATCT
CCACCATCCCGCTGCATGCCTTCAAGGGCCTCAACAGCCTGCGGCGCCTGGTGCTGGACGGTAACCTGCTG
GCCAACCAGCGCATCGCCGACGACACCTTCAGCCGCCTACAGAACCTCACAGAGCTCTCGCTGGTGCGCAA
TTCGCTGGCCGCGCCACCCCTCAACCTGCCCAGCGCCCACCTGCAGAAACTCTACCTGCAGGACAATGCCA
TCAGCCACATCCCCTACAACACGCTGGCCAAGATGCGTGAGCTGGAGCGGCTGGACCTGTCCAACAACAAC
CTGACCACGCTGCCCCGCGGCCTGTTCGACGACCTGGGGAACCTGGCCCAGCTGCTGCTCAGGAACAACCC
TTGGTTTTGTGGCTGCAACCTCATGTGGCTGCGGGACTGGGTGAAGGCACGGGCGGCCGTGGTCAACGTGC
GGGGCCTCATGTGCCAGGGCCCTGAGAAGGTCCGGGGCATGGCCATCAAGGACATTACCAGCGAGATGGAC
GAGTGTTTTGAGACGGGGCCGCAGGGCGGCGTGGCCAATGCGGCTGCCAAGACCACGGCCAGCAACCACGC
CTCTGCCACCACGCCCCAGGGTTCCCTGTTTACCCTCAAGGCCAAAAGGCCAGGGCTGCGCCTCCCCGACT
CCAACATTGACTACCCCATGGCCACGGGTGATGGCGCCAAGACCCTGGCCATCCACGTGAAGGCCCTGACG
GCAGACTCCATCCGCATCACGTGGAAGGCCACGCTCCCCGCCTCCTCTTTCCGGCTCAGTTGGCTGCGCCT
GGGCCACAGCCCAGCCGTGGGCTCCATCACGGAGACCTTGGTGCAGGGGGACAAGACAGAGTACCTGCTGA
CAGCCCTGGAGCCCAAGTCCACCTACATCATCTGCATGGTCACCATGGAGACCAGCAATGCCTACGTAGCT
GATGAGACACCCGTGTGTGCCAAGGCAGAGACAGCCGACAGCTATGGCCCTACCACCACACTCAACCAGGA
GCAGAACGCTGGCCCCATGGCGAGCCTGCCCCTGGCGGGCATCATCGGCGGGGCAGTGGCTCTGGTCTTCC
TCTTCCTGGTCCTGGGGGCCATCTGCTGGTACGTGCACCAGGCTGGCGAGCTGCTGACCCGGGAGAGGGCC
TACAACCGGGGCAGCAGGGAAAAGGATGACTATATGGAGTCAGGGACCAAGAAGGATAACTCCATCCTGGA
AATCCGCGGCCCTCGGCTGCAGATGCTGCCCATCAACCCGTACCGCGCCAAAGAGGAGTACGTGGTCCACA
CTATCTTCCCCTCCAACGGCAGCAGCCTCTGCAAGGCCACACACACCATTGGCTATGGCACCACGCGGGGC
TACCGGGACGGCGGCATCCCCGACATAGACTACTCCTACACATGATGCCCCCCCACCCGG

| | | | |
|---|---|---|---|
| NOV19m, 13376406 SNP for | SEQ ID NO: | 674 aa | SNP: Ala to Val at position 16 |
| CG54254-04 | 320 | | |
| Protein Sequence | | | |

MVVAHPTATATTTPTVTVTATVVMTTATMDLRDWLFLCYGLIAFLTEVIDSTTCPSVCRCDNGFIYCNDRG
LTSIPADIPDDATTLYLQNNQINNAGIPQDLKTKVNVQVIYLYENDLDEFPINLPRSLRELHLQDNNVRTI
ARDSLARIPLLEKLHLDDNSVSTVSIEEDAFADSKQLKLLFLSRNHLSSIPSGLPHTLEELRLDDNRISTI
PLHAFKCLNSLRRLVLDGNLLANQRIADDTFSRLQNLTELSLVRNSLAAPPLNLPSAIILQKLYLQDNAISH
IPYNTLAKMRELERLDLSNNNLTTLPRGLFDDLGNLAQLLLRNNPWFCGCNLMWLRDWVKARAAVVNVRGL
MCQCPEKVRCMAIKDITSEMDECFETGPQGGVANAAAKTTASNHASATTPQGSLFTLKAKRPGLRLPDSNI
DYPMATGDGAKTLAIHVKALTADSIRILTWKATLPASSFRLSWLRLGHSPAVGSITETLVQGDKTEYLLTAL
EPKSTYIICMVTMETSNAYVADETPVCAKAETADSYGPTTTLNQEQNAGPMASLPLAGIIGGAVALVFLFL
VLGATCWYVHQAGELLTRERAYNRGSREKDDYMESGTKKDNSILEIRGPGLQMLPINPYRAKEEYVVHTIF
PSNGSSLCKATHTIGYGTTRGYRDGGIPDIDYSYT

| | | |
|---|---|---|
| NOV19n, 13375079 SNP for | SEQ ID NO: 321 | 2040 bp SNP: 106 T/C |
| CG5425404 | ORF Start: ATG at 1 | ORF Stop: TGA at 2023 |
| DNA Sequence | | |

ATGCTGGTGGCACACCCCACCGCCACTGCCACCACCACGCCCACTGCCACTGTCACGGCCACCGTTGTGAT
GACCACGGCCACCATGGACCTGCGGGACTGGCTGCTCCTCTGCTACGCCCTCATCGCCTTCCTGACGGAGG
TCATCGACAGCACCACCTGCCCCTCGGTGTGCCGCTGCGACAACGGCTTCATCTACTGCAACGACCGGGGA
CTCACATCCATCCCCGCAGATATCCCTGATGACGCCACCACCCTCTATCTGCAGAACAACCAGATCAACAA
CGCTGGCATCCCCCAGGACCTCAAGACCAAGGTCAACGTGCAGGTCATCTACCTATACGAGAATGACCTGG
ATCAGTTCCCCATCAACCTGCCCCGCTCCCTCCGGGAGCTGCACCTGCAGGACAACAATGTGCGCACCATT
GCCAGGGACTCGCTCGCCCGCATCCCGCTGCTGGAGAAGCTGCACCTGGATGACAACTCCGTGTCCACCGT
CAGCATTGAGGAGGACGCCTTCGCCGACAGCAAACAGCTCAAGCTGCTCTTCCTGAGCCGGAACCACCTGA
GCAGCATCCCCTCGGGGCTGCCGCACACGCTGGAGGAGCTGCGGCTGGATGACAACCGCATCTCCACCATC
CCGCTGCATGCCTTCAAGGGCCTCAACAGCCTGCCCCGCCTGGTGCTGGACGGTAACCTGCTGGCCAACCA
GCGCATCGCCGACGACACCTTCAGCCGCCTACAGAACCTCACAGAGCTCTCGCTGGTGCGCAATTCGCTGG
CCGCGCCACCCCTCAACCTGCCCAGCGCCCACCTGCAGAAACTCTACCTGCAGGACAATGCCATCAGCCAC
ATCCCCTACAACACGCTGGCCAAGATGCGTGAGCTGGAGCGGCTGGACCTGTCCAACAACAACCTGACCAC
GCTGCCCCGCGGCCTGTTCGACGACCTGGGGAACCTGGCCCAGCTGCTGCTCAGGAACAACCCTTGGTTTT
GTGGCTGCAACCTCATGTGGCTGCGGGACTGGGTGAAGGCACGGGCGGCCGTGGTCAACUTGCGGGGCCTC
ATGTGCCAGGGCCCTGAGAAGGTCCGGGGCATGGCCATCAAGGACATTACCAGCGAGATGGACGAGTGTTT
TGAGACGGGGCCGCAGGGCGGCGTGGCCAATGCGGCTGCCAAGACCACGGCCAGCAACCACGCCTCTGCCA
CCACGCCCCAGGGTTCCCTGTTTACCCTCAAGGCCAAAAGGCCAGGGCTGCGCCTCCCCGACTCCAACATT
GACTACCCCATGCCCACGGGTGATCCCGCCAAGACCCTGGCCATCCACGTGAAGGCCCTGACGGCAGACTC
CATCCGCATCACGTCCAAGGCCACGCTCCCCGCCTCCTCTTTCCGGCTCAGTTGGCTGCGCCTGGGCCACA
GCCCAGCCGTGGGCTCCATCACGGAGACCTTGGTCCAGGGCGACAAGACAGAGTACCTGCTGACAGCCCTG
GAGCCCAAGTCCACCTACATCATCTGCATGGTCACCATGGAGACCAGCAATGCCTACGTAGCTGATGAGAC
ACCCGTGTGTGCCAAGGCAGAGACAGCCGACAGCTATGGCCCTACCACCACACTCAACCAGGAGCAGAACG
CTGGCCCCATGGCGAGCCTGCCCCTGGCGGGCATCATCGGCGGGGCAGTGGCTCTGGTCTTCCTCTTCCTG
GTCCTGGGGGCCATCTGCTGGTACGTGCACCAGGCTGGCGAGCTGCTGACCCGGGAGAGCGCCTACAACCG
GGGCAGCAGGGAAAAGGATGACTATATGGAGTCAGGGACCAAGAAGGATAACTCCATCCTGGAAATCCGCC
GCCCTGGGCTGCAGATGCTGCCCATCAACCCGTACCGCGCCAAAGAGGAGTACGTGCTCCACACTATCTTC
CCCTCCAACGGCAGCAGCCTCTGCAAGGCCACACACACCATTGGCTATGGCACCACGCCGGGCTACCGGGA
CGGCGGCATCCCCGACATAGACTACTCCTACACATGATGCCCGCCCACCCGG

| | | | |
|---|---|---|---|
| NOV19n, 13375079 SNP for | SEQ ID NO: | 674 aa | SNP: Phe to Leu at position 36 |
| CG54254-04 | 322 | | |
| Protein Sequence | | | |

MVVAHPTATATTTPTATVTATVVMTTATMDLRDWLLLCYGLIAFLTEVIDSTTCPSVCRCDNGFIYCNDRG
LTSIPADIPDDATTLYLQNNQINNAGIPQDLKTKVNVQVIYLYENDLDEFPINLPRSLRELHLQDNNVRTI
ARDSLARIPLLEKLHLDDNSVSTVSIEEDAFADSKQLKLLFLSRNHLSSIPSGLPHTLEELRLDDNRISTI
PLHAFKGLNSLRRLVLDGNLLANQRIADDTFSRLQNLTELSLVRNSLAAPPLNLPSAHLQKLYLQDNAISB
IPYNTLAKMRELERLDLSNNNLTTLPRGLFDDLGNLAQLLLRNNPWFCGCNLMWLRDWVKARAAVXTNVRGL
MCQGPEKVRGMAIKDITSEMDECFETGPQGGVANAAAKTTASNHASATTPQGSLFTLKAKRPGLRLPDSNI
DYPMATGDGAKTLAIHIVKALTADSIRITWKATLPASSFRLSWLRLGHSPAVGSITETLVQGDKTEYLLTAL
EPKSTYIICMVTMETSNAYVADETPVCAKAETADSYGPTTTLNQEQNAGPMASLPLAGIIGGAVALVFLFL
VLGAICWYVHQAGELLTRERAYNRGSREKDDYMESGTKKDNSILEIRGPGLQMLPINPYRAKEEYVVHTIF
PSNGSSLCKATHTIGYGTTRGYRDGGIPDIDYSYT

| | | |
|---|---|---|
| NOV19o, 13376405 SNP for | SEQ ID NO: 323 | 2040 bp SNP: 344 A/T |
| CG5425404 | ORF Start: ATG at | IORF Stop: TGA at 2023 |
| DNA Sequence | | |

ATGGTGGTGGCACACCCCACCGCCACTGCCACCACCACGCCCACTCCCACTGTCACGGCCACCGTTGTGAT
GACCACGGCCACCATGGACCTGCGGGACTGGCTGTTCCTCTGCTACGGGCTCATCGCCTTCCTGACGGAGG
TCATCGACAGCACCACCTGCCCCTCGGTGTGCCGCTGCGACAACGGCTTCATCTACTGCAACGACCGGGGA
CTCACATCCATCCCCGCAGATATCCCTGATGACGCCACCACCCTCTATCTGCAGAACAACCAGATCAACAA
CGCTGGCATCCCCCAGGACCTCAACACCAAGGTCAACCTGCAGGTCATCTACCTATACGTGAATGACCTGG
ATGAGTTCCCCATCAACCTGCCCCGCTCCCTCCGGGAGCTGCACCTGCAGGACAACAATGTGCGCACCATT
GCCACGGACTCGCTCGCCCGCATCCCGCTGCTGGAGAAGCTGCACCTGGATGACAACTCCGTGTCCACCGT
CAGCATTCAGGAGGACGCCTTCGCCGACAGCAAACAGCTCAAGCTGCTCTTCCTGAGCCGGAACCACCTGA
GCAGCATCCCCTCGGGGCTGCCGCACACGCTGGAGGAGCTGCGGCTGGATGACAACCGCATCTCCACCATC
CCGCTGCATGCCTTCAAGGGCCTCAACAGCCTGCGGCGCCTGGTGCTGGACGGTAACCTGCTGGCCAACCA
GCGCATCGCCGACGACACCTTCAGCCGCCTACAGAACCTCACAGAGCTCTCGCTGGTGCGCAATTCGCTGG
CCGCGCCACCCCTCAACCTGCCCAGCGCCCACCTGCAGAAACTCTACCTGCAGGACAATGCCATCAGCCAC
ATCCCCTACAACACGCTGGCCAAGATGCGTGAGCTGGAGCGGCTGGACCTGTCCAACAACAACCTGACCAC
GCTGCCCCGCGGCCTGTTCGACGACCTGGGGAACCTGGCCCAGCTGCTGCTCAGGAACAACCCTTCGTTTT
GTGGCTGCAACCTCATGTGGCTGCGGCACTGCGTCAAGGCACCGGCGGCCGTGGTCAACGTGCGGGGCCTC
ATGTGCCAGGGCCCTGAGAAGCTCCCCGCCATGGCCATCAAGGACATTACCAGCGAGATGGACGAGTGTTT
TCAGACGCCGCCGCAGGGCCCCGTGGCCAATGCGGCTGCCAAGACCACGGCCAGCAACCACGCCTCTGCCA
CCACCCCCCAGGGTTCCCTGTTTACCCTCAAGGCCAAAAGGCCAGGGCTGCGCCTCCCCGACTCCAACATT
GACTACCCCATGGCCACGGGTGATGGCGCCAAGACCCTGGCCATCCACGTGAAGGCCCTGACGGCAGACTC
CATCCGCATCACGTGGAAGGCCACGCTCCCCGCCTCCTCTTTCCGGCTCAGTTGGCTGCGCCTGGGCCACA
GCCCAGCCGTGGGCTCCATCACGGAGACCTTGGTGCAGGGGGACAAGACAGAGTACCTGCTGACAGCCCTG
GAGCCCAAGTCCACCTACATCATCTGCATGGTCACCATGGAGACCAGCAATGCCTACGTAGCTGATGAGAC
ACCCGTGTGTGCCAAGGCAGAGACAGCCGACAGCTATGGCCCTACCACCACACTCAACCAGGAGCAGAACG
CTGGCCCCATGGCGAGCCTGCCCCTGGCGGGCATCATCGGCGGGGCAGTGGCTCTGGTCTTCCTCTTCCTG
GTCCTGGGGGCCATCTGCTGGTACGTGCACCAGGCTGGCGAGCTGCTGACCCGGGACAGGGCCTACAACCG
GGGCAGCAGGGAAAAGGATGACTATATGGAGTCACGGACCAAGAAGCATAACTCCATCCTGGAAATCCCCG
GCCCTGGGCTGCAGATGCTGCCCATCAACCCCTACCGCGCCAAAGAGGAGTACGTGGTCCACACTATCTTC
CCCTCCAACGGCAGCAGCCTCTGCAACGCCACACACACCATTGGCTATGGCACCACGCGGGGCTACCGGGA
CGGCGGCATCCCCGACATAGACTACTCCTACACATGATGCCCGCCCACCCGG

| | | | |
|---|---|---|---|
| NOV19o, 13376405 SNP for | SEQ ID NO: | 674 aa | Glu to Val at position 115 |
| CG54254-04 | 324 | | |
| Protein Sequence | | | |

MVVAHPTATATTTPTATVTATVVMTTATMDLRDWLFLCYGLIAFLTEVIDSTTCPSVCRCDNGFIYCNDRG
LTSI PADIPDDATTLYLQNNQINNAGIPQDLKTKVNVQVIYLYVNDLDEFPINLPRSLRELHLQDNNVRTI
ARDSLARIPLLEKLHLDDNSVSTVSIEEDAFADSKQLKLLFLSRNILSSIPSGLPHTLEELRLDDNRISTI
PLHAFKGLNSLRRLVLDGNLLANQRIADDTFSRLQNLTELSLVRNSLAAPPLNLPSAHLQKLYLQDNAISB
IPYNTLAKMRELERLDLSNNNLTTLPRGLFDDLGNLAQLLLRNNPWFCGCNLMWLRDWVKARAAVVNVRGL
MCQGPEKVRGMAIKDITSEMDECFETGPQGGVANAAAKTTASNHASATTPQGSLFTLKAKRPGLRLPDSNI
DYPMATGDGAKTLAIHVKALTADSIRITWKATLPASSFRLSWLRLGHSPAVGSITETLVQGDKTEYLLTAL
EPKSTYIICMVTMETSNAYVADETPVCAKAETADSYGPTTTLNQEQNAGPMASLPLAGIIGGAVALVFLFL
VLGAICWYVHQAGELLTRERAYNRGSREKDDYMESGTKKDNSILEIRGPGLQMLPINPYRAKEEYVVHTIF
PSNGSSLCKATBTIGYGTTRGYRDGGIPDIDYSYT

A ClustalW comparison of the above protein sequences yields the following sequence alignment shown in Table 19B.

TABLE 19B
Comparison of the NOV19 protein sequences.

| | |
|---|---|
| NOV19a | ---MVVAHPTATATTTPTATVTATVVMTTATMDLRDWLFLCYGLIAFLTEVIDSTTCPSV |
| NOV19b | ----------------------------------------GSAAAPFTGSIDSTTCPSV |
| NOV19c | ------------------------------------------------------------ |
| NOV19d | ------------------------------------------------------------ |
| NOV19e | TGSMVVAHPTATATTTPTATVTATVVMTTATMDLRDWLFLCYGLIAFLTEVIDSTTCPSV |
| NOV19f | ---MVVAHPTATATTTPTATVTATVVMTTATMDLRDWLFLCYGLIAFLTEVIDSTTCPSV |
| NOV19g | ---MVVAHPTATATTTPTATVTATVVMTTATMDLRDWLFLCYGLIAFLTEVIDSTTCPSV |
| NOV19h | ------------------------------------------------------TTCPSV |
| NOV19i | ---MVVAHPTATATTTPTATVTATVVMTTATMDLRDWLFLCYGLIAFLTEVIDSTTCPSV |
| NOV19j | ---------TATATTTPTATVTATVVMTTATMDLRDWLFLCYGLIAFLTEVIDSTTCPSV |
| NOV19k | ---MVVAHPTATATTTPTATVTATVVMTTATMDLRDWLFLCYGLIAFLTEVIDSTTCPSV |
| NOV19a | CRCDNGFIYCNDRGLTSIPADIPDDATTLYLQNNQINNAGIPQDLKTKVNVQVIYLYEND |
| NOV19b | CRCDNGFIYCNDRGLTSIPADIPDDATTLYLQNNQINNAGIPQDLKTKVNVQVIYLYEND |
| NOV19c | --------------GSAAAPFTGSDATTLYLQNNQINNAGIPQDLKTKVNVQVIYLYEND |
| NOV19d | --------------GSAAAPFTGSDATTLYLQNNQINNAGIPQDLKTKVNVQVIYLYEND |
| NOV19e | CRCDNGFIYCNDRGLTSIPADIPDDATTLYLQNNQINNAGIPQDLKTKVNVQVIYLYEND |
| NOV19f | CRCDNGFIYCNDRGLTSIPADIPDDATTLYLQNNQINNAGIPQDLKTKVNVQVIYLYEND |
| NOV19g | CRCDNGFIYCNDRGLTSIPADIPDDATTLYLQNNQINNAGIPQDLKTKVNVQVIYLYEND |
| NOV19h | CRCDNGFIYCNDRGLTSIPADIPDDATTLYLQNNQINNACIPQDLKTKVNVQVIYLYEND |
| NOV19i | CRCDNGFIYCNDRGLTSIPADIPDDATTLYLQNNQINNAGIPQDLKTKVNVQVIYLYEND |
| NOV19j | CRCDNOFIYCNDRGLTSIPADIPDDATTLYLQNNQINNAGIPQDLKTKVNVQVIYLYEND |
| NOV19k | CRCDNGFIYCNDRGLTSIPADIPDDATTLYLQNNQINNAGIPQDLKTKVNVQVIYLYEND |
| NOV19a | LDEFPINLPRSLRELHLQDNNVRTIARDSLARIPLLEKLHLDDNSVSTVSIEEDAFADSK |
| NOV19b | LDEFFINLPRSLRELHLQDNNVRTIARDSLARIPLLEKLHLDDNSVSTVSIEEDAFADSK |
| NOV19c | LDEFPINLPRSLRELNLQDNNVRTIARDSLARIPLLEKLHLDDNSVSTVSIEEDAFADSK |
| NOV19d | LDEFPINLPRSLRELHLQDNNVRTIARDSLARIPLLEKLHLDDNSVSTVSIEEDAFADSK |
| NOV19e | LDEFPINLPRSLRELHLQDNNVRTIARDSLARIPLLEKLHLDDNSVSTVSIEEDAFADSK |
| NOV19f | LDEFPINLPRSLRELHLQDNNVRTIAROSLARIPLLEKLHLDDNSVSTVSIEEDAFADSK |
| NOV19g | LDEFPINLPRSLRELHLQDNNVRTIARDSLARIPLLEKLHLDDNSVSTVSIEEDAFADSK |
| NOV19h | LDEFPINLPRSLRELHLQDNNVRTIARDSLARIPLLEKLHLDDNSVSTVSIEEDAFADSK |
| NOV19i | LDEFPINLPRSLRELHLQDNNVRTIARDSLARIPLLEKLHLDDNSVSTVSIEEDAFADSK |
| NOV19j | LDEFPINLPRPLRELHLQDNNVRTIARDSLARIPLLEKLHLDDNSVSTVSIEEDAFADSK |
| NOV19k | LDEFPINLPRSLRELHLQDNNVRTIARDSLARIPLLEKLHLDDNSVSTVSIEEDAFADSK |
| NOV19a | QLKLLFLSRNHLSSIPSGLPHTLEELRLDDNRISTIPLHAFKGLNSLRRLVLDGNLLANQ |
| NOV19b | QLKLLFLSRNHLSSIPSGLPHTLEELRLDDNRISTIPLHAFKGLNSLRRLVLDGNLLANQ |
| NOV19c | QLKLLFLSRNHLSSIPSGLPNTLEELRLDDNRISTIPLHAFKGLNSLRRLVLDGNLLANQ |
| NOV19d | QLKLLFLSRNHLSSIPSGLPHTLEELRLDDNRISTIPLHAFKGLNSLRRLVLDGNLLANQ |
| NOV19e | QLKLLFLSRNHLSSIPSGLPNTLEELRLDDNRISTIPLHAFKGLNSLRRLVLDGNLLANQ |
| NOV19f | QLKLLFLSRNHLSSIPSGLPHTLEELRLDDNRISTIPLHAFKGLNSLRRLVLDGNLLANQ |
| NOV19g | QLKLLFLSRNHLSSIPSGLPHTLEELRLDDNRISTIPLHAFKGLNSLRRLVLDGNLLANQ |
| NOV19h | QLKLLFLSRNHLSSIPSGLPHTLEELRLDDNRISTIPLHAFKGLNSLRRLVLDGNLLANQ |
| NOV19i | QLKLLFLSRNHLSSIPSGLPHTLEELRLDDNRISTIPLHAFKGLNSLRRLVLDGNLLANQ |
| NOV19j | QLKLLFLSRNHLSSIPSGLPHTLEELRLDDNRISTIPLHAFKGLNSLRRLVLDGNLLANQ |
| NOV19k | QLKLLFLSRNHLSSIPSGLPHTLEELRLDDNRISTIPLHAFKGLNSLRRLVLDGNLLANQ |
| NOV19a | RIADDTFSRLQNLTELSLVRNSLAAPPLNLPSAHLQKLYLQDNAISHIPYNTLAKMRELE |
| NOV19b | RIADDTFSRLQNLTELSLVRNSLAAPPLNLPSAHLQKLYLQDNAISHIPYNTLAKMRELE |
| NOV19c | RIADDTFSRLQNLTELSLVRNSLAAPPLNLPSAHLQKLYLQDNAISHIPYNTLAKMRELE |
| NOV19d | RIADDTFSRLQNLTELSLVRNSLAAPPLNLPSAHLQKLYLQDNAISHIPYNTLAKMRELE |
| NOV19e | RIADDTFSRLQNLTELSLVRNSLAAPPLNLPSAHLQKLYLQDNAISHIPYNTLAKMRELE |
| NOV19f | RIADDTFSRLQNLTELSLVRNSLAAPPLNLPSAHLQKLYLQDNAISHIPYNTLAKMRELE |
| NOV19g | RIADDTFSRLQNLTELSLVRNSLAAPPL----------YLQDNAISHIPYNTLAKMRELE |
| NOV19h | RIADDTFSRLQNLTELSLVRNSLAAPPLNLPSAHLQKLYLQDNAISHIPYNTLAKMRELE |
| NOV19i | RIADDTFSRLQNLTELSLVRNSLAAPPLNLPSAHLQKLYLQDNAISHIPYNTLAKMRELE |
| NOV19j | RIADDTFSRLQNLTELSLVRNSLAAPPLNLPSAHLQKLYLQDNAISHIPYNTLAKMRELE |
| NOV19k | RIADDTFSRLQNLTELSLVRNSLAAPPLNLPSAHLQKLYLQDNAISHIPYNTLAKMRELE |
| NOV19a | RLDLSNNNLTTLPRGLFDDLGNLAQLLLRNNPWFCGCNLMWLRDWVKARAAVVNVRGLMC |
| NOV19b | RLDLSNNNLTTLPRGLFDDLGNLAQLLLRNNFWFCGCNLMWLRDWVKARAAVVNVRGLMC |
| NOV19c | RLDLSNNNLTTLPRGLFDDLG-LEGKGGRADPAFLY |
| NOV19d | RLDLSNNNLTTLPRGLFDDLG-NLAQLLLRNNPWFCGCNLMWLRDWVKARAAVVNVRGLM |
| NOV19e | RLDLSNNNLTTLPRGLFDDLGNLAQLLLRNNPWFCGCNLMWLRDWVKARAAVVNVRGLMC |
| NOV19f | RLDLSNNNLTTLPRGLFDDLGNLAQLLLRNNPWFCGCNLMWLRDWVKARAAVVNVRGLMC |
| NOV19g | RLDLSNNNLTTLPRGLFDDLGNLAQLLLRNNPWFCGCNLMWLRDWVKARAAVVNVRGLMC |
| NOV19h | RLDLSNNNLTTLPRGLFDDLGNLAQLLLRNNPWFCGCNLMWLRDWVKARAAVVNVRGLMC |
| NOV19i | RLDLSNNNLTTLPRGLFDDLGNLAQLLLRNNPWFCGCNLMWLRDWVKARAAVVNVRGLMC |
| NOV19j | RLDLSNNNLTTLPRGLFDDLGNLAQLLLRNNPWFCGCNLMWLRDWVKARAAVVNVRGLMC |
| NOV19k | RLDLSNNNLTTLPRGLFDDLGNLAQLLLRNNPWFCGCNLMWLRDWVKARAAVVNVRGLMC |
| NOV19a | QGPEKVRGMAIKDITSEMDECFETGPQGGVANAAAKTTASNHASATTPQGSLFTLKAKRP |
| NOV19b | QGPEKVRGMAIKDITSEMDECFETGPQGGVANAAAKTTASNHASATTPQGSLFTLKAKRP |
| NOV19c | |
| NOV19d | CQGPEKVRGMAIKDITSEMDECFETGPQGGVANAAAKTTASNHASATTPQGSLFTLKAKR |
| NOV19e | QGPEKVRGMAIKDITSEMDECFETGPQGGVANAAAKTTASNHASATTPQGSLFTLKAKRP |
| NOV19f | QGPEKVRGMAIKDITSEMDECFETGPQGGVANAAAKTTASNHASATTPQGSLFTLKAKRP |
| NOV19g | QGPEKVRGMAIKDITSEVESVLRRAPQGGVANAAAKTTASNHASATTPQGSLFTLKAKRP |
| NOV19h | QGPEKVRGMAIKDITSEMDECFETGPQGGVANAAAKTTASNHASATTPQGSLFTLKAKRP |
| NOV19i | QGPEKVRGMAIKDITSEMDECFETGPQGGVANAAAKTTASNHASATTPQGSLFTLKAKRP |
| NOV19j | QGPEKVRGMAIKDITSEMDECFETGPQGGVANAAAKTTASNHATATTPQGSLFTLKAKRP |
| NOV19k | QGPEKVRGMAIKDITSEMDECFETGPQGGVANAAAKTTASNHASATTPQGSLFTLKAKRP |
| NOV19a | GLRLPDSNIDYPMATGDGAKTLAIHVKALTADSIRITWKATLPASSFRLSWLRLGHSPAV |
| NOV19b | GLRLPDSNIDYPMATGDGAKTLAIHVKALTADSIRITWKATLPASSFRLSWLRLGHSPAV |
| NOV19c | |
| NOV19d | PGLRLPDSNIDYPMATGDGAKTLAIHVKALTADSIRITWKATLPASSFRLSWLRLGHSPA |
| NOV19e | GLRLPDSNIDYPMATGDGAKTLAIHVKALTADSIRITWKATLPASSFRLSWLRLGHSPAV |
| NOV19f | GLRLPDSNIDYPMATGDGAKTLAIHVKALTADSIRITWKATLPASSFRLSWLRLGHSPAV |
| NOV19g | GLRLPDSNIDYPMATGDGAKTLAIHVKALTADSIRITWKATLPASSFRLSWLRLGHSPAV |
| NOV19h | GLRLPDSNIDYPMATGDGAKTLAIHVKALTADSIRITWKATLPASSFRLSWLRLGHSPAV |
| NOV19i | GLRLPDSNIDYPMATGDGAKTLAIHVKALTADSIRITWKATLPASSFRLSWLRLGHSPAV |
| NOV19j | GLRLPDSNIDYPMATGDGAKTLAIHVKALTADSIRITWKATLPASSFRLSWLRLCHSPAV |
| NOV19k | GLRLPDSNIDYPMATGDGAKTLAIHVKALTADSIRITWKATLPASSFRLSWLRLGHSPAV |
| NOV19a | GSITETLVQGDKTEYLLTALEPKSTYIICMVTMETSNAYVADETPVCAKAETADSYGPTT |
| NOV19b | GSITETLVQGDKTEYLLTALEPKSTYIICMVTMETSNAYVADETPVCAKAETADSYGPTT |
| NOV19c | |
| NOV19d | VGSITETLVQGDKTEYLLTALEPKSTYIICMVTMETSNAYVADETPVCAKAETADSYGPT |
| NOV19e | GSITETLVQGDKTEYLLTALEPKSTYIICMVTMETSNAYVADETPVCAKAETADSYGPTT |
| NOV19f | GSITETLVQGDKTEYLLTALEPKSTYIICMVTMETSNAYVADETPVCAKAETADSYGPTT |
| NOV19g | GSITETLVQGDKTEYLLTALEPKSTYIICMVTMETSNAYVADETPVCAKAETADSYGPTT |
| NOV19h | GSITETLVQGDKTEYLLTALEPKSTYIICMVTMETSNAYVADETPVCAKAETADSYGPTT |
| NOV19i | GSITETLVQGDKTEYLLTALEPKSTYIICMVTMETSNAYVADETPVCAKAETADSYGPTT |
| NOV19j | GSITETLVQGDKTEYLLTALEPKSTYIICMVTMETSNAYVADETPVCAKAETADSYGPTT |
| NOV19k | GSITETLVQGDKTEYLLTALEPKSTYIICMVTMETSNAYVADETPVCAKAETADSYGPTT |
| NOV19a | TLNQEQNAGPMASLPLAGIIGGAVALVFLFLVLGAICWYVHQAGELLTRERAYNRGSREK |
| NOV19b | TLNQEQNAGPMASLPLAGIIGGAVALVFLFLVLGAICWYVHQAGELLTRERAYNRGSRKK |
| NOV19c | |
| NOV19d | TTLNQEQNAGPMASLPLAGIIGGAVALVFLFLVLGAICWYVHQAGELLTRERAYNRGSRK |
| NOV19e | TLNQEQNAGPMASLPLAGIIGGAVALVFLFLVLGAICWYVHQAGELLTRERAYNRGSRKK |
| NOV19f | TLNQEQNAGPMASLPLAGIIGGAVALVFLFLVLGAICWYVHQAGELLTRERAYNRGSRKK |
| NOV19g | TLNQEQNAGPMASLPLAGIIGGAVALVFLFLVLGAICWYVHQAGELLTRERAYNRGSRKK |
| NOV19h | TLNQEQNAG |
| NOV19i | TLNQEQNAGPMASLPLAGIIGGAVALVFLFLVLGAICWYVHQAGELLTRERAYNRGSRKK |
| NOV19j | TLNQEQNAGPMASLPLAGIIGGAVALVFLFLVLGAICWYVHQAGELLTRERAYNRGSRKK |
| NOV19k | TLNQEQNAGPMASLPLAGIIGGAVALVFLFLVLGAICWYVNQAGELLTRERAYNRGSRKK |
| NOV19a | DDYMESGTKKDNSILEIRGPGLQMLPINPYRAKEEYVVHTIFPSNGSSLCKATHTIGYGT |
| NOV19b | DDYMESGTKKDNSILEIRGPGLQMLPINPYRAKEEYVVHTIFPSNGSSLCKATHTIGYGT |
| NOV19c | |
| NOV19d | KDDYMESGTKKDNSILEIRGPGLQMLPINPYRAKEEYVVHTIFPSNGSSLCKATHTIGYG |
| NOV19e | DDYMESGTKKDNSILEIRGPGLQMLPINPYRAKEEYVVHTIFPSNGSSLCKATHTIGYGT |
| NOV19f | DDYMESGTKKDNSILEIRGPGLQMLPINPYRAKEEYVVNTIFPSNGSSLCKATHTIGYGT |
| NOV19g | DDYMESGTKKDNSILEIRGPGLQMLPINPYRAKEEYVVHTIFPSNGSSLCKATHTIGYGT |
| NOV19h | |
| NOV19i | DDYMESGTKKDNSILEIRGPGLQMLPINPYRAKEEYVVHTIFPSNGSSLCKATHTIGYGT |
| NOV19j | DDYMESGTKKDNSILEIRGPGLQMLPINPYRAKEEYVVHTIFPSNGSSLCKATHTIGYGT |
| NOV19k | DDYMESGTKKDNSILEIRGPGLQNLPINPYRAKEEYVVHTIFPSNGSSLCKATHTIGYGT |
| NOV19a | TRGYRD-GGIPDIDYSYT |
| NOV19b | TRGYRD-GGIPDIDYSYTLEGKGGRA |
| NOV19c | |
| NOV19d | TTRGYRDGGIPDIDYSYTLEGKGGRPDPAFLYTAGIIRSHC |
| NOV19e | TRGYRD-GGIPDIDYSYTLEG |
| NOV19f | TRGYRD-GGIPDIDYSYT |
| NOV19g | TRGYRD-GGIPDIDYSYT |
| NOV19h | |
| NOV19i | TRGYRD-GGIPDIDYSYT |
| NOV19j | TRGYRHCHHLPDIDYSYTRSPT |
| NOV19k | TRGYRD-GGIPDIDYSYT |
| NOV19a | (SEQ ID NO: 296) |
| NOV19b | (SEQ ID NO: 298) |
| NOV19c | (SEQ ID NO: 300) |
| NOV19d | (SEQ ID NO: 302) |
| NOV19e | (SEQ ID NO: 304) |
| NOV19f | (SEQ ID NO: 306) |
| NOV19g | (SEQ ID NO: 308) |
| NOV19h | (SEQ ID NO: 310) |
| NOV19i | (SEQ ID NO: 312) |
| NOV19j | (SEQ ID NO: 314) |
| NOV19k | (SEQ ID NO: 316) |

Further analysis of the NOV19a protein yielded the following properties shown in Table 19C.

TABLE 19C
Protein Sequence Properties NOV19a

| SignalP | Cleavage site between residues 52 and 53 | | | | |
|---|---|---|---|---|---|
| analysis: | | | | | |
| PSORT II | PSG: a new signal peptide prediction method | | | | |
| analysis: | N-region: length 0; pos. chg 0; neg. chg 0 | | | | |
| | H-region: length 29; peak value 8.99 | | | | |
| | PSG score: 4.59 | | | | |
| | GvH: von Heijne's method for signal seq. recognition | | | | |
| | GvH score (threshold: −2.1): −3.42 | | | | |
| | possible cleavage site: between 53 and 54 | | | | |
| | >>> Seems to have no N-terminal signal peptide | | | | |
| | ALOM: Klein et al's method for TM region allocation | | | | |
| | Init position for calculation: 1 | | | | |
| | Tentative number of TMS(s) for the threshold 0.5: 2 | | | | |
| | Number of TMS(s) for threshold 0.5: 1 | | | | |
| | INTEGRAL | Likelihood = | −10.46 | Transmembrane | 553-569 |
| | PERIPHERAL | Likelihood = | 4.93 (at 493) | | |

| ALOM score: −10.46 (number of TMSs: 1) | | | |
|---|---|---|---|
| MTOP: Prediction of membrane topology (Hartmann et al.) | | | |
| Center position for calculation: 560 | | | |
| Charge difference: 4.5 C(1.5)-N(−3.0) | | | |
| C > N: C-terminal side will be inside | | | |
| >>>Caution: Inconsistent mtop result with signal peptide | | | |
| >>> Single TMS is located near the C-terminus | | | |
| >>> membrane topology: type Nt (cytoplasmic tail 1 to 552) | | | |
| MITDISC: discrimination of mitochondrial targeting seq | | | |
| R content: | 0 | Hyd Moment (75): | 3.83 |
| Hyd Moment(95): | 1.63 | G content: | 0 |
| D/E content: | 1 | S/T content: | 12 |

```
Score: −2.21
Gavel: prediction of cleavage sites for mitochondrial preseq
cleavage site motif not found
NUCDISC: discrimination of nuclear localization signals
pat4: none
pat7: none
bipartite: none
content of basic residues: 9.2%
NLS Score: −0.47
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: too long tail
Dileucine motif in the tail: found
LL at 152
LL at 181
LL at 233
LL at 323
LL at 324
checking 63 PROSITE DNA binding motifs:
Leucine zipper pattern (PS00029): *** found ***
LFLSRNHLSSIPSGLPHTLEEL at 182
LDLSNNNLTTLPRGLFDDLGNL at 299
none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: nuclear
Reliability: 55.5
COIL: Lupas's algorithm to detect coiled-coil regions
total: 0 residues
Final Results (k = 9/23):
30.4%: nuclear
26.1%: cytoplasmic
13.0%: Golgi
13.0%: mitochondrial
  8.7%: endoplasmic reticulum
  4.3%: vesicles of secretory system
  4.3%: peroxisomal
>> prediction for CG54254-04 is nuc (k = 23)
```

A search of the NOV19a protein against the Geneseq database, a proprietary database that contains sequences published in patents and patent publication, yielded several homologous proteins shown in Table 19D.

TABLE 19D
Geneseq Results for NOV19a

| | | NOV19a | Identities/ | |
|---|---|---|---|---|
| | | Residues/ | Similarities for | |
| Geneseq | Protein/Organism/Length [Patent | Match | the Matched | Expect |
| Identifier | #, Date] | Residues | Region | Value |
| AAE23800 | Proteoglycan-like (NOV3) protein - | 1 . . . 674 | 673/674 (99%) | 0.0 |
| | Unidentified, 674 aa. | 1 . . . 674 | 674/674 (99%) | |
| | [WO200230979-A2, 18 APR. 2002] | | | |
| AAU00692 | Proteoglycan-like protein - Homo | 1 . . . 674 | 673/674 (99%) | 0.0 |
| | sapiens, 674 aa. [WO200129217- | 1 . . . 674 | 674/674 (99%) | |
| | A2, 26 APR. 2001] | | | |
| AAU12189 | Human PRO1483 polypeptide | 1 . . . 674 | 673/674 (99%) | 0.0 |
| | sequence - Homo sapiens, 674 aa. | 1 . . . 674 | 674/674 (99%) | |
| | [WO200140466-A2, 07 JUN. 2001] | | | |
| AAM40226 | Human polypeptide SEQ ID NO | 1 . . . 674 | 673/674 (99%) | 0.0 |
| | 3371 - Homo sapiens, 674 aa. | 1 . . . 674 | 674/674 (99%) | |
| | [WO200153312-A1, 26 JUL. 2001] | | | |
| AAE23802 | Fibromodulin-like (NOV5) protein - | 1 . . . 674 | 655/674 (97%) | 0.0 |
| | Unidentified, 664 aa. | 1 . . . 664 | 658/674 (97%) | |
| | [WO200230979-A2, 18 APR. 2002] | | | |

In a BLAST search of public sequence databases, the NOV19a protein was found to have homology to the proteins shown in the BLASTP data in Table 19E.

TABLE 19E
Public BLASTP Results for NOV19a

| | | NOV19a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q8WVA2 | Hypothetical protein - Homo sapiens | 1 . . . 674 | 673/674 (99%) | 0.0 |
| | (Human), 674 aa. | 1 . . . 674 | 674/674 (99%) | |
| Q9NZU1 | Leucine-rich repeat transmembrane | 29 . . . 674 | 646/646 (100%) | 0.0 |
| | protein FLRT1 precursor | 1 . . . 646 | 646/646 (100%) | |
| | (Fibronectin-like domain-containing | | | |
| | leucine-rich transmembrane protein | | | |
| | 1) - Homo sapiens (Human), 646 aa. | | | |
| Q9NZU0 | Leucine-rich repeat transmembrane | 53 . . . 674 | 377/627 (60%) | 0.0 |
| | protein FLRT3 precursor | 30 . . . 649 | 465/627 (74%) | |
| | (Fibronectin-like domain-containing | | | |
| | leucine-rich transmembrane protein | | | |
| | 3) - Homo sapiens (Human), 649 aa. | | | |
| CAC33411 | Sequence 5 from Patent WO0110902 | 53 . . . 674 | 377/627 (60%) | 0.0 |
| | - Homo sapiens (Human), 649 aa. | 30 . . . 649 | 464/627 (73%) | |
| Q8BGT1 | Fibronectin leucine rich | 53 . . . 674 | 375/627 (59%) | 0.0 |
| | transmembrane protein 3 homolog - | 30 . . . 649 | 462/627 (72%) | |
| | Mus musculus (Mouse), 649 aa. | | | |

PFam analysis predicts that the NOV19a protein contains the domains shown in the Table 19F.

TABLE 19F
Domain Analysis of NOV19a

| | | Identities/ | |
|---|---|---|---|
| | NOV19a | Similarities | |
| | Match | for the | Expect |
| Pfam Domain | Region | Matched Region | Value |
| LRRNT | 53 . . . 80 | 12/35 (34%) | 1.1e−05 |
| | | 19/35 (54%) | |
| LRR | 128 . . . 151 | 8/25 (32%) | 0.11 |
| | | 21/25 (84%) | |
| LRR | 178 . . . 197 | 10/25 (40%) | 0.38 |
| | | 18/25 (72%) | |
| LRR | 199 . . . 222 | 10/25 (40%) | 0.0026 |
| | | 23/25 (92%) | |
| LRR | 271 . . . 294 | 5/25 (20%) | 0.056 |
| | | 22/25 (88%) | |
| LRR | 295 . . . 318 | 13/25 (52%) | 0.00046 |
| | | 21/25 (84%) | |
| LRRCT | 328 . . . 379 | 15/54 (28%) | 8.2e−13 |
| | | 43/54 (80%) | |
| fn3 | 435 . . . 513 | 16/88 (18%) | 0.12 |
| | | 54/88 (61%) | |

### Example 20

The NOV20 clone was analyzed, and the nucleotide and encoded polypeptide sequences are shown in Table 20A.

TABLE 20A
NOV20 Sequence Analysis

| | | |
|---|---|---|
| NOV2Oa, CG96778-02 | SEQ ID NO: 325 | 1365 bp |
| DNA Sequence | ORF Start: ATG at 1 | ORF Stop: TAA at 1363 |

ATGGCAGCGGGGTTCGGGCGATGCTGCAGGGTCCTGAGAAGTATTTCTCGTTTTCATTGGAGATCACA
GCATACAAAAGCCAATCGACAACGTGAACCAGGATTAGGATTTAGTTTTGAGTTCACCGAACAGCAGA
AAGAATTTCAAGCTACTGCTCGTAAATTTGCCAGAGAGGAAATCATCCCAGTGGCTGCAGAATATGAT
AAAACTGGTGAATATCCAGTCCCCCTAATTAGAAGAGCCTGGGAACTTGGTTTAATGAACACACACAT
TCCAGAGAACTGTGACTACAGTGTTTGCCCACTTTTGGAAGCTTGCACTCTATACCTAGATGCGTTTT
TCCTTCTTCTAACTGGTTCCAACCTTAACTTGCACCTAAACCTTGGAGGTCTTGGACTTGGAACTTTT
GATGCTTCTTTAATTAGTGAAGAATTGGCTTATGGATGTACAGGGGTTCAGACTGCTATTGAAGGAAA
TTCTTTGGGGCAAATGCCTATTATTATTGCTGGAAATGATCAACAAAAGAAGAAGTATTTGGGGAGAA
TGACTGAGGAGCCATTGATGTGTGCTTATTGTGTAACAGAACCTGGAGCAGGCTCTGATGTAGCTGGT
ATAAAGACCAAAGCAGAAAAGAAAGGAGATGAGATATTATTAATGGTCAGAAGATGTGGATAACCAA
CGGAGGAAAAGCTAATTGGTATTTTTTATTGGCACGTTCTGATCCAGATCCTAAAGCTCCTGCTAATA
AAGCCTTTACTGGATTCATTGTGGAAGCAGATACCCCAGGAATTCAGATTGGGAGAAAGGAATTAAAC
ATGGGCCAGCGATGTTCAGATACTAGAGGAATTGTCTTCGAAGATGTGAAAGTGCCTAAAGAAAATGT
TTTAATTGGTGACGGAGCTGGTTTCAAAGTTGCAATGGGAGCTTTTGATAAAACCAGACCTGTAGTAG
CTGCTGGTGCTGTTGGATTAGCACAAAGAGCTTTGGATGAAGCTACCAAGTATGCCCTGGAAAGGAAA
ACTTTCGGAAAGCTACTTGTAGAGCACCAAGCAATATCATTTATGCTGGCTGAAATGGCAATGAAGT
TGAACTAGCTAGAATGAGTTACCAGAGAGCAGCTTGGGAGGTTGATTCTGGTCGTCGAAATACCTATT
ATGCTTCTATTGCAAAGGCATTTGCTGGAGATATTGCAAATCAGTTAGCTACTGATGCTGTGCAGATA
CTTGGAGGCAATGGATTTAATACAGAATATCCTGTAGAAAAACTAATGAGGGATGCCAAAATCTATCA
GATTTATGAAGGTACTTCACAAATTCAAAGACTTATTGTAGCCCGTGAACACATTGACAAGTACAAAA
ATTAA

| | | |
|---|---|---|
| NOV20a, CG96778-02 | | |
| Protein Sequence | SEQ ID NO: 326 454 aa | MW at 50270.1kD |

MAAGFGRCCRVLRSISRFHWRSQHTKANRQREPGLGFSFEFTEQQKEFQATARKFAREEIIPVAAEYD
KTGEYPVPLIRRAWELGLMNTHIPENCDYSVCPLLEACTLYLDAFFLLLTGSNLNLHLNLGGLGLGTF
DACLISEELAYGCTGVQTAIEGNSLGQMPIIIAGNDQQKKKYLGRMTEEPLMCAYCVTEPGAGSDVAG
IKTKAEKKGDEYIINGQKMWITNGGKANWYFLLARSDPDPKAPANKAFTGFIVEADTPGIQIGRKELN
MGQRCSDTRGIVFEDVKVPKENVLIGDGAGFKVAMGAFDKTRPVAAGAVGLAQRALDEATKYALERK
TFGKLLVEHQAISFMLAEMAMKVELARMSYQRAAWEVDSGRRNTYYASIAKAFAGDIANQLATDAVQI
LGGNGFNTEYPVEKLMRDAKIYQIYEGTSQIQRLIVAREHIDKYYN

| | | |
|---|---|---|
| NOV20b, CG96778-01 | SEQ ID NO: 327 | 3387 bp |
| DNA Sequence | ORF START: ATG at 1387 | ORF Stop: TAA at 2650 |

CTGCAGGACAGACAAACAAGGGGGTAGCTTGCTTGGGTGAATGGTGGCAGGAACTACCGACTAGACAT
GTTTAAGATGAGGGCTCCATCTTCGCTTCTCTGCCAGCCACGTGTACAGTAAGAAGGGGTTACAATAG
GCATATGGGTGATTTTGTGCTTTTCGTTCATCTTTTCTGTGTTTAAAATGTTCAGAATAAGAAATTGG
AACAAAGGAGACATGAATAGACAATTCCTAATCATCTTTAAGAGTCCGCTTTGTGTTTTCATTACACA
CCACCACTTTGAAGGCTTTCTTGCCTATACCAGGACAAACTCAGCTCTTTATCCCTTTTCCGAATTTT
CCTGGTACTTTCACTTTGAATATAGCGCTTAATTAACATTCTGCCTTGTACCTAGGACTAACACACTA
TAAATTCCCAGAAGACAAAGTAGGGGAATACAATAACAGGATATAGAATTTTAACAGCTAAATTAGAT
GAATTTATGGGTGACCTTTATTGGGCAAAAGAAAATGTTAAGTTAGTATAAGATTTAGTATAAGCTAC
CACTCAAAACTCAGGGTCTCACTGGAAGAGAAAGTGACTCCAGGTAGAATTCCTCAGGGAGACATTCA
CTTCCATCATTCGCTGAACCAGGAGCTTTGGACAGCCTCGGATTGCACCCGCATATCCAAGGACACCA
CATCAGCGGACAAGTCATAAACAGCCTTGGGAATACGCGGAAAGGTCAAATTTACCTAAACAATTAAA
TTCTCTTTTAAATTTTAAGGAAACACAAGTATGCTTTCGCTTTAGGTAGGGCATTTGAGAGCAAAATG
TACTAATACTTTGAATCCGCCAAGCAGACACGATCTGGGTTTGACCTTTCTCTCCGGGTAAAGGTGAA
GGCTGACCACGGGGCCGCTCTCCCTCCAGCCCCAGCCACGCCCTCTAACCCAGGTTCCCGTCCTGCAC
CGCGCCGCAACTCCCCCCACCGTTCAGCGCAACCGGCCCTCCCAGCCCCGCCGCCGTCCCCCTCCCCG
CCCTGGCTCTCTTTCCGCGCTGCGTCAGCCTCGGCGTCCCACAGAGAGGGCCAGAGGTGGAAACGCAG
AAAACCAAACCAGGACTATCAGAGATTGCCCGGAGAGGGGATGCGACCCCTCCCCAGGTCGCAGCGAC
GGCGCACGCAAGGGTCACGGAGCATGCGTTGGCTATCCGGCGCCGGGGACCGCTGCCACCCCGCCTAG
CGCAGCGCCCCGTCCTTCCGCAGCCCAACCGCCTCTTCCCGCCCCGCCCCATCCCGCCCCACGGGCTC
CAGTGGGCGGGACCAGAGGAGTCCCGCGTTCGGGGAGTATGTCAAGGCCGTGACCCGTGTATTATTGT
CCAGTGGCCGGAACGGAGAGCCAACATGGCAGCGGGGTTCGGGCGATGCTGCAGGGTCCTGAGAAGT
ATTTCTCGTTTTCATTGGAGATCACAGCATACAAAAGCCAATCGACAACGTGAACCAGGATTAGGATT
TAGTTTTGAGTTCACCGAACAGCAGAAAGAATTTCAAGCTACTGCTCGTAAATTTGCCAGAGAGGAAA
TCATCCCAGTGGCTGCAGAATATGATAAAACTGGTGAATATCCAGTCCCCCTAATTAGAAGAGCCTGG
GAACTTGGTTTAATGAACACACACATTCCAGAGAACTGTGGAGGTCTTGGACTTGGAACTTTTGATGC
TTGTTTAATTAGTGAAGAATTGGCTTATGGATGTACAGGGGTTCAGACTGCTATTGAAGGAAATTCTT
TGGGGCAAATGCCTATTATTATTGCTGGAAATGATCAACAAAAGAAGAAGTATTTGGGGAGAATGACT
GAGGAGCCATTGATGTGTGCTTATTGTGTAACAGAACCTGGAGCAGGCTCTGATGTAGCTGGTATAAA
GACCAAAGCAGAAAAGAAAGGAGATGAGTATATTATTAATGGTCAGAAGATGTGGATAACCAACGGAG
GAAAAGCTAATTGGTATTTTTTATTGGCACGTTCTGATCCAGATCCTAAAGCTCCTGCTAATAAAGCC
TTTACTGGATTCATTGTGGAAGCAGATACCCCAGGAATTCAGATTGGGAGAAAGGAATTAAACATGGG
CCAGCGATGTTCAGATACTAGAGGAATTGTCTTCGAAGATGTGAAAGTGCCTAAAGAAAATGTTTTAA
TTGGTGACGGAGCTGGTTTCAAAGTTGCAATGGGAGCTTTTGATAAAACCAGACCTGTAGTAGCTGCT
GGTGCTGTTGGATTAGCACAAAGAGCTTTGGATGAAGCTACCAAGTATGCCCTGGAAAGGAAAACTTT
CGGAAAGCTACTTGTAGAGCACCAAGCAATATCATTTATGCTGGCTGAAATGGCAATGAAAGTTGAAC
TAGCTAGAATGAGTTACCAGAGAGCAGCTTGGGAGGTTGATTCTGGTCGTCGAAATACCTATTATGCT
TCTATTGCAAAGGCATTTGCTGGAGATATTGCAAATCAGTTAGCTACTGATGCTGTGCAGATACTTGG
AGGCAATGGATTTAATACAGAATATCCTGTAGAAAAACTAATGAGGGATGCCAAAATCTATCAGATTT
ATGAAGGTACTTCACAAATTCAAAGACTTATTGTAGCCCGTGAACACATTGACAAGTACAAAAATTAA
AAAAATTACTGTAGAAATATTGAATAACTAGAACACAAGCCACTGTTTCAGCTCCAGAAAAAAGAAAG
GGCTTTAACGTTTTTTCCAGTGAAAACAAATCCTCTTATATTAAATCTAAGCAACTGCTTATTATAGT
AGTTTATACTTTTGCTTAACTCTGTTATGTCTCTTAAGCAGGTTTGGTTTTTATTAAAATGATGTGTT
TTCTTTAGTACCACTTTACTTGAATTACATTAACCTAGAAAACTACATAGGTTATTTTGATCTCTTAA
GATTAATGTAGCACAAATTTCTTGGAATTTTATTTTTGTAATGACAGAAAAGTGGGCTTAGAAAGTAT
TCAAGATGTTACAAAATTTACATTTAGAAAATATTGTAGTATTTGAATACTGTCAACTTGACAGTAAC
TTTGTAGACTTAATGGTATTATTAAAGTTCTTTTATTGCAGTTTGGAAAGCATTTGTGAAACTTTCT
GTTTGGCACAGAAACAGTCAAAATTTTGACATTCATATTCTCCTATTTTACAGCTACAAGAACTTTCT
TGAAAATCTTATTTAATTCTGAGCCCATATTTCACTTACCTTATTTAAAATAAATCAATAAAGCTTGC
CTTAAATTATTTTTATATGACTGTTGGTCTCTAGGTAGCCTTTGGTCTATTGTACACAATCTCATTTC
ATATGTTTGCATTTTGGCAAAGAACTTAATAAAATTGTTCAGTGCTTATTATCAT

| | | | |
|---|---|---|---|
| NOV20b, CG96778-01 | | | |
| Protein Sequence | SEQ ID NO: 328 | 421 aa | MW at 46587.9kD |

MAAGFGRCCRVLRSISRFHWRSQHTKANRQREPGLGFSFEFTEQQKEFQATARKFAREEIIPVAAEYD
KTGEYPVPLIRRAWELGLMNTHIPENCGGLGLGTFDACLISEELAYGCTGVQTAIEGNSLGQMPIIIA
GNDQQKKKYLGRMTEEPLMCAYCVTEPGAGSDVAGIKTKAEKKGDEYIINGQKMWITNGGKANWYFLL
ARSDPDPKAPANKAFTGFIVEADTPGIQIGRKELNMGQRCSDTRGIVFEDVKVPKENVLIGDGAGFKV
AMGAFDKTRPVVAAGAVGLAQRALDEATKYALERKTFGKLLVEHQAISFMLAEMAMKVELARMSYQRA
AWEVDSGRRNTYYASIAKAFAGDIANQLATDAVQILGGNGFNTEYPVEKLMRDAKIYQIYEGTSQIQR
LIVAREHIDKYKN

| | | |
|---|---|---|
| NOV20c, 276657466 | SEQ ID NO: 329 | 1288 bp |
| DNA Sequence | ORF Start: at 2 | ORF Stop: end of sequence |

CACCAGATCTCCCACCATGGCAGCGGGGTTCGGGCGATGCTGCAGGGTCCTGAGAAGTATTTCTCGTT
TTCATTGGAGATCACAGCATACAAAAGCCAATCGACAACGTGAACCAGGATTAGGATTTAGTTTTGAG
TTCACCGAACAGCAGAAAGAATTTCAAGCTACTGCTCGTAAATTTGCCAGAGAGGAAATCATCCCAGT
GGCTGCAGAATATGATAAAACTGGTGAATATCCAGTCCCCCTAATTAGAAGAGCCTGGGAACTTGGTT
TAATGAACACACACATTCCAGAGAACTGTGGAGGTCTTGGACTTGGAACTTTTGATGCTTGTTTAATT
AGTGAAGAATTGGCTTATGGATGTACAGGGGTTCAGACTGCTATTGAAGGAAATTCTTTGGGGCAAT
GCCTATTATTATTGCTGGAAATGATCAACAAAAGAAGAAGTATTTGGGGAGAATGACTGAGGAGCCAT
TGATGTGTGCTTATTGTGTAACAGAACCTGGAGCAGGCTCTGATGTAGCTGGTATAAAGACCAAAGCA
GAAAAGAAAGGAGATGAGTATATTATTAATGGTCAGAAGATGTGGATAACCAACGGAGGAAAAGCTAA
TTGGTATTTTTTATTGGCACGTTCTGATCCAGATCCTAAAGCTCCTGCTAATAAAGCCTTTACTGGAT
TCATTGTGGAAGCAGATACCCCAGGAATTCAGATTGGGAGAAAGGAATTAAACATGGGCCAGCGATGT
TCAGATACTAGAGGAATTGTCTTCGAAGATGTGAAAGTGCCTAAAGAAAATGTTTTAATTGGTGACGG
AGCTGGTTTCAAAGTTGCAATGGGAGCTTTTGATAAAACCAGACCTGTAGTAGCTGCTGGTGCTGTTG
GATTAGCACAAAGAGCTTTGGATGAAGCTACCAAGTATGCCCTGGAAAGGAAAACTTTCGGAAAGCTA
CTTGTAGAGCACCAAGCAATATCATTTATGCTGGCTGAAATGGCAATGAAAGTTGAACTAGCTAGAAT
GAGTTACCAGAGAGCAGCTTGGGAGGTTCATTCTGGTCGTCGAAATACCTATTATGCTTCTATTGCAA
AGGCATTTGCTGGAGATATTGCAAATCAGTTAGCTACTGATGCTGTGCAGATACTTGGAGGCAATGGA
TTTAATACAGAATATCCTGTAGAAAAACTAATGAGGGATGCCAAAATCTATCAGATTTATGAAGGTAC
TTCACAAATTCAAAGACTTATTGTAGCCCGTGAACACATTGACAAGTACAAAAATGTCGACGGC

| | | | |
|---|---|---|---|
| NOV20c, 276657466 | | | |
| Protein Sequence | SEQ ID NO: 330 | 429 aa | MW at 47401.7kD |

TRSPTMAAGFGRCCRVLRSISRFHWRSQHTKANRQREPGLGFSFEFTEQQKEFQATARKFAREEIIPV
AAEYDKTGEYPVPLIRRAWELGLMNTHIPENCGGLGLGTFDACLISEELAYGCTGVQTAIEGNSLGQM
PIIIAGNDQQKKKYLGRMTEEPLMCAYCVTEPGAGSDVAGIKTKAEKKGDEYIINGQKMWITNGGKAN
WYFLLARSDPDPKAPANKAFTGFIVEADTPGIQIGRKELNMGQRCSDTRGIVFEDVKVPKENVLIGDG
AGFKVAMGAFDKTRPVVAAGAVGLAQRALDEATKYALERKTFGKLLVEHQAISFMLAEMAMKVELARM
SYQRAAWEVDSGRRNTYYASIAKAFAGDIANQLATDAVQILGGNGFNTEYPVEKLMRDAKIYQIYEGT
SQIQRLIVAREHIDKYKNVDG

| | | |
|---|---|---|
| NOV20d,276657530 | SEQ ID NO: 331 | 1387 bp |
| DNA Sequence | ORF Start: at 2 | ORF Stop: end of sequence |

CACCAGATCTCCCACCATGGCAGCGGGGTTCGGGCGATGCTGCAGGGTCCTGAGAAGTATTTCTCGTT
TTCATTGGAGATCACAGCATACAAAAGCCAATCGACAACGTGAACCAGGATTAGGATTTAGTTTTGAG
TTCACCGAACAGCAGAAAGAATTTCAAGCTACTGCTCGTAAATTTGCCAGAGAGGAAATCATCCCAGT
GGCTGCAGAATATGATAAAACTGGTGAATATCCAGTCCCCCTAATTAGAAGAGCCTGGGAACTTGGTT
TAATGAACACACACATTCCAGAGAACTGTGACTACAGTGTTTGCCCACTTTTGGAAGCTTGCACTCTA
TACCTAGATGCGTTTTTCCTTCTTCTAACTGGTTCCAACCTTAACTTGCACCTAAACCTTGGAGGTCT
TGGACTTGGAACTTTTGATGCTTGTTTAATTAGTGAAGAATTGGCTTATGGATGTACAGGGGTTCAGA
CTGCTATTGAAGGAAATTCTTTGGGGCAAATGCCTATTATTATTGCTGGAAATGATCAACAAAAGAAG
AAGTATTTGGGGAGAATGACTGAGGAGCCATTGATGTGTGCTTATTGTGTAACAGAACCTGGAGCAGG
CTCTGATGTAGCTGGTATAAAGACCAAAGCAGAAAAGAAAGGAGATGAGTATATTATTAATGGTCAGA
AGATGTGGATAACCAACGGAGGAAAAGCTAATTGGTATTTTTTATTGGCACGTTCTGATCCAGATCCT
AAAGCTCCTGCTAATAAAGCCTTTACTGGATTCATTGTGGAAGCAGATACCCCAGGAATTCAGATTGG
GAGAAAGGAATTAAACATGGGCCAGCGATGTTCAGATACTAGAGGAATTGTCTTCGAAGATGTGAAAG
TGCCTAAAGAAAATGTTTTAATTGGTGACGGAGCTGGTTTCAAAGTTGCAATGGGAGCTTTTGATAAA
ACCAGACCTGTAGTAGCTGCTGGTGCTGTTGGATTAGCACAAAGAGCTTTGGATGAAGCTACCAAGTA
TGCCCTGGAAAGGAAAACTTTCGGAAAGCTACTTGTAGAGCACCAAGCAATATCATTTATGCTGGCTG
AAATGGCAATGAAAGTTGAACTAGCTAGAATGAGTTACCAGAGAGCAGCTTGGGAGGTTGATTCTGGT
CGTCGAAATACCTATTATGCTTCTATTGCAAAGGCATTTGCTGGAGATATTGCAAATCAGTTAGCTAC
TGATGCTGTGCAGATACTTGGAGGCAATGGATTTAATACAGAATATCCTGTAGAAAAACTAATGAGGG
ATGCCAAAATCTATCAGATTTATGAAGGTACTTCACAAATTCAAAGACTTATTGTAGCCCGTGAACAC
ATTGACAAGTACAAAAATGTCGACGGC

| | | | |
|---|---|---|---|
| NOV20d, 276657530 | | | |
| Protein Sequence | SEQ ID NO: 332 | 462 aa | MW at 51083.9kD |

TRSPTMAAGFGRCCRVLRSISRFHWRSQHTKANRQREPGLGFSFEFTEQQKEFQATARKFAREEIIPV
AAEYDKTGEYPVPLIRRAWELGLMNTHIPENCDYSVCPLLEACTLYLDAFFLLLTGSNLNLHLNLGGL
GLGTFDACLISEELAYGCTGVQTAIEGNSLGQMPIIIAGNDQQKKKYLGRMTEEPLMCAYCVTEPGAG
SDVAGIKTKAEKKGDEYIINGQKMWITNGGKANWYFLLARSDPDPKAPANKAFTGFIVEADTPGIQIG
RKELNMGQRCSDTRGIVFEDVKVPKENVLIGDGAGFKVAMGAFDKTRPVVAAGAVGLAQRALDEATKY
ALERKTFGKLLVEHQAISFMLAEMAMKVELARMSYQRAAWEVDSGRRNTYYASIAKAFAGDIANQLAT
DAVQILGGNGFNTEYPVEKLMRDAKIYQIYEGTSQIQRLIVAREHIDKYKNVDG

| | | |
|---|---|---|
| NOV20e, 276657538 | SEQ ID NO: 333 | 1300 bp |
| DNA Sequence | ORF Start: at 2 | ORF Stop: end of sequence |

CACCAGATCTCCCACCATGGCAGCGGGGTTCGGGCGATGCTGCAGGTGTTCTTTACAGGTCCTGAGAA
GTATTTCTCGTTTTCATTGGAGATCACAGCATACAAAAGCCAATCGACAACGTGAACCAGGATTAGGA
TTTAGTTTTGAGTTCACCGAACAGCAGAAAGAATTTCAAGCTACTGCTCGTAAATTTGCCAGAGAGGA
AATCATCCCAGTGGCTGCAGAATATGATAAAACTGGTGAATATCCAGTCCCCCTAATTAGAAGAGCCT
GGGAACTTGGTTTAATGAACACACACATTCCAGAGAACTGTGGAGGTCTTGGACTTGGAACTTTTGAT
GCTTGTTTAATTAGTGAAGAATTGGCTTATGGATGTACAGGGGTTCAGACTGCTATTGAAGGAAATTC
TTTGGGGCAAATGCCTATTATTATTGCTGGAAATGATCAACAAAAGAAGAAGTATTTGGGGAGAATGA
CTGAGGAGCCATTGATGTGTGCTTATTGTGTAACAGAACCTGGAGCAGGCTCTGATGTAGCTGGTATA
AAGACCAAAGCAGAAAAGAAAGGACATGAGTATATTATTAATGGTCAGAAGATGTGGATAACCAACGG
AGGAAAAGCTAATTGGTATTTTTATTGGCACGTTCTGATCCAGATCCTAAAGCTCCTGCTAATAAAG
CCTTTACTGGATTCATTGTGGAAGCAGATACCCCAGGAATTCAGATTGGGAAAAAGGAATTAAACATG
GGCCAGCGATGTTCACATACTAGAGGAATTGTCTTCGAAGATGTGAAGTGCCTAAAGAAAATGTTTT
AATTGGTGACGGAGCTGGTTTCAAAGTTGCAATGGGAGCTTTTGATAAAACCAGACCTGTAGTAGCTG
CTGGTGCTGTTGGATTAGCACAAAGAGCTTTGGATGAAGCTACCAAGTATGCCCTGGAAAGGAAAACT
TTCGGAAAGCTACTTGTAGAGCACCAAGCAATATCATTATGCTGGCTGAAATGGCAATGAAAGTTGA
ACTAGCTAGAATGAGTTACCAGAGAGCAGCTTGGGAGGTTGATTCTGGTCGTCGAAATACCTATTATG
CTTCTATTGCAAAGGCATTTGCTGGAGATATTGCAAATCAGTTAGCTACTGATGCTGTGCAGATACTT
GGAGGCAATGGATTTAATACAGAATATCCTGTAGAAAAACTAATGAGGGATGCCAAATCTATCAGAT
TTATGAAGGTACTTCACAAATTCAAAGACTTATTGTAGCCCGTGAACACATTGACAAGTACAAAAATG
TCGACGGC

| | | | |
|---|---|---|---|
| NOV20e, 276657538 | | | |
| Protein Sequence | SEQ ID NO: 334 | 433 aa | MW at 47805.2kD |

TRSPTMAAGFGRCCRCSLQVLRSISRFHWRSQHTKANRQREPGLGFSFEFTEQQKEFQATARKFAREE
IIPVAAEYDKTGEYPVPLIRRAWELGLMNTHIPENCGGLGLGTFDACLISEELAYGCTGVQTAIEGNS
LGQMPIIIAGNDQQKKKYLGRMTEEPLMCAYCVTEPGAGSDVAGIKTKAEKKGDEYIINGQKMWITNG
GKANWYFLLARSDPDPKAPANKAFTGFIVEADTPGIQIGKKELNMGQRCSDTRGIVFEDVKVPKENVL
IGDGAGFKVAMGAFDKTRPVVAAGAVGLAQRALDEATKYALERKTFGKLLVEHQAISFMLAEMAMKVE
LARMSYQRAAWEVDSGRRNTYYASIAKAFAGDIANQLATDAVQILGGNGFNTEYPVEKLMRDAKIYQI
YEGTSQIQRLIVAREHIDKYKNVDG

| | | |
|---|---|---|
| NOV20f, 276657616 | SEQ ID NO: 335 | 1147 bp |
| DNA Sequence | ORF Start: at 2 | ORF Stop: end of sequence |

CACC6AGATCTTTCACCGAACAGCAGAAAGAATTTCAAGCTACTGCTCGTAAATTTGCCAGAGAGGAAA
TCATCCCAGTGGCTGCAGAATATGATAAAACTGGTGAATATCCAGTCCCCCTAATTAGAAGAGCCTGG
GAACTTGGTTTAATGAACACACACATTCCAGAGAACTGTGGAGGTCTTGGACTTGGAACTTTTGATGC
TTGTTTAATTAGTGAAGAATTGGCTTATGGATGTACAGGGGTTCAGACTGCTATTGAAGGAAATTCTT
TGGGGCAAATGCCTATTATTATTGCTGGAAATGATCAACAAAAGAAGAAGTATTTGGGGAGAATGACT
GAGGAGCCATTGATGTGTGCTTATTGTGTAACAGAACCTGGAGCAGGCTCTGATGTAGCTGGTATAAA
GACCAAAGCAGAAAAGAAAGGAGATGAGTATATTATTAATGGTCAGAAGATGTGGATAACCAACGGAG
GAAAAGCTAATTGGTATTTTTTATTGGCACGTTCTGATCCAGATCCTAAAGCTCCTGCTAATAAAGCC
TTTACTGGATTCATTGTGGAAGCAGATACCCCAGGAATTCAGATTGGGAGAAAGGAATTAAACATGGG
CCAGCGATGTTCAGATACTAGAGGAATTGTCTTCGAAGATGTGAAAGTGCCTAAAGAAAATGTTTTAA
TTGGTGACGGAGCTGGTTTCAAAGTTGCAATGGGAGCTTTTGATAAAACCAGACCTGTAGTAGCTGCT
GGTGCTGTTGGATTAGCACAAAGAGCTTTGGATGAAGCTACCAAGTATGCCCTGGAAAGGAAAACTTT
CGGAAAGCTACTTGTAGAGCACCAAGCAATATCATTTATGCTGGCTGAAATGGCAATGAAAGTTGAAC
TAGCTAGAATGAGTTACCAGAGAGCAGCTTGGGAGGTTGATTCTGGTCGTCGAAATACCTATTATGCT
TCTATTGCAAAGGCATTTGCTGGAGATATTGCAAATCAGTTAGCTACTGATGCTGTGCAGATACTTGG
AGGCAATGGATTTAATACAGAATATCCTGTAGAAAAACTAATGAGGGATGCCAAAATCTATCAGATTT
ATGAAGGTACTTCACAAATTCAAAGACTTATTGTAGCCCGTGAACACATTGTCGACGGC

| | | | |
|---|---|---|---|
| NOV20f, 276657616 | | | |
| Protein Sequence | SEQ ID NO: 336 | 382 aa | MW at 41891.5kD |

TRSFTEQQKEFQATARKFAREEIIPVAAEYDKTGEYPVPLIRRAWELGLMNTHIPENCGGLGLGTFDA
CLISEELAYGCTGVQTAIEGNSLGQMPIIIAGNDQQKKKYLGRMTEEPLMCAYCVTEPGAGSDVAGIK
TKAEKKGDEYIINGQKMWITNGGKANWYFLLARSDPDPKAPANKAFTGFIVEADTPGIQIGRKELNMG
QRCSDTRGIVFEDVKVPKENVLIGDGAGFKVAMGAFDKTRPVAAGAVGLAQRALDEATKYALERKTF
GKLLVEHQAISFMLAEMAMKVELARMSYQRAAWEVDSGRRNTYYASIAKAFAGDIANQLATDAVQILG
GNGFNTEYPVEKLMRDAKIYQIYEGTSQIQRLIVAREHIVDG

| | | |
|---|---|---|
| NOV20g, CG96778-03 | SEQ ID NO: 337 | 1278 bp |
| DNA Sequence | ORF Start: ATG at 1 | ORF Stop: TAA at 1276 |

ATGGCAGCGGGGTTCGGGCGATGCTGCAGGTGTTCTTTACAGGTCCTGAGAAGTATTTCTCGTTTTCA
TTGGAGATCACAGCATACAAAAGCCAATCGACAACGTGAACCAGGATTAGGATTTAGTTTTGAGTTCA
CCGAACAGCAGAAAGAATTTCAAGCTACTGCTCGTAAATTTGCCAGAGAGGAAATCATCCCAGTGGCT
GCAGAATATGATAAAACTGGTGAATATCCAGTCCCCCTAATTAGAAGAGCCTGGGAACTTGGTTTAAT
GAACACACACATTCCAGAGAACTGTGGAGGTCTTGGACTTGGAACTTTTGATGCTTGTTTAATTAGTG
AAGAATTGGCTTATGGATGTACAGGGGTTCAGACTGCTATTGAAGGAAATTCTTTGGGGCAAATGCCT
ATTATTATTGCTGGAAATGATCAACAAAAGAAGAAGTATTTGGGGACAATGACTGAGGAGCCATTGAT
GTGTGCTTATTGTGTAACAGAACCTGGAGCAGGCTCTGATGTAGCTGGTATAAAGACCAAAGCAGAAA
AGAAAGGAGATGAGTATATTATTAATGGTCAGAAGATGTGGATAACCAACGGAGGAAAAGCTAATTGG
TATTTTTTATTGGCACGTTCTGATCCAGATCCTAAAGCTCCTGTAATAAAGCCTTTACTGGATTCAT
TGTGGAAGCAGATACCCCAGGAATTCAGATTGGGAAAAAGGAATTAAACATGGGCCAGCGATGTTCAG
ATACTAGAGGAATTGTCTTCGAAGATGTGAAAGTGCCTAAAGAAAATGTTTTAATTGGTGACGGAGCT
GGTTTCAAAGTTGCAATGGGAGCTTTTGATAAAACCAGACCTGTAGTAGCTGCTGGTGCTGTTGGATT
AGCACAAAGAGCTTTGGATGAAGCTACCAAGTATGCCCTGGAAAGGAAAACTTTCGGAAAGCTACTTG
TAGAGCACCAAGCAATATCATTTATGCTGGCTGAAATGGCAATGAAAGTTGAACTAGCTAGAATGAGT
TACCAGAGAGCAGCTTGGGAGGTTGATTCTGGTCGTCGAAATACCTATTATGCTTCTATTGCAAAGGC
ATTTGCTGGAGATATTGCAAATCAGTTAGCTACTGATGCTGTGCAGATACTTGGAGGCAATGGATTTA
ATACAGAATATCCTGTAGAAAAACTAATGAGGGATGCCAAAATCTATCAGATTTATGAAGGTACTTCA
CAAATTCAAAGACTTATTGTAGCCCGTGAACACATTGACAAGTACAAAAATTAA

| | | | |
|---|---|---|---|
| NOV20g, CG96778-03 | | | |
| Protein Sequence | SEQ ID NO: 338 | 425 aa | MW at 46991.4kD |

MAAGFGRCCRCSLQVLRSISRFHWRSQHTKANRQREPGLGFSFEFTEQQKEFQATARKAFAREEIIPVA
AEYDKTGEYPVPLIRRAWELGLMNTHIPENCGGLGLCTFDACLISEELAYGCTGVQTAIEGNSLGQMP
IIIAGNDQQKKKYLGRMTEEPLMCAYCVTEPGAGSDVAGIKTKAEKKGDEYIINGQKMWITNGGKANW
YFLLARSDPDPKAPANKAFTGFIVEADTPGIQIGKKELNMGQRCSDTRGIVFEDVKVPKENVLIGDGA
GFKVAMGAFDKTRPVVAAGAVGLAQRALDEATKYALERKTFGKLLVEHQAISFMLAEMAMKVELARMS
YQRAAWEVDSGRRNTYYASIAKAFAGDIANQLATDAVQILGGNGFNTEYPVEKLMRDAKIYQIYEGTS
QIQRLIVAREHIDKYKN

| | | |
|---|---|---|
| NOV20h, 13382351 SNP for | SEQ ID NO: 339 | |
| CG96778-01 | ORF Start: ATG at | 3387 bp SNP: 1673 G/T |
| DNA Sequence | 1387 | ORF Stop: TAA at 2650 |

CTGCAGGACAGACAAACAAGGGGTAGCTTGCTTGGGTGAATGGTGGCAGGAACTACCGACTAGACATGT
TTAAGATGAGGGCTCCATCTTCGCTTCTCTGCCAGCCACGTGTACAGTAAGAAGGGGTTACAATAGGCAT
ATGGGTGATTTTGTGCTTTTCGTTCATCTTTTCTGTGTTTAAAATGTTCAGAATAAGAAATTGGAACAAA
GGAGACATGAATAGACAATTCCTAATCATCTTTAAGAGTCCGCTTTGTGTTTTCATTACACACCACCACT
TTGAAGGCTTTCTTGCCTATACCAGGACAAACTCAGCTCTTTATCCCTTTTCCGAATTTTCCTGGTACTT
TCACTTTGAATATAGCGCTTAATTAACATTCTGCCTTGTACCTAGGACTAACACACTATAAATTCCCAGA
AGACAAAGTAGGGGAATACAATAACAGGATATAGAATTTTAACAGCTAAATTAGATGAATTTATGGGTGA
CCTTTATTGGGCAAAAGAAAATGTTAAGTTAGTATAAGATTTAGTATAAGCTACCACTCAAAACTCAGGG
TCTCACTGGAAGAGAAAGTGACTCCAGGTAGAATTCCTCAGGGAGACATTCACTTCCATCATTCGCTGAA
CCAGGAGCTTTGGACAGCCTCGGATTGCACCCGCATATCCAAGGACACCACATCAGCGGACAAGTCATAA
ACAGCCTTGGGAATACGCGGAAAGGTCAAATTTACCTAAACAATTAAATTCTCTTTAAATTTTAAGGAA
ACACAAGTATGCTTTCGCTTTAGGTAGGGCATTTGAGAGCAAAATGTACTAATACTTTGAATCCGCCAAG
CAGACACGATCTGGGTTTGACCTTTCTCTCCGGGTAAAGGTGAAGGCTGACCACGGGGCCGCTCTCCCTC
CAGCCCCAGCCACGCCCTCTAACCAGGTTCCCGTCCTGCACCGCGCGCAAGTCCCCCCACCGTTCAGC
GCAACCGGCCCTCCCAGCCCCGCCGCCGTCCCCCTCCCCGCCCTGGCTCTCTTTCCGCGCTGCGTCAGCC
TCGGCGTCCCACAGAGAGGGCCAGAGGTGGAAACGAGAAAACCAAACCAGGACTATCAGAGATTGCCCG
GAGAGGGGATGCGACCCCTCCCCAGGTCGCAGCGACGGCGCACGCAAGGGTCACGGACCATGCGTTGGCT
ATCCGGCGCCGGGGACCGCTGCCACCCCGCCTAGCGCAGCGCCCCGTCCTTCCGAGCCAACCGCCTCT
TCCCGCCCCGCCCCATCCCGCCCCACGGGCTCCAGTGGGCGGGACCAGAGGAGTCCCGCGTTCGGGGAGT
ATGTCAAGGCCGTGACCCGTGTATTATTGTCCAGTGGCCGGAACGGAGAGCCAACATGGCAGCGGGGTT
CGGGCGATGCTGCAGGGTCCTGAGAAGTATTTCTCGTTTTCATTGGAGATCACAGCATACAAAAGCCAAT
CGACAACGTGAACCAGGATTAGGATTTAGTTTTGAGTTCACCGAACAGCAGAAAGAATTTCAAGCTACTG
CTCGTAAATTTGCCAGAGAGGAAATCATCCCAGTGGCTGCAGAATATGATAAAACTGGTGAATATCCAGT
CCCCCTAATTAGAAGAGCCTGGGAACTTGGTTAATGAACACACACATTCCAGAGAACTGTGTAGGTCTT
GGACTTGGAACTTTTGATGCTTGTTTAATTAGTGAAGAATTGGCTTATGGATGTACAGGGGTTCAGACTG
CTATTGAAGGAAATTCTTTGGGGCAAATGCCTATTATTATTGCTGGAAATGATCAACAAAAGAAGAAGTA
TTTGGGGAGAATGACTGAGGAGCCATTGATGTGTGCTTATTGTGTAACAGAACCTGGAGCAGGCTCTGAT
GTAGCTGGTATAAAGACCAAAGCAGAAAAGAAAGGAGATGAGTATATTATTAATGGTCAGAAGATGTGGA
TAACCAACGGAGGAAAAGCTAATTGGTATTTTTTATTGGCACGTTCTGATCCAGATCCTAAAGCTCCTGC
TAATAAAGCCTTTACTGGATTCATTGTGGAAGCAGATACCCCAGGAATTCAGATTGGGAGAAAGGAATTA
AACATGGGCCAGCGATGTTCAGATACTAGAGGAATTGTCTTCGAAGATGTGAAAGTGCCTAAAGAAAATG
TTTTAATTGGTGACGGAGCTGGTTTCAAAGTTGCAATGGGAGCTTTGATAAAACCAGACCTGTAGTAGC
TGCTGGTGCTGTTGGATTAGCACAAAGAGCTTTGGATGAAGCTACCAAGTATGCCCTGGAAAGGAAAACT
TTCGGAAAGCTACTTGTAGAGCACCAAGCAATATCATTTATGCTGGCTGAAATGGCAATGAAAGTTGAAC
TAGCTAGAATGAGTTACCAGAGAGCAGCTTGGGAGGTTGATTCTGGTCGTCGAAATACCTATTATGCTTC
TATTGCAAAGGCATTTGCTGGAGATATTGCAAATCAGTTAGCTACTGATGCTGTGCAGATACTTGGAGGC
AATGGATTTAATACAGAATATCCTGTAGAAAAACTAATGAGGGATGCCAAAATCTATCAGATTTATGAAG
GTACTTCACAAATTCAAAGACTTATTGTAGCCCGTGAACACATTGACAAGTACAAAAATTAAAAAAATTA
CTGTAGAAATATTGAATAACTAGAACACAAGCCACTGTTTCAGCTCCAGAAAAAAGAAAGGGCTTTAACG
TTTTTTCCAGTGAAAACAAATCCTCTTATATTAAATCTAAGCAACTGCTTATTATAGTAGTTTATACTTT
TGCTTAACTCTGTTATGTCTCTTAAGCAGGTTTGGTTTTTATTAAAATGATGTGTTTTCTTTAGTACCAC
TTTACTTGAATTACATTAACCTAGAAAACTACATAGGTTATTTTGATCTCTTAAGATTAATGTAGCAGAA
ATTTCTTGGAATTTTATTTTTGTAATGACAGAAAAGTGGGCTTAGAAAGTATTCAAGATGTTACAAAATT
TACATTTAGAAAATATTGTAGTATTTGAATACTGTCAACTTGACAGTAACTTTGTAGACTTAATGGTATT
ATTAAAGTTCTTTTTATTGCAGTTTGGAAAGCATTTGTGAAACTTTCTGTTTGGCACAGAAACAGTCAAA
ATTTTGACATTCATATTCTCCTATTTTACAGCTACAAGAACTTTCTTGAAAATCTTATTTAATTCTGAGC
CCATATTTCACTTACCTTATTTAAAATAAATCAATAAAGCTTGCCTTAAATTATTTTTATATGACTGTTG
GTCTCTAGGTAGCCTTTGGTCTATTGTACACAATCTCATTTCATATGTTTGCATTTTGGCAAAGAACTTA
ATAAAATTGTTCAGTGCTTATTATCAT

| | | | |
|---|---|---|---|
| NOV20h, 13382351 SNP for | | | |
| CG96778-01 | SEQ ID NO: | | |
| Protein Sequence | 340 | 421 aa | SNP: Gly to Val at position 96 |

MAAGFGRCCRVLRSISRFHWRSQHTKANRQREPGLGFSFEFTEQQKEFQATARKFAREEIIPVAAEYDKT
GEYPVPLIRRAWELGLMNTHIPENCVGLGLGTFDACLISEELAYGCTGVQTAIEGNSLGQMPIIIAGNDQ
QKKKYLGRMTEEPLMCAYCVTEPGAGSDVAGIKTKAEKKGDEYIINGQKMWITNGGKANWYFLLARSDPD
PKAPANKAFTGFIVEADTPGIQIGRKELNMGQRCSDTRGIVFEDVKVPKENVLIGDGAGFKVAMGAFDRT
RPVVAAGAVGLAQRALDEATKYALERKTFGKLLVEHQAISFMLAEMAMKVELARMSYQRAAWEVDSGRRN
TYYASIAKAFAGDIANQLATDAVQILGGNGFNTEYPVEKLMRDAKIYQIYEGTSQIQRLIVAREHIDKYK
N

| | | |
|---|---|---|
| | | 3387 bp SNP: 1717 |
| NOV20i, 13382352 SNP for | SEQ ID NO: 341 | G/C |
| CG96778-01 | ORF Start: ATG at | ORF Stop: TAA at |
| DNA Sequence | 1387 | 2650 |

CTGCAGGACAGACAAACAAGGGGTAGCTTGCTTGGGTGAATGGTGGCAGGAACTACCGACTAGACATGT
TTAAGATGAGGGCTCCATCTTCGCTTCTCTGCCAGCCACGTGTACAGTAAGAAGGGGTTACAATAGGCAT
ATGGGTGATTTTGTGCTTTTCGTTCATCTTTTCTGTGTTTAAAATGTTCAGAATAAGAAATTGGAACAAA
GGAGACATGAATAGACAATTCCTAATCATCTTTAAGAGTCCGCTTTGTGTTTTCATTACACACCACCACT
TTGAAGGCTTTCTTGCCTATACCAGGACAAACTCAGCTCTTTATCCCTTTTCCGAATTTTCCTGGTACTT
TCACTTTGAATATAGCGCTTAATTAACATTCTGCCTTGTACCTAGGACTAACACACTATAAATTCCCAGA
AGACAAAGTAGGGGAATACAATAACAGGATATAGAATTTTAACAGCTAAATTAGATGAATTTATGGGTGA
CCTTTATTGGGCAAAAGAAAATGTTAAGTTAGTATAAGATTTAGTATAAGCTACCACTCAAAACTCAGGG
TCTCACTGGAAGAGAAAGTGACTCCAGGTAGAATTCCTCAGGGAGACATTCACTTCCATCATTCGCTGAA
CCAGGAGCTTTGGACAGCCTCGGATTGCACCCGCATATCCAAGGACACCACATCAGCGGACAAGTCATAA
ACAGCCTTGGGAATACGCGGAAAGGTCAAATTTACCTAAACAATTAAATTCTCTTTAAATTTTAAGGAA
ACACAAGTATGCTTTCGCTTTAGGTAGGGCATTTGAGAGCAAAATGTACTAATACTTTGAATCCGCCAAG
CAGACACGATCTGGGTTTGACCTTTCTCTCCGGGTAAAGGTGAAGGCTGACCACGGGGCCGCTCTCCCTC
CAGCCCCAGCCACGCCCTCTAACCAGGTTCCCGTCCTGCACCGCGCGCAAGTCCCCCCACCGTTCAGC
GCAACCGGCCCTCCCAGCCCCGCCGCCGTCCCCCTCCCCGCCCTGGCTCTCTTTCCGCGCTGCGTCAGCC
TCGGCGTCCCACAGAGAGGGCCAGAGGTGGAAACGAGAAAACCAAACCAGGACTATCAGAGATTGCCCG
GAGAGGGGATGCGACCCCTCCCCAGGTCGCAGCGACGGCGCACGCAAGGGTCACGGACCATGCGTTGGCT
ATCCGGCGCCGGGGACCGCTGCCACCCCGCCTAGCGCAGCGCCCCGTCCTTCCGAGCCAACCGCCTCT
TCCCGCCCCGCCCCATCCCGCCCCACGGGCTCCAGTGGGCGGGACCAGAGGAGTCCCGCGTTCGGGGAGT
ATGTCAAGGCCGTGACCCGTGTATTATTGTCCAGTGGCCGGAACGGAGAGCCAACATGGCAGCGGGGTT
CGGGCGATGCTGCAGGGTCCTGAGAAGTATTTCTCGTTTTCATTGGAGATCACAGCATACAAAAGCCAAT
CGACAACGTGAACCAGGATTAGGATTTAGTTTTGAGTTCACCGAACAGCAGAAAGAATTTCAAGCTACTG
CTCGTAAATTTGCCAGAGAGGAAATCATCCCAGTGGCTGCAGAATATGATAAAACTGGTGAATATCCAGT
CCCCCTAATTAGAAGAGCCTGGGAACTTGGTTAATGAACACACACATTCCAGAGAACTGTGTAGGTCTT
GGACTTGGAACTTTTGATGCTTGTTTAATTAGTGAAGAATTGGCTTATGGATGTACAGGGGTTCAGACTG
CTATTGAAGGAAATTCTTTGGGGCAAATGCCTATTATTATTGCTGGAAATGATCAACAAAAGAAGAAGTA
TTTGGGGAGAATGACTGAGGAGCCATTGATGTGTGCTTATTGTGTAACAGAACCTGGAGCAGGCTCTGAT
GTAGCTGGTATAAAGACCAAAGCAGAAAAGAAAGGAGATGAGTATATTATTAATGGTCAGAAGATGTGGA
TAACCAACGGAGGAAAAGCTAATTGGTATTTTTTATTGGCACGTTCTGATCCAGATCCTAAAGCTCCTGC
TAATAAAGCCTTTACTGGATTCATTGTGGAAGCAGATACCCCAGGAATTCAGATTGGGAGAAAGGAATTA
AACATGGGCCAGCGATGTTCAGATACTAGAGGAATTGTCTTCGAAGATGTGAAAGTGCCTAAAGAAAATG
TTTTAATTGGTGACGGAGCTGGTTTCAAAGTTGCAATGGGAGCTTTGATAAAACCAGACCTGTAGTAGC
TGCTGGTGCTGTTGGATTAGCACAAAGAGCTTTGGATGAAGCTACCAAGTATGCCCTGGAAAGGAAAACT
TTCGGAAAGCTACTTGTAGAGCACCAAGCAATATCATTTATGCTGGCTGAAATGGCAATGAAAGTTGAAC
TAGCTAGAATGAGTTACCAGAGAGCAGCTTGGGAGGTTGATTCTGGTCGTCGAAATACCTATTATGCTTC
TATTGCAAAGGCATTTGCTGGAGATATTGCAAATCAGTTAGCTACTGATGCTGTGCAGATACTTGGAGGC
AATGGATTTAATACAGAATATCCTGTAGAAAAACTAATGAGGGATGCCAAAATCTATCAGATTTATGAAG
GTACTTCACAAATTCAAAGACTTATTGTAGCCCGTGAACACATTGACAAGTACAAAAATTAAAAAAATTA
CTGTAGAAATATTGAATAACTAGAACACAAGCCACTGTTTCAGCTCCAGAAAAAAGAAAGGGCTTTAACG
TTTTTTCCAGTGAAAACAAATCCTCTTATATTAAATCTAAGCAACTGCTTATTATAGTAGTTTATACTTT
TGCTTAACTCTGTTATGTCTCTTAAGCAGGTTTGGTTTTTATTAAAATGATGTGTTTTCTTTAGTACCAC
TTTACTTGAATTACATTAACCTAGAAAACTACATAGGTTATTTTGATCTCTTAAGATTAATGTAGCAGAA
ATTTCTTGGAATTTTATTTTTGTAATGACAGAAAAGTGGGCTTAGAAAGTATTCAAGATGTTACAAAATT
TACATTTAGAAAATATTGTAGTATTTGAATACTGTCAACTTGACAGTAACTTTGTAGACTTAATGGTATT
ATTAAAGTTCTTTTTATTGCAGTTTGGAAAGCATTTGTGAAACTTTCTGTTTGGCACAGAAACAGTCAAA
ATTTTGACATTCATATTCTCCTATTTTACAGCTACAAGAACTTTCTTGAAAATCTTATTTAATTCTGAGC
CCATATTTCACTTACCTTATTTAAAATAAATCAATAAAGCTTGCCTTAAATTATTTTTATATGACTGTTG
GTCTCTAGGTAGCCTTTGGTCTATTGTACACAATCTCATTTCATATGTTTGCATTTTGGCAAAGAACTTA
ATAAAATTGTTCAGTGCTTATTATCAT

| | | | |
|---|---|---|---|
| NOV20i, 13382352 SNP for | | | |
| CG96778-01 | SEQ ID NO: | SNP: Glu to Gln at position | |
| Protein Sequence | 342 | 421 aa | 111 |

MAAGFGRCCRVLRSISRFHWRSQHTKANRQREPGLGFSFEFTEQQKEFQATARKFAREEIIPVAAEYDKT
GEYPVPLIRRAWELGLMNTHIPENCGGLGLGTFDACLISEQLAYGCTGVQTAIEGNSLGQMPIIIAGNDQ
QKKKYLGRMTEEPLMCAYCVTEPGAGSDVAGIKTKAEKKGDEYIINGQKMWITNGGKANWYFLLARSDPD
PKAPANKAFTGFIVEADTPGIQIGRKELNMGQRCSDTRGIVFEDVKVPKENVLIGDGAGFKVAMGAFDRT
RPVVAAGAVGLAQRALDEATKYALERKTFGKLLVEHQAISFMLAEMAMKVELARMSYQRAAWEVDSGRRN
TYYASIAKAFAGDIANQLATDAVQILGGNGFNTEYPVEKLMRDAKIYQIYEGTSQIQRLIVAREHIDKYK
N

| | | |
|---|---|---|
| Nov20j, 13382353 SNP for | | |
| CG96778-01 | SEQ ID NO: 343 | 3387 bp SNP: 2204 C/T |
| DNA Sequence | ORF Start: ATG at 1387 | ORF Stop: TAA at 2650 |

CTGCAGGACAGACAAACAAGGGGTAGCTTGCTTGGGTGAATGGTGGCAGGAACTACCGACTAGACATGT
TTAAGATGAGGGCTCCATCTTCGCTTCTCTGCCAGCCACGTGTACAGTAAGAAGGGGTTACAATAGGCAT
ATGGGTGATTTTGTGCTTTTCGTTCATCTTTTCTGTGTTTAAAATGTTCAGAATAAGAAATTGGAACAAA
GGAGACATGAATAGACAATTCCTAATCATCTTTAAGAGTCCGCTTTGTGTTTTCATTACACACCACCACT
TTGAAGGCTTTCTTGCCTATACCAGGACAAACTCAGCTCTTTATCCCTTTTCCGAATTTTCCTGGTACTT
TCACTTTGAATATAGCGCTTAATTAACATTCTGCCTTGTACCTAGGACTAACACACTATAAATTCCCAGA
AGACAAAGTAGGGGAATACAATAACAGGATATAGAATTTTAACAGCTAAATTAGATGAATTTATGGGTGA
CCTTTATTGGGCAAAAGAAAATGTTAAGTTAGTATAAGATTTAGTATAAGCTACCACTCAAAACTCAGGG
TCTCACTGGAAGAGAAAGTGACTCCAGGTAGAATTCCTCAGGGAGACATTCACTTCCATCATTCGCTGAA
CCAGGAGCTTTGGACAGCCTCGGATTGCACCCGCATATCCAAGGACACCACATCAGCGGACAAGTCATAA
ACAGCCTTGGGAATACGCGGAAAGGTCAAATTTACCTAAACAATTAAATTCTCTTTAAATTTTAAGGAA
ACACAAGTATGCTTTCGCTTTAGGTAGGGCATTTGAGAGCAAAATGTACTAATACTTTGAATCCGCCAAG
CAGACACGATCTGGGTTTGACCTTTCTCTCCGGGTAAAGGTGAAGGCTGACCACGGGGCCGCTCTCCCTC
CAGCCCCAGCCACGCCCTCTAACCAGGTTCCCGTCCTGCACCGCGCGCAAGTCCCCCCACCGTTCAGC
GCAACCGGCCCTCCCAGCCCCGCCGCCGTCCCCCTCCCCGCCCTGGCTCTCTTTCCGCGCTGCGTCAGCC
TCGGCGTCCCACAGAGAGGGCCAGAGGTGGAAACGAGAAAACCAAACCAGGACTATCAGAGATTGCCCG
GAGAGGGGATGCGACCCCTCCCCAGGTCGCAGCGACGGCGCACGCAAGGGTCACGGACCATGCGTTGGCT
ATCCGGCGCCGGGGACCGCTGCCACCCCGCCTAGCGCAGCGCCCCGTCCTTCCGAGCCAACCGCCTCT
TCCCGCCCCGCCCCATCCCGCCCCACGGGCTCCAGTGGGCGGGACCAGAGGAGTCCCGCGTTCGGGGAGT
ATGTCAAGGCCGTGACCCGTGTATTATTGTCCAGTGGCCGGAACGGAGAGCCAACATGGCAGCGGGGTT
CGGGCGATGCTGCAGGGTCCTGAGAAGTATTTCTCGTTTTCATTGGAGATCACAGCATACAAAAGCCAAT
CGACAACGTGAACCAGGATTAGGATTTAGTTTTGAGTTCACCGAACAGCAGAAAGAATTTCAAGCTACTG
CTCGTAAATTTGCCAGAGAGGAAATCATCCCAGTGGCTGCAGAATATGATAAAACTGGTGAATATCCAGT
CCCCCTAATTAGAAGAGCCTGGGAACTTGGTTAATGAACACACACATTCCAGAGAACTGTGTAGGTCTT
GGACTTGGAACTTTTGATGCTTGTTTAATTAGTGAAGAATTGGCTTATGGATGTACAGGGGTTCAGACTG
CTATTGAAGGAAATTCTTTGGGGCAAATGCCTATTATTATTGCTGGAAATGATCAACAAAAGAAGAAGTA
TTTGGGGAGAATGACTGAGGAGCCATTGATGTGTGCTTATTGTGTAACAGAACCTGGAGCAGGCTCTGAT
GTAGCTGGTATAAAGACCAAAGCAGAAAAGAAAGGAGATGAGTATATTATTAATGGTCAGAAGATGTGGA
TAACCAACGGAGGAAAAGCTAATTGGTATTTTTTATTGGCACGTTCTGATCCAGATCCTAAAGCTCCTGC
TAATAAAGCCTTTACTGGATTCATTGTGGAAGCAGATACCCCAGGAATTCAGATTGGGAGAAAGGAATTA
AACATGGGCCAGCGATGTTCAGATACTAGAGGAATTGTCTTCGAAGATGTGAAAGTGCCTAAAGAAAATG
TTTTAATTGGTGACGGAGCTGGTTTCAAAGTTGCAATGGGAGCTTTGATAAAACCAGACCTGTAGTAGC
TGCTGGTGCTGTTGGATTAGCACAAAGAGCTTTGGATGAAGCTACCAAGTATGCCCTGGAAAGGAAAACT
TTCGGAAAGCTACTTGTAGAGCACCAAGCAATATCATTTATGCTGGCTGAAATGGCAATGAAAGTTGAAC
TAGCTAGAATGAGTTACCAGAGAGCAGCTTGGGAGGTTGATTCTGGTCGTCGAAATACCTATTATGCTTC
TATTGCAAAGGCATTTGCTGGAGATATTGCAAATCAGTTAGCTACTGATGCTGTGCAGATACTTGGAGGC
AATGGATTTAATACAGAATATCCTGTAGAAAAACTAATGAGGGATGCCAAAATCTATCAGATTTATGAAG
GTACTTCACAAATTCAAAGACTTATTGTAGCCCGTGAACACATTGACAAGTACAAAAATTAAAAAAATTA
CTGTAGAAATATTGAATAACTAGAACACAAGCCACTGTTTCAGCTCCAGAAAAAAGAAAGGGCTTTAACG
TTTTTTCCAGTGAAAACAAATCCTCTTATATTAAATCTAAGCAACTGCTTATTATAGTAGTTTATACTTT
TGCTTAACTCTGTTATGTCTCTTAAGCAGGTTTGGTTTTTATTAAAATGATGTGTTTTCTTTAGTACCAC
TTTACTTGAATTACATTAACCTAGAAAACTACATAGGTTATTTTGATCTCTTAAGATTAATGTAGCAGAA
ATTTCTTGGAATTTTATTTTTGTAATGACAGAAAAGTGGGCTTAGAAAGTATTCAAGATGTTACAAAATT
TACATTTAGAAAATATTGTAGTATTTGAATACTGTCAACTTGACAGTAACTTTGTAGACTTAATGGTATT
ATTAAAGTTCTTTTTATTGCAGTTTGGAAAGCATTTGTGAAACTTTCTGTTTGGCACAGAAACAGTCAAA
ATTTTGACATTCATATTCTCCTATTTTACAGCTACAAGAACTTTCTTGAAAATCTTATTTAATTCTGAGC
CCATATTTCACTTACCTTATTTAAAATAAATCAATAAAGCTTGCCTTAAATTATTTTTATATGACTGTTG
GTCTCTAGGTAGCCTTTGGTCTATTGTACACAATCTCATTTCATATGTTTGCATTTTGGCAAAGAACTTA
ATAAAATTGTTCAGTGCTTATTATCAT

| | | | |
|---|---|---|---|
| NOV20j, 13382353 SNP for | | | |
| CG96778-01 | | | |
| Protein Sequence | SEQ ID NO: 344 | 421 aa | SNP: Ala to Val at 273 |

MAAGFGRCCRVLRSISRFHWRSQHTKANRQREPGLGFSFEFTEQQKEFQATARKFAREEIIPVAAEYDKT
GEYPVPLIRRAWELGLMNTHIPENCGGLGLGTFDACLISEELAYGCTGVQTAIEGNSLGQMPIIIAGNDQ
QKKKYLGRMTEEPLMACYCVTEPGAGSDVAGIKTKAEKKGDEYIINGQKMWITNGGKANWYFLLARSDPD
PKAPANKAFTGFIVEADTPGIQIGRKELNMGQRCSDTRGIVFEDVKVPKENVLIGDGAGFKVVMGAFDKT
RPVVAAGAVGLAQRALDEATKYALERKTFGKLLVEHQAISFMLAEMAMKVELARMSYQRAAWEVDSGRRN
TYYASIAKAFAGDIANQLATDAVQILGGNGFNTEYPVEKLMRDAKIYQIYEGTSQIQRLIVAREHIDKYK
N

| | | |
|---|---|---|
| NOV20k, 13382354 SNP for | | |
| CG96778-01 | SEQ ID NO: 345 | 3387 bp SNP: 2547 A/G |
| DNA Sequence | ORF Start: ATG at 1387 | ORF Stop: TAA at 2650 |

CTGCAGGACAGACAAACAAGGGGTAGCTTGCTTGGGTGAATGGTGGCAGGAACTACCGACTAGACATGT
TTAAGATGAGGGCTCCATCTTCGCTTCTCTGCCAGCCACGTGTACAGTAAGAAGGGGTTACAATAGGCAT
ATGGGTGATTTTGTGCTTTTCGTTCATCTTTTCTGTGTTTAAAATGTTCAGAATAAGAAATTGGAACAAA
GGAGACATGAATAGACAATTCCTAATCATCTTTAAGAGTCCGCTTTGTGTTTTCATTACACACCACCACT
TTGAAGGCTTTCTTGCCTATACCAGGACAAACTCAGCTCTTTATCCCTTTTCCGAATTTTCCTGGTACTT
TCACTTTGAATATAGCGCTTAATTAACATTCTGCCTTGTACCTAGGACTAACACACTATAAATTCCCAGA
AGACAAAGTAGGGGAATACAATAACAGGATATAGAATTTTAACAGCTAAATTAGATGAATTTATGGGTGA
CCTTTATTGGGCAAAAGAAAATGTTAAGTTAGTATAAGATTTAGTATAAGCTACCACTCAAAACTCAGGG
TCTCACTGGAAGAGAAAGTGACTCCAGGTAGAATTCCTCAGGGAGACATTCACTTCCATCATTCGCTGAA
CCAGGAGCTTTGGACAGCCTCGGATTGCACCCGCATATCCAAGGACACCACATCAGCGGACAAGTCATAA
ACAGCCTTGGGAATACGCGGAAAGGTCAAATTTACCTAAACAATTAAATTCTCTTTAAATTTTAAGGAA
ACACAAGTATGCTTTCGCTTTAGGTAGGGCATTTGAGAGCAAAATGTACTAATACTTTGAATCCGCCAAG
CAGACACGATCTGGGTTTGACCTTTCTCTCCGGGTAAAGGTGAAGGCTGACCACGGGGCCGCTCTCCCTC
CAGCCCCAGCCACGCCCTCTAACCAGGTTCCCGTCCTGCACCGCGCGCAAGTCCCCCCACCGTTCAGC
GCAACCGGCCCTCCCAGCCCCGCCGCCGTCCCCCTCCCCGCCCTGGCTCTCTTTCCGCGCTGCGTCAGCC
TCGGCGTCCCACAGAGAGGGCCAGAGGTGGAAACGAGAAAACCAAACCAGGACTATCAGAGATTGCCCG
GAGAGGGGATGCGACCCCTCCCCAGGTCGCAGCGACGGCGCACGCAAGGGTCACGGACCATGCGTTGGCT
ATCCGGCGCCGGGGACCGCTGCCACCCCGCCTAGCGCAGCGCCCCGTCCTTCCGAGCCAACCGCCTCT
TCCCGCCCCGCCCCATCCCGCCCCACGGGCTCCAGTGGGCGGGACCAGAGGAGTCCCGCGTTCGGGGAGT
ATGTCAAGGCCGTGACCCGTGTATTATTGTCCAGTGGCCGGAACGGAGAGCCAACATGGCAGCGGGGTT
CGGGCGATGCTGCAGGGTCCTGAGAAGTATTTCTCGTTTTCATTGGAGATCACAGCATACAAAAGCCAAT
CGACAACGTGAACCAGGATTAGGATTTAGTTTTGAGTTCACCGAACAGCAGAAAGAATTTCAAGCTACTG
CTCGTAAATTTGCCAGAGAGGAAATCATCCCAGTGGCTGCAGAATATGATAAAACTGGTGAATATCCAGT
CCCCCTAATTAGAAGAGCCTGGGAACTTGGTTAATGAACACACACATTCCAGAGAACTGTGTAGGTCTT
GGACTTGGAACTTTTGATGCTTGTTTAATTAGTGAAGAATTGGCTTATGGATGTACAGGGGTTCAGACTG
CTATTGAAGGAAATTCTTTGGGGCAAATGCCTATTATTATTGCTGGAAATGATCAACAAAAGAAGAAGTA
TTTGGGGAGAATGACTGAGGAGCCATTGATGTGTGCTTATTGTGTAACAGAACCTGGAGCAGGCTCTGAT
GTAGCTGGTATAAAGACCAAAGCAGAAAAGAAAGGAGATGAGTATATTATTAATGGTCAGAAGATGTGGA
TAACCAACGGAGGAAAAGCTAATTGGTATTTTTTATTGGCACGTTCTGATCCAGATCCTAAAGCTCCTGC
TAATAAAGCCTTTACTGGATTCATTGTGGAAGCAGATACCCCAGGAATTCAGATTGGGAGAAAGGAATTA
AACATGGGCCAGCGATGTTCAGATACTAGAGGAATTGTCTTCGAAGATGTGAAAGTGCCTAAAGAAAATG
TTTTAATTGGTGACGGAGCTGGTTTCAAAGTTGCAATGGGAGCTTTGATAAAACCAGACCTGTAGTAGC
TGCTGGTGCTGTTGGATTAGCACAAAGAGCTTTGGATGAAGCTACCAAGTATGCCCTGGAAAGGAAAACT
TTCGGAAAGCTACTTGTAGAGCACCAAGCAATATCATTTATGCTGGCTGAAATGGCAATGAAAGTTGAAC
TAGCTAGAATGAGTTACCAGAGAGCAGCTTGGGAGGTTGATTCTGGTCGTCGAAATACCTATTATGCTTC
TATTGCAAAGGCATTTGCTGGAGATATTGCAAATCAGTTAGCTACTGATGCTGTGCAGATACTTGGAGGC
AATGGATTTAATACAGAATATCCTGTAGAAAAACTAATGAGGGATGCCAAAATCTATCAGATTTATGAAG
GTACTTCACAAATTCAAAGACTTATTGTAGCCCGTGAACACATTGACAAGTACAAAAATTAAAAAAATTA
CTGTAGAAATATTGAATAACTAGAACACAAGCCACTGTTTCAGCTCCAGAAAAAAGAAAGGGCTTTAACG
TTTTTTCCAGTGAAAACAAATCCTCTTATATTAAATCTAAGCAACTGCTTATTATAGTAGTTTATACTTT
TGCTTAACTCTGTTATGTCTCTTAAGCAGGTTTGGTTTTTATTAAAATGATGTGTTTTCTTTAGTACCAC
TTTACTTGAATTACATTAACCTAGAAAACTACATAGGTTATTTTGATCTCTTAAGATTAATGTAGCAGAA
ATTTCTTGGAATTTTATTTTTGTAATGACAGAAAAGTGGGCTTAGAAAGTATTCAAGATGTTACAAAATT
TACATTTAGAAAATATTGTAGTATTTGAATACTGTCAACTTGACAGTAACTTTGTAGACTTAATGGTATT
ATTAAAGTTCTTTTTATTGCAGTTTGGAAAGCATTTGTGAAACTTTCTGTTTGGCACAGAAACAGTCAAA
ATTTTGACATTCATATTCTCCTATTTTACAGCTACAAGAACTTTCTTGAAAATCTTATTTAATTCTGAGC
CCATATTTCACTTACCTTATTTAAAATAAATCAATAAAGCTTGCCTTAAATTATTTTTATATGACTGTTG
GTCTCTAGGTAGCCTTTGGTCTATTGTACACAATCTCATTTCATATGTTTGCATTTTGGCAAAGAACTTA
ATAAAATTGTTCAGTGCTTATTATCAT

| | | | |
|---|---|---|---|
| NOV20k, 13382354 SNP | | | |
| for | | | |
| CG96778-01 | SEQ ID NO: | | SNP: no change in the protein |
| Protein Sequence | 346 | 421 aa | sequence |

MAAGFGRCCRVLRSISRFHWRSQHTKANRQREPGLGFSFEFTEQQKEFQATARKFAREEIIPVAAEYDKT
GEYPVPLIRRAWELGLMNTHIPENCGGLGLGTFDACLISEELAYGCTGVQTAIEGNSLGQMPIIIAGNDQ
QKKKYLGRMTEEPLMCAYCVTEPGAGSDVAGIKTKAEKKGDEYIINGQKMWITNGGKANWYFLLARSDPD
PKAPANKAFTGFIVEADTPGIQIGRKELNMGQRCSDTRGIVFEDVKVPKENVLIGDGAGFKVAMGAFDKT
RPVVAAGAVGLAQRALDEATKYALERKTFGKLLVEHQAISFMLAEMAMKVELARMSYQRAAWEVDSGRRN
TYYASIAKAFAFGDIANQLATDAVQILGGNGFNTEYPVEKLMRDAKIYQIYEGTSQIQRLIVAREHIDKYK
N

| | | |
|---|---|---|
| | | 3387 bp SNP: 3324 |
| NOV20l, 12252113 SNP for | SEQ ID NO: 347 | T/C |
| CG96778-01 | ORF Start: ATG at | ORF Stop: TAA at |
| DNA Sequence | 1387 | 2650 |

CTGCAGGACAGACAAACAAGGGGTAGCTTGCTTGGGTGAATGGTGGCAGGAACTACCGACTAGACATGT
TTAAGATGAGGGCTCCATCTTCGCTTCTCTGCCAGCCACGTGTACAGTAAGAAGGGGTTACAATAGGCAT
ATGGGTGATTTTGTGCTTTTCGTTCATCTTTTCTGTGTTTAAAATGTTCAGAATAAGAAATTGGAACAAA
GGAGACATGAATAGACAATTCCTAATCATCTTTAAGAGTCCGCTTTGTGTTTTCATTACACACCACCACT
TTGAAGGCTTTCTTGCCTATACCAGGACAAACTCAGCTCTTTATCCCTTTTCCGAATTTTCCTGGTACTT
TCACTTTGAATATAGCGCTTAATTAACATTCTGCCTTGTACCTAGGACTAACACACTATAAATTCCCAGA
AGACAAAGTAGGGGAATACAATAACAGGATATAGAATTTTAACAGCTAAATTAGATGAATTTATGGGTGA
CCTTTATTGGGCAAAAGAAAATGTTAAGTTAGTATAAGATTTAGTATAAGCTACCACTCAAAACTCAGGG
TCTCACTGGAAGAGAAAGTGACTCCAGGTAGAATTCCTCAGGGAGACATTCACTTCCATCATTCGCTGAA
CCAGGAGCTTTGGACAGCCTCGGATTGCACCCGCATATCCAAGGACACCACATCAGCGGACAAGTCATAA
ACAGCCTTGGGAATACGCGGAAAGGTCAAATTTACCTAAACAATTAAATTCTCTTTAAATTTTAAGGAA
ACACAAGTATGCTTTCGCTTTAGGTAGGGCATTTGAGAGCAAAATGTACTAATACTTTGAATCCGCCAAG
CAGACACGATCTGGGTTTGACCTTTCTCTCCGGGTAAAGGTGAAGGCTGACCACGGGGCCGCTCTCCCTC
CAGCCCCAGCCACGCCCTCTAACCAGGTTCCCGTCCTGCACCGCGCGCAAGTCCCCCCACCGTTCAGC
GCAACCGGCCCTCCCAGCCCCGCCGCCGTCCCCCTCCCCGCCCTGGCTCTCTTTCCGCGCTGCGTCAGCC
TCGGCGTCCCACAGAGAGGGCCAGAGGTGGAAACGAGAAAACCAAACCAGGACTATCAGAGATTGCCCG
GAGAGGGGATGCGACCCCTCCCCAGGTCGCAGCGACGGCGCACGCAAGGGTCACGGACCATGCGTTGGCT
ATCCGGCGCCGGGGACCGCTGCCACCCCGCCTAGCGCAGCGCCCCGTCCTTCCGAGCCAACCGCCTCT
TCCCGCCCCGCCCCATCCCGCCCCACGGGCTCCAGTGGGCGGGACCAGAGGAGTCCCGCGTTCGGGGAGT
ATGTCAAGGCCGTGACCCGTGTATTATTGTCCAGTGGCCGGAACGGAGAGCCAACATGGCAGCGGGGTT
CGGGCGATGCTGCAGGGTCCTGAGAAGTATTTCTCGTTTTCATTGGAGATCACAGCATACAAAAGCCAAT
CGACAACGTGAACCAGGATTAGGATTTAGTTTTGAGTTCACCGAACAGCAGAAAGAATTTCAAGCTACTG
CTCGTAAATTTGCCAGAGAGGAAATCATCCCAGTGGCTGCAGAATATGATAAAACTGGTGAATATCCAGT
CCCCCTAATTAGAAGAGCCTGGGAACTTGGTTAATGAACACACACATTCCAGAGAACTGTGTAGGTCTT
GGACTTGGAACTTTTGATGCTTGTTTAATTAGTGAAGAATTGGCTTATGGATGTACAGGGGTTCAGACTG
CTATTGAAGGAAATTCTTTGGGGCAAATGCCTATTATTATTGCTGGAAATGATCAACAAAAGAAGAAGTA
TTTGGGGAGAATGACTGAGGAGCCATTGATGTGTGCTTATTGTGTAACAGAACCTGGAGCAGGCTCTGAT
GTAGCTGGTATAAAGACCAAAGCAGAAAAGAAAGGAGATGAGTATATTATTAATGGTCAGAAGATGTGGA
TAACCAACGGAGGAAAAGCTAATTGGTATTTTTTATTGGCACGTTCTGATCCAGATCCTAAAGCTCCTGC
TAATAAAGCCTTTACTGGATTCATTGTGGAAGCAGATACCCCAGGAATTCAGATTGGGAGAAAGGAATTA
AACATGGGCCAGCGATGTTCAGATACTAGAGGAATTGTCTTCGAAGATGTGAAAGTGCCTAAAGAAAATG
TTTTAATTGGTGACGGAGCTGGTTTCAAAGTTGCAATGGGAGCTTTGATAAAACCAGACCTGTAGTAGC
TGCTGGTGCTGTTGGATTAGCACAAAGAGCTTTGGATGAAGCTACCAAGTATGCCCTGGAAAGGAAAACT
TTCGGAAAGCTACTTGTAGAGCACCAAGCAATATCATTTATGCTGGCTGAAATGGCAATGAAAGTTGAAC
TAGCTAGAATGAGTTACCAGAGAGCAGCTTGGGAGGTTGATTCTGGTCGTCGAAATACCTATTATGCTTC
TATTGCAAAGGCATTTGCTGGAGATATTGCAAATCAGTTAGCTACTGATGCTGTGCAGATACTTGGAGGC
AATGGATTTAATACAGAATATCCTGTAGAAAAACTAATGAGGGATGCCAAAATCTATCAGATTTATGAAG
GTACTTCACAAATTCAAAGACTTATTGTAGCCCGTGAACACATTGACAAGTACAAAAATTAAAAAAATTA
CTGTAGAAATATTGAATAACTAGAACACAAGCCACTGTTTCAGCTCCAGAAAAAAGAAAGGGCTTTAACG
TTTTTTCCAGTGAAAACAAATCCTCTTATATTAAATCTAAGCAACTGCTTATTATAGTAGTTTATACTTT
TGCTTAACTCTGTTATGTCTCTTAAGCAGGTTTGGTTTTTATTAAAATGATGTGTTTTCTTTAGTACCAC
TTTACTTGAATTACATTAACCTAGAAAACTACATAGGTTATTTTGATCTCTTAAGATTAATGTAGCAGAA
ATTTCTTGGAATTTTATTTTTGTAATGACAGAAAAGTGGGCTTAGAAAGTATTCAAGATGTTACAAAATT
TACATTTAGAAAATATTGTAGTATTTGAATACTGTCAACTTGACAGTAACTTTGTAGACTTAATGGTATT
ATTAAAGTTCTTTTTATTGCAGTTTGGAAAGCATTTGTGAAACTTTCTGTTTGGCACAGAAACAGTCAAA
ATTTTGACATTCATATTCTCCTATTTTACAGCTACAAGAACTTTCTTGAAAATCTTATTTAATTCTGAGC
CCATATTTCACTTACCTTATTTAAAATAAATCAATAAAGCTTGCCTTAAATTATTTTTATATGACTGTTG
GTCTCTAGGTAGCCTTTGGTCTATTGTACACAATCTCATTTCATATGTTTGCATTTTGGCAAAGAACTTA
ATAAAATTGTTCAGTGCTTATTATCAT

| | | | |
|---|---|---|---|
| NOV20l, 12252113 SNP for | | | |
| CG96778-01 | SEQ ID NO: | | SNP is in the non coding |
| Protein Sequence | 348 | 421 aa | region |

MAAGFGRCCRVLRSISRFHWRSQHTKANRQREPGLGFSFEFTEQQKEFQATARKFAREEIIPVAAEYDKT
GEYPVPLIRRAWELGLMNTHIPENCGGLGLGTFDACLISEELAYGCTGVQTAIEGNSLGQMPIIIAGNDQ
QKKKYLGRMTEEPLMCAYCVTEPGAGSDVAGIKTKAEKKGDEYIINGQKMWITNGGKANWYFLLARSDPD
PKAPANKAFTGFIVEADTPGIQIGRKELNMGQRCSDTRGIVFEDVKVPKENVLIGDGAGFKVAMGAFDKT
RPVVAAGAVGLAQRALDEATKYALERKTFGKLLVEHQAISFMLAEMAMKVELARMSYQRAAWEVDSGRRN
TYYASIAKAFAGDIANQLATDAVQILGGNGFNTEYPVEKLMRDAKIYQIYEGTSQIQRLIVAREHIDKYK
N

A ClustalW comparison of the above protein sequences yields the following sequence alignment shown in Table 20B.

TABLE 20B
Comparison of the NOV20 protein sequences.

| | |
|---|---|
| NOV20a | -----MAAGFGRCCR----VLRSISRFHWRSQHTKANRQREPGLGFSFEFTEQQKEFQAT |
| NOV20b | -----MAAGFGRCCR----VLRSISRFHWRSQHTKANRQREPGLGFSFEFTEQQKEFQAT |
| NOV20c | TRSPTMAAGFGRCCR----VLRSISRFHWRSQHTKANRQREPGLGFSFEFTEQQKEFQAT |
| NOV20d | TRSPTMAAGFGRCCR----VLRSISRFHWRSQHTKANRQREPGLGFSFEFTEQQKEFQAT |
| NOV20e | TRSPTMAAGFGRCCRCSLQVLRSISRFHWRSQHTKANRQREPGLGFSFEFTEQQKEFQAT |
| NOV20f | ----------------------------------------------TRSFTEQQKEFQAT |
| NOV20g | -----MAAGFGRCCRCSLQVLRSISRFHWRSQHTKANRQREPGLGFSFEFTEQQKEFQAT |
| NOV20a | ARKFAREEIIPVAAEYDKTGEYPVPLIRRAWELGLMNTHIPENCDYSVCPLLEACTLYLD |
| NOV20b | ARKFAREEIIPVAAEYDKTGEYPVPLIRRAWELGLMNTHIPEN-----C |
| NOV20c | ARKFAREEIIPVAAEYDKTGEYPVPLIRRAWELGLMNTHIPENCG |
| NOV20d | ARKFAREEIIPVAAEYDKTGEYPVPLIRRAWELGLMNTHIPENCDYSVCPLLEACTLYLD |
| NOV20e | ARKFAREEIIPVAAEYDKTGEYPVPLIRRAWELGLMNTHIPENCG |
| NOV20f | ARKFAREEIIPVAAEYDKTGEYPVPLIRRAWELCLMNTHIPENCG |
| NOV20g | ARKFAREEIIPVAAEYDKTGEYPVPLIRRAWELGLMNTHIPENCG |
| NOV20a | AFFLLLTGSNLNLHLNLGGLGLGTFDACLISEELAYGCTGVQTAIEGNSLGQMPIIIAGN |
| NOV20b | G-----------------GLGLGTFDACLISEELAYGCTGVQTAIEGNSLGQMPIIIAGN |
| NOV20c | G------------------LCLCTFDACLISEELAYGCTGVQTAIEGNSLGQMPIIIAGN |
| NOV20d | AFFLLLTGSNLNLHLNLGGLCLGTFDACLISEELAYGCTGVQTAIEGNSLGQMPIIIAGN |
| NCV20e | G------------------LGLGTFDACLISEELAYGCTGVQTAIEGNSLGQMPIIIAGN |
| NOV20f | ------------------GLGLGTFDACLISEELAYGCTGVQTAIECNSLGQMPIIIACN |
| NOV20g | C------------------LGLGTFDACLISEELAYGCTGVQTAIEGNSLGQMPIIIAGN |
| NOV20a | DQQKKKYLGRMTEEPLMCAYCVTEPGAGSDVAGIKTKAEKKGDEYIINGQKMWITNGGKA |
| NOV20b | DQQKKKYLGRMTEEPLMCAYCVTEPGAGSDVAGIKTKAEKKGDEYIINGQKMWITNGGKA |
| NOV20c | DQQKKKYLGRMTEEPLMCAYCVTEPGAGSDVAGIKTKAEKKGDEYIINGQKMWITNGGKA |
| NOV20d | DQQKKKYLGRMTEEPLMCAYCVTEPGAGSDVAGIKTKAEKKGDEYIINGQKMWITNGGKA |
| NOV20e | DQQKKKYLGRMTEEPLMCAYCVTEPGAGSDVAGIKTKAEKKGDEYIINGQKMWITNGGKA |
| NOV20F | DQQKKKYLGRMTEEPLMCAYCVTEPGAGSDVAGIKTKAEKKGDEYIINGQKMWITNGGKA |
| NOV20g | DQQKKKYLGRMTEEPLMCAYCVTEPGAGSDVAGIKTKAEKKGDEYIINGQKMWITNGGKA |
| NOV20a | NWYFLLARSDPDPKAPANKAFTGFIVEADTPGIQIGRKELNMGQRCSDTRGIVFEDVKVP |
| NOV20b | NWYFLLARSDPDPKAPANKAFTGFIVEADTPGIQIGRKELNMGQRCSDTRGIVFEDVKVP |
| NOV20c | NWYFLLARSDPDPKAPANKAFTGFIVEADTPGIQIGRKELNMGQRCSDTRGIVFEDVKVP |
| NOV20d | NWYFLLARSDPDPKAPANKAFTGFIVEADTPGIQIGRKELNMGQRCSDTRGIVFEDVKVP |
| NOV20e | NWYFLLARSDPDPKAPANKAFTGFIVEADTPGIQIGRKELNMGQRCSDTRGIVFEDVKVP |
| NOV20f | NWYFLLARSDPDPKAPANKAFTGFIVEADTPGIQIGRKELNMGQRCSDTRGIVFEDVKVP |
| NOV20g | NWYFLLARSDPDPKAPANKAFTGFIVEADTPGIQIGRKELNMGQRCSDTRGIVFEDVKVP |
| NOV20a | KENVLIGDGAGFKVAMGAFDKTRPVVAAGAVGLAQRALDEATKYALERKTFGKLLVEHQA |
| NOV20b | KENVLIGDGAGFKVAMGAFDKTRPVVAAGAVGLAQRALDEATKYALERKTFGKLLVEHQA |
| NOV20c | KENVLIGDGAGFKVAMGAFDKTRPVVAAGAVGLAQRALDEATKYALERKTFGKLLVEHQA |
| NOV20d | KENVLIGDGAGFKVAMGAFDKTRPVVAAGAVGLAQRALDEATKYALERKTFGKLLVEHQA |
| NOV20e | KENVLIGDGAGFKVAMGAFDKTRPVVAAGAVGLAQRALDEATKYALERKTFGKLLVEHQA |
| NOV20f | KENVLIGDGAGFKVAMGAFDKTRPVVAAGAVGLAQRALDEATKYALERKTFGKLLVEHQA |
| NOV20g | KENVLIGDGAGFKVAMGAFDKTRPVVAAGAVGLAQRALDEATKYALERKTFGKLLVEHQA |
| NOV20a | ISFMLAEMAMKVELARMSYQRAAWEVDSGRRNTYYASIAKAFAGDIANQLATDAVQILGG |
| NOV20b | ISFMLAEMAMKVELARMSYQRAAWEVDSGRRNTYYASIAKAFAGDIANQLATDAVQILGG |
| NOV20c | ISFMLAEMAMKVELARMSYQRAAWEVDSGRRNTYYASIAKAFAGDIANQLATDAVQILGG |
| NOV20d | ISFMLAEMAMKVELARMSYQRAAWEVDSGRRNTYYASIAKAFAGDIANQLATDAVQILGG |
| NOV20e | ISFMLAEMAMKVELARMSYQRAAWEVDSGRRNTYYASIAKAFAGDIANQLATDAVQILGG |
| NOV20f | ISFMLAEMAMKVELARMSYQRAAWEVDSGRRNTYYASIAKAFAGDIANQLATDAVQILGG |
| NOV20g | ISFMLAEMAMKVELARMSYQRAAWEVDSGRRNTYYASIAKAFAGDIANQLATDAVQILGG |
| NOV20a | NGFNTEYPVEKLMRDAKIYQIYEGTSQIQRLIVAREHIDKYKN |
| NOV20b | NGFNTEYPVEKLMRDAKIYQIYEGTSQIQRLIVAREHIDKYKN |
| NOV20c | NGFNTEYPVEKLMRDAKIYQIYEGTSQIQRLIVAREHIDKYKNVDG |
| NOV20d | NGFNTEYPVEKLMRDAKIYQIYEGTSQIQRLIVAREHIDKYKNVDG |
| NOV20e | NGFNTEYPVEKLMRDAKIYQIYEGTSQIQRLIVAREHIDKYKNVDG |
| NOV20f | NGFNTEYPVEKLMRDAKIYQIYEGTSQIQRLIVAREHIVDG |
| NOV20g | NGFNTEYPVEKLMRDAKIYQIYEGTSQIQRLIVAREHIDKYKN |
| NOV20a | (SEQ ID NO: 326) |
| NOV20b | (SEQ ID NO: 328) |
| NOV20c | (SEQ ID NO: 330) |
| NOV20d | (SEQ ID NO: 332) |
| NOV20e | (SEQ ID NO: 334) |
| NOV20f | (SEQ ID NO: 336) |
| NOV20g | (SEQ ID NO: 338) |

Further analysis of the NOV20a protein yielded the following properties shown in Table 20C.

TABLE 20C
Protein Sequence Properties NOV20a

| SignalP | No Known Signal Sequence Predicted | | | |
|---|---|---|---|---|
| analysis: | | | | |
| PSORT II | PSG: a new signal peptide prediction method | | | |
| analysis: | N-region: length 10; pos. chg 2; neg. chg 0 | | | |
| | H-region: length 2; peak value −5.86 | | | |
| | PSG score: −10.26 | | | |
| | GvH: von Heijne's method for signal seq. recognition | | | |
| | GvH score (threshold: −2.1): −9.99 | | | |
| | possible cleavage site: between 14 and 15 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 1 | | | |
| | Number of TMS(s) for threshold 0.5: 0 | | | |
| | PERIPHERAL Likelihood = 3.39 (at 126) | | | |
| | ALOM score: −0.90 (number of TMSs: 0) | | | |
| | MITDISC: discrimination of mitochondrial targeting seq | | | |
| | R content: | 7 | Hyd Moment(75): | 6.98 |
| | Hyd Moment(95): | 9.53 | G content: | 2 |
| | D/E content: | 1 | S/T content: | 4 |

```
Score: 2.17
Gavel: prediction of cleavage sites for mitochondrial preseq
R-2 motif at 39 NRQ|RE
NUCDISC: discrimination of nuclear localization signals
pat4: none
pat7: none
bipartite: none
content of basic residues: 12.1%
NLS Score: −0.47
KDEL: ER retention motif in the C-terminus: none
ER Membrane Retention Signals:
KKXX-like motif in the C-terminus: DKYK
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs:
Leucine zipper pattern (PS00029): *** found ***
LEACTLYLDAFFLLLTGSNLNL at 103
none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 94.1
COIL: Lupas's algorithm to detect coiled-coil regions
total: 0 residues
Final Results (k = 9/23):
91.3%: mitochondrial
  4.3%: nuclear
  4.3%: peroxisomal
>> prediction for CG96778-02 is mit (k = 23)
```

A search of the NOV20a protein against the Geneseq database, a proprietary database that contains sequences published in patents and patent publication, yielded several homologous proteins shown in Table 20D.

TABLE 20D
Geneseq Results for NOV20a

| | | NOV20a | Identities/ | |
|---|---|---|---|---|
| | | Residues/ | Similarities for | |
| Geneseq | Protein/Organism/Length [Patent | Match | the Matched | Expect |
| Identifier | #, Date] | Residues | Region | Value |
| AAG66900 | Human medium chain acyl-CoA | 1 . . . 454 | 414/454 (91%) | 0.0 |
| | dehydrogenase (ACADM) - Homo | 1 . . . 421 | 417/454 (91%) | |
| | sapiens, 421 aa. [WO200177336-A2, | | | |
| | 18 OCT. 2001] | | | |
| ABB61315 | Drosophila melanogaster polypeptide | 10 . . . 453 | 286/444 (64%) | e−164 |
| | SEQ ID NO 10737 - Drosophila | 13 . . . 416 | 338/444 (75%) | |
| | melanogaster, 419 aa. | | | |
| | [WO200171042-A2, 27 SEP. 2001] | | | |
| AAU44325 | Propionibacterium acnes | 34 . . . 447 | 153/417 (36%) | 1e−69 |
| | immunogenic protein #5221 - | 2 . . . 380 | 227/417 (53%) | |
| | Propionibacterium acnes, 386 aa. | | | |
| | [WO200181581-A2, 01 NOV. 2001] | | | |
| ABB60897 | Drosophila melanogaster polypeptide | 42 . . . 452 | 147/412 (35%) | 8e−62 |
| | SEQ ID NO 9483 - Drosophila | 28 . . . 403 | 214/412 (51%) | |
| | melanogaster, 405 aa. | | | |
| | [WO200171042-A2, 27 SEP. 2001] | | | |
| ABP10124 | Human ORFX protein sequence SEQ | 324 . . . 443 | 109/120 (90%) | 3e−56 |
| | ID NO: 20230 - Homo sapiens, 120 | 1 . . . 120 | 115/120 (95%) | |
| | aa. [WO200192523-A2, 06 DEC. | | | |
| | 2001] | | | |

In a BLAST search of public sequence databases, the NOV20a protein was found to have homology to the proteins shown in the BLASTP data in Table 20E.

TABLE 20E
Public BLASTP Results for NOV20a

| | | NOV20a | Identities/ | |
|---|---|---|---|---|
| Protein | | Residues/ | Similarities for | |
| Accession | | Match | the Matched | Expect |
| Number | Protein/Organism/Length | Residues | Portion | Value |
| P11310 | Acyl-CoA dehydrogenase, medium- | 1 . . . 454 | 421/454 (92%) | 0.0 |
| | chain specific, mitochondrial | 1 . . . 421 | 421/454 (92%) | |
| | precursor (EC 1.3.99.3) (MCAD) - | | | |
| | Homo sapiens (Human), 421 aa. | | | |
| Q8HXY8 | Medium-chain acyl-CoA | 1 . . . 454 | 405/454 (89%) | 0.0 |
| | dehydrogenase - Macaca fascicularis | 1 . . . 421 | 415/454 (91%) | |
| | (Crab eating macaque) (Cynomolgus | | | |
| | monkey), 421 aa. | | | |
| P45952 | Acyl-CoA dehydrogenase, medium- | 1 . . . 454 | 370/454 (81%) | 0.0 |
| | chain specific, mitochondrial | 1 . . . 421 | 399/454 (87%) | |
| | precursor (EC 1.3.99.3) (MCAD) - | | | |
| | Mus musculus (Mouse), 421 aa. | | | |
| Q91WS8 | Acetyl-coenzyme A dehydrogenase, | 1 . . . 454 | 369/454 (81%) | 0.0 |
| | medium chain - Mus musculus | 1 . . . 421 | 398/454 (87%) | |
| | (Mouse), 421 aa. | | | |
| P08503 | Acyl-CoA dehydrogenase, medium- | 1 . . . 454 | 364/454 (80%) | 0.0 |
| | chain specific, mitochondrial | 1 . . . 421 | 396/454 (87%) | |
| | precursor (EC 1.3.99.3) (MCAD) - | | | |
| | Rattus norvegicus (Rat), 421 aa. | | | |

PFam analysis predicts that the NOV20a protein contains the domains shown in the Table 20F.

TABLE 20F
Domain Analysis of NOV20a

| | | Identities/ | |
|---|---|---|---|
| | NOV20a | Similarities | |
| Pfam | Match | for the | Expect |
| Domain | Region | Matched Region | Value |
| Acyl-CoA_dh_N | 41 . . . 190 | 51/165 (31%) | 1.8e−45 |
| | | 130/165 (79%) | |
| Acyl-CoA_dh_M | 192 . . . 296 | 64/106 (60%) | 8e−69 |
| | | 104/106 (98%) | |
| Acyl-CoA_dh | 300 . . . 449 | 78/162 (48%) | 7.3e−68 |
| | | 126/162 (78%) | |
| HpaB | 148 . . . 454 | 63/399 (16%) | 0.023 |
| | | 203/399 (51%) | |

### Example B

### Sequencing Methodology and Identification of NOVX Clones

1. GeneCalling™ Technology: This is a proprietary method of performing differential gene expression profiling between two or more samples developed at CuraGen and described by Shimkets, et al., “Gene expression analysis by transcript profiling coupled to a gene database query” Nature Biotechnology 17:198-803 (1999). cDNA was derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then digested with up to as many as 120 pairs of restriction enzymes and pairs of linker-adaptors specific for each pair of restriction enzymes were ligated to the appropriate end. The restriction digestion generates a mixture of unique cDNA gene fragments. Limited PCR amplification is performed with primers homologous to the linker adapter sequence where one primer is biotinylated and the other is fluorescently labeled. The doubly labeled material is isolated and the fluorescently labeled single strand is resolved by capillary gel electrophoresis. A computer algorithm compares the electropherograms from an experimental and control group for each of the restriction digestions. This and additional sequence-derived information is used to predict the identity of each differentially expressed gene fragment using a variety of genetic databases. The identity of the gene fragment is confirmed by additional, gene-specific competitive PCR or by isolation and sequencing of the gene fragment.

2. SeqCalling™ Technology: cDNA was derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then sequenced using CuraGen's proprietary SeqCalling technology. Sequence traces were evaluated manually and edited for corrections if appropriate. cDNA sequences from all samples were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

3. PathCalling™ Technology: The NOVX nucleic acid sequences are derived by laboratory screening of cDNA library by the two-hybrid approach. cDNA fragments covering either the full length of the DNA sequence, or part of the sequence, or both, are sequenced. In silico prediction was based on sequences available in CuraGen Corporation's proprietary sequence databases or in the public human sequence databases, and provided either the full length DNA sequence, or some portion thereof.

The laboratory screening was performed using the methods summarized below:

cDNA libraries were derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then directionally cloned into the appropriate two-hybrid vector (Gal4-activation domain (Gal4-AD) fusion). Such cDNA libraries as well as commercially available cDNA libraries from Clontech (Palo Alto, Calif.) were then transferred from E. coli into a CuraGen Corporation proprietary yeast strain (disclosed in U.S. Pat. Nos. 6,057,101 and 6,083,693, incorporated herein by reference in their entireties).

Gal4-binding domain (Gal4-BD) fusions of a CuraGen Corportion proprietary library of human sequences was used to screen multiple Gal4-AD fusion cDNA libraries resulting in the selection of yeast hybrid diploids in each of which the Gal4-AD fusion contains an individual cDNA. Each sample was amplified using the polymerase chain reaction (PCR) using non-specific primers at the cDNA insert boundaries. Such PCR product was sequenced; sequence traces were evaluated manually and edited for corrections if appropriate. cDNA sequences from all samples were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

Physical clone: the cDNA fragment derived by the screening procedure, covering the entire open reading frame is, as a recombinant DNA, cloned into pACT2 plasmid (Clontech) used to make the cDNA library. The recombinant plasmid is inserted into the host and selected by the yeast hybrid diploid generated during the screening procedure by the mating of both CuraGen Corporation proprietary yeast strains N106′ and YULH (U.S. Pat. Nos. 6,057,101 and 6,083,693).

4. RACE: Techniques based on the polymerase chain reaction such as rapid amplification of cDNA ends (RACE), were used to isolate or complete the predicted sequence of the cDNA of the invention. Usually multiple clones were sequenced from one or more human samples to derive the sequences for fragments. Various human tissue samples from different donors were used for the RACE reaction. The sequences derived from these procedures were included in the SeqCalling Assembly process described in preceding paragraphs.

5. Exon Linking: The NOVX target sequences identified in the present invention were subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The PCR product derived from exon linking was cloned into the pCR2.1 vector from Invitrogen. The resulting bacterial clone has an insert covering the entire open reading frame cloned into the pCR2.1 vector. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported herein.

6. Physical Clone: Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

The PCR product derived by exon linking, covering the entire open reading frame, was cloned into the pCR2.1 vector from Invitrogen to provide clones used for expression and screening purposes.

### Example C

### Quantitative Expression Analysis of Clones in Various Cells and Tissues

The quantitative expression of various NOV genes was assessed using microtiter plates containing RNA samples from a variety of normal and pathology-derived cells, cell lines and tissues using real time quantitative PCR (RTQ-PCR) performed on an Applied Biosystems (Foster City, Calif.) ABI PRISM® 7700 or an ABI PRISM® 7900 HT Sequence Detection System.

RNA integrity of all samples was determined by visual assessment of agarose gel electropherograms using 28S and 18S ribosomal RNA staining intensity ratio as a guide (2:1 to 2.5:1 28s:18s) and the absence of low molecular weight RNAs (degradation products). Control samples to detect genomic DNA contamination included RTQ-PCR reactions run in the absence of reverse transcriptase using probe and primer sets designed to amplify across the span of a single exon.

RNA samples were normalized in reference to nucleic acids encoding constitutively expressed genes (i.e., β-actin and GAPDH). Alternatively, non-normalized RNA samples were converted to single strand cDNA (sscDNA) using Superscript II (Invitrogen Corporation, Carlsbad, Calif., Catalog No. 18064-147) and random hexamers according to the manufacturer's instructions. Reactions containing up to 10 μg of total RNA in a volume of 20 μl or were scaled up to contain 50 μg of total RNA in a volume of 100 l and were incubated for 60 minutes at 42° C. sscDNA samples were then normalized in reference to nucleic acids as described above.

Probes and primers were designed according to Applied Biosystems Primer Express Software package (version I for Apple Computer's Macintosh Power PC) or a similar algorithm using the target sequence as input. Default reaction condition settings and the following parameters were set before selecting primers: 250 nM primer concentration; 58°-60° C. primer melting temperature (Tm) range; 59° C. primer optimal Tm; 2° C. maximum primer difference (if probe does not have 5′ G, probe Tₘ must be 10° C. greater than primer Tₘ; and 75 bp to 100 bp amplicon size. The selected probes and primers were synthesized by Synthegen (Houston, Tex.). Probes were double purified by HPLC to remove uncoupled dye and evaluated by mass spectroscopy to verify coupling of reporter and quencher dyes to the 5′ and 3′ ends of the probe, respectively. Their final concentrations were: 900 nM forward and reverse primers, and 200 nM probe.

Normalized RNA was spotted in individual wells of a 96 or 384-well PCR plate (Applied Biosystems, Foster City, Calif.). PCR cocktails included a single gene-specific probe and primers set or two multiplexed probe and primers sets. PCR reactions were done using TaqMan® One-Step RT-PCR Master Mix (Applied Biosystems, Catalog No. 4313803) following manufacturer's instructions. Reverse transcription was performed at 48° C. for 30 minutes followed by amplification/PCR cycles: 95° C. 10 min, then 40 cycles at 95° C. for 15 seconds, followed by 60° C. for 1 minute. Results were recorded as CT values (cycle at which a given sample crosses a threshold level of fluorescence) and plotted using a log scale, with the difference in RNA concentration between a given sample and the sample with the lowest CT value being represented as 2 to the power of delta CT. The percent relative expression was the reciprocal of the RNA difference multiplied by 100. CT values below 28 indicate high expression, between 28 and 32 indicate moderate expression, between 32 and 35 indicate low expression and above 35 reflect levels of expression that were too low to be measured reliably.

Normalized sscDNA was analyzed by RTQ-PCR using 1× TaqMan® Universal Master mix (Applied Biosystems; catalog No.4324020), following the manufacturer's instructions. PCR amplification and analysis were done as described above.

Panels 1, 1.1, 1.2, and 1.3D

Panels 1, 1.1, 1.2 and 1.3D included 2 control wells (genomic DNA control and chemistry control) and 94 wells of cDNA samples from cultured cell lines and primary normal tissues. Cell lines were derived from carcinomas (ca) including: lung, small cell (s cell var), non small cell (non-s or non-sm); breast; melanoma; colon; prostate; glioma (glio), astrocytoma (astro) and neuroblastoma (neuro); squamous cell (squam); ovarian; liver; renal; gastric and pancreatic from the American Type Culture Collection (ATCC, Bethesda, Md.). Normal tissues were obtained from individual adults or fetuses and included: adult and fetal skeletal muscle, adult and fetal heart, adult and fetal kidney, adult and fetal liver, adult and fetal lung, brain, spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose. The following abbreviations are used in reporting the results: metastasis (met); pleural effusion (pl. eff or pl effusion) and * indicates established from metastasis.

GENERAL_SCREENING_PANEL_V1.4, V1.5, V1.6 AND 1.7

Panels 1.4, 1.5, 1.6 and 1.7 were as described for Panels 1, 1.1, 1.2 and 1.3D, above except that normal tissue samples were pooled from 2 to 5 different adults or fetuses.

Panels 2D, 2.2, 2.3, and 2.4

Panels 2D, 2.2, 2.3 and 2.4 included 2 control wells and 94 wells containing RNA or cDNA from human surgical specimens procured through the National Cancer Institute's Cooperative Human Tissue Network (CHTN) or the National Disease Research Initiative (NDRI), Ardais (Lexington, Mass.) or Clinomics BioSciences (Frederick, Md.). Tissues included human malignancies and in some cases matched adjacent normal tissue (NAT). Information regarding histopathological assessment of tumor differentiation grade as well as the clinical stage of the patient from which samples were obtained was generally available. Normal tissue RNA and cDNA samples were purchased from various commercial sources such as Clontech (Palo Alto, Calif.), Research Genetics and Invitrogen (Carlsbad, Calif.).

HASS Panel V1.0

The HASS Panel v1.0 included 93 cDNA samples and two controls including: 81 samples of cultured human cancer cell lines subjected to serum starvation, acidosis and anoxia according to established procedures for various lengths of time; 3 human primary cells; 9 malignant brain cancers (4 medulloblastomas and 5 glioblastomas); and 2 controls. Cancer cell lines (ATCC) were cultured using recommended conditions and included: breast, prostate, bladder, pancreatic and CNS. Primary human cells were obtained from Clonetics (Walkersville, Md.). Malignant brain samples were gifts from the Henry Ford Cancer Center.

Ardais Panel V1.0

The ARDAIS Panel v1.0 included 2 controls and 22 test samples including: human lung adenocarcinomas, lung squamous cell carcinomas, and in some cases matched adjacent normal tissues (NAT) obtained from Ardais. Unmatched malignant and non-malignant RNA samples from lungs with gross histopathological assessment of tumor differentiation grade and stage and clinical state of the patient were obtained from Ardais.

Ardais Prostate V1.0

ARDAIS Prostate v10.0 panel included 2 controls and 68 test samples of human prostate malignancies and in some cases matched adjacent normal tissues (NAT) obtained from Ardais. RNA from unmatched malignant and non-malignant prostate samples with gross histopathological assessment of tumor differentiation grade and stage and clinical state of the patient were also obtained from Ardais.

Ardais Kidney V1.0

ARDAIS Kidney v1.0 panel included 2 control wells and 44 test samples of human renal cell carcinoma and in some cases matched adjacent normal tissue (NAT) obtained from Ardais. RNA from unmatched renal cell carcinoma and normal tissue with gross histopathological assessment of tumor differentiation grade and stage and clinical state of the patient were also obtained from Ardais.

Panels 3D, 3.1 and 3.2

Panels 3D, 3.1, and 3.2 included two controls, 92 cDNA samples of cultured human cancer cell lines and 2 samples of human primary cerebellum. Cell lines (ATCC, National Cancer Institute (NCI), German tumor cell bank) were cultured as recommended and were derived from: squamous cell carcinoma of the tongue, melanoma, sarcoma, leukemia, lymphoma, and epidermoid, bladder, pancreas, kidney, breast, prostate, ovary, uterus, cervix, stomach, colon, lung and CNS carcinomas.

Panels 4D, 4R, and 4.1D

Panels 4D, 4R, and 4.1 D included 2 control wells and 94 test samples of RNA (Panel 4R) or cDNA (Panels 4D and 4.1 D) from human cell lines or tissues related to inflammatory conditions. Controls included total RNA from normal tissues such as colon, lung (Stratagene, La Jolla, Calif.), thymus and kidney (Clontech, Palo Alto, Calif.). Total RNA from cirrhotic and lupus kidney was obtained from BioChain Institute, Inc., (Hayward, Calif.). Crohn's intestinal and ulcerative colitis samples were obtained from the National Disease Research Interchange (NDRI, Philadelphia, Pa.). Cells purchased from Clonetics (Walkersville, Md.) included: astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, and human umbilical vein endothelial. These primary cell types were activated by incubating with various cytokines (IL-1 beta ˜1-5 ng/ml, TNF alpha ˜5-10 ng/ml, IFN gamma-20-50 ng/ml, IL-4 ˜5-10 ng/ml, IL-9 ˜5-10 ng/ml, IL-13 5-10 ng/ml) or combinations of cytokines as indicated. Starved endothelial cells were cultured in the basal media (Clonetics, Walkersville, Md.) with 0.1% serum.

Mononuclear cells were prepared from blood donations using Ficoll. LAK cells were cultured in culture media [DMEM, 5% FCS (Hyclone, Logan, Utah), 100 RM non essential amino acids (Gibco/Life Technologies, Rockville, Md.), 1 mM sodium pyruvate (Gibco), mercaptoethanol 5.5×10⁻⁵ M (Gibco), and 10 mM Hepes (Gibco)] and interleukin 2 for 4-6 days. Cells were activated with 10-20 ng/ml PMA and 1-2 μg/ml ionomycin, 5-10 ng/ml IL-12, 20-50 ng/ml IFN gamma or 5-10 ng/ml IL-18 for 6 hours. In some cases, mononuclear cells were cultured for 4-5 days in culture media with ˜5 μg/ml PHA (phytohemagglutinin) or PWM (pokeweed mitogen; Sigma-Aldrich Corp., St. Louis, Mo.). Samples were taken at 24, 48 and 72 hours for RNA preparation. MLR (mixed lymphocyte reaction) samples were obtained by taking blood from two donors, isolating the mononuclear cells using Ficoll and mixing them 1:1 at a final concentration of ˜2×10⁶ cells/ml in culture media. The MLR samples were taken at various time points from 1-7 days for RNA preparation.

Monocytes were isolated from mononuclear cells using CD14 Miltenyi Beads, +ve VS selection columns and a Vario Magnet (Miltenyi Biotec, Auburn, Calif.) according to the manufacturer's instructions. Monocytes were differentiated into dendritic cells by culturing in culture media with 50 ng/ml GMCSF and 5 ng/ml IL-4 for 5-7 days. Macrophages were prepared by culturing monocytes for 5-7 days in culture media with ˜50 ng/ml 10% type AB Human Serum (Life technologies, Rockville, Md.) or MCSF (Macrophage colony stimulating factor; R&D, Minneapolis, Minn.). Monocytes, macrophages and dendritic cells were stimulated for 6 or 12-14 hours with 100 ng/ml lipopolysaccharide (LPS). Dendritic cells were also stimulated with 10 μg/ml anti-CD40 monoclonal antibody (Pharmingen, San Diego, Calif.) for 6 or 12-14 hours.

CD4+ lymphocytes, CD8+ lymphocytes and NK cells were also isolated from mononuclear cells using CD4, CD8 and CD56 Miltenyi beads, positive VS selection columns and a Vario Magnet (Miltenyi Biotec, Auburn, Calif.) according to the manufacturer's instructions. CD45+RA and CD45+RO CD4+ lymphocytes were isolated by depleting mononuclear cells of CD8+, CD56+, CD14+and CD19+cells using CD8, CD56, CD14 and CD 19 Miltenyi beads and positive selection. CD45RO Miltenyi beads were then used to separate the CD45+RO CD4+ lymphocytes from CD45+RA CD4+ lymphocytes. CD45+RA CD4+, CD45+RO CD4+and CD8+ lymphocytes were cultured in culture media at 106 cells/ml in culture plates precoated overnight with 0.5 μg/ml anti-CD28 (Pharmingen, San Diego, Calif.) and 3 □g/ml anti-CD3 (OKT3, ATCC) in PBS. After 6 and 24 hours, the cells were harvested for RNA preparation. To prepare chronically activated CD8+ lymphocytes, isolated CD8+ lymphocytes were activated for 4 days on anti-CD28, anti-CD3 coated plates and then harvested and expanded in culture media with IL-2 (1 ng/ml). These CD8+cells were activated again with plate bound anti-CD3 and anti-CD28 for 4 days and expanded as described above. RNA was isolated 6 and 24 hours after the second activation and after 4 days of the second expansion culture. Isolated NK cells were cultured in culture media with 1 ng/ml IL-2 for 4-6 days before RNA was prepared.

B cells were prepared from minced and sieved tonsil tissue (NDRI). Tonsil cells were pelleted and resupended at 10⁶ cells/ml in culture media. Cells were activated using 5 μg/ml PWM (Sigma-Aldrich Corp., St. Louis, Mo.) or ˜10 μg/ml anti-CD40 (Pharmingen, San Diego, Calif.) and 5-10 ng/ml IL-4. Cells were harvested for RNA preparation after 24, 48 and 72 hours.

To prepare primary and secondary Th1/Th2 and Tr1 cells, umbilical cord blood CD4+ lymphocytes (Poietic Systems, German Town, Md.) were cultured at 10⁵-10⁶cells/ml in culture media with IL-2 (4 ng/ml) in 6-well Falcon plates (precoated overnight with 10 μg/ml anti-CD28 (Pharmingen) and 2 μg/ml anti-CD3 (OKT3; ATCC) then washed twice with PBS).

To stimulate Th1 phenotype differentiation, IL-12 (5 ng/ml) and anti-IL4 (1 μg/ml) were used; for Th2 phenotype differentiation, IL-4 (5 ng/ml) and anti-IFN gamma (1 μg/ml) were used; and for Tr1 phenotype differentiation, IL-10 (5 ng/ml) was used. After 4-5 days, the activated Th1, Th2 and Tr1 lymphocytes were washed once with DMEM and expanded for 4-7 days in culture media with IL-2 (1 ng/ml). Activated Th1, Th2 and Tr1 lymphocytes were re-stimulated for 5 days with anti-CD28/CD3 and cytokines as described above with the addition of anti-CD95L (1 μg/ml) to prevent apoptosis. After 4-5 days, the Th1, Th2 and Tr1 lymphocytes were washed and expanded in culture media with IL-2 for 4-7 days. Activated Th1 and Th2 lymphocytes were maintained for a maximum of three cycles. RNA was prepared from primary and secondary Th1, Th2 and Tr1 after 6 and 24 hours following the second and third activations with plate-bound anti-CD3 and anti-CD28 mAbs and 4 days into the second and third expansion cultures.

Leukocyte cells lines Ramos, EOL-1, KU-812 were obtained from the ATCC. EOL-1 cells were further differentiated by culturing in culture media at 5×10⁵ cells/ml with 0.1 mM dbcAMP for 8 days, changing the media every 3 days and adjusting the cell concentration to 5×10⁵ cells/ml. RNA was prepared from resting cells or cells activated with PMA (10 ng/ml) and ionomycin (1 μg/ml) for 6 and 14 hours. RNA was prepared from resting CCD 1106 keratinocyte cell line (ATCC) or from cells activated with ˜5 ng/ml TNF alpha and 1 ng/ml IL-1 beta. RNA was prepared from resting NCI-H292, airway epithelial tumor cell line (ATCC) or from cells activated for 6 and 14 hours in culture media with 5 ng/ml IL-4, 5 ng/ml IL-9, 5 ng/ml IL-13, and 25 ng/ml IFN gamma.

RNA was prepared by lysing approximately 10⁷ cells/ml using Trizol (Gibco BRL) then adding 1/10 volume of bromochloropropane (Molecular Research Corporation, Cincinnati, Ohio), vortexing, incubating for 10 minutes at room temperature and then spinning at 14,000 rpm in a Sorvall SS34 rotor. The aqueous phase was placed in a 15 ml Falcon Tube and an equal volume of isopropanol was added and left at −20° C. overnight. The precipitated RNA was spun down at 9,000 rpm for 15 min and washed in 70% ethanol. The pellet was redissolved in 300 μl of RNAse-free water with 35 μl buffer (Promega, Madison, Wis.) 5 μl DTT, 7 μl RNAsin and 8 μl DNAse and incubated at 37° C. for 30 minutes to remove contaminating genomic DNA, extracted once with phenol chloroform and re-precipitated with 1/10 volume of 3 M sodium acetate and 2 volumes of 100% ethanol. The RNA was spun down, placed in RNAse free water and stored at −80° C.

AI Comprehensive Panel v1.0

Autoimmunity (AI) comprehensive panel v1.0 included two controls and 89 cDNA test samples isolated from male (M) and female (F) surgical and postmortem human tissues that were obtained from the Backus Hospital and Clinomics (Frederick, Md.). Tissue samples included: normal, adjacent (Adj); matched normal adjacent (match control); joint tissues (synovial (Syn) fluid, synovium, bone and cartilage, osteoarthritis (OA), rheumatoid arthritis (RA)); psoriatic; ulcerative colitis colon; Crohns disease colon; and emphysmatic, asthmatic, allergic and chronic obstructive pulmonary disease (COPD) lung.

AI.05 Chondrosarcoma

AI.05 chondrosarcoma plates included SW1353 cells (ATCC) subjected to serum starvation and treated for 6 and 18 h with cytokines that are known to induce MMP (1, 3 and 13) synthesis (e.g. IL1beta). These treatments included: IL-1β (10 ng/ml), IL-1β+TNF-α (50 ng/ml), IL-1β+Oncostatin (50 ng/ml) and PMA (100 ng/ml). Supernatants were collected and analyzed for MMP 1, 3 and 13 production. RNA was prepared from these samples using standard procedures.

Panels 5D and 5I

Panel 5D and 5I included two controls and cDNAs isolated from human tissues, human pancreatic islets cells, cell lines, metabolic tissues obtained from patients enrolled in the Gestational Diabetes study (described below), and cells from different stages of adipocyte differentiation, including differentiated (AD), midway differentiated (AM), and undifferentiated (U; human mesenchymal stem cells).

Gestational Diabetes study subjects were young (18-40 years), otherwise healthy women with and without gestational diabetes undergoing routine (elective) Caesarean section. Uterine wall smooth muscle (UT), visceral (Vis) adipose, skeletal muscle (SK), placenta (PI) greater omentum adipose (GO Adipose) and subcutaneous (SubQ) adipose samples (<1 cc) were collected, rinsed in sterile saline, blotted and flash frozen in liquid nitrogen. Patients included: Patient 2, an overweight diabetic Hispanic not on insulin; Patient 7-9, obese non-diabetic Caucasians with body mass index (BMI) greater than 30; Patient 10, an overweight diabetic Hispanic, on insulin; Patient 11, an overweight nondiabetic African American; and Patient 12, a diabetic Hispanic on insulin.

Differentiated adipocytes were obtained from induced donor progenitor cells (Clonetics, Walkersville, Md.). Differentiated human mesenchymal stem cells (HuMSCs) were prepared as described in Mark F. Pittenger, et al., Multilineage Potential of Adult Human Mesenchymal Stem Cells Science Apr. 2, 1999: 143-147. mRNA was isolated and sscDNA was produced from Trizol lysates or frozen pellets. Human cell lines (ATCC, NCI or German tumor cell bank) included: kidney proximal convoluted tubule, uterine smooth muscle cells, small intestine, liver HepG2 cancer cells, heart primary stromal cells and adrenal cortical adenoma cells. Cells were cultured, RNA extracted and sscDNA was produced using standard procedures

Panel 5I also contains pancreatic islets (Diabetes Research Institute at the University of Miami School of Medicine).

Human Metabolic RTQ-PCR Panel

Human Metabolic RTQ-PCR Panel included two controls (genomic DNA control and chemistry control) and 211 cDNAs isolated from human tissues and cell lines relevant to metabolic diseases. This panel identifies genes that play a role in the etiology and pathogenesis of obesity and/or diabetes. Metabolic tissues including placenta (PI), uterine wall smooth muscle (Ut), visceral adipose, skeletal muscle (Sk) and subcutaneous (SubQ) adipose were obtained from the Gestational Diabetes study (described above). Included in the panel are: Patients 7 and 8, obese non-diabetic Caucasians; Patient 12 a diabetic Caucasian with unknown BMI, on insulin (treated); Patient 13, an overweight diabetic Caucasian, not on insulin (untreated); Patient 15, an obese, untreated, diabetic Caucasian; Patient 17 and 25, untreated diabetic Caucasians of normal weight; Patient 18, an obese, untreated, diabetic Hispanic; Patient 19, a non-diabetic Caucasian of normal weight; Patient 20, an overweight, treated diabetic Caucasian; Patient 21 and 23, overweight non-diabetic Caucasians; Patient 22, a teated diabetic Caucasian of normal weight; Patient 23, an overweight non-diabetic Caucasian; and Patients 26 and 27, obese, treated, diabetic Caucasians.

Total RNA was isolated from metabolic tissues including: hypothalamus, liver, pancreas, pancreatic islets, small intestine, psoas muscle, diaphragm muscle, visceral (Vis) adipose, subcutaneous (SubQ) adipose and greater omentum (Go) from 12 Type II diabetic (Diab) patients and 12 non diabetic (Norm) at autopsy. Control diabetic and non-diabetic subjects were matched where possible for: age; sex, male (M); female (F); ethnicity, Caucasian (CC); Hispanic (HI); African American (AA); Asian (AS); and BMI, 20-25 (Low BM), 26-30 (Med BM) or overweight (Overwt), BMI greater than 30 (Hi BMI) (obese).

RNA was extracted and ss cDNA was produced from cell lines (ATCC) by standard methods.

CNS Panels

CNS Panels CNSD.01, CNS Neurodegeneration V1.0 and CNS Neurodegeneration V2.0 included two controls and 46 to 94 test cDNA samples isolated from postmortem human brain tissue obtained from the Harvard Brain Tissue Resource Center (McLean Hospital). Brains were removed from calvaria of donors between 4 and 24 hours after death, and frozen at −80° C. in liquid nitrogen vapor.

Panel CNSD.01

Panel CNSD.01 included two specimens each from: Alzheimer's disease, Parkinson's disease, Huntington's disease, Progressive Supernuclear Palsy (PSP), Depression, and normal controls. Collected tissues included: cingulate gyrus (Cing Gyr), temporal pole (Temp Pole), globus palladus (Glob palladus), substantia nigra (Sub Nigra), primary motor strip (Brodman Area 4), parietal cortex (Brodman Area 7), prefrontal cortex (Brodman Area 9), and occipital cortex (Brodman area 17). Not all brain regions are represented in all cases.

Panel CNS Neurodegeneration V1.0

The CNS Neurodegeneration V1.0 panel included: six Alzheimer's disease (AD) brains and eight normals which included no dementia and no Alzheimer's like pathology (control) or no dementia but evidence of severe Alzheimer's like pathology (Control Path), specifically senile plaque load rated as level 3 on a scale of 0-3; 0 no evidence of plaques, 3 severe AD senile plaque load. Tissues collected included: hippocampus, temporal cortex (Brodman Area 21), parietal cortex (Brodman area 7), occipital cortex (Brodman area 17) superior temporal cortex (Sup Temporal Ctx) and inferior temporal cortex (Inf Temproal Ctx).

Gene expression was analyzed after normalization using a scaling factor calculated by subtracting the Well mean (CT average for the specific tissue) from the Grand mean (average CT value for all wells across all runs). The scaled CT value is the result of the raw CT value plus the scaling factor.

Panel CNS Neurodegeneration V2.0

The CNS Neurodegeneration V2.0 panel included sixteen cases of Alzheimer's disease (AD) and twenty-nine normal controls (no evidence of dementia prior to death) including fourteen controls (Control) with no dementia and no Alzheimer's like pathology and fifteen controls with no dementia but evidence of severe Alzheimer's like pathology (AH3), specifically senile plaque load rated as level 3 on a scale of 0-3; 0 no evidence of plaques, 3 severe AD senile plaque load. Tissues from the temporal cortex (Brodman Area 21) included the inferior and superior temporal cortex that was pooled from a given individual (Inf & Sup Temp Ctx Pool).

A. CG103910-02: BMP7.

Expression of gene CG103910-02 was assessed using the primer-probe set Ag7249, described in Table AA. Results of the RTQ-PCR runs are shown in Tables AB and AC.

TABLE AA

Probe Name Ag7249

| | | | | |
|---|---|---|---|---|
| | | | | SEQ ID |
| Primers | Sequences | Length | Start Position | No |

| | | | | |
|---|---|---|---|---|
| Forward | 5′-aacgtggcaggtccactt-3′ | 18 | 949 | 349 |
| Probe | TET-5′-atcaacccggaaacggtgcccaa-3′- | 23 | 968 | 350 |
| | TAMRA | | | |
| Reverse | 5′-catcgaagtagaggacggagat-3′ | 22 | 1022 | 351 |

TABLE AB
General_screening_panel_v1.7

| Tissue Name | A |
|---|---|
| Adipose | 0.2 |
| HUVEC | 0.0 |
| Melanoma* Hs688(A).T | 0.0 |
| Melanoma* Hs688(B).T | 10.7 |
| Melanoma (met) SK-MEL-5 | 1.5 |
| Testis | 0.7 |
| Prostate ca. (bone met) PC-3 | 0.0 |
| Prostate ca. DU145 | 0.0 |
| Prostate pool | 0.0 |
| Uterus pool | 0.0 |
| Ovarian ca. OVCAR-3 | 12.1 |
| Ovarian ca. (ascites) SK-OV-3 | 0.0 |
| Ovarian ca. OVCAR-4 | 62.0 |
| Ovarian ca. OVCAR-5 | 0.0 |
| Ovarian ca. IGROV-1 | 100.0 |
| Ovarian ca. OVCAR-8 | 0.0 |
| Ovary | 0.0 |
| Breast ca. MCF-7 | 32.8 |
| Breast ca. MDA-MB-231 | 0.0 |
| Breast ca. BT-549 | 0.0 |
| Breast ca. T47D | 0.0 |
| Breast pool | 0.0 |
| Trachea | 3.1 |
| Lung | 0.8 |
| Fetal Lung | 2.6 |
| Lung ca. NCI-N417 | 0.0 |
| Lung ca. LX-1 | 10.2 |
| Lung ca. NCI-H146 | 0.0 |
| Lung ca. SHP-77 | 0.0 |
| Lung ca. NCI-H23 | 15.5 |
| Lung ca. NCI-H460 | 0.3 |
| Lung ca. HOP-62 | 0.0 |
| Lung ca. NCI-H522 | 19.2 |
| Lung ca. DMS-114 | 3.0 |
| Liver | 0.0 |
| Fetal Liver | 0.0 |
| Kidney pool | 1.4 |
| Fetal Kidney | 4.7 |
| Renal ca. 786-0 | 0.0 |
| Renal ca. A498 | 0.0 |
| Renal ca. ACHN | 0.0 |
| Renal ca. UO-31 | 0.0 |
| Renal ca. TK-10 | 0.0 |
| Bladder | 1.2 |
| Gastric ca. (liver met.) NCI-N87 | 1.5 |
| Stomach | 0.0 |
| Colon ca. SW-948 | 6.2 |
| Colon ca. SW480 | 0.9 |
| Colon ca. (SW480 met) SW620 | 61.1 |
| Colon ca. HT29 | 0.0 |
| Colon ca. HCT-116 | 10.3 |
| Colon cancer tissue | 0.9 |
| Colon ca. SW1116 | 6.2 |
| Colon ca. Colo-205 | 19.6 |
| Colon ca. SW-48 | 1.2 |
| Colon | 0.0 |
| Small Intestine | 0.0 |
| Fetal Heart | 4.3 |
| Heart | 0.0 |
| Lymph Node pool 1 | 0.0 |
| Lymph Node pool 2 | 0.3 |
| Fetal Skeletal Muscle | 1.1 |
| Skeletal Muscle pool | 0.0 |
| Skeletal Muscle | 0.0 |
| Spleen | 0.3 |
| Thymus | 0.0 |
| CNS cancer (glio/astro) SF-268 | 0.0 |
| CNS cancer (glio/astro) T98G | 8.5 |
| CNS cancer (neuro; met) SK-N-AS | 0.0 |
| CNS cancer (astro) SF-539 | 0.2 |
| CNS cancer (astro) SNB-75 | 3.9 |
| CNS cancer (glio) SNB-19 | 0.2 |
| CNS cancer (glio) SF-295 | 8.4 |
| Brain (Amygdala) | 2.5 |
| Brain (Cerebellum) | 3.3 |
| Brain (Fetal) | 6.3 |
| Brain (Hippocampus) | 2.3 |
| Cerebral Cortex pool | 1.4 |
| Brain (Substantia nigra) | 0.0 |
| Brain (Thalamus) | 1.9 |
| Brain (Whole) | 7.0 |
| Spinal Cord | 1.4 |
| Adrenal Gland | 0.3 |
| Pituitary Gland | 0.0 |
| Salivary Gland | 0.3 |
| Thyroid | 6.9 |
| Pancreatic ca. PANC-1 | 2.4 |
| Pancreas pool | 0.2 |

Column A - Rel. Exp. (%) Ag7249, Run 318008815

TABLE AC
Panel 4.1D

| Tissue Name | A |
|---|---|
| Secondary Th1 act | 0.0 |
| Secondary Th2 act | 0.0 |
| Secondary Tr1 act | 0.0 |
| Secondary Th1 rest | 0.0 |
| Secondary Th2 rest | 0.0 |
| Secondary Tr1 rest | 0.0 |
| Primary Th1 act | 0.0 |
| Primary Th2 act | 0.0 |
| Primary Tr1 act | 0.0 |
| Primary Th1 rest | 0.0 |
| Primary Th2 rest | 0.0 |
| Primary Tr1 rest | 0.0 |
| CD45RA CD4 lymphocyte act | 0.0 |
| CD45RO CD4 lymphocyte act | 0.0 |
| CD8 lymphocyte act | 0.0 |
| Secondary CD8 lymphocyte rest | 0.0 |
| Secondary CD8 lymphocyte act | 0.0 |
| CD4 lymphocyte none | 0.0 |
| 2ry Th1/Th2/Tr1_anti-CD95 | 0.0 |
| CH11 | |
| LAK cells rest | 0.0 |
| LAK cells IL-2 | 0.0 |
| LAK cells IL-2 + IL-12 | 0.0 |
| LAK cells IL-2 + IFN gamma | 0.0 |
| LAK cells IL-2 + IL-18 | 0.0 |
| LAK cells PMA/ionomycin | 0.0 |
| NK Cells IL-2 rest | 0.0 |
| Two Way MLR 3 day | 0.0 |
| Two Way MLR 5 day | 0.0 |
| Two Way MLR 7 day | 0.0 |
| PBMC rest | 0.0 |
| PBMC PWM | 0.0 |
| PBMC PHA-L | 0.0 |
| Ramos (B cell) none | 49.3 |
| Ramos (B cell) ionomycin | 100.0 |
| B lymphocytes PWM | 0.0 |
| B lymphocytes CD40L and IL-4 | 0.0 |
| EOL-1 dbcAMP | 0.0 |
| EOL-1 dbcAMP PMA/ionomycin | 0.0 |
| Dendritic cells none | 0.0 |
| Dendritic cells LPS | 0.0 |
| Dendritic cells anti-CD40 | 0.0 |
| Monocytes rest | 0.0 |
| Monocytes LPS | 0.0 |
| Macrophages rest | 0.0 |
| Macrophages LPS | 0.0 |
| HUVEC none | 0.0 |
| HUVEC starved | 0.0 |
| HUVEC IL-1beta | 0.0 |
| HUVEC IFN gamma | 0.0 |
| HUVEC TNF alpha + IFN gamma | 0.0 |
| HUVEC TNF alpha + IL4 | 0.0 |
| HUVEC IL-11 | 0.0 |
| Lung Microvascular EC none | 0.0 |
| Lung Microvascular EC TNFalpha + IL-1beta | 0.0 |
| Microvascular Dermal EC none | 0.0 |
| Microsvasular Dermal EC TNFalpha + IL- | 0.0 |
| 1beta | |
| Bronchial epithelium TNFalpha + IL1beta | 0.0 |
| Small airway epithelium none | 0.0 |
| Small airway epithelium TNFalpha + IL-1beta | 0.0 |
| Coronery artery SMC rest | 0.0 |
| Coronery artery SMC TNFalpha + IL-1beta | 0.0 |
| Astrocytes rest | 0.0 |
| Astrocytes TNFalpha + IL-1beta | 0.0 |
| KU-812 (Basophil) rest | 0.0 |
| KU-812 (Basophil) PMA/ionomycin | 0.0 |
| CCD1106 (Keratinocytes) none | 0.0 |
| CCD1106 (Keratinocytes) TNFalpha + IL- | 0.0 |
| 1beta | |
| Liver cirrhosis | 0.0 |
| NCI-H292 none | 0.0 |
| NCI-H292 IL-4 | 0.0 |
| NCI-H292 IL-9 | 0.0 |
| NCI-H292 IL-13 | 0.0 |
| NCI-H292 IFN gamma | 0.0 |
| HPAEC none | 0.0 |
| HPAEC TNF alpha + IL-1 beta | 0.0 |
| Lung fibroblast none | 0.0 |
| Lung fibroblast TNF alpha + IL-1 beta | 0.0 |
| Lung fibroblast IL-4 | 0.0 |
| Lung fibroblast IL-9 | 0.0 |
| Lung fibroblast IL-13 | 0.0 |
| Lung fibroblast IFN gamma | 0.0 |
| Dermal fibroblast CCD1070 rest | 0.0 |
| Dermal fibroblast CCD1070 TNF alpha | 0.0 |
| Dermal fibroblast CCD1070 IL-1 beta | 0.0 |
| Dermal fibroblast IFN gamma | 0.0 |
| Dermal fibroblast IL-4 | 0.0 |
| Dermal Fibroblasts rest | 0.0 |
| Neutrophils TNFa + LPS | 0.0 |
| Neutrophils rest | 0.0 |
| Colon | 0.0 |
| Lung | 0.0 |
| Thymus | 0.0 |
| Kidney | 0.0 |

Column A - Rel. Exp. (%) Ag7249, Run 296433954

General_screening_panel_v1.7 Summary: Ag7249 Highest expression of this gene was detected in ovarian cancer cell line IGROV-1 (CT=28.8) and moderate expression was detected in cancer cell lines derived from melanoma, ovarian, lung, breast, colon and brain cancers. CG103910-02 gene expression is a marker of cancer vs normal tissue and is useful to detect cancers. Therapeutic modulation of this gene, expressed protein and/or the use of antibodies or small molecule drugs targeting this gene or gene product are useful in the treatment of melanoma, ovarian, lung, breast, colon and brain cancers. Gene expression was detected at low levels in certain regions of the central nervous system examined including: amygdala, hippocampus, thalamus, cerebellum, cerebral cortex, and spinal cord. Therapeutic modulation of this gene product is useful in the treatment of central nervous system disorders such as Alzheimer's disease, Parkinson's disease, epilepsy, multiple sclerosis, schizophrenia and depression. Gene expression was detected at low levels in thyroid, fetal heart, kidney, and trachea.

Panel 4.1D Summary: Ag7249 Low expression of this gene was detected in activated Ramos B cells (CT=34.4). Lower but significant gene expression was detected in untreated Ramos B cells. B cells contribute to the immune response through various functional roles, including antibody production and are implicated in the production of auto-antibodies against self-antigens in autoimmune disorders. Therapeutic modulation of this gene, encoded protein and/or antibodies or small molecule drugs that antagonize its function reduce or eliminate the symptoms of patients suffering from asthma, allergies, chronic obstructive pulmonary disease, emphysema, Crohn's disease, ulcerative colitis, rheumatoid arthritis, psoriasis, osteoarthritis, systemic lupus erythematosus and other autoimmune disorders.

B. CG103910-03: BMP7.

Expression of gene CG103910-03 was assessed using the primer-probe set Ag7250, described in Table BA. Results of the RTQ-PCR runs are shown in Tables BB, BC and BD.

TABLE BA

Probe Name Ag7250

| | | | | |
|---|---|---|---|---|
| | | | Start | |
| Primers | Sequences | Length | Position | SEQ ID No |

| | | | | |
|---|---|---|---|---|
| Forward | 5′-acgtggcaggactggatc-3′ | 18 | 950 | 352 |
| Probe | TET-5′-cctgaaggctacgccgcctactactg-3′- | 26 | 974 | 353 |
| | TAMRA | | | |
| Reverse | 5′-gagttcagagggaaggcaca-3′ | 20 | 1010 | 354 |

TABLE BB
AI.05 chondrosarcoma

| Tissue Name | A |
|---|---|
| 138353_PMA (18 hrs) | 11.1 |
| 138352_IL-1beta + Oncostatin M (18 hrs) | 11.7 |
| 138351_IL-1beta + TNFa (18 hrs) | 100.0 |
| 138350_IL-1beta (18 hrs) | 27.5 |
| 138354_Untreated-complete medium | 0.7 |
| (18 hrs) | |
| 138347_PMA (6 hrs) | 22.4 |
| 138346_IL-1beta + Oncostatin M (6 hrs) | 14.6 |
| 138345_IL-1beta + TNFa (6 hrs) | 15.5 |
| 138344_IL-1beta (6 hrs) | 4.7 |
| 138348_Untreated-complete medium | 3.2 |
| (6 hrs) | |
| 138349_Untreated-serum starved (6 hrs) | 10.7 |

Column A - Rel. Exp. (%) Ag7250, Run 312643038

TABLE BC
General_screening_panel_v1.7

| Tissue Name | A |
|---|---|
| Adipose | 1.8 |
| HUVEC | 0.0 |
| Melanoma* Hs688(A).T | 0.0 |
| Melanoma* Hs688(B).T | 9.1 |
| Melanoma (met) SK-MEL-5 | 1.5 |
| Testis | 1.2 |
| Prostate ca. (bone met) PC-3 | 0.0 |
| Prostate ca. DU145 | 0.1 |
| Prostate pool | 1.0 |
| Uterus pool | 0.2 |
| Ovarian ca. OVCAR-3 | 42.6 |
| Ovarian ca. (ascites) SK-OV-3 | 0.0 |
| Ovarian ca. OVCAR-4 | 100.0 |
| Ovarian ca. OVCAR-5 | 0.1 |
| Ovarian ca. IGROV-1 | 38.4 |
| Ovarian ca. OVCAR-8 | 0.0 |
| Ovary | 0.3 |
| Breast ca. MCF-7 | 45.1 |
| Breast ca. MDA-MB-231 | 0.0 |
| Breast ca. BT-549 | 1.2 |
| Breast ca. T47D | 0.1 |
| Breast pool | 0.1 |
| Trachea | 5.4 |
| Lung | 1.4 |
| Fetal Lung | 8.5 |
| Lung ca. NCI-N417 | 0.0 |
| Lung ca. LX-1 | 7.2 |
| Lung ca. NCI-HI 46 | 0.0 |
| Lung ca. SHP-77 | 0.0 |
| Lung ca. NCI-H23 | 10.0 |
| Lung ca. NCI-H460 | 0.1 |
| Lung ca. HOP-62 | 0.0 |
| Lung ca. NCI-H522 | 11.4 |
| Lung ca. DMS-114 | 1.2 |
| Liver | 0.0 |
| Fetal Liver | 0.1 |
| Kidney pool | 5.5 |
| Fetal Kidney | 13.0 |
| Renal ca. 786-0 | 0.0 |
| Renal ca. A498 | 0.0 |
| Renal ca. ACHN | 0.0 |
| Renal ca. UO-31 | 0.0 |
| Renal ca. TK-10 | 0.0 |
| Bladder | 2.4 |
| Gastric ca. (liver met.) NCI-N87 | 0.8 |
| Stomach | 0.1 |
| Colon ca. SW-948 | 26.1 |
| Colon ca. SW480 | 1.0 |
| Colon ca. (SW480 met) SW620 | 38.7 |
| Colon ca. HT29 | 0.0 |
| Colon ca. HCT-116 | 8.4 |
| Colon cancer tissue | 2.4 |
| Colon ca. SW1116 | 7.7 |
| Colon ca. Colo-205 | 10.3 |
| Colon ca. SW-48 | 3.3 |
| Colon | 0.8 |
| Small Intestine | 0.2 |
| Fetal Heart | 4.1 |
| Heart | 0.5 |
| Lymph Node pool 1 | 0.7 |
| Lymph Node pool 2 | 0.9 |
| Fetal Skeletal Muscle | 0.9 |
| Skeletal Muscle pool | 0.0 |
| Skeletal Muscle | 0.2 |
| Spleen | 0.4 |
| Thymus | 1.0 |
| CNS cancer (glio/astro) SF-268 | 0.0 |
| CNS cancer (glio/astro) T98G | 4.9 |
| CNS cancer (neuro; met) SK-N-AS | 0.1 |
| CNS cancer (astro) SF-539 | 0.0 |
| CNS cancer (astro) SNB-75 | 1.6 |
| CNS cancer (glio) SNB-19 | 0.2 |
| CNS cancer (glio) SF-295 | 7.9 |
| Brain (Amygdala) | 6.9 |
| Brain (Cerebellum) | 6.4 |
| Brain (Fetal) | 9.0 |
| Brain (Hippocampus) | 11.1 |
| Cerebral Cortex pool | 5.4 |
| Brain (Substantia nigra) | 2.6 |
| Brain (Thalamus) | 7.6 |
| Brain (Whole) | 19.3 |
| Spinal Cord | 6.2 |
| Adrenal Gland | 2.1 |
| Pituitary Gland | 0.7 |
| Salivary Gland | 0.2 |
| Thyroid | 60.3 |
| Pancreatic ca. PANC-1 | 2.4 |
| Pancreas pool | 0.5 |

Column A - Rel. Exp. (%) Ag7250, Run 318008817

TABLE BD
Panel 4.1D

| Tissue Name | A |
|---|---|
| Secondary Th1 act | 0.1 |
| Secondary Th2 act | 0.1 |
| Secondary Tr1 act | 0.0 |
| Secondary Th1 rest | 0.0 |
| Secondary Th2 rest | 0.0 |
| Secondary Tr1 rest | 0.0 |
| Primary Th1 act | 0.1 |
| Primary Th2 act | 0.1 |
| Primary Tr1 act | 0.0 |
| Primary Th1 rest | 0.0 |
| Primary Th2 rest | 0.0 |
| Primary Tr1 rest | 0.0 |
| CD45RA CD4 lymphocyte act | 0.0 |
| CD45RO CD4 lymphocyte act | 0.2 |
| CD8 lymphocyte act | 0.0 |
| Secondary CD8 lymphocyte rest | 0.0 |
| Secondary CD8 lymphocyte act | 0.0 |
| CD4 lymphocyte none | 0.0 |
| 2ry Th1/Th2/Tr1 anti-CD95 | 0.0 |
| CH11 | |
| LAK cells rest | 0.0 |
| LAK cells IL-2 | 0.0 |
| LAK cells IL-2 + IL-12 | 0.0 |
| LAK cells IL-2 + IFN gamma | 0.1 |
| LAK cells IL-2 + IL-18 | 0.0 |
| LAK cells PMA/ionomycin | 0.0 |
| NK Cells IL-2 rest | 0.0 |
| Two Way MLR 3 day | 0.0 |
| Two Way MLR 5 day | 0.0 |
| Two Way MLR 7 day | 0.0 |
| PBMC rest | 0.0 |
| PBMC PWM | 0.0 |
| PBMC PHA-L | 0.0 |
| Ramos (B cell) none | 38.4 |
| Ramos (B cell) ionomycin | 100.0 |
| B lymphocytes PWM | 0.1 |
| B lymphocytes CD40L and IL-4 | 1.5 |
| EOL-1 dbcAMP | 0.0 |
| EOL-1 dbcAMP PMA/ionomycin | 0.0 |
| Dendritic cells none | 0.0 |
| Dendritic cells LPS | 0.0 |
| Dendritic cells anti-CD40 | 0.0 |
| Monocytes rest | 0.0 |
| Monocytes LPS | 0.0 |
| Macrophages rest | 0.1 |
| Macrophages LPS | 0.0 |
| HUVEC none | 0.0 |
| HUVEC starved | 0.0 |
| HUVEC IL-1beta | 0.0 |
| HUVEC IFN gamma | 0.0 |
| HUVEC TNF alpha + IFN gamma | 0.0 |
| HUVEC TNF alpha + IL4 | 0.0 |
| HUVEC IL-11 | 0.0 |
| Lung Microvascular EC none | 0.0 |
| Lung Microvascular EC TNFalpha + IL-1beta | 0.0 |
| Microvascular Dermal EC none | 0.0 |
| Microsvasular Dermal EC TNFalpha + IL- | 0.0 |
| 1beta | |
| Bronchial epithelium TNFalpha + IL1beta | 0.1 |
| Small airway epithelium none | 0.0 |
| Small airway epithelium TNFalpha + IL-1beta | 0.3 |
| Coronery artery SMC rest | 0.0 |
| Coronery artery SMC TNFalpha + IL-1beta | 0.0 |
| Astrocytes rest | 0.3 |
| Astrocytes TNFalpha + IL-1beta | 0.0 |
| KU-812 (Basophil) rest | 0.0 |
| KU-812 (Basophil) PMA/ionomycin | 0.0 |
| CCD1106 (Keratinocytes) none | 0.1 |
| CCD1106 (Keratinocytes) TNFalpha + IL- | 0.0 |
| 1beta | |
| Liver cirrhosis | 0.2 |
| NCI-H292 none | 0.5 |
| NCI-H292 IL-4 | 0.0 |
| NCI-H292 IL-9 | 0.4 |
| NCI-H292 IL-13 | 0.2 |
| NCI-H292 IFN gamma | 0.3 |
| HPAEC none | 0.0 |
| HPAEC TNF alpha + IL-1 beta | 0.0 |
| Lung fibroblast none | 0.0 |
| Lung fibroblast TNF alpha + IL-1 beta | 0.0 |
| Lung fibroblast IL-4 | 0.0 |
| Lung fibroblast IL-9 | 0.0 |
| Lung fibroblast IL- 1 3 | 0.0 |
| Lung fibroblast IFN gamma | 0.1 |
| Dermal fibroblast CCD1070 rest | 0.0 |
| Dermal fibroblast CCD1070 TNF alpha | 0.0 |
| Dermal fibroblast CCD1070 IL-1 beta | 0.0 |
| Dermal fibroblast IFN gamma | 0.1 |
| Dermal fibroblast IL-4 | 0.0 |
| Dermal Fibroblasts rest | 0.9 |
| Neutrophils TNFa + LPS | 0.0 |
| Neutrophils rest | 0.0 |
| Colon | 0.2 |
| Lung | 0.1 |
| Thymus | 1.6 |
| Kidney | 3.5 |

Column A - Rel. Exp. (%) Ag7250, Run 296422907

AI.05 chondrosarcoma Summary: Ag7250 Highest CG103910-03 gene expression was detected in activated chondrosarcoma cell line (CT=28.4). Gene expression was upregulated upon IL-1 treatment, a potent activator of pro-inflammatory cytokines and matrix metalloproteinases which participate in the destruction of cartilage observed in Osteoarthritis (OA). Modulation of gene expression or its encoded protein and/or the use of antibodies, small molecules or antisense targeting the gene or the encoded protein are important for preventing the degeneration of cartilage observed in OA.

General_screening_panel_v1.7 Summary: Ag7250 Highest gene expression was detected in ovarian cancer OVCAR cell line (CT=22.6). High gene expression was detected in cancer cell lines derived from melanoma, ovarian, lung, breast, colon and brain cancers. CG103910-03 gene expression is a marker useful to differentiate these cancers and to detect their presence in vitro and in vivo. Therapeutic modulation of this gene or encoded protein and/or use of antibodies or small molecule drug targeting the gene or the encoded protein is useful in the treatment of melanoma, ovarian, lung, breast, colon and brain cancers. Gene expression was high in all the regions of the central nervous system examined including: amygdala, hippocampus, thalamus, cerebellum, substantia nigra, cerebral cortex, and spinal cord. Therapeutic modulation of this gene or the encoded protein is useful in the treatment of central nervous system disorders such as Alzbeimer's disease, Parkinson's disease, epilepsy, multiple sclerosis, schizophrenia and depression. Among tissues with metabolic or endocrine function, gene expression was moderate in pancreas, adipose, adrenal gland, thyroid, pituitary gland, skeletal muscle, heart, fetal liver and the gastrointestinal tract. Therapeutic modulation of the activity of this gene is useful in the treatment of endocrine/metabolically related diseases, such as obesity and diabetes.

Panel 4.1D Summary: Ag7250 Highest gene expression was detected in activated Ramos B cells (CT=28.4) with significant expression also detected in untreated Ramos B cells. B cells contribute to the immune response through various functional roles, including antibody production and are implicated in the production of auto-antibodies against self-antigens in autoimmune disorders. Therapeutic modulation of this gene, encoded protein and/or antibodies or small molecule drugs that antagonize its function reduce or eliminate the symptoms of patients suffering from asthma, allergies, chronic obstructive pulmonary disease, emphysema, Crohn's disease, ulcerative colitis, rheumatoid arthritis, psoriasis, osteoarthritis, systemic lupus erythematosus and other autoimmune disorders.

C. CG183860-01: Novel Membrane Protein.

Expression of gene CG183860-01 was assessed using the primer-probe set Ag6837, described in Table CA. Results of the RTQ-PCR runs are shown in Tables CB, CC and CD. Table CA. Probe Name Ag6837

TABLEca

Probe Name Ag7249

| | | | | |
|---|---|---|---|---|
| | | | | SEQ ID |
| Primers | Sequences | Length | Start Position | No |

| | | | | |
|---|---|---|---|---|
| Forward | 5′-ggctgagccaggtggat-3′ | 17 | 1168 | 355 |
| Probe | TET-5′-cgccatacaccaccagccactca-3′- | 23 | 1185 | 356 |
| | TAMRA | | | |
| Reverse | 5′-catccgcaggttctcctt-3′ | 18 | 1249 | 357 |

TABLE CB
CNS_neurodegeneration_v1.0

| Tissue Name | A |
|---|---|
| AD 1 Hippo | 0.2 |
| AD 2 Hippo | 3.3 |
| AD 3 Hippo | 12.7 |
| AD 4 Hippo | 3.7 |
| AD 5 hippo | 36.9 |
| AD 6 Hippo | 100.0 |
| Control 2 Hippo | 5.2 |
| Control 4 Hippo | 1.8 |
| Control (Path) 3 Hippo | 4.7 |
| AD 1 Temporal Ctx | 6.4 |
| AD 2 Temporal Ctx | 10.8 |
| AD 3 Temporal Ctx | 2.1 |
| AD 4 Temporal Ctx | 9.9 |
| AD 5 Inf Temporal Ctx | 9.5 |
| AD 5 SupTemporal Ctx | 25.3 |
| AD 6 Inf Temporal Ctx | 92.7 |
| AD 6 Sup Temporal Ctx | 81.2 |
| Control 1 Temporal Ctx | 7.2 |
| Control 2 Temporal Ctx | 9.7 |
| Control 3 Temporal Ctx | 9.3 |
| Control 4 Temporal Ctx | 5.1 |
| Control (Path) 1 Temporal Ctx | 5.0 |
| Control (Path) 2 Temporal Ctx | 1.9 |
| Control (Path) 3 Temporal Ctx | 12.4 |
| Control (Path) 4 Temporal Ctx | 12.2 |
| AD 1 Occipital Ctx | 15.6 |
| AD 2 Occipital Ctx (Missing) | 0.0 |
| AD 3 Occipital Ctx | 19.1 |
| AD 4 Occipital Ctx | 3.3 |
| AD 5 Occipital Ctx | 15.8 |
| AD 6 Occipital Ctx | 0.0 |
| Control 1 Occipital Ctx | 11.7 |
| Control 2 Occipital Ctx | 9.3 |
| Control 3 Occipital Ctx | 7.9 |
| Control 4 Occipital Ctx | 5.4 |
| Control (Path) 1 Occipital Ctx | 19.9 |
| Control (Path) 2 Occipital Ctx | 5.4 |
| Control (Path) 3 Occipital Ctx | 8.2 |
| Control (Path) 4 Occipital Ctx | 21.6 |
| Control 1 Parietal Ctx | 7.6 |
| Control 2 Parietal Ctx | 33.9 |
| Control 3 Parietal Ctx | 6.1 |
| Control (Path) 1 Parietal Ctx | 10.8 |
| Control (Path) 2 Parietal Ctx | 4.0 |
| Control (Path) 3 Parietal Ctx | 13.2 |
| Control (Path) 4 Parietal Ctx | 9.9 |

Column A - Rel. Exp. (%) Ag6837, Run 279057825

TABLE CC
General_screening_panel_v1.6

| Tissue Name | A |
|---|---|
| Adipose | 0.4 |
| Melanoma* Hs688(A).T | 0.0 |
| Melanoma* Hs688(B).T | 0.0 |
| Melanoma* M14 | 0.0 |
| Melanoma* LOXIMVI | 0.0 |
| Melanoma* SK-MEL-5 | 0.0 |
| Squamous cell carcinoma SCC-4 | 0.0 |
| Testis Pool | 0.7 |
| Prostate ca.* (bone met) PC-3 | 100.0 |
| Prostate Pool | 0.2 |
| Placenta | 0.0 |
| Uterus Pool | 0.1 |
| Ovarian ca. OVCAR-3 | 0.0 |
| Ovarian ca. SK-OV-3 | 0.2 |
| Ovarian ca. OVCAR-4 | 0.0 |
| Ovarian ca. OVCAR-5 | 0.4 |
| Ovarian ca. IGROV-1 | 11.2 |
| Ovarian ca. OVCAR-8 | 0.0 |
| Ovary | 0.0 |
| Breast ca. MCF-7 | 0.0 |
| Breast ca. MDA-MB-231 | 0.3 |
| Breast ca. BT 549 | 0.1 |
| Breast ca. T47D | 0.0 |
| Breast ca. MDA-N | 0.0 |
| Breast Pool | 0.1 |
| Trachea | 1.1 |
| Lung | 0.4 |
| Fetal Lung | 4.6 |
| Lung ca. NCI-N417 | 0.0 |
| Lung ca. LX-1 | 0.0 |
| Lung ca. NCI-H146 | 0.0 |
| Lung ca. SHP-77 | 0.0 |
| Lung ca. A549 | 0.0 |
| Lung ca. NCI-H526 | 0.0 |
| Lung ca. NCI-H23 | 0.2 |
| Lung ca. NCI-H460 | 0.4 |
| Lung ca. HOP-62 | 0.2 |
| Lung ca. NCI-H522 | 0.0 |
| Liver | 0.1 |
| Fetal Liver | 0.3 |
| Liver ca. HepG2 | 0.0 |
| Kidney Pool | 0.2 |
| Fetal Kidney | 16.6 |
| Renal ca. 786-0 | 0.0 |
| Renal ca. A498 | 0.1 |
| Renal ca. ACHN | 0.0 |
| Renal ca. UO-31 | 0.1 |
| Renal ca. TK-10 | 0.0 |
| Bladder | 0.1 |
| Gastric ca. (liver met.) NCI-N87 | 0.1 |
| Gastric ca. KATO III | 0.0 |
| Colon ca. SW-948 | 0.0 |
| Colon ca. SW480 | 0.0 |
| Colon ca.* (SW480 met) SW620 | 0.0 |
| Colon ca. HT29 | 0.0 |
| Colon ca. HCT-116 | 0.0 |
| Colon ca. CaCo-2 | 0.0 |
| Colon cancer tissue | 0.1 |
| Colon ca. SW1116 | 0.0 |
| Colon ca. Colo-205 | 0.0 |
| Colon ca. SW-48 | 0.0 |
| Colon Pool | 0.1 |
| Small Intestine Pool | 1.7 |
| Stomach Pool | 0.3 |
| Bone Marrow Pool | 0.1 |
| Fetal Heart | 0.9 |
| Heart Pool | 0.5 |
| Lymph Node Pool | 0.2 |
| Fetal Skeletal Muscle | 0.6 |
| Skeletal Muscle Pool | 0.0 |
| Spleen Pool | 0.1 |
| Thymus Pool | 0.4 |
| CNS cancer (glio/astro) U87-MG | 0.0 |
| CNS cancer (glio/astro) U-118-MG | 0.0 |
| CNS cancer (neuro; met) SK-N-AS | 1.4 |
| CNS cancer (astro) SF-539 | 0.0 |
| CNS cancer (astro) SNB-75 | 0.1 |
| CNS cancer (glio)SNB- 19 | 10.4 |
| CNS cancer (glio) SF-295 | 0.5 |
| Brain (Amygdala) Pool | 0.1 |
| Brain (cerebellum) | 0.1 |
| Brain (fetal) | 0.1 |
| Brain (Hippocampus) Pool | 0.1 |
| Cerebral Cortex Pool | 0.0 |
| Brain (Substantia nigra) Pool | 0.0 |
| Brain (Thalamus) Pool | 0.1 |
| Brain (whole) | 0.1 |
| Spinal Cord Pool | 0.1 |
| Adrenal Gland | 0.6 |
| Pituitary gland Pool | 0.1 |
| Salivary Gland | 0.1 |
| Thyroid (female) | 0.2 |
| Pancreatic ca. CAPAN2 | 0.1 |
| Pancreas Pool | 0.0 |

Column A - Rel. Exp. (%) Ag6837, Run 278368620

TABLE CD
Panel 4.1D

| Tissue Name | A |
|---|---|
| Secondary Th1 act | 0.0 |
| Secondary Th2 act | 1.1 |
| Secondary Tr1 act | 0.0 |
| Secondary Th1 rest | 0.0 |
| Secondary Th2 rest | 0.0 |
| Secondary Tr1 rest | 0.0 |
| Primary Th1 act | 0.0 |
| Primary Th2 act | 1.8 |
| Primary Tr1 act | 0.0 |
| Primary Th1 rest | 0.0 |
| Primary Th2 rest | 0.0 |
| Primary Tr1 rest | 0.0 |
| CD45RA CD4 lymphocyte act | 0.0 |
| CD45RO CD4 lymphocyte act | 0.0 |
| CD8 lymphocyte act | 0.0 |
| Secondary CD8 lymphocyte rest | 0.0 |
| Secondary CD8 lymphocyte act | 1.7 |
| CD4 lymphocyte none | 0.0 |
| 2ry Th1/Th2/Tr1_anti-CD95 | 0.0 |
| CH11 | |
| LAK cells rest | 1.4 |
| LAK cells IL-2 | 1.6 |
| LAK cells IL-2 + IL-12 | 0.0 |
| LAK cells IL-2 + IFN gamma | 0.0 |
| LAK cells IL-2 + IL-18 | 0.0 |
| LAK cells PMA/ionomycin | 0.0 |
| NK Cells IL-2 rest | 0.0 |
| Two Way MLR 3 day | 0.0 |
| Two Way MLR 5 day | 0.0 |
| Two Way MLR 7 day | 0.0 |
| PBMC rest | 0.0 |
| PBMC PWM | 0.0 |
| PBMC PHA-L | 0.0 |
| Ramos (B cell) none | 0.0 |
| Ramos (B cell) ionomycin | 0.0 |
| B lymphocytes PWM | 0.0 |
| B lymphocytes CD40L and IL-4 | 2.1 |
| EOL-1 dbcAMP | 0.0 |
| EOL-1 dbcAMP PMA/ionomycin | 0.0 |
| Dendritic cells none | 0.0 |
| Dendritic cells LPS | 0.0 |
| Dendritic cells anti-CD40 | 0.0 |
| Monocytes rest | 0.0 |
| Monocytes LPS | 0.0 |
| Macrophages rest | 0.0 |
| Macrophages LPS | 0.0 |
| HUVEC none | 57.0 |
| HUVEC starved | 42.9 |
| HUVEC IL-1beta | 33.7 |
| HUVEC IFN gamma | 28.9 |
| HUVEC TNF alpha + IFN gamma | 14.2 |
| HUVEC TNF alpha + IL4 | 16.0 |
| HUVEC IL-11 | 25.9 |
| Lung Microvascular EC none | 7.1 |
| Lung Microvascular EC TNFalpha + IL-1beta | 1.7 |
| Microvascular Dermal EC none | 5.6 |
| Microsvasular Dermal EC TNFalpha + IL- | 0.0 |
| 1beta | |
| Bronchial epithelium TNFalpha + IL1beta | 0.0 |
| Small airway epithelium none | 0.0 |
| Small airway epithelium TNFalpha + IL-1beta | 0.0 |
| Coronery artery SMC rest | 3.4 |
| Coronery artery SMC TNFalpha + IL-1beta | 2.7 |
| Astrocytes rest | 0.0 |
| Astrocytes TNFalpha + IL-1beta | 0.0 |
| KU-812 (Basophil) rest | 0.0 |
| KU-812 (Basophil) PMA/ionomycin | 0.0 |
| CCD1106 (Keratinocytes) none | 0.0 |
| CCD1106 (Keratinocytes) TNFalpha + IL- | 0.0 |
| 1beta | |
| Liver cirrhosis | 6.9 |
| NCI-H292 none | 0.0 |
| NCI-H292 IL-4 | 0.0 |
| NCI-H292 IL-9 | 0.0 |
| NCI-H292 IL-13 | 0.0 |
| NCI-H292 IFN gamma | 0.0 |
| HPAEC none | 49.3 |
| HPAEC TNF alpha + IL-1 beta | 13.4 |
| Lung fibroblast none | 3.0 |
| Lung fibroblast TNF alpha + IL-1 beta | 0.0 |
| Lung fibroblast IL-4 | 1.2 |
| Lung fibroblast IL-9 | 0.0 |
| Lung fibroblast IL-13 | 2.0 |
| Lung fibroblast IFN gamma | 0.0 |
| Dermal fibroblast CCD1070 rest | 0.0 |
| Dermal fibroblast CCD1070 TNF alpha | 0.0 |
| Dermal fibroblast CCD1070 IL-1 beta | 0.0 |
| Dermal fibroblast IFN gamma | 0.0 |
| Dermal fibroblast IL-4 | 2.2 |
| Dermal Fibroblasts rest | 0.0 |
| Neutrophils TNFa + LPS | 0.0 |
| Neutrophils rest | 0.0 |
| Colon | 2.8 |
| Lung | 3.7 |
| Thymus | 0.0 |
| Kidney | 100.0 |

Column A - Rel. Exp. (%) Ag6837, Run 279029111

CNS_neurodegeneration_v1.0 Summary: Ag6837 Highest CG183860 gene expression was detected in the hippocampus of an Alzheimer's patient (CT=31) and was also determined to be upregulated in the temporal cortex of Alzheimer's disease patients. Therapeutic modulation of the expression or function of this gene, encoded protein and/or use of antibodies or small molecule drug targeting the encoded protein to decrease neuronal cell death is useful in the treatment of this disease.

General_screening_panel_v1.6 Summary: Ag6837 Highest gene expression was detected in a prostate cancer cell line (CT=24.5) and high levels of expression were also seen in ovarian and brain cancer cell lines. CG183860 gene expression is a marker for differentiating cancerous from normal tissues and to detect the presence of these cancers. Therapeutic modulation of the expression or function of this gene, encoded protein and/or use of antibodies or small molecule drug targeting the encoded protein are effective in the treatment of cancer. Gene expression was also detected at higher levels in fetal kidney and lung (CTs=27-29) relative to expression in the corresponding adult tissues (CTs=32-33). The relative over expression of this gene in these fetal tissues suggests that the protein product may enhance lung and kidney growth or development in the fetus and are useful in a regenerative capacity in the adult.

Panel 4.1D Summary: Ag6837 Highest expression was seen in kidney (CT-32.7). Low but significant gene expression was detected in samples derived from human endothelium cells from umbilical vein and pulmonary artery (HUVEC and HPAEC). Therapeutic modulation of this gene, encoded protein and/or antibodies, small molecule drug targeting the encoded protein will reduce or eliminate the symptoms in patients with autoimmune and inflammatory diseases that involve endothelial cells, such as lupus erythematosus, asthma, emphysema, Crohn's disease, ulcerative colitis, rheumatoid arthritis, osteoarthritis, and psoriasis.

D. CG10590-02: Similar to Chordin-Like.

Expression of gene CG110590-02 was assessed using the primer-probe set Ag7182, described in Table DA. Results of the RTQ-PCR runs are shown in Table DB.

Table DA. Probe Name Ag7182

TABLE DA

Probe Name Ag7249

| | | | | |
|---|---|---|---|---|
| | | | Start | SEQ ID |
| Primers | Sequences | Length | Position | No |

| | | | | |
|---|---|---|---|---|
| Forward | 5′-gaccacctcaggcattctc-3′ | 19 | 1220 | 358 |
| Probe | TET-5′-ctcctcaaacatcctcttggagatcttct-3′- | 29 | 1255 | 359 |
| | TAMRA | | | |
| Reverse | 5′-ctcagggttgttctggtcac-3′ | 20 | 1302 | 360 |

TABLE DB
General_screening_panel_v1.7

| Tissue Name | A |
|---|---|
| Adipose | 100.0 |
| HUVEC | 0.0 |
| Melanoma* Hs688(A).T | 0.0 |
| Melanoma* Hs688(B).T | 0.0 |
| Melanoma (met) SK-MEL-5 | 0.0 |
| Testis | 6.2 |
| Prostate ca. (bone met) PC-3 | 0.0 |
| Prostate ca. DU145 | 0.0 |
| Prostate pool | 11.0 |
| Uterus pool | 3.4 |
| Ovarian ca. OVCAR-3 | 0.9 |
| Ovarian ca. (ascites) SK-OV-3 | 1.5 |
| Ovarian ca. OVCAR-4 | 0.0 |
| Ovarian ca. OVCAR-5 | 2.1 |
| Ovarian ca. IGROV-1 | 0.0 |
| Ovarian ca. OVCAR-8 | 14.7 |
| Ovary | 35.1 |
| Breast ca. MCF-7 | 0.0 |
| Breast ca. MDA-MB-231 | 0.4 |
| Breast ca. BT-549 | 3.6 |
| Breast ca. T47D | 0.0 |
| Breast pool | 0.0 |
| Trachea | 15.3 |
| Lung | 54.3 |
| Fetal Lung | 12.2 |
| Lung ca. NCI-N417 | 0.0 |
| Lung ca. LX-1 | 0.0 |
| Lung ca. NCI-H146 | 0.0 |
| Lung ca. SHP-77 | 0.4 |
| Lung ca. NCI-H23 | 0.4 |
| Lung ca. NCI-H460 | 0.0 |
| Lung ca. HOP-62 | 0.0 |
| Lung ca. NCI-H522 | 0.0 |
| Lung ca. DMS-114 | 0.0 |
| Liver | 0.0 |
| Fetal Liver | 0.0 |
| Kidney pool | 7.9 |
| Fetal Kidney | 0.9 |
| Renal ca. 786-0 | 0.0 |
| Renal ca. A498 | 0.0 |
| Renal ca. ACHN | 7.2 |
| Renal ca. UO-31 | 0.0 |
| Renal ca. TK-10 | 0.0 |
| Bladder | 9.4 |
| Gastric ca. (liver met.) NCI-N87 | 0.0 |
| Stomach | 0.7 |
| Colon ca. SW-948 | 0.0 |
| Colon ca. SW480 | 0.0 |
| Colon ca. (SW480 met) SW620 | 0.0 |
| Colon ca. HT29 | 0.0 |
| Colon ca. HCT-116 | 0.0 |
| Colon cancer tissue | 0.0 |
| Colon ca. SW1116 | 0.0 |
| Colon ca. Colo-205 | 0.0 |
| Colon ca. SW-48 | 0.0 |
| Colon | 6.2 |
| Small Intestine | 3.4 |
| Fetal Heart | 0.0 |
| Heart | 15.9 |
| Lymph Node pool 1 | 0.0 |
| Lymph Node pool 2 | 17.7 |
| Fetal Skeletal Muscle | 1.0 |
| Skeletal Muscle pool | 0.8 |
| Skeletal Muscle | 1.4 |
| Spleen | 3.6 |
| Thymus | 1.6 |
| CNS cancer (glio/astro) SF-268 | 1.0 |
| CNS cancer (glio/astro) T98G | 0.0 |
| CNS cancer (neuro; met) SK-N-AS | 0.0 |
| CNS cancer (astro) SF-539 | 1.7 |
| CNS cancer (astro) SNB-75 | 40.6 |
| CNS cancer (glio) SNB-19 | 0.0 |
| CNS cancer (glio) SF-295 | 0.0 |
| Brain (Amygdala) | 4.0 |
| Brain (Cerebellum) | 6.7 |
| Brain (Fetal) | 19.3 |
| Brain (Hippocampus) | 6.9 |
| Cerebral Cortex pool | 8.2 |
| Brain (Substantia nigra) | 3.0 |
| Brain (Thalamus) | 5.3 |
| Brain (Whole) | 33.4 |
| Spinal Cord | 0.0 |
| Adrenal Gland | 2.9 |
| Pituitary Gland | 4.0 |
| Salivary Gland | 1.8 |
| Thyroid | 14.5 |
| Pancreatic ca. PANC-1 | 0.0 |
| Pancreas pool | 0.4 |

Column A - Rel. Exp. (%) Ag7182, Run 318350049

General_screening_panel_v1.7 Summary: Ag7182 Highest CG110590 gene expression was seen in adipose (CT=31.9). Therapeutic modulation of this gene and encoded protein is useful in the treatment of adipose related diseases such as obesity and diabetes. Low gene expression was seen in fetal and adult brain. Therapeutic modulation of this gene and/or encoded protein is useful in the treatment of neurological disorders such as Alzheimer's disease, Parkinson's disease, epilepsy, multiple sclerosis, schizophrenia and depression.

E. CG184416-01: Metalloprotease mmp21/22C.

Expression of gene CG 184416-01 was assessed using the primer-probe set Ag7014, described in Table EA. Results of the RTQ-PCR runs are shown in Table EB.

Table EA. Probe Name Ag7014

TABLE EA

Probe Name Ag7014

| | | | | |
|---|---|---|---|---|
| Primers | Sequences | Length | Start Position | SEQ ID No |

| | | | | |
|---|---|---|---|---|
| Forward | 5′-cgctcccgataggatgc-3′ | 17 | 862 | 361 |
| Probe | TET-5′-acgcgcacacgaacagcctgtc-3′-TAMRA | 22 | 882 | 362 |
| Reverse | 5′-ggcagagcctcttcatgag-3′ | 19 | 942 | 363 |

TABLE EB
General_screening_panel_v1.6

| Tissue Name | A |
|---|---|
| Adipose | 0.3 |
| Melanoma* Hs688(A).T | 0.0 |
| Melanoma* Hs688(B).T | 1.5 |
| Melanoma* M14 | 0.0 |
| Melanoma* LOXIMVI | 0.0 |
| Melanoma* SK-MEL-5 | 1.5 |
| Squamous cell carcinoma SCC-4 | 1.6 |
| Testis Pool | 0.0 |
| Prostate ca.* (bone met) PC-3 | 9.5 |
| Prostate Pool | 0.0 |
| Placenta | 0.8 |
| Uterus Pool | 1.9 |
| Ovarian ca. OVCAR-3 | 0.0 |
| Ovarian ca. SK-OV-3 | 0.9 |
| Ovarian ca. OVCAR-4 | 0.3 |
| Ovarian ca. OVCAR-5 | 2.7 |
| Ovarian ca. IGROV-1 | 0.0 |
| Ovarian ca. OVCAR-8 | 100.0 |
| Ovary | 0.0 |
| Breast ca. MCF-7 | 1.3 |
| Breast ca. MDA-MB-231 | 1.2 |
| Breast ca. BT 549 | 3.3 |
| Breast ca. T47D | 0.0 |
| Breast ca. MDA-N | 0.6 |
| Breast Pool | 4.7 |
| Trachea | 1.2 |
| Lung | 0.0 |
| Fetal Lung | 4.7 |
| Lung ca. NCI-N417 | 1.8 |
| Lung ca. LX-1 | 0.7 |
| Lung ca. NCI-H146 | 0.5 |
| Lung ca. SHP-77 | 1.1 |
| Lung ca. A549 | 0.0 |
| Lung ca. NCI-H526 | 0.0 |
| Lung ca. NCI-H23 | 0.0 |
| Lung ca. NCI-H460 | 0.0 |
| Lung ca. HOP-62 | 2.3 |
| Lung ca. NCI-H522 | 0.0 |
| Liver | 0.0 |
| Fetal Liver | 1.0 |
| Liver ca. HepG2 | 0.0 |
| Kidney Pool | 7.1 |
| Fetal Kidney | 0.0 |
| Renal ca. 786-0 | 1.6 |
| Renal ca. A498 | 0.0 |
| Renal ca. ACHN | 0.0 |
| Renal ca. UO-31 | 0.0 |
| Renal ca. TK-10 | 1.3 |
| Bladder | 2.5 |
| Gastric ca. (liver met.) NCI-N87 | 0.0 |
| Gastric ca. KATO III | 1.6 |
| Colon ca. SW-948 | 0.0 |
| Colon ca. SW480 | 0.0 |
| Colon ca.* (SW480 met) SW620 | 0.9 |
| Colon ca. HT29 | 0.0 |
| Colon ca. HCT-116 | 1.4 |
| Colon ca. CaCo-2 | 0.0 |
| Colon cancer tissue | 2.0 |
| Colon ca. SW1116 | 4.9 |
| Colon ca. Colo-205 | 0.0 |
| Colon ca. SW-48 | 1.2 |
| Colon Pool | 1.1 |
| Small Intestine Pool | 0.2 |
| Stomach Pool | 0.6 |
| Bone Marrow Pool | 1.1 |
| Fetal Heart | 2.8 |
| Heart Pool | 2.6 |
| Lymph Node Pool | 6.1 |
| Fetal Skeletal Muscle | 0.0 |
| Skeletal Muscle Pool | 0.8 |
| Spleen Pool | 0.2 |
| Thymus Pool | 0.1 |
| CNS cancer (glio/astro) U87-MG | 0.0 |
| CNS cancer (glio/astro) U-118-MG | 0.0 |
| CNS cancer (neuro; met) SK-N-AS | 0.0 |
| CNS cancer (astro) SF-539 | 0.5 |
| CNS cancer (astro) SNB-75 | 5.1 |
| CNS cancer (glio) SNB-19 | 2.2 |
| CNS cancer (glio) SF-295 | 1.1 |
| Brain (Amygdala) Pool | 0.0 |
| Brain (cerebellum) | 1.0 |
| Brain (fetal) | 0.0 |
| Brain (Hippocampus) Pool | 1.1 |
| Cerebral Cortex Pool | 0.0 |
| Brain (Substantia nigra) Pool | 0.0 |
| Brain (Thalamus) Pool | 0.0 |
| Brain (whole) | 0.0 |
| Spinal Cord Pool | 0.0 |
| Adrenal Gland | 0.0 |
| Pituitary gland Pool | 2.0 |
| Salivary Gland | 0.0 |
| Thyroid (female) | 0.0 |
| Pancreatic ca. CAPAN2 | 0.1 |
| Pancreas Pool | 1.5 |

Column A - Rel. Exp. (%) Ag7014, Run 279032748

General_screening_panel_v1.6 Summary: Ag7014 Highest gene expression was detected in an ovarian cancer cell line (CT=29.8), with low but significant expression in a prostate cancer cell line. Gene expression level is a marker of ovarian and prostate cancer tissue and for detecting the presence of these cancers in vitro or in vivo.

F. CG50513-01: Tumor-Related Protein (PDRC1).

Expression of gene CG50513-01 was assessed using the primer-probe sets Ag2752 and Ag5, described in Tables FA and FB. Results of the RTQ-PCR runs are shown in Tables FC, FD, FE and FF.

TABLE FA

Probe Name Ag2752

| | | | | |
|---|---|---|---|---|
| | | | Start | |
| Primers | Sequences | Length | Position | SEQ ID No |

| | | | | |
|---|---|---|---|---|
| Forward | 5′-gaagacagctggagagagtttg-3′ | 22 | 1387 | 364 |
| Probe | TET-5′-cttgtcctgcatggccaatccagt-3′- | 24 | 1410 | 365 |
| | TAMRA | | | |
| Reverse | 5′-agctgcataatgaagagctgat-3′ | 22 | 1450 | 366 |

TABLE FB

Probe Name Ag5

| | | | | |
|---|---|---|---|---|
| | | | Start | |
| Primers | Sequences | Length | Position | SEQ ID No |

| | | | | |
|---|---|---|---|---|
| Forward | 5′-gtgatcctcaggctggacca-3′ | 20 | 1219 | 367 |
| Probe | TET-5′-ccagtgtttcctcagcacagggcc-3′- | 24 | 1253 | 368 |
| | TAMRA | | | |
| Reverse | 5′-ttctgactgggctgcatcc-3′ | 19 | 1278 | 369 |

TABLE FC
Panel 1

| Tissue Name | A |
|---|---|
| Endothelial cells | 0.0 |
| Endothelial cells (treated) | 0.0 |
| Pancreas | 0.0 |
| Pancreatic ca. CAPAN 2 | 0.0 |
| Adrenal gland | 0.2 |
| Thyroid | 3.5 |
| Salivary gland | 33.7 |
| Pituitary gland | 1.6 |
| Brain (fetal) | 0.0 |
| Brain (whole) | 0.0 |
| Brain (amygdala) | 0.0 |
| Brain (cerebellum) | 0.2 |
| Brain (hippocampus) | 0.1 |
| Brain (substantia nigra) | 0.0 |
| Brain (thalamus) | 0.0 |
| Brain (hypothalamus) | 0.0 |
| Spinal cord | 1.7 |
| glio/astro U87-MG | 0.0 |
| glio/astro U-118-MG | 0.0 |
| astrocytoma SW1783 | 0.0 |
| neuro*; met SK-N-AS | 0.2 |
| astrocytoma SF-539 | 0.0 |
| astrocytoma SNB-75 | 0.0 |
| glioma SNB-19 | 0.2 |
| glioma U251 | 0.1 |
| glioma SF-295 | 0.0 |
| Heart | 0.1 |
| Skeletal muscle | 0.2 |
| Bone marrow | 0.0 |
| Thymus | 0.7 |
| Spleen | 0.0 |
| Lymph node | 0.1 |
| Colon (ascending) | 1.3 |
| Stomach | 0.0 |
| Small intestine | 0.0 |
| Colon ca. SW480 | 0.1 |
| Colon ca.* SW620 (SW480 met) | 0.0 |
| Colon ca. HT29 | 0.0 |
| Colon ca. HCT-116 | 0.0 |
| Colon ca. CaCo-2 | 0.0 |
| Colon ca. HCT-15 | 0.2 |
| Colon ca. HCC-2998 | 0.0 |
| Gastric ca. * (liver met) NCI-N87 | 0.0 |
| Bladder | 0.1 |
| Trachea | 100.0 |
| Kidney | 0.0 |
| Kidney (fetal) | 0.0 |
| Renal ca. 786-0 | 0.0 |
| Renal ca. A498 | 0.1 |
| Renal ca. RXF 393 | 0.0 |
| Renal ca. ACHN | 0.0 |
| Renal ca. UO-31 | 0.2 |
| Renal ca. TK-10 | 0.1 |
| Liver | 0.0 |
| Liver (fetal) | 0.0 |
| Liver ca. (hepatoblast) HepG2 | 0.0 |
| Lung | 0.0 |
| Lung (fetal) | 0.0 |
| Lung ca. (small cell) LX-1 | 0.0 |
| Lung ca. (small cell) NCI-H69 | 0.6 |
| Lung ca. (s. cell var.) SHP-77 | 0.0 |
| Lung ca. (large cell)NCI-H460 | 0.0 |
| Lung ca. (non-sm. cell) A549 | 0.1 |
| Lung ca. (non-s. cell) NCI-H23 | 0.1 |
| Lung ca. (non-s. cell) HOP-62 | 0.0 |
| Lung ca. (non-s. cl) NCI-H522 | 0.0 |
| Lung ca. (squam.) SW 900 | 0.2 |
| Lung ca. (squam.) NCI-H596 | 0.3 |
| Mammary gland | 0.0 |
| Breast ca.* (pl. ef) MCF-7 | 0.0 |
| Breast ca.* (pl. ef) MDA-MB-231 | 0.2 |
| Breast ca.* (pl. ef) T47D | 0.9 |
| Breast ca. BT-549 | 0.0 |
| Breast ca. MDA-N | 0.0 |
| Ovary | 0.0 |
| Ovarian ca. OVCAR-3 | 0.1 |
| Ovarian ca. OVCAR-4 | 0.0 |
| Ovarian ca. OVCAR-5 | 0.6 |
| Ovarian ca. OVCAR-8 | 0.0 |
| Ovarian ca. IGROV-1 | 0.0 |
| Ovarian ca. (ascites) SK-OV-3 | 0.0 |
| Uterus | 0.0 |
| Placenta | 0.0 |
| Prostate | 0.0 |
| Prostate ca.* (bone met) PC-3 | 0.0 |
| Testis | 3.6 |
| Melanoma Hs688(A).T | 0.0 |
| Melanoma* (met) Hs688(B).T | 0.0 |
| Melanoma UACC-62 | 0.0 |
| Melanoma M14 | 0.2 |
| Melanoma LOX IMVI | 0.1 |
| Melanoma* (met) SK-MEL-5 | 0.0 |
| Melanoma SK-MEL-28 | 0.0 |

Column A - Rel. Exp. (%) Ag5, Run 87354971

TABLE FD
Panel 1.3D

| Tissue Name | A |
|---|---|
| Liver adenocarcinoma | 0.0 |
| Pancreas | 0.0 |
| Pancreatic ca. CAPAN 2 | 0.0 |
| Adrenal gland | 0.0 |
| Thyroid | 2.1 |
| Salivary gland | 12.9 |
| Pituitary gland | 0.0 |
| Brain (fetal) | 0.0 |
| Brain (whole) | 0.8 |
| Brain (amygdala) | 0.5 |
| Brain (cerebellum) | 0.0 |
| Brain (hippocampus) | 0.5 |
| Brain (substantia nigra) | 0.0 |
| Brain (thalamus) | 0.0 |
| Cerebral Cortex | 0.0 |
| Spinal cord | 100.0 |
| glio/astro U87-MG | 0.0 |
| glio/astro U-118-MG | 0.0 |
| astrocytoma SW1783 | 0.0 |
| neuro*; met SK-N-AS | 0.0 |
| astrocytoma SF-539 | 0.0 |
| astrocytoma SNB-75 | 0.0 |
| glioma SNB-19 | 0.4 |
| glioma U251 | 0.4 |
| glioma SF-295 | 0.0 |
| Heart (fetal) | 0.0 |
| Heart | 0.0 |
| Skeletal muscle (fetal) | 0.0 |
| Skeletal muscle | 0.0 |
| Bone marrow | 0.0 |
| Thymus | 8.9 |
| Spleen | 0.6 |
| Lymph node | 2.7 |
| Colorectal | 0.2 |
| Stomach | 44.8 |
| Small intestine | 0.0 |
| Colon ca. SW480 | 0.3 |
| Colon ca.* SW620(SW480 met) | 0.0 |
| Colon ca. HT29 | 0.0 |
| Colon ca. HCT-116 | 0.0 |
| Colon ca. CaCo-2 | 0.0 |
| Colon ca. tissue (ODO3866) | 0.0 |
| Colon ca. HCC-2998 | 0.0 |
| Gastric ca.* (liver met) NCI-N87 | 0.0 |
| Bladder | 0.0 |
| Trachea | 19.5 |
| Kidney | 0.0 |
| Kidney (fetal) | 0.3 |
| Renal ca. 786-0 | 0.0 |
| Renal ca. A498 | 0.2 |
| Renal ca. RXF 393 | 0.0 |
| Renal ca. ACHN | 0.0 |
| Renal ca. UO-31 | 0.0 |
| Renal ca. TK-10 | 0.0 |
| Liver | 0.0 |
| Liver (fetal) | 0.0 |
| Liver ca. (hepatoblast) HepG2 | 0.0 |
| Lung | 0.3 |
| Lung (fetal) | 0.0 |
| Lung ca. (small cell) LX-1 | 0.0 |
| Lung ca. (small cell) NCI-H69 | 0.0 |
| Lung ca. (s. cell var.) SHP-77 | 0.0 |
| Lung ca. (large cell)NCI-H460 | 0.4 |
| Lung ca. (non-sm. cell) A549 | 0.0 |
| Lung ca. (non-s. cell) NCI-H23 | 0.0 |
| Lung ca. (non-s. cell) HOP-62 | 0.0 |
| Lung ca. (non-s. cl) NCI-H522 | 0.0 |
| Lung ca. (squam.) SW 900 | 0.0 |
| Lung ca. (squam.) NCI-H596 | 0.0 |
| Mammary gland | 46.7 |
| Breast ca.* (pl. ef) MCF-7 | 0.0 |
| Breast ca.* (pl. ef) MDA-MB-231 | 0.0 |
| Breast ca.* (pl. ef) T47D | 0.0 |
| Breast ca. BT-549 | 0.0 |
| Breast ca. MDA-N | 0.0 |
| Ovary | 0.0 |
| Ovarian ca. OVCAR-3 | 0.0 |
| Ovarian ca. OVCAR-4 | 0.0 |
| Ovarian ca. OVCAR-5 | 0.0 |
| Ovarian ca. OVCAR-8 | 0.0 |
| Ovarian ca. IGROV-1 | 0.0 |
| Ovarian ca.* (ascites) SK-OV-3 | 0.6 |
| Uterus | 2.1 |
| Placenta | 0.0 |
| Prostate | 2.4 |
| Prostate ca.* (bone met)PC-3 | 0.0 |
| Testis | 1.4 |
| Melanoma Hs688(A).T | 0.0 |
| Melanoma* (met) Hs688(B).T | 0.0 |
| Melanoma UACC-62 | 0.0 |
| Melanoma M14 | 0.0 |
| Melanoma LOX IMVI | 0.5 |
| Melanoma* (met) SK-MEL-5 | 0.0 |
| Adipose | 0.0 |

Column A - Rel. Exp. (%) Ag2752, Run 165527213

TABLE FE
Panel 2D

| Tissue Name | A |
|---|---|
| Normal Colon | 3.6 |
| CC Well to Mod Diff (ODO3866) | 0.0 |
| CC Margin (ODO3866) | 3.0 |
| CC Gr.2 rectosigmoid (ODO3868) | 0.9 |
| CC Margin (ODO3868) | 0.0 |
| CC Mod Diff (ODO3920) | 0.0 |
| CC Margin (ODO3920) | 0.0 |
| CC Gr.2 ascend colon (ODO3921) | 0.0 |
| CC Margin (ODO3921) | 0.0 |
| CC from Partial Hepatectomy (ODO4309) Mets | 0.0 |
| Liver Margin (ODO4309) | 0.0 |
| Colon mets to lung (OD04451-01) | 0.0 |
| Lung Margin (OD04451-02) | 0.0 |
| Normal Prostate 6546-1 | 100.0 |
| Prostate Cancer (OD04410) | 0.0 |
| Prostate Margin (OD04410) | 3.5 |
| Prostate Cancer (OD04720-01) | 0.0 |
| Prostate Margin (OD04720-02) | 0.0 |
| Normal Lung 061010 | 0.0 |
| Lung Met to Muscle (ODO4286) | 1.0 |
| Muscle Margin (ODO4286) | 0.0 |
| Lung Malignant Cancer (OD03126) | 0.0 |
| Lung Margin (OD03126) | 0.0 |
| Lung Cancer (OD04404) | 24.5 |
| Lung Margin (OD04404) | 0.0 |
| Lung Cancer (OD04565) | 0.0 |
| Lung Margin (OD04565) | 0.0 |
| Lung Cancer (OD04237-01) | 0.0 |
| Lung Margin (OD04237-02) | 0.0 |
| Ocular Mel Met to Liver (ODO4310) | 0.0 |
| Liver Margin (ODO4310) | 0.0 |
| Melanoma Mets to Lung (OD04321) | 2.7 |
| Lung Margin (OD04321) | 0.0 |
| Normal Kidney | 3.5 |
| Kidney Ca, Nuclear grade 2 (OD04338) | 1.2 |
| Kidney Margin (OD04338) | 2.1 |
| Kidney Ca Nuclear grade 1/2 (OD04339) | 0.0 |
| Kidney Margin (OD04339) | 0.8 |
| Kidney Ca, Clear cell type (OD04340) | 0.0 |
| Kidney Margin (OD04340) | 1.2 |
| Kidney Ca, Nuclear grade 3 (OD04348) | 0.0 |
| Kidney Margin (OD04348) | 2.0 |
| Kidney Cancer (OD04622-01) | 0.0 |
| Kidney Margin (OD04622-03) | 0.0 |
| Kidney Cancer (OD04450-01) | 0.9 |
| Kidney Margin (OD04450-03) | 0.0 |
| Kidney Cancer 8120607 | 0.0 |
| Kidney Margin 8120608 | 0.0 |
| Kidney Cancer 8120613 | 0.0 |
| Kidney Margin 8120614 | 0.0 |
| Kidney Cancer 9010320 | 1.4 |
| Kidney Margin 9010321 | 3.9 |
| Normal Uterus | 0.0 |
| Uterus Cancer 064011 | 0.0 |
| Normal Thyroid | 62.9 |
| Thyroid Cancer 064010 | 0.0 |
| Thyroid Cancer A302152 | 2.1 |
| Thyroid Margin A302153 | 6.6 |
| Normal Breast | 0.0 |
| Breast Cancer (OD04566) | 0.0 |
| Breast Cancer (OD04590-01) | 0.0 |
| Breast Cancer Mets (OD04590-03) | 0.0 |
| Breast Cancer Metastasis (OD04655-05) | 0.0 |
| Breast Cancer 064006 | 0.0 |
| Breast Cancer 1024 | 0.0 |
| Breast Cancer 9100266 | 0.0 |
| Breast Margin 9100265 | 0.0 |
| Breast Cancer A209073 | 0.0 |
| Breast Margin A209073 | 0.0 |
| Normal Liver | 0.0 |
| Liver Cancer 064003 | 5.0 |
| Liver Cancer 1025 | 0.0 |
| Liver Cancer 1026 | 0.0 |
| Liver Cancer 6004-T | 0.0 |
| Liver Tissue 6004-N | 0.0 |
| Liver Cancer 6005-T | 0.0 |
| Liver Tissue 6005-N | 0.0 |
| Normal Bladder | 3.3 |
| Bladder Cancer 1023 | 0.0 |
| Bladder Cancer A302173 | 3.3 |
| Bladder Cancer (OD04718-01) | 0.0 |
| Bladder Normal Adjacent (OD04718-03) | 2.6 |
| Normal Ovary | 0.0 |
| Ovarian Cancer 064008 | 0.0 |
| Ovarian Cancer (OD04768-07) | 0.0 |
| Ovary Margin (OD04768-08) | 0.0 |
| Normal Stomach | 0.8 |
| Gastric Cancer 9060358 | 0.0 |
| Stomach Margin 9060359 | 0.0 |
| Gastric Cancer 9060395 | 0.0 |
| Stomach Margin 9060394 | 0.0 |
| Gastric Cancer 9060397 | 0.0 |
| Stomach Margin 9060396 | 0.0 |
| Gastric Cancer 064005 | 0.0 |

Column A - Rel. Exp. (%) Ag2752, Run 162555825

TABLE FF
Panel 4D

| Tissue Name | A |
|---|---|
| Secondary Th1 act | 0.0 |
| Secondary Th2 act | 0.0 |
| Secondary Tr1 act | 0.0 |
| Secondary Th1 rest | 0.0 |
| Secondary Th2 rest | 0.0 |
| Secondary Tr1 rest | 0.0 |
| Primary Th1 act | 0.0 |
| Primary Th2 act | 0.0 |
| Primary Tr1 act | 0.0 |
| Primary Th1 rest | 0.0 |
| Primary Th2 rest | 0.0 |
| Primary Tr1 rest | 0.0 |
| CD45RA CD4 lymphocyte act | 0.0 |
| CD45RO CD4 lymphocyte act | 0.0 |
| CD8 lymphocyte act | 0.0 |
| Secondary CD8 lymphocyte rest | 0.0 |
| Secondary CD8 lymphocyte act | 0.0 |
| CD4 lymphocyte none | 0.0 |
| 2ry Th1/Th2/Tr1_anti-CD95 CH11 | 0.0 |
| LAK cells rest | 0.0 |
| LAK cells IL-2 | 0.0 |
| LAK cells IL-2 + IL-12 | 0.0 |
| LAK cells IL-2 + IFN gamma | 0.0 |
| LAK cells IL-2 + IL-18 | 0.0 |
| LAK cells PMA/ionomycin | 0.0 |
| NK Cells IL-2 rest | 0.0 |
| Two Way MLR 3 day | 0.0 |
| Two Way MLR 5 day | 0.0 |
| Two Way MLR 7 day | 0.0 |
| PBMC rest | 0.0 |
| PBMC PWM | 0.0 |
| PBMC PHA-L | 0.0 |
| Ramos (B cell) none | 0.0 |
| Ramos (B cell) ionomycin | 0.0 |
| B lymphocytes PWM | 0.0 |
| B lymphocytes CD40L and IL-4 | 2.6 |
| EOL-1 dbcAMP | 0.0 |
| EOL-1 dbcAMP PMA/ionomycin | 0.0 |
| Dendritic cells none | 0.0 |
| Dendritic cells LPS | 0.0 |
| Dendritic cells anti-CD40 | 0.0 |
| Monocytes rest | 0.0 |
| Monocytes LPS | 0.0 |
| Macrophages rest | 0.0 |
| Macrophages LPS | 0.0 |
| HUVEC none | 0.0 |
| HUVEC starved | 0.0 |
| HUVEC IL-1beta | 0.0 |
| HUVEC IFN gamma | 0.0 |
| HUVEC TNF alpha + IFN gamma | 0.0 |
| HUVEC TNF alpha + IL4 | 0.0 |
| HUVEC IL-11 | 0.0 |
| Lung Microvascular EC none | 1.9 |
| Lung Microvascular EC TNFalpha + IL-1beta | 0.0 |
| Microvascular Dermal EC none | 0.0 |
| Microsvasular Dermal EC TNFalpha + IL-1beta | 0.0 |
| Bronchial epithelium TNFalpha + IL1beta | 0.0 |
| Small airway epithelium none | 100.0 |
| Small airway epithelium TNFalpha + IL-1beta | 80.7 |
| Coronery artery SMC rest | 1.7 |
| Coronery artery SMC TNFalpha + IL-1beta | 0.0 |
| Astrocytes rest | 0.0 |
| Astrocytes TNFalpha + IL-1beta | 0.0 |
| KU-812 (Basophil) rest | 0.0 |
| KU-812 (Basophil) PMA/ionomycin | 0.0 |
| CCD1106 (Keratinocytes) none | 0.0 |
| CCD1106 (Keratinocytes) TNFalpha + IL-1beta | 0.0 |
| Liver cirrhosis | 8.1 |
| Lupus kidney | 0.0 |
| NCI-H292 none | 0.0 |
| NCI-H292 IL-4 | 0.0 |
| NCI-H292 IL-9 | 0.0 |
| NCI-H292 IL-13 | 3.2 |
| NCI-H292 IFN gamma | 0.0 |
| HPAEC none | 0.0 |
| HPAEC TNF alpha + IL-1 beta | 0.0 |
| Lung fibroblast none | 0.0 |
| Lung fibroblast TNF alpha + IL-1 beta | 0.0 |
| Lung fibroblast IL-4 | 0.0 |
| Lung fibroblast IL-9 | 0.0 |
| Lung fibroblast IL-13 | 0.0 |
| Lung fibroblast IFN gamma | 0.0 |
| Dermal fibroblast CCD1070 rest | 0.0 |
| Dermal fibroblast CCD1070 TNF alpha | 0.3 |
| Dermal fibroblast CCD1070 IL-1 beta | 0.0 |
| Dermal fibroblast IFN gamma | 0.0 |
| Dermal fibroblast IL-4 | 0.0 |
| IBD Colitis 2 | 0.0 |
| IBD Crohn's | 0.0 |
| Colon | 0.0 |
| Lung | 4.7 |
| Thymus | 0.0 |
| Kidney | 3.6 |

Column A - Rel. Exp. (%) Ag2752, Run 162015236

Panel 1 Summary: Ag5 Highest CG50513-01 gene expression was seen in trachea (CT=25.2) with low to moderate expression detected in normal tissues including: testis, colon, thymus, skeletal muscle, spinal cord, pituitary gland, salivary gland, thyroid, and adrenal gland. Therefore, therapeutic modulation of this gene, encoded protein and/or antibodies, small molecule drug targeting the protein are useful in the treatment of the diseases associated with these tissues including obesity, diabetes, fertility and hypogonadism. Low gene expression was also seen in breast, lung and brain cancer cell lines. Gene expression level is a marker of these cancer tissues and for detecting the presence of these cancers in vitro or in vivo. Therapeutic modulation of this gene gene, encoded protein and/or antibodies, small molecule drug targeting the protein is useful in the treatment of these cancers.

Panel 2D Summary: Ag2752 Highest gene expression was seen in normal prostate (CT=31.4) and significant expression was also seen in normal prostate and thyroid compared to cancer samples. Therapeutic modulation of this gene and/or encoded protein that increases the activity of this gene and the encoded protein are useful in the treatment of prostate and thyroid cancers.

Low gene expression was also detected in lung cancer which was higher than expression in normal lung. Therefore, expression level of this gene will be useful as marker to detect the presence of lung cancer and therapeutic modulation of this gene, encoded protein is useful in the treatment of lung cancer.

Panel 4D Summary: Ag2752 Significant gene expression was detected in resting and activated small airway epithelium (CTs=31.5) and modulation of the expression or activity of this gene and/or the protein encoded by it is useful in the treatment of asthma, COPD, and emphysema.

G. CG50949-03: Membrane-Type Mosaic Serine Protease.

Expression of gene CG50949-03 was assessed using the primer-probe sets Ag020b, Ag20 and Ag5238, described in Tables GA, GB and GC. Results of the RTQ-PCR runs are shown in Tables GD, GE and GF.

TABLE GA

Probe Name Ag020b

| | | | | |
|---|---|---|---|---|
| | | | | SEQ ID |
| Primers | Sequences | Length | Start Position | No |

| | | | | |
|---|---|---|---|---|
| Forward | 5′-gtgggaacactggagggagat-3′ | 21 | 1039 | 370 |
| Probe | TET-5′-aggtctgaatgcccttcccagcg-3′- | 23 | 1012 | 371 |
| | TAMRA | | | |
| Reverse | 5′-caactccaccatccaggaaag-3′ | 21 | 984 | 372 |

TABLE GB

Probe Name Ag20

| | | | | |
|---|---|---|---|---|
| | | | | SEQ ID |
| Primers | Sequences | Length | Start Position | No |

| | | | | |
|---|---|---|---|---|
| Forward | 5′-gtgggaacactggagggagat-3′ | 21 | 1039 | 373 |
| Probe | TET-5′-aggtctgaatgcccttcccagcg-3′ | 23 | 1012 | 374 |
| | TAMRA | | | |
| Reverse | 5′-caactccaccatccaggaaag-3′ | 21 | 984 | 375 |

TABLE GC

Probe Name Ag5238

| | | | | |
|---|---|---|---|---|
| Primers | Sequences | Length | Start Position | SEQ ID No |

| | | | | |
|---|---|---|---|---|
| Forward | 5′-ggaggtaagatccctgcagc-3′ | 20 | 1785 | 376 |
| Probe | TET-5′-acttcctcaggtggggaccct-3′-TAMRA | 21 | 1831 | 377 |
| Reverse | 5′-tgaggggacctctgcctaca-3′ | 20 | 1868 | 378 |

TABLE GD
General_screening_panel_v1.5

| Tissue Name | A |
|---|---|
| Adipose | 1.9 |
| Melanoma* Hs688(A).T | 0.0 |
| Melanoma* Hs688(B).T | 0.0 |
| Melanoma* M14 | 61.6 |
| Melanoma* LOXIMVI | 0.0 |
| Melanoma* SK-MEL-5 | 0.0 |
| Squamous cell carcinoma SCC-4 | 0.0 |
| Testis Pool | 1.3 |
| Prostate ca.* (bone met) PC-3 | 0.0 |
| Prostate Pool | 7.3 |
| Placenta | 28.7 |
| Uterus Pool | 0.0 |
| Ovarian ca. OVCAR-3 | 5.8 |
| Ovarian ca. SK-OV-3 | 0.0 |
| Ovarian ca. OVCAR-4 | 3.0 |
| Ovarian ca. OVCAR-5 | 10.3 |
| Ovarian ca. IGROV-1 | 0.0 |
| Ovarian ca. OVCAR-8 | 0.0 |
| Ovary | 2.8 |
| Breast ca. MCF-7 | 63.3 |
| Breast ca. MDA-MB-231 | 0.0 |
| Breast ca. BT 549 | 4.7 |
| Breast ca. T47D | 100.0 |
| Breast ca. MDA-N | 0.0 |
| Breast Pool | 0.0 |
| Trachea | 9.0 |
| Lung | 0.0 |
| Fetal Lung | 4.7 |
| Lung ca. NCI-N417 | 0.0 |
| Lung ca. LX-1 | 0.0 |
| Lung ca. NCI-H146 | 4.0 |
| Lung ca. SHP-77 | 0.0 |
| Lung ca. A549 | 0.0 |
| Lung ca. NCI-H526 | 1.9 |
| Lung ca. NCI-H23 | 3.3 |
| Lung ca. NCI-H460 | 87.1 |
| Lung ca. HOP-62 | 0.0 |
| Lung ca. NCI-H522 | 0.0 |
| Liver | 0.0 |
| Fetal Liver | 6.3 |
| Liver ca. HepG2 | 0.0 |
| Kidney Pool | 1.5 |
| Fetal Kidney | 2.5 |
| Renal ca. 786-0 | 0.0 |
| Renal ca. A498 | 0.0 |
| Renal ca. ACHN | 0.0 |
| Renal ca. UO-31 | 0.0 |
| Renal ca. TK-10 | 0.0 |
| Bladder | 16.0 |
| Gastric ca. (liver met.) NCI-N87 | 55.9 |
| Gastric ca. KATO III | 0.0 |
| Colon ca. SW-948 | 1.2 |
| Colon ca. SW480 | 0.0 |
| Colon ca.* (SW480 met) SW620 | 1.5 |
| Colon ca. HT29 | 2.0 |
| Colon ca. HCT-116 | 2.1 |
| Colon ca. CaCo-2 | 19.3 |
| Colon cancer tissue | 7.9 |
| Colon ca. SW1116 | 0.0 |
| Colon ca. Colo-205 | 3.7 |
| Colon ca. SW-48 | 0.0 |
| Colon Pool | 7.3 |
| Small Intestine Pool | 3.6 |
| Stomach Pool | 4.7 |
| Bone Marrow Pool | 1.8 |
| Fetal Heart | 6.3 |
| Heart Pool | 1.6 |
| Lymph Node Pool | 4.2 |
| Fetal Skeletal Muscle | 0.0 |
| Skeletal Muscle Pool | 0.0 |
| Spleen Pool | 0.0 |
| Thymus Pool | 0.0 |
| CNS cancer (glio/astro) U87-MG | 0.0 |
| CNS cancer (glio/astro) U-118-MG | 0.0 |
| CNS cancer (neuro; met) SK-N-AS | 0.0 |
| CNS cancer (astro) SF-539 | 0.0 |
| CNS cancer (astro) SNB-75 | 0.0 |
| CNS cancer (glio) SNB-19 | 0.0 |
| CNS cancer (glio) SF-295 | 0.0 |
| Brain (Amygdala) Pool | 0.0 |
| Brain (cerebellum) | 0.0 |
| Brain (fetal) | 2.9 |
| Brain (Hippocampus) Pool | 2.3 |
| Cerebral Cortex Pool | 0.0 |
| Brain (Substantia nigra) Pool | 2.6 |
| Brain (Thalamus) Pool | 0.0 |
| Brain (whole) | 0.0 |
| Spinal Cord Pool | 0.0 |
| Adrenal Gland | 2.4 |
| Pituitary gland Pool | 0.0 |
| Salivary Gland | 6.1 |
| Thyroid (female) | 0.0 |
| Pancreatic ca. CAPAN2 | 0.0 |
| Pancreas Pool | 7.0 |

Column A - Rel. Exp. (%) Ag5238, Run 229665050

TABLE GE
General_screening_panel_v1.6

| Tissue Name | A |
|---|---|
| Adipose | 0.2 |
| Melanoma* Hs688(A).T | 0.0 |
| Melanoma* Hs688(B).T | 0.0 |
| Melanoma* M14 | 9.4 |
| Melanoma* LOXIMVI | 0.4 |
| Melanoma* SK-MEL-5 | 0.8 |
| Squamous cell carcinoma SCC-4 | 1.8 |
| Testis Pool | 0.3 |
| Prostate ca.* (bone met) PC-3 | 0.3 |
| Prostate Pool | 0.9 |
| Placenta | 30.1 |
| Uterus Pool | 0.8 |
| Ovarian ca. OVCAR-3 | 9.5 |
| Ovarian ca. SK-OV-3 | 0.1 |
| Ovarian ca. OVCAR-4 | 1.6 |
| Ovarian ca. OVCAR-5 | 35.6 |
| Ovarian ca. IGROV-1 | 0.2 |
| Ovarian ca. OVCAR-8 | 0.0 |
| Ovary | 0.2 |
| Breast ca. MCF-7 | 77.9 |
| Breast ca. MDA-MB-231 | 0.2 |
| Breast ca. BT 549 | 0.4 |
| Breast ca. T47D | 100.0 |
| Breast ca. MDA-N | 0.3 |
| Breast Pool | 0.5 |
| Trachea | 6.9 |
| Lung | 0.1 |
| Fetal Lung | 9.0 |
| Lung ca. NCI-N417 | 0.0 |
| Lung ca. LX-1 | 2.3 |
| Lung ca. NCI-H146 | 0.4 |
| Lung ca. SHP-77 | 0.2 |
| Lung ca. A549 | 0.5 |
| Lung ca. NCI-H526 | 0.4 |
| Lung ca. NCI-H23 | 0.8 |
| Lung ca. NCI-H460 | 0.4 |
| Lung ca. HOP-62 | 0.2 |
| Lung ca. NCI-H522 | 0.3 |
| Liver | 0.2 |
| Fetal Liver | 2.3 |
| Liver ca. HepG2 | 0.0 |
| Kidney Pool | 0.3 |
| Fetal Kidney | 0.8 |
| Renal ca. 786-0 | 0.2 |
| Renal ca. A498 | 0.2 |
| Renal ca. ACHN | 1.1 |
| Renal ca. UO-31 | 0.8 |
| Renal ca. TK-10 | 0.1 |
| Bladder | 3.9 |
| Gastric ca. (liver met.) NCI-N87 | 42.3 |
| Gastric ca. KATO III | 1.5 |
| Colon ca. SW-948 | 0.5 |
| Colon ca. SW480 | 0.6 |
| Colon ca.* (SW480 met) SW620 | 0.3 |
| Colon ca. HT29 | 6.8 |
| Colon ca. HCT-116 | 2.0 |
| Colon ca. CaCo-2 | 92.7 |
| Colon cancer tissue | 23.3 |
| Colon ca. SW1116 | 0.2 |
| Colon ca. Colo-205 | 8.2 |
| Colon ca. SW-48 | 1.8 |
| Colon Pool | 0.7 |
| Small Intestine Pool | 1.7 |
| Stomach Pool | 1.0 |
| Bone Marrow Pool | 1.3 |
| Fetal Heart | 0.1 |
| Heart Pool | 0.0 |
| Lymph Node Pool | 0.7 |
| Fetal Skeletal Muscle | 0.1 |
| Skeletal Muscle Pool | 0.2 |
| Spleen Pool | 1.0 |
| Thymus Pool | 1.2 |
| CNS cancer (glio/astro) U87-MG | 0.1 |
| CNS cancer (glio/astro) U-118-MG | 0.3 |
| CNS cancer (neuro; met) SK-N-AS | 0.0 |
| CNS cancer (astro) SF-539 | 0.6 |
| CNS cancer (astro) SNB-75 | 0.0 |
| CNS cancer (glio) SNB-19 | 0.4 |
| CNS cancer (glio) SF-295 | 0.7 |
| Brain (Amygdala) Pool | 0.2 |
| Brain (cerebellum) | 0.3 |
| Brain (fetal) | 1.5 |
| Brain (Hippocampus) Pool | 1.4 |
| Cerebral Cortex Pool | 0.2 |
| Brain (Substantia nigra) Pool | 0.2 |
| Brain (Thalamus) Pool | 0.4 |
| Brain (whole) | 0.1 |
| Spinal Cord Pool | 0.2 |
| Adrenal Gland | 0.2 |
| Pituitary gland Pool | 0.2 |
| Salivary Gland | 8.8 |
| Thyroid (female) | 6.0 |
| Pancreatic ca. CAPAN2 | 1.9 |
| Pancreas Pool | 3.0 |

Column A - Rel. Exp. (%) Ag20, Run 277226634

TABLE GF
Panel 4.1D

| Tissue Name | A | B |
|---|---|---|
| Secondary Th1 act | 2.3 | 1.9 |
| Secondary Th2 act | 12.0 | 1.8 |
| Secondary Tr1 act | 7.7 | 10.9 |
| Secondary Th1 rest | 2.4 | 2.0 |
| Secondary Th2 rest | 4.2 | 0.0 |
| Secondary Tr1 rest | 3.7 | 0.0 |
| Primary Th1 act | 4.5 | 4.7 |
| Primary Th2 act | 7.4 | 2.4 |
| Primary Tr1 act | 6.1 | 0.0 |
| Primary Th1 rest | 1.4 | 0.0 |
| Primary Th2 rest | 2.8 | 0.0 |
| Primary Tr1 rest | 1.7 | 0.0 |
| CD45RA CD4 lymphocyte act | 6.1 | 0.0 |
| CD45RO CD4 lymphocyte act | 6.7 | 3.4 |
| CD8 lymphocyte act | 8.4 | 0.0 |
| Secondary CD8 lymphocyte rest | 7.0 | 2.5 |
| Secondary CD8 lymphocyte act | 1.2 | 0.0 |
| CD4 lymphocyte none | 6.8 | 0.0 |
| 2ry Th1/Th2/Tr1_anti-CD95 CH11 | 6.6 | 0.0 |
| LAK cells rest | 2.6 | 8.7 |
| LAK cells IL-2 | 3.2 | 0.0 |
| LAK cells IL-2 + IL-12 | 2.2 | 11.0 |
| LAK cells IL-2 + IFN gamma | 2.5 | 0.0 |
| LAK cells IL-2 + IL-18 | 0.9 | 2.0 |
| LAK cells PMA/ionomycin | 2.3 | 1.8 |
| NK Cells IL-2 rest | 12.5 | 1.6 |
| Two Way MLR 3 day | 6.3 | 4.5 |
| Two Way MLR 5 day | 1.8 | 0.0 |
| Two Way MLR 7 day | 4.1 | 16.7 |
| PBMC rest | 5.9 | 2.0 |
| PBMC PWM | 0.0 | 9.7 |
| PBMC PHA-L | 5.8 | 0.0 |
| Ramos (B cell) none | 1.5 | 0.0 |
| Ramos (B cell) ionomycin | 7.8 | 0.0 |
| B lymphocytes PWM | 1.3 | 1.7 |
| B lymphocytes CD40L and IL-4 | 3.6 | 0.0 |
| EOL-1 dbcAMP | 5.4 | 0.0 |
| EOL-1 dbcAMP PMA/ionomycin | 0.0 | 2.5 |
| Dendritic cells none | 6.2 | 0.0 |
| Dendritic cells LPS | 10.7 | 3.0 |
| Dendritic cells anti-CD40 | 3.0 | 0.0 |
| Monocytes rest | 1.2 | 40.6 |
| Monocytes LPS | 27.9 | 0.0 |
| Macrophages rest | 3.2 | 0.0 |
| Macrophages LPS | 10.4 | 100.0 |
| HUVEC none | 0.4 | 3.1 |
| HUVEC starved | 2.8 | 3.5 |
| HUVEC IL-1beta | 1.1 | 0.0 |
| HUVEC IFN gamma | 1.2 | 0.0 |
| HUVEC TNF alpha + IFN gamma | 2.1 | 23.3 |
| HUVEC TNF alpha + IL4 | 1.4 | 0.0 |
| HUVEC IL-11 | 2.8 | 0.0 |
| Lung Microvascular EC none | 3.7 | 0.0 |
| Lung Microvascular EC TNFalpha + | 0.0 | 2.1 |
| IL-1beta | | |
| Microvascular Dermal EC none | 0.6 | 0.0 |
| Microsvasular Dermal EC TNFalpha + | 4.4 | 2.1 |
| IL-1beta | | |
| Bronchial epithelium TNFalpha + | 0.0 | 0.0 |
| IL1beta | | |
| Small airway epithelium none | 34.2 | 0.0 |
| Small airway epithelium TNFalpha + | 25.9 | 0.0 |
| IL-1beta | | |
| Coronery artery SMC rest | 4.8 | 0.0 |
| Coronery artery SMC TNFalpha + IL- | 0.0 | 0.0 |
| 1beta | | |
| Astrocytes rest | 0.7 | 0.0 |
| Astrocytes TNFalpha + IL-1beta | 1.5 | 1.5 |
| KU-812 (Basophil) rest | 5.0 | 1.5 |
| KU-812 (Basophil) PMA/ionomycin | 0.8 | 0.0 |
| CCD1106 (Keratinocytes) none | 5.1 | 27.9 |
| CCD1106 (Keratinocytes) TNFalpha + | 10.0 | 37.9 |
| IL-1beta | | |
| Liver cirrhosis | 6.7 | 3.4 |
| NCI-H292 none | 100.0 | 0.0 |
| NCI-H292 IL-4 | 40.1 | 0.0 |
| NCI-H292 IL-9 | 98.6 | 19.9 |
| NCI-H292 IL-13 | 73.7 | 0.0 |
| NCI-H292 IFN gamma | 49.0 | 0.0 |
| HPAEC none | 8.5 | 0.0 |
| HPAEC TNF alpha + IL-1 beta | 4.7 | 0.0 |
| Lung fibroblast none | 2.8 | 0.0 |
| Lung fibroblast TNF alpha + IL-1 beta | 3.7 | 4.7 |
| Lung fibroblast IL-4 | 2.5 | 0.0 |
| Lung fibroblast IL-9 | 4.1 | 1.9 |
| Lung fibroblast IL-13 | 0.0 | 0.0 |
| Lung fibroblast IFN gamma | 1.4 | 0.0 |
| Dermal fibroblast CCD1070 rest | 1.3 | 2.7 |
| Dermal fibroblast CCD1070 TNF alpha | 9.1 | 0.0 |
| Dermal fibroblast CCD1070 IL-1 beta | 2.8 | 0.0 |
| Dermal fibroblast IFN gamma | 3.1 | 0.0 |
| Dermal fibroblast IL-4 | 2.7 | 0.0 |
| Dermal Fibroblasts rest | 0.3 | 0.0 |
| Neutrophils TNFa + LPS | 1.7 | 0.0 |
| Neutrophils rest | 6.6 | 0.0 |
| Colon | 3.1 | 0.0 |
| Lung | 3.1 | 33.9 |
| Thymus | 0.0 | 0.0 |
| Kidney | 4.5 | 5.9 |

Column A - Rel. Exp. (%) Ag20, Run 268789078
Column B - Rel. Exp. (%) Ag5238, Run 229819576

General_screening_panel_V.5 Summary: Ag5238 Highest gene expression was detected in T47D breast cancer cell line (CT=32.7) and low expression was also seen in melanoma, lung, gastric, and breast cancers cell lines. Expression level is a useful marker to differentiate these cancers from normal tissues and to detect the presence of these cancers in vitro or in vivo.

General_screening_panel_v1.6 Summary: Ag20 Highest gene expression was detected in T47D breast cancer cell line (CT=26). Moderate to low expression was also seen in melanoma, lung, colon, renal, pancreatic, renal, brain, gastric, and breast cancers cell lines. Expression is a useful marker to differentiate these cancers from normal tissues and to detect the presence of these cancers in vitro or in vivo.

Among tissues with metabolic or endocrine function, this gene was expressed at moderate to low levels in pancreas, adipose, thyroid, fetal liver and the gastrointestinal tract. Therefore, therapeutic modulation of the activity of this gene is useful in the treatment of endocrine/metabolically related diseases, such as obesity and diabetes. This gene was expressed at low levels in most regions of the central nervous system examined including: amygdala, hippocampus, thalamus, cerebellum, and spinal cord. Therefore, therapeutic modulation of this gene and/or expressed protein is useful in the treatment of central nervous system disorders such as Alzheimer's disease, Parkinson's disease, epilepsy, multiple sclerosis, schizophrenia and depression.

Panel 4.1D Summary: Ag20 Highest expression of this gene was seen in resting NCI-H292 cells (CT=30) with significant expression also detected in activated NCI-H292 cells, small airway epithelium, activated monocytes and macrophages, dendritic cells, and resting IL2 treated NK cells. Therefore, modulation of this gene and/or expressed protein with a functional therapeutic will alter the functions associated with these cell types and will relieve the symptoms of patients suffering from autoimmune and inflammatory diseases such as asthma, allergies, inflammatory bowel disease, lupus erythematosus, psoriasis, rheumatoid arthritis, and osteoarthritis.Ag5238 Highest expression using this probe-primer set was detected in activated macrophages (CT=33). Low gene expression was also seen in resting monocytes, keratinocytes and lung. Therefore, therapeutics modulation of this gene and/or expressed protein is important for the treatment of asthma, emphysema, inflammatory bowel disease, arthritis and psoriasis.

H. CG50949-05 and CG50949-06: Mosaic Serine Protease.

Expression of gene CG50949-05 and CG50949-06 was assessed using the primer-probe sets Ag020b, Ag20 and Ag5241, described in Tables HA, HB and HC. Results of the RTQ-PCR runs are shown in Tables HD, HE and HF.

Table HA. Probe Name Ag020b

TABLE HA

Probe Name Ag020b

| | | | | |
|---|---|---|---|---|
| | | | | SEQ ID |
| Primers | Sequences | Length | Start Position | No |

| | | | | |
|---|---|---|---|---|
| Forward | 5′-gtgggaacactggagggagat-3′ | 21 | 925 | 379 |
| Probe | TET-5′-aggtctgaatgcccttcccagcg-3′- | 23 | 898 | 380 |
| | TAMRA | | | |
| Reverse | 5′-caactccaccatccaggaaag-3′ | 21 | 870 | 381 |

TABLE HB

Probe Name Ag20

| | | | | |
|---|---|---|---|---|
| | | | | SEQ ID |
| Primers | Sequences | Length | Start Position | No |

| | | | | |
|---|---|---|---|---|
| Forward | 5′-gtgggaacactggagggagat-3′ | 21 | 925 | 382 |
| Probe | TET-5′-aggtctgaatgcccttcccagcg-3′- | 23 | 898 | 383 |
| Reverse | 5′-caactccaccatccaggaaag-3′ | 21 | 870 | 384 |

Table HC. Probe Name Ag5241

TABLE HC

Probe Name Ag5241

| | | | | |
|---|---|---|---|---|
| | | | | SEQ ID |
| Primers | Sequences | Length | Start Position | No |

| | | | | |
|---|---|---|---|---|
| Forward | 5′-gagagcccagtccagttctgg-3′ | 21 | 544 | 385 |
| Probe | TET-5′-atcaggtacaaggagcagagggaga-3′- | 25 | 580 | 386 |
| | TAMRA | | | |
| Reverse | 5′-caccccgtcacagcgaaca-3′ | 19 | 621 | 387 |

TABLE HD
General_screening_panel_v1.5

| Tissue Name | A |
|---|---|
| Adipose | 0.3 |
| Melanoma* Hs688(A).T | 0.0 |
| Melanoma* Hs688(B).T | 0.0 |
| Melanoma* M14 | 38.2 |
| Melanoma* LOXIMVI | 0.8 |
| Melanoma* SK-MEL-5 | 2.1 |
| Squamous cell carcinoma SCC-4 | 1.9 |
| Testis Pool | 0.4 |
| Prostate ca.* (bone met) PC-3 | 0.0 |
| Prostate Pool | 4.2 |
| Placenta | 19.8 |
| Uterus Pool | 0.8 |
| Ovarian ca. OVCAR-3 | 9.3 |
| Ovarian ca. SK-OV-3 | 0.2 |
| Ovarian ca. OVCAR-4 | 1.2 |
| Ovarian ca. OVCAR-5 | 41.5 |
| Ovarian ca. IGROV-1 | 0.4 |
| Ovarian ca. OVCAR-8 | 0.0 |
| Ovary | 1.1 |
| Breast ca. MCF-7 | 72.7 |
| Breast ca. MDA-MB-231 | 0.0 |
| Breast ca. BT 549 | 0.0 |
| Breast ca. T47D | 100.0 |
| Breast ca. MDA-N | 0.7 |
| Breast Pool | 1.8 |
| Trachea | 10.7 |
| Lung | 0.0 |
| Fetal Lung | 28.9 |
| Lung ca. NCI-N417 | 0.0 |
| Lung ca. LX-1 | 0.9 |
| Lung ca. NCI-H146 | 3.0 |
| Lung ca. SHP-77 | 0.0 |
| Lung ca. A549 | 0.8 |
| Lung ca. NCI-H526 | 0.0 |
| Lung ca. NCI-H23 | 1.7 |
| Lung ca. NCI-H460 | 7.3 |
| Lung ca. HOP-62 | 0.0 |
| Lung ca. NCI-H522 | 0.3 |
| Liver | 0.0 |
| Fetal Liver | 0.0 |
| Liver ca. HepG2 | 0.0 |
| Kidney Pool | 4.0 |
| Fetal Kidney | 2.0 |
| Renal ca. 786-0 | 0.0 |
| Renal ca. A498 | 0.0 |
| Renal ca. ACHN | 3.1 |
| Renal ca. UO-31 | 0.4 |
| Renal ca. TK-10 | 0.0 |
| Bladder | 11.2 |
| Gastric ca. (liver met.) NCI-N87 | 36.1 |
| Gastric ca. KATO III | 0.5 |
| Colon ca. SW-948 | 0.0 |
| Colon ca. SW480 | 1.2 |
| Colon ca.* (SW480 met) SW620 | 0.0 |
| Colon ca. HT29 | 6.3 |
| Colon ca. HCT-116 | 1.1 |
| Colon ca. CaCo-2 | 95.3 |
| Colon cancer tissue | 16.6 |
| Colon ca. SW1116 | 0.0 |
| Colon ca. Colo-205 | 9.2 |
| Colon ca. SW-48 | 0.6 |
| Colon Pool | 2.9 |
| Small Intestine Pool | 1.1 |
| Stomach Pool | 1.1 |
| Bone Marrow Pool | 0.6 |
| Fetal Heart | 0.7 |
| Heart Pool | 0.0 |
| Lymph Node Pool | 0.7 |
| Fetal Skeletal Muscle | 1.1 |
| Skeletal Muscle Pool | 0.0 |
| Spleen Pool | 5.9 |
| Thymus Pool | 2.9 |
| CNS cancer (glio/astro) U87-MG | 0.0 |
| CNS cancer (glio/astro) U-118-MG | 0.0 |
| CNS cancer (neuro; met) SK-N-AS | 0.0 |
| CNS cancer (astro) SF-539 | 0.0 |
| CNS cancer (astro) SNB-75 | 0.0 |
| CNS cancer (glio) SNB-19 | 2.4 |
| CNS cancer (glio) SF-295 | 0.9 |
| Brain (Amygdala) Pool | 0.0 |
| Brain (cerebellum) | 1.4 |
| Brain (fetal) | 16.3 |
| Brain (Hippocampus) Pool | 3.0 |
| Cerebral Cortex Pool | 0.4 |
| Brain (Substantia nigra) Pool | 1.1 |
| Brain (Thalamus) Pool | 3.3 |
| Brain (whole) | 4.0 |
| Spinal Cord Pool | 1.5 |
| Adrenal Gland | 0.9 |
| Pituitary gland Pool | 0.0 |
| Salivary Gland | 5.7 |
| Thyroid (female) | 8.8 |
| Pancreatic ca. CAPAN2 | 1.1 |
| Pancreas Pool | 6.5 |

Column A - Rel. Exp. (%) Ag5241, Run 229665051

TABLE HE
General_screening_panel_v1.6

| Tissue Name | A |
|---|---|
| Adipose | 0.2 |
| Melanoma* Hs688(A).T | 0.0 |
| Melanoma* Hs688(B).T | 0.0 |
| Melanoma* M14 | 9.4 |
| Melanoma* LOXIMVI | 0.4 |
| Melanoma* SK-MEL-5 | 0.8 |
| Squamous cell carcinoma SCC-4 | 1.8 |
| Testis Pool | 0.3 |
| Prostate ca.* (bone met) PC-3 | 0.3 |
| Prostate Pool | 0.9 |
| Placenta | 30.1 |
| Uterus Pool | 0.8 |
| Ovarian ca. OVCAR-3 | 9.5 |
| Ovarian ca. SK-OV-3 | 0.1 |
| Ovarian ca. OVCAR-4 | 1.6 |
| Ovarian ca. OVCAR-5 | 35.6 |
| Ovarian ca. IGROV-1 | 0.2 |
| Ovarian ca. OVCAR-8 | 0.0 |
| Ovary | 0.2 |
| Breast ca. MCF-7 | 77.9 |
| Breast ca. MDA-MB-231 | 0.2 |
| Breast ca. BT 549 | 0.4 |
| Breast ca. T47D | 100.0 |
| Breast ca. MDA-N | 0.3 |
| Breast Pool | 0.5 |
| Trachea | 6.9 |
| Lung | 0.1 |
| Fetal Lung | 9.0 |
| Lung ca. NCI-N417 | 0.0 |
| Lung ca. LX-1 | 2.3 |
| Lung ca. NCI-H146 | 0.4 |
| Lung ca. SHP-77 | 0.2 |
| Lung ca. A549 | 0.5 |
| Lung ca. NCI-H526 | 0.4 |
| Lung ca. NCI-H23 | 0.8 |
| Lung ca. NCI-H460 | 0.4 |
| Lung ca. HOP-62 | 0.2 |
| Lung ca. NCI-H522 | 0.3 |
| Liver | 0.2 |
| Fetal Liver | 2.3 |
| Liver ca. HepG2 | 0.0 |
| Kidney Pool | 0.3 |
| Fetal Kidney | 0.8 |
| Renal ca. 786-0 | 0.2 |
| Renal ca. A498 | 0.2 |
| Renal ca. ACHN | 1.1 |
| Renal ca. UO-31 | 0.8 |
| Renal ca. TK-10 | 0.1 |
| Bladder | 3.9 |
| Gastric ca. (liver met.) NCI-N87 | 42.3 |
| Gastric ca. KATO III | 1.5 |
| Colon ca. SW-948 | 0.5 |
| Colon ca. SW480 | 0.6 |
| Colon ca.* (SW480 met) SW620 | 0.3 |
| Colon ca. HT29 | 6.8 |
| Colon ca. HCT-116 | 2.0 |
| Colon ca. CaCo-2 | 92.7 |
| Colon cancer tissue | 23.3 |
| Colon ca. SW1116 | 0.2 |
| Colon ca. Colo-205 | 8.2 |
| Colon ca. SW-48 | 1.8 |
| Colon Pool | 0.7 |
| Small Intestine Pool | 1.7 |
| Stomach Pool | 1.0 |
| Bone Marrow Pool | 1.3 |
| Fetal Heart | 0.1 |
| Heart Pool | 0.0 |
| Lymph Node Pool | 0.7 |
| Fetal Skeletal Muscle | 0.1 |
| Skeletal Muscle Pool | 0.2 |
| Spleen Pool | 1.0 |
| Thymus Pool | 1.2 |
| CNS cancer (glio/astro) U87-MG | 0.1 |
| CNS cancer (glio/astro) U-118-MG | 0.3 |
| CNS cancer (neuro; met) SK-N-AS | 0.0 |
| CNS cancer (astro) SF-539 | 0.6 |
| CNS cancer (astro) SNB-75 | 0.0 |
| CNS cancer (glio) SNB-19 | 0.4 |
| CNS cancer (glio) SF-295 | 0.7 |
| Brain (Amygdala) Pool | 0.2 |
| Brain (cerebellum) | 0.3 |
| Brain (fetal) | 1.5 |
| Brain (Hippocampus) Pool | 1.4 |
| Cerebral Cortex Pool | 0.2 |
| Brain (Substantia nigra) Pool | 0.2 |
| Brain (Thalamus) Pool | 0.4 |
| Brain (whole) | 0.1 |
| Spinal Cord Pool | 0.2 |
| Adrenal Gland | 0.2 |
| Pituitary gland Pool | 0.2 |
| Salivary Gland | 8.8 |
| Thyroid (female) | 6.0 |
| Pancreatic ca. CAPAN2 | 1.9 |
| Pancreas Pool | 3.0 |

Column A - Rel. Exp. (%) Ag20, Run 277226634

TABLE HF
Panel 4.1D

| Tissue Name | A | B |
|---|---|---|
| Secondary Th1 act | 2.3 | 6.1 |
| Secondary Th2 act | 12.0 | 22.8 |
| Secondary Tr1 act | 7.7 | 6.0 |
| Secondary Th1 rest | 2.4 | 0.0 |
| Secondary Th2 rest | 4.2 | 13.3 |
| Secondary Tr1 rest | 3.7 | 21.3 |
| Primary Th1 act | 4.5 | 0.0 |
| Primary Th2 act | 7.4 | 8.8 |
| Primary Tr1 act | 6.1 | 0.0 |
| Primary Th1 rest | 1.4 | 1.7 |
| Primary Th2 rest | 2.8 | 6.6 |
| Primary Tr1 rest | 1.7 | 4.4 |
| CD45RA CD4 lymphocyte act | 6.1 | 10.2 |
| CD45RO CD4 lymphocyte act | 6.7 | 24.7 |
| CD8 lymphocyte act | 8.4 | 0.0 |
| Secondary CD8 lymphocyte rest | 7.0 | 5.2 |
| Secondary CD8 lymphocyte act | 1.2 | 9.9 |
| CD4 lymphocyte none | 6.8 | 1.1 |
| 2ry Th1/Th2/Tr1_anti-CD95 CH11 | 6.6 | 4.9 |
| LAK cells rest | 2.6 | 12.3 |
| LAK cells IL-2 | 3.2 | 9.8 |
| LAK cells IL-2 + IL-12 | 2.2 | 0.0 |
| LAK cells IL-2 + IFN gamma | 2.5 | 0.0 |
| LAK cells IL-2 + IL-18 | 0.9 | 5.8 |
| LAK cells PMA/ionomycin | 2.3 | 8.8 |
| NK Cells IL-2 rest | 12.5 | 25.5 |
| Two Way MLR 3 day | 6.3 | 25.9 |
| Two Way MLR 5 day | 1.8 | 5.0 |
| Two Way MLR 7 day | 4.1 | 7.2 |
| PBMC rest | 5.9 | 6.3 |
| PBMC PWM | 0.0 | 0.0 |
| PBMC PHA-L | 5.8 | 13.3 |
| Ramos (B cell) none | 1.5 | 1.8 |
| Ramos (B cell) ionomycin | 7.8 | 3.3 |
| B lymphocytes PWM | 1.3 | 6.4 |
| B lymphocytes CD40L and IL-4 | 3.6 | 30.4 |
| EOL-1 dbcAMP | 5.4 | 3.0 |
| EOL-1 dbcAMP PMA/ionomycin | 0.0 | 0.0 |
| Dendritic cells none | 6.2 | 8.6 |
| Dendritic cells LPS | 10.7 | 19.8 |
| Dendritic cells anti-CD40 | 3.0 | 12.7 |
| Monocytes rest | 1.2 | 6.6 |
| Monocytes LPS | 27.9 | 100.0 |
| Macrophages rest | 3.2 | 0.0 |
| Macrophages LPS | 10.4 | 28.1 |
| HUVEC none | 0.4 | 0.0 |
| HUVEC starved | 2.8 | 0.0 |
| HUVEC IL-1beta | 1.1 | 0.0 |
| HUVEC IFN gamma | 1.2 | 0.0 |
| HUVEC TNF alpha + IFN gamma | 2.1 | 0.0 |
| HUVEC TNF alpha + IL4 | 1.4 | 0.0 |
| HUVEC IL-11 | 2.8 | 3.6 |
| Lung Microvascular EC none | 3.7 | 0.0 |
| Lung Microvascular EC TNFalpha + | 0.0 | 0.0 |
| IL-1beta | | |
| Microvascular Dermal EC none | 0.6 | 0.0 |
| Microsvasular Dermal EC TNFalpha + | 4.4 | 0.0 |
| IL-1beta | | |
| Bronchial epithelium TNFalpha + | 0.0 | 0.0 |
| IL1beta | | |
| Small airway epithelium none | 34.2 | 0.0 |
| Small airway epithelium TNFalpha + | 25.9 | 9.6 |
| IL-1beta | | |
| Coronery artery SMC rest | 4.8 | 0.0 |
| Coronery artery SMC TNFalpha + IL- | 0.0 | 0.0 |
| 1beta | | |
| Astrocytes rest | 0.7 | 0.0 |
| Astrocytes TNFalpha + IL-1beta | 1.5 | 0.0 |
| KU-812 (Basophil) rest | 5.0 | 3.3 |
| KU-812 (Basophil) PMA/ionomycin | 0.8 | 0.0 |
| CCD1106 (Keratinocytes) none | 5.1 | 6.8 |
| CCD1106 (Keratinocytes) TNFalpha + | 10.0 | 6.6 |
| IL-1beta | | |
| Liver cirrhosis | 6.7 | 3.2 |
| NCI-H292 none | 100.0 | 15.3 |
| NCI-H292 IL-4 | 40.1 | 22.8 |
| NCI-H292 IL-9 | 98.6 | 49.3 |
| NCI-H292 IL-13 | 73.7 | 29.3 |
| NCI-H292 IFN gamma | 49.0 | 15.9 |
| HPAEC none | 8.5 | 0.0 |
| HPAEC TNF alpha + IL-1 beta | 4.7 | 0.0 |
| Lung fibroblast none | 2.8 | 0.0 |
| Lung fibroblast TNF alpha + IL-1 beta | 3.7 | 0.0 |
| Lung fibroblast IL-4 | 2.5 | 0.0 |
| Lung fibroblast IL-9 | 4.1 | 0.0 |
| Lung fibroblast IL-13 | 0.0 | 0.0 |
| Lung fibroblast IFN gamma | 1.4 | 0.0 |
| Dermal fibroblast CCD1070 rest | 1.3 | 0.0 |
| Dermal fibroblast CCD1070 TNF alpha | 9.1 | 21.5 |
| Dermal fibroblast CCD1070 IL-1 beta | 2.8 | 0.0 |
| Dermal fibroblast IFN gamma | 3.1 | 0.0 |
| Dermal fibroblast IL-4 | 2.7 | 0.0 |
| Dermal Fibroblasts rest | 0.3 | 0.0 |
| Neutrophils TNFa + LPS | 1.7 | 2.8 |
| Neutrophils rest | 6.6 | 0.0 |
| Colon | 3.1 | 0.0 |
| Lung | 3.1 | 0.0 |
| Thymus | 0.0 | 0.0 |
| Kidney | 4.5 | 3.4 |

Column A - Rel. Exp. (%) Ag20, Run 268789078
Column B - Rel. Exp. (%) Ag5241, Run 229851725

General_screening_panel_v1.5 Summary: Ag5241 Highest gene expression was detected in T47D breast cancer cell line (CT=30.6) and moderate to low gene expression was detected in melanoma, colon, gastric, gastric, ovarian and breast cancer cell lines. CG50959 gene expression level is a useful marker to differentiate these cancers from normal tissues and to detect the presence of these cancers in vitro or in vivo. Furthermore, therapeutic modulation of this gene is useful in the treatment of these cancers.

Low gene expression was detected in fetal lung and brain. The relative over-expression in fetal tissue indicates that the expressed protein enhances lung and brain growth or development and acts in a regenerative capacity in the adult.

General_screening_panel_v1.6 Summary: Ag20 Highest expression of this gene was detected in T47D breast cancer cell line (CT=26) and moderate to low gene expression was also detected in melanoma, lung, colon, renal, pancreatic, renal, brain, gastric, and breast cancer cell lines. Expression level is a useful marker for differentiating these cancers from normal tissues and to detect the presence of these cancers. Among tissues with metabolic or endocrine function, this gene was expressed at moderate to low levels in pancreas, adipose, thyroid, fetal liver and gastrointestinal tract. Therapeutic modulation of the activity of this gene is useful in the treatment of endocrine/metabolically related diseases, such as obesity and diabetes. Low gene expression levels was detected in the central nervous system including: amygdala, hippocampus, thalamus, cerebellum, and spinal cord. Therefore, therapeutic modulation of this gene and/or expressed protein is useful in the diagnosis and/or treatment of central nervous system disorders such as Alzheimer's disease, Parkinson's disease, epilepsy, multiple sclerosis, schizophrenia and depression.

Panel 4.1D Summary: Ag20 Highest gene expression was seen in resting NCI-H292 (CT=30) and significant expression was detected in activated NCI-H292 cells, small airway epithelium, activated monocytes and macrophages, dendritic cells, and resting IL2 treated NK cells. Ag5241 Highest gene expression using this probe-primer set was detected in LPS activated monocytes (CT=33). Upon activation with pathogens such as LPS, monocytes contribute to innate and specific immunity by migrating to the site of tissue injury and releasing inflammatory cytokines. Modulation of gene expression and/or encoded protein prevents the recruitment of monocytes and the initiation of the inflammatory process, and relieves the symptoms of patients suffering from autoimmune and inflammatory diseases such as asthma, allergies, inflammatory bowel disease, lupus erythematosus, rheumatoid arthritis, or osteoarthritis.

I. CG51018-01: Matrilin-2 Precursor.

Expression of gene CG51018-01 was assessed using the primer-probe set Ag2764, described in Table IA. Results of the RTQ-PCR runs are shown in Tables IB, IC, ID and IE.

TABLE IA

Probe Name Ag2764

| | | | | |
|---|---|---|---|---|
| | | | Start | |
| Primers | Sequences | Length | Position | SEQ ID No |

| | | | | |
|---|---|---|---|---|
| Forward | 5′-tttgcagtgcaacacagatatc-3′ | 22 | 2695 | 388 |
| Probe | TET-5′-ttacggtctacacaaaagctttccca-3′- | 26 | 2737 | 389 |
| | TAMRA | | | |
| Reverse | 5′-gcttcctgaaggttttgttga-3′ | 21 | 2764 | 390 |

TABLE IB
CNS_neurodegeneration_v1.0

| Tissue Name | A |
|---|---|
| AD 1 Hippo | 13.6 |
| AD 2 Hippo | 40.1 |
| AD 3 Hippo | 9.6 |
| AD 4 Hippo | 18.3 |
| AD 5 hippo | 30.1 |
| AD 6 Hippo | 100.0 |
| Control 2 Hippo | 24.3 |
| Control 4 Hippo | 39.5 |
| Control (Path) 3 Hippo | 8.3 |
| AD 1 Temporal Ctx | 21.5 |
| AD 2 Temporal Ctx | 19.1 |
| AD 3 Temporal Ctx | 7.5 |
| AD 4 Temporal Ctx | 22.1 |
| AD 5 Inf Temporal Ctx | 43.2 |
| AD 5 SupTemporal Ctx | 51.8 |
| AD 6 Inf Temporal Ctx | 51.8 |
| AD 6 Sup Temporal Ctx | 61.6 |
| Control 1 Temporal Ctx | 4.2 |
| Control 2 Temporal Ctx | 11.3 |
| Control 3 Temporal Ctx | 6.9 |
| Control 4 Temporal Ctx | 11.3 |
| Control (Path) 1 Temporal Ctx | 33.7 |
| Control (Path) 2 Temporal Ctx | 18.4 |
| Control (Path) 3 Temporal Ctx | 3.8 |
| Control (Path) 4 Temporal Ctx | 18.4 |
| AD 1 Occipital Ctx | 10.4 |
| AD 2 Occipital Ctx (Missing) | 0.0 |
| AD 3 Occipital Ctx | 6.9 |
| AD 4 Occipital Ctx | 14.7 |
| AD 5 Occipital Ctx | 18.3 |
| AD 6 Occipital Ctx | 17.2 |
| Control 1 Occipital Ctx | 2.1 |
| Control 2 Occipital Ctx | 17.7 |
| Control 3 Occipital Ctx | 6.3 |
| Control 4 Occipital Ctx | 15.3 |
| Control (Path) 1 Occipital Ctx | 48.0 |
| Control (Path) 2 Occipital Ctx | 9.2 |
| Control (Path) 3 Occipital Ctx | 1.0 |
| Control (Path) 4 Occipital Ctx | 9.2 |
| Control 1 Parietal Ctx | 5.2 |
| Control 2 Parietal Ctx | 32.5 |
| Control 3 Parietal Ctx | 6.9 |
| Control (Path) 1 Parietal Ctx | 18.8 |
| Control (Path) 2 Parietal Ctx | 25.7 |
| Control (Path) 3 Parietal Ctx | 1.9 |
| Control (Path) 4 Parietal Ctx | 17.1 |

Column A - Rel. Exp. (%) Ag2764, Run 208698723

TABLE IC
Panel 1.3D

| Tissue Name | A |
|---|---|
| Liver adenocarcinoma | 4.9 |
| Pancreas | 1.6 |
| Pancreatic ca. CAPAN 2 | 0.5 |
| Adrenal gland | 6.7 |
| Thyroid | 100.0 |
| Salivary gland | 6.4 |
| Pituitary gland | 3.8 |
| Brain (fetal) | 0.7 |
| Brain (whole) | 3.7 |
| Brain (amygdala) | 8.2 |
| Brain (cerebellum) | 5.3 |
| Brain (hippocampus) | 7.4 |
| Brain (substantia nigra) | 2.5 |
| Brain (thalamus) | 5.4 |
| Cerebral Cortex | 8.1 |
| Spinal cord | 37.1 |
| glio/astro U87-MG | 5.4 |
| glio/astro U-118-MG | 24.5 |
| astrocytoma SW1783 | 33.7 |
| neuro*; met SK-N-AS | 0.1 |
| astrocytoma SF-539 | 13.0 |
| astrocytoma SNB-75 | 7.0 |
| glioma SNB-19 | 0.7 |
| glioma U251 | 5.6 |
| glioma SF-295 | 1.0 |
| Heart (fetal) | 8.8 |
| Heart | 20.0 |
| Skeletal muscle (fetal) | 54.0 |
| Skeletal muscle | 6.9 |
| Bone marrow | 0.9 |
| Thymus | 7.2 |
| Spleen | 2.3 |
| Lymph node | 5.1 |
| Colorectal | 6.7 |
| Stomach | 7.4 |
| Small intestine | 34.6 |
| Colon ca. SW480 | 0.7 |
| Colon ca.* SW620(SW480 met) | 0.1 |
| Colon ca. HT29 | 0.7 |
| Colon ca. HCT-116 | 0.4 |
| Colon ca. CaCo-2 | 1.5 |
| Colon ca. tissue(ODO3866) | 0.9 |
| Colon ca. HCC-2998 | 1.0 |
| Gastric ca.* (liver met) NCI-N87 | 10.0 |
| Bladder | 10.0 |
| Trachea | 26.1 |
| Kidney | 12.3 |
| Kidney (fetal) | 54.3 |
| Renal ca. 786-0 | 1.6 |
| Renal ca. A498 | 6.3 |
| Renal ca. RXF 393 | 14.0 |
| Renal ca. ACHN | 18.4 |
| Renal ca. UO-31 | 13.8 |
| Renal ca. TK-10 | 5.4 |
| Liver | 3.2 |
| Liver (fetal) | 4.6 |
| Liver ca. (hepatoblast) HepG2 | 0.6 |
| Lung | 4.8 |
| Lung (fetal) | 8.2 |
| Lung ca. (small cell) LX-1 | 0.0 |
| Lung ca. (small cell) NCI-H69 | 7.7 |
| Lung ca. (s. cell var.) SHP-77 | 4.8 |
| Lung ca. (large cell)NCI-H460 | 0.0 |
| Lung ca. (non-sm. cell) A549 | 1.4 |
| Lung ca. (non-s. cell) NCI-H23 | 3.4 |
| Lung ca. (non-s. cell) HOP-62 | 0.9 |
| Lung ca. (non-s. cl) NCI-H522 | 0.5 |
| Lung ca. (squam.) SW 900 | 0.4 |
| Lung ca. (squam.) NCI-H596 | 3.3 |
| Mammary gland | 29.7 |
| Breast ca.* (pl. ef) MCF-7 | 6.0 |
| Breast ca.* (pl. ef) MDA-MB-231 | 6.3 |
| Breast ca.* (pl. ef) T47D | 1.8 |
| Breast ca. BT-549 | 7.4 |
| Breast ca. MDA-N | 0.0 |
| Ovary | 65.5 |
| Ovarian ca. OVCAR-3 | 0.9 |
| Ovarian ca. OVCAR-4 | 0.1 |
| Ovarian ca. OVCAR-5 | 2.0 |
| Ovarian ca. OVCAR-8 | 16.3 |
| Ovarian ca. IGROV-1 | 0.6 |
| Ovarian ca.* (ascites) SK-OV-3 | 1.1 |
| Uterus | 55.1 |
| Placenta | 14.8 |
| Prostate | 20.3 |
| Prostate ca.* (bone met)PC-3 | 5.5 |
| Testis | 13.2 |
| Melanoma Hs688(A).T | 0.9 |
| Melanoma* (met) Hs688(B).T | 0.8 |
| Melanoma UACC-62 | 0.1 |
| Melanoma M14 | 0.6 |
| Melanoma LOX IMVI | 0.1 |
| Melanoma* (met) SK-MEL-5 | 0.8 |
| Adipose | 13.2 |

Column A - Rel. Exp. (%) Ag2764, Run 164024018

TABLE ID
Panel 2D

| Tissue Name | A |
|---|---|
| Normal Colon | 21.2 |
| CC Well to Mod Diff (ODO3866) | 0.3 |
| CC Margin (ODO3866) | 4.1 |
| CC Gr.2 rectosigmoid (ODO3868) | 0.5 |
| CC Margin (ODO3868) | 2.2 |
| CC Mod Diff (ODO3920) | 1.0 |
| CC Margin (ODO3920) | 5.7 |
| CC Gr.2 ascend colon (ODO3921) | 4.3 |
| CC Margin (ODO3921) | 4.4 |
| CC from Partial Hepatectomy (ODO4309) Mets | 1.0 |
| Liver Margin (ODO4309) | 3.8 |
| Colon mets to lung (OD04451-01) | 0.4 |
| Lung Margin (OD04451-02) | 0.4 |
| Normal Prostate 6546-1 | 77.4 |
| Prostate Cancer (OD04410) | 15.1 |
| Prostate Margin (OD04410) | 21.3 |
| Prostate Cancer (OD04720-01) | 18.3 |
| Prostate Margin (OD04720-02) | 23.8 |
| Normal Lung 061010 | 5.0 |
| Lung Met to Muscle (ODO4286) | 0.4 |
| Muscle Margin (ODO4286) | 0.6 |
| Lung Malignant Cancer (OD03126) | 1.1 |
| Lung Margin (OD03126) | 1.6 |
| Lung Cancer (OD04404) | 2.2 |
| Lung Margin (OD04404) | 2.1 |
| Lung Cancer (OD04565) | 3.1 |
| Lung Margin (OD04565) | 0.8 |
| Lung Cancer (OD04237-01) | 1.0 |
| Lung Margin (OD04237-02) | 2.4 |
| Ocular Mel Met to Liver (ODO4310) | 9.7 |
| Liver Margin (ODO4310) | 3.6 |
| Melanoma Mets to Lung (OD04321) | 2.2 |
| Lung Margin (OD04321) | 1.5 |
| Normal Kidney | 16.7 |
| Kidney Ca, Nuclear grade 2 (OD04338) | 2.7 |
| Kidney Margin (OD04338) | 9.2 |
| Kidney Ca Nuclear grade 1/2 (OD04339) | 0.6 |
| Kidney Margin (OD04339) | 7.5 |
| Kidney Ca, Clear cell type (OD04340) | 9.3 |
| Kidney Margin (OD04340) | 8.5 |
| Kidney Ca, Nuclear grade 3 (OD04348) | 0.8 |
| Kidney Margin (OD04348) | 5.8 |
| Kidney Cancer (OD04622-01) | 0.6 |
| Kidney Margin (OD04622-03) | 1.0 |
| Kidney Cancer (OD04450-01) | 6.9 |
| Kidney Margin (OD04450-03) | 8.2 |
| Kidney Cancer 8120607 | 1.9 |
| Kidney Margin 8120608 | 1.3 |
| Kidney Cancer 8120613 | 0.2 |
| Kidney Margin 8120614 | 1.0 |
| Kidney Cancer 9010320 | 0.9 |
| Kidney Margin 9010321 | 1.2 |
| Normal Uterus | 12.2 |
| Uterus Cancer 064011 | 17.4 |
| Normal Thyroid | 100.0 |
| Thyroid Cancer 064010 | 6.8 |
| Thyroid Cancer A302152 | 3.6 |
| Thyroid Margin A302153 | 39.8 |
| Normal Breast | 9.1 |
| Breast Cancer (OD04566) | 2.1 |
| Breast Cancer (OD04590-01) | 2.0 |
| Breast Cancer Mets (OD04590-03) | 4.4 |
| Breast Cancer Metastasis (OD04655-05) | 2.0 |
| Breast Cancer 064006 | 1.5 |
| Breast Cancer 1024 | 3.6 |
| Breast Cancer 9100266 | 2.1 |
| Breast Margin 9100265 | 4.9 |
| Breast Cancer A209073 | 6.3 |
| Breast Margin A209073 | 8.5 |
| Normal Liver | 3.5 |
| Liver Cancer 064003 | 0.8 |
| Liver Cancer 1025 | 1.4 |
| Liver Cancer 1026 | 0.4 |
| Liver Cancer 6004-T | 1.2 |
| Liver Tissue 6004-N | 0.2 |
| Liver Cancer 6005-T | 0.6 |
| Liver Tissue 6005-N | 0.3 |
| Normal Bladder | 4.3 |
| Bladder Cancer 1023 | 0.5 |
| Bladder Cancer A302173 | 1.9 |
| Bladder Cancer (OD04718-01) | 6.7 |
| Bladder Normal Adjacent (OD04718-03) | 8.2 |
| Normal Ovary | 6.4 |
| Ovarian Cancer 064008 | 7.2 |
| Ovarian Cancer (OD04768-07) | 4.4 |
| Ovary Margin (OD04768-08) | 3.9 |
| Normal Stomach | 6.7 |
| Gastric Cancer 9060358 | 1.7 |
| Stomach Margin 9060359 | 2.1 |
| Gastric Cancer 9060395 | 7.2 |
| Stomach Margin 9060394 | 3.3 |
| Gastric Cancer 9060397 | 1.6 |
| Stomach Margin 9060396 | 0.4 |
| Gastric Cancer 064005 | 6.6 |

Column A - Rel. Exp. (%) Ag2764, Run 162556852

TABLE IE
Panel 5 Islet

| Tissue Name | A |
|---|---|
| 97457_Patient-02go_adipose | 17.6 |
| 97476_Patient-07sk_skeletal muscle | 19.2 |
| 97477_Patient-07ut_uterus | 45.4 |
| 97478_Patient-07pl_placenta | 15.5 |
| 99167_Bayer Patient 1 | 1.0 |
| 97482_Patient-08ut_uterus | 21.5 |
| 97483_Patient-08pl_placenta | 16.3 |
| 97486_Patient-09sk_skeletal muscle | 2.2 |
| 97487_Patient-09ut_uterus | 92.7 |
| 97488_Patient-09pl_placenta | 12.0 |
| 97492_Patient-10ut_uterus | 50.3 |
| 97493_Patient-10pl_placenta | 21.5 |
| 97495_Patient-11go_adipose | 12.2 |
| 97496_Patient-11sk_skeletal muscle | 2.9 |
| 97497_Patient-11ut_uterus | 100.0 |
| 97498_Patient-11pl_placenta | 6.1 |
| 97500_Patient-12go_adipose | 15.2 |
| 97501_Patient-12sk_skeletal muscle | 9.2 |
| 97502_Patient-12ut_uterus | 70.2 |
| 97503_Patient-12pl_placenta | 5.3 |
| 94721_Donor 2 U - A_Mesenchymal Stem Cells | 3.5 |
| 94722_Donor 2 U - B_Mesenchymal Stem Cells | 2.5 |
| 94723_Donor 2 U - C_Mesenchymal Stem Cells | 2.5 |
| 94709_Donor 2 AM - A_adipose | 1.4 |
| 94710_Donor 2 AM - B_adipose | 0.4 |
| 94711_Donor 2 AM - C_adipose | 0.7 |
| 94712_Donor 2 AD - A_adipose | 6.0 |
| 94713_Donor 2 AD - B_adipose | 8.1 |
| 94714_Donor 2 AD - C_adipose | 12.4 |
| 94742_Donor 3 U - A_Mesenchymal Stem Cells | 1.4 |
| 94743_Donor 3 U - B_Mesenchymal Stem Cells | 3.0 |
| 94730_Donor 3 AM - A_adipose | 1.8 |
| 94731_Donor 3 AM - B_adipose | 1.1 |
| 94732_Donor 3 AM - C_adipose | 1.2 |
| 94733_Donor 3 AD - A_adipose | 2.8 |
| 94734_Donor 3 AD - B_adipose | 2.6 |
| 94735_Donor 3 AD - C_adipose | 4.7 |
| 77138_Liver_HepG2untreated | 0.5 |
| 73556_Heart_Cardiac stromal cells (primary) | 0.6 |
| 81735_Small Intestine | 20.6 |
| 72409_Kidney_Proximal Convoluted Tubule | 1.2 |
| 82685_Small intestine_Duodenum | 11.3 |
| 90650_Adrenal_Adrenocortical adenoma | 2.0 |
| 72410_Kidney_HRCE | 0.6 |
| 72411_Kidney_HRE | 0.9 |
| 73139_Uterus_Uterine smooth muscle cells | 2.8 |

Column A - Rel. Exp. (%) Ag2764, Run 254275033

CNS_neurodegeneration_v1.0 Summary: Ag2764 This gene, a homolog of matrilin-2, is an intercellular matrix protein. The results of this panel shows expression in the brain. Glial scarring is a major inhibitor of CNS repair/regeneration involving intra and extra-cellular proteins. Reduction of expression levels of this gene or protein encoded by this gene decreases glial scarring in response to CNS injury, and promotes healing in spinal cord and/or brain trauma.

Panel 1.3D Summary: Ag2764 High gene expressed was detected in the thyroid gland (CT=26.2), fetal kidney and fetal skeletal muscle (CTs=27.1) and shows an association with normal tissue when compared to cancer cell lines. This gene was moderately expressed in pancreas, adrenal and pituitary glands, adipose, fetal and adult heart, fetal and adult liver, and adult skeletal muscle. The relative over expression of this gene in fetal skeletal muscle relative to adult skeletal muscle indicates that the protein product enhances muscular growth or development and acts in a regenerative capacity in the adult. Modulation of gene expression is useful in treatment of muscle related diseases treatment of weak or dystrophic muscle with the encoded protein restores muscle mass or function.

This gene is expressed in many tissues of the central nervous system including: amygdala, cerebellum, hippocampus, substantia nigra, thalamus, cerebral cortex, spinal cord, and the developing brain.

Panel 2D Summary: Ag2764 Highest gene expression was detected in normal thyroid tissue (CT=24.7) and there was a strong association of gene expression in normal prostate tissue (CT=25). This gene was overexpressed in normal thyroid tissue relative to samples derived from matched thyroid cancer tissue. Thus, therapeutic modulation of the activity or expression of this gene, encoded protein, and/or antibodies, small molecule drugs targeting the encoded protein is an effective treatment of thyroid and prostate cancers.

Panel 5 Islet Summary: Ag2764 Highest expression of this gene was seen in uterus of a non-diabetic but overweight patient (CT=25.9). High gene expression was seen in uterus, adipose, skeletal muscle, placenta, kidney and small intestine and moderate expression was seen in islet cells. Therefore, therapeutic modulation of this gene and/or encoded protein is useful in the treatment of metabolic/endocrine diseases including obesity and diabetes.

J. CG51051-07 and CG51051-09: Netrin GID Like.

Expression of gene CG51051-07 and CG51051-09 was assessed using the primer-probe sets Ag290 and Ag040, described in Tables JA and JB. Results of the RTQ-PCR runs are shown in Tables JC and JD.

TABLE JA

Probe Name Ag290

| | | | | |
|---|---|---|---|---|
| | | | Start | |
| Primers | Sequences | Length | Position | SEQ ID No |

| | | | | |
|---|---|---|---|---|
| Forward | 5′-ggcacgtccctccgttct-3′ | 18 | 1292 | 391 |
| Probe | TET-5′-ctgcgacaacgagctcctgcactg-3′ | 24 | 1266 | 392 |
| | TAMRA | | | |
| Reverse | 5′-ctgttcaagttgcaaaccacaag-3′ | 23 | 1232 | 393 |

TABLE JB
Probe Name Ag040

| | | | Start | SEQ ID |
|---|---|---|---|---|
| Primers | Sequences | Length | Position | No |
| Forward | 5′-ggcacgtccctccgttct-3′ | 18 | 1292 | 394 |
| Probe | TET-5′-ctgcgacaacgagctcctgcactg-3′-TAMRA | 24 | 1266 | 395 |
| Reverse | 5′-ctgttcaagttgcaaaccacaag-3′ | 23 | 1232 | 396 |

TABLE JC
CNS_neurodegeneration_v1.0

| Tissue Name | A | B |
|---|---|---|
| AD 1 Hippo | 2.3 | 4.2 |
| AD 2 Hippo | 18.6 | 38.7 |
| AD 3 Hippo | 1.4 | 4.0 |
| AD 4 Hippo | 5.7 | 8.3 |
| AD 5 hippo | 100.0 | 100.0 |
| AD 6 Hippo | 4.9 | 14.2 |
| Control 2 Hippo | 11.4 | 23.0 |
| Control 4 Hippo | 2.5 | 2.7 |
| Control (Path) 3 Hippo | 0.7 | 2.8 |
| AD 1 Temporal Ctx | 1.2 | 3.8 |
| AD 2 Temporal Ctx | 7.8 | 14.5 |
| AD 3 Temporal Ctx | 0.4 | 4.4 |
| AD 4 Temporal Ctx | 4.6 | 5.8 |
| AD 5 Inf Temporal Ctx | 31.2 | 44.4 |
| AD 5 Sup Temporal Ctx | 20.0 | 18.0 |
| AD 6 Inf Temporal Ctx | 6.3 | 16.4 |
| AD 6 Sup Temporal Ctx | 9.5 | 24.7 |
| Control 1 Temporal Ctx | 2.4 | 3.8 |
| Control 2 Temporal Ctx | 14.1 | 20.2 |
| Control 3 Temporal Ctx | 4.2 | 6.6 |
| Control 4 Temporal Ctx | 0.7 | 5.4 |
| Control (Path) 1 Temporal Ctx | 23.0 | 50.3 |
| Control (Path) 2 Temporal Ctx | 14.3 | 42.0 |
| Control (Path) 3 Temporal Ctx | 2.2 | 5.3 |
| Control (Path) 4 Temporal Ctx | 16.3 | 23.2 |
| AD 1 Occipital Ctx | 8.5 | 19.6 |
| AD 2 Occipital Ctx (Missing) | 0.0 | 0.0 |
| AD 3 Occipital Ctx | 2.7 | 3.2 |
| AD 4 Occipital Ctx | 6.9 | 14.9 |
| AD 5 Occipital Ctx | 6.6 | 39.8 |
| AD 6 Occipital Ctx | 45.1 | 13.6 |
| Control 1 Occipital Ctx | 3.0 | 1.6 |
| Control 2 Occipital Ctx | 30.4 | 74.2 |
| Control 3 Occipital Ctx | 9.9 | 24.0 |
| Control 4 Occipital Ctx | 2.8 | 8.4 |
| Control (Path) 1 Occipital Ctx | 39.8 | 90.1 |
| Control (Path) 2 Occipital Ctx | 10.4 | 21.6 |
| Control (Path) 3 Occipital Ctx | 1.2 | 7.9 |
| Control (Path) 4 Occipital Ctx | 11.0 | 29.3 |
| Control 1 Parietal Ctx | 1.7 | 6.9 |
| Control 2 Parietal Ctx | 11.9 | 22.7 |
| Control 3 Parietal Ctx | 7.2 | 9.7 |
| Control (Path) 1 Parietal Ctx | 18.9 | 62.0 |
| Control (Path) 2 Parietal Ctx | 6.4 | 14.1 |
| Control (Path) 3 Parietal Ctx | 0.5 | 1.6 |
| Control (Path) 4 Parietal Ctx | 15.7 | 52.5 |

Column A - Rel. Exp. (%) Ag040, Run 206975031
Column B - Rel. Exp. (%) Ag040, Run 269217520

TABLE JD
Panel 1

| Tissue Name | A |
|---|---|
| Endothelial cells | 0.0 |
| Endothelial cells (treated) | 0.0 |
| Pancreas | 12.2 |
| Pancreatic ca. CAPAN 2 | 1.7 |
| Adrenal gland | 0.2 |
| Thyroid | 0.0 |
| Salivary gland | 0.1 |
| Pituitary gland | 0.0 |
| Brain (fetal) | 0.0 |
| Brain (whole) | 8.0 |
| Brain (amygdala) | 0.0 |
| Brain (cerebellum) | 49.3 |
| Brain (hippocampus) | 0.0 |
| Brain (substantia nigra) | 0.0 |
| Brain (thalamus) | 0.0 |
| Brain (hypothalamus) | 0.0 |
| Spinal cord | 0.0 |
| glio/astro U87-MG | 0.0 |
| glio/astro U-118-MG | 0.0 |
| astrocytoma SW1783 | 0.0 |
| neuro*; met SK-N-AS | 0.0 |
| astrocytoma SF-539 | 0.0 |
| astrocytoma SNB-75 | 0.0 |
| glioma SNB-19 | 0.5 |
| glioma U251 | 0.0 |
| glioma SF-295 | 3.2 |
| Heart | 0.0 |
| Skeletal muscle | 0.0 |
| Bone marrow | 8.0 |
| Thymus | 100.0 |
| Spleen | 3.3 |
| Lymph node | 7.3 |
| Colon (ascending) | 10.7 |
| Stomach | 58.6 |
| Small intestine | 0.8 |
| Colon ca. SW480 | 0.0 |
| Colon ca.* SW620 (SW480 met) | 0.0 |
| Colon ca. HT29 | 0.0 |
| Colon ca. HCT-116 | 0.1 |
| Colon ca. CaCo-2 | 0.9 |
| Colon ca. HCT-15 | 0.0 |
| Colon ca. HCC-2998 | 0.0 |
| Gastric ca. * (liver met) NCI-N87 | 3.7 |
| Bladder | 0.0 |
| Trachea | 28.1 |
| Kidney | 0.1 |
| Kidney (fetal) | 3.0 |
| Renal ca. 786-0 | 0.0 |
| Renal ca. A498 | 0.0 |
| Renal ca. RXF 393 | 0.0 |
| Renal ca. ACHN | 0.0 |
| Renal ca. UO-31 | 0.0 |
| Renal ca. TK-10 | 1.1 |
| Liver | 59.9 |
| Liver (fetal) | 0.1 |
| Liver ca. (hepatoblast) HepG2 | 0.0 |
| Lung | 0.1 |
| Lung (fetal) | 0.0 |
| Lung ca. (small cell) LX-1 | 0.0 |
| Lung ca. (small cell) NCI-H69 | 0.0 |
| Lung ca. (s. cell var.) SHP-77 | 0.0 |
| Lung ca. (large cell)NCI-H460 | 0.0 |
| Lung ca. (non-sm. cell) A549 | 0.0 |
| Lung ca. (non-s. cell) NCI-H23 | 0.0 |
| Lung ca. (non-s. cell) HOP-62 | 0.0 |
| Lung ca. (non-s. cl) NCI-H522 | 0.0 |
| Lung ca. (squam.) SW 900 | 0.0 |
| Lung ca. (squam.) NCI-H596 | 1.6 |
| Mammary gland | 95.3 |
| Breast ca.* (pl. ef) MCF-7 | 17.1 |
| Breast ca.* (pl. ef) MDA-MB-231 | 0.0 |
| Breast ca.* (pl. ef) T47D | 0.0 |
| Breast ca. BT-549 | 0.0 |
| Breast ca. MDA-N | 0.0 |
| Ovary | 0.0 |
| Ovarian ca. OVCAR-3 | 0.0 |
| Ovarian ca. OVCAR-4 | 0.0 |
| Ovarian ca. OVCAR-5 | 1.3 |
| Ovarian ca. OVCAR-8 | 0.0 |
| Ovarian ca. IGROV-1 | 0.0 |
| Ovarian ca. (ascites) SK-OV-3 | 0.0 |
| Uterus | 0.3 |
| Placenta | 9.9 |
| Prostate | 12.2 |
| Prostate ca.* (bone met) PC-3 | 0.0 |
| Testis | 90.8 |
| Melanoma Hs688(A).T | 0.0 |
| Melanoma* (met) Hs688(B).T | 0.0 |
| Melanoma UACC-62 | 0.0 |
| Melanoma M14 | 0.0 |
| Melanoma LOX IMVI | 0.0 |
| Melanoma* (met) SK-MEL-5 | 0.0 |
| Melanoma SK-MEL-28 | 0.0 |

Column A - Rel. Exp. (%) Ag290, Run 87988084

CNS_neurodegeneration_v1.0 Summary: Ag040 This gene was downregulated in the temporal cortex of Alzheimer's diseased brain as analyzed by ANCOVA, with RNA quality as a covariate. This gene codes for Netrin like protein. Netrins are secreted proteins which have both neurotrophic and neuroprotective functions. They are believed to play a role in neurodevelopment, both in neuron survival and in axon guidance (Manitt C, Kennedy T E., 2002,Where the rubber meets the road: netrin expression and function in developing and adult nervous systems. Prog Brain Res. 137:425-42;PMID: 12440385). Therefore, up-regulation of this gene or its protein product, is useful preventing, treating or ameliorating symptoms associated with this disease.

Panel 1 Summary: Ag290 Highest gene expression was seen in thymus (CT=26). Indicating that this gene plays a role in T cell development. Targeting the encoded protein is useful for modulating immune function (T cell development) and is important for organ transplantation, AIDS treatment or post chemotherapy immune reconstitiution. Significant gene expression was seen in tissues with metabolic/endocrine function including pancreas, liver and gastrointestinal tract. Therapeutic gene and/or encoded protein modulation is useful in the treatment of metabolic/endocrine diseases including diabetes and obesity. High gene expression was detected in whole brain and cerebellum. Therefore, therapeutic modulation of this gene and/or encoded protein is useful in the treatment of neurological disorders such as ataxia, and autism.

K. CG51051-14: Netrin GID Like.

Expression of gene CG51051-14 was assessed using the primer-probe set Ag6679, described in Table KA. Results of the RTQ-PCR runs are shown in Tables KB, KC and KD. Table KA. Probe Name Ag6679

TABLE KA

Probe Name Ag6679

| | | | | |
|---|---|---|---|---|
| Primers | Sequences | Length | Start Position | SEQ ID No |

| | | | | |
|---|---|---|---|---|
| Forward | 5′-ccagtattggtacgaatgtctg-3′ | 22 | 228 | 397 |
| Probe | TET-5′-ctcctgcactgccagaacggag-3′-TAMRA | 22 | 196 | 398 |
| Reverse | 5′-acaggcagcgcacgt-3′ | 15 | 166 | 399 |

TABLE KB
CNS_neurodegeneration_v1.0

| Tissue Name | A |
|---|---|
| AD 1 Hippo | 4.5 |
| AD 2 Hippo | 26.4 |
| AD 3 Hippo | 7.1 |
| AD 4 Hippo | 8.1 |
| AD 5 hippo | 67.4 |
| AD 6 Hippo | 34.2 |
| Control 2 Hippo | 22.4 |
| Control 4 Hippo | 9.0 |
| Control (Path) 3 Hippo | 2.3 |
| AD 1 Temporal Ctx | 17.2 |
| AD 2 Temporal Ctx | 13.6 |
| AD 3 Temporal Ctx | 0.0 |
| AD 4 Temporal Ctx | 8.3 |
| AD 5 Inf Temporal Ctx | 24.0 |
| AD 5 SupTemporal Ctx | 27.9 |
| AD 6 Inf Temporal Ctx | 11.3 |
| AD 6 Sup Temporal Ctx | 31.0 |
| Control 1 Temporal Ctx | 1.4 |
| Control 2 Temporal Ctx | 31.9 |
| Control 3 Temporal Ctx | 6.4 |
| Control 4 Temporal Ctx | 1.4 |
| Control (Path) 1 Temporal Ctx | 36.1 |
| Control (Path) 2 Temporal Ctx | 38.4 |
| Control (Path) 3 Temporal Ctx | 1.3 |
| Control (Path) 4 Temporal Ctx | 17.0 |
| AD 1 Occipital Ctx | 17.0 |
| AD 2 Occipital Ctx (Missing) | 0.0 |
| AD 3 Occipital Ctx | 2.9 |
| AD 4 Occipital Ctx | 7.3 |
| AD 5 Occipital Ctx | 20.3 |
| AD 6 Occipital Ctx | 35.1 |
| Control 1 Occipital Ctx | 2.0 |
| Control 2 Occipital Ctx | 71.7 |
| Control 3 Occipital Ctx | 17.4 |
| Control 4 Occipital Ctx | 1.8 |
| Control (Path) 1 Occipital Ctx | 100.0 |
| Control (Path) 2 Occipital Ctx | 4.5 |
| Control (Path) 3 Occipital Ctx | 0.9 |
| Control (Path) 4 Occipital Ctx | 15.3 |
| Control 1 Parietal Ctx | 5.7 |
| Control 2 Parietal Ctx | 11.5 |
| Control 3 Parietal Ctx | 8.6 |
| Control (Path) 1 Parietal Ctx | 34.6 |
| Control (Path) 2 Parietal Ctx | 12.8 |
| Control (Path) 3 Parietal Ctx | 2.3 |
| Control (Path) 4 Parietal Ctx | 33.0 |

Column A - Rel. Exp. (%) Ag6679, Run 275777964

TABLE KC
General_screening_panel_v1.6

| Tissue Name | A |
|---|---|
| Adipose | 3.0 |
| Melanoma* Hs688(A).T | 2.7 |
| Melanoma* Hs688(B).T | 1.3 |
| Melanoma* M14 | 0.0 |
| Melanoma* LOXIMVI | 15.0 |
| Melanoma* SK-MEL-5 | 0.0 |
| Squamous cell carcinoma SCC-4 | 1.3 |
| Testis Pool | 0.2 |
| Prostate ca.* (bone met) PC-3 | 1.2 |
| Prostate Pool | 2.7 |
| Placenta | 0.3 |
| Uterus Pool | 0.8 |
| Ovarian ca. OVCAR-3 | 3.3 |
| Ovarian ca. SK-OV-3 | 66.9 |
| Ovarian ca. OVCAR-4 | 0.3 |
| Ovarian ca. OVCAR-5 | 0.0 |
| Ovarian ca. IGROV-1 | 0.0 |
| Ovarian ca. OVCAR-8 | 8.2 |
| Ovary | 0.0 |
| Breast ca. MCF-7 | 0.0 |
| Breast ca. MDA-MB-231 | 0.0 |
| Breast ca. BT 549 | 10.6 |
| Breast ca. T47D | 0.0 |
| Breast ca. MDA-N | 0.0 |
| Breast Pool | 1.0 |
| Trachea | 0.5 |
| Lung | 0.0 |
| Fetal Lung | 35.1 |
| Lung ca. NCI-N417 | 10.7 |
| Lung ca. LX-1 | 0.0 |
| Lung ca. NCI-H146 | 27.2 |
| Lung ca. SHP-77 | 4.6 |
| Lung ca. A549 | 0.0 |
| Lung ca. NCI-H526 | 0.0 |
| Lung ca. NCI-H23 | 0.0 |
| Lung ca. NCI-H460 | 0.2 |
| Lung ca. HOP-62 | 0.3 |
| Lung ca. NCI-H522 | 0.0 |
| Liver | 0.0 |
| Fetal Liver | 0.2 |
| Liver ca. HepG2 | 0.0 |
| Kidney Pool | 0.0 |
| Fetal Kidney | 31.6 |
| Renal ca. 786-0 | 8.1 |
| Renal ca. A498 | 5.5 |
| Renal ca. ACHN | 3.0 |
| Renal ca. UO-31 | 9.5 |
| Renal ca. TK-10 | 4.0 |
| Bladder | 0.4 |
| Gastric ca. (liver met.) NCI-N87 | 0.0 |
| Gastric ca. KATO III | 0.0 |
| Colon ca. SW-948 | 0.0 |
| Colon ca. SW480 | 0.0 |
| Colon ca.* (SW480 met) SW620 | 0.0 |
| Colon ca. HT29 | 0.0 |
| Colon ca. HCT-116 | 0.0 |
| Colon ca. CaCo-2 | 0.1 |
| Colon cancer tissue | 0.4 |
| Colon ca. SW1116 | 0.0 |
| Colon ca. Colo-205 | 0.0 |
| Colon ca. SW-48 | 0.0 |
| Colon Pool | 0.1 |
| Small Intestine Pool | 1.4 |
| Stomach Pool | 0.7 |
| Bone Marrow Pool | 1.2 |
| Fetal Heart | 0.8 |
| Heart Pool | 0.2 |
| Lymph Node Pool | 1.0 |
| Fetal Skeletal Muscle | 2.3 |
| Skeletal Muscle Pool | 0.0 |
| Spleen Pool | 0.0 |
| Thymus Pool | 0.6 |
| CNS cancer (glio/astro) U87-MG | 15.2 |
| CNS cancer (glio/astro) U-118-MG | 100.0 |
| CNS cancer (neuro; met) SK-N-AS | 1.3 |
| CNS cancer (astro) SF-539 | 1.1 |
| CNS cancer (astro) SNB-75 | 27.9 |
| CNS cancer (glio) SNB-19 | 0.0 |
| CNS cancer (glio) SF-295 | 0.0 |
| Brain (Amygdala) Pool | 7.8 |
| Brain (cerebellum) | 5.7 |
| Brain (fetal) | 3.7 |
| Brain (Hippocampus) Pool | 3.5 |
| Cerebral Cortex Pool | 5.0 |
| Brain (Substantia nigra) Pool | 1.5 |
| Brain (Thalamus) Pool | 21.5 |
| Brain (whole) | 10.2 |
| Spinal Cord Pool | 4.2 |
| Adrenal Gland | 1.6 |
| Pituitary gland Pool | 1.3 |
| Salivary Gland | 0.1 |
| Thyroid (female) | 0.2 |
| Pancreatic ca. CAPAN2 | 0.0 |
| Pancreas Pool | 0.3 |

Column A - Rel. Exp. (%) Ag6679, Run 277244468

TABLE KD
Panel 4.1D

| Tissue Name | A |
|---|---|
| Secondary Th1 act | 0.0 |
| Secondary Th2 act | 0.0 |
| Secondary Tr1 act | 0.0 |
| Secondary Th1 rest | 0.0 |
| Secondary Th2 rest | 0.0 |
| Secondary Tr1 rest | 0.0 |
| Primary Th1 act | 0.0 |
| Primary Th2 act | 0.0 |
| Primary Tr1 act | 0.0 |
| Primary Th1 rest | 0.0 |
| Primary Th2 rest | 0.0 |
| Primary Tr1 rest | 0.0 |
| CD45RA CD4 lymphocyte act | 17.1 |
| CD45RO CD4 lymphocyte act | 0.0 |
| CD8 lymphocyte act | 0.0 |
| Secondary CD8 lymphocyte rest | 0.0 |
| Secondary CD8 lymphocyte act | 0.0 |
| CD4 lymphocyte none | 0.0 |
| 2ry Th1/Th2/Tr1_anti-CD95 CH11 | 0.0 |
| LAK cells rest | 0.0 |
| LAK cells IL-2 | 0.0 |
| LAK cells IL-2 + IL-12 | 0.0 |
| LAK cells IL-2 + IFN gamma | 0.0 |
| LAK cells IL-2 + IL-18 | 0.0 |
| LAK cells PMA/ionomycin | 5.5 |
| NK Cells IL-2 rest | 4.5 |
| Two Way MLR 3 day | 0.0 |
| Two Way MLR 5 day | 0.0 |
| Two Way MLR 7 day | 0.0 |
| PBMC rest | 0.0 |
| PBMC PWM | 0.0 |
| PBMC PHA-L | 0.0 |
| Ramos (B cell) none | 0.0 |
| Ramos (B cell) ionomycin | 0.0 |
| B lymphocytes PWM | 0.0 |
| B lymphocytes CD40L and IL-4 | 4.8 |
| EOL-1 dbcAMP | 0.0 |
| EOL-1 dbcAMP PMA/ionomycin | 0.0 |
| Dendritic cells none | 0.0 |
| Dendritic cells LPS | 0.0 |
| Dendritic cells anti-CD40 | 0.0 |
| Monocytes rest | 0.0 |
| Monocytes LPS | 0.0 |
| Macrophages rest | 0.0 |
| Macrophages LPS | 0.0 |
| HUVEC none | 0.0 |
| HUVEC starved | 0.0 |
| HUVEC IL-1beta | 0.0 |
| HUVEC IFN gamma | 0.0 |
| HUVEC TNF alpha + IFN gamma | 0.0 |
| HUVEC TNF alpha + IL4 | 0.0 |
| HUVEC IL-11 | 0.0 |
| Lung Microvascular EC none | 7.4 |
| Lung Microvascular EC TNFalpha + IL-1beta | 0.0 |
| Microvascular Dermal EC none | 0.0 |
| Microsvasular Dermal EC TNFalpha + IL-1beta | 0.0 |
| Bronchial epithelium TNFalpha + IL1beta | 10.7 |
| Small airway epithelium none | 8.7 |
| Small airway epithelium TNFalpha + IL-1beta | 0.0 |
| Coronery artery SMC rest | 0.0 |
| Coronery artery SMC TNFalpha + IL-1beta | 0.0 |
| Astrocytes rest | 34.9 |
| Astrocytes TNFalpha + IL-1beta | 4.2 |
| KU-812 (Basophil) rest | 0.0 |
| KU-812 (Basophil) PMA/ionomycin | 0.0 |
| CCD1106 (Keratinocytes) none | 12.2 |
| CCD1106 (Keratinocytes) TNFalpha + IL-1beta | 13.1 |
| Liver cirrhosis | 0.0 |
| NCI-H292 none | 0.0 |
| NCI-H292 IL-4 | 0.0 |
| NCI-H292 IL-9 | 0.0 |
| NCI-H292 IL-13 | 0.0 |
| NCI-H292 IFN gamma | 0.0 |
| HPAEC none | 8.8 |
| HPAEC TNF alpha + IL-1 beta | 0.0 |
| Lung fibroblast none | 0.0 |
| Lung fibroblast TNF alpha + IL-1 beta | 0.0 |
| Lung fibroblast IL-4 | 0.0 |
| Lung fibroblast IL-9 | 0.0 |
| Lung fibroblast IL-13 | 0.0 |
| Lung fibroblast IFN gamma | 0.0 |
| Dermal fibroblast CCD1070 rest | 56.3 |
| Dermal fibroblast CCD1070 TNF alpha | 40.6 |
| Dermal fibroblast CCD1070 IL-1 beta | 65.1 |
| Dermal fibroblast IFN gamma | 24.5 |
| Dermal fibroblast IL-4 | 15.3 |
| Dermal Fibroblasts rest | 56.6 |
| Neutrophils TNFa + LPS | 0.0 |
| Neutrophils rest | 0.0 |
| Colon | 0.0 |
| Lung | 7.0 |
| Thymus | 0.0 |
| Kidney | 100.0 |

Column A - Rel. Exp. (%) Ag6679, Run 275769774

CNS_neurodegeneration_v1.0 Summary: Ag6679 This gene was downregulated in the temporal cortex of Alzheimer's diseased brain as analyzed by ANCOVA, with RNA quality as a covariate. This gene codes for Netrin like protein. Netrins are secreted proteins which have both neurotrophic and neuroprotective functions. They are believed to play a role in neurodevelopment, both in neuron survival and in axon guidance (Manitt C, Kennedy T E., 2002,Where the rubber meets the road: netrin expression and function in developing and adult nervous systems. Prog Brain Res. 137:425-42;PMID: 12440385). Therefore, up-regulation of this gene or its protein product, is useful preventing, treating or ameliorating symptoms associated with this disease.

General_screening_panel_v1.6 Summary: Ag6679 Highest CG51051-14 gene expression was seen in U-118-MG brain cancer cell line (CT=28.1) and moderate to low gene expression was detected in melanoma, ovarian, breast, lung, renal and brain cancer cell lines. Gene expression is a marker of melanoma, ovarian, breast, and lung cancer useful in differentiating these tissues from normal tissues and detection of these cancers in vitro or in vivo. Therapeutic modulation of this gene, expressed protein is useful in the treatment of melanoma, ovarian, breast, lung, renal and brain cancers. Moderate to low gene expression was detected in tissues with metabolic or endocrine function such as: adipose, adrenal gland, pituitary gland, and fetal skeletal muscle. Modulation of gene activity is useful in the treatment of endocrine/metabolically related diseases, such as obesity and diabetes. Significant gene expression was detected in central nervous system tissues including: amygdala, hippocampus, substantia nigra, thalamus, cerebellum, cerebral cortex, and spinal cord. Therefore, therapeutic modulation of this gene product is useful in the treatment of central nervous system disorders such as Alzheimer's disease, Parkinson's disease, epilepsy, multiple sclerosis, schizophrenia and depression.

Panel 4.1D Summary: Ag6679 Highest gene expression was seen in kidney (CT=32.6). and low gene expression was seen in resting and activated dermal fibroblasts. Therefore, therapeutic modulation of this gene, its encoded protein is useful in the treatment of kidney and skin-related disorders such as lupus, glomerulonephritis and psoriasis.

L. CG52261-01: 3277237.

Expression of gene CG52261-01 was assessed using the primer-probe sets Ag2653 and Ag044, described in Tables LA and LB. Results of the RTQ-PCR runs are shown in Tables LC, LD, LE, LF and LG.

Table LA. Probe Name Ag2653

TABLE LA

Probe Name Ag2653

| | | | | |
|---|---|---|---|---|
| | | | Start | |
| Primers | Sequences | Length | Position | SEQ ID No |

| | | | | |
|---|---|---|---|---|
| Forward | 5′-aaagatggctccggaaattat-3′ | 21 | 285 | 400 |
| Probe | TET-5′-acatgctcttttgcaacgtgaccct-3′- | 25 | 315 | 401 |
| | TAMRA | | | |
| Reverse | 5′-gattcaagaggagaggattgga-3′ | 22 | 363 | 402 |

TABLE LB

Probe Name Ag044

| | | | | |
|---|---|---|---|---|
| | | | Start | |
| Primers | Sequences | Length | Position | SEQ ID No |

| | | | | |
|---|---|---|---|---|
| Forward | 5′-tcccaaacttagttgcatagaacct-3′ | 25 | 804 | 403 |
| Probe | TET-5′-tcctgacccacgcagtccataagga-3′- | 25 | 777 | 404 |
| | TAMRA | | | |
| Reverse | 5′-tctgtgccccgtccaaa-3′ | 17 | 759 | 405 |

TABLE LC
Panel 1

| Tissue Name | A |
|---|---|
| Endothelial cells | 0.8 |
| Endothelial cells (treated) | 0.0 |
| Pancreas | 0.0 |
| Pancreatic ca. CAPAN 2 | 0.0 |
| Adrenal gland | 0.0 |
| Thyroid | 0.0 |
| Salivary gland | 0.0 |
| Pituitary gland | 0.0 |
| Brain (fetal) | 40.6 |
| Brain (whole) | 54.7 |
| Brain (amygdala) | 11.0 |
| Brain (cerebellum) | 100.0 |
| Brain (hippocampus) | 33.2 |
| Brain (substantia nigra) | 3.3 |
| Brain (thalamus) | 5.9 |
| Brain (hypothalamus) | 6.3 |
| Spinal cord | 0.4 |
| glio/astro U87-MG | 0.0 |
| glio/astro U-118-MG | 0.0 |
| astrocytoma SW1783 | 0.0 |
| neuro*; met SK-N-AS | 0.0 |
| astrocytoma SF-539 | 0.0 |
| astrocytoma SNB-75 | 0.0 |
| glioma SNB-19 | 0.0 |
| glioma U251 | 0.2 |
| glioma SF-295 | 0.0 |
| Heart | 10.3 |
| Skeletal muscle | 0.2 |
| Bone marrow | 0.0 |
| Thymus | 17.2 |
| Spleen | 0.0 |
| Lymph node | 0.0 |
| Colon (ascending) | 2.0 |
| Stomach | 0.2 |
| Small intestine | 0.5 |
| Colon ca. SW480 | 0.0 |
| Colon ca.* SW620 (SW480 met) | 0.0 |
| Colon ca. HT29 | 0.0 |
| Colon ca. HCT-116 | 0.0 |
| Colon ca. CaCo-2 | 0.0 |
| Colon ca. HCT-15 | 0.0 |
| Colon ca. HCC-2998 | 0.0 |
| Gastric ca. * (liver met) NCI-N87 | 0.0 |
| Bladder | 0.6 |
| Trachea | 0.4 |
| Kidney | 0.2 |
| Kidney (fetal) | 1.4 |
| Renal ca. 786-0 | 0.0 |
| Renal ca. A498 | 0.0 |
| Renal ca. RXF 393 | 0.0 |
| Renal ca. ACHN | 0.0 |
| Renal ca. UO-31 | 0.3 |
| Renal ca. TK-10 | 0.0 |
| Liver | 0.0 |
| Liver (fetal) | 0.0 |
| Liver ca. (hepatoblast) HepG2 | 0.0 |
| Lung | 0.0 |
| Lung (fetal) | 2.3 |
| Lung ca. (small cell) LX-1 | 0.0 |
| Lung ca. (small cell) NCI-H69 | 0.0 |
| Lung ca. (s. cell var.) SHP-77 | 0.0 |
| Lung ca. (large cell)NCI-H460 | 0.0 |
| Lung ca. (non-sm. cell) A549 | 0.0 |
| Lung ca. (non-s. cell) NCI-H23 | 0.0 |
| Lung ca. (non-s. cell) HOP-62 | 0.0 |
| Lung ca. (non-s. cl) NCI-H522 | 11.6 |
| Lung ca. (squam.) SW 900 | 1.2 |
| Lung ca. (squam.) NCI-H596 | 0.0 |
| Mammary gland | 0.9 |
| Breast ca.* (pl. ef) MCF-7 | 0.0 |
| Breast ca.* (pl. ef) MDA-MB-231 | 0.0 |
| Breast ca.* (pl. ef) T47D | 0.0 |
| Breast ca. BT-549 | 0.0 |
| Breast ca. MDA-N | 0.0 |
| Ovary | 0.0 |
| Ovarian ca. OVCAR-3 | 0.0 |
| Ovarian ca. OVCAR-4 | 0.0 |
| Ovarian ca. OVCAR-5 | 0.0 |
| Ovarian ca. OVCAR-8 | 0.8 |
| Ovarian ca. IGROV-1 | 0.0 |
| Ovarian ca. (ascites) SK-OV-3 | 0.0 |
| Uterus | 0.0 |
| Placenta | 0.0 |
| Prostate | 1.2 |
| Prostate ca.* (bone met) PC-3 | 0.0 |
| Testis | 9.3 |
| Melanoma Hs688(A).T | 0.0 |
| Melanoma* (met) Hs688(B).T | 0.0 |
| Melanoma UACC-62 | 0.0 |
| Melanoma M14 | 0.0 |
| Melanoma LOX IMVI | 0.0 |
| Melanoma* (met) SK-MEL-5 | 0.0 |
| Melanoma SK-MEL-28 | 0.0 |

Column A - Rel. Exp. (%) Ag044, Run 87361687

TABLE LD
Panel 1.3D

| Tissue Name | A |
|---|---|
| Liver adenocarcinoma | 3.9 |
| Pancreas | 1.3 |
| Pancreatic ca. CAPAN 2 | 0.0 |
| Adrenal gland | 1.3 |
| Thyroid | 0.6 |
| Salivary gland | 0.3 |
| Pituitary gland | 3.7 |
| Brain (fetal) | 100.0 |
| Brain (whole) | 40.9 |
| Brain (amygdala) | 35.6 |
| Brain (cerebellum) | 39.2 |
| Brain (hippocampus) | 38.7 |
| Brain (substantia nigra) | 8.2 |
| Brain (thalamus) | 17.8 |
| Cerebral Cortex | 19.6 |
| Spinal cord | 8.8 |
| glio/astro U87-MG | 0.0 |
| glio/astro U-118-MG | 0.0 |
| astrocytoma SW1783 | 0.0 |
| neuro*; met SK-N-AS | 0.0 |
| astrocytoma SF-539 | 0.0 |
| astrocytoma SNB-75 | 5.4 |
| glioma SNB-19 | 0.0 |
| glioma U251 | 4.1 |
| glioma SF-295 | 0.0 |
| Heart (fetal) | 0.0 |
| Heart | 14.5 |
| Skeletal muscle (fetal) | 7.3 |
| Skeletal muscle | 5.9 |
| Bone marrow | 0.0 |
| Thymus | 0.7 |
| Spleen | 0.0 |
| Lymph node | 2.5 |
| Colorectal | 3.9 |
| Stomach | 2.9 |
| Small intestine | 6.4 |
| Colon ca. SW480 | 0.0 |
| Colon ca.* SW620(SW480 met) | 0.0 |
| Colon ca. HT29 | 0.0 |
| Colon ca. HCT-116 | 0.0 |
| Colon ca. CaCo-2 | 0.0 |
| Colon ca. tissue (ODO3866) | 3.5 |
| Colon ca. HCC-2998 | 0.0 |
| Gastric ca.* (liver met) NCI-N87 | 0.0 |
| Bladder | 4.8 |
| Trachea | 0.7 |
| Kidney | 1.6 |
| Kidney (fetal) | 0.0 |
| Renal ca. 786-0 | 0.0 |
| Renal ca. A498 | 0.0 |
| Renal ca. RXF 393 | 1.3 |
| Renal ca. ACHN | 0.0 |
| Renal ca. UO-31 | 2.5 |
| Renal ca. TK-10 | 0.0 |
| Liver | 0.9 |
| Liver (fetal) | 0.0 |
| Liver ca. (hepatoblast) HepG2 | 0.0 |
| Lung | 5.3 |
| Lung (fetal) | 11.1 |
| Lung ca. (small cell) LX-1 | 0.0 |
| Lung ca. (small cell) NCI-H69 | 0.0 |
| Lung ca. (s. cell var.) SHP-77 | 0.0 |
| Lung ca. (large cell)NCI-H460 | 0.0 |
| Lung ca. (non-sm. cell) A549 | 0.0 |
| Lung ca. (non-s. cell) NCI-H23 | 2.1 |
| Lung ca. (non-s. cell) HOP-62 | 0.7 |
| Lung ca. (non-s. cl) NCI-H522 | 8.2 |
| Lung ca. (squam.) SW 900 | 0.4 |
| Lung ca. (squam.) NCI-H596 | 0.0 |
| Mammary gland | 8.4 |
| Breast ca.* (pl. ef) MCF-7 | 0.0 |
| Breast ca.* (pl. ef) MDA-MB-231 | 5.4 |
| Breast ca.* (pl. ef) T47D | 0.0 |
| Breast ca. BT-549 | 0.0 |
| Breast ca. MDA-N | 0.0 |
| Ovary | 1.4 |
| Ovarian ca. OVCAR-3 | 0.0 |
| Ovarian ca. OVCAR-4 | 0.0 |
| Ovarian ca. OVCAR-5 | 0.0 |
| Ovarian ca. OVCAR-8 | 2.6 |
| Ovarian ca. IGROV-1 | 2.3 |
| Ovarian ca.* (ascites) SK-OV-3 | 0.0 |
| Uterus | 7.5 |
| Placenta | 0.0 |
| Prostate | 1.9 |
| Prostate ca.* (bone met) PC-3 | 0.0 |
| Testis | 0.6 |
| Melanoma Hs688(A).T | 0.0 |
| Melanoma* (met) Hs688(B).T | 0.0 |
| Melanoma UACC-62 | 1.5 |
| Melanoma M14 | 0.0 |
| Melanoma LOX IMVI | 0.0 |
| Melanoma* (met) SK-MEL-5 | 0.0 |
| Adipose | 19.2 |

Column A - Rel. Exp. (%) Ag2653, Run 165720855

TABLE LE
Panel 2.2

| Tissue Name | A |
|---|---|
| Normal Colon | 6.8 |
| Colon cancer (OD06064) | 0.0 |
| Colon Margin (OD06064) | 2.7 |
| Colon cancer (OD06159) | 0.0 |
| Colon Margin (OD06159) | 10.3 |
| Colon cancer (OD06297-04) | 1.7 |
| Colon Margin (OD06297-05) | 25.0 |
| CC Gr.2 ascend colon (ODO3921) | 1.4 |
| CC Margin (ODO3921) | 4.8 |
| Colon cancer metastasis (OD06104) | 2.9 |
| Lung Margin (OD06104) | 0.0 |
| Colon mets to lung (OD04451-01) | 14.9 |
| Lung Margin (OD04451-02) | 27.2 |
| Normal Prostate | 1.2 |
| Prostate Cancer (OD04410) | 0.0 |
| Prostate Margin (OD04410) | 1.4 |
| Normal Ovary | 1.6 |
| Ovarian cancer (OD06283-03) | 0.9 |
| Ovarian Margin (OD06283-07) | 13.8 |
| Ovarian Cancer 064008 | 7.2 |
| Ovarian cancer (OD06145) | 0.0 |
| Ovarian Margin (OD06145) | 1.7 |
| Ovarian cancer (OD06455-03) | 0.0 |
| Ovarian Margin (OD06455-07) | 13.2 |
| Normal Lung | 25.9 |
| Invasive poor diff. lung adeno | 0.0 |
| (ODO4945-01) | |
| Lung Margin (ODO4945-03) | 100.0 |
| Lung Malignant Cancer (OD03126) | 7.3 |
| Lung Margin (OD03126) | 37.9 |
| Lung Cancer (OD05014A) | 3.1 |
| Lung Margin (OD05014B) | 62.9 |
| Lung cancer (OD06081) | 0.9 |
| Lung Margin (OD06081) | 54.7 |
| Lung Cancer (OD04237-01) | 0.0 |
| Lung Margin (OD04237-02) | 47.0 |
| Ocular Melanoma Metastasis | 0.0 |
| Ocular Melanoma Margin (Liver) | 1.2 |
| Melanoma Metastasis | 2.7 |
| Melanoma Margin (Lung) | 28.3 |
| Normal Kidney | 0.0 |
| Kidney Ca, Nuclear grade 2 (OD04338) | 6.2 |
| Kidney Margin (OD04338) | 1.1 |
| Kidney Ca Nuclear grade 1/2 (OD04339) | 8.9 |
| Kidney Margin (OD04339) | 1.5 |
| Kidney Ca, Clear cell type (OD04340) | 0.0 |
| Kidney Margin (OD04340) | 4.0 |
| Kidney Ca, Nuclear grade 3 (OD04348) | 1.3 |
| Kidney Margin (OD04348) | 12.7 |
| Kidney malignant cancer (OD06204B) | 0.0 |
| Kidney normal adjacent tissue | 0.0 |
| (OD06204E) | |
| Kidney Cancer (OD04450-01) | 16.7 |
| Kidney Margin (OD04450-03) | 1.7 |
| Kidney Cancer 8120613 | 0.0 |
| Kidney Margin 8120614 | 0.0 |
| Kidney Cancer 9010320 | 0.0 |
| Kidney Margin 9010321 | 0.0 |
| Kidney Cancer 8120607 | 0.0 |
| Kidney Margin 8120608 | 1.5 |
| Normal Uterus | 19.8 |
| Uterine Cancer 064011 | 2.2 |
| Normal Thyroid | 0.0 |
| Thyroid Cancer 064010 | 0.0 |
| Thyroid Cancer A302152 | 7.4 |
| Thyroid Margin A302153 | 0.5 |
| Normal Breast | 37.6 |
| Breast Cancer (OD04566) | 0.0 |
| Breast Cancer 1024 | 5.3 |
| Breast Cancer (OD04590-01) | 1.1 |
| Breast Cancer Mets (OD04590-03) | 14.7 |
| Breast Cancer Metastasis (OD04655- | 1.2 |
| 05) | |
| Breast Cancer 064006 | 7.0 |
| Breast Cancer 9100266 | 0.0 |
| Breast Margin 9100265 | 0.6 |
| Breast Cancer A209073 | 0.0 |
| Breast Margin A2090734 | 4.4 |
| Breast cancer (OD06083) | 3.3 |
| Breast cancer node metastasis | 4.1 |
| (OD06083) | |
| Normal Liver | 0.0 |
| Liver Cancer 1026 | 0.0 |
| Liver Cancer 1025 | 3.8 |
| Liver Cancer 6004-T | 0.0 |
| Liver Tissue 6004-N | 1.2 |
| Liver Cancer 6005-T | 0.0 |
| Liver Tissue 6005-N | 0.0 |
| Liver Cancer 064003 | 2.5 |
| Normal Bladder | 1.4 |
| Bladder Cancer 1023 | 3.1 |
| Bladder Cancer A302173 | 0.0 |
| Normal Stomach | 19.1 |
| Gastric Cancer 9060397 | 0.0 |
| Stomach Margin 9060396 | 0.0 |
| Gastric Cancer 9060395 | 2.7 |
| Stomach Margin 9060394 | 1.6 |
| Gastric Cancer 064005 | 3.5 |

Column A - Rel. Exp. (%) Ag2653, Run 175142263

TABLE LF
Panel 4D

| Tissue Name | A |
|---|---|
| Secondary Th1 act | 2.8 |
| Secondary Th2 act | 0.4 |
| Secondary Tr1 act | 1.7 |
| Secondary Th1 rest | 2.9 |
| Secondary Th2 rest | 0.8 |
| Secondary Tr1 rest | 0.6 |
| Primary Th1 act | 15.1 |
| Primary Th2 act | 5.3 |
| Primary Tr1 act | 9.2 |
| Primary Th1 rest | 8.0 |
| Primary Th2 rest | 1.3 |
| Primary Tr1 rest | 3.4 |
| CD45RA CD4 lymphocyte act | 1.5 |
| CD45RO CD4 lymphocyte act | 3.8 |
| CD8 lymphocyte act | 0.0 |
| Secondary CD8 lymphocyte rest | 2.4 |
| Secondary CD8 lymphocyte act | 0.0 |
| CD4 lymphocyte none | 0.0 |
| 2ry Th1/Th2/Tr1_anti-CD95 | 1.2 |
| CH11 | |
| LAK cells rest | 0.0 |
| LAK cells IL-2 | 7.1 |
| LAK cells IL-2 + IL-12 | 3.3 |
| LAK cells IL-2 + IFN gamma | 0.0 |
| LAK cells IL-2 + IL-18 | 2.1 |
| LAK cells PMA/ionomycin | 0.7 |
| NK Cells IL-2 rest | 0.6 |
| Two Way MLR 3 day | 1.1 |
| Two Way MLR 5 day | 0.0 |
| Two Way MLR 7 day | 0.0 |
| PBMC rest | 0.0 |
| PBMC PWM | 4.6 |
| PBMC PHA-L | 2.2 |
| Ramos (B cell) none | 0.0 |
| Ramos (B cell) ionomycin | 0.0 |
| B lymphocytes PWM | 14.0 |
| B lymphocytes CD40L and IL-4 | 7.9 |
| EOL-1 dbcAMP | 0.0 |
| EOL-1 dbcAMP PMA/ionomycin | 0.0 |
| Dendritic cells none | 0.4 |
| Dendritic cells LPS | 0.0 |
| Dendritic cells anti-CD40 | 0.0 |
| Monocytes rest | 0.0 |
| Monocytes LPS | 0.0 |
| Macrophages rest | 0.0 |
| Macrophages LPS | 0.0 |
| HUVEC none | 13.5 |
| HUVEC starved | 41.2 |
| HUVEC IL-1beta | 6.1 |
| HUVEC IFN gamma | 7.0 |
| HUVEC TNF alpha + IFN gamma | 13.8 |
| HUVEC TNF alpha + IL4 | 8.6 |
| HUVEC IL-11 | 6.8 |
| Lung Microvascular EC none | 7.2 |
| Lung Microvascular EC TNFalpha + IL-1beta | 6.0 |
| Microvascular Dermal EC none | 21.2 |
| Microsvasular Dermal EC TNFalpha + IL- | 11.7 |
| 1beta | |
| Bronchial epithelium TNFalpha + IL1beta | 0.3 |
| Small airway epithelium none | 2.0 |
| Small airway epithelium TNFalpha + IL-1beta | 36.3 |
| Coronery artery SMC rest | 19.3 |
| Coronery artery SMC TNFalpha + IL-1beta | 4.4 |
| Astrocytes rest | 9.3 |
| Astrocytes TNFalpha + IL-1beta | 3.5 |
| KU-812 (Basophil) rest | 0.0 |
| KU-812 (Basophil) PMA/ionomycin | 0.0 |
| CCD1106 (Keratinocytes) none | 0.3 |
| CCD1106 (Keratinocytes) TNFalpha + IL- | 0.0 |
| 1beta | |
| Liver cirrhosis | 1.8 |
| Lupus kidney | 1.9 |
| NCI-H292 none | 9.4 |
| NCI-H292 IL-4 | 8.1 |
| NCI-H292 IL-9 | 4.2 |
| NCI-H292 IL-13 | 7.1 |
| NCI-H292 IFN gamma | 4.5 |
| HPAEC none | 10.8 |
| HPAEC TNF alpha + IL-1 beta | 9.0 |
| Lung fibroblast none | 3.8 |
| Lung fibroblast TNF alpha + IL-1 beta | 0.0 |
| Lung fibroblast IL-4 | 2.2 |
| Lung fibroblast IL-9 | 2.1 |
| Lung fibroblast IL-13 | 0.7 |
| Lung fibroblast IFN gamma | 0.7 |
| Dermal fibroblast CCD1070 rest | 0.0 |
| Dermal fibroblast CCD1070 TNF alpha | 2.4 |
| Dermal fibroblast CCD1070 IL-1 beta | 0.0 |
| Dermal fibroblast IFN gamma | 12.4 |
| Dermal fibroblast IL-4 | 32.1 |
| IBD Colitis 2 | 2.2 |
| IBD Crohn's | 3.6 |
| Colon | 17.6 |
| Lung | 23.0 |
| Thymus | 6.9 |
| Kidney | 100.0 |

Column A - Rel. Exp. (%) Ag2653, Run 158944046

TABLE LG
Panel 5 Islet

| Tissue Name | A |
|---|---|
| 97457_Patient-02go_adipose | 100.0 |
| 97476_Patient-07sk_skeletal muscle | 14.7 |
| 97477_Patient-07ut_uterus | 12.2 |
| 97478_Patient-07pl_placenta | 0.0 |
| 99167_Bayer Patient 1 | 0.0 |
| 97482_Patient-08ut_uterus | 3.0 |
| 97483_Patient-08pl_placenta | 0.0 |
| 97486_Patient-09sk_skeletal muscle | 17.0 |
| 97487_Patient-09ut_uterus | 18.6 |
| 97488_Patient-09pl_placenta | 6.4 |
| 97492_Patient-10ut_uterus | 8.7 |
| 97493_Patient-10pl_placenta | 4.7 |
| 97495_Patient-11go_adipose | 57.8 |
| 97496_Patient-11sk_skeletal muscle | 38.7 |
| 97497_Patient-11ut_uterus | 19.2 |
| 97498_Patient-11pl_placenta | 0.0 |
| 97500_Patient-12go_adipose | 87.7 |
| 97501_Patient-12sk_skeletal muscle | 37.1 |
| 97502_Patient-12ut_uterus | 18.4 |
| 97503_Patient-12pl_placenta | 0.0 |
| 94721_Donor 2 U - A_Mesenchymal | 2.1 |
| Stem Cells | |
| 94722_Donor 2 U - B_Mesenchymal | 0.0 |
| Stem Cells | |
| 94723_Donor 2 U - C_Mesenchymal | 0.0 |
| Stem Cells | |
| 94709_Donor 2 AM - A_adipose | 0.0 |
| 94710_Donor 2 AM - B_adipose | 0.0 |
| 94711_Donor 2 AM - C_adipose | 0.0 |
| 94712_Donor 2 AD - A_adipose | 0.0 |
| 94713_Donor 2 AD - B_adipose | 0.0 |
| 94714_Donor 2 AD - C_adipose | 0.0 |
| 94742_Donor 3 U - A Mesenchymal Stem | 1.2 |
| Cells | |
| 94743_Donor 3 U - B Mesenchymal Stem | 0.0 |
| Cells | |
| 94730_Donor 3 AM - A_adipose | 0.0 |
| 94731_Donor 3 AM - B_adipose | 0.0 |
| 94732_Donor 3 AM - C_adipose | 0.0 |
| 94733_Donor 3 AD - A_adipose | 5.6 |
| 94734_Donor 3 AD - B_adipose | 0.0 |
| 94735_Donor 3 AD - C_adipose | 0.0 |
| 77138_Liver_HepG2untreated | 13.6 |
| 73556_Heart_Cardiac stromal cells | 12.1 |
| (primary) | |
| 81735_Small Intestine | 39.8 |
| 72409_Kidney_Proximal Convoluted | 10.1 |
| Tubule | |
| 82685_Small intestine_Duodenum | 16.8 |
| 90650_Adrenal_Adrenocortical adenoma | 0.0 |
| 72410_Kidney_HRCE | 28.7 |
| 72411_Kidney_HRE | 30.1 |
| 73139_Uterus_Uterine smooth muscle | 0.0 |
| cells | |

Column A - Rel. Exp. (%) Ag2653, Run 253239425

Panel 1 Summary: Ag044 Highest CG52261 gene expression was seen in cerebellum (CT=28) and moderate expression was detected in central nervous system tissues including: amygdala, hippocampus, substantia nigra, thalamus, cerebellum, cerebral cortex, and spinal cord. Modulation of this gene and expressed protein is useful in the treatment of central nervous system disorders such as Alzheimer's disease, Parkinson's disease, epilepsy, multiple sclerosis, schizophrenia and depression. Signigficant gene expression was also seen in fetal lung and two lung cancer cell lines indicating that gene expression level is useful as a marker to differentiate and detect the presence of lung cancer. Furthermore, therapeutic modulation of this gene and/or encoded protein is useful in the treatment of the lung cancer. Low expression of this gene was also seen in testis, thymus and heart.

Panel 1.3D Summary: Ag2653 Highest CG52261 gene expression was detected in fetal brain (CT=28.8) and moderate gene expression was seen in central nervous system tissues including: amygdala, hippocampus, substantia nigra, thalamus, cerebellum, cerebral cortex, and spinal cord. Therefore, therapeutic modulation of this gene and expressed protein is useful in the treatment of central nervous system disorders such as Alzheimer's disease, Parkinson's disease, epilepsy, multiple sclerosis, schizophrenia and depression. Significant gene expression was also seen in brain, lung, colon and breast cancer cell lines. Expression levels of this gene are useful markers to detect the presence of these cancers. Modulation of this gene and/or encoded protein is useful in the treatment of the brain, lung, colon and breast cancers. This gene is expressed at moderate levels in tissues with metabolic or endocrine function including: adipose, pituitary gland, skeletal muscle, heart, liver and the gastrointestinal tract. Therefore, therapeutic modulation of the activity of this gene is useful in the treatment of endocrine/metabolically related diseases, such as obesity and diabetes.

Panel 2.2 Summary: Ag2653 Highest CG52261 gene expression was detected in normal lung (CT=30) and significant expression of this gene was seen in normal lung, colon, breast, ovary, kidney, uterus and stomach tissues collected from tumor margins compared to low gene expression detected in breast, thyroid, kidney, lung, ovary and colon cancer samples. Therefore, modulation of this gene, encoded protein and/or use of agonist targeting the encoded protein is useful in the treatment of these lung, colon, breast, ovary, kidney, uterus and stomach cancers.

Panel 4D Summary: Ag2653 Highest expression of this gene was seen in kidney (CT=28.6) and moderate to low expression was detected in activated and resting primary Th1, Tr1, Th2 and secondary Th1 cells, activated LAK cells, activated PBMC cells, activated B lymphocytes, endothelial cells, activated small airway epithelium, coronery artery SMC cells, astrocytes, NCI-H292 cells, activated lung, dermal fibroblasts, IBD colitis and Crohn's samples. Significant expression was seen in normal colon, thymus and lung tissues. Modulation of this gene, expressed protein and/or use of antibodies, small molecule drug targeting the encoded protein alter functions associated with these cell types and relief of the symptoms of patients suffering from autoimmune and inflammatory diseases such as asthma, allergies, inflammatory bowel disease, lupus erythematosus, psoriasis, rheumatoid arthritis, and osteoarthritis.

Panel 5 Islet Summary: Ag2653 Highest expression of this gene was seen in adipose tissue from a diabetic patient (CT=32.6). Significant gene expression was seen in adipose, and skeletal muscle from either diabetic or non-diabetic but overweight patients. Low expression was seen in small intestine. Therefore, therapeutic modulation of this gene and/or expressed protein is useful in treatment of metabolic diseases such as obesity and diabetes.

M. CG52414-02: Rhomboid.

Expression of gene CG52414-02 was assessed using the primer-probe sets Ag2648, Ag2786 and Ag7066, described in Tables MA, MB and MC. Results of the RTQ-PCR runs are shown in Tables MD, ME, MF, MG and MH.

TABLE MA

Probe Name Ag2648

| | | | | |
|---|---|---|---|---|
| | | | Start | |
| Primers | Sequences | Length | Position | SEQ ID No |

| | | | | |
|---|---|---|---|---|
| Forward | 5′-ggtggatcaggtcaatcga-3′ | 19 | 1637 | 406 |
| Probe | TET-5′-caacccagaagttctcctgctggatg-3′- | 26 | 1603 | 407 |
| | TAMRA | | | |
| Reverse | 5′-gtgtgtacgagagcgtgaagta-3′ | 22 | 1581 | 408 |

TABLE MB

Probe Name Ag2786

| | | | | |
|---|---|---|---|---|
| | | | Start | |
| Primers | Sequences | Length | Position | SEQ ID No |

| | | | | |
|---|---|---|---|---|
| Forward | 5′-tggctgtacatctaccccatta-3′ | 22 | 2714 | 409 |
| Probe | TET-5′-ctggatcgagcacctcacctgctt-3′- | 24 | 2743 | 410 |
| | TAMRA | | | |
| Reverse | 5′-acctggtccagctcatacttct-3′ | 22 | 2790 | 411 |

TABLE MC

Probe Name Ag7066

| | | | | |
|---|---|---|---|---|
| | | | | SEQ ID |
| Primers | Sequences | Length | Start Position | No |

| | | | | |
|---|---|---|---|---|
| Forward | 5′-gttcagagaagcgccctg-3′ | 18 | 552 | 412 |
| Probe | TET-5′-aggcctcactgtcccagagcatc-3′- | 23 | 582 | 413 |
| | TAMRA | | | |
| Reverse | 5′-tccaaaccactgggctg-3′ | 17 | 615 | 414 |

TABLE MD
General_screening_panel_v1.7

| Tissue Name | A |
|---|---|
| Adipose | 9.2 |
| HUVEC | 16.7 |
| Melanoma* Hs688(A).T | 0.0 |
| Melanoma* Hs688(B).T | 59.5 |
| Melanoma (met) SK-MEL-5 | 31.2 |
| Testis | 1.0 |
| Prostate ca. (bone met) PC-3 | 0.3 |
| Prostate ca. DU145 | 29.1 |
| Prostate pool | 0.7 |
| Uterus pool | 1.5 |
| Ovarian ca. OVCAR-3 | 9.2 |
| Ovarian ca. (ascites) SK-OV-3 | 2.0 |
| Ovarian ca. OVCAR-4 | 100.0 |
| Ovarian ca. OVCAR-5 | 59.9 |
| Ovarian ca. IGROV-1 | 40.9 |
| Ovarian ca. OVCAR-8 | 13.8 |
| Ovary | 2.4 |
| Breast ca. MCF-7 | 11.4 |
| Breast ca. MDA-MB-231 | 71.2 |
| Breast ca. BT-549 | 3.1 |
| Breast ca. T47D | 15.7 |
| Breast pool | 2.4 |
| Trachea | 7.3 |
| Lung | 11.1 |
| Fetal Lung | 7.1 |
| Lung ca. NCI-N417 | 4.8 |
| Lung ca. LX-1 | 34.9 |
| Lung ca. NCI-H146 | 1.9 |
| Lung ca. SHP-77 | 0.9 |
| Lung ca. NCI-H23 | 59.5 |
| Lung ca. NCI-H460 | 33.2 |
| Lung ca. HOP-62 | 42.3 |
| Lung ca. NCI-H522 | 28.7 |
| Lung ca. DMS-114 | 13.1 |
| Liver | 2.2 |
| Fetal Liver | 2.3 |
| Kidney pool | 6.7 |
| Fetal Kidney | 2.9 |
| Renal ca. 786-0 | 13.9 |
| Renal ca. A498 | 63.7 |
| Renal ca. ACHN | 37.9 |
| Renal ca. UO-31 | 50.0 |
| Renal ca. TK-10 | 54.0 |
| Bladder | 3.0 |
| Gastric ca. (liver met.) NCI-N87 | 0.7 |
| Stomach | 0.3 |
| Colon ca. SW-948 | 5.8 |
| Colon ca. SW480 | 0.2 |
| Colon ca. (SW480 met) SW620 | 47.0 |
| Colon ca. HT29 | 23.0 |
| Colon ca. HCT-116 | 13.0 |
| Colon cancer tissue | 2.6 |
| Colon ca. SW1116 | 13.8 |
| Colon ca. Colo-205 | 21.3 |
| Colon ca. SW-48 | 29.9 |
| Colon | 2.5 |
| Small Intestine | 0.4 |
| Fetal Heart | 2.5 |
| Heart | 1.9 |
| Lymph Node pool 1 | 1.0 |
| Lymph Node pool 2 | 34.9 |
| Fetal Skeletal Muscle | 1.0 |
| Skeletal Muscle pool | 1.2 |
| Skeletal Muscle | 2.3 |
| Spleen | 9.9 |
| Thymus | 2.5 |
| CNS cancer (glio/astro) SF-268 | 5.1 |
| CNS cancer (glio/astro) T98G | 9.9 |
| CNS cancer (neuro; met) SK-N-AS | 3.3 |
| CNS cancer (astro) SF-539 | 10.5 |
| CNS cancer (astro) SNB-75 | 6.4 |
| CNS cancer (glio) SNB-19 | 9.3 |
| CNS cancer (glio) SF-295 | 29.1 |
| Brain (Amygdala) | 4.2 |
| Brain (Cerebellum) | 2.6 |
| Brain (Fetal) | 2.0 |
| Brain (Hippocampus) | 5.0 |
| Cerebral Cortex pool | 3.1 |
| Brain (Substantia nigra) | 2.0 |
| Brain (Thalamus) | 3.8 |
| Brain (Whole) | 5.7 |
| Spinal Cord | 5.0 |
| Adrenal Gland | 6.6 |
| Pituitary Gland | 2.0 |
| Salivary Gland | 1.8 |
| Thyroid | 3.9 |
| Pancreatic ca. PANC-1 | 40.9 |
| Pancreas pool | 3.3 |

Column A - Rel. Exp. (%) Ag7066, Run 318350037

TABLE ME
Oncology_cell_line_screening_panel_v3.2

| Tissue Name | A |
|---|---|
| 94905_Daoy_Medulloblastoma/Cere- | 5.8 |
| bellum_sscDNA | |
| 94906_TE671_Medulloblastom/Cere- | 14.5 |
| bellum_sscDNA | |
| 94907_D283 | 21.2 |
| Med_Medulloblastoma/Cere- | |
| bellum_sscDNA | |
| 94908_PFSK-1_Primitive | 3.8 |
| Neuroectodermal/Cere- | |
| bellum_sscDNA | |
| 94909_XF-498_CNS_sscDNA | 2.3 |
| 94910_SNB- | 0.0 |
| 78_CNS/glioma_sscDNA | |
| 94911_SF- | 9.4 |
| 268_CNS/glioblastoma_sscDNA | |
| 94912_T98G_Glioblastoma_sscDNA | 29.7 |
| 96776_SK-N-SH_Neuroblastoma | 23.7 |
| (metastasis)_sscDNA | |
| 94913_SF- | 45.4 |
| 295_CNS/glioblastoma_sscDNA | |
| 132565_NT2 pool_sscDNA | 12.0 |
| 94914_Cerebellum_sscDNA | 5.3 |
| 96777_Cerebellum_sscDNA | 2.5 |
| 94916_NCI-H292_Mucoepidermoid | 47.3 |
| lung carcinoma_sscDNA | |
| 94917_DMS-114_Small cell lung | 14.6 |
| cancer_sscDNA | |
| 94918_DMS-79_Small cell lung | 42.9 |
| cancer/neuroendocrine_sscDNA | |
| 94919_NCI-H146_Small cell lung | 2.9 |
| cancer/neuroendocrine_sscDNA | |
| 94920_NCI-H526_Small cell lung | 11.1 |
| cancer/neuroendocrine_sscDNA | |
| 94921_NCI-N417_Small cell lung | 5.1 |
| cancer/neuroendocrine_sscDNA | |
| 94923_NCI-H82_Small cell lung | 17.3 |
| cancer/neuroendocrine_sscDNA | |
| 94924_NCI-H157_Squamous cell lung | 34.2 |
| cancer (metastasis)_sscDNA | |
| 94925_NCI-H1155_Large cell lung | 14.6 |
| cancer/neuroendocrine_sscDNA | |
| 94926_NCI-H1299_Large cell lung | 34.6 |
| cancer/neuroendocrine_sscDNA | |
| 94927_NCI-H727_Lung | 19.6 |
| carcinoid_sscDNA | |
| 94928_NCI-UMC-11_Lung | 12.2 |
| carcinoid_sscDNA | |
| 94929_LX-1_Small cell lung | 72.7 |
| cancer_sscDNA | |
| 94930_Colo-205_Colon | 42.9 |
| cancer_sscDNA | |
| 94931_KM12_Colon cancer_sscDNA | 23.7 |
| 94932_KM20L2_Colon | 11.7 |
| cancer_sscDNA | |
| 94933_NCI-H716_Colon | 23.7 |
| cancer_sscDNA | |
| 94935_SW-48_Colon | 50.3 |
| adenocarcinoma_sscDNA | |
| 94936_SW1116_Colon | 14.9 |
| adenocarcinoma_sscDNA | |
| 94937_LS 174T_Colon | 54.0 |
| adenocarcinoma_sscDNA | |
| 94938_SW-948_Colon | 2.3 |
| adenocarcinoma_sscDNA | |
| 94939_SW-480_Colon | 29.3 |
| adenocarcinoma_sscDNA | |
| 94940_NCI-SNU-5_Gastric | 30.4 |
| carcinoma_sscDNA | |
| 112197_KATO III_Stomach_sscDNA | 19.6 |
| 94943_NCI-SNU-16_Gastric | 9.4 |
| carcinoma_sscDNA | |
| 94944_NCI-SNU-1_Gastric | 32.1 |
| carcinoma_sscDNA | |
| 94946_RF-1_Gastric | 16.7 |
| adenocarcinoma_sscDNA | |
| 94947_RF-48_Gastric | 12.2 |
| adenocarcinoma_sscDNA | |
| 96778_MKN-45_Gastric | 34.4 |
| carcinoma_sscDNA | |
| 94949_NCI-N87_Gastric | 43.5 |
| carcinoma_sscDNA | |
| 94951_OVCAR-5_Ovarian | 25.9 |
| carcinoma_sscDNA | |
| 94952_RL95-2_Uterine | 13.9 |
| carcinoma_sscDNA | |
| 94953_HelaS3_Cervical | 14.3 |
| adenocarcinoma_sscDNA | |
| 94954_Ca Ski_Cervical | 20.4 |
| epidermoid carcinoma | |
| (metastasis)_sscDNA | |
| 94955_ES-2_Ovarian clear cell | 11.3 |
| carcinoma_sscDNA | |
| 94957_Ramos/6 h stim_{—} | 13.8 |
| Stimulated with | |
| PMA/ionomycin 6 h_sscDNA | |
| 94958_Ramos/14 h stim_{—} | 24.3 |
| Stimulated with | |
| PMA/ionomycin 14 h_sscDNA | |
| 94962_MEG-01_Chronic | 12.2 |
| myelogenous leukemia | |
| (megokaryoblast)_sscDNA | |
| 94963_Raji_Burkitt's | 14.9 |
| lymphoma_sscDNA | |
| 94964_Daudi_Burkitt's | 36.9 |
| lymphoma_sscDNA | |
| 94965_U266_B-cell | 49.3 |
| plasmacytoma/myeloma_sscDNA | |
| 94968_CA46_Burkitt's | 20.6 |
| lymphoma_sscDNA | |
| 94970_RL_non-Hodgkin's B- | 18.0 |
| cell lymphoma_sscDNA | |
| 94972_JM1_pre-B-cell | 45.7 |
| lymphoma/leukemia_sscDNA | |
| 94973_Jurkat_T cell | 8.0 |
| leukemia_sscDNA | |
| 94974_TF- | 7.5 |
| 1_Erythroleukemia_sscDNA | |
| 94975_HUT 78_T-cell | 32.3 |
| lymphoma_sscDNA | |
| 94977_U937_Histiocytic | 19.5 |
| lymphoma_sscDNA | |
| 94980_KU-812_Myelogenous | 7.0 |
| leukemia_sscDNA | |
| 94981_769-P_Clear cell renal | 37.9 |
| carcinoma_sscDNA | |
| 94983_Caki-2_Clear cell renal | 58.6 |
| carcinoma_sscDNA | |
| 94984_SW 839_Clear cell | 97.9 |
| renal carcinoma_sscDNA | |
| 94986_G401_Wilms' | 12.3 |
| tumor_sscDNA | |
| 126768_293 cells_sscDNA | 19.5 |
| 94987_Hs766T_Pancreatic | 17.1 |
| carcinoma (LN | |
| metastasis)_sscDNA | |
| 94988_CAPAN-1_Pancreatic | 60.3 |
| adenocarcinoma (liver | |
| metastasis)_sscDNA | |
| 94989_SU86.86_Pancreatic | 100.0 |
| carcinoma (liver | |
| metastasis)_sscDNA | |
| 94990_BxPC-3_Pancreatic | 21.9 |
| adenocarcinoma_sscDNA | |
| 94991_HPAC_Pancreatic | 39.5 |
| adenocarcinoma_sscDNA | |
| 94992_MIA PaCa-2_Pancreatic | 11.6 |
| carcinoma_sscDNA | |
| 94993_CFPAC-1_Pancreatic | 65.1 |
| ductal | |
| adenocarcinoma_sscDNA | |
| 94994_PANC-1_Pancreatic | 87.7 |
| epithelioid ductal | |
| carcinoma_sscDNA | |
| 94996_T24_Bladder carcinma | 18.6 |
| (transitional cell)_sscDNA | |
| 94997_5637_Bladder | 74.2 |
| carcinoma_sscDNA | |
| 94998_HT-1197_Bladder | 21.0 |
| carcinoma_sscDNA | |
| 94999_UM-UC-3_Bladder | 4.9 |
| carcinma (transitional | |
| cell)_sscDNA | |
| 95000_A204_Rhabdomyosar- | 26.8 |
| coma_sscDNA | |
| 95001_HT- | 55.1 |
| 1080_Fibrosarcoma_sscDNA | |
| 95002_MG-63_Osteosarcoma | 17.6 |
| (bone)_sscDNA | |
| 95003_SK-LMS- | 24.3 |
| 1_Leiomyosarcoma | |
| (vulva)_sscDNA | |
| 95004_SJRH30_Rhabdomyosar- | 6.7 |
| coma (met to bone | |
| marrow)_sscDNA | |
| 95005_A431_Epidermoid | 24.7 |
| carcinoma_sscDNA | |
| 95007_WM266- | 11.7 |
| 4_Melanoma_sscDNA | |
| 112195_DU | 50.0 |
| 145_Prostate_sscDNA | |
| 95012_MDA-MB-468_Breast | 22.2 |
| adenocarcinoma_sscDNA | |
| 112196_SSC- | 20.7 |
| 4_Tongue_sscDNA | |
| 112194_SSC- | 84.7 |
| 9_Tongue_sscDNA | |
| 112191_SSC- | 83.5 |
| 15_Tongue_sscDNA | |
| 95017_CAL 27_Squamous cell | 50.3 |
| carcinoma of tongue_sscDNA | |

Column A - Rel. Exp. (%) Ag2648, Run 268695314

TABLE MF
Panel 1.3D

| Tissue Name | A | B |
|---|---|---|
| Liver adenocarcinoma | 20.9 | 16.5 |
| Pancreas | 10.3 | 13.0 |
| Pancreatic ca. CAPAN 2 | 19.1 | 13.0 |
| Adrenal gland | 14.5 | 18.2 |
| Thyroid | 15.2 | 13.8 |
| Salivary gland | 6.2 | 7.9 |
| Pituitary gland | 3.1 | 4.4 |
| Brain (fetal) | 2.9 | 6.6 |
| Brain (whole) | 4.7 | 18.6 |
| Brain (amygdala) | 17.3 | 32.1 |
| Brain (cerebellum) | 2.1 | 9.3 |
| Brain (hippocampus) | 50.3 | 29.5 |
| Brain (substantia nigra) | 5.4 | 34.6 |
| Brain (thalamus) | 12.2 | 29.9 |
| Cerebral Cortex | 1.9 | 5.0 |
| Spinal cord | 11.6 | 50.0 |
| glio/astro U87-MG | 20.2 | 10.2 |
| glio/astro U-118-MG | 7.1 | 6.3 |
| astrocytoma SW1783 | 19.6 | 22.7 |
| neuro*; met SK-N-AS | 33.2 | 16.7 |
| astrocytoma SF-539 | 2.6 | 7.0 |
| astrocytoma SNB-75 | 10.9 | 15.0 |
| glioma SNB-19 | 0.5 | 6.6 |
| glioma U251 | 2.5 | 17.1 |
| glioma SF-295 | 89.5 | 29.3 |
| Heart (fetal) | 10.3 | 3.4 |
| Heart | 2.6 | 6.5 |
| Skeletal muscle (fetal) | 78.5 | 8.2 |
| Skeletal muscle | 2.0 | 13.3 |
| Bone marrow | 16.6 | 20.9 |
| Thymus | 14.1 | 12.2 |
| Spleen | 55.5 | 52.9 |
| Lymph node | 19.8 | 88.3 |
| Colorectal | 10.4 | 4.9 |
| Stomach | 12.6 | 24.7 |
| Small intestine | 24.8 | 37.9 |
| Colon ca. SW480 | 31.9 | 15.7 |
| Colon ca.* SW620(SW480 met) | 37.9 | 10.2 |
| Colon ca. HT29 | 14.0 | 0.9 |
| Colon ca. HCT-116 | 17.1 | 11.2 |
| Colon ca. CaCo-2 | 14.6 | 10.4 |
| Colon ca. tissue(ODO3866) | 31.0 | 18.3 |
| Colon ca. HCC-2998 | 21.5 | 11.0 |
| Gastric ca.* (liver met) NCI-N87 | 44.4 | 46.7 |
| Bladder | 12.7 | 8.1 |
| Trachea | 42.0 | 16.7 |
| Kidney | 3.7 | 7.3 |
| Kidney (fetal) | 17.1 | 17.7 |
| Renal ca. 786-0 | 11.3 | 19.3 |
| Renal ca. A498 | 100.0 | 100.0 |
| Renal ca. RXF 393 | 18.7 | 73.2 |
| Renal ca. ACHN | 17.7 | 7.5 |
| Renal ca. UO-31 | 63.3 | 34.9 |
| Renal ca. TK-10 | 49.3 | 24.7 |
| Liver | 2.4 | 4.5 |
| Liver (fetal) | 7.9 | 10.7 |
| Liver ca. (hepatoblast) HepG2 | 35.4 | 20.9 |
| Lung | 33.2 | 40.3 |
| Lung (fetal) | 10.2 | 9.1 |
| Lung ca. (small cell) LX-1 | 46.0 | 42.3 |
| Lung ca. (small cell) NCI-H69 | 6.6 | 0.1 |
| Lung ca. (s. cell var.) SHP-77 | 3.0 | 2.0 |
| Lung ca. (large cell)NCI-H460 | 2.8 | 11.7 |
| Lung ca. (non-sm. cell) A549 | 11.7 | 7.1 |
| Lung ca. (non-s. cell) NCI-H23 | 10.4 | 11.5 |
| Lung ca. (non-s. cell) HOP-62 | 47.0 | 53.2 |
| Lung ca. (non-s. cl) NCI-H522 | 20.6 | 2.4 |
| Lung ca. (squam.) SW 900 | 12.0 | 9.0 |
| Lung ca. (squam.) NCI-H596 | 0.2 | 1.0 |
| Mammary gland | 9.5 | 23.5 |
| Breast ca.* (pl. ef) MCF-7 | 11.0 | 8.1 |
| Breast ca.* (pl. ef) MDA-MB-231 | 60.7 | 43.5 |
| Breast ca.* (pl. ef) T47D | 7.8 | 11.2 |
| Breast ca. BT-549 | 9.4 | 6.4 |
| Breast ca. MDA-N | 5.0 | 3.3 |
| Ovary | 21.9 | 1.7 |
| Ovarian ca. OVCAR-3 | 11.5 | 12.8 |
| Ovarian ca. OVCAR-4 | 6.9 | 15.5 |
| Ovarian ca. OVCAR-5 | 69.7 | 44.8 |
| Ovarian ca. OVCAR-8 | 13.3 | 2.5 |
| Ovarian ca. IGROV-1 | 5.4 | 1.7 |
| Ovarian ca.* (ascites) SK-OV-3 | 2.8 | 6.7 |
| Uterus | 2.3 | 23.7 |
| Placenta | 24.0 | 19.2 |
| Prostate | 4.2 | 11.8 |
| Prostate ca.* (bone met)PC-3 | 21.8 | 13.2 |
| Testis | 13.2 | 14.9 |
| Melanoma Hs688(A).T | 1.2 | 2.2 |
| Melanoma* (met) Hs688(B).T | 0.7 | 3.9 |
| Melanoma UACC-62 | 3.0 | 10.8 |
| Melanoma M14 | 11.1 | 47.6 |
| Melanoma LOX IMVI | 7.7 | 2.6 |
| Melanoma* (met) SK-MEL-5 | 12.3 | 5.8 |
| Adipose | 11.2 | 10.3 |

Column A - Rel. Exp. (%) Ag2648, Run 156606391
Column B - Rel. Exp. (%) Ag2786, Run 165527181

TABLE MG
Panel 2D

| Tissue Name | A | B |
|---|---|---|
| Normal Colon | 21.9 | 25.7 |
| CC Well to Mod Diff (ODO3866) | 26.4 | 30.6 |
| CC Margin (ODO3866) | 7.6 | 6.6 |
| CC Gr.2 rectosigmoid (ODO3868) | 11.7 | 10.1 |
| CC Margin (ODO3868) | 2.3 | 1.6 |
| CC Mod Diff (ODO3920) | 23.2 | 26.1 |
| CC Margin (ODO3920) | 11.3 | 7.1 |
| CC Gr.2 ascend colon (ODO3921) | 54.7 | 62.4 |
| CC Margin (ODO3921) | 10.7 | 8.7 |
| CC from Partial Hepatectomy | 58.2 | 44.1 |
| (ODO4309) Mets | | |
| Liver Margin (ODO4309) | 11.3 | 9.9 |
| Colon mets to lung (OD04451-01) | 49.7 | 41.2 |
| Lung Margin (OD04451-02) | 20.9 | 8.3 |
| Normal Prostate 6546-1 | 8.3 | 37.4 |
| Prostate Cancer (OD04410) | 18.3 | 16.4 |
| Prostate Margin (OD04410) | 15.3 | 11.3 |
| Prostate Cancer (OD04720-01) | 11.0 | 11.6 |
| Prostate Margin (OD04720-02) | 19.6 | 21.2 |
| Normal Lung 061010 | 42.3 | 39.2 |
| Lung Met to Muscle (ODO4286) | 33.9 | 37.1 |
| Muscle Margin (ODO4286) | 17.0 | 17.4 |
| Lung Malignant Cancer (OD03126) | 45.4 | 47.0 |
| Lung Margin (OD03126) | 44.1 | 29.9 |
| Lung Cancer (OD04404) | 52.5 | 33.9 |
| Lung Margin (OD04404) | 24.1 | 17.7 |
| Lung Cancer (OD04565) | 42.3 | 31.4 |
| Lung Margin (OD04565) | 28.3 | 14.0 |
| Lung Cancer (OD04237-01) | 36.3 | 35.1 |
| Lung Margin (OD04237-02) | 25.5 | 31.6 |
| Ocular Mel Met to Liver (ODO4310) | 22.1 | 25.0 |
| Liver Margin (ODO4310) | 10.3 | 7.1 |
| Melanoma Mets to Lung (OD04321) | 21.3 | 18.0 |
| Lung Margin (OD04321) | 39.2 | 34.2 |
| Normal Kidney | 17.1 | 16.8 |
| Kidney Ca, Nuclear grade 2 | 85.9 | 77.4 |
| (OD04338) | | |
| Kidney Margin (OD04338) | 26.4 | 18.6 |
| Kidney Ca Nuclear grade 1/2 | 56.6 | 46.7 |
| (OD04339) | | |
| Kidney Margin (OD04339) | 8.8 | 10.1 |
| Kidney Ca, Clear cell type | 97.9 | 100.0 |
| (OD04340) | | |
| Kidney Margin (OD04340) | 31.2 | 29.9 |
| Kidney Ca, Nuclear grade 3 | 47.6 | 40.9 |
| (OD04348) | | |
| Kidney Margin (OD04348) | 21.6 | 25.2 |
| Kidney Cancer (OD04622-01) | 60.3 | 42.6 |
| Kidney Margin (OD04622-03) | 5.7 | 4.9 |
| Kidney Cancer (OD04450-01) | 27.9 | 32.3 |
| Kidney Margin (OD04450-03) | 6.8 | 6.1 |
| Kidney Cancer 8120607 | 37.6 | 29.3 |
| Kidney Margin 8120608 | 10.4 | 7.1 |
| Kidney Cancer 8120613 | 29.9 | 33.7 |
| Kidney Margin 8120614 | 0.0 | 4.9 |
| Kidney Cancer 9010320 | 77.9 | 47.6 |
| Kidney Margin 9010321 | 27.0 | 26.1 |
| Normal Uterus | 2.8 | 2.0 |
| Uterus Cancer 064011 | 12.9 | 15.2 |
| Normal Thyroid | 15.2 | 9.9 |
| Thyroid Cancer 064010 | 13.2 | 10.4 |
| Thyroid Cancer A302152 | 19.2 | 16.0 |
| Thyroid Margin A302153 | 17.2 | 16.4 |
| Normal Breast | 19.2 | 18.8 |
| Breast Cancer (OD04566) | 37.6 | 25.2 |
| Breast Cancer (OD04590-01) | 31.0 | 34.4 |
| Breast Cancer Mets (OD04590- | 56.3 | 57.0 |
| 03) | | |
| Breast Cancer Metastasis | 28.3 | 25.7 |
| (OD04655-05) | | |
| Breast Cancer 064006 | 23.7 | 18.9 |
| Breast Cancer 1024 | 16.6 | 17.9 |
| Breast Cancer 9100266 | 33.7 | 37.1 |
| Breast Margin 9100265 | 9.9 | 13.0 |
| Breast Cancer A209073 | 42.9 | 35.4 |
| Breast Margin A209073 | 16.8 | 16.8 |
| Normal Liver | 4.5 | 7.0 |
| Liver Cancer 064003 | 6.5 | 4.8 |
| Liver Cancer 1025 | 9.3 | 4.5 |
| Liver Cancer 1026 | 25.0 | 19.6 |
| Liver Cancer 6004-T | 10.4 | 8.0 |
| Liver Tissue 6004-N | 28.1 | 15.5 |
| Liver Cancer 6005-T | 18.3 | 16.2 |
| Liver Tissue 6005-N | 6.0 | 7.0 |
| Normal Bladder | 51.4 | 66.9 |
| Bladder Cancer 1023 | 31.0 | 17.4 |
| Bladder Cancer A302173 | 27.4 | 16.2 |
| Bladder Cancer (OD04718-01) | 100.0 | 94.6 |
| Bladder Normal Adjacent | 24.0 | 22.1 |
| OD04718-03) | | |
| Normal Ovary | 8.8 | 8.3 |
| Ovarian Cancer 064008 | 74.7 | 76.8 |
| Ovarian Cancer (OD04768-07) | 82.4 | 72.2 |
| Ovary Margin (OD04768-08) | 19.9 | 12.9 |
| Normal Stomach | 11.8 | 9.5 |
| Gastric Cancer 9060358 | 8.2 | 1.8 |
| Stomach Margin 9060359 | 18.4 | 16.6 |
| Gastric Cancer 9060395 | 21.6 | 18.6 |
| Stomach Margin 9060394 | 23.5 | 17.7 |
| Gastric Cancer 9060397 | 62.0 | 63.7 |
| Stomach Margin 9060396 | 10.2 | 9.9 |
| Gastric Cancer 064005 | 24.1 | 26.2 |

Column A - Rel. Exp. (%) Ag2648, Run 156606695
Column B - Rel. Exp. (%) Ag2786, Run 162570060

TABLE MH
Panel 4D

| Tissue Name | A | B |
|---|---|---|
| Secondary Th1 act | 18.4 | 11.2 |
| Secondary Th2 act | 24.8 | 17.6 |
| Secondary Tr1 act | 20.6 | 10.4 |
| Secondary Th1 rest | 9.6 | 8.6 |
| Secondary Th2 rest | 8.2 | 7.2 |
| Secondary Tr1 rest | 9.3 | 6.7 |
| Primary Th1 act | 12.1 | 9.8 |
| Primary Th2 act | 11.0 | 5.3 |
| Primary Tr1 act | 15.2 | 5.7 |
| Primary Th1 rest | 28.3 | 15.1 |
| Primary Th2 rest | 13.6 | 9.3 |
| Primary Tr1 rest | 10.4 | 4.5 |
| CD45RA CD4 lymphocyte act | 22.8 | 9.9 |
| CD45RO CD4 lymphocyte act | 16.5 | 7.2 |
| CD8 lymphocyte act | 8.8 | 12.1 |
| Secondary CD8 lymphocyte | 15.7 | 17.4 |
| rest | | |
| Secondary CD8 lymphocyte | 8.5 | 5.1 |
| act | | |
| CD4 lymphocyte none | 7.1 | 5.6 |
| 2ry Th1/Th2/Tr1_anti-CD95 | 9.0 | 8.2 |
| CH11 | | |
| LAK cells rest | 83.5 | 58.6 |
| LAK cells IL-2 | 19.3 | 11.6 |
| LAK cells IL-2 + IL-12 | 16.0 | 15.8 |
| LAK cells IL-2 + IFN gamma | 28.5 | 17.3 |
| LAK cells IL-2 + IL-18 | 27.4 | 21.8 |
| LAK cells PMA/ionomycin | 84.1 | 60.3 |
| NK Cells IL-2 rest | 31.9 | 26.6 |
| Two Way MLR 3 day | 90.1 | 73.2 |
| Two Way MLR 5 day | 33.9 | 35.6 |
| Two Way MLR 7 day | 13.5 | 12.4 |
| PBMC rest | 12.4 | 15.3 |
| PBMC PWM | 38.2 | 27.2 |
| PBMC PHA-L | 25.3 | 23.2 |
| Ramos (B cell) none | 22.7 | 24.1 |
| Ramos (B cell) ionomycin | 49.7 | 11.3 |
| B lymphocytes PWM | 22.8 | 17.0 |
| B lymphocytes CD40L and | 26.6 | 16.2 |
| IL-4 | | |
| EOL-1 dbcAMP | 2.8 | 1.6 |
| EOL-1 dbcAMP | 33.0 | 25.5 |
| PMA/ionomycin | | |
| Dendritic cells none | 48.3 | 42.3 |
| Dendritic cells LPS | 83.5 | 68.3 |
| Dendritic cells anti-CD40 | 29.3 | 29.9 |
| Monocytes rest | 42.9 | 57.0 |
| Monocytes LPS | 80.7 | 100.0 |
| Macrophages rest | 94.6 | 82.9 |
| Macrophages LPS | 100.0 | 94.0 |
| HUVEC none | 11.3 | 8.7 |
| HUVEC starved | 19.6 | 10.6 |
| HUVEC IL-1beta | 10.2 | 4.6 |
| HUVEC IFN gamma | 33.2 | 21.9 |
| HUVEC TNF alpha + IFN gamma | 76.3 | 59.0 |
| HUVEC TNF alpha + IL4 | 34.6 | 29.9 |
| HUVEC IL-11 | 7.4 | 6.3 |
| Lung Microvascular EC none | 36.1 | 40.6 |
| Lung Microvascular EC TNFalpha + | 57.0 | 57.8 |
| IL-1beta | | |
| Microvascular Dermal EC none | 34.6 | 26.2 |
| Microsvasular Dermal EC TNFalpha + | 72.7 | 75.8 |
| IL-1beta | | |
| Bronchial epithelium TNFalpha + | 7.4 | 88.3 |
| IL1beta | | |
| Small airway epithelium none | 9.9 | 11.6 |
| Small airway epithelium TNFalpha + | 73.2 | 35.6 |
| IL-1beta | | |
| Coronery artery SMC rest | 20.7 | 17.4 |
| Coronery artery SMC TNFalpha + IL- | 20.2 | 9.6 |
| 1beta | | |
| Astrocytes rest | 8.3 | 6.8 |
| Astrocytes TNFalpha + IL-1beta | 18.2 | 15.8 |
| KU-812 (Basophil) rest | 2.3 | 1.9 |
| KU-812 (Basophil) PMA/ionomycin | 3.4 | 1.9 |
| CCD1106 (Keratinocytes) none | 19.5 | 21.8 |
| CCD1106 (Keratinocytes) TNFalpha + | 12.7 | 80.7 |
| IL-1beta | | |
| Liver cirrhosis | 4.7 | 4.6 |
| Lupus kidney | 4.2 | 2.3 |
| NCI-H292 none | 20.0 | 12.1 |
| NCI-H292 IL-4 | 23.5 | 13.5 |
| NCI-H292 IL-9 | 23.3 | 18.7 |
| NCI-H292 IL-13 | 23.7 | 15.9 |
| NCI-H292 IFN gamma | 58.6 | 38.2 |
| HPAEC none | 13.8 | 9.2 |
| HPAEC TNF alpha + IL-1 beta | 89.5 | 71.7 |
| Lung fibroblast none | 3.1 | 3.2 |
| Lung fibroblast TNF alpha + IL-1 beta | 20.0 | 15.6 |
| Lung fibroblast IL-4 | 4.4 | 2.5 |
| Lung fibroblast IL-9 | 4.3 | 3.1 |
| Lung fibroblast IL-13 | 1.6 | 2.9 |
| Lung fibroblast IFN gamma | 27.9 | 20.3 |
| Dermal fibroblast CCD1070 rest | 9.6 | 7.6 |
| Dermal fibroblast CCD1070 TNF alpha | 26.6 | 17.8 |
| Dermal fibroblast CCD1070 IL-1 beta | 8.4 | 6.9 |
| Dermal fibroblast IFN gamma | 20.2 | 12.5 |
| Dermal fibroblast IL-4 | 6.0 | 1.7 |
| IBD Colitis 2 | 0.9 | 1.1 |
| IBD Crohn's | 2.4 | 0.3 |
| Colon | 13.7 | 9.8 |
| Lung | 17.9 | 10.8 |
| Thymus | 5.8 | 1.5 |
| Kidney | 11.3 | 11.0 |

Column A - Rel. Exp. (%) Ag2648, Run 156607036
Column B - Rel. Exp. (%) Ag2786, Run 162188411

General_screening_panel_v1.7 Summary: Ag7066 Highest CG52414 gene expression was seen in OVCAR-4 ovarian cancer cell line (CT=25.6) and high expression was detected in pancreatic, gastric, colon, lung, renal, breast, ovarian, prostate, melanoma and brain cancer cell lines. Expression of this gene is a useful marker to detect the presence of these cancers. Furthermore, therapeutic modulation of the expression or function of this gene is effective in the treatment of pancreatic, gastric, colon, lung, renal, breast, ovarian, prostate, melanoma and brain cancers. Among tissues with metabolic or endocrine function, this gene was expressed at moderate levels in pancreas, adipose, adrenal gland, thyroid, pituitary gland, skeletal muscle, heart, liver and the gastrointestinal tract. Therefore, therapeutic modulation of the activity of this gene is useful in the treatment of endocrine/metabolically related diseases, such as obesity and diabetes. This gene is moderately expressed in central nervous system tissues including: amygdala, hippocampus, substantia nigra, thalamus, cerebellum, cerebral cortex, and spinal cord. Therefore, therapeutic modulation of this gene and/or encoded protein is useful in the treatment of central nervous system disorders such as Alzheimer's disease, Parkinson's disease, epilepsy, multiple sclerosis, schizophrenia and depression.

Oncology_cell_line_screening_panel_v3.2 Summary: Ag2648 Highest expression of this gene was detected in SU86.86 pancreatic cancer cell line (CT=30) and moderate gene expression was seen in lung, bone marrow, epidermoid, vulva, bone, bladder, pancreatic, renal, B cells and T cells, leukemia, lymphoma, cervical, gastric, colon, lung and brain cancer cell lines.

Panel 1.3D Summary: Ag2648/Ag2786 Highest expression of this gene was detected in renal cancer A498 cell line (CTs=28-28.9 and moderate gene expression was seen in pancreatic, gastric, colon, lung, liver, renal, breast, ovarian, prostate, melanoma and brain cancer cell lines. Thus, expression of this gene is a useful marker to detect the presence of these cancers. Furthermore, therapeutic modulation of the expression or function of this gene is effective in the treatment of pancreatic, gastric, colon, lung, liver, renal, breast, ovarian, prostate, squamous cell carcinoma, melanoma and brain cancers. Among tissues with metabolic or endocrine function, moderate gene expression was detected in pancreas, adipose, adrenal gland, thyroid, pituitary gland, skeletal muscle, heart, liver and the gastrointestinal tract. Therefore, therapeutic modulation of the activity of this gene is useful in the treatment of endocrine/metabolically related diseases, such as obesity and diabetes. This gene showed moderate to low levels of expression in central nervous system tissues including: amygdala, hippocampus, substantia nigra, thalamus, cerebellum, cerebral cortex, and spinal cord. Modulation of this gene and/or encoded protein is useful in the treatment of central nervous system disorders such as Alzheimer's disease, Parkinson's disease, epilepsy, multiple sclerosis, schizophrenia and depression.

Panel 2D Summary: Ag2648/Ag2786 Highest expression of this gene was detected in bladder and kidney cancers (CTs=26.4-28) and high to moderate gene expression was detected in cancer and normal samples derived from colon, prostate, liver, lung, kidney, breast, thyroid, ovary and stomach. Expression of this gene was higher in cancer samples especially gastric, bladder, breast, kidney and colon cancer compared to adjacent normal tissues. Expression of this gene is a useful marker to differentiate cancerous from normal adjacent tissues and to detect the presence of these cancers in vitro or in vivo. This gene codes for a protease belonging to Rhomboid family known to activate growth factors ligands (Urban et al. Cell Oct. 19, 2001; 107(2):173-82). Therefore this gene likely plays a role in tumor cell proliferation and invasion, by activating growth factors like TGFalpha and EGF that mediates cell growth and invasion. Targeting CG52414-02 protein with a human monoclonal antibody to inhibit the activity of this protein has therapeutic effect on tumors, particularly colon, gastric, kidney, ovarian and bladder tumors.

Panel 4D Summary: Ag2648/Ag2786 Highest expression of this gene was detected in LPS activated macrophages and monocytes (CTs=27-28.5) and high to moderate expression levels were detected in cell types significant in the immune response. These cells include: T-cell, B-cell, endothelial cell, macrophage/monocyte, and peripheral blood mononuclear cell family, as well as epithelial and fibroblast cell types from lung and skin, and normal colon, lung, thymus and kidney tissues. Expression of this gene is stimulated in activated endothelial cells, small airway epithelium and fibroblasts. The ubiquitous pattern of expression indicates that this gene product is involved in homeostatic processes. Modulation of the gene, expressed protein and/or antibodies, small molecule drug targeting the encoded protein alters the functions of these cell types and leads to improvement of the symptoms of patients suffering from autoimmune and inflammatory diseases such as asthma, allergies, inflammatory bowel disease, lupus erythematosus, psoriasis, rheumatoid arthritis, and osteoarthritis.

N. CG52643-02: 4324229RS.

Expression of gene CG52643-02 was assessed using the primer-probe sets Ag2812, Ag2822, Ag861, Ag10, Ag010b and Ag550, described in Tables NA, NB, NC, ND, NE and NF. Results of the RTQ-PCR runs are shown in Tables NG, NH and NI.

TABLE NA

Probe Name Ag2812

| | | | | |
|---|---|---|---|---|
| | | | Start | |
| Primers | Sequences | Length | Position | SEQ ID No |

| | | | | |
|---|---|---|---|---|
| Forward | 5′-ctgtactcgctttgtggttca-3′ | 21 | 3037 | 415 |
| Probe | TET-5′-cactggtctccttgcaagtttcctag-3′- | 26 | 3059 | 416 |
| | TAMRA | | | |
| Reverse | 5′-aatcttggtagcagcgcatac-3′ | 21 | 3091 | 417 |

TABLE NB

Probe Name Ag2822

| | | | | |
|---|---|---|---|---|
| | | | Start | SEQ ID |
| Primers | Sequences | Length | Position | No |

| | | | | |
|---|---|---|---|---|
| Forward | 5′-tcttcatccaggtcctgctt-3′ | 20 | 1023 | 418 |
| Probe | TET-5′-cttcagcacatgctgagccagttcg-3′- | 25 | 998 | 419 |
| | TAMRA | | | |
| Reverse | 5′-ttcagggacttagatgcagatg-3′ | 22 | 954 | 420 |

TABLE NC

Probe Name Ag861

| | | | | |
|---|---|---|---|---|
| | | | Start | |
| Primers | Sequences | Length | Position | SEQ ID No |

| | | | | |
|---|---|---|---|---|
| Forward | 5′-gatgggaggttttatgaaaacc-3′ | 22 | 717 | 421 |
| Probe | TET-5′-actgtaagctccaccgtgctgcttg-3′- | 25 | 739 | 422 |
| | TAMRA | | | |
| Reverse | 5′-ggatgacggtgatcctcttt-3′ | 20 | 773 | 423 |

TABLE ND

Probe Name Ag10

| | | | | |
|---|---|---|---|---|
| Primers | Sequences | Length | Start Position | SEQ ID No |

| | | | | |
|---|---|---|---|---|
| Forward | 5′-gcctggctctctggatagaca-3′ | 21 | 1439 | 424 |
| Probe | TET-5′-tggcggcacattcacctgcag-3′-TAMRA | 21 | 1410 | 425 |
| Reverse | 5′-cacgagcagctgttccagac-3′ | 20 | 1383 | 426 |

TABLE NE

Probe Name Ag010b

| | | | | |
|---|---|---|---|---|
| | | | Start | |
| Primers | Sequences | Length | Position | SEQ ID No |

| | | | | |
|---|---|---|---|---|
| Forward | 5′-tgagacatccacgccgttt-3′ | 19 | 1535 | 427 |
| Probe | TET-5′-ttcccatgcccagaatcacttggct-3′ | 25 | 1507 | 428 |
| | TAMRA | | | |
| Reverse | 5′-agccagcctaagatgccatg-3′ | 20 | 1478 | 429 |

TABLE NF

Probe Name Ag550

| | | | | |
|---|---|---|---|---|
| | | | Start | SEQ ID |
| Primers | Sequences | Length | Position | No |

| | | | | |
|---|---|---|---|---|
| Forward | 5′-ccgccccagatctcatga-3′ | 18 | 4516 | 430 |
| Probe | TET-5′-acttgtatgtctcacgcaacttggtccacc-3′- | 30 | 4542 | 431 |
| | TAMRA | | | |
| Reverse | 5′-ggcgcacccctaggagttac-3′ | 20 | 4587 | 432 |

TABLE NG
Panel 1

| Tissue Name | A | B | C |
|---|---|---|---|
| Endothelial cells | 0.0 | 0.0 | 0.0 |
| Endothelial cells (treated) | 0.0 | 0.0 | 0.0 |
| Pancreas | 0.6 | 2.2 | 1.5 |
| Pancreatic ca. CAPAN 2 | 0.0 | 0.0 | 0.0 |
| Adrenal gland | 1.3 | 5.8 | 4.8 |
| Thyroid | 0.0 | 0.0 | 0.1 |
| Salivary gland | 0.1 | 0.3 | 0.3 |
| Pituitary gland | 0.0 | 0.0 | 0.0 |
| Brain (fetal) | 0.3 | 1.9 | 1.3 |
| Brain (whole) | 2.7 | 78.5 | 24.7 |
| Brain (amygdala) | 2.7 | 13.2 | 6.9 |
| Brain (cerebellum) | 3.8 | 100.0 | 65.5 |
| Brain (hippocampus) | 1.6 | 52.1 | 8.3 |
| Brain (substantia nigra) | 1.5 | 7.3 | 4.2 |
| Brain (thalamus) | 8.2 | 48.3 | 24.5 |
| Brain (hypothalamus) | 1.6 | 15.2 | 8.0 |
| Spinal cord | 0.2 | 1.3 | 0.7 |
| glio/astro U87-MG | 0.6 | 1.5 | 1.6 |
| glio/astro U-118-MG | 0.0 | 0.0 | 0.0 |
| astrocytoma SW1783 | 0.0 | 0.0 | 0.0 |
| neuro*; met SK-N-AS | 0.0 | 0.0 | 0.3 |
| astrocytoma SF-539 | 0.0 | 0.0 | 0.0 |
| astrocytoma SNB-75 | 0.0 | 0.0 | 0.0 |
| glioma SNB-19 | 0.0 | 0.0 | 0.0 |
| glioma U251 | 0.0 | 0.0 | 0.0 |
| glioma SF-295 | 0.0 | 0.0 | 0.0 |
| Heart | 0.0 | 0.0 | 0.1 |
| Skeletal muscle | 0.0 | 0.0 | 0.1 |
| Bone marrow | 0.0 | 0.0 | 0.1 |
| Thymus | 0.2 | 1.3 | 1.3 |
| Spleen | 0.0 | 0.0 | 0.0 |
| Lymph node | 0.0 | 0.0 | 0.1 |
| Colon (ascending) | 0.4 | 0.0 | 0.1 |
| Stomach | 0.1 | 0.3 | 0.4 |
| Small intestine | 0.0 | 0.2 | 0.5 |
| Colon ca. SW480 | 0.1 | 0.0 | 0.1 |
| Colon ca.* SW620 (SW480 | 1.4 | 3.3 | 3.7 |
| met) | | | |
| Colon ca. HT29 | 0.1 | 0.0 | 0.0 |
| Colon ca. HCT-116 | 0.2 | 0.0 | 0.1 |
| Colon ca. CaCo-2 | 0.2 | 0.3 | 0.3 |
| Colon ca. HCT-15 | 0.0 | 0.0 | 0.0 |
| Colon ca. HCC-2998 | 0.0 | 0.0 | 0.1 |
| Gastric ca.* (liver met) NCI- | 0.0 | 0.0 | 0.0 |
| N87 | | | |
| Bladder | 0.3 | 5.9 | 1.3 |
| Trachea | 0.1 | 0.1 | 0.5 |
| Kidney | 3.1 | 10.5 | 3.6 |
| Kidney (fetal) | 0.4 | 1.7 | 0.9 |
| Renal ca. 786-0 | 0.0 | 0.0 | 0.0 |
| Renal ca. A498 | 0.2 | 0.0 | 0.1 |
| Renal ca. RXF 393 | 0.0 | 0.0 | 0.0 |
| Renal ca. ACHN | 0.0 | 0.0 | 0.1 |
| Renal ca. UO-31 | 0.0 | 0.0 | 0.0 |
| Renal ca. TK-10 | 0.0 | 0.0 | 0.0 |
| Liver | 0.0 | 0.0 | 0.1 |
| Liver (fetal) | 0.0 | 0.0 | 0.1 |
| Liver ca. (hepatoblast) | 6.0 | 8.2 | 6.9 |
| HepG2 | | | |
| Lung | 0.1 | 2.3 | 0.3 |
| Lung (fetal) | 0.0 | 0.0 | 0.2 |
| Lung ca. (small cell) LX-1 | 2.2 | 3.9 | 3.4 |
| Lung ca. (small cell) NCI- | 4.6 | 15.6 | 10.4 |
| H69 | | | |
| Lung ca. (s. cell var.) SHP-77 | 0.9 | 0.0 | 9.2 |
| Lung ca. (large cell)NCI- | 100.0 | 0.0 | 100.0 |
| H460 | | | |
| Lung ca. (non-sm. cell) A549 | 6.9 | 46.3 | 14.8 |
| Lung ca. (non-s. cell) NCI- | 0.3 | 0.1 | 0.4 |
| H23 | | | |
| Lung ca. (non-s. cell) HOP-62 | 0.0 | 0.0 | 0.0 |
| Lung ca. (non-s. cl) NCI- | 0.0 | 0.0 | 0.0 |
| H522 | | | |
| Lung ca. (squam.) SW 900 | 0.6 | 3.3 | 2.6 |
| Lung ca. (squam.) NCI-H596 | 9.0 | 41.8 | 22.1 |
| Mammary gland | 0.3 | 1.1 | 0.4 |
| Breast ca.* (pl. ef) MCF-7 | 2.2 | 17.7 | 9.9 |
| Breast ca.* (pl. ef) MDA-MB- | 0.0 | 0.0 | 0.0 |
| 231 | | | |
| Breast ca.* (pl. ef) T47D | 0.1 | 0.0 | 0.1 |
| Breast ca. BT-549 | 0.0 | 0.0 | 0.0 |
| Breast ca. MDA-N | 0.1 | 0.0 | 0.3 |
| Ovary | 0.2 | 0.0 | 0.4 |
| Ovarian ca. OVCAR-3 | 0.0 | 0.0 | 0.0 |
| Ovarian ca. OVCAR-4 | 0.0 | 0.5 | 0.2 |
| Ovarian ca. OVCAR-5 | 4.5 | 12.4 | 9.0 |
| Ovarian ca. OVCAR-8 | 0.0 | 0.0 | 0.0 |
| Ovarian ca. IGROV-1 | 1.3 | 3.0 | 1.7 |
| Ovarian ca. (ascites) SK-OV- | 0.0 | 0.0 | 0.0 |
| 3 | | | |
| Uterus | 2.4 | 19.3 | 7.0 |
| Placenta | 0.0 | 0.0 | 0.2 |
| Prostate | 0.7 | 5.2 | 2.1 |
| Prostate ca.* (bone met) PC-3 | 0.0 | 0.0 | 0.0 |
| Testis | 1.4 | 14.1 | 11.8 |
| Melanoma Hs688(A).T | 0.0 | 0.0 | 0.3 |
| Melanoma* (met) | 0.0 | 0.0 | 0.0 |
| Hs688(B).T | | | |
| Melanoma UACC-62 | 0.1 | 0.0 | 0.1 |
| Melanoma M14 | 0.0 | 0.0 | 0.1 |
| Melanoma LOX IMVI | 0.0 | 0.0 | 0.0 |
| Melanoma* (met) SK-MEL-5 | 0.5 | 0.7 | 1.3 |
| Melanoma SK-MEL-28 | 1.6 | 0.0 | 0.2 |

Column A - Rel. Exp. (%) Ag010b, Run 97807632
Column B - Rel. Exp. (%) Ag10, Run 87352582
Column C - Rel. Exp. (%) Ag10, Run 91677212

TABLE NH
Panel 2D

| Tissue Name | A | B |
|---|---|---|
| Normal Colon | 4.6 | 3.2 |
| CC Well to Mod Diff (ODO3866) | 0.0 | 0.0 |
| CC Margin (ODO3866) | 0.4 | 0.0 |
| CC Gr.2 rectosigmoid (ODO3868) | 0.8 | 0.0 |
| CC Margin (ODO3868) | 0.8 | 0.0 |
| CC Mod Diff (ODO3920) | 0.4 | 0.0 |
| CC Margin (ODO3920) | 0.4 | 0.8 |
| CC Gr.2 ascend colon (ODO3921) | 0.0 | 0.5 |
| CC Margin (ODO3921) | 0.3 | 0.7 |
| CC from Partial Hepatectomy | 3.1 | 0.0 |
| (ODO4309) Mets | | |
| Liver Margin (ODO4309) | 0.4 | 0.0 |
| Colon mets to lung (OD04451-01) | 1.5 | 1.4 |
| Lung Margin (OD04451-02) | 2.6 | 4.6 |
| Normal Prostate 6546-1 | 4.0 | 54.0 |
| Prostate Cancer (OD04410) | 1.9 | 3.7 |
| Prostate Margin (OD04410) | 1.4 | 1.6 |
| Prostate Cancer (OD04720-01) | 13.1 | 14.4 |
| Prostate Margin (OD04720-02) | 13.1 | 10.7 |
| Normal Lung 061010 | 14.3 | 19.2 |
| Lung Met to Muscle (ODO4286) | 1.3 | 0.0 |
| Muscle Margin (ODO4286) | 27.5 | 27.0 |
| Lung Malignant Cancer (OD03126) | 50.3 | 82.4 |
| Lung Margin (OD03126) | 18.7 | 14.2 |
| Lung Cancer (OD04404) | 13.1 | 6.2 |
| Lung Margin (OD04404) | 4.3 | 4.3 |
| Lung Cancer (OD04565) | 3.2 | 2.8 |
| Lung Margin (OD04565) | 6.2 | 6.7 |
| Lung Cancer (OD04237-01) | 7.0 | 9.1 |
| Lung Margin (OD04237-02) | 3.2 | 4.0 |
| Ocular Mel Met to Liver (ODO4310) | 3.4 | 0.0 |
| Liver Margin (ODO4310) | 0.4 | 0.0 |
| Melanoma Mets to Lung (OD04321) | 8.5 | 0.0 |
| Lung Margin (OD04321) | 9.5 | 7.5 |
| Normal Kidney | 23.2 | 33.0 |
| Kidney Ca, Nuclear grade 2 | 2.0 | 1.3 |
| (OD04338) | | |
| Kidney Margin (OD04338) | 21.5 | 13.1 |
| Kidney Ca Nuclear grade 1/2 | 4.2 | 4.3 |
| (OD04339) | | |
| Kidney Margin (OD04339) | 28.9 | 15.5 |
| Kidney Ca, Clear cell type | 4.2 | 4.7 |
| (OD04340) | | |
| Kidney Margin (OD04340) | 24.7 | 16.4 |
| Kidney Ca, Nuclear grade 3 | 0.4 | 0.0 |
| (OD04348) | | |
| Kidney Margin (OD04348) | 12.6 | 13.7 |
| Kidney Cancer (OD04622-01) | 1.4 | 0.0 |
| Kidney Margin (OD04622-03) | 4.1 | 5.6 |
| Kidney Cancer (OD04450-01) | 0.0 | 0.0 |
| Kidney Margin (OD04450-03) | 15.4 | 18.4 |
| Kidney Cancer 8120607 | 0.0 | 0.0 |
| Kidney Margin 8120608 | 5.6 | 4.1 |
| Kidney Cancer 8120613 | 0.4 | 0.7 |
| Kidney Margin 8120614 | 23.7 | 16.6 |
| Kidney Cancer 9010320 | 1.5 | 1.2 |
| Kidney Margin 9010321 | 25.9 | 25.5 |
| Normal Uterus | 0.0 | 0.0 |
| Uterus Cancer 064011 | 1.4 | 1.3 |
| Normal Thyroid | 0.8 | 0.3 |
| Thyroid Cancer 064010 | 0.7 | 0.0 |
| Thyroid Cancer A302152 | 2.6 | 0.7 |
| Thyroid Margin A302153 | 0.8 | 0.8 |
| Normal Breast | 1.8 | 2.8 |
| Breast Cancer (OD04566) | 6.6 | 7.4 |
| Breast Cancer (OD04590-01) | 10.7 | 11.3 |
| Breast Cancer Mets (OD04590- | 1.3 | 3.1 |
| 03) | | |
| Breast Cancer Metastasis | 33.0 | 43.5 |
| (OD04655-05) | | |
| Breast Cancer 064006 | 9.9 | 5.8 |
| Breast Cancer 1024 | 100.0 | 100.0 |
| Breast Cancer 9100266 | 8.0 | 6.8 |
| Breast Margin 9100265 | 1.4 | 1.0 |
| Breast Cancer A209073 | 16.2 | 8.1 |
| Breast Margin A209073 | 2.9 | 1.9 |
| Normal Liver | 0.4 | 0.0 |
| Liver Cancer 064003 | 1.0 | 0.5 |
| Liver Cancer 1025 | 0.0 | 0.0 |
| Liver Cancer 1026 | 2.2 | 2.4 |
| Liver Cancer 6004-T | 1.1 | 0.0 |
| Liver Tissue 6004-N | 2.4 | 7.0 |
| Liver Cancer 6005-T | 3.3 | 2.4 |
| Liver Tissue 6005-N | 0.0 | 0.0 |
| Normal Bladder | 5.7 | 4.5 |
| Bladder Cancer 1023 | 0.0 | 0.0 |
| Bladder Cancer A302173 | 32.8 | 20.2 |
| Bladder Cancer (OD04718-01) | 1.4 | 3.0 |
| Bladder Normal Adjacent | 0.0 | 0.0 |
| (OD04718-03) | | |
| Normal Ovary | 0.9 | 0.7 |
| Ovarian Cancer 064008 | 11.7 | 9.0 |
| Ovarian Cancer (OD04768-07) | 0.6 | 0.0 |
| Ovary Margin (OD04768-08) | 0.0 | 0.0 |
| Normal Stomach | 0.5 | 1.0 |
| Gastric Cancer 9060358 | 0.0 | 0.0 |
| Stomach Margin 9060359 | 1.4 | 0.7 |
| Gastric Cancer 9060395 | 0.0 | 0.0 |
| Stomach Margin 9060394 | 0.8 | 0.0 |
| Gastric Cancer 9060397 | 0.8 | 0.0 |
| Stomach Margin 9060396 | 1.6 | 0.0 |
| Gastric Cancer 064005 | 0.0 | 0.0 |

Column A - Rel. Exp. (%) Ag2812, Run 157457938
Column B - Rel. Exp. (%) Ag2822, Run 163578435

TABLE NI
Panel 4D

| Tissue Name | A | B | C |
|---|---|---|---|
| Secondary Th1 act | 0.0 | 0.0 | 0.0 |
| Secondary Th2 act | 0.0 | 0.0 | 2.9 |
| Secondary Tr1 act | 0.7 | 0.7 | 0.0 |
| Secondary Th1 rest | 0.0 | 0.0 | 1.6 |
| Secondary Th2 rest | 0.0 | 0.8 | 1.6 |
| Secondary Tr1 rest | 0.0 | 0.0 | 0.0 |
| Primary Th1 act | 1.4 | 2.0 | 11.6 |
| Primary Th2 act | 22.1 | 16.6 | 39.5 |
| Primary Tr1 act | 2.6 | 2.5 | 5.0 |
| Primary Th1 rest | 0.0 | 1.0 | 0.0 |
| Primary Th1 rest | 1.9 | 2.6 | 4.4 |
| Primary Tr1 rest | 0.0 | 0.8 | 0.0 |
| CD45RA CD4 | 0.8 | 0.0 | 0.0 |
| lymphocyte act | | | |
| CD45RO CD4 | 0.0 | 0.0 | 0.0 |
| lymphocyte act | | | |
| CD8 lymphocyte act | 0.0 | 0.0 | 0.0 |
| Secondary CD8 | 0.0 | 0.0 | 0.0 |
| lymphocyte rest | | | |
| Secondary CD8 | 0.0 | 0.0 | 0.0 |
| lymphocyte act | | | |
| CD4 lymphocyte none | 0.0 | 0.8 | 0.0 |
| 2ry Th1/Th2/Tr1_anti- | 0.0 | 0.2 | 0.0 |
| CD95 CH11 | | | |
| LAK cells rest | 0.8 | 0.0 | 0.0 |
| LAK cells IL-2 | 0.4 | 0.9 | 3.1 |
| LAK cells IL-2 + IL-12 | 0.0 | 0.0 | 6.3 |
| LAK cells IL-2 + IFN | 0.8 | 0.6 | 3.0 |
| gamma | | | |
| LAK cells IL-2 + IL-18 | 0.8 | 0.0 | 0.0 |
| LAK cells | 0.0 | 0.0 | 0.0 |
| PMA/ionomycin | | | |
| NK Cells IL-2 rest | 0.0 | 0.0 | 0.0 |
| Two Way MLR 3 day | 0.0 | 0.0 | 0.0 |
| Two Way MLR 5 day | 0.0 | 0.6 | 0.0 |
| Two Way MLR 7 day | 0.0 | 0.0 | 0.0 |
| PBMC rest | 0.9 | 1.8 | 0.0 |
| PBMC PWM | 0.0 | 0.9 | 0.0 |
| PBMC PHA-L | 0.0 | 1.0 | 0.0 |
| Ramos (B cell) none | 25.7 | 25.5 | 0.0 |
| Ramos (B cell) ionomycin | 100.0 | 68.3 | 0.0 |
| B lymphocytes PWM | 3.5 | 2.6 | 0.0 |
| B lymphocytes CD40L | 0.0 | 1.0 | 1.4 |
| and IL-4 | | | |
| EOL-1 dbcAMP | 0.0 | 0.0 | 0.0 |
| EOL-1 dbcAMP | 0.0 | 0.0 | 0.0 |
| PMA/ionomycin | | | |
| Dendritic cells none | 5.3 | 5.4 | 14.7 |
| Dendritic cells LPS | 0.8 | 0.0 | 0.0 |
| Dendritic cells anti-CD40 | 0.0 | 0.0 | 1.8 |
| Monocytes rest | 0.0 | 0.0 | 0.0 |
| Monocytes LPS | 0.9 | 0.0 | 0.0 |
| Macrophages rest | 0.0 | 1.9 | 0.0 |
| Macrophages LPS | 0.0 | 0.0 | 0.0 |
| HUVEC none | 0.9 | 1.0 | 0.0 |
| HUVEC starved | 1.2 | 0.0 | 0.0 |
| HUVEC IL-1beta | 0.9 | 0.0 | 0.0 |
| HUVEC IFN gamma | 0.0 | 0.0 | 0.0 |
| HUVEC TNF alpha + IFN | 0.0 | 0.0 | 0.0 |
| gamma | | | |
| HUVEC TNF alpha + IL4 | 0.0 | 0.0 | 0.0 |
| HUVEC IL-11 | 0.7 | 2.3 | 0.0 |
| Lung Microvascular EC none | 1.4 | 1.0 | 0.0 |
| Lung Microvascular EC | 0.0 | 0.0 | 0.0 |
| TNFalpha + IL-1beta | | | |
| Microvascular Dermal EC none | 2.0 | 2.0 | 0.0 |
| Microsvasular Dermal EC | 0.0 | 0.0 | 0.0 |
| TNFalpha + IL-1beta | | | |
| Bronchial epithelium | 2.1 | 1.0 | 2.1 |
| TNFalpha + IL1beta | | | |
| Small airway epithelium none | 0.9 | 0.0 | 0.0 |
| Small airway epithelium | 2.5 | 3.1 | 2.2 |
| TNFalpha + IL-1beta | | | |
| Coronery artery SMC rest | 0.0 | 0.4 | 0.0 |
| Coronery artery SMC | 0.0 | 0.0 | 0.0 |
| TNFalpha + IL-1beta | | | |
| Astrocytes rest | 0.0 | 0.0 | 0.0 |
| Astrocytes TNFalpha + IL- | 0.0 | 1.8 | 0.0 |
| 1beta | | | |
| KU-812 (Basophil) rest | 0.7 | 1.6 | 0.0 |
| KU-812 (Basophil) | 0.0 | 0.0 | 0.0 |
| PMA/ionomycin | | | |
| CCD1106 (Keratinocytes) none | 1.3 | 0.0 | 1.9 |
| CCD1106 (Keratinocytes) | 0.0 | 0.0 | 0.0 |
| TNFalpha + IL-1beta | | | |
| Liver cirrhosis | 0.0 | 2.2 | 0.0 |
| Lupus kidney | 1.1 | 1.0 | 3.4 |
| NCI-H292 none | 1.5 | 2.4 | 3.2 |
| NCI-H292 IL-4 | 2.7 | 0.0 | 4.2 |
| NCI-H292 IL-9 | 2.8 | 1.7 | 1.2 |
| NCI-H292 IL-13 | 0.4 | 2.3 | 5.1 |
| NCI-H292 IFN gamma | 0.6 | 0.6 | 0.0 |
| HPAEC none | 1.4 | 0.7 | 0.0 |
| HPAEC TNF alpha + IL-1 beta | 0.0 | 0.0 | 2.0 |
| Lung fibroblast none | 0.0 | 3.7 | 0.0 |
| Lung fibroblast TNF alpha + | 0.0 | 2.0 | 0.0 |
| IL-1 beta | | | |
| Lung fibroblast IL-4 | 0.0 | 2.6 | 0.0 |
| Lung fibroblast IL-9 | 0.0 | 5.3 | 0.0 |
| Lung fibroblast IL-13 | 0.0 | 4.1 | 0.0 |
| Lung fibroblast IFN gamma | 0.0 | 6.7 | 0.0 |
| Dermal fibroblast CCD1070 | 0.0 | 0.0 | 0.0 |
| rest | | | |
| Dermal fibroblast CCD1070 | 0.0 | 0.0 | 0.0 |
| TNF alpha | | | |
| Dermal fibroblast CCD1070 IL- | 0.0 | 0.8 | 0.0 |
| 1 beta | | | |
| Dermal fibroblast IFN gamma | 0.0 | 0.0 | 0.0 |
| Dermal fibroblast IL-4 | 0.0 | 0.0 | 0.0 |
| IBD Colitis 2 | 0.0 | 4.2 | 0.0 |
| IBD Crohn's | 0.0 | 0.0 | 3.8 |
| Colon | 5.1 | 8.2 | 2.0 |
| Lung | 9.1 | 1.0 | 5.4 |
| Thymus | 95.3 | 100.0 | 100.0 |
| Kidney | 5.7 | 16.0 | 23.0 |

Column A - Rel. Exp. (%) Ag10, Run 146090888
Column B - Rel. Exp. (%) Ag2812, Run 157457426
Column C - Rel. Exp. (%) Ag2822, Run 164320847

Panel 1 Summary: Ag10/Ag010b Highest expression of this gene was seen in NCI-H460 lung cancer cell line and cerebellum (CTs=22-24). High expression of this gene was also seen in melanoma, ovarian, lung, colon and liver cancer cell lines. Expression level of this gene is useful as a marker to detect the presence of these cancers. Furthermore, therapeutic modulation of this gene and/or expressed protein is useful in the treatment of melanoma, ovarian, lung, colon and liver cancers. Among tissues with metabolic or endocrine function, this gene was expressed at moderate levels in pancreas, adrenal gland, skeletal muscle, and the gastrointestinal tract. Therefore, therapeutic modulation of the activity of this gene is useful in the treatment of endocrine/metabolically related diseases, such as obesity and diabetes. This gene was expressed at moderate to high levels in all regions of the central nervous system examined including: amygdala, hippocampus, substantia nigra, thalamus, cerebellum, cerebral cortex, and spinal cord. Therefore, therapeutic modulation of this gene and/or expressed protein is useful in the treatment of central nervous system disorders such as Alzheimer's disease, Parkinson's disease, epilepsy, multiple sclerosis, schizophrenia and depression.

Panel 2D Summary: Ag2812/Ag2822 Highest expression of this gene was seen in breast cancer sample (CTs=28-30). Moderate to low expression of this gene was also seen in normal and cancer samples from lung, ovary, bladder, breast, kidney, and prostate. Expression of this gene was higher in bladder and breast cancer samples. Therefore, expression level of this gene is useful as a marker to detect the presence of cancer, especially bladder and breast cancer. Furthermore, therapeutic modulation of this gene and/or expressed protein is useful in the treatment of lung, ovary, bladder, breast, kidney, and prostate cancers. Higher expression of this gene was seen in kidney cancer relative to the corresponding normal sample. Thus, modulation of the expression of this gene and/or encoded protein is useful in the treatment of kidney cancer.

Panel 4D Summary: Ag10/Ag2812/Ag2822 Highest expression of this gene was seen in thymus and activated Ramos B cells (CTs=30-32). Significant expression of this gene was also seen in resting Ramos B cells, activated primary Th2 cells and kidney. Therefore, therapeutic modulation of this gene, encoded protein leads to improvement of the symptoms of patients suffering from autoimmune and inflammatory diseases such as asthma, allergies, inflammatory bowel disease, lupus erythematosus, psoriasis, rheumatoid arthritis, and osteoarthritis.

O. CG53270-01 and CG53270-02:.

Expression of gene CG53270-01 and CG53270-02 was assessed using the primer-probe sets Ag1536 and Ag1589, described in Tables OA and OB. Results of the RTQ-PCR runs are shown in Tables OC, OD, OE and DF. CG53270-02 represents the full length physical clone for CG53270-01.

TABLE OA

Probe Name Ag1536

| | | | | |
|---|---|---|---|---|
| | | | Start | |
| Primers | Sequences | Length | Position | SEQ ID No |

| | | | | |
|---|---|---|---|---|
| Forward | 5′-tcttaatgatggagcagtggtt-3′ | 22 | 222 | 433 |
| Probe | TET-5′-aacatggccagaatctcaatttcccg-3′- | 26 | 195 | 434 |
| | TAMRA | | | |
| Reverse | 5′-gcagacttcttggagaaattcc-3′ | 22 | 168 | 435 |

TABLE OB

Probe Name Ag1589

| | | | | |
|---|---|---|---|---|
| | | | Start | |
| Primers | Sequences | Length | Position | SEQ ID No |

| | | | | |
|---|---|---|---|---|
| Forward | 5′-aagaagtctgccaccaagct-3′ | 20 | 936 | 436 |
| Probe | TET-5′-cacagcctgagacaaaacccgagg-3′- | 24 | 985 | 437 |
| | TAMRA | | | |
| Reverse | 5′-cctggacatttgcattgct-3′ | 19 | 1013 | 438 |

TABLE OC
AI_comprehensive panel_v1.0

| Tissue Name | A |
|---|---|
| 110967 COPD-F | 21.3 |
| 110980 COPD-F | 5.8 |
| 110968 COPD-M | 25.0 |
| 110977 COPD-M | 17.7 |
| 110989 Emphysema-F | 39.8 |
| 110992 Emphysema-F | 24.3 |
| 110993 Emphysema-F | 22.4 |
| 110994 Emphysema-F | 13.2 |
| 110995 Emphysema-F | 29.9 |
| 110996 Emphysema-F | 13.8 |
| 110997 Asthma-M | 9.0 |
| 111001 Asthma-F | 31.0 |
| 111002 Asthma-F | 100.0 |
| 111003 Atopic Asthma-F | 46.3 |
| 111004 Atopic Asthma-F | 91.4 |
| 111005 Atopic Asthma-F | 41.2 |
| 111006 Atopic Asthma-F | 9.9 |
| 111417 Allergy-M | 68.8 |
| 112347 Allergy-M | 0.1 |
| 112349 Normal Lung-F | 0.0 |
| 112357 Normal Lung-F | 46.0 |
| 112354 Normal Lung-M | 8.8 |
| 112374 Crohns-F | 30.1 |
| 112389 Match Control Crohns-F | 17.2 |
| 112375 Crohns-F | 22.7 |
| 112732 Match Control Crohns-F | 22.8 |
| 112725 Crohns-M | 4.0 |
| 112387 Match Control Crohns-M | 9.4 |
| 112378 Crohns-M | 0.8 |
| 112390 Match Control Crohns-M | 98.6 |
| 112726 Crohns-M | 47.3 |
| 112731 Match Control Crohns-M | 34.9 |
| 112380 Ulcer Col-F | 73.7 |
| 112734 Match Control Ulcer Col-F | 32.1 |
| 112384 Ulcer Col-F | 23.2 |
| 112737 Match Control Ulcer Col-F | 12.0 |
| 112386 Ulcer Col-F | 9.4 |
| 112738 Match Control Ulcer Col-F | 1.5 |
| 112381 Ulcer Col-M | 0.0 |
| 112735 Match Control Ulcer Col-M | 1.2 |
| 112382 Ulcer Col-M | 38.4 |
| 112394 Match Control Ulcer Col-M | 5.5 |
| 112383 Ulcer Col-M | 38.7 |
| 112736 Match Control Ulcer Col-M | 21.8 |
| 112423 Psoriasis-F | 22.2 |
| 112427 Match Control Psoriasis-F | 33.4 |
| 112418 Psoriasis-M | 24.1 |
| 112723 Match Control Psoriasis-M | 43.2 |
| 112419 Psoriasis-M | 18.0 |
| 112424 Match Control Psoriasis-M | 2.0 |
| 112420 Psoriasis-M | 41.2 |
| 112425 Match Control Psoriasis-M | 60.7 |
| 104689 (MF) OA Bone-Backus | 20.4 |
| 104690 (MF) Adj “Normal” Bone-Backus | 5.2 |
| 104691 (MF) OA Synovium-Backus | 4.7 |
| 104692 (BA) OA Cartilage-Backus | 2.8 |
| 104694 (BA) OA Bone-Backus | 46.3 |
| 104695 (BA) Adj “Normal” Bone-Backus | 14.1 |
| 104696 (BA) OA Synovium-Backus | 15.2 |
| 104700 (SS) OA Bone-Backus | 6.3 |
| 104701 (SS) Adj “Normal” Bone-Backus | 12.4 |
| 104702 (SS) OA Synovium-Backus | 12.0 |
| 117093 OA Cartilage Rep7 | 58.2 |
| 112672 OA Bone5 | 50.7 |
| 112673 OA Synovium5 | 17.9 |
| 112674 OA Synovial Fluid cells5 | 11.6 |
| 117100 OA Cartilage Rep14 | 2.5 |
| 112756 OA Bone9 | 2.8 |
| 112757 OA Synovium9 | 16.0 |
| 112758 OA Synovial Fluid Cells9 | 18.2 |
| 117125 RA Cartilage Rep2 | 56.3 |
| 113492 Bone2 RA | 6.8 |
| 113493 Synovium2 RA | 2.8 |
| 113494 Syn Fluid Cells RA | 3.7 |
| 113499 Cartilage4 RA | 2.1 |
| 113500 Bone4 RA | 3.1 |
| 113501 Synovium4 RA | 1.6 |
| 113502 Syn Fluid Cells4 RA | 0.1 |
| 113495 Cartilage3 RA | 1.5 |
| 113496 Bone3 RA | 2.5 |
| 113497 Synovium3 RA | 0.6 |
| 113498 Syn Fluid Cells3 RA | 0.4 |
| 117106 Normal Cartilage Rep20 | 5.5 |
| 113663 Bone3 Normal | 0.0 |
| 113664 Synovium3 Normal | 0.0 |
| 113665 Syn Fluid Cells3 Normal | 0.0 |
| 117107 Normal Cartilage Rep22 | 5.2 |
| 113667 Bone4 Normal | 19.3 |
| 113668 Synovium4 Normal | 11.3 |
| 113669 Syn Fluid Cells4 Normal | 23.5 |

Column A - Rel. Exp. (%) Ag1589, Run 278182088

TABLE OD
Panel 1.2

| Tissue Name | A |
|---|---|
| Endothelial cells | 1.9 |
| Heart (Fetal) | 0.6 |
| Pancreas | 2.0 |
| Pancreatic ca. CAPAN 2 | 0.5 |
| Adrenal Gland | 2.0 |
| Thyroid | 0.0 |
| Salivary gland | 6.7 |
| Pituitary gland | 0.1 |
| Brain (fetal) | 0.1 |
| Brain (whole) | 0.4 |
| Brain (amygdala) | 0.4 |
| Brain (cerebellum) | 0.4 |
| Brain (hippocampus) | 1.3 |
| Brain (thalamus) | 0.3 |
| Cerebral Cortex | 4.2 |
| Spinal cord | 0.2 |
| glio/astro U87-MG | 0.2 |
| glio/astro U-118-MG | 2.7 |
| astrocytoma SW1783 | 1.8 |
| neuro*; met SK-N-AS | 6.0 |
| astrocytoma SF-539 | 0.9 |
| astrocytoma SNB-75 | 0.7 |
| glioma SNB-19 | 2.7 |
| glioma U251 | 1.7 |
| glioma SF-295 | 1.7 |
| Heart | 4.0 |
| Skeletal Muscle | 0.7 |
| Bone marrow | 0.1 |
| Thymus | 0.0 |
| Spleen | 0.0 |
| Lymph node | 0.0 |
| Colorectal Tissue | 0.4 |
| Stomach | 0.4 |
| Small intestine | 0.6 |
| Colon ca. SW480 | 0.2 |
| Colon ca.* SW620 (SW480 met) | 0.0 |
| Colon ca. HT29 | 0.5 |
| Colon ca. HCT-116 | 2.0 |
| Colon ca. CaCo-2 | 0.0 |
| Colon ca. Tissue (ODO3866) | 0.8 |
| Colon ca. HCC-2998 | 6.0 |
| Gastric ca.* (liver met) NCI-N87 | 4.5 |
| Bladder | 3.9 |
| Trachea | 0.2 |
| Kidney | 1.3 |
| Kidney (fetal) | 2.3 |
| Renal ca. 786-0 | 3.1 |
| Renal ca. A498 | 7.8 |
| Renal ca. RXF 393 | 0.0 |
| Renal ca. ACHN | 1.4 |
| Renal ca. UO-31 | 6.6 |
| Renal ca. TK-10 | 3.8 |
| Liver | 0.5 |
| Liver (fetal) | 0.2 |
| Liver ca. (hepatoblast) HepG2 | 5.2 |
| Lung | 0.0 |
| Lung (fetal) | 0.0 |
| Lung ca. (small cell) LX-1 | 0.1 |
| Lung ca. (small cell) NCI-H69 | 3.5 |
| Lung ca. (s. cell var.) SHP-77 | 1.3 |
| Lung ca. (large cell)NCI-H460 | 4.5 |
| Lung ca. (non-sm. cell) A549 | 1.0 |
| Lung ca. (non-s. cell) NCI-H23 | 1.0 |
| Lung ca. (non-s. cell) HOP-62 | 4.2 |
| Lung ca. (non-s. cl) NCI-H522 | 32.3 |
| Lung ca. (squam.) SW 900 | 4.6 |
| Lung ca. (squam.) NCI-H596 | 1.9 |
| Mammary gland | 2.0 |
| Breast ca.* (pl. ef) MCF-7 | 3.7 |
| Breast ca.* (pl. ef) MDA-MB-231 | 0.1 |
| Breast ca.* (pl. ef) T47D | 8.1 |
| Breast ca. BT-549 | 0.2 |
| Breast ca. MDA-N | 0.3 |
| Ovary | 1.8 |
| Ovarian ca. OVCAR-3 | 1.6 |
| Ovarian ca. OVCAR-4 | 1.9 |
| Ovarian ca. OVCAR-5 | 9.3 |
| Ovarian ca. OVCAR-8 | 1.0 |
| Ovarian ca. IGROV-1 | 0.6 |
| Ovarian ca. (ascites) SK-OV-3 | 1.8 |
| Uterus | 0.5 |
| Placenta | 0.1 |
| Prostate | 8.7 |
| Prostate ca.* (bone met) PC-3 | 0.2 |
| Testis | 100.0 |
| Melanoma Hs688(A).T | 0.6 |
| Melanoma* (met) Hs688(B).T | 1.4 |
| Melanoma UACC-62 | 1.7 |
| Melanoma M14 | 2.1 |
| Melanoma LOX IMVI | 0.0 |
| Melanoma* (met) SK-MEL-5 | 0.1 |

Column A - Rel. Exp. (%) Ag1536, Run 142232138

TABLE OE
Panel 2D

| Tissue Name | A | B | C | D |
|---|---|---|---|---|
| Normal Colon | 5.2 | 15.2 | 17.7 | 3.8 |
| CC Well to Mod Diff (ODO3866) | 2.2 | 7.1 | 3.4 | 4.2 |
| CC Margin (ODO3866) | 3.8 | 2.8 | 2.3 | 0.0 |
| CC Gr.2 rectosigmoid (ODO3868) | 11.2 | 8.5 | 14.4 | 3.7 |
| CC Margin (ODO3868) | 2.4 | 1.8 | 0.0 | 0.0 |
| CC Mod Diff (ODO3920) | 1.1 | 2.6 | 4.7 | 0.0 |
| CC Margin (ODO3920) | 1.7 | 3.0 | 5.3 | 2.3 |
| CC Gr.2 ascend colon (ODO3921) | 0.0 | 1.8 | 3.6 | 3.7 |
| CC Margin (ODO3921) | 1.9 | 1.3 | 1.9 | 0.3 |
| CC from Partial Hepatectomy | 0.0 | 0.0 | 2.2 | 1.0 |
| (ODO4309) Mets | | | | |
| Liver Margin (ODO4309) | 0.0 | 1.2 | 3.5 | 3.8 |
| Colon mets to lung (OD04451-01) | 2.0 | 3.0 | 4.9 | 0.0 |
| Lung Margin (OD04451-02) | 2.4 | 2.0 | 1.6 | 0.0 |
| Normal Prostate 6546-1 | 71.2 | 28.7 | 17.7 | 25.5 |
| Prostate Cancer (OD04410) | 57.0 | 27.9 | 35.6 | 34.6 |
| Prostate Margin (OD04410) | 39.2 | 32.1 | 72.2 | 31.0 |
| Prostate Cancer (OD04720-01) | 54.7 | 74.2 | 85.3 | 30.4 |
| Prostate Margin (OD04720-02) | 72.7 | 85.9 | 100.0 | 100.0 |
| Normal Lung 061010 | 8.8 | 1.5 | 3.5 | 28.7 |
| Lung Met to Muscle (ODO4286) | 0.0 | 4.2 | 8.4 | 6.4 |
| Muscle Margin (ODO4286) | 8.4 | 3.4 | 3.0 | 0.0 |
| Lung Malignant Cancer (OD03126) | 0.0 | 1.6 | 3.4 | 2.2 |
| Lung Margin (OD03126) | 0.0 | 1.2 | 1.9 | 4.5 |
| Lung Cancer (OD04404) | 11.1 | 10.2 | 20.6 | 2.9 |
| Lung Margin (OD04404) | 8.7 | 10.7 | 5.6 | 4.8 |
| Lung Cancer (OD04565) | 13.9 | 14.9 | 19.2 | 25.7 |
| Lung Margin (OD04565) | 2.3 | 3.6 | 3.5 | 0.0 |
| Lung Cancer (OD04237-01) | 11.3 | 0.0 | 1.4 | 0.0 |
| Lung Margin (OD04237-02) | 1.1 | 1.0 | 1.9 | 6.6 |
| Ocular Mel Met to Liver (ODO4310) | 0.0 | 0.0 | 0.0 | 0.0 |
| Liver Margin (ODO4310) | 1.9 | 0.0 | 0.0 | 0.0 |
| Melanoma Mets to Lung (OD04321) | 1.8 | 2.4 | 1.5 | 0.3 |
| Lung Margin (OD04321) | 0.0 | 0.0 | 0.0 | 0.5 |
| Normal Kidney | 4.3 | 3.6 | 5.3 | 8.7 |
| Kidney Ca, Nuclear grade 2 (OD04338) | 7.1 | 8.8 | 4.5 | 2.3 |
| Kidney Margin (OD04338) | 0.0 | 4.3 | 1.7 | 2.7 |
| Kidney Ca Nuclear grade 1/2 (OD04339) | 10.2 | 10.5 | 10.8 | 24.1 |
| Kidney Margin (OD04339) | 0.0 | 4.7 | 0.9 | 0.0 |
| Kidney Ca, Clear cell type (OD04340) | 0.0 | 0.0 | 0.0 | 2.6 |
| Kidney Margin (OD04340) | 0.0 | 0.0 | 1.9 | 2.0 |
| Kidney Ca, Nuclear grade 3 (OD04348) | 1.9 | 1.5 | 0.0 | 0.0 |
| Kidney Margin (OD04348) | 2.3 | 2.6 | 3.5 | 1.2 |
| Kidney Cancer (OD04622-01) | 14.5 | 12.7 | 4.7 | 13.7 |
| Kidney Margin (OD04622-03) | 3.3 | 0.0 | 0.0 | 0.0 |
| Kidney Cancer (OD04450-01) | 0.0 | 0.0 | 0.0 | 0.0 |
| Kidney Margin (OD04450-03) | 4.2 | 0.0 | 3.5 | 0.0 |
| Kidney Cancer 8120607 | 0.0 | 0.0 | 0.0 | 2.9 |
| Kidney Margin 8120608 | 1.2 | 2.6 | 0.0 | 0.0 |
| Kidney Cancer 8120613 | 0.0 | 0.0 | 1.6 | 0.0 |
| Kidney Margin 8120614 | 0.0 | 6.0 | 0.0 | 0.0 |
| Kidney Cancer 9010320 | 13.7 | 11.0 | 1.7 | 8.4 |
| Kidney Margin 9010321 | 0.0 | 1.4 | 0.0 | 0.0 |
| Normal Uterus | 11.1 | 5.2 | 4.7 | 0.0 |
| Uterus Cancer 064011 | 8.5 | 8.4 | 8.7 | 3.9 |
| Normal Thyroid | 2.2 | 0.0 | 3.5 | 0.0 |
| Thyroid Cancer 064010 | 9.5 | 3.7 | 5.2 | 0.0 |
| Thyroid Cancer A302152 | 1.6 | 4.5 | 13.0 | 7.5 |
| Thyroid Margin A302153 | 0.0 | 0.0 | 0.0 | 0.0 |
| Normal Breast | 19.3 | 35.8 | 12.0 | 17.2 |
| Breast Cancer (OD04566) | 16.8 | 20.0 | 5.4 | 5.0 |
| Breast Cancer (OD04590-01) | 100.0 | 100.0 | 70.2 | 90.8 |
| Breast Cancer Mets (OD04590-03) | 71.2 | 45.1 | 65.5 | 50.0 |
| Breast Cancer Metastasis (OD04655-05) | 53.2 | 50.3 | 81.2 | 67.8 |
| Breast Cancer 064006 | 15.8 | 26.1 | 21.0 | 17.7 |
| Breast Cancer 1024 | 49.3 | 45.1 | 50.3 | 23.5 |
| Breast Cancer 9100266 | 10.9 | 10.1 | 7.3 | 6.9 |
| Breast Margin 9100265 | 9.3 | 16.6 | 16.5 | 15.2 |
| Breast Cancer A209073 | 10.8 | 12.6 | 10.1 | 16.8 |
| Breast Margin A209073 | 36.1 | 34.2 | 51.1 | 43.5 |
| Normal Liver | 0.0 | 2.2 | 1.2 | 2.5 |
| Liver Cancer 064003 | 2.7 | 2.3 | 4.0 | 0.0 |
| Liver Cancer 1025 | 4.0 | 0.0 | 3.3 | 0.0 |
| Liver Cancer 1026 | 0.0 | 0.0 | 0.0 | 0.0 |
| Liver Cancer 6004-T | 4.6 | 1.3 | 2.0 | 1.8 |
| Liver Tissue 6004-N | 2.2 | 1.5 | 1.4 | 2.2 |
| Liver Cancer 6005-T | 0.0 | 0.0 | 2.2 | 3.0 |
| Liver Tissue 6005-N | 0.0 | 0.0 | 0.0 | 0.0 |
| Normal Bladder | 3.3 | 5.4 | 7.3 | 3.7 |
| Bladder Cancer 1023 | 4.8 | 0.0 | 1.6 | 14.9 |
| Bladder Cancer A302173 | 24.1 | 17.8 | 13.5 | 12.0 |
| Bladder Cancer (OD04718-01) | 2.6 | 5.7 | 1.2 | 0.0 |
| Bladder Normal Adjacent (OD04718-03) | 4.9 | 1.2 | 3.9 | 1.3 |
| Normal Ovary | 3.1 | 2.9 | 6.6 | 8.3 |
| Ovarian Cancer 064008 | 21.5 | 12.1 | 12.7 | 5.9 |
| Ovarian Cancer (OD04768-07) | 0.0 | 1.9 | 0.0 | 6.8 |
| Ovary Margin (OD04768-08) | 0.0 | 0.0 | 2.0 | 3.1 |
| Normal Stomach | 0.0 | 2.7 | 2.6 | 4.3 |
| Gastric Cancer 9060358 | 0.0 | 0.0 | 0.0 | 2.2 |
| Stomach Margin 9060359 | 0.0 | 1.5 | 0.0 | 0.0 |
| Gastric Cancer 9060395 | 3.8 | 5.7 | 1.6 | 5.2 |
| Stomach Margin 9060394 | 0.0 | 1.8 | 2.1 | 5.5 |
| Gastric Cancer 9060397 | 0.0 | 4.1 | 4.4 | 13.9 |
| Stomach Margin 9060396 | 0.0 | 2.5 | 0.0 | 0.0 |
| Gastric Cancer 064005 | 2.4 | 4.9 | 8.9 | 17.9 |

Column A - Rel. Exp. (%) Ag1536, Run 145177050
Column B - Rel. Exp. (%) Ag1536, Run 147091203
Column C - Rel. Exp. (%) Ag1536, Run 147758040
Column D - Rel. Exp. (%) Ag1589, Run 155518625

TABLE OF
Panel 4D

| Tissue Name | A | B | C |
|---|---|---|---|
| Secondary Th1 act | 0.0 | 0.0 | 0.0 |
| Secondary Th2 act | 0.0 | 0.0 | 0.0 |
| Secondary Tr1 act | 1.5 | 1.6 | 0.0 |
| Secondary Th1 rest | 0.0 | 0.0 | 0.0 |
| Secondary Th2 rest | 0.0 | 0.0 | 0.0 |
| Secondary Tr1 rest | 0.0 | 0.0 | 0.0 |
| Primary Th1 act | 0.0 | 0.0 | 0.0 |
| Primary Th2 act | 1.6 | 0.0 | 0.0 |
| Primary Tr1 act | 0.0 | 0.0 | 0.0 |
| Primary Th1 rest | 0.0 | 0.0 | 0.0 |
| Primary Th2 rest | 0.0 | 0.0 | 0.0 |
| Primary Tr1 rest | 0.0 | 2.0 | 0.0 |
| CD45RA CD4 lymphocyte | 3.2 | 0.0 | 5.0 |
| act | | | |
| CD45RO CD4 lymphocyte | 0.0 | 0.0 | 0.0 |
| act | | | |
| CD8 lymphocyte act | 0.0 | 0.0 | 0.0 |
| Secondary CD8 | 0.0 | 0.0 | 0.0 |
| lymphocyte rest | | | |
| Secondary CD8 | 0.0 | 0.0 | 0.0 |
| lymphocyte act | | | |
| CD4 lymphocyte none | 0.0 | 0.0 | 0.0 |
| 2ry Th1/Th2/Tr1_anti- | 3.8 | 1.6 | 0.0 |
| CD95 CH11 | | | |
| LAK cells rest | 1.9 | 0.0 | 0.0 |
| LAK cells IL-2 | 0.0 | 0.0 | 0.0 |
| LAK cells IL-2 + IL-12 | 0.0 | 0.0 | 0.0 |
| LAK cells IL-2 + IFN | 1.7 | 0.0 | 0.0 |
| gamma | | | |
| LAK cells IL-2 + IL-18 | 0.0 | 0.0 | 0.0 |
| LAK cells PMA/ionomycin | 0.0 | 0.0 | 0.0 |
| NK Cells IL-2 rest | 0.0 | 0.0 | 0.0 |
| Two Way MLR 3 day | 0.0 | 0.0 | 0.0 |
| Two Way MLR 5 day | 0.0 | 0.0 | 0.0 |
| Two Way MLR 7 day | 0.0 | 0.0 | 0.0 |
| PBMC rest | 0.0 | 0.0 | 0.0 |
| PBMC PWM | 0.0 | 1.7 | 3.3 |
| PBMC PHA-L | 3.1 | 0.0 | 0.0 |
| Ramos (B cell) none | 0.0 | 0.0 | 0.0 |
| Ramos (B cell) ionomycin | 0.0 | 0.0 | 0.0 |
| B lymphocytes PWM | 0.0 | 0.0 | 0.0 |
| B lymphocytes CD40L and | 1.9 | 1.2 | 5.1 |
| IL-4 | | | |
| EOL-1 dbcAMP | 0.0 | 0.0 | 0.0 |
| EOL-1 dbcAMP | 0.0 | 0.0 | 0.0 |
| PMA/ionomycin | | | |
| Dendritic cells none | 0.0 | 0.0 | 0.0 |
| Dendritic cells LPS | 0.0 | 0.0 | 0.0 |
| Dendritic cells anti-CD40 | 0.0 | 0.0 | 0.0 |
| Monocytes rest | 0.0 | 0.0 | 0.0 |
| Monocytes LPS | 0.0 | 0.0 | 0.0 |
| Macrophages rest | 0.0 | 1.7 | 0.0 |
| Macrophages LPS | 0.0 | 0.0 | 2.0 |
| HUVEC none | 4.9 | 0.0 | 10.4 |
| HUVEC starved | 6.9 | 7.2 | 0.0 |
| HUVEC IL-1beta | 0.0 | 0.0 | 0.0 |
| HUVEC IFN gamma | 3.9 | 5.6 | 0.0 |
| HUVEC TNF alpha + IFN | 0.0 | 0.0 | 0.0 |
| gamma | | | |
| HUVEC TNF alpha + IL4 | 0.0 | 0.0 | 0.0 |
| HUVEC IL-11 | 2.0 | 2.6 | 6.5 |
| Lung Microvascular EC none | 0.0 | 8.0 | 0.0 |
| Lung Microvascular EC | 0.0 | 0.0 | 0.0 |
| TNFalpha + IL-1beta | | | |
| Microvascular Dermal EC none | 2.0 | 3.6 | 0.0 |
| Microsvasular Dermal EC | 1.5 | 0.0 | 0.0 |
| TNFalpha + IL-1beta | | | |
| Bronchial epithelium TNFalpha + | 21.3 | 21.9 | 20.2 |
| IL1beta | | | |
| Small airway epithelium none | 12.0 | 18.0 | 0.0 |
| Small airway epithelium | 100.0 | 100.0 | 52.1 |
| TNFalpha + IL-1beta | | | |
| Coronery artery SMC rest | 2.9 | 4.4 | 0.0 |
| Coronery artery SMC | 13.9 | 4.2 | 0.0 |
| TNFalpha + IL-1beta | | | |
| Astrocytes rest | 30.4 | 15.2 | 11.4 |
| Astrocytes TNFalpha + IL-1beta | 3.8 | 8.5 | 3.9 |
| KU-812 (Basophil) rest | 0.0 | 0.0 | 0.0 |
| KU-812 (Basophil) | 0.0 | 0.0 | 0.0 |
| PMA/ionomycin | | | |
| CCD1106 (Keratinocytes) none | 43.8 | 28.5 | 26.6 |
| CCD1106 (Keratinocytes) | 1.7 | 0.0 | 4.5 |
| TNFalpha + IL-1beta | | | |
| Liver cirrhosis | 14.2 | 5.7 | 37.9 |
| Lupus kidney | 0.0 | 1.5 | 0.0 |
| NCI-H292 none | 57.8 | 53.6 | 27.4 |
| NCI-H292 IL-4 | 77.9 | 63.7 | 100.0 |
| NCI-H292 IL-9 | 64.2 | 81.2 | 43.2 |
| NCI-H292 IL-13 | 52.5 | 28.5 | 56.6 |
| NCI-H292 IFN gamma | 41.8 | 22.1 | 25.0 |
| HPAEC none | 9.2 | 19.3 | 17.6 |
| HPAEC TNF alpha + IL-1 beta | 6.9 | 4.5 | 9.1 |
| Lung fibroblast none | 16.2 | 16.3 | 15.1 |
| Lung fibroblast TNF alpha + IL-1 | 8.2 | 13.5 | 3.4 |
| beta | | | |
| Lung fibroblast IL-4 | 26.4 | 31.9 | 0.0 |
| Lung fibroblast IL-9 | 6.2 | 11.9 | 14.3 |
| Lung fibroblast IL-13 | 33.7 | 23.2 | 18.3 |
| Lung fibroblast IFN gamma | 16.6 | 16.8 | 0.0 |
| Dermal fibroblast CCD1070 rest | 10.9 | 6.7 | 0.0 |
| Dermal fibroblast CCD1070 TNF | 1.8 | 9.7 | 10.0 |
| alpha | | | |
| Dermal fibroblast CCD1070 IL-1 | 5.3 | 0.8 | 0.0 |
| beta | | | |
| Dermal fibroblast IFN gamma | 2.2 | 3.3 | 4.6 |
| Dermal fibroblast IL-4 | 21.0 | 11.0 | 9.2 |
| IBD Colitis 2 | 6.1 | 1.3 | 0.0 |
| IBD Crohn's | 1.6 | 0.0 | 0.0 |
| Colon | 1.7 | 12.5 | 0.0 |
| Lung | 15.5 | 18.4 | 16.8 |
| Thymus | 1.7 | 0.9 | 11.6 |
| Kidney | 2.6 | 2.6 | 0.0 |

Column A - Rel. Exp. (%) Ag1536, Run 147091255
Column B - Rel. Exp. (%) Ag1536, Run 147758086
Column C - Rel. Exp. (%) Ag1589, Run 146791435

AI_comprehensive_panel_v1.0 Summary: Ag1589 Highest expression of this gene was seen in an asthma sample (CT=30). Moderate levels of expression of this gene were detected in samples derived from normal and orthoarthitis bone and adjacent bone, cartilage, synovium and synovial fluid samples, rheumatoid arthritis bone and cartilage, normal lung, COPD lung, emphysema, atopic asthma, asthma, Crohn's disease (normal matched control and diseased), ulcerative colitis (normal matched control and diseased), and psoriasis (normal matched control and diseased). Therefore, therapeutic modulation of this gene and/or expressed protein ameliorate symptoms/conditions associated with autoimmune and inflammatory disorders including psoriasis, allergy, asthma, inflammatory bowel disease, rheumatoid arthritis and osteoarthritis.

Panel 1.2 Summary: Ag1536 Highest expression of this gene was detected in testis (CT=26.4). Therefore, antibody or small molecule therapies targeting encoded protein modulates testis function and is important in the treatment of diseases that affect the testis, including fertility and hypogonadism. Moderate to low expression of this gene was also detected in melanoma, pancreatic, brain, lung, breast, ovarian, renal, liver and colon cancer cell lines. Modulation of this gene and/or encoded protein is useful in the treatment of melanoma, pancreatic, brain, lung, breast, ovarian, renal, liver and colon cancers. Among tissues with metabolic or endocrine function, this gene was expressed at low levels in pancreas, adrenal gland, skeletal muscle, heart, liver and the gastrointestinal tract. Therefore, therapeutic modulation of the activity of this gene is useful in the treatment of endocrine/metabolically related diseases, such as obesity and diabetes. This gene was expressed at low levels in regions of the central nervous system including: amygdala, hippocampus, thalamus, cerebellum, and cerebral cortex. Therefore, therapeutic modulation of this gene and/or encoded protein is useful in the treatment of central nervous system disorders such as Alzheimer's disease, Parkinson's disease, epilepsy, multiple sclerosis, schizophrenia and depression.

Panel 2D Summary: Ag1536/Ag1589 Highest expression of this gene was detected in breast cancer and normal prostate samples (CTs=30-32). Significant expression of this gene was seen in normal and cancer samples from prostate, lung, kidney, breast, bladder. Therefore, therapeutic modulations of this gene and/or encoded protein is useful in the treatment of prostate, lung, kidney, breast, bladder cancers.

Panel 4D Summary: Ag1536/Ag1589 Highest expression of this gene was detected in activated small airway epithelium and IL-4 activated NCI-H292 (CTs=31-32). Moderate expression of this gene was also seen in resting keratinocytes, activated bronchial epithelium, resting and activated mucoepidermoid NCI-H292 cells, activated lung fibroblasts and liver cirrhosis sample. Therefore, therapeutic modulation of this gene and/or encoded protein is useful in the treatment of liver cirrhosis and inflammatory lung disorders that include chronic obstructive pulmonary disease, asthma, allergy and emphysema.

P. CG54254-04: Leucine-Rich Repeat Transmembrane Protein.

Expression of gene CG54254-04 was assessed using the primer-probe sets Ag148 and Ag201, described in Tables PA and PB. Results of the RTQ-PCR runs are shown in Tables PC, PD, PE, PF and PG.

TABLE PA

Probe Name Ag148

| | | | | |
|---|---|---|---|---|
| | | | Start | SEQ |
| Primers | Sequences | Length | Position | ID No |

| | | | | |
|---|---|---|---|---|
| Forward | 5′-cagccctggagcccaag-3′ | 17 | 1484 | 439 |
| Probe | TET-5′-ccacctacatcatctgcatggtcaccat-3′- | 28 | 1502 | 440 |
| | TAMRA | | | |
| Reverse | 5′-cgggtgtctcatcagctacgt-3′ | 21 | 1547 | 441 |

TABLE PB

Probe Name Ag201

| | | | | |
|---|---|---|---|---|
| | | | Start | SEQ |
| Primers | Sequence | Length | Position | ID No |

| | | | | |
|---|---|---|---|---|
| Forward | 5′-cagccctggagcccaag-3′ | 17 | 1484 | 442 |
| Probe | TET-5′-ccacctacatcatctgcatggtcacca-3′- | 27 | 1502 | 443 |
| | TAMRA | | | |
| Reverse | 5′-cgggtgtctcatcagctacgta-3′ | 22 | 1546 | 444 |

TABLE PC
CNS_neurodegeneration_v1.0

| Tissue Name | A | B | C |
|---|---|---|---|
| AD 1 Hippo | 24.1 | 15.7 | 0.0 |
| AD 2 Hippo | 39.2 | 21.8 | 34.6 |
| AD 3 Hippo | 20.0 | 9.9 | 9.5 |
| AD 4 Hippo | 25.5 | 15.2 | 30.6 |
| AD 5 Hippo | 63.7 | 85.3 | 47.0 |
| AD 6 Hippo | 50.7 | 29.9 | 26.4 |
| Control 2 Hippo | 51.8 | 27.2 | 38.2 |
| Control 4 Hippo | 21.8 | 17.0 | 30.4 |
| Control (Path) 3 Hippo | 10.3 | 5.0 | 9.3 |
| AD 1 Temporal Ctx | 21.6 | 18.4 | 25.9 |
| AD 2 Temporal Ctx | 24.7 | 15.8 | 24.8 |
| AD 3 Temporal Ctx | 8.7 | 7.2 | 10.4 |
| AD 4 Temporal Ctx | 25.5 | 13.6 | 27.9 |
| AD 5 Inf Temporal Ctx | 62.4 | 60.3 | 50.3 |
| AD 5 Sup Temporal Ctx | 47.3 | 66.0 | 32.3 |
| AD 6 Inf Temporal Ctx | 47.3 | 29.9 | 26.8 |
| AD 6 Sup Temporal Ctx | 35.6 | 24.3 | 26.6 |
| Control 1 Temporal Ctx | 17.2 | 15.7 | 21.6 |
| Control 2 Temporal Ctx | 61.1 | 23.8 | 44.4 |
| Control 3 Temporal Ctx | 35.4 | 17.6 | 21.9 |
| Control 3 Temporal Ctx | 20.0 | 13.9 | 19.3 |
| Control (Path) 1 Temporal | 89.5 | 46.3 | 81.8 |
| Ctx | | | |
| Control (Path) 2 Temporal | 52.9 | 25.7 | 40.1 |
| Ctx | | | |
| Control (Path) 3 Temporal | 6.3 | 7.9 | 6.0 |
| Ctx | | | |
| Control (Path) 4 Temporal | 29.7 | 24.0 | 29.7 |
| Ctx | | | |
| AD 1 Occipital Ctx | 20.2 | 7.1 | 15.2 |
| AD 2 Occipital Ctx | 0.0 | 0.0 | 0.0 |
| (Missing) | | | |
| AD 3 Occipital Ctx | 13.4 | 4.7 | 11.9 |
| AD 4 Occipital Ctx | 28.9 | 16.7 | 28.9 |
| AD 5 Occipital Ctx | 59.5 | 6.8 | 13.1 |
| AD 6 Occipital Ctx | 12.0 | 100.0 | 53.6 |
| Control 1 Occipital Ctx | 10.7 | 4.9 | 10.4 |
| Control 2 Occipital Ctx | 48.6 | 28.9 | 48.0 |
| Control 3 Occipital Ctx | 26.1 | 15.6 | 21.8 |
| Control 4 Occipital Ctx | 20.3 | 13.1 | 15.4 |
| Control (Path) 1 Occipital | 97.3 | 47.0 | 100.0 |
| Ctx | | | |
| Control (Path) 2 Occipital | 21.3 | 12.2 | 25.0 |
| Ctx | | | |
| Control (Path) 3 Occipital | 9.0 | 4.0 | 4.1 |
| Ctx | | | |
| Control (Path) 4 Occipital | 24.3 | 10.7 | 27.0 |
| Ctx | | | |
| Control 1 Parietal Ctx | 19.2 | 10.0 | 15.4 |
| Control 2 Parietal Ctx | 57.8 | 75.8 | 35.6 |
| Control 3 Parietal Ctx | 26.6 | 17.8 | 28.7 |
| Control (Path) 1 Parietal Ctx | 100.0 | 50.7 | 85.9 |
| Control (Path) 2 Parietal Ctx | 42.6 | 21.6 | 36.6 |
| Control (Path) 3 Parietal Ctx | 10.7 | 7.3 | 9.1 |
| Control (Path) 4 Parietal Ctx | 55.9 | 35.8 | 49.3 |

Column A - Rel. Exp. (%) Ag148, Run 206989705
Column B - Rel. Exp. (%) Ag148, Run 219923397
Column C - Rel. Exp. (%) Ag201, Run 206975347

TABLE PD
Panel 1

| Tissue Name | A |
|---|---|
| Endothelial cells | 2.6 |
| Endothelial cells (treated) | 0.1 |
| Pancreas | 9.7 |
| Pancreatic ca. CAPAN 2 | 2.6 |
| Adrenal gland | 6.8 |
| Thyroid | 14.4 |
| Salivary gland | 6.0 |
| Pituitary gland | 1.8 |
| Brain (fetal) | 11.9 |
| Brain (whole) | 61.6 |
| Brain (amygdala) | 28.1 |
| Brain (cerebellum) | 100.0 |
| Brain (hippocampus) | 18.0 |
| Brain (substantia nigra) | 16.7 |
| Brain (thalamus) | 19.5 |
| Brain (hypothalamus) | 2.9 |
| Spinal cord | 8.4 |
| glio/astro U87-MG | 1.8 |
| glio/astro U-118-MG | 2.0 |
| astrocytoma SW1783 | 0.5 |
| neuro*; met SK-N-AS | 1.9 |
| astrocytoma SF-539 | 2.1 |
| astrocytoma SNB-75 | 12.4 |
| glioma SNB-19 | 7.6 |
| glioma U251 | 1.6 |
| glioma SF-295 | 1.5 |
| Heart | 3.2 |
| Skeletal muscle | 3.8 |
| Bone marrow | 2.0 |
| Thymus | 5.8 |
| Spleen | 1.3 |
| Lymph node | 3.5 |
| Colon (ascending) | 4.9 |
| Stomach | 12.9 |
| Small intestine | 5.3 |
| Colon ca. SW480 | 2.8 |
| Colon ca.* SW620 (SW480 met) | 3.9 |
| Colon ca. HT29 | 6.4 |
| Colon ca. HCT-116 | 0.0 |
| Colon ca. CaCo-2 | 4.1 |
| Colon ca. HCT-15 | 5.3 |
| Colon ca. HCC-2998 | 4.9 |
| Gastric ca. * (liver met) NCI-N87 | 13.4 |
| Bladder | 3.4 |
| Trachea | 10.7 |
| Kidney | 15.4 |
| Kidney (fetal) | 12.1 |
| Renal ca. 786-0 | 1.3 |
| Renal ca. A498 | 2.8 |
| Renal ca. RXF 393 | 0.9 |
| Renal ca. ACHN | 1.5 |
| Renal ca. UO-31 | 0.9 |
| Renal ca. TK-10 | 5.6 |
| Liver | 2.7 |
| Liver (fetal) | 1.7 |
| Liver ca. (hepatoblast) HepG2 | 2.3 |
| Lung | 0.8 |
| Lung (fetal) | 2.7 |
| Lung ca. (small cell) LX-1 | 4.2 |
| Lung ca. (small cell) NCI-H69 | 11.6 |
| Lung ca. (s. cell var.) SHP-77 | 0.0 |
| Lung ca. (large cell) NCI-H460 | 0.0 |
| Lung ca. (non-sm. cell) A549 | 3.9 |
| Lung ca. (non-s. cell) NCI-H23 | 3.8 |
| Lung ca. (non-s. cell) HOP-62 | 1.5 |
| Lung ca. (non-s. cl) NCI-H522 | 5.8 |
| Lung ca. (squam.) SW 900 | 6.7 |
| Lung ca. (squam.) NCI-H596 | 7.9 |
| Mammary gland | 16.4 |
| Breast ca.* (pl. ef) MCF-7 | 17.7 |
| Breast ca.* (pl. ef) MDA-MB-231 | 4.4 |
| Breast ca.* (pl. ef) T47D | 16.7 |
| Breast ca. BT-549 | 0.0 |
| Breast ca. MDA-N | 17.1 |
| Ovary | 2.3 |
| Ovarian ca. OVCAR-3 | 5.9 |
| Ovarian ca. OVCAR-4 | 1.5 |
| Ovarian ca. OVCAR-5 | 8.7 |
| Ovarian ca. OVCAR-8 | 4.3 |
| Ovarian ca. IGROV-1 | 1.1 |
| Ovarian ca. (ascites) SK-OV-3 | 1.6 |
| Uterus | 31.4 |
| Placenta | 4.5 |
| Prostate | 9.0 |
| Prostate ca.* (bone met) PC-3 | 0.0 |
| Testis | 16.5 |
| Melanoma Hs688(A).T | 0.7 |
| Melanoma* (met) Hs688(B).T | 1.3 |
| Melanoma UACC-62 | 1.3 |
| Melanoma M14 | 3.3 |
| Melanoma LOX IMVI | 13.1 |
| Melanoma* (met) SK-MEL-5 | 1.3 |
| Melanoma SK-MEL-28 | 0.6 |

Column A - Rel. Exp. (%) Ag148, Run 87589460

TABLE PE
Panel 1.3D

| Tissue Name | A | B |
|---|---|---|
| Liver adenocarcinoma | 3.7 | 3.1 |
| Pancreas | 2.4 | 4.6 |
| Pancreatic ca. CAPAN 2 | 4.7 | 1.7 |
| Adrenal gland | 4.8 | 4.5 |
| Thyroid | 20.6 | 23.8 |
| Salivary gland | 1.0 | 2.8 |
| Pituitary gland | 4.5 | 2.0 |
| Brain (fetal) | 8.4 | 11.3 |
| Brain (whole) | 28.5 | 40.1 |
| Brain (amygdala) | 48.3 | 50.0 |
| Brain (cerebellum) | 7.5 | 7.3 |
| Brain (hippocampus) | 97.9 | 94.6 |
| Brain (substantia nigra) | 5.8 | 8.0 |
| Brain (thalamus) | 30.4 | 28.1 |
| Cerebral Cortex | 33.4 | 36.9 |
| Spinal cord | 6.4 | 10.2 |
| glio/astro U87-MG | 0.9 | 1.7 |
| glio/astro U-118-MG | 1.5 | 3.2 |
| astrocytoma SW1783 | 1.0 | 1.4 |
| neuro*; met SK-N-AS | 4.9 | 6.0 |
| astrocytoma SF-539 | 3.4 | 2.0 |
| astrocytoma SNB-75 | 5.8 | 10.6 |
| glioma SNB-19 | 3.4 | 4.0 |
| glioma U251 | 3.3 | 3.6 |
| glioma SF-295 | 9.3 | 10.7 |
| Heart (fetal) | 2.5 | 1.7 |
| Heart | 0.8 | 0.4 |
| Skeletal muscle (fetal) | 100.0 | 100.0 |
| Skeletal muscle | 0.3 | 0.3 |
| Bone marrow | 1.2 | 1.1 |
| Thymus | 3.9 | 2.1 |
| Spleen | 0.9 | 2.3 |
| Lymph node | 1.5 | 1.1 |
| Colorectal | 4.8 | 5.0 |
| Stomach | 4.7 | 9.0 |
| Small intestine | 5.8 | 6.9 |
| Colon ca. SW480 | 7.7 | 7.0 |
| Colon ca.* SW620(SW480 met) | 3.3 | 3.0 |
| Colon ca. HT29 | 0.6 | 2.5 |
| Colon ca. HCT-116 | 2.7 | 4.0 |
| Colon ca. CaCo-2 | 3.2 | 5.8 |
| Colon ca. tissue(ODO3866) | 0.5 | 1.0 |
| Colon ca. HCC-2998 | 2.4 | 3.4 |
| Gastric ca.* (liver met) NCI-N87 | 3.4 | 6.8 |
| Bladder | 1.8 | 3.4 |
| Trachea | 1.4 | 8.9 |
| Kidney | 2.8 | 6.6 |
| Kidney (fetal) | 4.6 | 6.4 |
| Renal ca. 786-0 | 1.5 | 0.8 |
| Renal ca. A498 | 7.3 | 8.2 |
| Renal ca. RXF 393 | 0.4 | 0.0 |
| Renal ca. ACHN | 0.9 | 1.2 |
| Renal ca. UO-31 | 2.0 | 1.1 |
| Renal ca. TK-10 | 3.3 | 6.7 |
| Liver | 0.9 | 0.0 |
| Liver (fetal) | 0.7 | 2.1 |
| Liver ca. (hepatoblast) HepG2 | 4.0 | 2.2 |
| Lung | 0.6 | 1.3 |
| Lung (fetal) | 0.7 | 2.4 |
| Lung ca. (small cell) LX-1 | 4.5 | 4.5 |
| Lung ca. (small cell) NCI-H69 | 19.1 | 18.8 |
| Lung ca. (s. cell var.) SHP-77 | 3.0 | 4.1 |
| Lung ca. (large cell)NCI-H460 | 3.6 | 4.9 |
| Lung ca. (non-sm. cell) A549 | 1.7 | 3.2 |
| Lung ca. (non-s. cell) NCI-H23 | 6.7 | 8.0 |
| Lung ca. (non-s. cell) HOP-62 | 5.4 | 1.8 |
| Lung ca. (non-s. cl) NCI-H522 | 4.8 | 4.8 |
| Lung ca. (squam.) SW 900 | 1.0 | 1.2 |
| Lung ca. (squam.) NCI-H596 | 4.6 | 3.4 |
| Mammary gland | 2.2 | 3.1 |
| Breast ca.* (pl. ef) MCF-7 | 3.1 | 3.3 |
| Breast ca.* (pl. ef) MDA-MB-231 | 1.8 | 2.4 |
| Breast ca.* (pl. ef) T47D | 1.6 | 5.2 |
| Breast ca. BT-549 | 1.7 | 1.2 |
| Breast ca. MDA-N | 4.4 | 7.5 |
| Ovary | 2.8 | 2.0 |
| Ovarian ca. OVCAR-3 | 0.7 | 3.3 |
| Ovarian ca. OVCAR-4 | 0.3 | 0.6 |
| Ovarian ca. OVCAR-5 | 4.1 | 3.9 |
| Ovarian ca. OVCAR-8 | 4.0 | 3.3 |
| Ovarian ca. IGROV-1 | 0.6 | 0.5 |
| Ovarian ca.* (ascites) SK-OV-3 | 1.6 | 0.3 |
| Uterus | 4.2 | 4.4 |
| Placenta | 3.5 | 3.1 |
| Prostate | 4.7 | 5.1 |
| Prostate ca.* (bone met)PC-3 | 1.8 | 2.9 |
| Testis | 1.8 | 2.8 |
| Melanoma Hs688(A).T | 1.5 | 2.6 |
| Melanoma* (met) Hs688(B).T | 4.9 | 5.6 |
| Melanoma UACC-62 | 1.4 | 0.0 |
| Melanoma M14 | 0.4 | 0.9 |
| Melanoma LOX IMVI | 1.4 | 1.3 |
| Melanoma* (met) SK-MEL-5 | 1.8 | 1.2 |
| Adipose | 0.0 | 0.6 |

Column A - Rel. Exp. (%) Ag148, Run 150018164
Column B - Rel. Exp. (%) Ag201, Run 152827283

TABLE PF
Panel 2D

| Tissue Name | A | B | C |
|---|---|---|---|
| Normal Colon | 40.1 | 59.9 | 51.8 |
| CC Well to Mod Diff | 16.2 | 11.6 | 6.6 |
| (ODO3866) | | | |
| CC Margin (ODO3866) | 14.8 | 25.9 | 12.0 |
| CC Gr.2 rectosigmoid | 2.3 | 6.4 | 4.3 |
| (ODO3868) | | | |
| CC Margin (ODO3868) | 14.3 | 16.8 | 13.2 |
| CC Mod Diff (ODO3920) | 11.5 | 42.0 | 26.8 |
| CC Margin (ODO3920) | 13.3 | 25.5 | 21.5 |
| CC Gr.2 ascend colon | 6.7 | 30.6 | 14.9 |
| (ODO3921) | | | |
| CC Margin (ODO3921) | 7.6 | 17.4 | 15.0 |
| CC from Partial Hepatectomy | 2.4 | 15.8 | 11.7 |
| (ODO4309) Mets | | | |
| Liver Margin (ODO4309) | 0.3 | 2.0 | 3.1 |
| Colon mets to lung (OD04451- | 2.0 | 0.0 | 7.6 |
| 01) | | | |
| Lung Margin (OD04451-02) | 1.0 | 3.7 | 0.0 |
| Normal Prostate 6546-1 | 20.3 | 44.1 | 21.8 |
| Prostate Cancer (OD04410) | 18.7 | 47.6 | 41.5 |
| Prostate Margin (OD04410) | 9.8 | 25.2 | 17.7 |
| Prostate Cancer (OD04720-01) | 21.0 | 35.6 | 17.1 |
| Prostate Margin (OD04720-02) | 10.4 | 23.0 | 14.3 |
| Normal Lung 061010 | 9.6 | 27.2 | 14.1 |
| Lung Met to Muscle | 14.0 | 17.6 | 13.0 |
| (ODO4286) | | | |
| Muscle Margin (ODO4286) | 9.5 | 21.2 | 37.1 |
| Lung Malignant Cancer | 0.3 | 5.8 | 10.6 |
| (OD03126) | | | |
| Lung Margin (OD03126) | 1.2 | 6.3 | 2.3 |
| Lung Cancer (OD04404) | 1.2 | 1.5 | 0.0 |
| Lung Margin (OD04404) | 4.5 | 5.5 | 3.9 |
| Lung Cancer (OD04565) | 1.6 | 3.0 | 0.9 |
| Lung Margin (OD04565) | 1.9 | 2.9 | 1.1 |
| Lung Cancer (OD04237-01) | 5.6 | 16.6 | 3.1 |
| Lung Margin (OD04237-02) | 0.8 | 3.1 | 0.9 |
| Ocular Mel Met to Liver | 8.0 | 7.1 | 7.8 |
| (ODO4310) | | | |
| Liver Margin (ODO4310) | 2.6 | 3.1 | 0.4 |
| Melanoma Mets to Lung | 10.0 | 18.6 | 21.0 |
| (OD04321) | | | |
| Lung Margin (OD04321) | 0.3 | 5.6 | 1.2 |
| Normal Kidney | 29.3 | 75.3 | 100.0 |
| Kidney Ca, Nuclear grade 2 | 2.7 | 7.0 | 13.5 |
| (OD04338) | | | |
| Kidney Margin (OD04338) | 39.0 | 44.4 | 65.1 |
| Kidney Ca Nuclear grade 1/2 | 6.3 | 9.2 | 9.0 |
| (OD04339) | | | |
| Kidney Margin (OD04339) | 28.9 | 51.1 | 42.9 |
| Kidney Ca, Clear cell type | 11.4 | 9.2 | 5.7 |
| (OD04340) | | | |
| Kidney Margin (OD04340) | 73.2 | 90.1 | 95.9 |
| Kidney Ca, Nuclear grade 3 | 3.7 | 4.3 | 3.8 |
| (OD04348) | | | |
| Kidney Margin (OD04348) | 25.5 | 63.7 | 58.6 |
| Kidney Cancer (OD04622-01) | 0.5 | 3.0 | 0.3 |
| Kidney Margin (OD04622-03) | 19.2 | 21.0 | 9.7 |
| Kidney Cancer (OD04450-01) | 1.8 | 3.3 | 0.8 |
| Kidney Margin (OD04450-03) | 34.6 | 46.7 | 53.2 |
| Kidney Cancer 8120607 | 0.8 | 2.6 | 3.4 |
| Kidney Margin 8120608 | 23.8 | 25.2 | 28.9 |
| Kidney Cancer 8120613 | 7.0 | 25.9 | 25.3 |
| Kidney Margin 8120614 | 12.4 | 39.0 | 23.3 |
| Kidney Cancer 9010320 | 2.0 | 4.4 | 1.0 |
| Kidney Margin 9010321 | 26.4 | 26.1 | 25.9 |
| Normal Uterus | 3.0 | 7.7 | 2.5 |
| Uterus Cancer 064011 | 8.3 | 16.3 | 14.3 |
| Normal Thyroid | 68.3 | 100.0 | 55.9 |
| Thyroid Cancer 064010 | 1.4 | 8.8 | 5.8 |
| Thyroid Cancer A302152 | 6.0 | 7.4 | 2.5 |
| Thyroid Margin A302153 | 15.2 | 36.6 | 39.8 |
| Normal Breast | 8.3 | 23.7 | 26.2 |
| Breast Cancer (OD04566) | 4.1 | 5.6 | 13.6 |
| Breast Cancer (OD04590- | 17.2 | 13.6 | 23.7 |
| 01) | | | |
| Breast Cancer Mets | 20.0 | 26.8 | 25.7 |
| (OD04590-03) | | | |
| Breast Cancer Metastasis | 18.9 | 7.7 | 7.6 |
| (OD04655-05) | | | |
| Breast Cancer 064006 | 8.1 | 14.7 | 20.9 |
| Breast Cancer 1024 | 100.0 | 100.0 | 59.5 |
| Breast Cancer 9100266 | 8.7 | 17.9 | 17.2 |
| Breast Margin 9100265 | 4.3 | 16.0 | 7.1 |
| Breast Cancer A209073 | 4.0 | 20.2 | 17.2 |
| Breast Margin A209073 | 4.1 | 18.0 | 18.8 |
| Normal Liver | 4.4 | 10.3 | 6.5 |
| Liver Cancer 064003 | 3.9 | 1.6 | 0.0 |
| Liver Cancer 1025 | 5.1 | 10.6 | 6.7 |
| Liver Cancer 1026 | 0.0 | 0.3 | 6.5 |
| Liver Cancer 6004-T | 4.9 | 11.0 | 13.5 |
| Liver Tissue 6004-N | 24.5 | 26.4 | 29.3 |
| Liver Cancer 6005-T | 2.9 | 3.1 | 1.9 |
| Liver Tissue 6005-N | 0.0 | 2.0 | 0.0 |
| Normal Bladder | 4.7 | 19.1 | 14.4 |
| Bladder Cancer 1023 | 1.9 | 9.1 | 11.2 |
| Bladder Cancer A302173 | 0.3 | 13.6 | 5.7 |
| Bladder Cancer | 2.7 | 6.3 | 4.1 |
| (OD04718-01) | | | |
| Bladder Normal Adjacent | 1.7 | 5.9 | 4.5 |
| (OD04718-03) | | | |
| Normal Ovary | 4.3 | 9.3 | 2.0 |
| Ovarian Cancer 064008 | 4.3 | 12.2 | 14.5 |
| Ovarian Cancer | 11.0 | 11.2 | 13.2 |
| (OD04768-07) | | | |
| Ovary Margin (OD04768- | 1.1 | 3.6 | 0.8 |
| 08) | | | |
| Normal Stomach | 33.0 | 16.3 | 15.2 |
| Gastric Cancer 9060358 | 5.1 | 1.0 | 2.8 |
| Stomach Margin 9060359 | 2.5 | 5.8 | 6.3 |
| Gastric Cancer 9060395 | 7.8 | 11.9 | 11.9 |
| Stomach Margin 9060394 | 3.4 | 10.0 | 7.3 |
| Gastric Cancer 9060397 | 18.3 | 28.1 | 9.8 |
| Stomach Margin 9060396 | 1.5 | 3.0 | 4.1 |
| Gastric Cancer 064005 | 2.1 | 11.7 | 8.6 |

Column A - Rel. Exp. (%) Ag148, Run 147664547
Column B - Rel. Exp. (%) Ag148, Run 150018225
Column C - Rel. Exp. (%) Ag201, Run 152827305

TABLE PG
Panel 4D

| Tissue Name | A | B | C | D |
|---|---|---|---|---|
| Secondary Th1 act | 9.3 | 20.2 | 15.9 | 25.0 |
| Secondary Th2 act | 12.5 | 12.2 | 14.8 | 20.2 |
| Secondary Tr1 act | 16.7 | 11.4 | 19.8 | 25.2 |
| Secondary Th1 rest | 8.9 | 3.5 | 0.0 | 1.3 |
| Secondary Th2 rest | 6.7 | 3.5 | 7.1 | 3.2 |
| Secondary Tr1 rest | 10.5 | 6.6 | 12.8 | 1.5 |
| Primary Th1 act | 26.8 | 11.7 | 13.1 | 14.4 |
| Primary Th2 act | 17.2 | 5.6 | 24.1 | 6.8 |
| Primary Tr1 act | 32.8 | 7.6 | 22.7 | 7.0 |
| Primary Th1 rest | 17.7 | 5.8 | 26.6 | 14.8 |
| Primary Th2 rest | 8.8 | 1.8 | 13.0 | 0.6 |
| Primary Tr1 rest | 22.8 | 5.6 | 17.1 | 1.1 |
| CD45RA CD4 lymphocyte act | 7.9 | 5.8 | 10.7 | 28.1 |
| CD45RO CD4 lymphocyte act | 15.5 | 24.3 | 22.8 | 24.7 |
| CD8 lymphocyte act | 22.5 | 9.1 | 19.8 | 10.7 |
| Secondary CD8 lymphocyte rest | 8.7 | 16.0 | 13.7 | 16.0 |
| Secondary CD8 lymphocyte act | 4.9 | 1.6 | 9.4 | 4.7 |
| CD4 lymphocyte none | 3.7 | 0.0 | 0.0 | 3.6 |
| 2ry Th1/Th2/Tr1_anti-CD95 CH11 | 3.3 | 2.7 | 2.7 | 0.3 |
| LAK cells rest | 9.0 | 4.9 | 4.5 | 3.0 |
| LAK cells IL-2 | 8.4 | 2.4 | 1.3 | 3.0 |
| LAK cells IL-2 + IL-12 | 10.0 | 6.3 | 5.1 | 6.5 |
| LAK cells IL-2 + IFN gamma | 6.3 | 7.0 | 12.6 | 0.0 |
| LAK cells IL-2 + IL-18 | 11.3 | 10.3 | 9.4 | 4.4 |
| LAK cells PMA/ionomycin | 0.0 | 7.7 | 4.2 | 3.8 |
| NK Cells IL-2 rest | 2.0 | 2.1 | 3.0 | 6.9 |
| Two Way MLR 3 day | 12.4 | 17.3 | 5.5 | 15.0 |
| Two Way MLR 5 day | 2.0 | 7.6 | 3.0 | 5.3 |
| Two Way MLR 7 day | 1.8 | 1.5 | 3.7 | 10.6 |
| PBMC rest | 1.8 | 1.3 | 1.8 | 0.0 |
| PBMC PWM | 32.3 | 12.6 | 22.7 | 13.8 |
| PBMC PHA-L | 8.2 | 1.6 | 6.7 | 2.5 |
| Ramos (B cell) none | 12.6 | 10.8 | 18.2 | 2.6 |
| Ramos (B cell) ionomycin | 59.0 | 13.7 | 47.0 | 21.3 |
| B lymphocytes PWM | 18.2 | 5.6 | 19.8 | 13.7 |
| B lymphocytes CD40L and IL-4 | 11.5 | 6.2 | 10.2 | 1.5 |
| EOL-1 dbcAMP | 46.3 | 58.6 | 26.8 | 39.8 |
| EOL-1 dbcAMP PMA/ionomycin | 5.6 | 14.7 | 16.7 | 21.3 |
| Dendritic cells none | 2.0 | 4.0 | 6.6 | 6.5 |
| Dendritic cells LPS | 0.0 | 5.4 | 1.0 | 0.0 |
| Dendritic cells anti-CD40 | 1.5 | 7.1 | 2.2 | 6.4 |
| Monocytes rest | 0.0 | 0.0 | 1.1 | 0.0 |
| Monocytes LPS | 0.0 | 3.2 | 0.0 | 0.0 |
| Macrophages rest | 7.2 | 1.8 | 5.7 | 4.7 |
| Macrophages LPS | 0.0 | 4.5 | 2.3 | 0.0 |
| HUVEC none | 18.2 | 17.3 | 5.3 | 3.5 |
| HUVEC starved | 17.4 | 0.4 | 12.6 | 8.7 |
| HUVEC IL-1beta | 7.2 | 1.3 | 9.4 | 13.2 |
| HUVEC IFN gamma | 7.7 | 9.1 | 4.9 | 7.2 |
| HUVEC TNF alpha + IFN gamma | 5.3 | 2.8 | 5.8 | 2.4 |
| HUVEC TNF alpha + IL4 | 15.0 | 14.2 | 11.3 | 12.6 |
| HUVEC IL-11 | 5.6 | 7.1 | 4.0 | 1.8 |
| Lung Microvascular EC none | 16.0 | 11.3 | 24.0 | 15.5 |
| Lung Microvascular EC | 12.9 | 9.8 | 15.6 | 6.9 |
| TNFalpha + IL-1beta | | | | |
| Microvascular Dermal EC none | 7.0 | 5.8 | 6.1 | 7.3 |
| Microsvasular Dermal EC | 1.0 | 8.8 | 6.7 | 7.6 |
| TNFalpha + IL-1beta | | | | |
| Bronchial epithelium | 10.3 | 0.2 | 0.0 | 1.8 |
| TNFalpha + IL1beta | | | | |
| Small airway epithelium none | 4.6 | 3.8 | 0.0 | 3.3 |
| Small airway epithelium | 8.1 | 4.2 | 5.3 | 0.3 |
| TNFalpha + IL-1beta | | | | |
| Coronery artery SMC rest | 0.0 | 1.1 | 4.4 | 2.2 |
| Coronery artery SMC | 2.3 | 1.4 | 4.6 | 6.7 |
| TNFalpha + IL-1beta | | | | |
| Astrocytes rest | 7.6 | 7.3 | 7.0 | 11.0 |
| Astrocytes TNFalpha + IL-1beta | 4.0 | 13.2 | 0.8 | 7.3 |
| KU-812 (Basophil) rest | 68.8 | 63.7 | 54.7 | 60.7 |
| KU-812 (Basophil) PMA/ionomycin | 47.6 | 55.1 | 47.6 | 21.3 |
| CCD1106 (Keratinocytes) none | 16.2 | 11.3 | 22.8 | 17.7 |
| CCD1106 (Keratinocytes) | 3.4 | 0.0 | 1.5 | 4.1 |
| TNFalpha + IL-1beta | | | | |
| Liver cirrhosis | 4.3 | 9.5 | 13.9 | 13.1 |
| Lupus kidney | 11.3 | 1.6 | 3.1 | 10.4 |
| NCI-H292 none | 43.8 | 16.0 | 36.6 | 5.6 |
| NCI-H292 IL-4 | 95.3 | 28.5 | 70.7 | 39.5 |
| NCI-H292 IL-9 | 95.9 | 23.2 | 65.1 | 50.7 |
| NCI-H292 IL-13 | 41.8 | 100.0 | 53.6 | 100.0 |
| NCI-H292 IFN gamma | 52.5 | 52.5 | 39.0 | 95.3 |
| HPAEC none | 9.5 | 8.9 | 7.6 | 15.1 |
| HPAEC TNF alpha + IL-1 beta | 12.0 | 12.9 | 5.0 | 11.1 |
| Lung fibroblast none | 4.0 | 8.9 | 7.3 | 9.1 |
| Lung fibroblast TNF | 1.7 | 1.0 | 3.3 | 1.4 |
| alpha + IL-1 beta | | | | |
| Lung fibroblast IL-4 | 14.0 | 5.2 | 4.7 | 6.7 |
| Lung fibroblast IL-9 | 12.6 | 7.9 | 3.6 | 6.1 |
| Lung fibroblast IL-13 | 4.3 | 5.3 | 3.2 | 6.0 |
| Lung fibroblast IFN gamma | 8.2 | 4.0 | 1.1 | 1.8 |
| Dermal fibroblast CCD1070 rest | 25.0 | 7.5 | 21.8 | 14.3 |
| Dermal fibroblast CCD1070 TNF alpha | 44.4 | 2.5 | 19.6 | 9.3 |
| Dermal fibroblast CCD1070 IL-1 beta | 5.8 | 20.6 | 11.2 | 6.2 |
| Dermal fibroblast IFN gamma | 11.3 | 10.2 | 1.3 | 8.9 |
| Dermal fibroblast IL-4 | 5.7 | 6.7 | 8.7 | 18.3 |
| IBD Colitis 2 | 0.0 | 1.3 | 1.3 | 1.7 |
| IBD Crohn's | 1.9 | 0.0 | 0.0 | 5.5 |
| Colon | 40.6 | 26.2 | 42.3 | 52.5 |
| Lung | 9.0 | 9.1 | 27.7 | 16.7 |
| Thymus | 100.0 | 55.9 | 100.0 | 57.4 |
| Kidney | 9.7 | 8.7 | 6.7 | 14.4 |

Column A - Rel. Exp. (%) Ag148, Run 150048886
Column B - Rel. Exp. (%) Ag148, Run 152784557
Column C - Rel. Exp. (%) Ag201, Run 152827367
Column D - Rel. Exp. (%) Ag201, Run 157871331

CNS_neurodegeneration_v1.0 Summary: Ag148/Ag201 This gene was down-regulated in the temporal cortex of Alzheimer's disease patients. Therefore, up-regulation of this gene, expressed protein, and/or treatment with specific agonists targeting encoded protein is useful in reversing the dementia/memory loss associated with this disease and neuronal death. Panel 1 Summary: Ag148 Highest expression of this gene was detected in cerebellum (CT=24.5) and this gene was expressed at high levels in all regions of the central nervous system examined including: amygdala, hippocampus, substantia nigra, thalamus, cerebellum, cerebral cortex, and spinal cord. Therefore, therapeutic modulation of this gene and/or expressed protein is useful in the diagnosis and treatment of central nervous system disorders such as Alzheimer's disease, Parkinson's disease, epilepsy, multiple sclerosis, schizophrenia and depression. Moderate gene expression levels was also seen in pancreatic, gastric, colon, lung, liver, renal, breast, ovarian, melanoma and brain cancer cell lines. Thus, expression level of this gene is a useful marker to detect the presence of these cancers. Furthermore, therapeutic modulation of the expression or function of this gene is an effective in the treatment of pancreatic, gastric, colon, lung, liver, renal, breast, ovarian, melanoma and brain cancers. Among tissues with metabolic or endocrine function, this gene was expressed at moderate to low levels in pancreas, adrenal gland, thyroid, pituitary gland, skeletal muscle, heart, liver and the gastrointestinal tract. Therefore, therapeutic modulation of the activity of this gene is useful in the treatment of endocrine/metabolically related diseases, such as obesity and diabetes.

Panel 1.3D Summary: Ag148/Ag201 Highest expression of this gene was seen in fetal skeletal muscle (CTs=30). Expression of this gene was higher in fetal compared adult skeletal muscle (CTs=38). The relative overexpression of this gene in fetal skeletal muscle indicates that the protein product enhances muscular growth or development and has regenerative capacity in the adult. Therefore, therapeutic modulation of this gene, expressed protein and/or use of antibodies or small molecule drugs targeting the gene or gene product are useful in treatment of muscle related diseases. More specifically, treatment of weak or dystrophic muscle with the protein encoded by this gene restores muscle mass or function. Moderate expression of this gene was seen in all the regions of the brain examined including: amygdala, hippocampus, substantia nigra, thalamus, cerebellum, cerebral cortex, and spinal cord. Modulation of this gene, the expressed protein and/or use of antibodies or small molecule drugs targeting the gene or gene product are useful in the treatment of central nervous system disorders such as Alzheimer's disease, Parkinson's disease, epilepsy, multiple sclerosis, schizophrenia and depression.

Panel 2D Summary: Ag148/Ag201 Highest expression of this gene was seen in breast cancer and normal kidney samples (CTs=30-31). Moderate to low expression of this gene was also seen in normal and cancer samples from stomach, ovary, liver, breast, kidney, metastatic melanoma, lung, prostate and colon. Expression of this gene is consistently higher in normal kidney compared to the corresponding cancer samples. This gene codes for fibronectin leucine repeat transmembrane protein 1 (FLRT1) acts as a matrix adhesion molecule or cell-cell adhesion molecule. This gene or encoded FLRT1 protein inhibits the growth of kidney cancer cells.

Panel 4D Summary: Ag148/Ag201 Highest expression of this gene was detected in IL13 activated NCI-NCI-H292 cells and thymus (CTs=31). This gene showed low wide spread expression in this panel with higher expression in resting and activated mucoepidermoid NCI-NCI-H292 cells, activated dermal fibroblasts, resting and activated basophils, eosinophils, PBMC cells, activated B lymphocytes and normal colon. Therefore, modulation of the gene and/or encoded protein alters functions associated with these cell types and leads to improvement of the symptoms of patients suffering from autoimmune and inflammatory diseases such as asthma, allergies, inflammatory bowel disease, lupus erythematosus, psoriasis, rheumatoid arthritis, and osteoarthritis.

Q. CG96778-01: Human Medium-Chain acyl-CoA Dehydrogenase.

Expression of gene CG96778-01 was assessed using the Primer-probe set Ag4326, described in Table QA. Results of the RTQ-PCR runs are shown in Table QB.

TABLE QA

Probe Name Ag4326

| | | | | |
|---|---|---|---|---|
| | | | Start | SEQ |
| Primers | Sequences | Length | Position | ID No |

| | | | | |
|---|---|---|---|---|
| Forward | 5′-catatgggtgattttgtgcttt-3′ | 22 | 138 | 445 |
| Probe | TET-5′-cgttcatcttttctgtgtttaaaatgttca-3′- | 30 | 161 | 446 |
| | TAMRA | | | |
| Reverse | 5′-ttcatgtctcctttgttccaat-3′ | 22 | 200 | 447 |

TABLE QB
General_screening_panel_v1.4

| Tissue Name | A |
|---|---|
| Adipose | 0.0 |
| Melanoma* Hs688(A).T | 2.2 |
| Melanoma* Hs688(B).T | 5.3 |
| Melanoma* M14 | 26.8 |
| Melanoma* LOXIMVI | 27.0 |
| Melanoma* SK-MEL-5 | 16.2 |
| Squamous cell carcinoma SCC-4 | 41.8 |
| Testis Pool | 21.6 |
| Prostate ca.* (bone met) PC-3 | 4.5 |
| Prostate Pool | 2.0 |
| Placenta | 3.4 |
| Uterus Pool | 0.0 |
| Ovarian ca. OVCAR-3 | 44.8 |
| Ovarian ca. SK-OV-3 | 8.4 |
| Ovarian ca. OVCAR-4 | 11.6 |
| Ovarian ca. OVCAR-5 | 33.0 |
| Ovarian ca. IGROV-1 | 13.0 |
| Ovarian ca. OVCAR-8 | 5.8 |
| Ovary | 4.7 |
| Breast ca. MCF-7 | 8.7 |
| Breast ca. MDA-MB-231 | 64.6 |
| Breast ca. BT 549 | 22.5 |
| Breast ca. T47D | 86.5 |
| Breast ca. MDA-N | 4.4 |
| Breast Pool | 0.0 |
| Trachea | 4.6 |
| Lung | 0.0 |
| Fetal Lung | 0.0 |
| Lung ca. NCI-N417 | 2.5 |
| Lung ca. LX-1 | 47.6 |
| Lung ca. NCI-H146 | 13.8 |
| Lung ca. SHP-77 | 11.1 |
| Lung ca. A549 | 6.2 |
| Lung ca. NCI-H526 | 3.1 |
| Lung ca. NCI-H23 | 37.1 |
| Lung ca. NCI-H460 | 4.7 |
| Lung ca. HOP-62 | 5.8 |
| Lung ca. NCI-H522 | 40.9 |
| Liver | 0.0 |
| Fetal Liver | 4.5 |
| Liver ca. HepG2 | 25.2 |
| Kidney Pool | 5.0 |
| Fetal Kidney | 4.1 |
| Renal ca. 786-0 | 32.3 |
| Renal ca. A498 | 7.7 |
| Renal ca. ACHN | 0.0 |
| Renal ca. UO-31 | 13.6 |
| Renal ca. TK-10 | 20.7 |
| Bladder | 6.0 |
| Gastric ca. (liver met.) NCI-N87 | 100.0 |
| Gastric ca. KATO III | 0.0 |
| Colon ca. SW-948 | 8.8 |
| Colon ca. SW480 | 45.4 |
| Colon ca.* (SW480 met) SW620 | 39.5 |
| Colon ca. HT29 | 17.0 |
| Colon ca. HCT-116 | 30.4 |
| Colon ca. CaCo-2 | 7.1 |
| Colon cancer tissue | 0.0 |
| Colon ca. SW1116 | 2.2 |
| Colon ca. Colo-205 | 3.4 |
| Colon ca. SW-48 | 13.3 |
| Colon Pool | 0.0 |
| Small Intestine Pool | 2.7 |
| Stomach Pool | 0.0 |
| Bone Marrow Pool | 0.0 |
| Fetal Heart | 0.0 |
| Heart Pool | 0.0 |
| Lymph Node Pool | 0.0 |
| Fetal Skeletal Muscle | 0.0 |
| Skeletal Muscle Pool | 3.8 |
| Spleen Pool | 5.9 |
| Thymus Pool | 6.2 |
| CNS cancer (glio/astro) U87-MG | 18.3 |
| CNS cancer (glio/astro) U-118-MG | 71.7 |
| CNS cancer (neuro; met) SK-N-AS | 24.8 |
| CNS cancer (astro) SF-539 | 12.8 |
| CNS cancer (astro) SNB-75 | 50.0 |
| CNS cancer (glio) SNB-19 | 16.6 |
| CNS cancer (glio) SF-295 | 10.4 |
| Brain (Amygdala) Pool | 4.2 |
| Brain (cerebellum) | 21.6 |
| Brain (fetal) | 6.1 |
| Brain (Hippocampus) Pool | 15.3 |
| Cerebral Cortex Pool | 9.4 |
| Brain (Substantia nigra) Pool | 6.6 |
| Brain (Thalamus) Pool | 9.5 |
| Brain (whole) | 3.3 |
| Spinal Cord Pool | 7.7 |
| Adrenal Gland | 13.6 |
| Pituitary gland Pool | 10.7 |
| Salivary Gland | 0.0 |
| Thyroid (female) | 2.2 |
| Pancreatic ca. CAPAN2 | 34.9 |
| Pancreas Pool | 5.3 |

Column A - Rel. Exp. (%) Ag4326, Run 222377145

General_screening_panel_v1.4 Summary: Ag4326 Highest expression of this gene was mainly seen in NCI-N87 gastric cancer cell line (CT=34.1). Low expression of this gene was also seen in brain and breast cancer cell lines. Therefore, expression level of this gene is a usedful marker to detect the presence of gastric, brain and breast cancers. In addition, therapeutic modulation of this gene and/or encoded protein is useful in the treatment of these cancers.

R. CG96778-02: Medium-Chain acyl-CoA Dehydrogenase.

Expression of gene CG96778-02 was assessed using the primer-probe set Ag6978, described in Table RA. Results of the RTQ-PCR runs are shown in Table RB. This sequence represents a physical full length clone.

TABLE RA

Probe Name Ag6978

| | | | | |
|---|---|---|---|---|
| | | | Start | SEQ |
| Primers | Sequences | Length | Position | ID No |

| | | | | |
|---|---|---|---|---|
| Forward | 5′-acttggtttaatgaacacacacatt-3′ | 25 | 249 | 448 |
| Probe | TET-5′-ccagagaactgtgactacagtgtttgccc-3′- | 29 | 274 | 449 |
| | TAMRA | | | |
| Reverse | 5′-gtatagagtgcaagcttccaaaagt-3′ | 25 | 303 | 450 |

TABLE RB
General_screening_panel_v1.6

| Tissue Name | A |
|---|---|
| Adipose | 0.0 |
| Melanoma* Hs688(A).T | 0.0 |
| Melanoma* Hs688(B).T | 0.0 |
| Melanoma* M14 | 0.0 |
| Melanoma* LOXIMVI | 0.0 |
| Melanoma* SK-MEL-5 | 0.0 |
| Squamous cell carcinoma SCC-4 | 0.0 |
| Testis Pool | 0.6 |
| Prostate ca.* (bone met) PC-3 | 0.0 |
| Prostate Pool | 0.0 |
| Placenta | 0.0 |
| Uterus Pool | 0.0 |
| Ovarian ca. OVCAR-3 | 0.0 |
| Ovarian ca. SK-OV-3 | 0.0 |
| Ovarian ca. OVCAR-4 | 0.0 |
| Ovarian ca. OVCAR-5 | 0.0 |
| Ovarian ca. IGROV-1 | 0.0 |
| Ovarian ca. OVCAR-8 | 0.0 |
| Ovary | 0.0 |
| Breast ca. MCF-7 | 0.0 |
| Breast ca. MDA-MB-231 | 0.6 |
| Breast ca. BT 549 | 0.0 |
| Breast ca. T47D | 0.0 |
| Breast ca. MDA-N | 0.0 |
| Breast Pool | 0.4 |
| Trachea | 0.0 |
| Lung | 0.5 |
| Fetal Lung | 0.0 |
| Lung ca. NCI-N417 | 0.0 |
| Lung ca. LX-1 | 0.0 |
| Lung ca. NCI-H146 | 0.7 |
| Lung ca. SHP-77 | 0.8 |
| Lung ca. A549 | 0.0 |
| Lung ca. NCI-H526 | 0.0 |
| Lung ca. NCI-H23 | 0.2 |
| Lung ca. NCI-H460 | 0.0 |
| Lung ca. HOP-62 | 0.0 |
| Lung ca. NCI-H522 | 0.0 |
| Liver | 0.0 |
| Fetal Liver | 0.0 |
| Liver ca. HepG2 | 0.4 |
| Kidney Pool | 0.4 |
| Fetal Kidney | 0.5 |
| Renal ca. 786-0 | 0.0 |
| Renal ca. A498 | 0.0 |
| Renal ca. ACHN | 0.0 |
| Renal ca. UO-31 | 0.5 |
| Renal ca. TK-10 | 0.0 |
| Bladder | 0.0 |
| Gastric ca. (liver met.) NCI-N87 | 0.0 |
| Gastric ca. KATO III | 0.0 |
| Colon ca. SW-948 | 0.0 |
| Colon ca. SW480 | 0.1 |
| Colon ca.* (SW480 met) SW620 | 0.6 |
| Colon ca. HT29 | 0.0 |
| Colon ca. HCT-116 | 0.0 |
| Colon ca. CaCo-2 | 0.0 |
| Colon cancer tissue | 0.0 |
| Colon ca. SW1116 | 0.0 |
| Colon ca. Colo-205 | 0.0 |
| Colon ca. SW-48 | 0.0 |
| Colon Pool | 0.0 |
| Small Intestine Pool | 0.0 |
| Stomach Pool | 0.0 |
| Bone Marrow Pool | 0.0 |
| Fetal Heart | 4.2 |
| Heart Pool | 5.6 |
| Lymph Node Pool | 0.6 |
| Fetal Skeletal Muscle | 3.0 |
| Skeletal Muscle Pool | 0.0 |
| Spleen Pool | 0.0 |
| Thymus Pool | 0.0 |
| CNS cancer (glio/astro) U87-MG | 0.0 |
| CNS cancer (glio/astro) U-118-MG | 0.0 |
| CNS cancer (neuro; met) SK-N-AS | 0.6 |
| CNS cancer (astro) SF-539 | 0.0 |
| CNS cancer (astro) SNB-75 | 0.0 |
| CNS cancer (glio) SNB-19 | 0.0 |
| CNS cancer (glio) SF-295 | 0.0 |
| Brain (Amygdala) Pool | 4.5 |
| Brain (cerebellum) | 100.0 |
| Brain (fetal) | 12.2 |
| Brain (Hippocampus) Pool | 3.8 |
| Cerebral Cortex Pool | 5.7 |
| Brain (Substantia nigra) Pool | 8.8 |
| Brain (Thalamus) Pool | 9.3 |
| Brain (whole) | 11.9 |
| Spinal Cord Pool | 3.0 |
| Adrenal Gland | 0.0 |
| Pituitary gland Pool | 0.0 |
| Salivary Gland | 0.0 |
| Thyroid (female) | 0.4 |
| Pancreatic ca. CAPAN2 | 0.0 |
| Pancreas Pool | 0.0 |

Column A - Rel. Exp. (%) Ag6978, Run 279065832

General_screening_panel_v1.6 Summary: Ag6978 Highest expression of this gene was seen in cerebellum (CT=29.6). Moderate to low expression of this gene was seen in all the regions of central nervous system examined including: amygdala, hippocampus, substantia nigra, thalamus, cerebellum, cerebral cortex, and spinal cord. Therefore, therapeutic modulation of this gene and/or expressed protein is useful in the treatment of central nervous system disorders such as Alzheimer's disease, Parkinson's disease, epilepsy, multiple sclerosis, schizophrenia and depression. Low expression of this gene was also seen in fetal and adult heart. Therefore, modulation of this gene and/or encoded protein will be useful in the treatment of heart related diseases.

### Example D

### Expression Data

### Example D1

### Expression of CG50949-07 in Stable CHO-K1 Cells

A 2548 bp long BamHI-XhoI fragment containing the CG52643-05 sequence was subcloned into BamHI-XhoI digested pEE14.4/Sec to generate plasmid 2134. The resulting plasmid 2134 was transfected into CHO-K1 cells using the LipofectaminePlus reagent following the manufacturer's instructions (Invitrogen/Gibco Stable clones were selected based on resistance against methionine sulfoximine. The expression and secretion levels of the selected clones were assessed by Western blot analysis using HRP conjugated V5 antibody. (The V5 epitope is fused to the gene of interest at the Cter, in the pEE14.4Sec vector.) The CG50949-07 protein is secreted as a 55 kDa protein.

### Example D2

### Expression of CG52643-05 Using Baculovirus Expression System

A 2.5 kb BamHI-EcoRI fragment containing the CG52643-05 sequence was subcloned into BamHI-EcoRI digested pBlueBac4.5/V5-His (CuraGen Corporation) insect expression vector to generate plasmid 2599. Following standard procedures (Invitrogen pBlueBac protocol), recombinant baculovirus was generated and plaque-purified. Fresh Sf9 cells in adherent culture were infected with the recombinant baculovirus. The culture media were harvested 5 days post-infection and assayed for CG52643-05 protein expression by Western blot under reducing conditions using an anti-V5 antibody. CG52643-05 is expressed as 111 kDa protein.

### Example D3

### Expression of CGS1051-06 in Human Embryonic Kidney 293 Cells

A 1.29 kb BamHI-XhoI fragment containing the CG51051-06 sequence was subeloned into BamHI-XhoI digested pCEP4/Sec vector to generate plasmid 209. The resulting plasmid 209 was transfected into 293 cells using the LipofectaminePlus reagent following the manufacturer's instructions (Gibco/BRL). The cell pellet and supernatant were harvested 72 h post transfection and examined for CG51051-06 expression by Western blot (reducing conditions) using an anti-V5 antibody. CG51051-06 is expressed as a 60 kDa protein secreted by 293 cells.

### Example D4

### Epithelial Cell Survival Assay (PE51a1)

Netrins are a family of guidance molecules that act to both attract and repel the growing axons of a broad range of neuronal cell types during development and are also involved in controling neuronal cell migration. Netrin signaling occurs through specific receptor complexes containing either the colorectal cancer (DCC) or neogenin protein (attractive receptor), or the UNC-5-related proteins (repellent receptor). Netrin-DCC signaling has also been shown to regulate cell death in epithelial cells in vitro, raising the interesting possibility that netrins may also regulate cell death in the developing nervous system (Livesey et al., Cell. Mol. Life Sci. Oct. 1, 1999;56(1-2):62-8). CG51051-06 is related to the netrin family of neuronal guidance molecules related to neuronal spreading, migration, development and survival. CG51051-06 may act as a chemotrophic/survival potentiating factor in neuronal repair or regeneration.

BrdU Incorporation. Proliferative activity is measured by treatment of serum-starved cultured cells with a given agent and measurement of BRDU incorporation during DNA synthesis. 789-0 and 769-P kidney epithelial cells were cultured in DMEM supplemented with 10% fetal bovine serum or 10% calf serum respectively. Cells were grown to confluence at 37° C. in 10% CO₂/air. Cells were then starved in DMEM for 24-72 h. pCEP4sec or pCEP4sec/CG51051-06 enriched conditioned medium was added (10 μL/100 μL of culture) for 18 h. BrdU (10 μM final concentration) was then added and incubated with the cells for 5 h. BrdU incorporation was assayed according to the manufacturer's specifications (Boehringer Mannheim, Indianapolis, Ind.).

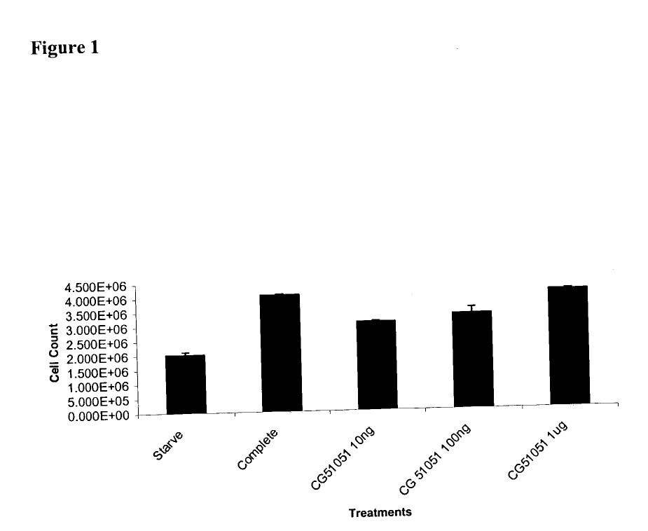

CG51051-06 has shown BrdU incorporation activity on 786-0 and 769-P kidney epithelial cells. As shown in FIG. 1, CG51051-06 promotes growth and survival of epithelial cells demonstrating that the molecule is functional and may act as a chemotrophic/survival potentiating factor in neuronal repair or regeneration.

### Example D5

### Expression of CG51051-07 in Human Embryonic Kidney 293 Cells

A 1.5 kb fragment containing the CG51051-07 sequence was subcloned into pCEP4-Sec-GATEWAY vector to generate plasmid 1729. The resulting plasmid 1729 was transfected into 293 cells using the LipofectaminePlus reagent following the manufacturer's instructions (Gibco/BRL). The cell pellet and supernatant were harvested 72 h post transfection and examined for CG51051-07 expression by Western blot (reducing conditions) using an anti-V5 antibody. CG51051-07 is expressed as a 67 kDa protein secreted by 293 cells.

### Example D6

### Expression of CG52643-05 in Stable CHO-K1 Cells

A 2548 bp long BamHI-EcoRI fragment containing the CG52643-05 sequence was subcloned into BamHI-EcoRI digested pEE14.4FL2_MSA to generate plasmid 2809. The resulting plasmid 2809 was transfected into CHO-K1 cells using the LipofectaminePlus reagent following the manufacturer's instructions (Invitrogen/Gibco Stable clones were selected based on resistance against methionine sulfoximine. The expression and secretion levels of the selected clones were assessed by Western blot analysis using HRP conjugated V5 antibody. (The V5 epitope is fused to the gene of interest at the Cter, in the pEE14.4Sec vector.) The CG52643-05 protein is secreted as a 178.4 kDa protein.

### Example D7

### Expression of CG52643-05 using Baculovirus Expression System.

A 2.5 kb BamHI-EcoRI fragment containing the CG52643-05 sequence was subcloned into BamHI-EcoRI digested pBlueBac4.5/V5-His (CuraGen Corporation) insect expression vector to generate plasmid 2599. Following standard procedures (Invitrogen pBlueBac protocol), recombinant baculovirus was generated and plaque-purified. Fresh Sf9 cells in adherent culture were infected with the recombinant baculovirus. The culture media were harvested 5 days post-infection and assayed for CG52643-05 protein expression by Western blot under reducing conditions using an anti-V5 antibody. CG52643-05 is expressed as 111 kDa protein.

### OTHER EMBODIMENTS

Although particular embodiments are disclosed herein in detail, this is done by way of example for purposes of illustration only, and is not intended to be limiting with respect to the scope of the appended claims, which follow. In particular, it is contemplated by the inventors that various substitutions, alterations, and modifications will be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims. The claims presented are representative of the inventions disclosed herein. Other, unclaimed inventions are also contemplated. Applicants reserve the right to pursue such inventions in later claims.

What is claimed is:

1. An isolated polypeptide comprising the mature form of an amino acid sequenced selected from the group consisting of SEQ ID NO:2n, wherein n is an integer between 1 and 174.

2. An isolated polypeptide comprising an amino acid sequence selected from the group consisting of SEQ ID NO:2n, wherein n is an integer between 1 and 174.

3. An isolated polypeptide comprising an amino acid sequence which is at least 95% identical to an amino acid sequence selected from the group consisting of SEQ ID NO:2n, wherein n is an integer between 1 and 174.

4. An isolated polypeptide, wherein the polypeptide comprises an amino acid sequence comprising one or more conservative substitutions in the amino acid sequence selected from the group consisting of SEQ ID NO:2n, wherein n is an integer between 1 and 174.

5. The polypeptide of claim 1 wherein said polypeptide is naturally occurring.

6. A composition comprising the polypeptide of claim 1 and a carrier.

7. A kit comprising, in one or more containers, the composition of claim 6.

8. The use of a therapeutic in the manufacture of a medicament for treating a syndrome associated with a human disease, the disease selected from a pathology associated with the polypeptide of claim 1, wherein the therapeutic comprises the polypeptide of claim 1.

9. A method for determining the presence or amount of the polypeptide of claim 1 in a sample, the method comprising:
(a) providing said sample;
(b) introducing said sample to an antibody that binds immunospecifically to the polypeptide; and
(c) determining the presence or amount of antibody bound to said polypeptide, thereby determining the presence or amount of polypeptide in said sample.

10. A method for determining the presence of or predisposition to a disease associated with altered levels of expression of the polypeptide of claim 1 in a first mammalian subject, the method comprising:
a) measuring the level of expression of the polypeptide in a sample from the first mammalian subject; and
b) comparing the expression of said polypeptide in the sample of step (a) to the expression of the polypeptide present in a control sample from a second mammalian subject known not to have, or not to be predisposed to, said disease,
wherein an alteration in the level of expression of the polypeptide in the first subject as compared to the control sample indicates the presence of or predisposition to said disease.

11. A method of identifying an agent that binds to the polypeptide of claim 1, the method comprising:
(a) introducing said polypeptide to said agent; and
(b) determining whether said agent binds to said polypeptide.

12. The method of claim 11 wherein the agent is a cellular receptor or a downstream effector.

13. A method for identifying a potential therapeutic agent for use in treatment of a pathology, wherein the pathology is related to aberrant expression or aberrant physiological interactions of the polypeptide of claim 1, the method comprising:
(a) providing a cell expressing the polypeptide of claim 1 and having a property or function ascribable to the polypeptide;
(b) contacting the cell with a composition comprising a candidate substance; and
(c) determining whether the substance alters the property or function ascribable to the polypeptide;
whereby, if an alteration observed in the presence of the substance is not observed when the cell is contacted with a composition in the absence of the substance, the substance is identified as a potential therapeutic agent.

14. A method for screening for a modulator of activity of or of latency or predisposition to a pathology associated with the polypeptide of claim 1, said method comprising:
(a) administering a test compound to a test animal at increased risk for a pathology associated with the polypeptide of claim 1, wherein said test animal recombinantly expresses the polypeptide of claim 1;
(b) measuring the activity of said polypeptide in said test animal after administering the compound of step (a); and
(c) comparing the activity of said polypeptide in said test animal with the activity of said polypeptide in a control animal not administered said polypeptide, wherein a change in the activity of said polypeptide in said test animal relative to said control animal indicates the test compound is a modulator activity of or latency or predisposition to, a pathology associated with the polypeptide of claim 1.

15. The method of claim 14, wherein said test animal is a recombinant test animal that expresses a test protein transgene or expresses said transgene under the control of a promoter at an increased level relative to a wild-type test animal, and wherein said promoter is not the native gene promoter of said transgene.

16. A method for modulating the activity of the polypeptide of claim 1, the method comprising contacting a cell sample expressing the polypeptide of claim 1 with a compound that binds to said polypeptide in an amount sufficient to modulate the activity of the polypeptide.

17. A method of treating or preventing a pathology associated with the polypeptide of claim 1, the method comprising administering the polypeptide of claim 1 to a subject in which such treatment or prevention is desired in an amount sufficient to treat or prevent the pathology in the subject.

18. The method of claim 17, wherein the subject is a human.

19. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising the amino acid sequence selected from the group consisting of SEQ ID NO:2n, wherein n is an integer between 1 and 174 or a biologically active fragment thereof.

20. An isolated nucleic acid molecule comprising a nucleic acid sequence selected from the group consisting of SEQ ID NO:2n−1, wherein n is an integer between 1 and 174.

21. The nucleic acid molecule of claim 20, wherein the nucleic acid molecule is naturally occurring.

22. A nucleic acid molecule, wherein the nucleic acid molecule differs by a single nucleotide from a nucleic acid sequence selected from the group consisting of SEQ ID NO: 2n−1, wherein n is an integer between 1 and 174.

23. An isolated nucleic acid molecule encoding the mature form of a polypeptide having an amino acid sequence selected from the group consisting of SEQ ID NO:2n, wherein n is an integer between 1 and 174.

24. An isolated nucleic acid molecule comprising a nucleic acid selected from the group consisting of 2n−1, wherein n is an integer between 1 and 174.

25. The nucleic acid molecule of claim 20, wherein said nucleic acid molecule hybridizes under stringent conditions to the nucleotide sequence selected from the group consisting of SEQ ID NO: 2n−1, wherein n is an integer between 1 and 174, or a complement of said nucleotide sequence.

26. A vector comprising the nucleic acid molecule of claim 20.

27. The vector of claim 26, further comprising a promoter operably linked to said nucleic acid molecule.

28. A cell comprising the vector of claim 26.

29. An antibody that immunospecifically binds to the polypeptide of claim 1.

30. The antibody of claim 29, wherein the antibody is a monoclonal antibody.

31. The antibody of claim 29, wherein the antibody is a humanized antibody.

32. A method for determining the presence or amount of the nucleic acid molecule of claim 20 in a sample, the method comprising:
(a) providing said sample;
(b) introducing said sample to a probe that binds to said nucleic acid molecule; and
(c) determining the presence or amount of said probe bound to said nucleic acid molecule,
thereby determining the presence or amount of the nucleic acid molecule in said sample.

33. The method of claim 32 wherein presence or amount of the nucleic acid molecule is used as a marker for cell or tissue type.

34. The method of claim 33 wherein the cell or tissue type is cancerous.

35. A method for determining the presence of or predisposition to a disease associated with altered levels of expression of the nucleic acid molecule of claim 20 in a first mammalian subject, the method comprising:
a) measuring the level of expression of the nucleic acid in a sample from the first mammalian subject; and
b) comparing the level of expression of said nucleic acid in the sample of step (a) to the level of expression of the nucleic acid present in a control sample from a second mammalian subject known not to have or not be predisposed to, the disease;
wherein an alteration in the level of expression of the nucleic acid in the first subject as compared to the control sample indicates the presence of or predisposition to the disease.

36. A method of producing the polypeptide of claim 1, the method comprising culturing a cell under conditions that lead to expression of the polypeptide, wherein said cell comprises a vector comprising an isolated nucleic acid molecule comprising a nucleic acid sequence selected from the group consisting of SEQ ID NO:2n−1, wherein n is an integer between 1 and 174.

37. The method of claim 36 wherein the cell is a bacterial cell.

38. The method of claim 36 wherein the cell is an insect cell.

39. The method of claim 36 wherein the cell is a yeast cell.

40. The method of claim 36 wherein the cell is a mammalian cell.

41. A method of producing the polypeptide of claim 2, the method comprising culturing a cell under conditions that lead to expression of the polypeptide, wherein said cell comprises a vector comprising an isolated nucleic acid molecule comprising a nucleic acid sequence selected from the group consisting of SEQ ID NO:2n−1, wherein n is an integer between 1 and 174.

42. The method of claim 41 wherein the cell is a bacterial cell.

43. The method of claim 41 wherein the cell is an insect cell.

44. The method of claim 41 wherein the cell is a yeast cell.

45. The method of claim 41 wherein the cell is a mammalian cell.
